(19) United States
(12) Patent Application Publication
(10) Pub. No.: US 2024/0116985 A1
(43) Pub. Date: Apr. 11, 2024

(54) STAPLED PEPTIDES AND METHODS THEREOF

(71) Applicant: FOG PHARMACEUTICALS, INC., Cambridge, MA (US)

(72) Inventors: Brian Halbert White, Malden, MA (US); Yaguang Si, Arlington, MA (US); Martin Robert Tremblay, Melrose, MA (US); Deborah Gail Conrady, Arlington, MA (US); Yue-Mei Zhang, Wellesley, MA (US); Ivan Tucker Jewett, Boston, MA (US); Lorenzo Josue Alfaro-Lopez, Belmont, MA (US); Sarah Isabelle Cappucci, Manchester, NH (US); Zhi Li, Braintree, MA (US); John Hanney McGee, Somerville, MA (US)

(21) Appl. No.: 18/017,024

(22) PCT Filed: Jul. 22, 2021
(86) PCT No.: PCT/US21/42856

§ 371 Date: Jan. 19, 2023

Related U.S. Application Data
(60) Provisional application No. 63/208,494, filed on Jun. 8, 2021.
(60) Provisional application No. 63/055,308, filed on Jul. 22, 2020.

(51) Int. Cl. C07K 7/08; A61K 45/06; A61P 35/00; A61K 38/00

(52) U.S. Cl. CPC C07K 7/08; A61K 45/06; A61P 35/00; A61K 38/00

(57) ABSTRACT

Among other things, the present disclosure provides various agents. In some embodiments, provided agents can bind to beta-catenin. In some embodiments, the present disclosure provides technologies for modulating beta-catenin functions. In some embodiments, the present disclosure provides technologies for preventing and/or treating conditions, disorders or diseases associated with beta-catenin. In some embodiments, the present disclosure provides designed amino acids which can provide improved properties and/or activities. In some embodiments, the present disclosure provides agents comprising such amino acids.

### CROSS-REFERENCE TO RELATED APPLICATIONS

This application is a National Stage Entry of International Application No. PCT/US21/42856, filed Jul. 22, 2021, which claims priority to United States Provisional Application Nos. 63/055,308, filed Jul. 22, 2020, and 63/208,494, filed Jun. 8, 2021, the entirety of each of which is incorporated herein by reference.

### SEQUENCE LISTING

The instant application contains a Sequence Listing which has been submitted electronically in ASCII format and is hereby incorporated by reference in its entirety. Said ASCII copy, created on Jul. 20, 2023, is named SL.txt and is 1,896,321 bytes in size.

### BACKGROUND

Stapled peptides are useful for various applications. For example, as biologically active agents, they can be utilized to modulate various biological functions.

### SUMMARY

Among other things, the present disclosure provides powerful technologies (e.g., agents (e.g., those that are or comprise peptides, in many embodiments, stapled peptides), compositions, methods, etc.) for modulating various biological functions. In some embodiments, provided technologies comprise designed structural features, e.g., novel amino acid residues, that can provide significantly improved properties and/or activities compared to comparable reference technologies that do not contain such designed structural features. In some embodiments, the present disclosure provides designed amino acids as described herein, whose incorporation into peptide agents, including stapled peptides, can provide significantly improved properties and/or activities such as improved lipophilicity and/or delivery into cells compared to reference amino acids (e.g., Asp). In some embodiments, the present disclosure provides technologies including peptides comprising such designed amino acid residues. In some embodiments, the present disclosure provides stapled peptides comprise such designed amino acid residues.

In some embodiments, the present disclosure provides technologies for modulating one or more functions of beta-catenin. Particularly, in some embodiments, the present disclosure provides various peptides, in many instances stapled peptides, that can bind to beta-catenin. Particularly, in some embodiments, the present disclosure provides various agents, e.g., peptides, in many instances stapled peptides, that can bind to beta-catenin and modulate its functions. As demonstrated herein, in some embodiments, the present disclosure binds agents that can interact with beta-catenin at a unique set of residues. In some embodiments, a binding site comprises one or more or all of the set of residues. In some embodiments, provided agents interact with one or more of a set of residues that are or correspond to the following residues of SEQ ID NO: 1: A305, Y306, G307, N308, Q309, K312, R342, K345, V346, V349, Q375, R376, Q379, N380, L382, W383, R386, N387, D413, N415, V416, T418, and C419. In some embodiments, provided agents interact with one or more of amino acid residue that are or correspond to A305, Y306, G307, N308, Q309, K312, R342, K345, V346, V349, Q375, Q379, N380, L382, W383, R386, N387, D413, N415, V416, T418, and C419 of SEQ ID NO: 1. In some embodiments, provided agents interact with one or more of amino acid residues that are or correspond to A305, Y306, G307, N308, Q309, K312, K345, V346, V349, Q379, N380, L382, W383, R386, N387, D413, N415, V416, T418, and C419 of SEQ ID NO: 1. In some embodiments, provided agents interact with one or more of amino acid residues that are or correspond to G307, K312, K345, W383, N387, D413, and N415 of SEQ ID NO: 1. In some embodiments, provided agents interact with one or more of amino acid residues that are or correspond to K312, K345, R386 and W383 of SEQ ID NO: 1. In some embodiments, provided agents interact with one or more of amino acid residues that are or correspond to K312, K345 and W383 of SEQ ID NO: 1.

As demonstrated herein, provided technologies can modulate one or more biological processes associated with beta-catenin. In some embodiments, provided agents compete with a ligand for a particular binding site (e.g., with a member of the T cell factor/lymphoid enhancer factor (TCF/LEF) family of transcription factors at the TCF site on beta-catenin). In some embodiments, provided technologies compete with TCF for interactions with beta-catenin. In some embodiments, binding of provided agents to a beta-catenin site decreases, suppresses and/or blocks binding to beta-catenin by another binding partner (e.g., a kinase). In some embodiments, binding of provided agents, e.g., at a TCF site, blocks binding of beta-catenin by a TCF/LEF family member. In some embodiments, the present disclosure provides agents that can bind to a site of beta-catenin, e.g., a TCF binding site of beta-catenin, selectively over one of more other potential binding sites of beta-catenin (e.g., for other ligands such as peptides, proteins, etc.; in some embodiments, a ligand is Axin; in some embodiments, a ligand is Bc19). In some embodiments, provided technologies modulate one or more beta-catenin functions associated with its interactions with TCF. In some embodiments, provided technologies selectively modulate beta-catenin functions, e.g., functions associated with TCF interactions. In some embodiments, provided technologies selectively modulate beta-catenin functions and do not significantly impact functions that are not associated with beta-catenin (e.g., various functions and/or processes in the Wnt pathway that are not associated with beta-catenin). In some embodiments, provided technologies are useful for inhibiting beta-catenin functions. In some embodiments, provided technologies are usefully for promoting and/or enhancing immune activities, e.g., anti-tumor adaptive immunity.

In some embodiments, provided technologies are useful for preventing or treating various conditions, disorders or diseases including cancer. In some embodiments, the present disclosure provides methods for treating or preventing a condition, disorder or disease associated with beta-catenin, comprising administering to a subject suffered therefrom or susceptible thereto an effective amount of a provided agent or a pharmaceutically acceptable salt thereof. In some embodiments, a condition, disorder or disease is associated with beta-catenin's interactions with TCF. In some embodiments, an agent, e.g., a staple peptide, is administered as a pharmaceutical composition. In some embodiments, the present disclosure provides pharmaceutical compositions which comprise or deliver a provided agent or a pharmaceutically acceptable salt thereof. In some embodiments, a pharmaceutical composition further comprises a lipid. As demonstrated herein, in some embodiments, a suitable lipid can promote delivery/activities. In some embodiments, an agent is or comprises a peptide. In some embodiments, an agent is or comprises a stapled peptides. In some embodiments, provided agents that can bind beta-catenin comprise one or more designed amino acid residues.

In some embodiments, the present disclosure provides agents that bind to a polypeptide comprising or consisting of SEQ ID NO: 1 (Uniprot ID P35222), or residues 250-450 of SEQ ID NO: 1, or residues 305-419 of SEQ ID NO: 1:

Uniprot No. P35222
(SEQ ID NO: 1)
MATQADLMELDMAMEPDRKAAVSHWQQQSYLDSGIHSGATTTAPSLSGKG
NPEEEDVDTSQVLYEWEQGFSQSFTQEQVADIDGQYAMTRAQRVRAAMFP
ETLDEGMQIPSTQFDAAHPTNVQRLAEPSQMLKHAVVNLINYQDDAELAT
RAIPELTKLLNDEDQVVVNKAAVMVHQLSKKEASRHAIMRSPQMVSAIVR
TMQNTNDVETARCTAGTLHNLSHHREGLLAIFKSGGIPALVKMLGSPVDS
VLFYAITTLHNLLLHQEGAKMAVRLAGGLQKMVALLNKTNVKFLAITTDC
LQILAYGNQESKLIILASGGPQALVNIMRTYTYEKLLWTTSRVLKVLSVC
SSNKPAIVEAGGMQALGLHLTDPSQRLVQNCLWTLRNLSDAATKQEGMEG
LLGTLVQLLGSDDINVVTCAAGILSNLTCNNYKNKMMVCQVGGIEALVRT
VLRAGDREDITEPAICALRHLTSRHQEAEMAQNAVRLHYGLPVVVKLLHP
PSHWPLIKATVGLIRNLALCPANHAPLREQGAIPRLVQLLVRAHQDTQRR
TSMGGTQQQFVEGVRMEEIVEGCTGALHILARDVHNRIVIRGINTIPLFV
QLLYSPIENIQRVAAGVLCELAQDKEAAEAIEAEGATAPLTELLHSRNEG
VATYAAAVLERMSEDKPQDYKKRLSVELTSSLERTEPMAWNETADLGLDI
GAQGEPLGYRQDDPSYRSFHSGGYGQDALGMDPMMEHEMGGHHPGADYPV
DGLPDLGHAQDLMDGLPPGDSNQLAWEDTDL

In some embodiments, provided agents specifically interact with one or more residues which are or correspond to residues 305-419 of SEQ ID NO: 1. In some embodiments, provided agents specifically bind to a motif (e.g., a portion of a polypeptide, a domain of a polypeptide, etc.) that comprise one or more residues corresponding to Ala305, Tyr306, Gly307, Asn 308, Gln309, Lys312, Arg342, Lys345, Val346, Val349, Gln375, Arg376, Gln379, Asn380, Leu382, Trp383, Arg386, Asn387, Asp413, Asn415, Val416, Thr418, and Cys419 of SEQ ID NO: 1. In some embodiments, provided agents specifically bind to a motif (e.g., a portion of a polypeptide, a domain of a polypeptide, etc.) that comprise one or more residues corresponding to Ala305, Tyr306, Gly307, Asn 308, Gln309, Lys312, Lys345, Val346, Val349, Gln375, Arg376, Gln379, Asn380, Leu382, Trp383, Arg386, Asn387, Asp413, Asn415, Val416, Thr418, and Cys419 of SEQ ID NO: 1. In some embodiments, an agent binds to a motif comprising one or more of the following residues within SEQ ID NO: 1: Ala305, Tyr306, Gly307, Asn 308, Gln309, Lys312, Arg342, Lys345, Val346, Val349, Gln375, Arg376, Gln379, Asn380, Leu382, Trp383, Arg386, Asn387, Asp413, Asn415, Val416, Thr418, and Cys419. In some embodiments, an agent specifically binds to a motif comprising one or more of the following residues within SEQ ID NO: 1: Ala305, Tyr306, Gly307, Asn 308, Gln309, Lys312, Lys345, Val346, Val349, Gln375, Arg376, Gln379, Asn380, Leu382, Trp383, Arg386, Asn387, Asp413, Asn415, Val416, Thr418, and Cys419. In some embodiments, an agent binds to a motif comprising one or more of the following residues within SEQ ID NO: 1: Ala305, Tyr306, Gly307, Asn 308, Gln309, Lys312, Arg342, Lys345, Val346, Val349, Gln 375, Gln379, Asn380, Leu382, Trp383, Arg386, Asn387, Asp413, Asn415, Val416, Thr418, and Cys419. In some embodiments, an agent binds to a motif comprising one or more of the following residues within SEQ ID NO: 1: Ala305, Tyr306, Gly307, Asn 308, Gln309, Lys312, Lys345, Val346, Val349, Gln379, Asn380, Leu382, Trp383, Arg386, Asn387, Asp413, Asn415, Val416, Thr418, and Cys419. In some embodiments, provided technologies bind to a motif comprising at least 2, 3, 4, 5, 6, or 7 of G307, K312, K345, W383, N387, D413, and N415. In some embodiments, provided agents specifically bind to such motifs. In some embodiments, a motif may be referred to as a binding site. In some embodiments, provided technologies selectively bind to such a binding site over an Axin binding site. In some embodiments, provided technologies selectively bind to such a binding site over a Bcl9 binding site. In some embodiments, provided technologies selectively bind to such a binding site over a TCF binding site. In some embodiments, provided technology binds to such a binding site in a reverse N to C direction compared to TCF. In some embodiments, provided technologies do not bind to Axin binding site of beta-catenin. In some embodiments, provided technologies do not bind to Bcl9 binding site of beta-catenin. Various technologies, e.g., crystallography, NMR, biochemical assays, etc., may be utilized to assess interactions with beta-catenin in accordance with the present disclosure.

In some embodiments, there are two amino acid residues between two amino acid residues bonded to the same staple. Such a staple may be referred to as a (i, i+3) staple. Similarly, in some embodiments, there are 3, 4, 5, 6, 7, 8, 9, or 10 amino acid residues between two amino acid residues bonded to the same staple, and such a staple may be referred to as a (i, i+4), (i, i+5), (i, i+6), (i, i+7), (i, i+8), (i, i+9), (i, i+10), or (i, i+11) staple, respectively. In some embodiments, a staple is (i, i+3). In some embodiments, a staple is (i, i+4). In some embodiments, a staple is (i, i+7). In some embodiments, there are two staples in a provided agent. In some embodiments, one staple is (i, i+3) and the other is (i, i+7).

In some embodiments, the present disclosure provides an agent of formula I:

R^{N}-L^{P1}-L^{AA1}-L^{P2}L^{AA2}-L^{P3}-L^{AA3}-L^{P4}-L^{AA4}-L^{P5}-L^{AA5}-L^{P6}-L^{AA6}-L^{P7}-R^{C} I

or a salt thereof, wherein each variable is independently as described herein.

In some embodiments, the present disclosure provides an agent which is or comprises:

X¹X²X³X⁴X⁵X⁶X⁷X⁸X⁹X¹⁰X¹¹X¹²X¹³,

wherein:
each of X¹, X², X³, X⁴, X⁵, X⁶, X⁷, X⁸, X⁹, X¹⁰, X¹¹, X¹², and X¹³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group; and
X¹³ comprises a side chain comprising an optionally substituted aromatic group.

In some embodiments, the present disclosure provides an agent which is or comprises:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X¹² comprises a side chain comprising an optionally substituted aromatic group; and
X¹³ comprises a side chain comprising an optionally substituted aromatic group.

In some embodiments, the present disclosure provides an agent which is or comprises:

[X]pX¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X]p′;

wherein:
each of p14, p15, p¹⁶ and p17 is independently 0 or 1;
each of p and p′ is independently 0-10;
each of X, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X¹² comprises a side chain comprising an optionally substituted aromatic group; and
X¹³ comprises a side chain comprising an optionally substituted aromatic group.

In some embodiments, an agent is [X]pX¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X]p′. In some embodiments, an agent is R^{N}—[X]pX¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X]p′-R^{C}.

In some embodiments, the present disclosure provides an agent which is or comprises:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X¹² comprises a side chain comprising an optionally substituted aromatic group; and
X¹³ comprises a side chain comprising an optionally substituted aromatic group.

In some embodiments, an agent is or comprises a peptide. In some embodiments, an agent is or comprises a stapled peptide. In some embodiments, X¹ and X⁴, and/or X⁴ and X¹¹ are independently amino acid residues suitable for stapling, or are stapled, X³ and X¹⁰ independently amino acid residues suitable for stapling, or are stapled, X¹ and X⁴, and/or X¹⁰ and X¹⁴ are independently amino acid residues suitable for stapling, or are stapled, or X¹ and X⁴, and/or X⁷ and X¹⁴ are independently amino acid residues suitable for stapling, or are stapled. In some embodiments, X¹ and X⁴ are independently amino acid residues suitable for stapling. In some embodiments, X¹ and X⁴ are stapled. In some embodiments, X⁴ and X¹¹ are independently amino acid residues suitable for stapling. In some embodiments, X⁴ and X¹¹ are stapled. In some embodiments, X¹⁰ and X¹⁴ are independently amino acid residues suitable for stapling. In some embodiments, X¹⁰ and X¹⁴ are stapled. In some embodiments, X⁷ and X¹⁴ are independently amino acid residues suitable for stapling. In some embodiments, X⁷ and X¹⁴ are stapled. In some embodiments, X¹ and X⁴, and X¹⁰ and X¹⁴ are independently amino acid residues suitable for stapling. In some embodiments, X¹ and X⁴ are stapled, and X¹⁰ and X¹⁴ are stapled. In some embodiments, X¹ and X⁴, and X⁷ and X¹⁴ are independently amino acid residues suitable for stapling. In some embodiments, X¹ and X⁴ are stapled, and X⁷ and X¹⁴ are stapled. In some embodiments, X¹ and X⁴, and X⁴ and X¹¹ are independently amino acid residues suitable for stapling. In some embodiments, a stapled peptide is a stitched peptide comprising two or more staples, some of which may bond to the same backbone atom. In some embodiments, X¹ and X⁴ are stapled, and X⁴ and X¹¹ are stapled. In some embodiments, a staple connecting X¹ and X⁴ and a staple connecting X⁴ and X¹¹ are bonded to a common backbone atom of X⁴. In some embodiments, a common backbone atom is the alpha-carbon of X⁴. In some embodiments, X³ and X¹⁰ are independently amino acid residues suitable for stapling. In some embodiments, X³ and X¹⁰ are stapled.

In some embodiments, the present disclosure provides agents that bind to a polypeptide comprising or consisting of residues 305-419 of SEQ ID NO: 1. In some embodiments, an agent has a molecular mass of no more than about 5000 Daltons. In some embodiments, it is no more than about 2500, 3000, 3500, 4000, 4500 or 5000 Daltons. In some embodiments, it is no more than about 2500 Daltons. In some embodiments, it is no more than about 3000 Daltons. In some embodiments, it is no more than about 3500 Daltons. In some embodiments, it is no more than about 4000 Daltons. In some embodiments, it is no more than about 500 Daltons.

In some embodiments, the present disclosure provides various reagents and methods associated with provided agents including, for example, reagents and/or systems for identifying, characterizing and/or assessing them, strategies for preparing them, and various diagnostic and therapeutic methods relating to them.

In some embodiments, the present disclosure provides pharmaceutical compositions comprising or delivering a provided agent and a pharmaceutical acceptable carrier. In some embodiments, a provided agent is a pharmaceutically acceptable salt form. In some embodiments, a provided composition comprises a pharmaceutically acceptable salt form an agent. In some embodiments, in various compositions and methods, agents are provided as pharmaceutically acceptable salt forms.

In some embodiments, the present disclosure provides methods for modulating a property, activity and/or function of beta-catenin, comprising contacting beta-catenin with a provided agent. In some embodiments, the present disclosure provides methods for modulating a property, activity and/or function of beta-catenin in a system comprising beta-catenin, comprising administering to a system an effective amount of a provided agent. In some embodiments, the present disclosure provides methods for modulating a property, activity and/or function of beta-catenin in a system expressing beta-catenin, comprising administering or delivering to a system an effective amount of a provided agent. In some embodiments, an activity of beta-catenin is inhibited or reduced. In some embodiments, a function of beta-catenin is inhibited or reduced. In some embodiments, a property, activity and/or function is associated with beta-catenin/TCF interaction.

In some embodiments, the present disclosure provides methods for modulating beta-catenin/TCF interaction. In some embodiments, the present disclosure provides methods for modulating beta-catenin/TCF interaction, comprising contacting beta-catenin with a provided agent. In some embodiments, the present disclosure provides methods for modulating beta-catenin/TCF interaction in a system comprising beta-catenin and TCF, comprising administering or delivering to the system an effective amount a provided agent. In some embodiments, the present disclosure provides methods for modulating beta-catenin/TCF interaction in a system expressing beta-catenin and TCF, comprising administering or delivering to the system an effective amount a provided agent. In some embodiments, interactions between beta-catenin and TCF is reduced. In some embodiments, interactions between beta-catenin and TCF is inhibited.

In some embodiments, the present disclosure provides methods for inhibiting cell proliferation, comprising administering or delivering to a population of cells an effective amount of a provided agent. In some embodiments, the present disclosure provides methods for inhibiting cell proliferation in a system, comprising administering or delivering to the system an effective amount of a provided agent. In some embodiments, the present disclosure provides methods for inhibiting cell growth, comprising administering or delivering to a population of cells an effective amount of a provided agent. In some embodiments, the present disclosure provides methods for inhibiting cell growth in a system, comprising administering or delivering to the system an effective amount of a provided agent. In some embodiments, such cell proliferation is beta-catenin dependent. In some embodiments, such cell growth is beta-catenin dependent. In some embodiments, such proliferation or growth is dependent on beta-catenin interactions with TCF.

In some embodiments, the present disclosure provides methods for reducing or preventing activation of a WNT pathway. In some embodiments, the present disclosure provides methods for reducing or preventing activation of a WNT pathway in a system, comprising administering or delivering to the system an effective amount of a provided agent.

In some embodiments, a system is in vitro. In some embodiments, a system is ex vivo. In some embodiments, a system is in vivo. In some embodiments, a system is or comprise a cell. In some embodiments, a system is or comprises a tissue. In some embodiments, a system is or comprises an organ. In some embodiments, a system is or comprises an organism. In some embodiments, a system is an animal. In some embodiments, a system is human. In some embodiments, a system is or comprises cells, tissues or organs associated with a condition, disorder or disease. In some embodiments, a system is or comprises cancer cells.

In some embodiments, the present disclosure provides methods for preventing conditions, disorders or diseases. In some embodiments, the present disclosure provides methods for reducing risks of conditions, disorders or diseases. In some embodiments, the present disclosure provides methods for preventing a condition, disorder or disease, comprising administering or delivering to a subject susceptible thereto an effective amount of an agent of the present disclosure. In some embodiments, the present disclosure provides methods for reducing risk of a condition, disorder or disease, comprising administering or delivering to a subject susceptible thereto an effective amount of an agent of the present disclosure. In some embodiments, the present disclosure provides methods for reducing risks of a condition, disorder or disease in a population, comprising administering or delivering to a population of subjects susceptible thereto an effective amount of an agent of the present disclosure. In some embodiments, the present disclosure provides methods for treating conditions, disorders or diseases. In some embodiments, the present disclosure provides methods for treating a condition, disorder or disease, comprising administering or delivering to a subject suffering therefrom an effective amount of an agent of the present disclosure. In some embodiments, a symptom is reduced, removed or prevented. In some embodiments, one or more parameters for assessing a condition, disorder or disease are improved. In some embodiments, survival of subjects are extended. As appreciated by those skilled in the art, in some embodiments, prevention, reduced risks, and/or effects of treatment may be assessed through clinical trials and may be observed in subject populations. In some embodiments, a condition, disorder or disease is cancer. In some embodiments, a condition, disorder or disease is associated with beta-catenin. In some embodiments, a condition, disorder or disease is associated with beta-catenin interaction with TCF. In some embodiments, a condition, disorder or disease is bladder cancer. In some embodiments, a condition, disorder or disease is endometrial cancer. In some embodiments, a condition, disorder or disease is adrenocortical carcinoma. In some embodiments, a condition, disorder or disease is gastric cancer. In some embodiments, a condition, disorder or disease is lung cancer. In some embodiments, a condition, disorder or disease is melanoma. In some embodiments, a condition, disorder or disease is esophageal cancer. In some embodiments, a condition, disorder or disease is colorectal cancer. In some embodiments, a cancer is liver cancer. In some embodiments, a cancer is prostate cancer. In some embodiments, a cancer is breast cancer. In some embodiments, a cancer is endometrial cancer.

In some embodiments, agents are administered as pharmaceutically compositions that comprise or deliver such agents. In some embodiments, agents are provided and/or delivered in pharmaceutically acceptable salt forms. In some embodiments, in a composition (e.g., a liquid composition of certain pH) an agent may exist in various forms including various pharmaceutically acceptable salt forms.

In some embodiments, a provided agent is utilized in combination with a second therapy. In some embodiments, a provided agent is utilized in combination with a second therapeutic agent. In some embodiments, a second therapy or therapeutic agent is administered prior to an administration or delivery of a provided agent. In some embodiments, a second therapy or therapeutic agent is administered at about the same time as an administration or delivery of a provided agent. In some embodiments, a second therapy or therapeutic agent is administered subsequently to an administration or delivery of a provided agent. In some embodiments, a subject is exposed to both a provided agent and a second therapeutic agent. In some embodiments, a subject is exposed to a therapeutic effect of a provided agent and a therapeutic effect of a second therapeutic agent. In some embodiments, a second therapy is or comprises surgery. In some embodiments, a second therapy is or comprises radiation therapy. In some embodiments, a second therapy is or comprises immunotherapy. In some embodiments, a second therapeutic agent is or comprises a drug. In some embodiments, a second therapeutic agent is or comprises a cancer drug. In some embodiments, a second therapeutic agent is or comprises a chemotherapeutic agent. In some embodiments, a second therapeutic agent is or comprises a hormone therapy agent. In some embodiments, a second therapeutic agent is or comprises a kinase inhibitor. In some embodiments, a second therapeutic agent is or comprises a checkpoint inhibitor (e.g., antibodies against PD1-, PD-L1, CTLA-4, etc.). In some embodiments, a provide agent can be administered with lower unit dose and/or total dose compared to being used alone. In some embodiments, a second agent can be administered with lower unit dose and/or total dose compared to being used alone. In some embodiments, one or more side effects associated with administration of a provided agent and/or a second therapy or therapeutic agent are reduced. In some embodiments, a combination therapy provides improved results, e.g., when compared to each agent utilized individually. In some embodiments, a combination therapy achieves one or more better results, e.g., when compared to each agent utilized individually.

Further description of certain embodiments of these aspects, and others, of the present disclosure, is presented below.

### BRIEF DESCRIPTION OF THE DRAWING

FIG. 1. Provided agents can modulate gene expression in cells. As demonstrated herein, provided peptides can effectively reduce expression of various nucleic acids such as Axin2 and Myc. For each gene, from left to right: I-796, I-849, and I-922.

FIG. 2. Provided technologies do not significantly impact expression of various beta-catenin-independent genes including various beta-catenin-independent WNT target genes such as PLOD2 and LCOR. For each gene, from left to right: I-796, I-849, and I-922.

FIG. 3. Provided technologies can effectively modulate expression. As shown in a report assay (I-849: square; I-922: triangle), provided peptides can effectively reduce expression compared to a control peptide I-796 (circle).

### DETAILED DESCRIPTION OF CERTAIN EMBODIMENTS

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity: As is known in the art, “affinity” is a measure of the tightness with a particular ligand (e.g., an agent) binds to its partner (e.g., beta-catenin or a portion thereof). Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety. In some embodiments, an agent is a compound. In some embodiments, an agent is a stapled peptide.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1−6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkylene: The term “alkylene” refers to a bivalent alkyl group.

Amino acid: In its broadest sense, as used herein, refers to any compound and/or substance that can be incorporated into a polypeptide chain, e.g., through formation of one or more peptide bonds. In some embodiments, an amino acid comprising an amino group and an a carboxylic acid group. In some embodiments, an amino acid has the structure of NH(R^{a1})-L^{a1}-C(R^{a2})(R^{a3})-L^{a2}-COOH, wherein each variable is independently as described in the present disclosure. In some embodiments, an amino acid has the general structure NH(R′)—C(R′)₂—COOH, wherein each R′ is independently as described in the present disclosure. In some embodiments, an amino acid has the general structure H₂N—C(R′)₂—COOH, wherein R′ is as described in the present disclosure. In some embodiments, an amino acid has the general structure H₂N—C(H)(R′)—COOH, wherein R′ is as described in the present disclosure. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, one or more hydrogens, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Animal: As used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Aryl: The term “aryl” used alone or as part of a larger moiety as in “aralkyl,” “aralkoxy,” “aryloxyalkyl,” etc. refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. In some embodiments, also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like, where a radical or point of attachment is on an aryl ring.

Associated with: Two events or entities are “associated” with one another, as that term is used herein, if the presence, level and/or form of one is correlated with that of the other. For example, a particular entity (e.g., nucleic acid (e.g., genomic DNA, transcripts, mRNA, etc.), polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population).

Binding: It will be understood that the term “binding”, as used herein, typically refers to a non-covalent association between or among agents. In many embodiments herein, binding is addressed with respect to particular agents and beta-catenin. It will be appreciated by those of ordinary skill in the art that such binding may be assessed in any of a variety of contexts. In some embodiments, binding is assessed with respect to beta-catenin. In some embodiments, binding is assessed with respect to one or more amino acid residues of beta-catenin. In some embodiments, binding is assessed with respect to one or more amino acid residues corresponding to (e.g., similarly positioned in three dimensional space and/or having certain similar properties and/or functions) those of beta-catenin.

Binding site: The term “binding site”, as used herein, refers to a region of a target polypeptide, formed in three-dimensional space, that includes one or more or all interaction residues of the target polypeptide. In some embodiments, “binding site” may refer to one or more amino acid residues which comprise or are one or more or all interaction amino acid residues of a target polypeptide. As will be understood by those of ordinary skill in the art, a binding site may include residues that are adjacent to one another on a linear chain, and/or that are distal to one another on a linear chain but near to one another in three-dimensional space when a target polypeptide is folded. A binding site may comprise amino acid residues and/or saccharide residues.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Cycloaliphatic: The term “cycloaliphatic,” as used herein, refers to saturated or partially unsaturated aliphatic monocyclic, bicyclic, or polycyclic ring systems having, e.g., from 3 to 30, members, wherein the aliphatic ring system is optionally substituted. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3−6 carbons. The terms “cycloaliphatic” may also include aliphatic rings that are fused to one or more aromatic or nonaromatic rings, such as decahydronaphthyl or tetrahydronaphthyl, where a radical or point of attachment is on an aliphatic ring. In some embodiments, a carbocyclic group is bicyclic. In some embodiments, a carbocyclic group is tricyclic. In some embodiments, a carbocyclic group is polycyclic. In some embodiments, “cycloaliphatic” (or “carbocycle” or “cycloalkyl”) refers to a monocyclic C₃-C₆ hydrocarbon, or a C₈-C₁₀ bicyclic hydrocarbon that is completely saturated or that contains one or more units of unsaturation, but which is not aromatic, or a C₉-C₁₆ tricyclic hydrocarbon that is completely saturated or that contains one or more units of unsaturation, but which is not aromatic.

Derivative: As used herein, the term “derivative” refers to a structural analogue of a reference substance. That is, a “derivative” is a substance that shows significant structural similarity with the reference substance, for example sharing a core or consensus structure, but also differs in certain discrete ways. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Dosageform or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is atherapeutic dosing regimen).

Engineered: In general, the term “engineered” refers to the aspect of having been manipulated by the hand of man. For example, in some embodiments, a peptide may be considered to be engineered if its amino acid sequence has been selected by man. For example, an engineered agent has an amino acid sequence that was selected based on preferences for corresponding amino acids at particular sites of protein-protein interactions. In some embodiments, an engineered sequence has an amino acid sequence that differs from the amino acid sequence of polypeptides included in the NCBI database that binds to a TCF site of beta-catenin. In many embodiments, provided agents are engineered agents. In some embodiments, engineered agents are peptide agents comprising non-natural amino acid residues, non-natural amino acid sequences, and/or peptide staples. In some embodiments, provided agents comprise or are engineered peptide agents which comprise engineered sequences.

Halogen: The term “halogen” means F, Cl, Br, or I.

Heteroaliphatic: The term “heteroaliphatic” is given its ordinary meaning in the art and refers to aliphatic groups as described herein in which one or more carbon atoms are replaced with one or more heteroatoms (e.g., oxygen, nitrogen, sulfur, silicon, phosphorus, and the like).

Heteroalkyl: The term “heteroalkyl” is given its ordinary meaning in the art and refers to alkyl groups as described herein in which one or more carbon atoms is replaced with a heteroatom (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-,” used alone or as part of a larger moiety, e.g., “heteroaralkyl,” or “heteroaralkoxy,” refer to monocyclic, bicyclic or polycyclic ring systems having, for example, a total of five to thirty, e.g., 5, 6, 9, 10, 14, etc., ring members, wherein at least one ring in the system is aromatic and at least one aromatic ring atom is a heteroatom. In some embodiments, a heteroatom is nitrogen, oxygen or sulfur. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where a radical or point of attachment is on a heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom” means an atom that is not carbon and is not hydrogen. In some embodiments, a heteroatom is oxygen, sulfur, nitrogen, phosphorus, boron or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR+(as in N-substituted pyrrolidinyl); etc.). In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur.

Heterocyclyl: As used herein, the terms “heterocycle,” “heterocyclyl,” “heterocyclic radical,” and “heterocyclic ring” are used interchangeably and refer to a monocyclic, bicyclic or polycyclic ring moiety (e.g., 3-30 membered) that is saturated or partially unsaturated and has one or more heteroatom ring atoms. In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where a radical or point of attachment is on a heteroaliphatic ring. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution ofone amino acid for another ofthe same type may often be considered a “homologous” substitution. Typical amino acid categorizations are summarized below (hydrophobicity scale of Kyte and Doolittle, 1982: A simple method for displaying the hydropathic character of a protein. Mol. Biol. 157:105-132):

| | | | | Side Chain | Hydropathy |
|---|---|---|---|---|---|
| | 3 Letter | 1 Letter | Side Chain | Acidity/ | Index of Kyte |
| Amino Acid | Code | Code | Polarity | Basicity | and Doolittle |
| Alanine | Ala | A | nonpolar | neutral | 1.8 |
| Arginine | Arg | R | polar | basic | −4.5 |
| Asparagine | Asn | N | polar | neutral | −3.5 |
| Aspartic acid | Asp | D | polar | acidic | −3.5 |
| Cysteine | Cys | C | nonpolar | neutral | 2.5 |
| Glutamic acid | Glu | E | polar | acidic | −3.5 |
| Glutamine | Gln | Q | polar | neutral | −3.5 |
| Glycine | Gly | G | nonpolar | neutral | −0.4 |
| Histidine | His | H | polar | basic | −3.2 |
| Isoleucine | Ile | I | nonpolar | neutral | 4.5 |
| Leucine | Leu | L | nonpolar | neutral | 3.8 |
| Lysine | Lys | K | polar | basic | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna.CMP matrix.

Interaction residues: The term “interaction residues”, “interaction motifs”, as used herein, refers to, with respect to an agent, residues or motifs in an agent that are designed to interact with particular target residues in a target polypeptide, or with respect to a target polypeptide, residues in a target polypeptide that interact with particular motifs (e.g., aromatic groups, amino acid residues, etc.) of an agent. Specifically, interaction residues and motifs of various agents are selected and arranged within the agents so that they will be displayed in three dimensional space within a predetermined distance (or volume) of identified target residues (e.g., upon binding, docking or other interaction assays). In many embodiments, interaction residues are direct-binding residues.

“Improved,” “increased” or “reduced”: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Partially unsaturated: As used herein, the term “partially unsaturated” refers to a moiety that includes at least one double or triple bond. The term “partially unsaturated” is intended to encompass groups having multiple sites of unsaturation, but is not intended to include aryl or heteroaryl moieties.

Peptide: The term “peptide” as used herein refers to a polypeptide. In some embodiments, a peptide is a polypeptide that is relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; RingeR's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other known methods such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, pharmaceutically acceptable salts include, but are not limited to, nontoxic base addition salts, such as those formed by acidic groups of provided compounds with bases. Representative alkali or alkaline earth metal salts include salts of sodium, lithium, potassium, calcium, magnesium, and the like. In some embodiments, pharmaceutically acceptable salts are ammonium salts (e.g., —N(R)₃+). In some embodiments, pharmaceutically acceptable salts are sodium salts. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. June 2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In some embodiments, suitable mono-protected amines include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. In some embodiments, suitable di-protected amines include amines that are substituted with two substituents independently selected from those described above as mono-protected amines, and further include cyclic imides, such as phthalimide, maleimide, succinimide, and the like. In some embodiments, suitable di-protected amines include pyrroles and the like, 2,2,5,5-tetramethyl-[1,2,5]azadisilolidine and the like, and azide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl. In some embodiments, suitable protected carboxylic acids include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, a-methoxybenzylidene ortho ester, 1-(N,N-dimethylamino)ethylidene derivative, a-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specificity: As is known in the art, “specificity” is a measure of the ability of a particular ligand (e.g., an agent) to distinguish its binding partner (e.g., beta-catenin) from other potential binding partners (e.g., another protein, another portion (e.g., domain) of beta-catenin.

Substitution: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. In some embodiments, example substituents are described below.

Suitable monovalent substituents are halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)N(R^{o})₂; —N(R^{o})C(S)N(R^{o})₂; —(CH₂)₀₋₄N(R^{o})C(O)OR^{o}; —N(R^{o})N(R^{o})C(O)R^{o}; —N(R^{o})N(R^{o})C(O)N(R^{o})₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSi(R^{o})₃; —(CH₂)₀₋₄OC(O)R^{o}; —OC(O)(CH₂)₀₋₄SR^{o}, —SC(S)SR^{o}; —(CH₂)₀₋₄SC(O)R^{o}; —(CH₂)₀₋₄C(O)N(R^{o})₂; —C(S)N(R^{o})₂; —C(S)SR^{o}; —SC(S)SR^{o}, —(CH₂)₀₋₄OC(O)N(R^{o})₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂N(R^{o})₂; —(CH₂)₀₋₄S(O)R^{o}; —N(R^{o})S(O)₂N(R^{o})₂; —N(R^{o})S(O)₂R^{o}; —N(OR^{o})R^{o}; —C(NH)N(R^{o})₂; —Si(R^{o})₃; —OSi(R^{o})₃; —P(R^{o})₂; —P(OR^{o})₂; —OP(R^{o})₂; —OP(OR^{o})₂; —N(R^{o})P(R^{o})₂; —B(R^{o})₂; —OB(R^{o})₂; —P(O)(R^{o})₂; —OP(O)(R^{o})₂; —N(R^{o})P(O)(R^{o})₂; —(C₁₋₄ straight or branched alkylene)O—N(R^{o})₂; or —(C₁₋₄ straight or branched alkylene)C(O)O—N(R^{o})₂; wherein each R^{o} may be substituted as defined below and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{o}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{o} (or the ring formed by taking two independent occurrences of R^{o} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5−6-membered saturated, partially unsaturated, or aryl ring having 0-4 heteroatoms independently selected from nitrogen, oxygen, and sulfur. Suitable divalent substituents on a saturated carbon atom of R^{o} include ═O and ═S.

Suitable divalent substituents are the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5−6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*2)₂₋₃O—, wherein each independent occurrence of R* is selected from hydrogen, C₁₋₆ aliphatic which may be substituted as defined below, or an unsubstituted 5−6-membered saturated, partially unsaturated, or aryl ring having 0-4 heteroatoms independently selected from nitrogen, oxygen, and sulfur.

Suitable substituents on the aliphatic group of R* are halogen, —R^{●}, -(haloR^{●}), —OH, —OR*^{●}, —O(haloR^{●}), —CN, —C(O)OH, —C(O)OR^{●}, —NH₂, —NHR^{●}, —NR^{●}₂, or —NO₂, wherein each R^{●}is unsubstituted or where preceded by “halo” is substituted only with one or more halogens, and is independently C₁₋₄ aliphatic, —CH₂Ph, —O(CH₂)₀₋₁Ph, or a 5−6-membered saturated, partially unsaturated, or aryl ring having 0-4 heteroatoms independently selected from nitrogen, oxygen, and sulfur.

In some embodiments, suitable substituents on a substitutable nitrogen are —R^{†}, —NR^{†}₂, —C(O)R^{†}, —C(O)OR^{†}, —C(O)C(O)R^{†}, —C(O)CH₂C(O)R^{†}, —S(O)₂Rt, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5−6-membered saturated, partially unsaturated, or aryl ring having 0-4 heteroatoms independently selected from nitrogen, oxygen, and sulfur, or, notwithstanding the definition above, two independent occurrences of RT, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●}is unsubstituted or where preceded by “halo” is substituted only with one or more halogens, and is independently C₁₋₄ aliphatic, —CH₂Ph, —O(CH₂)₀₋₁Ph, or a 5−6-membered saturated, partially unsaturated, or aryl ring having 0-4 heteroatoms independently selected from nitrogen, oxygen, and sulfur.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition. In some embodiments, a subject is a human.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Target polypeptide: A “target polypeptide”, as that term is used herein, is a polypeptide with which an agent interacts. In some embodiments, a target polypeptide is a beta-catenin polypeptide. In some embodiments, a target polypeptide comprises, consists essentially of, or is a binding site of beta-catenin polypeptide.

Target residue: A “target residue”, as that term is used herein, is a residue within a target polypeptide with which an agent is designed to interact. For example, an agent may be characterized by particular interaction motifs (e.g., aromatic groups as described herein) and/or residues (e.g., amino acid residues comprising aromatic groups as described herein) selected and arranged (by virtue of being presented on the selected scaffold) to be within a certain predetermined distance (or volume) of a target residue. In some embodiments, a target residue is or comprises an amino acid residue.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutic regimen: A “therapeutic regimen”, as that term is used herein, refers to a dosing regimen whose administration across a relevant population may be correlated with a desired or beneficial therapeutic outcome.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated” as used herein, means that a moiety has one or more units of unsaturation.

Unless otherwise specified, salts, such as pharmaceutically acceptable acid or base addition salts, stereoisomeric forms, and tautomeric forms, of provided compound are included.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

### Stapled Peptides

In some embodiments, a provided agent is or comprises a peptide. In some embodiments, a provided agent is a peptide. In some embodiments, a peptide is a stapled peptide. In some embodiments, a provided agent is a stapled peptide. In some embodiments, a peptide is a stitched peptide. In some embodiments, a provided agent is a stitched peptide. In some embodiments, a stitched peptide comprises two or more staples, wherein two staples are bonded to the same peptide backbone atom. Stapled peptides as described herein are typically peptides in which two or more amino acids of a peptide chain are linked through connection of two peptide backbone atoms of the amino acid residues and, as is understood by those skilled in the art, the connection is not through the peptide backbone between the linked amino acid residues. In some embodiments, a stapled peptide comprises one or more staples. A staple as described herein is a linker that can link one amino acid residue to another amino acid residue through bonding to a peptide backbone atom of each of the amino acid residues and, as is understood by those skilled in the art, the connection through a staple is not through the peptide backbone between the linked amino acid residues. In some embodiments, a staple bonds to the peptide backbone by replacing one or more hydrogen and/or substituents (e.g., side chains, O, S, etc.) on peptide backbone atoms (e.g., C, N, etc.). In some embodiments, side chains form portions of staples. In some embodiments, a staple is bonded to two carbon backbone atoms, e.g., two alpha carbon atoms. In some embodiments, a staple comprises C(R′)₂ or N(R′), either individually or as part of a large moiety, wherein R′ is R and is taken together with another group attached to a backbone atom which can be R (e.g., R^{a3}) and their intervening atoms to form a ring as described herein (e.g., when PyrS2 is stapled in various peptides).

As will be appreciated by those of ordinary skill in the art, a variety of peptide stapling technologies are available, including both hydrocarbon-stapling and non-hydrocarbon-stapling technologies, and can be utilized in accordance with the present disclosure. Various technologies for stapled and stitched peptides, including various staples and/or methods for manufacturing are available and may be utilized in accordance with the present disclosure, e.g., those described in WO 2019/051327 and WO 2020/041270, the staples of each of which are incorporated herein by reference.

In some embodiments, a peptide, e.g., a stapled peptide, is or comprise a helical structure. In some embodiments, a peptide is a stapled peptide.

In some embodiments, a staple is a hydrocarbon staple. In some embodiments, a staple as described herein is a non-hydrocarbon staple. In some embodiments, a non-hydrocarbon staple comprises one or more chain heteroatoms wherein a chain of a staple is the shortest covalent connection within the staple from one end of the staple to the other end of the staple. In some embodiments, a non-hydrocarbon staple is or comprises at least one sulfur atom derived from an amino acid residue of a polypeptide. In some embodiments, a non-hydrocarbon staple comprises two sulfur atom derived from two different amino acid residues of a polypeptide. In some embodiments, anon-hydrocarbon staple comprises two sulfur atoms derived from two different cysteine residues of a polypeptide. In some embodiments, a staple is a cysteine staple. In some embodiments, a staple is a non-cysteine staple. In some embodiments, a non-hydrocarbon staple is a carbamate staple and comprises a carbamate moiety (e.g., —N(R′)—C(O)—O—) in its chain. In some embodiments, a non-hydrocarbon staple is an amino staple and comprises an amino group (e.g., —N(R′)—) in its chain. In some embodiments, a non-hydrocarbon staple is an ester staple and comprises an ester moiety (—C(O)—O—) in its chain. In some embodiments, a non-hydrocarbon staple is an amide staple and comprises an amide moiety (—C(O)—N(R′)—) in its chain. In some embodiments, a non-hydrocarbon staple is a sulfonamide staple and comprises a sulfonamide moiety (—S(O)₂—N(R′)—) in its chain. In some embodiments, a non-hydrocarbon staple is an ether staple and comprises an ether moiety (—O—) in its chain. In some embodiments, an amino group in an amino staple, e.g., (—N(R′)—) is not bonded to a carbon atom that additionally forms a double bond with a heteroatom (e.g., —C(═O), —C(═S), —C(═N—R′), etc.) so that it is not part of another nitrogen-containing group such as amide, carbamate, etc. In some embodiments, R′ of a carbamate moiety, amino group, amide moiety, sulfonamide moiety, or ether moiety is R, and is taken together with an R group attached to a backbone (e.g., R^{a3} when it is R) and their intervening atoms to form a ring as described herein. In some embodiments, R′ of a carbamate moiety or amino group is R, and is taken together with an R group attached to a backbone (e.g., R^{a3} when it is R) and their intervening atoms to form a ring as described herein.

In some embodiments, a staple comprises one or more amino groups, e.g., —N(R′)—, wherein each R′ is independently as described herein. In some embodiments, —N(R′)— bonds to two carbon atoms. In some embodiments, —N(R′)— bonds to two carbon atoms, wherein neither of the two carbon atoms are bond to any heteroatoms through a double bond. In some embodiments, —N(R′)— bonds to two sp3 carbon atoms. In some embodiments, a staple comprises one or more —C(O)—N(R′)— groups, wherein each R′ is independently as described herein. In some embodiments, a staple comprises one or more carbamate groups, e.g., one or more —(O)—C(O)—N(R′)—, wherein each R′ is independently as described herein. In some embodiments, R′ is —H. In some embodiments, R′ is optionally substituted C₁₋₆ aliphatic. In some embodiments, R′ is optionally substituted C₁₋₆ alkyl. In some embodiments, R′ is C₁₋₆ aliphatic. In some embodiments, R′ is C₁₋₆ alkyl. In some embodiments, R′ is methyl.

In some embodiments, a stapled peptide comprise one or more staples. In some embodiments, a stapled peptide comprises one and no more than one staple. In some embodiments, a stapled peptide comprises two and no more than two staples. In some embodiments, two staples of a stapled peptide bond to a common backbone atom. In some embodiments, two staples of a stapled peptide bond to a common backbone atom which is an alpha carbon atom of an amino acid residue.

In some embodiments, peptides, e.g., staple peptides, of the present disclosure is or comprises a helix structure. As those skilled in the art will appreciate, helixes can have various lengths. In some embodiments, lengths of helixes range from 5 to 30 amino acid residues. In some embodiments, a length of a helix is 5, 6, 7, 8, 9, 10, 11, 12, 13, 14, 15, 16, 17, 18, 19, or 20, or more, amino acid residues. In some embodiments, a length of a helix is 6 amino acid residues. In some embodiments, a length of a helix is 8 amino acid residues. In some embodiments, a length of a helix is 10 amino acid residues. In some embodiments, a length of a helix is 12 amino acid residues. In some embodiments, a length of a helix is 14 amino acid residues. In some embodiments, a length of a helix is 16 amino acid residues. In some embodiments, a length of a helix is 17 amino acid residues. In some embodiments, a length of a helix is 18 amino acid residues. In some embodiments, a length of a helix is 19 amino acid residues. In some embodiments, a length of a helix is 20 amino acid residues.

Amino acids stapled together can have various number of amino acid residues in between, e.g., 1-20, 1-15, 1-10, 1, 2, 3, 4, 5, 6, 7, 8, 9, or 10, etc. In some embodiments, a staple is (i, i+4) which means there are three amino acid residues between the two amino acids (at positions i and i+4, respectively) that bond to the staple (at positions i+1, i+2, i+3, respectively). In some embodiments, a staple is (i, i+3). In some embodiments, a staple is (i, i+5). In some embodiments, a staple is (i, i+6). In some embodiments, a staple is (i, i+7). In some embodiments, a staple is (i, i+8). In some embodiments, a stapled peptide comprises two staples, one is (i, i+3) and the other is (i, i+7). In some embodiments, a stapled peptide comprises two staples, one is (i, i+3) and the other is (i, i+4). In some embodiments, a stapled peptide comprises two staples, one is (i, i+4) and the other is (i, i+7). In some embodiments, a stapled peptide comprises two staples, one is (i, i+3) and the other is (i, i+3). In some embodiments, a stapled peptide comprises two staples, one is (i, i+4) and the other is (i, i+4). In some embodiments, a stapled peptide comprises two staples, one is (i, i+7) and the other is (i, i+7). In some embodiments, the two staples are bonded to a common backbone atom, e.g., an alpha carbon atom of an amino acid residue.

In some embodiments, a stapled peptide comprises a staple which staple is L^{s}, wherein L^{s} is -L^{s1}-L^{s2}-L^{s3}-, each of L^{s1}, L^{s2}, and L^{s3} is independently L, wherein each L is independently as described in the present disclosure. In some embodiments, a provided staple is L^{s}.

In some embodiments, L^{s1} comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L^{s1} is -L′—N(R′)—, wherein L′ is optionally substituted bivalent C₁-C₁₉ aliphatic. In some embodiments, L^{s1} is -L′—N(CH₃)—, wherein L′ is optionally substituted bivalent C₁-C₁₉ aliphatic.

In some embodiments, R′ is optionally substituted C₁₋₆ alkyl. In some embodiments, R′ is C₁₋₆ alkyl. In some embodiments, R′ is methyl. In some embodiments, the peptide backbone atom to which L^{s1} is bonded is also bonded to R¹, and R′ and R1 are both R and are taken together with their intervene atoms to form an optionally substituted ring as described in the present disclosure. In some embodiments, a formed ring has no additional ring heteroatoms in addition to the nitrogen atom to which R′ is bonded. In some embodiments, a formed ring is 3-membered. In some embodiments, a formed ring is 4-membered. In some embodiments, a formed ring is 5-membered. In some embodiments, a formed ring is 6-membered.

In some embodiments, L′ is optionally substituted bivalent C₁-C₂₀ aliphatic. In some embodiments, L′ is optionally substituted bivalent C₁-C₁₉ aliphatic. In some embodiments, L′ is optionally substituted bivalent C₁-C₁₅ aliphatic. In some embodiments, L′ is optionally substituted bivalent C₁-C₁₀ aliphatic. In some embodiments, L′ is optionally substituted bivalent C₁-C₉ aliphatic. In some embodiments, L′ is optionally substituted bivalent C₁-C₅ aliphatic. In some embodiments, L′ is optionally substituted bivalent C₁-C₇ aliphatic. In some embodiments, L′ is optionally substituted bivalent C₁-C₆ aliphatic. In some embodiments, L′ is optionally substituted bivalent C₁-C₅ aliphatic. In some embodiments, L′ is optionally substituted bivalent C₁-C₄ aliphatic. In some embodiments, L′ is optionally substituted alkylene. In some embodiments, L′ is optionally substituted alkenylene. In some embodiments, L′ is unsubstituted alkylene. In some embodiments, L′ is —CH₂—. In some embodiments, L′ is —(CH₂)₂—. In some embodiments, L′ is —(CH₂)₃—. In some embodiments, L′ is —(CH₂)₄—. In some embodiments, L′ is —(CH₂)₅—. In some embodiments, L′ is —(CH₂)₆—. In some embodiments, L′ is —(CH₂)₇—. In some embodiments, L′ is —(CH₂)₈—. In some embodiments, L′ is bonded to a peptide backbone atom. In some embodiments, L′ is optionally substituted alkenylene. In some embodiments, L′ is unsubstituted alkenylene. In some embodiments, L′ is —CH₂—CH═CH—CH₂—.

In some embodiments, L′ is optionally substituted phenylene.

In some embodiments, L^{s1} comprises at least one —N(R′)C(O)—, wherein R′ is as described in the present disclosure. In some embodiments, L^{s1} is -L′—N(R′)C(O)—, wherein each of L′ and R′ is independently as described in the present disclosure. In some embodiments, L^{s1} is -L′—N(CH₃)C(O)—, wherein L′ is independently as described in the present disclosure.

In some embodiments, L^{s1} comprises at least one —C(O)O—. In some embodiments, L^{s1} comprises at least one —C(O)O—. In some embodiments, L^{s1} is -L′-C(O)O— or -L′—OC(O)—, wherein each L′ is independently as described in the present disclosure. In some embodiments, L^{s1} is -L′-C(O)O—, wherein each L′ is independently as described in the present disclosure. In some embodiments, L^{s1} is -L′—OC(O)—, wherein each L′ is independently as described in the present disclosure.

In some embodiments, L^{s1} comprises at least one —S(O)₂—N(R′)—, wherein R′ is as described in the present disclosure. In some embodiments, L^{s1} comprises at least one —S(O)₂—N(R′)—, wherein R′ is as described in the present disclosure. In some embodiments, L^{s1} is -L′—N(R′)—S(O)₂— or -L′-S(O)₂—N(R′)—, wherein each of L′ and R′ is independently as described in the present disclosure. In some embodiments, L^{s1} is -L′—N(R′)—S(O)₂—, wherein each of L′ and R′ is independently as described in the present disclosure. In some embodiments, L^{s1} is -L′-S(O)₂—N(R′)—, wherein each of L′ and R′ is independently as described in the present disclosure. In some embodiments, L^{s1} is -L′—N(CH₃)—S(O)₂— or -L′-S(O)₂—N(CH₃)—, wherein each L′ is independently as described in the present disclosure. In some embodiments, L^{s1} is -L′—N(CH₃)—S(O)₂—, wherein L′ is as described in the present disclosure. In some embodiments, L^{s1} is -L′-S(O)₂—N(CH₃)—, wherein L′ is as described in the present disclosure.

In some embodiments, L^{s1} comprises at least one —O—. In some embodiments, L^{s1} is -L′—O—, wherein L′ is independently as described in the present disclosure.

In some embodiments, L^{s1} is a covalent bond.

In some embodiments, L^{s1} is L′, wherein L′ is as described in the present disclosure.

In some embodiments, L^{s2} is L, wherein L is as described in the present disclosure. In some embodiments, L^{s2} is L′, wherein L′ is as described in the present disclosure. In some embodiments, L^{s2} comprises —CH₂—CH═CH—CH₂—. In some embodiments, L^{s2} is —CH₂—CH═CH—CH₂—. In some embodiments, L^{s2} comprises —(CH₂)₄—. In some embodiments, L^{s2} is —(CH₂)₄—.

In some embodiments, L^{s3} comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L^{s3} is -L′—N(R′)—, wherein L′ is optionally substituted bivalent C₁-C₁₉ aliphatic. In some embodiments, L^{s3} is -L′—N(CH₃)—, wherein L′ is optionally substituted bivalent C₁-C₁₉ aliphatic.

In some embodiments, L^{s3} comprises at least one —N(R′)C(O)—, wherein R′ is as described in the present disclosure. In some embodiments, L^{s3} is -L′—N(R′)C(O)—, wherein each of L′ and R′ is independently as described in the present disclosure. In some embodiments, L^{s3} is -L′—N(CH₃)C(O)—, wherein L′ is independently as described in the present disclosure.

In some embodiments, L^{s3} comprises at least one —C(O)O—. In some embodiments, L^{s3} comprises at least one —C(O)O—. In some embodiments, L^{s3} is -L′-C(O)O— or -L′—OC(O)—, wherein each L′ is independently as described in the present disclosure. In some embodiments, L^{s3} is -L′-C(O)O—, wherein each L′ is independently as described in the present disclosure. In some embodiments, L^{s3} is -L′—OC(O)—, wherein each L′ is independently as described in the present disclosure.

In some embodiments, L^{s3} comprises at least one —S(O)₂—N(R′)—, wherein R′ is as described in the present disclosure. In some embodiments, L^{s3} comprises at least one —S(O)₂—N(R′)—, wherein R′ is as described in the present disclosure. In some embodiments, L^{s3} is -L′—N(R′)—S(O)₂— or -L′-S(O)₂—N(R′)—, wherein each of L′ and R′ is independently as described in the present disclosure. In some embodiments, L^{s3} is -L′—N(R′)—S(O)₂—, wherein each of L′ and R′ is independently as described in the present disclosure. In some embodiments, L^{s3} is -L′-S(O)₂—N(R′)—, wherein each of L′ and R′ is independently as described in the present disclosure. In some embodiments, L^{s3} is -L′—N(CH₃)—S(O)₂— or -L′-S(O)₂—N(CH₃)—, wherein each L′ is independently as described in the present disclosure. In some embodiments, L^{s3} is -L′—N(CH₃)—S(O)₂—, wherein L′ is as described in the present disclosure. In some embodiments, L^{s3} is -L′-S(O)₂—N(CH₃)—, wherein L′ is as described in the present disclosure.

In some embodiments, L^{s3} comprises at least one —O—. In some embodiments, L^{s3} is -L′—O—, wherein L′ is independently as described in the present disclosure.

In some embodiments, L^{s3} is L′, wherein L′ is as described in the present disclosure. In some embodiments, L^{s3} is optionally substituted alkylene. In some embodiments, L^{s3} is unsubstituted alkylene.

In some embodiments, L^{s} comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L^{s} comprises at least one —N(R′)C(O)—, wherein R′ is as described in the present disclosure.

In some embodiments, L^{s}, L^{s1}, L^{s2}, and L^{s3} each independently and optionally comprise a R′ group, e.g., a R′ group in —C(R′)₂—, —N(R′)—, etc., and the R′ group is taken with a group (e.g., a group that can be R) attached to a backbone atom (e.g., R^{a1}, R^{a2}, R^{a3}, a R′ group of L^{a1} or L^{a2} (e.g., a R′ group in —C(R′)₂—, —N(R′)—, etc.), etc.) to form a double bond or an optionally substituted ring as two R groups can. In some embodiments, a formed ring is an optionally substituted 3-10 membered ring. In some embodiments, a formed ring is an optionally substituted 3-membered ring. In some embodiments, a formed ring is an optionally substituted 4-membered ring. In some embodiments, a formed ring is an optionally substituted 5-membered ring. In some embodiments, a formed ring is an optionally substituted 6-membered ring. In some embodiments, a formed ring is monocyclic. In some embodiments, a formed ring is saturated. In some embodiments, a formed ring is partially unsaturated. In some embodiments, a formed ring is aromatic. In some embodiments, a formed ring comprises one or more ring heteroatom (e.g., nitrogen). In some embodiments, a staple, or L^{s}, L^{s1}, L^{s2}, and/or L^{s3} comprises —N(R′)—, and the R′ is taken together with a group attached to a backbone atom to form an optionally substituted ring as described herein. In some embodiments, a staple, or L^{s}, L^{s1}, L^{s2}, and/or L^{s3} comprises —C(R′)₂—, and the R′ is taken together with a group attached to a backbone atom to form an optionally substituted ring as described herein.

In some embodiments, a staple, or L^{s}, L^{s1}, L^{s2}, and/or L^{s3} comprises portions of one or more amino acid side chains (e.g., a side chain other than its terminal ═CH₂).

As will be clear to those skilled in the art reading the present disclosure, the letter “L” is used to refer to a linker moiety as described herein; each L^{superscript}, (e.g., L^{a}, L^{s1}, L^{s2} L^{s3}, L^{s}, etc.) therefore is understood, in some embodiments, to be L, unless otherwise specified.

In some embodiments, L comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L is -L′—N(R′)—, wherein L′ is optionally substituted bivalent C₁-C₁₉ aliphatic. In some embodiments, L is -L′—N(CH₃)—, wherein L′ is optionally substituted bivalent C₁-C₁₉ aliphatic.

In some embodiments, L comprises at least one —N(R′)C(O)—, wherein R′ is as described in the present disclosure. In some embodiments, L is -L′—N(R′)C(O)—, wherein each of L′ and R′ is independently as described in the present disclosure. In some embodiments, L is -L′—N(CH₃)C(O)—, wherein L′ is independently as described in the present disclosure.

In some embodiments, L comprises at least one —C(O)O—. In some embodiments, L comprises at least one —C(O)O—. In some embodiments, L is -L′-C(O)O— or -L′—OC(O)—, wherein each L′ is independently as described in the present disclosure. In some embodiments, L is -L′-C(O)O—, wherein each L′ is independently as described in the present disclosure. In some embodiments, L is -L′—OC(O)—, wherein each L′ is independently as described in the present disclosure.

In some embodiments, L comprises at least one —S(O)₂—N(R′)—, wherein R′ is as described in the present disclosure. In some embodiments, L comprises at least one —S(O)₂—N(R′)—, wherein R′ is as described in the present disclosure. In some embodiments, L is -L′—N(R′)—S(O)₂— or -L′-S(O)₂—N(R′)—, wherein each of L′ and R′ is independently as described in the present disclosure. In some embodiments, L is -L′—N(R′)—S(O)₂—, wherein each of L′ and R′ is independently as described in the present disclosure. In some embodiments, L is -L′-S(O)₂—N(R′)—, wherein each of L′ and R′ is independently as described in the present disclosure. In some embodiments, L is -L′—N(CH₃)—S(O)₂— or -L′-S(O)₂—N(CH₃)—, wherein each L′ is independently as described in the present disclosure. In some embodiments, L is -L′—N(CH₃)—S(O)₂—, wherein L′ is as described in the present disclosure. In some embodiments, L is -L′-S(O)₂—N(CH₃)—, wherein L′ is as described in the present disclosure.

In some embodiments, L comprises at least one —O—. In some embodiments, L is -L′—O−, wherein L′ is independently as described in the present disclosure.

In some embodiments, L is L′, wherein L′ is as described in the present disclosure. In some embodiments, L is optionally substituted alkylene. In some embodiments, L is unsubstituted alkylene.

In some embodiments, L is optionally substituted bivalent C₁-C₂₅ aliphatic. In some embodiments, L is optionally substituted bivalent C₁-C₂₀ aliphatic. In some embodiments, L is optionally substituted bivalent C₁-C₁₅ aliphatic. In some embodiments, L is optionally substituted bivalent C₁-C₁₀ aliphatic. In some embodiments, L is optionally substituted bivalent C₁-C₉ aliphatic. In some embodiments, L is optionally substituted bivalent C₁-C₅ aliphatic. In some embodiments, L is optionally substituted bivalent C₁-C₇ aliphatic. In some embodiments, L is optionally substituted bivalent C₁-C₆ aliphatic. In some embodiments, L is optionally substituted bivalent C₁-C₅ aliphatic. In some embodiments, L is optionally substituted bivalent C₁-C₄ aliphatic. In some embodiments, L is optionally substituted alkylene. In some embodiments, L is optionally substituted alkenylene. In some embodiments, L is unsubstituted alkylene. In some embodiments, L is —CH₂—. In some embodiments, L is —(CH₂)₂—. In some embodiments, L is —(CH₂)₃—. In some embodiments, L is —(CH₂)₄—. In some embodiments, L is —(CH₂)₅—. In some embodiments, L is —(CH₂)₆—. In some embodiments, L is —(CH₂)₇—. In some embodiments, L is —(CH₂)₈—. In some embodiments, L is bonded to a peptide backbone atom. In some embodiments, L is optionally substituted alkenylene. In some embodiments, L is unsubstituted alkenylene. In some embodiments, L is —CH₂—CH═CH—CH₂—.

In some embodiments, one end of a staple is connected to an atom Aⁿ¹ of the peptide backbone, wherein A^{s1} is optionally substituted with R¹ and is an atom of an amino acid residue at amino acid position n¹ of the peptide from the N-terminus, and the other end is connected to an atom Aⁿ² of the peptide backbone, wherein Aⁿ² is optionally substituted with R² (in some embodiments, R′ and/or R² is R which can be hydrogen) and is an atom of an amino acid residue at amino acid position n² of the peptide from the N-terminus, wherein each of n¹ and n² is independently an integer, and n²=n¹+m, wherein m is 3-12.

In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, a staple is referred to a (i, i+m) staple.

In some embodiments, Aⁿ¹ is a carbon atom. In some embodiments, Aⁿ¹ is achiral. In some embodiments, Aⁿ¹ is chiral. In some embodiments, Aⁿ¹ is R. In some embodiments, Aⁿ¹ is S.

In some embodiments, Aⁿ² is a carbon atom. In some embodiments, Aⁿ² is achiral. In some embodiments, Aⁿ² is chiral. In some embodiments, Aⁿ² is R. In some embodiments, Aⁿ² is S.

In some embodiments, Aⁿ¹ is achiral and Aⁿ² is achiral. In some embodiments, Aⁿ¹ is achiral and Aⁿ² is R. In some embodiments, Aⁿ¹ is achiral and Aⁿ² is S. In some embodiments, Aⁿ¹ is R and Aⁿ² is achiral. In some embodiments, Aⁿ¹ is R and Aⁿ² is R. In some embodiments, Aⁿ¹ is R and Aⁿ² is S. In some embodiments, Aⁿ¹ is S and Aⁿ² is achiral. In some embodiments, Aⁿ¹ is S and Aⁿ² is R. In some embodiments, Aⁿ¹ is S and Aⁿ² is S.

In some embodiments, provided stereochemistry at staple-backbone connection points and/or combinations thereof, optionally together with one or more structural elements of provided peptide, e.g., staple chemistry (hydrocarbon, non-hydrocarbon), staple length, etc. can provide various benefits, such as improved preparation yield, purity, and/or selectivity, improved properties (e.g., improved solubility, improved stability, lowered toxicity, improved selectivities, etc.), improved activities, etc. In some embodiments, provided stereochemistry and/or stereochemistry combinations are different from those typically used, e.g., those of U.S. Pat. No. 9,617,309, US 2015-0225471, US 2016-0024153, US 2016-0215036, US 2016-0244494, WO 2017/062518, and provided one or more of benefits described in the present disclosure.

In some embodiments, a staple can be of various lengths, in some embodiments, as represent by the number of chain atoms of a staple. In some embodiments, a chain of a staple is the shortest covalent connection in the staple from a first end (connection point with a peptide backbone) of a staple to a second end of the staple, wherein the first end and the second end are connected to two different peptide backbone atoms. In some embodiments, a staple comprises 5-30 chain atoms, e.g., 5-20, 5-15, 5, 6, 7, 8, 9, or 10 to 11, 12, 13, 14, 15, 16, 17, 18, 19, 20, 21, 22, 23, 24, or 25 chain atoms. In some embodiments, a staple comprises 5 chain atoms. In some embodiments, a staple comprises 6 chain atoms. In some embodiments, a staple comprises 7 chain atoms. In some embodiments, a staple comprises 8 chain atoms. In some embodiments, a staple comprises 9 chain atoms. In some embodiments, a staple comprises 10 chain atoms. In some embodiments, a staple comprises 11 chain atoms. In some embodiments, a staple comprises 12 chain atoms. In some embodiments, a staple comprises 13 chain atoms. In some embodiments, a staple comprises 14 chain atoms. In some embodiments, a staple comprises 15 chain atoms. In some embodiments, a staple comprises 16 chain atoms. In some embodiments, a staple comprises 17 chain atoms. In some embodiments, a staple comprises 18 chain atoms. In some embodiments, a staple comprises 19 chain atoms. In some embodiments, a staple comprises 20 chain atoms. In some embodiments, a staple has a length of 5 chain atoms. In some embodiments, a staple has a length of 6 chain atoms. In some embodiments, a staple has a length of 7 chain atoms. In some embodiments, a staple has a length of 8 chain atoms. In some embodiments, a staple has a length of 9 chain atoms. In some embodiments, a staple has a length of 10 chain atoms. In some embodiments, a staple has a length of 11 chain atoms. In some embodiments, a staple has a length of 12 chain atoms. In some embodiments, a staple has a length of 13 chain atoms. In some embodiments, a staple has a length of 14 chain atoms. In some embodiments, a staple has a length of 15 chain atoms. In some embodiments, a staple has a length of 16 chain atoms. In some embodiments, a staple has a length of 17 chain atoms. In some embodiments, a staple has a length of 18 chain atoms. In some embodiments, a staple has a length of 19 chain atoms. In some embodiments, a staple has a length of 20 chain atoms. In some embodiments, a staple has a length of 8-15 chain atoms. In some embodiments, a staple has 8-12 chain atoms. In some embodiments, a staple has 9-12 chain atoms. In some embodiments, a staple has 9-10 chain atoms. In some embodiments, a staple has 8-10 chain atoms. In some embodiments, length of a staple can be adjusted according to the distance of the amino acid residues it connects, for example, a longer staple may be utilized for a (i, i+7) staple than a (i, i+4) or (i, i+3) staple. In some embodiments, a (i, i+3) staple has about 5-10, 5-8, e.g., about 5, 6, 7, 8, 9 or 10 chain atoms. In some embodiments, a (i, i+3) staple has 5 chain atoms. In some embodiments, a (i, i+3) staple has 6 chain atoms. In some embodiments, a (i, i+3) staple has 7 chain atoms. In some embodiments, a (i, i+3) staple has 8 chain atoms. In some embodiments, a (i, i+3) staple has 9 chain atoms. In some embodiments, a (i, i+3) staple has 10 chain atoms. In some embodiments, a (i, i+4) staple has about 5-12, 5-10, 7-12, 5-8, e.g., about 5, 6, 7, 8, 9, 10, 11 or 12 chain atoms. In some embodiments, a (i, i+4) staple has 5 chain atoms. In some embodiments, a (i, i+4) staple has 6 chain atoms. In some embodiments, a (i, i+4) staple has 7 chain atoms. In some embodiments, a (i, i+4) staple has 8 chain atoms. In some embodiments, a (i, i+4) staple has 9 chain atoms. In some embodiments, a (i, i+4) staple has 10 chain atoms. In some embodiments, a (i, i+4) staple has 11 chain atoms. In some embodiments, a (i, i+4) staple has 12 chain atoms. In some embodiments, a (i, i+7) staple has about 8-25, 10-25, 10-16, 12-15, e.g., about 8, 9, 10, 11, 12, 13, 14, 15, 16, 17, 18, 19, 20, 21, 22, 23, 24, or 25 chain atoms. In some embodiments, a (i, i+7) staple has 8 chain atoms. In some embodiments, a (i, i+7) staple has 9 chain atoms. In some embodiments, a (i, i+7) staple has 10 chain atoms. In some embodiments, a (i, i+7) staple has 11 chain atoms. In some embodiments, a (i, i+7) staple has 12 chain atoms. In some embodiments, a (i, i+7) staple has 13 chain atoms. In some embodiments, a (i, i+7) staple has 14 chain atoms. In some embodiments, a (i, i+7) staple has 15 chain atoms. In some embodiments, a (i, i+7) staple has 16 chain atoms. In some embodiments, a (i, i+7) staple has 17 chain atoms. In some embodiments, a (i, i+7) staple has 18 chain atoms. In some embodiments, a (i, i+7) staple has 19 chain atoms. In some embodiments, a (i, i+7) staple has 20 chain atoms. In some embodiments, a (i, i+7) staple has 21 chain atoms. In some embodiments, a (i, i+7) staple has 22 chain atoms. In some embodiments, a stapled peptide has two staples, each of which is independently such a (i, i+3), (i, i+4) or (i, i+7) staple. In some embodiments, a stapled peptide has such a (i, i+3) staple and such a (i, i+7) staple. In some embodiments, a stapled peptide has such a (i, i+4) staple and such a (i, i+7) staple.

Staple lengths may be otherwise described. For example, in some embodiments, staple lengths may be described as the total number of chain atoms and non-chain ring atoms, where a non-chain ring atom is an atom of the staple which forms a ring with one or more chain atoms but is not a chain atom in that it is not within the shortest covalent connection from a first end of the staple to a second end of the staple. In some embodiments, staples formed using Monomer A (which comprises an azetidine moiety), Monomer B (which comprises a pyrrolidine moiety), and/or Monomer C (which comprises a pyrrolidine moiety), etc., may comprise one or two non-chain ring atoms.

In some embodiments, a staple has no heteroatoms in its chain. In some embodiments, a staple comprises at least one heteroatom in its chain. In some embodiments, a staple comprises at least one nitrogen atom in its chain.

In some embodiments, a staple is L^{s}, wherein L^{s} is an optionally substituted, bivalent C₈₋₁₄ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₉₋₁₃ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₁₀₋₁₅ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₁₁₋₁₄ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is a (i, i+3) staple in that not including the two amino acid residues that are directly connected to the staple, there are two amino acid residues between the two amino acid residues that are directly connected to the staple. In some embodiments, a staple is a (i, i+4) staple in that not including the two amino acid residues that are directly connected to the staple, there are three amino acid residues between the two amino acid residues that are directly connected to the staple. In some embodiments, a staple is a (i, i+7) staple in that not including the two amino acid residues that are directly connected to the staple, there are six amino acid residues between the two amino acid residues that are directly connected to the staple.

In some embodiments, for each of L^{s}, L^{s1}, L^{s2}, and L^{s3}, any replacement of methylene units, if any, is replaced with —N(R′)—, —C(O)—N(R′)—, —N(R′)C(O)O—, —C(O)O—, —S(O)₂N(R′)—, or —O—. In some embodiments, for each of L^{s}, L^{s1}, L^{s2}, and L^{s3}, any replacement of methylene units, if any, is replaced with —N(R′)—, —N(R′)—C(O)—, or —N(R′)C(O)O—. In some embodiments, for each of L^{s}, L^{s1}, L^{s2}, and L^{s3}, any replacement of methylene units, if any, is replaced with —N(R′)— or —N(R′)C(O)O—. In some embodiments, for each of L^{s}, L^{s1}, L^{s2}, and L^{s3}, any replacement of methylene units, if any, is replaced with —N(R′)—. In some embodiments, for each of L^{s}, L^{s1}, L^{s2}, and L^{s3}, any replacement of methylene units, if any, is replaced with —N(R′)C(O)O—.

In some embodiments, a staple comprises a double bond. In some embodiments, a staple comprises a double bond may be formed by olefin metathesis of two olefins. In some embodiments, staples are formed by metathesis reactions, e.g., involving one or more double bonds in amino acid residues as described herein. In some embodiments, a first amino acid residue comprising an olefin (e.g., AA1-CH═CH₂) and a second amino acid residue comprising an olefin (e.g., AA2-CH═CH₂) are stapled (e.g., forming AA1-CH═CH-AA2, wherein AA1 and AA2 are typically linked through one or more amino acid residues). In some embodiments, an olefin, e.g., in a staple, is converted into —CHR′—CHR′—, wherein each R′ is independently as described herein. In some embodiments, R′ is R as described herein. In some embodiments, R′ is —H. In some embodiments, each R′ is —H. In some embodiments, R′ is —OR, wherein R is as described herein. In some embodiments, R′ is —OH. In some embodiments, R′ is —N(R)₂ wherein each R is independently as described herein. In some embodiments, R′ is —SR wherein R is as described herein. In some embodiments, R′ is R wherein R is optionally substituted aliphatic, e.g., C₁₋₁₀ aliphatic. In some embodiments, R′ is R wherein R is optionally substituted aliphatic, e.g., C₁₋₁₀ alkenyl. In some embodiments, R′ is R wherein R is optionally substituted aliphatic, e.g., C₁₋₁₀ alkynyl. In some embodiments, —CHR′—CHR′— is —CH₂—CH₂—. In some embodiments, each of the two olefins is independently of a side chain of an amino acid residue. In some embodiments, each olefin is independently a terminal olefin. In some embodiments, each olefin is independently a mono-substituted olefin.

In some embodiments, a suitable amino acid for stapling has structure of formula A-IL:

NH(R^{a1})-L^{a1}-C(-L^{a}-CH═CH₂)(R^{a3})-L^{a2}-COOH, A-II

or a salt thereof, wherein each variable is independently as described in the present disclosure.

In some embodiments, an amino acid of formula A-I is a compound having the structure of formula A-III:

NH(R^{a1})—C(-L^{a}-CH═CH₂)(R^{a3})—COOH, A-III

or a salt thereof, wherein each variable is independently as described in the present disclosure.

In some embodiments, an amino acid of formula A-I or a salt thereof has structure of formula A-IV:

NH(R^{a1})-L^{a1}-C(-L^{a}-COOH)(R^{a3})-L^{a2}-COOH, A-IV

or a salt thereof, wherein each variable is independently as described in the present disclosure. In some embodiments, an amino acid suitable for stapling has the structure of formula A-IV or a salt thereof, wherein each variable is independently as described in the present disclosure.

In some embodiments, an amino acid has structure of formula A-V:

NH(R^{a1})-L^{a1}-C(-L^{a}-R^{SP1})(R^{a3})-L^{a2}-COOH, A-V

or a salt thereof, wherein each variable is independently as described in the present disclosure. In some embodiments, an amino acid suitable for stapling has the structure of formula A-V or a salt thereof, wherein each variable is independently as described in the present disclosure.

In some embodiments, an amino acid for stapling has structure of formula A-VI:

NH(R^{a1})-L^{a1}-C(-L^{a}-R^{SP1})(-L^{a}-R^{SP2})-L^{a2}-COOH, A-VI

or a salt thereof, wherein each variable is independently as described in the present disclosure. In some embodiments, an amino acid suitable for stapling has the structure of formula A-VI or a salt thereof, wherein each variable is independently as described in the present disclosure.

As used herein, each of R^{SP1} and R^{SP2} independently comprises a reactive group. In some embodiments, each of R^{SP1} and R^{SP2} is independently a reactive group. In some embodiments, a reactive group is optionally substituted —CH═CH₂. In some embodiments, a reactive group is —CH═CH₂. In some embodiments, a reactive group is an amino group, e.g., —NHR, wherein R is as described herein. In some embodiments, a reactive group is an acid group. In some embodiments, a reactive group is —COOH or an activated form thereof. In some embodiments, a reactive group is for a cycloaddition reaction (e.g., [3+2], [4+2], etc.), e.g., an alkene, an alkyne, a diene, a 1,3-dipole (e.g., —N₃), etc. In some embodiments, a reactive group is optionally substituted —C≡CH. In some embodiments, a reactive group is —C≡CH. In some embodiments, a reactive group is —N₃.

In some embodiments, R^{SP1} or R^{SP2} of a first amino acid residue and R^{SP1} or R^{SP2} of a second amino acid residue can react with each other so that the two amino acid residues are connected with a staple. In some embodiments, a reactive is olefin metathesis between two olefin, e.g., two —CH═CH₂. In some embodiments, a reaction is amidation and one reactive group is an amino group, e.g., —NHR wherein R is as described herein (e.g., in some embodiments, R is —H; in some embodiments, R is optionally substituted C₁₋₆ aliphatic), and the other is an acid group (e.g., —COOH) or an activated form thereof. In some embodiments, a reaction is a cycloaddition reaction, e.g., [4+2], [3+2], etc. In some embodiments, a first and a second reactive groups are two reactive groups suitable for a cycloaddition reaction. In some embodiments, a reaction is a click reaction. In some embodiments, one reaction group is or comprises —N₃, and the other is or comprises an alkyne, e.g., a terminal alkyne or a activated/strained alkyne. In some embodiments, the other is or comprises —C≡CH.

In some embodiments, R^{SP1} or R^{SP2} of a first amino acid residue and R^{SP1} or R^{SP2} of a second amino acid residue can react with a reagent so that the two are connected to form a staple. In some embodiments, a reagent comprises two reactive groups, one of which reacts with R^{SP1} or R^{SP2} of a first amino acid residue, and the other reacts with R^{SP1} or R^{SP2} of a first amino acid residue. In some embodiments, R^{SP1} or R^{SP2} of both amino acid residues are the same or the same type, e.g., both are amino groups, and the two reactive groups of a linking reagent are also the same, e.g., both are acid groups such as —COOH or activated form thereof. In some embodiments, R^{SP1} or R^{SP2} of both amino acid residues are both acid groups, e.g., —COOH or activated form thereof, and both reactive groups of a linking agent are amino groups. In some embodiments, R^{SP1} or R^{SP2} of both amino acid residues are both nucleophilic groups, e.g., —SH, and both reactive groups of a linking reagent are electrophilic (e.g., carbon attached to leaving groups such as —Br, —I, etc.).

In some embodiments, R^{SP1} and R^{SP2} are the same. In some embodiments, R^{SP1} and R^{SP2} are different. In some embodiments, R^{SP1} is or comprises —CH═CH₂. In some embodiments, R^{SP1} is or comprises —COOH. In some embodiments, R^{SP1} is or comprises an amino group. In some embodiments, R^{SP1} is or comprises —NHR. In some embodiments, R is hydrogen or optionally substituted C₁₋₆ aliphatic. In some embodiments, R^{SP1} is or comprises —NH₂. In some embodiments, R^{SP1} is or comprises —N₃. In some embodiments, R^{SP2} is or comprises —CH═CH₂. In some embodiments, R^{SP2} is or comprises —COOH. In some embodiments, R^{SP2} is or comprises an amino group. In some embodiments, R^{SP2} is or comprises —NHR. In some embodiments, R is hydrogen or optionally substituted C₁₋₆ aliphatic. In some embodiments, R^{SP2} is or comprises —NH₂. In some embodiments, R^{SP2} is or comprises —N₃.

In some embodiments, each amino acid residue of a pair of amino acid residues is independently a residue of an amino acid of formula A-II or A-III or a salt thereof. In some embodiments, such a pair of amino acid residues is stapled, e.g., through olefin metathesis. In some embodiments, a staple has the structure of -L^{a}-CH═CH-L^{a}-, wherein each variable is independently as described herein. In some embodiments, olefin in a staple is reduced. In some embodiments. In some embodiments, a staple has the structure of -L^{a}-CH₂—CH₂-L^{a}-, wherein each variable is independently as described herein. In some embodiments, one L^{a} is L^{s1} as described herein, and one L^{a} is L^{s3} as described herein.

In some embodiments, each amino acid residue of a pair of amino acid residues is independently a residue of an amino acid of formula A-II or A-III or a salt thereof. In some embodiments, such a pair of amino acid residues is stapled, e.g., through olefin metathesis. In some embodiments, a staple has the structure of -L^{a}-CH═CH-L^{a}-, wherein each variable is independently as described herein. In some embodiments, olefin in a staple is reduced. In some embodiments. In some embodiments, a staple has the structure of -L^{a}-CH₂—CH₂-L^{a}-, wherein each variable is independently as described herein. In some embodiments, one L^{a} is L^{s1} as described herein, and one L^{a} is L^{s3} as described herein.

In some embodiments, two amino acid residues, e.g., of amino acids independently of formula A-I or a salt of, connected by a staple have the structure of —N(R^{a1})-L^{a1}-C(-L^{s}-R^{AA})(R^{a3})-L^{a2}-CO—, wherein each variable is independently as described herein, and R^{AA} is an amino acid residue. In some embodiments, two amino acid residues, e.g., of amino acids independently of formula A-I or a salt of, connected by a staple have the structure of —N(-L^{s}-R^{AA})-L^{a1}-C(R^{a2})(R^{a3})-L^{a2}-CO—, wherein each variable is independently as described herein, and R^{AA} is an amino acid residue. In some embodiments, two amino acid residues, e.g., of amino acids independently of formula A-I or a salt of, connected by a staple have the structure of R^{a1}—N(-L^{s}-R^{AA})-L^{a1}-C(R^{a2})(R^{a3})-L^{a2}-CO—, wherein each variable is independently as described herein, and R^{AA} is an amino acid residue. In some embodiments, three amino acid residues, e.g., of amino acids independently of formula A-I or a salt of, connected by two staples have the structure of R^{a1}—N(-L^{s}-R^{AA})-L^{a1}-C(-L^{s}-R^{AA})(R^{a3})-L^{a2}-CO—, wherein each variable is independently as described herein, and R^{AA} is an amino acid residue. In some embodiments, three amino acid residues, e.g., of amino acids independently of formula A-I or a salt of, connected by two staples have the structure of —N(-L^{s}-R^{AA})-L^{a1}-C(-L^{s}-R^{AA})(R^{a3})-L^{a2}-CO—, wherein each variable is independently as described herein, and R^{AA} is an amino acid residue. In some embodiments, three amino acid residues, e.g., of amino acids independently of formula A-I or a salt of, connected by two staples (e.g., X⁴ stapled with both X¹ and X¹⁴) have the structure of —N(R^{a1})-L^{a1}-C(-L^{s}-R^{AA})(-L^{s}-R^{AA})-L^{a2}-CO—, wherein each variable is independently as described herein, and R^{AA} is an amino acid residue. In some embodiments, each R^{AA} is independently a residue of an amino acid of formula A-I, A-II, A-III, A-IV, A-V, A-VI, etc. or a salt thereof. In some embodiments, R^{AA} is —C(R^{a3})[-L^{a1}-N(R^{a1})—](-L^{a2}-CO—), wherein each variable is independently as described herein. In some embodiments, R^{AA} is —C(R^{a3})[N(R^{a1})—](—CO—), wherein each variable is independently as described herein. In some embodiments, each R^{AA} is independently —N(—)[-L^{a1}-C(R^{a2})(R^{a3})-L^{a2}-CO—], wherein each variable is independently as described herein, wherein —C(—)(R^{a3})— is bonded to a staple. In some embodiments, each R^{AA} is independently —N(—)[—C(R^{a2})(R^{a3})CO—], wherein each variable is independently as described herein, wherein —C(—)(R^{a3})— is bonded to a staple. In some embodiments, each R^{AA} is independently R^{a1}—N(—)[-L^{a1}-C(R^{a2})(R^{a3})-L^{a2}-CO—], wherein each variable is independently as described herein, wherein —C(—)(R^{a3})— is bonded to a staple. In some embodiments, each R^{AA} is independently R^{a1}—N(—)[—C(R^{a2})(R^{a3})—CO—], wherein each variable is independently as described herein, wherein —C(—)(R^{a3})— is bonded to a staple.

Various staples, e.g., L^{s}, are as described herein. In some embodiments, L^{s} is -L^{s1}-L^{s2}-L^{s3}- as described herein. In some embodiments, L^{s1} is L^{a} as described herein. In some embodiments, L^{s3} is L^{a} as described herein. In some embodiments, L^{s1} is L^{a} of a first of two stapled amino acid residues. In some embodiments, L^{s2} is L^{a} of a second of two stapled amino acid residues. In some embodiments, L^{s2} is or comprises a double bond. In some embodiments, L^{s2} is or comprises —CH═CH—. In some embodiments, L^{s2} is or comprises optionally substituted —CH₂—CH₂—. In some embodiments, L^{s2} is or comprises —CH₂—CH₂—. In some embodiments, L^{s2} is or comprises —C(O)N(R′)— (e.g., a staple formed by two amino acid residues one of which has a R^{SP1} group that is or comprises an amino group and the other of which has a R^{SP2} group that is or comprises —COOH). In some embodiments, L^{s2} is or comprises —C(O)NH—. In some embodiments, each of L^{s1} and L^{s3} is independently optionally substituted linear or branched C₁₋₁₀ hydrocarbon chain. In some embodiments, each of L^{s1} and L^{s3} is independently —(CH₂)ₙ—, wherein n is 1-10. In some embodiments, L^{s1} is —CH₂—. In some embodiments, L^{s3} is —(CH₂)₃—.

In some embodiments, L^{s} is —CH₂—CH═CH—(CH₂)₃—. In some embodiments, L^{s} is —(CH₂)₆—.

In some embodiments, L^{s} is —(CH₂)₂—C(O)NH—(CH₂)₄—.

In some embodiments, L^{s} is bonded to two backbone carbon atoms. In some embodiments, L^{s} is bonded to two alpha carbon atoms of two stapled amino acid residues. In some embodiments, L^{s} is bonded to a backbone nitrogen atom and a backbone carbon atom (e.g., an alpha carbon).

In some embodiments, L^{a} comprises at least one —N(R′)— wherein R′ is independently as described in the present disclosure. In some embodiments, L^{a} comprises -L^{am1}-N(R′)— wherein R′ is independently as described in the present disclosure, and L^{am1} is as described herein. In some embodiments, L^{a} is or comprises -L^{am1}-N(R′)-L^{am2}-, wherein each of L^{am1}, R′, and L^{am2} is independently as described herein. In some embodiments, R′ is optionally substituted C₁₋₆ aliphatic. In some embodiments, R′ is methyl. In some embodiments, R′ is taken together with R^{a3} to form an optionally substituted ring as described herein. In some embodiments, a formed ring is a 3-10 membered monocyclic saturated ring as described herein. In some embodiments, a formed ring has no additional heteroatom ring atom in addition to the nitrogen of —N(R′)—. In some embodiments, a formed ring is 3-membered. In some embodiments, a formed ring is 4-membered. In some embodiments, a formed ring is 5-membered. In some embodiments, a formed ring is 6-membered.

In some embodiments, L^{a} comprises at least one —C(R′)₂— wherein each R′ is independently as described in the present disclosure. In some embodiments, L^{a} comprises -L^{am1}-C(R′)₂— wherein R′ is independently as described in the present disclosure, and L^{am1} is as described herein. In some embodiments, L^{a} is or comprises -L^{am1}-C(R′)₂-L^{am2}-, wherein each of L^{am1}, R′, and L^{am2} is independently as described herein. In some embodiments, R′ is —H. In some embodiments, —C(R′)₂— is optionally substituted —CH₂—. In some embodiments, —C(R′)₂— is —CH₂—. In some embodiments, one R′ is taken together with R^{a3} to form an optionally substituted ring as described herein. In some embodiments, a formed ring is a 3-10 membered monocyclic saturated ring as described herein. In some embodiments, a formed ring has no additional heteroatom ring atom in addition to the nitrogen of —N(R′)—. In some embodiments, a formed ring is 3-membered. In some embodiments, a formed ring is 4-membered. In some embodiments, a formed ring is 5-membered. In some embodiments, a formed ring is 6-membered.

As described herein, each of L^{am1} and L^{am2} is independently L^{am} as described herein. As described herein, L^{am} is a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{am} is a covalent bond. In some embodiments, L^{am} is an optionally substituted bivalent C₁-C₁₀ aliphatic group. In some embodiments, L^{am} is an optionally substituted bivalent linear C₁-C₁₀ aliphatic group. In some embodiments, L^{am} is optionally substituted C₁₋₁₀ alkylene. In some embodiments, L^{am} is C₁₋₁₀ alkylene. In some embodiments, L^{am} is optionally substituted linear C₁₋₁₀ alkylene. In some embodiments, L^{am} is optionally substituted —CH₂—. In some embodiments, L^{am} is —CH₂—.

In some embodiments, L^{a}mL is a covalent bond. In some embodiments, L^{am1} is an optionally substituted bivalent C₁-C₁₀ aliphatic group. In some embodiments, L^{am1} is an optionally substituted bivalent linear C₁-C₁₀ aliphatic group. In some embodiments, L^{am1} is optionally substituted C₁₋₁₀ alkylene. In some embodiments, L^{am1} is C₁₋₁₀ alkylene. In some embodiments, L^{a1} is optionally substituted linear C₁-10 alkylene. In some embodiments, L^{am1} is optionally substituted —CH₂—. In some embodiments, L^{am1} is —CH₂—. In some embodiments, L^{am1} is bonded to a backbone atom. In some embodiments, L^{am1} is bonded to an alpha-carbon of an amino acid.

In some embodiments, L^{am2} is a covalent bond. In some embodiments, L^{am2} is an optionally substituted bivalent C₁-C₁₀ aliphatic group. In some embodiments, L^{am2} is an optionally substituted bivalent linear C₁-C₁₀ aliphatic group. In some embodiments, L^{am2} is optionally substituted C₁₋₁₀ alkylene. In some embodiments, L^{am2} is C₁₋₁₀ alkylene. In some embodiments, L^{am2} is optionally substituted linear C₁₋₁₀ alkylene. In some embodiments, L^{am2} is optionally substituted —CH₂—. In some embodiments, L^{am2} is —CH₂—. In some embodiments, L^{am2} is or comprises —C(O)—. In some embodiments, —C(O)— is bonded to a nitrogen atom. In some embodiments, L^{am2} is or comprises —S(O)₂—. In some embodiments, —S(O)₂— is bonded to a nitrogen atom. In some embodiments, L^{am2} is or comprises —O—. In some embodiments, L^{am2} is or comprises —C(O)—O—. In some embodiments, —C(O)—O— is bonded to a nitrogen atom. In some embodiments, L^{am2} is bonded to a nitrogen atom, and it comprises a —C(O)— group which is bonded to the nitrogen atom. In some embodiments, L^{am2} is bonded to a nitrogen atom, and it comprises a —C(O)—O— group which is bonded to the nitrogen atom. In some embodiments, L^{am2} is or comprises —C(O)—O—CH₂—, wherein the —CH₂— is optionally substituted. In some embodiments, L^{am2} is —C(O)—O—CH₂—.

In some embodiments, L^{a} is L^{s1} as described herein. In some embodiments, L^{a} is L^{s2} as described herein.

In some embodiments, R^{a3} is -L^{a}-CH═CH₂, wherein L^{a} is independently as described herein. In some embodiments, each of R^{a2} and R^{a3} independently comprises a double bond, e.g., a terminal olefin which can be optionally and independently stapled with another residue comprising an olefin. In some embodiments, each of R^{a2} and R^{a3} are independently -L^{a}-CH═CH₂. In some embodiments, an amino acid are stapled with two amino acid residues independently through R^{a2} and R^{a3}. In some embodiments, such an amino acid is B5.

In some embodiments, an amino acid is selected from Tables A-I, A-II, A-III (may be presented as Fmoc-protected). As appreciated by those skilled in the art, among other things, when incorporated into peptides, Fmoc-protected amino groups and carboxyl groups may independently form amide connections with other amino acid residues (or N- or C-terminus capping groups, or exist as N- or C-terminus amino or carboxyl groups). Olefins, including those in Alloc groups, may be utilized to form staples through olefin metathesis. Staples comprising olefins may be further modified, e.g., through hydrogenation to convert olefin double bonds into single bonds, and/or through CO₂ extrusion to convert carbamate moieties (e.g., —O—(CO)—N(R′)—) into amine moieties (e.g., —N(R′)—). In some embodiments, an agent is or comprises a stapled peptide (e.g., a stapled peptide described according to Table E3) or a salt thereof, in which stapled peptide each double bond is converted into a single bond. In some embodiments, a conversion is achieved through hydrogenation which adds a —H to each olefin carbon atom. In some embodiments, an olefin double bond is replaced with —CHR′—CHR′—, wherein each R′ is independently as described herein. In some embodiments, R′ is R as described herein. In some embodiments, R′ is —H. In some embodiments, each R′ is —H. In some embodiments, R′ is —OR, wherein R is as described herein. In some embodiments, R′ is —OH. In some embodiments, R′ is —N(R)₂ wherein each R is independently as described herein. In some embodiments, R′ is —SR wherein R is as described herein. In some embodiments, R′ is R wherein R is optionally substituted aliphatic, e.g., C₁₋₁₀ aliphatic. In some embodiments, R′ is R wherein R is optionally substituted aliphatic, e.g., C₁₋₁₀ alkenyl. In some embodiments, R′ is R wherein R is optionally substituted aliphatic, e.g., C₁₋₁₀ alkynyl. In some embodiments, —CHR′—CHR′— is —CH₂—CH₂—.

TABLE A-I
Exemplary amino acids (Fmoc-Protected).

| | | |
|---|---|---|
| Monomer A (M_{A}) | Monomer B (M_{B}) | Monomer C (M_{C}) |
| 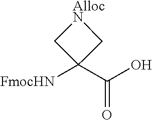 | 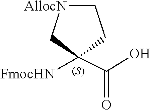 | 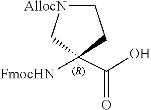 |

TABLE A-II
Exemplary amino acids (Fmoc-Protected).

| | | |
|---|---|---|
| Monomer D (M_{D}) | Monomer E (M_{E}) | Monomer F (M_{F}) |
| 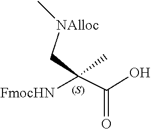 | 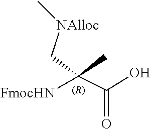 | 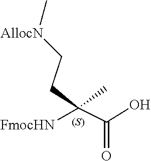 |
| Monomer G (M_{G}) | Monomer H (M_{H}) | Monomer I (M_{I}) |
| 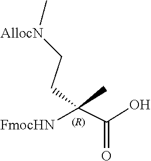 | 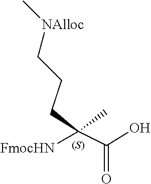 | 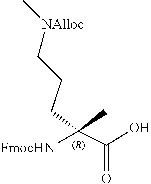 |

TABLE A-III
Exemplary amino acids (Fmoc-Protected).
S₃

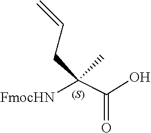

R₃

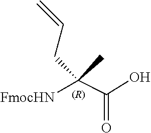

S₄

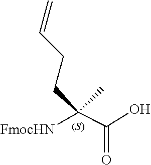

R₄

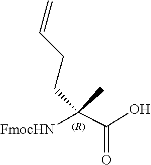

S₅

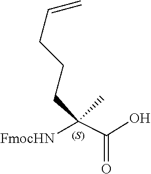

R₅

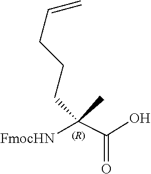

B₅

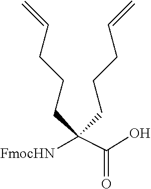

S₆

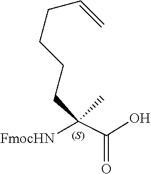

R₆

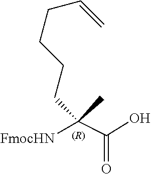

S₇

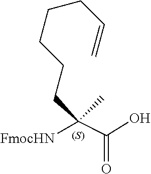

R₇

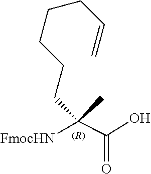

S₈

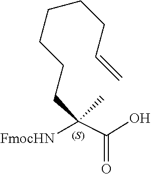

R8

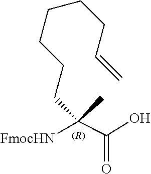

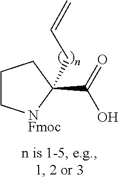

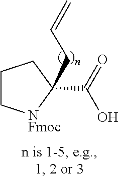

PL3

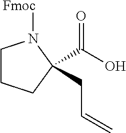

PyrS

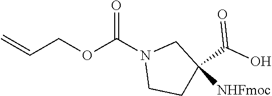

PyrS1

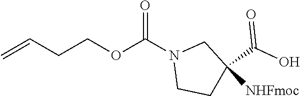

PyrS2

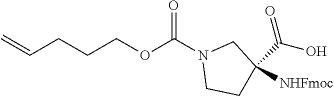

PyrS3

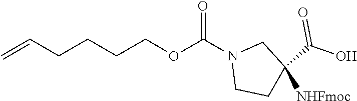

RdN

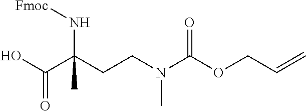

ReN

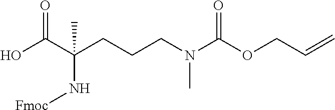

RgN

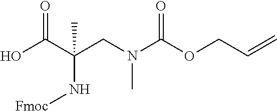

S10

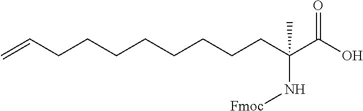

SdN

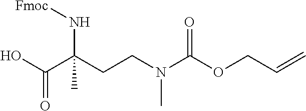

SeN

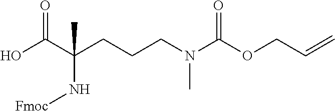

SgN

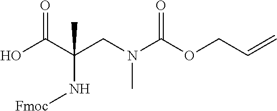

In some embodiments, an amino acid is an alpha-amino acid. In some embodiments, an amino acid is an L-amino acid. In some embodiments, an amino acid is a D-amino acid. In some embodiments, the alpha-carbon of an amino acid is achiral. In some embodiments, an amino acid is a beta-amino acid. In some embodiments, an amino acid is a gamma-amino acid.

In some embodiments, a provided amino acid sequence contains two or more amino acid residues whose side chains are linked together to form one or more staples. In some embodiments, a provided amino acid sequence contains two or more amino acid residues, each of which independently has a side chain comprising an olefin. In some embodiments, a provided amino acid sequence contains two or more amino acid residues, each of which independently has a side chain comprising a terminal olefin. In some embodiments, a provided amino acid sequence contains two and no more than two amino acid residues, each of which independently has a side chain comprising an olefin. In some embodiments, a provided amino acid sequence contains two and no more than two amino acid residues, each of which independently has a side chain comprising a terminal olefin. In some embodiments, a provided amino acid sequence comprises at least one residue of an amino acid that comprises an olefin and a nitrogen atom other than the nitrogen atom of its amino group. In some embodiments, a provided amino acid sequence comprises at least one residue of an amino acid that comprises a terminal olefin and a nitrogen atom other than the nitrogen atom of its amino group. In some embodiments, a provided amino acid sequence comprises at least one residue of an amino acid that has a side chain than comprises a terminal olefin and a nitrogen atom. In some embodiments, a provided amino acid sequence comprises at least one residue of an amino acid of formula A-I, wherein R^{a2} comprising an olefin and a —N(R′)— moiety, wherein R′ is as described in the present disclosure (including, in some embodiments, optionally taken together with R^{a3} and their intervening atoms to form an optionally substituted ring as described in the present disclosure). In some embodiments, R^{a2} comprising a terminal olefin and a —N(R′)— moiety wherein R′ is as described in the present disclosure. In some embodiments, a provided amino acid sequence comprises at least one residue of an amino acid selected from Table A-I. In some embodiments, a provided amino acid sequence comprises at least one residue of an amino acid selected from Table A-II. In some embodiments, a provided amino acid sequence comprises at least one residue of an amino acid selected from Table A-III. In some embodiments, two olefins from two side chains are linked together through olefin metathesis to form a staple. In some embodiments, a staple is preferably formed by side chains of amino acid residues that are not at the corresponding positions of a target of interest. In some embodiments, a formed staple does not disrupt interaction between the peptide and a target of interest.

In some embodiments, a provided staple is a hydrocarbon staple. In some embodiments, a hydrocarbon staple comprises no chain heteroatoms wherein a chain of a staple is the shortest covalent connection within the staple from one end of the staple to the other end of the staple.

In some embodiments, an olefin in a staple is a Z-olefin. In some embodiments, an olefin in a staple in an E-olefin. In some embodiments, a provided composition comprises stapled peptides comprising a staple that contains a Z-olefin and stapled peptides comprising a staple that contains an E-olefin. In some embodiments, a provided composition comprises stapled peptides comprising a staple that contains a Z-olefin. In some embodiments, a provided composition comprises stapled peptides comprising a staple that contains an E-olefin. In some embodiments, otherwise identical stapled peptides that differ only in the E Z configuration of staple olefin demonstrate different properties and/or activities as demonstrated herein. In some embodiments, stapled peptides with E-olefin in a staple may provide certain desirable properties and/or activities given the context. In some embodiments, stapled peptides with Z-olefin in a staple may provide certain desirable properties and/or activities given the context.

In some embodiments, the present disclosure provides compositions comprising stapled peptides. In some embodiments, a composition comprises one and only one stereoisomer of a stapled peptide (e.g., E or Z isomer, and/or a single diastereomer/enantiomer with respect to a chiral center, etc.). In some embodiments, a composition comprises two or more stereoisomers (e.g., both E and Z isomers of one or more double bonds, and/or one or more diastereomers/enantiomers with respect to a chiral center, etc.). In some embodiments, a composition corresponds to a single peak in a chromatographic separation, e.g., HPLC. In some embodiments, a peak comprises one and only one stereoisomers. In some embodiments, a peak comprises two or more stereoisomers.

In some embodiments, two staples may be bonded to the same atom of the peptide backbone, forming a stitched peptide.

In some embodiments, a staple is pro-lock wherein one end of the staple is bonded to the alpha-carbon of a proline residue.

In some embodiments, a staple is a staple illustrated below in Tables S-1, S-2, S-3, S-4 and S-5 (with exemplary peptide backbone illustrated for clarity (can be applied to other peptide backbone), each X independently being an amino acid residue). In some embodiments, a staple is a staple in Table S-6 (with amino acid residues bonded to staples illustrated). In some embodiments, the olefin is Z. In some embodiments, the olefin is E. In some embodiments, an (i, i+3) staple is selected from Table S-1. In some embodiments, an (i, i+3) staple is selected from Table S-2. Those skilled in the art reading the present disclosure will appreciate that when staples in Table S-1 and Table S-2 are utilized for (i, i+3), “X₃” in those tables would be “X₂” (i.e., two amino acid residues instead of three amino acid residues). In some embodiments, an (i, i+4) staple is selected from Table S-1. In some embodiments, an (i, i+4) staple is selected from Table S-2. In some embodiments, an (i, i+7) staple is selected from Table S-3. In some embodiments, an (i, i+7) staple is selected from Table S-4.

TABLE S-1
Exemplary staples.

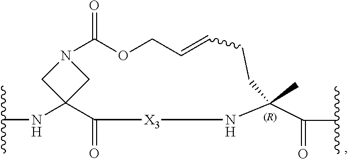

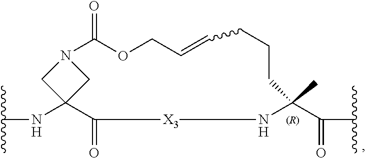

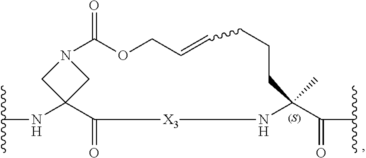

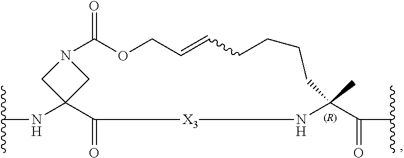

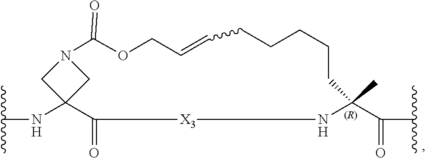

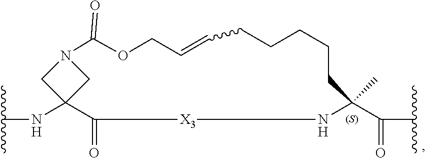

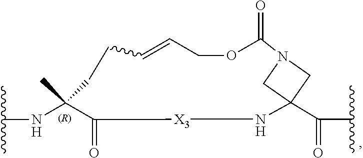

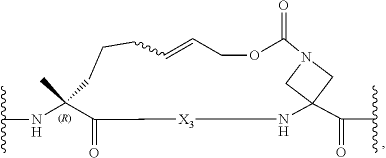

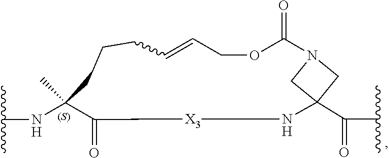

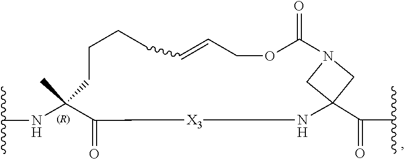

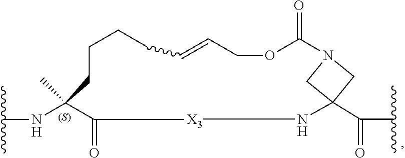

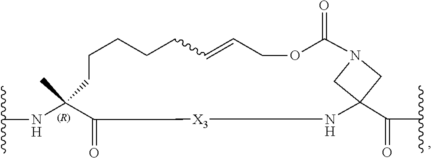

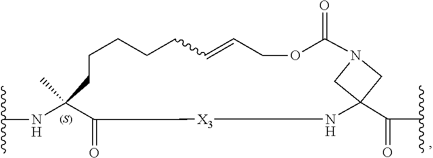

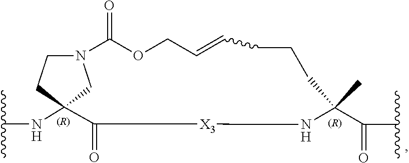

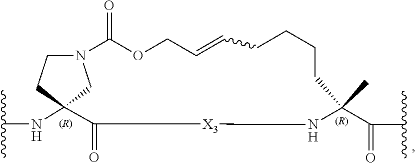

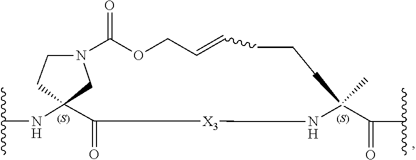

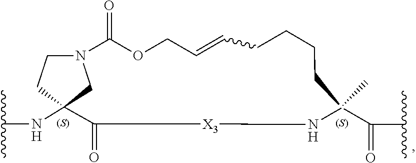

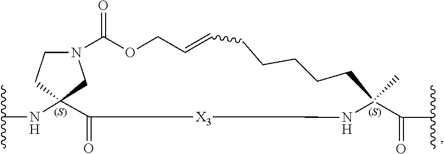

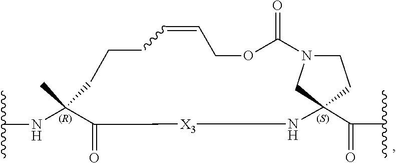

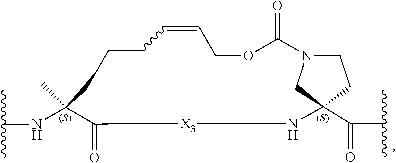

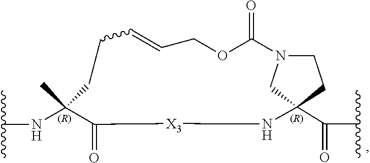

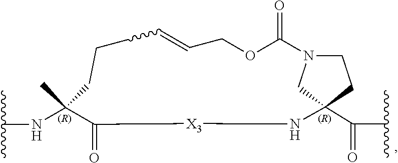

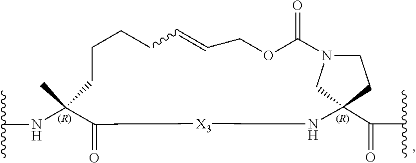

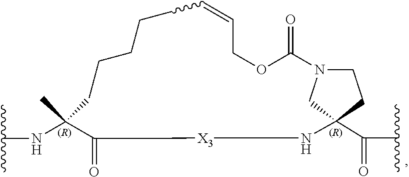

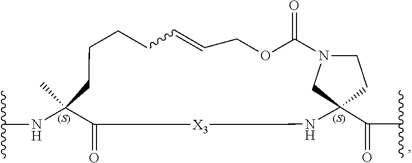

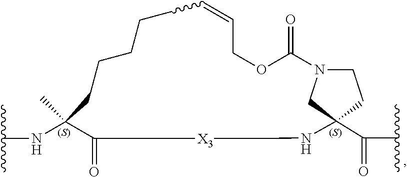

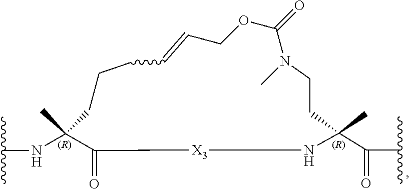

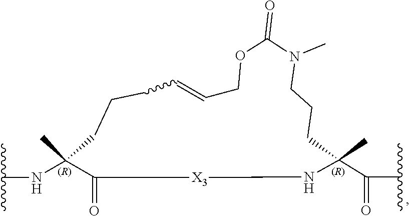

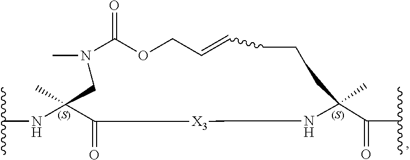

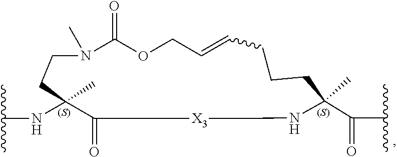

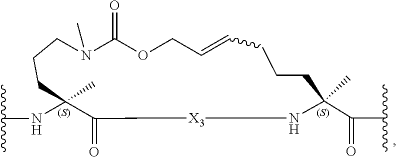

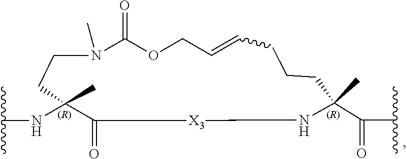

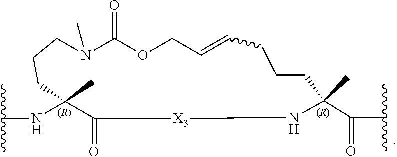

TABLE S-2
Exemplary staples.

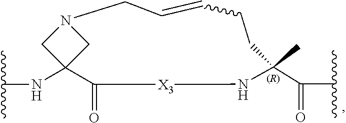

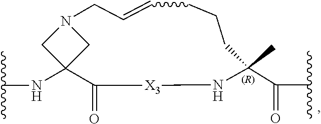

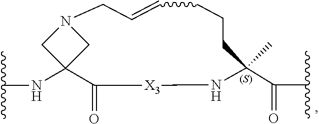

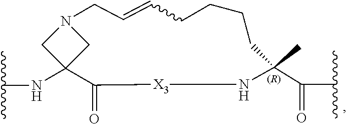

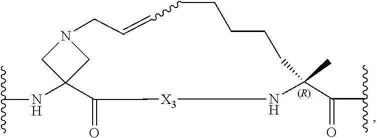

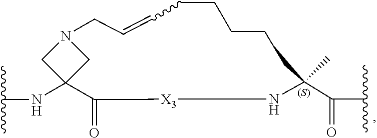

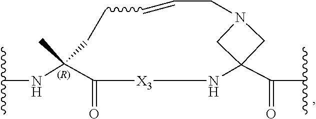

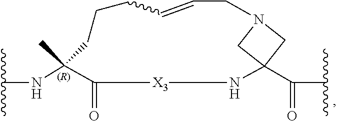

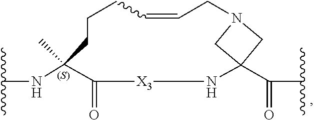

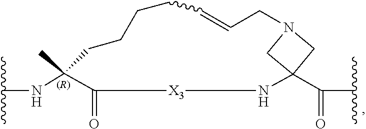

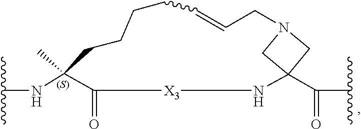

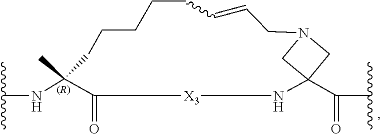

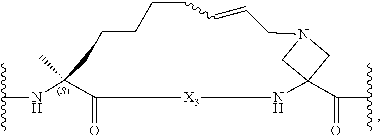

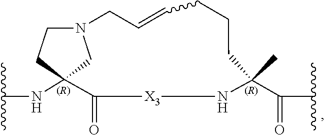

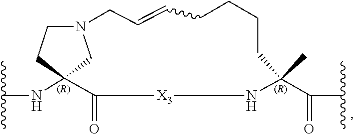

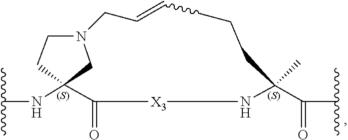

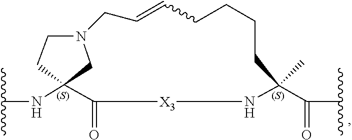

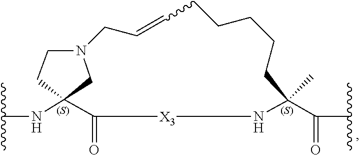

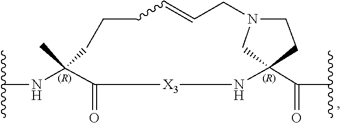

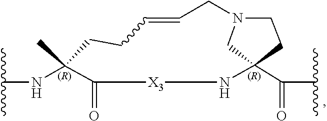

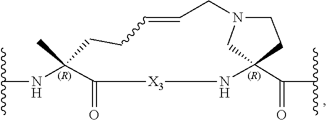

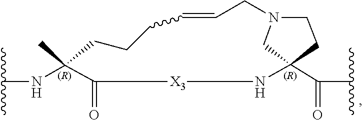

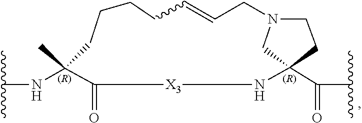

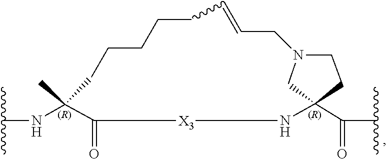

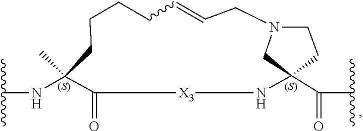

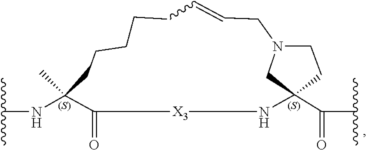

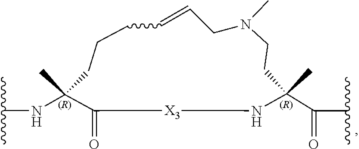

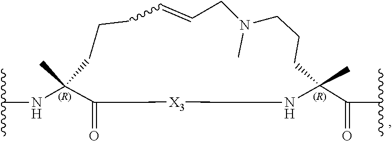

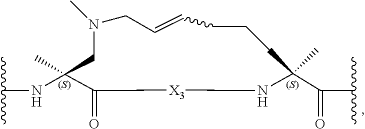

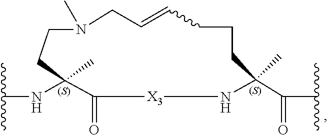

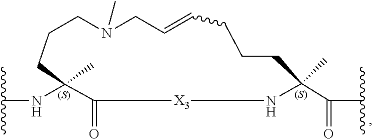

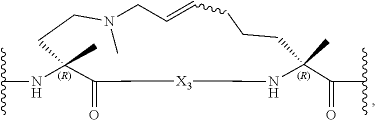

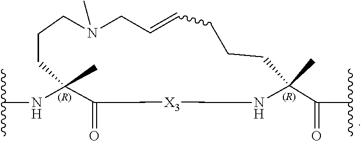

TABLE S-3
Exemplary staples.

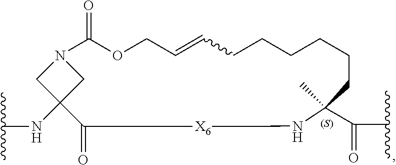

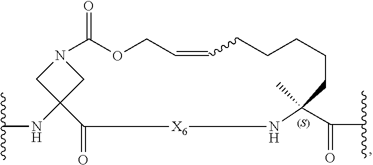

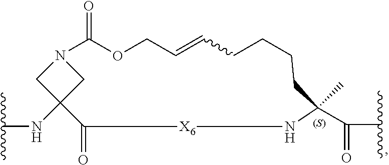

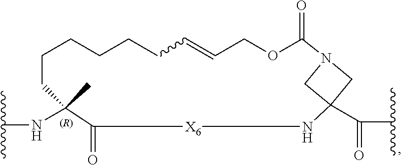

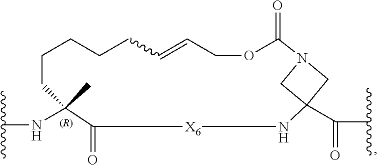

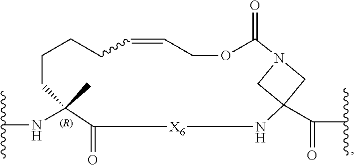

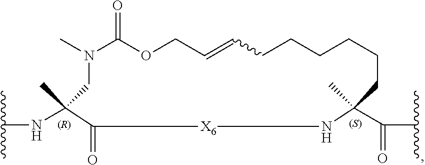

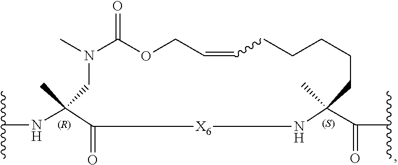

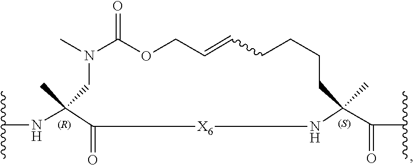

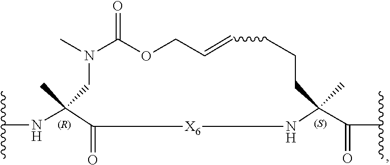

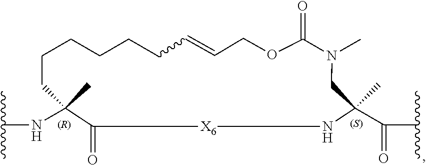

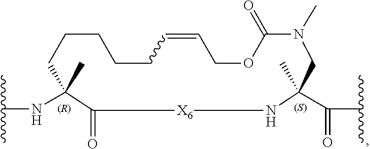

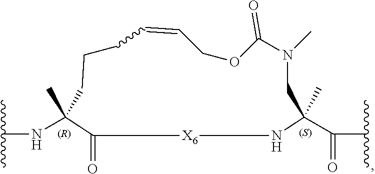

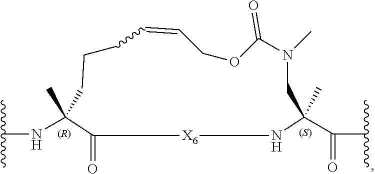

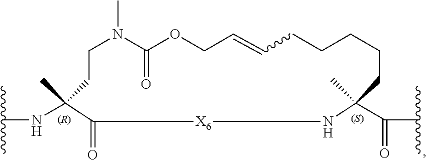

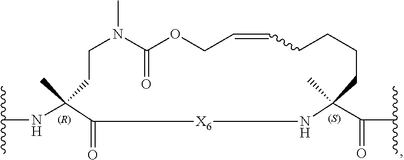

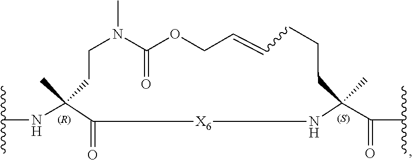

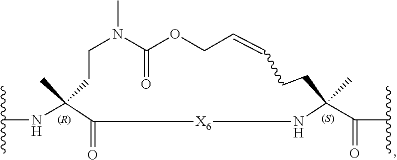

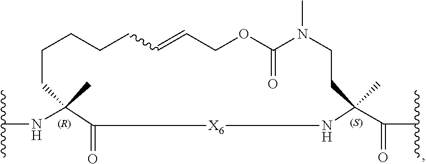

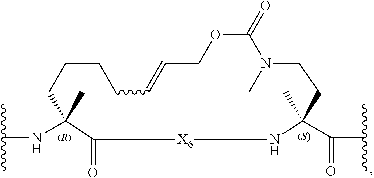

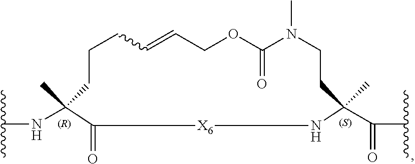

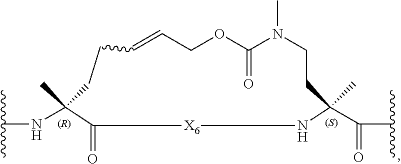

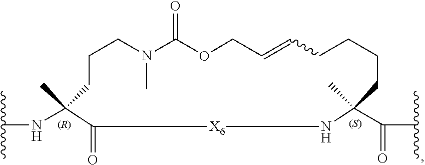

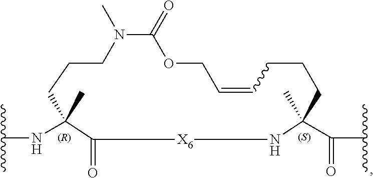

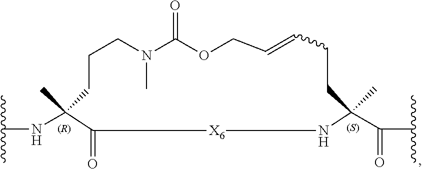

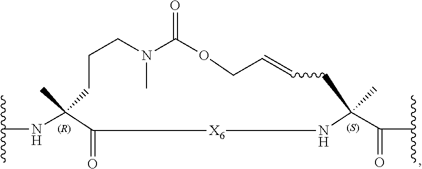

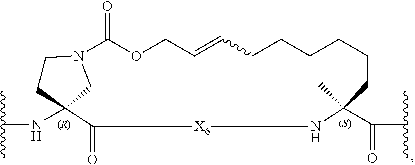

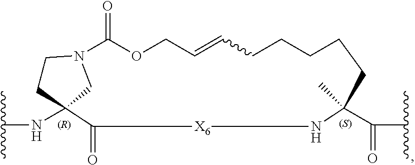

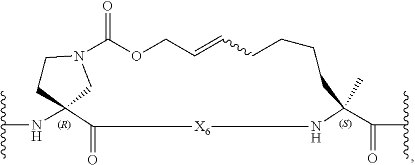

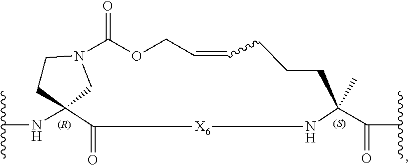

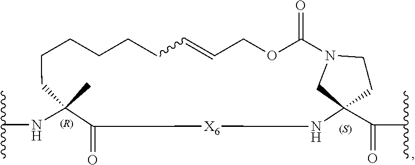

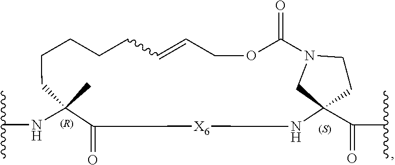

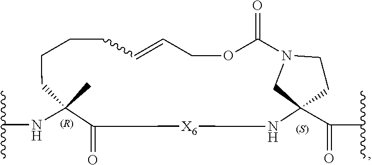

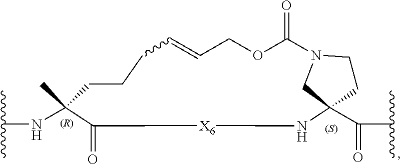

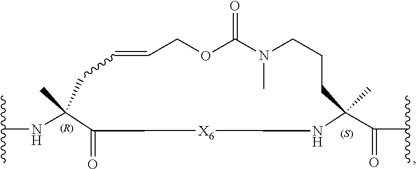

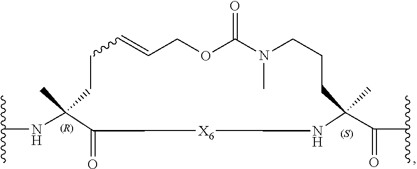

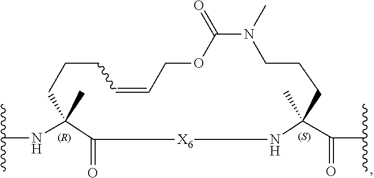

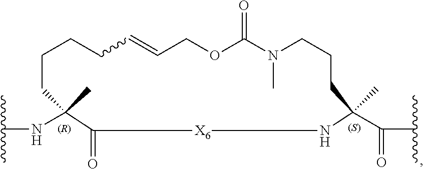

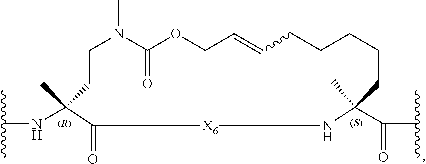

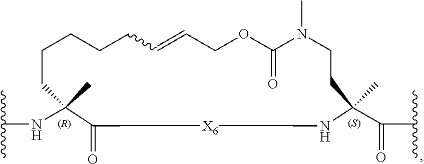

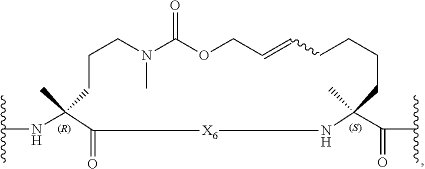

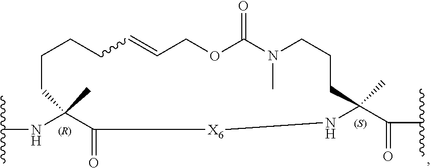

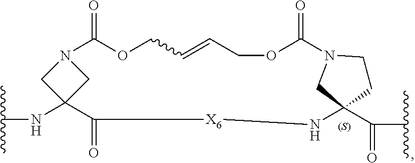

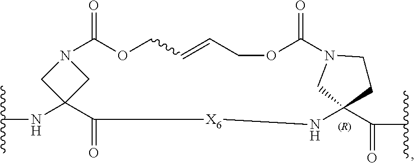

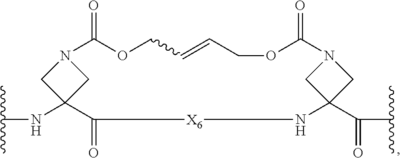

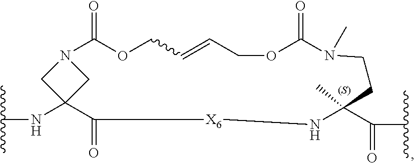

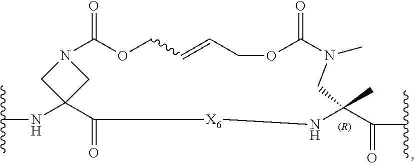

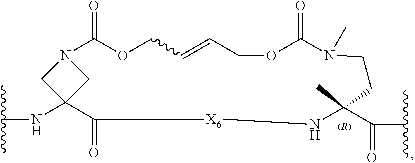

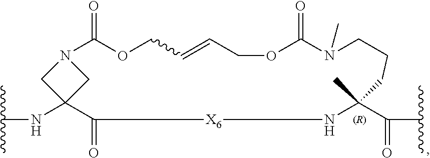

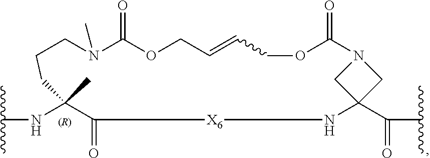

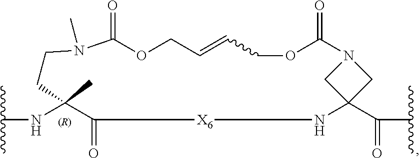

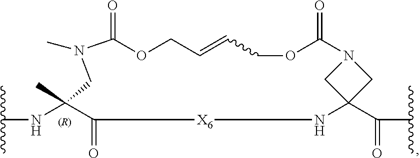

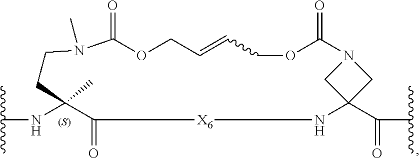

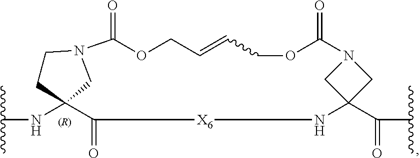

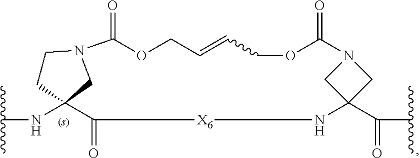

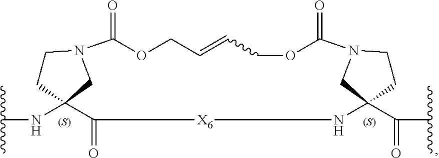

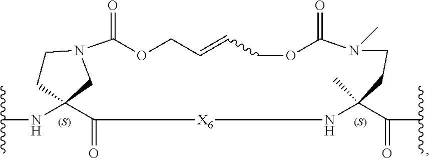

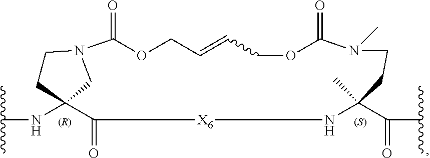

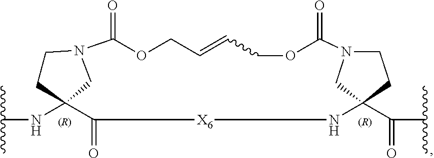

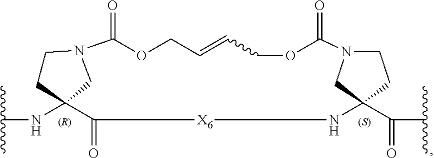

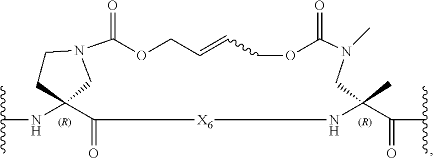

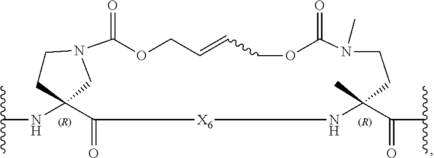

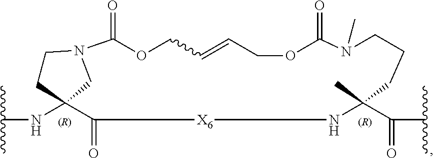

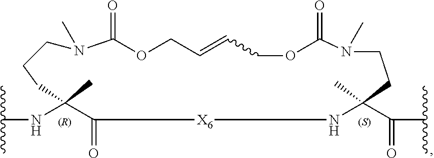

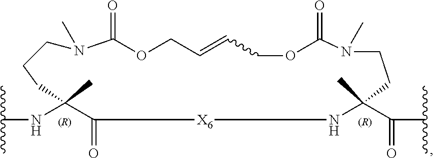

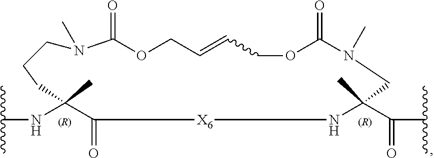

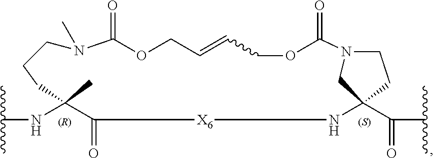

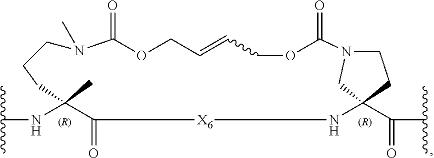

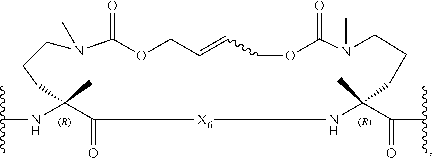

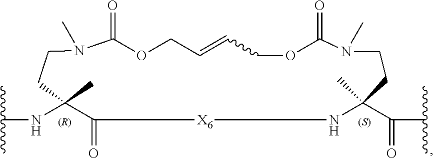

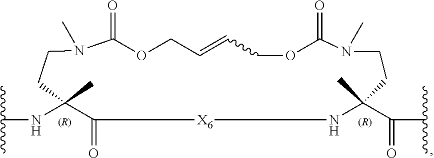

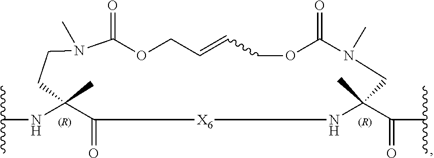

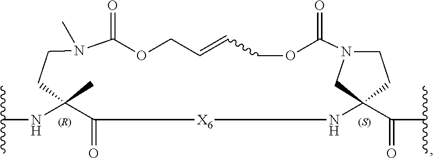

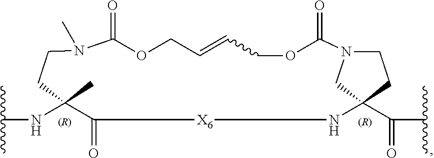

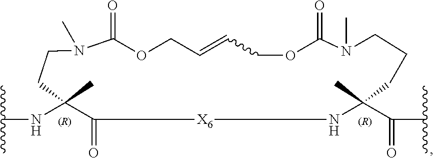

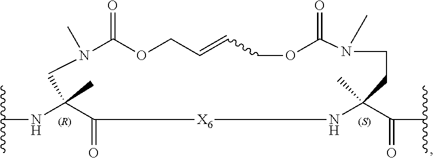

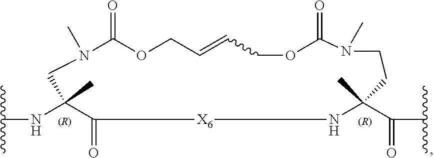

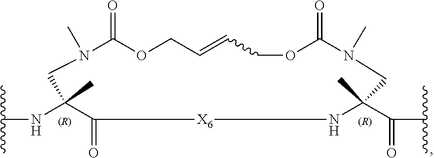

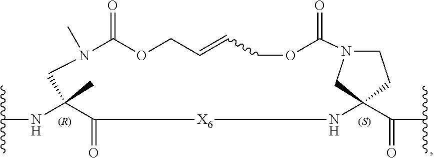

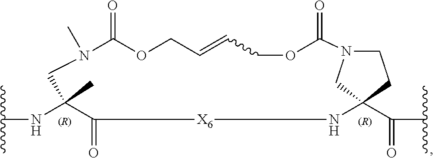

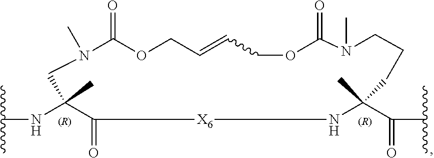

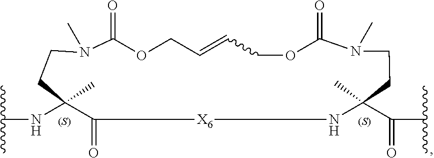

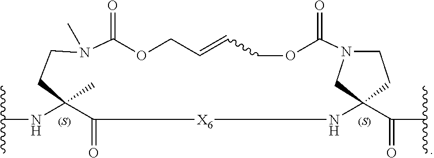

TABLE S-4
Exemplary staples.

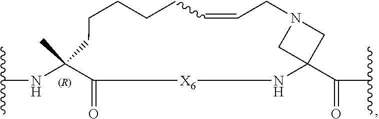

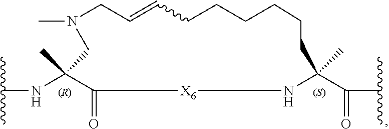

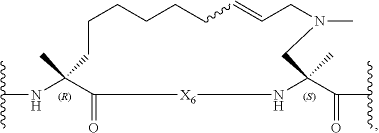

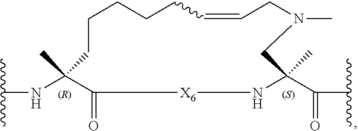

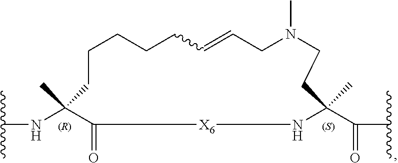

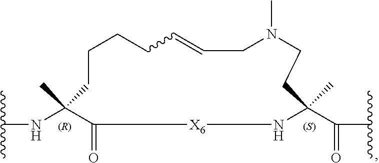

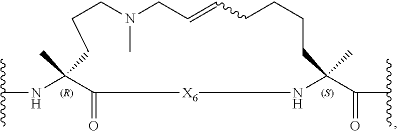

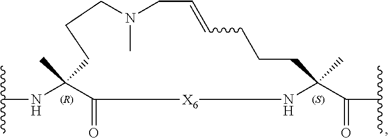

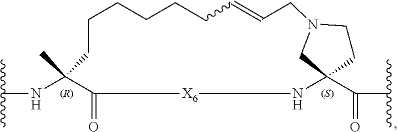

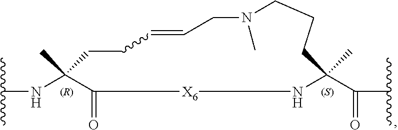

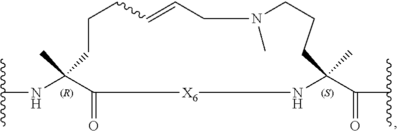

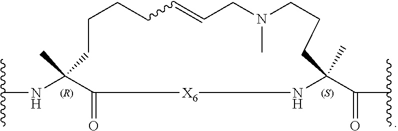

In some embodiments, a staple may be one of the following, connecting the amino acids at the indicated position:

TABLE S-5
Certain amino acids and staples.

| Amino Acid | Amino Acid 2 | |
|---|---|---|
| 1 | (i + 7 | staple |
| Monomer A | [symbol] | 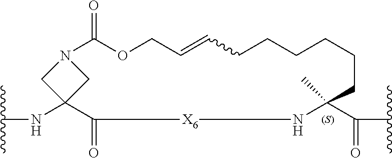 |
| Monomer A | S₇ | 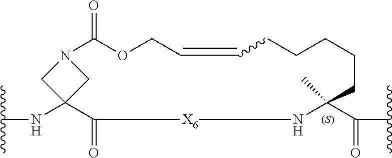 |
| Monomer A | S₆ | 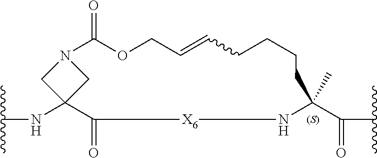 |
| R₈ | Monomer A | 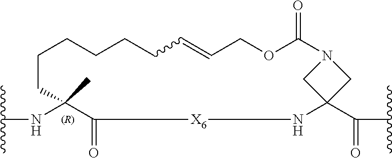 |
| R₇ | Monomer A | 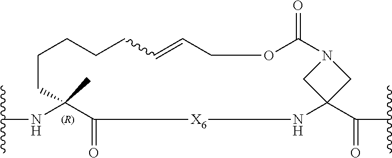 |
| R₆ | Sonomer A | 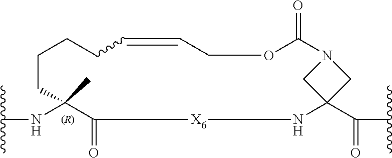 |
| Monomer E | [symbol] | 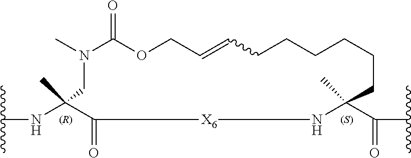 |
| Monomer E | S₇ | 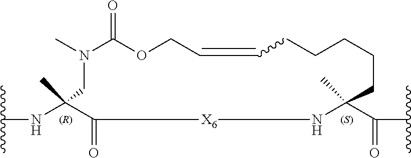 |
| Monomer E | S₆ | 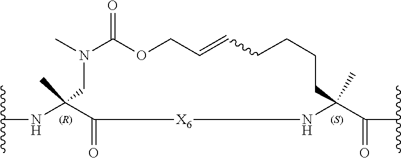 |
| Monomer E | S₅ | 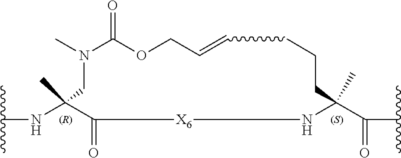 |
| R₈ | Monomer D | 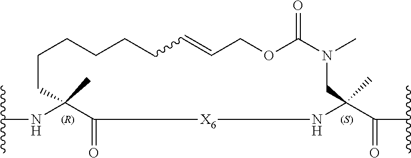 |
| R₇ | Monomer D | 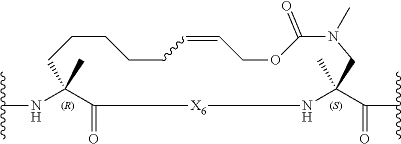 |
| R₆ | Monomer D | 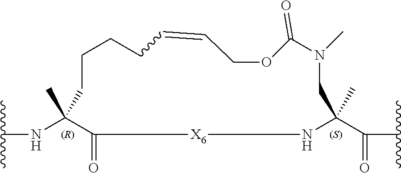 |
| R₅ | Monomer D | 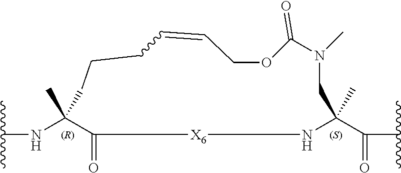 |
| Monomer G | S₇ | 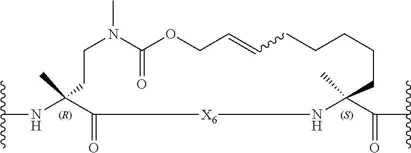 |
| Monomer G | S₆ | 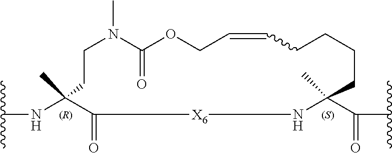 |
| Monomer G | S₅ | 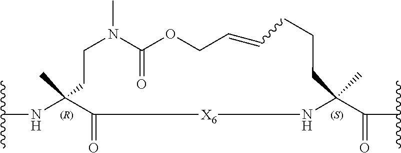 |
| Monomer G | S₄ | 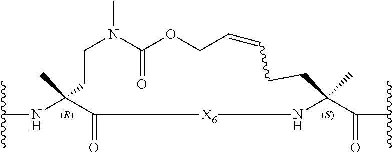 |
| R₇ | Monomer F | 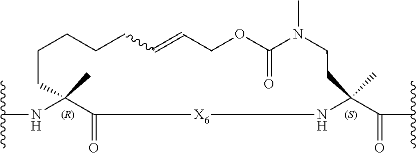 |
| R₆ | Monomer F | 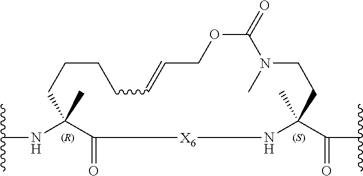 |
| R₅ | Monomer F | 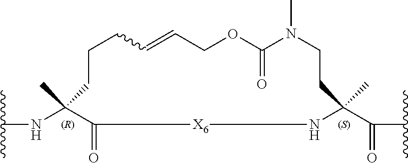 |
| [symbol] | Monomer F | 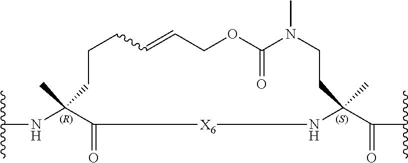 |
| R₄ | Monomer F | 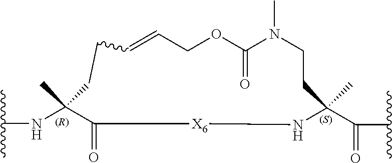 |
| Monomer I | S₆ | 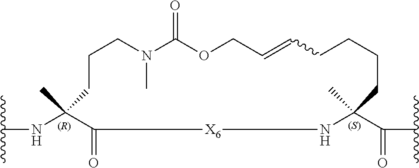 |
| Monomer I | S₅ | 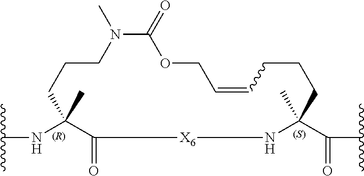 |
| Monomer I | S₄ | 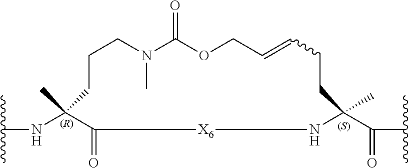 |
| Monomer I | S₃ | 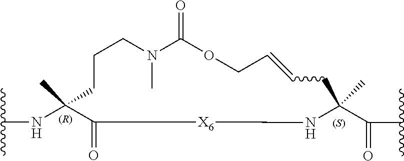 |
| Monomer C | [symbol] | 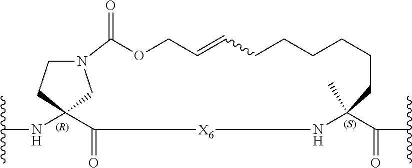 |
| Monomer C | S₇ | 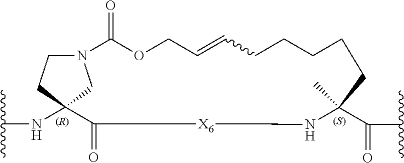 |
| Monomer C | S₆ | 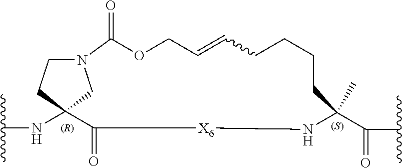 |
| Monomer C | S₅ | 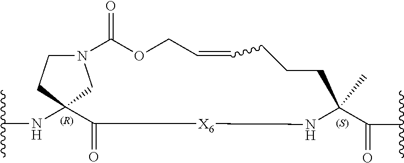 |
| R₈ | Monomer B | 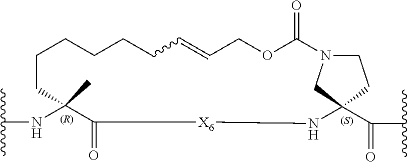 |
| R₇ | Monomer B | 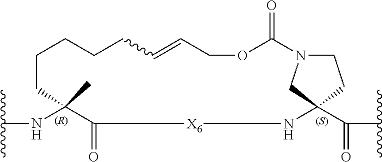 |
| R₆ | Monomer B | 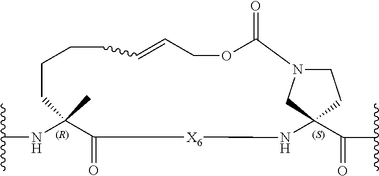 |
| R₅ | Monomer B | 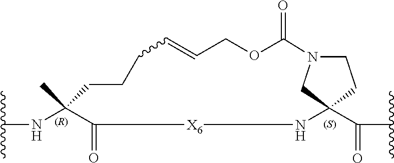 |
| R₃ | Monomer H | 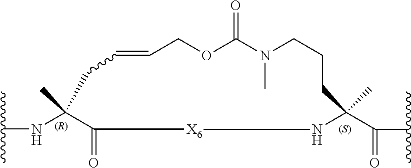 |
| R₄ | Monomer H | 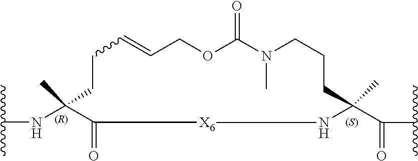 |
| R₅ | Monomer H | 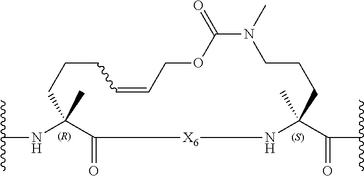 |
| R₆ | Monomer H | 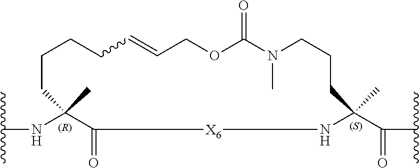 |
| Monomer G | S₇ | 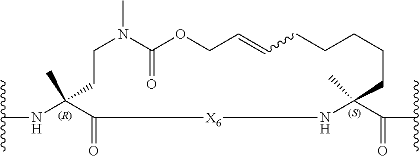 |
| R₇ | Monomer F | 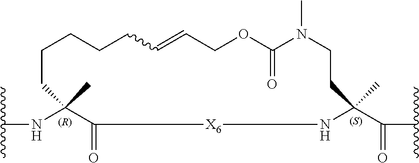 |
| Monomer I | S₆ | 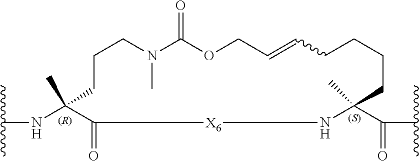 |
| R₆ | Monomer H | 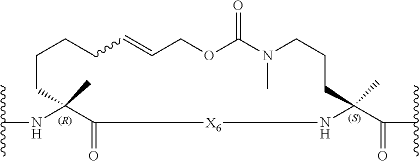 |
| Monomer A | Monomer B | 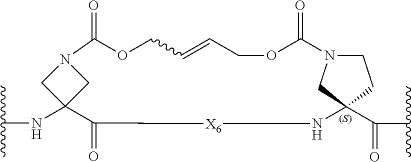 |
| Monomer A | Monomer C | 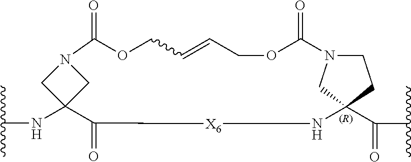 |
| Monomer A | Monomer A | 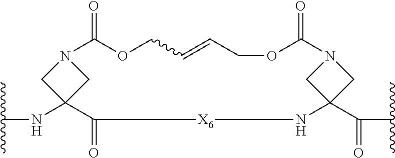 |
| Monomer A | Monomer F | 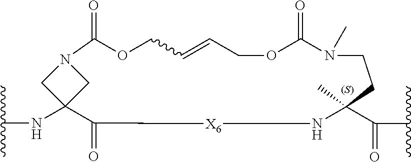 |
| Monomer A | Monomer E | 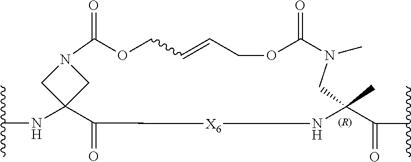 |
| Monomer A | Monomer G | 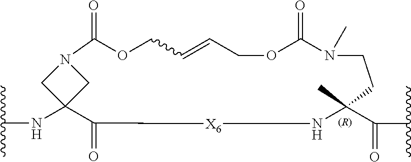 |
| Monomer A | Monomer I | 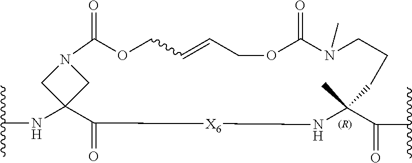 |
| Monomer I | Monomer A | 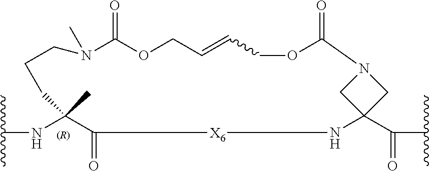 |
| Monomer G | Monomer A | 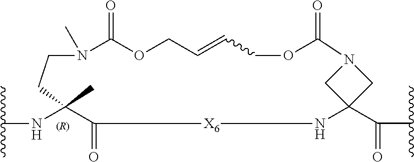 |
| Monomer E | Monomer A | 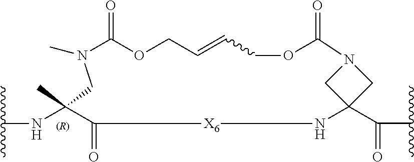 |
| Monomer F | Monomer A | 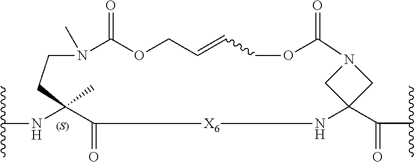 |
| Monomer C | Monomer A | 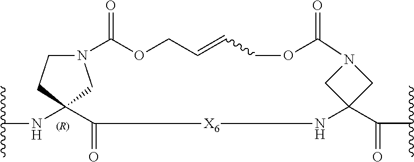 |
| Monomer B | Monomer A | 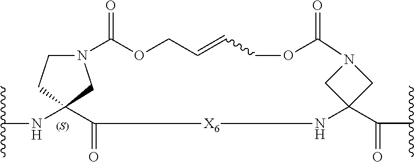 |
| Monomer B | Monomer B | 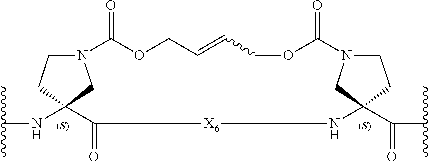 |
| Monomer B | Monomer F | 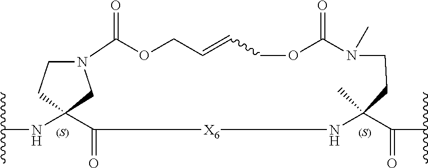 |
| Monomer C | Monomer F | 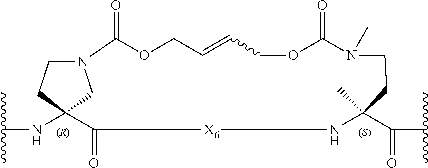 |
| Monomer C | Monomer C | 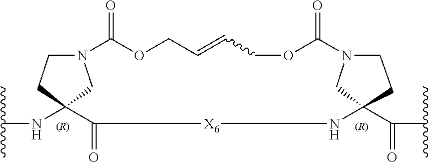 |
| Monomer C | Monomer B | 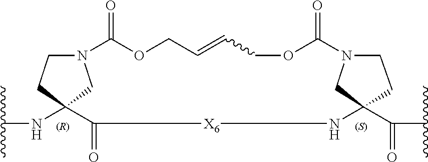 |
| Monomer C | Monomer E | 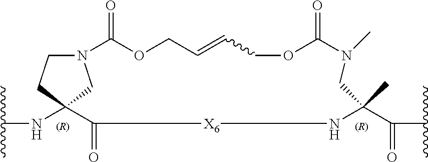 |
| Monomer C | Monomer G | 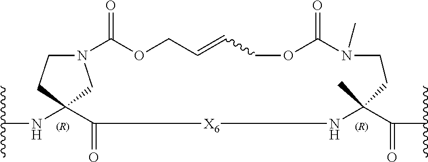 |
| Monomer C | Monomer I | 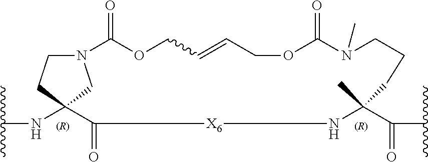 |
| Monomer I | Monomer F | 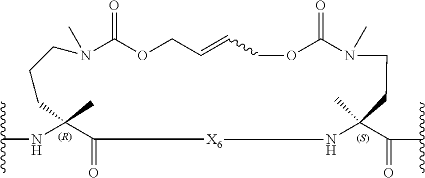 |
| Monomer I | Monomer G | 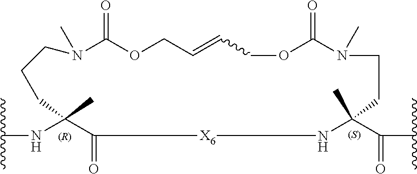 |
| Monomer I | Monomer E | 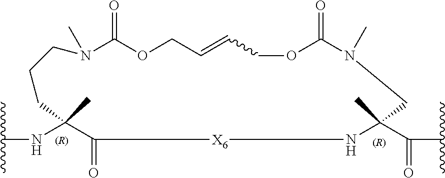 |
| Monomer I | Monomer B | 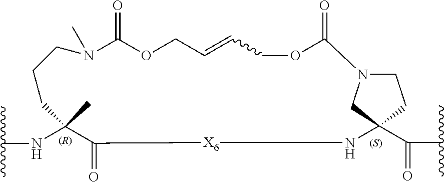 |
| Monomer I | Monomer C | 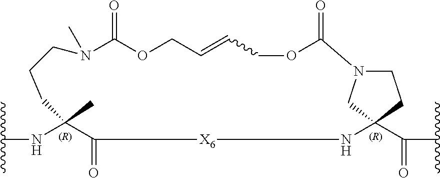 |
| Monomer I | Monomer I | 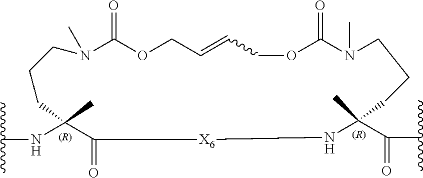 |
| Monomer G | Monomer F | 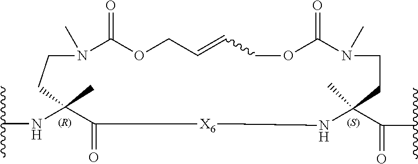 |
| Monomer G | Monomer G | 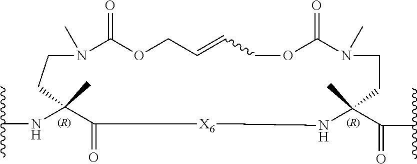 |
| Monomer G | Monomer E | 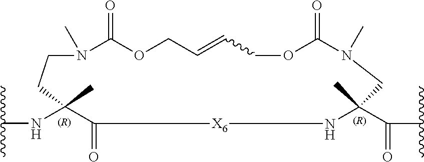 |
| Monomer G | Monomer B | 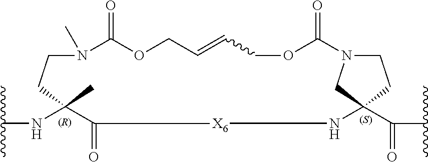 |
| Monomer G | Monomer C | 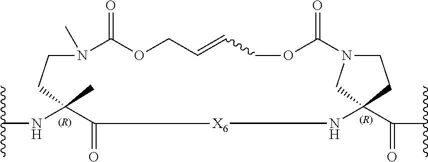 |
| Monomer G | Monomer I | 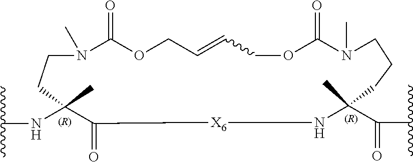 |
| Monomer E | Monomer F | 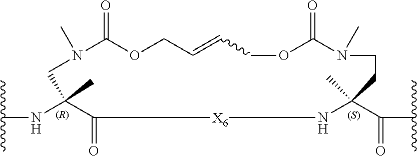 |
| Monomer E | Monomer G | 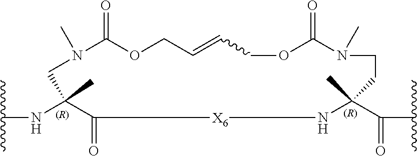 |
| Monomer E | Monomer E | 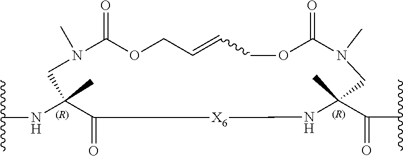 |
| Monomer E | Monomer B | 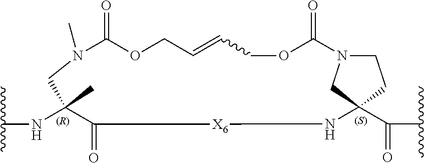 |
| Monomer E | Monomer C | 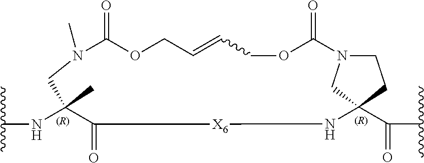 |
| Monomer E | Monomer I | 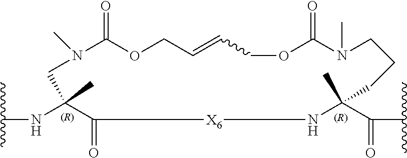 |
| Monomer F | Monomer F | 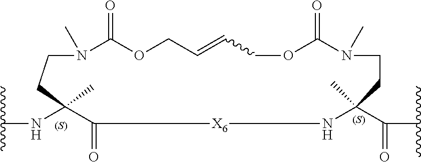 |
| Monomer F | Monomer B | 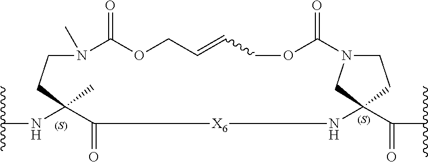 |
| R₇ | Monomer A | 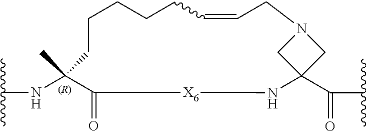 |
| Monomer E | [symbol] | 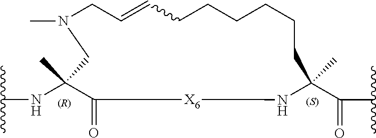 |
| R₈ | Monomer D | 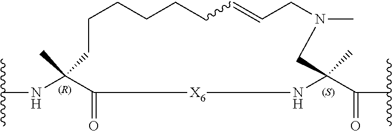 |
| R₇ | Monomer D | 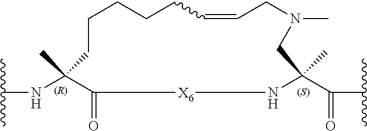 |
| R₇ | Monomer F | 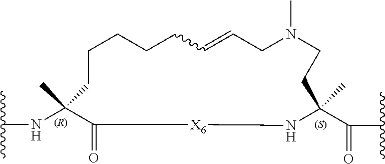 |
| R₆ | Monomer F | 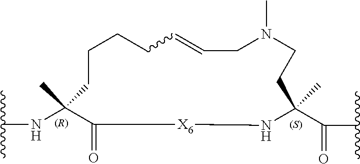 |
| Monomer I | [symbol] | 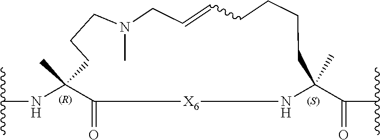 |
| Monomer I | [symbol] | 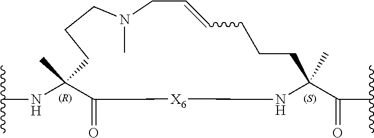 |
| R₈ | Monomer B | 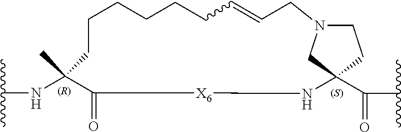 |
| R₄ | Monomer H | 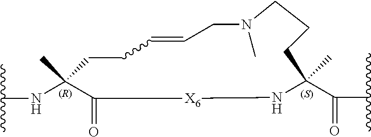 |
| R₅ | Monomer H | 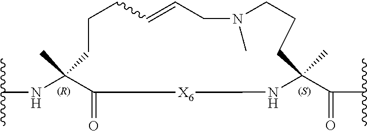 |
| R₆ | Monomer H | 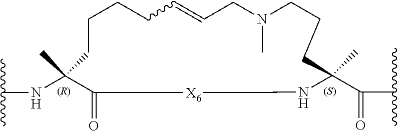 |

[symbol]

indicates data missing or illegible when filed

In some embodiments, it is C to N. In some embodiments, a double bond is E. In some embodiments, a double bond is Z. In some embodiments, a staple is a (i, i+3) staple. In some embodiments, a staple is a (i, i+4) staple. In some embodiments, a staple is a (i, i+7) staple. In some embodiments, each double is independently E or Z when a structure comprises more than one double bond. In some embodiments, each staple is independently a (i, i+3) or a (i, i+4) staple or a (i, i+7) staple. In some embodiments, each staple is independently a (i, i+4) staple or a (i, i+7) staple in a structure comprising two staples. In some embodiments, one staple is a (i, i+4) staple and the other is a (i, i+7) staple. In some embodiments, a PL3 residue is bonded to a (i, i+3) staple. In some embodiments, a PL3 residue is bonded to a (i, i+4) staple. In some embodiments, staples (e.g., those in Table 6) are formed by metathesis of double bonds in side chains of amino acid residues, e.g., RdN and S7, R8 and PyrS, R5 and SeN, R6 and SeN, ReN and S5, ReN and S6, R7 and PyrS, Az and S7, R8 and SgN, Az and S8, R4 and SeN, R5 and SdN, R7 and Az, R8 and Az, RdN and S4, RgN and S8, RgN and S7, R8 and S5, PL3 and B5 and the same B5 and S8, PL3 and B5 and the same B5 and SeN, PL3 and B5 and the same B5 and SdN, PL3 and B5 and the same B5 and S7, PL3 and B5 and the same B5 and PyrS2, PL3 and B5 and the same B5 and PyrS3, R5 and PyrS2, PL3 and B5 and the same B5 and PyrS1, PL3 and B5 and the same B5 and S10, PL3 and B5 and the same B5 and PyrR2, PL3 and B5 and the same B5 and PyrS, PL3 and B5 and the same B5 and Az, PL3 and B5 and the same B5 and SeNc5, HypEs5 and B5 and the same B5 and PyrS2, HypEs4 and B5 and the same B5 and PyrS2, ProSAm3 and B5 and the same B5 and PyrS2, ProAm5 and B5 and the same B5 and PyrS2, ProAm6 and B5 and the same B5 and PyrS2, BzAm3Oallyl and B5 and the same B5 and PyrS2, HypBzEs3OAllyl and B5 and the same B5 and PyrS2, ProBzAm3OAllyl and B5 and the same B5 and PyrS2, PAc3OAllyl and B5 and the same B5 and PyrS2, ProPAc3OAllyl and B5 and the same B5 and PyrS2, HypPAc3OAllyl and B5 and the same B5 and PyrS2, Bn3OAllyl and B5 and the same B5 and PyrS2, R3 and B5 and the same B5 and PyrS2, R5 and B5 and the same B5 and PyrS2, BzAm2Allyl-MePro and B5 and the same B5 and PyrS2, PL3 and B5 and the same B5 and SPip1, PL3 and B5 and the same B5 and SPip2, PL3 and B5 and the same B5 and SPip3, PL3 and B5 and the same B5 and Az2, PL3 and B5 and the same B5 and Az3, PL3 and S5, R5 and S5, PL3 and B4 and the same B4 and PyrS1, PL3 and B4 and the same B4 and PyrS2, PL3 and B4 and the same B4 and PyrS3, PL3 and S6, PL3 and S4, PL3 and S3, R6 and PyrS2, R4 and PyrS2, R3 and PyrS2, PL3 and B3 and the same B3 and PyrS2, PL3 and B3 and the same B3 and PyrS3, PL3 and B3 and the same B3 and PyrS4, PL3 and B6 and the same B6 and PyrS, PL3 and B6 and the same B6 and PyrS1, PL3 and B6 and the same B6 and PyrS2, 5hexenyl-MePro and B5 and the same B5 and PyrS2, 4pentenyl-MePro and B5 and the same B5 and PyrS2, 3butenyl-MePro and B5 and the same B5 and PyrS2.

TABLE S-6
Certain staples (including amino acid residues bonded to staples).

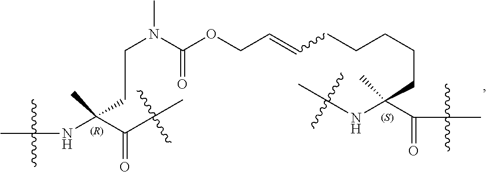

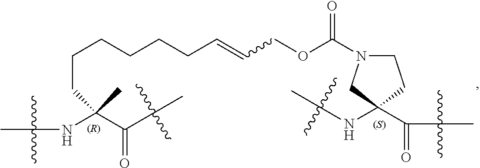

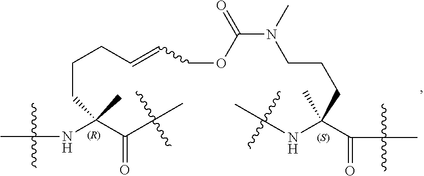

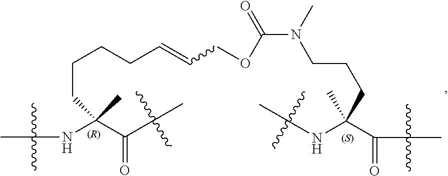

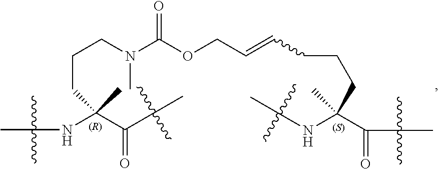

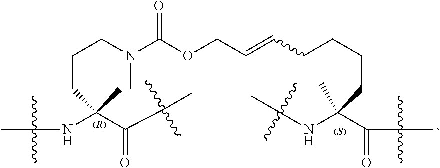

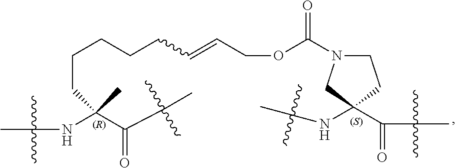

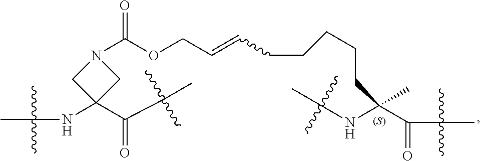

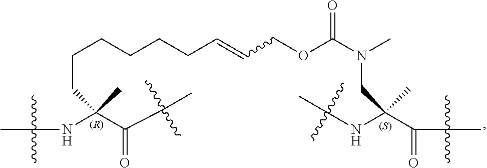

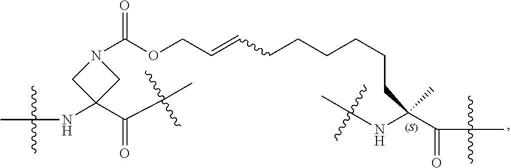

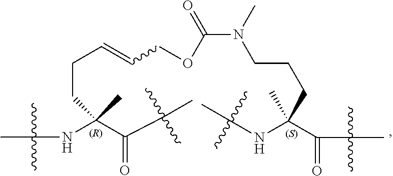

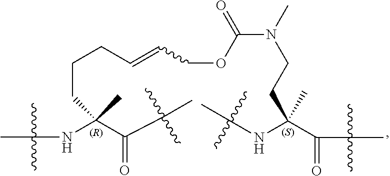

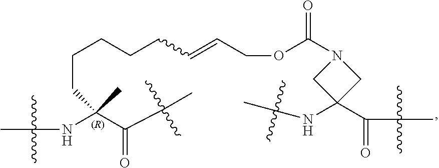

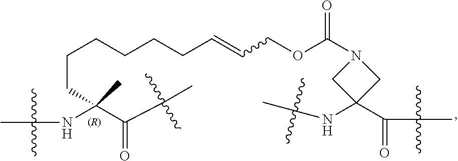

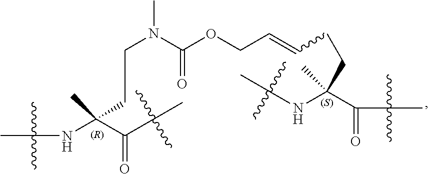

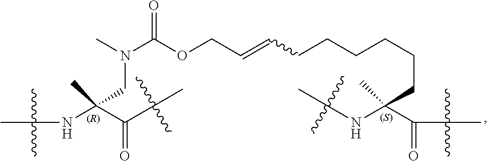

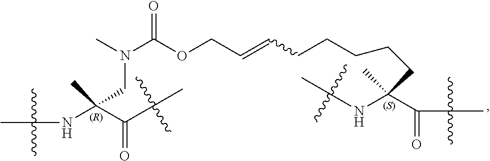

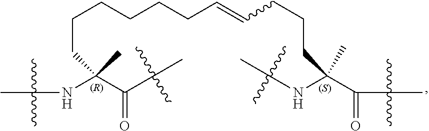

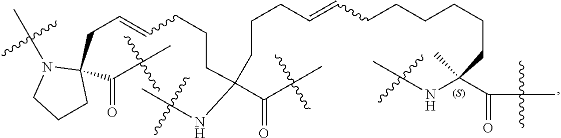

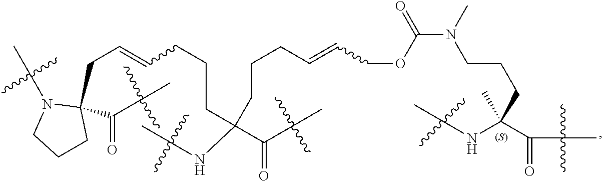

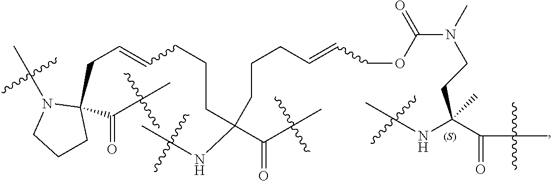

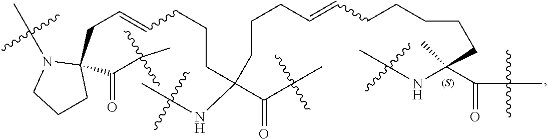

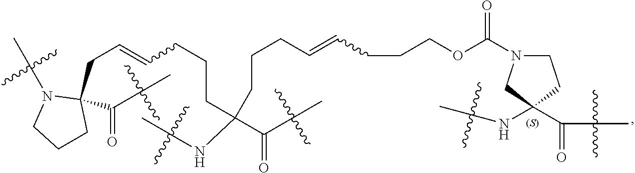

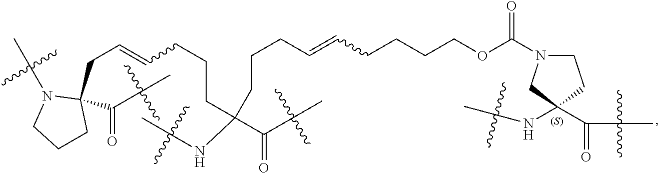

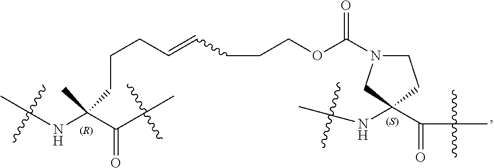

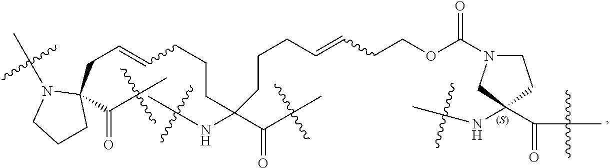

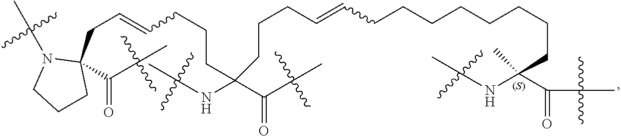

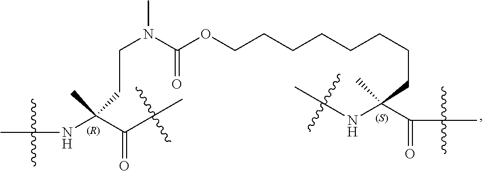

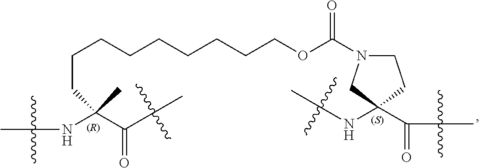

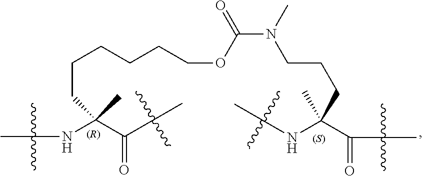

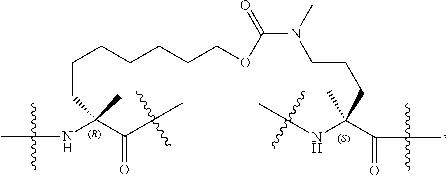

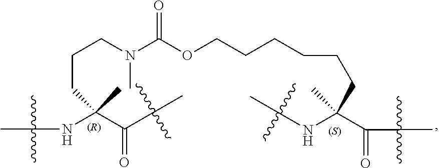

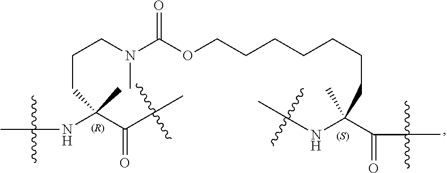

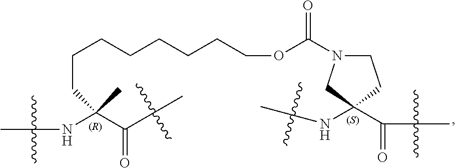

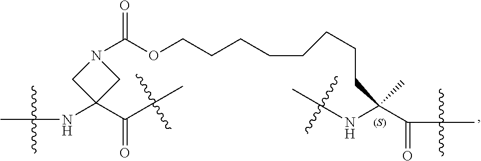

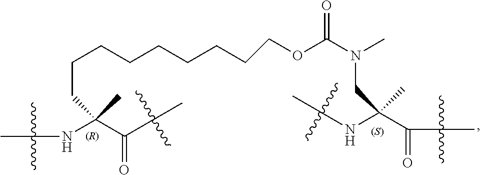

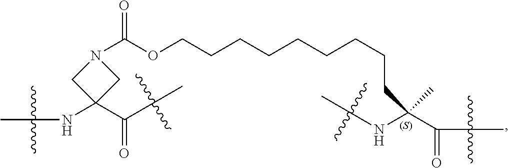

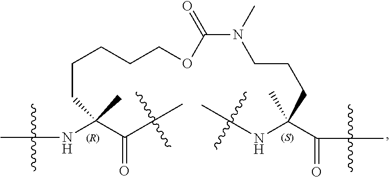

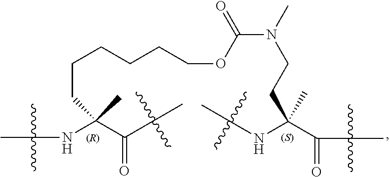

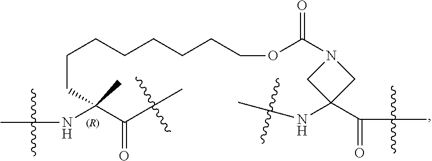

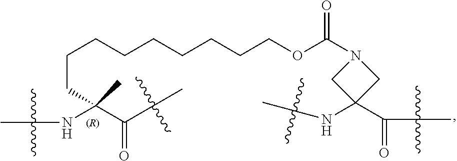

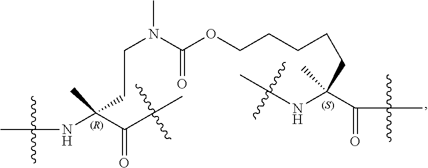

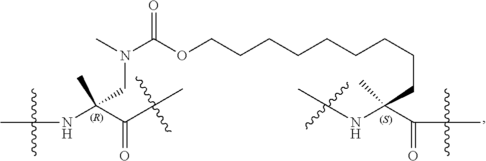

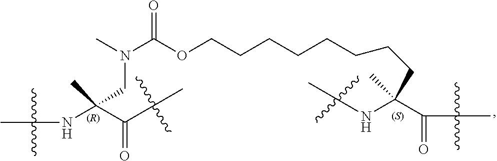

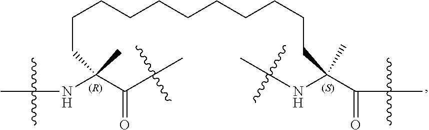

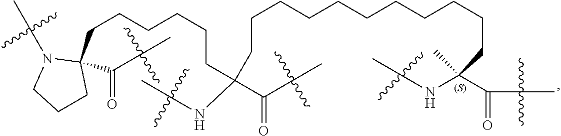

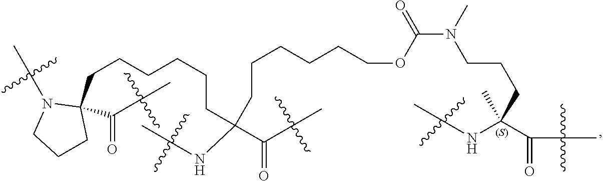

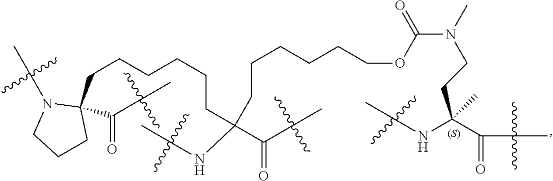

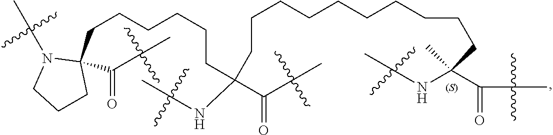

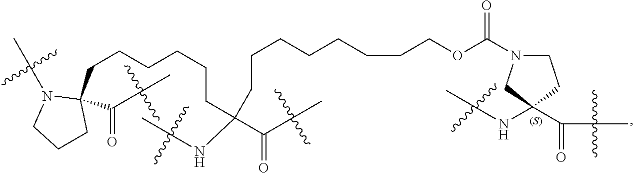

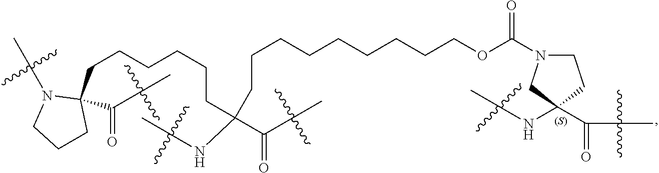

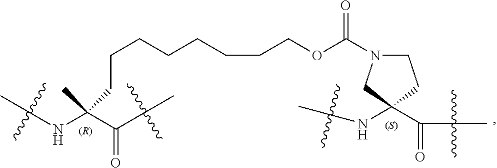

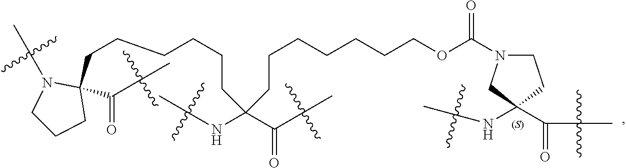

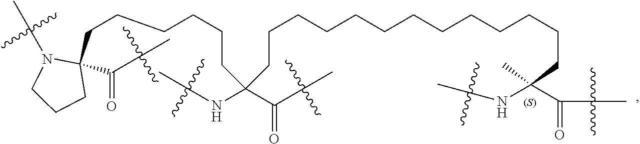

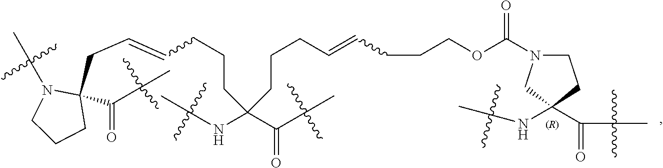

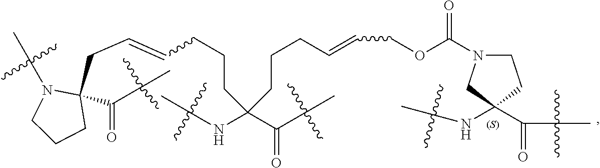

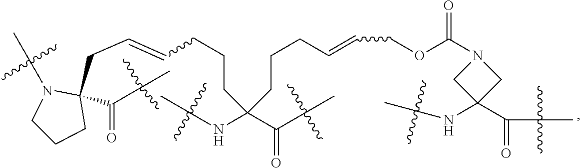 [SMILES: *NC(CCCC=CCOC(=O)N1CC(N*)(C(*)=O)C1)(CCCC=CC[C@@]1(C(*)=O)CCCN1*)C(*)=O]

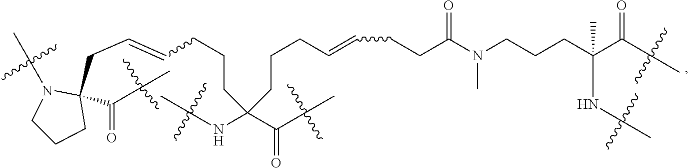 [SMILES: *NC(CCCC=CCCC(=O)N(C)CCC[C@](C)(N*)C(*)=O)(CCCC=CC[C@@]1(C(*)=O)CCCN1*)C(*)=O]

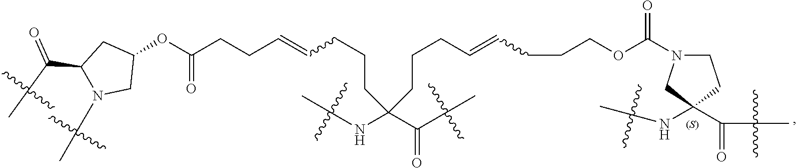

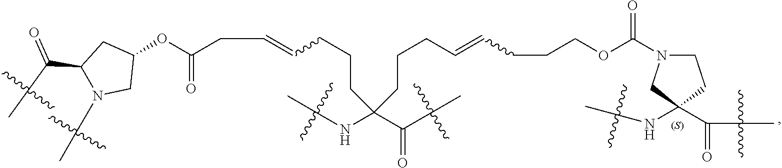

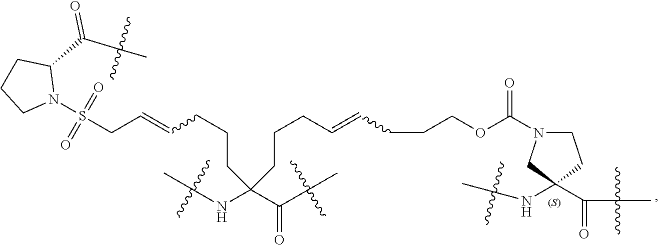

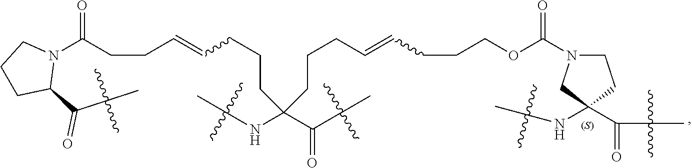

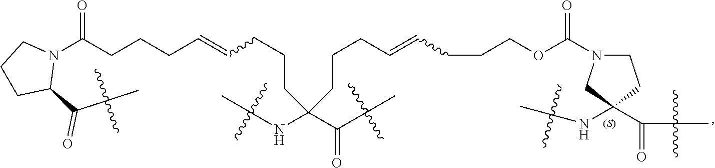

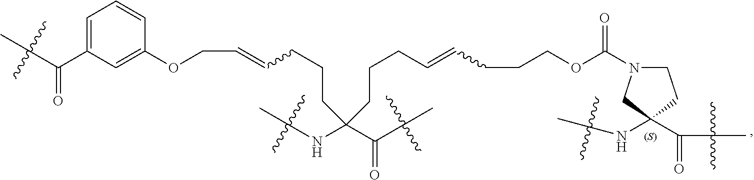

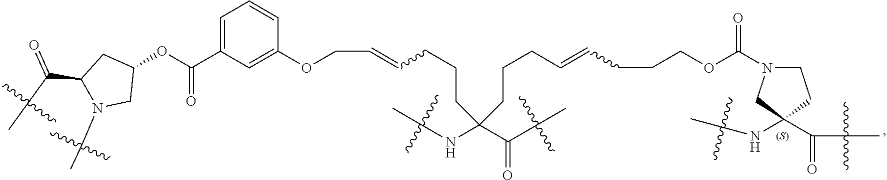

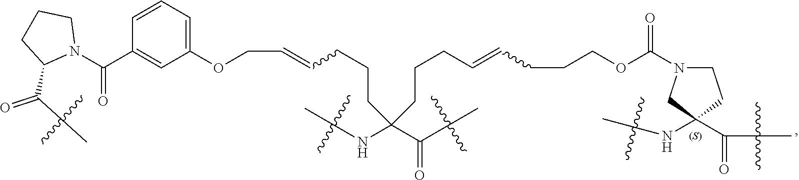

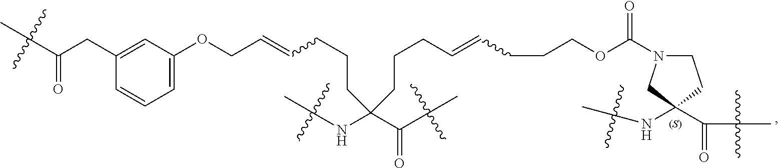

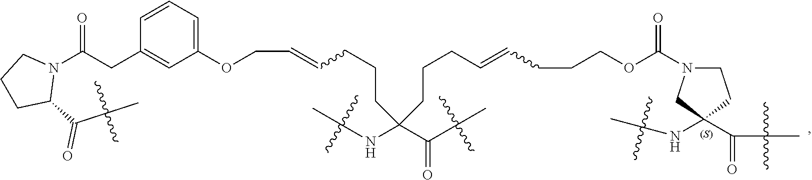

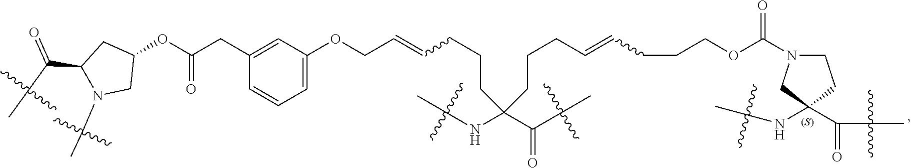

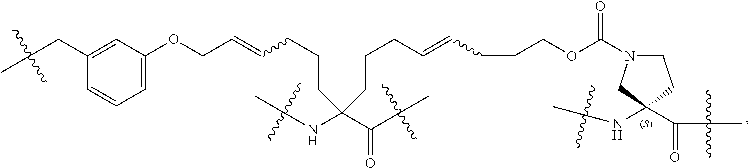

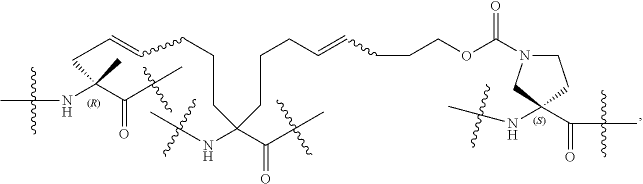

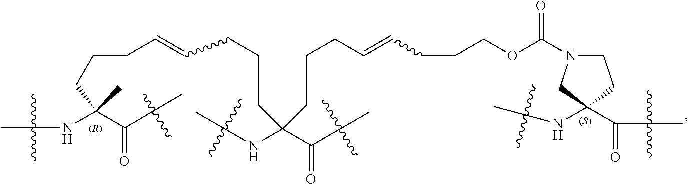

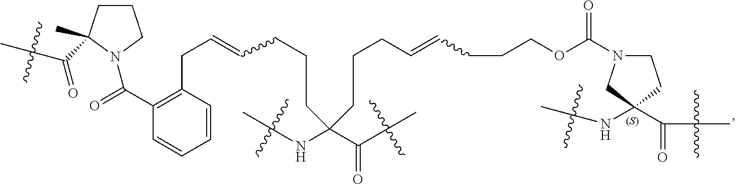

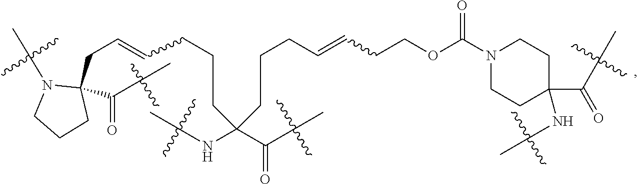 [SMILES: *NC(CCCC=CCCOC(=O)N1CCC(N*)(C(*)=O)CC1)(CCCC=CC[C@@]1(C(*)=O)CCCN1*)C(*)=O]

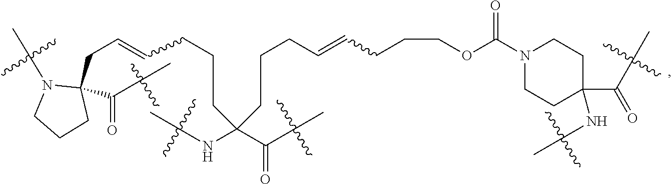 [SMILES: *NC(CCCC=CCCCOC(=O)N1CCC(N*)(C(*)=O)CC1)(CCCC=CC[C@@]1(C(*)=O)CCCN1*)C(*)=O]

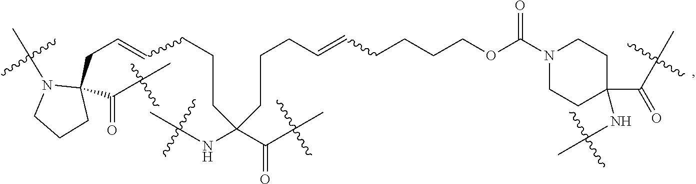 [SMILES: *NC(CCCC=CCCCCOC(=O)N1CCC(N*)(C(*)=O)CC1)(CCCC=CC[C@@]1(C(*)=O)CCCN1*)C(*)=O]

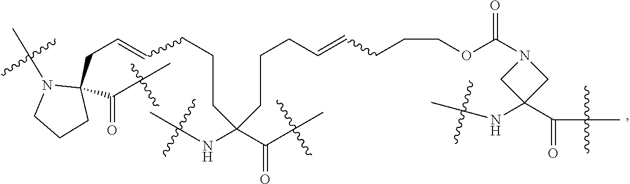 [SMILES: *NC(CCCC=CCCCOC(=O)N1CC(N*)(C(*)=O)C1)(CCCC=CC[C@@]1(C(*)=O)CCCN1*)C(*)=O]

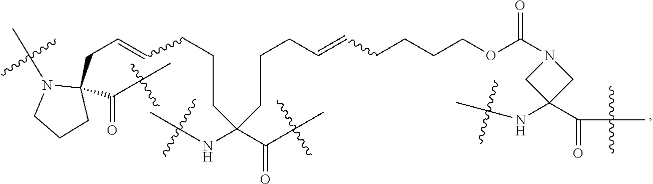 [SMILES: *NC(CCCC=CCCCCOC(=O)N1CC(N*)(C(*)=O)C1)(CCCC=CC[C@@]1(C(*)=O)CCCN1*)C(*)=O]

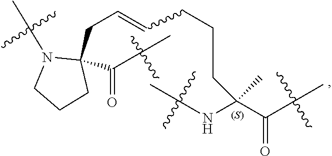

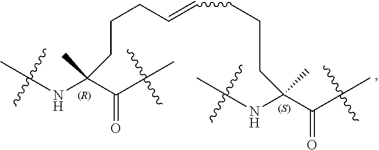

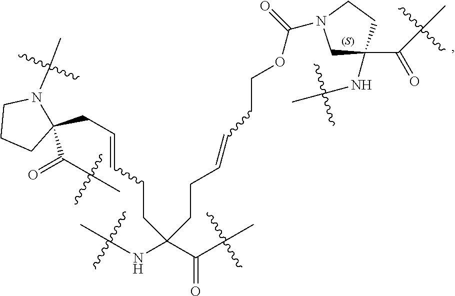

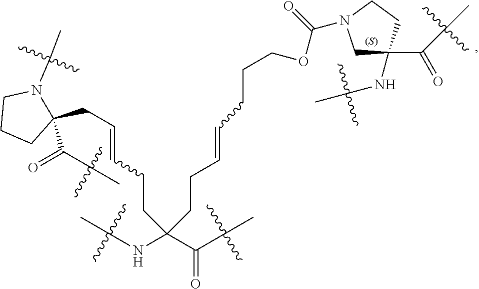

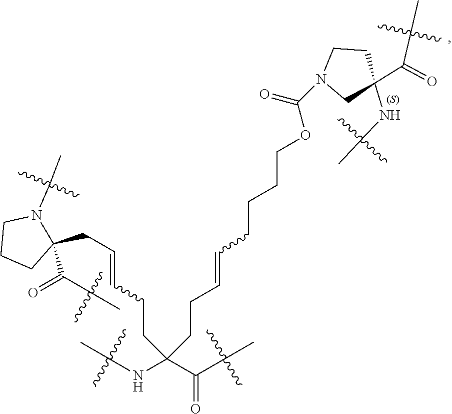

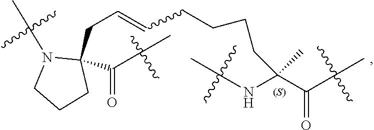

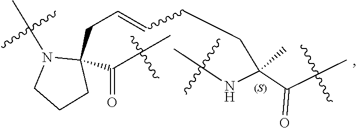

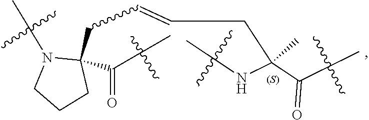

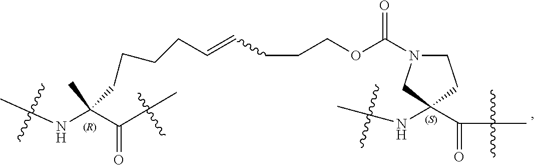

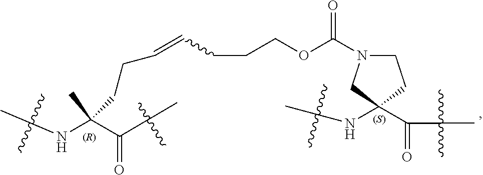

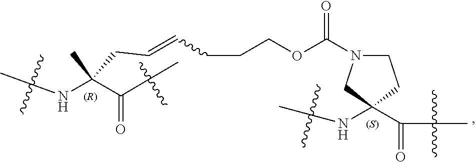

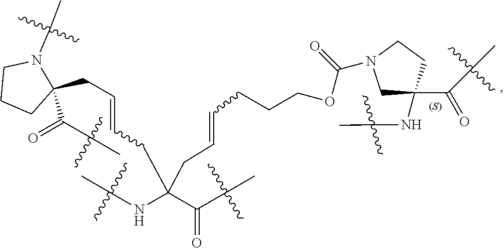

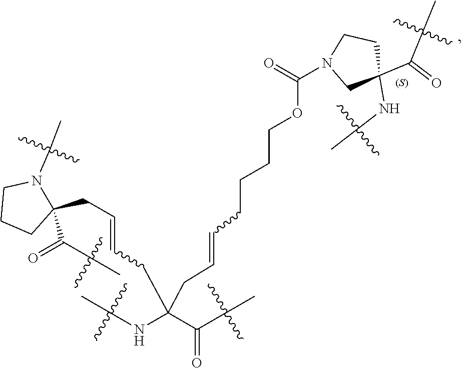

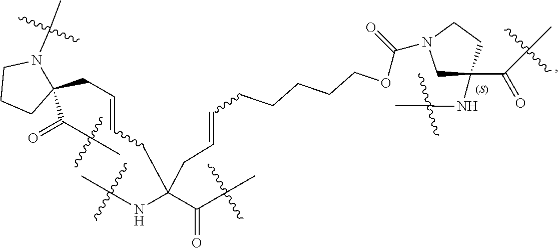

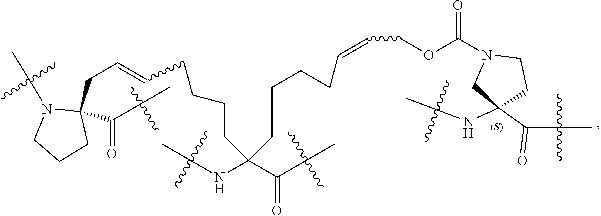

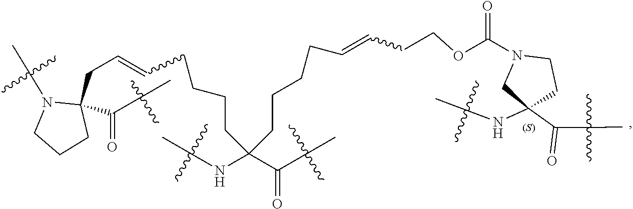

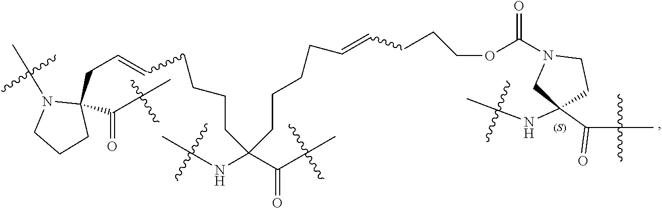

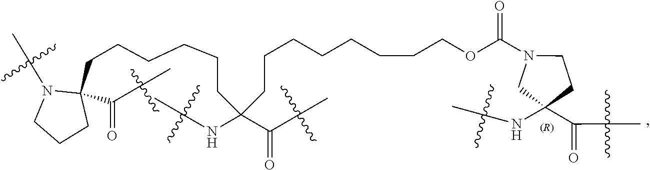

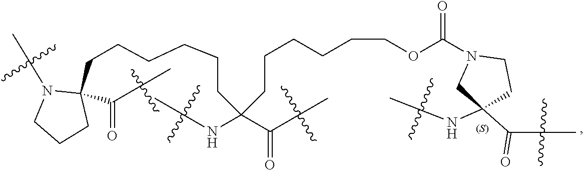

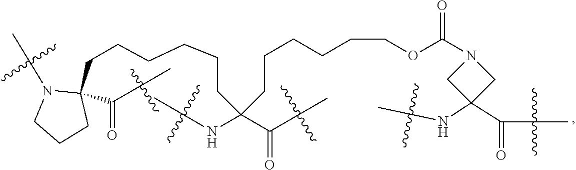 [SMILES: *NC(CCCCCCOC(=O)N1CC(N*)(C(*)=O)C1)(CCCCCC[C@@]1(C(*)=O)CCCN1*)C(*)=O]

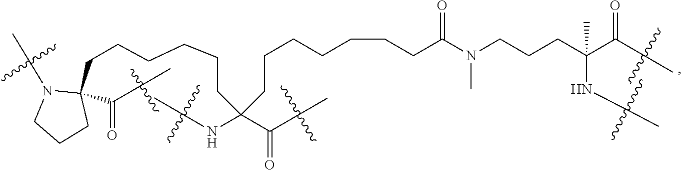 [SMILES: *NC(CCCCCCCC(=O)N(C)CCC[C@](C)(N*)C(*)=O)(CCCCCC[C@@]1(C(*)=O)CCCN1*)C(*)=O]

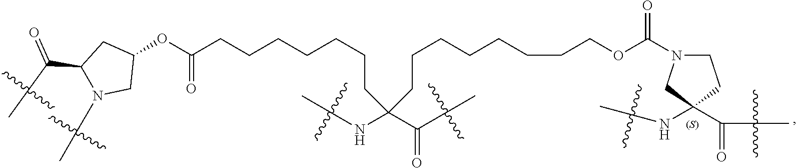

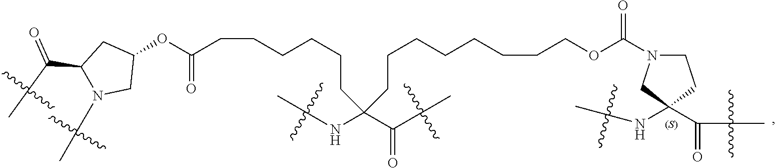

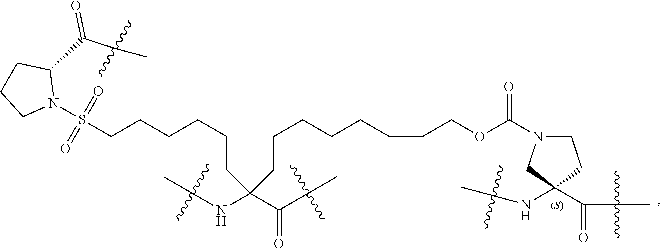

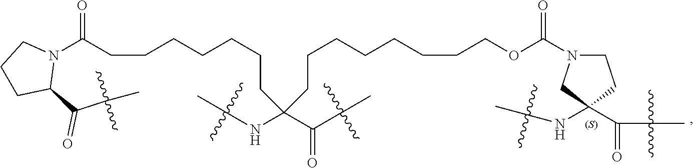

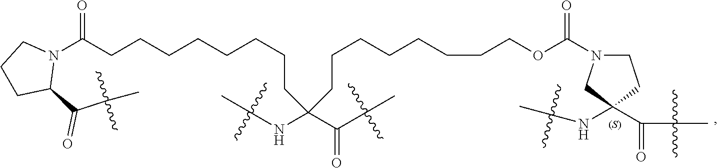

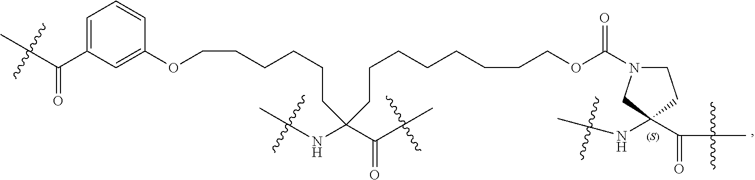

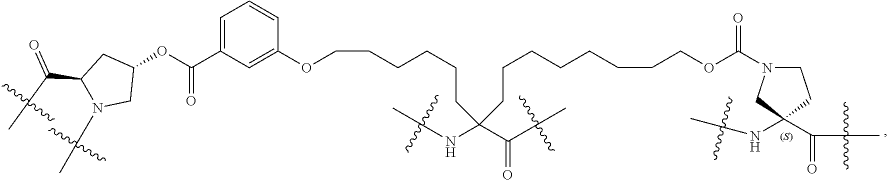

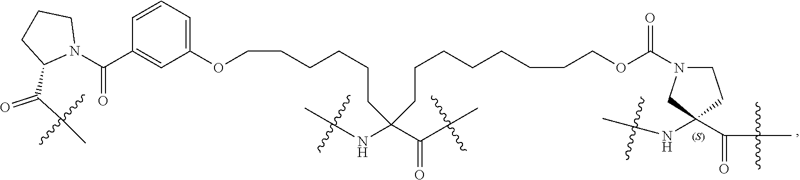

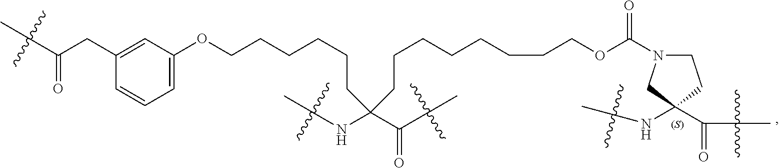

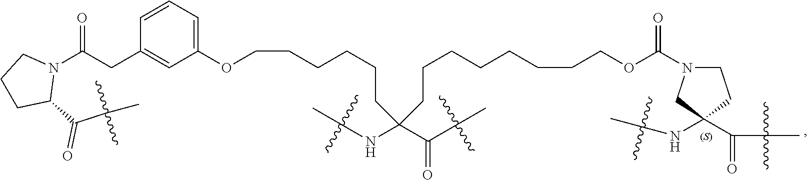

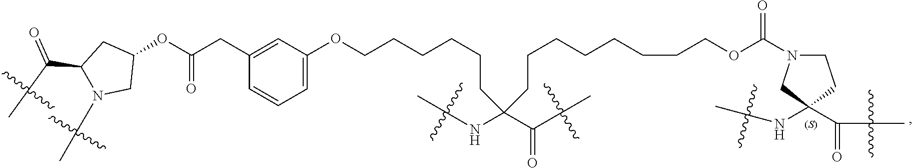

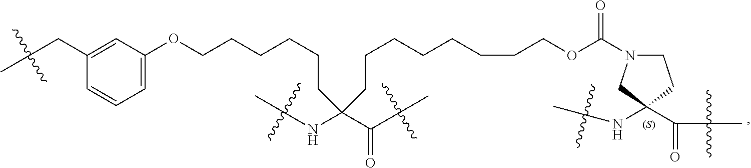

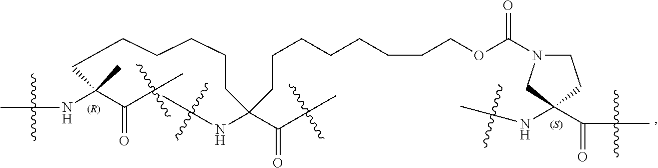

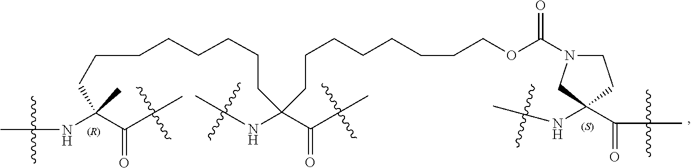

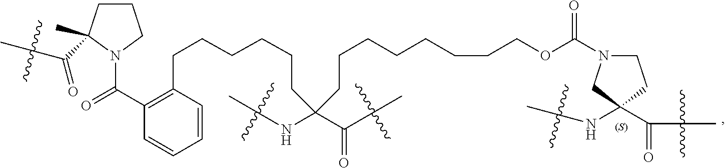

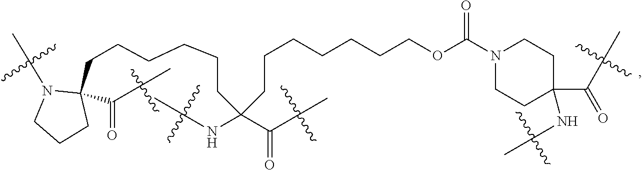 [SMILES: *NC(CCCCCCCOC(=O)N1CCC(N*)(C(*)=O)CC1)(CCCCCC[C@@]1(C(*)=O)CCCN1*)C(*)=O]

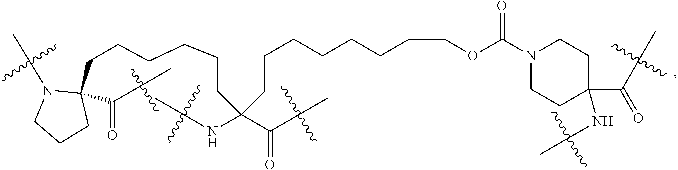 [SMILES: *NC(CCCCCCCCOC(=O)N1CCC(N*)(C(*)=O)CC1)(CCCCCC[C@@]1(C(*)=O)CCCN1*)C(*)=O]

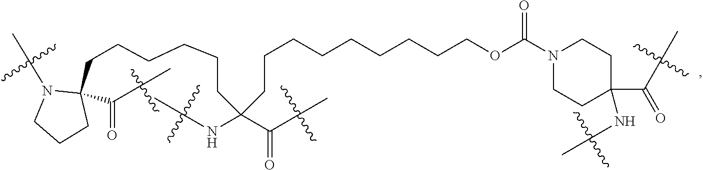 [SMILES: *NC(CCCCCCCCCOC(=O)N1CCC(N*)(C(*)=O)CC1)(CCCCCC[C@@]1(C(*)=O)CCCN1*)C(*)=O]

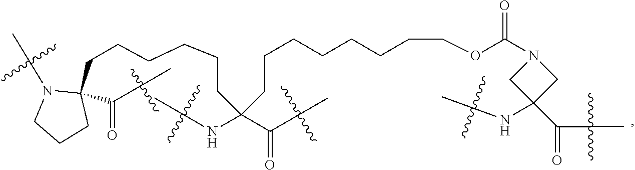 [SMILES: *NC(CCCCCCCCOC(=O)N1CC(N*)(C(*)=O)C1)(CCCCCC[C@@]1(C(*)=O)CCCN1*)C(*)=O]

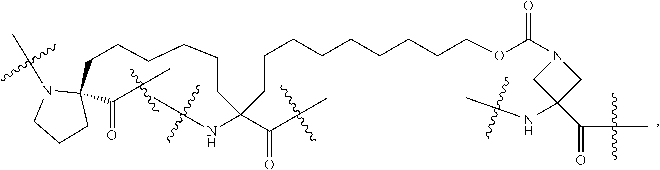 [SMILES: *NC(CCCCCCCCCOC(=O)N1CC(N*)(C(*)=O)C1)(CCCCCC[C@@]1(C(*)=O)CCCN1*)C(*)=O]

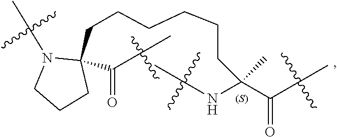

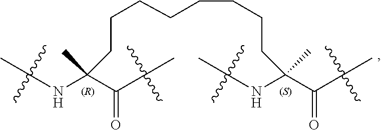

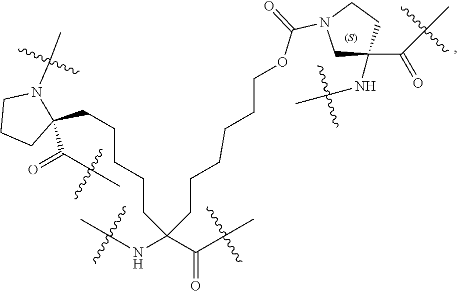

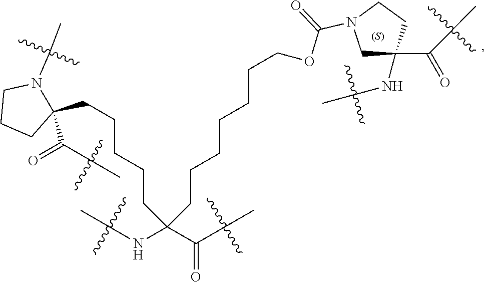

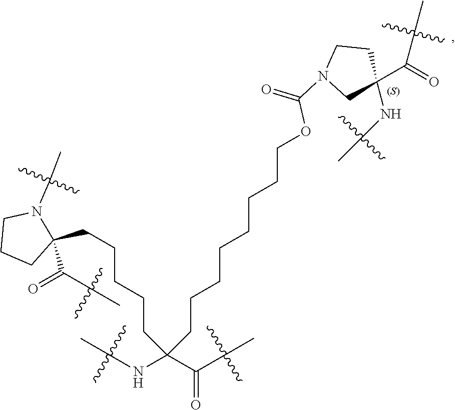

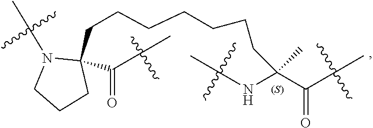

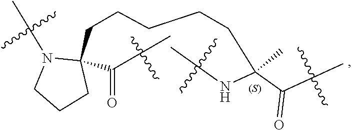

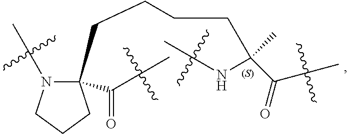

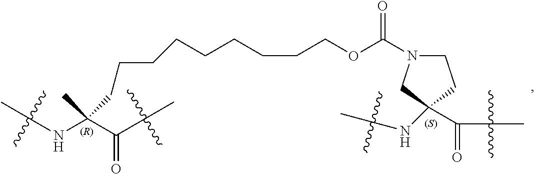

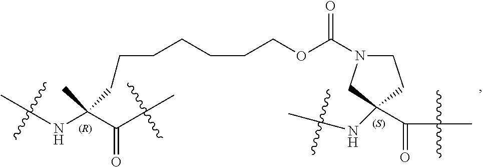

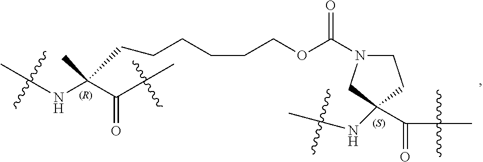

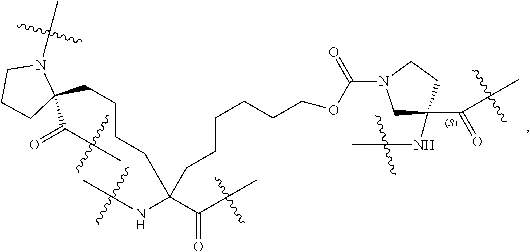

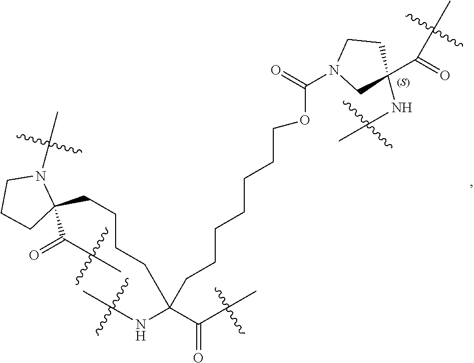

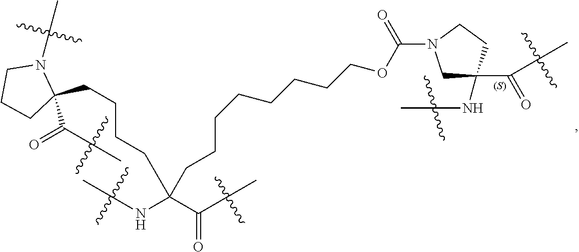

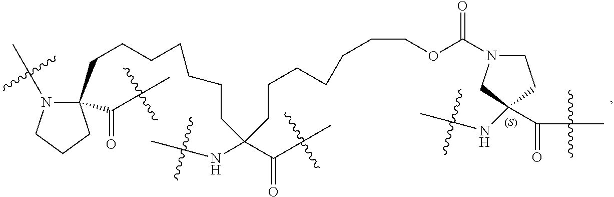

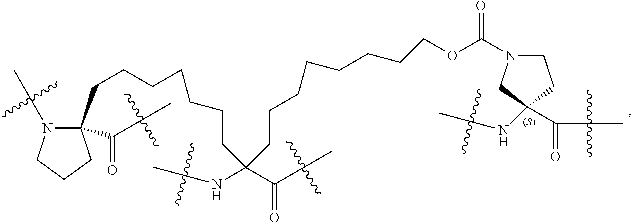

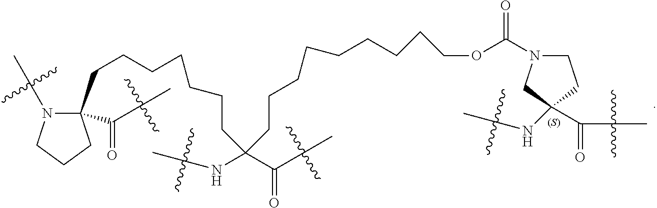

In some embodiments, a staple comprises —S—. In some embodiments, stapling technologies comprise utilization of one or more, e.g., two or more, sulfur-containing moieties. In some embodiments, a stapled peptide comprises cysteine stapling. In some embodiments, two cysteine residues are stapled wherein the —S— moieties of the two cysteine residues are connected optionally through a linker. In some embodiments, a stapled peptide comprises one and no more than one staples from cysteine stapling. In some embodiments, a stapled peptide comprises one and no more than one staples having the structure of

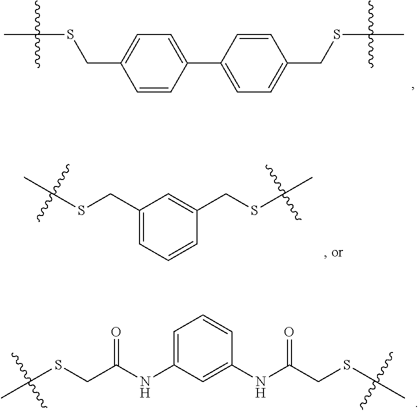 [SMILES: *SCc1ccc(-c2ccc(CS*)cc2)cc1 ; *SCc1cccc(CS*)c1 ; *SCC(=O)Nc1cccc(NC(=O)CS*)c1]

In some embodiments, a stapled peptide comprises one and no more than one staples having the structure of

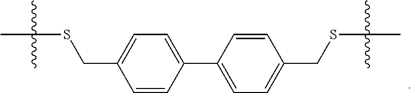 [SMILES: *SCc1ccc(-c2ccc(CS*)cc2)cc1]

In some embodiments, a stapled peptide comprises one and no more than one staples having the structure of

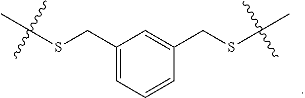 [SMILES: *SCc1cccc(CS*)c1]

In some embodiments, a stapled peptide comprises one and no more than one staples having the structure of

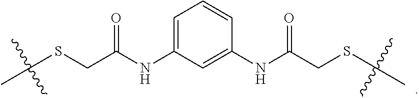 [SMILES: *SCC(=O)Nc1cccc(NC(=O)CS*)c1]

In some embodiments, a stapled peptide comprises no staples having the structure of

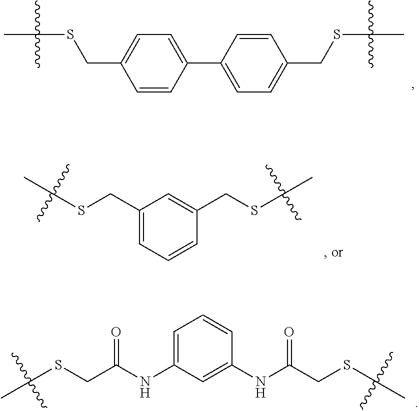 [SMILES: *SCc1ccc(-c2ccc(CS*)cc2)cc1 ; *SCc1cccc(CS*)c1 ; *SCC(=O)Nc1cccc(NC(=O)CS*)c1]

In some embodiments, a stapled peptide comprises no staples having the structure of

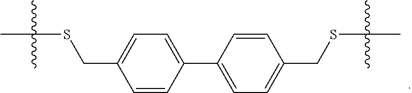 [SMILES: *SCc1ccc(-c2ccc(CS*)cc2)cc1]

In some embodiments, a stapled peptide comprises no staples having the structure of

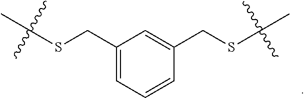 [SMILES: *SCc1cccc(CS*)c1]

In some embodiments, a stapled peptide comprises no staples having the structure of

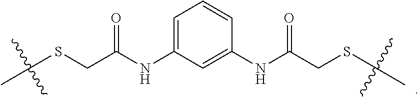 [SMILES: *SCC(=O)Nc1cccc(NC(=O)CS*)c1]

In some embodiments, the present disclosure provides useful technologies relating to cysteine stapling. Among other things, the present disclosure appreciates that peptides amenable to cysteine stapling and/or comprising one or more cysteine staples, can be produced and/or assessed in a biological system. The present disclosure further appreciates that certain such systems permit development, production, and/or assessment of cysteine stapled peptides having a range of different structures (e.g., different amino acid sequences), and in fact can provide a user with complete control over selection and implementation of amino acid sequences to be incorporated into stapled peptides.

Cysteine stapling, as described herein, involves linking one cysteine residue to another cysteine residue, where the resulting bond is not through the peptide backbone between the linked cysteine residues.

In some embodiments, a stapled peptide as described herein comprises a staple which staple is L^{s}, wherein:
L^{s} is -L^{s1}-S-L^{s2}-S-L^{s3}-;
L^{s1} and L^{s3} are each independently L;
L^{s2} is L and comprises at least one —C(O)—; and
each L is independently a covalent bond, or an optionally substituted, bivalent C₁-C₂₅ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond; or
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, L is independently a bivalent C₁-C₂₅ aliphatic group. In some embodiments, L is independently a bivalent C₁-C₂₀ aliphatic group. In some embodiments, L is independently a bivalent C₁-C₁₀ aliphatic group. In some embodiments, L is independently a bivalent C₁-C₅ aliphatic group. In some embodiments, L is independently a bivalent C₁ aliphatic group. In some embodiments, L is —CH₂.

In some embodiments, L^{s1} is —CH₂—. In some embodiments, L^{s3} is —CH₂—. In some embodiments, L^{s1} and L^{s3} are both —CH₂—. In some embodiments, L^{s} is —CH₂—S-L^{s2}-S—CH₂—.

In some embodiments, L^{s2} comprises —C(R′)₂-L′-C(R′)₂—, wherein L′ is described in the present disclosure. In some embodiments, L^{s2} is -L^{s1}-C(O)Q-L′-QC(O)-L^{x1}-, wherein each variable is independently as described in the present disclosure. In some embodiments, L^{s2} is —CH₂C(O)Q-L′-QC(O)CH₂—, wherein each —CH₂— is independently and optionally substituted. In some embodiments, L^{s2} is —CH₂C(O)Q-L′-QC(O)CH₂—.

In some embodiments, L^{s2} In some embodiments, L^{s2} is L and comprises at least one —C(O)—. In some embodiments, L^{s2} is L and comprises at least two —C(O)—. In some embodiments, L^{s2} is L and comprises at least one —C(O)Q-, wherein Q is selected from the group consisting of: a covalent bond, —N(R′)—, —O—, and —S—. In some embodiments, L^{s2} is L and comprises at least one —C(O)Q-, wherein Q is selected between —N(R′)— and —O—. In some embodiments, L^{s2} is L and comprises at least two —C(O)Q-, wherein Q is selected from the group consisting of: —N(R′)—, —O—, and —S—. In some embodiments, L^{s2} is L and comprises at least two —C(O)Q-, wherein Q is selected between —N(R′)— and —O—. In some embodiments, L^{s2} is L and comprises at least one —C(O)N(R′)—. In some embodiments, L^{s2} is L and comprises at least two —C(O)N(R′)—. In some embodiments, L^{s2} is L and comprises at least one —C(O)O—. In some embodiments, L^{s2} is L and comprises at least two —C(O)O—.

In some embodiments, L^{s2} comprises -Q-L′-Q-, wherein Q is independently selected from the group consisting of: —N(R′)—, —O—, and —S, wherein L′ is described in the present disclosure.

In some embodiments, L^{s2} comprises -Q-L′-Q-, wherein Q is independently selected between —N(R′)— and —O—, wherein L′ is described in the present disclosure. In some embodiments, L^{s2} comprises —C(O)Q-L′-QC(O)—, wherein Q is independently selected from the group consisting of: —N(R′)—, —O—, and —S, wherein L′ is described in the present disclosure. In some embodiments, L^{s2} comprises —C(O)Q-L′-QC(O)—, wherein Q is independently selected between —N(R′)— and —O, wherein L′ is described in the present disclosure. In some embodiments, L^{s2} comprises —C(R′)₂C(O)Q-L′-QC(O)C(R′)₂—, wherein Q is independently selected from the group consisting of: —N(R′)—, —O—, and —S, wherein L′ is described in the present disclosure. In some embodiments, L^{s2} comprises —C(R′)₂C(O)Q-L′-QC(O)C(R′)₂—, wherein Q is independently selected between —N(R′)— and —O, wherein L′ is described in the present disclosure.

In some embodiments, L^{s2} comprises —N(R′)-L′—N(R′)—, wherein L′ is described in the present disclosure. In some embodiments, L^{s2} comprises —C(O)N(R′)-L′—N(R′)C(O)—, wherein L′ is described in the present disclosure. In some embodiments, L^{s2} is —C(R′)₂C(O)N(R′)-L′—N(R′)C(O)C(R′)₂—, wherein L′ is described in the present disclosure.

In some embodiments, L^{s2} comprises —O(R′)-L′—O(R′)—, wherein L′ is described in the present disclosure. In some embodiments, L^{s2} comprises —C(O)O-L′—OC(O)—, wherein L′ is described in the present disclosure. In some embodiments, L^{s2} is —C(R′)₂C(O)O-L′—OC(O)C(R′)₂—, wherein L′ is described in the present disclosure.

In some embodiments, R′ is an optionally substituted C₁-30 aliphatic. In some embodiments, R′ is an optionally substituted C₁₋₁₅ aliphatic. In some embodiments, R′ is an optionally substituted C₁₋₁₀ aliphatic. In some embodiments, R′ is an optionally substituted C₁₋₅ aliphatic. In some embodiments, R′ is hydrogen.

In some embodiments, L′ is optionally substituted bivalent C₁-C₁₉ aliphatic. In some embodiments, L′ is optionally substituted bivalent C₁-C₁₅ aliphatic. In some embodiments, L′ is optionally substituted bivalent C₁-C₁₀ aliphatic. In some embodiments, L′ is optionally substituted bivalent C₁-C₉ aliphatic. In some embodiments, L′ is optionally substituted bivalent C₁-C₅ aliphatic. In some embodiments, L′ is optionally substituted bivalent C₁-C₇ aliphatic. In some embodiments, L′ is optionally substituted bivalent C₁-C₆ aliphatic. In some embodiments, L′ is optionally substituted bivalent C₁-C₅ aliphatic. In some embodiments, L′ is optionally substituted bivalent C₁-C₃ aliphatic. In some embodiments, L′ is optionally substituted bivalent C₁-C₂ aliphatic. In some embodiments, L′ is optionally substituted bivalent C₁ aliphatic. In some embodiments, L′ is —CH₂—. In some embodiments, L′ is —(CH₂)₂—. In some embodiments, L′ is —(CH₂)₃—. In some embodiments, L′ is —(CH₂)₄—. In some embodiments, L′ is —(CH₂)₅—. In some embodiments, L′ is —(CH₂)₆—. In some embodiments, L′ is —(CH₂)₇—. In some embodiments, L′ is —(CH₂)₈—.

In some embodiments, L′ is optionally substituted bivalent C₆₋₂₀ aryl ring. In some embodiments, L′ is optionally substituted bivalent C₆₋₁₄ aryl ring. In some embodiments, L′ is optionally substituted bivalent C₆₋₁₀ aryl ring. In some embodiments, L′ is optionally substituted bivalent C₆ aryl ring. In some embodiments, L′ is bivalent C₆ aryl substituted with at least one halogen. In some embodiments, L′ is bivalent C₆ aryl substituted with at least two halogen. In some embodiments, L′ is bivalent C₆ aryl substituted with at least three halogen. In some embodiments, L′ is bivalent C₆ aryl substituted with four halogen. In some embodiments, L′ is bivalent C₆ aryl substituted with at least one fluorine. In some embodiments, L′ is bivalent C₆ aryl substituted with at least two fluorine. In some embodiments, L′ is bivalent C₆ aryl substituted with at least three fluorine. In some embodiments, L′ is bivalent C₆ aryl substituted with four fluorine. In some embodiments, L′ is bivalent C₆ aryl substituted with at least one chlorine. In some embodiments, L′ is bivalent C₆ aryl substituted with at least two chlorine. In some embodiments, L′ is bivalent C₆ aryl substituted with at least three chlorine. In some embodiments, L′ is bivalent C₆ aryl substituted with four chlorine. In some embodiments, L′ is bivalent C₆ aryl substituted at with least one —O(CH₂)₀₋₄CH₃. In some embodiments, L′ is bivalent C₆ aryl substituted with at least two —O(CH₂)₀₋₄CH₃. In some embodiments, L′ is bivalent C₆ aryl substituted with at least three —O(CH₂)₀₋₄CH₃. In some embodiments, L′ is bivalent C₆ aryl substituted with four —O(CH₂)₀₋₄CH₃.

In some embodiments, L′ is bivalent 5-20 membered heteroaryl ring having 1-10 heteroatoms independently selected from oxygen, nitrogen, sulfur, phosphorus and silicon. In some embodiments, L′ is bivalent 5−6 membered heteroaryl ring having 1-4 heteroatoms independently selected from oxygen, nitrogen, sulfur, phosphorus and silicon. In some embodiments, L′ is bivalent 5−6 membered heteroaryl ring having 1-4 heteroatoms independently selected from oxygen, nitrogen, and sulfur. In some embodiments, L′ is bivalent 6 membered heteroaryl ring having 1-2 heteroatoms independently selected from oxygen, nitrogen, and sulfur. In some embodiments, L′ is bivalent 6 membered heteroaryl ring having 2 nitrogen.

In some embodiments, L′ is optionally substituted bivalent C₃₋₂₀ cycloaliphatic ring. In some embodiments, L′ is optionally substituted bivalent C₃₋₁₅ cycloaliphatic ring. In some embodiments, L′ is optionally substituted bivalent C₃₋₁₀ cycloaliphatic ring. In some embodiments, L′ is optionally substituted bivalent C₃₋₉ cycloaliphatic ring. In some embodiments, L′ is optionally substituted bivalent C₃₋₈ cycloaliphatic ring. In some embodiments, L′ is optionally substituted bivalent C₃₋₇ cycloaliphatic ring. In some embodiments, L′ is optionally substituted bivalent C₃₋₆ cycloaliphatic ring. In some embodiments, L′ is optionally substituted bivalent C₃-s cycloaliphatic ring. In some embodiments, L′ is optionally substituted bivalent C₃₋₄ cycloaliphatic ring. In some embodiments, L′ is optionally substituted bivalent C₃ cycloaliphatic ring. In some embodiments, L′ is optionally substituted bivalent C₄ cycloaliphatic ring. In some embodiments, L′ is optionally substituted bivalent C₅ cycloaliphatic ring. In some embodiments, L′ is optionally substituted bivalent C₅ cycloalkyl ring. In some embodiments, L′ is optionally substituted bivalent C₅ cycloalkenyl ring. In some embodiments, L′ is optionally substituted bivalent C₆ cycloaliphatic ring. In some embodiments, L′ is optionally substituted bivalent C₆ cycloalkyl ring.

In some embodiments, L^{s2} comprises —N(R′)-L′—N(R′)— and L′ is a covalent bond. In some embodiments L^{s2} comprises —N(R)—N(R)—, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments L^{s2} comprises —N(R)—N(R)—, wherein:
each R is independently optionally substituted C₁₋₃₀ aliphatic; or
two or more R groups on two or more atoms are optionally and independently taken together with their intervening atoms to form an optionally substituted, 3-30 membered monocyclic ring.

In some embodiments, L^{s2} is a staple selected from the group consisting of:

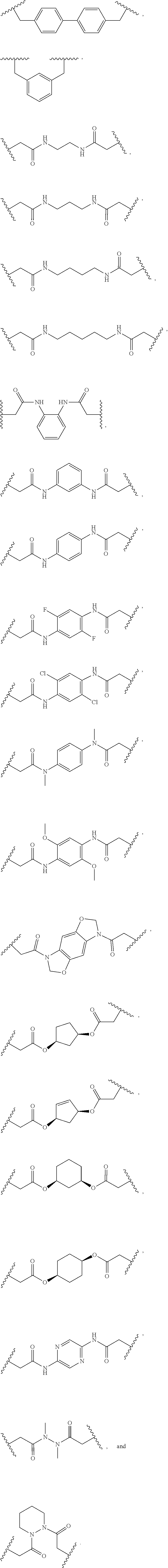

As those skilled in the art will appreciate, provided technologies can be utilized to prepare collection of peptides using non-cysteine residues and suitable chemistry therefor. For example, in some embodiments, cysteine stapling is replaced with lysine stapling, wherein the cysteine residues for cysteine stapling are replaced with lysine residues for lysine stapling (e.g., using agents that can crosslink two lysine residues, for example, through reactions with side chain amino groups). In some embodiments, for lysine stapling, RE in various formulae is or comprises an activated carboxylic acid group (e.g., NHS ester group), an imidoester group, etc. Suitable reagents are widely known in the art including many commercially available ones. In some embodiments, cysteine stapling is replaced with methionine stapling. In some embodiments, cysteine residues for cysteine stapling are replaced with methionine residues for methionine stapling. In some embodiments, cysteine stapling is replaced with tryptophan stapling. In some embodiments, cysteine residues for cysteine stapling are replaced with tryptophan residues for tryptophan stapling. As those skilled in the art will appreciate, various technologies (e.g., reagents, reactions, etc.) are described in the art and can be utilized in accordance with the present disclosure for, e.g., methionine stapling, tryptophan stapling, etc. In some embodiments, such stapling can be performed using reagents having various formulae described herein, wherein R^{E} is or comprises a group that are suitable for methionine and/or tryptophan stapling. In some embodiments, stapling may be performed using one residue at a first position, and a different residue at a second position. Useful reagents for such stapling may comprise a first reactive group for stapling at a first position (e.g., through a first R^{E}), and a second reactive group for stapling at a second position (e.g., through a second R^{E}).

In some embodiments, for various types of stapling (e.g., cysteine stapling, or non-cysteine stapling), stapling is between residues (e.g., cysteine residues for cysteine stapling) separated by two residues (i+3 stapling). In some embodiments, stapling is between residues separated by three residues (i+4 stapling). In some embodiments, stapling is between residues separated by six residues (i+7 stapling).

As appreciated by those skilled in the art, in some embodiments, more than two residues can be stapled at the same time. For example, in some embodiments, three or more cysteines are stapled using crosslinking reagents containing three or more reactive groups (e.g., R^{E} groups).

In some embodiments, as described herein, the present disclosure provides useful technologies relating to non-cysteine stapling. Among other things, the present disclosure appreciates that peptides amenable to cysteine stapling and/or comprising one or more non-cysteine staples, can have its cysteine residues and cysteine staple replaced with other amino acids and staples described herein (e.g. hydrocarbon and other non-hydrocarbon amino acid and staples). In some embodiments, the resulting non-cysteine stapled peptide maintains the same or similar interaction with a target of interest when compared to a reference cysteine stapled peptide.

Certain useful agents (peptides prior to stapling and stapled peptides post stapling) and compositions thereof are presented in Table E3 as examples, which includes various amino acid residues and N- and C-terminus capping groups for various positions as examples; also illustrated are various stapling patterns, e.g., X¹—X⁴—X¹¹, X³—X¹⁰, X⁴—X¹¹, X¹—X⁴ and X¹⁰—X¹⁴, X¹—X⁴ and X⁷—X¹⁴ etc. In some embodiments, a provided agent has a structure selected from Table E3 or a salt thereof. In some embodiments, a provided composition is a composition described in Table E3. As shown, e.g., in Tables E1 and E2 and the Figures, provided technologies can deliver improved useful properties and/or activities. For example, in some embodiments, a provided agent is a stapled peptide having the structure of

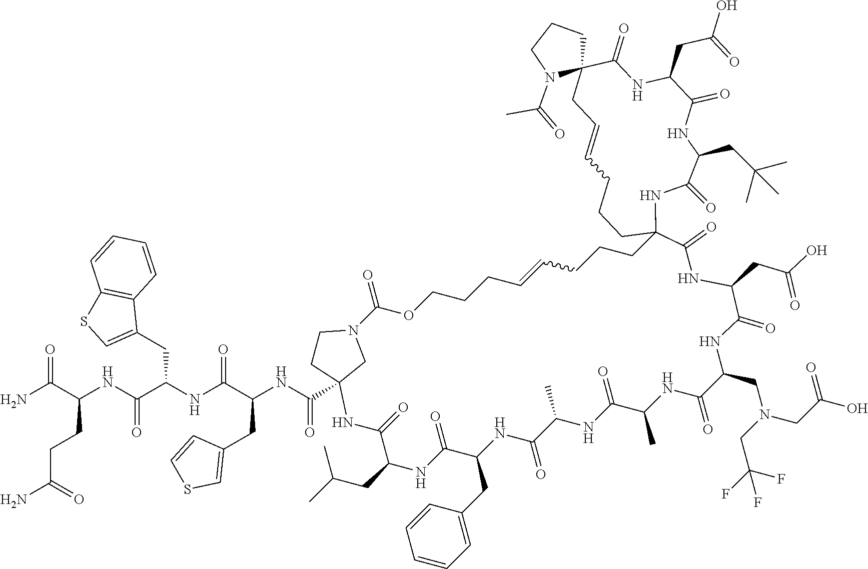 [SMILES: CC(=O)N1CCC[C@]12CC=CCCCC1(CCCC=CCCCOC(=O)N3CC[C@@](C(=O)N[C@@H](Cc4ccsc4)C(=O)N[C@@H](Cc4csc5ccccc45)C(=O)N[C@@H](CCC(N)=O)C(N)=O)(C3)NC(=O)[C@H](CC(C)C)NC(=O)[C@H](Cc3ccccc3)NC(=O)[C@H](C)NC(=O)[C@H](C)NC(=O)[C@H](CN(CC(=O)O)CC(F)(F)F)NC(=O)[C@H](CC(=O)O)NC1=O)NC(=O)[C@H](CC(C)(C)C)NC(=O)[C@H](CC(=O)O)NC2=O]

or a salt thereof. In some embodiments, a provided agent is a stapled peptide having the structure of

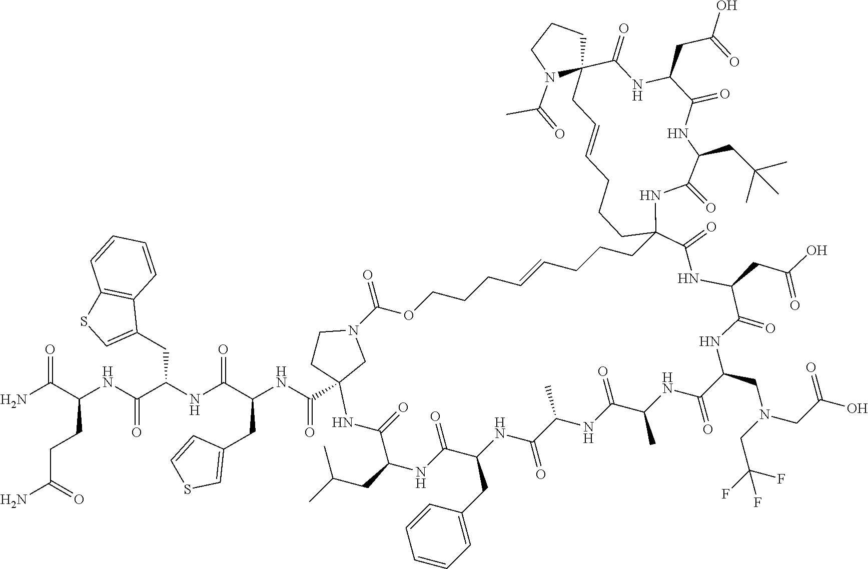 [SMILES: CC(=O)N1CCC[C@]12C/C=C/CCCC1(CCC/C=C/CCCOC(=O)N3CC[C@@](C(=O)N[C@@H](Cc4ccsc4)C(=O)N[C@@H](Cc4csc5ccccc45)C(=O)N[C@@H](CCC(N)=O)C(N)=O)(C3)NC(=O)[C@H](CC(C)C)NC(=O)[C@H](Cc3ccccc3)NC(=O)[C@H](C)NC(=O)[C@H](C)NC(=O)[C@H](CN(CC(=O)O)CC(F)(F)F)NC(=O)[C@H](CC(=O)O)NC1=O)NC(=O)[C@H](CC(C)(C)C)NC(=O)[C@H](CC(=O)O)NC2=O]

or a salt thereof. In some embodiments, a provided agent is a stapled peptide having the structure of

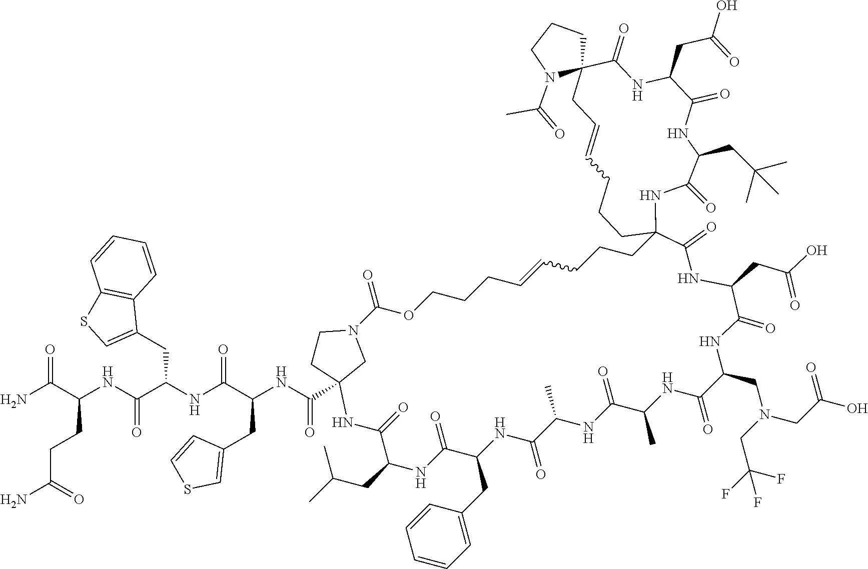 [SMILES: CC(=O)N1CCC[C@]12CC=CCCCC1(CCCC=CCCCOC(=O)N3CC[C@@](C(=O)N[C@@H](Cc4ccsc4)C(=O)N[C@@H](Cc4csc5ccccc45)C(=O)N[C@@H](CCC(N)=O)C(N)=O)(C3)NC(=O)[C@H](CC(C)C)NC(=O)[C@H](Cc3ccccc3)NC(=O)[C@H](C)NC(=O)[C@H](C)NC(=O)[C@H](CN(CC(=O)O)CC(F)(F)F)NC(=O)[C@H](CC(=O)O)NC1=O)NC(=O)[C@H](CC(C)(C)C)NC(=O)[C@H](CC(=O)O)NC2=O]

or a salt thereof. In some embodiments, a provided agent is a stapled peptide having the structure of

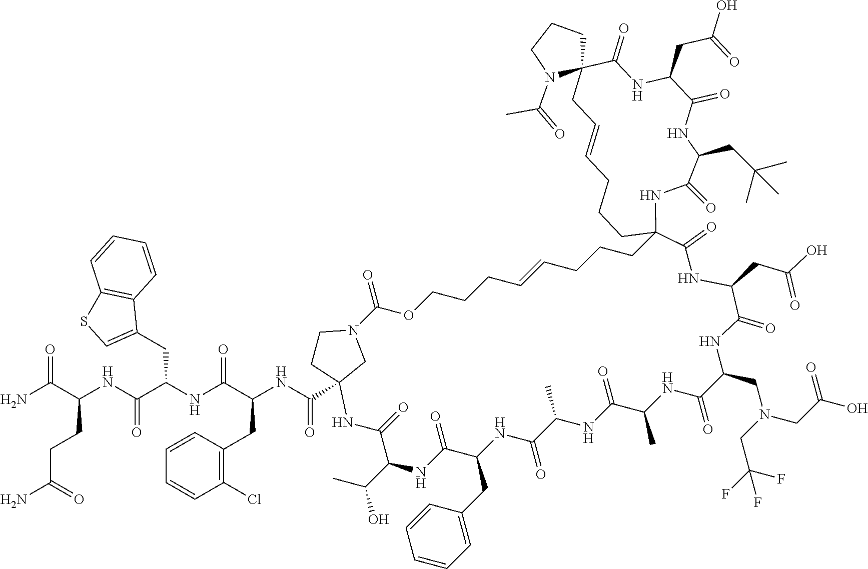 [SMILES: CC(=O)N1CCC[C@]12C/C=C/CCCC1(CCC/C=C/CCCOC(=O)N3CC[C@@](C(=O)N[C@@H](Cc4ccccc4Cl)C(=O)N[C@@H](Cc4csc5ccccc45)C(=O)N[C@@H](CCC(N)=O)C(N)=O)(C3)NC(=O)[C@H]([C@@H](C)O)NC(=O)[C@H](Cc3ccccc3)NC(=O)[C@H](C)NC(=O)[C@H](C)NC(=O)[C@H](CN(CC(=O)O)CC(F)(F)F)NC(=O)[C@H](CC(=O)O)NC1=O)NC(=O)[C@H](CC(C)(C)C)NC(=O)[C@H](CC(=O)O)NC2=O]

or a salt thereof. In some embodiments, a provided agent is a stapled peptide having the structure of

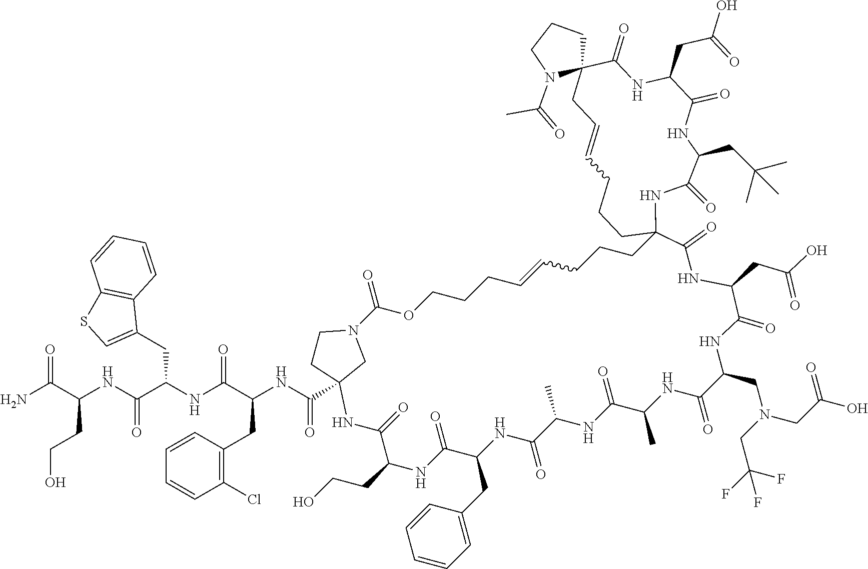 [SMILES: CC(=O)N1CCC[C@]12CC=CCCCC1(CCCC=CCCCOC(=O)N3CC[C@@](C(=O)N[C@@H](Cc4ccccc4Cl)C(=O)N[C@@H](Cc4csc5ccccc45)C(=O)N[C@@H](CCO)C(N)=O)(C3)NC(=O)[C@H](CCO)NC(=O)[C@H](Cc3ccccc3)NC(=O)[C@H](C)NC(=O)[C@H](C)NC(=O)[C@H](CN(CC(=O)O)CC(F)(F)F)NC(=O)[C@H](CC(=O)O)NC1=O)NC(=O)[C@H](CC(C)(C)C)NC(=O)[C@H](CC(=O)O)NC2=O]

or a salt thereof. In some embodiments, a provided agent is a stapled peptide having the structure of

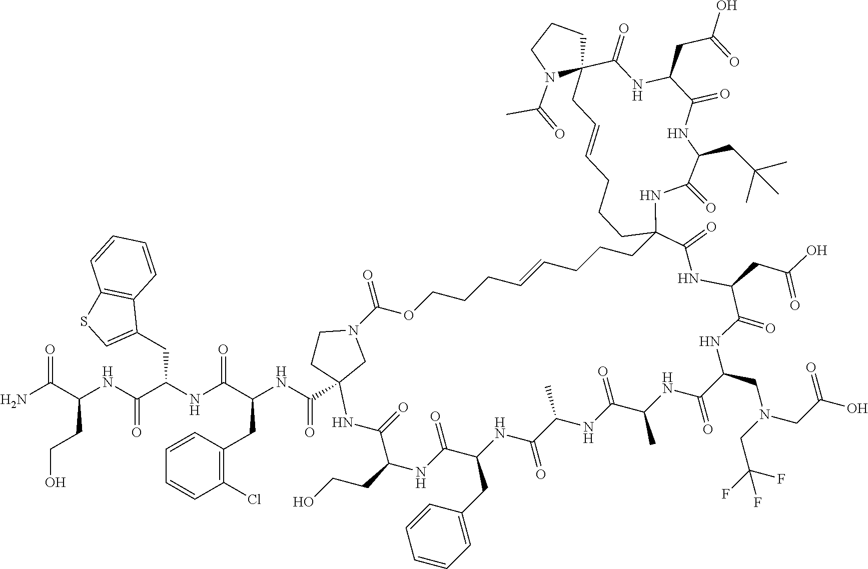 [SMILES: CC(=O)N1CCC[C@]12C/C=C/CCCC1(CCC/C=C/CCCOC(=O)N3CC[C@@](C(=O)N[C@@H](Cc4ccccc4Cl)C(=O)N[C@@H](Cc4csc5ccccc45)C(=O)N[C@@H](CCO)C(N)=O)(C3)NC(=O)[C@H](CCO)NC(=O)[C@H](Cc3ccccc3)NC(=O)[C@H](C)NC(=O)[C@H](C)NC(=O)[C@H](CN(CC(=O)O)CC(F)(F)F)NC(=O)[C@H](CC(=O)O)NC1=O)NC(=O)[C@H](CC(C)(C)C)NC(=O)[C@H](CC(=O)O)NC2=O]

or a salt thereof.
In some embodiments, a double bond of a (i, i+3) staple is E. In some embodiments, a double bond of a (i, i+3) staple is Z. In some embodiments, a double bond of a (i, i+4) staple is E. In some embodiments, a double bond of a (i, i+4) staple is Z. In some embodiments, a double bond of a (i, i+7) staple is E. In some embodiments, a double bond of a (i, i+7) staple is Z. In some embodiments, both double bonds are E. In some embodiments, both double bonds are Z. In some embodiments, a (i, i+3) staple is E, and the other is Z. In some embodiments, a (i, i+3) staple is Z, and the other is E.

Agents, e.g., peptides including stapled peptides, can contain various numbers of amino acid residues. In some embodiments, a length of a peptide agent is about 5-20, 5-19, 5-18, 5-17, 5-16, 5-15, 10-20, 10-19, 10-18, 10-17, 10-16, 10-15, 11-20, 11-19, 11-18, 11-17, 11-16, 11-15, 12-20, 12-19, 12-18, 12-17, 12-16, 12-15, 13-20, 13-19, 13-18, 13-17, 13-16, 13-15, 14-20, 14-19, 14-18, 14-17, 14-16, 14-15, or about 2, 3, 4, 5, 6, 7, 8, 9, 10, 11, 12, 13, 14, 15, 16, 17, 18, 19, or 20 amino acid residues. In some embodiments, a length is about 10 amino acid residues. In some embodiments, a length is about 11 amino acid residues. In some embodiments, a length is about 12 amino acid residues. In some embodiments, a length is about 13 amino acid residues. In some embodiments, a length is about 14 amino acid residues. In some embodiments, a length is about 15 amino acid residues. In some embodiments, a length is about 16 amino acid residues. In some embodiments, a length is about 17 amino acid residues. In some embodiments, a length is about 18 amino acid residues. In some embodiments, a length is about 19 amino acid residues. In some embodiments, a length is about 20 amino acid residues.

Certain useful staples are described in the “Agents” section, below.

### Beta-Catenin

Among other things, the present disclosure provides technologies for modulating one or more beta-catenin functions. In some embodiments, the present disclosure provides useful technologies for inhibiting one or more beta-catenin functions that are associated with cancer or hyperplasia. In some embodiments, provided technologies are useful for preventing and treating conditions, disorders or diseases whose prevention and/or treatment will benefits from inhibition of beta-catenin. In some embodiments, a condition, disorder or disease is cancer.

Beta-catenin is reported to have various functions. For example, it can regulate and coordinate transcription of various genes. It is reported that high beta-catenin activity and/or expression levels may contribute to the development various conditions, disorders or diseases including cancer. Mutations and overexpression of beta-catenin are reported to be associated with conditions, disorders or diseases including many cancers including colorectal cancer, lung cancer, and breast cancer. Dysregulation of the Wnt/β-catenin signaling pathway has reportedly been linked to a number of conditions, disorders or diseases, including neurodegenerative diseases, psychiatric diseases, cancers, asthma, and even wound healing. Agents that can modulate beta-catenin functions are useful for various purposes including preventing and/or treating various conditions, disorders or diseases associated with beta-catenin.

### Binding Sites

Beta-catenin may interact with various agents at various binding sites each independently comprising a set of amino acid residues that interact with binding agents. For example, certain binding sites are utilized for beta-catenin interactions with Axin, APC, C-cadherin, E-cadherin, TCF3, and Bcl9. For interactions with TCF3, it has been reported that two or more binding sites may be utilized simultaneously to interact with different portions of TCF3. See, e.g., Graham et al. Cell, Vol. 103, 885-896, 2000.

In some embodiments, provided agents bind to beta-catenin at a unique binding site. In some embodiments, provided agents interact with beta-catenin at a set of amino acid residues that are different from previously reported binding sites, e.g., those for Axin, APC, C-cadherin, E-cadherin, TCF3 or Bcl9.

For example, in some embodiments, provided agents interact with one or more or all (e.g., about 1-23, 1-20, 1-15, 1-10, 1-5, 5-23, 5-20, 5-15, 5-10, 6-23, 6-20, 6-15, 6-10, 7-23, 7-20, 7-15, 7-10, 8-23, 8-20, 8-15, 8-10, 9-23, 9-20, 9-15, 9-10, 10-23, 10-20, 10-15, 11-23, 11-20, 11-15, 12-23, 12-20, 12-15, 13-23, 13-20, 13-15, 13-23, 14-20, 15-23, 15-20, 16-23, 16-20, 17-23, 17-20, 18-23, or 18-20, or about 1, 2, 3, 4, 5, 6, 7, 8, 9, 10, 11, 12, 13, 14, 15, 16, 17, 18, 19, 20, 21, 22, or 23, etc.) of a set of amino acid residues that are or correspond to amino acid residues in SEQ ID NO: 1, e.g., in some embodiments, the following amino acid residues of SEQ ID NO: 1: A305, Y306, G307, N308, Q309, K312, R342, K345, V346, V349, Q375, R376, Q379, N380, L382, W383, R386, N387, D413, N415, V416, T418, and C419. In some embodiments, a set of amino acid residues are or correspond to amino acid residues A305, Y306, G307, N308, Q309, K312, R342, K345, V346, V349, Q375, Q379, N380, L382, W383, R386, N387, D413, N415, V416, T418, and C419 of SEQ ID NO: 1. In some embodiments, a set of amino acid residues are or correspond to amino acid residues A305, Y306, G307, N308, Q309, K312, K345, V346, V349, Q379, N380, L382, W383, R386, N387, D413, N415, V416, T418, and C419 of SEQ ID NO: 1. In some embodiments, a set of amino acid residues are or correspond to amino acid residues G307, K312, K345, W383, N387, D413, and N415 of SEQ ID NO: 1. In some embodiments, a set of amino acid residues are or correspond to amino acid residues G307, K312, K345, W383, and N387 of SEQ ID NO: 1. In some embodiments, a set of amino acid residues are or correspond to amino acid residues Y306, G307, K312, R386 and N387 of SEQ ID NO: 1. In some embodiments, provided agents interact with Y306 or an amino acid residue corresponding thereto. In some embodiments, provided agents interact with G307 or an amino acid residue corresponding thereto. In some embodiments, provided agents interact with K312 or an amino acid residue corresponding thereto. In some embodiments, provided agents interact with K345 or an amino acid residue corresponding thereto. In some embodiments, provided agents interact with R386 or an amino acid residue corresponding thereto. In some embodiments, provided agents interact with W383 or an amino acid residue corresponding thereto. In some embodiments, provided agents interact with N387 or an amino acid residue corresponding thereto.

In some embodiments, a present agent interacts with a polypeptide whose sequence comprises or is SEQ ID NO: 2:

(SEQ ID NO: 2)
SVLFYAITTLHNLLLHQEGAKMAVRLAGGLQKMVALLNKTNVKFLAITTD
CLQILAYGNQESKLIILASGGPQALVNIMRTYTYEKLLWTTSRVLKVLSV
CSSNKPAIVEAGGMQALGLHLTDPSQRLVQNCLWTLRNLSDAATKQEGME
GLLGTLVQLLGSDDINVVTCAAGILSNLTCNNYKNKMMVCQVGGIEALVR
T.

In some embodiments, all amino acid residues that interact with a provided agent is with SEQ ID NO: 2. In some embodiments, amino acid residues that interact with a provided agent (e.g., one or more amino acid residues in an agent) interacts with an agent through hydrogen bonding, hydrophobic interactions or salt bridge. As appreciated by those skilled in the art, when two amino acid residues interacting with each other, they are typically within a certain range of distances when, e.g., assessed using crystallography, NMR, etc.

In some embodiments, certain amino acid residues reported to interact with one or more polypeptides are not significantly involved in interactions between provided and beta-catenin. In some embodiments, provided agents do not interact with an Axin binding site. In some embodiments, provided agents do not interact with a Bcl9 binding site. In some embodiments, provided agents do not interact with one or more or all of amino acid residues that are or correspond to N426, C429, K435, R469, H470, S473, R474, K508 and N516 of SEQ ID NO: 1. In some embodiments, provided agents do not interact with N426 or an amino acid residue corresponding thereto. In some embodiments, provided agents do not interact with C429 or an amino acid residue corresponding thereto. In some embodiments, provided agents do not interact with K435 or an amino acid residue corresponding thereto. In some embodiments, provided agents do not interact with R469 or an amino acid residue corresponding thereto. In some embodiments, provided agents do not interact with H470 or an amino acid residue corresponding thereto. In some embodiments, provided agents do not interact with S473 or an amino acid residue corresponding thereto. In some embodiments, provided agents do not interact with R474 or an amino acid residue corresponding thereto. In some embodiments, provided agents do not interact with K508 or an amino acid residue corresponding thereto. In some embodiments, provided agents do not interact with N516 or an amino acid residue corresponding thereto.

In some embodiments, mutation of one or more amino acid residues outside of SEQ ID NO: 2 in beta-catenin does not significant/y (e.g., not exceeding 30%, 40%, 50%, 60%, 70%, 75%, 80%, 85%, 90% or more) reduce interactions of beta-catenin with a provided agent. In some embodiments, mutation of one or more or all of amino acid residues that are or correspond to N426, C429, K435, R469, H470, S473, R474, K508 and N516 of SEQ ID NO: 1 does not significantly reduce interactions of beta-catenin with a provided agent. In some embodiments, mutation of N426 or an amino acid residue corresponding thereto does not significantly reduce interaction of beta-catenin with an agent. In some embodiments, mutation of Q379 or an amino acid residue corresponding thereto (e.g., to Ala, Glu, Phe, Trp, etc.) does not significantly reduce interaction of beta-catenin with an agent.

In some embodiments, an agent binds to a TCF site of beta-catenin. In some embodiments, an agent interacts with one or more but not all amino acid residues that interact with TCF. In some embodiments, an agent interacts with one or more but not all amino acid residues that interact with an extended region of XTcf3-CBD. In some embodiments, an agent does not interact with beta-catenin amino acid residues that interact with a beta-hairpin module of XTcf3-CBD. In some embodiments, an agent does not interact with beta-catenin amino acid residues that interact with a helix module of XTcf3-CBD. For certain amino acid residues that interact various modules of XTcF3-CBD, see, e.g., Graham et al. Cell, Vol. 103, 885-896, 2000.

In some embodiments, an agent competes with TCF for beta-catenin binding. In some embodiments, an agent competes with an extended region of TCF (e.g., Ala14-Glu24, or Asp16-Glu24, as described in Graham et al. Cell, Vol. 103, 885-896, 2000) for beta-catenin binding. In some embodiments, compared to an extended region of TCF, an agent does not compete, or competes at a less degree, with Axin for beta-catenin binding. In some embodiments, compared to an extended region of TCF, an agent does not compete, or competes at a less degree, with Bcl9 for beta-catenin binding. In some embodiments, compared to an extended region of TCF, an agent does not compete, or competes at a less degree, with a beta-hairpin module of XTcf3-CBD for beta-catenin binding. In some embodiments, compared to an extended region of TCF, an agent does not compete, or competes at a less degree, with a helix module of XTcf3-CBD for beta-catenin binding. In some embodiments, an agent competes with E-cadherin for beta-catenin binding.

In some embodiments, the present disclosure provides complexes of peptides (e.g., polypeptides whose sequences are or comprises SEQ ID NO: 1 or 2) and provided agents. In some embodiments, in such complexes polypeptides and provided agents interact with one or more or all amino acid residues as described herein, and optionally do not interact with one or more or all amino acid residues as described herein.

In some embodiments, the present disclosure provides complexes comprising a provided agent and a beta-catenin polypeptide or a portion thereof. In some embodiments, a portion thereof comprises one or more or all of the interacting residues as described herein. In some embodiments, an agent and a beta-catenin polypeptide or a portion thereof interact with other at one or more or all of the interacting residues.

### Certain Agents

In some embodiments, the present disclosure provides an agent having the structure of formula I:

R^{N}-L^{P1}-L^{AA1}-L^{P2}L^{AA2}-L^{P3}-L^{AA3}-L^{P4}-L^{AA4}-L^{P5}-L^{AA5}-L^{P6}-L^{AA6}-L^{P7}-R^{C} I

or a salt thereof, wherein:
R^{N} is a peptide, an amino protecting group or R′-L^{RN}-.
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
   a first R′ group and a second R′ group which are taken together to form -L^{s}- which is bonded to the atom to which a first R′ group is attached and the atom to which a second R′ group is attached; and
   a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L^{s3}-, wherein each L^{s1}, L^{s2} and L^{s3} is independently L;
L^{AA1} is an amino acid residue that comprises a side chain comprising an acidic or polar group;
L^{AA2} is an amino acid residue that comprises a side chain comprising an acidic or polar group;
L^{AA3} is an amino acid residue;
L^{AA4} is an amino acid residue that comprises a side chain comprising an optionally substituted aromatic group;
L^{AA5} is an amino acid residue that comprises a side chain comprising an optionally substituted aromatic group;
L^{AA6} is an amino acid residue that comprises a side chain comprising an optionally substituted aromatic group;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

In some embodiments, the present disclosure provides an agent having the structure of formula I:

R^{N}-L^{P1}-L^{AA1}-L^{P2}L^{AA2}-L^{P3}-L^{AA3}-L^{P4}-L^{AA4}-L^{P5}-L^{AA5}-L^{P6}-L^{AA6}-L^{P7}-R^{C} I

or a salt thereof, wherein:
R^{N} is a peptide, an amino protecting group or R′-L^{RN}-;
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
   a first R′ group and a second R′ group which are taken together to form -L^{s}- which is bonded to the atom to which a first R′ group is attached and the atom to which a second R′ group is attached; and
   a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L^{s3}-, wherein each LSL, L^{s2} and L^{s3} is independently L;
L^{AA1} is L^{AR}, wherein a methylene unit is replaced with —C(R′)(R^{AS})—, wherein R^{AS} is -L^{AS1}-R^{AA1} wherein R^{AA1} is —CO₂R or —SO₂R;
L^{AA2} is L^{AR}, wherein a methylene unit is replaced with —C(R′)(R^{AS})—, wherein R^{AS} is -L^{AS2}-R^{AA2} wherein R^{AA2} is —CO₂R or —SO₂R;
L^{AA3} is L^{AR}, wherein a methylene unit is replaced with —C(R′)(R^{AS})—, wherein R^{AS} is -L^{AS3}-R^{AA3} wherein R^{AA3} is R′;
L^{AA4} is L^{AR}, wherein a methylene unit is replaced with —C(R′)(R^{AS})—, wherein R^{AS} is -L^{AS4}-R^{AA4} wherein R^{AA4} is an optionally substituted group selected from 6-14 membered aryl or 5-14 membered heteroaryl having 1−6 heteroatoms;
L^{AA5} is L^{AR}, wherein a methylene unit is replaced with —C(R′)(R^{AS})—, wherein R^{AS} is -L^{AS5}-R^{AA5}wherein R^{AA5} is an optionally substituted group selected from 6-14 membered aryl or 5-14 membered heteroaryl having 1−6 heteroatoms;
L^{AA6} is L^{AR}, wherein a methylene unit is replaced with —C(R′)(R^{AS})—, wherein R^{AS} is -L^{AS6}R^{AA6}, wherein R^{AA6} is an optionally substituted group selected from 6-14 membered aryl or 5-14 membered heteroaryl having 1−6 heteroatoms;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L^{A}R is independently an optionally substituted, bivalent C₁-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each of L^{AS1}, L^{AS2}, L^{AS3}, L^{AS4}, L^{AS5}, and L^{AS6} is independently L^{AS};
each R^{AS} is independently -L^{AS}-R′;
each L^{AS} is independently an optionally substituted, bivalent C₁-C₁₀ aliphatic or heteroaliphatic group having 1-5 heteroatoms, wherein one or more methylene units of the group optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

In some embodiments, a second R′ group and a third R′ group are attached to the same atom. In some embodiments, none of the first, second and fourth R′ groups are attached to the same atom. In some embodiments, each of the first, second, third and fourth R′ groups is independently attached to a different atom.

In some embodiments, a compound of formula I is a stapled peptide as described herein.

In some embodiments, each L^{s} is independently a staple as described herein. In some embodiments, L^{s}, e.g., L^{s} formed by taking the first and the second R′ groups, has a length of 5-20 (e.g., 5, 6, 7, 8, 9, 10, 11, 12, 13, 14, 15, 16, 17, 18, 19, or 20) atoms. Unless specified otherwise, a length between two connection sites, e.g., of L^{s}, L, etc., is the shortest covalent connection from one site to the other. For example, the length of —CH₂—CH₂— is 2 atoms (—C—C—), the length of 1, 3-phenylene is 3 atoms. In some embodiments, L^{s}, e.g., L^{s} formed by taking the third and the fourth R′ groups, has a length of 5-20 (e.g., 5, 6, 7, 8, 9, 10, 11, 12, 13, 14, 15, 16, 17, 18, 19, or 20) atoms. Those skilled in the art reading the present disclosure will appreciate that staples, e.g., L^{s}, connecting two atoms having a longer distance typically has a longer length than staples connecting two atom having a shorter distance, e.g., (i, i+7) staples typically have longer lengths than (i, i+3) or (i, i+4) staples. In some embodiments, a length is 5 atoms. In some embodiments, a length is 6 atoms. In some embodiments, a length is 7 atoms. In some embodiments, a length is 8 atoms. In some embodiments, a length is 9 atoms. In some embodiments, a length is 10 atoms. In some embodiments, a length is 11 atoms. In some embodiments, a length is 12 atoms. In some embodiments, a length is 13 atoms. In some embodiments, a length is 14 atoms. In some embodiments, a length is 15 atoms. In some embodiments, a length is 16 atoms. In some embodiments, a length is 17 atoms. In some embodiments, a length is 18 atoms. In some embodiments, a length is 19 atoms. In some embodiments, a length is 20 atoms.

### L^{P1}

In some embodiments, L^{P1} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the length of L^{P1} is 2-10 atoms. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms. In some embodiments, one or more methylene units are independently replaced with —N(R′)—, —C(R′)₂—, —C(O)— or —C(O)N(R′)—. In some embodiments, a methylene unit is replace with —N(R′)—. In some embodiments, a methylene unit is replace with —C(R′)₂—. In some embodiments, a methylene unit is replace with —C(O)—. In some embodiments, a methylene unit is replace with —C(O)N(R′)—. In some embodiments, each methylene unit is independently replaced with —N(R′)—, —C(R′)₂— or —C(O)—. In some embodiments, L^{P1} is or comprises an amino acid residue. In some embodiments, L^{P1} is or comprises a peptide.

In some embodiments, L^{P1} is or comprises —[X]ₚ—X¹—, wherein each of p, X and X¹ is independently as described herein, and X¹ is bonded to L^{AA1}. In some embodiments, L^{P1} is or comprises —X¹—.

In some embodiments, L^{P1} comprises a —C(R′)₂— group, wherein one of the R′ groups is a first R′ group of the four. In some embodiments, such a —C(R′)₂— group is of an amino acid residue. In some embodiments, such a —C(R′)₂— group is of X¹. In some embodiments, such a carbon atom is an alpha carbon of an amino acid residue.

### L^{AA1}

In some embodiments, L^{AA1} is amino acid residue. In some embodiments, L^{AA1} is an amino acid residue that comprises a side chain comprising an acidic or polar group. In some embodiments, L^{AA1} is an amino acid residue that comprises a side chain comprising an acidic group.

In some embodiments, L^{AA1} is L^{AR}, wherein a methylene unit is replaced with —C(R′)(R^{AS})—, wherein each variable is independently as described herein. In some embodiments, L^{AA1} is an optionally substituted, bivalent C₁-C₆ (e.g., C₁, C₂, C₃, C₄, C₅, or C₆) aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA1} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, LAA is —N(R′)—C(R′)(R^{AS})—C(O)—, wherein each variable is independently as described herein. In some embodiments, L^{AA1} is —NH—C(R′)(R^{AS})—C(O)—, wherein each variable is independently as described herein.

In some embodiments, L^{AS1} is L^{AS} as described herein. In some embodiments, R^{AA1} is —CO₂R, wherein R is as described herein. In some embodiments, R is H. In some embodiments, L^{AA1} is a residue of an acidic amino acid residue, e.g., Asp, Glu, etc. In some embodiments, L^{AA1} is X² as described herein.

### L^{P2}

In some embodiments, L^{P2} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the length of L^{P2} is 2-10 atoms. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms. In some embodiments, one or more methylene units are independently replaced with —N(R′)—, —C(R′)₂—, —C(O)— or —C(O)N(R′)—. In some embodiments, a methylene unit is replace with —N(R′)—. In some embodiments, a methylene unit is replace with —C(R′)₂—. In some embodiments, a methylene unit is replace with —C(O)—. In some embodiments, a methylene unit is replace with —C(O)N(R′)—. In some embodiments, each methylene unit is independently replaced with —N(R′)—, —C(R′)₂— or —C(O)—. In some embodiments, L^{P2} is or comprises an amino acid residue. In some embodiments, L^{P2} is or comprises a peptide.

In some embodiments, L^{P2} is or comprises —[X]pX⁴[X]p′—, wherein each of p, p′, X and X⁴ is independently as described herein. In some embodiments, L^{P2} is or comprises —[X]pX³X⁴[X]p′—, wherein each X and X¹ is independently an amino acid residue, and each of p and p′ is independently 0-10. In some embodiments, L^{P2} is or comprises —X³X⁴—, wherein each X³ and X⁴ is independently as described herein, and X⁴ is bonded to L^{AA2}.

In some embodiments, L^{P2} comprises a —C(R′)₂— group, wherein one of the R′ groups is a second R′ group and the other is a third of the four. In some embodiments, such a —C(R′)₂— group is of an amino acid residue. In some embodiments, such a —C(R′)₂— group is of X⁴. In some embodiments, such a carbon atom is an alpha carbon of an amino acid residue. In some embodiments, such a carbon atom is an alpha carbon of X⁴.

### L^{AA2}

In some embodiments, L^{AA2} is amino acid residue. In some embodiments, L^{AA2} is an amino acid residue that comprises a side chain comprising an acidic or polar group. In some embodiments, L^{AA2} is an amino acid residue that comprises a side chain comprising an acidic group.

In some embodiments, L^{AA2} is L^{AR}, wherein a methylene unit is replaced with —C(R′)(R^{AS})—, wherein each variable is independently as described herein. In some embodiments, L^{A2} is an optionally substituted, bivalent C₁-C₆ (e.g., C₁, C₂, C₃, C₄, C₅, or C₆) aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA2} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA2} is —N(R′)—C(R′)(R^{AS})C(O)—, wherein each variable is independently as described herein. In some embodiments, L^{AA2} is —NH—C(R′)(R^{AS})—C(O)—, wherein each variable is independently as described herein.

In some embodiments, L^{AS2} is L^{AS} as described herein. In some embodiments, R^{AA2} is —CO₂R, wherein R is as described herein. In some embodiments, R is H. In some embodiments, L^{AA2} is a residue of an acidic amino acid residue, e.g., Asp, Glu, etc. In some embodiments, L^{AA2} is X⁵ as described herein.

### L^{P3}

In some embodiments, L^{P3} is a covalent bond. In some embodiments, L^{P3} is an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the length of L^{P3} is 2-10 atoms. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms. In some embodiments, one or more methylene units are independently replaced with —N(R′)—, —C(R′)₂—, —C(O)— or —C(O)N(R′)—. In some embodiments, a methylene unit is replace with —N(R′)—. In some embodiments, a methylene unit is replace with —C(R′)₂—. In some embodiments, a methylene unit is replace with —C(O)—. In some embodiments, a methylene unit is replace with —C(O)N(R′)—. In some embodiments, each methylene unit is independently replaced with —N(R′)—, —C(R′)₂— or —C(O)—. In some embodiments, L^{P3} is or comprises an amino acid residue. In some embodiments, L^{P3} is or comprises a peptide.

### L^{AA3}

In some embodiments, L^{AA3} is amino acid residue. In some embodiments, L^{AA3} is an amino acid residue that comprises a side chain comprising an acidic or polar group. In some embodiments, L^{AA3} is an amino acid residue that comprises a side chain comprising an acidic group.

In some embodiments, L^{AA3} is L^{AR}, wherein a methylene unit is replaced with —C(R′)(R^{AS})—, wherein each variable is independently as described herein. In some embodiments, L^{A3} is an optionally substituted, bivalent C₁-C₆ (e.g., C₁, C₂, C₃, C₄, C₅, or C₆) aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA3} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA3} is —N(R′)—C(R′)(R^{AS})—C(O)—, wherein each variable is independently as described herein. In some embodiments, L^{AA3} is —NH—C(R′)(R^{AS})—C(O)—, wherein each variable is independently as described herein.

In some embodiments, L^{AS3} is L^{AS} as described herein. In some embodiments, R^{AA3} is —CO₂R, wherein R is as described herein. In some embodiments, R is H. In some embodiments, L^{AA3} is a residue of an acidic amino acid residue, e.g., Asp, Glu, etc. In some embodiments, L^{AA3} is X⁶ as described herein.

### L^{P4}

In some embodiments, L^{P4} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the length of L^{P4} is 2-10 atoms. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms. In some embodiments, one or more methylene units are independently replaced with —N(R′)—, —C(R′)₂—, —C(O)— or —C(O)N(R′)—. In some embodiments, a methylene unit is replace with —N(R′)—. In some embodiments, a methylene unit is replace with —C(R′)₂—. In some embodiments, a methylene unit is replace with —C(O)—. In some embodiments, a methylene unit is replace with —C(O)N(R′)—. In some embodiments, each methylene unit is independently replaced with —N(R′)—, —C(R′)₂— or —C(O)—. In some embodiments, L^{P4} is or comprises an amino acid residue. In some embodiments, L^{P4} is or comprises a peptide.

In some embodiments, L^{P4} is or comprises —[X]pX⁷X⁸[X]p′—, wherein each X and X¹¹ is independently an amino acid residue, and each of p and p′ is independently 0-10. In some embodiments, L^{P4} is or comprises —X⁷X⁸—, wherein each X⁷ and X⁸ is independently as described herein, and X⁸ is bonded to L^{AA4}.

### L^{A4}4

In some embodiments, L^{AA4} is amino acid residue. In some embodiments, L^{AA4} is an amino acid residue that comprises a side chain comprising an aromatic group.

In some embodiments, L^{AA4} is L^{AR}, wherein a methylene unit is replaced with —C(R′)(R^{AS})—, wherein each variable is independently as described herein. In some embodiments, L^{AA4} is an optionally substituted, bivalent C₁-C₆ (e.g., C₁, C₂, C₃, C₄, C₅, or C₆) aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{A4} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA4} is —N(R′)—C(R′)(R^{AS})—C(O)—, wherein each variable is independently as described herein. In some embodiments, L^{AA4} is —NH—C(R′)(R^{AS})—C(O)—, wherein each variable is independently as described herein.

In some embodiments, L^{AS4} is L^{AS} as described herein. In some embodiments, R^{AA4} is optionally substituted C₆₋₁₄ aryl. In some embodiments, R^{AAA} is optionally substituted phenyl. In some embodiments, R^{AA4} is phenyl. In some embodiments, R^{AAA4} is optionally substituted 10-membered C₁₀ bicyclic aryl. In some embodiments, R^{AA4} is optionally substituted 5-membered monocyclic heteroaryl having 1-4 heteroatoms. In some embodiments, R^{AA4} is optionally substituted 6-membered monocyclic heteroaryl having 1-4 heteroatoms. In some embodiments, R^{AA4} is optionally substituted 9-membered bicyclic heteroaryl having 1-4 heteroatoms. In some embodiments, R^{AA4} is optionally substituted 10-membered bicyclic heteroaryl having 1-4 heteroatoms. In some embodiments, a heteroaryl has no more than one heteroatom. In some embodiments, a heteroaryl has two or more heteroatoms. In some embodiments, a heteroatom is oxygen. In some embodiments, a heteroatom is nitrogen. In some embodiments, a heteroatom is sulfur. In some embodiments, R^{AA4} is optionally substituted

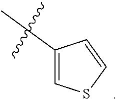 [SMILES: *c1ccsc1]

In some embodiments, R^{AA4} is optionally substituted

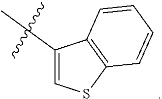 [SMILES: *c1csc2ccccc12]

In some embodiments, R^{AA4} is optionally substituted

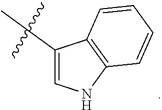 [SMILES: *c1c[nH]c2ccccc12]

In some embodiments, R^{AA4} is an aromatic amino acid residue as described herein. In some embodiments, R^{AA4} is X⁹ as described herein.

### L^{P5}

In some embodiments, L^{P5} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the length of L^{P5} is 2-10 atoms. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms. In some embodiments, one or more methylene units are independently replaced with —N(R′)—, —C(R′)₂—, —C(O)— or —C(O)N(R′)—. In some embodiments, a methylene unit is replace with —N(R′)—. In some embodiments, a methylene unit is replace with —C(R′)₂—. In some embodiments, a methylene unit is replace with —C(O)—. In some embodiments, a methylene unit is replace with —C(O)N(R′)—. In some embodiments, each methylene unit is independently replaced with —N(R′)—, —C(R′)₂— or —C(O)—. In some embodiments, L^{P5} is or comprises an amino acid residue. In some embodiments, L^{P5} is or comprises a peptide.

In some embodiments, L^{P5} is or comprises —[X]pX¹¹[X]p′-, wherein each variable is independently as described herein. In some embodiments, L^{P5} is or comprises —X¹⁰X¹¹—, wherein each X¹⁰ and X¹¹ is independently as described herein, and X¹¹ is bonded to L^{AA5}.

In some embodiments, L^{P5} comprises a —C(R′)₂— group, wherein one of the R′ groups is a fourth R′ group of the four. In some embodiments, such a —C(R′)₂— group is of an amino acid residue. In some embodiments, such a —C(R′)₂— group is of X¹¹. In some embodiments, such a carbon atom is an alpha carbon of an amino acid residue. In some embodiments, such a carbon atom is an alpha carbon of X¹¹

### L^{AA5}

In some embodiments, L^{AA5} is amino acid residue. In some embodiments, L^{A5} is an amino acid residue that comprises a side chain comprising an aromatic group.

In some embodiments, L^{AA5} is L^{AR}, wherein a methylene unit is replaced with —C(R′)(R^{AS})—, wherein each variable is independently as described herein. In some embodiments, L^{A5} is an optionally substituted, bivalent C₁-C₆ (e.g., C₁, C₂, C₃, C₄, C₅, or C₆) aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{A5} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA5} is —N(R′)—C(R′)(R^{AS})C(O)—, wherein each variable is independently as described herein. In some embodiments, L^{AA5} is —NH—C(R′)(R^{AS})—C(O)—, wherein each variable is independently as described herein.

In some embodiments, L^{AS5} is L^{AS} as described herein. In some embodiments, R^{AA5} is optionally substituted C₆₋₁₄ aryl. In some embodiments, R^{AA5} is optionally substituted phenyl. In some embodiments, R^{AA5} is phenyl. In some embodiments, R^{AA5} is optionally substituted 10-membered C₁₀ bicyclic aryl. In some embodiments, R^{AA5} is optionally substituted 5-membered monocyclic heteroaryl having 1-4 heteroatoms. In some embodiments, R^{AA5} is optionally substituted 6-membered monocyclic heteroaryl having 1-4 heteroatoms. In some embodiments, R^{AA5} is optionally substituted 9-membered bicyclic heteroaryl having 1-4 heteroatoms. In some embodiments, R^{AA5} is optionally substituted 10-membered bicyclic heteroaryl having 1-4 heteroatoms. In some embodiments, a heteroaryl has no more than one heteroatom. In some embodiments, a heteroaryl has two or more heteroatoms. In some embodiments, a heteroatom is oxygen. In some embodiments, a heteroatom is nitrogen. In some embodiments, a heteroatom is sulfur. In some embodiments, R^{AA5} is optionally substituted

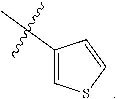 [SMILES: *c1ccsc1]

In some embodiments, R^{AA5} is optionally substituted

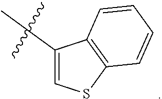 [SMILES: *c1csc2ccccc12]

In some embodiments, R^{AA5} is optionally substituted

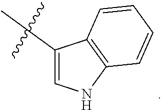 [SMILES: *c1c[nH]c2ccccc12]

In some embodiments, R^{AA5} is an aromatic amino acid residue as described herein. In some embodiments, R^{AA5} is X¹² as described herein.

### L^{P6}

In some embodiments, L^{P6} is a covalent bond. In some embodiments, L^{P6} is an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the length of L^{P6} is 2-10 atoms. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms. In some embodiments, one or more methylene units are independently replaced with —N(R′)—, —C(R′)₂—, —C(O)— or —C(O)N(R′)—. In some embodiments, a methylene unit is replace with —N(R′)—. In some embodiments, a methylene unit is replace with —C(R′)₂—. In some embodiments, a methylene unit is replace with —C(O)—. In some embodiments, a methylene unit is replace with —C(O)N(R′)—. In some embodiments, each methylene unit is independently replaced with —N(R′)—, —C(R′)₂— or —C(O)—. In some embodiments, L^{P6} is or comprises an amino acid residue. In some embodiments, L^{P6} is or comprises a peptide.

### L^{AA6}

In some embodiments, L^{AA6} is amino acid residue. In some embodiments, L^{AA6} is an amino acid residue that comprises a side chain comprising an aromatic group.

In some embodiments, L^{AA6} is L^{AR}, wherein a methylene unit is replaced with —C(R′)(R^{AS})—, wherein each variable is independently as described herein. In some embodiments, L^{AA6} is an optionally substituted, bivalent C₁-C₆ (e.g., C₁, C₂, C₃, C₄, C₅, or C₆) aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA6} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA6} is —N(R′)—C(R′)(R^{AS})—C(O)—, wherein each variable is independently as described herein. In some embodiments, L^{AA6} is —NH—C(R′)(R^{AS})—C(O)—, wherein each variable is independently as described herein.

In some embodiments, L^{AS6} is L^{AS} as described herein. In some embodiments, R^{AA6} is optionally substituted C₆₋₁₄ aryl. In some embodiments, R^{AA6} is optionally substituted phenyl. In some embodiments, R^{AA6} is phenyl. In some embodiments, R^{AA6} is optionally substituted 10-membered C₁₀ bicyclic aryl. In some embodiments, R^{AA6} is optionally substituted 5-membered monocyclic heteroaryl having 1-4 heteroatoms. In some embodiments, R^{AA6} is optionally substituted 6-membered monocyclic heteroaryl having 1-4 heteroatoms. In some embodiments, R^{AA6} is optionally substituted 9-membered bicyclic heteroaryl having 1-4 heteroatoms. In some embodiments, R^{AA6} is optionally substituted 10-membered bicyclic heteroaryl having 1-4 heteroatoms. In some embodiments, a heteroaryl has no more than one heteroatom. In some embodiments, a heteroaryl has two or more heteroatoms. In some embodiments, a heteroatom is oxygen. In some embodiments, a heteroatom is nitrogen. In some embodiments, a heteroatom is sulfur. In some embodiments, R^{AA6} is optionally substituted

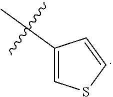 [SMILES: *c1ccsc1]

In some embodiments, R^{AA6} is optionally substituted

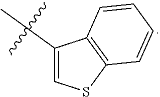 [SMILES: *c1csc2ccccc12]

In some embodiments, R^{AA6} is optionally substituted

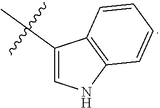 [SMILES: *c1c[nH]c2ccccc12]

In some embodiments, R^{AA6} is an aromatic amino acid residue as described herein. In some embodiments, R^{AA6} is X¹³ as described herein.

### L^{P7}

In some embodiments, L^{P7} is a covalent bond. In some embodiments, L^{P7} is an optionally substituted, bivalent C₁-C₂₅ (e.g., C₁₋₂₀, C₁₋₁₅, C₁₋₁₀, C₁₋₅, C₁, C₂, C₃, C₄, C₅, C₆, C₇, C₈, C₉, C₁₀, C₁₁, C₁₂, C₁₃, C₁₄, C₁₅, C₁₆, C₁₇, C18, C₁₉, or C₂₀) aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{P7} is an optionally substituted, bivalent C₁-C₂₅ (e.g., C₁₋₂₀, C₁₋₁₅, C₁₋₁₀, C₁₋₅, C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) aliphatic group, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{P7} is an optionally substituted, bivalent C₁-C₂₀ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{P7} is an optionally substituted, bivalent C₁-C₁₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{P7} is an optionally substituted, bivalent C₁-C₁₀ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

### L^{AS}

In some embodiments, L^{AS} is a covalent bond. In some embodiments, L^{AS} is an optionally substituted, bivalent C₁-C₁₀ (e.g., C₁₋₅, C₁, C₂, C₃, C₄, C₅, C₆, C₇, C₈, C₉, or C₁₀) aliphatic group, wherein one or more methylene units of the group optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{AS} is an optionally substituted, bivalent C₁-C₁₀ aliphatic group, wherein one or more methylene units of the group optionally and independently replaced with —C(R′)₂—, -Cy-, —O—, —S—, —N(R′)—, —C(O)—, —S(O)—, or —S(O)₂—. In some embodiments, L^{AS} is an optionally substituted, bivalent C₁-C₁₀ aliphatic group, wherein one or more methylene units of the group optionally and independently replaced with —O—, —S—, or —N(R′)—. In some embodiments, L^{AS} is an optionally substituted, bivalent C₁-C₁₀ alkylene group. In some embodiments, L^{AS} is optionally substituted —CH₂—. In some embodiments, L^{AS} is —CH₂—. In some embodiments, the length of L^{AS} is 1, 2, 3, 4, 5, 6, 7, 8, 9, or 10 atoms. In some embodiments, it is 1 atom. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms.

In some embodiments, an agent of formula I is a stapled peptide as described herein. In some embodiments, an agent of formula I is an agent selected from Table E2 or a pharmaceutically acceptable salt thereof.

Among other things, the present disclosure provides agents, e.g. peptides, that can bind to beta-catenin. In some embodiments, a peptide is a stapled peptide. In some embodiments, a peptide is a stitched peptide. In some embodiments, an agent binds to a TCF site of beta-catenin. In some embodiments, an agent competes with TCF for beta-catenin binding.

In some embodiments, the present disclosure provides an agent, which is or comprises a peptide comprising:

X¹X²X³X⁴X⁵X⁶X⁷X⁸X⁹X¹⁰X¹¹X¹²x¹³,

wherein:
each of p14, p15, p16 and p17 is independently 0 or 1;
   each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein the agent binds to beta-catenin.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein the agent binds to beta-catenin. In some embodiments, X² comprises a side chain comprising an acidic or polar group. In some embodiments, X² comprises a side chain comprising an acidic. In some embodiments, X⁵ comprises a side chain comprising an acidic or polar group. In some embodiments, X⁵ comprises a side chain comprising an acidic. In some embodiments, X⁹ comprises a side chain comprising an optionally substituted aromatic group. In some embodiments, X¹² comprises a side chain comprising an optionally substituted aromatic group. In some embodiments, X¹³ comprises a side chain comprising an optionally substituted aromatic group. In some embodiments, X¹ and X⁴ are independently amino acid residues suitable for stapling. In some embodiments, X⁴ and X¹¹ are independently amino acid residues suitable for stapling. In some embodiments, X¹⁰ and X¹⁴ are independently amino acid residues suitable for stapling. In some embodiments, X⁷ and X¹⁴ are independently amino acid residues suitable for stapling. In some embodiments, X¹ and X⁴ are independently amino acid residues suitable for stapling, and X⁴ and X¹¹ are independently amino acid residues suitable for stapling. In some embodiments, X¹ and X⁴ are independently amino acid residues suitable for stapling, and X¹⁰ and X¹⁴ are independently amino acid residues suitable for stapling. In some embodiments, X¹ and X⁴ are independently amino acid residues suitable for stapling, and X⁷ and X¹⁴ are independently amino acid residues suitable for stapling. In some embodiments, X³ and X¹⁰ are independently amino acid residues suitable for stapling. In some embodiments, X¹ and X⁴ are stapled. In some embodiments, X⁴ and X¹¹ are stapled. In some embodiments, X¹⁰ and X¹⁴ are stapled. In some embodiments, X⁷ and X¹⁴ are stapled. In some embodiments, X³ and X¹⁰ are stapled. In some embodiments, X¹ and X⁴ are stapled, and X⁴ and X¹¹ are stapled. In some embodiments, X¹ and X⁴ are stapled, and X¹⁰ and X¹⁴ are stapled. In some embodiments, X¹ and X⁴ are stapled, and X⁷ and X¹⁴ are stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein the agent binds to beta-catenin. In some embodiments, X² comprises a side chain comprising an acidic or polar group. In some embodiments, X² comprises a side chain comprising an acidic. In some embodiments, X⁵ comprises a side chain comprising an acidic or polar group. In some embodiments, X⁵ comprises a side chain comprising an acidic. In some embodiments, X⁹ comprises a side chain comprising an optionally substituted aromatic group. In some embodiments, X¹² comprises a side chain comprising an optionally substituted aromatic group. In some embodiments, X¹³ comprises a side chain comprising an optionally substituted aromatic group. In some embodiments, X¹ and X⁴ are independently amino acid residues suitable for stapling. In some embodiments, X⁴ and X¹¹ are independently amino acid residues suitable for stapling. In some embodiments, X¹ and X⁴ are independently amino acid residues suitable for stapling, and X⁴ and X¹¹ are independently amino acid residues suitable for stapling. In some embodiments, X³ and X¹⁰ are independently amino acid residues suitable for stapling. In some embodiments, X¹ and X⁴ are stapled. In some embodiments, X⁴ and X¹¹ are stapled. In some embodiments, X¹ and X⁴ are stapled, and X⁴ and X¹¹ are stapled. In some embodiments, X³ and X¹⁰ are stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:

X¹ and X⁴, and/or X⁴ and X¹¹ are independently amino acid residues suitable for stapling, or are stapled, X³ and X¹⁰ are independently amino acid residues suitable for stapling, or are stapled, X¹ and X⁴, and/or X¹⁰ and X¹⁴ are independently amino acid residues suitable for stapling, or are stapled, or X¹ and X⁴, and/or X⁷ and X¹⁴ are independently amino acid residues suitable for stapling, or are stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and/or X⁴ and X¹¹ are independently amino acid residues suitable for stapling, or are stapled, or X³ and X¹⁰ are independently amino acid residues suitable for stapling, or are stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and/or X⁴ and X¹¹ are independently amino acid residues suitable for stapling, or X³ and X¹⁰ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and X⁴ and X¹¹ are independently amino acid residues suitable for stapling, or X³ and X¹⁰ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and X⁴ and X¹¹ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic group;
X⁵ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and X⁴ and X¹¹ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X³ and X¹⁰ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic group;
X⁵ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X³ and X¹⁰ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X⁴ and X¹¹ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic group;
X⁵ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X⁴ and X¹¹ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and/or X⁴ and X¹¹ are independently stapled, or X³ and X¹⁰ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and X⁴ and X¹¹ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic group;
X⁵ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and/or X⁴ and X¹¹ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X³ and X¹⁰ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic group;
X⁵ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X³ and X¹⁰ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X⁴ and X¹¹ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,

wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic group;
X⁵ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X⁴ and X¹¹ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein the agent binds to beta-catenin. In some embodiments, X² comprises a side chain comprising an acidic or polar group. In some embodiments, X² comprises a side chain comprising an acidic. In some embodiments, X⁵ comprises a side chain comprising an acidic or polar group. In some embodiments, X⁵ comprises a side chain comprising an acidic. In some embodiments, X⁹ comprises a side chain comprising an optionally substituted aromatic group. In some embodiments, X¹² comprises a side chain comprising an optionally substituted aromatic group. In some embodiments, X¹³ comprises a side chain comprising an optionally substituted aromatic group. In some embodiments, X¹ and X⁴ are independently amino acid residues suitable for stapling. In some embodiments, X⁴ and X¹¹ are independently amino acid residues suitable for stapling. In some embodiments, X¹⁰ and X¹⁴ are independently amino acid residues suitable for stapling. In some embodiments, X⁷ and X¹⁴ are independently amino acid residues suitable for stapling. In some embodiments, X¹ and X⁴ are independently amino acid residues suitable for stapling, and X⁴ and X¹¹ are independently amino acid residues suitable for stapling. In some embodiments, X¹ and X⁴ are independently amino acid residues suitable for stapling, and X¹⁰ and X¹⁴ are independently amino acid residues suitable for stapling. In some embodiments, X¹ and X⁴ are independently amino acid residues suitable for stapling, and X⁷ and X¹⁴ are independently amino acid residues suitable for stapling. In some embodiments, X³ and X¹⁰ are independently amino acid residues suitable for stapling. In some embodiments, X¹ and X⁴ are stapled. In some embodiments, X⁴ and X¹¹ are stapled. In some embodiments, X¹⁰ and X¹⁴ are stapled. In some embodiments, X⁷ and X¹⁴ are stapled. In some embodiments, X³ and X¹⁰ are stapled. In some embodiments, X¹ and X⁴ are stapled, and X⁴ and X¹¹ are stapled. In some embodiments, X¹ and X⁴ are stapled, and X¹⁰ and X¹⁴ are stapled. In some embodiments, X¹ and X⁴ are stapled, and X⁷ and X¹⁴ are stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein the agent binds to beta-catenin. In some embodiments, X² comprises a side chain comprising an acidic or polar group. In some embodiments, X² comprises a side chain comprising an acidic. In some embodiments, X⁵ comprises a side chain comprising an acidic or polar group. In some embodiments, X⁵ comprises a side chain comprising an acidic. In some embodiments, X⁹ comprises a side chain comprising an optionally substituted aromatic group. In some embodiments, X¹² comprises a side chain comprising an optionally substituted aromatic group. In some embodiments, X¹³ comprises a side chain comprising an optionally substituted aromatic group. In some embodiments, X¹ and X⁴ are independently amino acid residues suitable for stapling. In some embodiments, X⁴ and X¹¹ are independently amino acid residues suitable for stapling. In some embodiments, X¹ and X⁴ are independently amino acid residues suitable for stapling, and X⁴ and X¹¹ are independently amino acid residues suitable for stapling. In some embodiments, X³ and X¹⁰ are independently amino acid residues suitable for stapling. In some embodiments, X¹ and X⁴ are stapled. In some embodiments, X⁴ and X¹¹ are stapled. In some embodiments, X¹ and X⁴ are stapled, and X⁴ and X¹¹ are stapled. In some embodiments, X³ and X¹⁰ are stapled.

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹,
X²², and X²³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and/or X⁴ and X¹¹ are independently amino acid residues suitable for stapling, or are stapled, X³ and X¹⁰ are independently amino acid residues suitable for stapling, or are stapled, X¹ and X⁴, and/or X¹⁰ and X¹⁴ are independently amino acid residues suitable for stapling, or are stapled, or X¹ and X⁴, and/or X⁷ and X¹⁴ are independently amino acid residues suitable for stapling, or are stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and/or X⁴ and X¹¹ are independently amino acid residues suitable for stapling, or are stapled, or X³ and X¹⁰ are independently amino acid residues suitable for stapling, or are stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and/or X⁴ and X¹¹ are independently amino acid residues suitable for stapling, or X³ and X¹⁰ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and X⁴ and X¹¹ are independently amino acid residues suitable for stapling, or X³ and X¹⁰ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and X⁴ and X¹¹ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic group;
X⁵ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and X⁴ and X¹¹ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X³ and X¹⁰ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic group;
X⁵ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X³ and X¹⁰ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X⁴ and X¹¹ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic group;
X⁵ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X⁴ and X¹¹ are independently amino acid residues suitable for stapling.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and/or X⁴ and X¹¹ are independently stapled, or X³ and X¹⁰ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and X⁴ and X¹¹ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic group;
X⁵ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and/or X⁴ and X¹¹ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X³ and X¹⁰ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic group;
X⁵ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X³ and X¹⁰ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X⁴ and X¹¹ are independently stapled.

In some embodiments, the present disclosure provides an agent, which is or comprises a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,

wherein:
each of p14, p15, p16, p17, p18, p19, p20, p21, p22, and p23 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue, wherein:
X² comprises a side chain comprising an acidic group;
X⁵ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X⁴ and X¹¹ are independently stapled.

Various types of amino acid residues can be used for X¹, e.g., a residue of an amino acid of formula A-I, A-II, A-III, A-IV, A-V, A-VI, etc. or a salt thereof in accordance with the present disclosure. In some embodiments, X¹ is —N(R^{a1})-L^{a1}-C(R^{a2})(R^{a3})-L^{a2}-C(O)—, wherein each variable is independently as described herein. In some embodiments, X¹ is —N(R^{a1})—C(R^{a2})(R^{a3})—C(O)—, wherein each variable is independently as described herein. In some embodiments, X¹ is —N(R^{a1})—C(R^{a2})H—C(O)—, wherein each variable is independently as described herein. In some embodiments, R^{a1} is —H. In some embodiments, R^{a3} is —H.

As shown herein (e.g., for various amino acids and residues thereof), in various embodiments, L^{a} is L as described herein. For example, in some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is —(CH₂)ₙ—, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, one or more methylene units of L are independently replaced with —C(O)—, —N(R′)—, -Cy- or —O—. In some embodiments, a methylene unit is replaced with —C(O)—. In some embodiments, a methylene unit is replaced with —N(R′)—. In some embodiments, a methylene unit is replaced with -Cy-. In some embodiments, -Cy- is optionally substituted phenylene. In some embodiments, -Cy- is 1,2-phenylene. In some embodiments, a methylene unit is replaced with —O—. In some embodiments, L is —C(O)—(CH₂)ₙ—. In some embodiments, L is —C(O)—(CH₂)₂—. In some embodiments, L is —C(O)—(CH₂)₃—. In some embodiments, L is —C(O)—1,2-phenylene-O—CH₂—. As appreciated by those skilled in the art, embodiments described for each group or moiety, e.g., L, is applicable to all groups that can be such a group or moiety (e.g., La, L^{s1}, L^{s2}, L^{s3}, etc.), no matter where such embodiments are described.

In some embodiments, X¹ a residue of amino acid that comprises an optionally substituted ring. In some embodiments, the amino group of X¹ is part of an optionally substituted ring. In some embodiments, X¹ is an amino acid as described herein (e.g., of formula A-I, A-II, A-III, etc.), wherein R^{a1} and R^{a3} are taken together to form an optionally substituted ring, e.g., an optionally substituted 3-10 membered ring. In some embodiments, R^{a1} and R^{a3} are taken together with their intervening atoms to form an optionally substituted 3-10 membered saturated or partially saturated ring having, in addition to the intervening atoms, 0-5 heteroatoms. In some embodiments, a formed ring is saturated. In some embodiments, a formed ring is monocyclic. In some embodiments, a formed ring has no heteroatoms in addition to the intervening atoms. In some embodiments, L^{a1} and L^{a2} are covalent bond. In some embodiments, a formed ring is unsubstituted. In some embodiments, a formed ring is substituted. In some embodiments, a substitute comprises a double bond which is suitable for metathesis with another double bond to form a staple. In some embodiments, X¹ is Pro. In some embodiments, X¹ is alphaMePro (methyl replacing —H at alpha carbon). In some embodiments, X¹ comprises a hydrophobic side chain. In some embodiments, side chain of X¹ comprises an optionally substituted aromatic ring. In some embodiments, X¹ is Phe. In some embodiments, X¹ is Ala. In some embodiments, none of R^{a2} and R^{a3} are hydrogen. In some embodiments, X¹ is Aib. In some embodiments, X¹ is comprises a side chain which comprises an acidic group, e.g., —COOH. In some embodiments, X¹ is Asp. In some embodiments, X¹ is an amino acid reside suitable for stapling. In some embodiments, X¹ comprises a double bond, e.g., a terminal double bond in its side chain. In some embodiments, X¹ is PL3.

In some embodiments, X¹ is an amino acid reside suitable for stapling.

In some embodiments, an amino acid residue suitable for stapling comprises a double bond, e.g., a terminal double bond in its side chain. In some embodiments, it has a side chain having the structure of -L^{a}-CH═CH₂. In some embodiments, it is a residue of an amino acid having the structure of formula A-II or A-III or a salt thereof. In some embodiments, X¹ is —N(R^{a1})-L^{a1}-C(-L^{a}-CH═CH₂)(R^{a3})-L^{a2}-C(O)—, wherein each variable is independently as described herein. In some embodiments, X¹ is —N(R^{a1})—C(-L^{a}-CH═CH₂)(R^{a3})—C(O)—, wherein each variable is independently as described herein. In some embodiments, X¹ is a residue of PL3 and stapled.

In some embodiments, X¹ is or comprises a residue of an amino acid or a moiety selected from Table A-I, Table A-II, Table A-III and Table A-IV.

In some embodiments, X¹ is a residue of Bn3OAllyl, BzAm3Oallyl, HypBzEs3OAllyl, HypEs4, HypEs5, HypPAc3OAllyl, MePro, NMebAla, PAc3OAllyl, ProAm5, ProAm6, ProBzAm3OAllyl, ProPAc3OAllyl, ProSAm3, PyrR, Sar, Aib, Ala, Asp, Gly, Phe, PL3, Pro, R3, or R5.

In some embodiments, X¹ is a residue of Bn3OAllyl, BzAm3Oallyl, HypBzEs3OAllyl, HypEs4, HypEs5, HypPAc3OAllyl, MePro, NMebAla, PAc3OAllyl, ProAm5, ProAm6, ProBzAm3OAllyl, ProPAc3OAllyl, ProSAm3, PyrR, or Sar.

In some embodiments, X¹ is a residue of Aib, Ala, Asp, Gly, Phe, PL3, Pro, R3, or R5.

In some embodiments, X¹ is stapled (a staple bonds to X¹). In some embodiments, X¹ is PL3 and stapled. In some embodiments, X¹ is stapled with X⁴. In some embodiments, a staple connecting a pair of amino acid residues, e.g., X¹ and X⁴, has the structure of L^{s}, -L^{s1}-L^{s2}-L^{s3}-, wherein L^{s1} is L^{a} of one amino acid residue, e.g., X¹, and L^{s3} is L^{a} of the other amino acid residue, e.g., X⁴.

As described herein, in some embodiments, a staple is L^{s}. In some embodiments, L^{s1} is L^{a} of one amino acid residue of a pair of stapled amino acid residues, and L^{s3} is L^{a} of the other amino acid residue of a pair of stapled amino acid residues. In some embodiments, L^{s} is -L^{a}-L^{s2}-L^{a}-, wherein each variable is independently as described herein. Various embodiments of L^{a} are described herein. In some embodiments, L^{s1} is an optionally substituted bivalent linear or branched C₁₋₁₀ hydrocarbon chain. In some embodiments, L^{s3} is an optionally substituted bivalent linear or branched C₁₋₁₀ hydrocarbon chain. In some embodiments, each of L^{s1} and L^{s3} is independently an optionally substituted bivalent linear or branched C₁₋₁₀ hydrocarbon chain. In some embodiments, each of L^{s1} and L^{s3} is independently —(CH₂)ₙ—, wherein n is 1-10. In some embodiments, L^{s1} is —CH₂—. In some embodiments, L^{s3} is —(CH₂)₃—.

In some embodiments, L^{s2} is L as described herein. In some embodiments, L is optionally substituted —CH═CH—. In some embodiments, L is optionally substituted —CH₂—CH₂—. In some embodiments, L is —CH₂—CH₂—.

In some embodiments, L^{s} is —CH₂—CH═CH—(CH₂)₃—. In some embodiments, L^{s} is —(CH₂)₆—. In some embodiments, such a staple connects X¹ and X⁴. In some embodiments, such a staple may connect other pairs of stapled amino acid residues.

In some embodiments, a staple, e.g., L^{s}, is bonded to two backbone atoms. In some embodiments, it is bonded to two carbon backbone atoms. In some embodiments, it is independently bonded to an alpha carbon atom of an amino acid residue at each end.

Various types of amino acid residues can be used for X², e.g., a residue of an amino acid of formula A-I, A-II, A-III, A-IV, A-V, A-VI, etc. or a salt thereof in accordance with the present disclosure. In some embodiments, X² is —N(R^{a1})-L^{a1}-C(R^{a2})(R^{a3})-L^{a2}-C(O)—, wherein each variable is independently as described herein. In some embodiments, X² is —N(R^{a1})—C(R^{a2})(R^{a3})—C(O)—, wherein each variable is independently as described herein. In some embodiments, X² is —N(R^{a1})—C(R^{a2})H—C(O)—, wherein each variable is independently as described herein. In some embodiments, R^{a1} is —H. In some embodiments, R^{a3} is —H.

In some embodiments, X² is a residue of amino acid that comprises an acidic or polar group. In some embodiments, X² is a residue of amino acid whose side chain comprises an acidic group, e.g., a —COOH group or a salt form thereof (e.g., a compound of formula A-IV, etc.) (in some embodiments, may be referred to as an “acidic amino acid residue”).

In some embodiments, an amino acid residue whose side chain comprises an acidic group comprises —COOH in its side chain. In some embodiments, it is a residue of an amino acid having the structure of formula A-IV or a salt thereof. In some embodiments, it is a residue of amino acid having the structure of formula PA, PA-a, PA-b, PA-c, etc. In some embodiments, RPA is —H and R^{PS} and R^{PC} are —OH. In some embodiments, it is —N(R^{a1})-L^{a1}-C(-L^{a}-COOH)(R^{a3})-L^{a2}-C(O)—. In some embodiments, it is —NH-L^{a1}-C(-L^{a}-COOH)(R^{a3})-L^{a2}-C(O)—. In some embodiments, it is —NH—CH(-L^{a}-COOH)—C(O)—.

As described herein, L^{a} is L as described herein. In some embodiments, L is an optionally substituted bivalent linear or branched C₁₋₁₀ hydrocarbon chain wherein one or more methylene units of L are independently replaced with —C(O)—, —N(R′)—, -Cy- or —O—. In some embodiments, L is an optionally substituted bivalent linear C₁₋₁₀ hydrocarbon chain wherein one or more methylene units of L are independently replaced with —C(O)—, —N(R′)—, -Cy- or —O—. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)ₙ— wherein n is 1-10. In some embodiments, L is —(CH₂)ₙ—. In some embodiments, L is —CH₂—. In some embodiments, L is —(CH₂)₂—. In some embodiments, L is —(CH₂)₃—. In some embodiments, L is —(CH₂)₄—.

In some embodiments, X² is a residue of Asp, Glu, RbGlu, SbGlu, NMeD, and isoDAsp. In some embodiments, X² is a residue of Asp, Glu, RbGlu, SbGlu, and isoDAsp. In some embodiments, X² is a residue of Asp. As appreciated by those skilled in the art, at physiological pH (about pH 7.4), an acidic group such as —COOH may exist, in some embodiments, predominantly, as its negatively charged form, e.g., —COO⁻.

In some embodiments, X² is a residue of amino acid (e.g., of formula A-I, A-II, A-III, A-IV, A-V, A-VI, etc. or a salt thereof) whose side chain comprises a polar group (in some embodiments, may be referred to as a “polar amino acid residue”; in some embodiments, it does not include amino acid residue whose side chains are electrically charged at, e.g., about pH 7.4).

In some embodiments, an amino acid residue whose side chain comprises a polar group is —N(R^{a1})-L^{a1}-C(R^{a2})(R^{a3})-L^{a2}-C(O)—. In some embodiments, an amino acid residue whose side chain comprises a polar group is —N(R^{a1})—C(R^{a2})(R^{a3})—C(O)—. In some embodiments, an amino acid residue whose side chain comprises an amide group, e.g., —C(O)N(R′)₂ such as —CONH₂. In some embodiments, R^{a2} is -L^{a}-C(O)N(R′)₂ wherein each variable is independently as described herein. In some embodiments, R^{a2} is -L^{a}-C(O)NH₂ wherein L is independently as described herein. In some embodiments, L^{a} is L′ as described herein. In some embodiments, R^{a3} is H. In some embodiments, such a polar amino acid residue is Asn. In some embodiments, it is MeAsn. In some embodiments, an amino acid residue whose side chain comprises a polar group is an amino acid residue whose side chain comprises —OH. In some embodiments, R^{a2} is -L^{a}-OH wherein each variable is independently as described herein. In some embodiments, R^{a2} is -L^{a}-OH wherein L is independently as described herein. In some embodiments, L^{a} is L′ as described herein. For example, in some embodiments, such an amino acid residue is a residue of Hse, Ser, aThr, or Thr. In some embodiments, it is a residue of Hse, Ser, or aThr. In some embodiments, it is a residue of Hse. In some embodiments, it is a residue of Ser. In some embodiments, it is a residue of aThr. In some embodiments, it is a residue of Thr. Other polar amino acid residues are described herein and can be utilized at various amino acid residue positions.

In some embodiments, X² is a residue of amino acid whose side chain comprises —OH. For example, in some embodiments, X² is a residue of Hse. In some embodiments, X² is a residue of amino acid whose side chain comprises an amide group, e.g., —CONH₂. For example, in some embodiments, X² is a residue of Asn.

In some embodiments, X² comprises a side chain which is hydrophobic, is aliphatic, is aromatic, etc.

In some embodiments, X² is a residue of Asp, Asn, RbGlu, Phe, Glu, Ile, NMeD, Ala, Dab, Gln, His, Hse, isoDAsp, Leu, Ser, tetz, [MeSO2]Dap, [Tf]Dap, 3FF, 3MeF, SbGlu, or Tyr. In some embodiments, X² is selected from Asp, Hse, Asn, Glu, RbGlu, SbGlu, and isoDAsp (as appreciated by those skilled in the art, an amino acid code can refer to an amino acid and/or a residue thereof depending on context).

In some embodiments, X² is or comprises a residue of an amino acid or a moiety selected from Table A-IV.

In some embodiments, X² is a residue of [CH2CMe2CO2H]TriAzDap, [CMe2CO2H]TriAzDap, [Et]AspE, [EtSSEt]AspE, [EtSSHex]AspE, [EtSSPh]AspE, [EtSSpy]AspE, [Me]AspE, AcAsp, AspSH, RbOHAsp, [MeSO2]Dap, [Tf]Dap, 2COOHF, 3COOHF, 3FF, 3MeF, 4Thz, 4Tria, Aad, Ala, Asn, Asp, Dab, Gln, Glu, His, Hse, Ile, isoDAsp, Leu, NMeD, Phe, PL3, R3, R5, RbGlu, SbGlu, Ser, tetz, TfeGA, Thr, or Tyr.

In some embodiments, X² is a residue of [CH2CMe2CO2H]TriAzDap, [CMe2CO2H]TriAzDap, [Et]AspE, [EtSSEt]AspE, [EtSSHex]AspE, [EtSSPh]AspE, [EtSSpy]AspE, [Me]AspE, AcAsp, AspSH, or RbOHAsp.

In some embodiments, X² is a residue of [MeSO2]Dap, [Tf]Dap, 2COOHF, 3COOHF, 3FF, 3MeF, 4Thz, 4Tria, Aad, Ala, Asn, Asp, Dab, Gln, Glu, His, Hse, Ile, isoDAsp, Leu, NMeD, Phe, PL3, R3, R5, RbGlu, SbGlu, Ser, tetz, TfeGA, Thr, or Tyr.

Various types of amino acid residues can be used for X³, e.g., a residue of an amino acid of formula A-I, A-II, A-III, A-IV, A-V, A-VI, etc. or a salt thereof in accordance with the present disclosure. In some embodiments, X³ is —N(R^{a1})-L^{a1}-C(R^{a2})(R^{a3})-L^{a2}-C(O)—, wherein each variable is independently as described herein. In some embodiments, X³ is —N(R^{a1})—C(R^{a2})(R^{a3})—C(O)—, wherein each variable is independently as described herein. In some embodiments, X³ is —N(R^{a1})—C(R^{a2})H—C(O)—, wherein each variable is independently as described herein. In some embodiments, R^{a1} is —H. In some embodiments, R^{a3} is —H.

In some embodiments, L^{a} is L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is —(CH₂)ₙ—, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(O)—, —N(R′)—, -Cy- or —O—. In some embodiments, L is an optionally substituted bivalent linear C₁₋₁₀ hydrocarbon chain wherein one or more methylene units of L are independently replaced with —C(O)—, —N(R′)—, -Cy- or —O—. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is —CH₂—. In some embodiments, L is —CH₂—N(R′)—CH₂—. In some embodiments, R′ is Bn. In some embodiments, R′ is —C(O)R. In some embodiments, R is phenyl. In some embodiments, R is t-butyl. In some embodiments, R is cyclohexyl.

In some embodiments, X³ is a hydrophobic amino acid residue.

In some embodiments, a hydrophobic amino acid residue is an amino acid residue whose side chain is an optionally substituted aliphatic group. In some embodiments, it is a residue of an amino acid whose side chain is optionally substituted C₁₋₁₀ alkyl. In some embodiments, it is a residue of an amino acid whose side chain is C₁₋₁₀ alkyl. In some embodiments, it is a residue of an amino acid whose side chain is C₁₋₁₀ aliphatic optionally substituted with one or more non-polar and non-charged groups. In some embodiments, it is a residue of an amino acid whose side chain is C₁₋₁₀ alkyl optionally substituted with one or more non-polar and non-charged groups. In some embodiments, it is a residue of an amino acid whose side chain is C₁₋₁₀ aliphatic optionally substituted with one or more hydrophobic substituents. In some embodiments, it is a residue of an amino acid whose side chain is C₁₋₁₀ aliphatic. In some embodiments, it is a residue of an amino acid whose side chain is C₁₋₁₀ alkyl. Various hydrophobic amino acid residues can be utilized in accordance with the present disclosure.

In some embodiments, a hydrophobic amino acid residue, e.g., X³, has the structure of —NH₂—C(R^{a2})(R^{a3})—C(O)— or —NH—C(R^{a2})H—C(O)— wherein each variable is independently as described herein. As described herein, R^{a2} is -L^{a}-R′. In some embodiments, R′ is R as described herein. In some embodiments, R is optionally substituted group selected from C₁₋₁₀ aliphatic, phenyl, 10-membered aryl, and 5-10 membered heteroaryl having 1-5 heteroatoms. In some embodiments, each substituent, if any, is independently a non-polar group. In some embodiments, R is optionally substituted C₁₋₁₀ aliphatic. In some embodiments, R is optionally substituted C₁₋₁₀ alkyl. In some embodiments, R is C₁₋₁₀ aliphatic. In some embodiments, R is C₁₋₁₀ alkyl. For example, in some embodiments, R is methyl. In some embodiments, R is isopropyl. In some embodiments, R is 1-methylpropyl. In some embodiments, R is 2-methylpropyl. In some embodiments, R is optionally substituted aryl. In some embodiments, R is aryl. In some embodiments, R is optionally substituted phenyl. In some embodiments, R is phenyl. In some embodiments, R is optionally substituted 5−6 membered heteroaryl having 1-4 heteroatoms. In some embodiments, R is optionally substituted 5−6 membered heteroaryl having 1 heteroatom. In some embodiments, R is 5−6 membered heteroaryl having 1-4 heteroatoms. In some embodiments, R is 5−6 membered heteroaryl having 1 heteroatom. In some embodiments, R is optionally substituted 9-10 membered heteroaryl having 1-5 heteroatoms. In some embodiments, R is optionally substituted 9-10 membered heteroaryl having 1 heteroatom. In some embodiments, R is 9-10 membered heteroaryl having 1-4 heteroatoms. In some embodiments, R is 9-10 membered heteroaryl having 1 heteroatom. In some embodiments, a heteroatom is nitrogen. In some embodiments, a heteroatom is oxygen. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)ₙ—, wherein n is 1-10. In some embodiments, L is —(CH₂)ₙ—, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(O)—, —N(R′)—, -Cy- or —O—. In some embodiments, L is an optionally substituted bivalent linear C₁₋₁₀ hydrocarbon chain wherein one or more methylene units of L are independently replaced with —C(O)—, —N(R′)—, -Cy- or —O—. In some embodiments, a hydrophobic amino acid residue is a residue of Ala, Val, Ile, Leu, Met, Phe, Tyr, Trp, etc. Other hydrophobic amino acid residues are described herein and can be utilized at various amino acid residue positions.

In some embodiments, X³ comprises a side chain comprising a cycloaliphatic group (e.g., a 4-, 5-, or 6-membered cycloalkyl group).

In some embodiments, X³ comprises a side chain which is or comprises an optionally substituted aromatic group (in some embodiments, may be referred to as an “aromatic amino acid residue”).

In some embodiments, an aromatic amino acid residue has a side chain which is or comprises an optionally substituted aromatic group. In some embodiments, an aromatic amino acid residue, e.g., X³, has the structure of —NH₂—C(R^{a2})(R^{a3})—C(O)— or —NH—C(R^{a2})H—C(O)— wherein each variable is independently as described herein, and R^{a2} comprises an optionally substituted aromatic group.

In some embodiments, an aromatic amino acid residue has a side chain which is or comprises an optionally substituted aromatic group, wherein each substituent of the aromatic group is independently halogen. In some embodiments, it comprises a side chain which is or comprises two optionally substituted aromatic groups. In some embodiments, it comprises a side chain which is or comprises an optionally substituted aromatic group, wherein each substituent of the aromatic group is independently selected from halogen or —OH. In some embodiments, an aromatic group is phenyl. In some embodiments, an aromatic group is optionally substituted 8-10 membered bicyclic aryl or heteroaryl having 0-5 heteroatoms. In some embodiments, an aromatic group is optionally substituted 9-10 membered bicyclic aryl or heteroaryl having one heteroatom. In some embodiments, it is a residue of an amino acid of formula A-I or a salt thereof. In some embodiments, an amino acid residue has the structure of —NH—C(R^{a2})(R^{a3})—C(O)— or —NH—CH(R^{a3})—C)O)—. As described herein, R^{a3} is -L^{a}-R′ wherein each variable is independently as described herein. In some embodiments, R′ is an optionally substituted group selected from phenyl, 10-membered bicyclic aryl, 5−6 membered heteroaryl having 1-4 heteroatoms, and 9-10 membered bicyclic heteroaryl having 1-5 heteroatoms. In some embodiments, each substituent is independently halogen or —OH. In some embodiments, R′ is optionally substituted phenyl. In some embodiments, R′ is phenyl. In some embodiments, R′ is optionally substituted aryl. In some embodiments, R′ is aryl. In some embodiments, R′ is optionally substituted 5-membered heteroaryl having 1-4 heteroatoms. In some embodiments, R′ is optionally substituted 5-membered heteroaryl having 1 heteroatom. In some embodiments, R′ is 5−6 membered heteroaryl having 1-4 heteroatoms. In some embodiments, R′ is 5−6 membered heteroaryl having 1 heteroatom. In some embodiments, R′ is optionally substituted 9-10 membered heteroaryl having 1-5 heteroatoms. In some embodiments, R′ is optionally substituted 9-10 membered heteroaryl having 1 heteroatom. In some embodiments, R′ is 9-10 membered heteroaryl having 1-4 heteroatoms. In some embodiments, R′ is 9-10 membered heteroaryl having 1 heteroatom. In some embodiments, a heteroatom is nitrogen. In some embodiments, a heteroatom is oxygen. In some embodiments, a heteroatom is sulfur. In some embodiments, L^{a} is a covalent bond. In some embodiments, L^{a} is optionally substituted —(CH₂)ₙ— wherein n is 1-10. In some embodiments, L^{a} is —(CH₂)ₙ—. In some embodiments, n is 1. In some embodiments, n is 2. In some embodiments, n is 3. In some embodiments, L^{a} is —CH(Ph)-. In some embodiments, an aromatic amino acid residue is Phe. In some embodiments, an aromatic amino acid residue is Tyr. In some embodiments, an aromatic amino acid residue is Trp. Other aromatic amino acid residues are described herein and can be utilized at various amino acid residue positions.

In some embodiments, X³ is a residue of an amino acid suitable for stapling as described herein. In some embodiments, X³ is —N(R^{a1})-L^{a1}-C(-L^{a}-CH═CH₂)(R^{a3})-L^{a2}-C(O)—, wherein each variable is independently as described herein. In some embodiments, X³ is —N(R^{a1})—C(-L^{a}-CH═CH₂)(R^{a3})—C(O)—, wherein each variable is independently as described herein. In some embodiments, a methylene unit of L^{a} is replaced with —N(R′)— or —N(R′)C(O)O—. In some embodiments, R′ of —N(R′)— or —N(R′)C(O)O— and R^{a3} are taken together with their intervening atoms to form an optionally substituted 3-10 membered ring having 0-5 heteroatoms in addition to the intervening atoms. In some embodiments, there are no heteroatoms in addition to the intervening atoms. In some embodiments, there are no heteroatoms in addition to the nitrogen to which R′ is attached. In some embodiments, a formed ring is monocyclic. In some embodiments, a formed ring is saturated. In some embodiments, a formed ring is 3-membered. In some embodiments, a formed ring is 4-membered. In some embodiments, a formed ring is 5-membered. In some embodiments, a formed ring is 6-membered.

In some embodiments, X³ is a residue of an amino acid comprising a double bond, e.g., a terminal olefin, suitable for stapling. In some embodiments, X³ is a residue of an amino acid having the structure of A-II, A-III, etc. In some embodiments, X³ is a residue of RdN. In some embodiments, X³ is a residue of S8. In some embodiments, X³ is stapled. In some embodiments, X³ is stapled with X¹⁰.

In some embodiments, X³ is a residue of an amino acid having the structure of formula A-I, A-II, A-III, etc.

In some embodiments, X³ is a residue of an amino acid whose side chain is hydrophobic. In some embodiments, X³ is a residue of an amino acid whose side chain is an optionally substituted aliphatic group. In some embodiments, X³ is a residue of an amino acid whose side chain is optionally substituted C₁₋₁₀ alkyl. In some embodiments, X³ is a residue of an amino acid whose side chain is C₁₋₁₀ alkyl. In some embodiments, X³ is a residue of an amino acid whose side chain is C₁₋₁₀ alkyl optionally substituted with one or more non-polar and non-charged groups. In some embodiments, X³ is a residue of an amino acid whose side chain is C₁₋₁₀ alkyl optionally substituted with one or more substituents independently selected from halogen, —SR and —OR, where each R is independently C₁₋₄ alkyl. In some embodiments, X³ is a residue of an amino acid whose side chain is C₁₋₁₀ alkyl optionally substituted with one or more substituents independently selected from halogen, —SR and —OR, where each R is independently C₁₋₄ alkyl. In some embodiments, R is methyl. In some embodiments, X³ is a residue of Npg, Ala, Ile, Leu, Cha, Abu, hLeu, Val, F3CA, aIle, Nva, TOMe, S(Ome), nLeu, or HF2CA. In some embodiments, X³ is a residue of an amino acid whose side chain comprises an optionally substituted aromatic group. In some embodiments, X³ is a residue of an amino acid whose side chain comprises a hydrocarbon aromatic group. In some embodiments, X³ is a residue of NpG. Phe, 1NapA, or 2NapA.

In some embodiments, X³ is a residue of an amino acid whose side chain comprises a polar group, e.g., Gln, Hse, Ser, Asn, [AzAc]Lys, Thr, Asn, Ser, etc.

In some embodiments, X³ is a residue of Npg, Ala, Ile, Leu, Cha, Phe, Abu, hLeu, RdN, 1NapA, 2NapA, R8, Val, F3CA, [AzAc]Lys, Gln, aIle, Nva, TOMe, hSe, S(Ome), nLeu, Thr, Asn, Ser, or HF2CA.

In some embodiments, X³ is or comprises a residue of an amino acid or a moiety selected from Table A-I, Table A-II, Table A-III and Table A-IV.

In some embodiments, X³ is a residue of [lithocholate]-Lys, [lithocholate-PEG2]-Lys, [Me3AdamantC]-Lys, [Me3AdamantC-PEG2]-Lys, Npa, 1NapA, 2NapA, Abu, aIle, Ala, Asn, Asp, B5, Cha, F3CA, Gln, Glu, HF2CA, hLeu, hSe, Ile, iPrLys, Leu, Lys, nLeu, Npg, Nva, Phe, R8, RdN, S(Ome), Ser, TfeGA, Thr, TOMe, Trp, Tyr, or Val.

In some embodiments, X³ is a residue of [lithocholate]-Lys, [lithocholate-PEG2]-Lys, [Me3AdamantC]-Lys, [Me3AdamantC-PEG2]-Lys, or Npa.

In some embodiments, X³ is a residue of 1NapA, 2NapA, Abu, aIle, Ala, Asn, Asp, B5, Cha, F3CA, Gln, Glu, HF2CA, hLeu, hSe, Ile, iPrLys, Leu, Lys, nLeu, Npg, Nva, Phe, R8, RdN, S(Ome), Ser, TfeGA, Thr, TOMe, Trp, Tyr, or Val.

Various types of amino acid residues can be used for X⁴, e.g., a residue of an amino acid of formula A-I, A-II, A-III, A-IV, A-V, A-VI, etc. or a salt thereof in accordance with the present disclosure. In some embodiments, X⁴ is a residue of an amino acid of formula A-I, A-II, A-III, A-IV, A-V, A-VI, etc. or a salt thereof. In some embodiments, X⁴ is a residue of an amino acid of formula A-II or salt thereof. In some embodiments, X⁴ is a residue of an amino acid of formula A-III or salt thereof. In some embodiments, X⁴ is a residue of an amino acid of formula A-IV or salt thereof. In some embodiments, X⁴ is a residue of an amino acid of formula A-V or salt thereof. In some embodiments, X⁴ is a residue of an amino acid of formula A-VI or salt thereof. In some embodiments, X⁴ is —N(R^{a1})-L^{a1}-C(R^{a2})(R^{a3})-L^{a2}-C(O)—, wherein each variable is independently as described herein. In some embodiments, X⁴ is —N(R^{a1})—C(R^{a2})(R^{a3})—C(O)— wherein each variable is independently as described herein. In some embodiments, X⁴ is —N(R^{a1})—C(R^{a2})H—C(O)— wherein each variable is independently as described herein. In some embodiments, R^{a2} is -L^{a}-CH═CH₂, wherein L^{a} is as described herein. In some embodiments, R^{a3} is -L^{a}-CH═CH₂, wherein L^{a} is as described herein. In some embodiments, X⁴ is —N(R^{a1})-L^{a1}-C(-L^{a}-R^{SP1})(-L^{a}-R^{SP2})-L^{a2}-C(O)— wherein each variable is independently as described herein. In some embodiments, X⁴ is —N(R^{a1})—C(-L^{a}-R^{SP1}X-L^{a}-R^{SP2})—C(O)— wherein each variable is independently as described herein. In some embodiments, R^{a1} is —H. In some embodiments, R^{a3} is —H.

In some embodiments, each of R^{SP1} and R^{SP2} is or comprises independently optionally substituted —CH═CH₂. In some embodiments, each of R^{SP1} and R^{SP2} is independently —CH═CH₂. In some embodiments, each of -L^{a}-connected R^{SP1} or R^{SP2} is independent L as described herein. In some embodiments, L is an optionally substituted bivalent linear or branched C₁₋₁₀ hydrocarbon chain. In some embodiments, L is an optionally substituted bivalent linear C₁-10 hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)ₙ—, wherein n is 1-10. In some embodiments, L is —(CH₂)ₙ—, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(O)—, —N(R′)—, -Cy- or —O—. In some embodiments, L is an optionally substituted bivalent linear C₁₋₁₀ hydrocarbon chain wherein one or more methylene units of L are independently replaced with —C(O)—, —N(R′)—, -Cy- or —O—.

In some embodiments, X⁴ is residue of an amino acid suitable for stapling as described herein. In some embodiments, X⁴ is a residue of an amino acid which comprises two functional groups suitable for stapling. In some embodiments, X⁴ is a residue of an amino acid which comprises one and only one functional group suitable for stapling. In some embodiments, X⁴ is a residue of an amino acid which comprises two olefins, e.g., two terminal olefins. In some embodiments, X⁴ is a residue of an amino acid which comprises one and only one double bond for stapling, e.g., a terminal olefin. In some embodiments, X⁴ is a residue of an amino acid which has the structure of formula A-I, A-II, A-III, etc., wherein both R^{a2} and R^{a3} are independently -L^{a}-CH═CH₂, wherein each L^{a} is independently as described herein. In some embodiments, X⁴ is a residue of an amino acid which has the structure of formula A-I, A-II, A-III, etc., wherein only one of R^{a2} and R^{a3} is -L^{a}-CH═CH₂, wherein each L^{a} is independently as described herein. In some embodiments, each L^{a} is independently optionally substituted bivalent C₁₋₁₀ alkylene or heteroalkylene. In some embodiments, each L^{a} is independently optionally substituted —(CH₂)ₙ— wherein n is 1-10. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, X⁴ is B5. In some embodiments, X⁴ is R8, RdN, R5, RgN, ReN, R7, Az, R6, or R4.

In some embodiments, X⁴ is stapled. In some embodiments, X⁴ is connected to two residues independently through two staples (e.g., when X⁴ is B5). In some embodiments, X⁴ is staple with X¹, and X⁴ is stapled with X¹¹.

As described herein, various staples may be utilized for connecting stapled amino acid residues. In some embodiments, a staple is L^{s} as described herein. In some embodiments, each staple connected to X⁴ is independently L^{s} as described herein.

In some embodiments, L^{s} is -L^{s1}-L^{s2}-L^{s3}-, wherein each variable is independently as described herein. In some embodiments, one of L^{s1} and L^{s3} is L^{a} of one of two stapled amino acid residues, and the other is L^{a} of the other of two stapled amino acid residues. In some embodiments, L^{s3} is L^{a} of X⁴, e.g., when X⁴ is stapled with an amino acid residue to its N-terminus side (e.g., X¹). In some embodiments, L^{s1} is L^{a} of X⁴, e.g., when X⁴ is stapled with an amino acid residue to its C-terminus side (e.g., X¹). In some embodiments, L^{s1} is L^{a} of X¹, and L^{s3} is L^{a} of X⁴. In some embodiments, L^{s1} is L^{a} of X⁴, and L^{s3} is L^{a} of X¹. In some embodiments, two staples are bonded to X⁴, wherein a first staple staples X⁴ with an amino acid residue to the N-terminus side of X⁴ (an amino acid residue to a N-terminus side of a reference amino acid residue may be referred to as “N-direction amino acid residue” of the reference amino acid residue, e.g., X¹ is a N-direction amino acid residue of X⁴), wherein the first staple is L^{s}having the structure of -L^{s1}-L^{s2}-L^{s3}-, wherein L^{s1} is L^{a} of the N-direction amino acid residue, and L^{s3} is L^{a} of X⁴, and wherein a second staple staples X⁴ with an amino acid residue to the C-terminus side of X⁴ (an amino acid residue to a C-terminus side of a reference amino acid residue may be referred to as “C-direction amino acid residue” of the reference amino acid residue, e.g., X¹ is a C-direction amino acid residue of X⁴), wherein the second staple is L^{s} having the structure of -L^{s1}-L^{s2}-L^{s3}-, wherein L^{s3} is L^{a} of the C-direction amino acid residue, and L^{s1} is L^{a} of X⁴. Various embodiments of L^{a} are described herein and can be utilized for various amino acid residues including X⁴ and N-direction (e.g., X¹) and C-direction (e.g., X¹) amino acid residues. For example, in some embodiments, for X⁴ each L^{a} is —(CH₂)₃—.

As described herein, in some embodiments, L^{s2} is optionally substituted —CH═CH—. In some embodiments, L^{s2} is —CH═CH—. In some embodiments, L^{s2} is optionally substituted —CH₂—CH₂—. In some embodiments, L^{s2} is —CH₂—CH₂—.

In some embodiments, as described herein, each staple is independently bonded to two alpha carbon atoms of two stapled amino acid residues.

In some embodiments, X⁴ is stapled with two amino acid residues, e.g., X¹ and X¹¹. In some embodiments, X⁴ is stapled with only one residue, e.g., X¹¹ (e.g., when X⁴ is a residue of R5, R4, or R6). In some embodiments, X⁴ is —N(R^{a1})-L^{a1}-C(-L^{a}-CH═CH₂)(R^{a3})-L^{a2}-C(O)— wherein each variable is independently as described herein. In some embodiments, X⁴ is —N(R^{a1})—C(-L^{a}-CH═CH₂)(R^{a3})—C(O)— wherein each variable is independently as described herein. In some embodiments, X⁴ is a residue of R4. In some embodiments, X⁴ is a residue of R5. In some embodiments, X⁴ is a residue of R6.

In some embodiments, a staple is L^{s} as described herein. For example, in some embodiments, L^{s1} is L^{a} of a first amino acid residue of two stapled amino acid residues, e.g., X⁴, and L^{s3} is L^{a} of a second amino acid residue of two stapled amino acid residues, e.g., X¹¹, wherein a second amino acid residue (e.g., X¹) is a C-direction amino acid residue of a first amino acid residue (e.g., X⁴).

In some embodiments, X⁴ is stapled. In some embodiments, X⁴ is connected to two residues independently through two staples (e.g., when X⁴ is B5). In some embodiments, X⁴ is stapled with X¹ and X¹¹. In some embodiments, X⁴ is stapled with only one residue, e.g., X¹¹ (e.g., when X⁴ is R8, RdN, R5, RgN, ReN, R7, Az, R6, or R4).

In some embodiments, X⁴ is not stapled (e.g., when other residues are optionally stapled). In some embodiments, X⁴ is a residue of an amino acid whose side chain is hydrophobic, comprises an optionally substituted aromatic group, or comprises an acid group (e.g., —COOH, which as those skilled in the art appreciate may exist as a salt form at certain conditions, e.g., certain pH). In some embodiments, X⁴ is Ala. is X⁴ is Asp.

In some embodiments, X⁴ is selected from B5, R8, RdN, R5, Ala, RgN, ReN, R7, Az, Asp, R6, and R4.

In some embodiments, X⁴ is or comprises a residue of an amino acid or a moiety selected from Table A-I, Table A-II, Table A-III and Table A-IV.

In some embodiments, X⁴ is a residue of B3, B4, B6, Aib, Ala, Asp, Az, B5, Npg, R3, R4, R5, R6, R7, R8, RdN, ReN, RgN, S3, S4, S5, or S6.

In some embodiments, X⁴ is a residue of B3, B4, or B6.

In some embodiments, X⁴ is a residue of Aib, Ala, Asp, Az, B5, Npg, R3, R4, R5, R6, R7, R8, RdN, ReN, RgN, S3, S4, S5, or S6.

Various types of amino acid residues can be used for X⁵, e.g., a residue of an amino acid of formula A-I, A-II, A-III, A-IV, A-V, A-VI, etc. or a salt thereof in accordance with the present disclosure. In some embodiments, X⁵ is —N(R^{a1})-L^{a1}-C(R^{a2})(R^{a3})-L^{a2}-C(O)—, wherein each variable is independently as described herein. In some embodiments, X⁵ is —N(R^{a1})—C(R^{a2})(R^{a3})—C(O)—, wherein each variable is independently as described herein. In some embodiments, X⁵ is —N(R^{a1})—C(R^{a2})H—C(O)—, wherein each variable is independently as described herein. In some embodiments, R^{a1} is —H. In some embodiments, R^{a3} is —H.

In some embodiments, X⁵ is a residue of an amino acid of formula A-IV or a salt thereof. In some embodiments, X⁵ is a residue of an amino acid of formula PA, PA-a, PA-b, PA-c, or a salt thereof. In some embodiments, R^{PA} is —H and R^{PS} and R^{PC} are —OH. In some embodiments, X⁵ is —N(R^{a1})-L^{a1}-C(-L^{a}-COOH)(R^{a3})-L^{a2}-C(O)— wherein each variable is independently as described herein. In some embodiments, X⁵ is —N(R^{a1})—C(-L^{a}-COOH)(R^{a3})—C(O)— wherein each variable is independently as described herein. In some embodiments, L^{a} is L as described herein. For example, in some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)ₙ—, wherein n is 1-10. In some embodiments, L is —(CH₂)ₙ—, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is —CH(CH₃)—. In some embodiments, L is an optionally substituted bivalent linear or branched C₁₋₁₀ hydrocarbon chain wherein one or more methylene units of L are independently replaced with —C(O)—, —N(R′)—, -Cy- or —O—.

In some embodiments, X⁵ is a residue of amino acid that comprises an acidic or polar group. In some embodiments, X⁵ is a residue of amino acid whose side chain comprises an acidic group, e.g., a —COOH group or a salt form thereof (e.g., a compound of formula A-IV, etc.).

In some embodiments, X⁵ is a residue of Asp, Glu, RbGlu, SbGlu, NMeD, and isoDAsp. In some embodiments, X⁵ is a residue of Asp, Glu, RbGlu, SbGlu, and isoDAsp. In some embodiments, X⁵ is a residue of Asp. In some embodiments, X⁵ is a residue of Glu.

As appreciated by those skilled in the art, at physiological pH (about pH 7.4), an acidic group such as —COOH may exist, in some embodiments, predominantly, as its negatively charged form, e.g., —COO⁻.

In some embodiments, X⁵ is a residue of amino acid whose side chain comprises a polar group as described herein. In some embodiments, X⁵ is a residue of amino acid whose side chain comprises —OH. For example, in some embodiments, X⁵ is a residue of Hse. In some embodiments, X⁵ is a residue of Ser. In some embodiments, X⁵ is a residue of amino acid whose side chain comprises an amide group, e.g., —CONH₂. For example, in some embodiments, X⁵ is a residue of Asn. In some embodiments, X⁵ is a residue of Gln.

In some embodiments, X⁵ comprises a side chain which is hydrophobic, is aliphatic, is aromatic, etc.

In some embodiments, X⁵ is a residue of Asp, Hse, Asn, Glu, tetz, 3Thi, hPhe, 2pyrA, Ala, [MeSO2]Dap, [Tf]Dap, Ser, Gln, Leu, Dab, [MeSO2]Dab, nLeu, His, 3pyrA, 4pyrA, [NHiPr]AsnR, [NHEt]AsnR, [NHnPr]AsnR, [NHCyPr]AsnR, [NHCyBu]AsnR, [NHMe]AsnR, Phe, isoAsp, isoDAsp, RbGlu, and SbGlu. In some embodiments, X⁵ is selected from Asp, Asn, Gln, Glu, Hse, and Ser.

In some embodiments, X⁵ is or comprises a residue of an amino acid or a moiety selected from Table A-IV.

In some embodiments, X⁵ is a residue of [Et]AspE, [EtSSEt]AspE, [EtSSHex]AspE, [EtSSPh]AspE, [EtSSpy]AspE, [Me]AspE, AspSH, bMe2Asp, [MeSO2]Dab, [MeSO2]Dap, [NHCyBu]AsnR, [NHCyPr]AsnR, [NHEt]AsnR, [NHiPr]AsnR, [NHMe]AsnR, [NHnPr]AsnR, [Tf]Dap, 2pyrA, 3pyrA, 3Thi, 4pyrA, Ala, Arg, Asn, Asp, B5, BztA, Dab, Gln, Glu, His, hPhe, Hse, isoAsp, isoDAsp, Leu, nLeu, Npg, RbGlu, SbGlu, Ser, tetz, or Thr.

In some embodiments, X⁵ is a residue of [Et]AspE, [EtSSEt]AspE, [EtSSHex]AspE, [EtSSPh]AspE, [EtSSpy]AspE, [Me]AspE, AspSH, or bMe2Asp.

In some embodiments, X⁵ is a residue of [MeSO2]Dab, [MeSO2]Dap, [NHCyBu]AsnR, [NHCyPr]AsnR, [NHEt]AsnR, [NHiPr]AsnR, [NHMe]AsnR, [NHnPr]AsnR, [Tf]Dap, 2pyrA, 3pyrA, 3Thi, 4pyrA, Ala, Arg, Asn, Asp, B5, BztA, Dab, Gln, Glu, His, hPhe, Hse, isoAsp, isoDAsp, Leu, nLeu, Npg, RbGlu, SbGlu, Ser, tetz, or Thr.

Various types of amino acid residues can be used for X⁶, e.g., a residue of an amino acid of formula A-I, A-II, A-III, A-IV, A-V, A-VI, etc. or a salt thereof in accordance with the present disclosure. In some embodiments, X⁶ is a residue of an amino acid of formula A-I, A-II, A-III, A-IV, A-V, A-VI, etc. or a salt thereof. In some embodiments, X⁶ is —N(R^{a1})-L^{a1}-C(R^{a2})(R^{a3})-L^{a2}-C(O)—, wherein each variable is independently as described herein. In some embodiments, X⁶ is —N(R^{a1})—C(R^{a2})(R^{a3})—C(O)—, wherein each variable is independently as described herein. In some embodiments, X⁶ is —N(R^{a1})—C(R^{a2})H—C(O)—, wherein each variable is independently as described herein. In some embodiments, X⁶ is a residue of an amino acid of formula A-IV or a salt thereof. In some embodiments, X⁶ is a residue of an amino acid of formula PA, PA-a, PA-b, PA-c, or a salt thereof. In some embodiments, R^{PA} is —H and R^{PS} and R^{PC} are —OH. In some embodiments, R^{a1} is —H. In some embodiments, R^{a3} is —H.

In some embodiments, X⁶ is a residue of amino acid that comprises an acidic or polar group as described herein. In some embodiments, X⁶ is a residue of amino acid whose side chain comprises an acidic group, e.g., a —COOH group or a salt form thereof (e.g., a compound of formula A-IV, etc.) as described herein.

In some embodiments, X⁶ is a residue of an amino acid having the structure of formula A-IV or a salt thereof. In some embodiments, X⁶ is a residue of amino acid having the structure of formula PA, PA-a, PA-b, PA-c, etc. In some embodiments, R^{PA} is —H and R^{PS} and R^{PC} are —OH. In some embodiments, X⁶ is —N(R^{a1})-L^{a1}-C (-L^{a}-COOH)(R^{a3})-L^{a2}-C(O)—. In some embodiments, X⁶ is —NH-L^{a1}-C(-L^{a}-COOH)(R^{a3})-L^{a2}-C(O)—. In some embodiments, X⁶ is —NH—CH(-L^{a}-COOH)—C(O)—.

As described herein, L^{a} is L as described herein. In some embodiments, L is an optionally substituted bivalent linear or branched C₁₋₁₀ hydrocarbon chain wherein one or more methylene units of L are independently replaced with —C(O)—, —N(R′)—, -Cy- or —O—. In some embodiments, L is an optionally substituted bivalent linear C₁₋₁₀ hydrocarbon chain wherein one or more methylene units of L are independently replaced with —C(O)—, —N(R′)—, -Cy- or —O—. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)ₙ— wherein n is 1-10. In some embodiments, L is —(CH₂)ₙ—. In some embodiments, L is —CH₂—. In some embodiments, L is —(CH₂)₂—. In some embodiments, L is —(CH₂)₃—. In some embodiments, L is —(CH₂)₄—. In some embodiments, a methylene unit is replaced with -Cy-. In some embodiments, L is —CH₂-Cy-CH₂—. In some embodiments, L is —CH₂-Cy-. In some embodiments, L is —(CH₂)₄-Cy-CH₂—C(CH₃)₂—. In some embodiments, -Cy- is optionally substituted phenylene. In some embodiments, -Cy- is phenylene. In some embodiments, -Cy- is substituted phenylene. In some embodiments, -Cy- is mono-substituted phenylene. In some embodiments, a substituent is —F. In some embodiments, a substituent is optionally substituted C₁₋₆ alkyl. In some embodiments, a substituent is —CF₃. In some embodiments, a substituent is —OH. In some embodiments, phenylene is 1,2-phenylene. In some embodiments, phenylene is 1,3-phenylene. In some embodiments, phenylene is 1,4-phenylene. In some embodiments, a substituent is ortho to the carbon atom closed to —COOH. In some embodiments, it is meta. In some embodiments, it is para. In some embodiments, -Cy- is 1,3-phenylene (e.g., in 3COOHF). In some embodiments, -Cy- is an optionally substituted bivalent 5-10 membered heteroaryl group having 1-5 heteroatoms. In some embodiments, -Cy- is an optionally substituted bivalent 5-membered heteroaryl group having 1-4 heteroatoms. In some embodiments, -Cy- is an optionally substituted bivalent 6-membered heteroaryl group having 1-4 heteroatoms. In some embodiments, L is bonded to a backbone atom, e.g., an alpha carbon atom, at —CH₂—. In some embodiments, a methylene unit is replaced with —N(R′)— wherein R′ is as described herein. In some embodiments, L is —CH₂—N(R′)—CH₂— wherein R′ is as described herein. In some embodiments, R′ is R as described herein. In some embodiments, R is optionally substituted C₁₋₆ alkyl. In some embodiments, R is —CH₂CF₃.

In some embodiments, X⁶ is a residue of an amino acid of formula PA, PA-a, PA-b, PA-c, or a salt thereof, wherein R^{PA} is —H and R^{PS} and R^{PC} are —OH.

In some embodiments, X⁶ is a residue of TfeGA, 2COOHF, 3COOHF, Asp, Glu, RbGlu, SbGlu, NMeD, and isoDAsp. In some embodiments, X⁶ is a residue of TfeGA, 2COOHF, 3COOHF, Asp, Glu, RbGlu, SbGlu, and isoDAsp. In some embodiments, X⁶ is a residue of TfeGA. In some embodiments, X⁶ is a residue of 2COOHF. In some embodiments, X⁶ is a residue of 3COOHF. In some embodiments, X⁶ is a residue of Asp. In some embodiments, X⁶ is a residue of Glu. In some embodiments, X⁶ is a residue of EtGA. In some embodiments, X⁶ is a residue of 4COOHF. In some embodiments, X⁶ is a residue of Aad. In some embodiments, X⁶ is a residue of DGlu. In some embodiments, X⁶ is a residue of [iPr]GA. In some embodiments, X⁶ is a residue of [Pfbn]GA. In some embodiments, X⁶ is a residue of [Tfb]GA. In some embodiments, X⁶ is a residue of [Bn]GA. In some embodiments, X⁶ is a residue of lAcAw.

In some embodiments, X⁶ is a residue of amino acid whose side chain comprises a polar group as described herein. In some embodiments, X⁶ is a residue of amino acid whose side chain comprises —OH. For example, in some embodiments, X⁶ is a residue of Hse. In some embodiments, X⁶ is a residue of Ser. In some embodiments, X⁶ is a residue of Thr. In some embodiments, X⁶ is a residue of amino acid whose side chain comprises an amide group, e.g., —CONH₂. For example, in some embodiments, X⁶ is a residue of Asn. In some embodiments, X⁶ is a residue of Gln. In some embodiments, X⁶ is a residue of Cit.

In some embodiments, X⁶ is a hydrophobic amino acid residue as described herein. In some embodiments, X⁶ comprises a side chain which is hydrophobic, is aliphatic, comprises an optionally substituted aromatic group, comprises a basic group, etc.

As those skilled in the art reading the present disclosure will readily appreciate, amino acid residues of certain properties, structures, etc. described for one position may also be utilized at other positions where amino acid residues of the same properties, structures, etc. can be utilized. For example, when hydrophobic amino acid residues can be utilized at both positions X³ and X⁶, hydrophobic amino acid residues described for X³ can be utilized for X⁶ and vice versa. Similarly, when acidic amino acid residues can be utilized at positions X², X⁵ and X⁶, acidic amino acid residues described for one of them may be utilized at the other two positions as well.

In some embodiments, X⁶ comprises a side chain comprising an optionally substituted aromatic group as described herein.

In some embodiments, X⁶ is or comprises a residue of an amino acid or a moiety selected from Table A-IV.

In some embodiments, X⁶ is a residue of Asp, Glu, TfeGA, Thr, EtGA, Asn, 3COOHF, HIs, Gln, 2NapA, 4COOHF, nLeu, Leu, Cit, Aad, Cha, hLeu, hPhe, Ala, 3PyrA, Bip, Tyr, aMeDF, Phe, 1NapA, DaMeL, 3F3MeF, 4F3MeF, tetz, Arg, 2COOHF, DGlu, BztA, Trp, 6F1NapA, 3FF, 4FF, 34FF, 2PyrA, 4PyrA, hTyr, Qui, DipA, 4AmPhe, 2Thi, 1meH, [iPr]GA, [Pfbn]GA, [Tfb]GA, [Bn]GA, Lys, [Tfp]Dap, 1AcAW, Ser, Val, and [MeSO2]Dap.

In some embodiments, X⁶ is a residue of [2COOH4NH2Ph]Dap, [2COOH4NO2Ph]Cys, [2COOH4NO2Ph]Dap, [2COOHPh]TriAzDab, [2COOHPh]TriAzDap, [2Nic]Dap, [3COOH4NO2Ph]Dap, [4AcMePip]GA, [4AcMePip]GAbu, [4CF3PhAc]GA, [4CF3PhAc]GAbu, [Ac]GA, [Ac]GAbu, [Bn]GAbu, [CCpCO2H]TriAzDap, [CF3CO]GA, [CH₂CChCO2H]TriAzDap, [CH₂CCpCO2H]TriAzDap, [CH₂CH₂CO2H]TriAzDab, [CH₂CH₂CO2H]TriAzDap, [CH₂CMe2CO2H]TriAzDab, [CH₂CMe2CO2H]TriAzDap, [CH₂CO2H]GAbu, [CH₂CO2H]TriAzDab, [CH₂CO2H]TriAzDap, [CMe2CO2H]TriAzDab, [CMe2CO2H]TriAzDap, [Et]AspE, [Et]GA, [Et]GAbu, [Et]GluE, [EtSSEt]AspE, [EtSSEt]GluE, [EtSSHex]AspE, [EtSSPh]AspE, [EtSSPh]GluE, [EtSSpy]GluE, [Me]AspE, [Me]GA, [Me]GluE, [MeMorphBz]GA, [MeMorphBz]GAbu, [MePipAc]GA, [MePipAc]GAbu, [MorphAc]GA, [MorphAc]GAbu, [MorphEt]GAbu, [NdiMeButC]GA, [NdiMeButC]GAbu, [Pfb]GA, [Pfbn]GAbu, [PfBz]GA, [PfBz]GAbu, [PfPhAc]GA, [PfPhAc]GAbu, [Pic]GA, [Pic]GAbu, [sBu]GA, [Tfb]GAbu, [Tfp]GA, [Tfp]GAbu, 3TzF, 4F3COOHF, 4TzF, 5F3Me3COOHF, 5iPr3COOHF, AspSH, GAbu, GluSH, R2COOPipA, R3COOPipA, S2COOPipA, S3COOPipA, [Bn]GA, [iPr]GA, [Me2NPr]GA, [Me2NPr]GAbu, [MeSO2]Dap, [Pfbn]GA, [Tfb]GA, [Tfp]Dap, 1AcAW, 1meH, 1NapA, 2COOHF, 2NapA, 2PyrA, 2Thi, 34FF, 3cbmf, 3COOHF, 3F3MeF, 3FF, 3PyrA, 3thi, 4AmPhe, 4COOHF, 4F3MeF, 4FF, 4PyrA, 6F1NapA, Aad, Ala, aMeDF, Arg, Asn, Asp, B5, Bip, BztA, Cha, Cit, DaMeL, DGlu, DipA, EtGA, GA, Gln, Glu, His, hLeu, hPhe, Hse, hTyr, Leu, Lys, nLeu, Npg, Pff, Phe, Qui, Ser, tetz, TfeGA, Thr, Trp, Tyr, or Val.

In some embodiments, X⁶ is a residue of [2COOH4NH2Ph]Dap, [2COOH4NO2Ph]Cys, [2COOH4NO2Ph]Dap, [2COOHPh]TriAzDab, [2COOHPh]TriAzDap, [2Nic]Dap, [3COOH4NO2Ph]Dap, [4AcMePip]GA, [4AcMePip]GAbu, [4CF3PhAc]GA, [4CF3PhAc]GAbu, [Ac]GA, [Ac]GAbu, [Bn]GAbu, [CCpCO2H]TriAzDap, [CF3CO]GA, [CH₂CChCO2H]TriAzDap, [CH₂CCpCO2H]TriAzDap, [CH₂CH₂CO2H]TriAzDab, [CH₂CH₂CO2H]TriAzDap, [CH₂CMe2CO2H]TriAzDab, [CH₂CMe2CO2H]TriAzDap, [CH₂CO2H]GAbu, [CH₂CO2H]TriAzDab, [CH₂CO2H]TriAzDap, [CMe2CO2H]TriAzDab, [CMe2CO2H]TriAzDap, [Et]AspE, [Et]GA, [Et]GAbu, [Et]GluE, [EtSSEt]AspE, [EtSSEt]GluE, [EtSSHex]AspE, [EtSSPh]AspE, [EtSSPh]GluE, [EtSSpy]GluE, [Me]AspE, [Me]GA, [Me]GluE, [MeMorphBz]GA, [MeMorphBz]GAbu, [MePipAc]GA, [MePipAc]GAbu, [MorphAc]GA, [MorphAc]GAbu, [MorphEt]GAbu, [NdiMeButC]GA, [NdiMeButC]GAbu, [Pfb]GA, [Pfbn]GAbu, [PfBz]GA, [PfBz]GAbu, [PfPhAc]GA, [PfPhAc]GAbu, [Pic]GA, [Pic]GAbu, [sBu]GA, [Tfb]GAbu, [Tfp]GA, [Tfp]GAbu, 3TzF, 4F3COOHF, 4TzF, 5F3Me3COOHF, 5iPr3COOHF, AspSH, GAbu, GluSH, R2COOPipA, R3COOPipA, S2COOPipA, or S3COOPipA.

In some embodiments, X⁶ is a residue of [Bn]GA, [iPr]GA, [Me2NPr]GA, [Me2NPr]GAbu, [MeSO2]Dap, [Pfbn]GA, [Tfb]GA, [Tfp]Dap, 1AcAW, 1meH, 1NapA, 2COOHF, 2NapA, 2PyrA, 2Thi, 34FF, 3cbmf, 3COOHF, 3F3MeF, 3FF, 3PyrA, 3thi, 4AmPhe, 4COOHF, 4F3MeF, 4FF, 4PyrA, 6F1NapA, Aad, Ala, aMeDF, Arg, Asn, Asp, B5, Bip, BztA, Cha, Cit, DaMeL, DGlu, DipA, EtGA, GA, Gln, Glu, His, hLeu, hPhe, Hse, hTyr, Leu, Lys, nLeu, Npg, Pff, Phe, Qui, Ser, tetz, TfeGA, Thr, Trp, Tyr, or Val.

Various types of amino acid residues can be used for X⁷, e.g., a residue of an amino acid of formula A-I, A-II, A-III, A-IV, A-V, A-VI, etc. or a salt thereof in accordance with the present disclosure. In some embodiments, X⁷ is —N(R^{a1})-L^{a1}-C(R^{a2})(R^{a3})-L^{a2}-C(O)—, wherein each variable is independently as described herein. In some embodiments, X⁷ is —N(R^{a1})—C(R^{a2})(R^{a3})—C(O)—, wherein each variable is independently as described herein. In some embodiments, X⁷ is —N(R^{a1})—C(R^{a2})H—C(O)—, wherein each variable is independently as described herein. In some embodiments, R^{a1} is —H. In some embodiments, R^{a3} is —H.

In some embodiments, R^{a2} is R, wherein R is C₁₋₁₀ aliphatic. In some embodiments, R^{a3} is R, wherein R is C₁₋₁₀ aliphatic. In some embodiments, each of R^{a2} and R^{a3} is independently R as described herein. In some embodiments, R^{a2} and R^{a3} are the same. In some embodiments, R is C₁₋₁₀ alkyl. In some embodiments, R is methyl.

In some embodiments, X⁷ is a residue of an amino acid whose side chain is hydrophobic. In some embodiments, X⁷ is a hydrophobic amino acid residue described herein, e.g., those described for X³. In some embodiments, X⁷ is a residue of an amino acid whose side chain is optionally substituted C₁₋₁₀ alkyl. In some embodiments, X⁷ is a residue of an amino acid whose side chain is C₁₋₁₀ alkyl. In some embodiments, X⁷ is a residue of an amino acid whose side chain is C₁₋₁₀ alkyl optionally substituted with one or more non-polar and non-charged groups. In some embodiments, X⁷ comprises a side chain comprising a cycloaliphatic group (e.g., a 3-, 4-, 5-, or 6-membered cycloalkyl group).

Various types of amino acid residues can be utilized for X⁷. In some embodiments, X⁷ comprises a polar side chain. In some embodiments, X⁷ comprises a non-polar side chain. In some embodiments, X⁷ comprises a hydrophobic side chain. In some embodiments, X⁷ comprises an aliphatic side chain. In some embodiments, X⁷ comprises an alkyl side chain. In some embodiments, X⁷ comprises a side chain comprising an optionally substituted aromatic group. In some embodiments, X⁷ comprises a side chain comprising an acidic group, e.g., —COOH. In some embodiments, X⁷ comprises a side chain comprising a basic group, e.g., —N(R)₂. In some embodiments, X⁷ comprises a detectable moiety such as a fluorescent moiety.

In some embodiments, X⁷ is a residue of amino acid whose side chain comprises a polar group. Various polar amino acid residues described herein may be utilized for X⁷. In some embodiments, X⁷ is a residue of amino acid whose side chain comprises —OH. For example, in some embodiments, X⁷ is a residue of Ser. In some embodiments, X⁷ is a residue of amino acid whose side chain comprises a basic group. In some embodiments, X⁷ is a residue of amino acid whose side chain comprises an amino group, e.g., Lys. In some embodiments, X⁷ comprises a side chain comprising an optionally substituted aromatic group, e.g., Phe.

In some embodiments, X⁷ is selected from Ala, Leu, iPrLys, Phe, Ser, Aib, Gln, nLeu, Trp, Ile, and Lys, and a substituted or labeled lysine. In some embodiments, X⁷ is selected from Ala, Leu, iPrLys, [AzAc]Lys, Phe, Ser, [FAM6Ppg][p1 TB]Lys, Aib, Gln, nLeu, Trp, [FAM6Ppg][1TriAc]Lys, Ile, and Lys. In some embodiments, a lysine is labeled with a detectable moiety (either directly or indirectly detectable). In some embodiments, X⁷ is selected from Ala, Leu, iPrLys, Phe, Ser, Aib, Gln, nLeu, Trp, Ile, and Lys. In some embodiments, X⁷ is Ala.

In some embodiments, X⁷ is or comprises a residue of an amino acid or a moiety of Table A-IV.

In some embodiments, X⁷ is a residue of [20xoPpz]GlnR, [3Py]4SF, [4Pippip]GlnR, [Ac]Lys, [AcPpz]GlnR, [bismethoxyethylamine]GlnR, [CF3CO]Lys, [CH₂NMe2]4SEF, [EtSO2Ppz]GlnR, [isoindoline]GlnR, [Me2diaminobutane]GlnR, [Me2Npr]Lys, [Me2NPrPip]GlnR, [MeSO2]Lys, [Morph]GlnR, [NHEt]GlnR, [NMe2]GlnR, [Phc]Lys, [TfePpz]GlnR, Cpg, CyLeu, F2PipNva, hhLeu, Me2Asn, Me2Gln, MeGln, MePpzAsn, Met2O, MorphAsn, MorphNva, [MorphAc]Lys, [mPEG4]Lys, 3COOHF, Aib, Ala, Asn, Asp, Gln, Gly, His, Hse, Ile, iPrLys, Leu, Lys, nLeu, Phe, R5, Ser, Thr, or Trp.

In some embodiments, X⁷ is a residue of [20xoPpz]GlnR, [3Py]4SF, [4Pippip]GlnR, [Ac]Lys, [AcPpz]GlnR, [bismethoxyethylamine]GlnR, [CF3CO]Lys, [CH₂NMe2]4SEF, [EtSO2Ppz]GlnR, [isoindoline]GlnR, [Me2diaminobutane]GlnR, [Me2Npr]Lys, [Me2NPrPip]GlnR, [MeSO2]Lys, [Morph]GlnR, [NHEt]GlnR, [NMe2]GlnR, [Phc]Lys, [TfePpz]GlnR, Cpg, CyLeu, F2PipNva, hhLeu, Me2Asn, Me2Gln, MeGln, MePpzAsn, Met2O, MorphAsn, or MorphNva.

In some embodiments, X⁷ is a residue of [MorphAc]Lys, [mPEG4]Lys, 3COOHF, Aib, Ala, Asn, Asp, Gln, Gly, His, Hse, Ile, iPrLys, Leu, Lys, nLeu, Phe, R5, Ser, Thr, or Trp.

Various types of amino acid residues can be used for X⁸, e.g., a residue of an amino acid of formula A-I, A-II, A-III, A-IV, A-V, A-VI, etc. or a salt thereof in accordance with the present disclosure. In some embodiments, X⁸ is —N(R^{a1})-L^{a1}-C(R^{a2})(R^{a3})-L^{a2}-C(O)—, wherein each variable is independently as described herein. In some embodiments, X⁸ is —N(R^{a1})—C(R^{a2})(R^{a3})—C(O)—, wherein each variable is independently as described herein. In some embodiments, X⁸ is —N(R^{a1})—C(R^{a2})H—C(O)—, wherein each variable is independently as described herein. In some embodiments, R^{a1} is —H. In some embodiments, R^{a3} is —H.

In some embodiments, X⁸ comprises a polar side chain as described herein. In some embodiments, X⁸ comprises a non-polar side chain. In some embodiments, X⁸ comprises a hydrophobic side chain. In some embodiments, X⁸ is a hydrophobic amino acid residue as described herein, e.g., those described for X³. In some embodiments, X⁸ comprises an aliphatic side chain. In some embodiments, X⁸ comprises an alkyl side chain. In some embodiments, X⁸ comprises a side chain comprising an optionally substituted aromatic group. In some embodiments, X⁸ comprises a side chain comprising an acidic group, e.g., —COOH, as described herein. In some embodiments, X⁸ comprises a side chain comprising a basic group, e.g., —N(R)₂ as described herein. In some embodiments, X⁸ comprises a detectable moiety such as a fluorescent moiety.

In some embodiments, Xx is selected from Ala, Leu, Phe, Ser, Aib, Asp, Glu, Aad, Trp, nLeu, Gln, Ile, Lys, iPrLys, and a substituted or labeled lysine. In some embodiments, a lysine is labeled with a detectable moiety (either directly or indirectly detectable). In some embodiments, X⁸ is selected from Ala, Leu, Phe, Ser, Aib, Asp, Glu, Aad, Trp, nLeu, [mPEG2]Lys, [AzAc]Lys, Gln, [FAM6Ppg][1TriAc]Lys, [35CF3PhPr]Lys, [1NapPr]Lys, [22PhPr]Lys, [MorphAc]Lys, [MePipAc]Lys, [MeBipipAc]Lys, [4MePipBz]Lys, [MeMorphBz]Lys, [Me2NCBz]Lys, [mPEG4]Lys, [mPEG6]Lys, [mPEG8]Lys, [Bua]Lys, [Oct]Lys, [AdamantC]Lys, [Me3AdamantC]Lys, [AdamantPro]Lys, Ile, Lys, and iPrLys. In some embodiments, XX⁸ is Ala.

In some embodiments, XX⁸ is or comprises a residue of an amino acid or a moiety selected from Table A-IV.

In some embodiments, XX⁸ is a residue of [lithocholate]-Lys, [lithocholate-PEG2]-Lys, [Me2NCBz]Lys, [Me3AdamantC-PEG2]-Lys, F2PipAbu, F2PipNva, MePpzAbu, MePpzAsn, MePpzNva, MorphAbu, MorphAsn, MorphNva, dAla, [1NapPr]Lys, [22PhPr]Lys, [35CF3PhPr]Lys, [4MePipBz]Lys, [AdamantC]Lys, [AdamantPro]Lys, [Bua]Lys, [Me3AdamantC]Lys, [Me3AdamantC]-Lys, [MeBipipAc]Lys, [MeMorphBz]Lys, [MePipAc]Lys, [MorphAc]Lys, [mPEG2]Lys, [mPEG4]Lys, [mPEG6]Lys, [mPEG8]Lys, [Oct]Lys, 3COOHF, Aad, Aib, Ala, Asp, Gln, Glu, Gly, Ile, iPrLys, Leu, Lys, nLeu, Phe, Ser, or Trp.

In some embodiments, XX⁸ is a residue of [lithocholate]-Lys, [lithocholate-PEG2]-Lys, [Me2NCBz]Lys, [Me3AdamantC-PEG2]-Lys, F2PipAbu, F2PipNva, MePpzAbu, MePpzAsn, MePpzNva, MorphAbu, MorphAsn, MorphNva, or dAla.

In some embodiments, XX⁸ is a residue of [1NapPr]Lys, [22PhPr]Lys, [35CF3PhPr]Lys, [4MePipBz]Lys, [AdamantC]Lys, [AdamantPro]Lys, [Bua]Lys, [Me3AdamantC]Lys, [Me3AdamantC]-Lys, [MeBipipAc]Lys, [MeMorphBz]Lys, [MePipAc]Lys, [MorphAc]Lys, [mPEG2]Lys, [mPEG4]Lys, [mPEG6]Lys, [mPEG8]Lys, [Oct]Lys, 3COOHF, Aad, Aib, Ala, Asp, Gln, Glu, Gly, Ile, iPrLys, Leu, Lys, nLeu, Phe, Ser, or Trp.

Various types of amino acid residues can be used for X⁹, e.g., a residue of an amino acid of formula A-I, A-II, A-III, A-IV, A-V, A-VI, etc. or a salt thereof in accordance with the present disclosure. In some embodiments, X⁹ is —N(R^{a1})-L^{a1}-C(R^{a2})(R^{a3})-L^{a2}-C(O)—, wherein each variable is independently as described herein. In some embodiments, X⁹ is —N(R^{a1})—C(R^{a2})(R^{a3})—C(O)—, wherein each variable is independently as described herein. In some embodiments, X⁹ is —N(R^{a1})—C(R^{a2})H—C(O)—, wherein each variable is independently as described herein. In some embodiments, R^{a1} is —H. In some embodiments, R^{a3} is —H.

In some embodiments, X⁹ comprises a side chain which is or comprises an optionally substituted aromatic group. In some embodiments, X⁹ is an aromatic amino acid residue as described herein. In some embodiments, an aromatic group is optionally substituted 5-membered heteroaryl having 1-3 heteroatoms. In some embodiments, an aromatic group is optionally substituted 5-membered heteroaryl having 1-3 nitrogen atoms. In some embodiments, an aromatic group is optionally substituted 5-membered heteroaryl having one sulfur atom. In some embodiments, an aromatic group is optionally substituted phenyl. In some embodiments, X⁹ comprises a side chain which is or comprises an optionally substituted aromatic group, wherein each substituent of the aromatic group is independently selected from halogen, —OR, —R, —C(O)OH, or —CN, wherein each R is independently hydrogen or C₁₋₄ alkyl or haloalkyl. In some embodiments, an aromatic group is phenyl. In some embodiments, an aromatic group is optionally substituted 8-10 membered bicyclic aryl or heteroaryl having 1-5 heteroatoms. In some embodiments, X⁹ comprises a side chain which is or comprises an optionally substituted aromatic group, wherein each substituent of the aromatic group is independently halogen. In some embodiments, X⁹ comprises a side chain which is or comprises two optionally substituted aromatic groups. In some embodiments, X⁹ comprises a side chain which is or comprises an optionally substituted aromatic group, wherein each substituent of the aromatic group is independently selected from halogen or —OH. In some embodiments, an aromatic group is phenyl. In some embodiments, an aromatic group is optionally substituted 8-10 membered bicyclic aryl or heteroaryl having 0-5 heteroatoms. In some embodiments, an aromatic group is optionally substituted 9-10 membered bicyclic aryl or heteroaryl having one heteroatom. In some embodiments, X⁹ is a residue of an amino acid of formula A-I or a salt thereof. In some embodiments, an amino acid residue has the structure of —NH—C(R^{a2})(R^{a3})—C(O)— or a salt thereof. In some embodiments, an amino acid residue has the structure of —NH—CH(R^{a3})—C)O)— or a salt thereof. As described herein, R^{a3} is -L^{a}-R′ wherein each variable is independently as described herein. In some embodiments, R′ is R as described herein. In some embodiments, R is an optionally substituted group selected from phenyl, 10-membered bicyclic aryl, 5−6 membered heteroaryl having 1-4 heteroatoms, and 9-10 membered bicyclic heteroaryl having 1-5 heteroatoms. In some embodiments, each substituent is independently halogen or —OH or C₁₋₆ haloaliphatic. In some embodiments, each substituent is independently halogen or —OH. In some embodiments, R is optionally substituted phenyl. In some embodiments, R is phenyl. In some embodiments, R is optionally substituted aryl. In some embodiments, R is aryl. In some embodiments, R is optionally substituted 5-membered heteroaryl having 1-4 heteroatoms. In some embodiments, R is optionally substituted 5-membered heteroaryl having 1 heteroatom. In some embodiments, optionally substituted R is 6-membered heteroaryl having 1-4 heteroatoms. In some embodiments, optionally substituted R is 6-membered heteroaryl having 1 heteroatom. In some embodiments, R is optionally substituted 9-membered heteroaryl having 1-5 heteroatoms. In some embodiments, R is optionally substituted 9-membered heteroaryl having 1 heteroatom. In some embodiments, R is optionally substituted 10-membered heteroaryl having 1-5 heteroatoms. In some embodiments, R is optionally substituted 10-membered heteroaryl having 1 heteroatom. In some embodiments, a heteroatom is nitrogen. In some embodiments, a heteroatom is oxygen. In some embodiments, a heteroatom is sulfur. As described herein, L^{a} is L. In some embodiments, L is a covalent bond. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)ₙ—, wherein n is 1-10. In some embodiments, L is —(CH₂)ₙ—, wherein n is 1-10. In some embodiments, L is —CH₂—. In some embodiments, L is —(CH₂)₂—. In some embodiments, Lis —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—.

In some embodiments, X⁹ is a residue of an amino acid having the structure of formula A-I, wherein R^{a2} is -L^{a}-R′, and R′ is an optionally substituted aromatic group. In some embodiments, L^{a} is optionally substituted CH₂. In some embodiments, L^{a} is —CH₂—. In some embodiments, X⁹ comprises a side chain which is or comprises an optionally substituted aromatic group, wherein each substituent of the aromatic group is independently selected from halogen, —OR, —R, —N(R)₂, —C(O)N(R)₂, or —CN, wherein each R is independently —H, C₁₋₄ alkyl or haloalkyl, or -Ph. In some embodiments, X⁹ comprises a side chain which is or comprises an optionally substituted aromatic group, wherein each substituent of the aromatic group is independently selected from halogen, —OR, —R, —NH₂, —C(O)NH₂, -Ph, or —CN, wherein each R is independently C₁₋₄ alkyl or haloalkyl. In some embodiments, X⁹ comprises a side chain which is or comprises an optionally substituted aromatic group, wherein each substituent of the aromatic group is independently selected from halogen, —OR, —R, —NH₂, —C(O)NH₂, -Ph, or —CN, wherein each R is independently C₁₋₂ alkyl or haloalkyl. In some embodiments, X⁹ comprises a side chain which is or comprises an optionally substituted aromatic group, wherein each substituent of the aromatic group is independently selected from halogen, —OR, —R, NH₂, —C(O)NH₂, -Ph, or —CN, wherein each R is independently methyl optionally substituted with one or more halogen. In some embodiments, X⁹ comprises a side chain which is or comprises an optionally substituted aromatic group, wherein each substituent of the aromatic group is independently selected from —F, —OR, —CH₃, —NH₂, —C(O)NH₂, -Ph, or —CN, wherein each R is independently methyl optionally substituted with one or more —F. In some embodiments, X⁹ comprises a side chain which is or comprises an optionally substituted aromatic group, wherein each substituent of the aromatic group is independently selected from —F, —OR, —CH₃, —CF₃, —NH₂, —C(O)NH₂, -Ph, or —CN. In some embodiments, X⁹ comprises a side chain which is or comprises an optionally substituted aromatic group, wherein each substituent of the aromatic group is independently selected from halogen, —CH₃, —CF₃, or —CN. In some embodiments, X⁹ comprises a side chain which is or comprises an aromatic group optionally substituted at 2′-position. In some embodiments, X⁹ comprises a side chain which is or comprises an unsubstituted aromatic group. In some embodiments, an aromatic group is optionally substituted 5-membered heteroaryl having 1-3 heteroatoms. In some embodiments, an aromatic group is optionally substituted 5-membered heteroaryl having 1-3 nitrogen atoms. In some embodiments, an aromatic group is optionally substituted 5-membered heteroaryl having one oxygen atom. In some embodiments, an aromatic group is optionally substituted 5-membered heteroaryl having one sulfur atom. In some embodiments, an aromatic group is optionally substituted phenyl. In some embodiments, an aromatic group is optionally substituted 8-10 membered bicyclic aryl or heteroaryl having 1-5 heteroatoms. In some embodiments, X⁹ is selected from Phe, 3F3MeF, 2Thi, 3Thi, 4F3MeF, 30MeF, 3MeF, 2MeF, 2NapA, 345FF, 34FF, 3FF, His, 2FurA, 2PyrA, 4AmPhe, 4FF, 1meH, 23FF, 2FF, 35FF, 3CBMF, 3ClF, 3meH, 3PyrA, 4CBMF, 4ClF, 4Thz, BztA, hPhe, hTyr, MeTyr, 1NapA, 2CNF, 3CNF, 4CNF, 4MeF, Bip, DipA, and Phg. In some embodiments, X⁹ is Phe.

In some embodiments, X⁹ comprises a polar side chain. In some embodiments, X⁹ is a polar amino acid residue as described herein. In some embodiments, X⁹ comprises a non-polar side chain. In some embodiments, X⁹ comprises a hydrophobic side chain. In some embodiments, X⁹ is a hydrophobic amino acid residue as described herein. In some embodiments, X⁹ comprises an aliphatic side chain. In some embodiments, X⁹ comprises an alkyl side chain. In some embodiments, X⁹ comprises a side chain comprising a cycloaliphatic group (e.g., a 5- or 6-membered cycloalkyl group). In some embodiments, X⁹ comprises a side chain comprising an optionally substituted aromatic group. In some embodiments, X⁹ comprises a side chain comprising an acidic group, e.g., —COOH. In some embodiments, X⁹ is an acidic amino acid residue as described herein. In some embodiments, X⁹ comprises a side chain comprising a basic group, e.g., —N(R)₂. In some embodiments, X⁹ is a basic amino acid residue as described herein. In some embodiments, X⁹ is Gln. In some embodiments, X⁹ is Asp. In some embodiments, X⁹ is Cha. In some embodiments, X⁹ is CypA. In some embodiments, X⁹ is Ala. In some embodiments, X⁹ is nLeu. In some embodiments, X⁹ is Npg.

In some embodiments, X⁹ is or comprises a residue of an amino acid or a moiety selected from Table A-IV.

In some embodiments, X⁹ is a residue of [3Py]4SF, [CH2NMe2]4SEF, [CH2NMe2]TriAzDap, [CH2Ppz]TriAzDap, [MorphCH2]TriAzDap, [SO2MorphCH2]TriAzDap, 2NH2F, 3CH2NMe2F, 3CO2PhF, 3SO2F, 4BrF, Cba, 1meH, 1NapA, 23FF, 2cbmf, 2ClF, 2CNF, 2FF, 2FurA, 2MeF, 2NapA, 2PyrA, 2Thi, 345FF, 34FF, 35FF, 3CBMF, 3ClF, 3CNF, 3COOHF, 3F3MeF, 3FF, 3MeF, 3meH, 30MeF, 3PyrA, 3Thi, 4AmPhe, 4CBMF, 4ClF, 4CNF, 4F3MeF, 4FF, 4MeF, 4Thz, Ala, Asp, Bip, BztA, Cha, CypA, DipA, Gln, His, hPhe, hTyr, MeTyr, nLeu, Npg, Phe, Phg, Ser, or Tyr.

In some embodiments, X⁹ is a residue of [3Py]4SF, [CH2NMe2]4SEF, [CH2NMe2]TriAzDap, [CH2Ppz]TriAzDap, [MorphCH2]TriAzDap, [SO2MorphCH2]TriAzDap, 2NH2F, 3CH2NMe2F, 3CO2PhF, 3SO2F, 4BrF, or Cba.

In some embodiments, X⁹ is a residue of 1meH, 1NapA, 23FF, 2cbmf, 2ClF, 2CNF, 2FF, 2FurA, 2MeF, 2NapA, 2PyrA, 2Thi, 345FF, 34FF, 35FF, 3CBMF, 3ClF, 3CNF, 3COOHF, 3F3MeF, 3FF, 3MeF, 3meH, 30MeF, 3PyrA, 3Thi, 4AmPhe, 4CBMF, 4ClF, 4CNF, 4F3MeF, 4FF, 4MeF, 4Thz, Ala, Asp, Bip, BztA, Cha, CypA, DipA, Gln, His, hPhe, hTyr, MeTyr, nLeu, Npg, Phe, Phg, Ser, or Tyr.

Various types of amino acid residues can be used for X¹⁰, e.g., a residue of an amino acid of formula A-I, A-II, A-III, A-IV, A-V, A-VI, etc. or a salt thereof in accordance with the present disclosure. In some embodiments, X¹⁰ is —N(R^{a1})-L^{a1}-C(R^{a2})(R^{a3})-L^{a2}-C(O)—, wherein each variable is independently as described herein. In some embodiments, X¹⁰ is —N(R^{a1})—C(R^{a2})(R^{a3})—C(O)—, wherein each variable is independently as described herein. In some embodiments, X¹⁰ is —N(R^{a1})—C(R^{a2})H—C(O)—, wherein each variable is independently as described herein. In some embodiments, R^{a1} is —H. In some embodiments, R^{a3} is —H.

In some embodiments, X¹⁰ is a residue of an amino acid suitable for stapling as described herein. In some embodiments, X¹⁰ is a residue of an amino acid comprising a double bond, e.g., a terminal olefin, suitable for stapling. In some embodiments, X¹⁰ is a residue of an amino acid having the structure of A-II, A-III, etc. In some embodiments, X¹⁰ is a residue of RdN. In some embodiments, X¹⁰ is a residue of S8. In some embodiments, X¹⁰ is stapled. In some embodiments, X¹⁰ is stapled with X³.

In some embodiments, X¹⁰ is a residue of an amino acid having the structure of formula A-I, A-II, A-III, etc.

In some embodiments, X¹⁰ is a residue of an amino acid whose side chain is hydrophobic. In some embodiments, X¹⁰ is a residue of an amino acid whose side chain is an optionally substituted aliphatic group. In some embodiments, X¹⁰ is a residue of an amino acid whose side chain is optionally substituted C₁₋₁₀ alkyl. In some embodiments, X¹⁰ is a residue of an amino acid whose side chain is C₁₋₁₀ alkyl. In some embodiments, X¹⁰ is a residue of an amino acid whose side chain is C₁₋₁₀ alkyl optionally substituted with one or more non-polar and non-charged groups. In some embodiments, X¹⁰ is a residue of an amino acid whose side chain is C₁₋₁₀ alkyl optionally substituted with one or more substituents independently selected from halogen, —SR and —OR, where each R is independently C₁₋₄ alkyl. In some embodiments, X¹⁰ is a residue of an amino acid whose side chain is C₁₋₁₀ alkyl optionally substituted with one or more substituents independently selected from halogen, —SR and —OR, where each R is independently C₁₋₄ alkyl. In some embodiments, R is methyl. In some embodiments, X¹⁰ is a residue of Npg, Ala, Ile, Leu, Cha, Abu, hLeu, Val, F3CA, aIle, Nva, TOMe, S(Ome), nLeu, or HF2CA. In some embodiments, X¹⁰ is a residue of an amino acid whose side chain comprises an optionally substituted aromatic group. In some embodiments, X¹⁰ is a residue of an amino acid whose side chain comprises a hydrocarbon aromatic group. In some embodiments, X¹⁰ is a residue of NpG. Phe, 1NapA, or 2NapA. In some embodiments, X¹⁰ is a residue of Leu.

In some embodiments, X¹⁰ is a residue of amino acid whose side chain comprises a polar group as described herein. In some embodiments, X¹⁰ is a residue of amino acid whose side chain comprises —OH. For example, in some embodiments, X¹⁰ is a residue of Hse. In some embodiments, X¹⁰ is a residue of Ser. In some embodiments, X¹⁰ is a residue of Thr. In some embodiments, X¹⁰ is a residue of amino acid whose side chain comprises an amide group, e.g., —CONH₂. For example, in some embodiments, X¹⁰ is a residue of Asn. In some embodiments, X¹⁰ is a residue of Gln. In some embodiments, X¹⁰ is a residue of Cit.

In some embodiments, X¹⁰ is a residue of amino acid whose side chain comprises an optionally substituted aromatic group. In some embodiments, X¹⁰ is an aromatic amino acid residue as described herein. In some embodiments, an aromatic group is optionally substituted phenyl. In some embodiments, X¹⁰ is Phe.

In some embodiments, X¹⁰ is selected from Asn, Val, Gln, Leu, Thr, Ser, Phe, Ala, Hse, Cit, iPrLys, S7, S5, Cha, PyrS, S(Ome), [AzAc]Lys, nLeu, 2F3MeF, 3F3MeF, and 4F3MeF. In some embodiments, X¹⁰ is a residue of Asn, Val, Gln, Leu, Thr, Ser, Phe, Ala, Hse, Cit, iPrLys, S7, S5, Cha, PyrS, S(Ome), or [AzAc]Lys. In some embodiments, X¹⁰ is Leu, Thr or Hse. In some embodiments, X¹⁰ is Leu. In some embodiments, X¹⁰ is Thr. In some embodiments, X¹⁰ is Hse.

In some embodiments, X¹⁰ is or comprises a residue of an amino acid or a moiety selected from Table A-I, Table A-II, Table A-III and Table A-IV.

In some embodiments, X¹⁰ is a residue of [4MePpzPip]GlnR, [4Pippip]GlnR, [4PyPip]GlnR, [CH₂NMe2]TriAzDap, [CH₂Ppz]TriAzDap, [H4IAP]GlnR, [Me2NPrPip]GlnR, [Morph]GlnR, [MorphCH2]TriAzDap, [NHBn]GlnR, [Ppz]GlnR, [RDMAPyr]GlnR, [SO2MorphCH2]TriAzDap, [TfePpz]GlnR, 4BrF, AcLys, F2PipNva, Me2Asn, Me2Gln, MePpzAsn, MorphAsn, MorphGln, MorphNva, MeAsn, 2F3MeF, 3F3MeF, 4F3MeF, Abu, Ala, Arg, Asn, Cha, Cit, dSer, Gln, His, hLeu, Hse, iPrLys, Leu, Lys, nLeu, Npg, Phe, PyrS, PyrS2, R5, S(Ome), S5, S7, Ser, Thr, Trp, or Val.

In some embodiments, X¹⁰ is a residue of [4MePpzPip]GlnR, [4Pippip]GlnR, [4PyPip]GlnR, [CH₂NMe2]TriAzDap, [CH₂Ppz]TriAzDap, [H4IAP]GlnR, [Me2NPrPip]GlnR, [Morph]GlnR, [MorphCH2]TriAzDap, [NHBn]GlnR, [Ppz]GlnR, [RDMAPyr]GlnR, [SO2MorphCH2]TriAzDap, [TfePpz]GlnR, 4BrF, AcLys, F2PipNva, Me2Asn, Me2Gln, MePpzAsn, MorphAsn, MorphGln, MorphNva, or MeAsn.

In some embodiments, X¹⁰ is a residue of 2F3MeF, 3F3MeF, 4F3MeF, Abu, Ala, Arg, Asn, Cha, Cit, dSer, Gln, His, hLeu, Hse, iPrLys, Leu, Lys, nLeu, Npg, Phe, PyrS, PyrS2, R5, S(Ome), S5, S7, Ser, Thr, Trp, or Val.

Various types of amino acid residues can be used for X¹¹, e.g., a residue of an amino acid of formula A-I, A-II, A-III, A-IV, A-V, A-VI, etc. or a salt thereof in accordance with the present disclosure. In some embodiments, X¹¹ is —N(R^{a1})-L^{a1}-C(R^{a2})(R^{a3})-L^{a2}-C(O)—, wherein each variable is independently as described herein. In some embodiments, X¹¹ is —N(R^{a1})—C(R^{a2})(R^{a3})—C(O)—, wherein each variable is independently as described herein. In some embodiments, X¹¹ is —N(R^{a1})—C(R^{a2})H—C(O)—, wherein each variable is independently as described herein. In some embodiments, R^{a1} is —H. In some embodiments, R^{a3} is —H.

In some embodiments, X¹¹ is a residue of an amino acid suitable for stapling as described herein. In some embodiments, an amino acid residue suitable for stapling is —N(R^{a1})-L^{a1}-C(-L^{a}-R^{SP1})(R^{a3})-L^{a2}-C(O)— wherein each variable is independently as described herein. In some embodiments, it is —N(R^{a1})—C(-L^{a}-R^{SP1})(R^{a3})—C(O)— wherein each variable is independently as described herein. In some embodiments, in a pair of amino acid residues suitable for stapling, each amino acid residue is independently —N(R^{a1})-L^{a1}-C(-L^{a}-R^{SP1})(R^{a3})-L^{a2}-C(O)— or —N(R^{a1})—C(-L^{a}-R^{SP1})(R^{a3})—C(O)—, wherein each variable is independently as described herein. In some embodiments, R^{a1} is —H. In some embodiments, R^{a3} is —H. In some embodiments, both R^{a1} and R^{a3} are —H. In some embodiments, R^{SP1} comprises optionally substituted —CH═CH—. In some embodiments, R^{SP1} is or comprises optionally substituted —CH═CH₂. In some embodiments, R^{SP1} is —CH═CH₂.

In some embodiments, X¹¹ is a residue of an amino acid suitable for stapling. In some embodiments, X¹¹ is a residue of an amino acid, e.g., having the structure of formula A-I, A-II, A-III, A-IV, A-V, A-VI, etc., whose side chain comprise a functional group suitable for stapling, e.g., a double bond. In some embodiments, X¹¹ is a residue of an amino acid that comprises one and no more than one functional groups for stapling. In some embodiments, X¹¹ is a residue of an amino acid that comprises one and no more than one double bond for stapling. As in certain embodiments of X¹¹, in some embodiments, X¹¹ comprises a ring structure, and its amino group is part of a ring. In some embodiments, X¹¹ is an amino acid as described herein (e.g., of formula A-I, A-II, A-III, etc.), wherein R^{a1} and R^{a3} are taken together to form an optionally substituted ring, e.g., an optionally substituted 3-10 membered ring. In some embodiments, R^{a1} and R^{a3} are taken together with their intervening atoms to form an optionally substituted 3-10 membered saturated or partially saturated ring having, in addition to the intervening atoms, 0-5 heteroatoms.

In some embodiments, R^{a2} and R^{a3} are taken together to form an optionally substituted ring, e.g., an optionally substituted 3-10 membered ring. In some embodiments, R^{a2} and R^{a3} are taken together with their intervening atoms to form an optionally substituted 3-10 membered saturated or partially saturated ring having, in addition to the intervening atoms, 0-5 heteroatoms.

As described herein, in some embodiments, a formed ring, e.g., by R^{a1} and R^{a3} taken together with their intervening atoms, by R^{a2} and R^{a3} taken together with their intervening atoms, or by any other two suitable R taken together with their intervening atoms, either in X¹¹ or another moiety, is saturated. In some embodiments, a formed ring is monocyclic. In some embodiments, a formed ring has no heteroatoms in addition to the intervening atoms. In some embodiments, a formed ring has at least one heteroatom in addition to the intervening atoms. In some embodiments, a formed ring has at least one nitrogen in addition to the intervening atoms. In some embodiments, L^{a1} and L^{a2} are covalent bond. In some embodiments, a formed ring is unsubstituted. In some embodiments, a formed ring is substituted. In some embodiments, a substitute comprises a double bond which is suitable for metathesis with another double bond to form a staple. In some embodiments, a substituent has the structure of —C(O)—O—(CH₂)ₙ—Ch=CH₂, wherein n is 1, 2, 3, 4, 5, 6, 7, 8, 9, or 10. In some embodiments, a substituent bonds to a nitrogen ring atom (e.g., see PyrS, PyrsS1, PyrS2, PyrS3, etc.).

In some embodiments, L^{a} is —(CH₂)ₙ₁—N(R′)—C(O)—(CH₂)ₙ₂—, wherein each variable is independently as described herein, and each —CH₂— is optionally substituted. In some embodiments, L^{a} is —(CH₂)ₙ₁—N(R′)—C(O)—(CH₂)ₙ₂—, wherein each variable is independently as described herein. In some embodiments, —(CH₂)ₙ₁— is bonded to X¹¹. In some embodiments, n1 is 1, 2, 3, 4, 5, 6, 7, 8, 9, or 10. In some embodiments, n1 is 1. In some embodiments, n1 is 2. In some embodiments, n1 is 3. In some embodiments, n2 is 1, 2, 3, 4, 5, 6, 7, 8, 9, or 10. In some embodiments, n2 is 1. In some embodiments, n2 is 2. In some embodiments, n2 is 3. In some embodiments, n2 is 4. In some embodiments, n2 is 5. In some embodiments, R′ of —N(R′)— of L^{a} and R^{a3} are taken together with their intervening atoms to form an optionally substituted ring. In some embodiments, a formed ring is optionally substituted 3-10 membered monocyclic, saturated or partially unsaturated ring having, in addition to the nitrogen atom to which R′ is attached, 0-3 heteroatoms. In some embodiments, a formed ring is saturated. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring has no ring heteroatoms other than the nitrogen atom to which R′ is attached. In some embodiments, X¹¹ is a residue of PyrS2.

In some embodiments, X¹¹ is stapled. In some embodiments, X¹¹ is stapled with X⁴. In some embodiments, X¹¹ is PyrS2 and stapled.

In some embodiments, a staple, e.g., L^{s}, has the structure of -L^{s1}-L^{s2}-L^{s3}, wherein each variable is independently as described herein. In some embodiments, L^{s1} or L^{s3} is L^{a} of X¹¹ as described herein. In some embodiments, L^{s3} is L^{a} of X¹¹ as described herein. In some embodiments, L^{s1} is L^{a} of another amino acid residue, e.g., X⁴. In some embodiments, L^{s1} is L as described herein. In some embodiments, L is an optionally substituted bivalent linear or branched C₁₋₁₀ hydrocarbon chain. In some embodiments, L is an optionally substituted bivalent linear C1-10 hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)ₙ—, wherein n is 1-10. In some embodiments, L is —(CH₂)ₙ—, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—. In some embodiments, L^{s3} is L as described herein. In some embodiments, L^{s3} is —(CH₂)ₙ₁— N(R′)—C(O)—(CH₂)ₙ₂—, wherein each variable is independently as described herein, and each —CH₂— is optionally substituted. In some embodiments, L^{s3} is —(CH₂)ₙ₁— N(R′)—C(O)—(CH₂)ₙ₂—, wherein each variable is independently as described herein. In some embodiments, —(CH₂)ₙ₁— is bonded to X¹¹. In some embodiments, n1 is 1, 2, 3, 4, 5, 6, 7, 8, 9, or 10. In some embodiments, n1 is 1. In some embodiments, n1 is 2. In some embodiments, n1 is 3. In some embodiments, n2 is 1, 2, 3, 4, 5, 6, 7, 8, 9, or 10. In some embodiments, n2 is 1. In some embodiments, n2 is 2. In some embodiments, n2 is 3. In some embodiments, n2 is 4. In some embodiments, n2 is 5. In some embodiments, R′ of —N(R′)— of L^{a} and R^{a1} are taken together with their intervening atoms to form an optionally substituted ring. In some embodiments, a formed ring is optionally substituted 3-10 membered monocyclic, saturated or partially unsaturated ring having, in addition to the nitrogen atom to which R′ is attached, 0-3 heteroatoms. In some embodiments, a formed ring is saturated. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring has no ring heteroatoms other than the nitrogen atom to which R′ is attached.

In some embodiments, L^{s2} is optionally substituted —CH═CH—. In some embodiments, L^{s2} is —CH═CH—. In some embodiments, L^{s2} is optionally substituted —CH₂—CH₂—. In some embodiments, L^{s2} is —CH₂—CH₂—.

In some embodiments, X¹¹ is or comprises a residue of an amino acid or a moiety selected from Table A-I, Table A-II, Table A-III and Table A-IV.

In some embodiments, X¹¹ is a residue of an amino acid selected from PyrS2, S8, PyrS, S7, PyrS3, SeN, Az, S4, S6, SdN, S10, S5, SgN or PyrS1. In some embodiments, X¹¹ is a residue of PyrS2. In some embodiments, X¹¹ is a residue of S8. In some embodiments, X¹¹ is a residue of PyrS. In some embodiments, X¹¹ is a residue of S7. In some embodiments, X¹¹ is a residue of PyrS3. In some embodiments, X¹¹ is a residue of SeN. In some embodiments, X¹¹ is a residue of Az. In some embodiments, X¹¹ is a residue of S4. In some embodiments, X¹¹ is a residue of S6. In some embodiments, X¹¹ is a residue of SdN. In some embodiments, X¹¹ is a residue of S10. In some embodiments, X¹¹ is a residue of S5. In some embodiments, X¹¹ is a residue of SgN. In some embodiments, X¹ is a residue of PyrS1.

In some embodiments, X¹¹ is stapled. In some embodiments, X¹¹ is stapled with X⁴.

In some embodiments, X¹¹ is amino acid residue not suitable for stapling, e.g., via olefin metathesis. In some embodiments, X¹¹ comprises a polar side chain. In some embodiments, X¹¹ comprises anon-polar side chain. In some embodiments, X¹¹ comprises a hydrophobic side chain. In some embodiments, X¹¹ comprises an aliphatic side chain. In some embodiments, X¹¹ comprises an alkyl side chain. In some embodiments, X¹¹ comprises a side chain comprising an optionally substituted aromatic group. In some embodiments, X¹¹ comprises a side chain comprising an acidic group, e.g., —COOH. In some embodiments, X¹¹ comprises a side chain comprising a basic group, e.g., —N(R)₂. In some embodiments, X¹¹ comprises a detectable moiety such as a fluorescent moiety. In some embodiments, X¹¹ is Ala. In some embodiments, X¹¹ is Phe.

In some embodiments, X¹¹ is selected from S8, PyrS2, PyrS, S7, PyrS3, SeN, Ala, Az, Phe, S4, S6, SdN, S10, S5, SgN, and PyrS1.

In some embodiments, X¹ is a residue of Az2, Az3, PyrR2, PyrS4, SeNc5, SPip1, SPip2, SPip3, Aib, Ala, Az, Leu, Phe, PyrS, PyrS1, PyrS2, PyrS3, S10, S4, S5, S6, S7, S8, SdN, SeN, or SgN.

In some embodiments, X¹ is a residue of Az2, Az3, PyrR2, PyrS4, SeNc5, SPip1, SPip2, or SPip3.

In some embodiments, X¹ is a residue of Aib, Ala, Az, Leu, Phe, PyrS, PyrS1, PyrS2, PyrS3, S10, S4, S5, S6, S7, S8, SdN, SeN, or SgN.

Various types of amino acid residues can be used for X², e.g., a residue of an amino acid of formula A-I, A-II, A-III, A-IV, A-V, A-VI, etc. or a salt thereof in accordance with the present disclosure. In some embodiments, X¹² is —N(R^{a1})-L^{a1}-C(R^{a2})(R^{a3})-L^{a2}-C(O)—, wherein each variable is independently as described herein. In some embodiments, X² is —N(R^{a1})—C(R^{a2})(R^{a3})—C(O)—, wherein each variable is independently as described herein. In some embodiments, X¹² is —N(R^{a1})—C(R^{a2})H—C(O)—, wherein each variable is independently as described herein. In some embodiments, R^{a1} is —H. In some embodiments, R^{a3} is —H.

In some embodiments, X¹² comprises a side chain which is or comprises an optionally substituted aromatic group. In some embodiments, X¹² is an aromatic amino acid residue as described herein. In some embodiments, an aromatic group is optionally substituted 5-membered heteroaryl having 1-3 heteroatoms. In some embodiments, an aromatic group is optionally substituted 5-membered heteroaryl having 1-3 nitrogen atoms. In some embodiments, an aromatic group is optionally substituted 5-membered heteroaryl having one oxygen atom. In some embodiments, an aromatic group is optionally substituted 5-membered heteroaryl having one sulfur atom. In some embodiments, an aromatic group is optionally substituted 6-membered heteroaryl having 1-3 heteroatoms. In some embodiments, an aromatic group is optionally substituted 6-membered heteroaryl having 1 nitrogen atom. In some embodiments, an aromatic group is optionally substituted phenyl. In some embodiments, X¹² comprises a side chain which is or comprises an optionally substituted aromatic group, wherein each substituent of the aromatic group is independently selected from halogen, —OR, —R, —C(O)OH, —C(O)NH₂, —CN, or —NO₂, wherein each R is independently C₁₋₄ alkyl or haloalkyl. In some embodiments, an aromatic group is phenyl. In some embodiments, an aromatic group is optionally substituted 8-10 membered bicyclic aryl or heteroaryl having 1-5 heteroatoms. In some embodiments, X¹² comprises a side chain which is or comprises an optionally substituted aromatic group, wherein each substituent of the aromatic group is independently halogen. In some embodiments, X¹² comprises a side chain which is or comprises two optionally substituted aromatic groups. In some embodiments, X¹² comprises a side chain which is or comprises an optionally substituted aromatic group, wherein each substituent of the aromatic group is independently selected from halogen or —OH. In some embodiments, an aromatic group is phenyl. In some embodiments, an aromatic group is optionally substituted 8-10 membered bicyclic aryl or heteroaryl having 0-5 heteroatoms. In some embodiments, an aromatic group is optionally substituted 9-10 membered bicyclic aryl or heteroaryl having one heteroatom. In some embodiments, X¹² is a residue of an amino acid of formula A-I or a salt thereof. In some embodiments, an amino acid residue has the structure of —NH—C(R^{a2})(R^{a3})—C(O)— or a salt thereof. In some embodiments, an amino acid residue has the structure of —NH—CH(R^{a3})—C)O)— or a salt thereof. As described herein, R^{a3} is -L^{a}-R′ wherein each variable is independently as described herein. In some embodiments, R′ is R as described herein. In some embodiments, R is an optionally substituted group selected from phenyl, 10-membered bicyclic aryl, 5−6 membered heteroaryl having 1-4 heteroatoms, and 9-10 membered bicyclic heteroaryl having 1-5 heteroatoms. In some embodiments, each substituent is independently halogen or —OH or C₁₋₆ haloaliphatic. In some embodiments, each substituent is independently halogen or —OH. In some embodiments, R is optionally substituted phenyl. In some embodiments, R is phenyl. In some embodiments, R is optionally substituted aryl. In some embodiments, R is aryl. In some embodiments, R is optionally substituted 5-membered heteroaryl having 1-4 heteroatoms. In some embodiments, R is optionally substituted 5-membered heteroaryl having 1 heteroatom. In some embodiments, optionally substituted R is 6-membered heteroaryl having 1-4 heteroatoms. In some embodiments, optionally substituted R is 6-membered heteroaryl having 1 heteroatom. In some embodiments, R is optionally substituted 9-membered heteroaryl having 1-5 heteroatoms. In some embodiments, R is optionally substituted 9-membered heteroaryl having 1 heteroatom. In some embodiments, R is optionally substituted 10-membered heteroaryl having 1-5 heteroatoms. In some embodiments, R is optionally substituted 10-membered heteroaryl having 1 heteroatom. In some embodiments, a heteroatom is nitrogen. In some embodiments, a heteroatom is oxygen. In some embodiments, a heteroatom is sulfur. As described herein, L^{a} is L. In some embodiments, L is a covalent bond. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)ₙ—, wherein n is 1-10. In some embodiments, L is —(CH₂)ₙ—, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—.

In some embodiments, X¹² is a residue of an amino acid having the structure of formula A-I, wherein R^{a2} is -L^{a}-R′, and R′ is an optionally substituted aromatic group. In some embodiments, L^{a} is optionally substituted CH₂. In some embodiments, L^{a} is —CH₂—. In some embodiments, X¹² comprises a side chain which is or comprises an optionally substituted aromatic group, wherein each substituent of the aromatic group is independently selected from halogen, —OR, —R, —N(R)₂, —C(O)N(R)₂, or —CN, wherein each R is independently —H, C₁₋₄ alkyl or haloalkyl, or -Ph. In some embodiments, X¹² comprises a side chain which is or comprises an optionally substituted aromatic group, wherein each substituent of the aromatic group is independently selected from halogen, —OR, —R, —NH₂, —C(O)NH₂, -Ph, or —CN, wherein each R is independently C₁₋₄ alkyl or haloalkyl. In some embodiments, X¹² comprises a side chain which is or comprises an optionally substituted aromatic group, wherein each substituent of the aromatic group is independently selected from halogen, —OR, —R, —NH₂, —C(O)NH₂, -Ph, or —CN, wherein each R is independently C₁₋₂ alkyl or haloalkyl. In some embodiments, X¹² comprises a side chain which is or comprises an optionally substituted aromatic group, wherein each substituent of the aromatic group is independently selected from halogen, —OR, —R, NH₂, —C(O)NH₂, -Ph, or —CN, wherein each R is independently methyl optionally substituted with one or more halogen. In some embodiments, X¹² comprises a side chain which is or comprises an optionally substituted aromatic group, wherein each substituent of the aromatic group is independently selected from —F, —OR, —CH₃, —NH₂, —C(O)NH₂, -Ph, or —CN, wherein each R is independently methyl optionally substituted with one or more —F. In some embodiments, X¹² comprises a side chain which is or comprises an optionally substituted aromatic group, wherein each substituent of the aromatic group is independently selected from —F, —OR, —CH₃, —CF₃, —NH₂, —C(O)NH₂, -Ph, or —CN. In some embodiments, X¹² comprises a side chain which is or comprises an optionally substituted aromatic group, wherein each substituent of the aromatic group is independently selected from halogen, —CH₃, —CF₃, or —CN. In some embodiments, X¹² comprises a side chain which is or comprises an aromatic group optionally substituted at 2′-position. In some embodiments, X¹² comprises a side chain which is or comprises an unsubstituted aromatic group. In some embodiments, an aromatic group is optionally substituted 5-membered heteroaryl having 1-3 heteroatoms. In some embodiments, an aromatic group is optionally substituted 5-membered heteroaryl having 1-3 nitrogen atoms. In some embodiments, an aromatic group is optionally substituted 5-membered heteroaryl having one oxygen atom. In some embodiments, an aromatic group is optionally substituted 5-membered heteroaryl having one sulfur atom. In some embodiments, an aromatic group is optionally substituted phenyl. In some embodiments, an aromatic group is optionally substituted 8-10 membered bicyclic aryl or heteroaryl having 1-5 heteroatoms. In some embodiments, X¹² is selected from Phe, 3Thi, 2ClF, 3FF, 20MeF, 2FF, Pff, 2CBMF, 3ClF, 3F3MeF, 1NapA, 2NapA, 2PyrA, 4CBMF, 4COOHF, 4F3MeF, Tyr, 2BrF, 2F3MeF, 2Thi, 4PyrA, hPhe, Trp, 1meH, 23FF, 2MeF, 34FF, 30MeF, 3PyrA, 4ClF, 4CNF, His, 2CNF, 2NO2F, 35FF, 3CBMF, 3CNF, 3MeF, 3meH, 4FF, 4MeF, 4Thz, BztA, dPhe, and hTyr. In some embodiments, X² is 3Thi. In some embodiments, X¹² is Phe. In some embodiments, X¹² is Phe, wherein the phenyl group is substituted. In some embodiments, X¹² is Phe, wherein the phenyl group is 2′-substituted. In some embodiments, X¹² is 1FF. In some embodiments, X¹² is 2ClF. In some embodiments, X¹² is 2BrF. In some embodiments, X¹² is 2F3MeF. In some embodiments, X¹² is 2MeF. In some embodiments, X¹² is 2CNF.

In some embodiments, X¹² comprises a polar side chain. In some embodiments, X¹² comprises a non-polar side chain. In some embodiments, X¹² comprises a hydrophobic side chain. In some embodiments, X¹² comprises an aliphatic side chain. In some embodiments, X¹² comprises an alkyl side chain. In some embodiments, X¹² comprises a side chain comprising a cycloaliphatic group (e.g., a 5- or 6-membered cycloalkyl group). In some embodiments, X¹² comprises a side chain comprising an optionally substituted aromatic group. In some embodiments, X¹² comprises a side chain comprising an acidic group, e.g., —COOH. In some embodiments, X¹² comprises a side chain comprising a basic group, e.g., —N(R)₂. In some embodiments, X¹² is Gln. In some embodiments, X¹² is Asn. In some embodiments, X¹² is Asp. In some embodiments, X¹² is Glu. In some embodiments, X¹² is Cha. In some embodiments, X¹² is CypA. In some embodiments, X¹² is Ala. In some embodiments, X¹² is nLeu. In some embodiments, X¹² is Npg. In some embodiments, X¹² is [Acryl]Dap.

In some embodiments, X¹² is a polar amino acid residue as described herein. In some embodiments, X¹² is hydrophobic amino acid residue as described herein. In some embodiments, X¹² is a hydrophobic amino acid residue as described herein.

In some embodiments, X¹² is a residue of amino acid that comprises an acidic or polar group. In some embodiments, X¹² is a residue of amino acid whose side chain comprises an acidic group, e.g., a —COOH group or a salt form thereof (e.g., a compound of formula A-IV, etc.). Various acidic amino acid residues described herein may be utilized for X¹², e.g., those described for X², X⁵, X⁶, etc. In some embodiments, X¹² is 2COOHF. In some embodiments, X¹² is a residue of amino acid whose side chain comprises a polar group. In some embodiments, X² is a residue of amino acid whose side chain comprises an amide group, e.g., —C(O)N(R′)₂ such as —CONH₂. For example, in some embodiments, X¹² is a residue of 2cbmF. Various other polar amino acid residues described herein may also be utilized for X¹².

In some embodiments, X¹² is selected from Phe, 3Thi, 2ClF, 3FF, 20MeF, 2FF, Pff, Asp, 2CBMF, 3ClF, 3F3MeF, 1NapA, 2NapA, 2PyrA, 4CBMF, 4COOHF, 4F3MeF, Tyr, 2BrF, 2F3MeF, 2Thi, 4PyrA, Cha, CypA, hPhe, Trp, dPhe, [Acryl]Dap, 1meH, 23FF, 2MeF, 34FF, 30MeF, 3PyrA, 4ClF, 4CNF, Ala, Glu, His, 2CNF, 2NO2F, 35FF, 3CBMF, 3CNF, 3MeF, 3meH, 4FF, 4MeF, 4Thz, Asn, BztA, dPhe and hTyr.

In some embodiments, X¹² is or comprises a residue of an amino acid or a moiety selected from Table A-IV.

In some embodiments, X¹² is a residue of [CyPr]-3SF, [Ph]3SF, [Ph]-3SF, 3BrF, 3CBMF, Cba, [Acryl]Dap, 1meH, 1NapA, 23FF, 2BrF, 2CBMF, 2ClF, 2CNF, 2F3MeF, 2FF, 2MeF, 2NapA, 2N02F, 20MeF, 2pyrA, 2Thi, 34FF, 35FF, 3ClF, 3CNF, 3F3MeF, 3FF, 3MeF, 3meH, 30MeF, 3PyrA, 3thi, 4CBMF, 4ClF, 4CNF, 4COOHF, 4F3MeF, 4FF, 4MeF, 4PyrA, 4Thz, Ala, Asn, Asp, BztA, Cha, CypA, dPhe, Gln, Glu, His, hPhe, hTyr, Leu, Npg, Pff, Phe, PyrS2, Trp, or Tyr.

In some embodiments, X¹² is a residue of [CyPr]-3SF, [Ph]3SF, [Ph]-3SF, 3BrF, 3CBMF, or Cba.

In some embodiments, X¹² is a residue of [Acryl]Dap, 1meH, 1NapA, 23FF, 2BrF, 2CBMF, 2ClF, 2CNF, 2F3MeF, 2FF, 2MeF, 2NapA, 2N02F, 20MeF, 2pyrA, 2Thi, 34FF, 35FF, 3ClF, 3CNF, 3F3MeF, 3FF, 3MeF, 3meH, 30MeF, 3PyrA, 3thi, 4CBMF, 4ClF, 4CNF, 4COOHF, 4F3MeF, 4FF, 4MeF, 4PyrA, 4Thz, Ala, Asn, Asp, BztA, Cha, CypA, dPhe, Gln, Glu, His, hPhe, hTyr, Leu, Npg, Pff, Phe, PyrS2, Trp, or Tyr.

Various types of amino acid residues can be used for X¹³, e.g., a residue of an amino acid of formula A-I, A-II, A-III, A-IV, A-V, A-VI, etc. or a salt thereof in accordance with the present disclosure. In some embodiments, X¹³ is —N(R^{a1})-L^{a1}-C(R^{a2})(R^{a3})-L^{a2}-C(O)—, wherein each variable is independently as described herein. In some embodiments, X¹³ is —N(R^{a1})—C(R^{a2})(R^{a3})—C(O)—, wherein each variable is independently as described herein. In some embodiments, X¹³ is —N(R^{a1})—C(R^{a2})H—C(O)—, wherein each variable is independently as described herein. In some embodiments, R^{a1} is —H. In some embodiments, R^{a3} is —H.

Various types of amino acid residues can be used for X¹³, e.g., a residue of an amino acid of formula A-I, A-II, A-III, A-IV, A-V, A-VI, etc. or a salt thereof in accordance with the present disclosure. In some embodiments, X¹³ is —N(R^{a1})-L^{a1}-C(R^{a2})(R^{a3})-L^{a2}-C(O)—, wherein each variable is independently as described herein. In some embodiments, X¹³ is —N(R^{a1})—C(R^{a2})(R^{a3})—C(O)—, wherein each variable is independently as described herein. In some embodiments, X¹³ is —N(R^{a1})—C(R^{a2})H—C(O)—, wherein each variable is independently as described herein. In some embodiments, R^{a1} is —H. In some embodiments, R^{a3} is —H.

In some embodiments, X¹³ comprises a side chain which is or comprises an optionally substituted aromatic group. In some embodiments, X¹³ is an aromatic amino acid residue as described herein.

In some embodiments, X¹³ is a residue of an amino acid having the structure of formula A-I, wherein R^{a2} is -L^{a}-R′, and R′ is an optionally substituted aromatic group. In some embodiments, an aromatic group is optionally substituted 5-membered heteroaryl having 1-3 heteroatoms. In some embodiments, an aromatic group is optionally substituted 5-membered heteroaryl having 1-3 nitrogen atoms. In some embodiments, an aromatic group is optionally substituted 5-membered heteroaryl having one sulfur atom. In some embodiments, an aromatic group is optionally substituted phenyl. In some embodiments, X¹³ comprises a side chain which is or comprises an optionally substituted aromatic group, wherein each substituent of the aromatic group is independently selected from halogen, —OR, —R, —C(O)OH, or —CN, wherein each R is independently hydrogen or C₁₋₄ alkyl or haloalkyl. In some embodiments, an aromatic group is phenyl. In some embodiments, an aromatic group is optionally substituted 8-10 membered bicyclic aryl or heteroaryl having 1-5 heteroatoms. In some embodiments, X¹³ comprises a side chain which is or comprises an optionally substituted aromatic group, wherein each substituent of the aromatic group is independently halogen. In some embodiments, X¹³ comprises a side chain which is or comprises two optionally substituted aromatic groups. In some embodiments, X¹³ comprises a side chain which is or comprises an optionally substituted aromatic group, wherein each substituent of the aromatic group is independently selected from halogen or —OH. In some embodiments, an aromatic group is phenyl. In some embodiments, an aromatic group is optionally substituted 8-10 membered bicyclic aryl or heteroaryl having 0-5 heteroatoms. In some embodiments, an aromatic group is optionally substituted 9-10 membered bicyclic aryl or heteroaryl having one heteroatom. In some embodiments, X¹³ is a residue of an amino acid of formula A-I or a salt thereof. In some embodiments, an amino acid residue has the structure of —NH—C(R^{a2})(R^{a3})—C(O)— or a salt thereof. In some embodiments, an amino acid residue has the structure of —NH—CH(R^{a3})—C)O)— or a salt thereof. As described herein, R^{a3} is -L^{a}-R′ wherein each variable is independently as described herein. In some embodiments, R′ is R as described herein. In some embodiments, R is an optionally substituted group selected from phenyl, 10-membered bicyclic aryl, 5−6 membered heteroaryl having 1-4 heteroatoms, and 9-10 membered bicyclic heteroaryl having 1-5 heteroatoms. In some embodiments, each substituent is independently halogen or —OH or C₁₋₆ haloaliphatic. In some embodiments, each substituent is independently halogen or —OH. In some embodiments, R is optionally substituted phenyl. In some embodiments, R is phenyl. In some embodiments, R is optionally substituted aryl. In some embodiments, R is aryl. In some embodiments, R is optionally substituted 5-membered heteroaryl having 1-4 heteroatoms. In some embodiments, R is optionally substituted 5-membered heteroaryl having 1 heteroatom. In some embodiments, optionally substituted R is 6-membered heteroaryl having 1-4 heteroatoms. In some embodiments, optionally substituted R is 6-membered heteroaryl having 1 heteroatom. In some embodiments, R is optionally substituted 9-membered heteroaryl having 1-5 heteroatoms. In some embodiments, R is optionally substituted 9-membered heteroaryl having 1 heteroatom. In some embodiments, R is optionally substituted 10-membered heteroaryl having 1-5 heteroatoms. In some embodiments, R is optionally substituted 10-membered heteroaryl having 1 heteroatom. In some embodiments, a heteroatom is nitrogen. In some embodiments, a heteroatom is oxygen. In some embodiments, a heteroatom is sulfur. As described herein, L^{a} is L. In some embodiments, L is a covalent bond. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)ₙ—, wherein n is 1-10. In some embodiments, L is —(CH₂)ₙ—, wherein n is 1-10. In some embodiments, L is —CH₂—. In some embodiments, L is —(CH₂)₂—. In some embodiments, Lis —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—.

In some embodiments, L^{a} is optionally substituted CH₂. In some embodiments, L^{a} is —CH₂—. In some embodiments, X¹³ comprises a side chain which is or comprises an optionally substituted aromatic group, wherein each substituent of the aromatic group is independently selected from halogen, —OR, —R, —N(R)₂, —C(O)N(R)₂, or —CN, wherein each R is independently —H, C₁₋₄ alkyl or haloalkyl, or -Ph. In some embodiments, X¹³ comprises a side chain which is or comprises an optionally substituted aromatic group, wherein each substituent of the aromatic group is independently selected from halogen, —OR, —R, —NH₂, —C(O)NH₂, -Ph, or —CN, wherein each R is independently C₁₋₄ alkyl or haloalkyl. In some embodiments, X¹³ comprises a side chain which is or comprises an optionally substituted aromatic group, wherein each substituent of the aromatic group is independently selected from halogen, —OR, —R, —NH₂, —C(O)NH₂, -Ph, or —CN, wherein each R is independently C₁₋₂ alkyl or haloalkyl. In some embodiments, X¹³ comprises a side chain which is or comprises an optionally substituted aromatic group, wherein each substituent of the aromatic group is independently selected from halogen, —OR, —R, NH₂, —C(O)NH₂, -Ph, or —CN, wherein each R is independently methyl optionally substituted with one or more halogen. In some embodiments, X¹³ comprises a side chain which is or comprises an optionally substituted aromatic group, wherein each substituent of the aromatic group is independently selected from —F, —OR, —CH₃, —NH₂, —C(O)NH₂, -Ph, or —CN, wherein each R is independently methyl optionally substituted with one or more —F. In some embodiments, X¹³ comprises a side chain which is or comprises an optionally substituted aromatic group, wherein each substituent of the aromatic group is independently selected from —F, —OR, —CH₃, —CF₃, —NH₂, —C(O)NH₂, -Ph, or —CN. In some embodiments, X¹³ comprises a side chain which is or comprises an optionally substituted aromatic group, wherein each substituent of the aromatic group is independently selected from halogen, —CH₃, —CF₃, or —CN. In some embodiments, X¹³ comprises a side chain which is or comprises an aromatic group optionally substituted at 2′-position. In some embodiments, X¹³ comprises a side chain which is or comprises an unsubstituted aromatic group. In some embodiments, an aromatic group is optionally substituted 5-membered heteroaryl having 1-3 heteroatoms. In some embodiments, an aromatic group is optionally substituted 5-membered heteroaryl having 1-3 nitrogen atoms. In some embodiments, an aromatic group is optionally substituted 5-membered heteroaryl having one oxygen atom. In some embodiments, an aromatic group is optionally substituted 5-membered heteroaryl having one sulfur atom. In some embodiments, an aromatic group is optionally substituted phenyl. In some embodiments, an aromatic group is optionally substituted 8-10 membered bicyclic aryl or heteroaryl having 1-5 heteroatoms. In some embodiments, an aromatic group is optionally substituted 9-membered bicyclic heteroaryl having 1-5 heteroatoms. In some embodiments, an aromatic group is optionally substituted 10-membered bicyclic heteroaryl having 1-5 heteroatoms. In some embodiments, an aromatic group is optionally substituted 10-membered bicyclic heteroaryl having one heteroatom which is sulfur. In some embodiments, an aromatic group is optionally substituted 10-membered bicyclic heteroaryl having one heteroatom which is oxygen. In some embodiments, an aromatic group is optionally substituted 10-membered bicyclic heteroaryl having one heteroatom which is nitrogen. In some embodiments, an aromatic group is optionally substituted 10-membered bicyclic aryl.

In some embodiments, X¹³ is selected from BztA, Trp, 2NapA, 1NapA, WCHO, 5CpW, 5FW, aMeW, H2Trp, His, Phe, 23FF, 34FF, 340MeF, 1MeW, 5CF3W, 5ClW, 5MeOW, 6ClW, 6F1NapA, 7F1NapA, 7FW, Bip, and Qui. In some embodiments, X¹³ is selected from BztA, Trp, 2NapA, 1NapA, WCHO, 5CpW, 5FW, aMeW, H2Trp, 1MeW, 5CF3W, 5ClW, 5MeOW, 6ClW, 6F1NapA, 7F1NapA, 7FW, and Qui. In some embodiments, X¹³ is selected from BztA, Trp, 2NapA, 1NapA, WCHO, 5CpW, 5FW, aMeW, 1MeW, 5CF3W, 5ClW, 5MeOW, 6ClW, 6F1NapA, 7F1NapA, 7FW, and Qui. In some embodiments, X¹³ is BztA. In some embodiments, X¹³ is Trp. In some embodiments, X¹³ is 1NapA. In some embodiments, X¹³ is 2NapA.

In some embodiments, X¹³ comprises a polar side chain. In some embodiments, X¹³ comprises a non-polar side chain. In some embodiments, X¹³ comprises a hydrophobic side chain. In some embodiments, X¹³ comprises an aliphatic side chain. In some embodiments, X¹³ comprises an alkyl side chain. In some embodiments, X¹³ comprises a side chain comprising a cycloaliphatic group (e.g., a 5- or 6-membered cycloalkyl group). In some embodiments, X¹³ comprises a side chain comprising an optionally substituted aromatic group. In some embodiments, X¹³ comprises a side chain comprising an acidic group, e.g., —COOH. In some embodiments, X¹³ comprises a side chain comprising a basic group, e.g., —N(R)₂. In some embodiments, X¹³ is Gln. In some embodiments, X¹³ is Asn. In some embodiments, X¹³ is Asp. In some embodiments, X¹³ is Glu. In some embodiments, X¹³ is Cha. In some embodiments, X¹³ is CypA. In some embodiments, X¹³ is Ala. In some embodiments, X¹³ is nLeu. In some embodiments, X¹³ is Npg. In some embodiments, X¹³ is [Acryl]Dap.

In some embodiments, X¹³ is selected from BztA, Trp, 2NapA, 1NapA, WCHO, 5CpW, 5FW, Ala, aMeW, H2Trp, His, Phe, 23FF, 34FF, 340MeF, 1MeW, 5CF3W, 5ClW, 5MeOW, 6ClW, 6F1NapA, 7F1NapA, 7FW, Bip, and Qui.

In some embodiments, X¹³ is or comprises a residue of an amino acid or a moiety selected from Table A-IV.

In some embodiments, X¹³ is a residue of 2F3MeW, 34ClF, 34MeF, 3Br4FF, 3BrF, 4ClBztA, 4ClW, 4FW, 5IndA, 7AzaW, 7ClBztA, 7FBztA, Cba, RbMe2NapA, RbMeBztA, SbMe2NapA, SbMeBztA, 1MeW, 1NapA, 23FF, 2F3MeF, 2NapA, 34FF, 340MeF, 3ClF, 3Thi, 4ClF, 5CF3W, 5ClW, 5CpW, 5FW, 5MeOW, 6ClW, 6F1NapA, 7F1NapA, 7FW, Ala, aMeW, Bip, BztA, Cha, H2Trp, His, Phe, PyrS2, Qui, Trp, Tyr, or WCHO.

In some embodiments, X¹³ is a residue of 2F3MeW, 34ClF, 34MeF, 3Br4FF, 3BrF, 4ClBztA, 4ClW, 4FW, 5IndA, 7AzaW, 7ClBztA, 7FBztA, Cba, RbMe2NapA, RbMeBztA, SbMe2NapA, or SbMeBztA.

In some embodiments, X¹³ is a residue of 1MeW, 1NapA, 23FF, 2F3MeF, 2NapA, 34FF, 340MeF, 3ClF, 3Thi, 4ClF, 5CF3W, 5ClW, 5CpW, 5FW, 5MeOW, 6ClW, 6F1NapA, 7F1NapA, 7FW, Ala, aMeW, Bip, BztA, Cha, H2Trp, His, Phe, PyrS2, Qui, Trp, Tyr, or WCHO.

Various types of amino acid residues can be used for X¹⁴, e.g., a residue of an amino acid of formula A-I, A-II, A-III, A-IV, A-V, A-VI, etc. or a salt thereof in accordance with the present disclosure. In some embodiments, X¹⁴ is —N(R^{a1})-L^{a1}-C(R^{a2})(R^{a3})-L^{a2}-C(O)—, wherein each variable is independently as described herein. In some embodiments, X¹⁴ is —N(R^{a1})—C(R^{a2})(R^{a3})—C(O)—, wherein each variable is independently as described herein. In some embodiments, X¹⁴ is —N(R^{a1})—C(R^{a2})H—C(O)—, wherein each variable is independently as described herein. In some embodiments, R^{a1} is —H. In some embodiments, R^{a3} is —H.

In some embodiments, X¹⁴ comprises a polar side chain. In some embodiments, X¹⁴ is a polar amino acid residue as described herein. In some embodiments, X¹⁴ comprises a non-polar side chain. In some embodiments, X¹⁴ comprises a hydrophobic side chain. In some embodiments, X¹⁴ is a hydrophobic amino acid residue as described herein. In some embodiments, X¹⁴ comprises an aliphatic side chain. In some embodiments, X¹⁴ comprises an alkyl side chain. In some embodiments, X¹⁴ comprises a side chain comprising a cycloaliphatic group (e.g., a 5- or 6-membered cycloalkyl group). In some embodiments, X¹⁴ comprises a side chain comprising an optionally substituted aromatic group. In some embodiments, X¹⁴ comprises a side chain comprising an acidic group, e.g., —COOH. In some embodiments, X¹⁴ comprises a side chain comprising a basic group, e.g., —N(R)₂. In some embodiments, X¹⁴ is Gln.

In some embodiments, X¹⁴ is selected from Gln, His, Ser, dThr, Thr, Ala, Hse, Asn, Leu, Aib, Alaol, Throl, Leuol, dAsn, dGln, dHis, Tyr, [AzAc]Lys, 1MeH, 3MeH, 4TriA, dSer, NMeHis, NMeS, Pro, Trp, Val, MorphAla, 2FurA, Abu, Arg, Dab, iPrLys, Phe, Pheol, and Prool.

In some embodiments, X¹⁴ is a residue of an amino alcohol, e.g., Throl, Alaol, Leuol, Pheol or Prool. In some embodiments, an amino alcohol has a structure corresponding an amino acid wherein a —COOH group is replaced with a —OH group. In some embodiments, when X¹⁴ is a residue of an amino alcohol, it is the last residue at the C-terminus. Such a sequence may be properly considered to have —OH as a C-terminus capping group, or such amino alcohol residues may be considered as C-terminus capping groups.

In some embodiments, X¹⁵ is or comprises a residue of an amino acid or a moiety selected from Table A-IV.

In some embodiments, X¹⁴ is or comprises a residue of an amino acid or a moiety selected from Table A-IV.

In some embodiments, X¹⁴ is a residue of [3C]TriAzLys, [4F3CPip]GlnR, [4MePpzPip]GlnR, [4Pippip]GlnR, [AcPpz]GlnR, [bismethoxyethylamine]GlnR, [Me2NPrPip]GlnR, [Morph]GlnR, [NHEt]GlnR, [NMe2]GlnR, [Pip]GlnR, [PropynPEG14]Lys, [RDMAPyr]GlnR, [TfePpz]GlnR, AcLys, BnBoroleK, F2PipNva, GlnR, Me2Asn, Me2Gln, MePpzAsn, Met20, MorphAsn, MorphGln, MorphNva, dAla, MeAsn, 1MeH, 2cbmf, 2F3MeF, 2FurA, 3cbmf, 3MeH, 4TriA, Abu, Aib, Ala, Alaol, Arg, Asn, Asp, BztA, Cha, Dab, dAsn, dGln, dHis, dSer, dThr, Gln, His, Hse, iPrLys, Leu, Leuol, Lys, MorphAla, NMeHis, NMeS, Npg, Phe, Pheol, Pro, Prool, PyrS2, S5, Ser, Thr, Throl, Trp, Tyr, or Val.

In some embodiments, X¹⁴ is a residue of [3C]TriAzLys, [4F3CPip]GlnR, [4MePpzPip]GlnR, [4Pippip]GlnR, [AcPpz]GlnR, [bismethoxyethylamine]GlnR, [Me2NPrPip]GlnR, [Morph]GlnR, [NHEt]GlnR, [NMe2]GlnR, [Pip]GlnR, [PropynPEG14]Lys, [RDMAPyr]GlnR, [TfePpz]GlnR, AcLys, BnBoroleK, F2PipNva, GlnR, Me2Asn, Me2Gln, MePpzAsn, Met20, MorphAsn, MorphGln, MorphNva, dAla, or MeAsn.

In some embodiments, X¹⁴ is a residue of 1MeH, 2cbmf, 2F3MeF, 2FurA, 3cbmf, 3MeH, 4TriA, Abu, Aib, Ala, Alaol, Arg, Asn, Asp, BztA, Cha, Dab, dAsn, dGln, dHis, dSer, dThr, Gln, His, Hse, iPrLys, Leu, Leuol, Lys, MorphAla, NMeHis, NMeS, Npg, Phe, Pheol, Pro, Prool, PyrS2, S5, Ser, Thr, Throl, Trp, Tyr, or Val.

In some embodiments, p14 is 1. In some embodiments, p14 is 0.

Various type of amino acid residues can be utilized for X⁵, e.g., a residue of an amino acid of formula A-I, A-II, A-III, A-IV, A-V, A-VI, etc. or a salt thereof in accordance with the present disclosure. In some embodiments, X⁵ is —N(R^{a1})-L^{a1}-C(R^{a2})(R^{a3})-L^{a2}-C(O)—, wherein each variable is independently as described herein. In some embodiments, X¹⁵ is —N(R^{a1})—C(R^{a2})(R^{a3})—C(O)—, wherein each variable is independently as described herein. In some embodiments, X⁵ is —N(R^{a1})—C(R^{a2})H—C(O)—, wherein each variable is independently as described herein. In some embodiments, R^{a1} is —H. In some embodiments, R^{a3} is —H.

In some embodiments, X¹⁵ comprises a polar side chain as described herein. In some embodiments, X¹⁵ comprises a non-polar side chain. In some embodiments, X¹⁵ comprises a hydrophobic side chain as described herein. In some embodiments, X⁵ comprises an aliphatic side chain. In some embodiments, X¹⁵ comprises an alkyl side chain. In some embodiments, a side chain of X¹⁵ is C₁₋₁₀ alkyl. In some embodiments, X¹⁵ comprises a side chain comprising an optionally substituted aromatic group. In some embodiments, X¹⁵ comprises a side chain comprising an acidic group, e.g., —COOH. In some embodiments, X¹⁵ comprises a side chain comprising a basic group, e.g., —N(R)₂. In some embodiments, X¹⁵ comprises a detectable moiety such as a fluorescent moiety.

In some embodiments, X¹⁵ is Ala. In some embodiments, X¹⁵ is dAla. In some embodiments. In some embodiments, X¹⁵ comprises a detectable moiety such as a fluorescent moiety. In some embodiments, X¹⁵ is Lys. In some embodiments, X¹⁵ is substituted or labeled lysine. In some embodiments, X¹⁵ is selected from [1Napc]Lys, [1NapPr]Lys, [22PhPr]Lys, [2Napc]Lys, [35CF3PhPr]Lys, [4MePipBz]Lys, [AdamantC]Lys, [AzAc]Lys, [Bua]Lys, [Me2NCBz]Lys, [Me3AdamantC]Lys, [MeBipipAc]Lys, [MeMorphBz]Lys, [MePipAc]Lys, [MorphAc]Lys, [mPEG2]Lys, [mPEG4]Lys, [mPEG6]Lys, [mPEG8]Lys, [Oct]Lys, [PropynPEG1]Lys, [PropynPEG2]Lys, [PropynPEG3]Lys, [PropynPEG4]Lys, 6AmHex, 6AzHex, Aib, Ala, dAla, dIle, Ile, and Lys.

In some embodiments, X¹⁵ is a residue of a compound without a carboxyl group, e.g., 6AmHex, 6AzHex, etc. In some embodiments, when X¹⁵ is such a residue, it is the last residue at the C-terminus. Such a sequence may be properly considered to have X¹⁵ as a C-terminus capping group.

In some embodiments, X¹⁵ is or comprises a residue of an amino acid or a moiety selected from Table A-IV.

In some embodiments, X¹⁵ is a residue of [3C]TriAzdLys, [3C]TriAzLys, [lithocholate]-Lys, [lithocholate-PEG2]-Lys, [Me2NCBz]Lys, [Me3AdamantC-PEG2]-Lys, [PropynPEG14]Lys, dAla, dIle, [1Napc]Lys, [1NapPr]Lys, [22PhPr]Lys, [2Napc]Lys, [35CF3PhPr]Lys, [4MePipBz]Lys, [AdamantC]Lys, [Bua]Lys, [Me3AdamantC]Lys, [Me3AdamantC]-Lys, [MeBipipAc]Lys, [MeMorphBz]Lys, [MePipAc]Lys, [MorphAc]Lys, [mPEG2]Lys, [mPEG4]Lys, [mPEG6]Lys, [mPEG8]Lys, [Oct]Lys, [PropynPEG1]Lys, [PropynPEG2]Lys, [PropynPEG3]Lys, [PropynPEG4]Lys, 6AmHex, Aib, Ala, Alaol, BztA, Gln, Ile, Leuol, Leu, Lys, NEt2, NHBn, NHCyHe, NHCyPr, NHEt, OH, Phe, Pheol, Prool, Throl, or Val.

In some embodiments, X¹⁵ is a residue of [3C]TriAzdLys, [3C]TriAzLys, [lithocholate]-Lys, [lithocholate-PEG2]-Lys, [Me2NCBz]Lys, [Me3AdamantC-PEG2]-Lys, [PropynPEG14]Lys, dAla, or dIle.

In some embodiments, X¹⁵ is a residue of [1Napc]Lys, [1NapPr]Lys, [22PhPr]Lys, [2Napc]Lys, [35CF3PhPr]Lys, [4MePipBz]Lys, [AdamantC]Lys, [Bua]Lys, [Me3AdamantC]Lys, [Me3AdamantC]-Lys, [MeBipipAc]Lys, [MeMorphBz]Lys, [MePipAc]Lys, [MorphAc]Lys, [mPEG2]Lys, [mPEG4]Lys, [mPEG6]Lys, [mPEG8]Lys, [Oct]Lys, [PropynPEG1]Lys, [PropynPEG2]Lys, [PropynPEG3]Lys, [PropynPEG4]Lys, 6AmHex, Aib, Ala, Alaol, BztA, Gln, Ile, Leuol, Leu, Lys, NEt2, NHBn, NHCyHe, NHCyPr, NHEt, OH, Phe, Pheol, Prool, Throl, or Val.

In some embodiments, p15 is 1. In some embodiments, p15 is 0.

Various types of amino acid residues can be used for X¹⁶, e.g., a residue of an amino acid of formula A-I, A-II, A-III, A-IV, A-V, A-VI, etc. or a salt thereof in accordance with the present disclosure. In some embodiments, X¹⁶ is —N(R^{a1})-L^{a1}-C(R^{a2})(R^{a3})-L^{a2}-C(O)—, wherein each variable is independently as described herein. In some embodiments, X¹⁶ is —N(R^{a1})—C(R^{a2})(R^{a3})—C(O)—, wherein each variable is independently as described herein. In some embodiments, X¹⁶ is —N(R^{a1})—C(R^{a2})H—C(O)—, wherein each variable is independently as described herein. In some embodiments, R^{a1} is —H. In some embodiments, R^{a3} is —H.

In some embodiments, X¹⁶ comprises a polar side chain. In some embodiments, it is a polar amino acid residue as described herein. In some embodiments, X¹⁶ comprises a non-polar side chain. In some embodiments, X¹⁶ comprises a hydrophobic side chain. In some embodiments, it is a hydrophobic amino acid residue as described herein. In some embodiments, X¹⁶ comprises an aliphatic side chain. In some embodiments, X¹⁶ comprises an alkyl side chain. In some embodiments, a side chain of X¹⁶ is C₁₋₁₀ alkyl. In some embodiments, X¹⁶ comprises a side chain comprising an optionally substituted aromatic group. In some embodiments, it is an aromatic amino acid residue as described herein. In some embodiments, X¹⁶ comprises a side chain comprising an acidic group, e.g., —COOH. In some embodiments, it is an acidic amino acid residue as described herein. In some embodiments, X¹⁶ comprises a side chain comprising a basic group, e.g., —N(R)₂. In some embodiments, it is a basic amino acid residue as described herein. In some embodiments, X¹⁶ comprises a detectable moiety such as a fluorescent moiety. In some embodiments, X¹⁶ is Ala. In some embodiments, X¹⁶ is dAla.

In some embodiments, X¹⁶ is or comprises a residue of an amino acid or a moiety selected from Table A-IV.

In some embodiments, X¹⁶ is a residue of Cbg, Cpg, CyLeu, dLeu, dAla, Aib, Ala, Arg, Asn, dGln, dThr, Gln, Ile, Leu, nLeu, Phe, Ser, Thr, Trp, Tyr, or Val.

In some embodiments, X¹⁶ is a residue of Cbg, Cpg, CyLeu, dLeu, or dAla.

In some embodiments, X¹⁶ is a residue of Aib, Ala, Arg, Asn, dGln, dThr, Gln, Ile, Leu, nLeu, Phe, Ser, Thr, Trp, Tyr, or Val.

In some embodiments, p16 is 1. In some embodiments, p16 is 0.

Various types of amino acid residues can be used for X¹⁷, e.g., a residue of an amino acid of formula A-I, A-II, A-III, A-IV, A-V, A-VI, etc. or a salt thereof in accordance with the present disclosure. In some embodiments, X¹⁷ is —N(R^{a1})-L^{a1}-C(R^{a2})(R^{a3})-L^{a2}-C(O)—, wherein each variable is independently as described herein. In some embodiments, X¹⁷ is —N(R^{a1})—C(R^{a2})(R^{a3})—C(O)—, wherein each variable is independently as described herein. In some embodiments, X¹⁷ is —N(R^{a1})—C(R^{a2})H—C(O)—, wherein each variable is independently as described herein. In some embodiments, R^{a1} is —H. In some embodiments, R^{a3} is —H.

In some embodiments, X¹⁷ comprises a polar side chain. In some embodiments, it is a polar amino acid residue as described herein. In some embodiments, X¹⁷ comprises a non-polar side chain. In some embodiments, X¹⁷ comprises a hydrophobic side chain. In some embodiments, it is a hydrophobic amino acid residue as described herein. In some embodiments, X¹⁷ comprises an aliphatic side chain. In some embodiments, X¹⁷ comprises an alkyl side chain. In some embodiments, a side chain of X¹⁷ is C₁₋₁₀ alkyl. In some embodiments, X¹⁷ comprises a side chain comprising an optionally substituted aromatic group. In some embodiments, it is an aromatic amino acid residue as described herein. In some embodiments, X¹⁷ comprises a side chain comprising an acidic group, e.g., —COOH. In some embodiments, it is an acidic amino acid residue as described herein. In some embodiments, X¹⁷ comprises a side chain comprising a basic group, e.g., —N(R)₂. In some embodiments, it is a basic amino acid residue as described herein. In some embodiments, X¹⁷ comprises a detectable moiety such as a fluorescent moiety. In some embodiments, X¹⁷ is Ala, dAla, or Leu. In some embodiments, X¹⁷ is Ala. In some embodiments, X¹⁷ is dAla. In some embodiments, X¹⁷ is Leu.

In some embodiments, X¹⁷ is or comprises a residue of an amino acid or a moiety selected from Table A-IV.

In some embodiments, p17 is 1. In some embodiments, p17 is 0.

Various types of amino acid residues can be used for X¹⁸, e.g., a residue of an amino acid of formula A-I, A-II, A-III, A-IV, A-V, A-VI, etc. or a salt thereof in accordance with the present disclosure. In some embodiments, X's is —N(R^{a1})-L^{a1}-C(R^{a2})(R^{a3})-L^{a2}-C(O)—, wherein each variable is independently as described herein. In some embodiments, X¹⁸ is —N(R^{a1})—C(R^{a2})(R^{a3})—C(O)—, wherein each variable is independently as described herein. In some embodiments, X's is —N(R^{a1})—C(R^{a2})H—C(O)—, wherein each variable is independently as described herein. In some embodiments, R^{a1} is —H. In some embodiments, R^{a3} is —H.

In some embodiments, X's comprises a polar side chain. In some embodiments, it is a polar amino acid residue as described herein. In some embodiments, X¹⁸ comprises a non-polar side chain. In some embodiments, X¹⁸ comprises a hydrophobic side chain. In some embodiments, it is a hydrophobic amino acid residue as described herein. In some embodiments, X¹⁸ comprises an aliphatic side chain. In some embodiments, X¹⁸ comprises an alkyl side chain. In some embodiments, a side chain of X¹⁸ is C₁₋₁₀ alkyl. In some embodiments, X¹⁸ comprises a side chain comprising an optionally substituted aromatic group. In some embodiments, it is an aromatic amino acid residue as described herein. In some embodiments, X¹⁸ comprises a side chain comprising an acidic group, e.g., —COOH. In some embodiments, it is an acidic amino acid residue as described herein. In some embodiments, X¹⁸ comprises a side chain comprising a basic group, e.g., —N(R)₂. In some embodiments, it is a basic amino acid residue as described herein. In some embodiments, X¹⁸ comprises a detectable moiety such as a fluorescent moiety. In some embodiments, X¹⁸ is Aib, Ala, or Leu. In some embodiments, X¹⁸ is Ala or Leu. In some embodiments, X¹⁸ is Aib. In some embodiments, X¹⁸ is Ala. In some embodiments, X¹⁸ is Leu.

In some embodiments, X¹⁸ is or comprises a residue of an amino acid or a moiety selected from Table A-IV.

In some embodiments, p18 is 1. In some embodiments, p18 is 0.

Various types of amino acid residues can be used for X¹⁹, e.g., a residue of an amino acid of formula A-I, A-II, A-III, A-IV, A-V, A-VI, etc. or a salt thereof in accordance with the present disclosure. In some embodiments, X¹⁹ is —N(R^{a1})-L^{a1}-C(R^{a2})(R^{a3})-L^{a2}-C(O)—, wherein each variable is independently as described herein. In some embodiments, X¹⁹ is —N(R^{a1})—C(R^{a2})(R^{a3})—C(O)—, wherein each variable is independently as described herein. In some embodiments, X¹⁹ is —N(R^{a1})—C(R^{a2})H—C(O)—, wherein each variable is independently as described herein. In some embodiments, R^{a1} is —H. In some embodiments, R^{a3} is —H.

In some embodiments, X¹⁹ comprises a polar side chain. In some embodiments, it is a polar amino acid residue as described herein. In some embodiments, X¹⁹ comprises a non-polar side chain. In some embodiments, X¹⁹ comprises a hydrophobic side chain. In some embodiments, it is a hydrophobic amino acid residue as described herein. In some embodiments, X¹⁹ comprises an aliphatic side chain. In some embodiments, X¹⁹ comprises an alkyl side chain. In some embodiments, a side chain of X¹⁹ is C₁₋₁₀ alkyl. In some embodiments, X¹⁹ comprises a side chain comprising an optionally substituted aromatic group. In some embodiments, it is an aromatic amino acid residue as described herein. In some embodiments, X¹⁹ comprises a side chain comprising an acidic group, e.g., —COOH. In some embodiments, it is an acidic amino acid residue as described herein. In some embodiments, X¹⁹ comprises a side chain comprising a basic group, e.g., —N(R)₂. In some embodiments, it is a basic amino acid residue as described herein. In some embodiments, X¹⁹ comprises a detectable moiety such as a fluorescent moiety. In some embodiments, X¹⁹ is Aib, Ala, or Leu. In some embodiments, X¹⁹ is Ala or Leu. In some embodiments, X¹⁹ is Aib. In some embodiments, X¹⁹ is Ala. In some embodiments, X¹⁹ is Leu.

In some embodiments, X¹⁹ is or comprises a residue of an amino acid or a moiety selected from Table A-IV.

In some embodiments, p19 is 1. In some embodiments, p19 is 0.

Various types of amino acid residues can be used for X²⁰, e.g., a residue of an amino acid of formula A-I, A-II, A-III, A-IV, A-V, A-VI, etc. or a salt thereof in accordance with the present disclosure. In some embodiments, X²⁰ is —N(R^{a1})-L^{a1}-C(R^{a2})(R^{a3})-L^{a2}-C(O)—, wherein each variable is independently as described herein. In some embodiments, X²⁰ is —N(R^{a1})—C(R^{a2})(R^{a3})—C(O)—, wherein each variable is independently as described herein. In some embodiments, X²⁰ is —N(R^{a1})—C(R^{a2})H—C(O)—, wherein each variable is independently as described herein. In some embodiments, R^{a1} is —H. In some embodiments, R^{a3} is —H.

In some embodiments, X²⁰ comprises a polar side chain. In some embodiments, it is a polar amino acid residue as described herein. In some embodiments, X²⁰ comprises a non-polar side chain. In some embodiments, X²⁰ comprises a hydrophobic side chain. In some embodiments, it is a hydrophobic amino acid residue as described herein. In some embodiments, X²⁰ comprises an aliphatic side chain. In some embodiments, X²⁰ comprises an alkyl side chain. In some embodiments, a side chain of X²⁰ is C₁₋₁₀ alkyl. In some embodiments, X²⁰ comprises a side chain comprising an optionally substituted aromatic group. In some embodiments, it is an aromatic amino acid residue as described herein. In some embodiments, X²⁰ comprises a side chain comprising an acidic group, e.g., —COOH. In some embodiments, it is an acidic amino acid residue as described herein. In some embodiments, X²⁰ comprises a side chain comprising a basic group, e.g., —N(R)₂. In some embodiments, it is a basic amino acid residue as described herein. In some embodiments, X²⁰ comprises a detectable moiety such as a fluorescent moiety. In some embodiments, X²⁰ is Aib, Ala, or Leu. In some embodiments, X²⁰ is Ala or Leu. In some embodiments, X²⁰ is Aib. In some embodiments, X²⁰ is Ala. In some embodiments, X²⁰ is Leu.

In some embodiments, X²⁰ is or comprises a residue of an amino acid or a moiety selected from Table A-IV.

In some embodiments, p20 is 1. In some embodiments, p20 is 0.

Various types of amino acid residues can be used for X²¹, e.g., a residue of an amino acid of formula A-I, A-II, A-III, A-IV, A-V, A-VI, etc. or a salt thereof in accordance with the present disclosure. In some embodiments, X²¹ is —N(R^{a1})-L^{a1}-C(R^{a2})(R^{a3})-L^{a2}-C(O)—, wherein each variable is independently as described herein. In some embodiments, X²¹ is —N(R^{a1})—C(R^{a2})(R^{a3})—C(O)—, wherein each variable is independently as described herein. In some embodiments, X²¹ is —N(R^{a1})—C(R^{a2})H—C(O)—, wherein each variable is independently as described herein. In some embodiments, R^{a1} is —H. In some embodiments, R^{a3} is —H.

In some embodiments, X²¹ comprises a polar side chain. In some embodiments, it is a polar amino acid residue as described herein. In some embodiments, X²¹ comprises a non-polar side chain. In some embodiments, X²¹ comprises a hydrophobic side chain. In some embodiments, it is a hydrophobic amino acid residue as described herein. In some embodiments, X²¹ comprises an aliphatic side chain. In some embodiments, X²¹ comprises an alkyl side chain. In some embodiments, a side chain of X²¹ is C₁₋₁₀ alkyl. In some embodiments, X²¹ comprises a side chain comprising an optionally substituted aromatic group. In some embodiments, it is an aromatic amino acid residue as described herein. In some embodiments, X²¹ comprises a side chain comprising an acidic group, e.g., —COOH. In some embodiments, it is an acidic amino acid residue as described herein. In some embodiments, X²¹ comprises a side chain comprising a basic group, e.g., —N(R)₂. In some embodiments, it is a basic amino acid residue as described herein. In some embodiments, X²¹ comprises a detectable moiety such as a fluorescent moiety. In some embodiments, X²¹ is Aib, Ala, or Leu. In some embodiments, X²¹ is Ala or Leu. In some embodiments, X²¹ is Aib. In some embodiments, X²¹ is Ala. In some embodiments, X²¹ is Leu.

In some embodiments, X²¹ is or comprises a residue of an amino acid or a moiety selected from Table A-IV.

In some embodiments, p21 is 1. In some embodiments, p21 is 0.

Various types of amino acid residues can be used for X²², e.g., a residue of an amino acid of formula A-I, A-II, A-III, A-IV, A-V, A-VI, etc. or a salt thereof in accordance with the present disclosure. In some embodiments, X²² is —N(R^{a1})-L^{a1}-C(R^{a2})(R^{a3})-L^{a2}-C(O)—, wherein each variable is independently as described herein. In some embodiments, X²² is —N(R^{a1})—C(R^{a2})(R^{a3})—C(O)—, wherein each variable is independently as described herein. In some embodiments, X²² is —N(R^{a1})—C(R^{a2})H—C(O)—, wherein each variable is independently as described herein. In some embodiments, R^{a1} is —H. In some embodiments, R^{a3} is —H.

In some embodiments, X²² comprises a polar side chain. In some embodiments, it is a polar amino acid residue as described herein. In some embodiments, X²² comprises a non-polar side chain. In some embodiments, X²² comprises a hydrophobic side chain. In some embodiments, it is a hydrophobic amino acid residue as described herein. In some embodiments, X²² comprises an aliphatic side chain. In some embodiments, X²² comprises an alkyl side chain. In some embodiments, a side chain of X²² is C₁₋₁₀ alkyl. In some embodiments, X²² comprises a side chain comprising an optionally substituted aromatic group. In some embodiments, it is an aromatic amino acid residue as described herein. In some embodiments, X²² comprises a side chain comprising an acidic group, e.g., —COOH. In some embodiments, it is an acidic amino acid residue as described herein. In some embodiments, X²² comprises a side chain comprising a basic group, e.g., —N(R)₂. In some embodiments, it is a basic amino acid residue as described herein. In some embodiments, X²² comprises a detectable moiety such as a fluorescent moiety. In some embodiments, X²² is Aib, Ala, or Leu. In some embodiments, X²² is Ala or Leu. In some embodiments, X²² is Aib. In some embodiments, X²² is Ala. In some embodiments, X²² is Leu.

In some embodiments, X²² is or comprises a residue of an amino acid or a moiety selected from Table A-IV.

In some embodiments, p22 is 1. In some embodiments, p22 is 0.

Various types of amino acid residues can be used for X²³, e.g., a residue of an amino acid of formula A-I, A-II, A-III, A-IV, A-V, A-VI, etc. or a salt thereof in accordance with the present disclosure. In some embodiments, X²³ is —N(R^{a1})-L^{a1}-C(R^{a2})(R^{a3})-L^{a2}-C(O)—, wherein each variable is independently as described herein. In some embodiments, X²³ is —N(R^{a1})—C(R^{a2})(R^{a3})—C(O)—, wherein each variable is independently as described herein. In some embodiments, X²³ is —N(R^{a1})—C(R^{a2})H—C(O)—, wherein each variable is independently as described herein. In some embodiments, R^{a1} is —H. In some embodiments, R^{a3} is —H.

In some embodiments, X²³ comprises a polar side chain. In some embodiments, it is a polar amino acid residue as described herein. In some embodiments, X²³ comprises a non-polar side chain. In some embodiments, X²³ comprises a hydrophobic side chain. In some embodiments, it is a hydrophobic amino acid residue as described herein. In some embodiments, X²³ comprises an aliphatic side chain. In some embodiments, X²³ comprises an alkyl side chain. In some embodiments, a side chain of X²³ is C₁₋₁₀ alkyl. In some embodiments, X²³ comprises a side chain comprising an optionally substituted aromatic group. In some embodiments, it is an aromatic amino acid residue as described herein. In some embodiments, X²³ comprises a side chain comprising an acidic group, e.g., —COOH. In some embodiments, it is an acidic amino acid residue as described herein. In some embodiments, X²³ comprises a side chain comprising a basic group, e.g., —N(R)₂. In some embodiments, it is a basic amino acid residue as described herein. In some embodiments, X²³ comprises a detectable moiety such as a fluorescent moiety. In some embodiments, X²³ is Aib, Ala, or Leu. In some embodiments, X²³ is Ala or Leu. In some embodiments, X²³ is Aib. In some embodiments, X²³ is Ala. In some embodiments, X²³ is Leu.

In some embodiments, X²³ is or comprises a residue of an amino acid or a moiety selected from Table A-IV.

In some embodiments, p23 is 1. In some embodiments, p23 is 0.

In some embodiments, an agent is or comprises a peptide having the structure of:
R^{N}—[X]ₚ-X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇—[X]p′-R^{C}, or a salt thereof, wherein:
each X is independently an amino acid residue;
each p and p′ is independently 0-10;
R^{N} is independently a peptide, an amino protecting group or R′-L^{RN}-;
R^{C} is independently a peptide, a carboxyl protecting group, -L^{RC}-R′, —O-L^{RC}-R′ or —N(R′)-L^{RC}-R′;
each of L^{RN} and L^{RC} is independently L; and
each other variable is independently as described herein.

In some embodiments, p is 0. In some embodiments, p is 1, 2, 3, 4, 5, 6, 7, 8, 9, or 10. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10.

In some embodiments, p′ is 0. In some embodiments, p′ is 1, 2, 3, 4, 5, 6, 7, 8, 9, or 10. In some embodiments, p′ is 1. In some embodiments, p′ is 2. In some embodiments, p′ is 3. In some embodiments, p′ is 4. In some embodiments, p′ is 5. In some embodiments, p′ is 6. In some embodiments, p′ is 7. In some embodiments, p′ is 8. In some embodiments, p′ is 9. In some embodiments, p′ is 10.

In some embodiments, each X is independently a residue of an amino acid having the structure of A-I, A-II, A-III, A-IV, etc.

In some embodiments, R^{N} is or comprises a peptide. In some embodiments, R^{N} is a N-terminus capping group. In some embodiments, R^{N} is an amino protecting group. In some embodiments, R^{N} is-L^{RN}-R′. In some embodiments, L^{RN} is —C(O)—. In some embodiments, R^{N} is —C(O)R. In some embodiments, R^{N} is Ac. In some embodiments, R^{N} is AzAc (N₃—CH₂—C(O)—). In some embodiments, R^{N} is 2PyPrpc

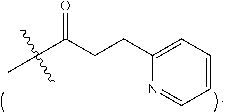

In some embodiments, R^{N} is MeOPr (CH₃OCH₂CH₂C(O)—).

In some embodiments, R^{N} is RSO₂ (—SO₂R). In some embodiments, R^{N} is MeSO₂ (—SO₂CH₃). In some embodiments, R^{N} is mPEG2 (CH₃OCH₂CH₂OCH₂CH₂C(O)—). In some embodiments, wherein R^{N} is Nic

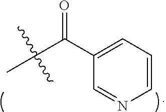

In some embodiments, R^{N} is Oct (CH₃(CH₂)₆C(O)—). In some embodiments, R^{N} is Pic

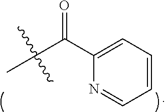

In some embodiments, R^{C} is or comprises a peptide. In some embodiments, R^{C} is a C-terminus capping group. In some embodiments, R^{C} is a carboxyl protecting group. In some embodiments, R^{C} is -L^{RC}-R′. In some embodiments, R^{C} is —O-L^{RC}-R′. In some embodiments, R^{C} is —OR′. In some embodiments, R^{C} is —N(R′)-L^{RC}-R′. In some embodiments, R^{C} is —N(R′)₂. In some embodiments, R^{C} is —NHR′. In some embodiments, R^{C} is —N(R)₂. In some embodiments, R^{C} is —NHR. In some embodiments, R^{C} is —NH₂. In some embodiments, R^{C} is —NHEt. In some embodiments, R^{C} is —NHBn. In some embodiments, R^{C} is —NHCyHe

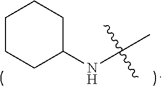

In some embodiments, R^{C} is —NHCyPr

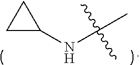

In some embodiments, R^{C} is —NHCyBu. In some embodiments, R^{C} is −6AmHex, wherein one amino group of −6AmHex is bonded to the last —C(O)— of the peptide backbone (R^{C} is —NH—(CH₂)₆—NH₂). In some embodiments, R^{C} is −6AZHex, wherein the amino group of −6AzHex is bonded to the last —C(O)— of the peptide backbone (R^{C} is —NH—(CH₂)₆—N₃). In some embodiments, R^{C} is -Alaol, wherein the amino group of -Alaol is bonded to the last —C(O)— of the peptide backbone (R^{C} is

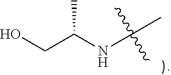 [SMILES: *N[C@@H](C)CO]

In some embodiments, R^{C} is -Leuol, wherein the amino group of -Leuol is bonded to the last —C(O)— of the peptide backbone (R^{C} is

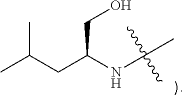 [SMILES: *N[C@H](CO)CC(C)C]

In some embodiments, R^{C} is -Pheol, wherein the amino group of -Pheol is bonded to the last —C(O)— of the peptide backbone (R^{C} is

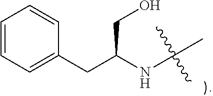 [SMILES: *N[C@H](CO)Cc1ccccc1]

In some embodiments, R^{C} is -Prool, wherein the amino group of -Prool is bonded to the last —C(O)— of the peptide backbone (R^{C} is

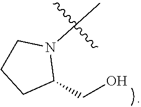 [SMILES: *N1CCC[C@H]1CO]

In some embodiments, R^{C} is -Throl, wherein the amino group of -Throl is bonded to the last —C(O)— of the peptide backbone (R^{C} is

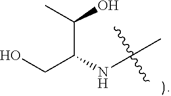 [SMILES: *N[C@H](CO)[C@@H](C)O]

In some embodiments, R^{C} is —OH.

In some embodiments, an agent that binds to beta-catenin comprises an amino acid residue described herein, e.g., a residue of formula AA or a salt form thereof. In some embodiments, an agent that binds to beta-catenin comprises a TfeGA residue. In some embodiments, an agent that binds to beta-catenin comprises a 2COOHF residue. In some embodiments, an agent that binds to beta-catenin comprises a 3COOHF residue. In some embodiments, such a residue is X², X⁵ or X⁶. In some embodiments, such a residue is X⁵. In some embodiments, such a residue is X⁶.

Certain useful agents (e.g., stapled peptides) that bind to beta-catenin and compositions thereof are presented in Table E3 as examples; certain data are presented in Table E2 as examples.

### Amino Acids

As appreciated by those skilled in the art, various amino acids may be utilized in accordance with the present disclosure. For example, both naturally occurring and non-naturally occurring amino acids can be utilized in accordance with the present disclosure. In some embodiments, an amino acid is a compound comprising an amino group that can form an amide group with a carboxyl group and a carboxyl group. In some embodiments, an amino acid is an alpha amino acid. In some embodiments, an amino acid is a beta-amino acid. In some embodiments, an amino acid is a D-amino acid. In some embodiments, an amino acid is a L-amino acid. In some embodiments, an amino acid is an naturally encoded amino acid, e.g., in mammalian cells.

In some embodiments, an amino acid is a compound having the structure of formula A-I:

NH(R^{a1})-L^{a1}-C(R^{a2})(R^{a3})-L^{a2}-COOH, A-I

or a salt thereof, wherein:
each of R^{a1}, R^{a2}, R^{a3} is independently -L^{a}-R′;
each of La, L^{a1} and L^{a2} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a ring moiety of, e.g., -Cy-, R (including those formed by R groups taken together), etc. is monocyclic. In some embodiments, a ring moiety is bicyclic or polycyclic. In some embodiments, a monocyclic ring is an optionally substituted 3-10 (3, 4, 5, 6, 7, 8, 9, or 10, 3-8, 3-7, 4-7, 4−6, 5−6, etc.) membered, saturated, partially unsaturated or aromatic ring having 0-5 heteroatoms. In some embodiments, each monocyclic ring unit of a bicyclic or polycyclic ring moiety is independently an optionally substituted 3-10 (3, 4, 5, 6, 7, 8, 9, or 10, 3-8, 3-7, 4-7, 4−6, 5−6, etc.) membered, saturated, partially unsaturated or aromatic ring having 0-5 heteroatoms.

In some embodiments, each heteroatom is independently selected from oxygen, nitrogen, sulfur, phosphorus and silicon. In some embodiments, each heteroatom is independently selected from oxygen, nitrogen, and sulfur.

In some embodiments, L^{a1} is a covalent bond. In some embodiments, a compound of formula A-1 is of the structure NH(R^{a1})—C(R^{a2})(R^{a3})-L^{a2}-COOH.

In some embodiments, L^{a2} is a covalent bond. In some embodiments, a compound of formula A-1 is of the structure NH(R^{a1})—C(R^{a2})(R^{a3})-L^{a2}-COOH.

In some embodiments, L^{a1} is a covalent bond and L^{a2} is a covalent bond. In some embodiments, a compound of formula A-1 is of the structure NH(R^{a1})—C(R^{a2})(R^{a3})—COOH.

In some embodiments, an amino acid is suitable for stapling. In some embodiments, an amino acid comprises a terminal olefin. Certain such amino acids are exemplified herein (e.g., those described in or utilized in peptides of various Tables).

In some embodiments, an agent comprises a detectable moiety, which can either be detected directly or indirectly. For example, in some embodiments, a detectable moiety is or comprises a fluorescent group. In some embodiments, a detectable moiety is or comprises a biotin moiety. In some embodiments, a detectable moiety is connected to the rest of an agent at an amino acid residue, e.g., through a side chain, optionally through a linker (e.g., L as described herein). In some embodiments, a detectable moiety is —N₃, which may be detected after a click chemistry reaction with a labeled agent comprising an alkyne.

In some embodiments, the present disclosure provides various compounds, which among other things may be utilized as amino acids for a number of applications, e.g., for preparation of peptides or other useful compounds.

In some embodiments, a compound (e.g., an amino acid or a protected and/or activated form thereof) or a salt thereof comprises 1) a first group which is an optionally protected amino group, 2) a second group which is an optionally protected and/or activated carboxyl group, and 3) a side chain (typically bonded to an atom between the first and second groups (“a side chain attachment atom”)) which comprises an optionally protected and/or activated carboxyl group and a) an optionally substituted ring (which ring is typically between the optionally protected and/or activated carboxyl group of the side chain and a side chain attachment atom) or b) an amino group (which amino group is typically between the optionally protected and/or activated carboxyl group of the side chain and a side chain attachment atom). In some embodiments, a provided compound is an optionally protected and/or activated amino acid or a salt thereof, wherein the side chain of the amino acid comprises an optionally protected and/or activated carboxyl group, and an optionally substituted ring or an amino group, wherein the optionally substituted ring or an amino group is between the optionally protected and/or activated carboxyl group and a backbone atom to which a side chain is attached (e.g., an atom between an amino and carboxyl group, both of which can be optionally and independently protected and/or activated (e.g., an alpha carbon atom in an amino acid)).

In some embodiments, the present disclosure provides compounds having the structure of formula PA:

N(R^{PA})(R^{a1})-L^{a1}-C(R^{a2})(R^{a3})-L^{a2}-C(O)R^{PC} PA

or a salt thereof, wherein:
R^{PA} is —H or an amino protecting group;
each of R^{a1} and R^{a3} is independently -L^{a}-R′;
R^{a2} is -L^{aa}-C(O)R^{PS};
each of L^{a}, L^{a1} and L^{a2} is independently L;
—C(O)R^{PS} is optionally protected or activated —COOH;
—C(O)R^{C} is optionally protected or activated —COOH;
each L is independently a covalent bond, or an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently —R, —C(O)R, —CO₂R, or —SO₂R; and
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, compounds (e.g., amino acids, such as those of formula A-I or protected/activated forms thereof) having the structure of formula PA:

N(R^{PA})(R^{a1})-L^{a1}-C(R^{a2})(R^{a3})-L^{a2}-C(O)R^{PC} PA

or a salt thereof, wherein:
R^{PA} is —H or an amino protecting group;
each of R^{a1} and R^{a3} is independently -L^{a}-R′;
R^{a2} is -L^{aa}-C(O)R^{PS}, wherein L^{aa} is L and L^{aa} comprises —N(R′)— or -Cy-;
each of L^{a1} and L^{a2} is independently L;
—C(O)R^{PS} is optionally protected or activated —COOH;
—C(O)R^{C} is optionally protected or activated —COOH;
each L is independently a covalent bond, or an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently —R, —C(O)R, —CO₂R, or —SO₂R; and
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, L^{a1} is a covalent bond. In some embodiments, L^{a1} is not a covalent bond.

In some embodiments, L^{a2} is a covalent bond. In some embodiments, L^{a2} is not a covalent bond.

In some embodiments, R^{a2} is -L^{aa}-C(O)R^{PS}, wherein L^{aa} is an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein at least one methylene unit is replaced with -Cy-.

As used herein, in some embodiments, -Cy- is an optionally substituted bivalent 3-10 (e.g., 3, 4, 5, 6, 7, 8, 9, or 10) membered monocyclic cycloaliphatic group. In some embodiments, -Cy- is an optionally substituted 3-10 (e.g., 3, 4, 5, 6, 7, 8, 9, or 10) membered monocyclic cycloalkyl ring. In some embodiments, -Cy- is an optionally substituted 3-10 (e.g., 3, 4, 5, 6, 7, 8, 9, or 10) membered monocyclic heteroaliphatic ring having 1-5 heteroatoms. In some embodiments, -Cy- is an optionally substituted 3-10 (e.g., 3, 4, 5, 6, 7, 8, 9, or 10) membered monocyclic heteroalkyl ring having 1-5 heteroatoms. In some embodiments, -Cy- is an optionally substituted bivalent 5-15 (e.g., 5, 6, 7, 8, 9, 10, 11, 12, 13, 14, or 15) membered bicyclic or polycyclic cycloaliphatic group. In some embodiments, -Cy- is an optionally substituted bivalent 5-15 (e.g., 5, 6, 7, 8, 9, 10, 11, 12, 13, 14, or 15) membered bicyclic or polycyclic cycloalkyl group. In some embodiments, -Cy- is an optionally substituted 5-15 (e.g., 5, 6, 7, 8, 9, 10, 11, 12, 13, 14, or 15) membered bicyclic or polycyclic heteroaliphatic ring having 1-5 heteroatoms. In some embodiments, -Cy- is an optionally substituted 5-15 (e.g., 5, 6, 7, 8, 9, 10, 11, 12, 13, 14, or 15) membered bicyclic or polycyclic heterocyclyl ring having 1-5 heteroatoms. In some embodiments, a cycloaliphatic, cycloalkyl, heteroaliphatic or heteroalkyl ring is 3-membered. In some embodiments, it is 4-membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, it is 11-membered. In some embodiments, it is 12-membered. In some embodiments, -Cy- is optionally substituted phenylene. In some embodiments, -Cy- is an optionally substituted bivalent 10-membered bicyclic aryl ring. In some embodiments, -Cy- is an optionally substituted 5-membered heteroaryl ring having 1-4 heteroatoms. In some embodiments, -Cy- is an optionally substituted 6-membered heteroaryl ring having 1-4 heteroatoms. In some embodiments, -Cy- is an optionally substituted 9-membered bicyclic heteroaryl ring having 1-5 heteroatoms. In some embodiments, -Cy- is an optionally substituted 10-membered bicyclic heteroaryl ring having 1-5 heteroatoms. In some embodiments, a heteroaliphatic, heterocyclyl or heteroaryl ring contains no more than 1 heteroatom. In some embodiments, each heteroatom is independently selected from nitrogen, oxygen and sulfur.

In some embodiments, -Cy- is an optionally substituted 4-7 membered ring having 0-3 heteroatoms. In some embodiments, -Cy- is an optionally substituted 6-membered aryl ring. In some embodiments, an aryl ring is substituted. In some embodiments, it is substituted with one or more halogen. In some embodiments, it is substituted with one or more —F. In some embodiments, it is not substituted. In some embodiments, it is optionally substituted

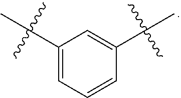 [SMILES: *c1cccc(*)c1]

In some embodiments, it is

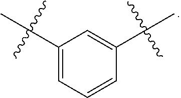 [SMILES: *c1cccc(*)c1]

In some embodiments, it is optionally substituted

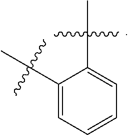 [SMILES: *c1ccccc1*]

In some embodiments, it is

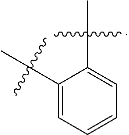 [SMILES: *c1ccccc1*]

In some embodiments, it is optionally substituted

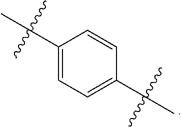 [SMILES: *c1ccc(*)cc1]

In some embodiments, it is

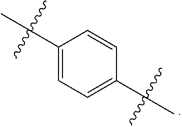 [SMILES: *c1ccc(*)cc1]

In some embodiments, -Cy- is an optionally substituted 5-membered heteroaryl ring having 1-3 heteroatoms. In some embodiments, a heteroatom is nitrogen. In some embodiments, a heteroatom is oxygen. In some embodiments, a heteroatom is sulfur. In some embodiments, -Cy- is optionally substituted

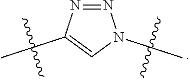 [SMILES: *c1cn(*)nn1]

In some embodiments, -Cy- is

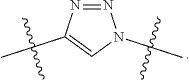 [SMILES: *c1cn(*)nn1]

In some embodiments, L^{aa} is -L^{am1}-Cy-L^{am2}-, wherein each of L^{am1} and L^{am2} is independently L^{am}, wherein each L^{am} is independently a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, L^{aa} comprises -Cy-. In some embodiments, L^{aa} is -L^{am1}-Cy-L^{am2}-, wherein each of L^{am1}and L^{am2} is independently L^{am}, wherein each L^{am} is independently a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, -L^{am2}- is bonded to —C(O)R^{PS}. In some embodiments, L^{am2} is a covalent bond.

In some embodiments, -Cy- is an optionally substituted 4-7 membered ring having 0-3 heteroatoms. In some embodiments, -Cy- is an optionally substituted 5-7 membered ring having 0-3 heteroatoms. In some embodiments, -Cy- is an optionally substituted 6-7 membered ring having 0-3 heteroatoms. In some embodiments, -Cy- is an optionally substituted 4-membered ring having 0-1 heteroatoms. In some embodiments, -Cy- is an optionally substituted 5-membered ring having 0-2 heteroatoms. In some embodiments, -Cy- is an optionally substituted 6-membered ring having 0-2 heteroatoms. In some embodiments, -Cy- is an optionally substituted 7-membered ring having 0-3 heteroatoms.

In some embodiments, R^{a2} is -L^{aa}-C(O)R^{PS}, wherein L^{aa} is an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein at least one methylene unit is replaced with —N(R′)—.

In some embodiments, L^{aa} comprises —N(R′)—. In some embodiments, L^{aa} is -L^{am1}-(NR′)-L^{am2}-, wherein each of L^{am1} and L^{am2} is independently L^{am}, wherein each L^{am} is independently a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, -L^{am2}- is bonded to —C(O)R^{PS}. In some embodiments, L^{am1} is optionally substituted C₁₋₄ alkylene. In some embodiments, L^{am1} is optionally substituted —(CH₂)m-, wherein m is 1, 2, 3, or 4. In some embodiments, L^{am1} is —CH₂—. In some embodiments, L^{am2} is optionally substituted linear C₁₋₂ alkylene. In some embodiments, L^{am2} is —[C(R′)₂]n, wherein n is 1 or 2. In some embodiments, L^{am2} is —[CHR′]n, wherein n is 1 or 2. In some embodiments, each R′ is independently —H or optionally substituted C₁₋₆ alkyl. In some embodiments, L^{am2} is optionally substituted —CH₂—. In some embodiments, L^{am2} is —CH₂—. In some embodiments, R′ is —R^{NR}, wherein R^{NR} is R. In some embodiments, R′ is —CH₂—R^{NR}, wherein R^{NR} is R. In some embodiments, R′ of the —N(R′)— is —C(O)R^{NR}, wherein R^{NR} is R. In some embodiments, R′ of the —N(R′)— is —SO₂RN, wherein R^{NR} is R. In some embodiments, R is optionally substituted C₁₋₆ aliphatic or heteroaliphatic having 1-4 heteroatoms. In some embodiments, R is C₁₋₇ alkyl or heteroalkyl having 1-4 heteroatoms optionally substituted with one or more groups independently selected from halogen, a C₅₋₆ aromatic ring having 0-4 heteroatoms, and an optionally substituted 3-10 membered cycloalkyl or heteroalkyl ring having 1-4 heteroatoms. In some embodiments, R is —CF₃. In some embodiments, L^{am2} is or comprises —C(R′)₂— wherein the R′ group and R′ in —N(R′)— are taken together with their intervening atoms to form an optionally substituted, 3-30 membered, monocyclic, bicyclic or polycyclic ring having, in addition to the intervening atoms, 0-10 heteroatoms.

In some embodiments, L^{aa} is -L^{am1}-N(R′)-L^{am2}-, wherein each of L^{am1} and L^{am2} is independently L^{am}, wherein each L^{am} is independently a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, —N(R′)— is bonded to two carbon atoms which two carbon atoms do not form any double bonds with heteroatoms. In some embodiments, —N(R′)— is bonded to two sp3 atoms. In some embodiments, —N(R′)— is bonded to two sp3 carbon atoms. In some embodiments, —N(R′)— is bonded to two —CH₂—, each of which is independently and optionally substituted with one or two monovalent substituent. In some embodiments, —N(R′)— is bonded to two —CH₂—.

In some embodiments, L^{aa} comprises —N(R′)—. In some embodiments, R′ of the —N(R′)— is —R^{NR}, wherein R^{NR} is R. In some embodiments, R′ of the —N(R′)— is —CH₂—R^{N}, wherein R^{NR} is R, and the —CH₂— is optionally substituted. In some embodiments, R′ of the —N(R′)— is —C(O)R^{NR}, wherein R^{NR} is R. In some embodiments, R′ of the —N(R′)— is —SO₂R^{NR}, wherein R^{NR} is R. In some embodiments, —N(R′)— is —N(Et)-. In some embodiments, —N(R′)— is —N(CH₂CF₃)—. In some embodiments, R′ is optionally substituted C₁₋₆ aliphatic or heteroaliphatic having 1-4 heteroatoms. In some embodiments, R′ is C₁₋₇ alkyl or heteroalkyl having 1-4 heteroatoms, wherein the alkyl or heteroalkyl is optionally substituted with one or more groups independently selected from halogen, a C₅₋₆ aromatic ring having 0-4 heteroatoms, and an optionally substituted 3-10 membered cycloalkyl or heteroalkyl ring having 1-4 heteroatoms. In some embodiments, R is —CF₃.

In some embodiments, R′ of —N(R′)— is R, R^{a3} is R, and the two R groups are taken together with their intervening atoms to form an optionally substituted 3-10 membered ring having 0-5 heteroatoms in addition to the intervening atoms. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is monocyclic. In some embodiments, a formed ring is bicyclic or polycyclic. In some embodiments, a formed ring is saturated. In some embodiments, a formed ring is partially unsaturated.

In some embodiments, L^{am1} is a covalent bond. In some embodiments, L^{am1} is not a covalent bond. In some embodiments, L^{am1} is optionally substituted C₁₋₄ alkylene. In some embodiments, L^{am1} is optionally substituted —(CH₂)m-, wherein m is 1, 2, 3, or 4. In some embodiments, L^{am1} is optionally substituted —CH₂—. In some embodiments, L^{a}m is —CH₂—.

In some embodiments, L^{am2} is bonded to —C(O)R^{PS}.

In some embodiments, L^{am2} is a covalent bond. In some embodiments, L^{am2} is a covalent bond when it is between -Cy- and —C(O)R^{PS}. In some embodiments, L^{am2} is not a covalent bond. In some embodiments, L^{am2} is optionally substituted C₁₋₄ alkylene. In some embodiments, L^{am2} is optionally substituted —(CH₂)m-, wherein m is 1, 2, 3, or 4. In some embodiments, L^{am2} is optionally substituted linear C₁₋₂ alkylene. In some embodiments, L^{ai2} is -[C(R′)₂]n, wherein n is 1 or 2. In some embodiments, L^{am2} is —[CHR′]n, wherein n is 1 or 2. In some embodiments, each R′ is independently —H or optionally substituted C₁₋₆ alkyl. In some embodiments, L^{am2} is optionally substituted —CH₂—. In some embodiments, L^{am2} is —CH₂—. In some embodiments, L^{am2} is optionally substituted —CH₂—CH₂—. In some embodiments, L^{am2} is —CH₂—C(CH₃)₂—.

In some embodiments, L^{am2} is or comprises —C(R′)₂— wherein the R′ group and R′ in —N(R′)— of L^{aa} are taken together with their intervening atoms to form an optionally substituted, 3-30 membered, monocyclic, bicyclic or polycyclic ring having, in addition to the intervening atoms, 0-10 heteroatoms.

In some embodiments, R^{a2} is -L^{aa}-C(O)R^{PS}, wherein L^{aa} is L as described herein. In some embodiments, L^{aa} is L^{an}a as described herein. In some embodiments, L^{aa} is optionally substituted branched or linear C₁₋₁₀ hydrocarbon chain. In some embodiments, L^{aa} is optionally substituted C₁₋₁₀ (e.g., 1, 2, 3, 4, 5, 6, 7, 8, 9, or 10) alkylene. In some embodiments, L^{aa} is optionally substituted —CH₂—CH₂—. In some embodiments, L^{aa} is —CH₂—CH₂—. In some embodiments, L^{aa} is optionally substituted —CH₂—. In some embodiments, L^{aa} is —CH₂—.

In some embodiments, L^{a} is L^{aa} as described herein.

In some embodiments, L^{aa} is L^{a} as described herein.

As described above, each L is independently a covalent bond, or an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, L is a covalent bond.

In some embodiments, L (or L^{a}, L^{aa}, L^{a1}, L^{a2}, L^{s1}, L^{s2}, L^{s3}, or another variable or moiety that can be L, or a linker moiety) is an optionally substituted, bivalent C₁-C₂₅, C₁-C₂₀, C₁-C₁₅, C₁-C₁₀, C₁-C₉, C₁-C₅, C₁-C₇, C₁-C₆, C₁-C₅, C₁-C₄, C₁-C₃, C₁-C₂, or C₁, C₂, C₃, C₄, C₅, C₆, C₇, C₈, C₉, C₁₀, C₁, C₁₂, C₁₃, C₁₄, C₁₅, C₁₆, C₁₇, C₁₈, C₁₉, or C₂₀, aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L is an optionally substituted, bivalent C₁-C₂₅ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, L, L^{a}, L^{aa}, L^{a1}, L^{a2}, L^{s1}, L^{s2}, L^{s3}, L″, or another variable or moiety that can be L, or a linker moiety, is an optionally substituted, bivalent C₁-C₂₅, C₁-C₂₀, C₁-C₁₅, C₁-C₁₀, C₁-C₉, C₁-C₅, C₁-C₇, C₁-C₆, C₁-C₅, C₁-C₄, C₁-C₃, C₁-C₂, or C₁, C₂, C₃, C₄, C₅, C₆, C₇, C₈, C₉, C₁₀, C₁, C₁₂, C₁₃, C₁₄, C₁₅, C₁₆, C₁₇, C₁₈, C₁₉, or C₂₀, aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₁-C₁₀, C₁-C₉, C₁-C₅, C₁-C₇, C₁-C₆, C₁-C₅, C₁-C₄, C₁-C₃, C₁-C₂, or C₁, C₂, C₃, C₄, C₅, C₆, C₇, C₈, C₉, or C₁₀, aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₂ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₃ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₄ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₅ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₆ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the bivalent aliphatic is saturated. In some embodiments, the bivalent aliphatic is linear. In some embodiments, the bivalent aliphatic is branched. In some embodiments, it is an optionally substituted, bivalent linear saturated C₆ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, each replacement if any is independently with -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, each replacement if any is independently with -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, each replacement if any is independently with —O—, —S—, —N(R′)—, —C(O)—, —S(O)—, —S(O)₂—, —S(O)₂N(R′)—, —C(O)S—, or —C(O)O—. In some embodiments, each replacement if any is independently with —O—, —S—, —N(R′)—, or —C(O)—. In some embodiments, L, L^{a}, L^{aa}, L^{a1}, L^{a2}, L^{s1}, L^{s2}, L^{s3}, L″, or another variable or moiety that can be L, or a linker moiety, is an optionally substituted, bivalent C₁-C₆ linear saturated aliphatic wherein one or more methylene units is optionally and independently replaced with —O—, —S—, —N(R′)—, or —C(O)—. In some embodiments, it is an optionally substituted, bivalent C₁-C₅ linear saturated aliphatic wherein one or more methylene units is optionally and independently replaced with —O—, —S—, —N(R′)—, or —C(O)—. In some embodiments, it is an optionally substituted, bivalent C₁-C₄ linear saturated aliphatic wherein one or more methylene units is optionally and independently replaced with —O—, —S—, —N(R′)—, or —C(O)—. In some embodiments, it is an optionally substituted, bivalent C₁-C₃ linear saturated aliphatic wherein one or more methylene units is optionally and independently replaced with —O—, —S—, —N(R′)—, or —C(O)—. In some embodiments, it is an optionally substituted, bivalent C₁-C₂ linear saturated aliphatic wherein one or more methylene units is optionally and independently replaced with —O—, —S—, —N(R′)—, or —C(O)—. In some embodiments, it is a bivalent C₁-C₆ linear saturated aliphatic wherein one or more methylene units is optionally and independently replaced with —O—, —S—, —N(R′)—, or —C(O)—. In some embodiments, it is a bivalent C₁-C₅ linear saturated aliphatic wherein one or more methylene units is optionally and independently replaced with —O—, —S—, —N(R′)—, or —C(O)—. In some embodiments, it is a bivalent C₁-C₄ linear saturated aliphatic wherein one or more methylene units is optionally and independently replaced with —O—, —S—, —N(R′)—, or —C(O)—. In some embodiments, it is a bivalent C₁-C₃ linear saturated aliphatic wherein one or more methylene units is optionally and independently replaced with —O—, —S—, —N(R′)—, or —C(O)—. In some embodiments, it is a bivalent C₁-C₂ linear saturated aliphatic wherein one or more methylene units is optionally and independently replaced with —O—, —S—, —N(R′)—, or —C(O)—. In some embodiments, there is no replacement of methylene unit. In some embodiments, there is one replacement. In some embodiments, there is two replacement. In some embodiments, there is three replacement. In some embodiments, there is four or more replacement. In some embodiments, R′ in each moiety that is utilized to replace a methylene unit (e.g., —N(R′)—) as described herein is hydrogen or optionally substituted C₁−6 aliphatic or phenyl. In some embodiments, R′ is each such moiety is hydrogen or optionally substituted C₁₋₆ alkyl. In some embodiments, R′ is each such moiety is hydrogen or C₁₋₆ alkyl. In some embodiments, each -Cy- is optionally substituted bivalent ring selected from 3-10, 3-9, 3-8, 3-7, 5-10, 5-9, 5-8, 5-7, 5−6, or 3, 4, 5, 6, 7, 8, 9, or 10 membered cycloaliphatic and heterocyclylene having 1-3 heteroatoms, phenylene, and 5−6 membered heteroarylene having 1-3 heteroatoms. In some embodiments, -Cy- is optionally substituted bivalent 3-10, 3-9, 3-8, 3-7, 5-10, 5-9, 5-8, 5-7, 5−6, or 3, 4, 5, 6, 7, 8, 9, or 10 membered cycloaliphatic. In some embodiments, -Cy- is optionally substituted 3-10, 3-9, 3-8, 3-7, 5-10, 5-9, 5-8, 5-7, 5−6, or 3, 4, 5, 6, 7, 8, 9, or 10 membered heterocyclylene having 1-3 heteroatoms. In some embodiments, -Cy- is optionally substituted 3-10, 3-9, 3-8, 3-7, 5-10, 5-9, 5-8, 5-7, 5−6, or 3, 4, 5, 6, 7, 8, 9, or 10 membered heterocyclylene having 1 heteroatom. In some embodiments, -Cy- is optionally substituted phenylene. In some embodiments, -Cy- is phenylene. In some embodiments, -Cy- is optionally substituted 5−6 membered heteroarylene having 1-3 heteroatoms. In some embodiments, -Cy- is optionally substituted 5−6 membered heteroarylene having 1 heteroatom. In some embodiments, a heteroatom is nitrogen. In some embodiments, a heteroatom is oxygen. In some embodiments, a heteroatom is sulfur. In some embodiments, L, L^{a}, L^{aa}, L^{a1}, L^{a2}, L^{s1}, L^{s2}, L^{s3}, L″, or another variable or moiety that can be L, or a linker moiety, is optionally substituted —(CH₂)ₙ—. In some embodiments, it is —(CH₂)ₙ—. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, L, L^{a}, L^{aa}, L^{a1}, L^{a2}, L^{s1}, L^{s2}, L^{s3}, L″, or another variable or moiety that can be L, or a linker moiety, is an optionally substituted, bivalent heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

Those skilled in the art appreciate that embodiments described for one linker moiety that can be L or L″ (e.g., L^{aa}, L^{s1}, L^{s2}, L^{s3}, L^{s}, L^{a}, L^{a1}, L^{a2}, L^{RN}, etc.) may also be utilized for another group that can be L or L″ to the extent that such embodiments fall within the definition of L or L″.

As described above, each R′ is independently —R, —C(O)R, —CO₂R, or —SO₂R. In some embodiments, R′ is -L^{a}-R. In some embodiments, R′ is R. In some embodiments, R′ is —C(O)R. In some embodiments, R′ is —CO₂R. In some embodiments, R′ is —SO₂R. In some embodiments, R′ is —H.

As described above, each R is independently —H, or an optionally substituted group selected from C₁₋₃₀ aliphatic, C₁-30 heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

As described herein, in some embodiments, R is —H. In some embodiments, R is not —H. In some embodiments, R is optionally substituted C₁₋₁₀ aliphatic. In some embodiments, R is optionally substituted C₁₋₁₀ alkyl. In some embodiments, R is methyl. In some embodiments, R is ethyl. In some embodiments, R is isopropyl. In some embodiments, R is —CF₃. In some embodiments, R is —CH₂CF₃. In some embodiments, R is butyl. In some embodiments, R is t-butyl. In some embodiments, R is optionally substituted C₃₋₁₀ cycloaliphatic. In some embodiments, R is optionally substituted C₃₋₁₀ cycloalkyl. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted phenyl. In some embodiments, R is phenyl. In some embodiments, R is optionally substituted 5-membered heteroaryl having 1-3 heteroatoms. In some embodiments, R is optionally substituted 5-membered heteroaryl having 1 heteroatom. In some embodiments, R is optionally substituted 6-membered heteroaryl having 1-3 heteroatoms. In some embodiments, R is optionally substituted 6-membered heteroaryl having 1 heteroatom. In some embodiments, R is optionally substituted bicyclic 8-10 membered aromatic ring having 0-5 heteroatoms. In some embodiments, R is optionally substituted bicyclic 9-membered aromatic ring having 1-5 heteroatoms. In some embodiments, R is optionally substituted bicyclic 10-membered aromatic ring having 1-5 heteroatoms. In some embodiments, R is optionally substituted bicyclic 9-membered aromatic ring having 1 heteroatom. In some embodiments, R is optionally substituted bicyclic 10-membered aromatic ring having 1 heteroatom. In some embodiments, R is optionally substituted bicyclic 10-membered aromatic ring having no heteroatom. In some embodiments, R is optionally substituted 3-10 membered heterocyclyl having 1-5 heteroatoms. In some embodiments, R is optionally substituted 5-14 membered bicyclic heterocyclyl having 1-5 heteroatoms.

In some embodiments, two R groups (or two groups that can be R, e.g., two groups each independently selected from R′, R^{a1}, R^{a2}, R^{a3}, R^{a5}, R^{RN}, etc.) are taken together with their intervening atom(s) to form an optionally substituted 3-30 membered, monocyclic, bicyclic or polycyclic ring having, in addition to the atom, 0-10 heteroatoms. In some embodiments, a formed ring is substituted. In some embodiments, a formed ring is unsubstituted. In some embodiments, a formed ring is 3-30, 3-20, 3-15, 3-10, 3-9, 3-8, 3-7, 3−6, 4-10, 4-9, 4-8, 4-7, 4−6, 5-10, 5-9, 5-8, 5-7, 5−6, or 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 membered. In some embodiments, a formed ring is 3-10 membered. In some embodiments, a formed ring is 3-7 membered. In some embodiments, a formed ring is 4-10 membered. In some embodiments, a formed ring is 4-7 membered. In some embodiments, a formed ring is 5-10 membered. In some embodiments, a formed ring is 5-7 membered. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring is 9-membered. In some embodiments, a formed ring is 10-membered. In some embodiments, a formed ring is monocyclic. In some embodiments, a formed ring is bicyclic. In some embodiments, a formed ring is polycyclic. In some embodiments, a formed ring has no heteroatoms in addition to the intervening atom(s). In some embodiments, a formed ring has 1-10, e.g., 1-5, 1-3, or 1, 2, 3, 4, 5, 6, 7, 8, 9, or 10 heteroatoms in addition to the intervening atom(s). In some embodiments, a formed ring is saturated. In some embodiments, a formed ring is partially unsaturated. In some embodiments, a formed ring comprises one or more aromatic ring. In some embodiments, a formed ring is bicyclic or polycyclic, and each monocyclic unit is independently 3-10 membered, saturated, partially unsaturated or aromatic and having 0-5 heteroatoms. In some embodiments, each heteroatom is independently selected from nitrogen, oxygen and sulfur.

In some embodiments, a group that can be R, e.g., R′, R^{a1}, R^{a2}, R^{a3}, R^{a5}, R^{RN}, etc., is R as described herein. Those skilled in the art appreciate that embodiments described for one group that can be R may also be utilized for another group that can be R to the extent that such embodiments fall within the definition of R.

In some embodiments, the present disclosure provides compounds having the structure of

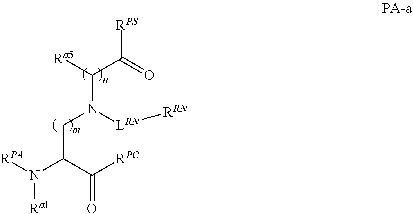

or a salt thereof, wherein:
each of m and n is independently 1, 2, 3, or 4;
L^{RN} is L;
R^{RN} is R; and
R^{a5} is R′.

In some embodiments, m is 1. In some embodiments, m is 2. In some embodiments, m is 3. In some embodiments, m is 4.

In some embodiments, n is 1. In some embodiments, n is 2. In some embodiments, n is 3. In some embodiments, n is 4.

In some embodiments, L^{RN} is —CH₂—, —CO—, or —SO₂—. In some embodiments, L^{RN} is —CH₂—. In some embodiments, L^{RN} is —CO—. In some embodiments, L^{RN} is —SO₂—. In some embodiments, L^{RN} is optionally substituted bivalent C₁₋₄ alkylene. In some embodiments, L^{RN} is optionally substituted bivalent linear C₁₋₄ alkylene. In some embodiments, L^{RN} is —CH₂—CH₂—. In some embodiments, L^{RN} is —CH₂—CH₂—CH₂—. In some embodiments, L^{RN} is —C(CH₃)—.

In some embodiments, R^{RN} is R as described herein. In some embodiments, R^{RN} is C₁₇ alkyl or heteroalkyl having 1-4 heteroatoms, wherein the alkyl or heteroalkyl is optionally substituted with one or more groups independently selected from halogen, a C₅₋₆ aromatic ring having 0-4 heteroatoms, and an optionally substituted 3-10 membered cycloalkyl or heteroalkyl ring having 1-4 heteroatoms.

In some embodiments, R (e.g., R^{RN}, R′, etc.) is optionally substituted aliphatic, e.g., C₁-10 aliphatic. In some embodiments, R is optionally substituted alkyl, e.g., C₁₋₁₀ alkyl. In some embodiments, R is optionally substituted cycloalkyl, e.g., C₁₋₁₀ cycloalkyl. In some embodiments, R is optionally substituted aryl. In some embodiments, R is optionally substituted heterocyclyl. In some embodiments, R is optionally substituted heteroaryl. In some embodiments, is methyl. In some embodiments, R is —CF₃. In some embodiments, R is ethyl. In some embodiments, R is

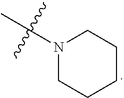 [SMILES: *N1CCCCC1]

In some embodiments, R is phenyl. In some embodiments, R is pentafluorophenyl. In some embodiments, R is pyridinyl.

In some embodiments, one or more R^{a5} are independently —H. In some embodiments, one or more R^{a5} are independently optionally substituted C₁₋₆ alkyl. In some embodiments, each R^{a5} is —H.

In some embodiments, -L^{RN}-R^{RN} is R, and is taken together with a R^{a5} and their intervening atoms to form an optionally substituted, 3-30 membered, monocyclic, bicyclic or polycyclic ring having, in addition to the intervening atoms, 0-10 heteroatoms.

As described in the present disclosure, various rings, including those in various moieties (e.g., R or various groups that can be R, various bivalent rings such as those in -Cy-) and those formed by two entities (e.g., two groups that are or can be R) taken together with their intervening forms, can be various sizes, e.g., 3-30. In some embodiments, a ring is 3-30-membered. In some embodiments, a ring is 3-20 membered. In some embodiments, a ring is 3-10 membered. In some embodiments, a ring is e.g., 3, 4, 5, 6, 7, 8, 9, or 10-membered. In some embodiments, a ring is 3-membered. In some embodiments, a ring is 4-membered. In some embodiments, a ring is 5-membered. In some embodiments, a ring is 6-membered. In some embodiments, a ring is 7-membered. In some embodiments, a ring is 8-membered. In some embodiments, a ring is 9-membered. In some embodiments, a ring is 10-membered. In some embodiments, a ring is substituted (in addition to potential groups already drawn out in formulae). In some embodiments, a ring is not substituted. In some embodiments, a ring is saturated. In some embodiments, a ring is partially unsaturated. In some embodiments, a ring is aromatic. In some embodiments, a ring comprise one or more, e.g., 1-5, heteroatoms. In some embodiments, one or more heteroatoms are oxygen. In some embodiments, one or more heteroatoms are nitrogen. In some embodiments, one or more heteroatoms are sulfur. In some embodiments, a ring is a cycloaliphatic, e.g., cycloalkyl ring. In some embodiments, a ring is a heterocycloaliphatic, e.g., heterocycloalkyl ring. In some embodiments, a ring is an aryl ring. In some embodiments, a ring is a heteroaryl ring. In some embodiments, a ring is a heteroaryl ring. In some embodiments, a ring is monocyclic. In some embodiments, a ring is bicyclic or polycyclic. In some embodiments, each monocyclic unit in a ring is independently an optionally substituted, 3-10 membered (e.g., 3, 4, 5, 6, 7, 8, 9, or 10-membered), saturated, partially unsaturated or aromatic ring having 0-5 heteroatoms.

As described herein, in some embodiments, a heteroatom is selected from nitrogen, oxygen, sulfur, silicon and phosphorus. As described herein, in some embodiments, a heteroatom is selected from nitrogen, oxygen, and sulfur.

In some embodiments, R^{a1} is —H. In some embodiments, R^{a1} is optionally substituted C₁₋₆ alkyl. In some embodiments, R^{a1} are taken together with another group, e.g., R^{a3} and their intervening atoms to form an optionally substituted ring as described herein.

In some embodiments, —C(O)R^{PC} is a protected carboxylic acid group. In some embodiments, —C(O)R^{PC} is an activated carboxylic acid group. Those skilled in the art will appreciate that various groups are available for protecting/activating carboxyl groups, including various groups that are useful in peptide synthesis, and can be utilized in accordance with the present disclosure. In some embodiments, —C(O)R^{PC} is an ester. In some embodiments, —C(O)R^{PC} is an activated ester for synthesis. In some embodiments, —C(O)R^{C} is —C(O)OR′. In some embodiments, R′ is R. In some embodiments, R′ is optionally substituted C₁₋₁₀ aliphatic. In some embodiments, R′ optionally substituted phenyl. In some embodiments, R′ is pentafluorophenyl. In some embodiments, R′ is

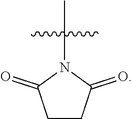 [SMILES: *N1C(=O)CCC1=O]

In some embodiments, —C(O)R^{PC} is —COOH.

In some embodiments, —C(O)R^{PS} is a protected carboxylic acid group. In some embodiments, —C(O)R^{PS} is an activated carboxylic acid group if it is to be reacted with another moiety. Those skilled in the art will appreciate that various groups are available for protecting/activating carboxyl groups, including various groups that are useful in peptide synthesis, and can be utilized in accordance with the present disclosure. In some embodiments, —C(O)R^{PS} is an ester. In some embodiments, —C(O)R^{PS} is an ester. In some embodiments, —C(O)R^{PS} is —C(O)OR′. In some embodiments, R′ is R. In some embodiments, R is optionally substituted C₁₋₁₀ aliphatic. In some embodiments, R optionally substituted phenyl. In some embodiments, R is optionally substituted t-Bu. In some embodiments, R is t-Bu. In some embodiments, R is benzyl. In some embodiments, R is allyl. In some embodiments, —C(O)R^{PC} is a protected carboxylic acid group that is compatible with peptide synthesis (e.g., Fmoc-based peptide synthesis). In some embodiments, —C(O)R^{PC} is a protected carboxylic acid group which is orthogonal to —C(O)R^{PC} and R^{PA}, and remains intact when —C(O)R^{PC} and/or N(R^{PA})(R^{a1}) are protected, deprotected, and/or reacted (e.g., in peptide synthesis such as Fmoc-based peptide synthesis). In some embodiments, —C(O)R^{PC} is deprotected at a late stage during synthesis, e.g., after a peptide backbone is or is largely constructed such that an unprotected side chain —COOH does not impact synthesis.

In some embodiments, —C(O)R^{PC} is —COOH.

As described above, R^{PA} is —H or an amino protecting group. In some embodiments, R^{PA} is —H. In some embodiments, R^{PA} is an amino protecting group. In some embodiments, R^{PA} is an amino protecting group suitable for peptide synthesis. In some embodiments, R^{PA} is —C(O)—O—R, wherein R is optionally substituted

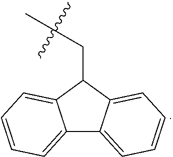 [SMILES: *CC1c2ccccc2-c2ccccc21]

In some embodiments, R^{PA} is -Fmoc. In some embodiments, R^{PA} is —Cbz. In some embodiments, R^{PA} is —Boc.

In some embodiments, R^{PS} is a protecting group orthogonal to R^{PA}. In some embodiments, R^{PS} is a protecting group orthogonal to R^{PC}. In some embodiments, R^{PS} is compatible with peptide synthesis. In some embodiments, R^{PS} is optionally substituted C₁₋₆ aliphatic. In some embodiments, R^{PS} is t-butyl.

In some embodiments, R^{PS} is —S-L-R′, wherein each variable is independently as described herein. In some embodiments, L is optionally substituted —CH₂—. In some embodiments, L is —CH₂—. In some embodiments, R^{PS} is —S—CH₂—R′, wherein R′ is as described herein. In some embodiments, R′ is R as described herein. In some embodiments, R is optionally substituted C₆₋₃₀ aryl. In some embodiments, R is optionally substituted C₆₋₁₀ aryl. In some embodiments, R is optionally substituted phenyl. In some embodiments, R is phenyl. In some embodiments, R is substituted phenyl wherein one or more substituents are independently alkoxy. In some embodiments, R is 2, 4, 6-trimethoxyphenyl. In some embodiments, R is optionally substituted 5-30 membered heteroaryl having 1-10 heteroatoms. In some embodiments, R is optionally substituted 5-10 membered heteroaryl having 1-4 heteroatoms. In some embodiments, R is optionally substituted 5-membered heteroaryl having 1-4 heteroatoms. In some embodiments, R^{PS} is —S—CH₂-Cy-R′, wherein the —CH₂— is optionally substituted, and -Cy- is as described herein. In some embodiments, R^{PS} is —S—CH₂-Cy-O—R′, wherein the —CH₂— is optionally substituted, and -Cy- is as described herein. In some embodiments, -Cy- is an optionally substituted aromatic ring. In some embodiments, -Cy- is optionally substituted phenylene. In some embodiments, -Cy- is 2, 6-dimethoxy-1, 4-phenylene. In some embodiments, -Cy- is 2, 4, 6-trimethoxy-1, 3-phenylene. In some embodiments, R^{PS} is

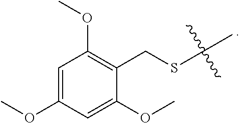 [SMILES: *SCc1c(OC)cc(OC)cc1OC]

In some embodiments, R^{PS} is —SH.

In some embodiments, R^{a2} is

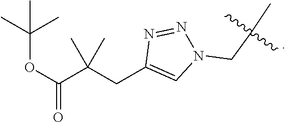 [SMILES: *Cn1cc(CC(C)(C)C(=O)OC(C)(C)C)nn1]

In some embodiments, R^{a2} is

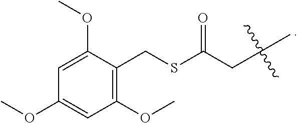 [SMILES: *CC(=O)SCc1c(OC)cc(OC)cc1OC]

In some embodiments, R^{a2} is

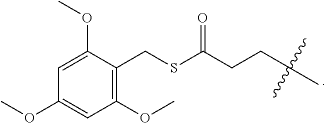 [SMILES: *CCC(=O)SCc1c(OC)cc(OC)cc1OC]

In some embodiments, R^{a2} is

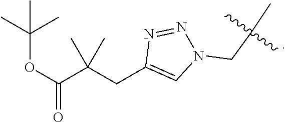 [SMILES: *Cn1cc(CC(C)(C)C(=O)OC(C)(C)C)nn1]

In some embodiments, —C(R^{a2})(R^{a3})— is

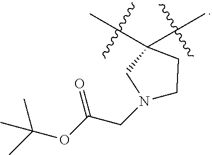

In some embodiments, a provided compound, e.g., an amino acid is selected from:

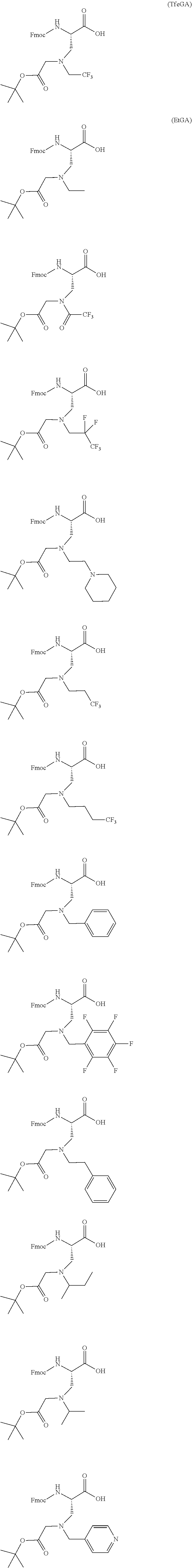

In some embodiments, R^{a2} is R^{a2} in a compound described above (a non-hydrogen group attached to an alpha carbon).

In some embodiments, the present disclosure provides compounds having the structure of:

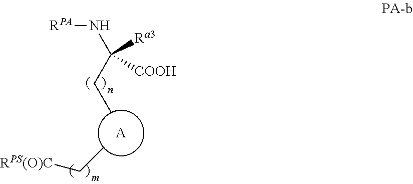

or a salt thereof, wherein:
Ring A is an optionally substituted 3-10 membered ring;
n is 0−6;
m is 0−6.

In some embodiments, m is 0. In some embodiments, m is 1−6.

In some embodiments, the present disclosure provides compounds having the structure of:

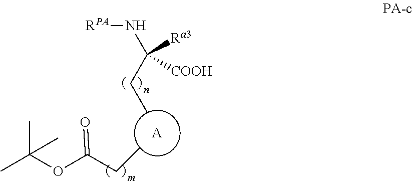

or a salt thereof, wherein:
Ring A is an optionally substituted 3-10 membered ring;
n is 0−6; and
m is 0−6.

In some embodiments, m is 0. In some embodiments, m is 1−6.

In some embodiments, the present disclosure provides compounds having the structure of:

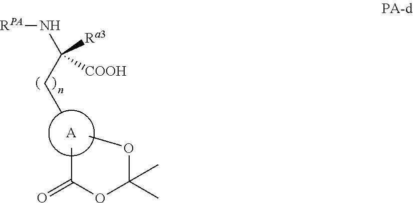

or a salt thereof, wherein:
Ring A is an optionally substituted 3-10 membered ring; and
n is 0−6.

In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 0, 1, or 2.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 1, 2, or 3.

In some embodiments, Ring A is a ring as described herein. In some embodiments, Ring A is 3-membered. In some embodiments, Ring A is 4-membered. In some embodiments, Ring A is 5-membered. In some embodiments, Ring A is 6-membered. In some embodiments, Ring A is 7-membered. In some embodiments, Ring A is 8-membered. In some embodiments, Ring A is 9-membered. In some embodiments, Ring A is 10-membered. In some embodiments, Ring A is saturated. In some embodiments, Ring A is partially unsaturated. In some embodiments, Ring A is aromatic. In some embodiments, Ring A has no additional heteroatoms in addition to the nitrogen atom. In some embodiments, Ring is unsubstituted. In some embodiments, Ring A is substituted with one or more halogen. In some embodiments, Ring A is substituted with one or more —F. In some embodiments, Ring A has a carbon substituted with two —F. In some embodiments, —C(O)R^{PS} is at 2′-position (N being position 1). In some embodiments, —C(O)R^{PS} is at 3′-position. In some embodiments, —C(O)R^{PS} is at 4′-position. In some embodiments, —C(O)R^{PS} is attached to a chiral center, e.g., a chiral carbon atom. In some embodiments, a chiral center is R. In some embodiments, a chiral center is S. In some embodiments, Ring A is bonded to —(CH₂)ₙ— at a chiral carbon which is R. In some embodiments, Ring A is bonded to —(CH₂)ₙ— at a chiral carbon which is S. In some embodiments, —(CH₂)ₙ— is at position 2 (the N is at position 1). In some embodiments, —(CH₂)ₙ— is at position 3 (the N is at position 1). In some embodiments, —(CH₂)ₙ— is at position 4 (the N is at position 1).

In some embodiments, Ring A is substituted. In some embodiments, substituents on Ring A are of suitable properties, e.g., volumes, for various utilizations. In some embodiments, substituents are independently selected from halogen, —R, —CF₃, —N(R)₂, —CN, and —OR, wherein each R is independently C₁₋₆ aliphatic optionally substituted with one or more —F. In some embodiments, substituents are independently selected from halogen, C₁₋₅ linear, branched or cyclic alkyl, —OR wherein R is C₁₋₄ linear, branched or cyclic alkyl, fluorinated alkyl, —N(R)₂ wherein each R is independently C₁₋₆ linear, branched or cyclic alkyl, or —CN. In some embodiments, substituents are selected from halogen, a C₅₋₆ aromatic ring having 0-4 heteroatoms, and an optionally substituted 3-10 membered cycloalkyl or heteroalkyl ring having 1-4 heteroatoms. In some embodiments, a substituent is halogen. In some embodiments, it is —F. In some embodiments, it is —Cl. In some embodiments, it is -Br. In some embodiments, it is —I. In some embodiments, a substituent is optionally substituted C₁₋₄ alkyl. In some embodiments, a substituent is C₁₋₄ alkyl. In some embodiments, it is methyl. In some embodiments, it is ethyl. In some embodiments, it is i-Pr. In some embodiments, a substituent is C₁₋₄ haloalkyl. In some embodiments, a substituent is C₁₋₄ alkyl optionally substituted with one or more —F. In some embodiments, it is —CF₃. In some embodiments, it is —CN. In some embodiments, it is —OR wherein R is optionally substituted C₁₋₄ alkyl. In some embodiments, it is —OR wherein R is C₁₋₄ alkyl. In some embodiments, it is —OR wherein R is C₁₋₄ haloalkyl. In some embodiments, it is —OR wherein R is C₁₋₄ alkyl optionally substituted with one or more —F. In some embodiments, it is —OCF₃.

In some embodiments, Ring A is or comprises an optionally substituted saturated monocyclic ring. In some embodiments, Ring A is or comprises an optionally substituted partially unsaturated monocyclic ring. In some embodiments, Ring A is or comprises an optionally substituted aromatic monocyclic ring. In some embodiments, Ring A is optionally substituted phenyl. In some embodiments, Ring A is optionally substituted 5−6 membered heteroaryl having 1-3 heteroatoms. In some embodiments, Ring A is optionally substituted 5−6 membered heteroaryl having 1-3 heteroatoms, wherein at least one heteroatom is nitrogen. In some embodiments, Ring A is an optionally substituted 8-10 membered bicyclic ring having 1−6 heteroatoms. In some embodiments, Ring A is an optionally substituted 8-10 membered bicyclic aromatic ring having 1−6 heteroatoms, wherein each monocyclic unit is independently an optionally 5−6 membered aromatic ring having 0-3 heteroatoms. In some embodiments, Ring A is bonded to —(CH₂)ₙ— at a carbon atom. In some embodiments, Ring A is bonded to —(CH₂)ₙ— at a nitrogen atom. In some embodiments, Ring A or -Cy- in L^{aa} is optionally substituted, and each substitute is independently selected from halogen, —R, —CF₃, —N(R)₂, —CN, and —OR, wherein each R is independently C₁₋₆ aliphatic optionally substituted with one or more —F. In some embodiments, Ring A or -Cy- in L^{aa} is optionally substituted, and each substitute is independently selected from halogen, C₁₋₅ linear, branched or cyclic alkyl, —OR wherein R is C₁₋₄ linear, branched or cyclic alkyl, fluorinated alkyl, —N(R)₂ wherein each R is independently C₁₋₆ linear, branched or cyclic alkyl, or —CN.

In some embodiments, Ring A is optionally substituted phenyl. In some embodiments, the present disclosure provides a compound of formula

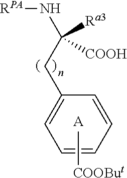

or a salt thereof, wherein Ring A is optionally substituted phenyl, and each variable is as described herein.

In some embodiments, the present disclosure provides compounds having the structure of

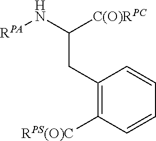

or a salt thereof, wherein each variable is independent as described herein. In some embodiments, the present disclosure provides compounds having the structure of

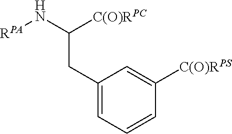

or a salt thereof, wherein each variable is independent as described herein.

In some embodiments, a compound is selected from:

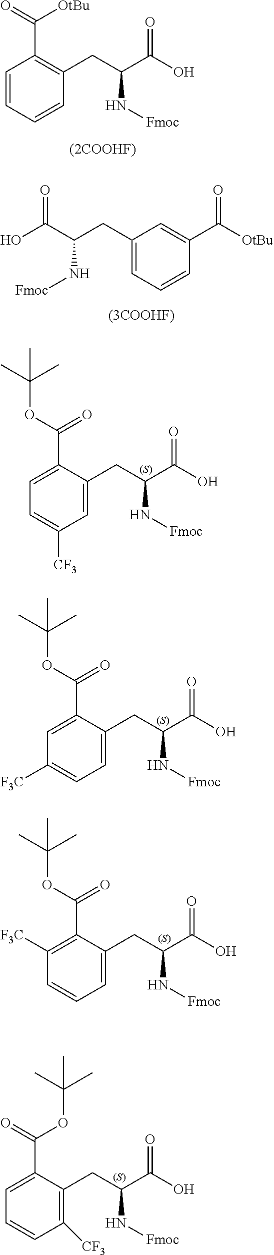

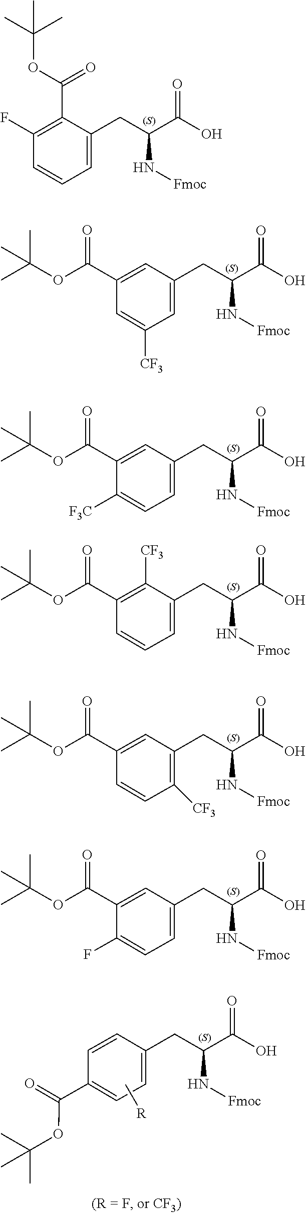

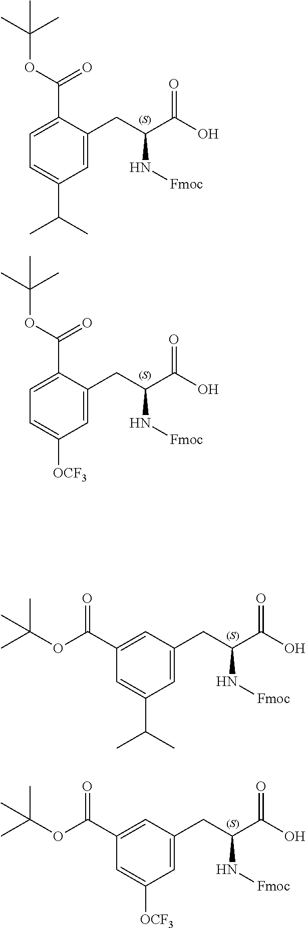

In some embodiments, the present disclosure provides a compound of formula

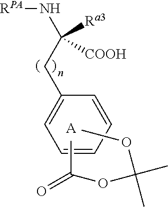

or a salt thereof, wherein Ring A is optionally substituted phenyl, and each variable is as described herein. In some embodiments, a compound is selected from:

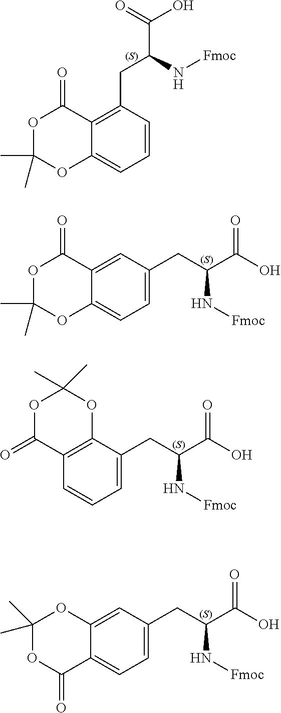

In some embodiments, Ring A is an optionally substituted 5- or 6-membered heteroaryl having 1-4 heteroatoms. In some embodiments, a provided compound has the structure of

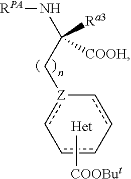

wherein Z is carbon or a heteroatom, Ring Het is an optionally substituted 5- or 6-membered heteroaryl having 1-4 heteroatoms, and each other variable is independently as described herein. In some embodiments, a provided compound is selected from:

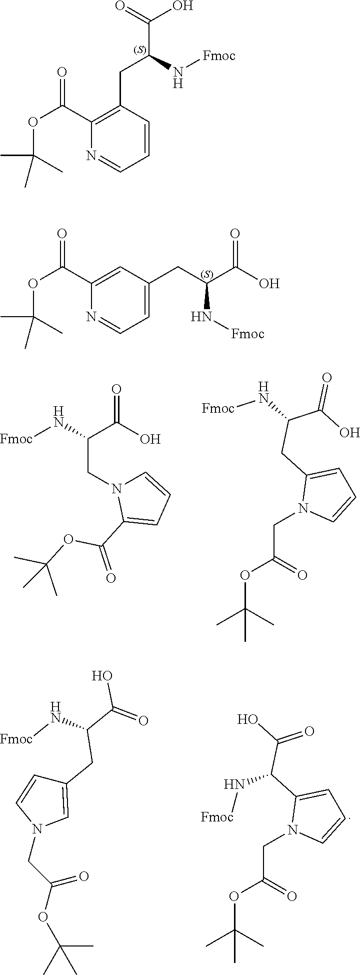

In some embodiments, Ring A is a 8-10 membered bicyclic aryl or a heteroaryl ring having 1-5 heteroatoms. In some embodiments, Ring A is a 10-membered bicyclic aryl ring. In some embodiments, Ring A is a 8-membered bicyclic heteroaryl ring having 1-5 heteroatoms. In some embodiments, Ring A is a 9-membered bicyclic heteroaryl ring having 1-5 heteroatoms. In some embodiments, Ring A is a 10-membered bicyclic heteroaryl ring having 1-5 heteroatoms. In some embodiments, Ring A is an optionally substituted 5- or 6-membered heteroaryl having 1-4 heteroatoms.

In some embodiments, a provided compound has the structure of

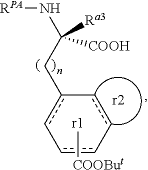

wherein each of Ring r1 and r2 is independently an optionally substituted 5- or 6-membered aryl or heteroaryl ring having 1-4 heteroatoms, and each other variable is independently as described herein. In some embodiments, a provided compound has the structure of

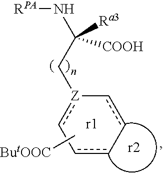

wherein Z is carbon or a heteroatom, each of Ring r1 and r2 is independently an optionally substituted 5- or 6-membered aryl or heteroaryl ring having 1-4 heteroatoms, and each other variable is independently as described herein. In some embodiments, a provided compound is selected from:

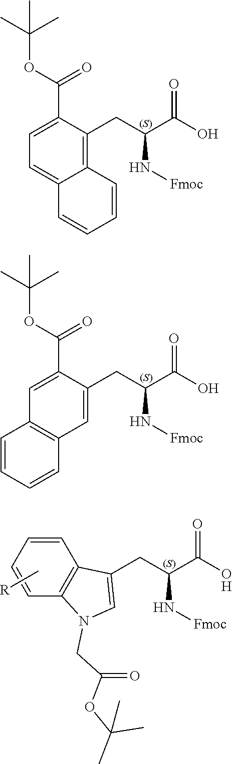

In some embodiments, the present disclosure provides a compound of structure

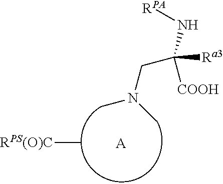

or a salt thereof. In some embodiments, —C(O)R^{PS} is —C(O)—OtBu. In some embodiments, the present disclosure provides a compound of structure

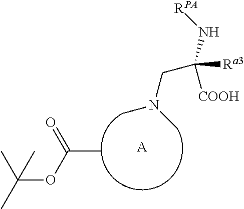

or a salt thereof, wherein each variable is independently as described herein.

In some embodiments, a provided compound is selected from:

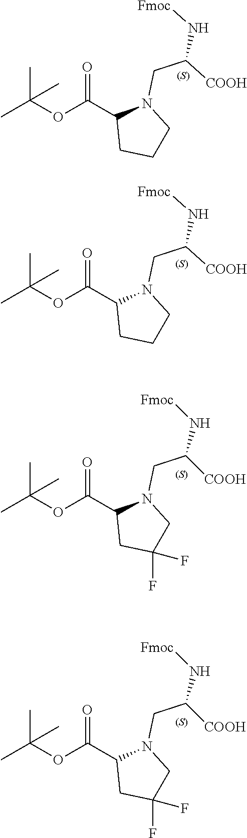

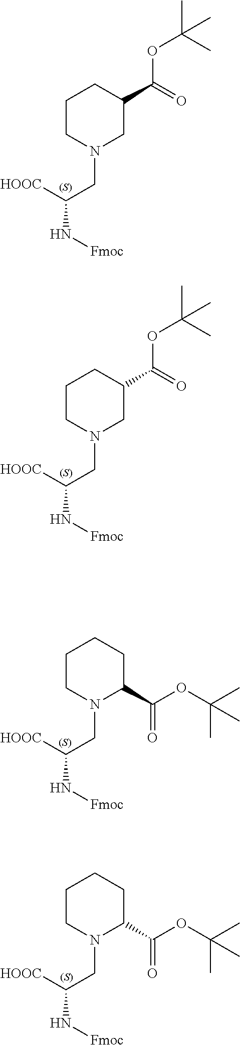

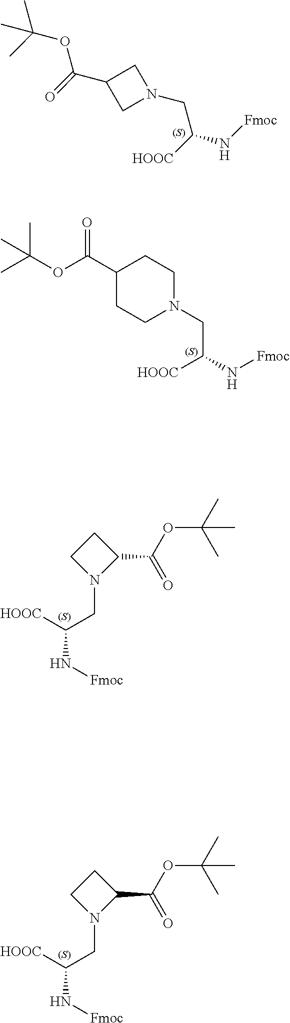

In some embodiments, the present disclosure provides compounds having the structure of

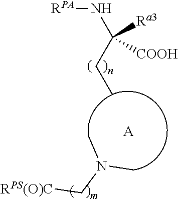

or a salt thereof, wherein each variable is independently as described herein. In some embodiments, the present disclosure provides compounds having the structure of

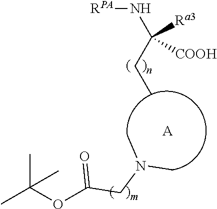

or a salt thereof, wherein each variable is independently as described herein.

In some embodiments, a provided compound is selected from:

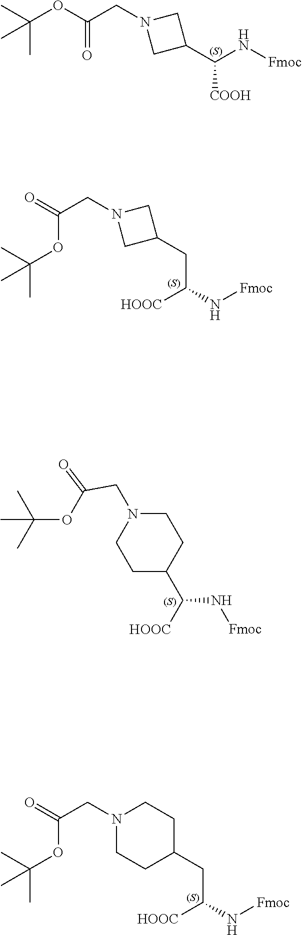

In some embodiments, a provided compound is an amino acid. In some embodiments, a provided compound is a protected amino acid. In some embodiments, a provided compound is a protected and/or activated amino acid. In some embodiments, a provided compound is suitable for

In some embodiments, a ring moiety of, e.g., -Cy-, R (including those formed by R groups taken together), etc. is monocyclic. In some embodiments, a ring moiety is bicyclic or polycyclic. In some embodiments, a monocyclic ring is an optionally substituted 3-10 (3, 4, 5, 6, 7, 8, 9, or 10, 3-8, 3-7, 4-7, 4−6, 5−6, etc.) membered, saturated, partially unsaturated or aromatic ring having 0-5 heteroatoms. In some embodiments, each monocyclic ring unit of a bicyclic or polycyclic ring moiety is independently an optionally substituted 3-10 (3, 4, 5, 6, 7, 8, 9, or 10, 3-8, 3-7, 4-7, 4−6, 5−6, etc.) membered, saturated, partially unsaturated or aromatic ring having 0-5 heteroatoms.

In some embodiments, each heteroatom is independently selected from oxygen, nitrogen, sulfur, phosphorus and silicon. In some embodiments, each heteroatom is independently selected from oxygen, nitrogen, and sulfur.

In some embodiments, L^{a1} is a covalent bond. In some embodiments, a compound of formula PA is of the structure NH(R^{a1})—C(R^{a2})(R^{a3})-L^{a2}-COOH.

In some embodiments, L^{a2} is a covalent bond. In some embodiments, a compound of formula PA is of the structure NH(R^{a1})—C(R^{a2})(R^{a3})-L^{a2}-COOH.

In some embodiments, L^{a1} is a covalent bond and L^{a2} is a covalent bond. In some embodiments, a compound of formula PA is of the structure NH(R^{a1})—C(R^{a2})(R^{a3})—COOH.

In some embodiments, an amino acid is suitable for stapling. In some embodiments, an amino acid comprises a terminal olefin.

In some embodiments, an amino acid has the structure of NH(R^{a1})-L^{a1}-C(-L^{aa}-COOH)(R^{a3})-L^{a2}-COOH, or a salt thereof, wherein each variable is independently as described in the present disclosure. In some embodiments, L^{aa} is -L^{am1}-N(R′)-L^{am2}-, wherein each variable is as described herein. In some embodiments, each of L^{am1} and L^{am2} is optionally substituted bivalent C₁₋₆ aliphatic. In some embodiments, each of L^{am1} and L^{am2} is bivalent C₁₋₆ aliphatic. In some embodiments, each of L^{am1} and L^{am2} is optionally substituted bivalent C₁₋₆ alkyl. In some embodiments, each of L^{am1} and L^{am2} is bivalent C₁₋₆ alkyl. In some embodiments, each of L^{am1} and L^{am2} is optionally substituted bivalent linear C₁₋₆ alkyl. In some embodiments, each of L^{am1} and L^{am2} is bivalent linear C₁₋₆ alkyl. In some embodiments, L^{am1} is —CH₂—. In some embodiments, L^{am2} is a covalent bond. In some embodiments, L^{am2} is —CH₂—. In some embodiments, both L^{am1}and L^{am2} are —CH₂—. In some embodiments, L^{am1} is —CH₂— and L^{am2} is a covalent bond. In some embodiments, —N(R′)— is —N(Et)-. In some embodiments, —N(R′)— is —N(CH₂CF₃)—. In some embodiments, L^{aa} is -L^{am1}-Cy-L^{am2}-, wherein each variable is as described herein. In some embodiments, -Cy- is optionally substituted phenyl. In some embodiments, -Cy- is optionally substituted 5−6 membered heteroaryl having 1-4 heteroatoms.

In some embodiments, a compound is

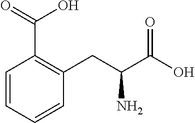 [SMILES: N[C@@H](Cc1ccccc1C(=O)O)C(=O)O]

(2COOHF) or a salt thereof. In some embodiments, a compound is

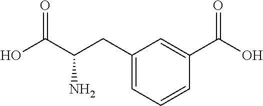 [SMILES: N[C@@H](Cc1cccc(C(=O)O)c1)C(=O)O]

(3COOHF) or a salt thereof. In some embodiments, a compound is

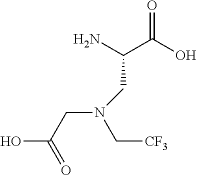 [SMILES: N[C@@H](CN(CC(=O)O)CC(F)(F)F)C(=O)O]

(TfeGA) or a salt thereof. In some embodiments, a compound is

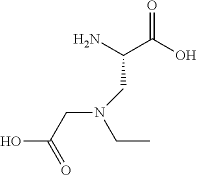

(EtGA) or a salt thereof. In some embodiments, a compound is

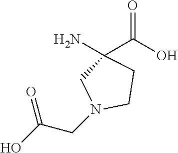 [SMILES: N[C@@]1(C(=O)O)CCN(CC(=O)O)C1]

or a salt thereof. In some embodiments, a compound is

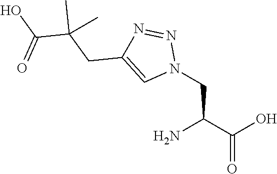 [SMILES: CC(C)(Cc1cn(C[C@H](N)C(=O)O)nn1)C(=O)O]

or a salt thereof. In some embodiments, a compound is

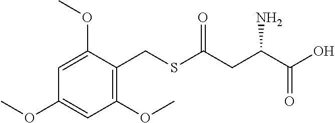 [SMILES: COc1cc(OC)c(CSC(=O)C[C@H](N)C(=O)O)c(OC)c1]

or a salt thereof. In some embodiments, a compound is

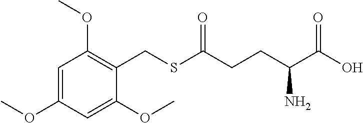 [SMILES: COc1cc(OC)c(CSC(=O)CC[C@H](N)C(=O)O)c(OC)c1]

or a salt thereof. In some embodiments, a compound is

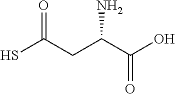

or a salt thereof. In some embodiments, a compound is

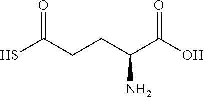

or a salt thereof. Among other things, such compounds may be utilized as amino acid residues in peptides including stapled peptides.

In some embodiments, the present disclosure provides a compound, e.g., a peptide, comprising a residue of a compound of formula PA or a salt form thereof. In some embodiments, a residue has the structure of —N(R^{a1})-L^{a1}-C(R^{a2})(R^{a3})-L^{a2}-C(O)— or a salt form thereof, wherein each variable is independently as described herein. In some embodiments, a residue has the structure of —N(R^{a1})-L^{a1}-C(-L^{aa}-COOH)(R^{a1})-L^{a2}-C(O)— or a salt form thereof, wherein each variable is independently as described herein. For example, in some embodiments, a residue is

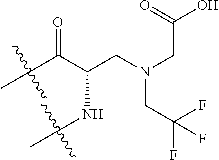 [SMILES: *N[C@@H](CN(CC(=O)O)CC(F)(F)F)C(*)=O]

or a salt form thereof. In some embodiments, a residue is

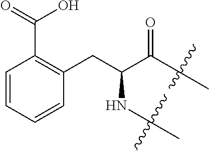 [SMILES: *N[C@@H](Cc1ccccc1C(=O)O)C(*)=O]

or a salt form thereof. In some embodiments, a residue is

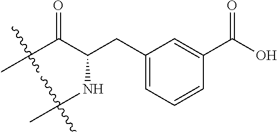 [SMILES: *N[C@@H](Cc1cccc(C(=O)O)c1)C(*)=O]

or a salt form thereof. In some embodiments, a residue is

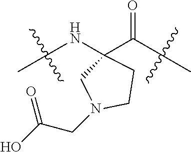 [SMILES: *N[C@@]1(C(*)=O)CCN(CC(=O)O)C1]

or a salt form thereof. In some embodiments, a residue is

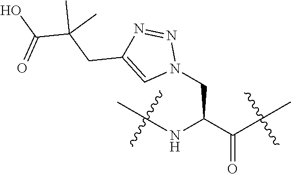 [SMILES: *N[C@@H](Cn1cc(CC(C)(C)C(=O)O)nn1)C(*)=O]

or a salt form thereof. In some embodiments, a residue is

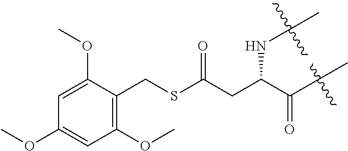 [SMILES: *N[C@@H](CC(=O)SCc1c(OC)cc(OC)cc1OC)C(*)=O]

or a salt form thereof. In some embodiments, a residue is

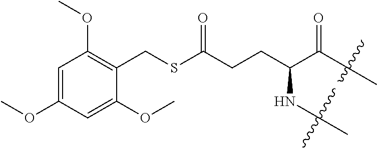 [SMILES: *N[C@@H](CCC(=O)SCc1c(OC)cc(OC)cc1OC)C(*)=O]

or a salt form thereof. In some embodiments, a residue is

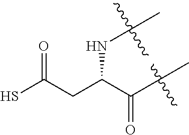 [SMILES: *N[C@@H](CC(=O)S)C(*)=O]

or a salt form thereof. In some embodiments, a residue is

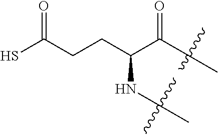 [SMILES: *N[C@@H](CCC(=O)S)C(*)=O]

or a salt form thereof.

In some embodiments, an amino acid, or a structure moiety of an amino acid or an agent (e.g., a peptide), is selected from below. Brackets indicate that a moiety is attached at R₃ position of an amino acid, for instance, for a Lys with a bracket moiety

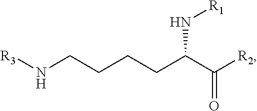

a bracket moiety would replace R₃ (when there is no bracket preceding an amino acid, e.g., Lys, then R₃ is hydrogen). For a bracket structure, R or R₁ indicate a moiety connected to the bracket structure (for

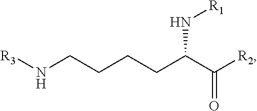

R would be

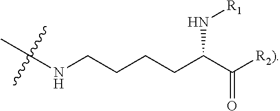

For example, a [1NapPr]Lys residue has the structure of

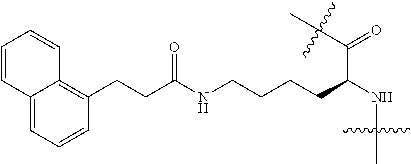 [SMILES: *N[C@@H](CCCCNC(=O)CCc1cccc2ccccc12)C(*)=O]

### ([1NaPr] is

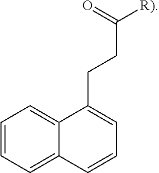

R₁ is the N-term (and connects to the carboxylate of the previous amino acid residue, or a N-terminal cap, or is —H for an amino acid). R₂ is the C-term (and connects to the N-term of the next amino acid residue, or a C-terminal group, or is —OH for an amino acid). R₃ indicates a potential bracket moiety (or is H if no moiety is indicated). Typically, after linear peptide synthesis, the terminal olefins of all residues are linked by ring-closing metathesis.

TABLE A-IV
Certain useful compounds or moieties.

| Compound/ | |
|---|---|
| Bracket Moiety | Structure |
| [1Napc] | 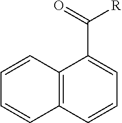 |
| [1NapPr] | 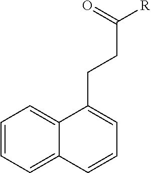 |
| [2Napc] | 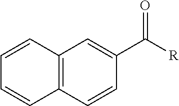 |
| [22PhPr] | 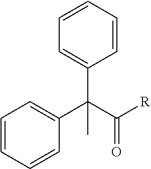 |
| [35CF3PhPr] | 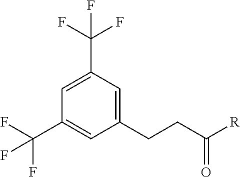 |
| [4MePipBz] | 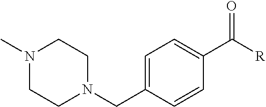 |
| [Acryl] | 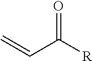 |
| [AdamantC] | 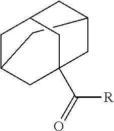 |
| [AdamantPro] | 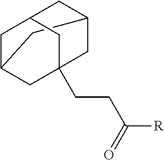 |
| [AzAc] | 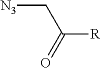 |
| [Bn] | 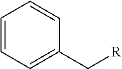 |
| [Bua] | 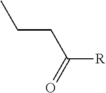 |
| [FAM6Ppg][1TriAc] | 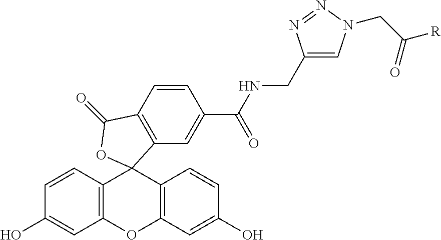 |
| [FAM6Ppg][p1TB] | 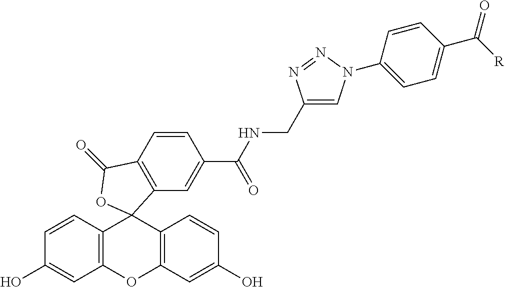 |
| [iPr] | 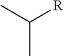 |
| [Me2NCBz] | 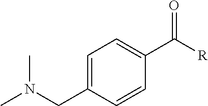 |
| [Me3AdamantC] | 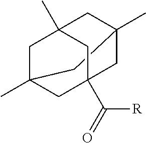 |
| [MeBipipAc] | 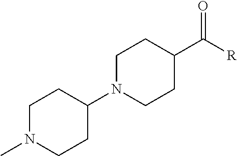 |
| [MeMorphBz] | 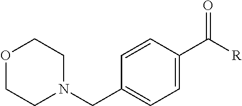 |
| [MePipAc] | 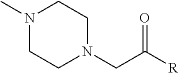 |
| [MeSO2] | 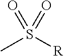 |
| [MorphAc] | 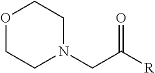 |
| [mPEG2] | 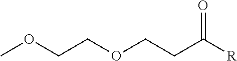 |
| [mPEG4] | 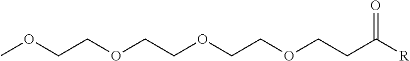 |
| [mPEG6] | 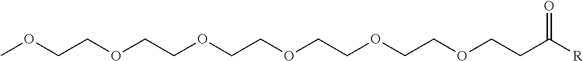 |
| [mPEG8] | 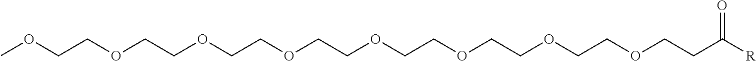 |
| AsnR | 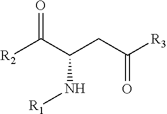 |
| [NHCyBu] | 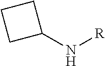 |
| [NHCyHe] | 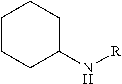 |
| [NHCyPr] | 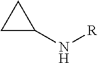 |
| [NHEt] | 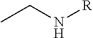 |
| [NHiPr] | 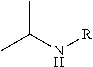 |
| [NHMe] | 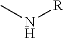 |
| [NHnPr] | 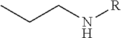 |
| [Oct] | 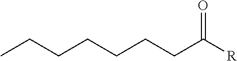 |
| [Pfbn] | 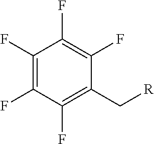 |
| [PropynPEG1] | 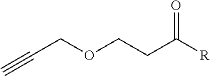 |
| [PropynPEG2] | 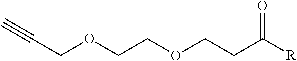 |
| [PropynPEG3] | 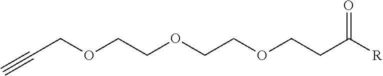 |
| [PropynPEG4] | 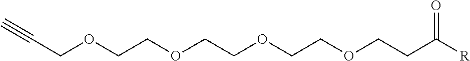 |
| [Tf] | 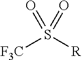 |
| [Tfb] | 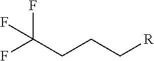 |
| [Tfp] | 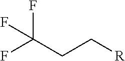 |
| 1AcAW | 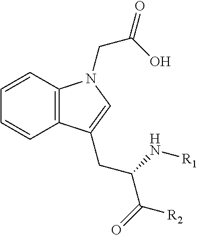 |
| 1meH | 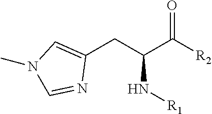 |
| 1MeW | 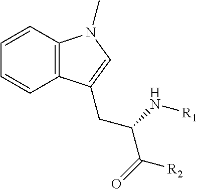 |
| 1NapA | 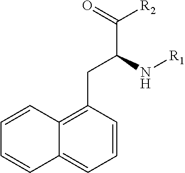 |
| 23FF | 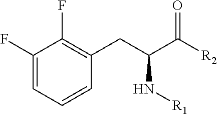 |
| 2BrF | 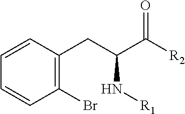 |
| 2CBMF | 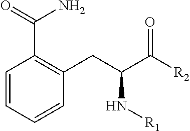 |
| 2ClF | 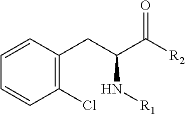 |
| 2CNF | 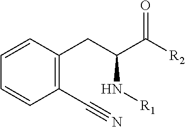 |
| 2COOHF | 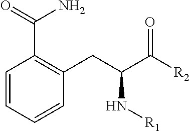 |
| 2F3MeF | 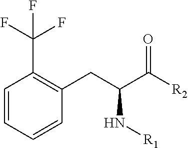 |
| 2FF | 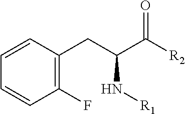 |
| 2FurA | 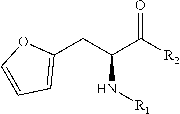 |
| 2MeF | 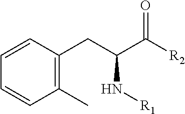 |
| 2NapA | 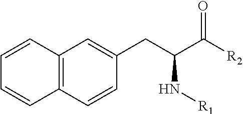 |
| 2NO2F | 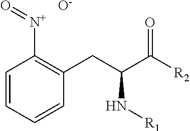 |
| 2OMeF | 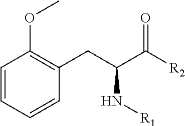 |
| 2pyrA | 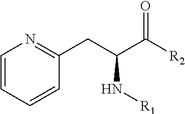 |
| 2Thi | 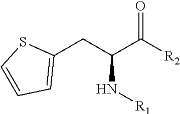 |
| 345FF | 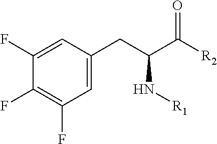 |
| 34FF | 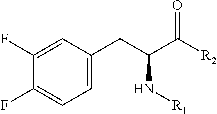 |
| 34OMeF | 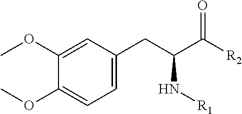 |
| 35FF | 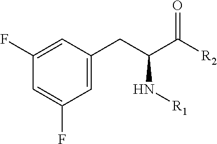 |
| 3CBMF | 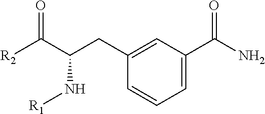 |
| 3ClF | 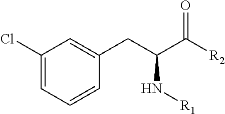 |
| 3CNF | 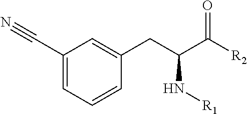 |
| 3COOHF | 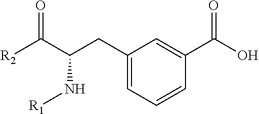 |
| 3F3MeF | 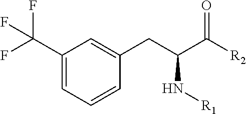 |
| 3FF | 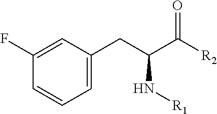 |
| 3MeF | 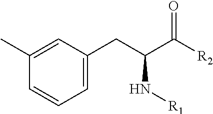 |
| 3meH | 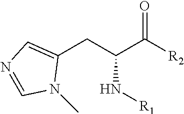 |
| 3OMeF | 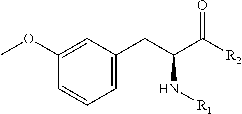 |
| 3pyrA | 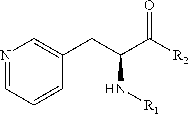 |
| 3Thi | 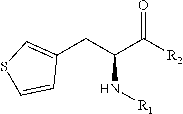 |
| 4AmPhe | 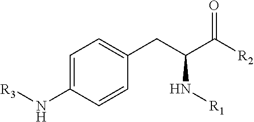 |
| 4CBMF | 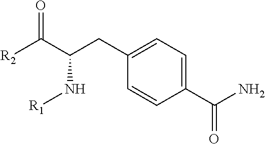 |
| 4ClF | 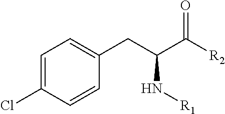 |
| 4CNF | 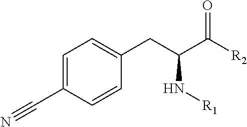 |
| 4COOHF | 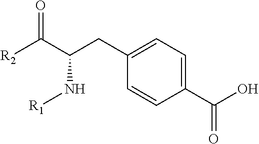 |
| 4F3MeF | 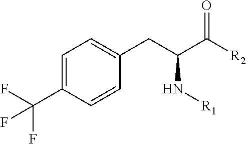 |
| 4FF | 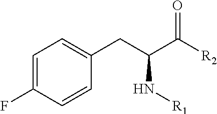 |
| 4MeF | 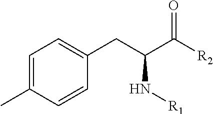 |
| 4pyrA | 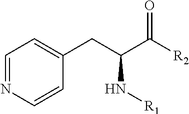 |
| 4Thz | 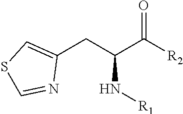 |
| 4TriA | 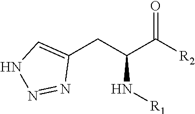 |
| 5CF3W | 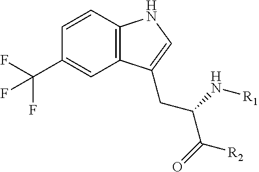 |
| 5ClW | 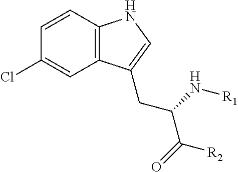 |
| 5CpW | 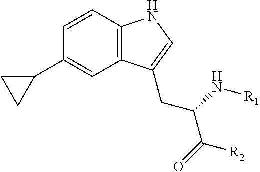 |
| 5FW | 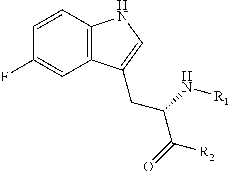 |
| 5MeOW | 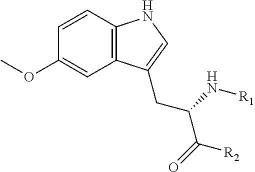 |
| 6AmHex | 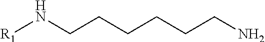 |
| 6AzHex | 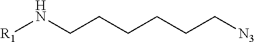 |
| 6ClW | 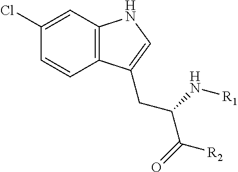 |
| 6F1NapA | 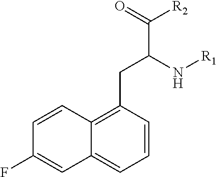 |
| 7F1NapA | 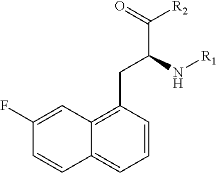 |
| 7FW | 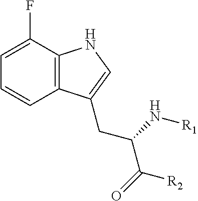 |
| Aad | 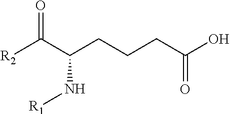 |
| Abu | 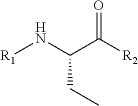 |
| Aib | 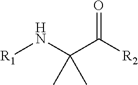 |
| aIle | 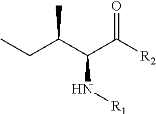 |
| Alaol | 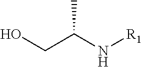 |
| aMeDF | 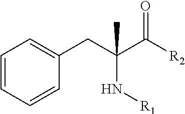 |
| aMeW | 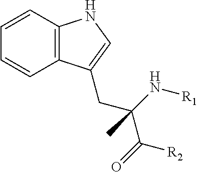 |
| Az | 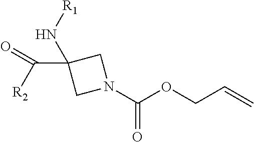 |
| B5 | 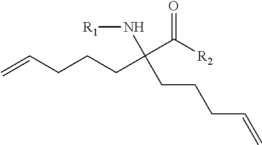 |
| Bip | 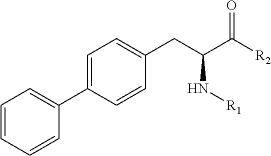 |
| BztA | 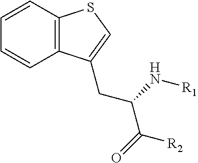 |
| Cha | 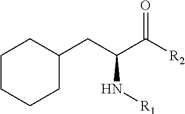 |
| Cit | 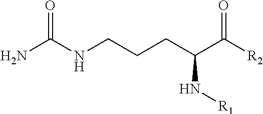 |
| CypA | 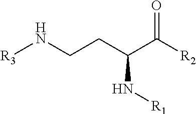 |
| Dab | 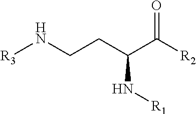 |
| DaMeL | 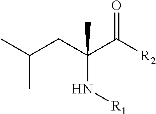 |
| Dap | 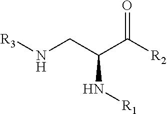 |
| dAsn | 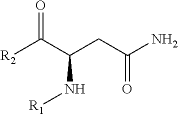 |
| dGln | 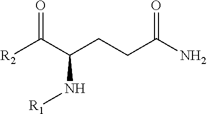 |
| DGlu | 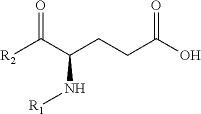 |
| dHis | 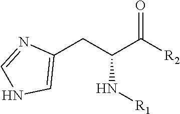 |
| DipA | 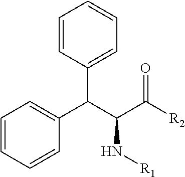 |
| dPhe | 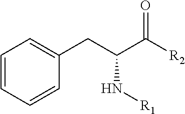 |
| dSer | 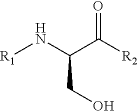 |
| dThr | 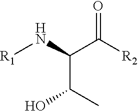 |
| EtGA | 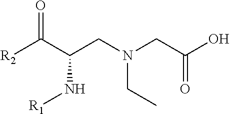 |
| F3CA | 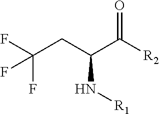 |
| GA | 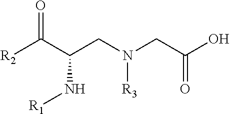 |
| H2Trp | 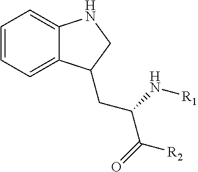 |
| HF2CA | 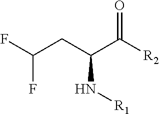 |
| hLeu | 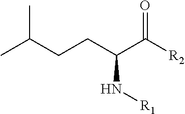 |
| Hleu | same as above |
| hPhe | 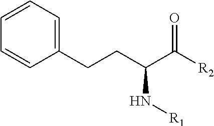 |
| Hse | 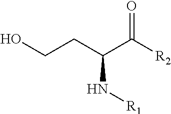 |
| hSe | same as above |
| hTyr | 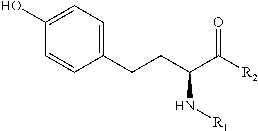 |
| iPrLys | 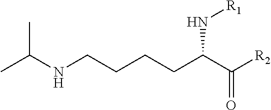 |
| isoAsp | 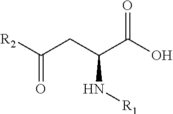 |
| isoDAsp | 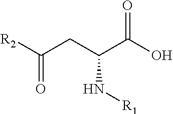 |
| Leuol | 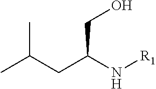 |
| MeOPr | 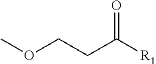 |
| MeTyr | 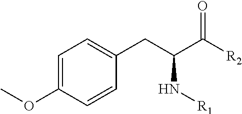 |
| MorphAla | 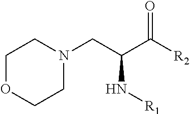 |
| NdiMeButC | 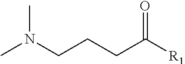 |
| nLeu | 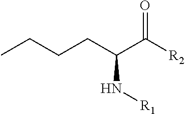 |
| NMeD | 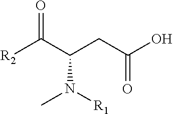 |
| NMeHis | 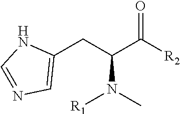 |
| NMeS | 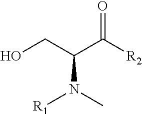 |
| Npg | 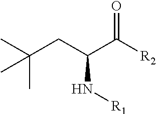 |
| npG | same as above |
| Nva | 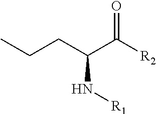 |
| Pff | 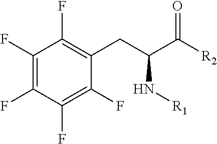 |
| Pheol | 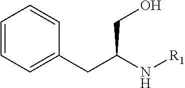 |
| Phg | 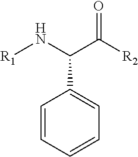 |
| Pic | 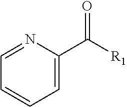 |
| PL3 | 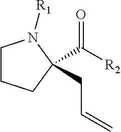 |
| Prool | 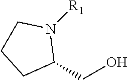 |
| PyrS | 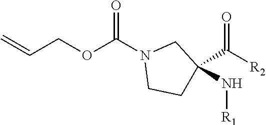 |
| PyrS1 | 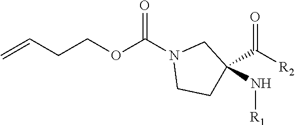 |
| PyrS2 | 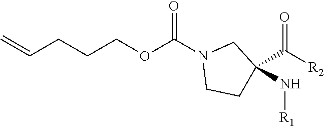 |
| PyrS3 | 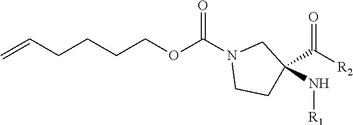 |
| Qui | 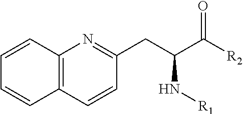 |
| R4 | 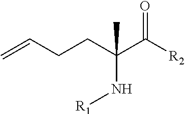 |
| R5 | 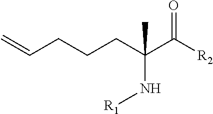 |
| R6 | 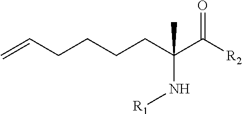 |
| R7 | 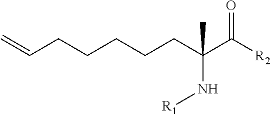 |
| R8 | 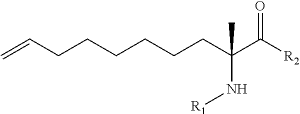 |
| RbGlu | 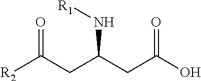 |
| RdN | 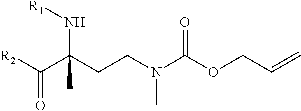 |
| ReN | 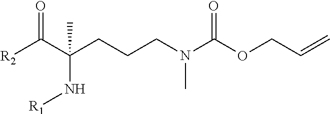 |
| RgN | 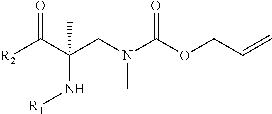 |
| S(Ome) | 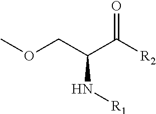 |
| S(Ome) | same as above |
| S10 | 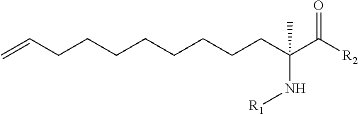 |
| S4 | 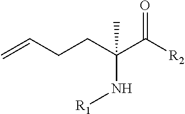 |
| S5 | 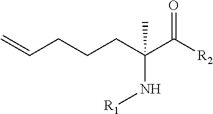 |
| S6 | 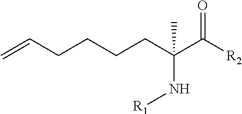 |
| S7 | 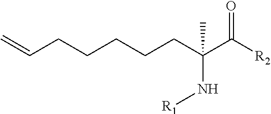 |
| S8 | 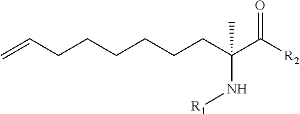 |
| SbGlu | 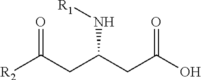 |
| SdN | 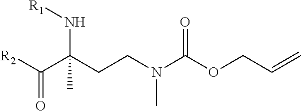 |
| SeN | 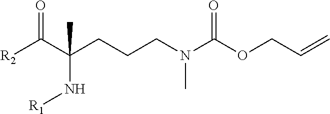 |
| SgN | 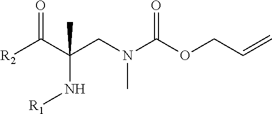 |
| tetz | 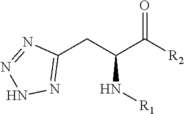 |
| TfeGA | 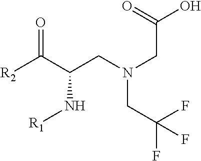 |
| Throl | 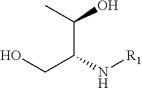 |
| TOMe | 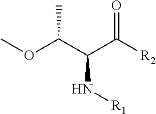 |
| WCHO | 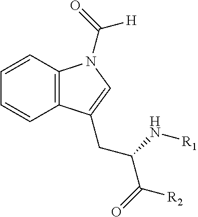 |
| [2COOH4NH2Ph] | 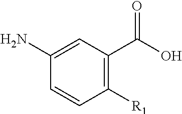 |
| [2COOH4NO2Ph] | 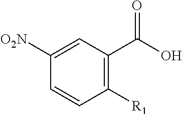 |
| [2COOHPh] | 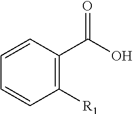 |
| [2Nic] | 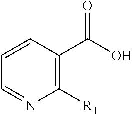 |
| [2OxoPpz] | 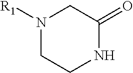 |
| [3C] | 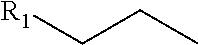 |
| [3Py] | 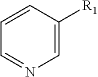 |
| [4AcMePip] | 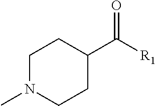 |
| [4CF3PhAc] | 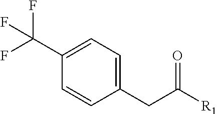 |
| [4F3CPip] | 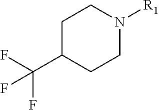 |
| [4MePpzPip] | 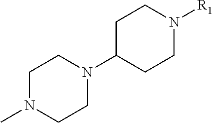 |
| [4Pippip] | 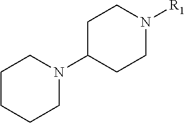 |
| [4PyPip] | 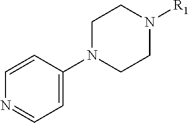 |
| [Ac] | 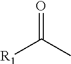 |
| [AcPpz] | 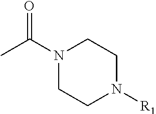 |
| [Allyl] | 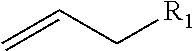 |
| [bismethoxyethylamine] | 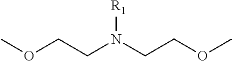 |
| [Bn] | 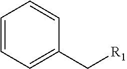 |
| [CCpCO2H] | 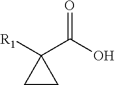 |
| [CF3CO] | 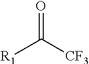 |
| [CH2CChCO2H] | 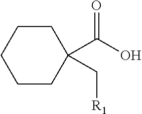 |
| [CH2CCpCO2H] | 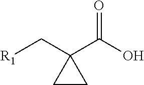 |
| [CH2CH2CO2H] | 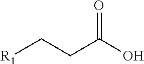 |
| [CH2CMe2CO2H] | 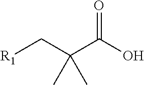 |
| [CH2CO2H] | 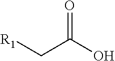 |
| [CH2NMe2] | 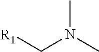 |
| [CH2Ppz] | 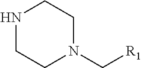 |
| [CMe2CO2H] | 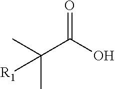 |
| [CyPr] | 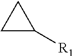 |
| [Et] | 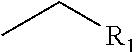 |
| [EtSO2Ppz] | 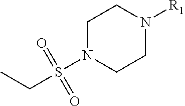 |
| [EtSSEt] | 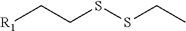 |
| [EtSSHex] | 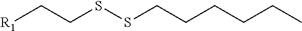 |
| [EtSSPh] | 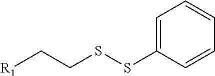 |
| [EtSSpy] | 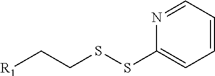 |
| [H4IAP] | 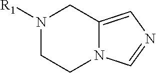 |
| [isoindoline] | 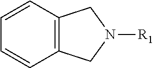 |
| [lithocholate] | 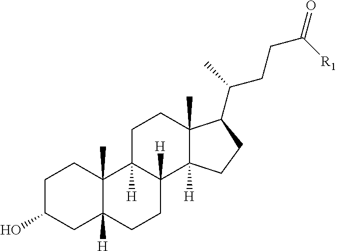 |
| [PEG2] | 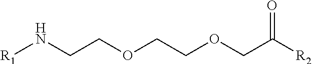 |
| [Me] | R₁— |
| [Me2diaminobutane] | 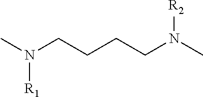 |
| [Me2NCBz] | 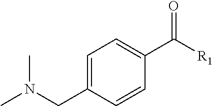 |
| [Me2Npr] | 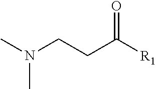 |
| [Me2NPrPip] | 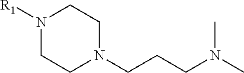 |
| [Me3AdamantC] | 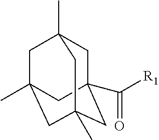 |
| [MeMorphBz] | 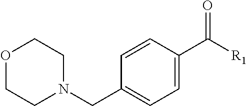 |
| [MePipAc] | 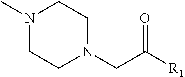 |
| [MeSO2] | 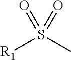 |
| [Morph] | 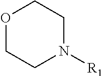 |
| [MorphAc] | 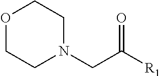 |
| [MorphCH2] | 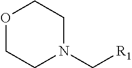 |
| [MorphEt] | 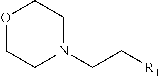 |
| [NdiMeButC] | 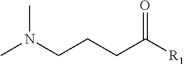 |
| [NHBn] | 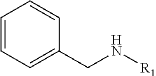 |
| [NHEt] | 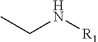 |
| [NMe2] | 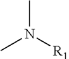 |
| [PfbGA] | 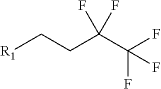 |
| [Pfbn] | 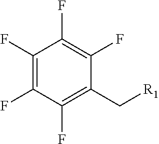 |
| [PfBz] | 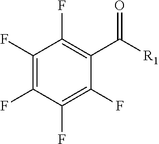 |
| [PfPhAc] | 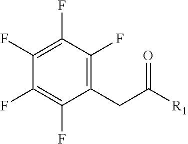 |
| [Ph] | 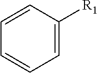 |
| [Phc] | 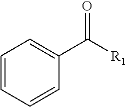 |
| [Pic] | 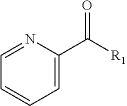 |
| [Ppz] | 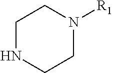 |
| [RDMAPyr] | 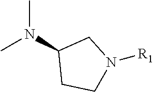 |
| [sBu] | 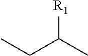 |
| [SO2MorphCH2] | 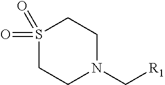 |
| [Tfb] | 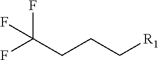 |
| [TfePpz] | 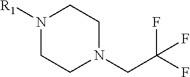 |
| [Tfp] | 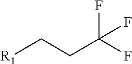 |
| 2F3MeW | 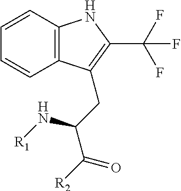 |
| 2NH2F | 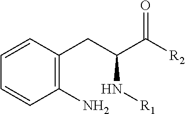 |
| 34ClF | 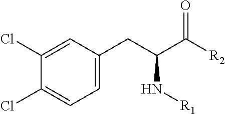 |
| 34MeF | 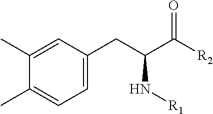 |
| 3Br4FF | 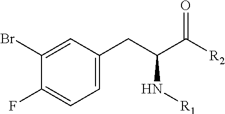 |
| 3BrF | 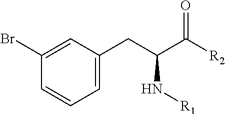 |
| 3butenyl | 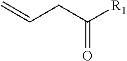 |
| 3CBMF | 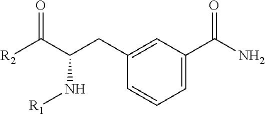 |
| 3CH2NMe2F | 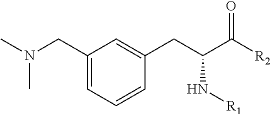 |
| 3CO2PhF | 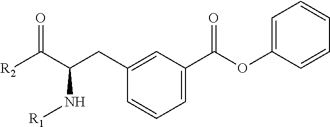 |
| 3SF | 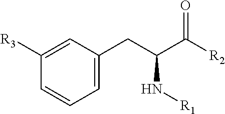 |
| 3SO2F | 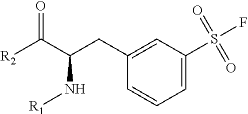 |
| 3TzF | 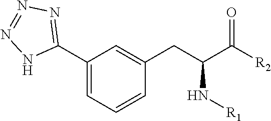 |
| 4BrF | 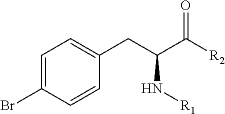 |
| 4ClBztA | 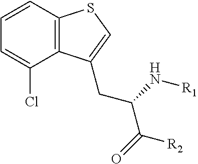 |
| 4ClW | 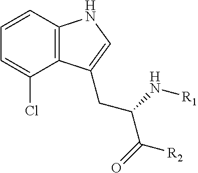 |
| 4F3COOHF | 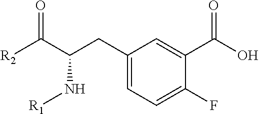 |
| 4FW | 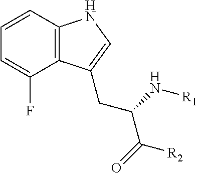 |
| 4pentenyl | 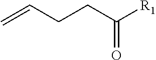 |
| 4SEF | 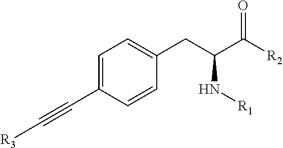 |
| 4TzF | 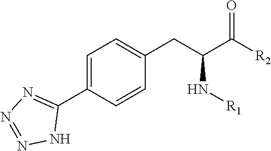 |
| 5F3Me3COOHF | 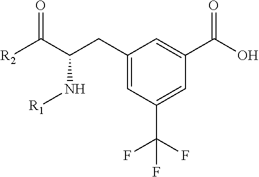 |
| 5hexenyl | 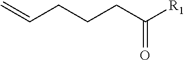 |
| 5IndA | 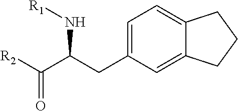 |
| 5iPr3COOHF | 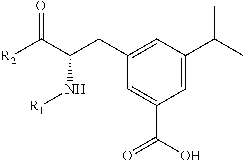 |
| 7AzaW | 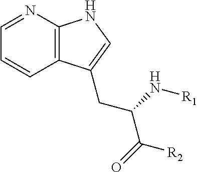 |
| 7ClBztA | 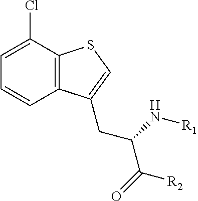 |
| 7FBztA | 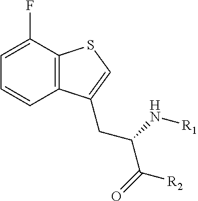 |
| AcAsp | 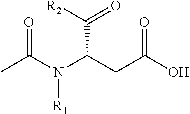 |
| AcLys | 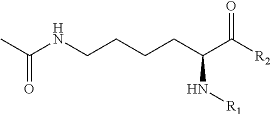 |
| AspE | 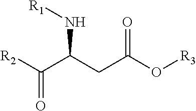 |
| AspSH | 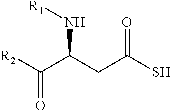 |
| Az2 | 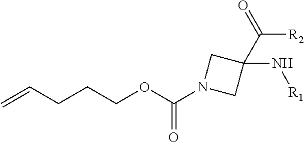 |
| Az3 | 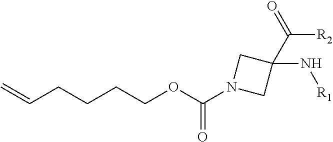 |
| B3 | 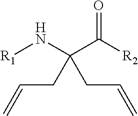 |
| B4 | 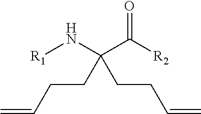 |
| B6 | 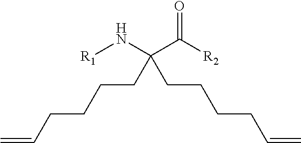 |
| bMe2Asp | 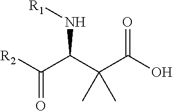 |
| Bn30Allyl | 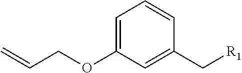 |
| BnBoroleK | 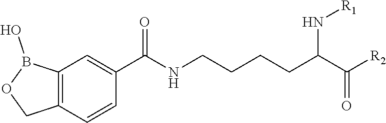 |
| Bnc | 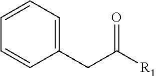 |
| BrAc | 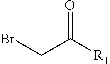 |
| BzAm2Allyl | 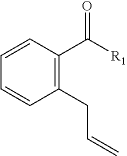 |
| BzAm3Oallyl | 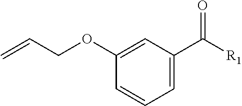 |
| Cba | 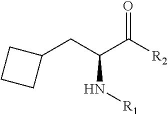 |
| Cbg | 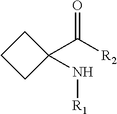 |
| ClAc | 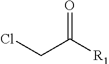 |
| CO2Bu | 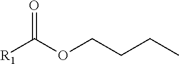 |
| CO2Hex | 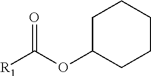 |
| CO2iBu | 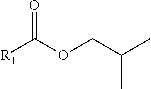 |
| CO2Me | 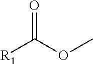 |
| CO2Ph | 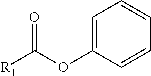 |
| Cpg | 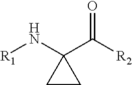 |
| CyLeu | 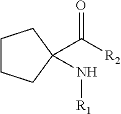 |
| dAla | 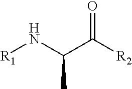 |
| dIle | 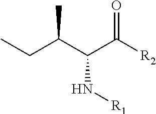 |
| dLeu | 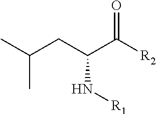 |
| F2PipAbu | 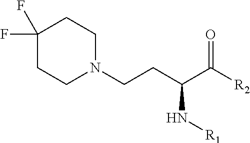 |
| F2PipNva | 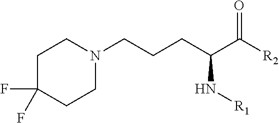 |
| GA | 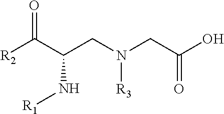 |
| GAbu | 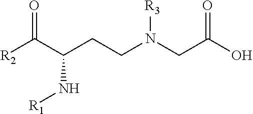 |
| GlnR | 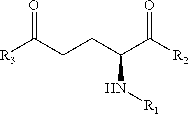 |
| GluE | 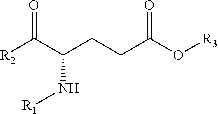 |
| GluSH | 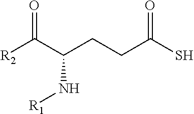 |
| hhLeu | 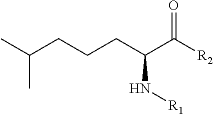 |
| HypBzEs3OAllyl | 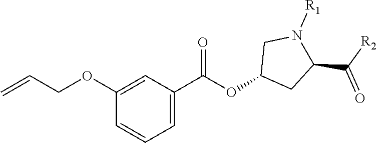 |
| HypEs4 | 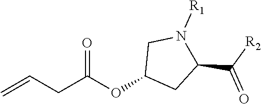 |
| HypEs5 | 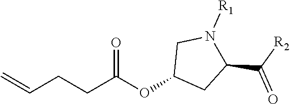 |
| HypPAc3OAllyl | 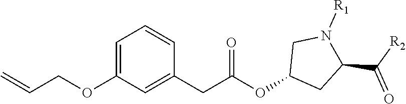 |
| Me2Asn | 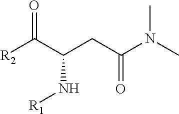 |
| Me2Gln | 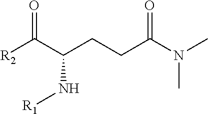 |
| MeAsn | 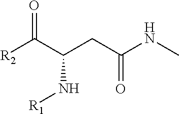 |
| MeGln | 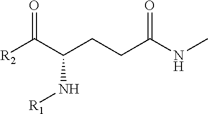 |
| MePpzAbu | 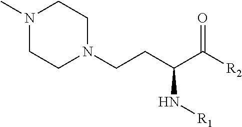 |
| MePpzAsn | 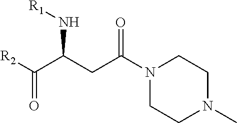 |
| MePpzNva | 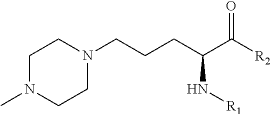 |
| MePro | 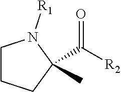 |
| Met2O | 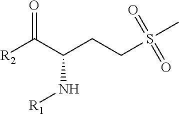 |
| MorphAbu | 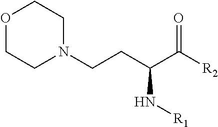 |
| MorphAsn | 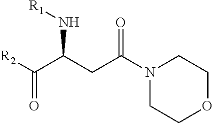 |
| MorphGln | 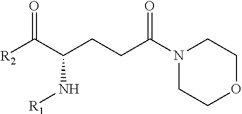 |
| MorphNva | 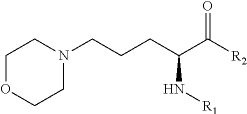 |
| NMebAla | 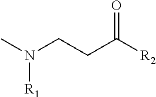 |
| Npa | 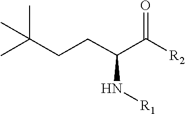 |
| PAc3OAllyl | 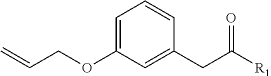 |
| ProAm5 | 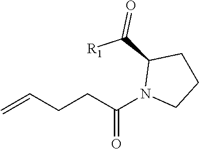 |
| ProAm6 | 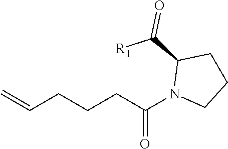 |
| ProBzAm3OAllyl | 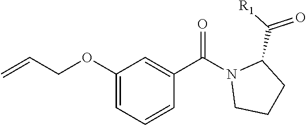 |
| ProPAc3OAllyl | 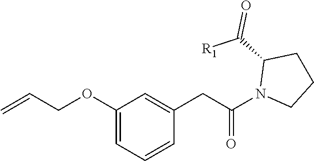 |
| PropynOH | 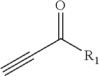 |
| ProSAm3 | 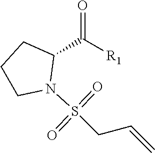 |
| PyrR | 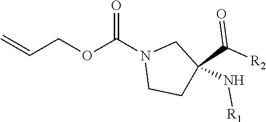 |
| PyrR2 | 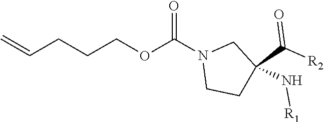 |
| PyrS4 | 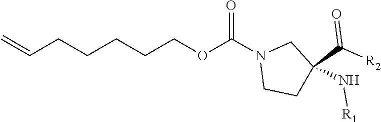 |
| R2COOPipA | 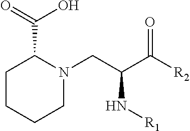 |
| R3COOPipA | 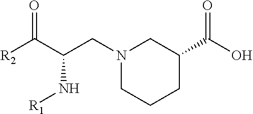 |
| RbMe2NapA | 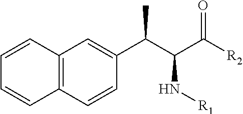 |
| RbMeBztA | 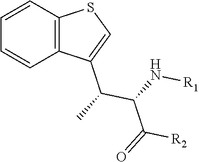 |
| RbOHAsp | 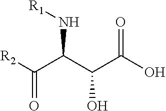 |
| S2COOPipA | 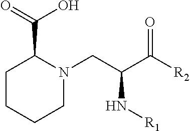 |
| S3COOPipA | 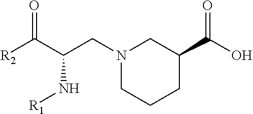 |
| sAla | 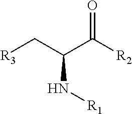 |
| Sar | 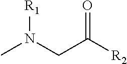 |
| SbMe2NapA | 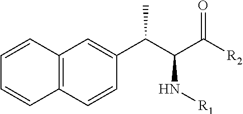 |
| SbMeBztA | 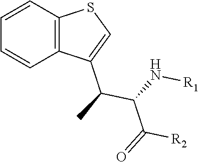 |
| SeNc5 | 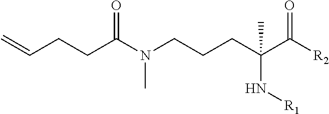 |
| SPip1 | 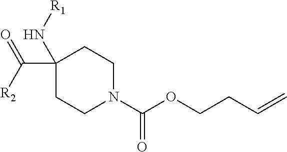 |
| SPip2 | 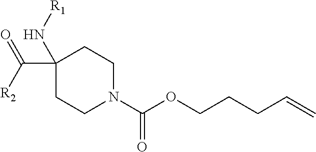 |
| SPip3 | 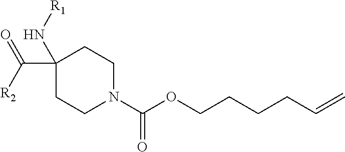 |
| ThioPro | 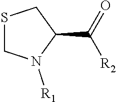 |
| TriAzDap | 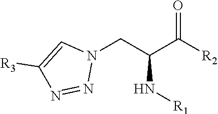 |
| TriAzDab | 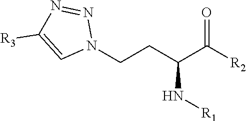 |
| TriAzLys | 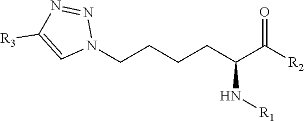 |
| TriAzdLys | 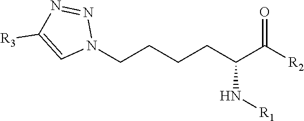 |

In some embodiments, a compound has a structure selected from the table above, wherein R₁ is —OH. In some embodiments, a compound has a structure selected from the table above, wherein R₁ is an amino protection group, e.g., Fmoc, tBoc, etc. In some embodiments, a compound has a structure selected from the table above, wherein R₁ is an amino protecting group, e.g., Fmoc, tBoc, etc., and R₂ is —OH, or —COR₂ is an optionally substituted, protected or activated carboxyl group. In some embodiments, R₂ is —OH. In some embodiments, an amino acid residue has a structure selected from the table above, wherein each of R₁ and R₂ independently represents a connection site (e.g., for structure

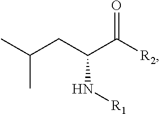

the residue is of the structure

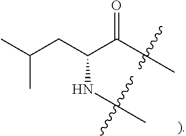 [SMILES: *N[C@H](CC(C)C)C(*)=O]

In some embodiments, an agent, a peptide or a stapled peptide comprises such an amino acid residue.

In some embodiments, a peptide comprises one or more residues of amino acids selected from the Table above. In some embodiments, a peptide comprises one or more residues of TfeGA. In some embodiments, a peptide comprises one or more residues of 2COOHF. In some embodiments, a peptide comprises one or more residues of 3COOHF.

Among other things, the present disclosure provides peptides, including stapled peptides, comprising residues of amino acids described herein. In some embodiments, the present disclosure provides various methods comprising utilizing amino acids, optionally protected and/or activated, as described herein. In some embodiments, the present disclosure provides methods for preparing peptides, comprising utilizing amino acids, typically protected and/or activated, as described herein. For example, in some embodiments, various amino groups are Fmoc protected for peptide synthesis (particularly for forming backbone peptide bonds). In some embodiments, various side chain carboxylic acid groups are t-Bu protected (—C(O)—O-tBu).

In some embodiments, the present disclosure provides methods, comprising replacing one or more acidic amino acid residues, e.g., Asp, Glu, etc., in a first compound, each independently with a provided amino acid residue, e.g., TfeGA, 2COOHF, 3COOHF, etc., to provide a second compound. In some embodiments, each of the first and second compounds is independently or independently comprises a peptide. In some embodiments, a second compound provides improved properties and/or activities (e.g., lipophilicity, Log D, etc.) compared to a first compound. In some embodiments, a second compound provides, in addition to improved properties such as lipophilicity, one or more comparable or improved other properties and/or activities (e.g., solubility and/or target binding) compared to a first compound.

In some embodiments, an agent, e.g., a peptide, a stapled peptide, a stitched peptide, etc., is less than about 5000 Daltons in mass. In some embodiments, an agent is greater than or equal to about 900 Daltons and less than about 5000 Daltons in mass. In some embodiments, an agent is greater than or equal to about 1500 Daltons and less than about 5000 Daltons in mass. In some embodiments, an agent is greater than or equal to about 2000 Daltons and less than about 5000 Daltons in mass. In some embodiments, an agent is greater than or equal to about 2500 Daltons and less than about 5000 Daltons in mass. In some embodiments, an agent is greater than or equal to about 1000 Daltons and less than about 3000 Daltons in mass. In some embodiments, an agent is greater than or equal to about 1500 Daltons and less than about 3000 Daltons in mass. In some embodiments, an agent is greater than or equal to about 1500 Daltons and less than about 2500 Daltons in mass. In some embodiments, an agent is greater than or equal to about 1600 Daltons and less than about 2200 Daltons in mass. In some embodiments, the agent is no more than about 900 Daltons in mass. In some embodiments, an agent is no more than about 500 Daltons in mass. In some embodiments, an agent is no more than about 300 Daltons in mass. In some embodiments, an agent is no more than about 200 Daltons in mass.

### Characterization

In some embodiments, agents, e.g., peptides, are characterized with respect to, for example, one or more characteristics such as binding characteristics—e.g., with respect to a particular target of interest (e.g., beta-catenin or a portion thereof), stability characteristics, for example in solution or in dried form, cell permeability characteristics, solubility, lipophilicity, etc.

In some embodiments, a binding characteristic may be or comprise specificity, affinity, on-rate, off-rate, etc, optionally under (or over a range of) specified conditions such as, for example, concentration, temperature, pH, cell type, presence or level of a particular competitor, etc.

As will be appreciated by those skilled in the art, assessments of characteristics as described herein may involve comparison with an appropriate reference (e.g., a positive or negative control) which may, in some embodiments, be a contemporaneous reference or, in some embodiments, a historical reference.

In some embodiments, desirable characteristics may be, for example: binding to a desired target (e.g., a dissociation constant (K_{D}) of at least less than about 1 μM, and preferably a K_{D} of less than about 50 nM); cell penetration (e.g., as measured by fluorescence-based assays or mass spectrometry of cellular fractions, etc.); solubility (e.g., soluble at less than about 1000 uM agent, or soluble at less than about 500 uM agent, or soluble at less than about 100 uM agent, or less than about 50 uM, or less than about 35 uM); activity (e.g., modulating one or more functions of a target, which may be assessed in a cellular reporter assay (e.g., with an IC50 of less than a concentration, e.g., less than about 1 μM, less than about 500 nM, less than about 50 nM, less than about 10 nM, etc.), an animal model (e.g., various animal models for conditions, disorders or diseases, e.g., mouse melanoma models Braf^{V600E}/Pten^{-/-} and Braf^{V600E}/Pten^{-/-}/CAT-STA) and/or a subject; stability, which may be assessed using a number of assays (e.g., in a rat pharmacokinetic study (e.g., administered via oral, iv, ip, etc.) with a terminal half-life of greater than a suitable time, e.g., 1 hour); low toxicity, which might be assessed by a number of assays (e.g., a standard ADME/toxicity assays); and/or low levels of cytotoxicity (e.g., low levels of lactate dehydrogenase (LDH) released from cells when treated at a suitable concentration, e.g., about 10 μM of a peptide). In some embodiments, an agent of the invention comprises an affinity of less than about 10 nM, for example, an IC50 of 7 nM).

In some embodiments, provided agents can bind to targets, e.g., beta-catenin, with an EC50 of no more than about 2000 nM. In some embodiments, an EC50 is no more than about 1500 nM. In some embodiments, an EC50 is no more than about 1000 nM. In some embodiments, an EC50 is no more than about 500 nM. In some embodiments, an EC50 is no more than about 300 nM. In some embodiments, an EC50 is no more than about 200 nM. In some embodiments, an EC50 is no more than about 100 nM. In some embodiments, an EC50 is no more than about 75 nM. In some embodiments, an EC50 is no more than about 50 nM. In some embodiments, an EC50 is no more than about 25 nM. In some embodiments, an EC50 is no more than about 10 nM. In some embodiments, an EC50 is no more than about 5 nM. In some embodiments, an EC50 is measured by fluorescence polarization as described in the Examples.

In some embodiments, the present disclosure provides agents, e.g., stapled peptides, with suitable solubility for various purposes. In some embodiments, solubility of provided agents, e.g., in PBS, is about or at least about 5-100 uM (e.g., about or at least about 5, 10, 15, 20, 25, 30, 40, 50, 60, 70, 80, 90, or 100 uM). In some embodiments, solubility is about or at least about 25 uM. In some embodiments, solubility is about or at least about 30 uM. In some embodiments, solubility is about or at least about 40 uM. In some embodiments, solubility is about or at least about 50 uM. In some embodiments, provided agents, e.g., stapled peptides, are protein bound in serum; in some embodiments, they are at least about 85%, 90%, or 95% protein bound in serum. In some embodiments, provided agents are over 95% protein bound in serum.

In some embodiments, provided agents can traverse a cell membrane of an animal cell. In some embodiments, provided agents can traverse a cell membrane of a human cell.

Among other things, provided agents can bind to motifs, residues, or polypeptides. In some embodiments, provided agents bind to beta-catenin. In some embodiments, a dissociation constant (K_{D}) is about 1 nM to about 1 uM. In some embodiments, a K_{D} is no more than about 1 uM. In some embodiments, a K_{D} is no more than about 500 nM. In some embodiments, a K_{D} is no more than about 250 nM. In some embodiments, a K_{D} is no more than about 100 nM. In some embodiments, a K_{D} is no more than about 50 nM. In some embodiments, a K_{D} is no more than about 25 nM. In some embodiments, a K_{D} is no more than about 10 nM. In some embodiments, a K_{D} is no more than about 5 nM. In some embodiments, a K_{D} is no more than about 1 nM. As appreciated by those skilled in the art, various technologies are available and can be utilized to measure K_{D} in accordance with the present disclosure. In some embodiments, K_{D} is measured by Surface Plasmon Resonance (SPR) as illustrated herein.

In some embodiments, provided agents binds to a polypeptide whose sequence is or comprising SEQ ID NO: 2, or a fragment thereof:

(SEQ ID NO: 2)
SVLFYAITTLHNLLLHQEGAKMAVRLAGGLQKMVALLNKTNVKFLAITTD
CLQILAYGNQESKLIILASGGPQALVNIMRTYTYEKLLWTTSRVLKVLSV
CSSNKPAIVEAGGMQALGLHLTDPSQRLVQNCLWTLRNLSDAATKQEGME
GLLGTLVQLLGSDDINVVTCAAGILSNLTCNNYKNKMMVCQVGGIEALVR
T.

In some embodiments, provided agents have one or more or all of the following interactions with beta-catenin:

Direct interactions (Italic), water mediated (bold), non-polar contacts underlined

(SEQ ID NO: 3)
LQILAYGNQESKLIILA (residue 301-317 of Uniprot
P35222 sequence)
(SEQ ID NO: 4)
SRVLKVLSVCSSN (residue 341-353 of Uniprot P35222
sequence)
(SEQ ID NO: 5)
RLVQNCLWTLRNLSDA (residue 376-391 of Uniprot
P35222 sequence)
(SEQ ID NO: 6)
LGSDDINVVTCAAGI (residue 409-423 of Uniprot P35222
sequence)

In some embodiments, an agent, e.g., a peptide, binds to beta-catenin and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 1 at the indicated positions: A305, Y306, G307, N308, Q309, K312, K345, V346, V349, Q379, N380, L382, W383, R386, N387, D413, N415, V416, T418, and C419. In some embodiments, an agent, e.g., a peptide, binds to beta-catenin and interacts with one or more residues that are or correspond to at least two, or at least three, or at least four, or at least five, or at least six, or seven of the following amino acid residues in SEQ ID NO: 1 at the indicated positions: G307, K312, K345, W383, R386, N387, D413, and N415. In some embodiments, an agent, e.g., a peptide, binds to beta-catenin and interacts with one or more residues that are or correspond to at least two, or at least three, or at least four, or at least five, or at least six, or seven of the following amino acid residues in SEQ ID NO: 1 at the indicated positions: G307, K312, K345, W383, N387, D413, and N415.

In some embodiments, provided agents interact with beta-catenin at one or more (e.g., 1, 2, 3, 4, 5, 6, or 7) of G307, K312, K345, W383, R386, N387, D413 and N415. In some embodiments, provided agents interact with beta-catenin at one or more (e.g., 1, 2, 3, 4, 5, 6, or 7) of G307, K312, K345, W383, N387, D413 and N415. In some embodiments, provided agents interact with beta-catenin at one or both of K312 and R386. In some embodiments, provided agents interact with G307. In some embodiments, provided agents interact with K312. In some embodiments, provided agents interact with beta-catenin at one or more of K345, W383, D413 and N415. In some embodiments, provided agents interact with beta-catenin at one or more of K345 and W383. In some embodiments, provided agents interact with beta-catenin at one or more of D413 and N415. In some embodiments, provided agents interact with K312. In some embodiments, provided agents interact with K345. In some embodiments, provided agents interact with W383. In some embodiments, provided agents interact with R386. In some embodiments, provided agents interact with D413. In some embodiments, provided agents interact with N415.

In some embodiments, provided agents interact with one or more of amino acid residues that are or correspond to K312, R386, K345 and W383 of SEQ ID NO: 1. In some embodiments, provided agents interact with one or more of amino acid residues that are or correspond to K312 and R386 of SEQ ID NO: 1. In some embodiments, interaction with an amino acid residue can be assessed through mutation of such an amino acid residue (e.g., mutation of K, R, etc. to D, E, etc.).

As those skilled in the art reading the present disclosure will appreciate, in some embodiments, interactions with beta-catenin may be assessed by contacting an agent with either a full-length or a portion of beta-catenin. In some embodiments, a portion of beta-catenin comprises the interacting residues above. In some embodiments, a portion of beta-catenin is or comprises SEQ ID NO: 2. In some embodiments, a portion of beta-catenin is expressed with a tag (e.g., for purification, detection, etc.). In some embodiments, a tag is a fluorescent tag. In some embodiments, a tag is for detection. In some embodiments, a tag is for purification and detection. In some embodiments, a tag is a purification tag. In some embodiments, a tag is or comprises biotin. Many other types of tags are available in the art and can be utilized in accordance with the present disclosure.

Various technologies can be utilized for characterizing and/or assessing provided technologies (e.g., agents (e.g., various peptides), compositions, methods, etc.) in accordance with the present disclosure. As described herein, in some embodiments, a useful technology is or comprises fluorescence polarization. In some embodiments, a useful technology assesses Log P or Log D. In some embodiments, a useful technology is or comprises a CHI Log D assay. In some embodiments, a useful technology assesses solubility. In some embodiments, a useful technology is or comprises NanoBRET. In some embodiments, a useful technology is or comprises a reporter assay (e.g., DLD1 reporter assay). In some embodiments, a useful technology is or comprises alphascreen. Certain useful protocols are described in the Examples. Those skilled in the art appreciate that suitable adjustments may be made to such protocols, e.g., according to specific conditions, agents, purposes, etc.

### Production

Various technologies are known in the art for producing provided agents. For example, various technologies for preparing small molecules, peptides (including stapled peptides) may be utilized in accordance with the present disclosure. Those skilled in the art, reading the present disclosure will well appreciate which such technologies are applicable in which aspects of the present disclosure in accordance with the present disclosure.

In some embodiments, as described herein, certain stapled peptides, and in particular cysteine stapled peptides, may be provided in and/or produced by a biological system and reacting with a provided reagent, e.g., one having the structure of R^{x}-L^{s2}-R^{x}, or a salt thereof, wherein R can react with —SH groups under suitable conditions. In some embodiments, each Rₓ is a suitable leaving group. In some embodiments, each R^{x} is independently —Br.

In some embodiments, peptides are prepared on solid phase on a synthesizer using, typically, Fmoc chemistry. In some embodiments, the present disclosure provides protected and/or activated amino acids for synthesis.

In some embodiments, staples are formed by olefin metathesis. In some embodiments, a product double bond of metathesis is reduced/hydrogenated. In some embodiments, CO₂ are extruded from a carbamate moiety of a staple. In some embodiments, provided stapled peptides are further modified, and/or conjugated to other entities. Conditions and/or reagents of these reactions are widely known in the art and can be performed in accordance with the present disclosure to provide stapled peptides.

Properties and/or activities of provided stapled peptides can be readily assessed in accordance with the present disclosure, for example, through use of one or more methods described in the examples.

In some embodiments, technologies for preparing and/or assessing provided stapled peptides include those described in U.S. Pat. No. 9,617,309, US 2015-0225471, US 2016-0024153, US 2016-0215036, US2016-0244494, WO 2017/062518, etc.

In some embodiments, a provided compound, e.g., an amino acid or a protected form thereof, may be prepared utilizing the following technologies.

In some embodiments, a provide compound may be prepared using one or more or all steps described below:

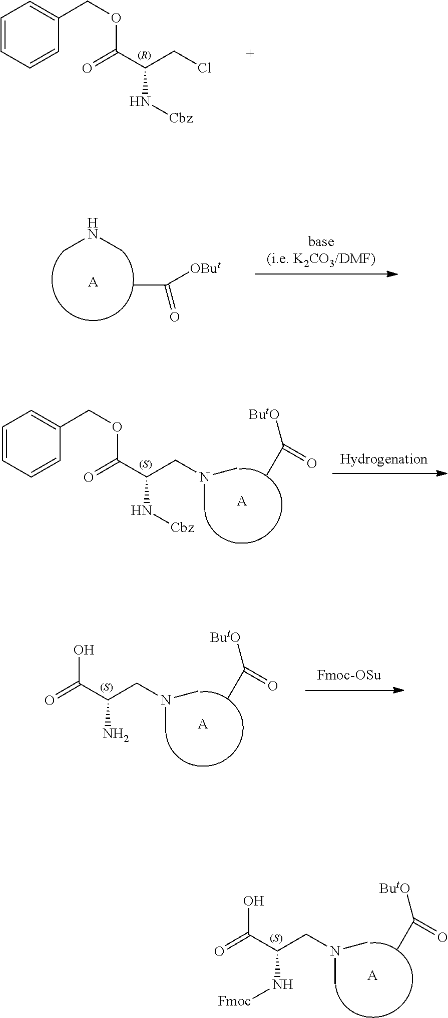

Those skilled in the art will appreciate that other leaving groups can be utilized in place of —Cl for the first reaction, such as —Br, —I, —OTs, Oms, etc.

In some embodiments, a provide compound may be prepared using one or more or all steps described below:

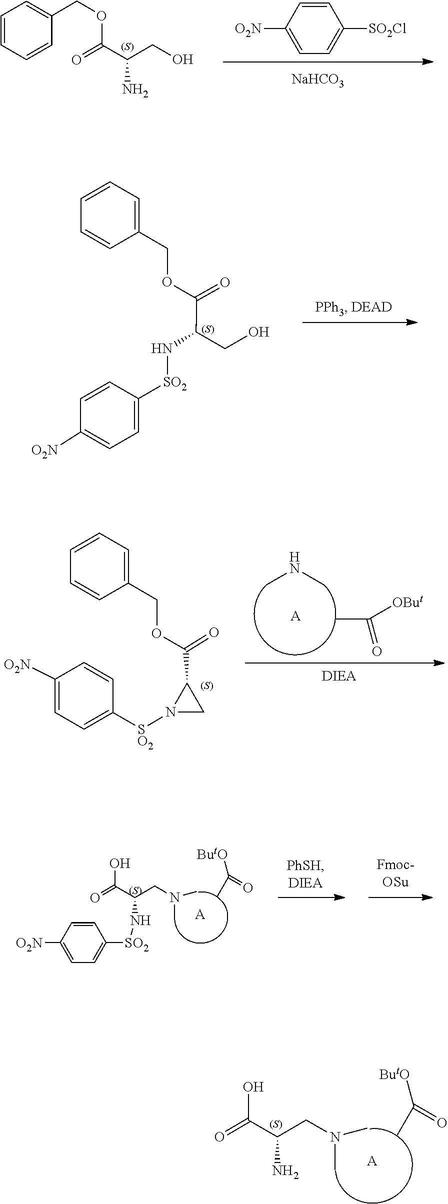

In some embodiments, a provide compound may be prepared using one or more or all steps described below:

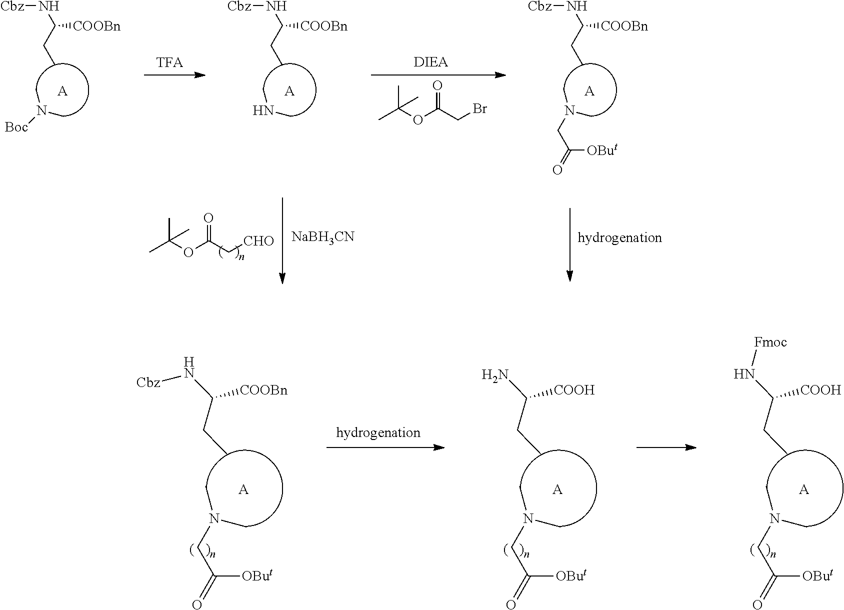

In some embodiments, a provide compound may be prepared using one or more or all steps described below:

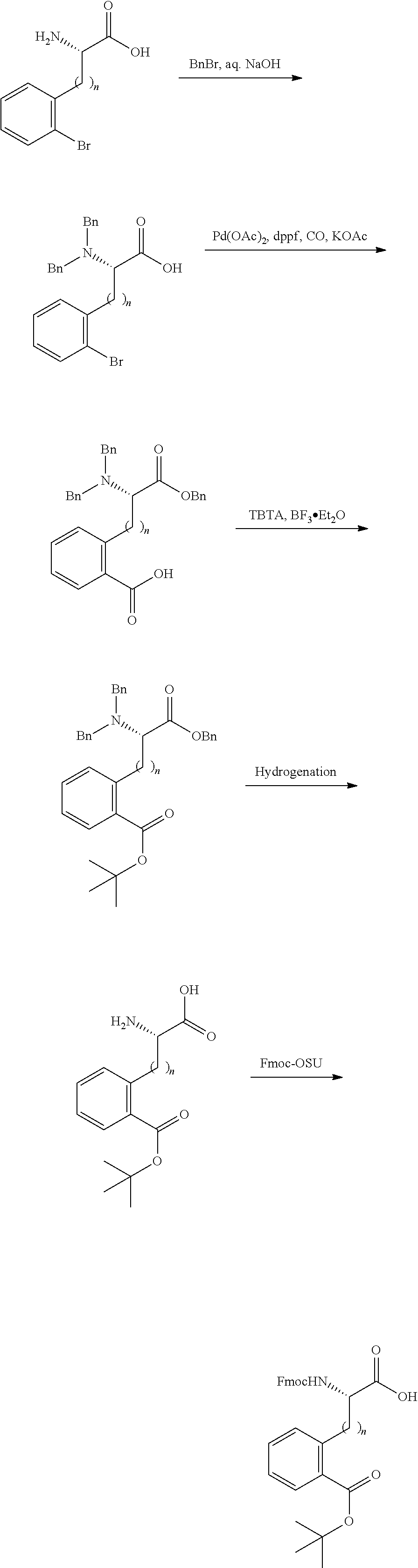

In some embodiments, a provide compound may be prepared using one or more or all steps described below:

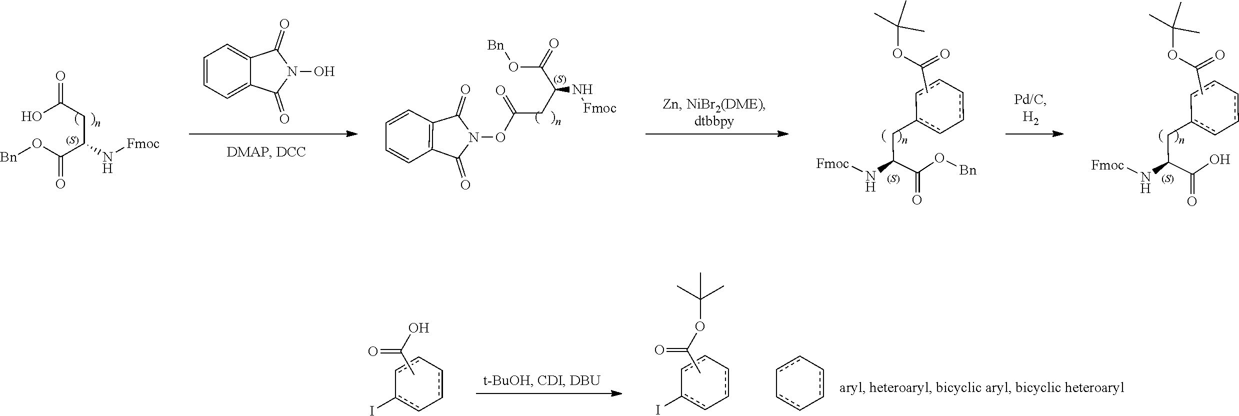

Provided compounds can be provided in high purity. In some embodiments, a provided compound is at least about 85%, 90%, 91%, 92%, 93%, 94%, 95%, 96%, 97%, 98%, or 99% pure. In some embodiments, provided compounds, e.g., amino acids optionally protected/activated, are essentially free of impurities, including stereoisomers.

### Compositions

Among other things, the present disclosure provides compositions that comprise or otherwise relate to provided agents, e.g., small molecule agents, peptide agents (e.g., stapled peptides), as described herein.

In some embodiments, provided compositions are or comprise an assay system for characterizing (and optionally including) a stapled peptide as described herein.

In some embodiments, provided compositions are pharmaceutical compositions e.g., that comprise or deliver one or more provided agents.

In some embodiments, an agent is a peptide. In some embodiments, an agent is a stapled peptide. In some embodiments, an agent comprises a detectable moiety, e.g., fluorescent moiety, radioactive moiety, biotin, etc. In some embodiments, a detectable moiety is directly detectable. In some embodiments, a detectable antibody is detected indirectly, e.g., utilizing an antibody, an agent that can reacting with a detectable moiety to form a detectable product, etc.

In some embodiments, a pharmaceutical composition comprises a provided agent and a pharmaceutically acceptable excipient (e.g., carrier).

In some embodiments, a peptide composition may include or deliver a particular form (e.g., a particular optical isomer, diastereomer, salt form, covalent conjugate form [e.g., covalently attached to a carrier moiety], etc., or combination thereof) of an agent as described herein). In some embodiments, an agent included or delivered by a pharmaceutical composition is described herein is not covalently linked to a carrier moiety.

In some embodiments, a provided therapeutic composition may comprise one or more additional therapeutic agents and/or one or more stabilizing agents and/or one or more agents that alters (e.g., extends or limits to a particular tissue, location or site) rate or extent of delivery over time.

In some embodiments, a composition is a pharmaceutical composition which comprises or delivers a provided agent (e.g., a stapled peptide) or a pharmaceutically acceptable salt thereof and a pharmaceutically acceptable excipient. In some embodiments, a composition comprises one and only stereoisomer of an agent (e.g., a stapled peptide) and/or one or more salts thereof. In some embodiments, a composition comprises two or more stereoisomers of an agent (e.g., a stapled peptide) and/or one or more salts thereof. In some embodiments, the two or more stereoisomers of an agent (e.g., a stapled peptide) or salts thereof elute as a single peak in a chromatography, e.g., HPLC.

### Uses and Applications

Provided agents and compositions can be utilized for various purposes. For example, certain compounds may be utilized as amino acids, either directly or for preparation of other compounds such as peptides. Certain agents, e.g., peptides, may be utilized to prepare stapled peptides. Certain agents that are or comprise peptides, particularly stapled peptides, and compositions thereof, are biologically active and can be utilized for various purposes, e.g., as therapeutics toward various conditions, disorders or diseases, as tools for modulating biological functions, etc.

In some embodiments, the present disclosure provides agents and compositions thereof for modulating beta-catenin functions. In some instances, beta-catenin is reported to have multiple cellular functions including regulation and coordination of cell-cell adhesion and gene transcription. In some embodiments, agents described herein may inhibit beta-catenin activity and/or level and may, for example, inhibit neoplastic growth. In some embodiments, agents described herein may activate and/or increase level of beta-catenin and may, for example, be used to treat male pattern baldness or alopecia.

It is reported that beta-catenin can interact with members of the TCF/LEF family at a TCF site on beta-catenin. In some embodiments, provided technologies can decrease, suppress or block one or more of such interactions. In some embodiments, the present disclosure provides methods for modulating an interaction between beta-catenin and its binding partner (e.g., a TCF/LEF family member) comprising contacting beta-catenin with a provided agent.

In some embodiments, binding of provided agents to beta-catenin competes or inhibits binding of another agent. In some embodiments, binding of provided agents to beta-catenin competes or inhibits binding of another agent. In some embodiments, binding of provided agents to beta-catenin competes or inhibits binding of TCF or a fragment thereof.

In some embodiments, provided agents compete with TCF7, LEF1, TCF7L1, TCF7L2, Axin1, Axin2, APC, CDH1, or CDH2, or a fragment thereof, for beta-catenin binding.

In some embodiments, provided agents interfere with interactions of TCF7, LEF1, TCF7L1, TCF7L2, Axin1, Axin2, APC, CDH1, or CDH2, or a fragment thereof, with beta-catenin.

In some embodiments, the present disclosure provides methods for modulating interactions of beta-catenin with a partner, e.g., TCF7, LEF1, TCF7L1, TCF7L2, Axin1, Axin2, APC, CDH1, or CDH2, or a fragment thereof, comprising contacting beta-catenin with a provided agent or a composition that comprises or delivers a provided agent. In some embodiments, the present disclosure provides methods for modulating interactions of beta-catenin with a partner, e.g., TCF7, LEF1, TCF7L1, TCF7L2, Axin1, Axin2, APC, CDH1, or CDH2, or a fragment thereof, comprising administering or delivering to a system comprising beta-catenin and the partner a provided agent or a composition that comprises or delivers a provided agent. In some embodiments, a system is an intro system. In some embodiments, a system is or comprises a cell, tissue or organ. In some embodiments, a system is a subject. In some embodiments, the present disclosure provides method for inhibiting cell growth, comprising administering or delivering to a population of cells an effective amount of a provided agent or a pharmaceutically acceptable salt thereof. In some embodiments, the present disclosure provides method for killing cells associated with a condition, disorder or disease (e.g., cancer), comprising administering or delivering to a population of such cells an effective amount of a provided agent or a pharmaceutically acceptable salt thereof.

In some embodiments, the present disclosure provides methods for preventing a condition, disorder or disease associated with beta-catenin (e.g., a cancer, a neurodegenerative disease, etc.), comprising administering or delivering to a subject susceptible thereto an effective amount of a provided agent or a pharmaceutically acceptable salt thereof. In some embodiments, the present disclosure provides methods for treating a condition, disorder or disease associated with beta-catenin (e.g., aberrant beta-catenin activity and/or expression level), comprising administering or delivering to a subject suffering therefrom an effective amount of a provided agent or a pharmaceutically acceptable salt thereof. In some embodiments, a provided agent is administered as a pharmaceutical composition that comprises or delivers an effective amount of a provided agent or a pharmaceutically acceptable salt thereof. In some embodiments, a condition, disorder or disease is associated with beta-catenin interaction with a partner, e.g., TCF7, LEF1, TCF7L1, TCF7L2, Axin1, Axin2, APC, CDH1, and/or CDH2. In some embodiments, a condition, disorder or disease is associated with beta-catenin with TCF. In some embodiments, a condition, disorder or disease is cancer. In some embodiments, provided agents may be administered in combination with another therapy, e.g., immunotherapy. In some embodiments, a condition, disorder, or disease is selected from cancer, cardiac disease, dilated cardiomyopathy, fetal alcohol syndrome, depression, and diabetes. In some embodiments, a condition, disorder, or disease is a heart condition, disorder, or disease. In some embodiments, a condition, disorder, or disease is cancer. In some embodiments a cancer is selected from: colon cancer, colorectal cancer, rectal cancer, prostate cancer familial adenomatous polyposis (FAP), Wilms Tumor, melanoma, hepatocellular carcinoma, ovarian cancer, endometrial cancer, medulloblastoma pilomatricomas, primary hetpatocellular carcinoma, ovarial carcinoma, breast cancer, lung cancer, glioblastoma, pliomatrixoma, medulloblastoma, thyroid tumors, and ovarian neoplasms. In some embodiments, a condition, disorder or disease is a cancer, e.g., colorectal cancer, hepatocellular cancer, melanoma, gastric cancer, bladder cancer, and endometrial cancer. In some embodiments, a cancer is colorectal cancer. In some embodiments, a cancer is hepatocellular cancer. In some embodiments, a cancer is prostate cancer. In some embodiments, a cancer is melanoma.

Various technologies can be utilized in accordance with the present disclosure to formulate, distribute, administer or deliver provided technologies such as agents, peptides, compounds, compositions, etc. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, provided technologies are administered intravenously.

Among other things, the present disclosure provides various structural moieties including designed amino acid residues that can be utilized to optimize various properties and activities, stability, delivery, pharmacodynamics, pharmacokinetics, etc. to provide various dosage forms, dosage regimen, therapeutic windows, etc. In some embodiments, provided agents and compositions thereof may be utilized with improved dosage regimen and/or unit doses. In some embodiments, administration of provided agents are adjusted based on conditions, disorders or diseases and/or subpopulations. In some embodiments, administration and/or dosage regimen of provided technologies are adjusted according to certain biomarkers and genomic alterations.

Provided agents may deliver biological effects, e.g., therapeutic effects, via various mechanisms. In some embodiments, efficacy may be driven by AUC. In some embodiments, efficacy may be driven by Cmax.

In some embodiments, a provided agent is utilized in combination with another therapy. In some embodiments, a provided agent is utilized in combination with another therapeutic agent. In some embodiments, another therapy or therapeutic agent is administered prior to an administration or delivery of a provided agent. In some embodiments, another therapy or therapeutic agent is administered at about the same time as an administration or delivery of a provided agent. In some embodiments, a provided agent and another agent is in the same pharmaceutical composition. In some embodiments, another therapy or therapeutic agent is administered subsequently to an administration or delivery of a provided agent. In some embodiments, a subject is exposed to both a provided agent and another therapeutic agent. In some embodiments, both a provided agent and another agent can be detected in a subject. In some embodiments, a provided agent is administered before another agent is cleared out by a subject or vice versa. In some embodiments, a provided agent is administered within the half-life, or 2, 3, 4, 5 or 6 times of the half-life, of another agent or vice versa. In some embodiments, a subject is exposed to a therapeutic effect of a provided agent and a therapeutic effect of another therapeutic agent. In some embodiments, an agent may provide an effect after an agent is cleared out or metabolized by a subject. In some embodiments, a procedure, e.g., surgery, radiation, etc., may provide an effect after the procedure is completed.

In some embodiments, another therapy is a cancer therapy. In some embodiments, another therapy is or comprises surgery. In some embodiments, another therapy is or comprises radiation therapy. In some embodiments, another therapy is or comprises immunotherapy. In some embodiments, another therapeutic agent is or comprises a drug. In some embodiments, another therapeutic agent is or comprises a cancer drug. In some embodiments, another therapeutic agent is or comprises a chemotherapeutic agent. In some embodiments, another therapeutic agent is or comprises a hormone therapy agent. In some embodiments, another therapeutic agent is or comprises a kinase inhibitor. In some embodiments, another therapeutic agent is or comprises a checkpoint inhibitor (e.g., antibodies against PD1-, PD-L1, CTLA-4, etc.). In some embodiments, a provide agent can be administered with lower unit dose and/or total dose compared to being used alone. In some embodiments, another agent can be administered with lower unit dose and/or total dose compared to being used alone. In some embodiments, one or more side effects associated with administration of a provided agent and/or another therapy or therapeutic agent are reduced. In some embodiments, a combination therapy provides improved results, e.g., when compared to each agent utilized individually. In some embodiments, a combination therapy achieves one or more better results, e.g., when compared to each agent utilized individually.

In some embodiments, another agent is a checkpoint inhibitor, an EGFR inhibitor, a VEGF inhibitor, a VEGFR inhibitor, a kinase inhibitor, or an anti-cancer drug.

In some embodiments, an additional agent is a checkpoint inhibitor. In some embodiments, an additional agent is an immune oncology agent. In some embodiments, an additional agent is an antibody against a checkpoint molecules. In some embodiments, an additional agent is an antibody of PD1, PDL-1, CTLA4, A2AR, B7-H3, B7-H4, BTLA, IDO, KIR, LAG3, TIM-s, C10orf54, etc. In some embodiments, an antibody is an anti-PD1 antibody. In some embodiments, an antibody is an anti-PD-L1 antibody. In some embodiments, an antibody is an anti-CTLA4.

In some embodiments, another agent is an EGFR inhibitor, e.g., erlotinib, gefitinib, lapatinib, panitumumab, vandetanib, cetuximab, etc. In some embodiments, another agent is an VEGF and/or VEGFR inhibitor, e.g., pazopanib, bevacizumab, sorafenib, sunitinib, axitinib, ponatinib, regorafenib, vandetanib, cabozantinib, ramucirumab, lenvatinib, ziv-aflibercept, etc. In some embodiments, another agent is a kinase inhibitor. In some embodiments, another therapeutic agent is a chemotherapeutic agent. In some embodiments, another therapeutic agent is an anti-cancer drug, e.g., cyclophosphamide, methotrexate, 5-fluorouracil (5-FU), doxorubicin, mustine, vincristine, procarbazine, prednisolone, dacarbazine, bleomycin, etoposide, cisplatin, epirubicin, capecitabine, folinic acid, actinomycin, all-trans retinoic acid, azacitidine, azathioprine, bortezomib, carboplatin, chlorambucil, cytarabine, daunorubicin, docetaxel, doxifluridine, fluorouracil, gemcitabine, hydroxyurea, idarubicin, imatinib, irinotecan, mechlorethamine, mercaptopurine, mitoxantrone, paclitaxel, pemetrexed, teniposide, tioguanine, topotecan, valrubicin, vinblastine, vindesine, vinorelbine, oxaliplatin, etc.

Among other things, the present disclosure provides the following Embodiments:
1. A agent having the structure of formula I:

R^{N}-L^{P1}-L^{AA1}-L^{P2}L^{AA2}-L^{P3}-L^{AA3}-L^{P4}-L^{AA4}-L^{P5}-L^{AA5}-L^{P6}-L^{AA6}-L^{P7}-R^{C} I
or a salt thereof, wherein:
   R^{N} is a peptide, an amino protecting group or R′-L^{RN}-.
   each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
      a first R′ group and a second R′ group which are taken together to form -L^{s}- which is bonded to the atom to which a first R′ group is attached and the atom to which a second R′ group is attached; and
      a third R′ group and a fourth R′ group which are taken together to form -L^{s}- which is bonded to the atom to which a third R′ group is attached and the atom to which a fourth R′ group is attached;
   each L^{s} is independently -L^{s1}-L^{s2}L^{s3}-, wherein each L^{S1}, L^{s2} and L^{s3} is independently L;
   L^{AA1} is an amino acid residue that comprises a side chain comprising an acidic or polar group;
   L^{AA2} is an amino acid residue that comprises a side chain comprising an acidic or polar group;
   L^{AA3} is an amino acid residue;
   L^{AA4} is an amino acid residue that comprises a side chain comprising an optionally substituted aromatic group;
   L^{AA5} is an amino acid residue that comprises a side chain comprising an optionally substituted aromatic group;
   L^{AA6} is an amino acid residue that comprises a side chain comprising an optionally substituted aromatic group;
   R^{C} is a peptide, a carboxyl protecting group, -L^{RC}-R′, —O-L^{RC}-R′ or —N(R′)-L^{RC}-R′;
   each of L^{RN} and L^{RC} is independently L;
   each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each -Cy- is independently an optionally substituted bivalent, 3-30 membered, monocyclic, bicyclic or polycyclic ring having 0-10 heteroatoms;
   each R′ is independently -L-R, —C(O)R, —CO₂R, or —SO₂R;
   each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
   two R groups are optionally and independently taken together to form a covalent bond, or:
   two or more R groups on the same atom are optionally and independently taken together with the atom to form an optionally substituted, 3-30 membered, monocyclic, bicyclic or polycyclic ring having, in addition to the atom, 0-10 heteroatoms; or
   two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.
2. A agent having the structure of formula I:

R^{N}-L^{P1}-L^{AA1}-L^{P2}L^{AA2}-L^{P3}-L^{AA3}-L^{P4}-L^{AA4}-L^{P5}-L^{AA5}-L^{P6}-L^{AA6}-L^{P7}-R^{C} I
or a salt thereof, wherein:
   R^{N} is a peptide, an amino protecting group or R′-L^{RN}-.
   each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
      a first R′ group and a second R′ group which are taken together to form -L^{s}- which is bonded to the atom to which a first R′ group is attached and the atom to which a second R′ group is attached; and
      a third R′ group and a fourth R′ group which are taken together to form -L^{s}- which is bonded to the atom to which a third R′ group is attached and the atom to which a fourth R′ group is attached;
   each L^{s} is independently -L^{s1}-L^{s2}-L^{s3}-, wherein each L^{s1}, L^{s2} and L^{s3} is independently L;
   L^{AA1} is L^{AR}, wherein a methylene unit is replaced with —C(R′)(R^{As})—, wherein R^{AS} is -L^{AS1}-R^{AA1} wherein R^{AA1} is —CO₂R or —SO₂R;
   L^{AA2} is L^{AR}, wherein a methylene unit is replaced with —C(R′)(R^{AS})—, wherein R^{AS} is -L^{AS2}-R^{AA2} wherein R^{AA2} is —CO₂R, or —SO₂R;
   L^{AA3} is L^{AR}, wherein a methylene unit is replaced with —C(R′)(R^{AS})—, wherein R^{AS} is -L^{AS3}-R^{AA3} wherein R^{AA3} is R′;
   L^{AA4} is L^{AR}, wherein a methylene unit is replaced with —C(R′)(R^{AS})—, wherein R^{AS} is -L^{AS4}-R^{AA4} wherein R^{AA4} is an optionally substituted group selected from 6-14 membered aryl or 5-14 membered heteroaryl having 1−6 heteroatoms;
   L^{AA5} is L^{AR}, wherein a methylene unit is replaced with —C(R′)(R^{AS})—, wherein R^{AS} is -L^{AS5}-R^{AA5}wherein R^{AA5} is an optionally substituted group selected from 6-14 membered aryl or 5-14 membered heteroaryl having 1−6 heteroatoms;
   L^{AA6} is L^{AR}, wherein a methylene unit is replaced with —C(R′)(R^{AS})—, wherein R^{AS} is -L^{AS6}R^{AA6}, wherein R^{AA6} is an optionally substituted group selected from 6-14 membered aryl or 5-14 membered heteroaryl having 1−6 heteroatoms;
   R^{C} is a peptide, a carboxyl protecting group, -L^{RC}-R′, —O-L^{RC}-R′ or —N(R′)-L^{RC}-R′;
   each of L^{RN} and L^{RC} is independently L;
   each L^{A}R is independently an optionally substituted, bivalent C₁-C₆ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each of L^{AS1}, L^{AS2}, L^{AS3}, L^{AS4}, L^{AS5}, and L^{AS6} is independently L^{AS};
   each R^{AS} is independently -L^{AS}-R′;
   each L^{AS} is independently a covalent bond or an optionally substituted, bivalent C₁-C₁₀ aliphatic or heteroaliphatic group having 1-5 heteroatoms, wherein one or more methylene units of the group optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each -Cy- is independently an optionally substituted bivalent, 3-30 membered, monocyclic, bicyclic or polycyclic ring having 0-10 heteroatoms;
   each R′ is independently -L-R, —C(O)R, —CO₂R, or —SO₂R;
   each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
   two R groups are optionally and independently taken together to form a covalent bond, or:
   two or more R groups on the same atom are optionally and independently taken together with the atom to form an optionally substituted, 3-30 membered, monocyclic, bicyclic or polycyclic ring having, in addition to the atom, 0-10 heteroatoms; or
   two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.
3. The agent of any one of the preceding Embodiments, wherein a second R′ group and a third R′ group are attached to the same atom.
4. The agent of any one of the preceding Embodiments, wherein each of the first, second and fourth R′ groups is independently attached to a different atom.
5. The agent of any one of the preceding Embodiments, wherein L^{s} formed by taking the first and the second R′ groups together is a staple as described herein.
6. The agent of any one of the preceding Embodiments, wherein L^{s} formed by taking the first and the second R′ groups together has a length of 5-20 (e.g., 5, 6, 7, 8, 9, 10, 11, 12, 13, 14, 15, 16, 17, 18, 19, or 20) atoms.
7. The agent of any one of the preceding Embodiments, wherein L^{s} formed by taking the third and the fourth R′ groups together is a staple as described herein.
8. The agent of any one of the preceding Embodiments, wherein L^{s} formed by taking the third and the fourth R′ groups together has a length of 5-20 (e.g., 5, 6, 7, 8, 9, 10, 11, 12, 13, 14, 15, 16, 17, 18, 19, or 20) atoms.
9. The agent of any one of the preceding Embodiments, wherein L^{s} formed by taking the third and the fourth R′ groups together has a length of 10-20 (e.g., 10, 11, 12, 13, 14, 15, 16, 17, 18, 19, or 20) atoms.
10. The agent of any one of the preceding Embodiments, wherein L^{P1} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
11. The agent of any one of the preceding Embodiments, wherein the length of L^{P} is 2-10 (2, 3, 4, 5, 6, 7, 8, 9, or 10) atoms.
12. The agent of any one of the preceding Embodiments, wherein one or more methylene units of L^{P1} are independently replaced with —N(R′)— or —C(O)—.
13. The agent of any one of the preceding Embodiments, wherein one or more methylene units of L^{P1} are independently replaced with —N(R′)— or —C(O)N(R′)—.
14. The agent of any one of the preceding Embodiments, wherein one or more methylene units of L^{P1} are independently replaced with —N(R′)—, —C(R′)₂, or —C(O)N(R′)—.
15. The agent of any one of the preceding Embodiments, wherein one or more methylene units of L^{P1} are independently replaced with —N(R′)—, and one or more methylene units of L^{P1} are independently replaced with —C(O)N(R′)—.
16. The agent of any one of the preceding Embodiments, wherein a methylene unit of L^{P1} is replaced with —C(R′)₂—, wherein one of the R′ groups is a first R′ group of the four R′ groups, or a methylene unit of L^{P1} is replaced with —N(R′)—, wherein the R′ group is a first R′ group of the four R′ groups.
17. The agent of any one of the preceding Embodiments, wherein a methylene unit of L^{P1} is replaced with —C(R′)₂—, wherein one of the R′ groups is a first R′ group of the four R′ groups.
18. The agent of any one of the preceding Embodiments, wherein L^{P1} is or comprises —[X]ₚ—X¹—, wherein each X and X¹ is independently an amino acid residue, wherein p is 0-10, and X¹ is bonded to L^{AA1}.
19. The agent of any one of the preceding Embodiments, wherein L^{P1} is or comprises —X¹—.
20. The agent of any one of the preceding Embodiments, wherein X¹ comprises the first R′ group of the four R′ groups.
21. The agent of any one of the preceding Embodiments, wherein L^{AA1} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
22. The agent of any one of the preceding Embodiments, wherein L^{AA1} is —N(R′)—C(R′)(R^{AS})—C(O)—.
23. The agent of any one of the preceding Embodiments, wherein L^{AA1} is NH—C(R′)(RA)C(O)—.
24. The agent of any one of the preceding Embodiments, wherein L^{AS1} is an optionally substituted, bivalent C₁-C₁₀ aliphatic group, wherein one or more methylene units of the group optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
25. The agent of any one of the preceding Embodiments, wherein L^{AS1} is an optionally substituted, bivalent C₁-C₁₀ aliphatic group, wherein one or more methylene units of the group optionally and independently replaced with —C(R′)₂—, -Cy-, —O—, —S—, —N(R′)—, —C(O)—, —S(O)—, or —S(O)₂—.
26. The agent of any one of the preceding Embodiments, wherein L^{AS1} is an optionally substituted, bivalent C₁-C₁₀ aliphatic group, wherein one or more methylene units of the group optionally and independently replaced with —O—, —S—, or —N(R′)—.
27. The agent of any one of the preceding Embodiments, wherein L^{AS1} is an optionally substituted, bivalent C₁-C₁₀ alkylene group.
28. The agent of any one of the preceding Embodiments, wherein L^{AS1} is optionally substituted —CH₂—.
29. The agent of any one of the preceding Embodiments, wherein L^{AS1} is —CH₂—.
30. The agent of any one of the preceding Embodiments, wherein R^{AA1} is —CO₂R.
31. The agent of any one of the preceding Embodiments, wherein R^{AA1} is —CO₂H.
32. The agent of any one of the preceding Embodiments, wherein L^{AA1} is an amino acid residue that comprises a side chain comprising an acidic group.
33. The agent of any one of the preceding Embodiments, wherein L^{AA1} is X².
34. The agent of any one of the preceding Embodiments, wherein L^{P2} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
35. The agent of any one of the preceding Embodiments, wherein the length of L^{P2} is 2-10 (2, 3, 4, 5, 6, 7, 8, 9, or 10) atoms.
36. The agent of any one of the preceding Embodiments, wherein the length of L^{P2} is 6 atoms.
37. The agent of any one of the preceding Embodiments, wherein one or more methylene units of L^{P2} are independently replaced with —N(R′)— or —C(O)—.
38. The agent of any one of the preceding Embodiments, wherein one or more methylene units of L^{P2} are independently replaced with —N(R′)— or —C(O)N(R′)—.
39. The agent of any one of the preceding Embodiments, wherein one or more methylene units of L^{P2} are independently replaced with —N(R′)—, —C(R′)₂, or —C(O)N(R′)—.
40. The agent of any one of the preceding Embodiments, wherein one or more methylene units of L^{P2} are independently replaced with —N(R′)—, and one or more methylene units of L^{P2} are independently replaced with —C(O)N(R′)—.
41. The agent of any one of the preceding Embodiments, wherein a methylene unit of L^{P2} is replaced with —C(R′)₂—, wherein one of the R′ groups is the second R′ group and the other is the third R′ group of the four R′ groups.
42. The agent of any one of the preceding Embodiments, wherein L^{P2} is or comprises —[X]pX⁴[X]p′-, wherein each X and X⁴ is independently an amino acid residue, and each of p and p′ is independently 0-10.
43. The agent of any one of the preceding Embodiments, wherein L^{P2} is or comprises —[X]pX³X⁴[X]p′-, wherein each X and X⁴ is independently an amino acid residue, and each of p and p′ is independently 0-10.
44. The agent of any one of the preceding Embodiments, wherein L^{P2} is or comprises —X³X⁴—, wherein each X³ and X⁴ is independently an amino acid residue, and X⁴ is bonded to L^{AA2}.
45. The agent of any one of the preceding Embodiments, wherein X⁴ comprises —C(R′)₂—, wherein one of the R′ groups is the second R′ group and the other is the third R′ group of the four R′ groups.
46. The agent of any one of the preceding Embodiments, wherein L^{s} formed by taking the first and the second R′ groups together has the structure of a L^{s} group bonded to X¹ and X⁴ as described herein.
47. The agent of any one of the preceding Embodiments, wherein L^{s} formed by taking the third and the fourth R′ groups together has the structure of a L^{s} group bonded to X⁴ and X¹ as described herein.
48. The agent of any one of the preceding Embodiments, wherein L^{AA2} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
49. The agent of any one of the preceding Embodiments, wherein L^{AA2} is —N(R′)—C(R′)(R^{AS})—C(O)—.
50. The agent of any one of the preceding Embodiments, wherein L^{AA2} is —NH—C(R′)(R^{AS})C(O)—.
51. The agent of any one of the preceding Embodiments, wherein L^{AS2} is an optionally substituted, bivalent C₁-C₁₀ aliphatic group, wherein one or more methylene units of the group optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
52. The agent of any one of the preceding Embodiments, wherein L^{AS2} is an optionally substituted, bivalent C₁-C₁₀ aliphatic group, wherein one or more methylene units of the group optionally and independently replaced with —C(R′)₂—, -Cy-, —O—, —S—, —N(R′)—, —C(O)—, —S(O)—, or —S(O)₂—.
53. The agent of any one of the preceding Embodiments, wherein L^{AS2} is an optionally substituted, bivalent C₁-C₁₀ aliphatic group, wherein one or more methylene units of the group optionally and independently replaced with —O—, —S—, or —N(R′)—.
54. The agent of any one of the preceding Embodiments, wherein L^{AS2} is an optionally substituted, bivalent C₁-C₁₀ alkylene group.
55. The agent of any one of the preceding Embodiments, wherein L^{AS2} is optionally substituted —CH₂—.
56. The agent of any one of the preceding Embodiments, wherein L^{AS2} is —CH₂—.
57. The agent of any one of the preceding Embodiments, wherein R^{AA2} is —CO₂R.
58. The agent of any one of the preceding Embodiments, wherein R^{AA2} is —C₂H.
59. The agent of any one of the preceding Embodiments, wherein L^{AA2} is an amino acid residue that comprises a side chain comprising an acidic group.
60. The agent of any one of the preceding Embodiments, wherein L^{AA2} is X⁵.
61. The agent of any one of the preceding Embodiments, wherein the length of L^{P3} is 0-10 (0, 1, 2, 3, 4, 5, 6, 7, 8, 9, or 10) atoms.
62. The agent of any one of the preceding Embodiments, wherein L^{P3} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
63. The agent of any one of the preceding Embodiments, wherein the length of L^{P3} is 0-10 (0, 1, 2, 3, 4, 5, 6, 7, 8, 9, or 10) atoms.
64. The agent of any one of the preceding Embodiments, wherein L^{P3} is a covalent bond.
65. The agent of any one of the preceding Embodiments, wherein L^{A3} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
66. The agent of any one of the preceding Embodiments, wherein L^{AA3} is —N(R′)—C(R′)(R^{AS})—C(O)—.
67. The agent of any one of the preceding Embodiments, wherein L^{AA3} is —NH—C(R′)(R^{AS})C(O)—.
68. The agent of any one of the preceding Embodiments, L^{AS3} is an optionally substituted, bivalent C₁-C₁₀ aliphatic group, wherein one or more methylene units of the group optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
69. The agent of any one of the preceding Embodiments, wherein R^{AS} is -L^{AS}-R^{AA3}, wherein L^{AS3} is an optionally substituted, bivalent C₁-C₁₀ aliphatic group, wherein one or more methylene units of the group optionally and independently replaced with —C(R′)₂—, -Cy-, —O—, —S—, —N(R′)—, —C(O)—, —S(O)—, or —S(O)₂—.
70. The agent of any one of the preceding Embodiments, wherein L^{AS3} is an optionally substituted, bivalent C₁-C₁₀ aliphatic group, wherein one or more methylene units of the group optionally and independently replaced with —O—, —S—, or —N(R′)—.
71. The agent of any one of the preceding Embodiments, wherein L^{AS3} is an optionally substituted, bivalent C₁-C₁₀ alkylene group.
72. The agent of any one of the preceding Embodiments, wherein L^{AS3} is optionally substituted —CH₂—.
73. The agent of any one of the preceding Embodiments, wherein L^{AS3} is —CH₂—.
74. The agent of any one of the preceding Embodiments, wherein R^{AA3} is —CO₂R.
75. The agent of any one of the preceding Embodiments, wherein R^{AA3} is —CO₂H.
76. The agent of any one of the preceding Embodiments, wherein L^{AA3} is an amino acid residue that comprises a side chain comprising an acidic group.
77. The agent of any one of the preceding Embodiments, wherein L^{AA3} is X⁶.
78. The agent of any one of the preceding Embodiments, wherein L^{P4} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
79. The agent of any one of the preceding Embodiments, wherein the length of L^{P4} is 2-10 (2, 3, 4, 5, 6, 7, 8, 9, or 10) atoms.
80. The agent of any one of the preceding Embodiments, wherein the length of L^{P4} is 6 atoms.
81. The agent of any one of the preceding Embodiments, wherein one or more methylene units of L^{P4} are independently replaced with —N(R′)— or —C(O)—.
82. The agent of any one of the preceding Embodiments, wherein one or more methylene units of L^{P4} are independently replaced with —N(R′)— or —C(O)N(R′)—.
83. The agent of any one of the preceding Embodiments, wherein one or more methylene units of L^{P4} are independently replaced with —N(R′)—, —C(R′)₂, or —C(O)N(R′)—.
84. The agent of any one of the preceding Embodiments, wherein one or more methylene units of L^{P4} are independently replaced with —N(R′)—, and one or more methylene units of L^{P4} are independently replaced with —C(O)N(R′)—.
85. The agent of any one of the preceding Embodiments, wherein L^{P4} is or comprises —[X]pX⁷X⁸[X]p′—, wherein each X and X¹¹ is independently an amino acid residue, and each of p and p′ is independently 0-10.
86. The agent of any one of the preceding Embodiments, wherein L^{P4} is or comprises —X⁷X⁸—, wherein each X⁷ and X⁸ is independently an amino acid residue, and ^{X} is bonded to LA4.
87. The agent of any one of the preceding Embodiments, wherein L^{A4} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
88. The agent of any one of the preceding Embodiments, wherein L^{AA4} is —N(R′)—C(R′)(R^{AS})—C(O)—.
89. The agent of any one of the preceding Embodiments, wherein L^{AA4} is —NH—C(R′)(RA)C(O)—.
90. The agent of any one of the preceding Embodiments, wherein L^{AS4} is an optionally substituted, bivalent C₁-C₁₀ aliphatic group, wherein one or more methylene units of the group optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
91. The agent of any one of the preceding Embodiments, wherein L^{AS4} is an optionally substituted, bivalent C₁-C₁₀ aliphatic group, wherein one or more methylene units of the group optionally and independently replaced with —C(R′)₂—, -Cy-, —O—, —S—, —N(R′)—, —C(O)—, —S(O)—, or —S(O)₂—.
92. The agent of any one of the preceding Embodiments, L^{AS4} is an optionally substituted, bivalent C₁-C₁₀ aliphatic group, wherein one or more methylene units of the group optionally and independently replaced with —O—, —S—, or —N(R′)—.
93. The agent of any one of the preceding Embodiments, wherein L^{AS4} is an optionally substituted, bivalent C₁-C₁₀ alkylene group.
94. The agent of any one of the preceding Embodiments, wherein L^{AS4} is optionally substituted —CH₂—.
95. The agent of any one of the preceding Embodiments, wherein L^{AS4} is —CH₂—.
96. The agent of any one of the preceding Embodiments, wherein R^{AA4} is optionally substituted 6-14 membered aryl.
97. The agent of any one of the preceding Embodiments, wherein R^{AA4} is optionally substituted phenyl.
98. The agent of any one of the preceding Embodiments, wherein R^{AA4} is phenyl.
99. The agent of any one of Embodiments 1-95, wherein R^{AA4} is optionally substituted 5-14 membered heteroaryl having 1−6 heteroatoms.
100. The agent of any one of Embodiments 1-95, wherein R^{AA4} is optionally substituted 5-membered monocyclic heteroaryl having 1-4 heteroatoms.
101. The agent of any one of Embodiments 1-95, wherein R^{AA4} is optionally substituted

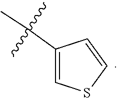 [SMILES: *c1ccsc1]

102. The agent of any one of Embodiments 1-95, wherein R^{AA4} is optionally substituted 9-membered bicyclic heteroaryl having 1-4 heteroatoms.
103. The agent of any one of Embodiments 1-95, wherein R^{AA4} is optionally substituted 10-membered bicyclic heteroaryl having 1-4 heteroatoms.
104. The agent of any one of Embodiments 1-95, wherein R^{AA4} is optionally substituted

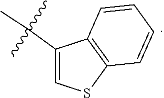 [SMILES: *c1csc2ccccc12]

105. The agent of any one of Embodiments 1-95, wherein R^{AA4} is optionally substituted

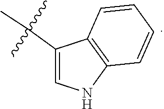 [SMILES: *c1c[nH]c2ccccc12]

106. The agent of any one of the preceding Embodiments, wherein L^{AA4} is an amino acid residue.
107. The agent of any one of the preceding Embodiments, wherein L^{AA4} is X⁹.
108. The agent of any one of the preceding Embodiments, wherein L^{P5} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
109. The agent of any one of the preceding Embodiments, wherein the length of L^{P5} is 2-10 (2, 3, 4, 5, 6, 7, 8, 9, or 10) atoms.
110. The agent of any one of the preceding Embodiments, wherein the length of L^{P5} is 6 atoms.
111. The agent of any one of the preceding Embodiments, wherein one or more methylene units of L^{P5} are independently replaced with —N(R′)— or —C(O)—.
112. The agent of any one of the preceding Embodiments, wherein one or more methylene units of L^{P5}are independently replaced with —N(R′)— or —C(O)N(R′)—.
113. The agent of any one of the preceding Embodiments, wherein one or more methylene units of L^{P5}are independently replaced with —N(R′)—, —C(R′)₂, or —C(O)N(R′)—.
114. The agent of any one of the preceding Embodiments, wherein one or more methylene units of L^{P5}are independently replaced with —N(R′)—, and one or more methylene units of L^{P5} are independently replaced with —C(O)N(R′)—.
115. The agent of any one of the preceding Embodiments, wherein a methylene unit of L^{P5} is replaced with —C(R′)₂—, wherein one of the R′ groups is the fourth R′ group.
116. The agent of any one of the preceding Embodiments, wherein L^{P5} is or comprises —[X]pX¹¹[X]p′-, wherein each X and X¹¹ is independently an amino acid residue, and each of p and p′ is independently 0-10.
117. The agent of any one of the preceding Embodiments, wherein L^{P5} is or comprises —X¹⁰X¹¹—, wherein each X¹⁰ and X¹¹ is independently an amino acid residue, and X¹¹ is bonded to L^{AA5}.
118. The agent of any one of the preceding Embodiments, wherein X¹¹ comprises —C(R′)₂—, wherein one of the R′ groups is the fourth R′ group.
119. The agent of any one of the preceding Embodiments, wherein L^{AA5} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
120. The agent of any one of the preceding Embodiments, wherein L^{AA5} is —N(R′)—C(R′)(R^{AS})—C(O)—.
121. The agent of any one of the preceding Embodiments, wherein L^{AA5} is —NH—C(R′)(RA)C(O)—.
122. The agent of any one of the preceding Embodiments, wherein L^{AS5} is an optionally substituted, bivalent C₁-C₁₀ aliphatic group, wherein one or more methylene units of the group optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
123. The agent of any one of the preceding Embodiments, wherein L^{AS5} is an optionally substituted, bivalent C₁-C₁₀ aliphatic group, wherein one or more methylene units of the group optionally and independently replaced with —C(R′)₂—, -Cy-, —O—, —S—, —N(R′)—, —C(O)—, —S(O)—, or —S(O)₂—.
124. The agent of any one of the preceding Embodiments, wherein L^{AS5} is an optionally substituted, bivalent C₁-C₁₀ aliphatic group, wherein one or more methylene units of the group optionally and independently replaced with —O—, —S—, or —N(R′)—.
125. The agent of any one of the preceding Embodiments, wherein L^{AS5} is an optionally substituted, bivalent C₁-C₁₀ alkylene group.
126. The agent of any one of the preceding Embodiments, wherein L^{AS5} is optionally substituted —CH₂—.
127. The agent of any one of the preceding Embodiments, wherein L^{AS5} is —CH₂—.
128. The agent of any one of the preceding Embodiments, wherein R^{AA5} is optionally substituted 6-14 membered aryl.
129. The agent of any one of the preceding Embodiments, wherein R^{AA5} is optionally substituted phenyl.
130. The agent of any one of the preceding Embodiments, wherein R^{AAA5} is phenyl.
131. The agent of any one of Embodiments 1-95, wherein R^{AA5} is optionally substituted 5-14 membered heteroaryl having 1−6 heteroatoms.
132. The agent of any one of Embodiments 1-95, wherein R^{AA5} is optionally substituted 5-membered monocyclic heteroaryl having 1-4 heteroatoms.

133. The agent of any one of Embodiments 1-95, wherein R^{AA5} is optionally substituted

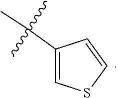 [SMILES: *c1ccsc1]

134. The agent of any one of Embodiments 1-95, wherein R^{AA5} is optionally substituted 9-membered bicyclic heteroaryl having 1-4 heteroatoms.
135. The agent of any one of Embodiments 1-95, wherein R^{AA5} is optionally substituted 10-membered bicyclic heteroaryl having 1-4 heteroatoms.
136. The agent of any one of Embodiments 1-95, wherein R^{AA5} is optionally substituted

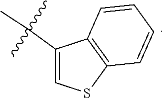 [SMILES: *c1csc2ccccc12]

137. The agent of any one of Embodiments 1-95, wherein R^{AA5} is optionally substituted

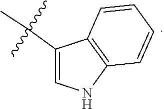 [SMILES: *c1c[nH]c2ccccc12]

138. The agent of any one of the preceding Embodiments, wherein L^{AA5} is an amino acid residue.
139. The agent of any one of the preceding Embodiments, wherein L^{AA5} is X¹².
140. The agent of any one of the preceding Embodiments, wherein L^{P6} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
141. The agent of any one of the preceding Embodiments, wherein the length of L^{P6} is 0-10 (0, 1, 2, 3, 4, 5, 6, 7, 8, 9, or 10) atoms.
142. The agent of any one of the preceding Embodiments, wherein the length of L^{P6} is a covalent bond.
143. The agent of any one of the preceding Embodiments, wherein L^{AA6} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
144. The agent of any one of the preceding Embodiments, wherein a methylene unit of L^{AA6} is replaced with —C(R′)(R^{AS})—, wherein R^{AS} is -L^{AS}-R^{AA6}, wherein L^{AS} is an optionally substituted, bivalent C₁-C₁₀ aliphatic group, wherein one or more methylene units of the group optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
145. The agent of any one of the preceding Embodiments, wherein a methylene unit of L^{AA} is replaced with —C(R′)(R^{AS})—, wherein R^{AS} is -L^{AS}-R^{AA6}, wherein L^{AS} is an optionally substituted, bivalent C₁-C₁₀ aliphatic group, wherein one or more methylene units of the group optionally and independently replaced with —C(R′)₂—, -Cy-, —O—, —S—, —N(R′)—, —C(O)—, —S(O)—, or —S(O)₂—.
146. The agent of any one of the preceding Embodiments, wherein a methylene unit of L^{AA6} is replaced with —C(R′)(R^{AS})—, wherein R^{AS} is -L^{AS}-R^{AA6}, wherein L^{AS} is an optionally substituted, bivalent C₁-C₁₀ aliphatic group, wherein one or more methylene units of the group optionally and independently replaced with —O—, —S—, or —N(R′)—.
147. The agent of any one of the preceding Embodiments, wherein a methylene unit of L^{AA6} is replaced with —C(R′)(R^{AS})—, wherein R^{AS} is -L^{AS}-R^{AA6}, wherein L^{AS} is an optionally substituted, bivalent C₁-C₁₀ alkylene group.
148. The agent of any one of the preceding Embodiments, wherein a methylene unit of L^{AA6} is replaced with —C(R′)(R^{AS})—, wherein R^{AS} is —CH₂—R^{AA6}.
149. The agent of any one of the preceding Embodiments, wherein R^{AA6} is optionally substituted 6-14 membered aryl.
150. The agent of any one of the preceding Embodiments, wherein R^{AA6} is optionally substituted phenyl.
151. The agent of any one of the preceding Embodiments, wherein R^{AA6} is phenyl.
152. The agent of any one of Embodiments 1-95, wherein R^{AA6} is optionally substituted 5-14 membered heteroaryl having 1−6 heteroatoms.
153. The agent of any one of Embodiments 1-95, wherein R^{AA6} is optionally substituted 5-membered monocyclic heteroaryl having 1-4 heteroatoms.
154. The agent of any one of Embodiments 1-95, wherein R^{AA6} is optionally substituted

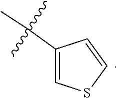 [SMILES: *c1ccsc1]

155. The agent of any one of Embodiments 1-95, wherein R^{AA6} is optionally substituted 9-membered bicyclic heteroaryl having 1-4 heteroatoms.
156. The agent of any one of Embodiments 1-95, wherein R^{AA6} is optionally substituted 10-membered bicyclic heteroaryl having 1-4 heteroatoms.
157. The agent of any one of Embodiments 1-95, wherein R^{AA6} is optionally substituted

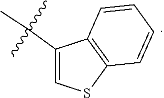 [SMILES: *c1csc2ccccc12]

158. The agent of any one of Embodiments 1-95, wherein R^{AA6} is optionally substituted

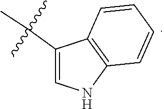 [SMILES: *c1c[nH]c2ccccc12]

159. The agent of any one of the preceding Embodiments, wherein L^{AA6} is an amino acid residue.
160. The agent of any one of the preceding Embodiments, wherein L^{AA6} is X¹³.
161. The agent of any one of the preceding Embodiments, wherein L^{P7} is a covalent bond, or an optionally substituted, bivalent C₁-C₁₀ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
162. The agent of any one of the preceding Embodiments, wherein the length of L^{P7} is 0-20 (e.g., 0-15, 0-10, 0-5, 0, 1, 2, 3, 4, 5, 6, 7, 8, 9, 10, 11, 12, 13, 14, 15, 16, 17, 18, 19, or 20) atoms.
163. The agent of any one of the preceding Embodiments, wherein L^{RN} is a covalent bond, or an optionally substituted, bivalent C₁-C₁₀ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
164. The agent of any one of the preceding Embodiments, wherein the length of L^{RN} is 0-20 (0, 1, 2, 3, 4, 5, 6, 7, 8, 9, 10, 11, 12, 13, 14, 15, 16, 17, 18, 19, or 20) atoms.
165. The agent of any one of the preceding Embodiments, wherein R^{N} is R′-L^{RN}-, wherein R′ is —C(O)R, —CO₂R, or —SO₂R.
166. The agent of any one of the preceding Embodiments, wherein R^{N} is R′, wherein R′ is —C(O)R, —CO₂R, or —SO₂R.
167. The agent of any one of the preceding Embodiments, wherein L^{RC} is a covalent bond, or an optionally substituted, bivalent C₁-C₁₀ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
168. The agent of any one of the preceding Embodiments, wherein the length of L^{RC} is 0-20 (0, 1, 2, 3, 4, 5, 6, 7, 8, 9, 10, 11, 12, 13, 14, 15, 16, 17, 18, 19, or 20) atoms.
169. The agent of any one of the preceding Embodiments, wherein R^{C} is —O-L^{RC}-R′ or —N(R′)-L^{RC}-R′.
170. The agent of any one of the preceding Embodiments, wherein R^{C} is —OR′ or —N(R′)₂, wherein each R′ is independently R.
171. The agent of any one of the preceding Embodiments, wherein the agent is or comprise a peptide.
172. The agent of any one of the preceding Embodiments, wherein the agent is a peptide.
173. The agent of any one of the preceding Embodiments, wherein the agent is or comprises a helix structure.
174. An agent, wherein the agent is or comprise a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,
wherein:
   each of p14, p15, p16 and p17 is independently 0 or 1;
   each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
   X² comprises a side chain comprising an acidic or polar group;
   X⁵ comprises a side chain comprising an acidic or polar group;
   X⁹ comprises a side chain comprising an optionally substituted aromatic group;
   X² comprises a side chain comprising an optionally substituted aromatic group; and
   X¹³ comprises a side chain comprising an optionally substituted aromatic group.
175. An agent, wherein the agent is or comprises:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,
wherein each of p14, p15, p16, p17, p18, p19, p20, p21, p22, and p23 is independently 0 or 1, and each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue.
176. An agent, wherein the agent is or comprises:

[X]pX¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X]p′;
wherein:
   each of p14, p15, p¹⁶ and p17 is independently 0 or 1;
   each of p and p′ is independently 0-10;
   each of X, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.
177. An agent, wherein the agent is or comprise a peptide comprising:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,
wherein:
   each of p14, p15, p16 and p17 is independently 0 or 1;
   each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
   X² comprises a side chain comprising an acidic or polar group;
   X⁵ comprises a side chain comprising an acidic or polar group;
   X⁹ comprises a side chain comprising an optionally substituted aromatic group;
   X² comprises a side chain comprising an optionally substituted aromatic group;
   X¹³ comprises a side chain comprising an optionally substituted aromatic group; and wherein:
   X¹ and X⁴, and/or X⁴ and X¹¹ are independently amino acid residues suitable for stapling, or are stapled, or X³ and X¹⁰ are independently amino acid residues suitable for stapling, or are stapled.
178. The agent of Embodiment 174, wherein X¹ and X⁴, and X⁴ and X¹¹ are independently amino acid residues suitable for stapling.
179. The agent of Embodiment 174, wherein X¹ and X⁴, and X⁴ and X¹¹ are independently stapled.
180. The agent of any one of Embodiments 1-173, where the agent is an agent of any one of Embodiments 174-179.
181. The agent of any one of the preceding Embodiments, comprising a staple having the structure of LS which is -L^{s1}-L^{s2}-L^{s3}-.
182. The agent of any one of the preceding Embodiments, wherein there are two staples in the agent each independently having the structure of L^{s} which is -L^{s1}-L^{s2}-L^{s3}-.
183. The agent of any one of Embodiments 181-182, wherein L^{s1} is a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
184. The agent of Embodiment 183, wherein L^{s1} is an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
185. The agent of Embodiment 183, wherein L^{s1} is an optionally substituted bivalent linear or branched, saturated or partially unsaturated C₁₋₆ hydrocarbon chain, wherein one or more methylene units are optionally and independently replaced with —O—, -Cy-, —S—, —N(R′)—, —C(O)—, —C(O)N(R′)—, or —N(R′)C(O)O—.
186. The agent of Embodiment 183, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
187. The agent of any one of Embodiments 184-186, wherein L^{s1} comprises —N(R′)—.
188. The agent of any one of Embodiments 184-186, wherein L^{s1} comprises —N(R′)C(O)O—.
189. The agent of Embodiment 188, wherein —N(R′)— is closer to L^{s2}.
190. The agent of Embodiment 188, wherein —O— is closer to L^{s2}.
191. The agent of any one of Embodiments 184-186, wherein L^{s1} is —(CH₂)m-N(R′)—(CH₂)ₙ—, wherein each of m and n is independently 1, 2, 3, 4, 5, 6, 7, 8, 9, or 10.
192. The agent of any one of Embodiments 184-186, wherein L^{s1} is —(CH₂)m-N(R′)—C(O)—O—(CH₂)ₙ—, wherein each of m and n is independently 1, 2, 3, 4, 5, 6, 7, 8, 9, or 10.
193. The agent of any one of Embodiments 191-192, wherein —(CH₂)m- is bonded L^{s2}.
194. The agent of any one of Embodiments 191-192, wherein —(CH₂)ₙ— is bonded L^{s2}.
195. The agent of any one of Embodiments 191-194, wherein m is 1.
196. The agent of any one of Embodiments 191-194, wherein m is 2.
197. The agent of any one of Embodiments 191-196, wherein n is 3.
198. The agent of any one of Embodiments 187-197, wherein R′ is —H.
199. The agent of any one of Embodiments 187-197, wherein R′ is optionally substituted C₁₋₆ aliphatic.
200. The agent of any one of Embodiments 187-197, wherein R′ is methyl.
201. The agent of any one of Embodiments 187-197, wherein R′ is taken together with R^{a3} of the amino acid residue to which L^{s1} is bonded to and their intervening atom(s) to form an optionally substituted 3-10 membered ring having 0-5 heteroatoms in addition to the intervening atom(s).
202. The agent of Embodiment 201, wherein R′ is taken together with R^{a3} of the amino acid residue to which L^{s3} is bonded to and their intervening atom(s) to form a 3-10 membered monocyclic ring having 0-5 heteroatoms in addition to the intervening atom(s).
203. The agent of any one of Embodiments 201-202, wherein the formed ring is saturated.
204. The agent of any one of Embodiments 201-203, wherein the formed ring is 4-membered.
205. The agent of any one of Embodiments 201-203, wherein the formed ring is 5-membered.
206. The agent of any one of Embodiments 201-205, wherein the formed ring has no heteroatoms in addition to the intervening atom(s).
207. The agent of Embodiment 183, wherein L^{s1} is optionally substituted —(CH₂)ₙ—, wherein n is 1, 2, 3, 4, 5, or 6.
208. The agent of Embodiment 183, wherein L^{s1} is —(CH₂)ₙ—, wherein n is 1, 2, 3, 4, 5, or 6.
209. The agent of Embodiment 183, wherein L^{s1} is —CH₂—.
210. The agent of Embodiment 183, wherein L^{s1} is optionally substituted —(CH₂)ₙ—C(O)—, wherein n is 1, 2, 3, 4, 5, or 6.
211. The agent of Embodiment 183, wherein L^{s1} is —(CH₂)ₙ—C(O)—, wherein n is 1, 2, 3, 4, 5, or 6.
212. The agent of Embodiment 183, wherein L^{s1} is —(CH₂)ₙ—C(O)—, wherein n is 2 or 3.
213. The agent of any one of Embodiments 181-212, wherein L^{s1} is bonded to an amino acid residue closer to the N-terminus than an amino acid residue to which -L^{s3}- is bond.
214. The agent of any one of Embodiments 181-213, wherein L^{s1} is bond to a carbon atom of the peptide backbone.
215. The agent of any one of Embodiments 181-214, wherein L^{s1} is bond to an alpha carbon atom of an amino acid residue.
216. The agent of any one of Embodiments 181-213, wherein L^{s1} is bond to a nitrogen atom of the peptide backbone.
217. The agent of any one of Embodiments 181-213, wherein L^{s1} is bond to a nitrogen atom of the peptide backbone, wherein the nitrogen atom is of an amino group bonded to an alpha carbon atom of an amino acid residue.
218. The agent of any one of Embodiments 216-217, wherein the nitrogen atom is bond to —C(O)— of L^{s1}.
219. The agent of any one of Embodiments 181-218, wherein L^{s2} is a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
220. The agent of any one of Embodiments 181-218, wherein L^{s2} is an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S—Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
221. The agent of Embodiment 220, wherein L^{s2} is optionally substituted —CH═CH—.
222. The agent of Embodiment 220, wherein L^{s2} is —CH═CH—.
223. The agent of Embodiment 220, wherein the double bond is E.
224. The agent of Embodiment 220, wherein the double bond is Z.
225. The agent of Embodiment 220, wherein L^{s2} is optionally substituted —CH₂—CH₂—.
226. The agent of Embodiment 220, wherein L^{s2} is —CH₂—CH₂—.
227. The agent of Embodiment 220, wherein L^{s2} is -Cy-.
228. The agent of Embodiment 227, wherein -Cy- is optionally substituted saturated or partially unsaturated 5−6 membered ring having 0-4 heteroatoms.
229. The agent of Embodiment 227, wherein -Cy- is optionally substituted phenyl ring.
230. The agent of Embodiment 227, wherein -Cy- is optionally substituted 5−6 membered aromatic ring having 1-4 heteroatoms.
231. The agent of Embodiment 227, wherein -Cy- is optionally substituted

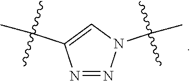 [SMILES: *c1cn(*)nn1]

232. The agent of Embodiment 227, wherein -Cy- is

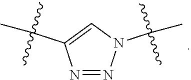 [SMILES: *c1cn(*)nn1]

233. The agent of any one of Embodiments 231-232, wherein the carbon atom is bonded to L^{s1}.
234. The agent of any one of Embodiments 231-232, wherein the carbon atom is bonded to L^{s3}.
235. The agent of Embodiment 220, wherein L^{s2} is —C(O)N(R′)—.
236. The agent of Embodiment 235, wherein R′ is —H.
237. The agent of Embodiment 235, wherein R′ is optionally substituted C₁₋₆ aliphatic.
238. The agent of any one of Embodiments 235-237, wherein the —N(R′)— is bonded to L^{s1}.
239. The agent of any one of Embodiments 235-237, wherein the —N(R′)— is bonded to L^{s3}.
240. The agent of Embodiment 220, wherein one or more methylene units are independently replaced with —C(O)N(R′)— or —N(R′)—, and one or more methylene units are independently replaced with —C(R′)₂—, wherein one or more R′ of one or more —C(R′)₂— are each independently taken together with R′ of —C(O)N(R′)— or —N(R′)— and their intervening atom(s) to form an optionally substituted 3-10 membered ring having 0-5 heteroatoms in addition to the intervening atom(s).
241. The agent of Embodiment 240, wherein the formed ring is saturated.
242. The agent of any one of Embodiments 240-241, wherein the formed ring is 4-membered.
243. The agent of any one of Embodiments 240-242, wherein the formed ring is 5-membered.
244. The agent of any one of Embodiments 240-243, wherein the formed ring has no heteroatoms in addition to the intervening atom(s).
245. The agent of Embodiment 220, wherein L^{s2} is —S-L″—S—.
246. The agent of Embodiment 245, wherein L″ is a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
247. The agent of Embodiment 245, wherein L″ is an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
248. The agent of Embodiment 245, wherein L″ is or comprise -Cy-.
249. The agent of Embodiment 245, wherein L″ is or comprise —(CH₂)m-Cy-(CH₂)ₙ—, wherein each m and n is optionally substituted 0 1, 2, 3, 4, 5, 6, 7, 8, 9, or 10, and each —CH₂— is optionally substituted.
250. The agent of Embodiment 249, wherein each m and n is independently 1.
251. The agent of any one of Embodiments 248-250, wherein is optionally substituted phenyl.
252. The agent of any one of Embodiments 248-250, wherein is optionally substituted 5−6 membered aromatic ring having 1-4 heteroatoms.
253. The agent of any one of Embodiments 181-252, wherein L^{s3} is a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
254. The agent of Embodiment 253, wherein L^{s3} is an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
255. The agent of Embodiment 253, wherein L^{s3} is an optionally substituted bivalent linear or branched, saturated or partially unsaturated C₁₋₆ hydrocarbon chain, wherein one or more methylene units are optionally and independently replaced with —O—, -Cy-, —S—, —N(R′)—, —C(O)—, —C(O)N(R′)—, or —N(R′)C(O)O—.
256. The agent of Embodiment 253, wherein L^{s3} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
257. The agent of any one of Embodiments 254-256, wherein L^{s3} comprises —N(R′)—.
258. The agent of any one of Embodiments 254-256, wherein L^{s3} comprises —N(R′)C(O)O—.
259. The agent of Embodiment 258, wherein —N(R′)— is closer to L^{s2}.
260. The agent of Embodiment 258, wherein —O— is closer to L^{s2}
261. The agent of any one of Embodiments 254-256, wherein L^{s3} is —(CH₂)m-N(R′)—(CH₂)ₙ—, wherein each of m and n is independently 1, 2, 3, 4, 5, 6, 7, 8, 9, or 10.
262. The agent of any one of Embodiments 254-256, wherein L^{s3} is —(CH₂)m-N(R′)—C(O)—O—(CH₂)ₙ—, wherein each of m and n is independently 1, 2, 3, 4, 5, 6, 7, 8, 9, or 10.
263. The agent of any one of Embodiments 261-262, wherein —(CH₂)ₙ— is bonded L^{s2}
264. The agent of any one of Embodiments 261-262, wherein —(CH₂)m- is bonded L^{s2}.
265. The agent of any one of Embodiments 261-264, wherein m is 1.
266. The agent of any one of Embodiments 261-264, wherein m is 2.
267. The agent of any one of Embodiments 261-266, wherein n is 3.
268. The agent of any one of Embodiments 257-267, wherein R′ is —H.
269. The agent of any one of Embodiments 257-267, wherein R′ is optionally substituted C₁₋₆ aliphatic.
270. The agent of any one of Embodiments 257-267, wherein R′ is methyl.
271. The agent of any one of Embodiments 257-267, wherein R′ is taken together with R^{a3} of the amino acid residue to which L^{s3} is bonded to and their intervening atom(s) to form an optionally substituted 3-10 membered ring having 0-5 heteroatoms in addition to the intervening atom(s).
272. The agent of any one of Embodiments 257-267, wherein R′ is taken together with R^{a3} of the amino acid residue to which L^{s3} is bonded to and their intervening atom(s) to form a 3-10 membered monocyclic ring having 0-5 heteroatoms in addition to the intervening atom(s).
273. The agent of any one of Embodiments 271-272, wherein the formed ring is saturated.
274. The agent of any one of Embodiments 271-273, wherein the formed ring is 4-membered.
275. The agent of any one of Embodiments 271-274, wherein the formed ring is 5-membered.
276. The agent of any one of Embodiments 271-275, wherein the formed ring has no heteroatoms in addition to the intervening atom(s).
277. The agent of Embodiment 253, wherein L^{s3} is optionally substituted —(CH₂)ₙ—, wherein n is 1, 2, 3, 4, 5, or 6.
278. The agent of Embodiment 253, wherein L^{s3} is —(CH₂)ₙ—, wherein n is 1, 2, 3, 4, 5, or 6.
279. The agent of Embodiment 253, wherein L^{s3} is —(CH₂)₃—.
280. The agent of Embodiment 253, wherein L^{s3} is —(CH₂)₂—.
281. The agent of Embodiment 253, wherein L^{s3} is —CH₂—.
282. The agent of Embodiment 253, wherein L^{s3} is optionally substituted —(CH₂)ₙ—C(O)—, wherein n is 1, 2, 3, 4, 5, or 6.
283. The agent of Embodiment 253, wherein L^{s3} is —(CH₂)ₙ—C(O)—, wherein n is 1, 2, 3, 4, 5, or 6.
284. The agent of Embodiment 253, wherein L^{s3} is —(CH₂)ₙ—C(O)—, wherein n is 2 or 3.
285. The agent of any one of Embodiments 181-284, wherein L^{s3} is bonded to an amino acid residue closer to the N-terminus than an amino acid residue to which -L^{s3}- is bond.
286. The agent of any one of Embodiments 181-285, wherein L^{s3} is bond to a carbon atom of the peptide backbone.
287. The agent of any one of Embodiments 181-286, wherein L^{s3} is bond to an alpha carbon atom of an amino acid residue.
288. The agent of any one of Embodiments 181-285, wherein L^{s3} is bond to a nitrogen atom of the peptide backbone.
289. The agent of any one of Embodiments 181-285, wherein L^{s3} is bond to a nitrogen atom of the peptide backbone, wherein the nitrogen atom is of an amino group bonded to an alpha carbon atom of an amino acid residue.
290. The agent of any one of Embodiments 288-289, wherein the nitrogen atom is bond to —C(O)— of L^{s3}.
291. The agent of any one of Embodiments 181-290, wherein a staple is optionally substituted —CH₂—CH═CH—(CH₂)₃—.
292. The agent of any one of Embodiments 181-290, wherein a staple is —CH₂—CH═CH—(CH₂)₃—.
293. The agent of Embodiment 291-292, wherein —CH═CH— is E.
294. The agent of Embodiment 291-292, wherein —CH═CH— is Z.
295. The agent of any one of Embodiments 181-290, wherein a staple is optionally substituted —CH₂—CH═CH—(CH₂)₃—C(O)—.
296. The agent of any one of Embodiments 181-290, wherein a staple is —CH₂—CH═CH—(CH₂)₃—C(O)—.
297. The agent of any one of Embodiments 181-290, wherein a staple is optionally substituted —CH₂—CH═CH—(CH₂)₂—C(O)—.
298. The agent of any one of Embodiments 181-290, wherein a staple is —CH₂—CH═CH—(CH₂)₂—C(O)—.
299. The agent of Embodiment 295-298, wherein —CH═CH— is E.
300. The agent of Embodiment 295-298, wherein —CH═CH— is Z.
301. The agent of any one of Embodiments 181-290, wherein a staple is optionally substituted —(CH₂)ₙ—, wherein n is 1-20.
302. The agent of any one of Embodiments 181-290, wherein a staple is —(CH₂)ₙ—, wherein n is 1-20.
303. The agent of any one of Embodiments 181-290, wherein a staple is optionally substituted —(CH₂)ₙ—CO—, wherein n is 1-20.
304. The agent of any one of Embodiments 181-290, wherein a staple is —(CH₂)ₙ—C(O)—, wherein n is 1-20.
305. The agent of Embodiment 301-304, wherein n is 4-10.
306. The agent of Embodiment 301-304, wherein n is 5-8.
307. The agent of Embodiment 301-304, wherein n is 6.
308. The agent of any one of Embodiments 291-307, wherein optionally substituted —(CH₂)₃— or —C(O)— is bonded to an amino acid residue closer to a N-terminus to the other amino acid residue bonded to the same staple.
309. The agent of any one of Embodiments 291-308, wherein optionally substituted —(CH₂)₃— or —C(O)— is bonded to an alpha-carbon atom of an amino acid residue.
310. The agent of any one of Embodiments 291-308, wherein optionally substituted —(CH₂)₃— or —C(O)— is bonded to a nitrogen atom of an amino acid residue.
311. The agent of any one of Embodiments 291-308, wherein optionally substituted —(CH₂)₃— or —C(O)— is bonded to a nitrogen atom bonded to an alpha carbon atom of an amino acid residue.
312. The agent of any one of Embodiments 291-311, wherein optionally substituted —(CH₂)₃— or —C(O)— is bonded to X¹.
313. The agent of Embodiment 312, wherein the other amino acid residue bonded to the staple is X⁴.
314. The agent of any one of Embodiments 181-313, wherein a staple is —(CH₂)m-N(R′)—(CH₂)ₙ—CH═CH—(CH₂)n′-, wherein each of m, n and n′ is independently 1, 2, 3, 4, 5, 6, 7, 8, 9, or 10, and each —CH₂— is independently optionally substituted.
315. The agent of any one of Embodiments 181-313, wherein a staple is —(CH₂)m-N(R′)—(CH₂)ₙ—CH═CH—(CH₂)n′-, wherein each of m, n and n′ is independently 1, 2, 3, 4, 5, 6, 7, 8, 9, or 10.
316. The agent of any one of Embodiments 181-315, wherein a staple is —(CH₂)m-N(R′)—C(O)—O—(CH₂)ₙ—CH═CH—(CH₂)n′-, wherein each of m, n and n′ is independently 1, 2, 3, 4, 5, 6, 7, 8, 9, or 10, and each —CH₂— is independently optionally substituted.
317. The agent of any one of Embodiments 181-315, wherein a staple is —(CH₂)m-N(R′)—C(O)—O—(CH₂)ₙ—CH═CH—(CH₂)n′-, wherein each of m, n and n′ is independently 1, 2, 3, 4, 5, 6, 7, 8, 9, or 10.
318. The agent of any one of Embodiments 314-317, wherein the —CH═CH— is E.
319. The agent of any one of Embodiments 314-317, wherein the —CH═CH— is Z.
320. The agent of any one of Embodiments 181-319, wherein a staple is —(CH₂)m-N(R′)—(CH₂)ₙ—CH₂—CH₂—(CH₂)n′-, wherein each of m, n and n′ is independently 1, 2, 3, 4, 5, 6, 7, 8, 9, or 10, and each —CH₂— is independently optionally substituted.
321. The agent of any one of Embodiments 181-320, wherein a staple is —(CH₂)m-N(R′)—(CH₂)ₙ—CH₂—CH₂—(CH₂)n′-, wherein each of m, n and n′ is independently 1, 2, 3, 4, 5, 6, 7, 8, 9, or 10.
322. The agent of any one of Embodiments 181-321, wherein a staple is —(CH₂)m-N(R′)—C(O)—O—(CH₂)ₙ—CH₂—CH₂—(CH₂)n′-, wherein each of m, n and n′ is independently 1, 2, 3, 4, 5, 6, 7, 8, 9, or 10, and each —CH₂— is independently optionally substituted.
323. The agent of any one of Embodiments 181-322, wherein a staple is —(CH₂)m-N(R′)—C(O)—O—(CH₂)ₙ—CH₂—CH₂—(CH₂)n′-, wherein each of m, n and n′ is independently 1, 2, 3, 4, 5, 6, 7, 8, 9, or 10.
324. The agent of any one of Embodiments 314-323, wherein —(CH₂)m- is bonded an amino acid residue closer to a N-terminus to the other amino acid residue bonded to the same staple.
325. The agent of any one of Embodiments 314-323, wherein —(CH₂)m- is bonded an amino acid residue closer to a C-terminus to the other amino acid residue bonded to the same staple.
326. The agent of any one of Embodiments 314-325, wherein m is 1.
327. The agent of any one of Embodiments 314-325, wherein m is 2.
328. The agent of any one of Embodiments 314-327, wherein n is 1.
329. The agent of any one of Embodiments 314-327, wherein n is 2.
330. The agent of any one of Embodiments 314-327, wherein n is 3.
331. The agent of any one of Embodiments 314-330, wherein n′ is 3.
332. The agent of any one of Embodiments 314-331, wherein R′ is —H.
333. The agent of any one of Embodiments 314-331, wherein R′ is optionally substituted C₁₋₆ aliphatic.
334. The agent of any one of Embodiments 314-331, wherein R′ is methyl.
335. The agent of any one of Embodiments 314-331, wherein R′ is taken together with R^{a3} of the amino acid residue to which L^{s3} is bonded to and their intervening atom(s) to form an optionally substituted 3-10 membered ring having 0-5 heteroatoms in addition to the intervening atom(s).
336. The agent of any one of Embodiments 314-331, wherein R′ is taken together with R^{a3} of the amino acid residue to which L^{s3} is bonded to and their intervening atom(s) to form a 3-10 membered monocyclic ring having 0-5 heteroatoms in addition to the intervening atom(s).
337. The agent of any one of Embodiments 335-336, wherein the formed ring is saturated.
338. The agent of any one of Embodiments 335-337, wherein the formed ring is 4-membered.
339. The agent of any one of Embodiments 335-338, wherein the formed ring is 5-membered.
340. The agent of any one of Embodiments 335-339, wherein the formed ring has no heteroatoms in addition to the intervening atom(s).
341. The agent of any one of Embodiments 181-340, wherein a staple is optionally substituted —*CH₂—N(—CH₂—**CH₂—)—C(O)O—(CH₂)₃—CH═CH—(CH₂)₃—, wherein —*CH₂— and —**CH₂— are bonded to the same amino acid residue.
342. The agent of any one of Embodiments 181-340, wherein a staple is —*CH₂—N(—CH₂—**CH₂—)—C(O)O—(CH₂)₃—CH═CH—(CH₂)₃—, wherein —*CH₂— and —**CH₂— are bonded to the same amino acid residue.
343. The agent of Embodiment 341-342, wherein —CH═CH— is E.
344. The agent of Embodiment 341-342, wherein —CH═CH— is Z.
345. The agent of any one of Embodiments 181-340, wherein a staple is optionally substituted —*CH₂—N(—CH₂—**CH₂—)—C(O)O—(CH₂)₃—CH₂—CH₂—(CH₂)₃—, wherein —*CH₂— and —**CH₂— are bonded to the same amino acid residue.
346. The agent of any one of Embodiments 181-340, wherein a staple is —*CH₂—N(—CH₂—**CH₂—)—C(O)O—(CH₂)₃—CH₂—CH₂—(CH₂)₃—, wherein —*CH₂— and —**CH₂— are bonded to the same amino acid residue.
347. The agent of any one of Embodiments 341-346, wherein —*CH₂— and —**CH₂— are bonded to the same atom.
348. The agent of any one of Embodiments 314-347, wherein optionally substituted —(CH₂)m or —*CH₂— is bonded to an amino acid residue closer to a C-terminus to the other amino acid residue bonded to the same staple.
349. The agent of any one of Embodiments 314-348, wherein optionally substituted —(CH₂)m or —*CH₂— is bonded to an alpha-carbon atom of an amino acid residue.
350. The agent of any one of Embodiments 314-349, wherein optionally substituted —(CH₂)m or —*CH₂— is bonded to X¹¹.
351. The agent of Embodiment 350, wherein the other amino acid residue bonded to the staple is X⁴. 352. The agent of any one of Embodiments 181-313, wherein a staple is optionally substituted —(CH₂)m-CH═CH—(CH₂)ₙ—.
353. The agent of any one of Embodiments 181-313, wherein a staple is —(CH₂)m-CH═CH—(CH₂)ₙ—.
354. The agent of any one of Embodiments 181-313, wherein a staple is optionally substituted —(CH₂)m-CH₂—CH₂—(CH₂)ₙ—.
355. The agent of any one of Embodiments 181-313, wherein a staple is —(CH₂)m-CH₂—CH₂—(CH₂)ₙ—.
356. The agent of any one of Embodiments 352-355, wherein m is 1.
357. The agent of any one of Embodiments 352-355, wherein m is 2.
358. The agent of any one of Embodiments 352-355, wherein m is 3.
359. The agent of any one of Embodiments 352-355, wherein m is 4.
360. The agent of any one of Embodiments 352-355, wherein m is 5.
361. The agent of any one of Embodiments 352-355, wherein m is 6.
362. The agent of any one of Embodiments 352-355, wherein m is 7.
363. The agent of any one of Embodiments 352-355, wherein m is 8.
364. The agent of any one of Embodiments 352-363, wherein n is 1.
365. The agent of any one of Embodiments 352-363, wherein n is 2.
366. The agent of any one of Embodiments 352-363, wherein n is 3.
367. The agent of any one of Embodiments 352-363, wherein n is 4.
368. The agent of any one of Embodiments 352-363, wherein n is 5.
369. The agent of any one of Embodiments 352-363, wherein n is 6.
370. The agent of any one of Embodiments 352-363, wherein n is 7.
371. The agent of any one of Embodiments 352-363, wherein n is 8.
372. The agent of any one of Embodiments 352-371, wherein the staple is boned to X⁴ and X¹¹.
373. The agent of any one of the preceding Embodiments, wherein a staple has a length of 5-10 chain atoms.
374. The agent of Embodiment 373, wherein the length is 5 chain atoms.
375. The agent of Embodiment 373, wherein the length is 6 chain atoms.
376. The agent of Embodiment 373, wherein the length is 7 chain atoms.
377. The agent of any one of Embodiments 373-376, wherein the staple is a (i, i+3) staple.
378. The agent of any one of the preceding Embodiments, wherein a staple has a length of 10-25 chain atoms.
379. The agent of Embodiment 378, wherein the length is 12 chain atoms.
380. The agent of Embodiment 378, wherein the length is 13 chain atoms.
381. The agent of Embodiment 378, wherein the length is 14 chain atoms.
382. The agent of any one of Embodiments 378-381, wherein the staple is a (i, i+7) staple.
383. The agent of any one of the any one of the preceding Embodiments, wherein X¹ is a residue of an amino acid having the structure of formula A-I, A-II or A-III, wherein R^{a2} and R^{a3} are taken together with their intervening atom(s) to form an optionally substituted 3-10 membered ring having 0-5 heteroatoms in addition to the intervening atom(s).
384. The agent of any one of the preceding Embodiments, wherein X¹ is —N(R^{a1})-L^{a1}-C(-L^{a}-R^{SP})(R^{a3})-L^{a2}-C(O)—.
385. The agent of Embodiment 384, wherein R^{a1} is —H.
386. The agent of any one of Embodiments 384-385, wherein R^{a3} is —H.
387. The agent of any one of Embodiments 384-385, wherein R^{a3} is optionally substituted C₁₋₆ aliphatic.
388. The agent of Embodiment 384, wherein R^{a1} and R^{a3} are taken together with their intervening atom(s) to form an optionally substituted 3-10 membered ring having 0-3 heteroatoms in addition to the intervening atom(s).
389. The agent of Embodiment 388, wherein R^{a1} and R^{a3} are taken together with their intervening atom(s) to form an 5-membered saturated ring having no heteroatoms in addition to the nitrogen to which R^{a1} is attached.
390. The agent of any one of Embodiments 384-389, wherein L^{a1} is a covalent bond.
391. The agent of any one of Embodiments 384-390, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
392. The agent of any one of Embodiments 384-390, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
393. The agent of any one of Embodiments 384-390, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
394. The agent of any one of Embodiments 384-391, wherein L^{a} is optionally substituted —(CH₂)ₙ— wherein n is 1, 2, 3, 4, 5, or 6.
395. The agent of any one of Embodiments 384-394, wherein L^{a} is —(CH₂)ₙ— wherein n is 1, 2, 3, 4, 5, or 6.
396. The agent of any one of Embodiments 384-395, wherein L^{a2} is a covalent bond.
397. The agent of any one of Embodiments 384-396, wherein R^{SP1} is optionally substituted —CH═CH₂.
398. The agent of any one of Embodiments 384-396, wherein R^{SP1} is —CH═CH₂.
399. The agent of any one of the preceding Embodiments, wherein X⁴ is a residue of an amino acid that comprises two olefins each independently suitable for stapling.
400. The agent of any one of the preceding Embodiments, wherein X⁴ is —N(R^{a1} L^{a1}-C(-L^{a}-R^{SP1})(R^{a3})-L^{a2}-C(O)—
401. The agent of Embodiment 400, wherein R^{a1} is —H.
402. The agent of any one of Embodiments 400-401, wherein R^{a3} is —H.
403. The agent of any one of Embodiments 400-401, wherein R^{a3} is optionally substituted C₁−6 aliphatic.
404. The agent of any one of the preceding Embodiments, wherein X⁴ is —N(R^{a1})-L^{a1}-C(-L^{a}-R^{SP1})(-L^{a}-R^{SP2})-L^{a2}-C(O)—.
405. The agent of any one of Embodiments 400-404, wherein L^{a1} is a covalent bond.
406. The agent of any one of Embodiments 400-405, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
407. The agent of any one of Embodiments 400-405, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
408. The agent of any one of Embodiments 400-405, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
409. The agent of any one of Embodiments 400-406, wherein L^{a} bonded to R^{SP1} is optionally substituted —(CH₂)ₙ— wherein n is 1, 2, 3, 4, 5, or 6.
410. The agent of any one of Embodiments 400-406, wherein L^{a} bonded to R^{SP1} is —(CH₂)ₙ— wherein n is 1, 2, 3, 4, 5, or 6.
411. The agent of any one of Embodiments 400-410, wherein L^{a2} is a covalent bond.
412. The agent of any one of Embodiments 400-411, wherein R^{SP1} is optionally substituted —CH═CH₂.
413. The agent of any one of Embodiments 400-411, wherein R^{SP1} is —CH═CH₂.
414. The agent of any one of Embodiments 400-413, wherein R^{SP2} is optionally substituted —CH═CH₂.
415. The agent of any one of Embodiments 400-413, wherein R^{SP2} is —CH═CH₂.
416. The agent of any one of Embodiments 400-415, wherein L^{a} bonded to R^{SP1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
417. The agent of any one of Embodiments 400-415, wherein L^{a} bonded to R^{SP1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
418. The agent of any one of Embodiments 400-415, wherein L^{a} bonded to R^{SP1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
419. The agent of any one of Embodiments 400-415, wherein L^{a} bonded to R^{SP1} is optionally substituted —(CH₂)ₙ— wherein n is 1, 2, 3, 4, 5, or 6.
420. The agent of any one of Embodiments 400-415, wherein L^{a} bonded to R^{SP1} is —(CH₂)ₙ— wherein n is 1, 2, 3, 4, 5, or 6.
421. The agent of any one of Embodiments 404-420, wherein L^{a} bonded to R^{SP2} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
422. The agent of any one of Embodiments 404-420, wherein L^{a} bonded to R^{SP2} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
423. The agent of any one of Embodiments 404-420, wherein L^{a} bonded to R^{SP2} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
424. The agent of any one of Embodiments 404-420, wherein L^{a} bonded to R^{SP2} is optionally substituted —(CH₂)ₙ— wherein n is 1, 2, 3, 4, 5, or 6.
425. The agent of any one of Embodiments 404-420, wherein L^{a} bonded to R^{SP2} is —(CH₂)ₙ— wherein n is 1, 2, 3, 4, 5, or 6.
426. The agent of any one of the preceding Embodiments, wherein X⁴ is a residue of an amino acid having the structure of formula A-I, A-II or A-III, wherein R^{a2} and R^{a3} independently comprises an olefin.
427. The agent of any one of the preceding Embodiments, wherein X⁴ is a residue of an amino acid having the structure of formula A-I, A-II or A-III, wherein R^{a2} and R^{a3} are independently -L^{a}-CH═CH₂.
428. The agent of any one of the preceding Embodiments, wherein X⁴ is B5.
429. The agent of any one of the preceding Embodiments, wherein X¹¹ is an amino acid residue suitable for stapling.
430. The agent of any one of the preceding Embodiments, wherein X¹¹ is —N(R^{a1})-L^{a1}-C(-L^{a}-R^{SP1})(R^{a3})-L^{a2}-C(O)—.
431. The agent of Embodiment 430, wherein R^{a1} is —H.
432. The agent of any one of Embodiments 430-431, wherein L^{a1} is a covalent bond.
433. The agent of any one of Embodiments 430-432, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
434. The agent of any one of Embodiments 430-432, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
435. The agent of any one of Embodiments 430-432, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
436. The agent of any one of Embodiments 430-433, wherein L^{a} is —(CH₂)ₙ— wherein n is 1, 2, 3, 4, 5, or 6.
437. The agent of any one of Embodiments 430-436, wherein L^{a2} is a covalent bond.
438. The agent of any one of Embodiments 430-437, wherein R^{SP1} is optionally substituted —CH═CH₂.
439. The agent of any one of Embodiments 430-437, wherein R^{SP1} is —CH═CH₂.
440. The agent of any one of Embodiments 430-439, wherein one methylene unit of L is replaced with —N(R′)—.
441. The agent of any one of Embodiments 430-439, wherein one methylene unit of L is replaced with —N(R′)C(O)O—.
442. The agent of any one of Embodiments 440-441, wherein R′ is —H.
443. The agent of any one of Embodiments 440-441, wherein R′ is C₁₋₆ aliphatic.
444. The agent of any one of Embodiments 440-441, wherein R′ and R^{a3} are taken together with their intervening atom(s) to form an optionally substituted 3-14 membered ring having 0-5 heteroatoms in addition to the nitrogen atom to which R′ is attached.
445. The agent of any one of Embodiments 440-441, wherein R′ and R^{a3} are taken together with their intervening atom(s) to form an optionally substituted 3-8 membered ring having 0-5 heteroatoms in addition to the nitrogen atom to which R′ is attached.
446. The agent of any one of Embodiments 440-441, wherein R′ and R^{a3} are taken together with their intervening atom(s) to form an optionally substituted 3-7 membered ring having no heteroatoms in addition to the nitrogen atom to which R′ is attached.
447. The agent of any one of Embodiments 444-446, wherein the ring is monocyclic.
448. The agent of any one of Embodiments 444-447, wherein the ring is saturated.
449. The agent of any one of Embodiments 444-448, wherein the ring is 5-membered.
450. The agent of any one of Embodiments 430-443, wherein R^{a3} is —H.
451. The agent of any one of Embodiments 430-443, wherein R^{a3} is optionally substituted C₁₋₆ aliphatic.
452. The agent of any one of Embodiments 430-439 and 450-451, wherein L^{a} is optionally substituted —(CH₂)ₙ— wherein n is 1, 2, 3, 4, 5, or 6.
453. The agent of Embodiment 452, wherein L^{a} is —(CH₂)ₙ— wherein n is 1, 2, 3, 4, 5, or 6.
454. The agent of any one of the preceding Embodiments, wherein X¹¹ is PyrS2, S8, PyrS, S7, PyrS3, SeN, Az, S4, S6, SdN, S10, S5, SgN or PyrS1.
455. The agent of Embodiment 450, wherein X¹¹ is PyrS2.
456. The agent of Embodiment 450, wherein X¹¹ is S8.
457. The agent of Embodiment 450, wherein X¹¹ is PyrS3.
458. The agent of any one of the preceding Embodiments, wherein X¹ is selected from PL3, Phe, Ala, Aib, Pro, alphaMePro or Asp.
459. The agent of any one of the preceding Embodiments, wherein X¹ is selected from Bn3OAllyl, BzAm3Oallyl, HypBzEs3OAllyl, HypEs4, HypEs5, HypPAc3OAllyl, MePro, NMebAla, PAc3OAllyl, ProAm5, ProAm6, ProBzAm3OAllyl, ProPAc3OAllyl, ProSAm3, PyrR, Sar, Aib, Ala, Asp, Gly, Phe, PL3, Pro, R3, and R5.
460. The agent of any one of the preceding Embodiments, wherein X¹ is selected from Bn3OAllyl, BzAm3Oallyl, HypBzEs3OAllyl, HypEs4, HypEs5, HypPAc3OAllyl, MePro, NMebAla, PAc3OAllyl, ProAm5, ProAm6, ProBzAm3OAllyl, ProPAc3OAllyl, ProSAm3, PyrR, and Sar.
461. The agent of any one of the preceding Embodiments, wherein X¹ is selected from Aib, Ala, Asp, Gly, Phe, PL3, Pro, R3, and R5.
462. The agent of any one of the preceding Embodiments, wherein X² is selected from Asp, RbGlu, Phe, Asn, Ile, Glu, NMeD, His, Ala, tetz, isoDAsp, Ser, Gln, Leu, Hse, Dab, Tyr, 3MeF, 3FF, [MeSO2]Dap, [Tf]Dap and SbGlu.
463. The agent of any one of the preceding Embodiments, wherein X² is selected from Asp, Hse, Asn, Glu, RbGlu, SbGlu, and isoDAsp.
464. The agent of any one of the preceding Embodiments, wherein X² is selected from [CH2CMe2CO2H]TriAzDap, [CMe2CO2H]TriAzDap, [Et]AspE, [EtSSEt]AspE, [EtSSHex]AspE, [EtSSPh]AspE, [EtSSpy]AspE, [Me]AspE, AcAsp, AspSH, RbOHAsp, [MeSO2]Dap, [Tf]Dap,
2COOHF, 3COOHF, 3FF, 3MeF, 4Thz, 4Tria, Aad, Ala, Asn, Asp, Dab, Gln, Glu, His, Hse, Ile, isoDAsp, Leu, NMeD, Phe, PL3, R3, R5, RbGlu, SbGlu, Ser, tetz, TfeGA, Thr, and Tyr.
465. The agent of any one of the preceding Embodiments, wherein X² is selected from [CH₂CMe2CO2H]TriAzDap, [CMe2CO2H]TriAzDap, [Et]AspE, [EtSSEt]AspE, [EtSSHex]AspE, [EtSSPh]AspE, [EtSSpy]AspE, [Me]AspE, AcAsp, AspSH, and RbOHAsp.
466. The agent of any one of the preceding Embodiments, wherein X² is selected from [MeSO2]Dap, [Tf]Dap, 2COOHF, 3COOHF, 3FF, 3MeF, 4Thz, 4Tria, Aad, Ala, Asn, Asp, Dab, Gln, Glu, His, Hse, Ile, isoDAsp, Leu, NMeD, Phe, PL3, R3, R5, RbGlu, SbGlu, Ser, tetz, TfeGA, Thr, and Tyr.
467. The agent of any one of the preceding Embodiments, wherein X² comprises a side chain comprising an acid group.
468. The agent of any one of the preceding Embodiments, wherein X² comprises a side chain comprising —COOH or a salt form thereof.
469. The agent of any one of the preceding Embodiments, wherein X² is —N(R^{a1})-L^{a1}-C(R^{a2})(R^{a3})-L^{a2}-C(O)—.
470. The agent of Embodiment 469, wherein R^{a1} is —H.
471. The agent of any one of Embodiments 469-470, wherein R^{a3} is —H.
472. The agent of any one of Embodiments 469-470, wherein R^{a3} is optionally substituted C₁₋₆ aliphatic.
473. The agent of any one of Embodiments 469-472, wherein L^{a1} is a covalent bond
474. The agent of any one of Embodiments 469-473, wherein L^{a2} is a covalent bond.
475. The agent of any one of Embodiments 469-474, wherein R^{a2} is or comprises an acidic or polar group.
476. The agent of any one of Embodiments 469-475, wherein R^{a2} is -L″—COOH.
477. The agent of any one of Embodiments 469-475, wherein R^{a2} is -L″-Cy-COOH.
478. The agent of Embodiment 477, wherein -Cy- is optionally substituted phenylene.
479. The agent of any one of Embodiments 469-475, wherein R^{a2} is -L″—C(O)N(R′)₂.
480. The agent of any one of Embodiments 476-479, wherein L″ is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
481. The agent of any one of Embodiments 476-479, wherein L″ is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
482. The agent of any one of Embodiments 476-479, wherein L″ is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
483. The agent of any one of Embodiments 476-480, wherein L″ is optionally substituted —(CH₂)ₙ— wherein n is 1, 2, 3, 4, 5, or 6.
484. The agent of any one of Embodiments 476-483, wherein L″ is —(CH₂)ₙ— wherein n is 1, 2, 3, 4, 5, or 6.
485. The agent of any one of the preceding Embodiments, wherein X² is Asp.
486. The agent of any one of Embodiments 1-462, wherein X² comprises a side chain comprising a polar group.
487. The agent of any one of Embodiments 1-462, wherein X² comprises a side chain comprising —OH.
488. The agent of any one of Embodiments 1-462, wherein X² comprises a side chain comprising an amide group.
489. The agent of any one of the preceding Embodiments, wherein X³ is —N(R^{a1})-L^{a1}-C(-L^{a}-R^{SP1})(R^{a3})-L^{a2}-C(O)—.
490. The agent of Embodiment 489, wherein R^{a1} is —H.
491. The agent of any one of Embodiments 489-490, wherein R^{a3} is —H.
492. The agent of any one of Embodiments 489-490, wherein R^{a3} is optionally substituted C₁−6 aliphatic.
493. The agent of any one of Embodiments 489-492, wherein L^{a1} is a covalent bond.
494. The agent of any one of Embodiments 489-493, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
495. The agent of any one of Embodiments 489-493, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
496. The agent of any one of Embodiments 489-493, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
497. The agent of any one of Embodiments 489-494, wherein L^{a} is optionally substituted —(CH₂)ₙ— wherein n is 1, 2, 3, 4, 5, or 6.
498. The agent of any one of Embodiments 489-494, wherein L^{a} is —(CH₂)ₙ— wherein n is 1, 2, 3, 4, 5, or 6.
499. The agent of any one of Embodiments 489-498, wherein L^{a2} is a covalent bond.
500. The agent of any one of Embodiments 489-499, wherein R^{SP1} is optionally substituted —CH═CH₂.
501. The agent of any one of Embodiments 489-499, wherein R^{SP1} is —CH═CH₂.
502. The agent of any one of the preceding Embodiments, wherein X³ is selected from Npg, Ala, Ile, Leu, Cha, Phe, Abu, hLeu, RdN, 1NapA, 2NapA, R8, Val, F3CA, [AzAc]Lys, Gln, aIle, Nva, TOMe, hSe, S(Ome), nLeu, Thr, Asn, Ser, and HF2CA.
503. The agent of any one of the preceding Embodiments, wherein X³ comprises a hydrophobic side chain.
504. The agent of any one of the preceding Embodiments, wherein X³ is —N(R^{a1})-L^{a1}-C(R^{a2})(R^{a3})-L^{a2}-C(O)—.
505. The agent of Embodiment 504, wherein X³ is —N(R^{a1})—C(R^{a2})(R^{a3})—C(O)—.
506. The agent of Embodiment 504, wherein X³ is —NH—C(R^{a2})(R^{a3})—C(O)—.
507. The agent of any one of Embodiments 504-506, wherein R^{a2} and R^{a3} are independently hydrogen or optionally substituted C₁₋₁₀ aliphatic.
508. The agent of any one of Embodiments 504-506, wherein one of R^{a2} and R^{a3} is hydrogen and the other is C₁₋₁₀ aliphatic.
509. The agent of any one of Embodiments 504-506, wherein R^{a2} and R^{a3} are taken together with the carbon atom to which they are attached to form an optionally substituted 3-8 membered ring having 1-3 heteroatoms.
510. The agent of any one of Embodiments 504-506, wherein R^{a2} and R^{a3} are taken together with the carbon atom to which they are attached to form 3-8 membered cycloalkyl.
511. The agent of any one of the preceding Embodiments, wherein the side chain of X³ is C₁₋₁₀ alkyl optionally substituted with one or more substituents independently selected from halogen, —SR and —OR, wherein each R is independently C₁₋₄ alkyl.
512. The agent of any one of the preceding Embodiments, wherein the side chain of X³ is C₁₋₁₀ alkyl.
513. The agent of any one of the preceding Embodiments, wherein X³ is not stapled.
514. The agent of any one of the preceding Embodiments, wherein X³ is Npg.
515. The agent of any one of Embodiments 1-502, wherein X³ is an amino acid residue suitable for stapling.
516. The agent of any one of Embodiments 1-502, wherein X³ is stapled with X¹⁰.
517. The agent of Embodiment 516, wherein the X³—X¹⁰ staple is the only staple in the peptide.
518. The agent of any one of Embodiments 515-517, wherein X³ is RdN or R8.
519. The agent of any one of the preceding Embodiments, wherein X³ is selected from [lithocholate]-Lys, [lithocholate-PEG2]-Lys, [Me3AdamantC]-Lys, [Me3AdamantC-PEG2]-Lys, Npa, 1NapA, 2NapA, Abu, aIle, Ala, Asn, Asp, B5, Cha, F3CA, Gln, Glu, HF2CA, hLeu, hSe, Ile, iPrLys, Leu, Lys, nLeu, Npg, Nva, Phe, R8, RdN, S(Ome), Ser, TfeGA, Thr, TOMe, Trp, Tyr, and Val.
520. The agent of any one of the preceding Embodiments, wherein X³ is selected from [lithocholate]-Lys, [lithocholate-PEG2]-Lys, [Me3AdamantC]-Lys, [Me3AdamantC-PEG2]-Lys, and Npa.
521. The agent of any one of the preceding Embodiments, wherein X³ is selected from 1NapA, 2NapA, Abu, aIle, Ala, Asn, Asp, B5, Cha, F3CA, Gln, Glu, HF2CA, hLeu, hSe, Ile, iPrLys, Leu, Lys, nLeu, Npg, Nva, Phe, R8, RdN, S(Ome), Ser, TfeGA, Thr, TOMe, Trp, Tyr, and Val.
522. The agent of any one of the preceding Embodiments, wherein X⁴ is selected from B5, R8, RdN, R5, Ala, RgN, ReN, R7, Az, Asp, R6, and R4.
523. The agent of any one of the preceding Embodiments, wherein X⁴ and X¹¹ are stapled.
524. The agent of Embodiment 523, wherein the X⁴—X¹ staple is the only staple in the peptide.
525. The agent of Embodiment 523 or 524, wherein X⁴ is selected from R8, RdN, R5, RgN, ReN, R7, Az, R6, and R4.
526. The agent of any one of the preceding Embodiments, wherein X⁴ is selected from B3, B4, B6, Aib, Ala, Asp, Az, B5, Npg, R3, R4, R5, R6, R7, R8, RdN, ReN, RgN, S3, S4, S5, and S6.
527. The agent of any one of the preceding Embodiments, wherein X⁴ is selected from B3, B4, and B6.
528. The agent of any one of the preceding Embodiments, wherein X⁴ is selected from Aib, Ala, Asp, Az, B5, Npg, R3, R4, R5, R6, R7, R8, RdN, ReN, RgN, S3, S4, S5, and S6.
529. The agent of any one of Embodiments 1-522, wherein X⁴ is not stapled.
530. The agent of Embodiment 529, wherein X⁴ is Ala or Asp.
531. The agent of any one of the preceding Embodiments, wherein X⁵ is selected from Asp, Hse, Asn, Glu, tetz, 3Thi, hPhe, 2pyrA, Ala, [MeSO2]Dap, [Tf]Dap, Ser, Gln, Leu, Dab, [MeSO2]Dab, nLeu, His,
3pyrA, 4pyrA, [NHiPr]AsnR, [NHEt]AsnR, [NHnPr]AsnR, [NHCyPr]AsnR, [NHCyBu]AsnR, [NHMe]AsnR, Phe, isoAsp, isoDAsp, RbGlu, and SbGlu.
532. The agent of any one of the preceding Embodiments, wherein X⁵ is selected from [Et]AspE, [EtSSEt]AspE, [EtSSHex]AspE, [EtSSPh]AspE, [EtSSpy]AspE, [Me]AspE, AspSH, bMe2Asp, [MeSO2]Dab, [MeSO2]Dap, [NHCyBu]AsnR, [NHCyPr]AsnR, [NHEt]AsnR, [NHiPr]AsnR, [NHMe]AsnR, [NHnPr]AsnR, [Tf]Dap, 2pyrA, 3pyrA, 3Thi, 4pyrA, Ala, Arg, Asn, Asp, B5, BztA, Dab, Gln, Glu, His, hPhe, Hse, isoAsp, isoDAsp, Leu, nLeu, Npg, RbGlu, SbGlu, Ser, tetz, and Thr.
533. The agent of any one of the preceding Embodiments, wherein X⁵ is selected from [Et]AspE, [EtSSEt]AspE, [EtSSHex]AspE, [EtSSPh]AspE, [EtSSpy]AspE, [Me]AspE, AspSH, and bMe2Asp.
534. The agent of any one of the preceding Embodiments, wherein X⁵ is selected from [MeSO2]Dab, [MeSO2]Dap, [NHCyBu]AsnR, [NHCyPr]AsnR, [NHEt]AsnR, [NHiPr]AsnR, [NHMe]AsnR, [NHnPr]AsnR, [Tf]Dap, 2pyrA, 3pyrA, 3Thi, 4pyrA, Ala, Arg, Asn, Asp, B5, BztA, Dab, Gln, Glu, His, hPhe, Hse, isoAsp, isoDAsp, Leu, nLeu, Npg, RbGlu, SbGlu, Ser, tetz, and Thr.
535. The agent of any one of the preceding Embodiments, wherein X⁵ is selected from Asp, Asn, Gln, Glu, Hse, and Ser.
536. The agent of any one of the preceding Embodiments, wherein X⁵ comprises a side chain comprising an acid group.
537. The agent of any one of the preceding Embodiments, wherein X⁵ is —N(R^{a1})-L^{a1}-C(R^{a2})(R^{a3})-L^{a2}-C(O)—.
538. The agent of Embodiment 537, wherein R^{a1} is —H.
539. The agent of any one of Embodiments 537-538, wherein R^{a3} is —H.
540. The agent of any one of Embodiments 537-538, wherein R^{a3} is optionally substituted C₁₋₆ aliphatic.
541. The agent of any one of Embodiments 537-540, wherein L^{a1} is a covalent bond.
542. The agent of any one of Embodiments 537-541, wherein L^{a2} is a covalent bond.
543. The agent of any one of Embodiments 537-542, wherein R^{a2} is or comprises an acidic or polar group.
544. The agent of any one of Embodiments 537-543, wherein R^{a2} is -L″—COOH.
545. The agent of any one of Embodiments 537-543, wherein R^{a2} is -L″-Cy-COOH.
546. The agent of Embodiment 545, wherein -Cy- is optionally substituted phenylene.
547. The agent of any one of Embodiments 537-543, wherein R^{a2} is -L″—C(O)N(R′)₂.
548. The agent of any one of Embodiments 544-547, wherein L″ is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
549. The agent of any one of Embodiments 544-547, wherein L″ is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
550. The agent of any one of Embodiments 544-547, wherein L″ is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
551. The agent of any one of Embodiments 544-548, wherein L″ is optionally substituted —(CH₂)ₙ— wherein n is 1, 2, 3, 4, 5, or 6.
552. The agent of any one of Embodiments 544-550, wherein L″ is —(CH₂)ₙ— wherein n is 1, 2, 3, 4, 5, or 6.
553. The agent of any one of the preceding Embodiments, wherein X⁵ comprises a side chain comprising —COOH or a salt form thereof.
554. The agent of any one of the preceding Embodiments, wherein X⁵ is Asp.
555. The agent of any one of Embodiments 1-531, wherein X⁵ comprises a side chain comprising a polar group.
556. The agent of any one of Embodiments 1-531, wherein X⁵ comprises a side chain comprising —OH.
557. The agent of any one of Embodiments 1-531, wherein X⁵ comprises a side chain comprising an amide group.
558. The agent of any one of the preceding Embodiments, wherein X⁶ is selected from Asp, Glu, TfeGA, Thr, EtGA, Asn, 3COOHF, HIs, Gln, 2NapA, 4COOHF, nLeu, Leu, Cit, Aad, Cha, hLeu, hPhe, Ala, 3PyrA, Bip, Tyr, aMeDF, Phe, 1NapA, DaMeL, 3F3MeF, 4F3MeF, tetz, Arg, 2COOHF, DGlu, BztA, Trp, 6F1NapA, 3FF, 4FF, 34FF, 2PyrA, 4PyrA, hTyr, Qui, DipA, 4AmPhe, 2Thi, 1meH, [iPr]GA, [Pfbn]GA, [Tfb]GA, [Bn]GA, Lys, [Tfp]Dap, 1AcAW, Ser, Val, and [MeSO2]Dap.
559. The agent of any one of the preceding Embodiments, wherein X⁶ is selected from [2COOH4NH2Ph]Dap, [2COOH4NO2Ph]Cys, [2COOH4NO2Ph]Dap, [2COOHPh]TriAzDab, [2COOHPh]TriAzDap, [2Nic]Dap, [3COOH4NO2Ph]Dap, [4AcMePip]GA, [4AcMePip]GAbu, [4CF3PhAc]GA, [4CF3PhAc]GAbu, [Ac]GA, [Ac]GAbu, [Bn]GAbu, [CCpCO2H]TriAzDap, [CF3CO]GA, [CH₂CChCO2H]TriAzDap, [CH₂CCpCO2H]TriAzDap, [CH₂CH₂CO2H]TriAzDab, [CH₂CH₂CO2H]TriAzDap, [CH₂CMe2CO2H]TriAzDab, [CH₂CMe2CO2H]TriAzDap, [CH₂CO2H]GAbu, [CH₂CO2H]TriAzDab, [CH₂CO2H]TriAzDap, [CMe2CO2H]TriAzDab, [CMe2CO2H]TriAzDap, [Et]AspE, [Et]GA, [Et]GAbu, [Et]GluE, [EtSSEt]AspE, [EtSSEt]GluE, [EtSSHex]AspE, [EtSSPh]AspE, [EtSSPh]GluE, [EtSSpy]GluE, [Me]AspE, [Me]GA, [Me]GluE, [MeMorphBz]GA, [MeMorphBz]GAbu, [MePipAc]GA, [MePipAc]GAbu, [MorphAc]GA, [MorphAc]GAbu, [MorphEt]GAbu, [NdiMeButC]GA, [NdiMeButC]GAbu, [Pfb]GA, [Pfbn]GAbu, [PfBz]GA, [PfBz]GAbu, [PfPhAc]GA, [PfPhAc]GAbu, [Pic]GA, [Pic]GAbu, [sBu]GA, [Tfb]GAbu, [Tfp]GA, [Tfp]GAbu, 3TzF, 4F3COOHF, 4TzF, 5F3Me3COOHF, 5iPr3COOHF, AspSH, GAbu, GluSH, R2COOPipA, R3COOPipA, S2COOPipA, S3COOPipA, [Bn]GA, [iPr]GA, [Me2NPr]GA, [Me2NPr]GAbu, [MeSO2]Dap, [Pfbn]GA, [Tfb]GA, [Tfp]Dap, 1AcAW, 1meH, 1NapA, 2COOHF, 2NapA, 2PyrA, 2Thi, 34FF, 3cbmf, 3COOHF, 3F3MeF, 3FF, 3PyrA, 3thi, 4AmPhe, 4COOHF, 4F3MeF, 4FF, 4PyrA, 6F1NapA, Aad, Ala, aMeDF, Arg, Asn, Asp, B5, Bip, BztA, Cha, Cit, DaMeL, DGlu, DipA, EtGA, GA, Gln, Glu, His, hLeu, hPhe, Hse, hTyr, Leu, Lys, nLeu, Npg, Pff, Phe, Qui, Ser, tetz, TfeGA, Thr, Trp, Tyr, and Val.
560. The agent of any one of the preceding Embodiments, wherein X⁶ is selected from [2COOH4NH2Ph]Dap, [2COOH4NO2Ph]Cys, [2COOH4NO2Ph]Dap, [2COOHPh]TriAzDab, [2COOHPh]TriAzDap, [2Nic]Dap, [3COOH4NO2Ph]Dap, [4AcMePip]GA, [4AcMePip]GAbu, [4CF3PhAc]GA, [4CF3PhAc]GAbu, [Ac]GA, [Ac]GAbu, [Bn]GAbu, [CCpCO2H]TriAzDap, [CF3CO]GA, [CH₂CChCO2H]TriAzDap, [CH₂CCpCO2H]TriAzDap, [CH₂CH₂CO2H]TriAzDab, [CH₂CH₂CO2H]TriAzDap, [CH₂CMe2CO2H]TriAzDab, [CH₂CMe2CO2H]TriAzDap, [CH₂CO2H]GAbu, [CH₂CO2H]TriAzDab, [CH₂CO2H]TriAzDap, [CMe2CO2H]TriAzDab, [CMe2CO2H]TriAzDap, [Et]AspE, [Et]GA, [Et]GAbu, [Et]GluE, [EtSSEt]AspE, [EtSSEt]GluE, [EtSSHex]AspE, [EtSSPh]AspE, [EtSSPh]GluE, [EtSSpy]GluE, [Me]AspE, [Me]GA, [Me]GluE, [MeMorphBz]GA, [MeMorphBz]GAbu, [MePipAc]GA, [MePipAc]GAbu, [MorphAc]GA, [MorphAc]GAbu, [MorphEt]GAbu, [NdiMeButC]GA, [NdiMeButC]GAbu, [Pfb]GA, [Pfbn]GAbu, [PfBz]GA, [PfBz]GAbu, [PfPhAc]GA, [PfPhAc]GAbu, [Pic]GA, [Pic]GAbu, [sBu]GA, [Tfb]GAbu, [Tfp]GA, [Tfp]GAbu, 3TzF, 4F3COOHF, 4TzF, 5F3Me3COOHF, 5iPr3COOHF, AspSH, GAbu, GluSH, R2COOPipA, R3COOPipA, S2COOPipA, and S3COOPipA.
561. The agent of any one of the preceding Embodiments, wherein X⁶ is selected from [Bn]GA, [iPr]GA, [Me2NPr]GA, [Me2NPr]GAbu, [MeSO2]Dap, [Pfbn]GA, [Tfb]GA, [Tfp]Dap, 1AcAW, 1meH, 1NapA, 2COOHF, 2NapA, 2PyrA, 2Thi, 34FF, 3cbmf, 3COOHF, 3F3MeF, 3FF, 3PyrA, 3thi, 4AmPhe, 4COOHF, 4F3MeF, 4FF, 4PyrA, 6F1NapA, Aad, Ala, aMeDF, Arg, Asn, Asp, B5, Bip, BztA, Cha, Cit, DaMeL, DGlu, DipA, EtGA, GA, Gln, Glu, His, hLeu, hPhe, Hse, hTyr, Leu, Lys, nLeu, Npg, Pff, Phe, Qui, Ser, tetz, TfeGA, Thr, Trp, Tyr, and Val.
562. The agent of any one of the preceding Embodiments, wherein X⁶ comprises a side chain comprising an acid or a polar group.
563. The agent of any one of the preceding Embodiments, wherein X⁶ comprises a side chain comprising an acid group.
564. The agent of any one of the preceding Embodiments, wherein X⁶ comprises a side chain comprising —COOH or a salt form thereof.
565. The agent of any one of the preceding Embodiments, wherein X⁶ is —N(R^{a1})-L^{a1}-C(R^{a2})(R^{a3})-L^{a2}-C(O)—.
566. The agent of Embodiment 565, wherein R^{a1} is —H.
567. The agent of any one of Embodiments 565-566, wherein R^{a3} is —H.
568. The agent of any one of Embodiments 565-566, wherein R^{a3} is optionally substituted C₁₋₆ aliphatic.
569. The agent of any one of Embodiments 565-568, wherein L^{a1} is a covalent bond.
570. The agent of any one of Embodiments 565-569, wherein L^{a2} is a covalent bond.
571. The agent of any one of Embodiments 565-570, wherein R^{a2} is or comprises an acidic or polar group.
572. The agent of any one of Embodiments 565-571, wherein R^{a2} is -L″—COOH.
573. The agent of any one of Embodiments 565-571, wherein R^{a2} is -L″-Cy-COOH.
574. The agent of Embodiment 573, wherein -Cy- is optionally substituted phenylene.
575. The agent of any one of Embodiments 565-571, wherein R^{a2} is -L″—C(O)N(R′)₂.
576. The agent of any one of Embodiments 572-575, wherein L″ is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
577. The agent of any one of Embodiments 572-575, wherein L″ is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
578. The agent of any one of Embodiments 572-575, wherein L″ is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
579. The agent of any one of Embodiments 572-576, wherein L″ is optionally substituted —(CH₂)ₙ— wherein n is 1, 2, 3, 4, 5, or 6.
580. The agent of any one of Embodiments 572-579, wherein L″ is —(CH₂)ₙ— wherein n is 1, 2, 3, 4, 5, or 6.
581. The agent of any one of Embodiments 572-578, wherein a methylene unit is replaced with —N(R′)—.
582. The agent of Embodiment 581, wherein R′ is —H.
583. The agent of Embodiment 581, wherein R′ is optionally substituted C₁₋₆ alkyl.
584. The agent of any one of the preceding Embodiments, wherein X⁶ is TfeGA.
585. The agent of any one of the preceding Embodiments, wherein X⁶ is 3COOHF.
586. The agent of any one of the preceding Embodiments, wherein X⁶ is 2COOHF.
587. The agent of any one of the preceding Embodiments, wherein X⁶ is Asp.
588. The agent of any one of the preceding Embodiments, wherein X⁶ is Aad.
589. The agent of any one of Embodiments 1-558, wherein X⁶ comprises a side chain comprising a polar group.
590. The agent of any one of Embodiments 1-558, wherein X⁶ comprises a side chain comprising —OH.
591. The agent of any one of Embodiments 1-558, wherein X⁶ comprises a side chain comprising an amide group.
592. The agent of any one of the preceding Embodiments, wherein X⁷ is a hydrophobic amino acid residue.
593. The agent of any one of the preceding Embodiments, wherein X⁷ is —N(R^{a1})-L^{a1}-C(R^{a2})(R^{a3})-L^{a2}-C(O)—.
594. The agent of any one of the preceding Embodiments, wherein X⁷ is —N(R^{a1})—C(R^{a2})(R^{a3})—C(O)—.
595. The agent of any one of the preceding Embodiments, wherein X⁷ is —NH—C(R^{a2})(R^{a3})—C(O)—.
596. The agent of any one of Embodiments 593-595, wherein R^{a2} and R^{a3} are independently hydrogen or optionally substituted C₁₋₁₀ aliphatic.
597. The agent of any one of Embodiments 593-595, wherein one of R^{a2} and R^{a3} is hydrogen and the other is C₁₋₁₀ aliphatic.
598. The agent of any one of Embodiments 593-595, wherein R^{a2} and R^{a3} are taken together with the carbon atom to which they are attached to form an optionally substituted 3-8 membered ring having 1-3 heteroatoms.
599. The agent of any one of Embodiments 593-595, wherein R^{a2} and R^{a3} are taken together with the carbon atom to which they are attached to form 3-8 membered cycloalkyl.
600. The agent of any one of the preceding Embodiments, wherein X⁷ is selected from Ala, Leu, iPrLys, Phe, Ser, Aib, Gln, nLeu, Trp, Ile, and Lys, and a substituted or labeled lysine.
601. The agent of any one of the preceding Embodiments, wherein X⁷ is selected from Ala, Leu, iPrLys, [AzAc]Lys, Phe, Ser, [FAM6Ppg][p1 TB]Lys, Aib, Gln, nLeu, Trp, [FAM6Ppg][1TriAc]Lys, Ile, and Lys.
602. The agent of any one of the preceding Embodiments, wherein X⁷ is selected from Ala, Leu, iPrLys, Phe, Ser, Aib, Gln, nLeu, Trp, Ile, and Lys.
603. The agent of any one of the preceding Embodiments, wherein X⁷ is selected from [20xoPpz]GlnR, [3Py]4SF, [4Pippip]GlnR, [Ac]Lys, [AcPpz]GlnR, [bismethoxyethylamine]GlnR, [CF3CO]Lys, [CH₂NMe2]4SEF, [EtSO2Ppz]GlnR, [isoindoline]GlnR, [Me2diaminobutane]GlnR, [Me2Npr]Lys, [Me2NPrPip]GlnR, [MeSO2]Lys, [Morph]GlnR, [NHEt]GlnR, [NMe2]GlnR, [Phc]Lys, [TfePpz]GlnR, Cpg, CyLeu, F2PipNva, hhLeu, Me2Asn, Me2Gln, MeGln, MePpzAsn, Met2O, MorphAsn, MorphNva, [MorphAc]Lys, [mPEG4]Lys, 3COOHF, Aib, Ala, Asn, Asp, Gln, Gly, His, Hse, Ile, iPrLys, Leu, Lys, nLeu, Phe, R5, Ser, Thr, and Trp.
604. The agent of any one of the preceding Embodiments, wherein X⁷ is selected from [20xoPpz]GlnR, [3Py]4SF, [4Pippip]GlnR, [Ac]Lys, [AcPpz]GlnR, [bismethoxyethylamine]GlnR, [CF3CO]Lys, [CH₂NMe2]4SEF, [EtSO2Ppz]GlnR, [isoindoline]GlnR, [Me2diaminobutane]GlnR, [Me2Npr]Lys, [Me2NPrPip]GlnR, [MeSO2]Lys, [Morph]GlnR, [NHEt]GlnR, [NMe2]GlnR, [Phc]Lys, [TfePpz]GlnR, Cpg, CyLeu, F2PipNva, hhLeu, Me2Asn, Me2Gln, MeGln, MePpzAsn, Met20, MorphAsn, and MorphNva.
605. The agent of any one of the preceding Embodiments, wherein X⁷ is selected from [MorphAc]Lys, [mPEG4]Lys, 3COOHF, Aib, Ala, Asn, Asp, Gln, Gly, His, Hse, Ile, iPrLys, Leu, Lys, nLeu, Phe, R5, Ser, Thr, and Trp.
606. The agent of any one of the preceding Embodiments, wherein X⁷ is Ala.
607. The agent of any one of the preceding Embodiments, wherein X⁸ is a hydrophobic amino acid residue.
608. The agent of any one of the preceding Embodiments, wherein X⁸ is —N(R^{a1})-L^{a1}-C(R^{a2})(R^{a3})-L^{a2}-C(O)—.
609. The agent of any one of the preceding Embodiments, wherein X⁸ is —N(R^{a1})—C(R^{a2})(R^{a3})—C(O)—.
610. The agent of any one of the preceding Embodiments, wherein X⁸ is —NH—C(R^{a2})(R^{a3})—C(O)—.
611. The agent of any one of Embodiments 608−610, wherein R^{a2} and R^{a3} are independently hydrogen or optionally substituted C₁₋₁₀ aliphatic.
612. The agent of any one of Embodiments 608−610, wherein one of R^{a2} and R^{a3} is hydrogen and the other is C₁-10 aliphatic.
613. The agent of any one of Embodiments 608−610, wherein R^{a2} and R^{a3} are taken together with the carbon atom to which they are attached to form an optionally substituted 3-8 membered ring having 1-3 heteroatoms.
614. The agent of any one of Embodiments 608−610, wherein R^{a2} and R^{a3} are taken together with the carbon atom to which they are attached to form 3-8 membered cycloalkyl.
615. The agent of any one of the preceding Embodiments, wherein X⁸ is selected from The agent of any one of the preceding Embodiments, wherein X⁸ is selected from Ala, Leu, Phe, Ser, Aib, Asp, Glu, Aad, Trp, nLeu, Gln, Ile, Lys, iPrLys, and a substituted or labeled lysine.
616. The agent of any one of the preceding Embodiments, wherein X⁸ is selected from Ala, Leu, Phe, Ser, Aib, Asp, Glu, Aad, Trp, nLeu, [mPEG2]Lys, [AzAc]Lys, Gln, [FAM6Ppg][1TriAc]Lys, [35CF3PhPr]Lys, [1NapPr]Lys, [22PhPr]Lys, [MorphAc]Lys, [MePipAc]Lys, [MeBipipAc]Lys, [4MePipBz]Lys, [MeMorphBz]Lys, [Me2NCBz]Lys, [mPEG4]Lys, [mPEG6]Lys, [mPEG8]Lys, [Bua]Lys, [Oct]Lys, [AdamantC]Lys, [Me3AdamantC]Lys, [AdamantPro]Lys, Ile, Lys, and iPrLys.
617. The agent of any one of the preceding Embodiments, wherein X⁸ is Ala.
618. The agent of any one of the preceding Embodiments, wherein X⁸ is selected from [lithocholate]-Lys, [lithocholate-PEG2]-Lys, [Me2NCBz]Lys, [Me3AdamantC-PEG2]-Lys, F2PipAbu, F2PipNva, MePpzAbu, MePpzAsn, MePpzNva, MorphAbu, MorphAsn, MorphNva, dAla, [1NapPr]Lys, [22PhPr]Lys, [35CF3PhPr]Lys, [4MePipBz]Lys, [AdamantC]Lys, [AdamantPro]Lys, [Bua]Lys, [Me3AdamantC]Lys, [Me3AdamantC]-Lys, [MeBipipAc]Lys, [MeMorphBz]Lys, [MePipAc]Lys, [MorphAc]Lys, [mPEG2]Lys, [mPEG4]Lys, [mPEG6]Lys, [mPEG8]Lys, [Oct]Lys, 3COOHF, Aad, Aib, Ala, Asp, Gln, Glu, Gly, Ile, iPrLys, Leu, Lys, nLeu, Phe, Ser, and Trp.
619. The agent of any one of the preceding Embodiments, wherein X⁸ is selected from [lithocholate]-Lys, [lithocholate-PEG2]-Lys, [Me2NCBz]Lys, [Me3AdamantC-PEG2]-Lys, F2PipAbu, F2PipNva, MePpzAbu, MePpzAsn, MePpzNva, MorphAbu, MorphAsn, MorphNva, and dAla.
620. The agent of any one of the preceding Embodiments, wherein X⁸ is selected from [1NapPr]Lys, [22PhPr]Lys, [35CF3PhPr]Lys, [4MePipBz]Lys, [AdamantC]Lys, [AdamantPro]Lys, [Bua]Lys, [Me3AdamantC]Lys, [Me3AdamantC]-Lys, [MeBipipAc]Lys, [MeMorphBz]Lys, [MePipAc]Lys, [MorphAc]Lys, [mPEG2]Lys, [mPEG4]Lys, [mPEG6]Lys, [mPEG8]Lys, [Oct]Lys, 3COOHF, Aad, Aib, Ala, Asp, Gln, Glu, Gly, Ile, iPrLys, Leu, Lys, nLeu, Phe, Ser, and Trp.
621. The agent of any one of the preceding Embodiments, wherein X⁹ is selected from Phe, 3F3MeF, 2Thi, 3Thi, 4F3MeF, 30MeF, 3MeF, 2MeF, 2NapA, 345FF, 34FF, 3FF, Asp, Cha, His, 2FurA, 2PyrA, 4AmPhe, 4FF, CypA, Gln, 1meH, 23FF, 2FF, 35FF, 3CBMF, 3ClF, 3meH, 3PyrA, 4CBMF, 4ClF, 4Thz, Ala, BztA, hPhe, hTyr, MeTyr, nLeu, 1NapA, 2CNF, 3CNF, 4CNF, 4MeF, Bip, DipA, npG, and Phg.
622. The agent of any one of the preceding Embodiments, wherein X⁹ is selected from [3Py]4SF, [CH₂NMe2]4SEF, [CH₂NMe2]TriAzDap, [CH₂Ppz]TriAzDap, [MorphCH2]TriAzDap, [SO2MorphCH2]TriAzDap, 2NH2F, 3CH₂NMe2F, 3CO2PhF, 3S02F, 4BrF, Cba, 1meH, 1NapA, 23FF, 2cbmf, 2ClF, 2CNF, 2FF, 2FurA, 2MeF, 2NapA, 2PyrA, 2Thi, 345FF, 34FF, 35FF, 3CBMF, 3ClF, 3CNF, 3COOHF, 3F3MeF, 3FF, 3MeF, 3meH, 30MeF, 3PyrA, 3Thi, 4AmPhe, 4CBMF, 4ClF, 4CNF, 4F3MeF, 4FF, 4MeF, 4Thz, Ala, Asp, Bip, BztA, Cha, CypA, DipA, Gln, His, hPhe, hTyr, MeTyr, nLeu, Npg, Phe, Phg, Ser, and Tyr.
623. The agent of any one of the preceding Embodiments, wherein X⁹ is selected from [3Py]4SF, [CH₂NMe2]4SEF, [CH₂NMe2]TriAzDap, [CH₂Ppz]TriAzDap, [MorphCH2]TriAzDap, [SO2MorphCH2]TriAzDap, 2NH2F, 3CH₂NMe2F, 3CO2PhF, 3S02F, 4BrF, and Cba.
624. The agent of any one of the preceding Embodiments, wherein X⁹ is selected from 1meH, 1NapA, 23FF, 2cbmf, 2ClF, 2CNF, 2FF, 2FurA, 2MeF, 2NapA, 2PyrA, 2Thi, 345FF, 34FF, 35FF, 3CBMF, 3ClF, 3CNF, 3COOHF, 3F3MeF, 3FF, 3MeF, 3meH, 30MeF, 3PyrA, 3Thi, 4AmPhe, 4CBMF, 4ClF, 4CNF, 4F3MeF, 4FF, 4MeF, 4Thz, Ala, Asp, Bip, BztA, Cha, CypA, DipA, Gln, His, hPhe, hTyr, MeTyr, nLeu, Npg, Phe, Phg, Ser, and Tyr.
625. The agent of any one of the preceding Embodiments, wherein X⁹ comprises a side chain which is or comprises an optionally substituted aromatic group.
626. The agent of any one of the preceding Embodiments, wherein X⁹ is —N(R^{a1})-L^{a1}-C(R^{a2})(R^{a3})-L^{a2}-C(O)—.
627. The agent of Embodiment 626, wherein R^{a1} is —H.
628. The agent of any one of Embodiments 626−627, wherein R^{a3} is —H.
629. The agent of any one of Embodiments 626−627, wherein R^{a3} is optionally substituted C₁₋₆ aliphatic.
630. The agent of any one of Embodiments 626−629, wherein L^{a1} is a covalent bond.
631. The agent of any one of Embodiments 626−630, wherein R^{a2} is -L^{a}-R, wherein R is or comprises an aromatic group.
632. The agent of Embodiment 631, wherein R is optionally substituted 6-10 membered aryl.
633. The agent of Embodiment 631, wherein R is optionally substituted phenyl.
634. The agent of Embodiment 631, wherein R is phenyl.
635. The agent of Embodiment 631, wherein R is optionally substituted naphthyl.
636. The agent of Embodiment 631, wherein R is naphthyl.
637. The agent of Embodiment 631, wherein R is optionally substituted 5-membered heteroaryl having 1-4 heteroatoms.
638. The agent of Embodiment 631, wherein R is optionally substituted 6-membered heteroaryl having 1-4 heteroatoms.
639. The agent of Embodiment 631, wherein R is optionally substituted 9-membered bicyclic heteroaryl having 1-4 heteroatoms.
640. The agent of Embodiment 631, wherein R is optionally substituted 10-membered bicyclic heteroaryl having 1-4 heteroatoms.
641. The agent of any one of Embodiments 637−640, wherein a heteroatom is nitrogen.
642. The agent of any one of Embodiments 637−641, wherein a heteroatom is oxygen.
643. The agent of any one of Embodiments 637−642, wherein a heteroatom is sulfur.
644. The agent of any one of Embodiments 637−640, wherein the heteroaryl has only one heteroatom.
645. The agent of Embodiment 644, wherein the heteroatom is nitrogen.
646. The agent of Embodiment 644, wherein the heteroatom is oxygen.
647. The agent of Embodiment 644, wherein the heteroatom is sulfur.
648. The agent of any one of Embodiments 631−647, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
649. The agent of any one of Embodiments 631−647, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
650. The agent of any one of Embodiments 631−647, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
651. The agent of Embodiment 648, wherein L^{a} is optionally substituted —(CH₂)ₙ— wherein n is 1, 2, 3, 4, 5, or 6.
652. The agent of Embodiment 648, wherein L^{a} is —(CH₂)ₙ— wherein n is 1, 2, 3, 4, 5, or 6.
653. The agent of Embodiment 648, wherein L^{a} is —CH₂—.
654. The agent of any one of the preceding Embodiments, wherein X⁹ comprises a side chain which is or comprises an optionally substituted aromatic group, wherein each optional substituent of the aromatic group is independently selected from halogen, —OR, —R, —N(R)₂, —C(O)N(R)₂, or —CN, wherein each R is independently —H, C₁₋₄ alkyl or haloalkyl, or -Ph.
655. The agent of any one of the preceding Embodiments, wherein X⁹ comprises a side chain which is or comprises an optionally substituted aromatic group, wherein each optional substituent of the aromatic group is independently selected from halogen, —OR, —R, —NH₂, —C(O)NH₂, -Ph, or —CN, wherein each R is independently C₁₋₄ alkyl or haloalkyl.
656. The agent of any one of the preceding Embodiments, wherein X⁹ comprises a side chain which is or comprises an optionally substituted aromatic group, wherein each optional substituent of the aromatic group is independently selected from halogen, —OR, —R, —NH₂, —C(O)NH₂, -Ph, or —CN, wherein each R is independently C₁₋₂ alkyl or haloalkyl.
657. The agent of any one of the preceding Embodiments, wherein X⁹ comprises a side chain which is or comprises an optionally substituted aromatic group, wherein each optional substituent of the aromatic group is independently selected from halogen, —OR, —R, NH₂, —C(O)NH₂, -Ph, or —CN, wherein each R is independently methyl optionally substituted with one or more halogen.
658. The agent of any one of the preceding Embodiments, wherein X⁹ comprises a side chain which is or comprises an optionally substituted aromatic group, wherein each optional substituent of the aromatic group is independently selected from —F, —OR, —CH₃, —NH₂, —C(O)NH₂, -Ph, or —CN, wherein each R is independently methyl optionally substituted with one or more —F.
659. The agent of any one of the preceding Embodiments, wherein X⁹ comprises a side chain which is or comprises an optionally substituted aromatic group, wherein each optional substituent of the aromatic group is independently selected from —F, —OR, —CH₃, —CF₃, —NH₂, —C(O)NH₂, -Ph, or —CN.
660. The agent of any one of the preceding Embodiments, wherein X⁹ comprises a side chain which is or comprises an optionally substituted aromatic group, wherein each optional substituent of the aromatic group is independently selected from halogen, —CH₃, —CF₃, or —CN.
661. The agent of any one of the preceding Embodiments, wherein X⁹ comprises a side chain which is or comprises an optionally substituted aromatic group optionally substituted at 2′-position.
662. The agent of any one of the preceding Embodiments, wherein X⁹ comprises a side chain which is or comprises an unsubstituted aromatic group.
663. The agent of any one of the preceding Embodiments, wherein X⁹ is Phe.
664. The agent of any one of the preceding Embodiments, wherein X¹⁰ is selected from Asn, Val, Gln, Leu, Thr, Ser, Phe, Ala, Hse, Cit, iPrLys, S7, S5, Cha, PyrS, S(Ome), [AzAc]Lys, nLeu, 2F3MeF, 3F3MeF, and 4F3MeF.
665. The agent of any one of the preceding Embodiments, wherein X¹⁰ is selected from [4MePpzPip]GlnR, [4Pippip]GlnR, [4PyPip]GlnR, [CH₂NMe2]TriAzDap, [CH₂Ppz]TriAzDap, [H4IAP]GlnR, [Me2NPrPip]GlnR, [Morph]GlnR, [MorphCH2]TriAzDap, [NHBn]GlnR, [Ppz]GlnR, [RDMAPyr]GlnR, [SO2MorphCH2]TriAzDap, [TfePpz]GlnR, 4BrF, AcLys, F2PipNva, Me2Asn, Me2Gln, MePpzAsn, MorphAsn, MorphGln, MorphNva, MeAsn, 2F3MeF, 3F3MeF, 4F3MeF, Abu, Ala, Arg, Asn, Cha, Cit, dSer, Gln, His, hLeu, Hse, iPrLys, Leu, Lys, nLeu, Npg, Phe, PyrS, PyrS2, R5, S(Ome), S5, S7, Ser, Thr, Trp, and Val.
666. The agent of any one of the preceding Embodiments, wherein X¹⁰ is selected from [4MePpzPip]GlnR, [4Pippip]GlnR, [4PyPip]GlnR, [CH₂NMe2]TriAzDap, [CH₂Ppz]TriAzDap, [H4IAP]GlnR, [Me2NPrPip]GlnR, [Morph]GlnR, [MorphCH2]TriAzDap, [NHBn]GlnR, [Ppz]GlnR, [RDMAPyr]GlnR, [SO2MorphCH2]TriAzDap, [TfePpz]GlnR, 4BrF, AcLys, F2PipNva, Me2Asn, Me2Gln, MePpzAsn, MorphAsn, MorphGln, MorphNva, and MeAsn.
667. The agent of any one of the preceding Embodiments, wherein X¹⁰ is selected from 2F3MeF, 3F3MeF, 4F3MeF, Abu, Ala, Arg, Asn, Cha, Cit, dSer, Gln, His, hLeu, Hse, iPrLys, Leu, Lys, nLeu, Npg, Phe, PyrS, PyrS2, R5, S(Ome), S5, S7, Ser, Thr, Trp, and Val.
668. The agent of any one of the preceding Embodiments, wherein X¹⁰ comprises a side chain comprising a polar group.
669. The agent of any one of the preceding Embodiments, wherein X¹⁰ comprises a side chain comprising —OH.
670. The agent of any one of the preceding Embodiments, wherein X¹⁰ is —N(R^{a1})-L^{a1}-C(R^{a2})(R^{a3})-L^{a2}-C(O)—.
671. The agent of Embodiment 670, wherein R^{a1} is —H.
672. The agent of any one of Embodiments 670−671, wherein R^{a3} is —H.
673. The agent of any one of Embodiments 670−671, wherein R^{a3} is optionally substituted C₁₋₆ aliphatic.
674. The agent of any one of Embodiments 670−673, wherein L^{a1} is a covalent bond.
675. The agent of any one of Embodiments 670−674, wherein L^{a2} is a covalent bond.
676. The agent of any one of Embodiments 670−675, wherein R^{a2} is -L″-R.
677. The agent of any one of Embodiments 670−675, wherein R^{a2} is -L″-Cy-R.
678. The agent of any one of Embodiments 676−677, wherein R is hydrogen or optionally substituted C₁₋₁₀ aliphatic.
679. The agent of any one of Embodiments 676−677, wherein R is optionally substituted C₁₋₁₀ aliphatic.
680. The agent of any one of Embodiments 676−677, wherein R is C₁₋₁₀ aliphatic.
681. The agent of any one of Embodiments 676−677, wherein R is C₁₋₁₀ alkyl.
682. The agent of any one of Embodiments 676−677, wherein R is optionally substituted phenyl.
683. The agent of any one of Embodiments 670−675, R^{a2} is -L″—C(O)N(R′)₂.
684. The agent of any one of Embodiments 670−675, R^{a2} is -L″—OH.
685. The agent of any one of Embodiments 670−684, wherein L″ is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
686. The agent of any one of Embodiments 670−684, wherein L″ is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
687. The agent of any one of Embodiments 670−684, wherein L″ is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
688. The agent of any one of Embodiments 670−684, wherein L″ is optionally substituted —(CH₂)ₙ— wherein n is 1, 2, 3, 4, 5, or 6.
689. The agent of any one of Embodiments 670−684, wherein L″ is —(CH₂)ₙ— wherein n is 1, 2, 3, 4, 5, or 6.
690. The agent of any one of the preceding Embodiments, wherein X¹⁰ is Leu, Thr or Hse.
691. The agent of any one of Embodiments 1−664, wherein X¹⁰ comprises a side chain comprising an amide group.
692. The agent of any one of Embodiments 1−664, wherein X¹⁰ comprises a hydrophobic side chain.
693. The agent of any one of Embodiments 1−664, wherein X¹⁰ comprises a hydrocarbon side chain.
694. The agent of Embodiment 693, wherein X¹⁰ is Leu.
695. The agent of any one of Embodiments 1−664, wherein X¹⁰ comprises a side chain comprising an optionally substituted aromatic group.
696. The agent of Embodiment 695, wherein the aromatic group is optionally substituted phenyl.
697. The agent of Embodiment 695, wherein X¹⁰ is Phe.
698. The agent of any one of the preceding Embodiments, wherein X¹¹ is selected from S8, PyrS2, PyrS, S7, PyrS3, SeN, Ala, Az, Phe, S4, S6, SdN, S10, S5, SgN, and PyrS1.
699. The agent of any one of the preceding Embodiments, wherein X¹¹ is selected from Az2, Az3, PyrR2, PyrS4, SeNc5, SPip1, SPip2, SPip3, Aib, Ala, Az, Leu, Phe, PyrS, PyrS1, PyrS2, PyrS3, S10, S4, S5, S6, S7, S8, SdN, SeN, and SgN.
700. The agent of any one of the preceding Embodiments, wherein X″ is selected from Az2, Az3, PyrR2, PyrS4, SeNc5, SPip1, SPip2, and SPip3.
701. The agent of any one of the preceding Embodiments, wherein X″ is selected from Aib, Ala, Az, Leu, Phe, PyrS, PyrS1, PyrS2, PyrS3, S10, S4, S5, S6, S7, S8, SdN, SeN, and SgN.
702. The agent of any one of the preceding Embodiments, wherein X″ is stapled with X⁴.
703. The agent of Embodiment 702, wherein X″ is PyrS2.
704. The agent of Embodiment 702, wherein X″ is PyrS3.
705. The agent of Embodiment 702, wherein X″ is S8.
706. The agent of any one of Embodiments 1−698, wherein X″ is not stapled.
707. The agent of Embodiment 705, wherein X″ is Ala or Phe.
708. The agent of any one of the preceding Embodiments, wherein X² is selected from Phe, 3Thi, 2ClF, 3FF, 20MeF, 2FF, Pff, Asp, 2CBMF, 3ClF, 3F3MeF, 1NapA, 2NapA, 2PyrA, 4CBMF, 4COOHF, 4F3MeF, Tyr, 2BrF, 2F3MeF, 2Thi, 4PyrA, Cha, CypA, hPhe, Trp, dPhe, [Acryl]Dap, 1meH, 23FF, 2MeF, 34FF, 30MeF, 3PyrA, 4ClF, 4CNF, Ala, Glu, His, 2CNF, 2N02F, 35FF, 3CBMF, 3CNF, 3MeF, 3meH, 4FF, 4MeF, 4Thz, Asn, BztA, and hTyr.
709. The agent of any one of the preceding Embodiments, wherein X² is selected from [CyPr]-3SF, [Ph]3SF, [Ph]-3SF, 3BrF, 3CBMF, Cba, [Acryl]Dap, 1meH, 1NapA, 23FF, 2BrF, 2CBMF, 2ClF, 2CNF, 2F3MeF, 2FF, 2MeF, 2NapA, 2N02F, 20MeF, 2pyrA, 2Thi, 34FF, 35FF, 3ClF, 3CNF, 3F3MeF, 3FF, 3MeF, 3meH, 30MeF, 3PyrA, 3thi, 4CBMF, 4ClF, 4CNF, 4COOHF, 4F3MeF, 4FF, 4MeF, 4PyrA, 4Thz, Ala, Asn, Asp, BztA, Cha, CypA, dPhe, Gln, Glu, His, hPhe, hTyr, Leu, Npg, Pff, Phe, PyrS2, Trp, and Tyr.
710. The agent of any one of the preceding Embodiments, wherein X² is selected from [CyPr]-3SF, [Ph]3SF, [Ph]-3SF, 3BrF, 3CBMF, and Cba.
711. The agent of any one of the preceding Embodiments, wherein X² is selected from [Acryl]Dap, 1meH, 1NapA, 23FF, 2BrF, 2CBMF, 2ClF, 2CNF, 2F3MeF, 2FF, 2MeF, 2NapA, 2N02F, 20MeF, 2pyrA, 2Thi, 34FF, 35FF, 3ClF, 3CNF, 3F3MeF, 3FF, 3MeF, 3meH, 30MeF, 3PyrA, 3thi, 4CBMF, 4ClF, 4CNF, 4COOHF, 4F3MeF, 4FF, 4MeF, 4PyrA, 4Thz, Ala, Asn, Asp, BztA, Cha, CypA, dPhe, Gln, Glu, His, hPhe, hTyr, Leu, Npg, Pff, Phe, PyrS2, Trp, and Tyr.
712. The agent of any one of the preceding Embodiments, wherein X² comprises a side chain which is or comprises an optionally substituted aromatic group.
713. The agent of any one of the preceding Embodiments, wherein X² is —N(R^{a1})-Lal-C(R^{a2})(R^{a3})-L^{a2}-C(O)—.
714. The agent of Embodiment 713, wherein R^{a1} is —H.
715. The agent of any one of Embodiments 713-714, wherein R^{a3} is —H.
716. The agent of any one of Embodiments 713-714, wherein R^{a3} is optionally substituted C₁₋₆ aliphatic.
717. The agent of any one of Embodiments 713-716, wherein L^{a1} is a covalent bond.
718. The agent of any one of Embodiments 713-717, wherein R^{a2} is -L^{a}-R, wherein R is or comprises an aromatic group.
719. The agent of Embodiment 718, wherein R is optionally substituted 6-10 membered aryl
720. The agent of Embodiment 718, wherein R is optionally substituted phenyl
721. The agent of Embodiment 718, wherein R is phenyl
722. The agent of Embodiment 718, wherein R is optionally substituted naphthyl
723. The agent of Embodiment 718, wherein R is naphthyl
724. The agent of Embodiment 718, wherein R is optionally substituted 5-membered heteroaryl having 1-4 heteroatoms
725. The agent of Embodiment 718, wherein R is optionally substituted 6-membered heteroaryl having 1-4 heteroatoms
726. The agent of Embodiment 718, wherein R is optionally substituted 9-membered bicyclic heteroaryl having 1-4 heteroatoms
727. The agent of Embodiment 718, wherein R is optionally substituted 10-membered bicyclic heteroaryl having 1-4 heteroatoms
728. The agent of any one of Embodiments 724-727, wherein a heteroatom is nitrogen
729. The agent of any one of Embodiments 724-728, wherein a heteroatom is oxygen
730. The agent of any one of Embodiments 724-729, wherein a heteroatom is sulfur
731. The agent of any one of Embodiments 724-727, wherein the heteroaryl has only one heteroatom
732. The agent of Embodiment 731, wherein the heteroatom is nitrogen.
733. The agent of Embodiment 731, wherein the heteroatom is oxygen.
734. The agent of Embodiment 731, wherein the heteroatom is sulfur.
735. The agent of any one of Embodiments 718-734, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
736. The agent of any one of Embodiments 718-734, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
737. The agent of any one of Embodiments 718-734, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
738. The agent of Embodiment 735, wherein L^{a} is optionally substituted —(CH₂)ₙ— wherein n is 1, 2, 3, 4, 5, or 6.
739. The agent of Embodiment 735, wherein L^{a} is —(CH₂)ₙ— wherein n is 1, 2, 3, 4, 5, or 6.
740. The agent of Embodiment 735, wherein L^{a} is —CH₂—.
741. The agent of any one of the preceding Embodiments, wherein X² comprises a side chain which is or comprises an optionally substituted aromatic group, wherein each optional substituent of the aromatic group is independently selected from halogen, —OR, —R, —N(R)₂, —C(O)N(R)₂, or —CN, wherein each R is independently —H, C₁₋₄ alkyl or haloalkyl, or -Ph.
742. The agent of any one of the preceding Embodiments, wherein X² comprises a side chain which is or comprises an optionally substituted aromatic group, wherein each optional substituent of the aromatic group is independently selected from halogen, —OR, —R, —NH₂, —C(O)NH₂, -Ph, or —CN, wherein each R is independently C₁₋₄ alkyl or haloalkyl.
743. The agent of any one of the preceding Embodiments, wherein X² comprises a side chain which is or comprises an optionally substituted aromatic group, wherein each optional substituent of the aromatic group is independently selected from halogen, —OR, —R, —NH₂, —C(O)NH₂, -Ph, or —CN, wherein each R is independently C₁₋₂ alkyl or haloalkyl.
744. The agent of any one of the preceding Embodiments, wherein X² comprises a side chain which is or comprises an optionally substituted aromatic group, wherein each optional substituent of the aromatic group is independently selected from halogen, —OR, —R, NH₂, —C(O)NH₂, -Ph, or —CN, wherein each R is independently methyl optionally substituted with one or more halogen.
745. The agent of any one of the preceding Embodiments, wherein X² comprises a side chain which is or comprises an optionally substituted aromatic group, wherein each optional substituent of the aromatic group is independently selected from —F, —OR, —CH₃, —NH₂, —C(O)NH₂, -Ph, or —CN, wherein each R is independently methyl optionally substituted with one or more —F.
746. The agent of any one of the preceding Embodiments, wherein X² comprises a side chain which is or comprises an optionally substituted aromatic group, wherein each optional substituent of the aromatic group is independently selected from —F, —OR, —CH₃, —CF₃, —NH₂, —C(O)NH₂, -Ph, or —CN.
747. The agent of any one of the preceding Embodiments, wherein X² comprises a side chain which is or comprises an optionally substituted aromatic group, wherein each optional substituent of the aromatic group is independently selected from halogen, —CH₃, —CF₃, or —CN.
748. The agent of any one of the preceding Embodiments, wherein X² comprises a side chain which is or comprises an optionally substituted aromatic group optionally substituted at 2′-position.
749. The agent of any one of the preceding Embodiments, wherein X² comprises a side chain which is or comprises an unsubstituted aromatic group.
750. The agent of any one of Embodiments 741-749, wherein the aromatic group is a 5-membered heteroaryl group.
751. The agent of any one of the preceding Embodiments, wherein X² is 3Thi.
752. The agent of any one of Embodiments 741-749, wherein the aromatic group is a phenyl group.
753. The agent of any one of Embodiment 752, wherein X² is 2ClF.
754. The agent of any one of Embodiment 752, wherein X² is Phe.
755. The agent of any one of Embodiment 752, wherein X² is Phe wherein the phenyl is 2′-substituted.
756. The agent of any one of Embodiment 752, wherein X² is 2FF, 2ClF, 2BrF, 2F3MeF, 2MeF, or 2CNF.
757. The agent of any one of the preceding Embodiments, wherein X¹³ is selected from BztA, Trp, 2NapA, 1NapA, WCHO, 5CpW, 5FW, Ala, aMeW, H2Trp, His, Phe, 23FF, 34FF, 340MeF, 1MeW, 5CF3W, 5ClW, 5MeOW, 6ClW, 6F1NapA, 7F1NapA, 7FW, Bip, and Qui.
758. The agent of any one of the preceding Embodiments, wherein X¹³ is selected from 2F3MeW, 34ClF, 34MeF, 3Br4FF, 3BrF, 4ClBztA, 4ClW, 4FW, 5IndA, 7AzaW, 7ClBztA, 7FBztA, Cba, RbMe2NapA, RbMeBztA, SbMe2NapA, SbMeBztA, 1MeW, 1NapA, 23FF, 2F3MeF, 2NapA, 34FF,
340MeF, 3ClF, 3Thi, 4ClF, 5CF3W, 5ClW, 5CpW, 5FW, 5MeOW, 6ClW, 6F1NapA, 7F1NapA, 7FW, Ala, aMeW, Bip, BztA, Cha, H2Trp, His, Phe, PyrS2, Qui, Trp, Tyr, and WCHO.
759. The agent of any one of the preceding Embodiments, wherein X¹³ is selected from 2F3MeW, 34ClF, 34MeF, 3Br4FF, 3BrF, 4ClBztA, 4ClW, 4FW, 5IndA, 7AzaW, 7ClBztA, 7FBztA, Cba, RbMe2NapA, RbMeBztA, SbMe2NapA, and SbMeBztA.
760. The agent of any one of the preceding Embodiments, wherein X¹³ is selected from 1MeW, 1NapA, 23FF, 2F3MeF, 2NapA, 34FF, 340MeF, 3ClF, 3Thi, 4ClF, 5CF3W, 5ClW, 5CpW, 5FW, 5MeOW, 6ClW, 6F1NapA, 7F1NapA, 7FW, Ala, aMeW, Bip, BztA, Cha, H2Trp, His, Phe, PyrS2, Qui, Trp, Tyr, and WCHO.
761. The agent any one of the preceding Embodiments, wherein the side chain of X¹³ comprises an optionally substituted aromatic group.
762. The agent of any one of the preceding Embodiments, wherein X¹³ is —N(R^{a1})-L^{a1}-C(R^{a2})(R^{a3})-L^{a2}-C(O)—.
763. The agent of Embodiment 762, wherein R^{a1} is —H.
764. The agent of any one of Embodiments 762-763, wherein R^{a3} is —H.
765. The agent of any one of Embodiments 762-763, wherein R^{a3} is optionally substituted C₁₋₆ aliphatic.
766. The agent of any one of Embodiments 762-765, wherein L^{a1} is a covalent bond
767. The agent of any one of Embodiments 762-766, wherein R^{a2} is -L^{a}-R, wherein R is or comprises an aromatic group.
768. The agent of Embodiment 767, wherein R is optionally substituted 6-10 membered aryl.
769. The agent of Embodiment 767, wherein R is optionally substituted phenyl.
770. The agent of Embodiment 767, wherein R is phenyl.
771. The agent of Embodiment 767, wherein R is optionally substituted naphthyl.
772. The agent of Embodiment 767, wherein R is naphthyl.
773. The agent of Embodiment 767, wherein R is optionally substituted 5-membered heteroaryl having 1-4 heteroatoms.
774. The agent of Embodiment 767, wherein R is optionally substituted 6-membered heteroaryl having 1-4 heteroatoms.
775. The agent of Embodiment 767, wherein R is optionally substituted 9-membered bicyclic heteroaryl having 1-4 heteroatoms.
776. The agent of Embodiment 767, wherein R is optionally substituted 10-membered bicyclic heteroaryl having 1-4 heteroatoms.
777. The agent of any one of Embodiments 773-776, wherein a heteroatom is nitrogen.
778. The agent of any one of Embodiments 773-776, wherein a heteroatom is oxygen.
779. The agent of any one of Embodiments 773-776, wherein a heteroatom is sulfur.
780. The agent of any one of Embodiments 773-776, wherein the heteroaryl has only one heteroatom.
781. The agent of Embodiment 780, wherein the heteroatom is nitrogen.
782. The agent of Embodiment 780, wherein the heteroatom is oxygen.
783. The agent of Embodiment 780, wherein the heteroatom is sulfur.
784. The agent of any one of Embodiments 767-783, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
785. The agent of any one of Embodiments 767-783, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
786. The agent of any one of Embodiments 767-783, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
787. The agent of Embodiment 784, wherein L^{a} is optionally substituted —(CH₂)ₙ— wherein n is 1, 2, 3, 4, 5, or 6.
788. The agent of Embodiment 784, wherein L^{a} is —(CH₂)ₙ— wherein n is 1, 2, 3, 4, 5, or 6.
789. The agent of Embodiment 784, wherein L^{a} is —CH₂—.
790. The agent any one of the preceding Embodiments, wherein the side chain of X¹³ comprises an optionally substituted 8-10 membered bicyclic aromatic group.
791. The agent any one of the preceding Embodiments, wherein the side chain of X¹³ comprises an optionally substituted 9-membered bicyclic heteroaryl group having 1-3 heteroatoms.
792. The agent of any one of the preceding Embodiments, wherein X¹³ is BtzA.
793. The agent of any one of Embodiments 1-791, wherein X¹³ is Trp.
794. The agent of any one of Embodiments 1-791, wherein X¹³ is 1NapA.
795. The agent of any one of Embodiments 1-791, wherein X¹³ is 2NapA.
796. The agent of any one of the preceding Embodiments, wherein p14 is 1.
797. The agent of any one of the preceding Embodiments, wherein X¹⁴ is selected from Gln, His, Ser, dThr, Thr, Ala, Hse, Asn, Leu, Aib, Alaol, Throl, Leuol, dAsn, dGln, dHis, Tyr, [AzAc]Lys, 1MeH, 3MeH, 4TriA, dSer, NMeHis, NMeS, Pro, Trp, Val, Lys, MorphAla, 2FurA, Abu, Arg, Dab, iPrLys, Phe, Pheol, and Prool.
798. The agent of any one of the preceding Embodiments, wherein X¹⁴ is selected from [3C]TriAzLys, [4F3CPip]GlnR, [4MePpzPip]GlnR, [4Pippip]GlnR, [AcPpz]GlnR, [bismethoxyethylamine]GlnR, [Me2NPrPip]GlnR, [Morph]GlnR, [NHEt]GlnR, [NMe2]GlnR, [Pip]GlnR, [PropynPEG14]Lys, [RDMAPyr]GlnR, [TfePpz]GlnR, AcLys, BnBoroleK, F2PipNva, GlnR, Me2Asn, Me2Gln, MePpzAsn, Met20, MorphAsn, MorphGln, MorphNva, dAla, MeAsn, 1MeH, 2cbmf, 2F3MeF, 2FurA, 3cbmf, 3MeH, 4TriA, Abu, Aib, Ala, Alaol, Arg, Asn, Asp, BztA, Cha, Dab, dAsn, dGln, dHis, dSer, dThr, Gln, His, Hse, iPrLys, Leu, Leuol, Lys, MorphAla, NMeHis, NMeS, Npg, Phe, Pheol, Pro, Prool, PyrS2, S5, Ser, Thr, Throl, Trp, Tyr, and Val.
799. The agent of any one of the preceding Embodiments, wherein X¹⁴ is selected from [3C]TriAzLys, [4F3CPip]GlnR, [4MePpzPip]GlnR, [4Pippip]GlnR, [AcPpz]GlnR, [bismethoxyethylamine]GlnR, [Me2NPrPip]GlnR, [Morph]GlnR, [NHEt]GlnR, [NMe2]GlnR, [Pip]GlnR, [PropynPEG14]Lys, [RDMAPyr]GlnR, [TfePpz]GlnR, AcLys, BnBoroleK, F2PipNva, GlnR, Me2Asn, Me2Gln, MePpzAsn, Met20, MorphAsn, MorphGln, MorphNva, dAla, and MeAsn.
800. The agent of any one of the preceding Embodiments, wherein X¹⁴ is selected from 1MeH, 2cbmf, 2F3MeF, 2FurA, 3cbmf, 3MeH, 4TriA, Abu, Aib, Ala, Alaol, Arg, Asn, Asp, BztA, Cha, Dab, dAsn, dGln, dHis, dSer, dThr, Gln, His, Hse, iPrLys, Leu, Leuol, Lys, MorphAla, NMeHis, NMeS, Npg, Phe, Pheol, Pro, Prool, PyrS2, S5, Ser, Thr, Throl, Trp, Tyr, and Val.
801. The agent of any one of the preceding Embodiments, wherein X¹⁴ comprises a side chain comprising a polar group.
802. The agent of any one of the preceding Embodiments, wherein X¹⁴ is —N(R^{a1})-L^{a1}-C(R^{a2})(R^{a3})-L^{a2}-C(O)—.
803. The agent of Embodiment 802, wherein R^{a1} is —H.
804. The agent of any one of Embodiments 802-803, wherein R^{a3} is —H.
805. The agent of any one of Embodiments 802-803, wherein R^{a3} is optionally substituted C₁₋₆ aliphatic.
806. The agent of any one of Embodiments 802-805, wherein L^{a1} is a covalent bond.
807. The agent of any one of Embodiments 802-806, wherein L^{a2} is a covalent bond.
808. The agent of any one of Embodiments 802-807, wherein R^{a2} is -L″-R.
809. The agent of any one of Embodiments 802-807, wherein R^{a2} is -L″-Cy-R.
810. The agent of any one of Embodiments 802-807, wherein R^{a2} is -L″—C(O)OR.
811. The agent of any one of Embodiments 802-807, wherein R^{a2} is -L″—C(O)N(R′)₂.
812. The agent of any one of Embodiments 802-807, wherein R^{a2} is -L″—C(O)N(R)₂.
813. The agent of any one of Embodiments 804-812, wherein R is hydrogen or optionally substituted C₁₋₁₀ aliphatic.
814. The agent of any one of Embodiments 804-812, wherein R is hydrogen.
815. The agent of any one of Embodiments 804-812, wherein R is optionally substituted C₁₋₁₀ aliphatic.
816. The agent of any one of Embodiments 804-812, wherein R is C₁₋₁₀ aliphatic.
817. The agent of any one of Embodiments 804-812, wherein R is C₁₋₁₀ alkyl.
818. The agent of any one of Embodiments 802-807, wherein R^{a2} is -L″—OH.
819. The agent of any one of Embodiments 802-818, wherein L″ is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
820. The agent of any one of Embodiments 802-818, wherein L″ is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
821. The agent of any one of Embodiments 802-818, wherein L″ is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
822. The agent of any one of Embodiments 802-818, wherein L″ is optionally substituted —(CH₂)ₙ— wherein n is 1, 2, 3, 4, 5, or 6.
823. The agent of any one of Embodiments 802-818, wherein L″ is —(CH₂)ₙ— wherein n is 1, 2, 3, 4, 5, or 6.
824. The agent of any one of the preceding Embodiments, wherein X¹⁴ comprises a side chain comprising —OH.
825. The agent of any one of the preceding Embodiments, wherein X¹⁴ comprises a side chain comprising an amide group.
826. The agent of any one of the preceding Embodiments, wherein X¹⁴ is Gln.
827. The agent of any one of Embodiments 1-792, wherein p14 is 0.
828. The agent of any one of the preceding Embodiments, wherein p15 is 1.
829. The agent of any one of the preceding Embodiments, wherein X¹⁵ is —N(R^{a1})-L^{a1}-C(R^{a2})(R^{a3})-L^{a2}-C(O)—.
830. The agent of Embodiment 829, wherein R^{a1} is —H.
831. The agent of any one of Embodiments 829-830, wherein R^{a3} is —H.
832. The agent of any one of Embodiments 829-830, wherein R^{a3} is optionally substituted C₁₋₆ aliphatic.
833. The agent of any one of Embodiments 829-832, wherein L^{a1} is a covalent bond.
834. The agent of any one of Embodiments 829-833, wherein L^{a2} is a covalent bond.
835. The agent of any one of Embodiments 829-834, wherein R^{a2} is -L″-R.
836. The agent of any one of Embodiments 829-834, wherein R^{a2} is -L″-Cy-R.
837. The agent of any one of Embodiments 829-834, wherein R^{a2} is -L″—C(O)OR.
838. The agent of any one of Embodiments 829-834, wherein R^{a2} is -L″—C(O)N(R′)₂.
839. The agent of any one of Embodiments 829-834, wherein R^{a2} is -L″—C(O)N(R)₂.
840. The agent of any one of Embodiments 835-839, wherein R is hydrogen or optionally substituted C₁₋₁₀ aliphatic.
841. The agent of any one of Embodiments 835-839, wherein R is hydrogen.
842. The agent of any one of Embodiments 835-839, wherein R is optionally substituted C₁₋₁₀ aliphatic.
843. The agent of any one of Embodiments 835-839, wherein R is C₁₋₁₀ aliphatic.
844. The agent of any one of Embodiments 835-839, wherein R is C₁₋₁₀ alkyl.
845. The agent of any one of Embodiments 829-834, wherein R2 is -L″—OH.
846. The agent of any one of Embodiments 829-845, wherein L″ is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
847. The agent of any one of Embodiments 829-845, wherein L″ is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
848. The agent of any one of Embodiments 829-845, wherein L″ is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
849. The agent of any one of Embodiments 829-845, wherein L″ is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
850. The agent of any one of Embodiments 829-845, wherein L″ is —(CH₂)ₙ— wherein n is 1, 2, 3, 4, 5, or 6.
851. The agent of any one of the preceding Embodiments, wherein X⁵ is selected from 6AmHex, 6AzHex, Aib, Ala, dAla, dIle, Ile, and Lys.
852. The agent of any one of the preceding Embodiments, wherein X⁵ is selected from [1Napc]Lys, [1NapPr]Lys, [22PhPr]Lys, [2Napc]Lys, [35CF3PhPr]Lys, [4MePipBz]Lys, [AdamantC]Lys, [AzAc]Lys, [Bua]Lys, [Me2NCBz]Lys, [Me3AdamantC]Lys, [MeBipipAc]Lys, [MeMorphBz]Lys, [MePipAc]Lys, [MorphAc]Lys, [mPEG2]Lys, [mPEG4]Lys, [mPEG6]Lys, [mPEG8]Lys, [Oct]Lys, [PropynPEG1]Lys, [PropynPEG2]Lys, [PropynPEG3]Lys, [PropynPEG4]Lys, 6AmHex, 6AzHex, Aib, Ala, dAla, dIle, Ile, and Lys.
853. The agent of any one of the preceding Embodiments, wherein X⁵ is selected from [3C]TriAzdLys, [3C]TriAzLys, [lithocholate]-Lys, [lithocholate-PEG2]-Lys, [Me2NCBz]Lys, [Me3AdamantC-PEG2]-Lys, [PropynPEG14]Lys, dAla, dIle, [1Napc]Lys, [1NapPr]Lys, [22PhPr]Lys, [2Napc]Lys, [35CF3PhPr]Lys, [4MePipBz]Lys, [AdamantC]Lys, [Bua]Lys, [Me3AdamantC]Lys, [Me3AdamantC]-Lys, [MeBipipAc]Lys, [MeMorphBz]Lys, [MePipAc]Lys, [MorphAc]Lys, [mPEG2]Lys, [mPEG4]Lys, [mPEG6]Lys, [mPEG8]Lys, [Oct]Lys, [PropynPEG1]Lys, [PropynPEG2]Lys, [PropynPEG3]Lys, [PropynPEG4]Lys, 6AmHex, Aib, Ala, Alaol, BztA, Gln, Ile, Leuol, Leu, Lys, NEt2, NHBn, NHCyHe, NHCyPr, NHEt, OH, Phe, Pheol, Prool, Throl, and Val.
854. The agent of any one of the preceding Embodiments, wherein X⁵ is selected from [3C]TriAzdLys, [3C]TriAzLys, [lithocholate]-Lys, [lithocholate-PEG2]-Lys, [Me2NCBz]Lys, [Me3AdamantC-PEG2]-Lys, [PropynPEG14]Lys, dAla, and dIle.
855. The agent of any one of the preceding Embodiments, wherein X⁵ is selected from [1Napc]Lys, [1NapPr]Lys, [22PhPr]Lys, [2Napc]Lys, [35CF3PhPr]Lys, [4MePipBz]Lys, [AdamantC]Lys, [Bua]Lys, [Me3AdamantC]Lys, [Me3AdamantC]-Lys, [MeBipipAc]Lys, [MeMorphBz]Lys, [MePipAc]Lys, [MorphAc]Lys, [mPEG2]Lys, [mPEG4]Lys, [mPEG6]Lys, [mPEG8]Lys, [Oct]Lys, [PropynPEG1]Lys, [PropynPEG2]Lys, [PropynPEG3]Lys, [PropynPEG4]Lys, 6AmHex, Aib, Ala, Alaol, BztA, Gln, Ile, Leuol, Leu, Lys, NEt2, NHBn, NHCyHe, NHCyPr, NHEt, OH, Phe, Pheol, Prool, Throl, and Val.
856. The agent of any one of the preceding Embodiments, wherein X⁵ comprises a hydrophobic side chain.
857. The agent of any one of the preceding Embodiments, wherein the side chain of X⁵ is C₁₋₁₀ alkyl.
858. The agent of any one of Embodiments 1-852, wherein X⁵ is optionally substituted or labeled Lys.
859. The agent of any one of Embodiments 1-827, wherein p15 is 0.
860. The agent of any one of the preceding Embodiments, wherein p16 is 1.
861. The agent of any one of the preceding Embodiments, wherein X¹⁶ is —N(R^{a1})-L^{a1}-C(R^{a2})(R^{a3})-L^{a2}-C(O)—.
862. The agent of Embodiment 861, wherein R^{a1} is —H.
863. The agent of any one of Embodiments 861-862, wherein R^{a3} is —H.
864. The agent of any one of Embodiments 861-862, wherein R^{a3} is optionally substituted C₁₋₆ aliphatic.
865. The agent of any one of Embodiments 861-864, wherein L^{a1} is a covalent bond.
866. The agent of any one of Embodiments 861-865, wherein L^{a2} is a covalent bond.
867. The agent of any one of Embodiments 861-866, wherein R^{a2} is -L″-R.
868. The agent of any one of Embodiments 861-866, wherein R^{a2} is -L″-Cy-R.
869. The agent of any one of Embodiments 861-866, wherein R^{a2} is -L″—C(O)OR.
870. The agent of any one of Embodiments 861-866, wherein R^{a2} is -L″—C(O)N(R′)₂.
871. The agent of any one of Embodiments 861-866, wherein R^{a2} is -L″—C(O)N(R)₂.
872. The agent of any one of Embodiments 867-871, wherein R is hydrogen or optionally substituted C₁₋₁₀ aliphatic.
873. The agent of any one of Embodiments 867-871, wherein R is hydrogen.
874. The agent of any one of Embodiments 867-871, wherein R is optionally substituted C₁₋₁₀ aliphatic.
875. The agent of any one of Embodiments 867-871, wherein R is C₁₋₁₀ aliphatic.
876. The agent of any one of Embodiments 867-871, wherein R is C₁₋₁₀ alkyl.
877. The agent of any one of Embodiments 861-866, wherein R^{a2} is -L″—OH.
878. The agent of any one of Embodiments 861-877, wherein L″ is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
879. The agent of any one of Embodiments 861-877, wherein L″ is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
880. The agent of any one of Embodiments 861-877, wherein L″ is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
881. The agent of any one of Embodiments 861-877, wherein L″ is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
882. The agent of any one of Embodiments 861-877, wherein L″ is —(CH₂)ₙ— wherein n is 1, 2, 3, 4, 5, or 6.
883. The agent of any one of the preceding Embodiments, wherein X¹⁶ is selected from Ala and dAla.
884. The agent of any one of the preceding Embodiments, wherein X¹⁶ is selected from Cbg, Cpg, CyLeu, dLeu, dAla, Aib, Ala, Arg, Asn, dGln, dThr, Gln, Ile, Leu, nLeu, Phe, Ser, Thr, Trp, Tyr, and Val.
885. The agent of any one of the preceding Embodiments, wherein X¹⁶ is selected from Cbg, Cpg, CyLeu, dLeu, and dAla.
886. The agent of any one of the preceding Embodiments, wherein X¹⁶ is selected from Aib, Ala, Arg, Asn, dGln, dThr, Gln, Ile, Leu, nLeu, Phe, Ser, Thr, Trp, Tyr, and Val.
887. The agent of any one of Embodiments 1-859, wherein p16 is 0.
888. The agent of any one of the preceding Embodiments, wherein p17 is 1.
889. The agent of any one of the preceding Embodiments, wherein X⁷ is —N(R^{a1})-L^{a1}-C(R^{a2})(R^{a3})-L^{a2}-C(O)
890. The agent of Embodiment 889, wherein R^{a1} is —H.
891. The agent of any one of Embodiments 889-890, wherein R^{a3} is —H.
892. The agent of any one of Embodiments 889-890, wherein R^{a3} is optionally substituted C₁−6 aliphatic.
893. The agent of any one of Embodiments 889-892, wherein L^{a1} is a covalent bond.
894. The agent of any one of Embodiments 889-893, wherein L^{a2} is a covalent bond.
895. The agent of any one of Embodiments 889-894, wherein R^{a2} is -L″-R.
896. The agent of any one of Embodiments 889-894, wherein R^{a2} is -L″-Cy-R.
897. The agent of any one of Embodiments 889-894, wherein R^{a2} is -L″—C(O)OR.
898. The agent of any one of Embodiments 889-894, wherein R^{a2} is -L″—C(O)N(R′)₂.
899. The agent of any one of Embodiments 889-894, wherein R^{a2} is -L″—C(O)N(R)₂.
900. The agent of any one of Embodiments 895-899, wherein R is hydrogen or optionally substituted C₁₋₁₀ aliphatic.
901. The agent of any one of Embodiments 895-899, wherein R is hydrogen.
902. The agent of any one of Embodiments 895-899, wherein R is optionally substituted C₁₋₁₀ aliphatic.
903. The agent of any one of Embodiments 895-899, wherein R is C₁₋₁₀ aliphatic.
904. The agent of any one of Embodiments 895-899, wherein R is C₁₋₁₀ alkyl.
905. The agent of any one of Embodiments 889-894, wherein R^{a2} is -L″—OH.
906. The agent of any one of Embodiments 889-905, wherein L″ is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
907. The agent of any one of Embodiments 889-905, wherein L″ is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
908. The agent of any one of Embodiments 889-905, wherein L″ is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
909. The agent of any one of Embodiments 889-905, wherein L″ is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
910. The agent of any one of Embodiments 889-905, wherein L″ is —(CH₂)ₙ— wherein n is 1, 2, 3, 4, 5, or 6.
911. The agent of any one of the preceding Embodiments, wherein X⁷ is selected from Ala and dAla.
912. The agent of any one of Embodiments 1-859, wherein p17 is 0.
913. The agent of any one of the preceding Embodiments, wherein p18 is 1.
914. The agent of any one of the preceding Embodiments, wherein X¹⁸ comprises a hydrophobic side chain.
915. The agent of any one of the preceding Embodiments, wherein X¹s is selected from Aib, Ala, and Leu.
916. The agent of any one of the preceding Embodiments, wherein p18 is 0.
917. The agent of any one of the preceding Embodiments, wherein p19 is 1.
918. The agent of any one of the preceding Embodiments, wherein X¹⁹ comprises a hydrophobic side chain.
919. The agent of any one of the preceding Embodiments, wherein X¹⁹ is selected from Aib, Ala, and Leu.
920. The agent of any one of the preceding Embodiments, wherein p19 is 0.
921. The agent of any one of the preceding Embodiments, wherein p20 is 1.
922. The agent of any one of the preceding Embodiments, wherein X²⁰ comprises a hydrophobic side chain.
923. The agent of any one of the preceding Embodiments, wherein X²⁰ is selected from Aib, Ala, and Leu.
924. The agent of any one of the preceding Embodiments, wherein p20 is 0.
925. The agent of any one of the preceding Embodiments, wherein p21 is 1.
926. The agent of any one of the preceding Embodiments, wherein X²¹ comprises a hydrophobic side chain.
927. The agent of any one of the preceding Embodiments, wherein X²¹ is selected from Aib, Ala, and Leu.
928. The agent of any one of the preceding Embodiments, wherein p21 is 0.
929. The agent of any one of the preceding Embodiments, wherein p22 is 1.
930. The agent of any one of the preceding Embodiments, wherein X²² comprises a hydrophobic side chain.
931. The agent of any one of the preceding Embodiments, wherein X²² is selected from Aib, Ala, and Leu.
932. The agent of any one of the preceding Embodiments, wherein p22 is 0.
933. The agent of any one of the preceding Embodiments, wherein p23 is 1.
934. The agent of any one of the preceding Embodiments, wherein X²³ comprises a hydrophobic side chain.
935. The agent of any one of the preceding Embodiments, wherein X²³ is selected from Aib, Ala, and Leu.
936. The agent of any one of the preceding Embodiments, wherein p23 is 0.
937. The agent of any one of the preceding Embodiments, wherein each amino acid residue is independently —N(R^{a1})-L^{a1}-C(R^{a2})(R^{a3})-L^{a2}-C(O)—.
938. The agent of Embodiment 937, wherein R^{a1} is —H.
939. The agent of any one of Embodiments 1-937, wherein R^{a1} are taken together with R^{a2} or R^{a3} and their intervening atom(s) to form an optionally substituted 3-10 (e.g., 3, 4, 5, 6, 7, 8, 9, or 10) membered ring having in addition to the intervening atom(s) 0-5 heteroatoms.
940. The agent of any one of Embodiments 1-937, wherein R^{a1} are taken together with R^{a2} or R^{a3} and their intervening atom(s) to form an optionally substituted 5-7 membered ring having in addition to the intervening atom(s) no heteroatoms.
941. The agent of any one of the preceding Embodiments, wherein L^{a1} is a bivalent C₁₋₆ aliphatic wherein one or more methylene units are optionally and independently replaced with —O—, —S—, —N(R′)—, —C(O)—, -Cy-, —C(O)N(R′)—, or —N(R′)C(O)O—.
942. The agent of any one of Embodiments 1-940, wherein L^{a1} is a covalent bond.
943. The agent of any one of the preceding Embodiments, wherein R^{a2} is -L^{a}-R′ wherein, L^{a} is a covalent bond or an optionally substituted bivalent C₁₋₆ aliphatic wherein one or more methylene units are optionally and independently replaced with —O—, —S—, —N(R′)—, —C(O)—, -Cy-, —C(O)N(R′)—, or —N(R′)C(O)O—.
944. The agent of any one of the preceding Embodiments, wherein R^{a3} is -L^{a}-R′ wherein, L^{a} is a covalent bond or an optionally substituted bivalent C₁₋₆ aliphatic wherein one or more methylene units are optionally and independently replaced with —O—, —S—, —N(R′)—, —C(O)—, -Cy-, —C(O)N(R′)—, or —N(R′)C(O)O—.
945. The agent of any one of Embodiments 1-943, wherein R^{a3} is —H.
946. The agent of any one of Embodiments 1-943, wherein R^{a3} is optionally substituted C₁₋₆ aliphatic.
947. The agent of any one of the preceding Embodiments, wherein L^{a2} is a bivalent C₁₋₆ aliphatic wherein one or more methylene units are optionally and independently replaced with —O—, —S—, —N(R′)—, —C(O)—, -Cy-, —C(O)N(R′)—, or —N(R′)C(O)O—.
948. The agent of any one of Embodiments 1-946, wherein L^{a2} is a covalent bond.
949. The agent of any one of the preceding Embodiments, wherein the peptide comprises a hydrocarbon staple.
950. The agent of any one of the preceding Embodiments, wherein the peptide comprises a non-hydrogen staple.
951. The agent of any one of the preceding Embodiments, wherein the peptide comprises a staple whose chain comprises —N(R′)— or —O—C(O)—N(R′)—.
952. The agent of any one of the preceding Embodiments, wherein the peptide has the structure of:

R^{N}—[X]ₚ-X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇—[X]_{p′}-R^{C},
or a salt thereof, wherein:
   each X is independently an amino acid residue;
   each p and p′ is independently 0-10;
   R^{N} is independently a peptide, an amino protecting group or R′-L^{RN}-;
   R^{C} is independently a peptide, a carboxyl protecting group, -L^{RC}-R′, —O-L^{RC}-R′ or —N(R′)-L^{RC}-R′;
   each of L^{RN} and L^{RC} is independently L;
   each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each -Cy- is independently an optionally substituted bivalent, 3-30 membered, monocyclic, bicyclic or polycyclic ring having 0-10 heteroatoms;
   each R′ is independently -L-R, —C(O)R, —CO₂R, or —SO₂R;
   each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
   two R groups are optionally and independently taken together to form a covalent bond, or:
   two or more R groups on the same atom are optionally and independently taken together with the atom to form an optionally substituted, 3-30 membered, monocyclic, bicyclic or polycyclic ring having, in addition to the atom, 0-10 heteroatoms; or
   two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.
953. The agent of any one of the preceding Embodiments, wherein p is 0.
954. The agent of any one of the preceding Embodiments, wherein p′ is 0.
955. The agent of any one of the preceding Embodiments, wherein R^{N} is —C(O)R.
956. The agent of any one of the preceding Embodiments, wherein R^{N} is Ac.
957. The agent of Embodiment 952, wherein R^{N} is AzAc (N₃—CH₂—C(O)—).
958. The agent of Embodiment 952, wherein R^{N} is 2PyPrpc

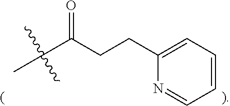 [SMILES: *C(=O)CCc1ccccn1]

959. The agent of Embodiment 952, wherein R^{N} is MeOPr (CH₃OCH₂CH₂C(O)—).
960. The agent of Embodiment 952, wherein R^{N} is MeSO₂ (—SO₂CH₃).
961. The agent of Embodiment 952, wherein R^{N} is mPEG2 (CH₃OCH₂CH₂OCH₂CH₂C(O)—).
962. The agent of Embodiment 952, wherein R^{N} is Nic

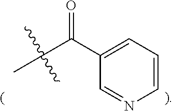 [SMILES: *C(=O)c1cccnc1]

963. The agent of Embodiment 952, wherein R^{N} is Oct (CH₃(CH₂)₆C(O)—).
964. The agent of Embodiment 952, wherein R^{N} is Pic

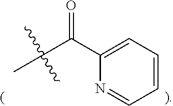 [SMILES: *C(=O)c1ccccn1]

965. The agent of any one of the preceding Embodiments, wherein R^{C} is —N(R′)₂.
966. The agent of any one of the preceding Embodiments, wherein R^{C} is —N(R)₂.
967. The agent of Embodiment 966, wherein R^{C} is —NH₂.
968. The agent of Embodiment 966, wherein R^{C} is —NHEt.
969. The agent of Embodiment 966, wherein R^{C} is —NHBn.
970. The agent of Embodiment 966, wherein R^{C} is —NHCyHe

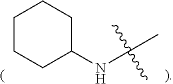 [SMILES: *NC1CCCCC1]

971. The agent of Embodiment 966, wherein R^{C} is —NHCyPr

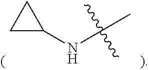

972. The agent of Embodiment 966, wherein R^{C} is −6AmHex, wherein one amino group of −6AmHex is bonded to the last —C(O)— of the peptide backbone (R^{C} is —NH—(CH₂)₆—NH₂).
973. The agent of Embodiment 966, wherein R^{C} is −6AZHex, wherein the amino group of −6AzHex is bonded to the last —C(O)— of the peptide backbone (R^{C} is —NH—(CH₂)₆—N₃).
974. The agent of Embodiment 966, wherein R^{C} is -Alaol, wherein the amino group of -Alaol is bonded to the last —C(O)— of the peptide backbone (R^{C} is

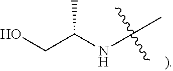 [SMILES: *N[C@@H](C)CO]

975. The agent of Embodiment 966, wherein R^{C} is -Leuol, wherein the amino group of -Leuol is bonded to the last —C(O)— of the peptide backbone (R^{C} is

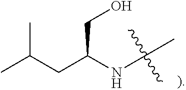 [SMILES: *N[C@H](CO)CC(C)C]

976. The agent of Embodiment 966, wherein R^{C} is -Pheol, wherein the amino group of -Pheol is bonded to the last —C(O)— of the peptide backbone (R^{C} is

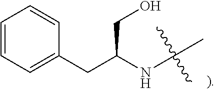 [SMILES: *N[C@H](CO)Cc1ccccc1]

977. The agent of Embodiment 966, wherein R^{C} is -Prool, wherein the amino group of -Prool is bonded to the last —C(O)— of the peptide backbone (R^{C} is

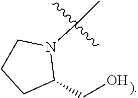 [SMILES: *N1CCC[C@H]1CO]

978. The agent of Embodiment 966, wherein R^{C} is -Throl, wherein the amino group of -Throl is bonded to the last —C(O)— of the peptide backbone (R^{C} is

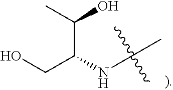 [SMILES: *N[C@H](CO)[C@@H](C)O]

979. The agent of any one of Embodiments 1-964, wherein R^{C} is —OH.
980. The agent of any one of the preceding Embodiments, wherein the peptide forms a structure that comprises a helix.
981. The agent of any one of the preceding Embodiments, wherein the peptide binds to beta-catenin.
982. The agent of any one of the preceding Embodiments, wherein the peptide binds to beta-catenin with a EC50 of no more than about 2000 nM, or no more than about 1500 nM, or no more than about 1000 nM, or no more than about 500 nM, or no more than about 300 nM, or no more than about 200 nM, or no more than about 100 nM, or no more than about 75 nM, or no more than about 50 nM, or no more than about 25 nM, or no more than about 10 nM as measured by fluorescence polarization.
983. The agent of any one of the preceding Embodiments, wherein the peptide can compete with TCF7, LEF1, TCF7L1, TCF7L2, Axin1, Axin2, or APC, or a fragment thereof, for beta-catenin binding.
984. The agent of any one of the preceding Embodiments, wherein the peptide binds to a polypeptide whose sequence is or comprising SEQ ID NO: 2, or a fragment thereof:

(SEQ ID NO: 2)
SVLFYAITTLHNLLLHQEGAKMAVRLAGGLQKMVALLNKTNVKFLAITTD
CLQILAYGNQESKLIILASGGPQALVNIMRTYTYEKLLWTTSRVLKVLSV
CSSNKPAIVEAGGMQALGLHLTDPSQRLVQNCLWTLRNLSDAATKQEGME
GLLGTLVQLLGSDDINVVTCAAGILSNLTCNNYKNKMMVCQVGGIEALVR
T.

985. The agent of any one of the preceding Embodiments, wherein the peptide binds to beta-catenin and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 1 at the indicated positions: A305, Y306, G307, N308, Q309, K312, R342, K345, V346, V349, Q375, R376, Q379, N380, L382, W383, R386, N387, D413, N415, V416, T418, and C419.
986. The agent of any one of the preceding Embodiments, wherein the peptide binds to beta-catenin and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 1 at the indicated positions: A305, Y306, G307, N308, Q309, K312, R342, K345, V346, V349, Q375, Q379, N380, L382, W383, R386, N387, D413, N415, V416, T418, and C419.
987. The agent of any one of the preceding Embodiments, wherein the peptide binds to beta-catenin and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 1 at the indicated positions: A305, Y306, G307, N308, Q309, K312, K345, V346, V349, Q379, N380, L382, W383, R386, N387, D413, N415, V416, T418, and C419.
988. The agent of any one of the preceding Embodiments, wherein the peptide binds to beta-catenin and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 1 at the indicated positions: G307, K312, K345, W383, N387, D413, and N415.
989. The agent of any one of the preceding Embodiments, wherein the agent interacts with G307 of beta-catenin or an amino acid residue corresponding thereto.
990. The agent of any one of the preceding Embodiments, wherein the agent interacts with K312 of beta-catenin or an amino acid residue corresponding thereto.
991. The agent of any one of the preceding Embodiments, wherein the agent interacts with K345 of beta-catenin or an amino acid residue corresponding thereto.
992. The agent of any one of the preceding Embodiments, wherein the agent interacts with W383 of beta-catenin or an amino acid residue corresponding thereto.
993. The agent of any one of the preceding Embodiments, wherein the agent interacts with N387 of beta-catenin or an amino acid residue corresponding thereto.
994. The agent of any one of the preceding Embodiments, wherein the agent interacts with D413 of beta-catenin or an amino acid residue corresponding thereto.
995. The agent of any one of the preceding Embodiments, wherein the agent interacts with N415 of beta-catenin or an amino acid residue corresponding thereto.
996. The agent of any one of the preceding Embodiments, wherein the agent binds to beta-catenin at a site that is not an axin binding site.
997. The agent of any one of the preceding Embodiments, wherein the agent binds to beta-catenin at a site that is not a Bcl9 binding site.
998. The agent of any one of the preceding Embodiments, wherein the agent binds to beta-catenin at a site that is not a TCF binding site.
999. The agent of any one of the preceding Embodiments, wherein the agent is the peptide.
1000. An agent having a structure selected from Table E3 or a salt thereof.
1001. An agent has the structure of

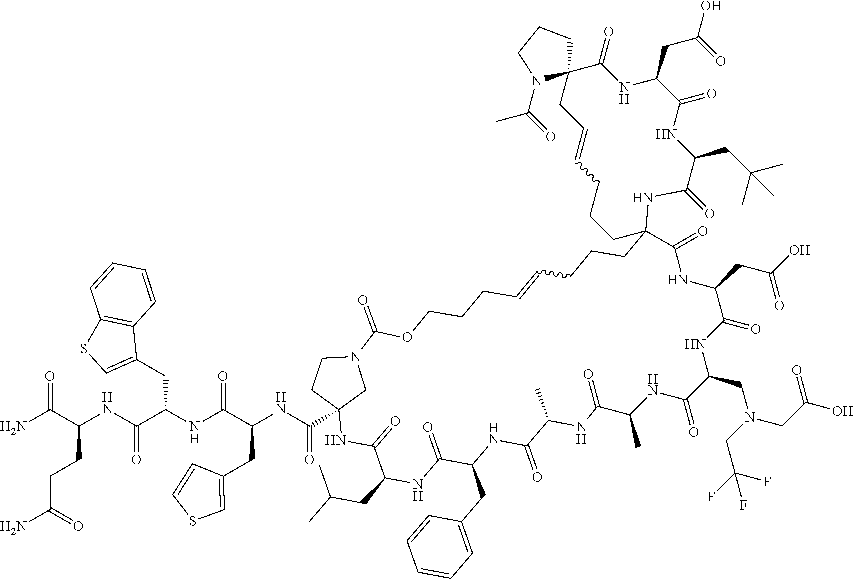 [SMILES: CC(=O)N1CCC[C@]12CC=CCCCC1(CCCC=CCCCOC(=O)N3CC[C@@](C(=O)N[C@@H](Cc4ccsc4)C(=O)N[C@@H](Cc4csc5ccccc45)C(=O)N[C@@H](CCC(N)=O)C(N)=O)(C3)NC(=O)[C@H](CC(C)C)NC(=O)[C@H](Cc3ccccc3)NC(=O)[C@H](C)NC(=O)[C@H](C)NC(=O)[C@H](CN(CC(=O)O)CC(F)(F)F)NC(=O)[C@H](CC(=O)O)NC1=O)NC(=O)[C@H](CC(C)(C)C)NC(=O)[C@H](CC(=O)O)NC2=O]

or a salt thereof.
1002. An agent has the structure of

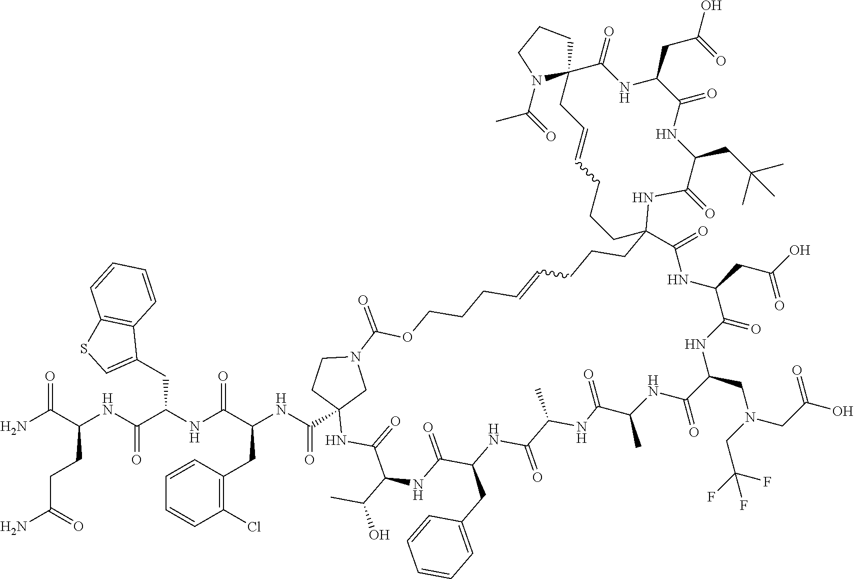 [SMILES: CC(=O)N1CCC[C@]12CC=CCCCC1(CCCC=CCCCOC(=O)N3CC[C@@](C(=O)N[C@@H](Cc4ccccc4Cl)C(=O)N[C@@H](Cc4csc5ccccc45)C(=O)N[C@@H](CCC(N)=O)C(N)=O)(C3)NC(=O)[C@H]([C@@H](C)O)NC(=O)[C@H](Cc3ccccc3)NC(=O)[C@H](C)NC(=O)[C@H](C)NC(=O)[C@H](CN(CC(=O)O)CC(F)(F)F)NC(=O)[C@H](CC(=O)O)NC1=O)NC(=O)[C@H](CC(C)(C)C)NC(=O)[C@H](CC(=O)O)NC2=O]

or a salt thereof.
1003. An agent has the structure of

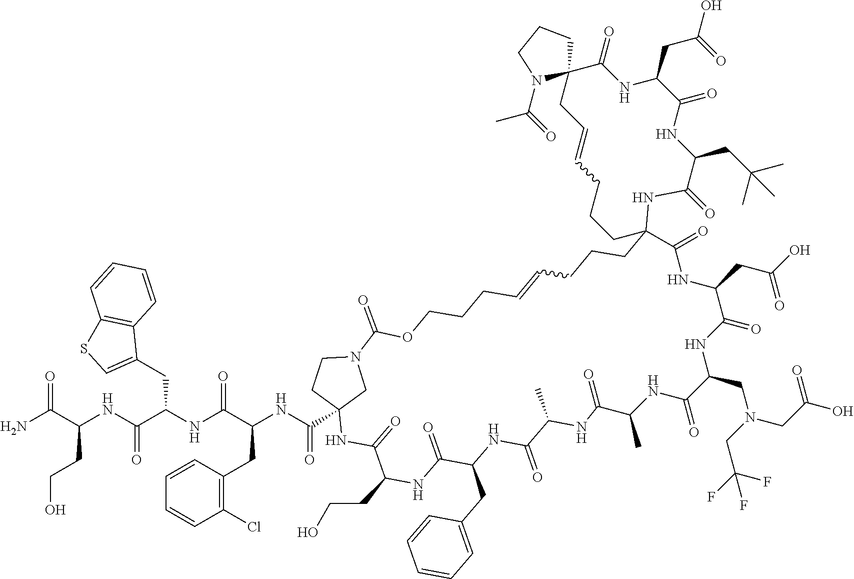 [SMILES: CC(=O)N1CCC[C@]12CC=CCCCC1(CCCC=CCCCOC(=O)N3CC[C@@](C(=O)N[C@@H](Cc4ccccc4Cl)C(=O)N[C@@H](Cc4csc5ccccc45)C(=O)N[C@@H](CCO)C(N)=O)(C3)NC(=O)[C@H](CCO)NC(=O)[C@H](Cc3ccccc3)NC(=O)[C@H](C)NC(=O)[C@H](C)NC(=O)[C@H](CN(CC(=O)O)CC(F)(F)F)NC(=O)[C@H](CC(=O)O)NC1=O)NC(=O)[C@H](CC(C)(C)C)NC(=O)[C@H](CC(=O)O)NC2=O]

or a salt thereof.
1004. The agent of any one of Embodiments 1001-1003, wherein a double bond of a staple bonded to the first amino acid from the N-terminus is E.
1005. The agent of any one of Embodiments 1001-1003, wherein a double bond of a staple bonded to the first amino acid from the N-terminus is Z.
1006. The agent of any one of Embodiments 1001-1005, wherein a double bond of a staple bonded to the 11th amino acid from the N-terminus is E.
1007. The agent of any one of Embodiments 1001-1005, wherein a double bond of a staple bonded to the 11^{th} amino acid from the N-terminus is Z.
1008. A compound having the structure of formula PA:

N(R^{PA})(R^{a1})-L^{a1}-C(R^{a2})(R^{a3})-L^{a2}-C(O)R^{C} PA
or a salt thereof, wherein:
   R^{PA} is —H or an amino protecting group;
   each of R^{a1} and R^{a3} is independently -L^{a}-R′;
   R^{a2} is -L^{aa}-C(O)R^{PS};
   each of L^{a}, L^{a1} and L^{a2} is independently L;
   —C(O)R^{PS} is optionally protected or activated —COOH;
   —C(O)R^{C} is optionally protected or activated —COOH;
   each L is independently a covalent bond, or an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each -Cy- is independently an optionally substituted bivalent, 3-30 membered, monocyclic, bicyclic or polycyclic ring having 0-10 heteroatoms;
   each R′ is independently —R, —C(O)R, —CO₂R, or —SO₂R; and
   each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
   two R groups are optionally and independently taken together to form a covalent bond, or:
   two or more R groups on the same atom are optionally and independently taken together with the atom to form an optionally substituted, 3-30 membered, monocyclic, bicyclic or polycyclic ring having, in addition to the atom, 0-10 heteroatoms; or
   two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.
1009. The compound of Embodiment 1008, wherein R^{a2} is -L^{aa}-C(O)R^{PS}, wherein L^{aa} is L and L^{aa} comprises —N(R′)— or -Cy-.
1010. The compound of any one of the preceding Embodiments, wherein L^{a1} is a covalent bond.
1011. The compound of any one of the preceding Embodiments, wherein L^{a2} is a covalent bond.
1012. The compound of any one of the preceding Embodiments, wherein L^{aa} is an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein at least one methylene unit is replaced with -Cy-.
1013. The compound of any one of the preceding Embodiments, wherein L^{aa} is -L^{am1}-Cy-L^{am2}-, wherein each of L^{am1}and L^{am2} is independently L^{am}, wherein each L^{am} is independently a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
1014. The compound of any one of the preceding Embodiments, wherein -L^{am2}- is bonded to —C(O)R^{PS}
1015. The compound of any one of the preceding Embodiments, wherein L^{am2} is a covalent bond.
1016. The compound of any one of the preceding Embodiments, wherein -Cy- is an optionally substituted 4-7 membered ring having 0-3 heteroatoms.
1017. The compound of any one of the preceding Embodiments, wherein -Cy- is an optionally substituted 6-10 membered aryl ring or is an optionally substituted 5-10 membered heteroaryl ring having 1-5 heteroatoms.
1018. The compound of any one of the preceding Embodiments, wherein -Cy- is an optionally substituted phenyl ring.
1019. The compound of any one of the preceding Embodiments, wherein -Cy- is optionally substituted

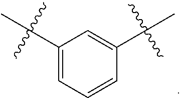 [SMILES: *c1cccc(*)c1]

1020. The compound of any one of the preceding Embodiments, wherein -Cy- is

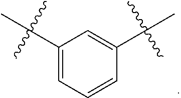 [SMILES: *c1cccc(*)c1]

1021. The compound of any one of Embodiments 1008-1015, wherein -Cy- is optionally substituted

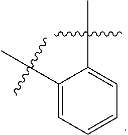 [SMILES: *c1ccccc1*]

1022. The compound of any one of Embodiments 1008-1015, wherein -Cy- is

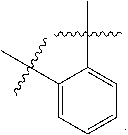 [SMILES: *c1ccccc1*]

1023. The compound of any one of Embodiments 1008-1015, wherein -Cy- is optionally substituted

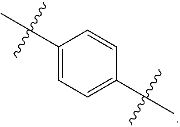 [SMILES: *c1ccc(*)cc1]

1024. The compound of any one of Embodiments 1008-1015, wherein -Cy- is

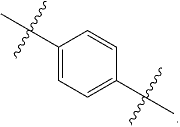 [SMILES: *c1ccc(*)cc1]

1025. The compound of any one of Embodiments 1008-1017, wherein -Cy- is an optionally substituted 5-10 membered heteroaryl ring having 1-5 heteroatoms.
1026. The compound of any one of Embodiments 1008-1017, wherein -Cy- is an optionally substituted 5-membered heteroaryl ring having 1-5 heteroatoms.
1027. The compound of any one of Embodiments 1008-1017, wherein -Cy- is optionally substituted

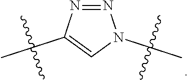 [SMILES: *c1cn(*)nn1]

1028. The compound of any one of Embodiments 1008-1017, wherein -Cy- is

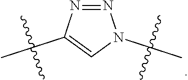 [SMILES: *c1cn(*)nn1]

1029. The compound of any one of the preceding Embodiments, wherein L^{aa} comprises —N(R′)—.
1030. The compound of Embodiment 1029, wherein L^{aa} is -L^{am1}-(NR′)-L^{am2}-, wherein each of L^{am1} and L^{am2} is independently L^{am}, wherein each L^{am} is independently a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
1031. The compound of any one of Embodiments 1029-1030, wherein R′ of the —N(R′)— is taken together with R^{a3} and their intervening atoms to form an optionally substituted 3-10 membered ring having 0-5 heteroatoms in addition to the intervening atoms.
1032. The compound of any one of Embodiments 1029-1031, wherein —N(R′)— is bonded to two carbon atoms which two carbon atoms do not form any double bonds with heteroatoms.
1033. The compound of any one of Embodiments 1029-1032, wherein -L^{am2}- is bonded to —C(O)R^{PS}.
1034. The compound of any one of Embodiments 1029-1033, wherein L^{am1} is optionally substituted C₁₋₄ alkylene.
1035. The compound of any one of Embodiments 1029-1033, wherein L^{am1} is optionally substituted —(CH₂)m-, wherein m is 1, 2, 3, or 4.
1036. The compound of any one of Embodiments 1029-1033, wherein L^{am1} is optionally substituted —CH₂—.
1037. The compound of any one of Embodiments 1029-1033, wherein L^{am1} is —CH₂—.
1038. The compound of any one of Embodiments 1029-1037, wherein L^{am2} is optionally substituted linear C₁₋₂ alkylene.
1039. The compound of any one of Embodiments 1029-1037, wherein L^{am2} is —[C(R′)₂]n, wherein n is 1 or 2.
1040. The compound of any one of Embodiments 1029-1037, wherein L^{am2} is —[CHR′]n, wherein n is 1 or 2.
1041. The compound of any one of Embodiments 1039-1040, wherein each R′ is independently —H or optionally substituted C₁₋₆ alkyl.
1042. The compound of any one of Embodiments 1029-1037, wherein L^{am2} is optionally substituted —CH₂—.
1043. The compound of any one of Embodiments 1029-1042, wherein L^{am2} is —CH₂—.
1044. The compound of any one of Embodiments 1029-1043, wherein L^{aa} comprises —N(R′)—, wherein R′ of the —N(R′)— is —R^{NR}, wherein R^{NR} is R.
1045. The compound of any one of Embodiments 1029-1043, wherein L^{aa} comprises —N(R′)—, wherein R′ of the —N(R′)— is —CH₂—R^{NR}, wherein R^{NR} is R.
1046. The compound of any one of Embodiments 1029-1043, wherein L^{aa} comprises —N(R′)—, wherein R′ of the —N(R′)— is —C(O)R^{NR}, wherein R^{NR} is R.
1047. The compound of any one of Embodiments 1029-1043, wherein L^{aa} comprises —N(R′)—, wherein R′ of the —N(R′)— is —SO₂R^{NR}, wherein R^{RN} is R.
1048. The compound of any one of Embodiments 1044-1047, wherein R^{NR} is optionally substituted C₁₋₆ aliphatic or heteroaliphatic having 1-4 heteroatoms.
1049. The compound of any one of Embodiments 1044-1048, wherein R^{NR} is C₁₋₇ alkyl or heteroalkyl having 1-4 heteroatoms, wherein the alkyl or heteroalkyl is optionally substituted with one or more groups independently selected from halogen, a C₅₋₆ aromatic ring having 0-4 heteroatoms, and an optionally substituted 3-10 membered cycloalkyl or heteroalkyl ring having 1-4 heteroatoms.
1050. The compound of any one of Embodiments 1044-1049, wherein R^{NR} is —CF₃.
1051. The compound of any one of Embodiments 1044-1048, wherein L^{am2} is or comprises —C(R′)₂— wherein the R′ group and R′ in —N(R′)— of L^{aa} are taken together with their intervening atom(s) to form an optionally substituted, 3-30 membered, monocyclic, bicyclic or polycyclic ring having, in addition to the intervening atom(s), 0-10 heteroatoms.
1052. The compound of any one of Embodiments 1008-1012, wherein L^{aa} is optionally substituted C₁₋₄ alkylene.
1053. The compound of Embodiment 1052, wherein L^{aa} is optionally substituted —CH₂—CH₂—.
1054. The compound of Embodiment 1052, wherein L^{aa} is optionally substituted —CH₂—.
1055. The compound of Embodiment 1008, having the structure of:

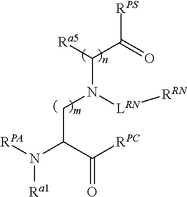

or a salt thereof, wherein:
   each of m and n is independently 1, 2, 3, or 4;
   L^{RN} is L;
   R^{RN} is R; and
   R^{a5} is R′.
1056. The compound of Embodiment 1055, wherein m is 1.
1057. The compound of any one of Embodiments 1055-1056, wherein L^{RN} is —CH₂—, —CO—, or —SO₂—.
1058. The compound of any one of Embodiments 1055-1056, wherein L^{RN} is —CH₂—.
1059. The compound of any one of Embodiments 1055-1058, wherein R^{NR} is C₁₋₇ alkyl or heteroalkyl having 1-4 heteroatoms, wherein the alkyl or heteroalkyl is optionally substituted with one or more groups independently selected from halogen, a C₅₋₆ aromatic ring having 0-4 heteroatoms, and an optionally substituted 3-10 membered cycloalkyl or heteroalkyl ring having 1-4 heteroatoms.
1060. The compound of any one of Embodiments 1055-1059, wherein one or more R^{a5} are independently —H.
1061. The compound of any one of Embodiments 1055-1060, wherein one or more R^{a5} are independently optionally substituted C₁₋₆ alkyl.
1062. The compound of any one of Embodiments 1055-1060, wherein -L^{RN}-R^{RN} is R, and is taken together with a R^{a5} and their intervening atom(s) to form an optionally substituted, 3-30 membered, monocyclic, bicyclic or polycyclic ring having, in addition to the intervening atom(s), 0-10 heteroatoms.
1063. The compound of Embodiment 1058, wherein R^{RN} is methyl.
1064. The compound of Embodiment 1058, wherein R^{RN} is —CF₃.
1065. The compound of any one of the preceding Embodiments, wherein R^{a1} is —H.
1066. The compound of any one of Embodiments 1008-1051, wherein R^{a1} is optionally substituted C₁₋₆ alkyl.
1067. The compound of any one of the preceding Embodiments, wherein —C(O)R^{PC} is a protected carboxylic acid group.
1068. The compound of any one of Embodiments 1008-1066, wherein —C(O)R^{PC} is an activated carboxylic acid group.
1069. The compound of any one of Embodiments 1008-1066, wherein —C(O)R^{PC} is —C(O)OR′.
1070. The compound of Embodiment 1069, wherein R′ is —H.
1071. The compound of Embodiment 1069, wherein R′ is pentafluorophenyl.
1072. The compound of Embodiment 1069, wherein R′ is

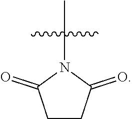 [SMILES: *N1C(=O)CCC1=O]

1073. The compound of any one of the preceding Embodiments, wherein —C(O)R^{PS} is —C(O)OR′.
1074. The compound of Embodiment 1073, wherein R′ is —H.
1075. The compound of Embodiment 1073, wherein R′ is optionally substituted C₁₋₆ aliphatic.
1076. The compound of Embodiment 1073, wherein R′ is t-butyl.
1077. The compound of Embodiment 1073, wherein R′ is benzyl.
1078. The compound of Embodiment 1073, wherein R′ is allyl.
1079. The compound of Embodiment 1008, wherein the compound has the structure of

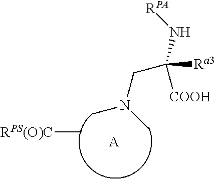

or a salt thereof, wherein Ring A is an optionally substituted 3-7 membered saturated, partially unsaturated or aromatic ring.
1080. The compound of Embodiment 1008, wherein the compound has the structure of

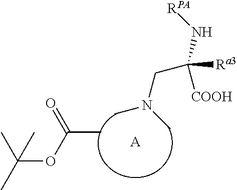

or a salt thereof, wherein Ring A is an optionally substituted 3-7 membered saturated, partially unsaturated or aromatic ring.
1081. The compound of any one of Embodiment 1079 or 1080, wherein —C(O)OtBu is bonded to a chiral carbon atom having a R configuration.
1082. The compound of any one of Embodiment 1079 or 1080, wherein —C(O)OtBu is bonded to a chiral carbon atom having a S configuration.
1083. The compound of Embodiment 1008, wherein the compound has the structure of

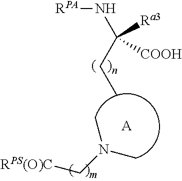

or a salt thereof, wherein:
   Ring A is an optionally substituted 3-10 membered ring;
   n is 0, 1, or 2; and
   m is 0, 1, 2, or 3.
1084. The compound of Embodiment 1008, wherein the compound has the structure of

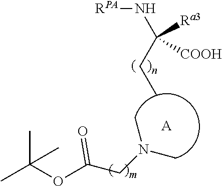

or a salt thereof, wherein:
   Ring A is an optionally substituted 3-10 membered ring;
   n is 0, 1, or 2; and
   m is 0, 1, 2, or 3.
1085. The compound of any one of Embodiments 1083-1084, wherein Ring A is an optionally substituted 4-10 membered ring.
1086. The compound of any one of Embodiments 1083-1085, wherein n is 1.
1087. The compound of any one of Embodiments 1083-1086, wherein Ring A is bonded to —(CH₂)ₙ— at a chiral carbon which is R.
1088. The compound of any one of Embodiments 1083-1086, wherein Ring A is bonded to —(CH₂)ₙ— at a chiral carbon which is S.
1089. The compound of Embodiment 1008, wherein the compound has the structure of

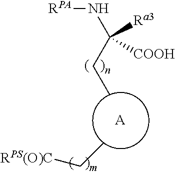

or a salt thereof, wherein:
   Ring A is an optionally substituted 3-10 membered ring;
   n is 0, 1, or 2; and
   m is 0, 1, 2, or 3.
1090. The compound of Embodiment 1008, wherein the compound has the structure of

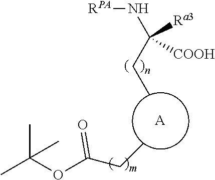

or a salt thereof, wherein:
   Ring A is an optionally substituted 3-10 membered ring;
   n is 0, 1, or 2; and
   m is 0, 1, 2, or 3.
1091. The compound of Embodiment 1008, wherein the compound has the structure of

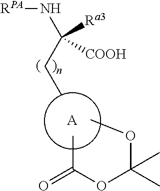

or a salt thereof, wherein:
   Ring A is an optionally substituted 3-10 membered ring; and
   n is 0, 1, or 2.
1092. The compound of any one of Embodiments 1083-1091, wherein n is 1.
1093. The compound of any one of Embodiments 1083-1092, wherein m is 0.
1094. The compound of any one of Embodiments 1083-1092, wherein m is 1, 2, or 3.
1095. The compound of any one of Embodiments 1083-1092, wherein m is 1.
1096. The compound of any one of Embodiments 1083-1095, wherein Ring A is or comprises an optionally substituted saturated monocyclic ring.
1097. The compound of any one of Embodiments 1083-1096, wherein Ring A is or comprises an optionally substituted partially unsaturated monocyclic ring.
1098. The compound of any one of Embodiments 1083-1097, wherein Ring A is or comprises an optionally substituted aromatic monocyclic ring.
1099. The compound of any one of Embodiments 1089-1095, wherein Ring A is optionally substituted phenyl.
1100. The compound of any one of Embodiments 1083-1095, wherein Ring A is optionally substituted 5−6 membered heteroaryl having 1-3 heteroatoms.
1101. The compound of any one of Embodiments 1083-1095, wherein Ring A is optionally substituted 5−6 membered heteroaryl having 1-3 heteroatoms, wherein at least one heteroatom is nitrogen.
1102. The compound of Embodiment 1101, wherein Ring A is an optionally substituted triazole ring.
1103. The compound of any one of Embodiments 1083-1095, wherein Ring A is an optionally substituted 8-10 membered bicyclic ring having 1−6 heteroatoms.
1104. The compound of any one of Embodiments 1083-1086, wherein Ring A is an optionally substituted 8-10 membered bicyclic aromatic ring having 1−6 heteroatoms, wherein each monocyclic unit is independently an optionally 5−6 membered aromatic ring having 0-3 heteroatoms.
1105. The compound of any one of Embodiments 1100-1104, wherein Ring A is bonded to —(CH₂)ₙ— at a carbon atom.
1106. The compound of any one of Embodiments 1100-1104, wherein Ring A is bonded to —(CH₂)ₙ— at a nitrogen atom.
1107. The compound of any one of the preceding Embodiments, wherein Ring A or -Cy- in L^{aa} is optionally substituted, and each substitute is independently selected from halogen, —R, —CF₃, —N(R)₂, —CN, and —OR, wherein each R is independently C₁₋₆ aliphatic optionally substituted with one or more —F.
1108. The compound of any one of the preceding Embodiments, wherein Ring A or -Cy- in L^{aa} is optionally substituted, and each substitute is independently selected from halogen, C₁₋₅ linear, branched or cyclic alkyl, —OR wherein R is C₁₋₄ linear, branched or cyclic alkyl, fluorinated alkyl, —N(R)₂ wherein each R is independently C₁₋₆ linear, branched or cyclic alkyl, or —CN.
1109. The compound of any one of the preceding Embodiments, wherein R^{a3} is —H or optionally substituted C₁₋₆ aliphatic.
1110. The compound of any one of the preceding Embodiments, wherein R^{a3} is —H.
1111. The compound of any one of Embodiments 1008-1109, wherein R^{a3} is methyl.
1112. A compound having the structure of:

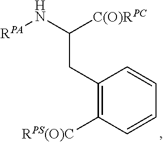

or a salt thereof, wherein:
   R^{PA} is —H or an amino protecting group;
   —C(O)R^{PS} is optionally protected or activated —COOH; and
   —C(O)R^{C} is optionally protected or activated —COOH.
1113. A compound having the structure of:

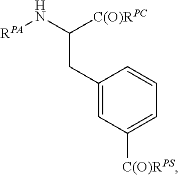

or a salt thereof, wherein:
   R^{PA} is —H or an amino protecting group;
   —C(O)R^{PS} is optionally protected or activated —COOH; and
   —C(O)R^{C} is optionally protected or activated —COOH.
1114. The compound of any one of the preceding Embodiments, wherein R^{PA} is an amino protecting group suitable for peptide synthesis.
1115. The compound of any one of the preceding Embodiments, wherein R^{PA} is —C(O)—O—R.
1116. The compound of Embodiment 1115, wherein R is optionally substituted

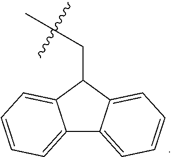 [SMILES: *CC1c2ccccc2-c2ccccc21]

1117. The compound of any one of the preceding Embodiments, wherein R^{PA} is -Fmoc.
1118. The compound of any one of the preceding Embodiments, wherein R^{PA} is —Cbz.
1119. The compound of any one of the preceding Embodiments, wherein R^{PA} is -Boc.
1120. The compound of any one of the preceding Embodiments, wherein R^{PS} is a protecting group orthogonal to R^{PA}.
1121. The compound of any one of the preceding Embodiments, wherein R^{PS} is a protecting group orthogonal to R^{PC}.
1122. The compound of any one of the preceding Embodiments, wherein R^{PS} is compatible with peptide synthesis.
1123. The compound of any one of the preceding Embodiments, wherein —C(O)R^{PS} is —C(O)OR′.
1124. The compound of Embodiment 1073, wherein R′ is —H.
1125. The compound of Embodiment 1073, wherein R′ is optionally substituted C₁₋₆ aliphatic.
1126. The compound of Embodiment 1073, wherein R′ is t-butyl.
1127. The compound of Embodiment 1073, wherein R′ is benzyl.
1128. The compound of Embodiment 1073, wherein R′ is allyl.
1129. The compound of any one of Embodiments 1008-1122, wherein —C(O)R^{PS} is —C(O)S-L-R′.
1130. The compound of Embodiment 1129, wherein L is optionally substituted —CH₂—.
1131. The compound of Embodiment 1129, wherein L is —CH₂—.
1132. The compound of any one of Embodiments 1129-1131, wherein R′ is optionally substituted phenyl.
1133. The compound of any one of Embodiments 1129-1131, wherein R′ is 2, 4, 6-trimethoxyphenyl.
1134. The compound of Embodiment 1129, wherein R^{PS} is —SH.
1135. The compound of any one of the preceding Embodiments, wherein —C(O)R^{PC} is a protected carboxylic acid group.
1136. The compound of any one of Embodiments 1008-1133, wherein —C(O)R^{PC} is an activated carboxylic acid group.
1137. The compound of any one of Embodiments 1008-1133, wherein —C(O)R^{C} is —C(O)OR′.
1138. The compound of Embodiment 1137, wherein R′ is —H.
1139. The compound of Embodiment 1137, wherein R′ is pentafluorophenyl.
1140. The compound of Embodiment 1137, wherein R′ is

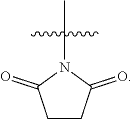 [SMILES: *N1C(=O)CCC1=O]

1141. The compound of any one of the preceding Embodiments, wherein each heteroatom is independently selected from oxygen, nitrogen, sulfur, phosphorus and silicon.
1142. The compound of any one of the preceding Embodiments, wherein each heteroatom is independently selected from oxygen, nitrogen, and sulfur.
1143. A compound, wherein the compound is

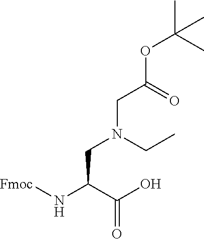

or a salt thereof.
1144. A compound, wherein the compound is

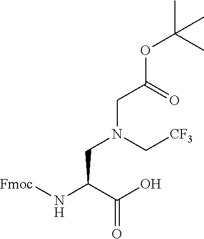

or a salt thereof.
1145. A compound, wherein the compound is

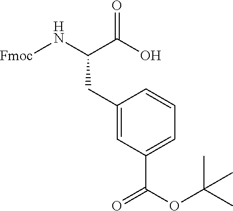

or a salt thereof.
1146. A compound, wherein the compound is

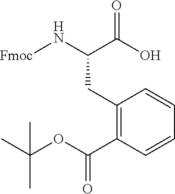

or a salt thereof.
1147. A compound, wherein the compound is

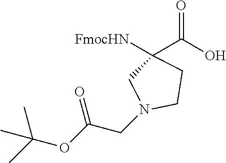

or a salt thereof.
1148. A compound, wherein the compound is

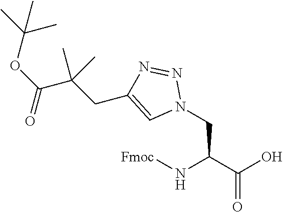

or a salt thereof.
1149. A compound, wherein the compound is

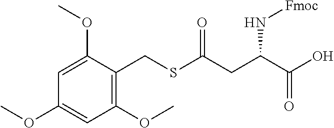

or a salt thereof.
1150. A compound, wherein the compound is

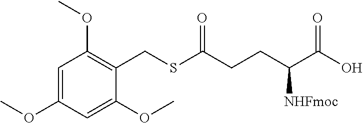

or a salt thereof.
1151. A compound, wherein the compound is

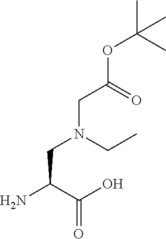 [SMILES: CCN(CC(=O)OC(C)(C)C)C[C@H](N)C(=O)O]

or a salt thereof.
1152. A compound, wherein the compound is

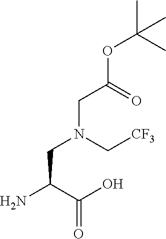 [SMILES: CC(C)(C)OC(=O)CN(C[C@H](N)C(=O)O)CC(F)(F)F]

or a salt thereof.
1153. A compound, wherein the compound is

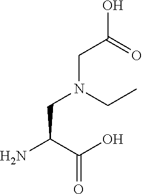

or a salt thereof.
1154. A compound, wherein the compound is

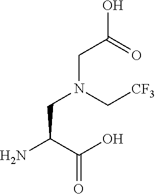 [SMILES: N[C@@H](CN(CC(=O)O)CC(F)(F)F)C(=O)O]

or a salt thereof.
1155. A compound, wherein the compound is

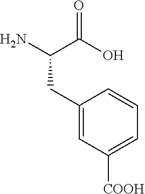 [SMILES: N[C@@H](Cc1cccc(C(=O)O)c1)C(=O)O]

or a salt thereof.
1156. A compound, wherein the compound is

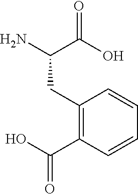 [SMILES: N[C@@H](Cc1ccccc1C(=O)O)C(=O)O]

or a salt thereof.
1157. A compound, wherein the compound is

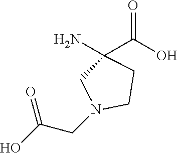 [SMILES: N[C@@]1(C(=O)O)CCN(CC(=O)O)C1]

or a salt thereof.
1158. A compound, wherein the compound is

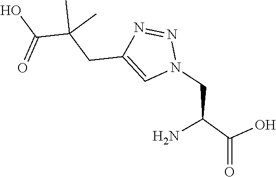 [SMILES: CC(C)(Cc1cn(C[C@H](N)C(=O)O)nn1)C(=O)O]

or a salt thereof.
1159. A compound, wherein the compound is

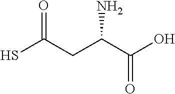

or a salt thereof.
1160. A compound, wherein the compound is

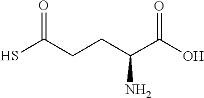

or a salt thereof.
1161. A compound, wherein the compound is

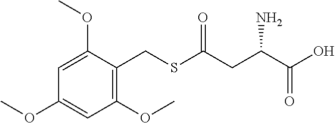 [SMILES: COc1cc(OC)c(CSC(=O)C[C@H](N)C(=O)O)c(OC)c1]

or a salt thereof.
1162. A compound, wherein the compound is

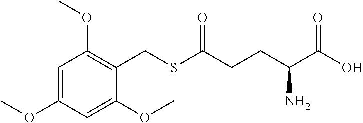 [SMILES: COc1cc(OC)c(CSC(=O)CC[C@H](N)C(=O)O)c(OC)c1]

or a salt thereof.
1163. The compound of any one of the preceding Embodiments, wherein the compound has a purity of at least about 90%, 91%, 92%, 93%, 94%, 95%, 96%, 97%, 98%, or 99%.
1164. A compound, comprising a residue of any one of the preceding Embodiments.
1165. A compound, comprising a residue of Table A-IV.
1166. A compound, comprising a residue having the structure of

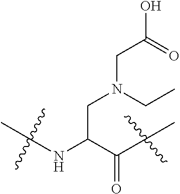 [SMILES: *NC(CN(CC)CC(=O)O)C(*)=O]

or a salt form thereof.
1167. A compound, comprising a residue having the structure of

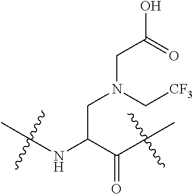 [SMILES: *NC(CN(CC(=O)O)CC(F)(F)F)C(*)=O]

or a salt form thereof.
1168. A compound, comprising a residue having the structure of

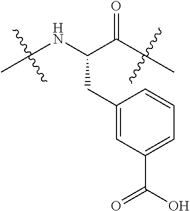 [SMILES: *N[C@@H](Cc1cccc(C(=O)O)c1)C(*)=O]

or a salt form thereof.
1169. A compound, comprising a residue having the structure of

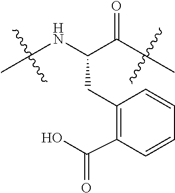 [SMILES: *N[C@@H](Cc1ccccc1C(=O)O)C(*)=O]

or a salt form thereof.
1170. A compound, comprising a residue having the structure of

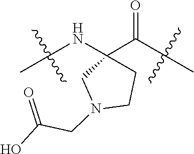 [SMILES: *N[C@@]1(C(*)=O)CCN(CC(=O)O)C1]

or a salt form thereof.
1171. A compound, comprising a residue having the structure of

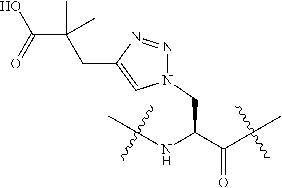 [SMILES: *N[C@@H](Cn1cc(CC(C)(C)C(=O)O)nn1)C(*)=O]

or a salt form thereof.
1172. A compound, comprising a residue having the structure of

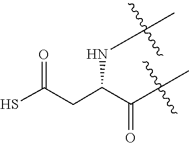 [SMILES: *N[C@@H](CC(=O)S)C(*)=O]

or a salt form thereof.
1173. A compound, comprising a residue having the structure of

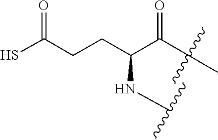 [SMILES: *N[C@@H](CCC(=O)S)C(*)=O]

or a salt form thereof.
1174. A compound, comprising a residue having the structure of

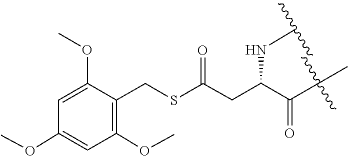 [SMILES: *N[C@@H](CC(=O)SCc1c(OC)cc(OC)cc1OC)C(*)=O]

or a salt form thereof.
1175. A compound, comprising a residue having the structure of

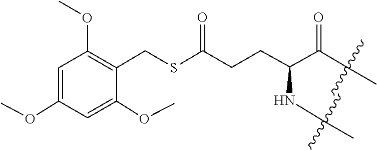 [SMILES: *N[C@@H](CCC(=O)SCc1c(OC)cc(OC)cc1OC)C(*)=O]

or a salt form thereof
1176. The compound of any one of Embodiments 1164-1175, wherein the compound is or comprise a peptide.
1177. The compound of any one of Embodiments 1164-1175, wherein the compound is an agent of any one of the preceding Embodiments.
1178. The compound of any one of Embodiments 1164-1175, wherein the compound is or comprise a stapled peptide.
1179. A method for preparing a compound of any one of Embodiments 1164-1178, comprising utilization of a compound of any one of the Embodiments 1008-1163.
1180. An agent, which agent comprises a residue of an amino acid of any one of the preceding Embodiments.
1181. The agent of any one of Embodiments 1-999, wherein the agent comprises a residue of an amino acid of any one of the preceding Embodiments.
1182. The agent of any one of the preceding Embodiments, wherein each olefin double bond in a staple is independently and optionally converted into a single bond.
1183. The agent of any one of the preceding Embodiments, wherein each olefin double bond in a staple is converted into a single bond.
1184. The agent of any one of the preceding Embodiments, wherein each olefin double bond is converted into a single bond.
1185. The agent of any one of the preceding Embodiments, wherein each olefin double bond is independently and optionally converted into —CHR′—CHR′—, wherein each R is independently —H, —R, —OR, —OH, —N(R)₂, or —SR.
1186. The agent of any one of the preceding Embodiments, wherein each olefin double bond is converted into —CHR′—CHR′—, wherein each R is independently —H, —R, —OR, —OH, —N(R)₂, or —SR.
1187. The agent of any one of the preceding Embodiments, wherein each olefin double bond is independently and optionally converted into optionally substituted —CH₂—CH₂—.
1188. The agent of any one of the preceding Embodiments, wherein each olefin double bond is converted into —CH₂—CH₂—.
1189. A pharmaceutical composition, comprising or delivering an agent or amino acid of any one of the preceding Embodiments, and a pharmaceutically acceptable carrier.
1190. A composition selected from Table E3.
1191. A pharmaceutical composition, comprising or delivering one or more or all peptide agents in a composition selected from Table E3 and a pharmaceutically acceptable carrier.
1192. A method for preparing an agent of any one of the preceding Embodiments, comprising incorporating a residue of an amino acid of any one of the preceding Embodiments.
1193. A method for modulating beta-catenin interaction with a partner in a system, comprising contacting beta-catenin with an agent or composition of any one of the preceding Embodiments.
1194. A method for modulating beta-catenin interaction with a partner in a system, comprising administering or delivering to the system an agent or composition of any one of the preceding Embodiments.
1195. The method of nay one of Embodiments 1193-1194, wherein the partner is TCF7, LEF1, TCF7L1, TCF7L2, Axin1, Axin2, or APC.
1196. A method for modulating a TCF-beta-catenin interaction in a system, comprising contacting beta-catenin with an agent or composition of any one of the preceding Embodiments.
1197. A method for inhibiting beta-catenin dependent cell proliferation, comprising administering or delivering to the system an agent or composition of any one of the preceding Embodiments.
1198. A method for modulating a TCF-beta-catenin interaction in a system, comprising administering or delivering to the system an agent or composition of any one of the preceding Embodiments.
1199. The method of any one of Embodiments 1193-1198, wherein a system is an in vitro system.
1200. The method of any one of Embodiments 1193-1198, wherein a system is or comprises a cell, tissue or organ.
1201. The method of any one of Embodiments 1193-1198, wherein a system is a subject.
1202. A method for treating or preventing a condition, disorder or disease associated with beta-catenin in a subject, comprising administering or delivering to the subject an effective amount of an agent or composition of any one of the preceding Embodiments.
1203. A method for treating cancer in a subject, comprising administering or delivering to the subject an effective amount of an agent or composition of any one of the preceding Embodiments.
1204. A method for treating or preventing a condition, disorder or disease associated with beta-catenin interaction with a partner in a subject, comprising administering or delivering to the subject an effective amount of an agent or composition of any one of the preceding Embodiments.
1205. The method of Embodiment 1204, wherein the partner is TCF7, LEF1, TCF7L1, TCF7L2, Axin1, Axin2, or APC.
1206. A method for treating or preventing a condition, disorder or disease associated with TCF-beta-catenin interaction in a subject, comprising administering or delivering to the subject an effective amount of an agent or composition of any one of the preceding Embodiments.
1207. The method of any one of the preceding Embodiments, wherein the condition, disorder or disease is melanoma.
1208. The method of any one of the preceding Embodiments, comprising administering or deliver to a subject a second therapeutic agent.
1209. The method of any one of the preceding Embodiments, comprising administering or deliver to a subject a second therapy.
1210. The method of Embodiment 1208, wherein a second therapeutic agent or therapy is administered prior to an agent of any one of the preceding Embodiments.
1211. The method of Embodiment 1208, wherein a second therapeutic agent or therapy is administered about or no more than about 1, 2, 3, 4, 5, 6, or 7 days, or 1, 2, 3, or weeks, or 1, 2, 3, 4, 5, or 6 months, prior to an agent of any one of the preceding Embodiments.
1212. The method of Embodiment 1208, wherein a second therapeutic agent or therapy is administered concurrently with an agent of any one of the preceding Embodiments.
1213. The method of Embodiment 1208, wherein a second therapeutic agent or therapy is administered subsequently to an agent of any one of the preceding Embodiments.
1214. The method of Embodiment 1208, wherein a second therapeutic agent or therapy is administered about or no more than about 1, 2, 3, 4, 5, 6, or 7 days, or 1, 2, 3, or weeks, or 1, 2, 3, 4, 5, or 6 months, subsequently to an agent of any one of the preceding Embodiments.
1215. The method of any one of the preceding Embodiments, wherein a subject is exposed to a second therapeutic agent or therapy and an agent of any one of the preceding Embodiments.
1216. The method of any one of the preceding Embodiments, wherein a subject is exposed to a therapeutic effect of a second therapeutic agent or therapy and a therapeutic effect of an agent of any one of the preceding Embodiments.
1217. The method of any one of the preceding Embodiments, wherein a second therapeutic agent is or comprises a chemotherapy agent.
1218. The method of any one of the preceding Embodiments, wherein a second therapeutic agent is or comprises a hormone therapy agent.
1219. The method of any one of the preceding Embodiments, wherein a second therapeutic agent is or comprises an immunotherapy agent.
1220. The method of any one of the preceding Embodiments, wherein a second therapeutic agent is or comprises a checkpoint inhibitor.
1221. The method of any one of the preceding Embodiments, wherein a second therapeutic agent is or comprises an antibody.
1222. The method of any one of the preceding Embodiments, wherein a second therapeutic agent is or comprises a CTLA-4, PD-1 or PD-L1 inhibitor.
1223. The method of any one of the preceding Embodiments, wherein a second therapeutic agent is or comprises a cell.
1224. The method of any one of the preceding Embodiments, wherein the second therapeutic agent reduces one or more side effects of an agent or composition of any one of the preceding Embodiments.
1225. The method of any one of the preceding Embodiments, wherein the agent or composition reduces one or more side effects of a second therapeutic agent.
1226. The method of any one of the preceding Embodiments, wherein a second therapy is or comprises surgery.
1227. The method of any one of the preceding Embodiments, wherein a second therapy is or comprises chemotherapy.
1228. The method of any one of the preceding Embodiments, wherein a second therapy is or comprises radiotherapy.
1229. The method of any one of the preceding Embodiments, wherein a second therapy is or comprises hormone therapy.
1230. The method of any one of the preceding Embodiments, wherein a second therapy is or comprises stem cell or bone marrow transplant.
1231. The method of any one of the preceding Embodiments, wherein a second therapy is or comprises immunotherapy.
1232. The method of any one of the preceding Embodiments, wherein a second therapy is or comprises T-cell therapy.
1233. The method of any one of the preceding Embodiments, wherein a second therapy is or comprises CAR T-cell therapy.
1234. The method of any one of the preceding Embodiments, wherein a second therapy is or comprises administering to the subject a population of immune cells.
1235. The method of any one of the preceding Embodiments, wherein the agent or composition reduces one or more side effects of a second therapy.
1236. The method of any one of the preceding Embodiments, wherein unit dose of a second therapy or therapeutic agent is reduced compared to when it is administered alone.
1237. The method of any one of the preceding Embodiments, wherein total dose of a second therapy or therapeutic agent is reduced compared to when it is administered alone.
1238. The method of any one of the preceding Embodiments, wherein unit dose of an agent or composition of any one of the preceding Embodiments is reduced compared to when it is administered alone.
1239. The method of any one of the preceding Embodiments, wherein total dose of an agent or composition of any one of the preceding Embodiments is reduced compared to when it is administered alone.
1240. The method of any one of the preceding Embodiments, wherein the combination therapy provides higher efficacy than when an agent or composition is administered or delivered alone.
1241. The method of any one of the preceding Embodiments, wherein the combination therapy provides higher efficacy than when a second therapeutic agent or therapy is administered or delivered alone.

### EXEMPLIFICATION

Those skilled in the art appreciate that various technologies are available for manufacturing and assessing provided agents including various peptides such as stapled peptides in accordance with the present disclosure, for example, many technologies for preparing small molecules and peptides can be utilized to prepare provided agents, and various assays are available for assessing properties and/or activities of provided agents. Described below are certain such useful technologies.

### Example 1. Peptide Synthesis

Among other things, peptides can be prepared using various peptide synthesis technologies in accordance with the present disclosure. In many embodiments, peptides were prepared using Fmoc-based synthesis, often on suitable solid phase. For various stapled peptides, amino acid residues were stapled through suitable chemistry, e.g., olefin metathesis for amino acids that comprise olefin groups. Those skilled in the art appreciates that other suitable technologies may also be utilized for stapling in accordance with the present disclosure, e.g., those described in WO/2019/051327, WO/2020/041270, etc., the peptide staples and technologies for preparing peptides are incorporated herein by reference.

For example, in some embodiments, peptides were synthesized on a Liberty Blue peptide synthesizer with 1 M DIC in DMF and 1 M Oxyma in DMF using standard Liberty Blue conditions on either Rink Protide amide resin (primary carboxamides), ethyl indole AM resin (ethyl amides), or amino alcohol 2-chlorotrityl resin (amino alcohols). Single coupling was used for all amino acids, save for residues following a stapling amino acid, which were double coupled. Deprotection was performed on the N-terminal residue, and capping, e.g., acetate capping, was performed by treating the resin with a suitable capping agent, e.g., 5% acetic anhydride, 2.5% diisopropylethylamine and 92.5% NMP for acetate capping, at room temperature for 30 min. In some embodiments, methanesulfonate capping was performed with 10 equivalents of methanesulfonyl chloride and 30 equivalents of diisopropylethylamine in dichloroethane. Non-acetate amide caps were generally performed on the Liberty Blue with standard coupling conditions. Olefin metathesis was performed by treating peptides with suitable metathesis catalysts under suitable conditions, in some embodiments, four cycles of 30 mol % Grubbs' first generation catalyst (CAS 172222-30-9) in dichloroethane at 40° C. for 2 h.

Side chain functionalization of Dap, Dab and Lys residues was performed by incorporating (ivDde)-protected amino acids, and after olefin metathesis, deprotection with two cycles of 5% hydrazine in DMF at 40° C. for 30 min. Side chain attachment was performed by coupling a carboxylic acid with HATU in DMF and diisopropylethylamine at 40° C. Side chain functionalization of substituted asparagine residues was performed by incorporating an Asp(2-phenylisopropyl ester) residue, and after olefin metathesis, deprotection with 5% trifluoroacetic acid in dichloromethane for 10 min. Side chain attachment was performed by coupling an amine with HATU and DMF and diisopropylethylamine at 40° C.

Peptide cleavage was performed by treating resin with 95% trifluoroacetic acid and 5% triisopropylsilane for 1 h, and precipitation of the crude peptide in diethyl ether. Purification was performed by preparative HPLC with MS detection and a Waters XSelect CSH C18 column using water with 0.1% formic acid and acetonitrile with 0.1% formic acid. Typically, if isomers were identified and separated by HPLC purification they were isolated and tested separately, otherwise peptides were isolated (often based on HPLC peaks) and tested as combinations (all peptides within a single HPLC peak were tested together in a single composition).

Amino acids suitable for synthesis are commercially available or can be prepared in accordance with the present disclosure. Certain preparations are presented below as examples.

### Example 1-a. Synthesis of (S)-2-((((9H-fluoren-9-yl)methoxy)carbonyl)amino)-3-(2-(tert-butoxycarbonyl)phenyl)propanoic acid

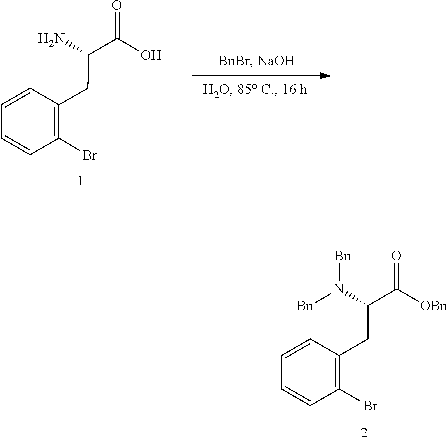 [SMILES: N[C@@H](Cc1ccccc1Br)C(=O)O ; O=C(OCc1ccccc1)[C@H](Cc1ccccc1Br)N(Cc1ccccc1)Cc1ccccc1]

To a solution of compound 1 in H₂O (250 mL) was added NaOH (84.0 g, 2.10 mol, 5.13 eq) and BnBr (328 g, 1.92 mol, 228 mL, 4.69 eq). The mixture was stirred at 85° C. for 16 hrs. LC-MS (EW24702-4-P1A) showed the compound 1 was consumed completely, and desired mass was detected (Rₜ=1.211 min). The mixture was cooled to 40° C. and the aqueous layer was removed. EtOAc (600 mL) and a mixture of methanol and water (1:2, 300 mL) were added. The mixture was washed with H₂O (300 mL). The organic layer was dried over Na₂SO₄, filtered and concentrated in vacuum. The residue was purified by column chromatography (SiO₂, Petroleum ether/Ethyl acetate=1/0, Petroleum ether:Ethyl acetate=10:1, R_{f}=0.7). Compound 2 (206 g, 400 mmol, 97.7% yield) was obtained as yellow oil. LCMS: Rₜ=1.211 min, m/z=514.3.1 (M+1)⁺. ¹HNMR (CDCl₃, 400 MHz) δ: 7.61 (dd, J₁=1.2 Hz, J₂=7.6 Hz, 1H), 7.92-7.87 (m, 5H), 7.77-7.66 (m, 11H), 7.64-7.59 (m, 2H), 5.81-5.63 (m, 2H), 4.48 (d, J=14 Hz, 2H), 4.41 (t, J=7.6 Hz, 1H), 4.07 (d, J=14 Hz, 2H), 3.74-3.69 (m, 2H).

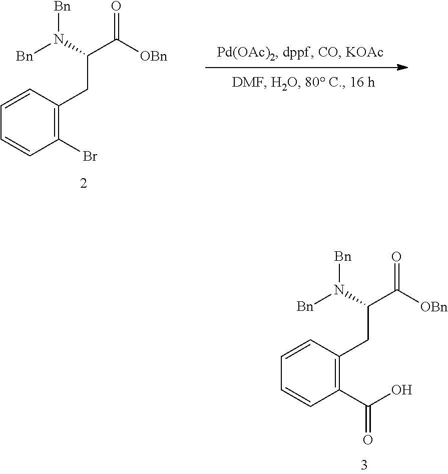 [SMILES: O=C(OCc1ccccc1)[C@H](Cc1ccccc1Br)N(Cc1ccccc1)Cc1ccccc1 ; O=C(O)c1ccccc1C[C@@H](C(=O)OCc1ccccc1)N(Cc1ccccc1)Cc1ccccc1]

A mixture of compound 2 (50.0 g, 97.1 mmol, 1.00 eq), Pd(OAc)₂ (1.09 g, 4.86 mmol, 0.05 eq), DPPF (5.39 g, 9.72 mmol, 0.1 eq) and KOAc (14.5 g, 147 mmol, 1.52 eq) in DMF (400 mL) and H₂O (100 mL) was degassed and purged with CO for 3 times, and then the mixture was stirred at 80° C. for 16 hrs under CO (50 psi) atmosphere. LC-MS (EW24702-5-P1A) showed the compound 2 wasn't consumed completely, and desired mass was detected (Rₜ=1.068 min). The reaction mixture was filtered. The filtrated was extracted with EtOAc (150 mL*3). The combined organic layers were washed with saturated brine (150 mL*3), dried over Na₂SO₄, filtered and concentrated under reduced pressure to give a residue. The crude product was purified by reversed-phase HPLC (FA condition). Compound 3 was obtained as yellow oil. LCMS: Rₜ=1.068 min, m/z=480.3 (M+1)⁺. ¹HNMR (CDCl3, 400 MHz) δ: 7.61 (dd, J₁=1.2 Hz, J₂=7.6 Hz, 1H), 7.44-7.39 (m, 8H), 7.20-7.10 (m, 11H), 5.29 (d, J=12.4 Hz 1H), 5.13 (d, J=12.4 Hz, 1H), 3.96 (d, J=13.6 Hz, 2H), 3.90-3.86 (m, 1H), 3.60-3.57 (m, 2H), 3.48-3.43 (m, 2H).

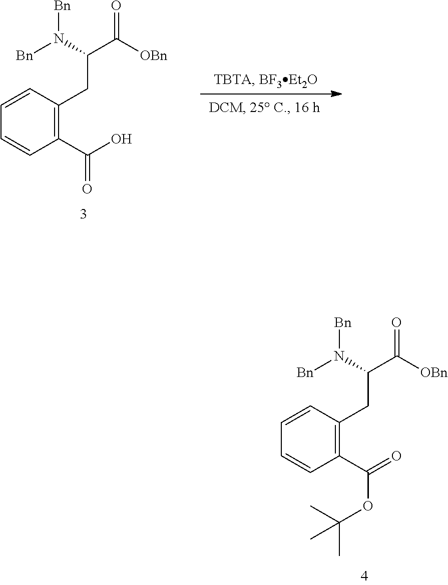 [SMILES: O=C(O)c1ccccc1C[C@@H](C(=O)OCc1ccccc1)N(Cc1ccccc1)Cc1ccccc1 ; CC(C)(C)OC(=O)c1ccccc1C[C@@H](C(=O)OCc1ccccc1)N(Cc1ccccc1)Cc1ccccc1]

To a solution of compound 3 (23.0 g, 47.9 mmol, 1.00 eq) in THF (300 mL) was added TBTA (52.4 g, 239 mmol, 42.9 mL, 5.00 eq), BF₃·Et₂O (680 mg, 4.80 mmol, 591 uL, 0.1 eq), the mixture was stirred at 25° C. for 3 hrs. LC-MS (EW24702-8-P1A) showed the compound 3 was consumed completely, and desired mass was detected (R_{f}=1.279 min). The reaction mixture was quenched by addition citric acid 100 mL and extracted with EtOAc (200 mL*3). The combined organic layers were washed with saturated brine (200 mL*3), dried over Na₂SO₄, filtered and concentrated under reduced pressure to give a residue. The residue was purified by column chromatography (SiO₂, Petroleum ether/Ethyl acetate=1/0 to 10/1, Petroleum ether:Ethyl acetate=10/1, R_{f}=0.4). Compound 4 (25.0 g, 46.6 mmol, 97.3% yield) was obtained as colorless oil. LCMS: EW24702-8-P1A, Rₜ=1.279 min, m/z=536.5 (M+1)⁺. ¹HNMR (CDCl₃, 400 MHz) δ: 7.85 (dd, J₁=0.8 Hz, J₂=1.2 Hz, 1H), 7.42-7.29 (m, 7H), 7.17-7.14 (m, 7H), 7.05-7.03 (m, 4H), 5.28 (d, J=12.4 Hz, 1H), 5.16 (d, J=12.4 Hz, 1H), 3.96 (d, J=13.2 Hz, 2H), 3.85-3.81 (m, 1H), 3.61-3.56 (m, 1H), 3.51 (d, J=14 Hz, 2H), 3.34-3.28 (m, 1H), 1.35 (s, 9H). Chiral SFC: Column: Chiralcel OJ-3 50×4.6 mm I.D., 3 um. Mobile phase: Phase A for CO₂, and Phase B for MeOH (0.05% DEA); Gradient elution: MeOH (0.05% DEA) in CO₂ from 5% to 40%; Flow rate: 3 mL/min; Detector: PDA; Column Temp: 35° C.; Back Pressure: 100 Bar; Chiral purity: 100%.

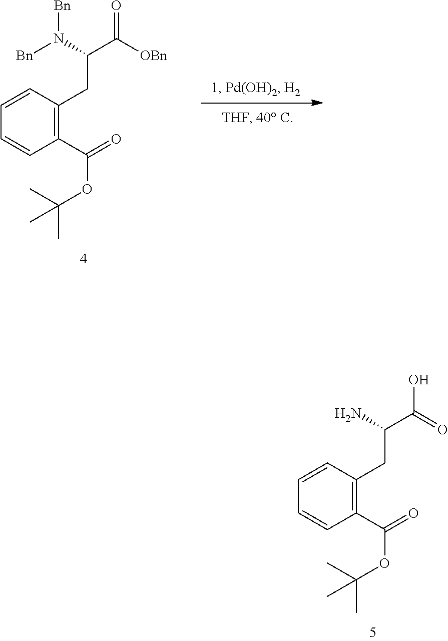 [SMILES: CC(C)(C)OC(=O)c1ccccc1C[C@@H](C(=O)OCc1ccccc1)N(Cc1ccccc1)Cc1ccccc1 ; CC(C)(C)OC(=O)c1ccccc1C[C@H](N)C(=O)O]

Two batches were combined together. A mixture of compound 4 (25.0 g, 46.6 mmol, 1.00 eq) and Pd(OH)₂ (3.00 g, 4.27 mmol, 20.0% purity, 9.15e⁻² eq) in THF (750 mL) was degassed and purged with H₂ for 3 times. The mixture was stirred at 40° C. for 16 hrs under H₂ atmosphere (50 psi). LC-MS (EW24072-13-P1C) showed the compound 4 was consumed, desired mass was detected (Rₜ=0.740 min). The mixture was filtered, and the filtrated was used to the next reaction directly. LCMS Rₜ=0.740 min, m/z=210.1 (M−55)⁺.

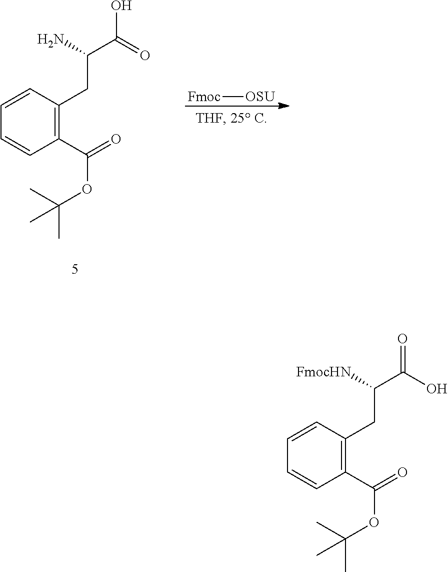

A mixture of compound 5 (dissolved in THF), FMOC-OSU (11.1 g, 33.1 mmol, 0.8 eq) in THF (50.0 mL) was degassed and purged with N₂ for 3 times, and then the mixture was stirred at 25° C. for 12 hrs. LC-MS (EW24702-14-P1A) showed the compound 5 was consumed completely, and desired mass was detected (Rₜ=0.998 min). The mixture was filtered, and filtrated was concentrated under vacuum. Three batches were combined together. The mixture was purified with reversed-phase HPLC (TFA condition). 2COOHF (13.2 g, 19.1 mmol, 46.2% yield, 98.3% purity) was obtained as yellow solid. LCMS Rₜ=0.983 min, m/z=510.2 (M+23)⁺; HPLC Rₜ=3.49 min, purity: 98.3%. ¹HNMR (DMSO, 400 MHz) δ: 12.7 (s, 1H), 7.87 (d, J=7.6 Hz, 2H), 7.76-7.68 (m, 2H), 7.63-7.59 (m, 2H), 7.42-7.40 (m, 2H), 7.38-7.34 (m, 2H), 7.32-7.30 (m, 2H), 7.29-7.26 (m, 2H), 4.31-4.29 (m, 1H), 4.21-4.15 (m, 2H), 4.13-4.10 (m, 1H), 3.54 (d, J=5.2 Hz, 1H), 3.00-2.96 (m, 1H), 1.54 (s, 9H). Chiral SFC: Chiral purity: 100%; Column: Chiralcel OJ-3 50×4.6 mm I.D., 3 um; Mobile phase: Phase A for CO₂, and Phase B for MeOH (0.05% DEA); Gradient elution: MeOH (0.05% DEA) in CO2 from 5% to 40%; Flow rate: 3 mL/min; Detector: PDA; Column Temp: 35° C.; Back Pressure: 100Bar): Chiral purity: 100%.

### Example 1-b. Synthesis of tert-butyl (S)-3-(2-((((9H-fluoren-9-yl)methoxy)carbonyl)amino)-3-(benzyloxy)-3-oxopropyl)benzoate

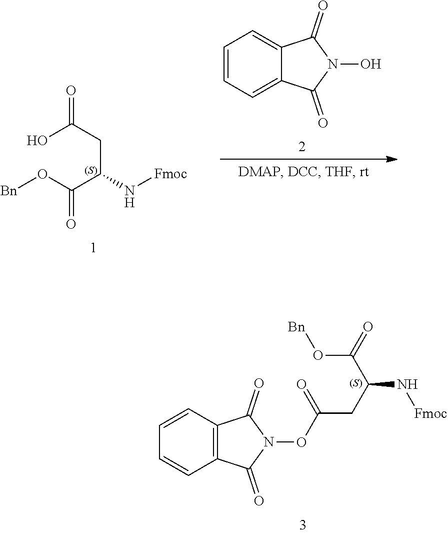

A mixture of compound 1 (90.0 g, 202 mmol, 1.00 eq), compound 2 (49.4 g, 303 mmol, 1.50 eq), DCC (50.0 g, 242 mmol, 49.0 mL, 1.20 eq), DMAP (1.23 g, 10.1 mmol, 0.0500 eq) in THF (100 mL) was degassed and purged with N₂ for 3 times, and then the mixture was stirred at 20° C. for 12 hrs under N₂ atmosphere. LC-MS (EW23957-10-P1B) showed the compound 1 was consumed completely, and desired mass was detected (Rₜ=1.046 min). The mixture was filtered, and the filtrate was concentrated in vacuo. The crude product was purified by reversed-phase HPLC (0.1% FA condition). Compound 3 (68.0 g, 115 mmol, 56.9% yield) was obtained as a white solid. LCMS Rₜ=1.046 min, m/z=613.1 (M+23)⁺. ¹HNMR (DMSO, 400 MHz) δ: 8.11 (d, J=8.4 Hz, 1H), 7.98-7.96 (m, 4H), 7.89 (d, J=7.6 Hz, 2H), 7.69 (d, J=7.6 Hz, 2H), 7.42-7.28 (m, 10H), 5.16 (d, J=2.0 Hz, 2H), 4.64-4.62 (m, 1H), 4.33-4.31 (m, 2H), 4.24-4.21 (m, 1H), 3.39-3.37 (m, 1H), 3.25-3.19 (m, 1H).

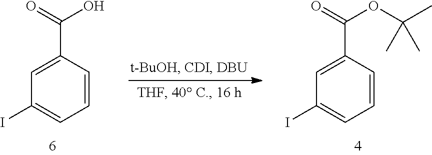

To a solution of compound 6 (100 g, 403 mmol, 1.00 eq) in THF (500 mL) was added CDI (71.9 g, 443 mmol, 1.10 eq) and the mixture was stirred for 0.5 h. 2-methylpropan-2-ol (387 g, 5.23 mol, 500 mL, 12.9 eq) and DBU (67.5 g, 443 mmol, 66.8 mL, 1.10 eq) were subsequently added to the reaction. The mixture was stirred at 40° C. for 11.5 hrs. TLC (Petroleum ether:Ethyl acetate=5/1) showed the compound 6 was consumed completely (R_{f}=0.15), and two main spots were observed (Rₜ=0.90, 0). H₂O (500 mL) was added to the reaction. The mixture was extracted with EtOAc (500 mL*3). The combined organic layers were washed with saturated brine (500 mL*3), dried over Na₂SO₄, filtered and concentrated under reduced pressure to give a residue. The residue was purified by column chromatography (SiO₂, Petroleum ether/Ethyl acetate=0/1, R_{f}=0.90). Compound 4 (113 g, 371 mmol, 92.1% yield) was obtained as alight yellow oil. ¹HNMR (DMSO, 400 MHz) δ: 8.16 (t, J=1.6 Hz, 1H), 7.97 (m, 1H), 7.89 (m, 1H), 7.30 (t, J=8.0 Hz, 1H), 1.53 (s, 9H).

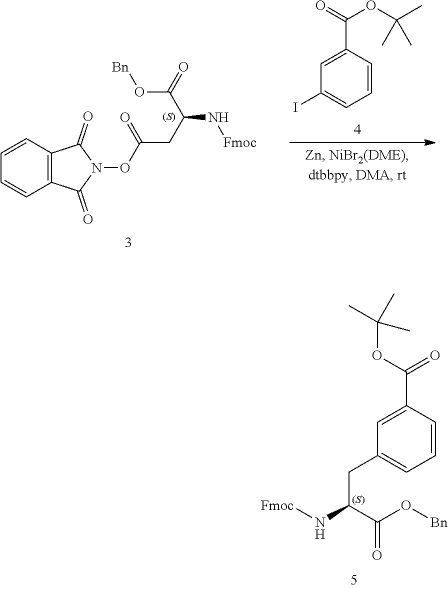

A solution of dibromonickel; 1,2-dimethoxyethane (2.85 g, 9.25 mmol, 7.03 e⁻² eq) and 4-tert-butyl-2-(4-tert-butyl-2-pyridyl)pyridine (2.48 g, 9.24 mmol, 7.03 e⁻² eq) in DMA (500 mL) was stirred at room temperature for 0.5 hr. Compound 3 (68.0 g, 115 mmol, 8.75 e⁻¹ eq), compound 4 (40.0 g, 131 mmol, 1.00 eq), dodecane (15.0 g, 88.0 mmol, 20.0 mL, 0.670 eq), Zn (30.0 g, 458 mmol, 3.49 eq) were added to the reaction. The mixture was stirred at 25° C. for 2.5 hrs. LC-MS (EW23957-16-P1A) showed Compound 3 was consumed completely, and desired mass was detected (R_{f}=1.126 min). The reaction mixture was quenched by the addition of HCl (500 mL). The resulting mixture was extracted with EtOAc (500 mL*3). The combined organic layers were washed with saturated brine (500 mL*3), dried over Na₂SO₄, filtered and concentrated under reduced pressure to give a residue. The crude product was purified by reversed-phase HPLC (0.1% FA condition). Compound 5 (35.0 g, 60.5 mmol, 46.0% yield) was obtained as a yellow oil. LCMS Rₜ=1.126 min, m/z=623.2 (M+46)⁺. ¹HNMR (CDCl₃, 400 MHz) δ: 7.88 (d, J=7.6 Hz, 1H), 7.80-7.76 (m, 3H), 7.56 (t, J=7.2 Hz, 2H), 7.42-7.29 (m, 10H), 7.20-7.19 (m, 1H), 5.37 (d, J=8.0 Hz, 1H), 5.17 (d, J=2.8 Hz, 2H), 4.81-4.71 (m, 1H), 4.42-4.36 (m, 2H), 4.13-4.20 (m, 1H), 3.21-3.17 (m, 2H), 1.58 (s, 9H).

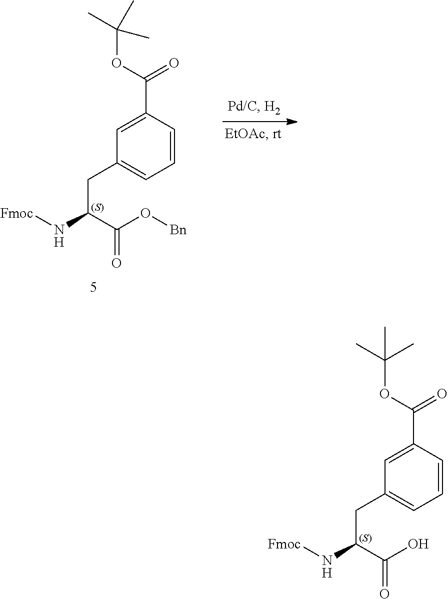

To a solution of compound 5 (35.0 g, 60.5 mmol, 1.00 eq) in EtOAc (50.0 mL) was added Pd/C (3.50 g, 10.0% purity). The mixture was stirred at 25° C. for 2 hrs under H₂ (15 psi) atmosphere. LC-MS (EW23957-19-P1A) showed that compound 5 was consumed, and desired mass was detected (Rₜ=0.991 min). The mixture was filtered, and the filtrate was concentrated in vacuo. The mixture was purified by reversed-phase HPLC (FA condition). The final product (23.5 g, 48.0 mmol, 79.2% yield, 99.6% purity) was obtained as a white solid. LCMS Rₜ=1.088 min, m/z=510. (M+23)⁺. HPLC Rₜ=3.51 min, purity: 99.6%. ¹HNMR (DMSO, 400 MHz) δ: 7.87-7.84 (m, 3H), 7.80-7.74 (m, 2H), 7.62-7.51 (m, 3H), 7.41-7.37 (m, 3H), 7.31-7.23 (m, 2H), 4.24-4.20 (m, 1H), 4.20-4.19 (m, 2H), 4.19-4.14 (m, 1H), 3.18-3.13 (m, 1H), 2.98-2.91 (m, 1H), 1.51 (s, 9H). SFC: Chiral purity: 99.5%.

### Example 1-c. Synthesis of TfeGA

### Step 1: (S)-2-(((benzyloxy)carbonyl)amino)-3-((tert-butoxycarbonyl)amino)propanoic acid

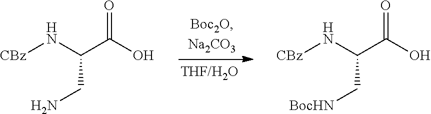

A mixture of (S)-3-amino-2-(((benzyloxy)carbonyl)amino)propanoic acid (20 g, 84 mmol), (Boc)₂O (36.6 g, 168 mmol) and Na₂CO₃ (17.8 g, 168 mmol) in THF (400 mL) and water (250 mL) was stirred at room temperature for 3 h. The mixture was titrated with 1N HCl until the pH reached 3˜4. The aqueous phase was extracted with DCM (3×500 mL). The organic layers were collected, dried, and concentrated to afford the crude product (28.5 g, 100% yield) as a white solid. MS (ESI): m/z=361.1 [M+Na]⁺.

### Step 2: Benzyl (S)-2-(((benzyloxy)carbonyl)amino)-3-((tert-butoxycarbonyl)amino) propanoate

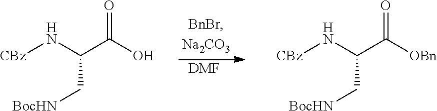

A mixture of (S)-2-(((benzyloxy)carbonyl)amino)-3-((tert-butoxycarbonyl)amino) propanoic acid (28.5 g, 84.3 mmol), benzyl bromide (21.6 g, 126.5 mmol) and Na₂CO₃ (17.8 g, 168.7 mmol) in DMF (500 mL) was stirred at room temperature for 3 h. The reaction mixture was diluted with ethyl acetate (2 L), washed with brine (5×500 mL), dried over Na₂SO₄ and filtered. The filtrate was concentrated and the crude mixture was purified by silica gel column chromatography (eluted with hexane/ethyl acetate=4:1, V/V) to afford the product (35.5 g, 99% yield) as a colorless oil. MS (ESI): m/z=451.1 [M+Na]⁺.

### Step 3: Benzyl (S)-3-amino-2-(((benzyloxy)carbonyl)amino)propanoate

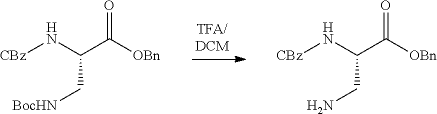

A mixture of benzyl (S)-2-(((benzyloxy)carbonyl)amino)-3-((tert-butoxycarbonyl) amino)propanoate (35.5 g, 82.9 mmol) in TFA (100 mL) and DCM (100 mL) was stirred at room temperature for 3 h, then solvent was removed under reduced pressure. The mixture was titrated with sat. NaHCO₃until the pH reached 8-9. The aqueous phase was extracted with DCM (3×1000 mL). The organic layers were combined, dried, and concentrated to afford the product (26.8 g, 98.5% yield) as a colorless oil. MS (ESI): m/z=329.1 [M+H]⁺.

### Step 4: Benzyl (S)-2-(((benzyloxy)carbonyl)amino)-3-((2-(tert-butoxy)-2-oxoethyl)amino) propanoate

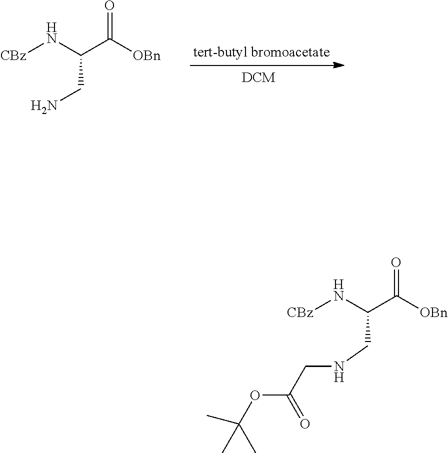

A mixture of benzyl (S)-3-amino-2-(((benzyloxy)carbonyl)amino)propanoate (13.5 g, 41.1 mmol) and tert-butyl 2-bromoacetate (8.03 g, 41.1 mmol) in DCM (250 mL) was stirred at room temperature for 2 days. Et₂NH (3 g, 41.1 mmol) was added and the reaction mixture was stirred at room temperature for 1 h. The mixture was titrated sat. NaHCO₃ until pH reached 8-9. The aqueous phase was extracted with DCM (3×500 mL). The organic layers were combined, dried, and concentrated. The crude mixture was purified by silica gel column chromatography (eluted with hexane/ethyl acetate=4:1, V/V) to afford the product (8.2 g, 45% yield) as a colorless oil. MS (ESI): m/z=443.2 [M+H]⁺.

### Step 5: Benzyl (S)-2-(((benzyloxy)carbonyl)amino)-3-((2-(tert-butoxy)-2-oxoethyl) (2,2,2-trifluoroethyl)amino)propanoate

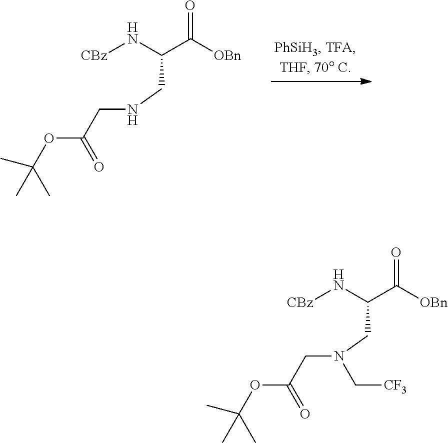

To an oven-dried 500 ml round-bottomed flask fitted with a water condenser under an argon atmosphere (balloon) was added tetrahydrofuran (400 mL) and benzyl (S)-2-(((benzyloxy)carbonyl)amino)-3-((2-(tert-butoxy)-2-oxoethyl)amino)propanoate (11.5 g, 26 mmol) as the free base. The reaction flask was heated in an oil bath at 70° C. Phenylsilane (14.0 g, 130 mmol) in THF (25 mL) was added immediately via syringe, followed by TFA (14.1 g, 123.6 mmol) in THF (25 mL). The reaction was stirred at reflux for 4 h. The mixture was concentrated and titrated with sat. NaHCO₃ until pH reached 8-9. The aqueous phase was extracted with DCM (3×500 mL). The organic layers were combined, dried, and concentrated. The crude mixture was purified by silica gel column chromatography (eluted with hexane/ethyl acetate=6:1, V/V) to afford the product (11.2 g, 82% yield) as a colorless oil. MS (ESI): m/z=525.0 [M+H]⁺.

### Step 6: (S)-2-((((9H-fluoren-9-yl)methoxy)carbonyl)amino)-3-((2-(tert-butoxy)-2-oxoethyl)(2,2,2-trifluoroethyl)amino)propanoic acid

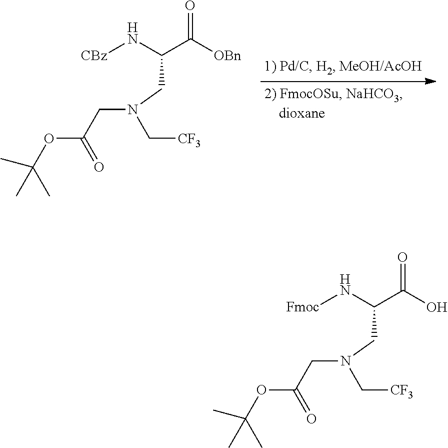

A mixture of benzyl (S)-2-(((benzyloxy)carbonyl)amino)-3-((2-(tert-butoxy)-2-oxoethyl)(2,2,2-trifluoroethyl)amino)propanoate (5.5 g, 10.5 mmol) and palladium on carbon (3 g, 10%) in MeOH (200 mL) and AcOH (8 mL) was attached to a hydrogenation apparatus. The system was evacuated and then refilled with hydrogen. The mixture was stirred at room temperature for 16 h. The reaction mixture was filtered. The filtrate was concentrated and re-dissolved in dioxane (150 mL) and water (150 mL). FmocOSu (3.36 g, 10 mmol) and NaHCO₃ (4.41 g, 52.5 mmol) were added. The mixture was stirred at room temperature for 16 h. The mixture was titrated with 0.5 N HCl until pH reached 4. The aqueous phase was extracted with ethyl acetate (3×500 mL). The organic layers were combined, dried, and concentrated. The crude mixture was purified by combiflash on C18 (0-80% MeCN/H₂O) to give the product (3.5 g, 64% yield) as a white solid. MS (ESI): m/z=523.0 [M+H]⁺. ¹H NMR, 400 MHz, DMSO-d6, δ 12.73 (s, 1H); 7.90 (d, J=7.6 Hz, 2H); 7.72 (d, J=7.6 Hz, 2H); 7.59 (d, J=8 Hz, 1H); 7.42 (t, J=7.2 Hz, 2H); 7.30 (t, J=7.2 Hz, 2H); 4.29-4.21 (m, 3H); 4.14-4.09 (m, 1H); 3.54-3.42 (m, 4H); 3.19 (dd, J1=14.2 Hz, J2=4.8 Hz, 1H); 3.00-2.95 (m, 1H); 1.42 (s, 9H).

### Example 2. Provided Technologies can Provide Improved Properties and/or Activities

Among other things, the present disclosure provides technologies for modulating properties and/or activities of products such as peptides through, e.g., incorporation of certain amino acid residues. As demonstrated herein, various amino acid residues, such as TfeGA and 3COOHF, can provide certain modulated properties (e.g., improved lipophilicity (in some instances, assessed by Log D values)) and/or activities compared to comparable residues (e.g., Aad, Asp, etc.) without significant negative impact on other properties and/or activities (e.g., solubility, binding to target proteins like beta-catenin, etc.). For example, as shown in Table E1, in some embodiments, the present disclosure provides peptides with increased lipophilicity. In some embodiments, the present disclosure provides peptides with improved Log D. In some embodiments, improvements are achieved by replacing an amino acid residue whose side chain comprises an acidic group (e.g., —COOH) but no amino group (e.g., Asp, Aad, etc.; a peptide comprising such an amino acid residue may be utilized in some embodiments as a reference peptide (e.g., when compared to a peptide in which such an amino acid residue (e.g., Asp, Aad, etc.) is replaced (e.g., with TfeGA) and which is otherwise identical)) with an amino acid residue whose side chain comprises an acidic group (e.g., —COOH) and an amino group (e.g., —N(R′)—)(e.g., TfeGA). In some embodiments, provided technologies can improve lipophilicity, Log D, etc. without undesirably impacting other properties and/or activities (e.g., solubility, target binding, etc.). In some embodiments, one or more additional properties and/or activities in addition to Log D were improved compared to reference peptides (e.g., peptides comprising an amino acid residue whose side chain comprises an acidic group (e.g., —COOH) but no amino group (e.g., Asp, Aad, etc.) but are otherwise identical).

In some embodiments, Log D was measured using a CHI Log D procedure: 3 uL of a 0.2 mM solution of peptide in 90% DMSO was injected onto a Phenonenex Gemini 3 um C18 110A column (50×3 mm), eluting with a gradient of 50 mM ammonium acetate pH 7.4 and acetonitrile. The retention time was compared to a standard calibration solution of 10 compounds to derive CHI Log D:

| Compound | Gradient tR at pH 7.4 | CHI at pH 7.4 |
|---|---|---|
| Theophylline | 1.671 | 18.40 |
| Phenyltetrazole | 1.768 | 23.60 |
| Benzimidazole | 1.911 | 34.30 |
| Colchicine | 2.132 | 42.00 |
| Phenyltheophylline | 2.271 | 51.20 |
| Acetophenone | 2.475 | 65.10 |
| Indole | 2.642 | 71.50 |
| Propiophenone | 2.734 | 77.40 |
| Butyrophenone | 2.932 | 87.50 |
| Valerophenone | 3.113 | 96.20 |

In some embodiments, solubility was assessed as follows: 50 uM peptide was incubated in 99.5% PBS/0.5% DMSO at 37° C. for 15 min. After ultracentrifugation of the PBS solution, the supernatant was analyzed by HPLC and compared to an HPLC injection 50 μM peptide DMSO solution. Solubility was determined by: [(Area of PBS peak)/(Area of DMSO peak)]*50 uM.

TABLE E1
Certain data for several peptides.

| | | bCat FP | | | | |
|---|---|---|---|---|---|---|
| Compound | | IC50 | | Solubility | NanoBRET | |
| ID | Notes | (nM) | LogD | (uM) | IC50 | |
| I-612 | only staple | 64 | 2.4 | >25 | | |
| | isomer isolated | | | | | |
| I-608 | Staple isomer 1 | 211 | 2.5 | >25 | n.d. | |
| I-609 | Staple isomer 2 | 80 | 2.5 | >25 | n.d. | |
| I-610 | Staple isomer 1 | 54 | 2.6 | >25 | n.d. | |
| I-611 | Staple isomer 2 | 55 | 2.6 | >25 | n.d. | |
| I-686 | Staple isomer 1 | <5 | 2.0 | >25 | 7.5 | uM |
| I-687 | Staple isomer 2 | 12 | 2.0 | >25 | n.d. | |
| I-761 | Staple isomer 1 | <5 | 2.2 | >25 | 5 | uM |

| | | | | | |
|---|---|---|---|---|---|
| I-762 | Staple isomer 2 | 6 | 2.2 | >25 | n.d. |

n.d.: not determined

### Example 3. Various Provided Peptides can Bind to Beta-Catenin

As those skilled in the art will appreciate, many technologies can be utilized in accordance with the present disclosure to assess binding to targets such as beta-catenin. Certain useful technologies and results are described below as examples.

In some embodiments, an assay is fluorescence polarization. A useful protocol is described below as an example.

Fluorescence polarization IC50: Using the Mosquito (SPT) peptide solutions were 3-fold serially diluted in 90% DMSO and 40 nL of titrated peptide was added into 20 uL buffer (50 mM HEPES, pH 7.5, 125 mM NaCl, 2% glycerol, 0.5 mM EDTA, 0.05% v/v pluronic acid) for final concentrations of 10 uM to 5 nM plated by Multidrop™ Combi (Thermo Scientific) into a black polystyrene 384-well plate (Corning). Probe solution (10 nM full-length beta-Catenin (Uniprot ID P35222), mixed with 10 nM 5FAM labeled TCF4 residues 10-53 (Uniprot ID Q9NQB0) peptide in buffer) was prepared and 20 uL per well was plated using a Multidrop™ Combi (Thermo Scientific). The plate was incubated protected from light for 60 minutes at 20° C. prior to read. Reads were performed on a CLARIOstar plate reader (BMG Labtech) in duplicate, and data were fitted to a 1:1 binding model with hill slope using an in-house script. All provided concentrations are final concentrations.

In some embodiments, an assay is an Alphascreen binding assay. A useful protocol is described below as an example.

Alphascreen binding assay: Using the AlphaScreen Histidine (Nickel Chelate) Detection Kit (PerkinElmer), peptide solutions were prepared in buffer (50 mM Tris pH 8.0, 250 mM NaCl, 2% glycerol, 0.03% Tween-20, 0.01% TritonX-100, 0.1% BSA w/v) using a 3-fold serial dilution from 10 uM to 5 nM. Probe solution (65 nM full-length B-Catenin (Uniprot ID P35222), mixed with 10 nM biotin labeled TCF4 residues 10-53 (Uniprot ID Q9NQB0) peptide in buffer) was prepared and 4 uL per well was plated in a white polystyrene 384-well plate (Corning). Equal volume of the titrated peptide was added to the plate and incubated for 15 minutes at 20° C. Then 4 uL of donor and 4 uL acceptor beads at 10 ug/mL were added to the plate, for a 16 uL reaction pool and protected from light for 60 minutes at 20° C. prior to read. Reads were performed on a CLARIOstar plate reader (BMG Labtech) in duplicate, and data were fitted to a 1:1 binding model with hill slope using an in-house script. All provided concentrations are final concentrations.

### Example 4. Provided Technologies can Modulate Interactions with Beta-Catenin in Cells

Various technologies may be utilized to assess properties and/or activities of provided compounds, e.g., stapled peptides, in cells. In some embodiments, a useful assay is Nano-BRET target engagement assay that assesses beta-catenin/TCF4 engagement. A useful protocol is described below as an example.

On Day 1, HEK293 cells were seeded. Use cells at ˜ 70% confluency. Trypsinize cells without washing with PBS as these cells can be fragile and come off easily. (e.g. 5 ml trypsin/75 flask for 2-5 min @ Rm Temp). Quench trypsin with 10 mL MEM media. Transfer cells to a falcon tube. Spin down @ 250 g for 5 minutes at Rm Temp. Discard SN. Gently re-suspend the cells in 10 mL MEM media. Count the cells twice and calculate how many cells are needed. Plate Parental HEK293 Cell Line at 7 M cells/12 ml/75 cm2 flask using MEM media. Rock plate a couple of times to disperse cells evenly. Incubate at 37° C., 5% CO2 for 5 hours. Cells should be evenly spread and about 70% confluent after 5h.

Transfection of Nano-BRET constructs (B-cat-Halo & TCF4-Luc): Allow Fugen-HD transfection reagent to reach room temperature. Mix by inverting tube, if precipitate is visible, warm up to 37° C. and them cool to Rm Temp. Check Flasks under microscope for confluency of cells (70-80%). Add LiCl to flask containing cells (LiCl 30 mM working concentration—LiCl can be a GSK3 inhibitor and reduce beta-catenin degradation). Prepare the transfection mix in a tube containing Assay media based on the manufacturer instruction (see below table for an example): Transfection mix preparation

| | | # of | | DNA | FuGene | Opti-MEM |
|---|---|---|---|---|---|---|
| # | Constructs | Flasks | Ratio | (ug) | (6 ul/w) | (ul) |
| 1-a | Bcat-Halo | 1 | 4 | 12.8 | 48 | 736 |
| 1-b | TCF4-Luc | 1 | 1 | 3.2 | | |

Add FuGene last and gently mix. Don't vortex. Incubate transfection mix at RT for 10-15 minutes. If more than one target pair is going to be tested, calculate the amounts of transfection mix using the above table for other construct pairs. Gently add 700 uL of transfection mix per flask and gently rock the plate a couple of times. Incubate cells at 37° C., 5% CO2 for 18-24 hours.

On Day 2, transfected cells were harvested and re-plated in 96-well plates. Collect media from flask in a Falcon tubes. This was to harvest the floaters as they may still be viable and transfected. Trypsinize cells without washing with PBS (5 ml trypsin/Flask). Quench trypsin with 5 mL of MEM media. Collect cells and add to falcon tube. Wash the flask with 5-10 ml of MEM media and add to falcon tube. Spin down @ 250 g for 5 minutes at Rm Temp. Discard SN. Gently Re-suspend cells in 5 ml Assay media (optionally containing LiCl). Count the cells twice and calculate the average count. Dispense 80 uL of cell suspension per each well of 96-well plate (20,000 cells/80 uL/well) (use plate such as Corning Solid White Flat Bottom TC-treated plate). LiCl at 30 mM concentration. Typically no cells to wells at the edge of the plate, instead add Assay media to these wells. Incubate cells at 37° C., 5% CO2 until peptide dilutions are ready.

Preparation of addition of compounds: Prepare a 10× Dose curve plate by serial dilution. Prepare Row A of Dose Curve Plate in Assay Media only (for 20 uM top conc. We need a 1/50 dilution of 10 mM). Prepare Assay Media plus DMSO (mix 9.8 part media+0.2 part DMSO) (e.g. 9.8 ml of Assay Media+0.2 ml of DMSO). 1:2 serial dilutions: Transfer Vol needed (50 uL) from Row A to Row B, mix 3 times, discard the tips. Continue 1:2 dilutions in each row by transferring 50 uL from higher dilution to lower dilution (e.g. 50 uL from Row B to C, etc.) Discard Vol needed (50 uL) from the last dilution (Row F).

### A Useful Dose Curve Plate Map (6 Point; Typically Use a V-Bottom or U-Bottom Non-Treated 96-Well Plate)

| Pep 1 | Pep 2 | Pep 3 | Pep 4 | Pep 5 | Pep 6 | Pep 7 | Pep 8 | Pep 9 | Pep 10 | Pep 11 | Pep 12 |
|---|---|---|---|---|---|---|---|---|---|---|---|
| 1 | 2 | 3 | 4 | 5 | 6 | 7 | 8 | 9 | 10 | 11 | 12 |
| 200 | | | | | | | | | | | |
| 100 | | | | | | | | | | | |
| 50 | | | | | | | | | | | |
| 25 | | | | | | | | | | | |
| 12.5 | | | | | | | | | | | |
| 6.25 | | | | | | | | | | | |
| Empty | | | | | | | | | | | |
| Empty | | | | | | | | | | | |

### A Useful Dose Curve Plate Map (3 Point; Typically Use a V-Bottom or U-Bottom Non-Treated 96-Well Plate)

| Pep 1 | Pep 2 | Pep 3 | Pep 4 | Pep 5 | Pep 6 | Pep 7 | Pep 8 | Pep 9 | Pep 10 | Pep 11 | Pep 12 |
|---|---|---|---|---|---|---|---|---|---|---|---|
| 1 | 2 | 3 | 4 | 5 | 6 | 7 | 8 | 9 | 10 | 11 | 12 |
| 200 | | | | | | | | | | | |
| 100 | | | | | | | | | | | |
| 50 | | | | | | | | | | | |
| Empty | | | | | | | | | | | |
| 200 | | | | | | | | | | | |
| 100 | | | | | | | | | | | |
| 50 | | | | | | | | | | | |
| Empty | | | | | | | | | | | |
| Pep 13 | Pep 14 | Pep 15 | Pep 16 | Pep 17 | Pep 18 | Pep 19 | Pep 20 | Pep 21 | Pep 22 | Pep 23 | Pep 24 |

Prepare Assay Plates+Peptide+Ligand

Certain wells are without cells, containing 100 uL of Assay media to avoid edge effect. Transfer 10 uL of each peptide dilution to corresponding well in Assay plate [containing cells (80 uL)] to achieve 1:10 final working dilution of peptides. Dilute HaloTag R NanoBRET™ 618 Ligand 1:100 in Assay media (Opti-MEM+4% FBS). Add 10 uL of Ligand per well of Assay plate (80 uL cell+10 uL compound+10 uL Ligand). Adjust the volume in control wells by adding Media or Media+DMSO to corresponding wells. Plus ligand=Add 10 uL Media+DMSO, or Media (for Media only control). No ligand=Add 10 uL Media+DMSO, or Media and add another 10 uL of media to top up to 100 uL. Incubate at 37° C., 500 CO2 overnight. A useful assay plate map—(use Corning Solid White Flat Bottom TC-treated plate) as example (0.5% DMSO in assay plate):

| Dose | | | | | | | | | | | | | |
|---|---|---|---|---|---|---|---|---|---|---|---|---|---|
| (uM) | A | 1 | 2 | 3 | 4 | 5 | 6 | 7 | 8 | 9 | 10 | 11 | 12 |
| 20 | B | Peptide 1 | | | Peptide 2 | | | Peptide 3 | | | Peptide 4 | | |
| 10 | C | | | | | | | | | | | | |
| 5 | D | | | | | | | | | | | | |
| 2.5 | E | | | | | | | | | | | | |
| 1.25 | F | | | | | | | | | | | | |
| 0.625 | G | | | | | | | | | | | | |

| | | |
|---|---|---|
| H | Media + DMSO (No Ligand) | Media + DMSO + Ligand |

In some embodiments, peptide 1 is 1-797, and peptide 2 is 1−686.

On Day 3, fluorescence was read with Nano-BRET substrates. Remove plates from incubator to allow to reach to RT (30 min). Also equilibrate CTG reagent to Rm Temp. Dilute Nano-BRET substrate 1:100 in Assay media. Add 25 uL of diluted substrate to each well and shake for 30 seconds. Read on ClarioSTAR or GloMAX right away (within 10 min). Donor emission @ 460 nm. Acceptor emission @ 618 nm. Use the same plate to measure cell viability (Cell Titer-Glo-2.0 (CTG) Viability test). After reading BRET signal, add CTG reagent to each well at 1:2 ratio and shake on orbital shaker for 2 min. Incubate at Rm Temp for 10-30 min. Read luminescence on ClarioSTAR or GloMAX. Analysis was performed using Prism. Non-linear regression. Log (inhibitor) vs response—variable slope (four parameters). For CTG data linear values (not logarithmic).

Certain results were presented below:

### CTG (CellTiter Glo):

| Conc. | Relative to Media | | | | | |
|---|---|---|---|---|---|---|
| (uM) | I-822 | | | I-797 | | |
| 20 | 82 | 83 | 92 | 92 | 91 | 92 |
| 10 | 90 | 90 | 92 | 100 | 100 | 102 |
| 5 | 94 | 95 | 95 | 102 | 100 | 104 |
| 2.5 | 99 | 98 | 98 | 103 | 107 | 105 |
| 1.25 | 94 | 98 | 99 | 104 | 103 | 101 |
| 0.625 | 100 | 102 | 104 | 104 | 104 | 103 |
| Media | 109 | 96 | 96 | | | |
| Avg of media | 100 | | | | | |

### BRET (Donor: 450 BP; Acceptor: 600 LP; Integration 0.3 s; Reading 1)

### Donor 450 BP

| | 1 | 2 | 3 | 4 | 5 | 6 | 7 | 8 | 9 | 10 | 11 | 12 |
|---|---|---|---|---|---|---|---|---|---|---|---|---|
| A | | I-822 | | | I-797 | | | | | | | |
| B | | 59340 | 52650 | 45840 | 58480 | 55870 | 47990 | | | | | |
| C | | 54740 | 47760 | 46640 | 58470 | 25870 | 40350 | | | | | |
| D | | 56000 | 46130 | 45780 | 38850 | 29900 | 47500 | | | | | |
| E | | 54130 | 32300 | 33550 | 34130 | 22720 | 40100 | | | | | |
| F | | 38290 | 28720 | 18860 | 21810 | 34520 | 71060 | | | | | |
| G | | 78090 | 58990 | 23030 | 25180 | 35670 | 47630 | | | | | |
| H | | | | | | | | | | | | |

### Acceptor 600 LP

| | 1 | 2 | 3 | 4 | 5 | 6 | 7 | 8 | 9 | 10 | 11 | 12 |
|---|---|---|---|---|---|---|---|---|---|---|---|---|
| A | | I-822 | | | I-797 | | | | | | | |
| B | | 450 | 450 | 280 | 860 | 660 | 640 | | | | | |
| C | | 490 | 470 | 470 | 790 | 350 | 470 | | | | | |
| D | | 630 | 600 | 430 | 510 | 420 | 630 | | | | | |
| E | | 740 | 360 | 420 | 460 | 220 | 510 | | | | | |
| F | | 470 | 380 | 260 | 300 | 410 | 860 | | | | | |
| G | | 1120 | 720 | 260 | 410 | 440 | 530 | | | | | |
| H | | | | | | | | | | | | |

### % Inhibition:

| Concentration (uM) | I-822 | | | I-797 | | |
|---|---|---|---|---|---|---|
| 20 | 52.9 | 40.7 | 71.4 | −36.7 | −0.3 | −19.5 |
| 10 | 35.7 | 24.5 | 21.5 | −21.7 | −21.9 | 1.7 |
| 5 | 6.8 | −15.3 | 30.1 | −16.9 | −28.4 | −18.6 |
| 2.5 | −23.7 | 8.1 | −9.2 | −21.3 | 26.5 | −11.7 |
| 1.25 | −6.1 | −18.2 | −25.1 | −24.8 | −1.1 | −4.0 |
| 0.625 | −32.1 | −5.3 | 6.3 | −56.5 | −6.9 | 8.3 |

In some embodiments, it was observed that amino acids with non-polar side chains (e.g., Leu) at X¹⁰ may provide improved affinity compared to amino acids with polar side chains (e.g., Asn). For example, see I-761 (X¹⁰=Asn, NanoBRET IC50=5 uM) and I-849 (X¹⁰=Leu, NanoBRET IC50=2.3 uM).

Among other things, NanoBRET data (and/or data from certain other assessments) indicated on-target activity. In some embodiments, optimization of certain residues, e.g., one or more residues at X², X⁵, X⁹, X² and/or X¹³, can improve binding affinity (e.g., I-926 to I-921).

Certain data of various peptides are presented in Table E2 as examples.

Various technologies may be utilized to deliver provided compounds, including various peptides, and compositions. In some embodiments, lipids are utilized to deliver provided compounds and compositions. In some embodiments, lipids form positively charged complexes with provided compounds, e.g., various peptides. In some embodiments, lipids non-covalently associate with provided compounds. In some embodiments, lipids non-covalently associate with provided compounds to form positively charged complexes. In some embodiments, the present disclosure provides a composition comprising a provided compound, e.g., a stapled peptide, and a lipid. In some embodiments, a lipid is SAINT-Protein (ST-Protein, Synvolux). Certain results are present below. As demonstrated, lipids can improve activities of various peptides:

### Nano-BRET:

| Abs IC50 (uM) | I-1001 (neg.) | I-849 | I-922 | I-762 | I-993 |
|---|---|---|---|---|---|
| Peptide only | inactive | 5 | 6 | 11 | 7 |
| Peptide + ST-Protein | inactive | 0.7 | 1 | 0.7 | 0.9 |

### TCF Reporter Assay:

| Abs IC50 (uM) | I-1001 (neg.) | I-849 | I-922 | I-762 | I-993 |
|---|---|---|---|---|---|
| Peptide only | inactive | 5 | 5 | 7 | 5 |
| Peptide + ST-Protein | inactive | 0.2 | 0.2 | 0.3 | 0.2 |

Assay were performed essentially as described above. Prepare 100× peptide dose curve in DMSO, then add fixed amount to HEPES in Pool B. Add SAINT-Protein (StPhD) in HEPES to achieve 10× mix, incubate at Rm Temp and add 10 uL to 90 uL of Cells+Ligand in cell culture plate.

| | | | | Conditions | | | | |
|---|---|---|---|---|---|---|---|---|
| | | | | Pool A | Pool B | | | |
| | Dose (uM) | | Final Vol | StPhD | Peptide prep | HEPES | Assay | % StPhD in |
| Ratio of | in final | Dose prep | (Pep + | ratio | in DMSO | minus | media + | 400 uL |
| Pep:StPhD | plate | (10X) | StPhD) | (uL) | (uL) | peptide (uL) | LiCl | media |
| peptide | 20 | 200 | 50 | 0 | 5.0 | 45.0 | 450 | 0.0 |
| only | 10 | 100 | 50 | 0 | 5.0 | 45.0 | 450 | 0.0 |
| | 5 | 50 | 50 | 0 | 5.0 | 45.0 | 450 | 0.0 |
| | 3 | 25 | 50 | 0 | 5.0 | 45.0 | 450 | 0.0 |
| | 1 | 13 | 50 | 0 | 5.0 | 45.0 | 450 | 0.0 |
| | 0.6 | 6 | 50 | 0 | 5.0 | 45.0 | 450 | 0.0 |
| 2.5:1 | 20 | 200 | 50 | 14.3 | 5.0 | 30.7 | 450 | 2.9 |
| (Pool B:Pool A) | 10 | 100 | 50 | 14.3 | 5.0 | 30.7 | 450 | 2.9 |
| | 5 | 50 | 50 | 14.3 | 5.0 | 30.7 | 450 | 2.9 |
| | 3 | 25 | 50 | 14.3 | 5.0 | 30.7 | 450 | 2.9 |
| | 1 | 13 | 50 | 14.3 | 5.0 | 30.7 | 450 | 2.9 |
| | 0.6 | 6 | 50 | 14.3 | 5.0 | 30.7 | 450 | 2.9 |

Transfer 10 uL/96 well corresponding wells which contain seeded cells in 80 uL media. To prepare DMSO control add 5 uL DMSO to 50 uL HEPES (same as peptide only prep). Add 10 uL of 1/100 dilution of ligand at the end (total volume is 20,000 cells/100 uL). Incubate at 37° C., overnight. Process data next day based on protocol sheet.

The “Rokhand-NanoBRET-Bcat-Protocal-Saint protein” Excel file contain CTG and GloMax reading data. What peptides were they for? It also mentioned the following peptides—were they tested? If yes, where are the data?

| # | ID | Volume (ul) |
|---|---|---|
| 1 | I-922 | 40 |
| 2 | I-762 | 40 |
| 3 | I-993 | 40 |
| 4 | I-1001 | 40 |
| 7 | I-849 | 40 |

### Example 5. Provided Technologies can Modulate Beta-Catenin Functions in Cells

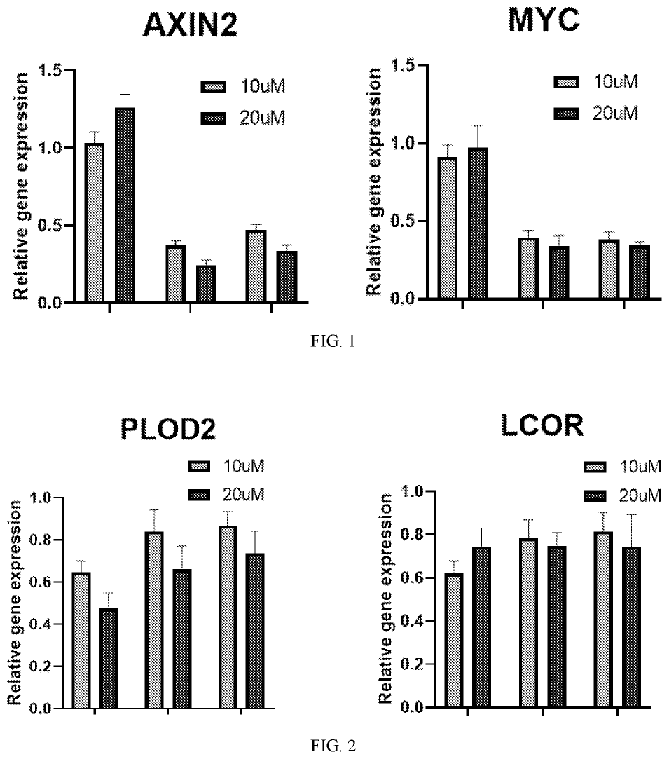

As demonstrated in FIG. 1, provided technologies can modulate functions of beta-catenin in cells. Among other things, provided technologies can modulate expression levels of various genes regulated by beta-catenin/TCF. In some embodiments, provided technologies can reduce expression of certain genes such as AXIN2 and MYC. 1-796 is a negative control. A useful cell-based assay is described below.

A protocol for a gene expression assay:

Cell Line: The SW620 cell line was purchased from ATCC (ATCC CCL-227) and was grown in RPMI-1640 medium supplemented with 10% FBS (ThermoFisher Catalog #11875093, Catalog #10082147). Cell line was maintained in an incubator with a 5% CO2 atmosphere at 37° C.

Materials:
RNeasy Mini Kit Qiagen Catalog number: 74104
Primer/Probes from Applied Biosystems—Life Technology (20X)
Applied Biosystems High-Capacity cDNA Reverse Transcription Kit, Catalog number: 4368814
Applied Biosystems TaqMan Fast Advanced Master Mix, Catalog number: 4444557

Peptide Treatment:
1. Remove medium from cell flask by aspiration, trypsinize (0.05% Trypsin) and allow cells to dissociate from the flask bottom.
2. Neutralize trypsin using cell culture medium, count cells to estimate density and re-suspend to a final density of 7.5×104 cells/mL in 4% FBS containing cell culture medium.
3. Plate 2 mL of cells (150,000 cells) into a well of a six-well plate.
4. Allow the cells to attach and recover for overnight at 37° C., 5% CO₂.
5. 24 hours after seeding cells treat with corresponding amounts of peptide normalizing the DMSO background concentrations to 0.2%.
6. 48 hours after treatment remove medium from treated cells by aspiration, trypsinize (0.05% Trypsin) and allow cells to dissociate from the plate bottom.
7. Neutralize trypsin using cell culture medium, count cells to estimate density and re-suspend in cold PBS.
8. Spin cells down and remove PBS to move forward with RNA extraction.

RNA Extraction:
RNA Extraction was carried out using the Qiagen RNeasy Mini Kit and the protocol provided.
1. Disrupt cells Disrupt the cells by adding Buffer RLT.
2. Add 1 volume of 70% ethanol to the homogenized lysate, and mix well by pipetting.
3. Transfer up to 700 uL of the sample, including any precipitate that may have formed, to an RNeasy spin column placed in a 2 mL collection tube (supplied). Close the lid gently, and centrifuge for 15 s at ≥8000×g (≥10,000 rpm).
4. Add 700 uL Buffer RW1 to the RNeasy spin column. Close the lid gently, and centrifuge for 15 s at ≥8000×g (≥10,000 rpm).
5. Add 500 uL Buffer RPE to the RNeasy spin column. Close the lid gently, and centrifuge for 15 s at ≥8000×g (≥10,000 rpm) to wash the spin column membrane.
6. Add 500 uL Buffer RPE to the RNeasy spin column. Close the lid gently, and centrifuge for 2 min at ≥8000×g (≥10,000 rpm).
7. Place the RNeasy spin column in a new 1.5 mL collection tube (supplied). Add 30-50 uL RNAse-free water directly to the spin column membrane. Close the lid gently, and centrifuge for 1 min at ≥8000×g (≥10,000 rpm) to elute the RNA.

cDNA Synthesis:
cDNA synthesis was carried out using the High-Capacity cDNA Reverse Transcription Kit and the protocol provided.
1. Pipette 10 uL of 2×RT master mix into each well of a 96-well reaction plate or individual tube.
2 Pipette 10 uL of RNA sample into each well, pipette up and down two times to mix.
3. Seal the plates or tubes.
4. Briefly centrifuge the plate or tubes to spin down the contents and to eliminate any air bubbles.
5. Place the plate or tubes on ice until you are ready to load the thermal cycler.

### Thermo Cycling Conditions:

| | | |
|---|---|---|
| 1. | Temp: 25° C. | Time: 10 min |
| 2. | Temp: 37° C. | Time: 120 min |
| 3. | Temp: 85° C. | Time: 5 min |

Quantitative Real Time PCR:
1. Samples were prepared using Applied Biosystems TaqMan Fast Advanced Master Mix and the protocol provided.
2. Combine 2× Master Mix and 20× TaqMan Assay with water to bring volume to 18 uL. Transfer the appropriate volume of PCR reaction mix to each well of an optical reaction plate. Each well of reaction will received 1 uL of cDNA.
3. Seal the reaction plate with optical adhesive film then centrifuge briefly to bring the PCR reaction mix to the bottom of the well and eliminate air bubbles.
4. Apply a compression pad to the plate, if required by your real-time PCR system.

### Real-time PCR Cycling Conditions:

| | | |
|---|---|---|
| 1. | Temp 50° C. | Time: 2 min |
| 2. | Temp 95° C. | Time: 2 min |
| 3. | Temp 95° C. | Time: 1 second |
| 4. | Temp 60° C. | Time: 20 seconds |

### Gene Expression Analysis:

Relative qPCR was used calculate the relative expression level of the target gene as compared to ACTIN B their expression is relativized ROX according to the following formula: Relative gene expression=2^{-ΔΔCt} where ΔΔCt=ΔCt (sample)−ΔCt (DMSO control); and ΔCt=target gene Ct—reference gene (Actin B).

The present disclosure further demonstrates that provided technologies can modulate beta-catenin regulated expression without significantly impact beta-catenin-independent expression, e.g., beta-catenin-independent WNT target gene expression. Certain data were presented in FIG. 2.

Activities of provided technologies were also confirmed in TCF reporter assay as described below. Those skilled in the art will appreciate that other suitable reagents may be utilized and various parameters may be adjusted.

DLD1 reporter cell line was generated by using TCF/LEF luciferase Reporter Lentivirus (BPS Bioscience Catalog #79787). Parental DLD1 cells (ATCC@ CCL-221) were transfected with the lentivirus and followed by 3-day puromycin selection. Single clone was selected for Reporter assay.

On Day 1, cultured cells in flasks that are no more than about 60-70% confluent were washed with PBS and typsinized in 3 mL/T75 until cells were free floating. Spin down cells for 5 minutes at 1100RPM. While cells spin, determine the number of plates (3 compounds per plate) needed. After spinning, aspirate the supernatant gently and add 10 mL media (RPMI+4% FBS), and re-suspend and mix the cells gently. Count the cells twice in the cell counter Countess and take average of both the counts, and determine how many cells are needed. After plating cells at 5000 cells/well, seal with aerseal film. 90 uL cells/well. Incubate at 37° C. overnight, tap gently to mix and don't stack plates.

On Day 2, compounds were added. Stock solution was 10 mM. Make the DMSO containing media by adding 36 uL DMSO to 10 mL of media. Make the dilution wells by transferring 100 uL DMSO+Media into rows B-F of a protein LoBind 96-well plate. Row G is empty. Add any extra DMSO+media to row H. Transfer 4 uL of stock solution to Row A. One compound per column. Add 196 uL media to Row A, no DMSO in the media. Concentration of Row A was 200 uM. Mix well and do a 6 pt, 1:2 serial dilution, mix ×10 per transfer and change tips before each transfer. Removed excess volume from final well (row F) and discard. Transfer 10 uL of the compound containing media to cells with a multichannel according to plate maps. Add 60 uL DMSO containing media to the DMSO wells. The edge wells were typically not used in the assay and could be ignored if needed or replaced with PBS, but there typically were liquids in the wells. Cover plates with breathable plate sealer and incubate overnight.

A dilution chart is presented below as an example:

| | | |
|---|---|---|
| Starting Concentration (uM) | 10000 | |
| Media + compound Transfer onto | 10 | |
| cells (uL) | | |
| Total Volume needed/compound, | 60 | <-- BrightGlo(x3) + |
| no extra (uL) | | CTG(x3) |
| High Concentration Stock (uM) | 200 | |
| 100% DMSO to add to 10 mL | 36 | |
| media (uL) | | |
| Final Volume (in cell plate) | 100 | |
| Volume compound in DMSO (uM) | 4 | |
| Volume Media Added on top (uL) | 196 | |
| Volume DMSO + media in wells (uL) | 100 | <--Assumes |
| | | 1:2 dillution |
| Volume media to Xfer (uL) | 100 | <--Assumes |
| | | 1:2 dillution |
| Final DMSO concentration (%) | 0.18 | |

A 96-well plate may be utilized to prepare dilution series for up to 12 compounds, each with several concentrations (e.g., 200, 100, 50, 25, 12.5, 6.25 and 0 uM; can be utilized as 100× depending on final test concentrations).

Useful plate maps of cell plates with samples added as examples. 0 uM is the DMSO control for the whole plate. Edge well filled with 90 uL cells+10 uL DMSO containing media.

| | | | | | | | | | | | | |
|---|---|---|---|---|---|---|---|---|---|---|---|---|
| Concentration | 1 | 2 | 3 | 4 | 5 | 6 | 7 | 8 | 9 | 10 | 11 | 12 |

| (uM) | A | DMSO | | | | | |
|---|---|---|---|---|---|---|---|
| 20 | B | DMSO | Compound #1 | DMSO | Compound #2 | Compound #3 | DMSO |
| 10 | C | | | | | | |
| 5 | D | | | | | | |
| 2.5 | E | | | | | | |
| 1.25 | F | | | | | | |
| 0.625 | G | | | | | | |

| | |
|---|---|
| H | DMSO |

On Day 3, luciferase levels were assessed using Bright-Glo (Promega). Equilibrate Bright-Glo to room temperature before addition. 7 mL of mixed Bright-Gbo per plate. Unused Bright-Glo could be store at −80° C. Reconstituted reagent can be stored for up to 1 month at −80° C., or after one freeze-thaw. When ready to read, remove plates from incubator and let sit at room temperature for 15 minutes. Typically stagger taking out plates if reading multiple plates before adding the Bright-Glo. Add 90 uL/well Bright-Glo and shake gently for 3 minutes. Read luminescence on GloMax immediately using ‘BrightGlo’ programs.

Cellular activity was assessed using CTG. Equilibrate CTG reagents to room temperature. Equilibrate plates to room temp, e.g. 15 min. Use same time length for all plates. Add CTG reagent, 25 uL if using undiluted, or 90 uL if diluted 1 to 4. Shake for 2 min to induce cell lysis. Allow plate to to incubate at RT for 10 min to stabilize lumine sent signal. Read luminesence using program ‘CellTiter-Glo’. Use Prism to find the Absolute IC50 (AbsIC50=X[50]) of each compound.

Certain results are presented below as examples:
Plate 1—DLD1 TCF c2 BrightGlo
% Inhibition

| DMSO | | | | | | | | | |
|---|---|---|---|---|---|---|---|---|---|
| ave | 45325 | | | | | | | | |
| I-762 | | | DMSO | I-1001 | | | I-988 | | |
| 75.7 | 81.9 | 78.7 | 1.1 | −1.2 | 7.5 | 3.5 | 78.1 | 81.3 | 81.9 |
| 56.8 | 70.2 | 65.6 | −2.2 | −3.6 | −4.8 | −7.4 | 61.5 | 72.0 | 76.2 |
| 28.2 | 45.2 | 44.6 | −3.2 | −6.7 | −6.7 | −0.7 | 32.0 | 57.8 | 62.0 |
| 12.9 | 17.4 | 11.9 | −3.4 | −8.0 | −7.6 | −5.0 | 13.3 | 28.8 | 33.4 |
| 12.9 | 16.9 | 5.5 | 1.9 | −7.0 | −2.8 | 6.1 | 5.7 | 22.3 | 21.2 |
| 14.5 | 18.6 | 9.3 | 5.9 | −1.5 | 9.3 | 4.5 | 11.0 | 10.7 | 15.5 |

Plate 2—DLD1 TCF c2 BrightGlo
% Inhibition

| DMSO | | | | | | | | | |
|---|---|---|---|---|---|---|---|---|---|
| ave | 4.52E+04 | | | | | | | | |
| I-999 | | | DMSO | I-1000 | | | I-927 | | |
| 63.2 | 71.4 | 68.9 | 5.6 | 79.5 | 79.5 | 79.7 | 77.1 | 77.4 | 80.3 |
| 42.0 | 49.2 | 49.7 | −1.6 | 57.0 | 59.8 | 61.4 | 68.0 | 70.2 | 72.9 |
| 18.2 | 32.5 | 23.9 | −0.7 | 25.7 | 29.9 | 32.3 | 53.1 | 55.2 | 60.1 |
| 14.3 | 11.9 | 4.4 | −5.7 | 5.5 | 10.2 | 12.4 | 23.9 | 33.7 | 42.0 |
| 1.7 | 8.0 | 4.0 | 1.6 | −0.6 | −1.2 | 1.5 | 8.7 | 15.1 | 18.6 |
| 7.7 | 8.8 | 6.4 | 0.8 | −3.4 | 2.4 | 4.9 | 3.5 | 8.0 | 14.3 |

Plate 3—DLD1 TCF c2 BrightGlo
0inhibition

| DMSO | | | | | | | | | |
|---|---|---|---|---|---|---|---|---|---|
| ave | 5.03E+04 | | | | | | | | |
| I-1050 | | | DMSO | I-1051 | | | I-1079 | | |
| 61.5 | 59.8 | 58.7 | 8.4 | 20.2 | 21.7 | 24.7 | 55.6 | 58.1 | 54.7 |
| 50.8 | 46.4 | 39.4 | 1.4 | 3.9 | 5.0 | −0.5 | 34.2 | 39.4 | 42.0 |
| 25.4 | 24.7 | 12.9 | −2.2 | −4.1 | −5.0 | −11.4 | 7.4 | 16.0 | 26.5 |
| 6.2 | 1.9 | −2.2 | −3.6 | −12.5 | −11.1 | −13.2 | −25.7 | −0.8 | 5.1 |
| 6.8 | −0.9 | −4.5 | −0.8 | −12.2 | −5.2 | −3.8 | 1.4 | −0.6 | 2.6 |
| 13.7 | 5.5 | 6.1 | −3.2 | −1.7 | −3.4 | 0.0 | 1.4 | 6.9 | 6.8 |

Plate 4—DLD1 TCF c2 BrightGlo
The ‘empty’ wells along the edges of the plates was either 200 uL PBS (DLD1 TCF Neg) or cells treated with DMSO only (DLD1 TCF c2)
% Inhibition

| DMSO | | | | | | | | | |
|---|---|---|---|---|---|---|---|---|---|
| ave | 4.84E+04 | | | | | | | | |
| I-1080 | | | DMSO | I-1081 | | | I-1082 | | |
| 51.4 | 56.9 | 51.8 | 4.7 | 67.2 | 69.3 | 72.3 | 76.5 | 77.1 | 79.9 |
| 42.4 | 42.2 | 38.1 | −1.7 | 45.6 | 49.8 | 55.3 | 63.9 | 71.0 | 75.0 |
| 24.9 | 31.0 | 24.4 | −2.9 | 17.5 | 23.8 | 31.0 | 38.5 | 56.2 | 62.0 |
| 14.6 | 19.8 | 7.9 | −1.0 | 4.5 | 7.9 | 15.2 | 17.3 | 36.1 | 37.9 |
| 16.0 | 13.8 | 12.3 | −5.8 | 0.8 | 2.4 | 5.4 | 14.0 | 21.5 | 24.0 |
| 20.2 | 13.7 | 15.3 | 6.8 | 9.2 | 7.1 | 11.5 | 14.1 | 17.4 | 20.3 |

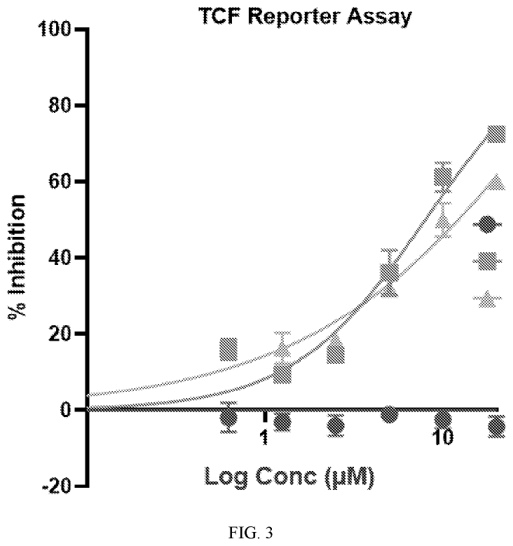

Certain results were presented in FIG. 3.

Table E2. Certain data of various peptide compositions.
Structural information are described in Table E3. Compositions of stapled peptides.
1. ID (may include batch number (e.g., −1)
2: FP EC50 (nM)
3: CHI Log D
4: Solubility (uM)
5: NanoBRET % inh @ 10 uM
6: NanoBRET IC50 (uM)
7: NanoBRET Abs IC50 (uM)
8: DLD1 reporter assay Abs IC50 (uM)
9: alphascreen EC50 (nM)

| 1 | 2 | 3 | 4 | 5 | 6 | 7 | 8 | 9 |
|---|---|---|---|---|---|---|---|---|
| I-1 | | 1.274 | | | | | | 10000 |
| I-2 | | 1.246 | | | | | | 10000 |
| I-3 | | 1.307 | | | | | | 9132 |
| I-4 | | 1.353 | | | | | | 5892 |
| I-5 | | 1.331 | | | | | | 10000 |
| I-6 | | 1.378 | | | | | | 6501 |
| I-7 | | 1.316 | | | | | | 10000 |
| I-8 | | 1.349 | | | | | | 10000 |
| I-9 | | 1.654 | | | | | | 10000 |
| I-10 | | 1.661 | | | | | | 10000 |
| I-11 | | 2.272 | | | | | | 10000 |
| I-12 | | 2.247 | | | | | | 10000 |
| I-13 | | 2.013 | | | | | | 10000 |
| I-14 | | 2.013 | | | | | | 9756 |
| I-15 | | 2.21 | | | | | | 10000 |
| I-16 | | 2.21 | | | | | | 10000 |
| I-17 | | 2.097 | | | | | | 10000 |
| I-18 | | 2.108 | | | | | | 10000 |
| I-19 | | 0.948 | | | | | | 10000 |
| I-20 | | 0.935 | | | | | | 10000 |
| I-21 | | 0.92 | | | | | | 10000 |
| I-22 | | 0.911 | | | | | | 10000 |
| I-23 | | 0.942 | | | | | | 9219 |
| I-24 | | 0.977 | | | | | | 8565.5 |
| I-25 | | 1.174 | | | | | | 7224 |
| I-26 | | 1.248 | | | | | | 8106.5 |
| I-27 | | 1.145 | | | | | | 6824 |
| I-28 | | 1.219 | | | | | | 8459.5 |
| I-29 | | 1.596 | | | | | | 10000 |
| I-30 | | 1.228 | | | | | | 8818.5 |
| I-31 | | 1.245 | | | | | | 7221.667 |
| I-32 | | 1.243 | | | | | | 7351 |
| I-33 | | 1.228 | | | | | | 9978.5 |
| I-34 | | 1.323 | | | | | | 10000 |
| I-35 | | 1.334 | | | | | | 9543.5 |
| I-36 | | 1.13 | | | | | | 8269.5 |
| I-37 | | 1.161 | | | | | | 10000 |
| I-38 | | 1.21 | | | | | | 10000 |
| I-39 | | 1.259 | | | | | | 10000 |
| I-40 | | 1.243 | | | | | | 10000 |
| I-41 | | 1.088 | | | | | | 10000 |
| I-42 | | 1.09 | | | | | | 9337 |
| I-43 | | 1.314 | | | | | | 10000 |
| I-44 | | 1.312 | | | | | | 10000 |
| I-45 | | 1.205 | | | | | | 10000 |
| I-46 | | 1.205 | | | | | | 10000 |
| I-47 | | 1.279 | | | | | | 10000 |
| I-48 | | 1.37 | | | | | | 10000 |
| I-49 | | 1.176 | | | | | | 10000 |
| I-50 | | 1.201 | | | | | | 10000 |
| I-51 | | 1.254 | | | | | | 10000 |
| I-52 | 3195 | 1.934 | 50 | | | | | 158 |
| I-53 | 5000 | 1.307 | | | | | | 1142 |
| I-54 | 3187 | 1.469 | | | | | | 482 |
| I-55 | 5000 | 1.351 | | | | | | 2384 |
| I-56 | | 2.164 | | | | | | 10000 |
| I-57 | | 2.152 | | | | | | 10000 |
| I-58 | | 1.495 | | | | | | 10000 |
| I-59 | | 1.531 | | | | | | 9876 |
| I-60 | | 1.365 | | | | | | 10000 |
| I-61 | | 1.446 | | | | | | 8905 |
| I-62 | | 0.981 | | | | | | 10000 |
| I-63 | | 1.019 | | | | | | 10000 |
| I-64 | 4246 | 1.222 | | | | | | 650 |
| I-65 | | 1.274 | | | | | | 2077 |
| I-66 | 1090 | 1.259 | | | | | | 174.5 |
| I-67 | 1227 | 1.24 | 50 | | | | | 154.5 |
| I-68 | | 1.553 | | | | | | 8120 |
| I-69 | | 1.593 | | | | | | 9096 |
| I-70 | | 1.609 | | | | | | 10000 |
| I-71 | | 1.686 | | | | | | 10000 |
| I-72 | | 1.721 | | | | | | 10000 |
| I-73 | | 1.723 | | | | | | 10000 |
| I-74 | | 1.329 | | | | | | 10000 |
| I-75 | | 1.376 | | | | | | 10000 |
| I-76 | | 1.198 | | | | | | 10000 |
| I-77 | | 1.285 | | | | | | 10000 |
| I-78 | 5000 | 1.242 | | | | | | 7478 |
| I-79 | | 1.274 | | | | | | 3970 |
| I-80 | | 1.045 | | | | | | 10000 |
| I-81 | | 1.063 | | | | | | 9775 |
| I-82 | | 1.003 | | | | | | 10000 |
| I-83 | | 1.032 | | | | | | 10000 |
| I-84 | | 0.985 | | | | | | 10000 |
| I-85 | | 0.998 | | | | | | 10000 |
| I-86 | | 1.237 | | | | | | 10000 |
| I-87 | | 1.152 | | | | | | 10000 |
| I-88 | | 1.239 | | | | | | 10000 |
| I-89 | | 0.967 | | | | | | 5640 |
| I-90 | | 0.958 | | | | | | 10000 |
| I-91 | | 1.13 | | | | | | 4720 |
| I-92 | | 1.199 | | | | | | 9155 |
| I-93 | | 1.321 | | | | | | 10000 |
| I-94 | | 1.409 | | | | | | 7009 |
| I-95 | | 1.308 | 33 | | | | | 8658 |
| I-96 | | 1.343 | | | | | | 10000 |
| I-97 | | 2.062 | | | | | | 7006.333 |
| I-98 | | 2.078 | | | | | | 10000 |
| I-99 | | 1.993 | | | | | | 10000 |
| I-100 | | 1.993 | | | | | | 9234 |
| I-101 | | 1.334 | | | | | | 10000 |
| I-102 | | 1.274 | | | | | | 10000 |
| I-103 | | 1.268 | | | | | | 4945 |
| I-104 | | 1.333 | | | | | | 6165 |
| I-105 | | 1.027 | | | | | | 10000 |
| I-106 | | 1.038 | | | | | | 10000 |
| I-107 | | 1.005 | | | | | | 10000 |
| I-108 | | 1.145 | | | | | | 10000 |
| I-109 | | 1.223 | | | | | | 8818 |
| I-110 | | 0.769 | | | | | | 8500 |
| I-111 | | 0.779 | | | | | | 7637 |
| I-112 | | 1.401 | | | | | | 10000 |
| I-113 | | 1.435 | | | | | | 6202 |
| I-114 | | 2.111 | | | | | | 4740 |
| I-115 | | 2.145 | | | | | | 4775 |
| I-116 | | 2.134 | 11 | | | | | 3639 |
| I-117 | | 1.328 | | | | | | 10000 |
| I-118 | | 1.545 | | | | | | 4363 |
| I-119 | | 1.17 | | | | | | 4943 |
| I-120 | | 1.243 | | | | | | 4553 |
| I-121 | | 0.825 | | | | | | 4462 |
| I-122 | | 0.84 | | | | | | 10000 |
| I-123 | 2112 | 1.978 | | | | | | 444 |
| I-124 | | 2.252 | | | | | | 1283 |
| I-125 | 3059 | 2.256 | | | | | | 569 |
| I-126 | | 2.239 | | | | | | 1478 |
| I-127 | 4841 | 2.245 | | | | | | 552 |
| I-128 | | 2.223 | | | | | | 907 |
| I-129 | 1981 | 2.108 | | | | | | 83 |
| I-130 | | 1.346 | | | | | | 797 |
| I-131 | 2160 | 1.496 | | | | | | 400 |
| I-132 | 5000 | 1.692 | 49 | | | | | 5094 |
| I-133 | 5000 | 2.383 | | | | | | 495 |
| I-134 | 2696 | 2.426 | | | | | | 99 |
| I-135 | 1867 | 2.178 | | | | | | 278.5 |
| I-136 | | 2.136 | | | | | | 3296 |
| I-137 | | 2.174 | | | | | | 1410 |
| I-138 | | 1.815 | | | | | | 10000 |
| I-139 | | 2.834 | | | | | | 10000 |
| I-140 | 2269 | 2.194 | 48 | | | | | 934 |
| I-141 | | 2.428 | | | | | | 5448 |
| I-142 | | 2.183 | | | | | | 7367 |
| I-143 | 2272 | 2.071 | | | | | | 733 |
| I-144 | 146.5 | 2.12 | | | | | | 66 |
| I-145 | | 2.283 | | | | | | 10000 |
| I-146 | | 2.004 | | | | | | 1224 |
| I-147 | 1637 | 1.399 | | | | | | 328 |
| I-148 | 2772 | 1.357 | | | | | | 398 |
| I-149 | | 1.27 | | | | | | 1789 |
| I-150 | | 1.297 | | | | | | 1940 |
| I-151 | | 1.355 | | | | | | 5502 |
| I-152 | 5000 | 1.337 | | | | | | 752 |
| I-153 | 2135 | 1.317 | | | | | | 153 |
| I-154 | 5000 | 1.426 | | | | | | 828 |
| I-155 | 5000 | 1.415 | | | | | | 734 |
| I-156 | | 1.066 | | | | | | 4177 |
| I-157 | | 1.202 | | | | | | 4059 |
| I-158 | | 1.23 | | | | | | 2170 |
| I-159 | | 1.452 | | | | | | 5353 |
| I-160 | | 1.49 | | | | | | 3238 |
| I-161 | | 0.816 | | | | | | 10000 |
| I-162 | | 0.827 | | | | | | 10000 |
| I-163 | | 1.29 | | | | | | 9752 |
| I-164 | | 1.359 | | | | | | 3613 |
| I-165 | | 1.998 | | | | | | 1233 |
| I-166 | 2164 | 2.06 | | | | | | 86.5 |
| I-167 | | 1.927 | | | | | | 6200 |
| I-168 | | 1.942 | | | | | | 5641 |
| I-169 | | 2.18 | | | | | | 9214 |
| I-170 | | 2.138 | | | | | | 4669 |
| I-171 | | 2.204 | | | | | | 2066 |
| I-172 | 5000 | 2.173 | 50 | | | | | 324 |
| I-173 | 3116 | 3.076 | 26 | | | | | 105 |
| I-174 | 1760 | 2.242 | | | | | | 116 |
| I-175 | 1760 | 2.242 | | | | | | 116 |
| I-176 | 1992 | 2.58 | | | | | | 549 |
| I-177 | 1992 | 2.58 | | | | | | 549 |
| I-178 | 1633 | 1.913 | | | | | | 152 |
| I-179 | 1633 | 1.913 | | | | | | 152 |
| I-180 | 561 | 2.193 | | | | | | 88 |
| I-181 | 310 | 2.213 | | | | | | 51 |
| I-182 | 2800 | 2.07 | | | | | | 750 |
| I-183 | | 2.213 | | | | | | 3766 |
| I-184 | 3109 | 2.265 | | | | | | 628 |
| I-185 | | 2.303 | | | | | | 2661 |
| I-186 | 5000 | 2.041 | | | | | | 518 |
| I-187 | 3318 | 2.083 | | | | | | 204 |
| I-188 | 1597 | 1.611 | | | | | | 156 |
| I-189 | 1597 | 1.611 | | | | | | 156 |
| I-190 | 696 | 1.736 | | | | | | 94 |
| I-191 | 1656 | 2.412 | | | | | | 240 |
| I-192 | 1656 | 2.412 | | | | | | 240 |
| I-193 | 533 | 2.462 | | | | | | 76 |
| I-194 | 1008 | 2.327 | | | | | | 105 |
| I-195 | | 2.983 | | | | | | 2177 |
| I-196 | | 3.711 | | | | | | 5513 |
| I-197 | | 3.809 | | | | | | 6145 |
| I-198 | 5000 | 2.368 | | | | | | 538 |
| I-199 | 5000 | 2.262 | | | | | | 796 |
| I-200 | 5000 | 2.278 | | | | | | 173 |
| I-201 | | 2.938 | | | | | | 6161 |
| I-202 | | 2.623 | | | | | | 7883 |
| I-203 | | 3.091 | | | | | | 5991 |
| I-204 | | 2.985 | | | | | | 7284 |
| I-205 | | 3.843 | | | | | | 10000 |
| I-206 | | 2.41 | | | | | | 2139 |
| I-207 | 1745 | 2.054 | | | | | | 143 |
| I-208 | 1745 | 2.054 | | | | | | 143 |
| I-209 | | 2.088 | | | | | | 1404 |
| I-210 | | 2.088 | | | | | | 1404 |
| I-211 | 5000 | 2.101 | | | | | | 367 |
| I-212 | 5000 | 2.101 | | | | | | 367 |
| I-213 | | 2.822 | | | | | | 8046 |
| I-214 | | 2.703 | | | | | | 7207 |
| I-215 | | 2.867 | | | | | | 10000 |
| I-216 | | 2.867 | | | | | | 10000 |
| I-217 | 1957 | 2.012 | | | | | | 90 |
| I-218 | 1957 | 2.012 | | | | | | 90 |
| I-219 | 2855 | 2.074 | | | | | | 131 |
| I-220 | 2855 | 2.074 | | | | | | 131 |
| I-221 | | 1.34 | | | | | | 6584 |
| I-222 | | 1.34 | | | | | | 6584 |
| I-223 | | 1.401 | | | | | | 4117 |
| I-224 | | 1.401 | | | | | | 4117 |
| I-225 | | 1.497 | | | | | | 7631 |
| I-226 | | 1.497 | | | | | | 7631 |
| I-227 | | 1.534 | 38 | | | | | 5912 |
| I-228 | | 1.534 | 38 | | | | | 5912 |
| I-229 | | 3.044 | | | | | | 5813 |
| I-230 | | 2.983 | | | | | | 4613 |
| I-231 | | 3.025 | | | | | | 6525 |
| I-232 | | 3.037 | | | | | | 7644 |
| I-233 | | 2.818 | | | | | | 5896 |
| I-234 | | 3.034 | | | | | | 7529 |
| I-235 | | 3.204 | | | | | | 7343 |
| I-236 | 5000 | 2.067 | | | | | | 384 |
| I-237 | 5000 | 2.067 | | | | | | 384 |
| I-238 | 1592 | 2.074 | | | | | | 267 |
| I-239 | 1592 | 2.074 | | | | | | 267 |
| I-240 | | 2.074 | | | | | | 3468 |
| I-241 | | 2.074 | | | | | | 3468 |
| I-242 | | 2.098 | | | | | | 4905 |
| I-243 | | 2.098 | | | | | | 4905 |
| I-244 | | 3.101 | | | | | | 2960 |
| I-245 | | 3.101 | | | | | | 2960 |
| I-246 | | 3.01 | | | | | | 3966 |
| I-247 | | 3.01 | | | | | | 3966 |
| I-248 | | 2.263 | | | | | | 2830 |
| I-249 | | 2.263 | | | | | | 2830 |
| I-250 | 5000 | 2.299 | | | | | | 1203 |
| I-251 | 5000 | 2.299 | | | | | | 1203 |
| I-252 | | 2.214 | | | | | | 3690 |
| I-253 | | 2.214 | | | | | | 3690 |
| I-254 | 5000 | 2.232 | | | | | | 1962 |
| I-255 | 5000 | 2.232 | | | | | | 1962 |
| I-256 | | 3.095 | 10 | | | | | 9207 |
| I-257 | | 2.913 | 10 | | | | | 3811 |
| I-258 | | 2.886 | | | | | | 2925 |
| I-259 | | 2.902 | | | | | | 4178 |
| I-260 | 5000 | 2.065 | | | | | | 683 |
| I-261 | 5000 | 2.014 | | | | | | 347 |
| I-262 | 5000 | 2.106 | | | | | | 967 |
| I-263 | 5000 | 2.128 | | | | | | 864 |
| I-264 | 5000 | 2.011 | | | | | | 364 |
| I-265 | 3762 | 2.032 | 36 | | | | | 194 |
| I-266 | 5000 | 2.016 | | | | | | 318 |
| I-267 | 5000 | 2.047 | 37 | | | | | 620 |
| I-268 | | 2.079 | 47 | | | | | 3999 |
| I-269 | | 1.814 | | | | | | 5311 |
| I-270 | 5000 | 2.496 | | | | | | 835 |
| I-271 | 5000 | 2.444 | 10 | | | | | 788 |
| I-272 | | 2.458 | | | | | | 1011 |
| I-273 | | 2.738 | 10 | | | | | 1890 |
| I-274 | | 2.619 | 10 | | | | | 2349 |
| I-275 | 5000 | 2.191 | 44 | | | | | 859 |
| I-276 | 3428 | 2.207 | | | | | | 329 |
| I-277 | | 2.186 | 45 | | | | | 6797 |
| I-278 | | 2.209 | | | | | | 2231 |
| I-279 | | 1.973 | | | | | | 4957 |
| I-280 | | 1.991 | | | | | | 3054 |
| I-281 | | 2.2 | | | | | | 6259 |
| I-282 | | 2.224 | | | | | | 3528 |
| I-283 | | 1.294 | | | | | | 9543 |
| I-284 | | 1.525 | 40 | | | | | 4348 |
| I-285 | 5000 | 1.491 | | | | | | 246 |
| I-286 | 4482 | 2.014 | | | | | | 450 |
| I-287 | 4954 | 1.875 | | | | | | 534 |
| I-288 | 1313 | 1.926 | 32 | | | | | 74 |
| I-289 | 5000 | 2.242 | | | | | | 626 |
| I-290 | 3286.5 | 2.271 | | | | | | 303 |
| I-291 | 3129 | 2.195 | | | | | | 327 |
| I-292 | 1871 | 2.208 | | | | | | 187 |
| I-293 | 139.5 | 2.382 | | | | | | 41 |
| I-294 | 130 | 1.996 | | | | | | 40 |
| I-295 | 5000 | 2.662 | | | | | | 1177 |
| I-296 | | 2.939 | | | | | | 1906 |
| I-297 | | 3.294 | | | | | | 5809 |
| I-298 | | 2.781 | | | | | | 3858 |
| I-299 | 1303 | 2.001 | | | | | | 189 |
| I-300 | | 2.817 | | | | | | |
| I-301 | | 3.262 | | | | | | |
| I-302 | | 2.38 | | | | | | |
| I-303 | | 3.131 | | | | | | |
| I-304 | 5000 | 2.339 | | | | | | |
| I-305 | 3982.5 | 2.348 | 10 | | | | | |
| I-306 | 5000 | 2.03 | | | | | | |
| I-307 | 4196 | 2.055 | | | | | | |
| I-308 | 5000 | 2.03 | | | | | | |
| I-309 | 3118 | 2.05 | | | | | | 181 |
| I-310 | | 2.188 | | | | | | 3243 |
| I-311 | | 2.226 | | | | | | 1238 |
| I-312 | | 2.134 | | | | | | 10000 |
| I-313 | | 2.159 | | | | | | 3600 |
| I-314 | | 2.344 | | | | | | 10000 |
| I-315 | | 2.359 | | | | | | 9897 |
| I-316 | 3982 | 1.906 | | | | | | 136 |
| I-317 | 5000 | 2.016 | | | | | | 443 |
| I-318 | 4633 | 2.001 | | | | | | 121 |
| I-319 | 1419 | 2.019 | | | | | | 54 |
| I-320 | | 2.041 | | | | | | 10000 |
| I-321 | | 2.073 | | | | | | 2242 |
| I-322 | 5000 | 1.933 | | | | | | 355 |
| I-323 | 3471 | 1.953 | | | | | | 92 |
| I-324 | | 2.596 | | | | | | 1745 |
| I-325 | 1310 | 1.976 | | | | | | 53 |
| I-326 | 1752 | 1.97 | 34 | | | | | |
| I-327 | 4724 | 1.958 | 43 | | | | | |
| I-328 | 1914 | 1.983 | 44 | | | | | |
| I-329 | 5000 | 2.01 | 41 | | | | | |
| I-330 | 5000 | 2.047 | 21 | | | | | |
| I-331 | 5000 | 2.024 | 41 | | | | | |
| I-332 | 5000 | 2.038 | 40 | | | | | |
| I-333 | 2756 | 2.12 | | | | | | |
| I-334 | 5000 | 1.863 | | | | | | |
| I-335 | 1544 | 1.928 | | | | | | |
| I-336 | 153 | 1.737 | 48 | | | | | |
| I-337 | 5000 | 1.579 | 48 | | | | | |
| I-338 | 5000 | 1.586 | 47 | | | | | |
| I-339 | 5000 | 1.328 | 50 | | | | | |
| I-340 | 5000 | 1.341 | 49 | | | | | |
| I-341 | 2000 | 1.732 | 38 | | | | | |
| I-342 | 58.5 | 2.074 | 50 | | | | | |
| I-343 | 172 | 1.931 | 50 | | | | | |
| I-344 | 42 | 1.977 | 44 | | | | | |
| I-345 | 134 | 2.144 | 49 | | | | | |
| I-346 | 180 | 1.943 | 44 | | | | | |
| I-347 | 121.5 | 1.956 | 50 | | | | | |
| I-348 | 5000 | 2.397 | 46 | | | | | 10000 |
| I-349 | 5000 | 2.415 | 49 | | | | | 7193 |
| I-350 | 5000 | 2.887 | 48 | | | | | 10000 |
| I-351 | 5000 | 2.944 | 50 | | | | | 10000 |
| I-352 | 5000 | 2.512 | 10 | | | | | 377 |
| I-353 | 5000 | 1.916 | 45 | | | | | 9291 |
| I-354 | 5000 | 1.947 | 50 | | | | | 2126 |
| I-355 | 5000 | 1.606 | 49 | | | | | 10000 |
| I-356 | 5000 | 1.619 | 50 | | | | | 8201 |
| I-357 | 5000 | 1.852 | 46 | −5.3 | | | | 10000 |
| I-358 | 5000 | 1.882 | 50 | | | | | 10000 |
| I-359 | 5000 | 2.055 | 30 | | | | | |
| I-360 | 145.5 | 2.017 | 44 | | | | | |
| I-361 | 42 | 2.058 | 46 | | | | | |
| I-362 | 3656 | 1.443 | 47 | | | | | |
| I-363 | 5000 | 2.09 | 32 | | | | | |
| I-364 | 5000 | 2.137 | 26 | | | | | |
| I-365 | 2715 | 2.038 | 48 | | | | | |
| I-366 | 1445 | 2.084 | | | | | | |
| I-367 | 177 | 2.154 | | | | | | |
| I-368 | 5000 | 2.168 | | | | | | |
| I-369 | 2311 | 1.661 | 40 | | | | | |
| I-370 | 1563 | 2.176 | 47 | | | | | |
| I-371 | 1389 | 2.136 | 48 | | | | | |
| I-372 | 2093 | 2.174 | 44 | | | | | |
| I-373 | 945 | 2.298 | 26 | | | | | |
| I-374 | 3011 | 2.242 | 49 | | | | | |
| I-375 | 3011 | 2.242 | 49 | | | | | |
| I-376 | 1321 | 2.296 | 48 | | | | | |
| I-377 | 1321 | 2.296 | 48 | | | | | |
| I-378 | 1336 | 2.143 | 50 | | | | | |
| I-379 | 1336 | 2.143 | 50 | | | | | |
| I-380 | 552 | 2.19 | 49 | | | | | |
| I-381 | 3653 | 2.636 | 10 | | | | | |
| I-382 | 2064 | 2.631 | 10 | | | | | |
| I-383 | 5000 | 1.859 | 36 | | | | | |
| I-384 | 177 | 1.837 | 45 | | | | | |
| I-385 | 3150 | 1.713 | 50 | | | | | |
| I-386 | 5000 | 2.14 | 47 | | | | | |
| I-387 | 2621 | 2.136 | 48 | | | | | |
| I-388 | 5000 | 2.863 | 10 | | | | | |
| I-389 | 5000 | 2.868 | 17 | | | | | |
| I-390 | 3422 | 2.587 | 10 | | | | | |
| I-391 | 1913 | 2.585 | 10 | | | | | |
| I-392 | 5000 | 2.518 | 10 | | | | | |
| I-393 | 1953 | 2.641 | 10 | | | | | |
| I-394 | 5000 | 3.86 | | | | | | |
| I-395 | 5000 | 4.073 | | | | | | |
| I-396 | 3427 | 2.819 | | | | | | |
| I-397 | 2229 | 2.828 | | | | | | |
| I-398 | 2155 | 1.846 | 46 | | | | | |
| I-399 | 2499 | 2.516 | | | | | | |
| I-400 | 3587 | 1.919 | | | | | | |
| I-401 | 134 | 2.281 | | | | | | |
| I-402 | 5000 | 3.393 | | | | | | |
| I-403 | 2128 | 2.865 | | | | | | |
| I-404 | 1851 | 2.396 | | | | | | |
| I-405 | 3821 | 1.723 | | | | | | |
| I-406 | 94 | 2.038 | 28 | | | | | |
| I-407 | 30 | 2.394 | 50 | 55.1 | | | | |
| I-408 | 120 | 2.674 | 50 | | | | | |
| I-409 | 70 | 2.741 | 48 | | | | | |
| I-410 | 50 | 2.26 | 50 | | | | | |
| I-411 | 189 | 2.567 | 50 | | | | | |
| I-412 | 92 | 2.651 | | | | | | |
| I-413 | 90 | 2 | 31 | | | | | |
| I-414 | 3417 | 2.096 | 19 | | | | | |
| I-415 | 2201 | 2.125 | 45 | | | | | |
| I-416 | 5000 | 2.058 | 44 | | | | | |
| I-417 | 5000 | 2.085 | 30 | | | | | |
| I-418 | 149 | 2.089 | 32 | | | | | |
| I-419 | 23 | 2.114 | | | | | | |
| I-420 | 127.5 | 2.157 | 24 | | | | | |
| I-421 | 99 | 2.087 | 36 | | | | | |
| I-422 | 27 | 2.136 | 24 | | | | | |
| I-423 | 105 | 2.089 | 48 | | | | | |
| I-424 | 170 | 1.968 | 50 | | | | | |
| I-425 | 50 | 2.01 | 45 | | | | | |
| I-426 | 109 | 1.959 | 33 | | | | | |
| I-427 | 38.5 | 1.985 | 50 | | | | | |
| I-428 | 60 | 1.987 | 14 | | | | | |
| I-429 | 146 | 2.069 | 50 | | | | | |
| I-430 | 154 | 2.133 | | | | | | |
| I-431 | 1320 | 2.225 | 33 | | | | | |
| I-432 | 145 | 2.234 | 23 | | | | | |
| I-433 | 136 | 2.078 | 39 | | | | | |
| I-434 | 67.5 | 2.049 | 20 | | | | | |
| I-435 | 70 | 2.133 | 50 | | | | | |
| I-436 | 56 | 2.159 | 50 | | | | | |
| I-437 | 26 | 2.001 | 44 | | | | | |
| I-438 | 5000 | 1.39 | 46 | | | | | |
| I-439 | 5000 | 1.408 | 50 | | | | | |
| I-440 | 5000 | 1.613 | 50 | | | | | |
| I-441 | 133 | 2.005 | 38 | | | | | |
| I-442 | 4484 | 2.098 | 40 | | | | | |
| I-443 | 2126 | 2.128 | 50 | | | | | |
| I-444 | 80 | 1.987 | 50 | | | | | |
| I-445 | 4962 | 2.054 | 43 | | | | | |
| I-446 | 130 | 1.97 | 44 | | | | | |
| I-447 | 74 | 1.996 | 50 | | | | | |
| I-448 | 92 | 1.976 | 50 | | | | | |
| I-449 | 53 | 1.998 | 30 | | | | | |
| I-450 | 3717 | 1.875 | 50 | | | | | |
| I-451 | 5000 | 1.88 | 50 | | | | | |
| I-452 | 1656 | 1.917 | 50 | | | | | |
| I-453 | 1554 | 2.007 | | | | | | |
| I-454 | 125 | 2.0462 | | | | | | |
| I-455 | 83.5 | 2.0374 | | | | | | |
| I-456 | 2168 | 2.0045 | | | | | | |
| I-457 | 1435 | 2.0242 | | | | | | |
| I-458 | 101.5 | 2.0703 | | | | | | |
| I-459 | 183 | 2.0572 | | | | | | |
| I-460 | 195.5 | 1.9935 | | | | | | |
| I-461 | 125 | 1.9364 | | | | | | |
| I-462 | 28 | 1.757 | 49 | | | | | |
| I-463 | 92 | 1.785 | | | | | | |
| I-464 | 57 | 1.831 | | | | | | |
| I-465 | 81 | 2.023 | 40 | | | | | |
| I-466 | 19 | 2.069 | 50 | | | | | |
| I-467 | 44 | 2.255 | 50 | | | | | |
| I-468 | 27 | 2.292 | 41 | | | | | |
| I-469 | 27 | 2.354 | | | | | | |
| I-470 | 11 | 2.394 | | 14.1 | | | | |
| I-471 | 52 | 2.429 | 13 | | | | | |
| I-472 | 62 | 2.416 | 36 | | | | | |
| I-473 | 121 | 2.076 | 39 | | | | | |
| I-474 | 43 | 2.114 | 50 | | | | | |
| I-475 | 102 | 2.277 | 43 | | | | | |
| I-476 | 48 | 2.317 | 49 | | | | | |
| I-477 | 119 | 2.005 | 49 | | | | | |
| I-478 | 71 | 2.052 | 42 | | | | | |
| I-479 | 27 | 2.094 | 38 | | | | | |
| I-480 | 176 | 2.096 | 36 | | | | | |
| I-481 | 42 | 2.133 | 30 | | | | | |
| I-482 | 118 | 2.485 | | | | | | |
| I-483 | 5000 | 1.845 | 43 | | | | | |
| I-484 | 5000 | 1.863 | 45 | | | | | |
| I-485 | 34 | 2.297 | 50 | | | | | |
| I-486 | 147 | 1.97 | 50 | | | | | |
| I-487 | 45 | 1.99 | 44 | | | | | |
| I-488 | 43 | 2.396 | 30 | | | | | |
| I-489 | 57 | 2.337 | 36 | | | | | |
| I-490 | 88 | 2.211 | 50 | | | | | |
| I-491 | 153 | 2.023 | 39 | | | | | |
| I-492 | 53 | 2.052 | 49 | | | | | |
| I-493 | 88 | 2.197 | 50 | | | | | |
| I-494 | 82 | 2.052 | 44 | | | | | |
| I-495 | 181 | 2.354 | | | | | | |
| I-496 | 5000 | 2.456 | 41 | | | | | |
| I-497 | 5000 | 2.6 | 29 | | | | | |
| I-498 | 5000 | 2.637 | 42 | | | | | |
| I-499 | 123 | 2.167 | 48 | | | | | |
| I-500 | 176 | 2.626 | 28 | | | | | |
| I-501 | 94 | 2.138 | 49 | | | | | |
| I-502 | 55 | 2.171 | 50 | | | | | |
| I-503 | 84 | 2.094 | 33 | | | | | |
| I-504 | 65 | 2.222 | 18 | | | | | |
| I-505 | 23 | 2.259 | 50 | | 5.6 | 4.9 | 10.8 | |
| I-506 | 25 | 2.412 | 10 | | | | | |
| I-507 | 192 | 2.079 | 47 | | | | | |
| I-508 | 73 | 2.126 | 50 | | | | | |
| I-509 | 99 | 2.163 | 41 | | | | | |
| I-510 | 128 | 2.057 | 43 | | | | | |
| I-511 | 40 | 2.099 | 29 | | | | | |
| I-512 | 20 | 2.437 | 17 | | | | | |
| I-513 | 143 | 1.569 | 50 | | | | | |
| I-514 | 30 | 1.911 | 50 | | | | | |
| I-515 | 54 | 2.028 | 46 | | | | | |
| I-516 | 23 | 2.072 | 42 | | | | | |
| I-517 | 15 | 2.421 | 15 | | | | | |
| I-518 | 143 | 1.966 | 39 | | | | | |
| I-519 | 33 | 2.004 | 50 | | | | | |
| I-520 | 45 | 2.313 | 50 | | | | | |
| I-521 | 23 | 2.351 | 50 | | | | | |
| I-522 | 75 | 2.214 | 50 | | | | | |
| I-523 | 44 | 2.241 | 50 | | | | | |
| I-524 | 2550 | 1.889 | 50 | | | | | |
| I-525 | 196 | 2.019 | | | | | | |
| I-526 | 56 | 2.049 | | | | | | |
| I-527 | 98 | 2.376 | | | | | | |
| I-528 | 63 | 2.395 | | | | | | |
| I-529 | 5000 | 2.854 | 50 | | | | | |
| I-530 | 5000 | 2.935 | 50 | | | | | |
| I-531 | 5000 | 2.794 | 43 | | | | | |
| I-532 | 5000 | 2.858 | 42 | | | | | |
| I-533 | 155 | 2.289 | 44 | | | | | |
| I-534 | 5000 | 2.432 | 31 | | | | | |
| I-535 | 5000 | 2.858 | 47 | | | | | |
| I-536 | 4469 | 2.746 | 23 | | | | | |
| I-537 | 5000 | 2.871 | 11 | | | | | |
| I-538 | 5000 | 2.753 | 33 | | | | | |
| I-539 | 5000 | 2.944 | 10 | | | | | |
| I-540 | 5000 | | | | | | | |
| I-541 | 180 | 2.087 | 50 | | | | | |
| I-542 | 109 | 2.135 | 39 | | | | | |
| I-543 | 32 | 2.474 | 28 | | | | | |
| I-544 | 24 | 2.478 | 48 | | | | | |
| I-545 | 21 | 2.574 | 46 | | | | | |
| I-546 | 16 | 2.4 | 50 | | | | | |
| I-547 | 43 | 2.45 | | | | | | |
| I-548 | 17 | 2.528 | 50 | | | | | |
| I-549 | 71 | 2.291 | | | | | | |
| I-550 | 30 | 2.343 | 50 | | | | | |
| I-551 | 45 | 2.278 | 50 | | | | | |
| I-552 | 15.5 | 2.334 | | | | | | |
| I-553 | 61 | 2.398 | | | | | | |
| I-554 | 27.5 | 2.457 | 35 | | | | | |
| I-555 | 41 | 2.374 | 41 | | | | | |
| I-556 | 20 | 2.435 | 39 | | | | | |
| I-557 | 35 | 2.391 | 47 | | | | | |
| I-558 | 56 | 2.308 | | | | | | |
| I-559 | 43 | 2.341 | | | | | | |
| I-560 | 81 | 2.391 | | | | | | |
| I-561 | 34 | 2.437 | 14 | | | | | |
| I-562 | 51 | 2.269 | | | | | | |
| I-563 | 19 | 2.317 | 50 | | | | | |
| I-564 | 36 | 2.374 | 47 | | | | | |
| I-565 | 13 | 2.435 | 26 | | | | | |
| I-566 | 76 | 2.175 | | | | | | |
| I-567 | 37 | 2.243 | 50 | 8.9 | | | | |
| I-568 | 73 | 2.158 | | | | | | |
| I-569 | 42 | 2.169 | 49 | | | | | |
| I-570 | 56 | 2.219 | | | | | | |
| I-571 | 32 | 2.245 | 24 | | | | | |
| I-572 | 73 | 2.064 | | | | | | |
| I-573 | 36 | 2.116 | 49 | | | | | |
| I-574 | 36 | 2.16 | 48 | | | | | |
| I-575 | 21 | 2.21 | 46 | | | | | |
| I-576 | 23 | 2.348 | 46 | | | | | |
| I-577 | 13 | 2.376 | 47 | | | | | |
| I-578 | 5000 | 2.257 | 50 | | | | | |
| I-579 | 5000 | 1.506 | 45 | | | | | |
| I-580 | 114 | 2.436 | 24 | | | | | |
| I-581 | 112 | 2.458 | | | | | | |
| I-582 | 17.5 | 2.504 | 22 | | | | | |
| I-583 | 69 | 2.563 | −1 | | | | | |
| I-584 | 33 | 2.635 | 21 | | | | | |
| I-585 | 5000 | 2.364 | 50 | | | | | |
| I-586 | 5000 | 2.39 | 50 | | | | | |
| I-587 | 5000 | 2.145 | 50 | | | | | |
| I-588 | 5000 | 2.873 | 49 | | | | | |
| I-589 | 48 | 2.119 | | | | | | |
| I-590 | 45 | 2.152 | | | | | | |
| I-591 | 64 | 2.348 | | | | | | |
| I-592 | 24 | 2.414 | | | | | | |
| I-593 | 68 | 2.119 | | | | | | |
| I-594 | 87 | 2.611 | | | | | | |
| I-595 | 57 | 2.644 | | | | | | |
| I-596 | 112 | 2.373 | | | | | | |
| I-597 | 76 | 2.39 | | | | | | |
| I-598 | 845 | 2.244 | 49 | | | | | |
| I-599 | 138 | 2.425 | 45 | | | | | |
| I-600 | 796 | 2.694 | 47 | | | | | |
| I-601 | 419 | 2.748 | 43 | | | | | |
| I-602 | 3150 | 3.146 | −1 | | | | | |
| I-603 | 392 | 2.624 | 10 | | | | | |
| I-604 | 692 | 2.609 | 10 | | | | | |
| I-605 | 632 | 2.617 | 10 | | | | | |
| I-606 | 289 | 2.412 | 49 | | | | | |
| I-607 | 253 | 2.416 | 48 | | | | | |
| I-608 | 211 | 2.506 | 40 | | | | | |
| I-609 | 80 | 2.534 | 43 | | | | | |
| I-610 | 54 | 2.587 | 49 | | | | | |
| I-611 | 55 | 2.595 | 48 | | | | | |
| I-612 | 64 | 2.414 | 49 | | | | | |
| I-613 | 1627 | 2.781 | 10 | | | | | |
| I-614 | 1143 | 2.801 | 10 | | | | | |
| I-615 | 50 | 2.826 | 10 | | | | | |
| I-616 | 108 | 2.413 | 10 | | | | | |
| I-617 | 253 | 2.549 | 10 | | | | | |
| I-618 | 135 | 2.617 | 10 | | | | | |
| I-619 | 44 | 2.677 | 47 | | | | | |
| I-620 | 35 | 2.688 | 48 | | 5.8 | | | |
| I-621 | 58 | 2.338 | 50 | | | | | |
| I-622 | 230 | 2.453 | 45 | | | | | |
| I-623 | 103 | 2.494 | 44 | | | | | |
| I-624 | 273 | 2.598 | 27 | | | | | |
| I-625 | 119 | 2.681 | 47 | | | | | |
| I-626 | 5000 | 2.048 | 50 | | | | | |
| I-627 | 338 | 3.002 | 47 | | | | | |
| I-628 | 713 | 3.103 | 47 | | | | | |
| I-629 | 685 | 3.109 | 40 | | | | | |
| I-630 | 5000 | 4.36 | | | | | | |
| I-631 | 5000 | 4.365 | | | | | | |
| I-632 | 5000 | 3.155 | | | | | | |
| I-633 | 88 | 2.624 | 10 | | | | | |
| I-634 | 425 | 2.764 | 49 | | | | | |
| I-635 | 171 | 2.734 | 43 | | | | | |
| I-636 | 198 | 2.569 | 50 | | | | | |
| I-637 | 5000 | 2.554 | 50 | | | | | |
| I-638 | 3876 | 2.622 | 48 | | | | | |
| I-639 | 109 | 2.49 | 44 | | | | | |
| I-640 | 87 | 2.516 | 50 | | | | | |
| I-641 | 122 | 2.497 | 43 | | | | | |
| I-642 | 118 | 2.512 | 48 | | | | | |
| I-643 | 958 | 2.833 | 28 | | | | | |
| I-644 | 808 | 2.837 | 22 | | | | | |
| I-645 | 395 | 2.677 | 40 | | | | | |
| I-646 | 461 | 2.694 | 41 | | | | | |
| I-647 | 445 | 2.677 | 50 | | | | | |
| I-648 | 233 | 2.721 | 46 | | | | | |
| I-649 | 209 | 2.677 | 41 | | | | | |
| I-650 | 137 | 2.718 | 45 | | | | | |
| I-651 | 186 | 2.167 | 48 | | | | | |
| I-652 | 72 | 2.233 | 48 | | | | | |
| I-653 | 108 | 2.07 | 39 | | | | | |
| I-654 | 20 | 2.121 | 47 | | | | | |
| I-655 | 183 | 2.688 | 46 | | | | | |
| I-656 | 73 | 2.736 | 40 | | | | | |
| I-657 | 46 | 2.767 | 40 | | | | | |
| I-658 | 546 | 2.859 | 48 | | | | | |
| I-659 | 430 | 2.903 | 48 | | | | | |
| I-660 | 544 | 2.754 | 45 | | | | | |
| I-661 | 45 | 2.099 | 45 | | | | | |
| I-662 | 26 | 2.125 | 46 | | | | | |
| I-663 | 43 | 1.798 | 42 | | | | | |
| I-664 | 996 | 2.433 | 45 | | | | | |
| I-665 | 923 | 2.448 | 47 | | | | | |
| I-666 | 626 | 2.862 | | | | | | |
| I-667 | 585 | 2.88 | | | | | | |
| I-668 | 675 | 2.199 | | | | | | |
| I-669 | 451 | 2.25 | | | | | | |
| I-670 | 5000 | 2.16 | | | | | | |
| I-671 | 3138 | 2.25 | | | | | | |
| I-672 | 5000 | 1.896 | | | | | | |
| I-673 | 5000 | 1.872 | | | | | | |
| I-674 | 5000 | 1.949 | | | | | | |
| I-675 | 4525 | 2.127 | | | | | | |
| I-676 | 5000 | 2.287 | | | | | | |
| I-677 | 22 | 2.449 | 49 | | | | | |
| I-678 | 26 | 2.329 | 49 | | | | | |
| I-679 | 7 | 2.382 | 48 | 44.8 | | | | |
| I-680 | 32 | 2.215 | 47 | | | | | |
| I-681 | 12 | 2.273 | 48 | | | | | |
| I-682 | 41 | 2.409 | 47 | | | | | |
| I-683 | 19 | 2.44 | 37 | | | | | |
| I-684 | 15 | 2.037 | 44 | | | | | |
| I-685 | 25 | 2.1 | 44 | | | | | |
| I-686 | 5 | 1.997 | 46 | 48.3 | 7.5 | 11.2, 8.5 | 5.6 | |
| I-687 | 12 | 2.035 | 38 | | | | | |
| I-688 | 6 | 1.924 | 46 | | | | | |
| I-689 | 11 | 1.957 | 50 | | | | | |
| I-690 | 7 | 2.013 | 44 | | | | | |
| I-691 | 9 | 2.068 | 45 | | | | | |
| I-692 | 9 | 2.12 | 38 | | | | | |
| I-693 | 12 | 2.184 | 34 | | | | | |
| I-694 | 5 | 2.073 | 40 | 32.7 | | | | |
| I-695 | 5 | 2.117 | | | | | | |
| I-696 | 5 | 1.997 | 40 | 17 | | | | |
| I-697 | 5 | 2.093 | 21 | | | | | |
| I-698 | 5 | 2.153 | 43 | | | | | |
| I-699 | 43 | 2.633 | 50 | | | | | |
| I-700 | 49 | 2.607 | | | | | | |
| I-701 | 34 | 2.631 | 46 | | | | | |
| I-702 | 76 | 2.747 | 40 | | | | | |
| I-703 | 57 | 2.762 | 50 | | | 11.9 | | |
| I-704 | 49 | 2.745 | 42 | | 7.9 | | | |
| I-705 | 58 | 2.887 | 25 | | | | | |
| I-706 | 57 | 2.987 | 48 | | | | | |
| I-707 | 46 | 2.963 | 48 | | | | | |
| I-708 | 52 | 2.976 | 47 | | 11.5 | | | |
| I-709 | 54 | 3.114 | 10 | | | | | |
| I-710 | 73 | 2.143 | 45 | | | | | |
| I-711 | 88 | 2.132 | 48 | | | | | |
| I-712 | 92 | 2.778 | 42 | | | | | |
| I-713 | 70 | 2.851 | 48 | | | | | |
| I-714 | 131 | 2.698 | 43 | | | | | |
| I-715 | 60.5 | 2.762 | 25 | | | | | |
| I-716 | 91 | 2.622 | 50 | | | | | |
| I-717 | 39 | 2.694 | 35 | | | | | |
| I-718 | 82 | 2.765 | 38 | | | | | |
| I-719 | 38 | 2.818 | 28 | | | | | |
| I-720 | 75 | 2.665 | 33 | | | | | |
| I-721 | 46 | 2.722 | 28 | | | | | |
| I-722 | 1128 | 2.518 | 27 | | | | | |
| I-723 | 3833 | 2.583 | 29 | | | | | |
| I-724 | 114 | 2.42 | 34 | | | | | |
| I-725 | 5000 | 2.609 | 42 | | | | | |
| I-726 | 5000 | 2.338 | 43 | | | | | |
| I-727 | 5000 | 2.414 | 26 | | | | | |
| I-728 | 289 | 2.354 | 38 | | | | | |
| I-729 | 56 | 2.366 | | | | | | |
| I-730 | 35 | 2.377 | 44 | | | | | |
| I-731 | 34 | 2.335 | 48 | | | | | |
| I-732 | 4219 | 2.121 | | | | | | |
| I-733 | 4261 | 2.154 | 43 | | | | | |
| I-734 | 370 | 2.713 | | | | | | |
| I-735 | 246 | 2.738 | 43 | | | | | |
| I-736 | 440 | 2.722 | | | | | | |
| I-737 | 353 | 2.729 | 44 | | | | | |
| I-738 | 63 | 2.455 | 41 | | | | | |
| I-739 | 53 | 2.406 | 10 | | | | | |
| I-740 | 376 | 2.822 | | | | | | |
| I-741 | 226 | 2.915 | 10 | | | | | |
| I-742 | 159 | 2.804 | 27 | | | | | |
| I-743 | 85 | 2.604 | 29 | | | | | |
| I-744 | 112 | 2.557 | | | | | | |
| I-745 | 80 | 2.61 | 10 | | | | | |
| I-746 | 5000 | 2.372 | | | | | | |
| I-747 | 2388 | 2.492 | 15 | | | | | |
| I-748 | 54 | 2.459 | | | | | | |
| I-749 | 40 | 2.472 | 45 | | | | | |
| I-750 | 43 | 2.441 | 48 | | | | | |
| I-751 | 29 | 2.513 | 48 | | | | | |
| I-752 | 16 | 2.419 | | 53 | | 9.9 | | |
| I-753 | 14 | 2.432 | 50 | | | 10.0 | | |
| I-754 | 115 | 2.666 | | | | | | |
| I-755 | 88 | 2.684 | 47 | | | | | |
| I-756 | 56 | 2.606 | 49 | | | | | |
| I-757 | 64 | 2.546 | | | | | | |
| I-758 | 47 | 2.571 | 47 | | | | | |
| I-759 | 1534 | 2.582 | | | | | | |
| I-760 | 608 | 2.66 | −1 | | | | | |
| I-761 | 5 | 2.207 | 42 | 63.4 | 5.0 | | | |
| I-762 | 6 | 2.247 | 45 | | | 10.0 | 6.0 | |
| I-763 | 5 | 1.967 | | | | | | |
| I-764 | 14 | 2.212 | 45 | | | | | |
| I-765 | 228 | 1.71 | 49 | | | | | |
| I-766 | 55 | 1.853 | 50 | | | | | |
| I-767 | 12 | 2.004 | 50 | | | | | |
| I-768 | 9 | 2.043 | | | | | | |
| I-769 | 5 | 2.307 | | | | 13.0 | | |
| I-770 | 5 | 2.358 | | | | 13.7 | | |
| I-771 | 5 | 2.031 | | | | | | |
| I-772 | 7 | 2.069 | | | | | | |
| I-773 | 167 | 1.76 | 47 | | | | | |
| I-774 | 38 | 1.928 | 46 | | | | | |
| I-775 | 7 | 2.092 | 47 | 5.1 | | | | |
| I-776 | 5 | 2.137 | 41 | | | | | |
| I-777 | 5 | 2.155 | 50 | 24.5 | 14.4 | | | |
| I-778 | 8 | 2.035 | 43 | | | | | |
| I-779 | 5 | 2.124 | 44 | | | | | |
| I-780 | 9 | 2.081 | 50 | | 17.1 | | | |
| I-781 | 5 | 2.163 | 41 | 24 | 11.6 | | | |
| I-782 | 6 | 2.212 | 38 | | | | | |
| I-783 | 7 | 2.19 | 48 | 50.9 | 7.5 | | | |
| I-784 | 6 | 2.29 | 21 | 35.1 | 12.1 | | | |
| I-785 | 6 | 2.278 | 42 | 31 | | | | |
| I-786 | 8 | 2.298 | 39 | | 17.1 | | | |
| I-787 | 5 | 2.077 | 50 | | | | | |
| I-788 | 5 | 2.173 | 31 | | | | | |
| I-789 | 19 | 2.082 | 50 | 9.7 | | | | |
| I-790 | 24 | 2.125 | 50 | | | | | |
| I-791 | 310 | 2.054 | 48 | | | | | |
| I-792 | 20 | 1.963 | 33 | | | | | |
| I-793 | 14 | 2.387 | 10 | | | | | |
| I-794 | 19 | 2.378 | 40 | | | | | |
| I-795 | 176 | 2.387 | 10 | | | | | |
| I-796 | 8143 | 2.447 | 35 | | | | | |
| I-797 | 10000 | 2.321 | 45 | 4.3 | >20 | | | |
| I-798 | 106 | 2.068 | | | | | | |
| I-799 | 92 | 2.057 | | 0.9 | | | | |
| I-800 | 79 | 1.997 | | 8.1 | | | | |
| I-801 | 6 | 2.205 | 50 | | 6.8 | | | |
| I-802 | 6 | 2.301 | 10 | | | | | |
| I-803 | 5 | 2.03 | 40 | | 9.0 | | | |
| I-804 | 5 | 2.112 | 44 | | | | | |
| I-805 | 5 | 1.979 | 50 | | | | | |
| I-806 | 5 | 2.039 | 37 | | | | | |
| I-807 | 5 | 2.265 | 24 | | 5.3 | | 3.4 | |
| I-808 | 5 | 2.343 | 10 | | | | | |
| I-809 | 5 | 2.097 | 36 | | 11.7 | | | |
| I-810 | 6 | 2.165 | 39 | | | | | |
| I-811 | 32 | 1.684 | 43 | | | | | |
| I-812 | 33 | 1.897 | 47 | | | | | |
| I-813 | 7 | 2.166 | 28 | | | | | |
| I-814 | 7 | 2.096 | 47 | | 12.7 | | | |
| I-815 | 5 | 2.096 | 46 | | 11.8 | | | |
| I-816 | 5 | 2.052 | 46 | | | | | |
| I-817 | 10 | 2.233 | 47 | 45 | 6.8 | | | |
| I-818 | 6 | 2.117 | 48 | 31.4 | | | | |
| I-819 | 5 | 2.028 | 50 | 37.8 | | | | |
| I-820 | 6 | 2.095 | 38 | 30.8 | | | | |
| I-821 | 5 | 2.068 | 36 | | | | | |
| I-822 | 6 | 2.026 | 46 | 53 | 7.7 | | | |
| I-823 | 5 | 2.086 | 32 | 37.5 | >10 | | | |
| I-824 | 7 | 2.06 | 36 | | | | | |
| I-825 | 15 | 2.028 | | | | | | |
| I-826 | 15 | 2.066 | 30 | | | | | |
| I-827 | 13 | 2.1 | | | | | | |
| I-828 | 13 | 2.137 | 40 | | | | | |
| I-829 | 15 | 2.084 | 34 | | | | | |
| I-830 | 15 | 2.042 | | | | | | |
| I-831 | 11 | 2.075 | 50 | 32.4 | | | | |
| I-832 | 9 | 2.095 | | | | | | |
| I-833 | 8 | 2.146 | 50 | | | | | |
| I-834 | 14 | 2.08 | | | | | | |
| I-835 | 11 | 2.109 | 50 | 27.6 | | | | |
| I-836 | 11 | 2.22 | 41 | | | 16.9 | | |
| I-837 | 5 | 2.298 | 14 | | | 23.8 | | |
| I-838 | 12 | 2.186 | 41 | | | 17.4 | | |
| I-839 | 7 | 2.265 | 13 | | | 41.9 | | |
| I-840 | 16 | 2.191 | 32 | | | 22.3 | | |
| I-841 | 12 | 2.258 | 14 | | | 34.4 | | |
| I-842 | 34 | 2.392 | 10 | | | 47.9 | | |
| I-843 | 154 | 2.489 | 10 | | | | | |
| I-844 | 6 | 2.305 | 35 | | 10.7 | | | |
| I-845 | 7 | 2.417 | 10 | | | | | |
| I-846 | 35 | 2.415 | 18 | 4.3 | | | | |
| I-847 | 28 | 2.511 | 10 | | | | | |
| I-848 | 13 | 2.464 | | | | 7.0 | | |
| I-849 | 7 | 2.393 | | | 2.3 | 2.6 | | |
| I-850 | 26 | 2.261 | | | | 16.4 | | |
| I-851 | 16 | 2.147 | | | | 7.6 | | |
| I-852 | 7 | 2.397 | | | | 2.8 | | |
| I-853 | 8 | 2.515 | | | 6.6 | | | |
| I-854 | 8 | 2.48 | | | 4.6 | 3.6 | | |
| I-855 | 22 | 2.535 | | | | | | |
| I-856 | 17 | 2.453 | | | | | | |
| I-857 | 15 | 2.581 | 10 | | 50.6 | | | |
| I-858 | 34 | 2.543 | 18 | | | | | |
| I-859 | 36 | 2.711 | | | | | | |
| I-860 | 61 | 2.722 | 10 | | | | | |
| I-861 | 40 | 2.548 | 20 | | | | | |
| I-862 | 123 | 2.85 | 10 | | | | | |
| I-863 | 22 | 2.284 | 10 | | | | | |
| I-864 | 12 | 2.622 | 10 | | 29.1 | | | |
| I-865 | | 1.62 | 38 | | | | | |
| I-866 | | | 50 | | | | | |
| I-867 | | 1.802 | 13 | | | | | |
| I-868 | | 1.882 | 10 | | | | | |
| I-869 | | 1.912 | 10 | | | | | |
| I-870 | 9.5 | 1.99 | 47 | | | | | |
| I-871 | 5 | 1.917 | 45 | | | | | |
| I-872 | 9.5 | 1.893 | 50 | | | | | |
| I-873 | 6.5 | 1.995 | 47 | | 8.9 | | | |
| I-874 | 18.5 | 2.315 | 50 | | | | | |
| I-875 | 26 | 2.208 | −1 | | | | | |
| I-876 | 24 | 2.257 | 50 | | | | | |
| I-877 | 11 | 2.346 | 50 | | | | | |
| I-878 | 6 | 2.093 | 46 | | | | | |
| I-879 | 6 | 1.998 | 45 | | | | | |
| I-880 | 6 | 1.722 | 42 | | | | | |
| I-881 | 10 | −0.008 | — | | | | | |
| I-882 | 22 | 2.028 | — | | 22.5 | | | |
| I-883 | 53 | 2.397 | 42 | | | | | |
| I-884 | 14 | 2.112 | 34 | | | | | |
| I-885 | 32 | 2.39 | 43 | | | | | |
| I-886 | 22 | 1.506 | 43 | | | | | |
| I-887 | 14 | 1.373 | 46 | | | | | |
| I-888 | 13 | 1.391 | 21 | | | | | |
| I-889 | 16 | 1.486 | 41 | | | | | |
| I-890 | 16 | 1.668 | 40 | | | | | |
| I-891 | 13 | 1.526 | 42 | | | | | |
| I-892 | 18 | 1.475 | 33 | | | | | |
| I-893 | 834 | 1.759 | −1 | | | | | |
| I-894 | 15 | 1.451 | −1 | | | | | |
| I-895 | 14 | 1.448 | 31 | | | | | |
| I-896 | 22 | 1.446 | 30 | | | | | |
| I-897 | 16 | 1.653 | 43 | | | | | |
| I-898 | 16 | 2.201 | 42 | | | | | |
| I-899 | 28 | 2.263 | 40 | | | | | |
| I-900 | 33 | 2.743 | 41 | | | | | |
| I-901 | 38 | 2.492 | 40 | | | | | |
| I-902 | 104 | 2.688 | −1 | | | | | |
| I-903 | 33 | 2.383 | 43 | | | | | |
| I-904 | 54 | 2.646 | 30 | | | | | |
| I-905 | 8 | 1.921 | 46 | | | | | |
| I-906 | 8 | 1.75 | 46 | | >20 | | | |
| I-907 | 9 | 1.802 | 20 | | | | | |
| I-908 | 10 | 1.819 | 44 | | | | | |
| I-909 | 8 | 1.926 | 44 | | | | | |
| I-910 | 10 | 1.841 | 40 | | | | | |
| I-911 | 12 | 1.822 | −1 | | | | | |
| I-912 | 10 | 1.826 | 45 | | | | | |
| I-913 | 10 | 1.824 | 49 | | | | | |
| I-914 | 16 | 1.819 | 46 | | | | | |
| I-915 | 28 | 2.326 | 41 | | | | | |
| I-916 | 28 | 2.328 | 45 | | | | | |
| I-917 | 12 | 2.152 | 44 | | | | | |
| I-918 | 33 | 2.494 | 44 | | | | | |
| I-919 | 28 | 2.55 | 44 | | | | | |
| I-920 | 117 | 3.076 | 50 | | | | | |
| I-921 | 8 | 2.262 | 24 | | 4.0 | | | |
| I-922 | 12 | 2.344 | 32 | | 2.3 | 2.7 | | |
| I-923 | 27 | 2.462 | — | | | 6.0 | | |
| I-924 | 7 | 2.277 | — | | | 3.3 | 4.5 | |
| I-925 | 21 | 2.344 | — | | | | | |
| I-926 | 43 | 2.406 | — | | 17.1 | | | |
| I-927 | 11 | 2.313 | — | | | 8.2 | 4.2 | |
| I-928 | 21 | 2.38 | — | | 5.0 | | | |
| I-929 | 23 | 2.74 | — | | 7.0 | | | |
| I-930 | 37 | 2.962 | — | | 5.6 | | | |
| I-931 | 221 | 3.358 | — | | | | | |
| I-932 | 35 | 2.769 | — | | | 11.4 | | |
| I-933 | 168 | 3.026 | — | | >20 | | | |
| I-934 | 69 | 2.817 | — | | | | | |
| I-935 | 8 | 1.928 | — | | | | | |
| I-936 | 7 | 1.935 | — | | | | | |
| I-937 | 9 | 1.937 | — | | | | | |
| I-938 | 9 | 2.162 | — | | | | | |
| I-939 | 8 | 1.786 | 15 | | 18.0 | | | |
| I-940 | 1742 | 2.055 | 45 | | | | | |
| I-941 | 2111 | 2.011 | 46 | | | | | |
| I-942 | 1285 | 1.984 | 46 | | | | | |
| I-943 | 1994 | 2.071 | 49 | | | | | |
| I-944 | 761 | 1.982 | −1 | | | | | |
| I-945 | 1079 | 2.104 | −1 | | | | | |
| I-946 | 798 | 2.209 | 47 | | | | | |
| I-947 | 1053 | 2.271 | 47 | | | | | |
| I-948 | 2433 | 2.013 | 47 | | | | | |
| I-949 | 2256 | 2.086 | 49 | | | | | |
| I-950 | 1634 | 1.9 | 46 | | | | | |
| I-951 | 1471 | 1.971 | 47 | | | | | |
| I-952 | 1214 | 1.975 | −1 | | | | | |
| I-953 | 1456 | 2.04 | 47 | | | | | |
| I-954 | 900 | 1.918 | 46 | | | | | |
| I-955 | 1198 | 1.998 | 45 | | | | | |
| I-956 | 1086 | 1.875 | 46 | | | | | |
| I-957 | 1426 | −0.043 | 46 | | | | | |
| I-958 | 606 | 1.417 | 47 | | | | | |
| I-959 | 881 | −0.036 | 48 | | | | | |
| I-960 | 336 | 2.06 | 13 | | | | | |
| I-961 | 357 | 2.123 | 10 | | | | | |
| I-962 | 407 | 2.046 | −1 | | | | | |
| I-963 | 402 | 2.117 | −1 | | | | | |
| I-964 | 1435 | 1.802 | 46 | | | | | |
| I-965 | 1780 | 1.903 | 45 | | | | | |
| I-966 | 2023 | 1.791 | 43 | | | | | |
| I-967 | 1735 | 1.884 | 45 | | | | | |
| I-968 | 2981 | 1.752 | 30 | | | | | |
| I-969 | 2318 | 1.851 | 46 | | | | | |
| I-970 | 1364 | 1.853 | −1 | | | | | |
| I-971 | 1607 | 1.939 | 10 | | | | | |
| I-972 | 1819 | 1.879 | 30 | | | | | |
| I-973 | 1377 | 1.95 | 10 | | | | | |
| I-974 | 1780 | 1.857 | 10 | | | | | |
| I-975 | 1706 | 1.952 | 46 | | | | | |
| I-976 | 399 | 1.78 | 50 | | | | | |
| I-977 | 367 | 1.87 | 46 | | | | | |
| I-978 | 112 | 1.745 | −1 | | | | | |
| I-979 | 240 | 1.809 | −1 | | | | | |
| I-980 | 221 | 1.695 | 19 | | | | | |
| I-981 | 199 | 1.761 | 45 | | | | | |
| I-982 | 190 | 1.853 | 16 | | | | | |
| I-983 | 118 | 1.919 | 10 | | | | | |
| I-984 | 179 | 1.8 | 45 | | | | | |
| I-985 | 301 | 1.87 | 47 | | | | | |
| I-986 | 136 | 1.855 | 27 | | | | | |
| I-987 | 112 | 2.115 | 10 | | | | | |
| I-988 | 5 | 2.345 | 11 | | | 13.9 | 5.2 | |
| I-989 | 87 | 2.1 | 10 | | | | | |
| I-990 | 41 | 2.647 | 10 | | | 8.8 | | |
| I-991 | 51 | 2.696 | 10 | | | | | |
| I-992 | 59 | 2.561 | 12 | | | | | |
| I-993 | 12 | 2.334 | 41 | | | 4.0 | 4.4 | |
| I-994 | 121 | 3.205 | 10 | | | | | |
| I-995 | 55 | 2.799 | 34 | | | | | |
| I-996 | 21 | 2.839 | 48 | | | 11.3 | 10.8 | |
| I-997 | 154 | 3.415 | 10 | | | | | |
| I-998 | 127 | 2.87 | 43 | | | | | |
| I-999 | 26 | 2.941 | 30 | | | 26.3 | 11.1 | |
| I-1000 | 18 | 2.967 | 44 | | | 23.9 | 8.0 | |
| I-1001 | 5526 | 1.886 | −1 | | | >>20 | >>20 | |
| I-1002 | 9 | 2.605 | −1 | | | 29.8 | >>20 | |
| I-1003 | 38 | 2.543 | 10 | | | | | |
| I-1004 | 5 | 1.996 | 47 | | | 10.6 | >20 | |
| I-1005 | 42 | 2.641 | 10 | | | | | |
| I-1006 | 5 | 1.703 | 30 | | | >20 | | |
| I-1007 | 6 | 2.449 | 18 | | | 24.9 | 3.3 | |
| I-1008 | 9 | 2.431 | 34 | | | 14.0 | 13.0 | |
| I-1009 | 11 | 2.038 | −1 | | | 33.3 | | |
| I-1010 | 8 | 2.356 | −1 | | | 13.3 | | |
| I-1011 | 5 | 2.034 | 23 | | | 20.8 | >20 | |
| I-1012 | 60 | 2.61 | 10 | | | | | |
| I-1013 | 8 | 2.133 | 47 | | | | | |
| I-1014 | 19 | 2.38 | 10 | | | | | |
| I-1015 | 5 | 2.27 | 47 | | | 8.9 | 9.0 | |
| I-1016 | 8 | 2.38 | 10 | | | 21.4 | 21.4 | |
| I-1017 | 5 | 2.113 | 48 | | | 8.9 | >20 | |
| I-1018 | 26 | 2.347 | 10 | | | | | |
| I-1019 | 5 | 2.219 | 48 | | | 9.6 | 8.3 | |
| I-1020 | 52 | 2.488 | 10 | | | | | |
| I-1021 | 5 | 2.049 | 44 | | | 11.5 | | |
| I-1022 | 33 | 2.285 | 20 | | | | | |
| I-1023 | 10 | 2.175 | 46 | | | | | |
| I-1024 | 43 | 2.449 | 14 | | | | | |
| I-1025 | 5 | | | | | 23.6 | >20 | |
| I-1026 | 5 | | | | | 16.7 | >20 | |
| I-1027 | 1140 | | | | | | | |
| I-1028 | 13 | 2.402 | 10 | | | 8.7 | 6.4 | |
| I-1029 | 823 | 2.36 | 49 | | | >20 | >20 | |
| I-1030 | 507 | 2.757 | 47 | | | >20 | >20 | |
| I-1031 | 11 | 2.42 | 47 | | | 8.8 | | |
| I-1032 | 15 | 2.431 | 44 | | | | | |
| I-1033 | 10 | 2.504 | 34 | | | | | |
| I-1034 | 27 | 2.709 | 12 | | | 9.5 | 11.9 | |
| I-1035 | 21 | 2.629 | 28 | | | 10.2 | >20 | |
| I-1036 | 29 | 2.46 | −1 | | | | | |
| I-1037 | 30 | 2.521 | −1 | | | | | |
| I-1038 | 101 | 2.729 | 10 | | | | | |
| I-1039 | 57 | 2.654 | 20 | | | | | |
| I-1040 | 8 | 2.268 | 50 | | | 9.3 | | |
| I-1041 | 8 | 2.084 | 50 | | | >>20 | | |
| I-1042 | 5 | 2.354 | 46 | | | | | |
| I-1043 | 12 | 2.148 | 43 | | | | | |
| I-1044 | 5 | 2.318 | 43 | | | 17.6 | 20.0 | |
| I-1045 | 5 | 2.117 | 49 | | | 23.9 | | |
| I-1046 | 5 | 2.398 | 37 | | | 20.5 | | |
| I-1047 | 5 | 2.193 | 10 | | | 28.4 | | |
| I-1048 | 5 | 2.517 | 42 | | | 8.8 | 10.5 | |
| I-1049 | 6 | 2.285 | 48 | | | | | |
| I-1050 | 5 | 2.446 | 43 | | | 24.8 | 11.2 | |
| I-1051 | 14 | 2.228 | 48 | | | >>20 | >>20 | |
| I-1052 | 9 | 2.334 | 33 | | | | | |
| I-1053 | 11 | 2.381 | 48 | | | >20 | | |
| I-1054 | 5 | 2.186 | 46 | | | >20 | | |
| I-1055 | 7 | 2.113 | 10 | | | | | |
| I-1056 | 6494 | 1.929 | 41 | | | | | |
| I-1057 | 6641 | 1.92 | 50 | | | | | |
| I-1058 | 14 | 2.249 | 42 | | | | | |
| I-1059 | 42 | 2.444 | 42 | | | | | |
| I-1060 | 38 | 2.602 | 10 | | | | | |
| I-1061 | 6 | 2.562 | 10 | | | 10.3 | | |
| I-1062 | 393 | 1.898 | −1 | | | | | |
| I-1063 | 471 | 1.644 | 43 | | | | | |
| I-1064 | 414 | 1.967 | 29 | | | | | |
| I-1065 | 520 | 2.044 | −1 | | | | | |
| I-1066 | 789 | 2.024 | 10 | | | | | |
| I-1067 | 517 | 2.147 | −1 | | | | | |
| I-1068 | 950 | 2.053 | 13 | | | | | |
| I-1069 | 920 | 2.113 | 10 | | | | | |
| I-1070 | 1022 | 2.135 | 10 | | | | | |
| I-1071 | 1261 | 2.218 | 33 | | | | | |
| I-1072 | 723 | 1.389 | 35 | | | | | |
| I-1073 | 409 | 1.831 | 31 | | | | | |
| I-1074 | 498 | 1.92 | 34 | | | | | |
| I-1075 | 567 | 1.971 | 17 | | | | | |
| I-1076 | 448 | 2.049 | −1 | | | | | |
| I-1077 | 324 | 2.06 | 10 | | | | | |
| I-1078 | 352 | 2.162 | 27 | | | | | |
| I-1079 | 5 | 2.498 | 45 | | | 19.7 | 14.5 | |
| I-1080 | 6 | 2.593 | 47 | | | >20 | 17.2 | |
| I-1081 | 5 | 2.398 | 43 | | | 16.7 | 10.0 | |
| I-1082 | 5 | 2.331 | 48 | | | 14.1 | 4.8 | |
| I-1083 | 8 | | | | | 18.8 | | |
| I-1084 | 368 | | | | | | | |
| I-1085 | 390 | 3.702 | 10 | | | | | |
| I-1086 | 12 | 2.709 | −1 | | | | | |
| I-1087 | 9 | 2.766 | 44 | | | 9.3 | >20 | |
| I-1088 | 22 | 2.8 | 44 | | | | | |
| I-1089 | 12 | 2.826 | 43 | | | | | |
| I-1090 | 18 | 2.982 | 39 | | | | | |
| I-1091 | 27 | 3.042 | 33 | | | | | |
| I-1092 | 26 | 3.173 | 28 | | | | | |
| I-1093 | 43 | 3.22 | 48 | | | | | |
| I-1094 | 46 | 3.271 | −1 | | | | | |
| I-1095 | 15 | 3.089 | −1 | | | >20 | >20 | |
| I-1096 | 17 | 3.18 | 10 | | | | | |
| I-1097 | 40 | 3.611 | 20 | | | >20 | >20 | |
| I-1098 | 100 | 3.538 | 10 | | | | | |
| I-1099 | 9 | 2.16 | 47 | | | 9.0 | | |
| I-1100 | 16 | 2.357 | 46 | | | | | |
| I-1101 | 14 | 2.216 | 48 | | | | | |
| I-1102 | 16 | 2.394 | 10 | | | | | |
| I-1103 | 15 | 2.18 | 46 | | | | | |
| I-1104 | 10 | 2.383 | 48 | | | | | |
| I-1105 | 36 | 2.328 | 48 | | | >20 | | |
| I-1106 | 23 | 2.439 | 47 | | | >20 | | |
| I-1107 | 18 | 2.637 | 10 | | | | | |
| I-1108 | 18 | 2.288 | 42 | | | | | |
| I-1109 | 24 | 2.308 | 45 | | | | | |
| I-1110 | 38 | 2.37 | 10 | | | | | |
| I-1111 | 28 | 2.357 | 10 | | | | | |
| I-1112 | 40 | 2.719 | 10 | | | | | |
| I-1113 | 64 | 2.986 | 10 | | | | | |
| I-1114 | 20 | 2.492 | 28 | | | | | |
| I-1115 | 28 | 2.503 | 32 | | | | | |
| I-1116 | 40 | 2.62 | 13 | | | | | |
| I-1117 | 55 | 2.611 | 10 | | | | | |
| I-1118 | 24 | 2.341 | 37 | | | | | |
| I-1119 | 26 | 2.507 | 31 | | | | | |
| I-1120 | 85 | 2.544 | 41 | | | | | |
| I-1121 | 18 | 2.306 | 41 | | | | | |
| I-1122 | 34 | 2.412 | 14 | | | | | |
| I-1123 | 64 | 2.465 | 12 | | | | | |
| I-1124 | 12 | 2.222 | 47 | | | >20 | | |
| I-1125 | 16 | 2.394 | 45 | | | >20 | | |
| I-1126 | 32 | 2.419 | 49 | | | | | |
| I-1127 | 18 | 2.183 | 47 | | | | | |
| I-1128 | 20 | 2.297 | 23 | | | | | |
| I-1129 | 39 | 2.335 | 29 | | | | | |
| I-1130 | 6 | | 15 | | | 7.6 | | |
| I-1131 | 7 | | 39 | | | 18.8 | | |
| I-1132 | 21 | | 49 | | | >>20 | | |
| I-1133 | 5 | | −1 | | | | | |
| I-1134 | 6 | | | | | | | |
| I-1135 | 20 | | 39 | | | | | |
| I-1136 | 16 | | 40 | | | 14.9 | | |
| I-1137 | 8 | | −1 | | | 12.0 | | |
| I-1138 | 18 | | −1 | | | 19.8 | | |
| I-1139 | 22 | | −1 | | | 18.0 | | |
| I-1140 | 33 | | 10 | | | | | |
| I-1141 | 13 | | 44 | | | | | |
| I-1142 | 22 | | 46 | | | | | |
| I-1143 | 12 | | 44 | | | | | |
| I-1144 | 28 | | 32 | | | | | |
| I-1145 | 18 | | 10 | | | | | |
| I-1146 | 33 | | 18 | | | | | |
| I-1147 | 10 | | 23 | | | | | |
| I-1148 | 50 | | 10 | | | | | |
| I-1149 | 70 | | 10 | | | | | |
| I-1150 | 12 | | 18 | | | | | |
| I-1151 | 30 | | 10 | | | | | |
| I-1152 | 65 | | 10 | | | | | |
| I-1153 | 12 | | −1 | | | | | |
| I-1154 | 45 | | −1 | | | | | |
| I-1155 | 60 | | −1 | | | | | |
| I-1156 | 14 | | 48 | | | | | |
| I-1157 | 36 | | 15 | | | | | |
| I-1158 | 168 | | 29 | | | | | |
| I-1159 | 61 | 1.876 | 45 | | | | | |
| I-1160 | 575 | 1.406 | −1 | | | | | |
| I-1161 | 24 | 2.076 | −1 | | | >20 | | |
| I-1162 | 1534 | | −1 | | | | | |
| I-1163 | 691 | 2.059 | 48 | | | | | |
| I-1164 | 162 | 2.13 | 50 | | | | | |
| I-1165 | 722 | | −1 | | | | | |
| I-1166 | 14 | | 14 | | | 14.6 | | |
| I-1167 | 34 | | 10 | | | 11.7 | | |
| I-1168 | 25 | | 10 | | | | | |
| I-1169 | 159 | | 10 | | | | | |
| I-1170 | 15 | | 10 | | | 13.5 | | |
| I-1171 | 70 | | −1 | | | | | |
| I-1172 | 45 | | −1 | | | | | |
| I-1173 | 12 | | 10 | | | 17.2 | | |
| I-1174 | 43 | | 10 | | | | | |
| I-1175 | 24 | | 10 | | | | | |
| I-1176 | 169 | | 10 | | | | | |
| I-1177 | 25 | | 35 | | | 4.3 | | |
| I-1178 | 54.5 | | −1 | | | | | |
| I-1179 | 346 | | 44 | | | | | |
| I-1180 | 87 | | 31 | | | | | |
| I-1181 | 225.5 | | 50 | | | | | |
| I-1182 | 144.5 | | 48 | | | | | |
| I-1183 | 409 | | 50 | | | | | |
| I-1184 | 175.5 | | 45 | | | | | |
| I-1185 | 203 | | 33 | | | | | |
| I-1186 | 127.5 | | 46 | | | | | |
| I-1187 | 80.5 | | 42 | | | | | |
| I-1188 | 22.5 | | 18 | | | 7.2 | | |
| I-1189 | 94.5 | | 10 | | | | | |
| I-1190 | 58.5 | | 10 | | | | | |
| I-1191 | 38.5 | | 10 | | | | | |
| I-1192 | 81.5 | | 10 | | | | | |
| I-1193 | 78.5 | | 10 | | | | | |
| I-1194 | 66.5 | | 10 | | | | | |
| I-1195 | 29.5 | | 10 | | | 16.2 | | |
| I-1196 | 85 | | 10 | | | | | |
| I-1197 | 44 | | 10 | | | | | |
| I-1198 | 65 | | 47 | | | | | |
| I-1199 | 44.5 | | 19 | | | | | |
| I-1200 | 99 | | 48 | | | | | |
| I-1201 | 13 | | 47 | | | | | |
| I-1202 | 16 | | 50 | | | | | |
| I-1203 | 16 | | 50 | | | 10.0 | | |
| I-1204 | 81 | | 50 | | | | | |
| I-1205 | 987 | | 50 | | | | | |
| I-1206 | 32 | | 50 | | | 27.8 | | |
| I-1207 | 40 | | 11 | | | 7.5 | | |
| I-1208 | 349 | | 44 | | | | | |
| I-1209 | 479 | | 16 | | | | | |
| I-1210 | 56 | | 50 | | | | | |
| I-1211 | 115 | | 11 | | | | | |
| I-1212 | 8663 | | −1 | | | | | |
| I-1213 | 10000 | | −1 | | | | | |
| I-1214 | 116 | | 50 | | | | | |
| I-1215 | 216 | | 10 | | | | | |
| I-1216 | 10000 | | 10 | | | | | |
| I-1217 | 10000 | | 15 | | | | | |
| I-1218 | 10 | | | | | >20 | | |
| I-1219 | 7 | | | | | >20 | | |
| I-1220 | 11 | | | | | 17.3 | | |
| I-1221 | 14 | | | | | 23.4 | | |
| I-1222 | 511 | | | | | | | |
| I-1223 | 550 | | | | | | | |
| I-1224 | 389 | | | | | | | |
| I-1225 | 305 | | | | | | | |
| I-1226 | 559 | | | | | | | |
| I-1227 | 10000 | | | | | | | |
| I-1228 | 100 | | | | | | | |
| I-1229 | 134 | | | | | | | |
| I-1230 | 10000 | | | | | | | |
| I-1231 | 10000 | | | | | | | |
| I-1232 | 31 | | | | | | | |
| I-1233 | 24 | | | | | | | |
| I-1234 | 41 | | | | | | | |
| I-1235 | 27 | | | | | | | |
| I-1236 | 69 | | | | | | | |
| I-1237 | 31 | | | | | | | |
| I-1238 | 44 | | | | | | | |
| I-1239 | 33 | | | | | | | |
| I-1240 | 33 | | | | | 11.5 | 4.9 | |
| I-1241 | 19 | | | | | 15.7 | 3.8 | |
| I-1242 | 30 | | | | | >>20 | 4.8 | |
| I-1243 | 82 | | | | | | | |
| I-1244 | 18 | | | | | 17.7 | 13.9 | |
| I-1245 | 9 | | | | | 12.2 | 7.3 | |
| I-1246 | 13 | | | | | 24.3 | 15.9 | |
| I-1247 | 33 | | | | | >20 | >20 | |
| I-1248 | 19 | | | | | 29.7 | | |
| I-1249 | 17 | | | | | 25.2 | | |
| I-1250 | 31 | | | | | >20 | | |
| I-1251 | 1940 | | | | | | | |
| I-1252 | 546 | | 44 | | | | | |
| I-1253 | 14 | | 42 | | | >20 | | |
| I-1254 | 530 | | −1 | | | | | |
| I-1255 | 32 | | 50 | | | | | |
| I-1256 | 1466 | | | | | | | |
| I-1257 | 11 | | | | | | | |
| I-1258 | 17 | | −1 | | | >20 | | |
| I-1259 | 10 | | −1 | | | 18.1 | | |
| I-1260 | 23 | | 50 | | | | | |
| I-1261 | 31 | | 50 | | | | | |
| I-1262 | 1124 | | 49 | | | | | |
| I-1263 | 43 | | 12 | | | 20.0 | | |
| I-1264 | 157 | | 10 | | | | | |
| I-1265 | 152 | | 10 | | | | | |
| I-1266 | 755 | | 10 | | | | | |
| I-1267 | 181 | | 10 | | | | | |
| I-1268 | 198 | | −1 | | | | | |
| I-1269 | 173 | | 10 | | | | | |
| I-1270 | 3699 | | 10 | | | | | |
| I-1271 | 682 | | 10 | | | | | |
| I-1272 | 331 | | 21 | | | | | |
| I-1273 | 9 | | 32 | | | >20 | | |
| I-1274 | 13 | | 10 | | | >20 | | |
| I-1275 | 9 | | −1 | | | 27.8 | | |
| I-1276 | 10 | | −1 | | | >20 | | |
| I-1277 | 17 | | −1 | | | >20 | | |
| I-1278 | 10 | | 25 | | | 18.0 | | |
| I-1279 | 12 | | 14 | | | 22.9 | | |
| I-1280 | 22 | | 10 | | | 24.7 | | |
| I-1281 | 10 | | 14 | | | | | |
| I-1282 | 34 | | 24 | | | | | |
| I-1283 | 26 | | 10 | | | | | |
| I-1284 | 10000 | | 10 | | | | | |
| I-1285 | 10000 | | 10 | | | | | |
| I-1286 | 10000 | | 10 | | | | | |
| I-1287 | 10000 | | 10 | | | | | |
| I-1288 | 1164 | | 10 | | | | | |
| I-1289 | 10000 | | 10 | | | | | |
| I-1290 | 10000 | | 10 | | | | | |
| I-1291 | 10000 | | 10 | | | | | |
| I-1292 | 10000 | | 10 | | | | | |
| I-1293 | 10000 | | −1 | | | | | |
| I-1294 | 10000 | | 10 | | | | | |
| I-1295 | 10000 | | 10 | | | | | |
| I-1296 | 3785 | | 10 | | | | | |
| I-1297 | 10000 | | −1 | | | | | |
| I-1298 | 10000 | | 10 | | | | | |
| I-1299 | 10000 | | 10 | | | | | |
| I-1300 | 10000 | | 10 | | | | | |
| I-1301 | 10000 | | −1 | | | | | |
| I-1302 | 45 | | −1 | | | | | |
| I-1303 | 86 | | 28 | | | | | |
| I-1304 | 31 | | 10 | | | | | |
| I-1305 | 6 | | 49 | | | 9.7 | | |
| I-1306 | 6 | | 50 | | | 6.6 | | |
| I-1307 | 21 | | 20 | | | | | |
| I-1308 | 25 | | 47 | | | | | |
| I-1309 | 59 | | 10 | | | | | |
| I-1310 | 124 | | 10 | | | | | |
| I-1311 | 38 | | 10 | | | | | |
| I-1312 | 14 | | 46 | | | | | |
| I-1313 | 12 | | 10 | | | | | |
| I-1314 | 9 | | 48 | | | >>20 | | |
| I-1315 | 13 | | 10 | | | 7.2 | | |
| I-1316 | 11 | | 48 | | | 6.8 | | |
| I-1317 | 9 | | 49 | | | 9.7 | | |
| I-1318 | 14 | | −1 | | | 4.8 | | |
| I-1319 | 10 | | 10 | | | 4.2 | | |
| I-1320 | 10 | | 44 | | | 26.2 | | |
| I-1321 | 8 | | 48 | | | 10.9 | | |
| I-1322 | 11 | | 33 | | | 9.1 | | |
| I-1323 | 6 | | 48 | | | 11.7 | | |
| I-1324 | 14 | | 25 | | | 5.8 | | |
| I-1325 | 14 | | 12 | | | 5.7 | | |
| I-1326 | 12 | | −1 | | | 6.2 | | |
| I-1327 | 13 | | 39 | | | 7.0 | | |
| I-1328 | 11 | | 26 | | | 5.4 | | |
| I-1329 | 23 | | 45 | | | 35.0 | | |
| I-1330 | 104 | | −1 | | | | | |
| I-1331 | 75 | | 10 | | | | | |
| I-1332 | 11 | | 13 | | | 2.5 | | |
| I-1333 | 20 | | 10 | | | 2.8 | | |
| I-1334 | 30 | | 10 | | | 4.2 | | |
| I-1335 | 13 | | 11 | | | 2.3 | | |
| I-1336 | 29 | | 10 | | | 2.7 | | |
| I-1337 | 85 | | 10 | | | 6.2 | | |
| I-1338 | 10 | | 10 | | | 3.6 | | |
| I-1339 | 33 | | 10 | | | 5.2 | | |
| I-1340 | 159 | | 10 | | | 6.8 | | |
| I-1341 | 17 | | −1 | | | 3.5 | | |
| I-1342 | 35 | | −1 | | | 7.0 | | |
| I-1343 | 150 | | 10 | | | | | |
| I-1344 | 11 | | 44 | | | 4.1 | | |
| I-1345 | 15 | | 37 | | | 4.7 | | |
| I-1346 | 2319 | | 50 | | | | | |
| I-1347 | 2369 | | −1 | | | | | |
| I-1348 | 1181 | | −1 | | | | | |
| I-1349 | 161 | | −1 | | | | | |
| I-1350 | 158 | | 28 | | | | | |
| I-1351 | 258 | | 14 | | | | | |
| I-1352 | 12 | | 50 | | | 19.5 | | |
| I-1353 | 18 | | 44 | | | 8.8 | | |
| I-1354 | 48 | | −1 | | | | | |
| I-1355 | 213 | | | | | | | |
| I-1356 | 23 | | 48 | | | >>20 | | |
| I-1357 | 10 | | 48 | | | 33.6 | | |
| I-1358 | 19 | | 10 | | | | | |
| I-1359 | 88 | | 34 | | | | | |
| I-1360 | 135 | | −1 | | | | | |
| I-1361 | 39 | | −1 | | | | | |
| I-1362 | 23 | | −1 | | | | | |
| I-1363 | 9 | | 12 | | | 5.5 | | |
| I-1364 | 9 | | 17 | | | | | |
| I-1365 | 9 | | 22 | | | | | |
| I-1366 | 7 | | 10 | | | | | |
| I-1367 | 31 | | 10 | | | | | |
| I-1368 | 44 | | 10 | | | | | |
| I-1369 | 29 | | 10 | | | | | |
| I-1370 | 18 | | 10 | | | | | |
| I-1371 | 22 | | 10 | | | | | |
| I-1372 | 29 | | 10 | | | | | |
| I-1373 | 5 | | 50 | | | | | |
| I-1374 | 17 | | 10 | | | 8.8 | | |
| I-1375 | 7 | | 50 | | | | | |
| I-1376 | 4208 | | 10 | | | | | |
| I-1377 | 1003 | | 10 | | | | | |
| I-1378 | 6171 | | 10 | | | | | |
| I-1379 | 1435 | | 10 | | | | | |
| I-1380 | 10000 | | 10 | | | | | |
| I-1381 | 10000 | | 10 | | | | | |
| I-1382 | 2853 | | 10 | | | | | |
| I-1383 | 1655 | | 10 | | | | | |
| I-1384 | 10000 | | 10 | | | | | |
| I-1385 | 1082 | | 10 | | | | | |
| I-1386 | 2248 | | 10 | | | | | |
| I-1387 | 6938 | | 10 | | | | | |
| I-1388 | 18 | | | | | | | |
| I-1389 | 9 | | | | | | | |
| I-1390 | 16 | | | | | | | |
| I-1391 | 11 | | | | | | | |
| I-1392 | 13 | | | | | | | |
| I-1393 | 34 | | | | | | | |
| I-1394 | 9 | | | | | | | |
| I-1395 | 33 | | | | | | | |
| I-1396 | 33 | | | | | | | |
| I-1397 | 48 | | | | | | | |
| I-1398 | 73 | | | | | | | |
| I-1399 | 13 | | | | | | | |
| I-1400 | 5 | | | | | | | |
| I-1401 | 5 | | | | | | | |
| I-1402 | 9 | | | | | | | |
| I-1403 | 6 | | | | | | | |
| I-1404 | 113 | | | | | | | |
| I-1405 | 9 | | | | | | | |
| I-1406 | 6 | | | | | | | |
| I-1407 | 5 | | | | | | | |
| I-1408 | 33 | | | | | | | |
| I-1409 | 74 | | | | | | | |
| I-1410 | 80 | | | | | | | |
| I-1411 | 109 | | | | | | | |
| I-1412 | 79 | | | | | | | |
| I-1413 | 74 | | | | | | | |
| I-1414 | 84 | | | | | | | |
| I-1415 | 72 | | | | | | | |
| I-1416 | 57 | | | | | | | |
| I-1417 | 198 | | | | | | | |
| I-1418 | 106 | | | | | | | |
| I-1419 | 152 | | | | | | | |
| I-1420 | 124 | | | | | | | |
| I-1421 | 134 | | | | | | | |
| I-1422 | 388 | | | | | | | |
| I-1423 | 389 | | | | | | | |
| I-1424 | 235 | | | | | | | |
| I-1425 | 239 | | | | | | | |
| I-1426 | 203 | | | | | | | |
| I-1427 | 33 | | | | | | | |
| I-1428 | 41 | | | | | | | |
| I-1429 | 10000 | | | | | | | |
| I-1430 | 10000 | | | | | | | |
| I-1431 | 34 | | | | | | | |
| I-1432 | 27 | | | | | | | |
| I-1433 | 71 | | | | | | | |
| I-1434 | 83 | | | | | | | |
| I-1435 | 90 | | | | | | | |
| I-1436 | 121 | | | | | | | |
| I-1437 | 7 | | | | | | | |
| I-1438 | 5 | | | | | | | |
| I-1439 | 349 | | | | | | | |
| I-1440 | 26 | | | | | | | |
| I-1441 | 327 | | | | | | | |
| I-1442 | 1500 | | | | | | | |
| I-1443 | 24 | | | | | | | |
| I-1444 | 6558 | | | | | | | |
| I-1445 | 13 | | | | | | | |
| I-1446 | 9 | | | | | | | |
| I-1447 | 11 | | | | | | | |
| I-1448 | 20 | | | | | | | |
| I-1449 | 16 | | | | | | | |
| I-1450 | 11 | | | | | | | |
| I-1451 | 13 | | | | | | | |
| I-1452 | 66 | | | | | | | |
| I-1453 | 52 | | | | | | | |
| I-1454 | 22 | | | | | | | |
| I-1455 | 13 | | | | | | | |
| I-1456 | 16 | | | | | | | |
| I-1457 | 43 | | | | | | | |

COLO320DM proliferation assay IC50: In some embodiments, inhibition of cell proliferation by provided technologies were assessed using cell lines related to or from certain conditions, disorders or diseases. In some embodiments, cell proliferation was assessed in COLO320DM cells. In some embodiments, assessment was performed using the following procedure: On Day 1, cultured COLO320DM cells in a T75 flask were trypsinized in 3 mL of 0.25% trypsin/EDTA for 5 min and quenched with 10 mL RPMI-1640+4% HI FBS assay media. The cells were spun down at 1200 rpm for 5 min, the cell pellet collected and re-suspended at 5000 cells/mL in assay media. Using a Combi liquid handler, cells were dispensed (50 uL, 250 cells/well) into three 384 well plates. Plates were incubated at 37° C., 500 CO2 for 18-22 h. On day 2, compounds were added. A liquid handling system was used to prepare the compound dilution and dispense compound into assay plates. The compounds were serially diluted 1/2 in 90% DMSO to create a 7 point dose curve. From compound plate, 100 nL of compound were dispensed directly into wells of the assay plates to create a dose curve starting at 20 uM and ending at 313 nM. Assay plates were incubated at 37° C., 500 CO2 for 96 h. On day 6, assay plates were removed from the incubator and allowed to sit at room temperature for 30 min. Using a liquid handler, 20 uL of CellTiter Glo reagent was added to each well. The assay plates were shaken for 2 min and allowed to sit on the bench for 10-15 minutes. The assay plates were read using the CellTiter Glo protocol on a GloMax microplate reader, and the data analyzed using GraphPad Prism. Activities of various agents, including various stapled peptides in Table E2, were confirmed. Certain results are presented in Table E2-1 below.

Table E2-1. Certain data of various peptide compositions.
Structural information are described in Table E3. Compositions of stapled peptides.
1. Compound ID
2. beta-Catenin FP IC50 (nM)
3. NanoBRET Abs IC50 (uM)
4. DLD1 4% Abs IC50 (uM)
5. COLO320DM Proliferation Abs IC50 (uM)
6. Calculated Mass
7. Found m/z (positive mode)
8. Found m/z (negative mode)
9. C═C double bond (e.g., —CH═CH—) conversion. A: —CH═CH— in each staple reduced to —CH₂—CH₂—; B: —CH═CH— in N-terminal side staple converted to an epoxide moiety

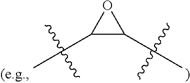 [SMILES: *C1OC1*]

| 1 | 2 | 3 | 4 | 5 | 6 | 7 | 8 | 9 |
|---|---|---|---|---|---|---|---|---|
| I-1 | | | | | 1822.857 | | | |
| I-2 | | | | | 1822.857 | | | |
| I-3 | | | | | 1834.857 | 918.8 | 1834.2 | |
| I-4 | | | | | 1834.857 | 918.8 | 1834.2 | |
| I-5 | | | | | 1848.872 | 925.8 | 1848.2 | |
| I-6 | | | | | 1848.872 | 925.8 | 1848.2 | |
| I-7 | | | | | 1896.872 | 949.8 | 1896.2 | |
| I-8 | | | | | 1896.872 | 949.8 | 1896.2 | |
| I-9 | | | | | 1853.878 | 1855.5 | 1853.2 | |
| I-10 | | | | | 1853.878 | 1855.6 | 1853.2 | |
| I-11 | | | | | 1928.914 | 1952.5 | 1928.2 | |
| I-12 | | | | | 1928.914 | 1952.6 | 1928.2 | |
| I-13 | | | | | 1818.898 | 1842.4 | 1818.2 | |
| I-14 | | | | | 1818.898 | 1842.5 | 1818.2 | |
| I-15 | | | | | 1858.929 | 1882.5 | 1858.2 | |
| I-16 | | | | | 1858.929 | 1882.5 | 1858.2 | |
| I-17 | | | | | 1832.914 | 1856.5 | 1832.2 | |
| I-18 | | | | | 1832.914 | 1856.5 | 1832.2 | |
| I-19 | | | | | 1864.831 | 1888.4 | 1864.1 | |
| I-20 | | | | | 1864.831 | 1888.4 | 1864.1 | |
| I-21 | | | | | 1878.846 | 1902.4 | 1878.2 | |
| I-22 | | | | | 1878.846 | 1902.4 | 1878.2 | |
| I-23 | | | | | 1892.862 | 1916.4 | 1892.2 | |
| I-24 | | | | | 1892.862 | 1916.4 | 1892.2 | |
| I-25 | | | | | 1808.841 | 1810.4 | 1808.1 | |
| I-26 | | | | | 1822.857 | 1824.4 | 1822.1 | |
| I-27 | | | | | 1808.841 | 1810.4 | 1808.1 | |
| I-28 | | | | | 1822.857 | 1824.4 | 1822.1 | |
| I-29 | | | | | 1822.857 | 1829.3 | 1805 | |
| I-30 | | | | | 1806.825 | 1808.3 | 1806.1 | |
| I-31 | | | | | 1806.825 | 1830.3 | 1806.1 | |
| I-32 | | | | | 1792.81 | 1794.3 | 1792 | |
| I-33 | | | | | 1792.81 | 1794.3 | 1792 | |
| I-34 | | | | | 1822.857 | 1824.3 | 1822.1 | |
| I-35 | | | | | 1806.825 | 1830.2 | 1806 | |
| I-36 | | | | | 1794.825 | 1818.3 | 1794 | |
| I-37 | | | | | 1794.825 | 1796.3 | 1794 | |
| I-38 | | | | | 1792.81 | 1816.3 | 1792 | |
| I-39 | | | | | 1806.825 | 1808.3 | 1806.1 | |
| I-40 | | | | | 1806.825 | 1808.3 | 1806.1 | |
| I-41 | | | | | 1780.81 | 1782.3 | 1780 | |
| I-42 | | | | | 1780.81 | 1782.3 | 1780 | |
| I-43 | | | | | 1822.857 | 1824.3 | 1822 | |
| I-44 | | | | | 1822.857 | 1824.4 | 1822 | |
| I-45 | | | | | 1808.841 | 1810.3 | 1808 | |
| I-46 | | | | | 1808.841 | 1810.3 | 1808 | |
| I-47 | | | | | 1735.825 | 1737.3 | 1735 | |
| I-48 | | | | | 1735.825 | 1737.3 | 1735 | |
| I-49 | | | | | 1794.825 | 1796.3 | 1794 | |
| I-50 | | | | | 1794.825 | 1796.3 | 1794 | |
| I-51 | | | | | 1792.81 | 1794.3 | 1792 | |
| I-52 | 3195 | | | | 1779.887 | 1781.5 | | |
| I-53 | 5000 | | | | 1824.872 | 1826.5 | | |
| I-54 | 3187 | | | | 1824.872 | 1826.6 | | |
| I-55 | 5000 | | | | 1810.857 | 1812.5 | | |
| I-56 | | | | | 1866.898 | 1868.6 | | |
| I-57 | | | | | 1866.898 | 1868.6 | | |
| I-58 | | | | | 1833.873 | 1835.6 | | |
| I-59 | | | | | 1833.873 | 1835.7 | | |
| I-60 | | | | | 1826.888 | 1850.6 | 1826.3 | |
| I-61 | | | | | 1826.888 | 1850.6 | 1826.3 | |
| I-62 | | | | | 1836.836 | 1838.6 | 1936.2 | |
| I-63 | | | | | 1836.836 | 1838.6 | 1936.2 | |
| I-64 | 4246 | | | | 1826.863 | 1828.7 | | |
| I-65 | | | | | 1826.863 | 1828.7 | 1826.4 | |
| I-66 | 1090 | | | | 1840.878 | 1842.8 | 1840.5 | |
| I-67 | 1227 | | | | 1840.878 | 1842.7 | | |
| I-68 | | | | | 1824.92 | 1826.8 | | |
| I-69 | | | | | 1824.92 | 1826.7 | | |
| I-70 | | | | | 1838.936 | 1840.8 | | |
| I-71 | | | | | 1838.936 | 1840.9 | 1838.5 | |
| I-72 | | | | | 1864.951 | 1867 | 1864.6 | |
| I-73 | | | | | 1864.951 | 1866.8 | | |
| I-74 | | | | | 1826.888 | 1850.6 | 1826.3 | |
| I-75 | | | | | 1826.888 | 1850.6 | 1826.3 | |
| I-76 | | | | | 1786.857 | 1788.6 | 1786.2 | |
| I-77 | | | | | 1838.832 | 1840.7 | 1838.4 | |
| I-78 | 5000 | | | | 1838.832 | 1840.6 | | |
| I-79 | | | | | 1856.822 | 1858.6 | | |
| I-80 | | | | | 1821.836 | 1823.7 | 1821.4 | |
| I-81 | | | | | 1821.836 | 1823.7 | 1821.4 | |
| I-82 | | | | | 1821.836 | 1823.7 | 1821.4 | |
| I-83 | | | | | 1821.836 | 1823.7 | 1821.4 | |
| I-84 | | | | | 1821.836 | 1823.8 | 1821.4 | |
| I-85 | | | | | 1821.836 | 1823.7 | 1821.3 | |
| I-86 | | | | | 1821.836 | 1823.7 | 1821.3 | |
| I-87 | | | | | 1821.836 | 1823.7 | 1821.4 | |
| I-88 | | | | | 1821.836 | 1823.7 | 1821.4 | |
| I-89 | | | | | 1863.847 | 1187.8 | 1163.5 | |
| I-90 | | | | | 1863.847 | 1865.8 | | |
| I-91 | | | | | 1863.847 | 1865.9 | | |
| I-92 | | | | | 1863.847 | 1887.7 | 1863.3 | |
| I-93 | | | | | 1812.872 | 1814.7 | | |
| I-94 | | | | | 1812.872 | 1814.8 | | |
| I-95 | | | | | 1812.872 | 1814.9 | | |
| I-96 | | | | | 1812.872 | 1814.7 | | |
| I-97 | | | | | 1866.898 | 1868.7 | | |
| I-98 | | | | | 1866.898 | 1868.8 | | |
| I-99 | | | | | 1852.882 | 1854.8 | | |
| I-100 | | | | | 1852.882 | 1876.8 | 1852.4 | |
| I-101 | | | | | 1850.852 | 1852.6 | | |
| I-102 | | | | | 1850.852 | 1852.6 | | |
| I-103 | | | | | 1850.852 | 1852.9 | | |
| I-104 | | | | | 1850.852 | 1852.7 | | |
| I-105 | | | | | 1863.847 | 1887.5 | | |
| I-106 | | | | | 1863.847 | 1865.6 | | |
| I-107 | | | | | 1863.847 | | | |
| I-108 | | | | | 1863.847 | 1865.6 | | |
| I-109 | | | | | 1863.847 | 1887.6 | | |
| I-110 | | | | | 1864.831 | 1866.6 | | |
| I-111 | | | | | 1864.831 | 1866.7 | | |
| I-112 | | | | | 1870.857 | 1872.6 | | |
| I-113 | | | | | 1870.857 | 1872.6 | | |
| I-114 | | | | | 1902.898 | 1904.7 | | |
| I-115 | | | | | 1902.898 | 1904.8 | | |
| I-116 | | | | | 1902.898 | 1904.8 | | |
| I-117 | | | | | 1834.857 | 1836.6 | | |
| I-118 | | | | | 1834.857 | 1858.9 | 1834.2 | |
| I-119 | | | | | 1850.852 | 1852.7 | 1850.4 | |
| I-120 | | | | | 1850.852 | 1852.8 | 1850.4 | |
| I-121 | | | | | 1788.8 | 1790.4 | | |
| I-122 | | | | | 1788.8 | 1790.6 | | |
| I-123 | 2112 | | | | 1793.903 | 1795.8 | 1793.4 | |
| I-124 | | | | | 1778.903 | 1780.8 | 1778.4 | |
| I-125 | 3059 | | | | 1778.903 | 890.9. | 1778.4 | |
| I-126 | | | | | 1792.919 | 1794.8 | | |
| I-127 | 4841 | | | | 1792.919 | 1794.8 | | |
| I-128 | | | | | 1665.844 | 1667.7 | | |
| I-129 | 1981 | | | | 1807.919 | 905.6 | 1808.5 | |
| I-130 | | | | | 1838.888 | 1840.8 | | |
| I-131 | 2160 | | | | 1838.888 | 1840.8 | | |
| I-132 | 5000 | | | | 1779.887 | 1781.8 | | |
| I-133 | 5000 | | | | 1750.897 | | 1751.4 | |
| I-134 | 2696 | | | | 1750.897 | | 1751.2 | |
| I-135 | 1867 | | | | 1736.881 | 1760.7 | 1736.4 | |
| I-136 | | | | | 1807.919 | 1809.9 | | |
| I-137 | | | | | 1807.919 | 1810.1 | 1807.6 | |
| I-138 | | | | | 1777.944 | 1628.9 | | |
| I-139 | | | | | 1777.944 | 1779.9 | | |
| I-140 | 2269 | | | | 1804.908 | 1806.8 | 1804.5 | |
| I-141 | | | | | 1676.849 | 1700.8 | 1676.4 | |
| I-142 | | | | | 1822.898 | 1824.9 | 1822.6 | |
| I-143 | 2272 | | | | 1805.903 | 1807.8 | 1805.5 | |
| I-144 | 146.5 | | | | 1810.864 | 1812.8 | 1810.5 | |
| I-145 | | | | | 1830.923 | 1832.9 | 1830.5 | |
| I-146 | | | | | 1811.893 | 1813.8 | 1811.4 | |
| I-147 | 1637 | | | | 1717.835 | 1719.8 | 1717.4 | |
| I-148 | 2772 | | | | 1703.82 | 1705.7 | 1703.3 | |
| I-149 | | | | | 1755.826 | 1757.8 | | |
| I-150 | | | | | 1755.826 | 1757.7 | | |
| I-151 | | | | | 1618.767 | 1642.6 | | |
| I-152 | 5000 | | | | 1783.857 | 1785.8 | | |
| I-153 | 2135 | | | | 1774.857 | 1776.8 | | |
| I-154 | 5000 | | | | 1717.835 | 1719.7 | | |
| I-155 | 5000 | | | | 1717.835 | 1719.7 | | |
| I-156 | | | | | 1798.831 | 1729.7 | | |
| I-157 | | | | | 1772.841 | 1796.7 | 1772.4 | |
| I-158 | | | | | 1772.841 | 1774.8 | 1772.5 | |
| I-159 | | | | | 1729.835 | 1753.7 | 1729.4 | |
| I-160 | | | | | 1729.835 | | 1729.4 | |
| I-161 | | | | | 1802.815 | 1804.7 | 1802.4 | |
| I-162 | | | | | 1802.815 | 1804.8 | 1802.5 | |
| I-163 | | | | | 1808.805 | 1810.7 | | |
| I-164 | | | | | 1808.805 | 1832.7 | 1808.5 | |
| I-165 | | | | | 1823.913 | 1825.8 | 1823.6 | |
| I-166 | 2164 | | | | 1811.893 | 1813.8 | 1811.5 | |
| I-167 | | | | | 1795.919 | 1797.9 | | |
| I-168 | | | | | 1795.919 | 1797.9 | | |
| I-169 | | | | | 1861.89 | 1863.9 | | |
| I-170 | | | | | 1833.934 | 1836.1 | | |
| I-171 | | | | | 1833.934 | 1838 | | |
| I-172 | 5000 | | | | 1836.945 | 1838.9 | | |
| I-173 | 3116 | | | | 1836.945 | 1839 | | |
| I-174 | 1760 | | | | 1859.925 | 1862.3 | 1860.3 | |
| I-176 | 1992 | | | | 1844.95 | 1847.3 | 1845.4 | |
| I-178 | 1633 | | | | 1831.893 | 1834.2 | 1832.1 | |
| I-180 | 56 | | | | 1864.94 | 934.1 | 1865.4 | |
| I-181 | 310 | | | | 1864.94 | 1867.4 | 1865.6 | |
| I-182 | 2800 | | | | 1793.903 | 1796.2 | | |
| I-183 | | | | | 1883.856 | 1886.3 | 1884.2 | |
| I-184 | 3109 | | | | 1840.889 | 1843.2 | 1841.4 | |
| I-185 | | | | | 1755.836 | 879.5 | 1756.2 | |
| I-186 | 5000 | | | | 1708.85 | 856 | 1709.1 | |
| I-187 | 3318 | | | | 1708.85 | 856 | | |
| I-188 | 1597 | | | | 1904.91 | 1907.4 | 1905.4 | |
| I-190 | 696 | | | | 1904.91 | 1907.4 | | |
| I-191 | 1656 | | | | 1736.881 | 870 | | |
| I-193 | 533 | | | | 1736.881 | 870 | | |
| I-194 | 1008 | | | | 1807.919 | 905.6 | | |
| I-195 | | | | | 1825.944 | 1828.4 | 1826.3 | |
| I-196 | | | | | 1839.96 | 1842.6 | | |
| I-197 | | | | | 1839.96 | 1842.3 | 1840.6 | |
| I-198 | 5000 | | | | 1792.919 | 898.1 | 1793.4 | |
| I-199 | 5000 | | | | 1792.919 | 898.1 | 1793.4 | |
| I-200 | 5000 | | | | 1792.919 | 898.1 | 1793.3 | |
| I-201 | | | | | 1825.944 | 1828.4 | 1826.2 | |
| I-202 | | | | | 1841.939 | 1844.4 | 1842.5 | |
| I-203 | | | | | 1839.96 | 1842.4 | | |
| I-204 | | | | | 1843.935 | 1946.4 | | |
| I-205 | | | | | 1791.96 | 897.6 | 1792.4 | |
| I-206 | | | | | 1815.935 | 1818.3 | 1816.5 | |
| I-207 | 1745 | | | | 1807.919 | 1810.3 | | |
| I-209 | | | | | 1847.875 | 1850.3 | | |
| I-211 | 5000 | | | | 1847.875 | 1850.3 | 1848.3 | |
| I-213 | | | | | 1826.94 | 1829.4 | 1827.4 | |
| I-214 | | | | | 1826.94 | 1829.5 | 1827.5 | |
| I-215 | | | | | 1791.96 | 1794.3 | 1792.5 | |
| I-217 | 1957 | | | | 1823.913 | 1826.2 | 1824.4 | |
| I-219 | 2855 | | | | 1829.884 | 1832.5 | 1830.3 | |
| I-221 | | | | | 1822.82 | 913 | 1823.3 | |
| I-223 | | | | | 1822.82 | 913 | 1823.4 | |
| I-225 | | | | | 1910.794 | 957 | 1911.2 | |
| I-227 | | | | | 1910.794 | 1913 | 1911 | |
| I-229 | | | | | 1826.94 | 1929.3 | 1927.5 | |
| I-230 | | | | | 1826.94 | 1929.3 | 1927.2 | |
| I-231 | | | | | 1867.901 | 1870.7 | 1868.4 | |
| I-232 | | | | | 1861.944 | 1964.4 | 1862.3 | |
| I-233 | | | | | 1850.94 | 1853.4 | 1851.4 | |
| I-234 | | | | | 1861.944 | 1864.4 | 1862.6 | |
| I-235 | | | | | 1887.96 | 945.7 | 1888.4 | |
| I-236 | 5000 | | | | 1847.875 | 1850.2 | 1848.4 | |
| I-238 | 1592 | | | | 1847.875 | 1850.2 | 1848.4 | |
| I-240 | | | | | 1847.875 | 1850.2 | 1848.3 | |
| I-242 | | | | | 1847.875 | 925.6 | | |
| I-244 | | | | | 1879.916 | 1882 | 1880 | |
| I-246 | | | | | 1879.916 | 1882.3 | | |
| I-248 | | | | | 1847.875 | 1850.3 | | |
| I-250 | 5000 | | | | 1847.875 | 1850.2 | | |
| I-252 | | | | | 1847.875 | 1850.3 | | |
| I-254 | 5000 | | | | 1847.875 | 1850.3 | 1848.3 | |
| I-256 | | | | | 1879.935 | 1882 | 1880 | |
| I-257 | | | | | 1829.919 | 1832 | 1830 | |
| I-258 | | | | | 1829.919 | 1832 | 1830 | |
| I-259 | | | | | 1847.91 | 1850 | 1848 | |
| I-260 | 5000 | | | | 1797.878 | 1800 | 1798 | |
| I-261 | 5000 | | | | 1797.878 | 1800 | 1798 | |
| I-262 | 5000 | | | | 1815.868 | 1818 | 1816 | |
| I-263 | 5000 | | | | 1815.868 | 1818 | 1816 | |
| I-264 | 5000 | | | | 1797.878 | 1800 | 1798 | |
| I-265 | 3762 | | | | 1797.878 | 1800 | 1798 | |
| I-266 | 5000 | | | | 1797.878 | 1800 | 1798 | |
| I-267 | 5000 | | | | 1815.868 | 1818 | 1816 | |
| I-268 | | | | | 1793.903 | 1796 | 1794 | |
| I-269 | | | | | 1809.898 | 1812 | 1810 | |
| I-270 | 5000 | | | | 1812.924 | 1815 | 1813 | |
| I-271 | 5000 | | | | 1812.924 | 1815 | 1813 | |
| I-272 | | | | | 1812.924 | 1815 | 1813 | |
| I-273 | | | | | 1841.939 | 1844 | 1842 | |
| I-274 | | | | | 1827.924 | 1830 | 1828 | |
| I-275 | 5000 | | | | 1829.903 | 1832 | 1830 | |
| I-276 | 3428 | | | | 1829.903 | 1832 | 1830 | |
| I-277 | | | | | 1829.903 | 1832 | 1830 | |
| I-278 | | | | | 1829.903 | 1832 | 1830 | |
| I-279 | | | | | 1793.903 | 1796 | 1794 | |
| I-280 | | | | | 1793.903 | 1796 | 1794 | |
| I-281 | | | | | 1793.903 | 1796 | 1794 | |
| I-282 | | | | | 1793.903 | 1796 | 1794 | |
| I-283 | | | | | 1747.846 | 1750 | 1748 | |
| I-284 | | | | | 1746.862 | 1749 | 1747 | |
| I-285 | 5000 | | | | 1769.878 | 1772 | 1770 | |
| I-286 | 4482 | | | | 1694.834 | 849 | 1694.9 | |
| I-287 | 4954 | | | | 1751.856 | 1754 | 1752 | |
| I-288 | 1313 | | | | 1751.856 | 1754 | 1752 | |
| I-289 | 5000 | | | | 1651.829 | 827.5 | | |
| I-290 | 3286.5 | | | | 1651.829 | 827.5 | | |
| I-291 | 3129 | | | | 1722.866 | 863 | | |
| I-292 | 1871 | | | | 1722.866 | 863 | | |
| I-293 | 139.5 | | | | 1907.878 | 955.6 | 1908.1 | |
| I-294 | 130 | | | | 1825.839 | 914.6 | 1826.3 | |
| I-295 | 5000 | | | | 1907.896 | 955.7 | 1908 | |
| I-296 | | | | | 1850.874 | 827.1 | 1851.2 | |
| I-297 | | | | | 1878.906 | 941.2 | 1879.2 | |
| I-298 | | | | | 1866.869 | 935.1 | 1867.5 | |
| I-299 | 1303 | | | | 1798.828 | 901.1 | 1799.3 | |
| I-300 | | | | | 1879.901 | 941.7 | 1880.5 | |
| I-301 | | | | | 1842.906 | 923.1 | 1843.4 | |
| I-302 | | | | | 1886.801 | 1889.1 | 1887.2 | |
| I-303 | | | | | 1887.96 | 1890.3 | 1888.3 | |
| I-304 | 5000 | | | | 1801.919 | 1804.1 | 1802.2 | |
| I-305 | 3982.5 | | | | 1801.919 | 1804.1 | 1802.2 | |
| I-306 | 5000 | | | | 1813.848 | 908.8 | 1814 | |
| I-307 | 4196 | | | | 1813.848 | 908.8 | 1814 | |
| I-308 | 5000 | | | | 1813.848 | 908.8 | | |
| I-309 | 3118 | | | | 1813.848 | 908.8 | | |
| I-310 | | | | | 1813.848 | 908.8 | | |
| I-311 | | | | | 1813.848 | 908.8 | | |
| I-312 | | | | | 1813.848 | 908.8 | | |
| I-313 | | | | | 1813.848 | 908.8 | | |
| I-314 | | | | | 1815.935 | 909.7 | | |
| I-315 | | | | | 1815.935 | 909.6 | | |
| I-316 | 3982 | | | | 1751.856 | 877.5 | | |
| I-317 | 5000 | | | | 1797.878 | 900.6 | | |
| I-318 | 4633 | | | | 1797.878 | 900.6 | | |
| I-319 | 1419 | | | | 1797.878 | 900.6 | | |
| I-320 | | | | | 1769.878 | 886.6 | | |
| I-321 | | | | | 1769.878 | 886.6 | | |
| I-322 | 5000 | | | | 1794.898 | 899.1 | | |
| I-323 | 3471 | | | | 1794.898 | 899.1 | | |
| I-324 | | | | | 1826.94 | 915.2 | | |
| I-325 | 1310 | | | | 1785.844 | 894.6 | | |
| I-326 | 1752 | | | | 1785.844 | 894.6 | 1786.4 | |
| I-327 | 4724 | | | | 1769.866 | 886.5 | 1770.3 | |
| I-328 | 1914 | | | | 1769.866 | 886.5 | 1770.3 | |
| I-329 | 5000 | | | | 1783.893 | 1786.3 | | |
| I-330 | 5000 | | | | 1783.893 | 1786.1 | | |
| I-331 | 5000 | | | | 1783.893 | 1786 | | |
| I-332 | 5000 | | | | 1783.893 | 1786.1 | | |
| I-333 | 2756 | | | | 1726.824 | 871 | 1739.1 | |
| I-334 | 5000 | | | | 1738.861 | 865 | 1727.2 | |
| I-335 | 1544 | | | | 1738.861 | 865 | 1727 | |
| I-336 | 153 | | | | 1839.854 | 1842.1 | | |
| I-337 | 5000 | | | | 1839.854 | 921.6 | 1840.2 | |
| I-338 | 5000 | | | | 1839.854 | 921.6 | 1840.2 | |
| I-339 | 5000 | | | | 1840.838 | 922.1 | 1841.2 | |
| I-340 | 5000 | | | | 1840.838 | 1843.2 | 1841.3 | |
| I-341 | 2000 | | | | 1812.843 | 1815 | 1813.3 | |
| I-342 | 58.5 | | | | 1798.828 | 1801.1 | 1799.3 | |
| I-343 | 172 | | | | 1811.823 | 1814.1 | 1812.3 | |
| I-344 | 42 | | | | 1811.823 | 1814.1 | 1812 | |
| I-345 | 134 | | | | 1826.859 | 1829.2 | 1827.2 | |
| I-346 | 180 | | | | 1853.87 | 1856.2 | 1854.3 | |
| I-347 | 121.5 | | | | 1853.87 | 1856.2 | 1854.3 | |
| I-348 | 5000 | | | | 1735.897 | 869.5 | | |
| I-349 | 5000 | | | | 1735.897 | 869.5 | 1736 | |
| I-350 | 5000 | | | | 1735.897 | 869.6 | 1736.3 | |
| I-351 | 5000 | | | | 1735.897 | 869.5 | | |
| I-352 | 5000 | | | | 1735.897 | 869.5 | | |
| I-353 | 5000 | | | | 1703.856 | 853.5 | 1704.1 | |
| I-354 | 5000 | | | | 1703.856 | 853.5 | 1704.3 | |
| I-355 | 5000 | | | | 1703.856 | 1706 | 1704.2 | |
| I-356 | 5000 | | | | 1703.856 | 853.5 | 1704.1 | |
| I-357 | 5000 | | | | 1664.845 | 834 | 1665.2 | |
| I-358 | 5000 | | | | 1664.845 | 834 | | |
| I-359 | 5000 | | | | 1821.844 | 1824.2 | 1822.3 | |
| I-360 | 145.5 | | | | 1834.839 | 1837.1 | | |
| I-361 | 42 | | | | 1834.839 | 1837.1 | | |
| I-362 | 3656 | | | | 1758.808 | 1761.1 | | |
| I-363 | 5000 | | | | 1758.808 | 1761 | | |
| I-364 | 5000 | | | | 1758.808 | 1761.1 | | |
| I-365 | 2715 | | | | 1782.833 | 893.1 | 1783.4 | |
| I-366 | 1445 | | | | 1782.833 | 1785 | 1783.1 | |
| I-367 | 177 | | | | 1782.833 | 1785.1 | 1783.3 | |
| I-368 | 5000 | | | | 1808.883 | 906.1 | 1809.3 | |
| I-369 | 2311 | | | | 1782.833 | 893.6 | 1784.2 | |
| I-370 | 1563 | | | | 1818.833 | 1821.2 | 1819.2 | |
| I-371 | 1389 | | | | 1786.808 | 895.1 | | |
| I-372 | 2093 | | | | 1800.823 | 1803.4 | | |
| I-373 | 945 | | | | 1864.836 | 941.1 | 1879.2 | |
| I-374 | 3011 | | | | 1836.804 | 1853.4 | 18951 | |
| I-376 | 1321 | | | | 1836.804 | 927.1 | 1851.4 | |
| I-378 | 1336 | | | | 1893.826 | 955.6 | 1908.3 | |
| I-380 | 552 | | | | 1893.826 | 955.6 | 1908.2 | |
| I-381 | 3653 | | | | 1813.855 | 1816.3 | 1814.3 | |
| I-382 | 2064 | | | | 1813.855 | 908.6 | 1814.3 | |
| I-383 | 5000 | | | | 1811.859 | 1814.3 | 1812.2 | |
| I-384 | 177 | | | | 1811.859 | 1814.2 | 1812 | |
| I-385 | 3150 | | | | 1811.859 | 907.6 | | |
| I-386 | 5000 | | | | 1811.859 | 1814.3 | | |
| I-387 | 2621 | | | | 1811.859 | 1814.3 | | |
| I-388 | 5000 | | | | 1846.845 | 1877 | 1875.1 | |
| I-389 | 5000 | | | | 1846.845 | 939 | 1875.1 | |
| I-390 | 3422 | | | | 1846.845 | 1877 | 1875 | |
| I-391 | 1913 | | | | 1846.845 | 1876.8 | 1875 | |
| I-392 | 5000 | | | | 1802.895 | 895.5 | | |
| I-393 | 1953 | | | | 1802.895 | 895.5 | | |
| I-394 | 5000 | | | | 1846.845 | 1214.5 | 1212.4 | |
| I-395 | 5000 | | | | 1824.88 | 1192.5 | 1190.4 | |
| I-396 | 3427 | | | | 1836.871 | | 1937.3 | |
| I-397 | 2229 | | | | 1836.871 | 1839.1 | 1837.2 | |
| I-398 | 2155 | | | | 1881.856 | 1884.3 | | |
| I-399 | 2499 | | | | 1865.861 | 1868.2 | 1866 | |
| I-400 | 3587 | | | | 1910.846 | 1913.1 | 1911.2 | |
| I-401 | 134 | | | | 1863.801 | | 1863.8 | |
| I-402 | 5000 | | | | 1852.837 | 1855.1 | 1853.2 | |
| I-403 | 2128 | | | | 1850.887 | 1853 | 1851.2 | |
| I-404 | 1851 | | | | 1968.874 | | 1969.3 | |
| I-405 | 3821 | | | | 2013.859 | | 2014.4 | |
| I-406 | 94 | | | | 1839.854 | 1841.9 | | |
| I-407 | 30 | | | | 1881.901 | 1884 | | |
| I-408 | 120 | | | | 1753.843 | 878.5 | 1754.2 | |
| I-409 | 70 | | | | 1753.843 | 878.5 | 1754 | |
| I-410 | 50 | | | | 1867.885 | 1869.9 | | |
| I-411 | 189 | | | | 1824.88 | 1826.9 | | |
| I-412 | 92 | | | | 1905.848 | | 1906.1 | |
| I-413 | 90 | | | | 1849.861 | 926.5 | | |
| I-414 | 3417 | | | | 1863.877 | 1866 | | |
| I-415 | 2201 | | | | 1863.877 | 1866 | | |
| I-416 | 5000 | | | | 1863.877 | 1866 | | |
| I-417 | 5000 | | | | 1863.877 | 1866 | | |
| I-418 | 149 | | | | 1848.855 | 1851 | | |
| I-419 | 23 | | | | 1848.855 | 1850.9 | | |
| I-420 | 127.5 | | | | 1821.844 | 1824.1 | 1822.1 | |
| I-421 | 99 | | | | 1834.839 | 1836.9 | | |
| I-422 | 27 | | | | 1834.839 | 1836.9 | | |
| I-423 | 105 | | | | 1812.843 | | 1813.2 | |
| I-424 | 170 | | | | 1825.839 | 1828.4 | | |
| I-425 | 50 | | | | 1825.839 | | 1826.3 | |
| I-426 | 109 | | | | 1845.811 | 1848.4 | 1846.6 | |
| I-427 | 38.5 | | | | 1845.811 | 1848.2 | 1848.4 | |
| I-428 | 60 | | | | 1845.811 | 1848.2 | 1846.2 | |
| I-429 | 146 | | | | 1863.801 | 1866.2 | 1864.3 | |
| I-430 | 154 | | | | 1872.802 | 1875.1 | 1873 | |
| I-431 | 1320 | | | | 1735.743 | | 1736.1 | |
| I-432 | 145 | | | | 1697.78 | | 1697.8 | |
| I-433 | 136 | | | | 1812.843 | | 1813.1 | |
| I-434 | 67.5 | | | | 1843.829 | 1845.9 | 1844 | |
| I-435 | 70 | | | | 1859.8 | 1862.4 | | |
| I-436 | 56 | | | | 1903.749 | 1906.9 | 1905.1 | |
| I-437 | 26 | 17.5 | | | 1855.849 | 1857.9 | 1856.1 | |
| I-438 | 5000 | | | | 1829.845 | 1831.9 | 1830.3 | |
| I-439 | 5000 | | | | 1829.845 | 1831.9 | 1830 | |
| I-440 | 5000 | | | | 1829.845 | 1831.9 | 1830.1 | |
| I-441 | 133 | | | | 1870.824 | 1872.8 | 1870.8 | |
| I-442 | 4484 | | | | 1839.854 | 1841.9 | 1840.1 | |
| I-443 | 2126 | | | | 1839.854 | 1841.9 | 1840 | |
| I-444 | 80 | | | | 1835.845 | | 1836 | |
| I-445 | 4962 | | | | 1835.845 | | 1835.9 | |
| I-446 | 130 | | | | 1831.795 | 1833.9 | 1832 | |
| I-447 | 74 | | | | 1831.795 | | 1832 | |
| I-448 | 92 | | | | 1831.795 | 1833.9 | 1831.9 | |
| I-449 | 53 | | | | 1831.795 | | 1831.8 | |
| I-450 | 3717 | | | | 1846.84 | 1848.9 | 1846.9 | |
| I-451 | 5000 | | | | 1846.84 | | 1847 | |
| I-452 | 1656 | | | | 1846.84 | | 1846.8 | |
| I-453 | 1554 | | | | 1843.829 | 1845.8 | 1844 | |
| I-454 | 125 | | | | 1843.829 | 923.5. | 1844.2 | |
| I-455 | 83.5 | | | | 1843.829 | 923.5. | 1844.2 | |
| I-456 | 2168 | | | | 1839.854 | 921.5. | 1840.1 | |
| I-457 | 1435 | | | | 1839.854 | 921.5. | 1839.9 | |
| I-458 | 101.5 | | | | 1839.854 | 921.5. | 1840.1 | |
| I-459 | 183 | | | | 1839.854 | 921.5. | 1840.1 | |
| I-460 | 195.5 | | | | 1815.818 | 909.5. | 1815.9 | |
| I-461 | 125 | | | | 1840.849 | 1842.9 | 1841.2 | |
| I-462 | 28 | >>20 | | | 1868.808 | 1870.4 | | |
| I-463 | 92 | | | | 1882.824 | 1884.4 | | |
| I-464 | 57 | | | | 1882.824 | 1884.4 | | |
| I-465 | 81 | | | | 1820.823 | 1822.8 | 1820.8 | |
| I-466 | 19 | | | | 1820.823 | 1822.8 | 1820.9 | |
| I-467 | 44 | | | | 1853.87 | 1855.8 | | |
| I-468 | 27 | | | | 1853.87 | 1855.8 | | |
| I-469 | 27 | | | | 1862.87 | 1864.8 | | |
| I-470 | 11 | | | | 1862.87 | 1864.8 | | |
| I-471 | 52 | | | | 1890.901 | 1892.8 | | |
| I-472 | 62 | | | | 1876.886 | 1878.8 | | |
| I-473 | 121 | | | | 1839.854 | 1841.7 | | |
| I-474 | 43 | | | | 1839.854 | 1841.7 | | |
| I-475 | 102 | | | | 1867.885 | 1869.8 | | |
| I-476 | 48 | | | | 1867.885 | 1869.8 | | |
| I-477 | 119 | | | | 1869.865 | 1871.7 | | |
| I-478 | 71 | | | | 1848.855 | 1850.9 | | |
| I-479 | 27 | | | | 1848.855 | 1850.8 | | |
| I-480 | 176 | | | | 1848.855 | 1850.9 | | |
| I-481 | 42 | | | | 1848.855 | 1850.9 | | |
| I-482 | 118 | | | | 1839.891 | 1841.8 | | |
| I-483 | 5000 | | | | 1860.876 | 932 | | |
| I-484 | 5000 | | | | 1860.876 | 932 | | |
| I-485 | 34 | 11.3 | | | 1887.858 | 1889.8 | | |
| I-486 | 147 | | | | 1845.811 | 1847.8 | | |
| I-487 | 45 | | | | 1845.811 | 1847.7 | | |
| I-488 | 43 | | | | 1896.858 | 1898.8 | | |
| I-489 | 57 | | | | 1882.842 | 1884.8 | | |
| I-490 | 88 | | | | 1848.843 | | 1848.7 | |
| I-491 | 153 | | | | 1812.843 | | 1812.7 | |
| I-492 | 53 | | | | 1812.843 | | 1812.8 | |
| I-493 | 88 | | | | 1798.828 | | 1798.8 | |
| I-494 | 82 | | | | 1839.854 | 1841.8 | | |
| I-495 | 181 | | | | 1867.885 | 1869.8 | 1867.9 | |
| I-496 | 5000 | | | | 1825.875 | 1827.8 | | |
| I-497 | 5000 | | | | 1825.875 | | 1825.8 | |
| I-498 | 5000 | | | | 1825.875 | 1827.8 | 1825.8 | |
| I-499 | 123 | | | | 1945.868 | 1947.8 | | |
| I-500 | 176 | | | | 1987.915 | 1989.9 | | |
| I-501 | 94 | | | | 1907.842 | 1909.8 | | |
| I-502 | 55 | | | | 1907.842 | 1909.7 | | |
| I-503 | 84 | | | | 1889.851 | 1891.8 | | |
| I-504 | 65 | | | | 1916.842 | 1918.8 | | |
| I-505 | 23 | 4.9 | 9.4 | | 1881.901 | 1883.9 | | |
| I-506 | 25 | 8.9 | | | 1890.901 | 1892.9 | | |
| I-507 | 192 | | | | 1812.843 | 907.9 | | |
| I-508 | 73 | | | | 1812.843 | 907.9 | | |
| I-509 | 99 | | | | 1796.848 | 899.9 | | |
| I-510 | 128 | | | | 1864.849 | 1866.9 | | |
| I-511 | 40 | | | | 1864.849 | 1866.8 | | |
| I-512 | 20 | 12.3 | | | 1906.896 | 1908.9 | | |
| I-513 | 143 | | | | 1877.845 | 1879.7 | | |
| I-514 | 30 | >>20 | | | 1919.892 | 1921.8 | | |
| I-515 | 54 | | | | 1840.795 | 1842.9 | | |
| I-516 | 23 | 30.5 | | | 1840.795 | 1842.8 | | |
| I-517 | 15 | 9.7 | | | 1882.842 | 1884.9 | | |
| I-518 | 143 | | | | 1831.795 | 917.4 | | |
| I-519 | 33 | 28.8 | | | 1831.795 | 917.4 | | |
| I-520 | 45 | | | | 1873.842 | 938.4 | | |
| I-521 | 23 | 15.4 | | | 1873.842 | 938.4 | | |
| I-522 | 75 | | | | 1867.885 | 935.4 | | |
| I-523 | 44 | | | | 1867.885 | 935.4 | | |
| I-524 | 2550 | | | | 1832.79 | 1834.6 | | |
| I-525 | 196 | | | | 1869.865 | 1871.6 | 1869.6 | |
| I-526 | 56 | | | | 1869.865 | 1871.6 | | |
| I-527 | 98 | | | | 1911.912 | 1913.7 | | |
| I-528 | 63 | | | | 1911.912 | 1913.7 | 1911.7 | |
| I-529 | 5000 | | | | 1874.837 | 939 | | |
| I-530 | 5000 | | | | 1874.837 | 939 | | |
| I-531 | 5000 | | | | 1888.853 | 946 | | |
| I-532 | 5000 | | | | 1888.853 | 946 | | |
| I-533 | 155 | | | | 1860.822 | 931.9 | | |
| I-534 | 5000 | | | | 1874.837 | 938.9 | | |
| I-535 | 5000 | | | | 1837.911 | 1839.8 | | |
| I-536 | 4469 | | | | 1823.896 | 1825.7 | | |
| I-537 | 5000 | | | | 1837.911 | 1839.8 | | |
| I-538 | 5000 | | | | 1835.896 | 1837.7 | | |
| I-539 | 5000 | | | | 1849.911 | 1851.8 | | |
| I-540 | 5000 | | | | 1809.88 | 906.4 | | |
| I-541 | 180 | | | | 1883.88 | 943.4 | | |
| I-542 | 109 | | | | 1883.88 | 943.4 | | |
| I-543 | 32 | 17.0 | | | 1876.886 | 1878.8 | 1876.6 | |
| I-544 | 24 | 4.6 | | | 1895.917 | 1897.6 | 1895.5 | |
| I-545 | 21 | 3.5 | | | 1895.917 | 1897.6 | 1895.5 | |
| I-546 | 16 | 12.1 | | | 1867.885 | 1869.5 | | |
| I-547 | 43 | | | | 1867.885 | 1869.4 | | |
| I-548 | 17 | 15.6 | | | 1867.885 | 1869.4 | | |
| I-549 | 71 | | | | 1883.88 | 1885.4 | | |
| I-550 | 30 | 21.4 | | | 1883.88 | 1885.4 | | |
| I-551 | 45 | | | | 1859.826 | 1861.4 | | |
| I-552 | 15.5 | | | | 1859.826 | 1861.4 | | |
| I-553 | 61 | | | | 1892.881 | 1894.7 | | |
| I-554 | 27.5 | | | | 1892.881 | 1894.6 | | |
| I-555 | 41 | | | | 1868.827 | 1870.7 | | |
| I-556 | 20 | | | | 1868.827 | 1870.6 | | |
| I-557 | 35 | 16.9 | | | 1867.885 | 1869.5 | | |
| I-558 | 56 | | | | 1867.885 | 1869.5 | | |
| I-559 | 43 | | | | 1867.885 | 1869.5 | | |
| I-560 | 81 | | | | 1876.886 | 1878.7 | | |
| I-561 | 34 | | | | 1876.886 | 1878.7 | | |
| I-562 | 51 | | | | 1853.87 | 1855.5 | | |
| I-563 | 19 | 15.7 | | | 1853.87 | 1855.5 | | |
| I-564 | 36 | >>20 | | | 1862.87 | 1864.7 | | |
| I-565 | 13 | 26.0 | | | 1862.87 | 1864.7 | | |
| I-566 | 76 | | | | 1839.854 | 1841.5 | | |
| I-567 | 37 | | | | 1839.854 | 1841.5 | | |
| I-568 | 73 | | | | 1853.87 | 1855.5 | | |
| I-569 | 42 | | | | 1853.87 | 1855.5 | | |
| I-570 | 56 | | | | 1862.87 | 1864.7 | | |
| I-571 | 32 | 15.6 | | | 1862.87 | 1864.7 | | |
| I-572 | 73 | | | | 1825.839 | 1827.4 | | |
| I-573 | 36 | 33.3 | | | 1825.839 | 1827.4 | | |
| I-574 | 36 | >>20 | | | 1834.839 | 1836.7 | | |
| I-575 | 21 | | | | 1834.839 | 1836.7 | | |
| I-576 | 23 | 11.1 | | | 1867.885 | 1869.5 | | |
| I-577 | 13 | 10.4 | | | 1867.885 | 1869.5 | | |
| I-578 | 5000 | | | | 1881.901 | 1883.7 | 1881.7 | |
| I-579 | 5000 | | | | 1877.862 | 1879.6 | 1877.6 | |
| I-580 | 114 | | | | 1876.886 | 1878.8 | 1876.7 | |
| I-581 | 112 | | | | 1876.886 | 1878.8 | 1876.7 | |
| I-582 | 17.5 | 15.8 | | | 1876.886 | 1878.8 | 1876.7 | |
| I-583 | 69 | | | | 1876.886 | 1878.8 | 1876.7 | |
| I-584 | 33 | | | | 1876.886 | 1878.8 | 1876.8 | |
| I-585 | 5000 | | | | 1881.901 | 1883.7 | 1881.6 | |
| I-586 | 5000 | | | | 1881.901 | 1883.7 | 1881.6 | |
| I-598 | 845 | | | | 1944.912 | 973.9. | 1944.6 | |
| I-599 | 138 | | | | 1944.912 | 973.9. | 1944.7 | |
| I-600 | 796 | | | | 1957.932 | 1959.7 | 1957.7 | |
| I-601 | 419 | | | | 1957.932 | 1959.7 | 1957.7 | |
| I-602 | 3150 | | | | 1972.943 | 988.98 | 1972.6 | |
| I-603 | 392 | | | | 1880.917 | 941.9. | 1880.7 | |
| I-604 | 692 | | | | 1909.944 | 1911.7 | 1909.6 | |
| I-605 | 632 | | | | 1909.944 | 1911.7 | 1909.7 | |
| I-606 | 289 | | | | 1943.917 | 1945.7 | 1943.7 | |
| I-607 | 253 | | | | 1943.917 | 1945.7 | 1943.7 | |
| I-608 | 211 | | | | 1924.943 | 1926.7 | 1924.7 | |
| I-609 | 80 | | | | 1924.943 | 1926.7 | 1924.7 | |
| I-610 | 54 | | | | 1978.915 | 1980.7 | 1978.6 | |
| I-611 | 55 | | | | 1978.915 | 1980.7 | 1978.6 | |
| I-612 | 64 | | | | 1895.917 | 1897.7 | 1895.7 | |
| I-613 | 1627 | | | | 1908.96 | 1910.8 | 1908.6 | |
| I-614 | 1143 | | | | 1908.96 | 1910.8 | 1908.8 | |
| I-615 | 50 | | | | 1916.917 | 1918.8 | 1916.7 | |
| I-616 | 108 | | | | 1919.928 | 1921.8 | 1919.6 | |
| I-617 | 253 | | | | 1863.891 | 1865.9 | 1863.7 | |
| I-618 | 135 | | | | 1863.891 | 1865.7 | 1863.7 | |
| I-619 | 44 | | | | 1907.917 | 1909.6 | 1907.6 | |
| I-620 | 35 | 12.0 | | | 1907.917 | 1909.5 | 1907.5 | |
| I-621 | 58 | | | | 1910.928 | 1912.5 | 1910.5 | |
| I-622 | 230 | | | | 1854.89 | 1856.5 | 1854.5 | |
| I-623 | 103 | | | | 1854.89 | 1856.4 | 1854.5 | |
| I-624 | 273 | | | | 1739.827 | 871.3 | 1739.4 | |
| I-625 | 119 | | | | 1739.827 | 871.3 | 1739.4 | |
| I-626 | 5000 | | | | 1779.858 | 891.3 | 1779.4 | |
| I-627 | 338 | | | | 1779.858 | 891.3 | 1779.4 | |
| I-628 | 60 | | | | 1782.869 | 892.8 | 1782.6 | |
| I-629 | 36 | 30.0 | | | 1782.869 | 892.8 | 1782.3 | |
| I-630 | 5000 | | | | 1984.871 | | | |
| I-631 | 5000 | | | | 1984.871 | | | |
| I-632 | 5000 | | | | 1984.871 | | | |
| I-633 | 88 | | | | 1909.944 | 1911.7 | 1909.5 | |
| I-634 | 425 | | | | 1866.927 | 934.9 | 1866.5 | |
| I-635 | 171 | | | | 1900.911 | 951.9 | 1900.5 | |
| I-636 | 198 | | | | 1916.906 | 959.9 | 1916.5 | |
| I-637 | 5000 | | | | 1850.895 | 926.9 | 1850.4 | |
| I-638 | 3876 | | | | 1850.895 | 926.8 | 1850.5 | |
| I-639 | 109 | | | | 1840.875 | 921.8 | 1840.4 | |
| I-640 | 87 | | | | 1840.875 | 921.8 | 1840.4 | |
| I-641 | 122 | | | | 1854.89 | 928.8 | 1854.4 | |
| I-642 | 118 | | | | 1854.89 | 928.8 | 1854.4 | |
| I-643 | 958 | | | | 1939.922 | 971.4 | 1939.5 | |
| I-644 | 808 | | | | 1939.922 | 971.4 | 1939.5 | |
| I-645 | 395 | | | | 1852.911 | 927.9 | 1852.4 | |
| I-646 | 461 | | | | 1852.911 | 927.8 | 1852.4 | |
| I-647 | 445 | | | | 1923.948 | 1925.5 | 1923.5 | |
| I-648 | 233 | | | | 1923.948 | 1925.5 | 1923.4 | |
| I-649 | 209 | | | | 1957.932 | 1959.5 | 1957.5 | |
| I-650 | 137 | | | | 1957.932 | 1959.4 | 1957.4 | |
| I-651 | 186 | | | | 1897.896 | 1899.4 | 1897.3 | |
| I-652 | 72 | | | | 1897.896 | 1899.4 | 1897.3 | |
| I-653 | 108 | | | | 1938.923 | 1940.4 | 1938.4 | |
| I-654 | 20 | 15.0 | | | 1938.923 | 1940.4 | 1938.4 | |
| I-655 | 183 | | | | 1996.943 | 1998.5 | 1996.5 | |
| I-656 | 73 | | | | 1923.948 | 1925.5 | 1923.4 | |
| I-657 | 46 | | | | 1923.948 | 1925.5 | 1923.4 | |
| I-658 | 546 | | | | 1923.948 | 963.3 | 1923.4 | |
| I-659 | 430 | | | | 1923.948 | 963.4 | 1923.4 | |
| I-660 | 544 | | | | 1957.932 | 980.4 | | |
| I-661 | 45 | | | | 1897.896 | 950.5 | | |
| I-662 | 26 | 16.1 | | | 1897.896 | 950.5 | | |
| I-663 | 43 | | | | 1938.923 | 971 | | |
| I-664 | 996 | | | | 1996.943 | 999.8 | | |
| I-665 | 923 | | | | 1996.943 | 999.7 | | |
| I-666 | 626 | | | | 1923.948 | | | |
| I-667 | 585 | | | | 1923.948 | | | |
| I-668 | 675 | | | | 1881.901 | | | |
| I-669 | 451 | | | | 1881.901 | | | |
| I-670 | 5000 | | | | 1895.917 | | | |
| I-671 | 3138 | | | | 1895.917 | | | |
| I-672 | 5000 | | | | 1895.917 | | | |
| I-673 | 5000 | | | | 1881.901 | | | |
| I-674 | 5000 | | | | 1881.901 | | | |
| I-675 | 4525 | | | | 1895.917 | | | |
| I-676 | 5000 | | | | 1895.917 | | | |
| I-677 | 22 | 6.7 | | | 1881.901 | 1883.3 | 1881.3 | |
| I-678 | 26 | 16.8 | | | 1873.842 | 1875.3 | 1873.3 | |
| I-679 | 7 | 17.1 | | | 1873.842 | 1875.3 | 1873.3 | |
| I-680 | 32 | 34.4 | | | 1859.826 | 1861.3 | 1859.3 | |
| I-681 | 12 | 25.1 | | | 1859.826 | 1861.3 | 1859.3 | |
| I-682 | 41 | | | | 1887.858 | 1889.3 | 1887.3 | |
| I-683 | 19 | 6.7 | | | 1887.858 | 1889.3 | 1887.3 | |
| I-684 | 15 | 14.3 | | | 1924.871 | 1926.3 | 1924.3 | |
| I-685 | 25 | 19.4 | | | 1924.871 | 1926.3 | 1924.3 | |
| I-686 | 5 | 12. | | | 1916.811 | 1918.3 | 1916.3 | |
| I-687 | 12 | 29.3 | | | 1916.811 | 1918.3 | 1916.3 | |
| I-688 | 6 | 30.1 | | | 1902.796 | 1904.3 | 1902.3 | |
| I-689 | 11 | 33.9 | | | 1902.796 | 1904.3 | 1902.3 | |
| I-690 | 7 | 17.8 | | | 1930.827 | 1932.3 | 1930.3 | |
| I-691 | 9 | 17.0 | | | 1930.827 | 1932.4 | 1930.4 | |
| I-692 | 9 | 25.6 | | | 1933.871 | 1935.7 | 1933.3 | |
| I-693 | 12 | 28.5 | | | 1933.871 | 1935.7 | 1933.4 | |
| I-694 | 5 | | | | 1925.812 | 1927.7 | 1925.4 | |
| I-695 | 5 | | | | 1925.812 | 1927.7 | 1925.4 | |
| I-696 | 5 | | | | 1911.796 | 1913.6 | 1911.3 | |
| I-697 | 5 | 14.7 | | | 1939.827 | 1941.7 | 1939.4 | |
| I-698 | 5 | | | | 1939.827 | 1941.7 | 1939.5 | |
| I-699 | 43 | | | | 1964.899 | 1966.4 | 1964.4 | |
| I-700 | 49 | | | | 1970.856 | 1972.4 | 1970.4 | |
| I-701 | 34 | 18.1 | | | 1970.856 | 1972.4 | 1970.4 | |
| I-702 | 76 | | | | 1979.856 | 1981.6 | 1979.5 | |
| I-703 | 57 | 11.9 | | | 1978.915 | 1980.5 | 1978.5 | |
| I-704 | 49 | | | | 1984.871 | 1986.5 | 1984.5 | |
| I-705 | 58 | | | | 1993.872 | 1995.8 | 1993.5 | |
| I-706 | 57 | | | | 2004.931 | 2006.5 | 2004.5 | |
| I-707 | 46 | | | | 2010.887 | 2012.5 | 2010.5 | |
| I-708 | 52 | | | | 2010.887 | 2012.5 | 2010.5 | |
| I-709 | 54 | | | | 2019.887 | 2021.8 | 2019.5 | |
| I-710 | 73 | | | | 1917.868 | 1919.2 | 1917.2 | |
| I-711 | 88 | | | | 1917.868 | 1919.3 | 1917.2 | |
| I-712 | 92 | | | | 1753.843 | 878.1 | 1753.1 | |
| I-713 | 70 | | | | 1753.843 | 878.1 | 1753.1 | |
| I-714 | 131 | | | | 1739.827 | 871.1 | 1739.1 | |
| I-715 | 60.5 | | | | 1739.827 | 871.1 | 1739 | |
| I-716 | 91 | | | | 1745.783 | 874.1 | 1745.1 | |
| I-717 | 39 | | | | 1745.783 | 874.1 | 1745.1 | |
| I-718 | 82 | | | | 1759.799 | 881.1 | 1759.2 | |
| I-719 | 38 | | | | 1759.799 | 881.1 | 1759.1 | |
| I-720 | 75 | | | | 1745.783 | 874.1 | 1745.1 | |
| I-721 | 46 | | | | 1745.783 | 874.1 | 1745.1 | |
| I-722 | 1128 | | | | 1887.948 | 945.3 | 1887.2 | |
| I-723 | 3833 | | | | 1957.932 | 980.3 | 1957.3 | |
| I-724 | 114 | | | | 1911.912 | 957.2 | 1911.2 | |
| I-725 | 5000 | | | | 1957.932 | 1959.2 | 1957.2 | |
| I-726 | 5000 | | | | 1867.885 | 1869.2 | 1867.2 | |
| I-727 | 5000 | | | | 1861.932 | 1863.2 | 1861.2 | |
| I-728 | 289 | | | | 1847.917 | 1849.2 | 1847.2 | |
| I-729 | 56 | | | | 1938.923 | 970.8 | 1938.3 | |
| I-730 | 35 | 11.2 | | | 1938.923 | 970.8 | 1938.2 | |
| I-731 | 34 | 11.6 | | | 1938.923 | 970.8 | 1938.3 | |
| I-732 | 4219 | | | | 1980.97 | 1982.3 | 1980.3 | |
| I-733 | 4261 | | | | 1980.97 | 991.8 | 1980.3 | |
| I-734 | 370 | | | | 1980.97 | 991.7 | 1980.4 | |
| I-735 | 246 | | | | 1980.97 | 991.7 | 1980.3 | |
| I-736 | 440 | | | | 1952.938 | 991.7 | 1980.3 | |
| I-737 | 353 | | | | 1952.938 | 991.7 | 1980.3 | |
| I-738 | 63 | | | | 1947.923 | 1949.5 | 1947.3 | |
| I-739 | 53 | | | | 1947.923 | 1949.6 | 1947.3 | |
| I-740 | 376 | | | | 1989.97 | 1991.7 | 1989.3 | |
| I-741 | 226 | | | | 1989.97 | 1991.6 | 1989.4 | |
| I-742 | 159 | | | | 1989.97 | 1991.7 | 1989.4 | |
| I-743 | 85 | | | | 1961.939 | 1963.7 | 1961.3 | |
| I-744 | 112 | | | | 1876.886 | 1878.5 | 1876.2 | |
| I-745 | 80 | | | | 1876.886 | 1878.5 | 1876.2 | |
| I-746 | 5000 | | | | 1890.901 | 1892.6 | 1890.3 | |
| I-747 | 2388 | | | | 1890.901 | 1892.6 | 1890.3 | |
| I-748 | 54 | | | | 1840.875 | 921.7 | 1840.3 | |
| I-749 | 40 | 9.0 | | | 1840.875 | 921.7 | 1840.2 | |
| I-750 | 43 | 7.4 | | | 1826.859 | 914.7 | 1826.2 | |
| I-751 | 29 | 7.1 | | | 1826.859 | 914.6 | 1826.1 | |
| I-752 | 16 | 9.9 | | | 1853.87 | 928.1 | 1853.2 | |
| I-753 | 14 | 10.0 | | | 1853.87 | 928.1 | 1853.2 | |
| I-754 | 115 | | | | 1840.875 | 921.6 | 1840.1 | |
| I-755 | 88 | | | | 1840.875 | 921.6 | 1840.1 | |
| I-756 | 56 | | | | 1826.859 | 914.6 | 1826.1 | |
| I-757 | 64 | | | | 1853.87 | 928.1 | 1853.1 | |
| I-758 | 47 | | | | 1853.87 | 928.1 | 1853.1 | |
| I-759 | 1534 | | | | 1840.875 | 1842.2 | 1840.1 | |
| I-760 | 608 | | | | 1840.875 | 1842.3 | 1840 | |
| I-761 | 5 | | | | 2027.841 | 2029.1 | 2027 | |
| I-762 | 5 | 14.0 | 7.9 | | 2027.841 | 2029 | 2027 | |
| I-763 | 5 | 21.3 | | | 1930.827 | 1932.1 | 1930 | |
| I-764 | 14 | 30.9 | | | 1930.827 | 1932 | 1930 | |
| I-765 | 228 | | | | 1918.827 | 1920.1 | 1918 | |
| I-766 | 55 | | | | 1918.827 | 1920.1 | 1918 | |
| I-767 | 12 | 37.2 | | | 1930.827 | 1932.1 | 1930 | |
| I-768 | 9 | 31.4 | | | 1930.827 | 1932.1 | 1930.1 | |
| I-769 | 5 | 13.0 | | | 2036.841 | 2038.4 | 2036.1 | |
| I-770 | 5 | 13.7 | | | 2036.841 | 2038.4 | 2036.1 | |
| I-771 | 5 | 37.3 | | | 1939.827 | 1941.5 | 1939.1 | |
| I-772 | 7 | >>20 | | | 1939.827 | 1941.4 | 1939.1 | |
| I-773 | 167 | | | | 1927.827 | 1929.4 | 1927 | |
| I-774 | 38 | | | | 1927.827 | 1929.5 | 1927.1 | |
| I-775 | 6 | >>20 | | | 1939.827 | 1941.4 | 1939.1 | |
| I-776 | 5 | 45.7 | | | 1939.827 | 1941.4 | 1939.1 | |
| I-777 | 5 | 19.8 | | | 1919.855 | 1921.6 | 1919.4 | |
| I-778 | 8 | 11.2 | | | 1916.811 | 1918.3 | 1916.3 | |
| I-779 | 5 | 18.7 | | | 1925.812 | 1927.6 | 1925.2 | |
| I-780 | 9 | 20.7 | | | 1940.865 | 1942.3 | 1940.2 | |
| I-781 | 5 | 31.0 | | | 1949.866 | 1951.5 | 1949.2 | |
| I-782 | 6 | 12.2 | | | 1933.871 | 1935.6 | 1933.2 | |
| I-783 | 7 | 6.2 | | | 1944.816 | 973.9 | 971.8 | |
| I-784 | 6 | 14.5 | | | 1953.816 | 1955.9 | 1953.7 | |
| I-785 | 6 | 21.6 | | | 1788.753 | 895.5 | 1787.9 | |
| I-786 | 8 | 29.3 | | | 1802.768 | 902.5 | 1801.9 | |
| I-787 | 5 | 30.7 | | | 1916.811 | 1918 | 1915.9 | |
| I-788 | 5 | 21.6 | | | 1925.812 | 1927.4 | 1924.9 | |
| I-789 | 19 | >>20 | | | 1919.855 | 1921.2 | 1918.8 | |
| I-790 | 24 | >>20 | | | 1919.855 | 1921.2 | 1918.8 | |
| I-791 | 310 | | | | 1942.812 | 1944.8 | 1943.3 | |
| I-792 | 20 | >>20 | | | 1908.851 | 1910.4 | 1908 | |
| I-793 | 14 | >>20 | | | 1975.827 | 1977.4 | 1975.1 | |
| I-794 | 19 | 26.4 | | | 1969.871 | 1971.4 | 1969 | |
| I-795 | 176 | | | | 1969.871 | 1971.4 | 1968.8 | |
| I-798 | 106 | | | | 1942.812 | 1844.8 | 1942.3 | |
| I-799 | 92 | | | | 1922.866 | 1924.3 | 1921.9 | |
| I-800 | 79 | | | | 1926.841 | 1928.3 | 1925.9 | |
| I-801 | 7 | 5.8 | 4.3 | >20 | 1915.852 | 1917.1 | 1915 | |
| I-802 | 6 | 4.8 | | | 1924.853 | 1926.4 | 1924 | |
| I-803 | 5 | 9.6 | 9.0 | | 1903.816 | 1905 | 1903 | |
| I-804 | 5 | 13.5 | | | 1912.816 | 1914.4 | 1911.9 | |
| I-805 | 5 | 18.5 | | | 1959.854 | 1961.2 | 1959 | |
| I-806 | 5 | 21.6 | | | 1968.854 | 1970.4 | 1968 | |
| I-807 | 5 | 3.2 | | | 1949.837 | 1951.1 | 1949 | |
| I-808 | 5 | 4.4 | | | 1958.837 | 1960.4 | 1958 | |
| I-809 | 5 | 6.1 | 11.3 | | 1903.816 | 1905 | 1902.9 | |
| I-810 | 6 | 18.0 | | | 1912.816 | 1914.4 | 1911.9 | |
| I-811 | 32 | >>20 | | | 1917.807 | 1919.2 | | |
| I-812 | 33 | 48.6 | | | 1923.763 | 1925.2 | | |
| I-813 | 7 | 20.9 | | | 1925.812 | 1927.4 | | |
| I-814 | 7 | 18.9 | 16.5 | | 1889.8 | 946.1 | | |
| I-815 | 5 | 15.4 | 10.3 | | 1875.785 | 939.1 | | |
| I-816 | 5 | 13.7 | | | 1902.796 | 1904 | | |
| I-817 | 10 | | | | 1889.8 | 946 | | |
| I-818 | 6 | 15.0 | | | 1902.796 | 1904 | | |
| I-819 | 5 | 7.5 | | | 1987.848 | 1989 | 1987 | |
| I-820 | 6 | 18.2 | | | 1996.849 | 1998.4 | 1996 | |
| I-821 | 5 | 9.1 | | | 1973.833 | 1975.1 | 1973 | |
| I-822 | 6 | 6.3 | 7.2 | | 1987.848 | 1989 | 1987 | |
| I-823 | 5 | 18.3 | | | 1996.849 | 1998.4 | 1996 | |
| I-824 | 7 | 14.7 | | | 1973.833 | 1975.1 | 1973 | |
| I-825 | 15 | 27.5 | | | 2001.864 | 2003 | 2001 | |
| I-826 | 15 | 22.7 | | | 2001.864 | 1002.1 | 2001 | |
| I-827 | 13 | >>20 | | | 2010.864 | 2012.4 | 2010 | |
| I-828 | 13 | | | | 2010.864 | 1006.7 | 2010 | |
| I-829 | 15 | 30.2 | | | 1987.848 | 1989.1 | 1987 | |
| I-830 | 15 | 38.5 | | | 2001.864 | 1002.2 | 2001 | |
| I-831 | 11 | 25.6 | | | 2001.864 | 1002.2 | 2001 | |
| I-832 | 9 | 39.8 | | | 2010.864 | 2012.3 | 2010 | |
| I-833 | 8 | 48.1 | | | 2010.864 | 2012.3 | 2010 | |
| I-834 | 14 | 33.7 | | | 1987.848 | 1989.1 | 1987 | |
| I-835 | 11 | 37.6 | | | 1987.848 | 1989 | 1987 | |
| I-836 | 11 | 16.9 | | | 1915.827 | 1917.1 | 1915 | |
| I-837 | 5 | 23.8 | | | 1924.828 | 1926.4 | 1924.1 | |
| I-838 | 12 | 17.4 | | | 1929.843 | 1931.1 | 1929.1 | |
| I-839 | 7 | 41.9 | | | 1938.843 | 1940.4 | 1938.9 | |
| I-840 | 16 | 22.3 | | | 1958.87 | 1960.2 | 1958.1 | |
| I-841 | 12 | 34.4 | | | 1967.87 | 1969.4 | 1967.1 | |
| I-842 | 34 | 47.9 | | | 1964.848 | 1966.1 | 1964 | |
| I-843 | 154 | | | | 1973.848 | 1975.5 | 1973.1 | |
| I-844 | 6 | | | | 1902.832 | 1904.1 | 1902.1 | |
| I-845 | 7 | | | | 1911.832 | 1913.5 | 1911 | |
| I-846 | 56 | | | | 1914.868 | 1916.1 | 1914.1 | |
| I-847 | 28 | >>20 | | | 1923.869 | 1925.5 | 1923.1 | |
| I-848 | 13 | 7.0 | | | 2060.866 | 2061.9 | 2059.9 | |
| I-849 | 8 | 3.6 | 1.1 | 9.2 | 2026.882 | 1014.6 | 2026 | |
| I-850 | 26 | 16.4 | | | 2025.868 | 1014.1 | 2025.1 | |
| I-851 | 16 | 7.6 | | | 1991.884 | 997.1 | 1991 | |
| I-852 | 7 | 2.8 | 1.4 | | 2025.868 | 1014.1 | 2025.1 | |
| I-853 | 8 | 6.6 | | | 2025.868 | 1014.1 | 2025.1 | |
| I-854 | 8 | 3.6 | | | 1991.884 | 997.1 | 1991 | |
| I-855 | 22 | | | | 1948.853 | 1949.9 | 1947.9 | |
| I-856 | 17 | 6.8 | | | 1914.868 | 1916 | 1914 | |
| I-857 | 15 | 30.6 | | | 1787.769 | 1788.9 | 1787 | |
| I-858 | 34 | >>20 | | | 1801.784 | | 1800.9 | |
| I-859 | 36 | >>20 | | | 1774.774 | | 1773.9 | |
| I-860 | 61 | | | | 1774.774 | | 1773.9 | |
| I-861 | 40 | | | | 1830.811 | | 1829.9 | |
| I-862 | 123 | | | | 1786.81 | | 1785.9 | |
| I-863 | 22 | >>20 | | | 1810.785 | 1940 | 1937.9 | |
| I-864 | 12 | 40.0 | | | 1938.843 | 1812 | 1809.8 | |
| I-870 | 9.5 | 14.6 | | | 2129.923 | 2131 | 2128.8 | |
| I-871 | 5 | 13.7 | | | 2129.923 | 1917.7 | 1915.7 | |
| I-872 | 9.5 | 14.1 | | | 2129.923 | 2130.7 | 2128.8 | |
| I-873 | 6.5 | 10.0 | | | 2129.923 | 2131 | 2128.8 | |
| I-874 | 18.5 | 14.6 | | | 2086.953 | 1044.6 | 2085.8 | |
| I-875 | 26 | 13.7 | | | 2086.953 | 2087.8 | 2085.8 | |
| I-876 | 24 | 13.2 | | | 2086.953 | 2087.7 | 2085.8 | |
| I-877 | 11 | 15.6 | | | 2086.953 | 2088 | 2085.8 | |
| I-881 | 10 | 23.8 | | | 2015.916 | 1009.2 | 1007 | |
| I-882 | 22 | 41.4 | | | 2015.916 | 1009.1 | 1007.1 | |
| I-883 | 53 | | | | 2241.901 | 1122.1 | 1120.1 | |
| I-884 | 14 | >>20 | | | 2155.942 | 1079.1 | 1077.1 | |
| I-885 | 32 | >>20 | | | 2181.958 | 1092.1 | 1090.1 | |
| I-886 | 22 | >>20 | | | 2100.933 | 1051.6 | 1049.5 | |
| I-887 | 14 | >>20 | | | 2113.964 | 1058.1 | 1056.1 | |
| I-888 | 13 | | | | 2182.027 | 1092.1 | 1090.1 | |
| I-889 | 16 | >>20 | | | 2189.995 | 1096.2 | 1094.1 | |
| I-890 | 16 | >>20 | | | 2176.964 | 1089.6 | 1087.6 | |
| I-891 | 13 | | | | 2134.953 | 1068.6 | 1066.6 | |
| I-892 | 18 | >>20 | | | 2103.932 | 1053 | 1051 | |
| I-893 | 834 | | | | 2103.932 | 1053.6 | 1051.6 | |
| I-894 | 15 | >>20 | | | 2191.985 | 1097.1 | 1095 | |
| I-895 | 14 | >>20 | | | 2280.037 | 1141.1 | 1139.1 | |
| I-896 | 22 | >>20 | | | 2368.089 | 1185.2 | 1183.1 | |
| I-897 | 16 | >>20 | | | 2043.911 | 1023.1 | 1021 | |
| I-898 | 16 | >>20 | | | 2099.974 | 1051.1 | 1049.1 | |
| I-899 | 28 | >>20 | | | 2135.974 | 1069.1 | 1067.1 | |
| I-900 | 33 | >>20 | | | 2178.021 | 4090.1 | 1088.1 | |
| I-901 | 38 | | | | 2164.005 | 1083.1 | 1081.1 | |
| I-902 | 104 | 21.2 | | | 2312.939 | 1157.5 | 1155.4 | |
| I-903 | 33 | 7.8 | | | 2226.979 | 1114.6 | 1112.6 | |
| I-904 | 54 | 20.0 | | | 2252.995 | 1127.6 | 1125.6 | |
| I-905 | 8 | 31.6 | | | 2171.97 | 1087.1 | 1085 | |
| I-906 | 8 | 26.5 | | | 2185.001 | 1093.7 | 1091.6 | |
| I-907 | 9 | 27.8 | | | 2253.064 | 2254.2 | 2252.1 | |
| I-908 | 10 | 26.0 | | | 2261.033 | 2262.1 | 2260.2 | |
| I-909 | 8 | 28.4 | | | 2248.001 | 2249.1 | 2247.4 | |
| I-910 | 10 | 29.8 | | | 2205.99 | 1104.1 | 1102.1 | |
| I-911 | 12 | 22.9 | | | 2174.969 | 1088.6 | 1086.6 | |
| I-912 | 10 | 28.0 | | | 2263.022 | 1132.7 | 1130.6 | |
| I-913 | 10 | 26.4 | | | 2351.074 | 1176.7 | 1174.6 | |
| I-914 | 16 | 38.1 | | | 2439.127 | 1220.7 | 1218.7 | |
| I-915 | 28 | 15.5 | | | 2198.948 | 1107.6 | 1105.6 | |
| I-916 | 28 | 13.6 | | | 2198.948 | 1107.6 | 1105.6 | |
| I-917 | 12 | 19.7 | | | 2114.948 | 1058.6 | 1056.6 | |
| I-918 | 33 | 11.1 | | | 2171.011 | 1086.7 | 1084.6 | |
| I-919 | 28 | 15.2 | | | 2207.011 | 1104.7 | 1102.6 | |
| I-920 | 117 | 17.7 | | | 2249.058 | 1125.7 | 1123.6 | |
| I-921 | 8 | | | | 2055.846 | 1029.2 | 1027.3 | |
| I-922 | 12 | 2.7 | | | 2042.85 | 1022.7 | 1020.6 | |
| I-923 | 27 | 6.0 | | | 2088.871 | 1045.7 | 1043.8 | |
| I-924 | 7 | 3.3 | | | 2099.795 | 1051.5 | 1049.6 | |
| I-925 | 21 | 20.0 | | | 2055.846 | 1029.3 | 1027.2 | |
| I-926 | 43 | 29.9 | | | 2055.846 | 1029.2 | 1027.3 | |
| I-927 | 11 | 8.2 | | | 2089.872 | 1046 | 1044 | |
| I-928 | 21 | 6.1 | | | 2089.872 | 1046 | 1044.1 | |
| I-929 | 23 | 10.3 | | | 2012.876 | 1007.7 | 1005.7 | |
| I-930 | 37 | 13.5 | | | 1999.881 | 1001.3 | 999.3 | |
| I-931 | 221 | | | | 2045.902 | 1024 | 1022 | |
| I-932 | 35 | 11.4 | | | 2056.826 | 1030 | 1027.9 | |
| I-933 | 168 | 22.2 | | | 2012.876 | 1007.8 | 1005.6 | |
| I-934 | 69 | 19.2 | | | 2046.902 | 1024.6 | 1022.6 | |
| I-935 | 8 | 33.1 | | | 2154.943 | 1078.6 | 1076.5 | |
| I-936 | 7 | 22.2 | | | 2198.969 | 1100.6 | 1098.6 | |
| I-937 | 9 | 28.8 | | | 2242.996 | 1122.6 | 1120.6 | |
| I-938 | 9 | 33.5 | | | 2287.022 | 1144.6 | 1142.6 | |
| I-939 | 8 | 25.1 | | | 2044.906 | 1023.6 | 1021.7 | |
| I-940 | 1742 | | | | 1986.814 | 1987.7 | 1985.7 | |
| I-941 | 2111 | | | | 1986.814 | 1987.8 | 1985.8 | |
| I-942 | 1285 | | | | 2000.83 | 2001.7 | 1999.8 | |
| I-943 | 1994 | | | | 2000.83 | 2001.8 | 1999.8 | |
| I-944 | 761 | | | | 1970.819 | 1971.8 | 1969.8 | |
| I-945 | 1079 | | | | 1970.819 | 986.5 | 984.5 | |
| I-946 | 798 | | | | 2012.866 | 2013.9 | 2011.9 | |
| I-947 | 1053 | | | | 2012.866 | 1007.5 | 1005.5 | |
| I-948 | 2433 | | | | 2000.83 | 2001.8 | 1999.7 | |
| I-949 | 2256 | | | | 2000.83 | 2001.8 | 1999.8 | |
| I-950 | 1634 | | | | 2000.83 | 2001.7 | 1999.7 | |
| I-951 | 1471 | | | | 2000.83 | 2001.8 | 1999.8 | |
| I-952 | 1214 | | | | 2062.846 | 2063.8 | 2061.8 | |
| I-953 | 1456 | | | | 2062.846 | 1032.6 | 1030.5 | |
| I-954 | 900 | | | | 1959.803 | 981.1 | 979 | |
| I-955 | 1198 | | | | 1959.803 | 981 | 979 | |
| I-956 | 1086 | | | | 1973.819 | 988 | 986 | |
| I-957 | 1426 | | | | 1973.819 | 988 | 986 | |
| I-958 | 606 | | | | 1973.819 | 988.1 | 986 | |
| I-959 | 881 | | | | 1973.819 | 988 | 986 | |
| I-960 | 336 | | | | 1985.856 | 994 | 992 | |
| I-961 | 357 | | | | 1985.856 | 994.1 | 992.1 | |
| I-962 | 407 | | | | 2019.84 | 1011.1 | 1009 | |
| I-963 | 402 | | | | 2019.84 | 1011.1 | 1009 | |
| I-964 | 1435 | | | | 1973.869 | 988.1 | 986.1 | |
| I-965 | 1780 | | | | 1973.869 | 988.1 | 986.1 | |
| I-966 | 2023 | | | | 1946.858 | 974.6 | 972.5 | |
| I-967 | 1735 | | | | 1946.858 | 974.6 | 972.5 | |
| I-968 | 2981 | | | | 1960.874 | 981.6 | 979.5 | |
| I-969 | 2318 | | | | 1960.874 | 981.7 | 979.5 | |
| I-970 | 1364 | | | | 2006.895 | 1004.6 | 1002.5 | |
| I-971 | 1842 | | | | 2006.895 | 1004.7 | 1002.6 | |
| I-972 | 1819 | | | | 1972.91 | 987.6 | 985.7 | |
| I-973 | 9 | 19.0 | 10.1 | | 1972.91 | 987.7 | 985.6 | |
| I-974 | 1780 | | | | 1905.832 | 954.1 | 952 | |
| I-975 | 2236.5 | | | | 1905.832 | 954.1 | 952 | |
| I-976 | 399 | | | | 1992.843 | 997.6 | 995.5 | |
| I-977 | 875.5 | | | | 1992.843 | 997.6 | 995.6 | |
| I-978 | 112 | | | | 1965.832 | 984.1 | 982.1 | |
| I-979 | 240 | | | | 1965.832 | 984.1 | 982 | |
| I-980 | 221 | | | | 1979.847 | 991.1 | 989 | |
| I-981 | 373.5 | | | | 1979.847 | 991.1 | 989 | |
| I-982 | 190 | | | | 1984.842 | 993.6 | 991.7 | |
| I-983 | 118 | | | | 1984.842 | 993.6 | 991.5 | |
| I-984 | 179 | | | | 1924.805 | 963.5 | 961.5 | |
| I-985 | 430.5 | | | | 1924.805 | 963.6 | 961.4 | |
| I-986 | 136 | | | | 1950.857 | 976.6 | 974.7 | |
| I-987 | 112 | | | | 1950.857 | 976.6 | 974.5 | |
| I-988 | 5 | 13.9 | | | 2054.91 | 2055.7 | 2053.6 | |
| I-989 | 87 | | | | 2070.905 | 1036.5 | 1034.4 | |
| I-990 | 41 | 8.8 | | | 2047.844 | 1025.2 | 1023.1 | |
| I-991 | 51 | | | | 2061.86 | 1032.2 | 1030.2 | |
| I-992 | 59 | | | | 2061.86 | 1032.2 | 1030.2 | |
| I-993 | 18 | 4.9 | 1.8 | 3.6 | 2015.839 | 1009.2 | 1007.4 | |
| I-994 | 121 | | | | 2011.917 | 2013 | 2011 | |
| I-995 | 55 | | | | 1985.865 | 994.2 | 992.1 | |
| I-996 | 21 | 11.3 | 3.6 | | 1999.881 | 1001.2 | 999.2 | |
| I-997 | 154 | | | | 1984.906 | 994 | 992 | |
| I-998 | 127 | | | | 1958.854 | 980.7 | 978.8 | |
| I-999 | 26 | 26.3 | 5.6 | | 1958.854 | 980.7 | 978.6 | |
| I-1000 | 18 | 23.9 | 1.9 | | 1972.87 | 987.7 | 985.5 | |
| I-1001 | 7218 | | | | 2027.841 | 1015 | 1013 | |
| I-1002 | 9 | 29.8 | | | 2097.892 | 1050.2 | 1048.3 | |
| I-1003 | 38 | | | | 2131.877 | 1067.2 | 1065.2 | |
| I-1004 | 5 | 10.6 | | | 2071.84 | 1037.2 | 1035.1 | |
| I-1005 | 42 | | | | 2097.892 | 1050.2 | 1048.2 | |
| I-1006 | 5 | >>20 | | | 2112.903 | 1057.7 | 1055.6 | |
| I-1007 | 6 | 24.9 | | | 2097.892 | 1050.2 | 1048.2 | |
| I-1008 | 9 | 14.0 | 4.7 | | 2131.877 | 1067.2 | 1065.2 | |
| I-1009 | 11 | 33.3 | | | 2071.84 | 1037.2 | 1035.1 | |
| I-1010 | 8 | 13.3 | | | 2097.892 | 1050.3 | 1048.2 | |
| I-1011 | 5 | 20.8 | | | 2112.903 | 1057.7 | 1055.7 | |
| I-1012 | 60 | 37.9 | | | 2187.976 | 1095.3 | 1093.2 | |
| I-1013 | 8 | 12.4 | | | 1931.821 | 967.2 | 964.9 | |
| I-1014 | 19 | 14.3 | | | 1930.837 | 966.7 | 964.6 | |
| I-1015 | 5 | 8.9 | 5.7 | | 1890.794 | 946.6 | 944.4 | |
| I-1016 | 8 | 21.4 | | | 1889.81 | 946.2 | 944.2 | |
| I-1017 | 5 | 8.9 | | | 1917.805 | 960.2 | 958.2 | |
| I-1018 | 26 | 25.8 | | | 1916.821 | 959.7 | 957.7 | |
| I-1019 | 5 | 9.6 | 7.8 | | 1876.779 | 939.7 | 937.8 | |
| I-1020 | 52 | | | | 1875.794 | 939.2 | 937.2 | |
| I-1021 | 5 | 11.5 | | | 1915.85 | 959 | 957 | |
| I-1022 | 33 | 13.7 | | | 1914.866 | 959.8 | 956.5 | |
| I-1023 | 10 | 15.0 | | | 1874.824 | 938.5 | 936.5 | |
| I-1024 | 43 | | | | 1873.84 | 938 | 935.9 | |
| I-1025 | 5 | 23.6 | | | 1917.795 | 960.1 | 958 | |
| I-1026 | 5 | 16.7 | | | 2028.825 | 1015.6 | 1013.6 | |
| I-1028 | 13 | 8.7 | 3.6 | | 1917.805 | 960.2 | 958.2 | |
| I-1029 | 823 | >>20 | | | 1931.821 | 967.2 | 965.1 | |
| I-1030 | 507 | >>20 | | | 1931.821 | 967.2 | 965.1 | |
| I-1031 | 11 | 8.8 | | | 1931.821 | 967.2 | 965 | |
| I-1032 | 15 | 10.4 | | | 1890.794 | 946.7 | 944.5 | |
| I-1033 | 10 | 8.6 | | | 1904.81 | 953.7 | 951.7 | |
| I-1034 | 27 | 9.5 | 4.0 | | 1950.831 | 976.7 | 974.6 | |
| I-1035 | 21 | 10.2 | | | 1916.846 | 959.7 | 957.5 | |
| I-1036 | 29 | 16.6 | | | 1904.81 | 953.7 | 951.7 | |
| I-1037 | 30 | 4.4 | | | 1918.825 | 960.7 | 958.7 | |
| I-1038 | 101 | | | | 1964.846 | 983.7 | 981.6 | |
| I-1039 | 57 | | | | 1930.862 | 966.7 | 964.5 | |
| I-1040 | 8 | 9.3 | | | 1970.832 | 986.7 | 984.8 | |
| I-1041 | 8 | >>20 | | | 1964.785 | 983.7 | 981.6 | |
| I-1042 | 5 | 11.4 | | | 1957.836 | 980.2 | 978.2 | |
| I-1043 | 12 | >>20 | | | 1951.789 | 977.2 | 975.2 | |
| I-1044 | 5 | 17.6 | 14.5 | | 1943.821 | 973.2 | 971.3 | |
| I-1045 | 5 | 23.9 | | | 1937.774 | 970.2 | 968.2 | |
| I-1046 | 5 | 20.5 | | | 1929.805 | 966.2 | 964.1 | |
| I-1047 | 5 | 28.4 | | | 1923.758 | 963.2 | 961 | |
| I-1048 | 5 | 8.8 | | | 1916.81 | 959.7 | 957.6 | |
| I-1049 | 6 | | | | 1910.763 | 956.6 | 954.5 | |
| I-1050 | 5 | 24.8 | | | 1902.794 | 952.7 | 950.5 | |
| I-1051 | 14 | >>20 | | | 1896.747 | 949.6 | 947.5 | |
| I-1061 | 6 | 10.3 | | | 2141.955 | 1072.5 | 1070.2 | |
| I-1062 | 11 | 4.7 | | | 1943.857 | 973.3 | 971.2 | |
| I-1063 | 471 | | | | 1957.873 | 911.7 | 909.7 | |
| I-1064 | 414 | | | | 1902.831 | 952.7 | 950.5 | |
| I-1065 | 520 | | | | 1902.831 | 952.6 | 950.5 | |
| I-1066 | 789 | | | | 1916.846 | 959.7 | 957.7 | |
| I-1067 | 517 | | | | 1916.846 | 959.7 | 957.5 | |
| I-1068 | 950 | | | | 1916.846 | 959.7 | 957.4 | |
| I-1069 | 920 | | | | 1916.846 | 959.7 | 957.8 | |
| I-1070 | 1022 | | | | 1930.862 | 966.7 | 964.7 | |
| I-1071 | 1261 | | | | 1930.862 | 966.8 | 964.7 | |
| I-1072 | 10 | 10.5 | | | 1909.896 | 956 | 953.8 | |
| I-1073 | 409 | | | | 1923.912 | 963.1 | 961 | |
| I-1074 | 498 | | | | 1923.912 | 963.1 | 961 | |
| I-1075 | 567 | | | | 1969.873 | 986.2 | 984.4 | |
| I-1076 | 448 | | | | 1969.873 | 986.3 | 984.2 | |
| I-1077 | 324 | | | | 1983.888 | 993.3 | 991.3 | |
| I-1078 | 352 | | | | 1983.888 | 993.3 | 991.4 | |
| I-1079 | 5 | 19.7 | | | 1972.847 | 987.8 | 985.8 | |
| I-1080 | 6 | >>20 | 10.4 | | 1916.846 | 959.8 | 957.7 | |
| I-1081 | 5 | 16.7 | 10.6 | | 1874.799 | 1876.3 | 1874.2 | |
| I-1082 | 5 | 14.1 | | | 1904.81 | 1906.3 | 1904.2 | |
| I-1083 | 8 | 18.8 | | | 1950.831 | 976.9 | 975 | |
| I-1084 | 368 | | | | 1900.815 | 951.9 | 949.8 | |
| I-1086 | 12 | 10.1 | | | 1888.851 | 1890.3 | 1888.3 | |
| I-1087 | 9 | 9.3 | | | 1888.851 | 1890.3 | 1888.4 | |
| I-1088 | 22 | 6.3 | | | 1902.867 | 1904.3 | 1902.4 | |
| I-1089 | 12 | 8.9 | | | 1902.867 | 1904.3 | 1902.5 | |
| I-1090 | 18 | 6.1 | | | 1861.84 | 1863 | 1861 | |
| I-1091 | 27 | 11.4 | | | 1875.856 | 1877 | 1875 | |
| I-1092 | 26 | 7.7 | | | 1861.84 | 1863 | 1861 | |
| I-1093 | 43 | | | | 1875.856 | 1877 | 1875 | |
| I-1094 | 46 | | | | 1875.856 | 1877 | 1875 | |
| I-1095 | 15 | >>20 | | | 1900.888 | 1902 | 1900 | |
| I-1096 | 17 | 8.6 | | | 1914.903 | 1916.4 | 1914.4 | |
| I-1097 | 40 | >>20 | | | 1873.877 | 1875 | 1873 | |
| I-1098 | 100 | | | | 1887.892 | 945 | 943 | |
| I-1099 | 9 | 9.0 | | | 1842.817 | 922.6 | 920.5 | |
| I-1100 | 16 | 24.3 | | | 1868.833 | 935.6 | 1868.2 | |
| I-1101 | 14 | 27.0 | | | 1860.808 | 931.6 | 929.6 | |
| I-1102 | 16 | 27.2 | | | 1886.824 | 944.6 | 942.6 | |
| I-1103 | 15 | 16.7 | | | 1872.828 | 937.6 | 935.6 | |
| I-1104 | 10 | 17.7 | | | 1898.844 | 950.6 | 948.7 | |
| I-1105 | 36 | >>20 | | | 1848.864 | 925.7 | 923.6 | |
| I-1106 | 23 | >>20 | | | 1848.864 | 925.6 | 923.6 | |
| I-1107 | 18 | 11.8 | | | 2009.904 | 1006.4 | 1004.5 | |
| I-1108 | 18 | >>20 | | | 1987.789 | 995.3 | 993.3 | |
| I-1109 | 24 | >>20 | | | 1987.789 | 995.4 | 993.4 | |
| I-1110 | 38 | >>20 | | | 2013.841 | 1008.4 | 1006.1 | |
| I-1111 | 28 | >>20 | | | 2013.841 | 1008.5 | 1006.1 | |
| I-1112 | 40 | | | | 1928.846 | 965.9 | 963.8 | |
| I-1113 | 64 | | | | 1968.877 | 986 | 984 | |
| I-1114 | 20 | >>20 | | | 1960.779 | 981.8 | 979.9 | |
| I-1115 | 28 | 24.3 | | | 1960.779 | 981.8 | 979.9 | |
| I-1116 | 40 | | | | 1972.815 | 987.9 | 986 | |
| I-1117 | 55 | | | | 1972.815 | 987.9 | 985.8 | |
| I-1118 | 24 | 29.5 | | | 1973.831 | 988.4 | 986.3 | |
| I-1119 | 26 | 19.4 | | | 2011.808 | 1007.3 | 1005.3 | |
| I-1120 | 85 | >>20 | | | 2011.808 | 1007.4 | 1005.3 | |
| I-1121 | 18 | 9.2 | | | 1973.868 | 988.4 | 986.1 | |
| I-1122 | 34 | 12.3 | | | 2011.844 | 1007.4 | 1005.4 | |
| I-1123 | 64 | 28.5 | | | 2011.844 | 1007.4 | 1005.5 | |
| I-1124 | 12 | >>20 | | | 1939.87 | 971.2 | 969.2 | |
| I-1125 | 16 | >>20 | | | 1977.847 | 990.2 | 988.2 | |
| I-1126 | 32 | 26.8 | | | 1977.847 | 990.2 | 988.2 | |
| I-1127 | 18 | 5.4 | | | 1939.907 | 971.2 | 969.2 | |
| I-1128 | 20 | 8.1 | | | 1977.883 | 990.2 | 988.2 | |
| I-1129 | 39 | | | | 1977.883 | 990.2 | 988.1 | |
| I-1130 | 6 | 7.6 | | | 1986.899 | 1988.7 | 1986.7 | |
| I-1131 | 7 | 18.8 | | | 2043.921 | 2045.7 | 2043.8 | |
| I-1132 | 21 | >>20 | | | 2043.921 | 2045.7 | 2043.5 | |
| I-1133 | 5 | | | | 1972.847 | 1974.8 | 1973.4 | |
| I-1134 | 6 | | | | 1972.847 | 1974.8 | 1972.6 | |
| I-1135 | 20 | | | | 1916.81 | 959.8 | | |
| I-1136 | 16 | 14.9 | | | 1916.81 | 959.8 | 957.7 | |
| I-1137 | 8 | 12.0 | | | 1916.81 | 959.8 | 1916.7 | |
| I-1138 | 18 | 19.8 | | | 1928.846 | 1953.4 | 1929.5 | |
| I-1139 | 22 | 18.0 | | | 1942.862 | 972.9 | 1942.8 | |
| I-1140 | 33 | 32.5 | | | 1942.862 | 972.9 | 1943.5 | |
| I-1141 | 13 | 27.3 | | | 1910.763 | 956.8 | 1911.3 | |
| I-1142 | 22 | >>20 | | | 1910.763 | 956.8 | 1911.3 | |
| I-1143 | 12 | 33.1 | | | 1910.763 | 956.7 | 1911.4 | |
| I-1144 | 28 | 32.8 | | | 1922.799 | 962.9 | 1922.9 | |
| I-1145 | 18 | 30.6 | | | 1936.815 | 969.8 | 1936.5 | |
| I-1146 | 33 | >>20 | | | 1936.815 | 969.8 | 1935.6 | |
| I-1147 | 10 | 21.1 | | | 1985.868 | 994.3 | 1986.5 | |
| I-1148 | 50 | 21.5 | | | 1985.868 | 994.2 | 1986.3 | |
| I-1149 | 70 | | | | 2027.915 | 1015.4 | 2027.7 | |
| I-1150 | 12 | 21.4 | | | 1958.857 | 980.8 | 978.6 | |
| I-1151 | 30 | 25.3 | | | 1958.857 | 980.8 | 978.9 | |
| I-1152 | 65 | 14.2 | | | 2000.904 | 2003 | 2001 | |
| I-1153 | 12 | >>20 | | | 1979.821 | 991.3 | 989.1 | |
| I-1154 | 45 | | | | 1979.821 | 991.3 | 989.4 | |
| I-1155 | 60 | | | | 2021.868 | 1012.3 | 1010.6 | |
| I-1156 | 14 | 26.1 | | | 1952.81 | 977.8 | 975.9 | |
| I-1157 | 36 | >>20 | | | 1952.81 | 977.8 | 1953.2 | |
| I-1158 | 168 | | | | 1994.857 | 998.9 | 1995.2 | |
| I-1159 | 61 | | | | 1930.827 | 1932.2 | 1930.3 | |
| I-1160 | 575 | | | | 1930.827 | 1932.3 | 1930.4 | |
| I-1161 | 24 | >>20 | | | 2041.857 | 2043.2 | 2041.3 | |
| I-1162 | 1534 | | | | 2041.857 | 2061.4 | | |
| I-1163 | 691 | | | | 1887.858 | 1889.2 | 1887.3 | |
| I-1164 | 162 | | | | 1887.858 | 1889.2 | 1887.2 | |
| I-1165 | 722 | | | | 1887.858 | 1907.2 | | |
| I-1166 | 14 | 14.6 | | | 1889.837 | 1891.1 | 1889.2 | |
| I-1167 | 34 | 11.7 | | | 1901.873 | 1903.2 | 1901.2 | |
| I-1168 | 25 | 31.4 | | | 1917.841 | 960.3 | 958.3 | |
| I-1169 | 159 | | | | 1929.878 | 1931.6 | 1929.6 | |
| I-1170 | 15 | 13.5 | | | 1848.81 | 925.6 | 923.7 | |
| I-1171 | 70 | 28.8 | | | 1860.847 | 931.6 | 1860.2 | |
| I-1172 | 45 | 19.4 | | | 1888.851 | 939.8 | 937.7 | |
| I-1173 | 12 | 17.2 | | | 1915.852 | 959.2 | 1915.2 | |
| I-1174 | 43 | 32.6 | | | 1927.889 | 965.2 | 1927.2 | |
| I-1175 | 24 | 27.3 | | | 1943.857 | 973.3 | 971.5 | |
| I-1176 | 169 | | | | 1955.893 | 979.2 | 977.3 | |
| I-1177 | 25 | 4.3 | | | 1929.868 | 1931.4 | 1929.4 | |
| I-1178 | 54.5 | 28.8 | | | 1929.868 | 1931.3 | 1929.4 | |
| I-1179 | 346 | | | | 1943.884 | 1945.3 | 1943.4 | |
| I-1180 | 87 | | | | 1943.884 | 1945.2 | 1943.3 | |
| I-1181 | 297 | >>20 | | | 1849.806 | 1851.2 | 1849.3 | |
| I-1182 | 144.5 | | | | 1850.764 | 1852 | 1850.1 | |
| I-1183 | 409 | | | | 1878.769 | 940.8 | 938.8 | |
| I-1184 | 175.5 | | | | 1850.763 | 926.7 | 924.8 | |
| I-1185 | 203 | 26.9 | | | 1999.849 | 1001.1 | 999.1 | |
| I-1186 | 127.5 | 23.6 | | | 2000.808 | 1001.6 | 999.6 | |
| I-1187 | 80.5 | 28.6 | | | 1972.802 | 987.6 | 985.5 | |
| I-1188 | 22.5 | 7.2 | | | 1872.883 | 1874.1 | 1872.2 | |
| I-1189 | 94.5 | | | | 1859.861 | 931 | 1860 | |
| I-1190 | 58.5 | | | | 1831.857 | 917 | 1832 | |
| I-1191 | 38.5 | | | | 1845.872 | 924 | 1845 | |
| I-1192 | 81.5 | | | | 1910.883 | 957 | 955 | |
| I-1193 | 78.5 | | | | 1882.879 | 1884 | 1882 | |
| I-1194 | 66.5 | | | | 1909.888 | 1911.6 | 1909.6 | |
| I-1195 | 29.5 | 16.2 | | | 1881.883 | 1883.5 | 1881.2 | |
| I-1196 | 85 | | | | 1873.877 | 1898 | 1874 | |
| I-1197 | 44 | | | | 1845.872 | 1869 | 1845 | |
| I-1198 | 65 | | | | 1900.888 | 952 | 950 | |
| I-1199 | 44.5 | | | | 1872.883 | 1874 | 1872 | |
| I-1200 | 99 | >>20 | | | 1940.848 | 971.7 | 969.6 | |
| I-1201 | 13 | 19.6 | | | 1940.848 | 971.7 | 969.7 | |
| I-1202 | 16 | 16.3 | | | 1940.848 | 971.7 | 969.6 | |
| I-1203 | 16 | 10.0 | | | 1934.891 | 1936.3 | 1934.4 | |
| I-1204 | 81 | | | | 1934.891 | 1936.2 | 1934.2 | |
| I-1205 | 987 | | | | 1934.891 | 1936.2 | 1934.2 | |
| I-1206 | 32 | 27.8 | | | 1915.827 | 959.2 | 957.1 | |
| I-1207 | 40 | 7.5 | | | 1914.868 | 958.7 | 1914.3 | |
| I-1208 | 349 | | | | 1888.816 | 945.7 | 1988.1 | |
| I-1209 | 479 | | | | 1887.858 | 1889.2 | 1887.3 | |
| I-1210 | 56 | | | | 1929.843 | 966.2 | 1929.2 | |
| I-1211 | 115 | | | | 1928.884 | 1930.2 | 1928.3 | |
| I-1212 | 8663 | | | | 1914.868 | 1916.2 | 1914.2 | |
| I-1213 | 10000 | | | | 1913.91 | 1915.3 | 1913.3 | |
| I-1214 | 116 | | | | 1902.832 | 1904.2 | 1902.2 | |
| I-1215 | 216 | | | | 1901.873 | 1903.2 | 1901.2 | |
| I-1216 | 10000 | | | | 1901.848 | 1903.4 | 1901.3 | |
| I-1217 | 10000 | | | | 1900.889 | 1902.3 | 1900.2 | |
| I-1218 | 10 | >>20 | | | 1952.778 | 1954.2 | 1952.2 | |
| I-1219 | 7 | >>20 | | | 2063.808 | 2065.2 | 2063.3 | |
| I-1220 | 11 | 17.3 | | | 1915.827 | 1917.1 | 1915.2 | |
| I-1221 | 14 | 23.4 | | | 1914.868 | 1916.3 | 1914.4 | |
| I-1222 | 511 | | | | 1888.816 | 945.7 | 943.7 | |
| I-1223 | 550 | | | | 1887.858 | 1889.3 | 1887.3 | |
| I-1224 | 389 | | | | 1929.843 | 1931.1 | 1929.1 | |
| I-1225 | 305 | | | | 1928.884 | 1930.3 | 1928.4 | |
| I-1226 | 559 | | | | 1914.868 | 958.8 | 956.7 | |
| I-1227 | 10000 | | | | 1913.91 | 1915.3 | 1913.3 | |
| I-1228 | 100 | | | | 1902.832 | 1904.2 | 1902.2 | |
| I-1229 | 134 | | | | 1901.873 | 1903.2 | 1901.3 | |
| I-1230 | 10000 | | | | 1901.848 | 952.3 | 950.3 | |
| I-1231 | 10000 | | | | 1900.889 | 1902.3 | 1900.2 | |
| I-1232 | 31 | 43.3 | | | 1897.86 | 1899.2 | 1897.2 | |
| I-1233 | 24 | 54.5 | | | 1897.86 | 1899.1 | 1897.2 | |
| I-1234 | 41 | 23.9 | | | 1909.896 | 1911.2 | 1909.3 | |
| I-1235 | 27 | 24.8 | | | 1909.896 | 1911.2 | 1909.3 | |
| I-1236 | 69 | 20.9 | | | 1937.901 | 1939.7 | 1937.7 | |
| I-1237 | 31 | 39.9 | | | 1937.901 | 1939.6 | 1937.6 | |
| I-1238 | 44 | 23.1 | | | 1868.87 | 935.7 | 1868.2 | |
| I-1239 | 33 | 31.8 | | | 1868.87 | 935.7 | 1868.2 | |
| I-1240 | 33 | 11.5 | | | 2070.882 | 1037 | 1035 | |
| I-1241 | 19 | 15.7 | | | 2002.844 | 1002.9 | 1000.9 | |
| I-1242 | 30 | >>20 | 3.8 | | 1972.834 | 987.9 | 986 | |
| I-1243 | 82 | | | | 2014.881 | 2017 | 2015 | |
| I-1244 | 18 | 17.7 | 15.9 | | 1918.825 | 960.9 | 959 | |
| I-1245 | 9 | 12.2 | 4.1 | | 1891.815 | 947.4 | 945.8 | |
| I-1246 | 13 | 24.3 | 16.2 | | 1861.804 | 932.4 | 1862.5 | |
| I-1247 | 33 | >>20 | | | 1903.851 | 953.4 | 951.5 | |
| I-1248 | 19 | 29.7 | | | 1971.888 | 987.5 | 985.5 | |
| I-1249 | 17 | 25.2 | | | 1903.851 | 953.4 | 951.3 | |
| I-1250 | 31 | >>20 | | | 1873.84 | 938 | 936 | |
| I-1251 | 1940 | | | | 1915.887 | 957 | 959 | |
| I-1252 | 546 | | | | 2001.899 | 1002.5 | 1000.3 | |
| I-1253 | 14 | >>20 | | | 2005.857 | 1004.5 | 1002.4 | |
| I-1254 | 530 | | | | 1972.945 | 988 | 986 | |
| I-1255 | 32 | >>20 | | | 1976.904 | 989.9 | 987.7 | |
| I-1256 | 1466 | | | | 1903.789 | 932 | 930 | |
| I-1257 | 11 | | | | 2006.858 | 2008.3 | 2006.3 | |
| I-1258 | 17 | >>20 | | | 1953.772 | 978.4 | 976.4 | |
| I-1259 | 10 | 18.1 | | | 1961.831 | 982.4 | 980.3 | |
| I-1260 | 23 | 24.0 | | | 1924.818 | 964 | 961.9 | |
| I-1261 | 31 | 19.8 | | | 1932.877 | 967.9 | 965.9 | |
| I-1262 | 1124 | | | | 1889.872 | 946.4 | 944.3 | |
| I-1263 | 43 | 20.0 | | | 1842.854 | 922.7 | 920.6 | |
| I-1264 | 157 | >>20 | | | 1884.901 | 943.8 | 941.6 | |
| I-1265 | 152 | 22.7 | | | 1884.901 | 943.7 | 941.8 | |
| I-1266 | 755 | >>20 | | | 1892.87 | 947.7 | 945.6 | |
| I-1267 | 181 | 36.9 | | | 1892.87 | 947.7 | 945.8 | |
| I-1268 | 198 | 28.4 | | | 1910.841 | 956.7 | 954.7 | |
| I-1269 | 173 | 17.6 | | | 1892.87 | 947.7 | 945.8 | |
| I-1270 | 3699 | | | | 1892.87 | 947.7 | 945.7 | |
| I-1271 | 682 | | | | 1910.841 | 956.7 | 954.5 | |
| I-1272 | 331 | | | | 1836.897 | 919.7 | 917.7 | |
| I-1273 | 9 | >>20 | | | 2003.915 | 1003.6 | 1001.4 | |
| I-1274 | 13 | >>20 | | | 2015.951 | 2018.1 | 2016.4 | |
| I-1275 | 9 | 27.8 | | | 2042.962 | 2045 | 2042.9 | |
| I-1276 | 10 | >>20 | | | 2015.951 | 2018.1 | 2015.9 | |
| I-1277 | 17 | >>20 | | | 2027.987 | 2029.9 | 2027.9 | |
| I-1278 | 10 | 18.0 | | | 2000.915 | 2003.1 | 2001.6 | |
| I-1279 | 12 | 22.9 | | | 1973.904 | 1976 | 1974.5 | |
| I-1280 | 22 | 24.7 | | | 1985.94 | 1988.1 | 1985.9 | |
| I-1281 | 10 | 22.2 | | | 2014.931 | 2017 | 2015.5 | |
| I-1282 | 34 | 43.1 | | | 1987.92 | 995.6 | 993.6 | |
| I-1283 | 26 | 38.6 | | | 1999.956 | 2002.1 | 1999.9 | |
| I-1284 | 10000 | | | | 1894.933 | 1896.5 | 1894.6 | |
| I-1285 | 10000 | | | | 1894.933 | 1896.4 | 1894.5 | |
| I-1286 | 10000 | | | | 1838.87 | 1840.3 | 1838.4 | |
| I-1287 | 10000 | | | | 1838.87 | 1840.3 | 1838.4 | |
| I-1288 | 1164 | | | | 1994.038 | 1995.7 | 1993.6 | |
| I-1289 | 10000 | | | | 1994.038 | 1995.5 | 1993.4 | |
| I-1290 | 10000 | | | | 1888.976 | 1890.3 | 1888.4 | |
| I-1291 | 10000 | | | | 1888.976 | 1890.2 | 1888.3 | |
| I-1292 | 10000 | | | | 1888.976 | 1890.3 | 1888.4 | |
| I-1293 | 10000 | | | | 1867.922 | 935.2 | 1867.3 | |
| I-1294 | 10000 | | | | 1894.933 | 1896.3 | 1894.3 | |
| I-1295 | 10000 | | | | 1838.87 | 1840.2 | 1838.2 | |
| I-1296 | 3785 | | | | 1838.87 | 1840.2 | 1838.3 | |
| I-1297 | 10000 | | | | 1994.038 | 1995.6 | 1993.4 | |
| I-1298 | 10000 | | | | 1888.976 | 1890.3 | 1888.3 | |
| I-1299 | 10000 | | | | 1867.922 | 935.2 | 1837.3 | |
| I-1300 | 10000 | | | | 1867.922 | 935.2 | 1867.3 | |
| I-1301 | 10000 | | | | 1893.949 | 1895.2 | 1893.3 | |
| I-1302 | 45 | 21.4 | 4.0 | | 1875.846 | 1877.2 | 1875.3 | |
| I-1303 | 86 | 9.1 | | | 1909.831 | 1911.2 | 1909.2 | |
| I-1304 | 31 | 33.6 | | | 1874.862 | 1876.3 | 1874.3 | |
| I-1305 | 6 | 9.7 | | | 1863.81 | 1865.2 | 1863.2 | |
| I-1306 | 6 | 6.6 | | | 1863.81 | 1865.2 | 1863.2 | |
| I-1307 | 21 | 38.8 | | | 1929.867 | 1932 | 1930 | |
| I-1308 | 25 | 11.9 | | | 1848.845 | 1851 | 1849 | |
| I-1309 | 59 | 28.6 | | | 1877.835 | 1879.6 | 1877.6 | |
| I-1310 | 124 | >>20 | | | 1903.851 | 953.4 | 951.4 | |
| I-1311 | 38 | 20.6 | | | 1937.835 | 1939.6 | 1937.5 | |
| I-1312 | 14 | 16.1 | | | 1917.83 | 960.3 | 958.7 | |
| I-1313 | 12 | 51.4 | | | 2002.956 | 2005.1 | 2003.6 | |
| I-1314 | 9 | >>20 | | | 1953.861 | 1955.2 | 1953.2 | |
| I-1315 | 13 | 7.2 | | | 1952.902 | 1954.3 | 1952.3 | |
| I-1316 | 11 | 6.8 | | | 1986.886 | 1988.3 | 1986.3 | |
| I-1317 | 9 | 9.7 | | | 1978.842 | 1980.2 | 1978.2 | |
| I-1318 | 14 | 4.8 | 3.9 | | 1977.883 | 1979.1 | 1977.2 | |
| I-1319 | 10 | 4.2 | 2.1 | | 2011.868 | 2013.2 | 2011.3 | |
| I-1320 | 10 | 26.2 | | | 1928.846 | 1929.2 | 1927.2 | |
| I-1321 | 8 | 10.9 | | | 1927.887 | 1930.2 | 1928.3 | |
| I-1322 | 11 | 9.1 | | | 1961.871 | 1963.2 | 1961.2 | |
| I-1323 | 6 | 11.7 | | | 1988.765 | 1991.1 | 1989.2 | |
| I-1324 | 14 | 5.8 | | | 1987.807 | 1990.5 | 1988.6 | |
| I-1325 | 14 | 5.7 | | | 2021.791 | 2024.5 | 2022.7 | |
| I-1326 | 12 | 6.2 | | | 1957.896 | 1959.5 | 1957.6 | |
| I-1327 | 13 | 7.0 | | | 1939.907 | 1941.4 | 1939.6 | |
| I-1328 | 11 | 5.4 | | | 1973.891 | 1975.5 | 1973.5 | |
| I-1329 | 23 | 35.0 | | | 1916.902 | 1918.4 | 1916.5 | |
| I-1330 | 104 | | | | 1915.943 | 1917.5 | 1915.6 | |
| I-1331 | 75 | | | | 1949.927 | 1951.4 | 1949.5 | |
| I-1332 | 11 | 2.5 | 0.9 | | 2019.888 | 1011.5 | 1009.2 | |
| I-1333 | 20 | 2.8 | 1.2 | | 1978.862 | 991 | 1979.1 | |
| I-1334 | 30 | 4.2 | 2.2 | | 1979.882 | 991.4 | 1980.7 | |
| I-1335 | 11 | 2.2 | 0.9 | >20 | 2053.915 | 1028.3 | 1026.2 | |
| I-1336 | 29 | 2.7 | 0.7 | | 2012.888 | 1007.7 | 1005.8 | |
| I-1337 | 85 | 6.2 | | | 2013.909 | 1008.3 | 1006.6 | |
| I-1338 | 10 | 3.6 | 3.4 | | 2053.873 | 1028.5 | 1026.7 | |
| I-1339 | 33 | 5.2 | | | 2012.846 | 1008 | 1006.2 | |
| I-1340 | 159 | 6.8 | | | 2013.867 | 1008.5 | 1005.9 | |
| I-1341 | 17 | 3.5 | 3.2 | | 2087.899 | 1045.3 | 1043.6 | |
| I-1342 | 35 | 7.0 | | | 2046.873 | 1024.8 | 2046.5 | |
| I-1343 | 150 | | | | 2047.893 | 2049.6 | 2047.2 | |
| I-1344 | 11 | 4.1 | | | 1915.852 | 1917.4 | 1915.4 | |
| I-1345 | 15 | 4.7 | | | 1949.837 | 1951.4 | 1949.5 | |
| I-1346 | 2319 | | | | 1910.855 | 1912.4 | 1910.5 | |
| I-1347 | 2369 | | | | 1909.896 | 1911.4 | 1909.4 | |
| I-1348 | 1181 | | | | 1943.88 | 1945.4 | 1943.5 | |
| I-1349 | 161 | | | | 1924.871 | 1926.4 | 1924.4 | |
| I-1350 | 158 | | | | 1923.912 | 1925.4 | 1923.4 | |
| I-1351 | 258 | | | | 1957.896 | 1959.4 | 1957.5 | |
| I-1352 | 12 | 19.5 | | | 1902.886 | 1904.4 | 1902.5 | |
| I-1353 | 18 | 8.8 | | | 1901.927 | 1903.4 | 1901.4 | |
| I-1354 | 48 | | | | 1913.891 | 1915.3 | 1912.7 | |
| I-1355 | 213 | | | | 1926.911 | 964.7 | 1926.4 | |
| I-1356 | 23 | >>20 | | | 1905.828 | 954.2 | 1905.4 | |
| I-1357 | 10 | 33.6 | | | 1886.844 | 944.7 | 1886.5 | |
| I-1358 | 19 | | | | 1980.97 | 1982.4 | 1980.5 | |
| I-1359 | 88 | | | | 1980.97 | 1982.4 | 1980.4 | |
| I-1360 | 135 | | | | 1993.99 | 998 | 996 | |
| I-1361 | 39 | >>20 | | | 1972.907 | 1974.4 | 1972.5 | |
| I-1362 | 23 | 19.6 | | | 1953.922 | 1955.4 | 1953.5 | |
| I-1363 | 9 | 5.5 | | | 2017.824 | 1010.2 | 2017.6 | |
| I-1364 | 9 | 10.5 | | | 2017.824 | 2019.4 | 2017.5 | |
| I-1365 | 9 | 9.7 | | | 2017.824 | 2019.4 | 2017.5 | |
| I-1366 | 7 | 7.5 | | | 1977.841 | 1979.8 | 1977.9 | |
| I-1367 | 31 | 7.8 | | | 2045.829 | 2047.8 | 2045.9 | |
| I-1368 | 44 | 14.2 | | | 2045.829 | 2047.8 | 2046.6 | |
| I-1369 | 29 | 9.9 | | | 2045.829 | 2047.8 | 2045.9 | |
| I-1370 | 18 | 10.0 | | | 1977.818 | 1979.4 | 1977.5 | |
| I-1371 | 22 | 10.9 | | | 1977.818 | 1979.4 | 1977.5 | |
| I-1372 | 29 | 10.2 | | | 1977.818 | 1979.4 | 1977.6 | |
| I-1373 | 5 | 18.3 | | | 1972.874 | 1974.6 | 1972.5 | |
| I-1374 | 17 | 8.8 | | | 1998.89 | 2000.5 | 1998.5 | |
| I-1375 | 7 | 18.5 | | | 1956.843 | 1958.5 | 1956.4 | |
| I-1376 | 4208 | | | | 1937.975 | 1939.7 | 1937.6 | |
| I-1377 | 1003 | | | | 1938.011 | 1939.6 | 1937.6 | |
| I-1378 | 6171 | | | | 1910.964 | 1912.7 | 1910.6 | |
| I-1379 | 1435 | | | | 1964.986 | 1966.7 | 1964.6 | |
| I-1380 | 10000 | | | | 1965.022 | 1966.6 | 1964.6 | |
| I-1381 | 10000 | | | | 1932.019 | 1933.7 | 1931.6 | |
| I-1382 | 2853 | | | | 1937.975 | 1939.6 | 1937.6 | |
| I-1383 | 1655 | | | | 1938.011 | 1939.6 | 1937.7 | |
| I-1384 | 10000 | | | | 1910.964 | 1912.5 | 1910.5 | |
| I-1385 | 1082 | | | | 1964.986 | 1966.8 | 1964.6 | |
| I-1386 | 2248 | | | | 1965.022 | 1966.6 | 1964.6 | |
| I-1387 | 6938 | | | | 1932.019 | 1933.6 | 1931.6 | |
| I-1388 | 18 | 11.3 | | | 1854.89 | 1856.7 | 1854.8 | |
| I-1389 | 9 | 9.2 | | | 1860.847 | 1862.6 | 1860.7 | |
| I-1390 | 16 | 6.6 | | | 1820.841 | 1822.6 | 1820.6 | |
| I-1391 | 11 | 35.0 | | | 1873.842 | 1875.5 | 1873.7 | |
| I-1392 | 13 | 18.3 | | | 1860.847 | 1862.5 | 1860.6 | |
| I-1393 | 34 | 18.6 | | | 1820.841 | 1822.5 | 1820.7 | |
| I-1394 | 9 | 15.9 | | | 1901.847 | 1903.8 | 1901.8 | |
| I-1395 | 33 | 15.4 | | | 1888.851 | 945.9 | 943.9 | |
| I-1396 | 33 | 15.3 | | | 1935.873 | 1937.4 | 1935.5 | |
| I-1397 | 48 | 16.4 | | | 1922.878 | 1924.4 | 1922.5 | |
| I-1398 | 73 | | | | 1882.871 | 1884 | 1882 | |
| I-1399 | 13 | 17.4 | | | 1901.837 | 1903.4 | 1901.6 | |
| I-1400 | 5 | 20.3 | | | 1901.837 | 1903.4 | 1901.5 | |
| I-1401 | 5 | 21.4 | | | 1901.837 | 1903.4 | 1901.5 | |
| I-1402 | 9 | 27.1 | | | 1895.88 | 1897.5 | 1895.5 | |
| I-1403 | 6 | 29.1 | | | 1895.88 | 1897.4 | 1895.5 | |
| I-1404 | 113 | 36.8 | | | 1895.88 | 1897.4 | 1895.5 | |
| I-1405 | 9 | 40.1 | | | 1895.88 | 1897.4 | 1895.5 | |
| I-1406 | 6 | 5.0 | | | 1977.868 | 1979.4 | 1977.5 | |
| I-1407 | 5 | 4.2 | | | 1977.868 | 1979.5 | 1977.6 | |
| I-1408 | 33 | 6.0 | | | 1977.868 | 1979.4 | 1977.6 | |
| I-1409 | 74 | 24.0 | | | 1900.878 | 952 | 1901 | |
| I-1410 | 80 | 29.3 | | | 1886.862 | 945 | 1987 | |
| I-1411 | 109 | 47.1 | | | 1886.862 | 945 | 1987 | |
| I-1412 | 79 | >>20 | | | 1886.862 | 945 | 1987 | |
| I-1413 | 74 | | | | 1887.821 | 945.3 | 943.3 | |
| I-1414 | 84 | >>20 | | | 1860.81 | 931.8 | 929.8 | |
| I-1415 | 72 | | | | 1874.826 | 938.8 | 1874.4 | |
| I-1416 | 57 | | | | 1928.883 | 966 | 1929 | |
| I-1417 | 198 | | | | 1928.883 | 1953 | 1929 | |
| I-1418 | 106 | >>20 | | | 1914.867 | 959 | 1916 | |
| I-1419 | 152 | | | | 1914.867 | 959 | 1916 | |
| I-1420 | 124 | | | | 1914.867 | 959 | 1916 | |
| I-1421 | 134 | | | | 1914.867 | 1939 | 1916 | |
| I-1422 | 388 | | | | 1915.826 | 959.6 | 957.3 | |
| I-1423 | 389 | | | | 1888.815 | 946.1 | 1889.7 | |
| I-1424 | 235 | | | | 1902.831 | 953 | 1902.9 | |
| I-1425 | 239 | | | | 1895.862 | 1897.4 | 1895.5 | |
| I-1426 | 203 | | | | 1854.835 | 928.7 | 1854.4 | |
| I-1427 | 33 | 30.4 | | | 1895.862 | 949.2 | 1895.4 | |
| I-1428 | 41 | 40.2 | | | 1854.835 | 928.7 | 1854.5 | |
| I-1429 | 10000 | | | | 1919.902 | 961.3 | 1919.5 | |
| I-1430 | 10000 | | | | 1878.875 | 940.7 | 1878.4 | |
| I-1431 | 44 | 14.6 | | | 1898.891 | 1900.4 | 1898.5 | |
| I-1432 | 27 | 14.5 | | | 1857.865 | 930.2 | 1857.4 | |
| I-1433 | 71 | | | | 1912.907 | 1914.4 | 1912.5 | |
| I-1434 | 83 | | | | 1871.88 | 937.2 | 1871.5 | |
| I-1435 | 90 | | | | 1932.852 | 967.9 | 965.9 | |
| I-1436 | 121 | | | | 1891.826 | 947.4 | 945.6 | |
| I-1437 | 7 | 7.1 | | | 1929.868 | 1931.5 | 1929.6 | |
| I-1438 | 5 | 7.4 | | | 1943.884 | 1945.5 | 1943.5 | |
| I-1439 | 349 | >>20 | | | 1986.905 | 1988.6 | 1986.6 | |
| I-1440 | 26 | 20.7 | | | 1928.884 | 1930.4 | 1928.5 | |
| I-1441 | 327 | | | | 1947.894 | 1949.5 | 1947.6 | |
| I-1442 | 1500 | | | | 1899.894 | 1901.5 | 1899.6 | |
| I-1443 | 24 | 9.5 | | | 1887.858 | 945.3 | 1887.8 | |
| I-1444 | 6558 | | | | 1913.91 | 1945.6 | 1913.8 | |
| I-1445 | 13 | 11.1 | | | 2044.91 | 2046.7 | 2044.8 | |
| I-1446 | 9 | 35.2 | | | 2083.877 | 2085.8 | 2083.9 | |
| I-1447 | 11 | 43.4 | | | 2063.889 | 2065.9 | 2064 | |
| I-1448 | 20 | | | | 2153.842 | 2155.8 | 2153.9 | |
| I-1449 | 16 | 29.3 | | | 2153.842 | 2155.8 | 2153.9 | |
| I-1450 | 11 | | | | 2153.842 | 2155.8 | 2154 | |
| I-1451 | 13 | 26.2 | | | 2015.889 | 2018 | 2016.1 | |
| I-1452 | 66 | | | | 1944.915 | 1946.6 | 1944.6 | |
| I-1453 | 52 | | | | 1944.915 | 1946.6 | 1944.5 | |
| I-1454 | 22 | 26.8 | | | 2012.903 | 2014.5 | 2012.6 | |
| I-1455 | 13 | 23.1 | | | 2012.903 | 2014.5 | 2012.6 | |
| I-1456 | 16 | 23.7 | | | 1992.915 | 1994.4 | 1992.6 | |
| I-1457 | 43 | >>20 | | | 2011.856 | 2013.8 | 2011.8 | |
| I-1458 | 26 | 18.1 | | | 1881.901 | | | |
| I-1459 | 5 | | | | 1939.827 | | | |
| I-1460 | 7 | | | | 1939.827 | | | |
| I-1461 | 10 | 3.7 | 1.7 | | 2026.882 | | | |
| I-1462 | 8 | 2.3 | 2.7 | 8.4 | 1991.884 | | | |
| I-1463 | 18 | | | | 2042.85 | | | |
| I-1464 | 168 | | | | 1948.847 | 1950.8 | 1948.9 | A |
| I-1465 | 10000 | | | | 1994.038 | 1996.1 | 1994 | |
| I-1466 | 10 | 2.2 | 0.7 | | 2053.915 | | | |
| I-1467 | 12 | 9.6 | | | 2066.985 | 1033.8 | 2065.1 | |
| I-1468 | 233 | | | | 1990.954 | 1990.4 | 1988.6 | |
| I-1469 | 95 | | | | 2275.135 | 2276.8 | 2274.9 | |
| I-1470 | 57 | >>20 | | | 2420.209 | | | |
| I-1471 | 34 | >>20 | | | 2120.999 | 2122.6 | 2120.7 | |
| I-1472 | 12 | >>20 | | | 2266.073 | 2267.7 | 2265.8 | |
| I-1473 | 224 | | | | 2331.198 | 1167 | 1165 | |
| I-1474 | 41 | >>20 | | | 2476.271 | 2478 | 2476.1 | |
| I-1475 | 113 | | | | 2177.062 | 2178.7 | 2176.9 | |
| I-1476 | 16 | >>20 | | | 2322.136 | 2324 | 2322.2 | |
| I-1477 | 148 | | | | 2402.235 | 2404 | 2402 | |
| I-1478 | 75 | 17.1 | | | 2547.309 | 1275 | 1273 | |
| I-1479 | 173 | | | | 2248.099 | 2250 | 2248 | |
| I-1480 | 37 | 9.3 | | | 2393.173 | 2394.9 | 2393 | |
| I-1481 | 7 | 39.7 | | | 2028.937 | 2030.7 | 2028.6 | |
| I-1482 | 7 | 18.0 | | | 2062.938 | 2064.7 | 2062.6 | |
| I-1483 | 22 | >>20 | | | 2028.9 | 2030.5 | 2028.5 | |
| I-1484 | 30 | >>20 | | | 2041.932 | 2043.7 | 2041.6 | |
| I-1485 | 7 | 25.8 | | | 1986.89 | 1988.8 | 1986.5 | |
| I-1486 | 5 | 17.9 | | | 2020.891 | 1988.4 | 1986.5 | |
| I-1487 | 7 | 21.5 | | | 1986.853 | 2022.7 | 2020.6 | |
| I-1488 | 6 | 30.3 | | | 1999.885 | 2001.6 | 1999.5 | |
| I-1489 | 17 | 9.2 | | | 2062.954 | 2066.6 | 2064.6 | |
| I-1490 | 226 | | | | 1986.923 | 1990.3 | 1987.4 | |
| I-1491 | 34 | | | | 1848.845 | 1873 | 1849 | |
| I-1492 | 5 | 7.3 | | | 1901.837 | 1903.5 | 1901.6 | |
| I-1493 | 10 | 4.9 | | | 1874.826 | 1898.5 | 1874.6 | |
| I-1494 | 5 | 14.7 | | | 1888.842 | 1912.4 | 1988.5 | |
| I-1495 | 24 | 23.9 | | | 1886.862 | 1910.4 | 1886.5 | |
| I-1496 | 51 | 9.4 | | | 1934.862 | 1958.4 | 1934.5 | |
| I-1497 | 50 | 11.4 | | | 1973.873 | 1997.4 | 1973.5 | |
| I-1498 | 48 | | | | 1925.848 | 1949.4 | 1925.4 | |
| I-1499 | 23 | 6.4 | | | 1888.842 | 1912.3 | 1888.4 | |
| I-1500 | 22 | 4.7 | | | 1915.852 | 1917.3 | 1915.4 | |
| I-1501 | 46 | >>20 | | | 2028.937 | 2030.6 | 2028.6 | |
| I-1502 | 34 | >>20 | | | 2062.938 | 2064.7 | 2062.5 | |
| I-1503 | 38 | | | | 2041.968 | 2043.6 | 2041.6 | |
| I-1504 | 46 | >>20 | | | 2014.921 | 2016.6 | 2014.5 | |
| I-1505 | 39 | >>20 | | | 2048.923 | 2050.6 | 2048.7 | |
| I-1506 | 89 | | | | 2027.953 | 2029.6 | 2027.6 | |
| I-1507 | 30 | >>20 | | | 2028.9 | 2030.4 | 2028.5 | |
| I-1508 | 87 | | | | 2041.932 | 2043.6 | 2041.6 | |
| I-1509 | 11 | 24.5 | | | 1935.821 | | | |
| I-1510 | 10 | 31.8 | | | 1908.81 | | | |
| I-1511 | 5 | >>20 | | | 1951.816 | | | |
| I-1512 | 12 | >>20 | | | 1924.805 | | | |
| I-1513 | 21 | >>20 | | | 1875.785 | | | |
| I-1514 | 27 | | | | 1848.774 | | | |
| I-1515 | 24 | >>20 | | | 1889.8 | | | |
| I-1516 | 30 | >>20 | | | 1862.79 | | | |
| I-1517 | 32 | >>20 | | | 1902.796 | | | |
| I-1518 | 23 | 24.2 | | | 1875.785 | | | |
| I-1519 | 10 | 12.8 | | | 1941.868 | | | |
| I-1520 | 11 | 17.4 | | | 1914.857 | | | |
| I-1521 | 16 | 6.0 | | | 1881.901 | 1883.4 | 1881.4 | |
| I-1522 | 6 | 5.8 | | | 1881.901 | 1883.3 | 1881.4 | |
| I-1523 | 29 | 8.0 | | | 1866.927 | 1868.5 | 1866.6 | |
| I-1524 | 17 | 8.1 | | | 1854.89 | 1856.3 | 1854.4 | |
| I-1525 | 22 | 10.8 | | | 1854.89 | 1856.4 | 1854.5 | |
| I-1526 | 84 | >>20 | | | 1855.849 | 1857.3 | 1855.4 | |
| I-1527 | 36 | 30.3 | | | 1855.849 | 1857.3 | 1855.4 | |
| I-1528 | 102 | 31.4 | | | 1840.875 | 1842.4 | 1840.5 | |
| I-1529 | 126 | >>20 | | | 1828.838 | 1830.3 | 1828.4 | |
| I-1530 | 61 | 36.9 | | | 1828.838 | 1830.4 | 1828.4 | |
| I-1531 | 84 | >>20 | | | 1882.86 | 1884.3 | 1882.4 | |
| I-1532 | 49 | >>20 | | | 1882.86 | 1884.4 | 1882.4 | |
| I-1533 | 76 | >>20 | | | 1867.885 | 1891.3 | 1867.4 | |
| I-1534 | 181 | >>20 | | | 1855.849 | 1879.3 | 1855.4 | |
| I-1535 | 72 | >>20 | | | 1855.849 | 1879.3 | 1855.4 | |
| I-1536 | 42 | 18.4 | | | 1880.942 | 1882.4 | 1880.5 | |
| I-1537 | 31 | 13.9 | | | 1868.906 | 1870.4 | 1868.5 | |
| I-1538 | 21 | 11.8 | | | 1868.906 | 1870.4 | 1868.4 | |
| I-1539 | 36 | >>20 | | | 1896.876 | 1898.4 | 1896.4 | |
| I-1540 | 17 | 42.4 | | | 1896.876 | 1898.4 | 1896.5 | |
| I-1541 | 39 | 34.7 | | | 1881.901 | 1883.4 | 1881.5 | |
| I-1542 | 64 | 49.1 | | | 1869.865 | 1871.4 | 1869.4 | |
| I-1543 | 21 | >>20 | | | 1869.865 | 1871.3 | 1869.4 | |
| I-1544 | 61 | >>20 | | | 1869.865 | 1871.3 | 1869.4 | |
| I-1545 | 26 | >>20 | | | 1869.865 | 1871.3 | 1869.4 | |
| I-1546 | 71 | 37.9 | | | 1854.89 | 1856.4 | 1854.5 | |
| I-1547 | 68 | >>20 | | | 1842.854 | 1844.4 | 1842.5 | |
| I-1548 | 28 | >>20 | | | 1842.854 | 1844.3 | 1842.4 | |
| I-1549 | 13 | 11.6 | | | 1915.885 | 1917.4 | 1915.5 | |
| I-1550 | 22 | 15.3 | | | 1900.911 | 1902.4 | 1900.6 | |
| I-1551 | 16 | 19.8 | | | 1888.875 | 1890.3 | 1888.4 | |
| I-1552 | 56 | >>20 | | | 1896.912 | | | |
| I-1553 | 133 | >>20 | | | 1881.938 | | | |
| I-1554 | 63 | >>20 | | | 1869.901 | | | |
| I-1555 | 180 | 29.3 | | | 1914.843 | | | |
| I-1556 | 385 | >>20 | | | 1858.781 | | | |
| I-1557 | 342 | >>20 | | | 1913.884 | | | |
| I-1558 | 678 | | | | 1942.875 | | | |
| I-1559 | 9683 | | | | 1901.848 | | | |
| I-1560 | 368 | | | | 1928.859 | | | |
| I-1561 | 2604 | | | | 1887.832 | | | |
| I-1562 | 5020 | | | | 1913.884 | | | |
| I-1563 | 2847 | | | | 1913.884 | | | |
| I-1564 | 122 | >>20 | | | 1901.848 | | | |
| I-1565 | 148 | >>20 | | | 1901.848 | | | |
| I-1566 | 231 | >>20 | | | 1928.859 | | | |
| I-1567 | 182 | >>20 | | | 1928.859 | | | |
| I-1568 | 7181 | | | | 1963.864 | | | |
| I-1569 | 2662 | | | | 1963.864 | | | |
| I-1570 | 1343 | | | | 1913.884 | 1915.4 | 1913.5 | |
| I-1571 | 15 | 11.7 | | | 2119.858 | 2121.8 | 2119.9 | |
| I-1572 | 20 | >>20 | | | 1973.842 | | | |
| I-1573 | 16 | >>20 | | | 1974.817 | 1976.3 | 1974.5 | |
| I-1574 | 15 | >>20 | | | 2010.784 | 2012.3 | 2010.5 | |
| I-1575 | 79 | | | | 1960.801 | 1962.3 | 1960.4 | |
| I-1576 | 224 | | | | 1938.763 | 1940.3 | 1938.4 | |
| I-1577 | 45 | | | | 1938.763 | 1940.3 | 1938.4 | |
| I-1578 | 11 | >>20 | | | 2016.865 | 1009.7 | 1007.8 | |
| I-1579 | 39 | >>20 | | | 2016.865 | 1009.7 | 1007.8 | |
| I-1580 | 1427 | | | | 2014.006 | | | |
| I-1581 | 10000 | | | | 2053.017 | | | |
| I-1582 | 1223 | | | | 1980.022 | | | |
| I-1583 | 3421 | | | | 1953.97 | | | |
| I-1584 | 2436 | | | | 1994.996 | | | |
| I-1585 | 10000 | | | | 1954.06 | | | |
| I-1586 | 10000 | | | | 2011.037 | | | |
| I-1587 | 10000 | | | | 2011.037 | | | |
| I-1588 | 10000 | | | | 1995.067 | | | |
| I-1589 | 10000 | | | | 1991.092 | | | |
| I-1590 | 7520.5 | | | | 1993.071 | | | |
| I-1591 | 3114.5 | | | | 2037.08 | | | |
| I-1592 | 2850 | | | | 2083.064 | 2085.1 | 2083.1 | |
| I-1593 | 10000 | | | | 2040.058 | 2041.9 | 2039.9 | |
| I-1594 | 10000 | | | | 2020.09 | 2021.9 | 2019.9 | |
| I-1595 | 10000 | | | | 2108.046 | 2109.8 | 2107.9 | |
| I-1596 | 10000 | | | | 1964.027 | 1965.8 | 1963.7 | |
| I-1597 | 6989 | | | | 1934.001 | 1935.7 | 1933.7 | |
| I-1598 | 10000 | | | | 1974.069 | 1975.7 | 1973.7 | |
| I-1599 | 1075 | | | | 2028.022 | 2029.8 | 2027.8 | |
| I-1600 | 4290 | | | | 2067.033 | 2068.8 | 2066.7 | |
| I-1601 | 2018 | | | | 1967.986 | 1969.7 | 1967.6 | |
| I-1602 | 362 | >>20 | | | 1994.996 | 1996.7 | 1994.7 | |
| I-1603 | 10000 | | | | 1974.069 | 1975.7 | 1973.7 | |
| I-1604 | 123 | | | | 2057.946 | 2059.8 | 2057.7 | A |
| I-1605 | 214 | | | | 2030.913 | 2032.5 | 2030.6 | A |
| I-1606 | 107 | | | | 2046.882 | 2032.5 | 2030.6 | A |
| I-1607 | 49 | 11.9 | | | 2132.915 | 2135.1 | 2133 | |
| I-1608 | 76 | 15.1 | | | 2143.979 | 2145.8 | 2143.6 | |
| I-1609 | 15 | 6.7 | | | 2098.913 | 2101.2 | 2099.7 | |
| I-1610 | 19 | 4.1 | | | 2109.977 | 2111.9 | 2109.6 | |
| I-1611 | 10 | 9.8 | | | 2111.908 | 2114.1 | 2112 | |
| I-1612 | 10 | 5.3 | 4.0 | | 2122.973 | 2124.8 | 2122.6 | |
| I-1613 | 9 | 5.2 | | | 2098.876 | 2100.9 | 2098.9 | |
| I-1614 | 15 | 6.4 | | | 2109.941 | 2111.6 | 2109.6 | |
| I-1615 | 9 | 8.0 | | | 2084.934 | 2087.3 | 2085.2 | |
| I-1616 | 13 | 4.5 | 2.8 | | 2095.998 | 2097.9 | 2095.6 | |
| I-1617 | 759 | | | | 1858.879 | | | |
| I-1618 | 10000 | | | | 1900.889 | 1902.4 | 1900.5 | |
| I-1619 | 1292 | | | | 1900.889 | 1902.4 | 1900.5 | |
| I-1620 | 2057 | | | | 1914.905 | 1916.4 | 1914.6 | |
| I-1621 | 266 | | | | 1888.853 | 1890.4 | 1888.5 | |
| I-1622 | 13 | >>20 | | | 2001.874 | | | |
| I-1623 | 30 | 27.7 | | | 2029.905 | 1016.6 | 1014.7 | |
| I-1624 | 47.5 | 42.9 | | | 2082.868 | | | |
| I-1625 | 52.5 | 29.3 | | | 1998.887 | | | |
| I-1626 | 721 | | | | 1958.931 | 980.9 | 978.9 | |
| I-1627 | 57 | >>20 | | | 1930.9 | 1932.6 | 1930.5 | |
| I-1628 | 18 | >>20 | | | 1930.9 | 1932.6 | 1930.5 | |
| I-1629 | 12 | 4.3 | 2.7 | | 2054.913 | | | |
| I-1630 | 11.5 | 5.0 | | | 2034.926 | | | |
| I-1631 | 10 | 30.1 | | | 1958.895 | 1960.5 | 1958.6 | |
| I-1632 | 10 | 6.5 | 2.6 | | 2124.879 | 2126.4 | 2124.5 | |
| I-1633 | 10 | 5.2 | | | 2040.898 | 2042.4 | 2040.5 | |
| I-1634 | 9 | 16.5 | | | 2055.872 | | | |
| I-1635 | 7 | 20.6 | | | 2035.885 | 2037.5 | 2035.6 | |
| I-1636 | 17 | >>20 | | | 1959.854 | 1961.5 | 1959.5 | |
| I-1637 | 11 | >>20 | | | 1959.854 | 1961.5 | 1959.5 | |
| I-1638 | 9 | 14.5 | | | 2125.838 | 2127.4 | 2125.5 | |
| I-1639 | 13 | 32.9 | | | 2125.838 | 2127.4 | 2125.5 | |
| I-1640 | 8 | | | | 2041.857 | 2043.4 | 2041.5 | |
| I-1641 | | 14.7 | | | 2083.904 | 2085.5 | 2083.7 | |
| I-1642 | 36 | | | | 2109.919 | 2111.5 | 2109.6 | |
| I-1643 | 822 | | | | 2021.085 | 2022.7 | 2020.7 | |
| I-1644 | 10000 | | | | 2020.09 | 2021.8 | 2019.7 | |
| I-1645 | 940 | | | | 2063.132 | 2064.8 | 2062.7 | |
| I-1646 | 640 | | | | 1994.038 | 1995.8 | 1993.6 | |
| I-1647 | 2354 | | | | 2057.012 | 2058.8 | 2056.6 | |
| I-1648 | 191 | >>20 | | | 2047.064 | 2048.8 | 2046.7 | |
| I-1649 | 2482 | | | | 2027.095 | 2028.8 | 2026.7 | |
| I-1650 | 1683 | | | | 2061.08 | 2062.9 | 2060.7 | |
| I-1651 | 3968 | | | | 2027.095 | 2028.9 | 2026.7 | |
| I-1652 | 10000 | | | | 2046.105 | 2047.9 | 2045.8 | |
| I-1653 | 903 | | | | 2078.106 | 2079.7 | 2077.7 | |
| I-1654 | 2128 | | | | 2120.19 | 2121.8 | 2119.9 | |
| I-1655 | 48 | 12.7 | | | 2087.899 | 2089.7 | 2087.8 | |
| I-1656 | 20 | 10.9 | | | 2053.915 | 2055.6 | 2053.6 | |
| I-1657 | 36 | 11.0 | | | 2053.873 | 2056 | 2054.1 | |
| I-1658 | 19 | 9.8 | | | 2019.888 | 2022.1 | 2020 | |
| I-1659 | 108 | 11.4 | | | 2046.873 | 2070.4 | 2046.6 | |
| I-1660 | 99 | | | | 2012.888 | 2036.4 | 2012.6 | |
| I-1661 | 70 | 8.3 | | | 2012.888 | 2036.4 | 2012.5 | |
| I-1662 | 69 | 12.0 | | | 2012.846 | 2036.8 | 2013 | |
| I-1663 | 67 | 8.5 | | | 1978.862 | 2002.8 | 1979.7 | |
| I-1664 | 22 | 8.0 | | | 1984.842 | 2008.4 | 1984.6 | |
| I-1665 | 17 | 9.7 | | | 1984.842 | 993.8 | 991.8 | |
| I-1666 | 15 | 6.5 | | | 1950.857 | 976.7 | 967.7 | |
| I-1667 | 13 | 5.6 | 1.3 | | 1950.857 | 976.8 | 974.8 | |
| I-1668 | 29 | 16.4 | | | 1985.862 | 994.3 | 992.3 | |
| I-1669 | 30 | 11.8 | | | 1985.862 | 1987.5 | 1985.7 | |
| I-1670 | 11 | 7.9 | | | 1951.878 | 1953.4 | 1951.6 | |
| I-1671 | 28 | 8.0 | 1.7 | | 1951.878 | 1953.5 | 1951.6 | |
| I-1672 | 40 | 7.1 | | | 2047.92 | | | |
| I-1673 | 28 | | | | 2077.93 | 2080 | 2078.1 | |
| I-1674 | 147 | 18.3 | | | 2153.962 | 2156 | 2154.1 | |
| I-1675 | 18.5 | 9.7 | | | 2103.946 | 2106 | 2104.1 | |
| I-1676 | 91 | | | | 2119.977 | 2121.9 | 2120.1 | |
| I-1677 | 17 | | | | 2107.941 | 2110 | 2108 | |
| I-1678 | 32 | 9.7 | | | 2054.913 | 1028.8 | 1026.9 | |
| I-1679 | 17.5 | | | | 2084.924 | 2086.6 | 2084.7 | |
| I-1680 | 80.5 | 12.5 | | | 2160.955 | 2162.6 | 2160.7 | |
| I-1681 | 18.5 | 5.4 | | | 2110.94 | 2112.5 | 2110.7 | |
| I-1682 | 41 | | | | 2126.971 | 2128.5 | 2126.6 | |
| I-1683 | 9 | 3.7 | | | 2114.934 | 2116.7 | 2114.9 | |
| I-1684 | 13 | 4.5 | 3.4 | | 2005.873 | 2008 | 2006.5 | |
| I-1685 | 23 | 3.5 | 1.3 | | 2061.935 | 1032.5 | 1030.3 | |
| I-1686 | 11 | 5.4 | 0.9/8.7 | | 2033.904 | 2035.9 | 2034.1 | |
| I-1687 | 42 | 18.6 | | | 2005.873 | 1004.4 | 1002.2 | |
| I-1688 | 59 | 21.5 | | | 2047.92 | 1025.5 | 1023.7 | |
| I-1689 | 25.5 | 12.5 | | | 2019.888 | 1011.5 | 1009.5 | |
| I-1690 | 11 | | | | 2028.835 | 1015.9 | 1013.9 | |
| I-1691 | 11 | 12.8 | | | 2028.835 | 1015.9 | 1013.9 | |
| I-1692 | 17 | 10.4 | | | 2084.897 | 1044 | 1042.2 | |
| I-1693 | 10 | 4.6 | | | 2056.866 | 1029.9 | 1027.7 | |
| I-1694 | 57 | 43.6 | | | 2028.835 | 2030.8 | 2028.8 | |
| I-1695 | 93 | >>20 | | | 2070.882 | 2072.9 | 2070.9 | |
| I-1696 | 32 | 35.5 | | | 2042.85 | 1022.9 | 1021 | |
| I-1697 | 179 | >>20 | | | 2044.929 | 2046.7 | 2044.9 | A |
| I-1698 | 165 | >>20 | | | 1987.944 | 2032.6 | 2030.7 | A |
| I-1699 | 533 | | | | 2019.871 | 1011.5 | 1009.4 | A |
| I-1700 | 16 | 4.8 | | | 2020.926 | 2022.6 | 2020.7 | |
| I-1701 | 25 | 24.8 | | | 2021.921 | 2023.2 | 2021.4 | |
| I-1702 | 17 | 6.9 | | | 2045.921 | 2047.5 | 2045.6 | |
| I-1703 | 24 | 6.6 | | | 2012.957 | 1007.8 | 1005.9 | |
| I-1704 | 37 | 5.3 | | | 2088.913 | 2090.3 | 2088.4 | |
| I-1705 | 9 | 2.9 | | | 2012.866 | 2014.3 | 2012.4 | |
| I-1706 | 24 | 4.8 | 2.7 | | 1999.871 | 1001.3 | 999.3 | |
| I-1707 | 19 | 6.1 | | | 2088.898 | 2090.4 | 2088.5 | |
| I-1708 | 29 | 11.9 | | | 2088.898 | 2014.4 | 2012.4 | |
| I-1709 | 14 | 6.2 | 3.9 | | 2068.929 | 1035.8 | 1033.8 | |
| I-1710 | 22 | 3.9 | 1.9 | | 1969.861 | 986.2 | 984.3 | |
| I-1711 | 232 | 33.3 | | | 2024.957 | 2026.4 | 2024.5 | A |
| I-1712 | 597 | | | | 2049.952 | 2051.4 | 2049.5 | A |
| I-1713 | 498 | >>20 | | | 2016.988 | 2018.4 | 2016.5 | A |
| I-1714 | 372 | 28.6 | | | 2092.944 | 2094.4 | 2092.5 | A |
| I-1715 | 95 | 38.4 | | | 2016.898 | 2018.4 | 2016.4 | A |
| I-1716 | 224 | 21.7 | | | 2003.902 | 1003.3 | 1001.3 | A |
| I-1717 | 249 | | | | 2092.929 | 2094.4 | 2092.5 | A |
| I-1718 | 382 | | | | 2092.929 | 1047.8 | 1045.8 | A |
| I-1719 | 187 | 27.6 | | | 2072.96 | 1037.8 | 1035.8 | A |
| I-1720 | 212 | | | | 1973.892 | | | A |
| I-1721 | 139 | >>20 | | | 1960.793 | 981 | | |
| I-1722 | 135 | >>20 | | | 1960.793 | 1961.5 | | |
| I-1723 | 62 | | | | 2113.818 | | | |
| I-1724 | 551 | 29.7 | | | 2113.818 | | | |
| I-1725 | 15 | 10.0 | | | 2034.941 | 2036.3 | 2034.4 | |
| I-1726 | 274 | >>20 | | | 2038.973 | 2040.4 | 2038.4 | A |
| I-1727 | 28 | 34.1 | | | 1959.827 | 1961.8 | | |
| I-1728 | 14 | | | | 1931.822 | 1933.5 | | |
| I-1729 | 9 | 23.0 | | | 1986.89 | 1988.5 | 1986.6 | |
| I-1730 | 38 | | | | 2040.861 | 2042.5 | 2040.6 | |
| I-1731 | 25 | 29.5 | | | 1944.879 | 1946.5 | 1944.5 | |
| I-1732 | 11 | >>20 | | | 1945.838 | 1947.6 | 1945.5 | |
| I-1733 | 15 | >>20 | | | 1945.838 | 1947.5 | 1945.5 | |
| I-1734 | 8 | | | | 2091.853 | 2093.4 | 2091.5 | |
| I-1735 | 667 | | | | 1895.892 | 1897.3 | 1895.3 | |
| I-1736 | 458 | | | | 1901.848 | 1903.3 | 1901.4 | |
| I-1737 | 518 | | | | 1909.907 | 1911.3 | 1909.3 | |
| I-1738 | 1058 | | | | 1963.879 | 1965.3 | 1963.3 | |
| I-1739 | 3655 | | | | 1909.907 | 1911.3 | 1909.3 | |
| I-1740 | 10000 | | | | 1934.874 | | | |
| I-1741 | 4393 | | | | 1973.884 | 1975.3 | 1973.4 | |
| I-1742 | 867 | | | | 1773.789 | | | |
| I-1743 | 469 | | | | 1929.891 | 1931.5 | 1929.4 | |
| I-1744 | 363 | | | | 1920.858 | 961.7 | 959.7 | |
| I-1745 | 402 | | | | 1874.837 | | | |
| I-1746 | 235 | | | | 1887.832 | 1889.4 | 1887.3 | |
| I-1747 | 187 | >>20 | | | 2011.908 | 1007.3 | 1005.3 | |
| I-1748 | 141 | 20.2 | | | 2011.908 | 1007.2 | 1005.3 | |
| I-1749 | 145 | | | | 2045.892 | 1024.2 | 1022.3 | |
| I-1750 | 104 | 23.7 | | | 2045.892 | 1024.2 | 1022.3 | |
| I-1751 | 104 | | | | 1997.892 | | | |
| I-1752 | 87 | | | | 1997.892 | 1000.3 | 998.3 | |
| I-1753 | 802 | | | | 2015.939 | 1009.3 | 1007.3 | A |
| I-1754 | 660 | | | | 2003.902 | 1003.2 | 1001.3 | A |
| I-1755 | 570 | | | | 2049.923 | | | A |
| I-1756 | 329 | | | | 2037.887 | | | A |
| I-1757 | 449 | | | | 2001.923 | | | A |
| I-1758 | 235 | | | | 1989.887 | | | A |
| I-1759 | 57 | >>20 | | | 2111.012 | 2112.6 | 2110.6 | |
| I-1760 | 32 | >>20 | | | 2098.976 | 2100.6 | 2098.5 | |
| I-1761 | 39 | >>20 | | | 2144.997 | 2146.8 | 2144.8 | |
| I-1762 | 13 | >>20 | | | 2132.96 | 2134.7 | 2132.6 | |
| I-1763 | 52 | >>20 | | | 2096.997 | 2098.8 | 2096.8 | |
| I-1764 | 23 | >>20 | | | 2084.96 | 2086.6 | 2084.5 | |
| I-1765 | 667 | | | | 2115.044 | 2116.7 | 2114.7 | A |
| I-1766 | 267 | | | | 2103.007 | 2104.8 | 2102.8 | A |
| I-1767 | 327 | | | | 2149.028 | 2150.7 | 2148.6 | A |
| I-1768 | 69 | >>20 | | | 2136.992 | 2138.7 | 2136.6 | A |
| I-1769 | 419 | | | | 2101.028 | 2012.6 | 2010.6 | A |
| I-1770 | 175 | >>20 | | | 2088.992 | | | A |
| I-1771 | 209 | | | | 2054.913 | | | |
| I-1772 | 14 | 6.8 | | | 2040.898 | 2042.3 | 2040.4 | |
| I-1773 | 32 | 8.6 | | | 2040.898 | 2042.3 | 2040.4 | |
| I-1774 | 28 | 8.9 | | | 2066.913 | 2068.3 | 2066.4 | |
| I-1775 | 15 | | | | 2052.898 | 2054.3 | 2052.4 | |
| I-1776 | 17 | 10.5 | | | 2026.882 | 2028.2 | 2026.3 | |
| I-1777 | 8 | 8.7 | | | 2012.866 | 2014.3 | 2012.4 | |
| I-1778 | 2075 | | | | 1816.82 | 909.7 | 907.7 | |
| I-1779 | | | | | 1816.82 | | | |
| I-1780 | 4192 | | | | 1830.836 | | | |
| I-1781 | 8452 | | | | 1830.836 | | | |
| I-1782 | | | | | 1943.884 | | | |
| I-1783 | 101 | >>20 | | | 1951.852 | 1953.3 | 1951.4 | |
| I-1784 | 528 | | | | 1963.889 | 1965.3 | 1963.4 | |
| I-1785 | 31 | 42.3 | | | 1978.863 | 1980.4 | 1978.5 | |
| I-1786 | 42 | >>20 | | | 1965.868 | 1967.4 | 1965.6 | |
| I-1787 | 776 | | | | 1977.905 | 1979.6 | 1977.8 | |
| I-1788 | 58 | 33.9 | | | 1987.864 | 1989.3 | 1987.4 | |
| I-1789 | 183 | | | | 1974.868 | 1976.3 | 1974.4 | |
| I-1790 | 82 | 34.4 | | | 2038.806 | 2040.5 | 2038.6 | |
| I-1791 | 182 | >>20 | | | 2025.81 | 2027.3 | 2025.4 | |
| I-1792 | 935 | | | | 2037.847 | 2039.3 | 2037.4 | |
| I-1793 | 20 | >>20 | | | 1925.848 | 1927.5 | 1925.4 | |
| I-1794 | 52 | 37.7 | | | 1925.848 | 1927.4 | 1925.4 | |
| I-1795 | 28 | 22.8 | | | 1937.884 | 1939.5 | 1937.4 | |
| I-1796 | 22 | >>20 | | | 1937.884 | 1939.5 | 1937.4 | |
| I-1797 | 10 | 22.5 | | | 2178.018 | 2179.9 | 2177.6 | |
| I-1798 | 12 | | | | 2178.018 | 2179.7 | 2177.6 | |
| I-1799 | 8 | 25.8 | | | 2181.029 | 2182.6 | 2180.6 | |
| I-1800 | 13 | 25.2 | | | 2181.029 | 2182.8 | 2180.9 | |
| I-1801 | 7 | 17.5 | | | 2193.029 | 2191.7 | 2192.6 | |
| I-1802 | 11 | 25.2 | | | 2193.029 | 2194.7 | 2192.6 | |
| I-1803 | 20 | 7.4 | | | 2177.943 | 2179.5 | 2177.6 | |
| I-1804 | 8 | 9.4 | | | 2123.971 | 2125.6 | 2123.5 | |
| I-1805 | 6 | 9.3 | | | 2123.971 | 2125.7 | 2123.7 | |
| I-1806 | 8777 | | | | 1750.774 | 1752.3 | 1750.3 | |
| I-1807 | 163 | >>20 | | | 1750.774 | 1752.4 | 1750.4 | |
| I-1808 | 90 | 14.0 | | | 1919.884 | 1921.4 | 1919.5 | A |
| I-1809 | 1947 | | | | 1752.789 | 1754.4 | 1752.4 | A |
| I-1810 | 416 | | | | 1851.821 | 1853.3 | 1851.4 | A |
| I-1811 | 156 | 32.3 | | | 1878.919 | 1880.3 | 1878.4 | |
| I-1812 | 181 | | | | 1902.796 | 1904.3 | 1902.4 | |
| I-1813 | 26 | >>20 | | | 1902.796 | 1904.3 | 1902.4 | |
| I-1814 | 189 | | | | 1901.837 | 1903.4 | 1901.5 | |
| I-1815 | 46 | >>20 | | | 1901.837 | 1903.4 | 1901.4 | |
| I-1816 | 235 | | | | 1888.78 | 1890.4 | 1888.4 | |
| I-1817 | 182 | | | | 1888.78 | 1890.4 | 1888.4 | |
| I-1818 | 236 | >>20 | | | 1887.821 | 1889.3 | 1887.4 | |
| I-1819 | 615 | | | | 1859.826 | 1861.4 | 1859.4 | |
| I-1820 | 422 | | | | 1858.867 | 1860.4 | 1858.5 | |
| I-1821 | 67 | >>20 | | | 1906.827 | 1908.3 | 1906.4 | A |
| I-1822 | 60 | >>20 | | | 1905.868 | 1907.4 | 1905.5 | A |
| I-1823 | 41 | >>20 | | | 1892.811 | 1894.4 | 1892.5 | A |
| I-1824 | 101 | >>20 | | | 1891.852 | 1893.4 | 1891.4 | A |
| I-1825 | 366 | | | | 1863.858 | 1865.4 | 1863.4 | A |
| I-1826 | 474 | | | | 1874.764 | | | |
| I-1827 | 308 | | | | 1874.764 | 1876.3 | 1874.3 | |
| I-1828 | 264 | >>20 | | | 1873.806 | 983.9 | 982.1 | |
| I-1829 | 668 | | | | 1904.811 | 1906.4 | 1904.4 | |
| I-1830 | 313 | | | | 1903.852 | 1905.4 | 1903.5 | |
| I-1831 | 1635 | | | | 1878.796 | 1880.3 | 1878.3 | A |
| I-1832 | 1110 | | | | 1877.837 | 1879.3 | 1877.4 | A |
| I-1833 | 26 | 6.9 | | | 2054.887 | 2056.9 | 2054.9 | |
| I-1834 | 35 | 18.8 | | | 2048.942 | 2050.6 | 2048.6 | |
| I-1835 | 34 | 31.9 | | | 1986.926 | 1988.5 | 1986.5 | |
| I-1836 | 24 | | | | 2016.9 | 2018.3 | 2016.4 | |
| I-1837 | 19 | 18.5 | | | 2068.929 | 2070.5 | 2068.5 | |
| I-1838 | 85 | 7.9 | | | 2138.895 | 2140.4 | 2138.5 | |
| I-1839 | 21 | 14.6 | | | 2054.913 | 2056.5 | 2054.5 | |
| I-1840 | 14 | 22.3 | | | 2000.905 | 2002.4 | 2000.5 | |
| I-1841 | 76 | >>20 | | | 1958.895 | 1960.5 | 1958.4 | |
| I-1842 | 81 | | | | 2071.979 | 2073.5 | 2071.5 | |
| I-1843 | 32 | 9.5 | | | 2051.88 | 2053.3 | 2051.4 | |
| I-1844 | 227 | | | | 2051.88 | 2055.3 | 2053.5 | |
| I-1845 | 30 | 6.3 | | | 2007.89 | 2009.3 | 2007.4 | |
| I-1846 | 38 | 29.8 | | | 2100.856 | 2102.7 | 2100.8 | |
| I-1847 | 56 | 26.2 | | | 2077.894 | 2079.7 | 2078.1 | |
| I-1848 | 42 | >>20 | | | 2068.841 | 2070.3 | 2068.4 | |
| I-1849 | 19 | 8.8 | | | 1936.815 | 970 | 1937 | |
| I-1850 | 17 | 3.8 | | | 1964.846 | 1875.3 | 1873.4 | |
| I-1851 | 25 | 3.8 | | | 1962.867 | 982.9 | 1963.7 | |
| I-1852 | 48 | 4.9 | | | 1891.83 | 947.4 | 1892.9 | |
| I-1853 | 114 | 16.3 | | | 1977.878 | 990.4 | 1978.6 | |
| I-1854 | | | | | 2074.926 | | | |
| I-1855 | 849 | | | | 2074.926 | 2076.5 | 2074.5 | |
| I-1856 | | | | | 2115.952 | | | |
| I-1857 | 4025 | | | | 2115.952 | 2117.6 | 2115.5 | |
| I-1858 | 1108 | | | | 2116.936 | 2118.4 | 2116.4 | |
| I-1859 | 1106 | | | | 2116.936 | 2118.6 | 2116.5 | |
| I-1860 | 512 | | | | 2164.903 | 1083.8 | 1081.8 | |
| I-1861 | 14 | 37.3 | | | 2108.91 | 2110.4 | 2008.3 | |
| I-1862 | 10 | 19.3 | | | 2108.91 | 2110.4 | 2108.9 | |
| I-1863 | 10 | 37.4 | | | 2149.937 | 2151.6 | 2149.5 | |
| I-1864 | 14 | >>20 | | | 2149.937 | 2151.5 | 2149.5 | |
| I-1865 | 10 | >>20 | | | 2150.921 | 2151.5 | 2149.5 | |
| I-1866 | 26 | >>20 | | | 2150.921 | 1076.4 | 1074.5 | |
| I-1867 | 7 | 16.4 | | | 2150.921 | 2152.5 | 2150.6 | |
| I-1868 | 12 | 39.6 | | | 2198.888 | 2200.5 | 2198.6 | |
| I-1869 | 12 | | | | 2198.888 | 1100.8 | 2198.6 | |
| I-1870 | 1522 | | | | 1895.892 | 1897.4 | 1895.4 | |
| I-1871 | 1765 | | | | 1895.892 | 1897.3 | 1895.4 | |
| I-1872 | 1499 | | | | 1895.892 | 1897.4 | 1895.4 | |
| I-1873 | 1479 | | | | 1895.892 | 1897.3 | 1895.4 | |
| I-1874 | 10000 | | | | 1851.923 | 1853.4 | 1851.4 | |
| I-1875 | 10000 | | | | 1851.923 | 1853.3 | 1851.4 | |
| I-1876 | 1338 | | | | 1884.887 | 1886.3 | 1884.4 | |
| I-1877 | 1524 | | | | 1884.887 | 1886.3 | 1884.3 | |
| I-1878 | 458 | | | | 1872.858 | 937.7 | 1872.4 | |
| I-1879 | 567 | | | | 1872.858 | 937.7 | 1872.4 | |
| I-1880 | 868 | | | | 1886.874 | 944.7 | 1886.4 | |
| I-1881 | 743 | | | | 1886.874 | 944.7 | 1886.4 | |
| I-1882 | 397 | | | | 1943.906 | 1945.7 | 1943.4 | |
| I-1883 | 884 | | | | 1943.906 | 1945.6 | 1943.4 | |
| I-1884 | 249 | | | | 1927.864 | 1929.4 | 1927.4 | |
| I-1885 | 255 | | | | 1927.864 | 965.3 | 1927.5 | |
| I-1886 | 10000 | | | | 1957.886 | 1959.8 | 1957.6 | |
| I-1887 | 10000 | | | | 1957.886 | 1959.6 | 1957.6 | |
| I-1888 | 10000 | | | | 1943.906 | 1945.6 | 1943.5 | |
| I-1889 | 10000 | | | | 1943.906 | 1945.6 | 1943.5 | |
| I-1890 | 31 | >>20 | | | 1957.886 | 1959.7 | 1957.4 | |
| I-1891 | 40 | >>20 | | | 1957.886 | 1959.4 | 1957.6 | |
| I-1892 | 1418 | | | | 1956.901 | 1958.6 | 1956.5 | |
| I-1893 | | | | | 1956.901 | | | |
| I-1894 | | | | | 1970.893 | | | |
| I-1895 | | | | | 1956.877 | | | |
| I-1896 | 75 | | | | 1936.932 | 1938.6 | 1936.4 | |
| I-1897 | | | | | 1922.916 | | | |
| I-1898 | 22 | 10.9 | | | 1922.916 | 962.7 | 1922.5 | |
| I-1899 | 28 | 5.1 | | | 1992.877 | 1994.8 | 1992.7 | |
| I-1900 | 136 | >>20 | | | 1992.877 | 998 | 1993 | |
| I-1901 | 34 | 4.9 | | | 1994.857 | 997.9 | 1993.7 | |
| I-1902 | 28 | 7.7 | | | 1994.857 | 998.9 | 997.1 | |
| I-1903 | 14 | 6.4 | | | 1952.81 | 977.8 | 1953.5 | |
| I-1904 | 15 | 4.2 | | >20 | 2005.873 | 1004.4 | 1002.2 | |
| I-1905 | 20 | 9.7 | | | 2066.914 | 2069.1 | 2067.6 | |
| I-1906 | 23 | 15.5 | | | 2062.919 | 2064.9 | 2063.6 | |
| I-1907 | 23 | | | | 2036.904 | 2039 | 2037.7 | |
| I-1908 | | | | | 2006.893 | | | |
| I-1909 | 29 | >>20 | | | 2109.998 | 2111.8 | 2109.7 | |
| I-1910 | 17 | 13.8 | | | 2076.915 | 2078.6 | 2076.6 | |
| I-1911 | 38 | >>20 | | | 2067.951 | 2069.9 | 2067.9 | |
| I-1912 | 13 | 11.5 | | | 2067.915 | 1035.3 | 2067.6 | |
| I-1913 | 119 | 17.5 | | | 2052.941 | 1027.8 | 2052.7 | |
| I-1914 | 52 | 6.4 | | | 2024.909 | 1016.8 | 2024.7 | |
| I-1915 | 11 | 20.2 | | | 2033.967 | 2035.8 | 2033.8 | |
| I-1916 | 8 | 9.2 | | | 2000.884 | 2002.5 | 2000.5 | |
| I-1917 | 13 | 37.7 | | | 1991.92 | 1993.7 | 1991.6 | |
| I-1918 | 5 | 4.0 | 6.5 | | 1991.884 | 997.3 | 1991.7 | |
| I-1919 | 33 | 4.7 | | | 1976.909 | 989.7 | 1976.5 | |
| I-1920 | 18 | 3.1 | 1.2 | | 1948.878 | 975.7 | 1948.5 | |
| I-1921 | 22 | | | | 1998.926 | | | |
| I-1922 | 457 | | | | 2008.017 | 2009.8 | 2007.7 | |
| I-1923 | 10000 | | | | 2022.007 | 2023.6 | 2021.6 | |
| I-1924 | 1606 | | | | 2054.973 | 2057.2 | 2055.8 | |
| I-1925 | 475 | | | | 2064.018 | 2065.8 | 2063.6 | |
| I-1926 | 417 | | | | 2051.023 | 2052.7 | 2050.6 | |
| I-1927 | 5136 | | | | 2022.007 | 2023.6 | 2021.6 | |
| I-1928 | 887 | | | | 2054.973 | 2009.8 | 2007.7 | |
| I-1929 | 74 | >>20 | | | 2064.018 | 1995.8 | 1993.6 | |
| I-1930 | 1565 | | | | 2051.023 | 2065.7 | 2063.6 | |
| I-1931 | 1558 | | | | 2037.007 | 2052.8 | 2050.6 | |
| I-1932 | 285 | | | | 2008.017 | 2038.7 | 2036.6 | |
| I-1933 | 154 | | | | 1994.001 | | | |
| I-1934 | 5 | | | | 2147.91 | 2149.5 | 2147.4 | |
| I-1935 | 9 | 7.1 | | | 2192.917 | 2194.3 | 2192.4 | |
| I-1936 | 10 | 15.7 | | | 2196.004 | 2197.6 | 2195.5 | |
| I-1937 | 9 | | | | 2208.004 | 2209.6 | 2208.3 | |
| I-1938 | 8 | 30.0 | | | 2187.941 | 2189.6 | 2187.4 | |
| I-1939 | 10 | 20.7 | | | 2138.946 | 2140.5 | 2138.4 | |
| I-1940 | 6 | 15.4 | | | 2192.993 | 2194.6 | 2192.6 | |
| I-1941 | 6 | 12.9 | | | 2110.914 | 2112.5 | 2110.3 | |
| I-1942 | 11 | 7.9 | | | 2131.904 | 2133.3 | 2131.4 | |
| I-1943 | 7 | 3.8 | | | 2111.898 | 2113.2 | 2111.3 | |
| I-1944 | 1634 | | | | 1931.83 | | | |
| I-1945 | 918 | | | | 1931.83 | | | |
| I-1946 | 874 | | | | 1931.83 | | | |
| I-1947 | 253 | | | | 1931.83 | | | |
| I-1948 | 1331 | | | | 1945.845 | | | |
| I-1949 | 96 | 28.5 | | | 1945.845 | | | |
| I-1950 | 234 | | | | 2084.854 | 1043.9 | 2084.6 | |
| I-1951 | 264 | >>20 | | | 2098.87 | 2100.4 | 2098.6 | |
| I-1952 | 923 | >>20 | | | 2083.859 | 1043.4 | 2082.8 | |
| I-1953 | 306 | >>20 | | | 2083.859 | 1043.3 | 2084.3 | |
| I-1954 | 994 | >>20 | | | 2083.859 | 2043.4 | 2041.6 | |
| I-1955 | 1201 | | | | 2083.859 | 2085.4 | 2083.3 | |
| I-1956 | 849 | | | | 2083.859 | 2085.3 | 2083.5 | |
| I-1957 | 567 | | | | 2097.875 | 2099.4 | 2097.4 | |
| I-1958 | 1536 | >>20 | | | 2097.875 | 2099.6 | 2097.5 | |
| I-1959 | 412 | >>20 | | | 2091.922 | | | |
| I-1960 | 544 | 20.4 | | | 2091.922 | | | |
| I-1961 | 2099 | >>20 | | | 2091.922 | | | |
| I-1962 | 1604 | >>20 | | | 2091.922 | | | |
| I-1963 | 326 | 25.6 | | | 2035.859 | 1019.3 | 1017.4 | |
| I-1964 | 738 | | | | 2035.859 | 2037.4 | 2034.6 | |
| I-1965 | 633 | 25.2 | | | 2035.859 | 1019.3 | 2035.6 | |
| I-1966 | 566 | | | | 2049.875 | 2051.5 | 2049.6 | |
| I-1967 | 14 | | | | 1998.926 | | | |
| I-1968 | 29 | | | | 1998.926 | | | |
| I-1969 | 49 | | | | 1998.926 | | | |
| I-1970 | 441 | | | | 1873.806 | 1875.8 | 1873.4 | |
| I-1971 | 475 | | | | 1887.821 | 1889.8 | 1887.7 | |
| I-1972 | 689 | | | | 1887.821 | 1889.9 | 1887.8 | |
| I-1973 | 797 | | | | 1901.837 | 1903.9 | 1902 | |
| I-1974 | 324 | | | | 1877.837 | 1879.8 | 1877.9 | A |
| I-1975 | 745 | | | | 1891.852 | 1891.9 | 1889.9 | A |
| I-1976 | 150 | | | | 1905.868 | 1907.9 | 1905.9 | A |
| I-1977 | 4283 | | | | 1764.789 | 1766.3 | 1764.4 | |
| I-1978 | 145 | >>20 | | | 1764.789 | 1766.3 | 1764.4 | |
| I-1979 | 10000 | | | | 1736.758 | 1738.3 | 1736.3 | |
| I-1980 | 236 | >>20 | | | 1722.742 | 1724.3 | 1722.2 | |
| I-1981 | 567 | | | | 1766.805 | 1766.3 | 1764.4 | A |
| I-1982 | 9663 | | | | 1738.774 | 1738.3 | 1736.4 | A |
| I-1983 | 186 | >>20 | | | 1724.758 | 1724.3 | 1722.3 | A |
| I-1984 | 320 | | | | 2019.888 | 1011.5 | 2020 | |
| I-1985 | 325 | | | | 2033.904 | 1018.5 | 2033.9 | |
| I-1986 | 502 | | | | 2056.866 | 1029.9 | 1028.1 | |
| I-1987 | 54 | | | | 1920.843 | 1922.4 | 1920.6 | A |
| I-1988 | 312 | | | | 1863.821 | 1865.6 | 1863.8 | |
| I-1989 | 186 | | | | 1835.79 | 1837.4 | 1835.6 | |
| I-1990 | 585 | | | | 1821.774 | 1823.4 | 1821.6 | |
| I-1991 | 397 | | | | 1865.837 | 1867.4 | 1865.5 | A |
| I-1992 | 536 | | | | 1837.806 | 1839.4 | 1837.5 | A |
| I-1993 | 728 | | | | 1823.79 | 1925.8 | | A |
| I-1994 | 10000 | | | | 1909.907 | 1911.4 | 1909.6 | |
| I-1995 | 10000 | | | | 1909.907 | 1911.4 | 1909.5 | |
| I-1996 | 10000 | | | | 1963.879 | 1965.4 | 1963.5 | |
| I-1997 | 3394 | | | | 1963.879 | 1965.2 | 1963.2 | |
| I-1998 | 732 | | | | 1929.853 | 1931.6 | 1929.8 | |
| I-1999 | 4937 | | | | 1929.853 | 966.4 | 1930 | |
| I-2000 | 6560 | | | | 1929.853 | 1932 | 1930.3 | |
| I-2001 | 4454 | | | | 1929.853 | 1931.8 | 1929.9 | |
| I-2002 | 612 | | | | 1845.785 | 1847.3 | 1845.4 | |
| I-2003 | 1015 | | | | 1845.785 | 1847.2 | 1845.2 | |
| I-2004 | 227 | | | | 2000.953 | 2002.8 | 2000.7 | |
| I-2005 | 357 | | | | 2000.953 | 2002.7 | 2000.5 | |
| I-2006 | 285 | | | | 2000.953 | 2002.8 | 2000.7 | |
| I-2007 | 10000 | | | | 1915.864 | 1917.5 | 1915.5 | |
| I-2008 | 8825 | | | | 1915.864 | 1917.4 | 1915.5 | |
| I-2009 | 1480 | | | | 1969.835 | 1971.4 | 1969.6 | |
| I-2010 | 1973 | | | | 1969.835 | 1971.4 | 1969.5 | |
| I-2011 | 10000 | | | | 1935.809 | 1937.8 | 1935.9 | |
| I-2012 | 10000 | | | | 1935.809 | 1937.7 | 1935.7 | |
| I-2013 | 661 | | | | 1935.809 | 1939.2 | 1937.3 | |
| I-2014 | 1178 | | | | 1935.809 | 969.8 | 967.4 | |
| I-2015 | 522 | | | | 1917.843 | 1920 | 1917.9 | |
| I-2016 | 703 | | | | 1917.843 | 1920 | 1918.1 | |
| I-2017 | 2054 | | | | 1916.811 | 1918.3 | 1916.4 | |
| I-2018 | 1678 | | | | 1916.811 | 1918.3 | 1916.4 | |
| I-2019 | 1449 | | | | 1915.852 | 1917.4 | 1915.4 | |
| I-2020 | 1201 | | | | 1915.852 | 1917.4 | 1915.5 | |
| I-2021 | 5380 | | | | 1920.843 | 1922.4 | 1920.4 | A |
| I-2022 | 1895 | | | | 1919.884 | 1921.4 | 1919.5 | |
| I-2023 | 32 | 7.8 | | | 1920.82 | 962.3 | 1922.2 | |
| I-2024 | 78 | 17.9 | | | 1948.851 | 976.4 | 974.2 | |
| I-2025 | 109 | | | | 1996.851 | 1000.4 | 998.3 | |
| I-2026 | 26 | 9.7 | | | 1920.82 | 962.3 | 960.3 | |
| I-2027 | 13 | 4.1 | | | 1936.815 | 970.3 | 968.1 | |
| I-2028 | 32 | 17.6 | | | 1964.846 | 984.3 | 982.2 | |
| I-2029 | 35 | 26.2 | | | 2012.846 | 1008.3 | 1006.2 | |
| I-2030 | 22 | 3.9 | | | 1936.815 | 970.3 | 1938.2 | |
| I-2031 | 11 | 4.7 | | | 1963.826 | 983.8 | 1964.2 | |
| I-2032 | 16 | 20.9 | | | 1991.857 | 997.9 | 1991.6 | |
| I-2033 | 30 | 9.5 | | | 2039.857 | 1021.9 | 1020 | |
| I-2034 | 18 | | | | 1963.826 | 983.8 | 981.9 | |
| I-2035 | 16 | 14.5 | | | 1963.826 | 1966.4 | 1964.5 | |
| I-2036 | 19 | 12.9 | | | 1963.826 | 983.8 | 981.5 | |
| I-2037 | 17 | 5.0 | | | 2039.857 | 2042.6 | 2039.7 | |
| I-2038 | 11 | 11.8 | | | 1979.821 | 991.8 | 989.7 | |
| I-2039 | 9 | 10.5 | | | 1979.821 | 991.9 | 989.7 | |
| I-2040 | 24 | 2.8 | | >20 | 2218.984 | 1111.5 | 1109.4 | |
| I-2041 | 16 | 2.3 | 1.9 | >20 | 2218.984 | 1111.4 | 1109.2 | |
| I-2042 | 9 | 10.0 | | | 1935.821 | 1937.9 | 1936 | |
| I-2043 | 8 | 2.8 | 1.0 | 15.2 | 1977.868 | 1980.1 | 1978.2 | |
| I-2044 | 9 | 2.6 | | >20 | 2011.852 | 2014.1 | 2012 | |
| I-2045 | 5 | 13.5 | | | 1951.816 | 1953.9 | 1951.9 | |
| I-2046 | 12 | 2.9 | | | 2190.979 | 2193.2 | 2191.2 | |
| I-2047 | 11 | 2.6 | 1.3 | | 2190.979 | 2193.3 | 2191 | |
| I-2048 | 91 | 28.8 | | | 2083.94 | 2085.5 | 2083.4 | |
| I-2049 | 5 | 4.5 | | | 1977.868 | 1980.2 | 1978.1 | |
| I-2050 | 9 | 4.9 | | | 1950.857 | 977.2 | 1951 | |
| I-2051 | 16 | 8.3 | | | 1964.873 | 1967 | 1965 | |
| I-2052 | 12 | 6.0 | | | 2025.868 | 2028.1 | 2025.8 | |
| I-2053 | 11 | 5.0 | | | 1949.837 | 1952 | 1949.9 | |
| I-2054 | 5 | 2.4 | | | 1977.868 | 1980.1 | 1978.1 | |
| I-2055 | 13 | 10.5 | | | 2019.915 | 2022.2 | 2020.2 | |
| I-2056 | 16 | 6.5 | | | 2033.931 | 1018.8 | 2034.3 | |
| I-2057 | 17 | 1.5 | 4.0 | >20 | 2053.915 | 1028.7 | 2054.1 | |
| I-2058 | 10 | 3.9 | | | 2039.899 | 2041.9 | 2040 | |
| I-2059 | 38 | 4.8 | | | 2012.888 | 2015.1 | 2013.1 | |
| I-2060 | 56 | 9.6 | | | 2026.904 | 2029.2 | 2027.2 | |
| I-2061 | 13 | 1.9 | 1.8 | | 2026.856 | 2029.1 | 2027.1 | |
| I-2062 | 41 | 3.0 | | | 2054.86 | 1029.4 | 1027.2 | |
| I-2063 | 56 | 3.5 | | | 2088.886 | 2091.1 | 2089.1 | |
| I-2064 | 16 | 11.8 | | | 2026.856 | 1015.2 | 2027.3 | |
| I-2065 | 144 | >>20 | | | 1991.002 | 1993.3 | 1991.1 | |
| I-2066 | 357 | | | | 1987.929 | 1990.2 | 1988.2 | |
| I-2067 | 180 | >>20 | | | 1978.929 | 1981.1 | 1979.3 | |
| I-2068 | 338 | >>20 | | | 2067.033 | 2069.3 | 2067.1 | |
| I-2069 | 256 | >>20 | | | 1957.919 | 1960.2 | 1957.9 | |
| I-2070 | 190 | >>20 | | | 1948.918 | 1951.1 | 1949.2 | |
| I-2071 | 592 | | | | 2027.964 | 2030.2 | 2018.1 | |
| I-2072 | 8256 | | | | 2027.964 | 2030.2 | 2028 | |
| I-2073 | 398 | | | | 1919.964 | 1922.2 | 1920.2 | |
| I-2074 | 789 | | | | 2026.969 | 2029.2 | 2026.9 | |
| I-2075 | 2327 | | | | 2026.969 | 2029.2 | 2027 | |
| I-2076 | 16 | 14.1 | | | 2024.916 | 2026.5 | 2024.5 | |
| I-2077 | 9 | 12.3 | 5.8 | | 2052.948 | 2054.4 | 2052.6 | |
| I-2078 | 5 | 11.7 | | | 2010.901 | 2012.4 | 2010.5 | |
| I-2079 | 8 | 15.1 | | | 2038.932 | 2040.5 | 2038.5 | |
| I-2080 | 45 | 28.8 | | | 2072.916 | | | |
| I-2081 | 13 | 11.6 | | | 2072.916 | | | |
| I-2082 | 5 | 7.7 | | | 2010.901 | 2012.5 | 2010.5 | |
| I-2083 | 5 | 3.7 | | | 2038.932 | 2040.5 | 2038.6 | |
| I-2084 | 5 | 9.2 | 9.7 | | 1996.885 | 1998.4 | 1996.5 | |
| I-2085 | 5 | 6.2 | | | 2024.916 | 2026.5 | 2024.6 | |
| I-2086 | 12 | 16.4 | | | 2058.901 | | | |
| I-2087 | 26 | 6.5 | | | 2021.906 | 2024.1 | 2022.1 | |
| I-2088 | 10 | 4.4 | | | 1991.884 | | | |
| I-2089 | 28 | 2.9 | | | 2053.915 | | | |
| I-2090 | 11 | 5.9 | | | 1934.862 | | | |
| I-2091 | 49 | 6.2 | | | 1996.893 | | | |
| I-2092 | 15 | 4.4 | | | 2025.868 | | | |
| I-2093 | 43 | 5.0 | | | 2087.899 | | | |
| I-2094 | 28 | 9.7 | | | 1968.847 | | | |
| I-2095 | 78 | 10.5 | | | 2030.878 | | | |
| I-2096 | 79 | >>20 | | | 1934.862 | | | |
| I-2097 | 23 | 22.3 | | | 1934.862 | | | |
| I-2098 | 260 | >>20 | | | 1996.893 | | | |
| I-2099 | 325 | | | | 1996.893 | 1999 | 1997.1 | |
| I-2100 | 86 | >>20 | | | 1934.862 | 1637.1 | 1935.2 | |
| I-2101 | 424 | | | | 1996.893 | 1000.1 | 1996.9 | |
| I-2102 | 273 | >>20 | | | 2077.111 | | | |
| I-2103 | 10000 | | | | 1994.001 | | | |
| I-2104 | 10000 | | | | 2008.017 | | | |
| I-2105 | 10000 | | | | 2035.028 | | | |
| I-2106 | 10000 | | | | 2044.028 | | | |
| I-2107 | 10000 | | | | 2060.989 | | | |
| I-2108 | 10000 | | | | 2045.023 | | | |
| I-2109 | 136 | >>20 | | | 2057.023 | | | |
| I-2110 | 88 | >>20 | | | 2048.059 | | | |
| I-2111 | 180 | >>20 | | | 2034.007 | | | |
| I-2112 | 501 | | | | 2021.012 | | | |
| I-2113 | 164 | | | | 2048.023 | | | |
| I-2114 | 32 | >>20 | | | 2022.853 | | | |
| I-2115 | 29 | >>20 | | | 2022.853 | | | |
| I-2116 | 19 | 33.4 | | | 2009.858 | | | |
| I-2117 | 19 | >>20 | | | 2009.858 | | | |
| I-2118 | 21 | >>20 | | | 1982.833 | | | |
| I-2119 | 29 | >>20 | | | 1982.833 | | | |
| I-2120 | 11 | 37.2 | | | 1969.838 | | | |
| I-2121 | 12 | >>20 | | | 1969.838 | | | |
| I-2122 | 12 | 34.4 | | | 1981.874 | | | |
| I-2123 | 12 | 26.9 | | | 1981.874 | | | |
| I-2124 | 29 | 38.9 | | | 1995.842 | | | |
| I-2125 | 26 | 41.2 | | | 1995.842 | | | |
| I-2126 | 68 | 29.5 | | | 2007.879 | | | |
| I-2127 | 36 | 40.8 | | | 2031.854 | | | |
| I-2128 | 29 | 27.0 | | | 2018.858 | | | |
| I-2129 | 105 | 44.0 | | | 2030.895 | | | |
| I-2130 | 4924 | | | | 2012.878 | 2014.3 | 2012.3 | |
| I-2131 | 3219 | | | | 2012.878 | 2014.4 | 2012.5 | |
| I-2132 | 10000 | | | | 1929.879 | 1931.4 | 1929.3 | |
| I-2133 | 8745 | | | | 1929.879 | 1931.5 | 1929.6 | |
| I-2134 | 10000 | | | | 1977.879 | 1979.5 | 1977.6 | |
| I-2135 | 3409 | | | | 1977.879 | 1979.5 | 1977.7 | |
| I-2136 | 5820 | | | | 1977.879 | 1979.4 | 1977.3 | |
| I-2137 | 1900 | | | | 1977.879 | 990.4 | 998.3 | |
| I-2138 | 1012 | | | | 1959.869 | 1961.9 | 1960 | |
| I-2139 | 970 | | | | 1959.869 | 1961.9 | 1959.9 | |
| I-2140 | 233 | | | | 1910.848 | 1912.9 | 1911 | |
| I-2141 | 310 | | | | 1910.848 | 1913 | 1911.2 | |
| I-2142 | 271 | >>20 | | | 1844.827 | 1846.8 | 1844.9 | |
| I-2143 | 217 | | | | 1844.827 | 1846.8 | 1845 | |
| I-2144 | 2023 | | | | 1907.863 | 1909.8 | 1907.9 | |
| I-2145 | 2068 | | | | 1907.863 | 955.5 | 1907.9 | |
| I-2146 | 248 | | | | 1924.864 | 1927 | 1925.2 | |
| I-2147 | 390 | | | | 1924.864 | 1927.1 | 1925 | |
| I-2148 | 10000 | | | | 1888.853 | 1890.8 | 1888.9 | |
| I-2149 | 7184 | | | | 1888.853 | 1890.9 | 1888.9 | |
| I-2150 | 647 | | | | 2146.903 | 2149.2 | 2147.2 | |
| I-2151 | 58 | 14.6 | | | 2146.903 | 2149.1 | 2147.1 | |
| I-2152 | 3083 | | | | 2013.822 | 2016.7 | 2014.9 | |
| I-2153 | 1484 | | | | 2007.866 | 2010.9 | 2008.8 | |
| I-2154 | 9549 | | | | 1985.881 | 1988.7 | 1986.8 | |
| I-2155 | 10000 | | | | 2075.853 | 2078.7 | 2077 | |
| I-2156 | 387 | >>20 | | | 1859.79 | 1861.7 | 1859.6 | |
| I-2157 | 141 | | | | 1873.806 | 1875.9 | 936.5 | |
| I-2158 | 213 | >>20 | | | 1887.821 | 1889.7 | 1887.8 | |
| I-2159 | 116 | >>20 | | | 1887.821 | 1889.9 | 1887.9 | |
| I-2160 | 1243 | | | | 1863.821 | | | A |
| I-2161 | 855 | >>20 | | | 1877.837 | 1879.7 | 1977.6 | A |
| I-2162 | 1163 | >>20 | | | 1891.852 | 1893.7 | 1891.8 | A |
| I-2163 | 3718 | | | | 2090.07 | 2007.1 | 2005 | |
| I-2164 | 1182 | | | | 2091.029 | 2093.3 | 2091.2 | |
| I-2165 | 441 | | | | 2020.053 | 2022.3 | 2020 | |
| I-2166 | 1586 | | | | 2063.059 | 2065.5 | 2063.3 | |
| I-2167 | 199 | >>20 | | | 2021.012 | 2023.2 | 2021 | |
| I-2168 | 212 | >>20 | | | 2064.018 | 2066.2 | 2064.1 | |
| I-2169 | 10000 | | | | 2064.018 | 2066.2 | 2064 | |
| I-2170 | 10000 | | | | 1987.987 | 1990.2 | 1988.1 | |
| I-2171 | 10000 | | | | 2002.002 | 2004.3 | 2002.6 | |
| I-2172 | 10000 | | | | 2037.007 | 2039.2 | 2037 | |
| I-2173 | 3072 | | | | 2064.018 | 2066.3 | 2064 | |
| I-2174 | 10000 | | | | 2064.018 | 2066.3 | 2064.1 | |
| I-2175 | 19 | | | | 1899.85 | 1901.8 | 1899.9 | |
| I-2176 | 87 | | | | 1933.811 | 1936.3 | 1934.4 | |
| I-2177 | 253 | | | | 1932.852 | 968.3 | 966.2 | |
| I-2178 | 42 | | | | 1917.841 | 1919.9 | 1917.9 | |
| I-2179 | 67 | | | | 1916.882 | 1918.9 | 1917.1 | |
| I-2180 | 2086 | | | | 1900.845 | 1902.9 | 1900.8 | |
| I-2181 | 2877 | | | | 1899.887 | 1901.9 | 1900 | |
| I-2182 | 5 | 11.1 | | | 1928.761 | 1931.7 | 1930.2 | |
| I-2183 | 5 | 5.0 | | | 1927.802 | 1930.8 | 1928.6 | |
| I-2184 | 358 | | | | 2108.835 | 1054.6 | 1052.5 | |
| I-2185 | 42 | 6.1 | | | 2108.835 | 1054.7 | 1052.4 | |
| I-2186 | 459 | | | | 2186.952 | 1095.2 | 2186.9 | |
| I-2187 | 28 | 10.8 | | | 2186.952 | 1095.2 | 2187 | |
| I-2188 | 11 | 7.6 | | | 2005.899 | 2008 | 2005.9 | |
| I-2189 | 83 | 33.4 | | | 2004.941 | 2092.5 | 2090.4 | |
| I-2190 | 28 | 33.4 | | | 1978.889 | 991.1 | 1979 | |
| I-2191 | 16 | 29.5 | | | 2033.931 | 2036.1 | 2034.1 | |
| I-2192 | 22 | 16.1 | | | 2047.946 | 1025.7 | 2048.3 | |
| I-2193 | 164 | 39.9 | | | 2032.972 | 2035.2 | 2033.2 | |
| I-2194 | 91 | 8.8 | | | 2004.941 | 2007.1 | 2005.1 | |
| I-2195 | 62 | 21.0 | | | 2006.92 | 2009.1 | 2007.1 | |
| I-2196 | 41 | 39.3 | | | 2061.962 | 1032.7 | 2062 | |
| I-2197 | 2448 | | | | 1887.832 | 1889.8 | 1887.9 | |
| I-2198 | 1465 | | | | 1887.832 | 1889.8 | 1888.1 | |
| I-2199 | 927 | | | | 1915.864 | 1917.8 | 1915.8 | |
| I-2200 | 8401 | | | | 1844.863 | 1846.8 | 1844.9 | |
| I-2201 | 10000 | | | | 1859.801 | 1861.7 | 1859.8 | |
| I-2202 | 11 | 5.0 | | | 1968.848 | 986 | 1968.9 | |
| I-2203 | 9 | 2.9 | | | 2037.879 | 2040.3 | 2038.4 | |
| I-2204 | 10 | 3.3 | | | 2037.879 | 2040.3 | 2038.4 | |
| I-2205 | 18 | 3.2 | | | 1996.852 | 1000.3 | 1997.4 | |
| I-2206 | 7 | 7.1 | | | 2009.874 | 1006.5 | 2010 | |
| I-2207 | 16 | 3.2 | | | 1966.869 | 1968.8 | 1966.9 | |
| I-2208 | 27 | 5.7 | | | 2059.871 | 2061.9 | 2060 | |
| I-2209 | 50 | 18.6 | | | 2018.845 | 1011.1 | 2018.8 | |
| I-2210 | 128 | 12.4 | | | 2046.849 | 1025.2 | 1023.3 | |
| I-2211 | 23 | 12.9 | | | 2059.871 | 1031.6 | 2060 | |
| I-2212 | 60 | 6.6 | | | 2016.865 | 1010 | 2017 | |
| I-2213 | 5252 | | | | 1889.848 | 1892.8 | 1890.9 | |
| I-2214 | 1857 | | | | 2005.987 | 2008.1 | 2006.3 | |
| I-2215 | 1542 | | | | 1969.987 | 1971.9 | 1970 | |
| I-2216 | 7 | 3.4 | | | 2003.834 | 2006.9 | 2005.2 | |
| I-2217 | 6 | 2.1 | | | 1989.818 | 997 | 995.1 | |
| I-2218 | 34 | 5.1 | | | 2024.838 | 1014.5 | 1012.8 | |
| I-2219 | 21 | 2.7 | | | 2065.865 | 1035 | 2067.1 | |
| I-2220 | 12 | 16.3 | | | 1969.873 | 1972.4 | 1969.8 | |
| I-2221 | 11 | 5.0 | | | 1955.857 | 1958.4 | 1955.8 | |
| I-2222 | 84 | 11.7 | | | 1990.877 | 1993.4 | 1992.3 | |
| I-2223 | 50 | 9.0 | | | 2031.904 | 1017.8 | 1015.7 | |
| I-2224 | 14 | 14.2 | | | 1969.873 | 1972.4 | 1969.7 | |
| I-2225 | 13 | 7.8 | | | 1955.857 | 979.8 | 977.5 | |
| I-2226 | 97 | 10.6 | | | 1990.877 | 997.3 | 995.2 | |
| I-2227 | 47 | 6.3 | | | 2031.904 | 1017.8 | 1015.7 | |
| I-2228 | 2142 | | | | 1722.717 | 1724.9 | 1722.9 | |
| I-2229 | 6 | | | | 2067.922 | 2070.1 | 2068.1 | |
| I-2230 | 8 | | | | 2067.922 | 2070.1 | 2068.3 | |
| I-2231 | 5187 | | | | 1850.812 | 1852.9 | 1851.2 | |
| I-2232 | 10000 | | | | 1850.812 | 1853.1 | 1851.3 | |
| I-2233 | 14 | >>20 | | | 2057.963 | 2060.3 | 2058 | |
| I-2234 | 22 | >>20 | | | 2043.947 | 2046.2 | 2044 | |
| I-2235 | 40 | >>20 | | | 2069.963 | 2072.4 | 2070 | |
| I-2236 | 23 | 15.3 | | | 2130.908 | 2133.1 | 2130.9 | |
| I-2237 | 5 | 24.3 | | | 2049.9 | 2052.1 | 2050.1 | |
| I-2238 | 11 | | | | 2147.974 | 2150.3 | 2148.2 | |
| I-2239 | 10 | 31.9 | | | 2071.942 | 2074.2 | 2071.9 | |
| I-2240 | 5 | >>20 | | | 2084.974 | 2087.3 | 2085.3 | |
| I-2241 | 18 | 17.7 | | | 2152.874 | 2155.2 | 2153 | |
| I-2242 | 13 | 21.4 | | | 2138.858 | 2141 | 2139.1 | |
| I-2243 | 15 | 25.2 | | | 2071.979 | 2074.2 | 2072 | |
| I-2244 | 16 | 27.4 | | | 2057.963 | 2060.2 | 2058 | |
| I-2245 | 26 | >>20 | | | 2083.979 | 2086.2 | 2084 | |
| I-2246 | 27 | 12.9 | | | 2144.924 | 2147 | 2145 | |
| I-2247 | 8 | 5.9 | | | 2063.916 | 2066 | 2064.1 | |
| I-2248 | 8 | 21.5 | | | 2161.989 | 2164.2 | 2162.1 | |
| I-2249 | 14 | 42.2 | | | 2085.958 | 2088.2 | 2086.1 | |
| I-2250 | 12 | 36.2 | | | 2098.99 | 2101.2 | 2099.1 | |
| I-2251 | 15 | 6.4 | | | 2166.889 | 2168.9 | 2167.1 | |
| I-2252 | 31 | 17.5 | | | 2152.874 | 2154.9 | 2153 | |
| I-2253 | 189 | | | | 1996.828 | 1000.6 | 998.3 | |
| I-2254 | 5792 | | | | 2011.943 | 2013.8 | 2011.9 | |
| I-2255 | 7647 | | | | 1984.002 | 1985.9 | 1983.9 | |
| I-2256 | 8412 | | | | 2073.974 | 2075.9 | 2073.9 | |
| I-2257 | 15 | 2.9 | | | 1934.862 | | | |
| I-2258 | 272 | | | | 1950.857 | | | |
| I-2259 | 30 | 4.3 | | | 1968.847 | | | |
| I-2260 | 21 | 3.1 | | | 1952.853 | | | |
| I-2261 | 34 | 6.4 | | | 1946.896 | | | |
| I-2262 | 18 | 3.7 | | | 1952.853 | | | |
| I-2263 | 47 | 4.3 | | | 1946.896 | | | |
| I-2264 | 45 | 25.5 | | | 2084.888 | 2087.1 | 2085 | |
| I-2265 | 67 | >>20 | | | 2088.919 | 2091.2 | 2089 | A |
| I-2266 | 266 | | | | 2048.833 | 2051.1 | 2048.9 | |
| I-2267 | 499 | | | | 2052.865 | 2055.1 | 2052.9 | A |
| I-2268 | 13 | 3.1 | | 4.3 | 2333.054 | | | |
| I-2269 | 20 | 2.3 | | 3.9 | 2475.128 | | | |
| I-2270 | 40 | | | | 2617.202 | | | |
| I-2271 | 13 | 2.3 | | 9.0 | 2204.995 | | | |
| I-2272 | 43 | 3.0 | | | 2361.085 | | | |
| I-2273 | 107 | 6.2 | | | 2517.175 | | | |
| I-2274 | 10 | 2.0 | | 6.2 | 2090.952 | | | |
| I-2275 | 117 | 3.5 | | | 2317.12 | | | |
| I-2276 | 157.5 | 4.0 | 3.9 | >20 | 2317.12 | | | |
| I-2277 | 114 | 2.3 | | 9.0 | 2275.073 | | | |
| I-2278 | 119.5 | 2.8 | | >20 | 2275.073 | | | |
| I-2279 | 7233 | >>20 | | | 2543.288 | | | |
| I-2280 | 10000 | >>20 | >>20 | >20 | 2543.288 | | | |
| I-2281 | 138 | 4.3 | | | 2417.148 | | | |
| I-2282 | 155.5 | 3.7 | | | 2417.148 | | | |
| I-2283 | 2862 | 4.5 | | | 2601.269 | | | |
| I-2284 | 2683 | 12.9 | | | 2601.269 | | | |
| I-2285 | 94 | | | | 2017.738 | 1011.1 | 1008.5 | |
| I-2286 | 88 | | | | 2040.777 | 1022.9 | 2043.1 | |
| I-2287 | 7 | 7.1 | | | 2031.813 | 2034.8 | 2032.5 | |
| I-2288 | 5 | 6.7 | | | 2017.797 | 2020.8 | 2018.8 | |
| I-2289 | 8 | 9.0 | | | 2052.817 | 2055.8 | 2053.8 | |
| I-2290 | 14 | 7.9 | | | 2093.844 | 2096.8 | 2094.9 | |
| I-2291 | 5 | 4.9 | | | 1963.943 | 1965.8 | 1963.9 | |
| I-2292 | 9 | 2.7 | | | 1949.927 | 1951.9 | 1950 | |
| I-2293 | 15 | 5.7 | | | 1984.947 | 994 | 992.1 | |
| I-2294 | 16 | 4.6 | | | 2025.974 | 2027.9 | 2025.9 | |
| I-2295 | 5 | 3.3 | | | 1975.943 | 1977.9 | 1976 | |
| I-2296 | 6 | 5.1 | | | 1961.927 | 1963.9 | 1961.9 | |
| I-2297 | 68 | 5.5 | | | 1996.947 | 1998.9 | 1997 | |
| I-2298 | 8 | 4.6 | | | 2037.974 | 2039.9 | 2038 | |
| I-2299 | 7 | 5.2 | | | 2166.999 | 2169.1 | 2167.2 | |
| I-2300 | 6 | 1.2 | | >20 | 2166.999 | 2169.1 | 2167.3 | |
| I-2301 | 5 | >>20 | | | 2076.973 | 2079.2 | 2077.3 | |
| I-2302 | 5 | 2.5 | | | 2005.899 | 2007.9 | 2005.8 | |
| I-2303 | 5 | 1.2 | | | 2005.899 | 2008 | 2006 | |
| I-2304 | 511 | | | | 2020.909 | | | |
| I-2305 | 65 | >>20 | | | 2020.909 | | | |
| I-2306 | 45 | >>20 | | | 1897.769 | 1899.3 | 1897.5 | |
| I-2307 | 16 | 8.9 | | | 2013.822 | 2016.8 | 2015.1 | |
| I-2308 | 8 | 6.0 | | | 1999.807 | 1002 | 1000.2 | |
| I-2309 | 17 | 13.7 | | | 1985.927 | 1988 | 1986.1 | |
| I-2310 | 18 | 8.9 | | | 1971.912 | 1973.9 | 1971.5 | |
| I-2311 | 5 | 5.2 | | | 1999.943 | 2001.9 | 2000.1 | |
| I-2312 | 5 | 5.2 | | | 1985.927 | 1987.9 | 1985.9 | |
| I-2313 | 22 | 24.3 | | | 1999.943 | 2001.9 | 2000.1 | |
| I-2314 | 48 | >>20 | | | 1985.927 | 1988 | 1986.1 | |
| I-2315 | 5 | 1.2 | 1.5 | >20 | 2123.957 | 1063.3 | 1061.3 | |
| I-2316 | 11 | 1.6 | | >20 | 2204.976 | 2206.3 | 2204.4 | |
| I-2317 | 12 | 18.8 | | | 2205.051 | 2206.6 | 2204.6 | |
| I-2318 | 5 | 1.8 | | >20 | 2136.952 | 2138.3 | 2136.4 | |
| I-2319 | 5 | 1.0 | 1.7 | >20 | 2164.983 | 1083.8 | 1081.8 | |
| I-2320 | 29 | 2.7 | | >20 | 2155.962 | 1079.3 | 1076.9 | |
| I-2321 | 5 | 1.7 | | | 2214.966 | 1108.8 | 1106.8 | |
| I-2322 | 7 | 1.8 | | >20 | 2169.998 | 2171.3 | 2169.4 | |
| I-2323 | 6 | 17.7 | | | 2208.062 | 2209.5 | 2207.5 | |
| I-2324 | 83 | 14.8 | | | 1978.862 | | | |
| I-2325 | 1353 | | | | 2046.924 | | | |
| I-2326 | 185 | | | | 2017.838 | | | |
| I-2327 | 376 | | | | 2040.877 | | | |
| I-2328 | 170 | >>20 | | | 2166.929 | 2169.1 | 2167.1 | A |
| I-2329 | 3795 | | | | 2039.891 | 2042 | 2040.1 | |
| I-2330 | 3856 | | | | 2025.875 | 2027.9 | 2025.8 | |
| I-2331 | 10000 | | | | 1915.864 | 1917.4 | 1915.3 | |
| I-2332 | 10000 | | | | 1915.864 | 1917.3 | 1915.3 | |
| I-2333 | 10000 | | | | 1900.864 | 1902.3 | 1900.3 | |
| I-2334 | 656 | | | | 1831.831 | 917.3 | 1831.3 | |
| I-2335 | 7 | 37.0 | | | 1991.859 | 1993.4 | 1991.3 | |
| I-2336 | 19 | 14.4 | | | 1978.863 | 1980.4 | 1978.3 | |
| I-2337 | 311 | | | | 1897.853 | 1899.8 | 1897.7 | |
| I-2338 | 63 | >>20 | | | 1915.864 | 1917.5 | 1915.5 | |
| I-2339 | 2816 | | | | 1915.864 | 1917.4 | 1915.5 | |
| I-2340 | 5 | >>20 | | | 2110.064 | 2112 | 2109.9 | |
| I-2341 | 42 | >>20 | | | 2145.062 | 2146.8 | 2144.8 | |
| I-2342 | 2725 | | | | 2109.08 | 2110.9 | 2108.8 | |
| I-2343 | 10000 | | | | 2109.08 | 2110.8 | 2108.8 | |
| I-2344 | 586 | | | | 2020.053 | 2021.8 | 2019.6 | |
| I-2345 | 1267 | | | | 2020.053 | 2021.7 | 2019.7 | |
| I-2346 | 422 | | | | 2047.101 | 2048.9 | 2046.9 | |
| I-2347 | 5632 | | | | 2047.101 | 1025 | 2047.4 | |
| I-2348 | 179 | | | | 2056.064 | 2057.7 | 2055.7 | |
| I-2349 | 1155 | | | | 2056.064 | 2057.6 | 2055.7 | |
| I-2350 | 63 | >>20 | | | 2047.101 | 2048.8 | 2046.8 | |
| I-2351 | 187 | | | | 2056.064 | 2057.9 | 2056 | |
| I-2352 | 211 | | | | 1990.043 | 1991.8 | 1989.7 | |
| I-2353 | 2757 | | | | 2034.069 | 2035.8 | 2033.8 | |
| I-2354 | 100 | >>20 | | | 1977.97 | 1979.4 | 1977.3 | |
| I-2355 | 417 | | | | 1977.97 | 1979.3 | 1977.3 | |
| I-2356 | 5274 | | | | 1943.884 | 1945.7 | 1943.8 | |
| I-2357 | 6989 | >>20 | | | 1943.884 | 1945.7 | 1943.8 | |
| I-2358 | 463 | 52.2 | | | 1943.884 | 1945.7 | 1943.8 | |
| I-2359 | 424 | >>20 | | | 1929.868 | 1931.7 | 1929.8 | |
| I-2360 | 2443 | >>20 | | | 1929.868 | 1931.7 | 1929.8 | |
| I-2361 | 349 | 36.7 | | | 1929.868 | 1931.7 | 1929.8 | |
| I-2362 | 66 | | | | 1955.822 | 1957.7 | 1955.9 | |
| I-2363 | 44 | | | | 1997.869 | 1999.7 | 1998.6 | |
| I-2364 | 116 | | | | 1997.869 | 1999.7 | 1998.2 | |
| I-2365 | 3442 | | | | 1957.899 | 980.5 | 1958.4 | |
| I-2366 | 1737 | | | | 1943.884 | 973.4 | 1943.7 | |
| I-2367 | 7 | 6.2 | | | 2072.932 | 1038 | 1036 | |
| I-2368 | 5 | 5.0 | | | 2100.963 | 2102.8 | 2100.9 | |
| I-2369 | 14 | 9.5 | | | 2086.947 | 1045 | 1043 | |
| I-2370 | 12 | | | | 2031.905 | 2033.4 | 2031.5 | |
| I-2371 | 5 | 5.0 | | | 2031.905 | 2033.7 | 2031.7 | |
| I-2372 | 5 | 2.6 | 2.1 | 7.0 | 2059.937 | 2061.6 | 2059.8 | |
| I-2373 | 5 | 4.0 | | | 2017.89 | 2019.6 | 2017.7 | |
| I-2374 | 7 | 3.4 | 2.6 | | 2045.921 | 1024.5 | 1022.5 | |
| I-2375 | 5 | 13.5 | | | 2009.942 | 2011.7 | 2009.6 | |
| I-2376 | 352 | | | | 2011.871 | 2013.4 | 2011.6 | A |
| I-2377 | 978 | | | | 2108.924 | 2110.5 | | A |
| I-2378 | 31 | 4.8 | 1.0 | | 2238.036 | 1120.4 | 1118.5 | |
| I-2379 | 245.5 | 5.0 | | | 2266.067 | 1134.5 | 1132.6 | |
| I-2380 | 329.5 | 2.7 | 1.5 | >20 | 2280.083 | 1141.5 | 1139.5 | |
| I-2381 | 1471 | 2.1 | 1.1 | >20 | 2314.067 | 1158.5 | 1156.5 | |
| I-2382 | 37 | 4.0 | 7.6 | | 2295.057 | 2296.8 | 2294.9 | |
| I-2383 | 77 | 4.2 | 0.8 | | 2268.046 | 1135.4 | 1133.5 | |
| I-2384 | 5 | 4.6 | 1.1 | | 2254.031 | 2255.7 | 2253.9 | |
| I-2385 | 563.5 | 4.0 | 1.8 | | 2280.083 | 1141.5 | 1139.4 | |
| I-2386 | 75.5 | 2.8 | 0.7 | | 2252.052 | 1127.6 | 1125.6 | |
| I-2387 | 120 | 7.1 | 1.5 | | 2278.067 | 1140.6 | 1138.4 | |
| I-2388 | 133 | 5.7 | 1.2 | | 2264.052 | 1133.5 | 1131.4 | |
| I-2389 | 72 | | | | 2250.036 | 1126.4 | 1124.4 | |
| I-2390 | 22 | 9.1 | 1.4 | | 2281.042 | 2282.8 | 2280.9 | |
| I-2391 | 110 | 8.2 | 0.7 | | 2280.083 | 1141.5 | 1139.5 | |
| I-2392 | 461.5 | 8.6 | | | 2330.062 | 2331.9 | 2330.1 | |
| I-2393 | 61 | >>20 | 5.6 | | 2323.1 | 2325.1 | 2323.1 | |
| I-2394 | 534.5 | 2.2 | 0.7 | >20 | 2353.078 | 1178.1 | 1176.1 | |
| I-2395 | 28 | 5.5 | 1.2 | | 2238.036 | 2239.7 | 2237.8 | |
| I-2396 | 187 | 8.3 | 1.5 | | 2280.083 | 1141.5 | 1139.5 | |
| I-2397 | 53 | 7.8 | | | 2268.046 | 1135.5 | 1133.5 | |
| I-2398 | 28 | 3.6 | 1.3 | | 2295.057 | 2296.8 | 2294.9 | |
| I-2399 | 17 | 2.8 | 2.2 | | 2195.989 | 2197.7 | 2195.8 | |
| I-2400 | 142.5 | 2.5 | 0.8 | | 2272.02 | 1137.5 | 1135.5 | |
| I-2401 | 37 | 4.4 | 0.8 | | 2224.02 | 1113.5 | 1111.5 | |
| I-2402 | 16 | >>20 | | | 1949.812 | 1951.4 | 1949.2 | |
| I-2403 | 12 | >>20 | | | 1972.851 | 987.9 | 985.8 | |
| I-2404 | 781 | | | | 2025.887 | 2027.6 | 2025.6 | A |
| I-2405 | 568 | | | | 2122.94 | 2124.8 | 2123 | A |
| I-2406 | 242 | | | | 2180.945 | 2182.7 | 2180.8 | A |
| I-2407 | 138 | >>20 | | | 1957.922 | 1959.6 | 1957.4 | |
| I-2408 | 10000 | | | | 1914.905 | 1916.3 | 1914.3 | |
| I-2409 | 397 | 50.0 | | | 1902.868 | 1904.3 | 1902.3 | |
| I-2410 | 304 | >>20 | | | 1938.88 | 1940.3 | 1938.3 | |
| I-2411 | 140 | | | | 1858.842 | 1860.7 | 1858.9 | |
| I-2412 | 267 | | | | 1934.874 | 1936.7 | 1934.8 | |
| I-2413 | 133 | | >>20 | | 1943.906 | 1945.6 | 1943.3 | |
| I-2414 | 7 | >>20 | | | 1924.864 | 1926.8 | 1924.8 | |
| I-2415 | 147 | >>20 | | | 1901.848 | 1903.3 | 1901.2 | |
| I-2416 | 133 | >>20 | >>20 | | 1888.853 | 1890.1 | 1888.2 | |
| I-2417 | 158 | 21.6 | | | 1927.814 | 1930 | 1927.8 | |
| I-2418 | 10000 | | | | 1947.996 | 1949.7 | 1947.3 | |
| I-2419 | 203 | >>20 | | | 1978.006 | 1979.6 | 1977.3 | |
| I-2420 | 2125 | | | | 1991.002 | 1992.6 | 1990.3 | |
| I-2421 | 145 | >>20 | | | 2005.017 | 2006.7 | 2004.6 | |
| I-2422 | 3360 | | | | 1963.991 | 1965.6 | 1963.3 | |
| I-2423 | 10000 | | | | 1978.006 | 1979.6 | 1977.4 | |
| I-2424 | 10000 | | | | 1962.011 | 1963.6 | 1961.4 | |
| I-2425 | 10000 | | | | 2061.043 | 2062.4 | 2060.4 | |
| I-2426 | 46 | >>20 | >>20 | | 2103.09 | 2104.7 | 2102.6 | |
| I-2427 | 757 | | | | 2061.08 | 2062.5 | 2060.6 | |
| I-2428 | 401 | | | | 2103.127 | 2104.5 | 2102.5 | |
| I-2429 | 10 | >>20 | | | 2048.085 | 2049.4 | 2047.5 | |
| I-2430 | 32 | 4.5 | | | 2095.962 | 2097.9 | 2095.9 | |
| I-2431 | 33 | 7.0 | | | 2095.962 | 1049.5 | 1047.2 | |
| I-2432 | 60 | | | | 2235.036 | 2236.4 | 2234.5 | |
| I-2433 | 10000 | | | | 1959.848 | 981.6. | 1960.1 | |
| I-2434 | 10000 | | | | 1978.965 | 990.8. | 1978.2 | |
| I-2435 | 10000 | | | | 1935.996 | 1937.1 | 1935.1 | |
| I-2436 | 10000 | | | | 1894.933 | 948.8. | 946.7. | |
| I-2437 | 10000 | | | | 1937.939 | 1939.2 | 1937.2 | |
| I-2438 | 1762 | | | | 1905.912 | 1907.3 | 1905.1 | |
| I-2439 | 2762 | | | | 1932.923 | 1934.4 | 1932.2 | |
| I-2440 | 360 | | | | 1945.919 | 1947.4 | 1945.2 | |
| I-2441 | 10000 | | | | 1924.955 | 1926.4 | 1924.2 | |
| I-2442 | 282 | | | | 1930.944 | 1932.3 | 1930.2 | |
| I-2443 | 6674 | | | | 1908.948 | 855.8. | 853.5. | |
| I-2444 | 121 | | | | 2235.036 | 1118.8 | 1116.7 | |
| I-2445 | 5 | 17.1 | 9.5 | | 2022.901 | 2024.1 | 2022.2 | |
| I-2446 | 93 | >>20 | | | 2053.021 | 2054.3 | 2052.4 | |
| I-2447 | 3249 | | | | 1950.959 | 1958.2 | 1956.3 | |
| I-2448 | 84 | >>20 | | | 2020.053 | 2027.5 | 2025.4 | |
| I-2449 | 10000 | | | | 1950.995 | 1958.6 | 1956.3 | |
| I-2450 | 63 | >>20 | | | 2020.09 | 2027.4 | 2025.4 | |
| I-2451 | 4861 | | | | 1987.958 | 1989.3 | 1987.4 | A |
| I-2452 | 7201 | | | | 2057.052 | 2058.6 | 2054.4 | A |
| I-2453 | 10000 | | | | 2024.085 | 2031.6 | 2029.4 | A |
| I-2454 | 10000 | | | | 1955.027 | 1962.5 | 1960.2 | A |
| I-2455 | 10000 | | | | 2024.121 | 2031.4 | 2029.4 | A |
| I-2456 | 49 | 8.1 | | | 2067.93 | 2068.9 | 2067.1 | |
| I-2457 | 52 | 20.8 | | | 2067.93 | 2090.8 | 2067 | |
| I-2458 | 33 | 5.9 | | | 2067.93 | 2068.9 | 2067 | |
| I-2459 | 56 | 15.1 | | | 2067.93 | 2090.8 | 2067.1 | |
| I-2460 | 82 | 12.8 | | | 2047.958 | 2048.9 | 2047 | |
| I-2461 | 75 | 25.4 | | | 2047.958 | 2070.8 | 2047 | |
| I-2462 | 40 | 4.7 | | | 2061.974 | 2063.1 | 2061.2 | |
| I-2463 | 47 | 6.5 | | | 2061.974 | 1032.1 | 2061.1 | |
| I-2464 | 79 | 16.5 | | | 2061.974 | 2063.1 | 2061.2 | |
| I-2465 | 164 | >>20 | | | 2061.974 | 2084.8 | 2061 | |
| I-2466 | 15 | | | | 2152.983 | 2153.9 | 2152.1 | |
| I-2467 | 10 | 2.5 | 1.4 | | 2125.95 | 2127 | 2125 | |
| I-2468 | 38 | 6.2 | 6.4 | >20 | 2138.968 | 2140 | 2138.1 | |
| I-2469 | 7 | 2.3 | 0.6 | 5.6 | 2076.937 | 2078 | 2076.1 | |
| I-2470 | 24 | | | | 2152.983 | 2153.9 | 2152 | |
| I-2471 | 14 | 2.1 | | | 2125.95 | 2126.9 | 2125.1 | |
| I-2472 | 13 | 1.8 | | | 2124.952 | 2125.8 | 2123.9 | |
| I-2473 | 7 | | | | 2076.937 | 2077.9 | 2076 | |
| I-2474 | 19 | | | | 2165.982 | 2167 | 2165.1 | |
| I-2475 | 28 | | | | 2164.983 | 2187.8 | 2164 | |
| I-2476 | 7 | | | | 2116.968 | 2118 | 2116.1 | |
| I-2477 | 165 | >>20 | | | 1915.9 | 1917.3 | 1915.1 | |
| I-2478 | 310 | >>20 | | | 1950.868 | 976.6. | 974.7. | |
| I-2479 | 627 | | | | 1900.889 | 951.7. | 950.18 | |
| I-2480 | 475 | | | | 1886.874 | 944.6. | 942.6. | |
| I-2481 | 754 | | | | 1973.884 | 988.2. | 986.19 | |
| I-2482 | 5 | | | | 2036.916 | 2038.1 | 2036.1 | |
| I-2483 | 5 | | | | 2078.963 | 2080.1 | 2078.2 | |
| I-2484 | 6291 | >>20 | | | 2104.941 | 2105.9 | 2104.2 | |
| I-2485 | 4754 | >>20 | | | 2104.941 | 2127.8 | 2104.1 | |
| I-2486 | 34 | 3.7 | | | 2087.876 | 2089.3 | 2087.6 | |
| I-2487 | 36 | 4.2 | | | 2087.876 | 1045.3 | 1043.4 | |
| I-2488 | 26 | 3.6 | | | 2071.905 | 2072.9 | 2071.2 | |
| I-2489 | 29 | 7.4 | | | 2071.905 | 1037.1 | 1035.1 | |
| I-2490 | 39 | 6.4 | | | 2087.876 | 2089.4 | 2087.5 | |
| I-2491 | 49 | 6.1 | | | 2087.876 | 1045.3 | 1043.4 | |
| I-2492 | 174 | >>20 | | | 1913.848 | 1917.3 | 1915.1 | |
| I-2493 | 267 | >>20 | | | 1941.879 | 976.6. | 974.7. | |
| I-2494 | 102 | >>20 | | | 1929.879 | 951.7. | 950.19 | |
| I-2495 | 95 | >>20 | | | 1915.864 | 944.6. | 942.6. | |
| I-2496 | 106 | >>20 | | | 1929.879 | 988.2. | 986.19 | |
| I-2497 | 105 | >>20 | | | 1915.864 | 1916.9 | 1914.9 | |
| I-2498 | 449 | | | | 2033.996 | 2035.1 | 2033 | |
| I-2499 | 10000 | | | | 2046.032 | 2047.1 | 2045.1 | |
| I-2500 | 10000 | | | | 2074.064 | 2075.2 | 2073.1 | |
| I-2501 | 154 | >>20 | | | 2089.038 | 2090.2 | 2088.1 | |
| I-2502 | 214 | >>20 | | | 2075.023 | 2076.1 | 2074 | |
| I-2503 | 1306 | | | | 2033.996 | 2035.1 | 2033.1 | |
| I-2504 | 210 | >>20 | | | 2061.007 | 2062 | 2060.1 | |
| I-2505 | 687 | | | | 2053.915 | 2054.8 | 2052.9 | |
| I-2506 | 332 | | | | 2053.915 | 1028 | 2052.9 | |
| I-2507 | 168 | | | | 2053.915 | 1028.1 | 2052.9 | |
| I-2508 | 152 | 27.3 | | | 2067.93 | 2068.9 | 2067 | |
| I-2509 | 17 | | | | 2067.93 | 2069 | 2067 | |
| I-2510 | 24 | 7.8 | | | 2067.93 | 1035.1 | 2066.9 | |
| I-2511 | 566 | | | | 2081.946 | 2082.9 | 2081 | |
| I-2512 | 245 | | | | 2081.946 | 2183.1 | 2181.2 | |
| I-2513 | 66 | >>20 | | | 2081.946 | 2082.9 | 2081 | |
| I-2514 | 74 | | | | 2081.946 | 2082.8 | 2080.9 | |
| I-2515 | 1608 | >>20 | | | 2081.946 | 1042.1 | 2080.9 | |
| I-2516 | 209 | | | | 2081.946 | 1042.1 | 2081 | |
| I-2517 | 61 | 30.0 | | | 2081.946 | 1042.1 | 2081 | |
| I-2518 | 212 | >>20 | | | 2053.915 | 2054.8 | 2052.9 | |
| I-2519 | 94 | | | | 2067.93 | 1035.1 | 2067.2 | |
| I-2520 | 27 | 5.9 | 5.7 | | 2140.994 | 1071.7 | 2140.3 | |
| I-2521 | 38 | 5.6 | 3.0 | | 2099.968 | 1051.2 | 2100 | |
| I-2522 | 219 | >>20 | | | 2103.054 | 2104.2 | 2102.1 | |
| I-2523 | 1732 | >>20 | | | 2165.07 | 2166.2 | 2164.2 | |
| I-2524 | 609 | | | | 2139.021 | 2140.2 | 2138.1 | |
| I-2525 | 4287 | | | | 2157.026 | 2158 | 2156.1 | |
| I-2526 | 224 | >>20 | | | 2279.159 | 2280.2 | 2278.3 | |
| I-2527 | 263 | >>20 | | | 2188.107 | 2189.3 | 2187.2 | |
| I-2528 | 610 | | | | 2160.112 | 2161.3 | 2159.2 | |
| I-2529 | 153 | 33.2 | | | 2055.93 | | | A |
| I-2530 | 102 | | | | 2041.915 | | | A |
| I-2531 | 179 | | | | 2043.93 | | | A |
| I-2532 | 14 | 4.5 | | >20 | 2081.946 | | | |
| I-2533 | 28 | 6.2 | | >20 | 2081.946 | 2083.1 | 2081.2 | |
| I-2534 | 21 | 4.4 | | | 2115.93 | 2117.2 | 2115.3 | |
| I-2535 | 11 | 3.7 | | | 2124.952 | | | |
| I-2536 | 13 | 4.5 | | | 2124.952 | 2126.1 | 2124.2 | |
| I-2537 | 11 | 2.9 | | | 2166.962 | 2168.2 | 2166.3 | |
| I-2538 | 14 | 3.5 | | | 2166.962 | 2168.1 | 2166.2 | |
| I-2539 | 11 | 3.6 | | | 2138.968 | 2140.2 | 2138.2 | |
| I-2540 | 14 | 3.5 | | | 2138.968 | | | |
| I-2541 | 21 | 4.7 | | >20 | 2123.957 | 2125.1 | 2123.2 | |
| I-2542 | 23 | 4.0 | | >20 | 2123.957 | 2125.1 | 2123.3 | |
| I-2543 | 37 | 7.3 | | | 2157.941 | 2159.1 | 2157.2 | |
| I-2544 | 11 | 4.1 | | | 2166.962 | 2168.1 | 2166.2 | |
| I-2545 | 16 | 4.0 | | | 2166.962 | 2168.1 | 2166.3 | |
| I-2546 | 13 | 3.5 | | | 2208.973 | | | |
| I-2547 | 13 | 3.5 | | | 2208.973 | 2210.1 | 2208.3 | |
| I-2548 | 11 | 3.6 | | | 2180.978 | 2182.1 | 2180.2 | |
| I-2549 | 13 | 2.5 | | | 2180.978 | 2182.2 | 2180.3 | |
| I-2550 | 1721 | | | | 2035.871 | 2036.7 | 2034.8 | |
| I-2551 | 8 | 9.8 | | | 2033.931 | 2035.2 | 2033.3 | |
| I-2552 | 12 | 10.4 | | | 2033.931 | 2035.2 | 2033.4 | |
| I-2553 | 10 | 8.2 | | | 2075.941 | 2077.2 | 2075.4 | |
| I-2554 | 8 | 3.3 | | | 2121.983 | 2123.6 | 2121.7 | |
| I-2555 | 11 | 22.1 | | | 2157.036 | 2458.7 | 2156.5 | |
| I-2556 | 6 | 7.0 | | | 2116.968 | 2118.3 | 2116.5 | |
| I-2557 | 29 | 19.6 | | | 2141.949 | 2143.2 | 2141.4 | |
| I-2558 | 7 | 14.7 | | | 2047.91 | 2049.2 | 2047.3 | |
| I-2559 | 8 | 3.8 | | | 2093.952 | 2095.5 | 2093.6 | |
| I-2560 | 9 | 19.3 | | | 2129.004 | 2130.8 | 2128.6 | |
| I-2561 | 5 | 7.2 | | | 2088.937 | 2090.2 | 2088.5 | |
| I-2562 | 10000 | | | | 2120.821 | 2123.3 | 2121.6 | |
| I-2563 | 48 | 17 | | | 2027.899 | 2029.2 | 2027.3 | |
| I-2564 | 259 | | | | 2055.93 | 2057.2 | 2055.2 | |
| I-2565 | 90 | 21 | | | 2055.93 | 2057.2 | 2055.3 | |
| I-2566 | 181 | | | | 2124.952 | 2126.3 | 2124.4 | |
| I-2567 | 522 | | | | 2152.983 | 2154.3 | 2152.4 | |
| I-2568 | 4770 | | | | 2183.953 | 2185.2 | 2183.1 | |
| I-2569 | 1948 | | | | 2241.958 | 1122.3 | 1120.3 | |
| I-2570 | 1278 | | | | 2031.93 | 2033.2 | 2031.3 | A |
| I-2571 | 42 | 24 | | | 2059.962 | 2061.2 | 2059.4 | A |
| I-2572 | 67 | >>20 | | | 2128.983 | 2130.3 | 2128.4 | A |
| I-2573 | 234 | | | | 2157.014 | 1079.8 | 1077.9 | A |
| I-2574 | 2960 | | | | 2130.999 | 2133.3 | 2131.2 | A |
| I-2575 | 8156 | | | | 2282.026 | 1133.3 | 1131.4 | |
| I-2576 | 6 | 16.1 | | >20 | 2009.858 | 2011.2 | 2009.4 | |
| I-2577 | 7 | 2.7 | | 9.5 | 2009.858 | 2011.4 | 2009.5 | |
| I-2578 | 5 | 5.5 | 4.1 | 7.7 | 2076.937 | 1639.7 | 1637.8 | |
| I-2579 | 41 | 4.7 | | 15.3 | 2138.968 | 1070.7 | 1068.8 | |
| I-2580 | 993 | | | | 2053.915 | 2055.5 | 2053.6 | |
| I-2581 | 205 | >>20 | | | 2067.93 | 2069.6 | 2067.8 | |
| I-2582 | 263 | >>20 | | | 2067.93 | 2069.6 | 2067.6 | |
| I-2583 | 224 | >>20 | | | 2081.946 | 2083.6 | 2081.9 | |
| I-2584 | 1840 | | | | 2053.915 | 2055.7 | 2053.9 | |
| I-2585 | 452 | | | | 2067.93 | 2069.8 | 2068.1 | |
| I-2586 | 345 | | | | 2067.93 | 2069.8 | 2068 | |
| I-2587 | 273 | | | | 2081.946 | 2083.7 | 2081.9 | |
| I-2588 | 26 | | | | 2005.899 | 2007.6 | 2005.8 | |
| I-2589 | 25 | 15.2 | | | 2005.899 | 2007.6 | 2005.8 | |
| I-2590 | 25 | 18.9 | | | 2019.915 | 2021.6 | 2019.8 | |
| I-2591 | 602 | | | | 1991.884 | 1993.6 | 1991.8 | |
| I-2592 | 37 | >>20 | | | 2005.899 | 2007.6 | 2005.5 | |
| I-2593 | 27 | 29.4 | | | 2005.899 | 2007.6 | 2006 | |
| I-2594 | 470 | >>20 | | | 2042.877 | 2044.5 | 2042.8 | B |
| I-2595 | 356 | >>20 | | | 2042.877 | 2044.5 | 2042.9 | B |
| I-2596 | 10000 | | | | 2069.91 | 2071.6 | 2069.9 | B |
| I-2597 | 726 | | | | 2069.91 | 2069.6 | 2067.7 | B |
| I-2598 | 1346 | | | | 1887.821 | 1889.4 | 1887.8 | |
| I-2599 | 234 | >>20 | | | 1873.806 | 1875.5 | 1873.9 | |
| I-2600 | 923 | | | | 1891.852 | 1893.5 | 1891.6 | A |
| I-2601 | 592 | | | | 1877.837 | 1879.5 | 1877.7 | A |
| I-2602 | 8217 | | | | 1963.852 | 1965.5 | 1963.7 | |
| I-2603 | 767 | | | | 1949.837 | 1951.5 | 1949.9 | |
| I-2604 | 1119 | | | | 1967.884 | 1969.6 | 1967.8 | A |
| I-2605 | 608 | | | | 1953.868 | 1955.5 | 1953.8 | A |
| I-2606 | 734 | | >>20 | | 1939.852 | 1941.8 | 1940.2 | A |
| I-2607 | 904 | | >>20 | | 2015.884 | 2017.9 | 2016.2 | A |
| I-2608 | 251 | | | | 1863.821 | 1865.9 | 1864.1 | A |
| I-2609 | 2651 | | | | 1905.868 | 1908 | 1906.2 | A |
| I-2610 | 215 | | | | 2039.899 | 2042.1 | 2040.2 | |
| I-2611 | 188 | | | | 2053.915 | 2056.1 | 2054.2 | |
| I-2612 | 85 | | | | 1991.884 | 1993.8 | 1991.7 | |
| I-2613 | 183 | | | | 2053.915 | 2056.1 | 2054.9 | |
| I-2614 | 97 | | | | 2053.915 | 2056.1 | 2054.3 | |
| I-2615 | 115 | | | | 2067.93 | 2070.1 | 2068.5 | |
| I-2616 | 175 | | | | 2067.93 | 2070.1 | 2068.3 | |
| I-2617 | 130 | | | | 1991.884 | 1993.7 | 1992.4 | |
| I-2618 | 77 | | | | 2005.899 | 2008.1 | 2006.3 | |
| I-2619 | 36 | | | | 2005.899 | 2008.2 | 2006.3 | |

Table E3. Certain peptides and compositions thereof as examples. Peptides are stapled unless indicated otherwise (among other things, the present disclosure also provides unstapled versions of such peptides, optionally protected with one or more protection group (e.g., protection of N-terminus, C-terminus, side chains, etc.), and intermediates thereof). As appreciated by those skilled in the art, stapling may provide more than one stereoisomers (e.g., E Z of double bonds and/or diastereomers), and a stereoisomer can be EE, EZ, ZE, or ZZ for a stapled peptide with two staples each independently comprising a double bond. In some embodiments, isomers (or combinations thereof) are listed separately (typically based on HPLC peaks in the order of elution: an earlier eluted peak is assigned a smaller ID number than each later eluted peaks (if any); for example, isomer composition 2 corresponding to a composition of a later eluting peak than isomer composition 1, isomer composition 3 corresponding to a composition of a later eluting peak than isomer composition 2, etc.; in some cases, a peak may contain more than one isomer; in some cases, isomers are not separated (or a single isomer), e.g., when there is one peak on HPLC (e.g., reverse phase HPLC as described in Examples above)). Compositions utilized in various assays are typically of stapled peptides; the present disclosure also provides peptides prior to stapling and compositions thereof. In some embodiments, a HPLC method is as follows: Xselect CSH C18 column 1.7 um 2.1×50 mm 130 A; Column temperature 40° C.; Flow 0.6 mL/min; 0.1% formic acid in both acetonitrile and water, 7.2 min gradient from 5 to 95% acetonitrile. In some embodiments, a different gradient and/or a C8 column may be used.

| | | SEQ |
|---|---|---|
| | | ID |
| ID | Description | NO: |
| I-1 | Ac-Phe-Asp-Ala-RdN-Asp-Asp-Ala-Ala-Phe-Val-S7-Phe-Trp-Gln-NH2 | 7 |
| I-2 | Ac-Phe-Asp-Ala-RdN-Asp-Asp-Ala-Ala-Phe-Val-S7-Phe-Trp-Gln-NH2 | 7 |
| I-3 | Ac-Phe-Asp-Ala-R8-Asp-Glu-Ala-Ala-Phe-Val-PyrS-Phe-Trp-Gln-NH2 | 8 |
| I-4 | Ac-Phe-Asp-Ala-R8-Asp-Glu-Ala-Ala-Phe-Val-PyrS-Phe-Trp-Gln-NH2 | 8 |
| I-5 | Ac-Phe-Asp-Ala-R8-Asp-Aad-Ala-Ala-Phe-Val-PyrS-Phe-Trp-Gln-NH2 | 9 |
| I-6 | Ac-Phe-Asp-Ala-R8-Asp-Aad-Ala-Ala-Phe-Val-PyrS-Phe-Trp-Gln-NH2 | 9 |
| I-7 | Ac-Phe-Asp-Ala-R8-Asp-4COOHF-Ala-Ala-Phe-Val-PyrS-Phe-Trp-Gln-NH2 | 10 |
| I-8 | Ac-Phe-Asp-Ala-R8-Asp-4COOHF-Ala-Ala-Phe-Val-PyrS-Phe-Trp-Gln-NH2 | 10 |
| I-9 | Ac-Phe-Asp-Ala-R8-Asp-3PyrA-Ala-Ala-Phe-Val-PyrS-Phe-Trp-Gln-NH2 | 11 |
| I-10 | Ac-Phe-Asp-Ala-R8-Asp-3PyrA-Ala-Ala-Phe-Val-PyrS-Phe-Trp-Gln-NH2 | 11 |
| I-11 | Ac-Phe-Asp-Ala-R8-Asp-Bip-Ala-Ala-Phe-Val-PyrS-Phe-Trp-Gln-NH2 | 12 |
| I-12 | Ac-Phe-Asp-Ala-R8-Asp-Bip-Ala-Ala-Phe-Val-PyrS-Phe-Trp-Gln-NH2 | 12 |
| I-13 | Ac-Phe-Asp-Ala-R8-Asp-nLeu-Ala-Ala-Phe-Val-PyrS-Phe-Trp-Gln-NH2 | 13 |
| I-14 | Ac-Phe-Asp-Ala-R8-Asp-nLeu-Ala-Ala-Phe-Val-PyrS-Phe-Trp-Gln-NH2 | 13 |
| I-15 | Ac-Phe-Asp-Ala-R8-Asp-Cha-Ala-Ala-Phe-Val-PyrS-Phe-Trp-Gln-NH2 | 14 |
| I-16 | Ac-Phe-Asp-Ala-R8-Asp-Cha-Ala-Ala-Phe-Val-PyrS-Phe-Trp-Gln-NH2 | 14 |
| I-17 | Ac-Phe-Asp-Ala-R8-Asp-hLeu-Ala-Ala-Phe-Val-PyrS-Phe-Trp-Gln-NH2 | 15 |
| I-18 | Ac-Phe-Asp-Ala-R8-Asp-hLeu-Ala-Ala-Phe-Val-PyrS-Phe-Trp-Gln-NH2 | 15 |
| I-19 | Ac-Phe-Asp-Ala-R8-Asp-Asp-Ala-Asp-Phe-Val-PyrS-Phe-Trp-Gln-NH2 | 16 |
| I-20 | Ac-Phe-Asp-Ala-R8-Asp-Asp-Ala-Asp-Phe-Val-PyrS-Phe-Trp-Gln-NH2 | 16 |
| I-21 | Ac-Phe-Asp-Ala-R8-Asp-Asp-Ala-Glu-Phe-Val-PyrS-Phe-Trp-Gln-NH2 | 17 |
| I-22 | Ac-Phe-Asp-Ala-R8-Asp-Asp-Ala-Glu-Phe-Val-PyrS-Phe-Trp-Gln-NH2 | 17 |
| I-23 | Ac-Phe-Asp-Ala-R8-Asp-Asp-Ala-Aad-Phe-Val-PyrS-Phe-Trp-Gln-NH2 | 18 |
| I-24 | Ac-Phe-Asp-Ala-R8-Asp-Asp-Ala-Aad-Phe-Val-PyrS-Phe-Trp-Gln-NH2 | 18 |
| I-25 | Ac-Phe-Asp-Ala-R5-Asp-Asp-Ala-Ala-Phe-Val-SeN-Phe-Trp-Gln-NH2 | 19 |
| I-26 | Ac-Phe-Asp-Ala-R6-Asp-Asp-Ala-Ala-Phe-Val-SeN-Phe-Trp-Gln-NH2 | 20 |
| I-27 | Ac-Phe-Asp-Ala-ReN-Asp-Asp-Ala-Ala-Phe-Val-S5-Phe-Trp-Gln-NH2 | 21 |
| I-28 | Ac-Phe-Asp-Ala-ReN-Asp-Asp-Ala-Ala-Phe-Val-S6-Phe-Trp-Gln-NH2 | 22 |
| I-29 | Ac-Phe-Asp-Ala-ReN-Asp-Asp-Ala-Ala-Phe-Val-S6-Phe-Trp-Gln-NH2 | 22 |
| I-30 | Ac-Phe-Asp-Ala-R7-Asp-Asp-Ala-Ala-Phe-Val-PyrS-Phe-Trp-Gln-NH2 | 23 |
| I-31 | Ac-Phe-Asp-Ala-R7-Asp-Asp-Ala-Ala-Phe-Val-PyrS-Phe-Trp-Gln-NH2 | 23 |
| I-32 | Ac-Phe-Asp-Ala-Az-Asp-Asp-Ala-Ala-Phe-Val-S7-Phe-Trp-Gln-NH2 | 24 |
| I-33 | Ac-Phe-Asp-Ala-Az-Asp-Asp-Ala-Ala-Phe-Val-S7-Phe-Trp-Gln-NH2 | 24 |
| I-34 | Ac-Phe-Asp-Ala-R8-Asp-Asp-Ala-Ala-Phe-Val-SgN-Phe-Trp-Gln-NH2 | 25 |
| I-35 | Ac-Phe-Asp-Ala-Az-Asp-Asp-Ala-Ala-Phe-Val-S8-Phe-Trp-Gln-NH2 | 26 |
| I-36 | Ac-Phe-Asp-Ala-R4-Asp-Asp-Ala-Ala-Phe-Val-SeN-Phe-Trp-Gln-NH2 | 27 |
| I-37 | Ac-Phe-Asp-Ala-R5-Asp-Asp-Ala-Ala-Phe-Val-SdN-Phe-Trp-Gln-NH2 | 28 |
| I-38 | Ac-Phe-Asp-Ala-R7-Asp-Asp-Ala-Ala-Phe-Val-Az-Phe-Trp-Gln-NH2 | 29 |
| I-39 | Ac-Phe-Asp-Ala-R8-Asp-Asp-Ala-Ala-Phe-Val-Az-Phe-Trp-Gln-NH2 | 30 |
| I-40 | Ac-Phe-Asp-Ala-R8-Asp-Asp-Ala-Ala-Phe-Val-Az-Phe-Trp-Gln-NH2 | 30 |
| I-41 | Ac-Phe-Asp-Ala-RdN-Asp-Asp-Ala-Ala-Phe-Val-S4-Phe-Trp-Gln-NH2 | 31 |
| I-42 | Ac-Phe-Asp-Ala-RdN-Asp-Asp-Ala-Ala-Phe-Val-S4-Phe-Trp-Gln-NH2 | 31 |
| I-43 | Ac-Phe-Asp-Ala-RgN-Asp-Asp-Ala-Ala-Phe-Val-S8-Phe-Trp-Gln-NH2 | 32 |
| I-44 | Ac-Phe-Asp-Ala-RgN-Asp-Asp-Ala-Ala-Phe-Val-S8-Phe-Trp-Gln-NH2 | 32 |
| I-45 | Ac-Phe-Asp-Ala-RgN-Asp-Asp-Ala-Ala-Phe-Val-S7-Phe-Trp-Gln-NH2 | 33 |
| I-46 | Ac-Phe-Asp-Ala-RgN-Asp-Asp-Ala-Ala-Phe-Val-S7-Phe-Trp-Gln-NH2 | 33 |
| I-47 | Ac-Phe-Asp-R8-Ala-Asp-Asp-Ala-Ala-Phe-S5-Ala-Phe-Trp-Gln-NH2 | 34 |
| I-48 | Ac-Phe-Asp-R8-Ala-Asp-Asp-Ala-Ala-Phe-S5-Ala-Phe-Trp-Gln-NH2 | 34 |
| I-49 | Ac-Phe-Asp-RdN-Ala-Asp-Asp-Ala-Ala-Phe-S7-Ala-Phe-Trp-Gln-NH2 | 35 |
| I-50 | Ac-Phe-Asp-RdN-Ala-Asp-Asp-Ala-Ala-Phe-S7-Ala-Phe-Trp-Gln-NH2 | 35 |
| I-51 | Ac-Phe-Asp-R8-Ala-Asp-Asp-Ala-Ala-Phe-PyrS-Ala-Phe-Trp-Gln-NH2 | 36 |
| I-52 | Ac-PL3-Asp-Ala-B5-Asp-Asp-Ala-Ala-Phe-Val-S8-Phe-Trp-Gln-NH2 | 37 |
| I-53 | Ac-PL3-Asp-Ala-B5-Asp-Asp-Ala-Ala-Phe-Val-SeN-Phe-Trp-Gln-NH2 | 38 |
| I-54 | Ac-PL3-Asp-Ala-B5-Asp-Asp-Ala-Ala-Phe-Val-SeN-Phe-Trp-Gln-NH2 | 38 |
| I-55 | Ac-PL3-Asp-Ala-B5-Asp-Asp-Ala-Ala-Phe-Val-SdN-Phe-Trp-Gln-NH2 | 39 |
| I-56 | Ac-Phe-Asp-Ala-R8-Asp-aMeDF-Ala-Ala-Phe-Val-PyrS-Phe-Trp-Gln-NH2 | 40 |
| I-57 | Ac-Phe-Asp-Ala-R8-Asp-aMeDF-Ala-Ala-Phe-Val-PyrS-Phe-Trp-Gln-NH2 | 40 |
| I-58 | Ac-Phe-Asp-Ala-R8-Asp-Gln-Ala-Ala-Phe-Val-PyrS-Phe-Trp-Gln-NH2 | 41 |
| I-59 | Ac-Phe-Asp-Ala-R8-Asp-Gln-Ala-Ala-Phe-Val-PyrS-Phe-Trp-Gln-NH2 | 41 |
| I-60 | Ac-Phe-Asp-Ala-R8-Asp-Asp-Ala-Ala-Phe-Val-PyrS-Cha-Trp-Gln-NH2 | 42 |
| I-61 | Ac-Phe-Asp-Ala-R8-Asp-Asp-Ala-Ala-Phe-Val-PyrS-Cha-Trp-Gln-NH2 | 42 |
| I-62 | Ac-Phe-Asp-Ala-R8-Asp-Asp-Ala-Ala-Phe-Val-PyrS-Tyr-Trp-Gln-NH2 | 43 |
| I-63 | Ac-Phe-Asp-Ala-R8-Asp-Asp-Ala-Ala-Phe-Val-PyrS-Tyr-Trp-Gln-NH2 | 43 |
| I-64 | Ac-Ala-Asp-Ile-RdN-Asp-Asp-Ala-Ala-Phe-Gln-S7-Phe-Trp-His-NH2 | 44 |
| I-65 | Ac-Ala-Asp-Ile-RdN-Asp-Asp-Ala-Ala-Phe-Gln-S7-Phe-Trp-His-NH2 | 44 |
| I-66 | Ac-Ala-Asp-Ile-RdN-Asp-Glu-Ala-Ala-Phe-Gln-S7-Phe-Trp-His-NH2 | 45 |
| I-67 | Ac-Ala-Asp-Ile-RdN-Asp-Glu-Ala-Ala-Phe-Gln-S7-Phe-Trp-His-NH2 | 45 |
| I-68 | Ac-Ala-Asp-Ile-RdN-Asp-nLeu-Ala-Ala-Phe-Gln-S7-Phe-Trp-His-NH2 | 46 |
| I-69 | Ac-Ala-Asp-Ile-RdN-Asp-nLeu-Ala-Ala-Phe-Gln-S7-Phe-Trp-His-NH2 | 46 |
| I-70 | Ac-Ala-Asp-Ile-RdN-Asp-hLeu-Ala-Ala-Phe-Gln-S7-Phe-Trp-His-NH2 | 47 |
| I-71 | Ac-Ala-Asp-Ile-RdN-Asp-hLeu-Ala-Ala-Phe-Gln-S7-Phe-Trp-His-NH2 | 47 |
| I-72 | Ac-Ala-Asp-Ile-RdN-Asp-Cha-Ala-Ala-Phe-Gln-S7-Phe-Trp-His-NH2 | 48 |
| I-73 | Ac-Ala-Asp-Ile-RdN-Asp-Cha-Ala-Ala-Phe-Gln-S7-Phe-Trp-His-NH2 | 48 |
| I-74 | Ac-Phe-Asp-Ala-R8-Asp-Asp-Ala-Ala-Cha-Val-PyrS-Phe-Trp-Gln-NH2 | 49 |
| I-75 | Ac-Phe-Asp-Ala-R8-Asp-Asp-Ala-Ala-Cha-Val-PyrS-Phe-Trp-Gln-NH2 | 49 |
| I-76 | Ac-Phe-Asp-Ala-R8-Asp-Asp-Ala-Ala-nLeu-Val-PyrS-Phe-Trp-Gln-NH2 | 50 |
| I-77 | Ac-Phe-Asp-Ala-R8-Asp-Asp-Ala-Ala-3FF-Val-PyrS-Phe-Trp-Gln-NH2 | 51 |
| I-78 | Ac-Phe-Asp-Ala-R8-Asp-Asp-Ala-Ala-4FF-Val-PyrS-Phe-Trp-Gln-NH2 | 52 |
| I-79 | Ac-Phe-Asp-Ala-R8-Asp-Asp-Ala-Ala-34FF-Val-PyrS-Phe-Trp-Gln-NH2 | 53 |
| I-80 | Ac-Phe-Asp-Ala-R8-Asp-Asp-Ala-Ala-Phe-Val-PyrS-2PyrA-Trp-Gln-NH2 | 54 |
| I-81 | Ac-Phe-Asp-Ala-R8-Asp-Asp-Ala-Ala-Phe-Val-PyrS-2PyrA-Trp-Gln-NH2 | 54 |
| I-82 | Ac-Phe-Asp-Ala-R8-Asp-Asp-Ala-Ala-Phe-Val-PyrS-3PyrA-Trp-Gln-NH2 | 55 |
| I-83 | Ac-Phe-Asp-Ala-R8-Asp-Asp-Ala-Ala-Phe-Val-PyrS-3PyrA-Trp-Gln-NH2 | 55 |
| I-84 | Ac-Phe-Asp-Ala-R8-Asp-Asp-Ala-Ala-Phe-Val-PyrS-4PyrA-Trp-Gln-NH2 | 56 |
| I-85 | Ac-Phe-Asp-Ala-R8-Asp-Asp-Ala-Ala-Phe-Val-PyrS-4PyrA-Trp-Gln-NH2 | 56 |
| I-86 | Ac-Phe-Asp-Ala-R8-Asp-Asp-Ala-Ala-2PyrA-Val-PyrS-Phe-Trp-Gln-NH2 | 57 |
| I-87 | Ac-Phe-Asp-Ala-R8-Asp-Asp-Ala-Ala-3PyrA-Val-PyrS-Phe-Trp-Gln-NH2 | 58 |
| I-88 | Ac-Phe-Asp-Ala-R8-Asp-Asp-Ala-Ala-3PyrA-Val-PyrS-Phe-Trp-Gln-NH2 | 58 |
| I-89 | Ac-Phe-Asp-Ala-R8-Asp-Asp-Ala-Ala-Phe-Val-PyrS-4CBMF-Trp-Gln-NH2 | 59 |
| I-90 | Ac-Phe-Asp-Ala-R8-Asp-Asp-Ala-Ala-Phe-Val-PyrS-4CBMF-Trp-Gln-NH2 | 59 |
| I-91 | Ac-Phe-Asp-Ala-R8-Asp-Asp-Ala-Ala-4CBMF-Val-PyrS-Phe-Trp-Gln-NH2 | 60 |
| I-92 | Ac-Phe-Asp-Ala-R8-Asp-Asp-Ala-Ala-4CBMF-Val-PyrS-Phe-Trp-Gln-NH2 | 60 |
| I-93 | Ac-Phe-Asp-Ala-R8-Asp-Asp-Ala-Ala-Phe-Val-PyrS-CypA-Trp-Gln-NH2 | 61 |
| I-94 | Ac-Phe-Asp-Ala-R8-Asp-Asp-Ala-Ala-Phe-Val-PyrS-CypA-Trp-Gln-NH2 | 61 |
| I-95 | Ac-Phe-Asp-Ala-R8-Asp-Asp-Ala-Ala-CypA-Val-PyrS-Phe-Trp-Gln-NH2 | 62 |
| I-96 | Ac-Phe-Asp-Ala-R8-Asp-Asp-Ala-Ala-CypA-Val-PyrS-Phe-Trp-Gln-NH2 | 62 |
| I-97 | Ac-Phe-Asp-Ala-R8-Asp-hPhe-Ala-Ala-Phe-Val-PyrS-Phe-Trp-Gln-NH2 | 63 |
| I-98 | Ac-Phe-Asp-Ala-R8-Asp-hPhe-Ala-Ala-Phe-Val-PyrS-Phe-Trp-Gln-NH2 | 63 |
| I-99 | Ac-Phe-Asp-Ala-R8-Asp-Phe-Ala-Ala-Phe-Val-PyrS-Phe-Trp-Gln-NH2 | 64 |
| I-100 | Ac-Phe-Asp-Ala-R8-Asp-Phe-Ala-Ala-Phe-Val-PyrS-Phe-Trp-Gln-NH2 | 64 |
| I-101 | Ac-Phe-Asp-Ala-R8-Asp-Asp-Ala-Ala-Phe-Val-PyrS-3OMeF-Trp-Gln-NH2 | 65 |
| I-102 | Ac-Phe-Asp-Ala-R8-Asp-Asp-Ala-Ala-Phe-Val-PyrS-3OMeF-Trp-Gln-NH2 | 65 |
| I-103 | Ac-Phe-Asp-Ala-R8-Asp-Asp-Ala-Ala-3OMeF-Val-PyrS-Phe-Trp-Gln-NH2 | 66 |
| I-104 | Ac-Phe-Asp-Ala-R8-Asp-Asp-Ala-Ala-3OMeF-Val-PyrS-Phe-Trp-Gln-NH2 | 66 |
| I-105 | Ac-Phe-Asp-Ala-R8-Asp-Asp-Ala-Ala-Phe-Val-PyrS-2CBMF-Trp-Gln-NH2 | 67 |
| I-106 | Ac-Phe-Asp-Ala-R8-Asp-Asp-Ala-Ala-Phe-Val-PyrS-2CBMF-Trp-Gln-NH2 | 67 |
| I-107 | Ac-Phe-Asp-Ala-R8-Asp-Asp-Ala-Ala-Phe-Val-PyrS-3CBMF-Trp-Gln-NH2 | 68 |
| I-108 | Ac-Phe-Asp-Ala-R8-Asp-Asp-Ala-Ala-3CBMF-Val-PyrS-Phe-Trp-Gln-NH2 | 69 |
| I-109 | Ac-Phe-Asp-Ala-R8-Asp-Asp-Ala-Ala-3CBMF-Val-PyrS-Phe-Trp-Gln-NH2 | 69 |
| I-110 | Ac-Phe-Asp-Ala-R8-Asp-Asp-Ala-Ala-Phe-Val-PyrS-4COOHF-Trp-Gln-NH2 | 70 |
| I-111 | Ac-Phe-Asp-Ala-R8-Asp-Asp-Ala-Ala-Phe-Val-PyrS-4COOHF-Trp-Gln-NH2 | 70 |
| I-112 | Ac-Phe-Asp-Ala-R8-Asp-Asp-Ala-Ala-2NapA-Val-PyrS-Phe-Trp-Gln-NH2 | 71 |
| I-113 | Ac-Phe-Asp-Ala-R8-Asp-Asp-Ala-Ala-2NapA-Val-PyrS-Phe-Trp-Gln-NH2 | 71 |
| I-114 | Ac-Phe-Asp-Ala-R8-Asp-1NapA-Ala-Ala-Phe-Val-PyrS-Phe-Trp-Gln-NH2 | 72 |
| I-115 | Ac-Phe-Asp-Ala-R8-Asp-2NapA-Ala-Ala-Phe-Val-PyrS-Phe-Trp-Gln-NH2 | 73 |
| I-116 | Ac-Phe-Asp-Ala-R8-Asp-2NapA-Ala-Ala-Phe-Val-PyrS-Phe-Trp-Gln-NH2 | 73 |
| I-117 | Ac-Phe-Asp-Ala-R8-Asp-Asp-Ala-Ala-hPhe-Val-PyrS-Phe-Trp-Gln-NH2 | 74 |
| I-118 | Ac-Phe-Asp-Ala-R8-Asp-Asp-Ala-Ala-hPhe-Val-PyrS-Phe-Trp-Gln-NH2 | 74 |
| I-119 | Ac-Phe-Asp-Ala-R8-Asp-Asp-Ala-Ala-hTyr-Val-PyrS-Phe-Trp-Gln-NH2 | 75 |
| I-120 | Ac-Phe-Asp-Ala-R8-Asp-Asp-Ala-Ala-hTyr-Val-PyrS-Phe-Trp-Gln-NH2 | 75 |
| I-121 | Ac-Phe-Asp-Ala-R8-Asp-Asp-Ala-Ala-Phe-Val-PyrS-Asp-Trp-Gln-NH2 | 76 |
| I-122 | Ac-Phe-Asp-Ala-R8-Asp-Asp-Ala-Ala-Phe-Val-PyrS-Asp-Trp-Gln-NH2 | 76 |
| I-123 | Ac-PL3-Asp-Ala-B5-Asp-Glu-Ala-Ala-Phe-Val-S8-Phe-Trp-Gln-NH2 | 77 |
| I-124 | Ac-PL3-Asp-Ala-B5-Asp-Asn-Ala-Ala-Phe-Val-S8-Phe-Trp-Gln-NH2 | 78 |
| I-125 | Ac-PL3-Asp-Ala-B5-Asp-Asn-Ala-Ala-Phe-Val-S8-Phe-Trp-Gln-NH2 | 78 |
| I-126 | Ac-PL3-Asp-Ala-B5-Asp-Gln-Ala-Ala-Phe-Val-S8-Phe-Trp-Gln-NH2 | 79 |
| I-127 | Ac-PL3-Asp-Ala-B5-Asp-Gln-Ala-Ala-Phe-Val-S8-Phe-Trp-Gln-NH2 | 79 |
| I-128 | Ac-PL3-Asp-Ala-B5-Asp-Glu-Ala-Ala-Phe-Val-S8-Phe-Trp-NH2 | 80 |
| I-129 | Ac-PL3-Asp-Ala-B5-Asp-Aad-Ala-Ala-Phe-Val-S8-Phe-Trp-Gln-NH2 | 81 |
| I-130 | Ac-PL3-Asp-Ala-B5-Asp-Glu-Ala-Ala-Phe-Val-SeN-Phe-Trp-Gln-NH2 | 82 |
| I-131 | Ac-PL3-Asp-Ala-B5-Asp-Glu-Ala-Ala-Phe-Val-SeN-Phe-Trp-Gln-NH2 | 82 |
| I-132 | Ac-PL3-Asp-Ala-B5-Asp-Glu-Ala-Ala-Phe-Val-S7-Phe-Trp-Gln-NH2 | 83 |
| I-133 | Ac-PL3-Asp-Ala-B5-Asp-Glu-Ala-Ala-Phe-Val-S8-Phe-Trp-Aib-NH2 | 84 |
| I-134 | Ac-PL3-Asp-Ala-B5-Asp-Glu-Ala-Ala-Phe-Val-S8-Phe-Trp-Aib-NH2 | 84 |
| I-135 | Ac-PL3-Asp-Ala-B5-Asp-Glu-Ala-Ala-Phe-Val-S8-Phe-Trp-Ala-NH2 | 85 |
| I-136 | Ac-PL3-Asp-Ala-B5-Asp-Glu-Ala-Ala-Phe-Val-S8-Phe-aMeW-Gln-NH2 | 86 |
| I-137 | Ac-PL3-Asp-Ala-B5-Asp-Glu-Ala-Ala-Phe-Val-S8-Phe-aMeW-Gln-NH2 | 86 |
| I-138 | Ac-PL3-Asp-Ala-B5-Asp-nLeu-Ala-Ala-Phe-Val-S8-Phe-Trp-Gln-NH2 | 87 |
| I-139 | Ac-PL3-Asp-Ala-B5-Asp-nLeu-Ala-Ala-Phe-Val-S8-Phe-Trp-Gln-NH2 | 87 |
| I-140 | Ac-PL3-Asp-Ala-B5-Asp-Glu-Ala-Ala-Phe-Val-S8-Phe-2NapA-Gln-NH2 | 88 |
| I-141 | Ac-PL3-Asp-Ala-B5-Asp-Glu-Ala-Ala-Phe-Val-S8-Phe-2NapA-NH2 | 89 |
| I-142 | Ac-PL3-Asp-Ala-B5-Asp-Glu-Ala-Ala-Phe-Val-S8-Phe-6F1NapA-Gln-NH2 | 90 |
| I-143 | Ac-PL3-Asp-Ala-B5-Asp-Glu-Ala-Ala-Phe-Val-S8-Phe-Qui-Gln-NH2 | 91 |
| I-144 | Ac-PL3-Asp-Ala-B5-Asp-Glu-Ala-Ala-Phe-Val-S8-Phe-BztA-Gln-NH2 | 92 |
| I-145 | Ac-PL3-Asp-Ala-B5-Asp-Glu-Ala-Ala-Phe-Val-S8-Phe-Bip-Gln-NH2 | 93 |
| I-146 | Ac-PL3-Asp-Ala-B5-Asp-Glu-Ala-Ala-Phe-Val-S8-Phe-5FW-Gln-NH2 | 94 |
| I-147 | Ac-Aib-Asp-Ile-RdN-Asp-Glu-Ala-Ala-Phe-Gln-S7-Phe-Trp-NH2 | 95 |
| I-148 | Ac-Ala-Asp-Ile-RdN-Asp-Glu-Ala-Ala-Phe-Gln-S7-Phe-Trp-NH2 | 96 |
| I-149 | Ac-Asp-Ile-RdN-Asp-Asp-Ala-Ala-Phe-Gln-S7-Phe-Trp-His-NH2 | 97 |
| I-150 | Ac-Asp-Ile-RdN-Asp-Asp-Ala-Ala-Phe-Gln-S7-Phe-Trp-His-NH2 | 97 |
| I-151 | Ac-Asp-Ile-RdN-Asp-Asp-Ala-Ala-Phe-Gln-S7-Phe-Trp-NH2 | 98 |
| I-152 | Ac-Ala-Asp-Ile-RdN-Asp-Glu-Ala-Ala-Phe-Ala-S7-Phe-Trp-His-NH2 | 99 |
| I-153 | Ac-Ala-Asp-Ile-RdN-Asp-Glu-Ala-Ala-Phe-Gln-S7-Phe-Trp-Ala-NH2 | 100 |
| I-154 | Ac-Ala-Asp-Ile-RdN-Asp-Glu-Ala-Ala-Phe-Ala-S7-Phe-Trp-Ala-NH2 | 101 |
| I-155 | Ac-Ala-Asp-Ile-RdN-Asp-Glu-Ala-Ala-Phe-Ala-S7-Phe-Trp-Ala-NH2 | 101 |
| I-156 | Ac-Ala-Asp-Ala-RdN-Asp-Glu-Ala-Ala-Phe-Gln-S7-Phe-Trp-His-NH2 | 102 |
| I-157 | Ac-Aib-Asp-Ala-R8-Asp-Glu-Ala-Ala-Phe-Val-PyrS-Phe-Trp-Gln-NH2 | 103 |
| I-158 | Ac-Aib-Asp-Ala-R8-Asp-Glu-Ala-Ala-Phe-Val-PyrS-Phe-Trp-Gln-NH2 | 103 |
| I-159 | Ac-Aib-Asp-Ala-R8-Asp-Glu-Ala-Ala-Phe-Val-PyrS-Phe-Trp-Aib-NH2 | 104 |
| I-160 | Ac-Aib-Asp-Ala-R8-Asp-Glu-Ala-Ala-Phe-Val-PyrS-Phe-Trp-Aib-NH2 | 104 |
| I-161 | Ac-Phe-Asp-Ala-R8-Asp-Asp-Ala-Ala-Phe-Val-PyrS-Glu-Trp-Gln-NH2 | 105 |
| I-162 | Ac-Phe-Asp-Ala-R8-Asp-Asp-Ala-Ala-Phe-Val-PyrS-Glu-Trp-Gln-NH2 | 105 |
| I-163 | Ac-Phe-Asp-Ala-R8-Asp-Asp-Ala-Ala-Phe-Ser-PyrS-Phe-Trp-Gln-NH2 | 106 |
| I-164 | Ac-Phe-Asp-Ala-R8-Asp-Asp-Ala-Ala-Phe-Ser-PyrS-Phe-Trp-Gln-NH2 | 106 |
| I-165 | Ac-PL3-Asp-Ala-B5-Asp-Glu-Ala-Ala-Phe-Val-S8-Phe-5MeOW-Gln-NH2 | 107 |
| I-166 | Ac-PL3-Asp-Ala-B5-Asp-Glu-Ala-Ala-Phe-Val-S8-Phe-7FW-Gln-NH2 | 108 |
| I-167 | Ac-PL3-Asp-Ala-B5-Asp-Glu-Ala-Ala-Phe-Val-S8-Phe-H2Trp-Gln-NH2 | 109 |
| I-168 | Ac-PL3-Asp-Ala-B5-Asp-Glu-Ala-Ala-Phe-Val-S8-Phe-H2Trp-Gln-NH2 | 109 |
| I-169 | Ac-PL3-Asp-Ala-B5-Asp-Glu-Ala-Ala-Phe-Val-S8-Phe-5CF3W-Gln-NH2 | 110 |
| I-170 | Ac-PL3-Asp-Ala-B5-Asp-Glu-Ala-Ala-Phe-Val-S8-Phe-5CpW-Gln-NH2 | 111 |
| I-171 | Ac-PL3-Asp-Ala-B5-Asp-Glu-Ala-Ala-Phe-Val-S8-Phe-5CpW-Gln-NH2 | 111 |
| I-172 | Ac-PL3-Asp-Ala-B5-Asp-EtGA-Ala-Ala-Phe-Val-S8-Phe-Trp-Gln-NH2 | 112 |
| I-173 | Ac-PL3-Asp-Ala-B5-Asp-EtGA-Ala-Ala-Phe-Val-S8-Phe-Trp-Gln-NH2 | 112 |
| I-174 | Ac-PL3-Asp-Ile-B5-Asp-Asp-Ala-Ala-Phe-Gln-S8-Phe-Trp-His-NH2 | 113 |
| I-175 | Ac-PL3-Asp-Ile-B5-Asp-Asp-Ala-Ala-Phe-Gln-S8-Phe-Trp-His-NH2 | 113 |
| I-176 | Ac-PL3-Asp-Ile-B5-Asp-Glu-Ala-Ala-Phe-Val-S8-Phe-Trp-His-NH2 | 114 |
| I-177 | Ac-PL3-Asp-Ile-B5-Asp-Glu-Ala-Ala-Phe-Val-S8-Phe-Trp-His-NH2 | 114 |
| I-178 | Ac-PL3-Asp-Ala-B5-Asp-Glu-Ala-Ala-Phe-Gln-S8-Phe-Trp-His-NH2 | 115 |
| I-179 | Ac-PL3-Asp-Ala-B5-Asp-Glu-Ala-Ala-Phe-Gln-S8-Phe-Trp-His-NH2 | 115 |
| I-180 | Ac-PL3-Asp-Ile-B5-Asp-Glu-Ala-Ala-Phe-Gln-S8-Phe-Trp-Gln-NH2 | 116 |
| I-181 | Ac-PL3-Asp-Ile-B5-Asp-Glu-Ala-Ala-Phe-Gln-S8-Phe-Trp-Gln-NH2 | 116 |
| I-182 | Ac-PL3-Asp-Ala-B5-Asp-DGlu-Ala-Ala-Phe-Val-S8-Phe-Trp-Gln-NH2 | 117 |
| I-183 | Ac-PL3-Asp-Ala-B5-Asp-Glu-Ala-Ala-Phe-Val-S8-Pff-Trp-Gln-NH2 | 118 |
| I-184 | Ac-PL3-Asp-Ala-B5-Asp-Glu-Ala-Ala-34FF-Val-S8-Phe-2NapA-Gln-NH2 | 119 |
| I-185 | Ac-PL3-Asp-Ala-B5-Asp-Glu-Ala-Ala-34FF-Ala-S8-Phe-2NapA-Ala-NH2 | 120 |
| I-186 | Ac-PL3-Asp-Ala-B5-Asp-Glu-Ala-Ala-Phe-Ala-S8-Phe-Trp-Ala-NH2 | 121 |
| I-187 | Ac-PL3-Asp-Ala-B5-Asp-Glu-Ala-Ala-Phe-Ala-S8-Phe-Trp-Ala-NH2 | 121 |
| I-188 | Ac-PL3-Asp-Ile-B5-Asp-Asp-Ala-Ala-Phe-Gln-SeN-Phe-Trp-His-NH2 | 122 |
| I-189 | Ac-PL3-Asp-Ile-B5-Asp-Asp-Ala-Ala-Phe-Gln-SeN-Phe-Trp-His-NH2 | 122 |
| I-190 | Ac-PL3-Asp-Ile-B5-Asp-Asp-Ala-Ala-Phe-Gln-SeN-Phe-Trp-His-NH2 | 122 |
| I-191 | Ac-PL3-Asp-Ile-B5-Asp-Glu-Ala-Ala-Phe-Gln-S8-Phe-Trp-NH2 | 123 |
| I-192 | Ac-PL3-Asp-Ile-B5-Asp-Glu-Ala-Ala-Phe-Gln-S8-Phe-Trp-NH2 | 123 |
| I-193 | Ac-PL3-Asp-Ile-B5-Asp-Glu-Ala-Ala-Phe-Gln-S8-Phe-Trp-NH2 | 123 |
| I-194 | Ac-PL3-Asp-Ile-B5-Asp-Glu-Ala-Ala-Phe-Gln-S8-Phe-Trp-Ala-NH2 | 124 |
| I-195 | Ac-PL3-Asp-Ala-B5-Asp-hPhe-Ala-Ala-Phe-Val-S8-Phe-Trp-Gln-NH2 | 125 |
| I-196 | Ac-PL3-Asp-Ala-B5-hPhe-Glu-Ala-Ala-Phe-Val-S8-Phe-Trp-Gln-NH2 | 126 |
| I-197 | Ac-PL3-Asp-Ala-B5-hPhe-Glu-Ala-Ala-Phe-Val-S8-Phe-Trp-Gln-NH2 | 126 |
| I-198 | Ac-PL3-Asp-Ala-B5-Asn-Glu-Ala-Ala-Phe-Val-S8-Phe-Trp-Gln-NH2 | 127 |
| I-199 | Ac-PL3-Asn-Ala-B5-Asp-Glu-Ala-Ala-Phe-Val-S8-Phe-Trp-Gln-NH2 | 128 |
| I-200 | Ac-PL3-Asn-Ala-B5-Asp-Glu-Ala-Ala-Phe-Val-S8-Phe-Trp-Gln-NH2 | 128 |
| I-201 | Ac-PL3-Phe-Ala-B5-Asp-Glu-Ala-Ala-Phe-Val-S8-Phe-Trp-Gln-NH2 | 129 |
| I-202 | Ac-PL3-Tyr-Ala-B5-Asp-Glu-Ala-Ala-Phe-Val-S8-Phe-Trp-Gln-NH2 | 130 |
| I-203 | Ac-PL3-3MeF-Ala-B5-Asp-Glu-Ala-Ala-Phe-Val-S8-Phe-Trp-Gln-NH2 | 131 |
| I-204 | Ac-PL3-3FF-Ala-B5-Asp-Glu-Ala-Ala-Phe-Val-S8-Phe-Trp-Gln-NH2 | 132 |
| I-205 | Ac-PL3-Asp-Ala-B5-nLeu-Glu-Ala-Ala-Phe-Val-S8-Phe-Trp-Gln-NH2 | 133 |
| I-206 | Ac-PL3-Asp-Ala-B5-His-Glu-Ala-Ala-Phe-Val-S8-Phe-Trp-Gln-NH2 | 134 |
| I-207 | Ac-PL3-Asp-Ala-B5-Asp-Glu-Ala-Ala-3MeF-Val-S8-Phe-Trp-Gln-NH2 | 135 |
| I-208 | Ac-PL3-Asp-Ala-B5-Asp-Glu-Ala-Ala-3MeF-Val-S8-Phe-Trp-Gln-NH2 | 135 |
| I-209 | Ac-PL3-Asp-Ala-B5-Asp-Glu-Ala-Ala-345FF-Val-S8-Phe-Trp-Gln-NH2 | 136 |
| I-210 | Ac-PL3-Asp-Ala-B5-Asp-Glu-Ala-Ala-345FF-Val-S8-Phe-Trp-Gln-NH2 | 136 |
| I-211 | Ac-PL3-Asp-Ala-B5-Asp-Glu-Ala-Ala-345FF-Val-S8-Phe-Trp-Gln-NH2 | 136 |
| I-212 | Ac-PL3-Asp-Ala-B5-Asp-Glu-Ala-Ala-345FF-Val-S8-Phe-Trp-Gln-NH2 | 136 |
| I-213 | Ac-PL3-Asp-Ala-B5-3pyrA-Glu-Ala-Ala-Phe-Val-S8-Phe-Trp-Gln-NH2 | 137 |
| I-214 | Ac-PL3-Asp-Ala-B5-4pyrA-Glu-Ala-Ala-Phe-Val-S8-Phe-Trp-Gln-NH2 | 138 |
| I-215 | Ac-PL3-Asp-Ala-B5-Asp-DaMeL-Ala-Ala-Phe-Val-S8-Phe-Trp-Gln-NH2 | 139 |
| I-216 | Ac-PL3-Asp-Ala-B5-Asp-DaMeL-Ala-Ala-Phe-Val-S8-Phe-Trp-Gln-NH2 | 139 |
| I-217 | Ac-PL3-Asp-Ala-B5-Asp-Glu-Ala-Ala-MeTyr-Val-S8-Phe-Trp-Gln-NH2 | 140 |
| I-218 | Ac-PL3-Asp-Ala-B5-Asp-Glu-Ala-Ala-MeTyr-Val-S8-Phe-Trp-Gln-NH2 | 140 |
| I-219 | Ac-PL3-Asp-Ala-B5-Asp-Glu-Ala-Ala-35FF-Val-S8-Phe-Trp-Gln-NH2 | 141 |
| I-220 | Ac-PL3-Asp-Ala-B5-Asp-Glu-Ala-Ala-35FF-Val-S8-Phe-Trp-Gln-NH2 | 141 |
| I-221 | Ac-Phe-Asp-Ala-R8-Asp-Asp-Ala-Ala-Phe-Thr-PyrS-Phe-Trp-Gln-NH2 | 142 |
| I-222 | Ac-Phe-Asp-Ala-R8-Asp-Asp-Ala-Ala-Phe-Thr-PyrS-Phe-Trp-Gln-NH2 | 142 |
| I-223 | Ac-Phe-Asp-Ala-R8-Asp-Asp-Ala-Ala-Phe-Thr-PyrS-Phe-Trp-Gln-NH2 | 142 |
| I-224 | Ac-Phe-Asp-Ala-R8-Asp-Asp-Ala-Ala-Phe-Thr-PyrS-Phe-Trp-Gln-NH2 | 142 |
| I-225 | Ac-Phe-Asp-Ala-R8-Asp-Asp-Ala-Ala-Phe-Val-PyrS-Pff-Trp-Gln-NH2 | 143 |
| I-226 | Ac-Phe-Asp-Ala-R8-Asp-Asp-Ala-Ala-Phe-Val-PyrS-Pff-Trp-Gln-NH2 | 143 |
| I-227 | Ac-Phe-Asp-Ala-R8-Asp-Asp-Ala-Ala-Phe-Val-PyrS-Pff-Trp-Gln-NH2 | 143 |
| I-228 | Ac-Phe-Asp-Ala-R8-Asp-Asp-Ala-Ala-Phe-Val-PyrS-Pff-Trp-Gln-NH2 | 143 |
| I-229 | Ac-PL3-Asp-Ala-B5-2pyrA-Glu-Ala-Ala-Phe-Val-S8-Phe-Trp-Gln-NH2 | 144 |
| I-230 | Ac-PL3-Asp-Ala-B5-2pyrA-Glu-Ala-Ala-Phe-Val-S8-Phe-Trp-Gln-NH2 | 144 |
| I-231 | Ac-PL3-Asp-Ala-B5-Asp-BztA-Ala-Ala-Phe-Val-S8-Phe-Trp-Gln-NH2 | 145 |
| I-232 | Ac-PL3-Asp-Ala-B5-Asp-2NapA-Ala-Ala-Phe-Val-S8-Phe-Trp-Gln-NH2 | 146 |
| I-233 | Ac-PL3-Asp-Ala-B5-Asp-Trp-Ala-Ala-Phe-Val-S8-Phe-Trp-Gln-NH2 | 147 |
| I-234 | Ac-PL3-Asp-Ala-B5-Asp-1NapA-Ala-Ala-Phe-Val-S8-Phe-Trp-Gln-NH2 | 148 |
| I-235 | Ac-PL3-Asp-Ala-B5-Asp-Bip-Ala-Ala-Phe-Val-S8-Phe-Trp-Gln-NH2 | 149 |
| I-236 | Ac-PL3-Asp-Ala-B5-Asp-Asp-Ala-Ala-3F3MeF-Val-S8-Phe-Trp-Gln-NH2 | 150 |
| I-237 | Ac-PL3-Asp-Ala-B5-Asp-Asp-Ala-Ala-3F3MeF-Val-S8-Phe-Trp-Gln-NH2 | 150 |
| I-238 | Ac-PL3-Asp-Ala-B5-Asp-Asp-Ala-Ala-3F3MeF-Val-S8-Phe-Trp-Gln-NH2 | 150 |
| I-239 | Ac-PL3-Asp-Ala-B5-Asp-Asp-Ala-Ala-3F3MeF-Val-S8-Phe-Trp-Gln-NH2 | 150 |
| I-240 | Ac-PL3-Asp-Ala-B5-Asp-Asp-Ala-Ala-4F3MeF-Val-S8-Phe-Trp-Gln-NH2 | 151 |
| I-241 | Ac-PL3-Asp-Ala-B5-Asp-Asp-Ala-Ala-4F3MeF-Val-S8-Phe-Trp-Gln-NH2 | 151 |
| I-242 | Ac-PL3-Asp-Ala-B5-Asp-Asp-Ala-Ala-4F3MeF-Val-S8-Phe-Trp-Gln-NH2 | 151 |
| I-243 | Ac-PL3-Asp-Ala-B5-Asp-Asp-Ala-Ala-4F3MeF-Val-S8-Phe-Trp-Gln-NH2 | 151 |
| I-244 | Ac-PL3-Asp-Ala-B5-Asp-3F3MeF-Ala-Ala-Phe-Val-S8-Phe-Trp-Gln-NH2 | 152 |
| I-245 | Ac-PL3-Asp-Ala-B5-Asp-3F3MeF-Ala-Ala-Phe-Val-S8-Phe-Trp-Gln-NH2 | 152 |
| I-246 | Ac-PL3-Asp-Ala-B5-Asp-4F3MeF-Ala-Ala-Phe-Val-S8-Phe-Trp-Gln-NH2 | 153 |
| I-247 | Ac-PL3-Asp-Ala-B5-Asp-4F3MeF-Ala-Ala-Phe-Val-S8-Phe-Trp-Gln-NH2 | 153 |
| I-248 | Ac-PL3-Asp-Ala-B5-Asp-Asp-Ala-Ala-Phe-Val-S8-3F3MeF-Trp-Gln-NH2 | 154 |
| I-249 | Ac-PL3-Asp-Ala-B5-Asp-Asp-Ala-Ala-Phe-Val-S8-3F3MeF-Trp-Gln-NH2 | 154 |
| I-250 | Ac-PL3-Asp-Ala-B5-Asp-Asp-Ala-Ala-Phe-Val-S8-3F3MeF-Trp-Gln-NH2 | 154 |
| I-251 | Ac-PL3-Asp-Ala-B5-Asp-Asp-Ala-Ala-Phe-Val-S8-3F3MeF-Trp-Gln-NH2 | 154 |
| I-252 | Ac-PL3-Asp-Ala-B5-Asp-Asp-Ala-Ala-Phe-Val-S8-4F3MeF-Trp-Gln-NH2 | 155 |
| I-253 | Ac-PL3-Asp-Ala-B5-Asp-Asp-Ala-Ala-Phe-Val-S8-4F3MeF-Trp-Gln-NH2 | 155 |
| I-254 | Ac-PL3-Asp-Ala-B5-Asp-Asp-Ala-Ala-Phe-Val-S8-4F3MeF-Trp-Gln-NH2 | 155 |
| I-255 | Ac-PL3-Asp-Ala-B5-Asp-Asp-Ala-Ala-Phe-Val-S8-4F3MeF-Trp-Gln-NH2 | 155 |
| I-256 | Ac-PL3-Asp-Ala-B5-Asp-6F1NapA-Ala-Ala-Phe-Val-S8-Phe-Trp-Gln-NH2 | 156 |
| I-257 | Ac-PL3-Asp-Ala-B5-Asp-3FF-Ala-Ala-Phe-Val-S8-Phe-Trp-Gln-NH2 | 157 |
| I-258 | Ac-PL3-Asp-Ala-B5-Asp-4FF-Ala-Ala-Phe-Val-S8-Phe-Trp-Gln-NH2 | 158 |
| I-259 | Ac-PL3-Asp-Ala-B5-Asp-34FF-Ala-Ala-Phe-Val-S8-Phe-Trp-Gln-NH2 | 159 |
| I-260 | Ac-PL3-Asp-Ala-B5-Asp-Asp-Ala-Ala-Phe-Val-S8-3FF-Trp-Gln-NH2 | 160 |
| I-261 | Ac-PL3-Asp-Ala-B5-Asp-Asp-Ala-Ala-Phe-Val-S8-4FF-Trp-Gln-NH2 | 161 |
| I-262 | Ac-PL3-Asp-Ala-B5-Asp-Asp-Ala-Ala-Phe-Val-S8-34FF-Trp-Gln-NH2 | 162 |
| I-263 | Ac-PL3-Asp-Ala-B5-Asp-Asp-Ala-Ala-Phe-Val-S8-34FF-Trp-Gln-NH2 | 162 |
| I-264 | Ac-PL3-Asp-Ala-B5-Asp-Asp-Ala-Ala-3FF-Val-S8-Phe-Trp-Gln-NH2 | 163 |
| I-265 | Ac-PL3-Asp-Ala-B5-Asp-Asp-Ala-Ala-3FF-Val-S8-Phe-Trp-Gln-NH2 | 163 |
| I-266 | Ac-PL3-Asp-Ala-B5-Asp-Asp-Ala-Ala-4FF-Val-S8-Phe-Trp-Gln-NH2 | 164 |
| I-267 | Ac-PL3-Asp-Ala-B5-Asp-Asp-Ala-Ala-34FF-Val-S8-Phe-Trp-Gln-NH2 | 165 |
| I-268 | Ac-PL3-Asp-Ala-B5-Asp-Asp-Ala-Ala-Phe-Val-S8-hPhe-Trp-Gln-NH2 | 166 |
| I-269 | Ac-PL3-Asp-Ala-B5-Asp-Asp-Ala-Ala-Phe-Val-S8-hTyr-Trp-Gln-NH2 | 167 |
| I-270 | Ac-PL3-Asp-Ala-B5-Asp-2PyrA-Ala-Ala-Phe-Val-S8-Phe-Trp-Gln-NH2 | 168 |
| I-271 | Ac-PL3-Asp-Ala-B5-Asp-3PyrA-Ala-Ala-Phe-Val-S8-Phe-Trp-Gln-NH2 | 169 |
| I-272 | Ac-PL3-Asp-Ala-B5-Asp-4PyrA-Ala-Ala-Phe-Val-S8-Phe-Trp-Gln-NH2 | 170 |
| I-273 | Ac-PL3-Asp-Ala-B5-Asp-hTyr-Ala-Ala-Phe-Val-S8-Phe-Trp-Gln-NH2 | 171 |
| I-274 | Ac-PL3-Asp-Ala-B5-Asp-Tyr-Ala-Ala-Phe-Val-S8-Phe-Trp-Gln-NH2 | 172 |
| I-275 | Ac-PL3-Asp-Ala-B5-Asp-Asp-Ala-Ala-Phe-Val-S8-1NapA-Trp-Gln-NH2 | 173 |
| I-276 | Ac-PL3-Asp-Ala-B5-Asp-Asp-Ala-Ala-Phe-Val-S8-1NapA-Trp-Gln-NH2 | 173 |
| I-277 | Ac-PL3-Asp-Ala-B5-Asp-Asp-Ala-Ala-Phe-Val-S8-2NapA-Trp-Gln-NH2 | 174 |
| I-278 | Ac-PL3-Asp-Ala-B5-Asp-Asp-Ala-Ala-Phe-Val-S8-2NapA-Trp-Gln-NH2 | 174 |
| I-279 | Ac-PL3-Glu-Ala-B5-Asp-Asp-Ala-Ala-Phe-Val-S8-Phe-Trp-Gln-NH2 | 175 |
| I-280 | Ac-PL3-Glu-Ala-B5-Asp-Asp-Ala-Ala-Phe-Val-S8-Phe-Trp-Gln-NH2 | 175 |
| I-281 | Ac-PL3-Asp-Ala-B5-Glu-Asp-Ala-Ala-Phe-Val-S8-Phe-Trp-Gln-NH2 | 176 |
| I-282 | Ac-PL3-Asp-Ala-B5-Glu-Asp-Ala-Ala-Phe-Val-S8-Phe-Trp-Gln-NH2 | 176 |
| I-283 | Ac-PL3-Asp-Ala-B5-Asp-Asp-Ala-Ala-Phe-Val-S8-Asp-Trp-Gln-NH2 | 177 |
| I-284 | Ac-PL3-Asp-Ala-B5-Asp-Asp-Ala-Ala-Phe-Val-S8-Asn-Trp-Gln-NH2 | 178 |
| I-285 | Ac-PL3-Asp-Ala-B5-Asp-Asp-Ala-Ala-Phe-Val-S8-His-Trp-Gln-NH2 | 179 |
| I-286 | Ac-PL3-Asp-Ala-B5-Asp-Asp-Ala-Ala-Phe-Ala-S8-Phe-Trp-Ala-NH2 | 180 |
| I-287 | Ac-PL3-Asp-Ala-B5-Asp-Asp-Ala-Ala-Phe-Ala-S8-Phe-Trp-Gln-NH2 | 181 |
| I-288 | Ac-PL3-Asp-Ala-B5-Asp-Asp-Ala-Ala-Phe-Ala-S8-Phe-Trp-Gln-NH2 | 181 |
| I-289 | Ac-PL3-Asp-Ala-B5-Asp-Asp-Ala-Ala-Phe-Val-S8-Phe-Trp-NH2 | 182 |
| I-290 | Ac-PL3-Asp-Ala-B5-Asp-Asp-Ala-Ala-Phe-Val-S8-Phe-Trp-NH2 | 182 |
| I-291 | Ac-PL3-Asp-Ala-B5-Asp-Asp-Ala-Ala-Phe-Val-S8-Phe-Trp-Ala-NH2 | 183 |
| I-292 | Ac-PL3-Asp-Ala-B5-Asp-Asp-Ala-Ala-Phe-Val-S8-Phe-Trp-Ala-NH2 | 183 |
| I-293 | Ac-PL3-Asp-Ala-B5-Asp-TfeGA-Ala-Ala-Phe-Val-S8-Phe-BztA-Gln-NH2 | 184 |
| I-294 | Ac-PL3-Asp-Ala-B5-Asp-Asp-Ala-Ala-Phe-Gln-S8-Phe-BztA-Gln-NH2 | 185 |
| I-295 | Ac-PL3-Asp-Ala-B5-Asp-2NapA-Ala-Ala-Phe-Gln-S8-Phe-BztA-Gln-NH2 | 186 |
| I-296 | Ac-PL3-Asp-Ala-B5-Asp-2NapA-Ala-Ala-Phe-Gln-S8-Phe-BztA-Ala-NH2 | 187 |
| I-297 | Ac-PL3-Asp-Ala-B5-Asp-2NapA-Ala-Ala-Phe-Val-S8-Phe-BztA-Gln-NH2 | 188 |
| I-298 | Ac-PL3-Asp-Ala-B5-Asp-2NapA-Ala-Ala-Phe-Ser-S8-Phe-BztA-Gln-NH2 | 189 |
| I-299 | Ac-PL3-Asp-Hse-B5-Asp-Asp-Ala-Ala-Phe-Gln-S8-Phe-BztA-Ala-NH2 | 190 |
| I-300 | Ac-PL3-Asp-Ala-B5-Asp-Qui-Ala-Ala-Phe-Val-S8-Phe-BztA-Gln-NH2 | 191 |
| I-301 | Ac-PL3-Asp-Ala-B5-Asp-hPhe-Ala-Ala-Phe-Val-S8-Phe-BztA-Gln-NH2 | 192 |
| I-302 | Ac-PL3-Asp-Ala-B5-Asp-Asp-Ala-Ala-Phe-Val-S8-pff-BztA-Gln-NH2 | 193 |
| I-303 | Ac-PL3-Asp-Ala-B5-Asp-DipA-Ala-Ala-Phe-Val-S8-Phe-Trp-Gln-NH2 | 194 |
| I-304 | Ac-PL3-Asp-Ala-B5-Asp-His-Ala-Ala-Phe-Val-S8-Phe-Trp-Gln-NH2 | 195 |
| I-305 | Ac-PL3-Asp-Ala-B5-Asp-His-Ala-Ala-Phe-Val-S8-Phe-Trp-Gln-NH2 | 195 |
| I-306 | Ac-PL3-Asp-Ala-B5-Asp-Asp-Ala-Ala-3C1F-Val-S8-Phe-Trp-Gln-NH2 | 196 |
| I-307 | Ac-PL3-Asp-Ala-B5-Asp-Asp-Ala-Ala-3C1F-Val-S8-Phe-Trp-Gln-NH2 | 196 |
| I-308 | Ac-PL3-Asp-Ala-B5-Asp-Asp-Ala-Ala-4C1F-Val-S8-Phe-Trp-Gln-NH2 | 197 |
| I-309 | Ac-PL3-Asp-Ala-B5-Asp-Asp-Ala-Ala-4C1F-Val-S8-Phe-Trp-Gln-NH2 | 197 |
| I-310 | Ac-PL3-Asp-Ala-B5-Asp-Asp-Ala-Ala-Phe-Val-S8-3C1F-Trp-Gln-NH2 | 198 |
| I-311 | Ac-PL3-Asp-Ala-B5-Asp-Asp-Ala-Ala-Phe-Val-S8-3C1F-Trp-Gln-NH2 | 198 |
| I-312 | Ac-PL3-Asp-Ala-B5-Asp-Asp-Ala-Ala-Phe-Val-S8-4C1F-Trp-Gln-NH2 | 199 |
| I-313 | Ac-PL3-Asp-Ala-B5-Asp-Asp-Ala-Ala-Phe-Val-S8-4C1F-Trp-Gln-NH2 | 199 |
| I-314 | Ac-PL3-His-Ala-B5-Asp-Glu-Ala-Ala-Phe-Val-S8-Phe-Trp-Gln-NH2 | 200 |
| I-315 | Ac-PL3-His-Ala-B5-Asp-Glu-Ala-Ala-Phe-Val-S8-Phe-Trp-Gln-NH2 | 200 |
| I-316 | Ac-PL3-Asp-Ala-B5-Asp-Asp-Ala-Ala-Phe-Gln-S8-Phe-Trp-Ala-NH2 | 201 |
| I-317 | Ac-PL3-Asp-Ala-B5-Asp-Asp-Ala-Ala-2FF-Val-S8-Phe-Trp-Gln-NH2 | 202 |
| I-318 | Ac-PL3-Asp-Ala-B5-Asp-Asp-Ala-Ala-Phe-Val-S8-2FF-Trp-Gln-NH2 | 203 |
| I-319 | Ac-PL3-Asp-Ala-B5-Asp-Asp-Ala-Ala-Phe-Val-S8-2FF-Trp-Gln-NH2 | 203 |
| I-320 | Ac-PL3-Asp-Ala-B5-Asp-Asp-Ala-Ala-His-Val-S8-Phe-Trp-Gln-NH2 | 204 |
| I-321 | Ac-PL3-Asp-Ala-B5-Asp-Asp-Ala-Ala-His-Val-S8-Phe-Trp-Gln-NH2 | 204 |
| I-322 | Ac-PL3-Asp-Ala-B5-Asp-Asp-Ala-Ala-4AmPhe-Val-S8-Phe-Trp-Gln-NH2 | 205 |
| I-323 | Ac-PL3-Asp-Ala-B5-Asp-Asp-Ala-Ala-4AmPhe-Val-S8-Phe-Trp-Gln-NH2 | 205 |
| I-324 | Ac-PL3-Asp-Ala-B5-Asp-4AmPhe-Ala-Ala-Phe-Val-S8-Phe-Trp-Gln-NH2 | 206 |
| I-325 | Ac-PL3-Asp-Ala-B5-Asp-Asp-Ala-Ala-2Thi-Val-S8-Phe-Trp-Gln-NH2 | 207 |
| I-326 | Ac-PL3-Asp-Ala-B5-Asp-Asp-Ala-Ala-3Thi-Val-S8-Phe-Trp-Gln-NH2 | 208 |
| I-327 | Ac-PL3-Asp-Ala-B5-Asp-Asp-Ala-Ala-2FurA-Val-S8-Phe-Trp-Gln-NH2 | 209 |
| I-328 | Ac-PL3-Asp-Ala-B5-Asp-Asp-Ala-Ala-2FurA-Val-S8-Phe-Trp-Gln-NH2 | 209 |
| I-329 | Ac-PL3-Asp-Ala-B5-Asp-Asp-Ala-Ala-1meH-Val-S8-Phe-Trp-Gln-NH2 | 210 |
| I-330 | Ac-PL3-Asp-Ala-B5-Asp-Asp-Ala-Ala-1meH-Val-S8-Phe-Trp-Gln-NH2 | 210 |
| I-331 | Ac-PL3-Asp-Ala-B5-Asp-Asp-Ala-Ala-3meH-Val-S8-Phe-Trp-Gln-NH2 | 211 |
| I-332 | Ac-PL3-Asp-Ala-B5-Asp-Asp-Ala-Ala-3meH-Val-S8-Phe-Trp-Gln-NH2 | 211 |
| I-333 | Ac-PL3-Asp-Ala-B5-Asp-Asp-Ala-Ala-Phe-Ser-S8-Phe-WCHO-Ser-NH2 | 212 |
| I-334 | Ac-PL3-Asp-Ala-B5-Asp-Asp-Ala-Ala-Phe-Val-S8-Phe-WCHO-Ser-NH2 | 213 |
| I-335 | Ac-PL3-Asp-Ala-B5-Asp-Asp-Ala-Ala-Phe-Val-S8-Phe-WCHO-Ser-NH2 | 213 |
| I-336 | Ac-PL3-Asp-Ala-B5-Asp-Asp-Ala-Ala-Phe-Val-S8-2CBMF-BztA-Gln-NH2 | 214 |
| I-337 | Ac-PL3-Asp-Ala-B5-Asp-Asp-Ala-Ala-Phe-Val-S8-4CBMF-BztA-Gln-NH2 | 215 |
| I-338 | Ac-PL3-Asp-Ala-B5-Asp-Asp-Ala-Ala-Phe-Val-S8-4CBMF-BztA-Gln-NH2 | 215 |
| I-339 | Ac-PL3-Asp-Ala-B5-Asp-Asp-Ala-Ala-Phe-Val-S8-4COOHF-BztA-Gln-NH2 | 216 |
| I-340 | Ac-PL3-Asp-Ala-B5-Asp-Asp-Ala-Ala-Phe-Val-S8-4COOHF-BztA-Gln-NH2 | 216 |
| I-341 | Ac-PL3-Asp-Ala-B5-Asp-Asp-Ala-Ala-Phe-Val-S8-Tyr-BztA-Gln-NH2 | 217 |
| I-342 | Ac-PL3-Asp-Ala-B5-Asp-Asp-Ala-Ala-Phe-Thr-S8-Phe-BztA-Gln-NH2 | 218 |
| I-343 | Ac-PL3-Asp-Ala-B5-Asp-Asp-Ala-Ala-Phe-Asn-S8-Phe-BztA-Gln-NH2 | 219 |
| I-344 | Ac-PL3-Asp-Ala-B5-Asp-Asp-Ala-Ala-Phe-Asn-S8-Phe-BztA-Gln-NH2 | 219 |
| I-345 | Ac-PL3-Asp-S(OMe)-B5-Asp-Asp-Ala-Ala-Phe-Val-S8-Phe-BztA-Gln-NH2 | 220 |
| I-346 | Ac-PL3-Asp-Gln-B5-Asp-Asp-Ala-Ala-Phe-Val-S8-Phe-BztA-Gln-NH2 | 221 |
| I-347 | Ac-PL3-Asp-Gln-B5-Asp-Asp-Ala-Ala-Phe-Val-S8-Phe-BztA-Gln-NH2 | 221 |
| I-348 | Ac-PL3-Ala-Ala-B5-Asp-Asp-Ala-Ala-Phe-Val-S8-Phe-Trp-Gln-NH2 | 222 |
| I-349 | Ac-PL3-Ala-Ala-B5-Asp-Asp-Ala-Ala-Phe-Val-S8-Phe-Trp-Gln-NH2 | 222 |
| I-350 | Ac-PL3-Asp-Ala-B5-Ala-Asp-Ala-Ala-Phe-Val-S8-Phe-Trp-Gln-NH2 | 223 |
| I-351 | Ac-PL3-Asp-Ala-B5-Ala-Asp-Ala-Ala-Phe-Val-S8-Phe-Trp-Gln-NH2 | 223 |
| I-352 | Ac-PL3-Asp-Ala-B5-Asp-Ala-Ala-Ala-Phe-Val-S8-Phe-Trp-Gln-NH2 | 224 |
| I-353 | Ac-PL3-Asp-Ala-B5-Asp-Asp-Ala-Ala-Ala-Val-S8-Phe-Trp-Gln-NH2 | 225 |
| I-354 | Ac-PL3-Asp-Ala-B5-Asp-Asp-Ala-Ala-Ala-Val-S8-Phe-Trp-Gln-NH2 | 225 |
| I-355 | Ac-PL3-Asp-Ala-B5-Asp-Asp-Ala-Ala-Phe-Val-S8-Ala-Trp-Gln-NH2 | 226 |
| I-356 | Ac-PL3-Asp-Ala-B5-Asp-Asp-Ala-Ala-Phe-Val-S8-Ala-Trp-Gln-NH2 | 226 |
| I-357 | Ac-PL3-Asp-Ala-B5-Asp-Asp-Ala-Ala-Phe-Val-S8-Phe-Ala-Gln-NH2 | 227 |
| I-358 | Ac-PL3-Asp-Ala-B5-Asp-Asp-Ala-Ala-Phe-Val-S8-Phe-Ala-Gln-NH2 | 227 |
| I-359 | Ac-PL3-Asp-Ala-B5-Asp-Asp-Ala-Ala-Phe-Val-S8-4CNF-BztA-Gln-NH2 | 228 |
| I-360 | Ac-PL3-Asp-Ala-B5-Asp-Asp-Ala-Ala-Phe-Gln-S8-Phe-BztA-His-NH2 | 229 |
| I-361 | Ac-PL3-Asp-Ala-B5-Asp-Asp-Ala-Ala-Phe-Gln-S8-Phe-BztA-His-NH2 | 229 |
| I-362 | Ac-PL3-Asp-Ala-B5-Asp-Asp-Ala-Ala-Phe-Ala-S8-His-BztA-Gln-NH2 | 230 |
| I-363 | Ac-PL3-Asp-Ala-B5-Asp-Asp-Ala-Ala-His-Ala-S8-Phe-BztA-Gln-NH2 | 231 |
| I-364 | Ac-PL3-Asp-Ala-B5-Asp-Asp-Ala-Ala-His-Ala-S8-Phe-BztA-Gln-NH2 | 231 |
| I-365 | Ac-PL3-Asp-Ala-B5-Asp-Asp-Ala-Ala-2MeF-Ala-S8-Phe-BztA-Gln-NH2 | 232 |
| I-366 | Ac-PL3-Asp-Ala-B5-Asp-Asp-Ala-Ala-2MeF-Ala-S8-Phe-BztA-Gln-NH2 | 232 |
| I-367 | Ac-PL3-Asp-Ala-B5-Asp-Asp-Ala-Ala-Phe-Ala-S8-2MeF-BztA-Gln-NH2 | 233 |
| I-368 | Ac-PL3-Asp-Ala-B5-Asp-Asp-Ala-Ala-Phe-Val-S8-Phe-7F1NapA-Gln-NH2 | 234 |
| I-369 | Ac-PL3-Asp-Ala-B5-Asp-Asp-Ala-Ala-Phe-Ala-S8-4MeF-BztA-Gln-NH2 | 235 |
| I-370 | Ac-PL3-Asp-Ala-B5-Asp-Asp-Ala-Ala-2NapA-Ala-S8-Phe-BztA-Gln-NH2 | 236 |
| I-371 | Ac-PL3-Asp-Ala-B5-Asp-Asp-Ala-Ala-Phe-Ala-S8-3FF-BztA-Gln-NH2 | 237 |
| I-372 | Ac-PL3-Asp-Ala-B5-Asp-Glu-Ala-Ala-Phe-Ala-S8-3FF-BztA-Gln-NH2 | 238 |
| I-373 | Ac-PL3-Asp-Ala-B5-Asp-Asp-Ala-Ala-3F3MeF-Val-S8-Phe-BztA-Gln-NH2 | 239 |
| I-374 | Ac-PL3-Asp-Ala-B5-Asp-Asp-Ala-Ala-3F3MeF-Ala-S8-Phe-BztA-Gln-NH2 | 240 |
| I-375 | Ac-PL3-Asp-Ala-B5-Asp-Asp-Ala-Ala-3F3MeF-Ala-S8-Phe-BztA-Gln-NH2 | 240 |
| I-376 | Ac-PL3-Asp-Ala-B5-Asp-Asp-Ala-Ala-3F3MeF-Ala-S8-Phe-BztA-Gln-NH2 | 240 |
| I-377 | Ac-PL3-Asp-Ala-B5-Asp-Asp-Ala-Ala-3F3MeF-Ala-S8-Phe-BztA-Gln-NH2 | 240 |
| I-378 | Ac-PL3-Asp-Ala-B5-Asp-Asp-Ala-Ala-3F3MeF-Gln-S8-Phe-BztA-Gln-NH2 | 241 |
| I-379 | Ac-PL3-Asp-Ala-B5-Asp-Asp-Ala-Ala-3F3MeF-Gln-S8-Phe-BztA-Gln-NH2 | 241 |
| I-380 | Ac-PL3-Asp-Ala-B5-Asp-Asp-Ala-Ala-3F3MeF-Gln-S8-Phe-BztA-Gln-NH2 | 241 |
| I-381 | Ac-PL3-Asp-Ala-B5-Asp-Asn-Ala-Ala-Phe-Val-S8-3FF-BztA-Gln-NH2 | 242 |
| I-382 | Ac-PL3-Asp-Ala-B5-Asp-Asn-Ala-Ala-Phe-Val-S8-3FF-BztA-Gln-NH2 | 242 |
| I-383 | Ac-PL3-Asp-Ala-B5-Asp-Glu-Ala-Ala-Phe-Val-S8-2pyrA-BztA-Gln-NH2 | 243 |
| I-384 | Ac-PL3-Asp-Ala-B5-Asp-Glu-Ala-Ala-Phe-Val-S8-2pyrA-BztA-Gln-NH2 | 243 |
| I-385 | Ac-PL3-Asp-Ala-B5-Asp-Glu-Ala-Ala-Phe-Val-S8-4pyrA-BztA-Gln-NH2 | 244 |
| I-386 | Ac-PL3-Asp-Ala-B5-Asp-Glu-Ala-Ala-2pyrA-Val-S8-Phe-BztA-Gln-NH2 | 245 |
| I-387 | Ac-PL3-Asp-Ala-B5-Asp-Glu-Ala-Ala-2pyrA-Val-S8-Phe-BztA-Gln-NH2 | 245 |
| I-388 | Ac-PL3-Asp-Ala-B5-Asp-Glu-Ala-Ala-Phe-Val-S8-23FF-BztA-Gln-NH2 | 246 |
| I-389 | Ac-PL3-Asp-Ala-B5-Asp-Glu-Ala-Ala-Phe-Val-S8-23FF-BztA-Gln-NH2 | 246 |
| I-390 | Ac-PL3-Asp-Ala-B5-Asp-Glu-Ala-Ala-23FF-Val-S8-Phe-BztA-Gln-NH2 | 247 |
| I-391 | Ac-PL3-Asp-Ala-B5-Asp-Glu-Ala-Ala-23FF-Val-S8-Phe-BztA-Gln-NH2 | 247 |
| I-392 | Ac-PL3-Asp-Ala-B5-Asp-Glu-Ala-Ala-CypA-Val-S8-Phe-BztA-Gln-NH2 | 248 |
| I-393 | Ac-PL3-Asp-Ala-B5-Asp-Glu-Ala-Ala-Phe-Val-S8-CypA-BztA-Gln-NH2 | 249 |
| I-394 | Ac-PL3-Asp-Ala-B5-Asp-Glu-Ala-Ala-Phe-Val-S8-35FF-BztA-Gln-NH2 | 250 |
| I-395 | Ac-PL3-Asp-Ala-B5-Asp-Glu-Ala-Ala-Phe-Val-S8-3MeF-BztA-Gln-NH2 | 251 |
| I-396 | Ac-PL3-Asp-Ala-B5-Asp-His-Ala-Ala-Phe-Val-S8-3FF-BztA-Gln-NH2 | 252 |
| I-397 | Ac-PL3-Asp-Ala-B5-Asp-His-Ala-Ala-Phe-Val-S8-3FF-BztA-Gln-NH2 | 252 |
| I-398 | Ac-PL3-Asp-Ala-B5-Asp-His-Ala-Ala-Phe-Val-SeN-3FF-BztA-Gln-NH2 | 253 |
| I-399 | Ac-PL3-Asp-Ala-B5-Asp-His-Ala-Ala-Phe-Gln-S8-3FF-BztA-Gln-NH2 | 254 |
| I-400 | Ac-PL3-Asp-Ala-B5-Asp-His-Ala-Ala-Phe-Gln-SeN-3FF-BztA-Gln-NH2 | 255 |
| I-401 | Ac-PL3-Asp-Ala-B5-Asp-Glu-Ala-Ala-2Thi-Gln-S8-3FF-BztA-Gln-NH2 | 256 |
| I-402 | Ac-PL3-Asp-Ala-B5-Asp-2Thi-Ala-Ala-Phe-Val-S8-3FF-BztA-Gln-NH2 | 257 |
| I-403 | Ac-PL3-Asp-Ala-B5-Asp-1meH-Ala-Ala-Phe-Val-S8-3FF-BztA-Gln-NH2 | 258 |
| I-404 | Ac-PL3-Asp-Ala-B5-Asp-TfeGA-Ala-Ala-3MeF-Gln-S8-3FF-BztA-Gln-NH2 | 259 |
| I-405 | Ac-PL3-Asp-Ala-B5-Asp-TfeGA-Ala-Ala-3MeF-Gln-SeN-3FF-BztA-Gln-NH2 | 260 |
| I-406 | Ac-PL3-Asp-Ala-B5-Asp-Glu-Ala-Ala-Phe-Gln-S8-Phe-BztA-Gln-NH2 | 261 |
| I-407 | Ac-PL3-Asp-Ile-B5-Asp-Glu-Ala-Ala-Phe-Gln-S8-Phe-BztA-Gln-NH2 | 262 |
| I-408 | Ac-PL3-Asp-Ile-B5-Asp-Glu-Ala-Ala-Phe-Gln-S8-Phe-BztA-NH2 | 263 |
| I-409 | Ac-PL3-Asp-Ile-B5-Asp-Glu-Ala-Ala-Phe-Gln-S8-Phe-BztA-NH2 | 263 |
| I-410 | Ac-PL3-Asp-Val-B5-Asp-Glu-Ala-Ala-Phe-Gln-S8-Phe-BztA-Gln-NH2 | 264 |
| I-411 | Ac-PL3-Asp-nLeu-B5-Asp-Glu-Ala-Ala-Phe-Ala-S8-Phe-BztA-Gln-NH2 | 265 |
| I-412 | Ac-PL3-Asp-Ile-B5-Asp-Glu-Ala-Ala-2Thi-Gln-S8-3FF-BztA-Gln-NH2 | 266 |
| I-413 | Ac-PL3-Asp-Ala-B5-Asp-tetz-Ala-Ala-Phe-Gln-S8-Phe-BztA-Gln-NH2 | 267 |
| I-414 | Ac-PL3-Asp-Ala-B5-tetz-Glu-Ala-Ala-Phe-Gln-S8-Phe-BztA-Gln-NH2 | 268 |
| I-415 | Ac-PL3-Asp-Ala-B5-tetz-Glu-Ala-Ala-Phe-Gln-S8-Phe-BztA-Gln-NH2 | 268 |
| I-416 | Ac-PL3-tetz-Ala-B5-Asp-Glu-Ala-Ala-Phe-Gln-S8-Phe-BztA-Gln-NH2 | 269 |
| I-417 | Ac-PL3-tetz-Ala-B5-Asp-Glu-Ala-Ala-Phe-Gln-S8-Phe-BztA-Gln-NH2 | 269 |
| I-418 | Ac-PL3-Asp-Ala-B5-Asp-Glu-Ala-Ala-Phe-Gln-S8-Phe-BztA-His-NH2 | 270 |
| I-419 | Ac-PL3-Asp-Ala-B5-Asp-Glu-Ala-Ala-Phe-Gln-S8-Phe-BztA-His-NH2 | 270 |
| I-420 | Ac-PL3-Asp-Ala-B5-Asp-Glu-Ala-Ala-Phe-Thr-S8-Phe-BztA-His-NH2 | 271 |
| I-421 | Ac-PL3-Asp-Ala-B5-Asp-Glu-Ala-Ala-Phe-Asn-S8-Phe-BztA-His-NH2 | 272 |
| I-422 | Ac-PL3-Asp-Ala-B5-Asp-Glu-Ala-Ala-Phe-Asn-S8-Phe-BztA-His-NH2 | 272 |
| I-423 | Ac-PL3-Asp-Ala-B5-Asp-Glu-Ala-Ala-Phe-Thr-S8-Phe-BztA-Gln-NH2 | 273 |
| I-424 | Ac-PL3-Asp-Ala-B5-Asp-Glu-Ala-Ala-Phe-Asn-S8-Phe-BztA-Gln-NH2 | 274 |
| I-425 | Ac-PL3-Asp-Ala-B5-Asp-Glu-Ala-Ala-Phe-Asn-S8-Phe-BztA-Gln-NH2 | 274 |
| I-426 | Ac-PL3-Asp-Ala-B5-Asp-Glu-Ala-Ala-Phe-Gln-S8-3Thi-BztA-Gln-NH2 | 275 |
| I-427 | Ac-PL3-Asp-Ala-B5-Asp-Glu-Ala-Ala-Phe-Gln-S8-3Thi-BztA-Gln-NH2 | 275 |
| I-428 | Ac-PL3-Asp-Ala-B5-Asp-Glu-Ala-Ala-Phe-Gln-S8-2Thi-BztA-Gln-NH2 | 276 |
| I-429 | Ac-PL3-Asp-Ala-B5-Asp-Glu-Ala-Ala-3Thi-Gln-S8-3FF-BztA-Gln-NH2 | 277 |
| I-430 | Ac-PL3-Asp-Ala-B5-Asp-Glu-Ala-Ala-3Thi-Gln-S8-3FF-BztA-His-NH2 | 278 |
| I-431 | Ac-PL3-Asp-Ala-B5-Asp-Glu-Ala-Ala-3Thi-Gln-S8-3FF-BztA-NH2 | 279 |
| I-432 | Ac-PL3-Asp-Ala-B5-Asp-Glu-Ala-Ala-Phe-Asn-S8-Phe-BztA-NH2 | 280 |
| I-433 | Ac-PL3-Asp-Ala-B5-Asp-Glu-Ala-Ala-Phe-S(OMe)-S8-Phe-BztA-Gln-NH2 | 281 |
| I-434 | Ac-PL3-Asp-Ala-B5-Asp-Glu-Ala-Ala-Phe-Asn-S8-2FF-BztA-Gln-NH2 | 282 |
| I-435 | Ac-PL3-Asp-Ala-B5-Asp-Glu-Ala-Ala-Phe-Asn-S8-2C1F-BztA-Gln-NH2 | 283 |
| I-436 | Ac-PL3-Asp-Ala-B5-Asp-Glu-Ala-Ala-Phe-Asn-S8-2BrF-BztA-Gln-NH2 | 284 |
| I-437 | Ac-PL3-Asp-Ala-B5-Asp-Glu-Ala-Ala-Phe-Asn-S8-2OMeF-BztA-Gln-NH2 | 285 |
| I-438 | Ac-PL3-Asp-Ala-B5-Asp-Glu-Ala-Ala-Phe-Asn-S8-3meH-BztA-Gln-NH2 | 286 |
| I-439 | Ac-PL3-Asp-Ala-B5-Asp-Glu-Ala-Ala-Phe-Asn-S8-1meH-BztA-Gln-NH2 | 287 |
| I-440 | Ac-PL3-Asp-Ala-B5-Asp-Glu-Ala-Ala-Phe-Asn-S8-1meH-BztA-Gln-NH2 | 287 |
| I-441 | Ac-PL3-Asp-Ala-B5-Asp-Glu-Ala-Ala-Phe-Asn-S8-2NO2F-BztA-Gln-NH2 | 288 |
| I-442 | Ac-PL3-Asp-Ala-B5-Asp-Glu-Ala-Ala-Phe-Asn-S8-hPhe-BztA-Gln-NH2 | 289 |
| I-443 | Ac-PL3-Asp-Ala-B5-Asp-Glu-Ala-Ala-Phe-Asn-S8-hPhe-BztA-Gln-NH2 | 289 |
| I-444 | Ac-PL3-Asp-Ala-B5-Asp-Tetz-Ala-Ala-Phe-Asn-S8-Phe-BztA-Gln-NH2 | 290 |
| I-445 | Ac-PL3-Asp-Ala-B5-Tetz-Asp-Ala-Ala-Phe-Asn-S8-Phe-BztA-Gln-NH2 | 291 |
| I-446 | Ac-PL3-Asp-Ala-B5-Asp-Glu-Ala-Ala-2Thi-Asn-S8-Phe-BztA-Gln-NH2 | 292 |
| I-447 | Ac-PL3-Asp-Ala-B5-Asp-Glu-Ala-Ala-2Thi-Asn-S8-Phe-BztA-Gln-NH2 | 292 |
| I-448 | Ac-PL3-Asp-Ala-B5-Asp-Glu-Ala-Ala-3Thi-Asn-S8-Phe-BztA-Gln-NH2 | 293 |
| I-449 | Ac-PL3-Asp-Ala-B5-Asp-Glu-Ala-Ala-3Thi-Asn-S8-Phe-BztA-Gln-NH2 | 293 |
| I-450 | Ac-PL3-Asp-Ala-B5-Asp-Glu-Ala-Ala-2Thi-Gln-S8-3FF-Trp-Gln-NH2 | 294 |
| I-451 | Ac-PL3-Asp-Ala-B5-Asp-Glu-Ala-Ala-3Thi-Gln-S8-3FF-Trp-Gln-NH2 | 295 |
| I-452 | Ac-PL3-Asp-Ala-B5-Asp-Glu-Ala-Ala-3Thi-Gln-S8-3FF-Trp-Gln-NH2 | 295 |
| I-453 | Ac-PL3-Asp-Ala-B5-Asp-Glu-Ala-Ala-2FF-Asn-S8-Phe-BztA-Gln-NH2 | 296 |
| I-454 | Ac-PL3-Asp-Ala-B5-Asp-Glu-Ala-Ala-3FF-Asn-S8-Phe-BztA-Gln-NH2 | 297 |
| I-455 | Ac-PL3-Asp-Ala-B5-Asp-Glu-Ala-Ala-4FF-Asn-S8-Phe-BztA-Gln-NH2 | 298 |
| I-456 | Ac-PL3-Asp-Ala-B5-Asp-Glu-Ala-Ala-2MeF-Asn-S8-Phe-BztA-Gln-NH2 | 299 |
| I-457 | Ac-PL3-Asp-Ala-B5-Asp-Glu-Ala-Ala-2MeF-Asn-S8-Phe-BztA-Gln-NH2 | 299 |
| I-458 | Ac-PL3-Asp-Ala-B5-Asp-Glu-Ala-Ala-3MeF-Asn-S8-Phe-BztA-Gln-NH2 | 300 |
| I-459 | Ac-PL3-Asp-Ala-B5-Asp-Glu-Ala-Ala-4MeF-Asn-S8-Phe-BztA-Gln-NH2 | 301 |
| I-460 | Ac-PL3-Asp-Ala-B5-Asp-Glu-Ala-Ala-2FurA-Asn-S8-Phe-BztA-Gln-NH2 | 302 |
| I-461 | Ac-PL3-Asp-Ala-B5-Asp-Glu-Ala-Ala-4AmPhe-Asn-S8-Phe-BztA-Gln-NH2 | 303 |
| I-462 | Ac-PL3-Asp-Ala-B5-Asp-Glu-Ala-Ala-Phe-Asn-PyrS2-Phe-BztA-Gln-NH2 | 304 |
| I-463 | Ac-PL3-Asp-Ala-B5-Asp-Glu-Ala-Ala-Phe-Asn-PyrS3-Phe-BztA-Gln-NH2 | 305 |
| I-464 | Ac-PL3-Asp-Ala-B5-Asp-Glu-Ala-Ala-Phe-Asn-PyrS3-Phe-BztA-Gln-NH2 | 305 |
| I-465 | Ac-PL3-Asp-Ala-B5-Asp-Asp-Ala-Ala-Phe-Asn-S8-Phe-BztA-His-NH2 | 306 |
| I-466 | Ac-PL3-Asp-Ala-B5-Asp-Asp-Ala-Ala-Phe-Asn-S8-Phe-BztA-His-NH2 | 306 |
| I-467 | Ac-PL3-Asp-Ile-B5-Asp-Asp-Ala-Ala-Phe-Asn-S8-Phe-BztA-Gln-NH2 | 307 |
| I-468 | Ac-PL3-Asp-Ile-B5-Asp-Asp-Ala-Ala-Phe-Asn-S8-Phe-BztA-Gln-NH2 | 307 |
| I-469 | Ac-PL3-Asp-Ile-B5-Asp-Asp-Ala-Ala-Phe-Asn-S8-Phe-BztA-His-NH2 | 308 |
| I-470 | Ac-PL3-Asp-Ile-B5-Asp-Asp-Ala-Ala-Phe-Asn-S8-Phe-BztA-His-NH2 | 308 |
| I-471 | Ac-PL3-Asp-Ile-B5-Asp-Glu-Ala-Ala-Phe-Gln-S8-Phe-BztA-His-NH2 | 309 |
| I-472 | Ac-PL3-Asp-Ile-B5-Asp-Glu-Ala-Ala-Phe-Asn-S8-Phe-BztA-His-NH2 | 310 |
| I-473 | Ac-PL3-Asp-Abu-B5-Asp-Glu-Ala-Ala-Phe-Asn-S8-Phe-BztA-Gln-NH2 | 311 |
| I-474 | Ac-PL3-Asp-Abu-B5-Asp-Glu-Ala-Ala-Phe-Asn-S8-Phe-BztA-Gln-NH2 | 311 |
| I-475 | Ac-PL3-Asp-aIle-B5-Asp-Glu-Ala-Ala-Phe-Asn-S8-Phe-BztA-Gln-NH2 | 312 |
| I-476 | Ac-PL3-Asp-aIle-B5-Asp-Glu-Ala-Ala-Phe-Asn-S8-Phe-BztA-Gln-NH2 | 312 |
| I-477 | Ac-PL3-Asp-Thr-B5-Asp-Glu-Ala-Ala-Phe-Gln-S8-Phe-BztA-Gln-NH2 | 313 |
| I-478 | Ac-PL3-Asp-Ala-B5-Asp-Glu-Ala-Ala-Phe-Asn-S8-Phe-BztA-1MeH-NH2 | 314 |
| I-479 | Ac-PL3-Asp-Ala-B5-Asp-Glu-Ala-Ala-Phe-Asn-S8-Phe-BztA-1MeH-NH2 | 314 |
| I-480 | Ac-PL3-Asp-Ala-B5-Asp-Glu-Ala-Ala-Phe-Asn-S8-Phe-BztA-3MeH-NH2 | 315 |
| I-481 | Ac-PL3-Asp-Ala-B5-Asp-Glu-Ala-Ala-Phe-Asn-S8-Phe-BztA-3MeH-NH2 | 315 |
| I-482 | Ac-PL3-Asp-Ile-B5-Asp-Asp-Ala-Ala-Phe-Gln-S8-Phe-BztA-Dab-NH2 | 316 |
| I-483 | Ac-PL3-Asp-Ile-B5-Asp-Asp-Ala-Ala-Phe-Gln-S8-[Acryl]Dap-BztA-Gln-NH2 | 317 |
| I-484 | Ac-PL3-Asp-Ile-B5-Asp-Asp-Ala-Ala-Phe-Gln-S8-[Acryl]Dap-BztA-Gln-NH2 | 317 |
| I-485 | Ac-PL3-Asp-Ile-B5-Asp-Glu-Ala-Ala-2Thi-Gln-S8-Phe-BztA-Gln-NH2 | 318 |
| I-486 | Ac-PL3-Asp-Ala-B5-Asp-Glu-Ala-Ala-2Thi-Gln-S8-Phe-BztA-Gln-NH2 | 319 |
| I-487 | Ac-PL3-Asp-Ala-B5-Asp-Glu-Ala-Ala-2Thi-Gln-S8-Phe-BztA-Gln-NH2 | 319 |
| I-488 | Ac-PL3-Asp-Ile-B5-Asp-Glu-Ala-Ala-2Thi-Gln-S8-Phe-BztA-His-NH2 | 320 |
| I-489 | Ac-PL3-Asp-Ile-B5-Asp-Asp-Ala-Ala-2Thi-Gln-S8-Phe-BztA-His-NH2 | 321 |
| I-490 | Ac-PL3-Asp-Ala-B5-Asp-Glu-Ala-Ala-Phe-Gln-S8-Phe-BztA-2FurA-NH2 | 322 |
| I-491 | Ac-PL3-Asp-Ala-B5-Asp-Glu-Ala-Ala-Phe-Gln-S8-Phe-BztA-Hse-NH2 | 323 |
| I-492 | Ac-PL3-Asp-Ala-B5-Asp-Glu-Ala-Ala-Phe-Gln-S8-Phe-BztA-Hse-NH2 | 323 |
| I-493 | Ac-PL3-Asp-Ala-B5-Asp-Glu-Ala-Ala-Phe-Gln-S8-Phe-BztA-DSer-NH2 | 324 |
| I-494 | Ac-PL3-Asp-Ala-B5-Asp-Glu-Ala-Ala-Phe-Gln-S8-Phe-BztA-DGIn-NH2 | 325 |
| I-495 | Ac-PL3-Asp-Ile-B5-Asp-Glu-Ala-Ala-Phe-Gln-S8-Phe-BztA-Asn-NH2 | 326 |
| I-496 | Ac-PL3-Asp-Ala-B5-Hse-Glu-Ala-Ala-Phe-Gln-S8-Phe-BztA-Gln-NH2 | 327 |
| I-497 | Ac-PL3-Asp-Ala-B5-Hse-Glu-Ala-Ala-Phe-Gln-S8-Phe-BztA-Gln-NH2 | 327 |
| I-498 | Ac-PL3-Asp-Ala-B5-Hse-Glu-Ala-Ala-Phe-Gln-S8-Phe-BztA-Gln-NH2 | 327 |
| I-499 | Ac-PL3-Asp-Ala-B5-Asp-TfeGA-Ala-Ala-Phe-Gln-S8-Phe-BztA-His-NH2 | 328 |
| I-500 | Ac-PL3-Asp-Ile-B5-Asp-TfeGA-Ala-Ala-Phe-Gln-S8-Phe-BztA-His-NH2 | 329 |
| I-501 | Ac-PL3-Asp-F3CA-B5-Asp-Glu-Ala-Ala-Phe-Gln-S8-Phe-BztA-Gln-NH2 | 330 |
| I-502 | Ac-PL3-Asp-F3CA-B5-Asp-Glu-Ala-Ala-Phe-Gln-S8-Phe-BztA-Gln-NH2 | 330 |
| I-503 | Ac-PL3-Asp-HF2CA-B5-Asp-Glu-Ala-Ala-Phe-Gln-S8-Phe-BztA-Gln-NH2 | 331 |
| I-504 | Ac-PL3-Asp-F3CA-B5-Asp-Glu-Ala-Ala-Phe-Gln-S8-Phe-BztA-His-NH2 | 332 |
| I-505 | Ac-PL3-Asp-Leu-B5-Asp-Glu-Ala-Ala-Phe-Gln-S8-Phe-BztA-Gln-NH2 | 333 |
| I-506 | Ac-PL3-Asp-Leu-B5-Asp-Glu-Ala-Ala-Phe-Gln-S8-Phe-BztA-His-NH2 | 334 |
| I-507 | Ac-PL3-Asp-Ala-B5-Asp-Glu-Ala-Ala-Phe-Gln-S8-Phe-BztA-Thr-NH2 | 335 |
| I-508 | Ac-PL3-Asp-Ala-B5-Asp-Glu-Ala-Ala-Phe-Gln-S8-Phe-BztA-Thr-NH2 | 335 |
| I-509 | Ac-PL3-Asp-Ala-B5-Asp-Glu-Ala-Ala-Phe-Gln-S8-Phe-BztA-Abu-NH2 | 336 |
| I-510 | Ac-PL3-Asp-Ala-B5-Asp-Glu-Ala-Ala-Phe-Asn-S8-2OMeF-BztA-His-NH2 | 337 |
| I-511 | Ac-PL3-Asp-Ala-B5-Asp-Glu-Ala-Ala-Phe-Asn-S8-2OMeF-BztA-His-NH2 | 337 |
| I-512 | Ac-PL3-Asp-Ile-B5-Asp-Glu-Ala-Ala-Phe-Asn-S8-2OMeF-BztA-His-NH2 | 338 |
| I-513 | Ac-PL3-Asp-Ala-B5-Asp-Glu-Ala-Ala-Phe-Asn-S8-2CBMF-BztA-His-NH2 | 339 |
| I-514 | Ac-PL3-Asp-Ile-B5-Asp-Glu-Ala-Ala-Phe-Asn-S8-2CBMF-BztA-His-NH2 | 340 |
| I-515 | Ac-PL3-Asp-Ala-B5-Asp-Glu-Ala-Ala-Phe-Asn-S8-3Thi-BztA-His-NH2 | 341 |
| I-516 | Ac-PL3-Asp-Ala-B5-Asp-Glu-Ala-Ala-Phe-Asn-S8-3Thi-BztA-His-NH2 | 341 |
| I-517 | Ac-PL3-Asp-Ile-B5-Asp-Glu-Ala-Ala-Phe-Asn-S8-3Thi-BztA-His-NH2 | 342 |
| I-518 | Ac-PL3-Asp-Ala-B5-Asp-Glu-Ala-Ala-Phe-Asn-S8-3Thi-BztA-Gln-NH2 | 343 |
| I-519 | Ac-PL3-Asp-Ala-B5-Asp-Glu-Ala-Ala-Phe-Asn-S8-3Thi-BztA-Gln-NH2 | 343 |
| I-520 | Ac-PL3-Asp-Ile-B5-Asp-Glu-Ala-Ala-Phe-Asn-S8-3Thi-BztA-Gln-NH2 | 344 |
| I-521 | Ac-PL3-Asp-Ile-B5-Asp-Glu-Ala-Ala-Phe-Asn-S8-3Thi-BztA-Gln-NH2 | 344 |
| I-522 | Ac-PL3-Asp-Nva-B5-Asp-Glu-Ala-Ala-Phe-Gln-S8-Phe-BztA-Gln-NH2 | 345 |
| I-523 | Ac-PL3-Asp-Nva-B5-Asp-Glu-Ala-Ala-Phe-Gln-S8-Phe-BztA-Gln-NH2 | 345 |
| I-524 | Ac-PL3-Asp-Ala-B5-Asp-Glu-Ala-Ala-4Thz-Asn-S8-Phe-BztA-Gln-NH2 | 346 |
| I-525 | Ac-PL3-Asp-Ala-B5-Asp-Glu-Ala-Ala-Phe-Gln-S8-2OMeF-BztA-Gln-NH2 | 347 |
| I-526 | Ac-PL3-Asp-Ala-B5-Asp-Glu-Ala-Ala-Phe-Gln-S8-2OMeF-BztA-Gln-NH2 | 347 |
| I-527 | Ac-PL3-Asp-Ile-B5-Asp-Glu-Ala-Ala-Phe-Gln-S8-2OMeF-BztA-Gln-NH2 | 348 |
| I-528 | Ac-PL3-Asp-Ile-B5-Asp-Glu-Ala-Ala-Phe-Gln-S8-2OMeF-BztA-Gln-NH2 | 348 |
| I-529 | Ac-PL3-Asp-Ala-B5-[MeSO2]Dap-Glu-Ala-Ala-Phe-Asn-S8-Phe-BztA-Gln-NH2 | 349 |
| I-530 | Ac-PL3-Asp-Ala-B5-[MeSO2]Dap-Glu-Ala-Ala-Phe-Asn-S8-Phe-BztA-Gln-NH2 | 349 |
| I-531 | Ac-PL3-Asp-Ala-B5-[MeSO2]Dab-Glu-Ala-Ala-Phe-Asn-S8-Phe-BztA-Gln-NH2 | 350 |
| I-532 | Ac-PL3-Asp-Ala-B5-[MeSO2]Dab-Glu-Ala-Ala-Phe-Asn-S8-Phe-BztA-Gln-NH2 | 350 |
| I-533 | Ac-PL3-Asp-Ala-B5-Asp-[MeSO2]Dap-Ala-Ala-Phe-Asn-S8-Phe-BztA-Gln-NH2 | 351 |
| I-534 | Ac-PL3-[MeSO2]Dap-Ala-B5-Asp-Glu-Ala-Ala-Phe-Asn-S8-Phe-BztA-Gln-NH2 | 352 |
| I-535 | Ac-PL3-Asp-Ala-B5-[NHiPr]AsnR-Asp-Ala-Ala-Phe-Val-S8-Phe-BztA-Gln-NH2 | 353 |
| I-536 | Ac-PL3-Asp-Ala-B5-[NHEt]AsnR-Asp-Ala-Ala-Phe-Val-S8-Phe-BztA-Gln-NH2 | 354 |
| I-537 | Ac-PL3-Asp-Ala-B5-[NHnPr]AsnR-Asp-Ala-Ala-Phe-Val-S8-Phe-BztA-Gln-NH2 | 355 |
| I-538 | Ac-PL3-Asp-Ala-B5-[NHCyPr]AsnR-Asp-Ala-Ala-Phe-Val-S8-Phe-BztA-Gln-NH2 | 356 |
| I-539 | Ac-PL3-Asp-Ala-B5-[NHCyBu]AsnR-Asp-Ala-Ala-Phe-Val-S8-Phe-BztA-Gln-NH2 | 357 |
| I-540 | Ac-PL3-Asp-Ala-B5-[NHMe]AsnR-Asp-Ala-Ala-Phe-Val-S8-Phe-BztA-Gln-NH2 | 358 |
| I-541 | Ac-PL3-Asp-TOMe-B5-Asp-Glu-Ala-Ala-Phe-Gln-S8-Phe-BztA-Gln-NH2 | 359 |
| I-542 | Ac-PL3-Asp-TOMe-B5-Asp-Glu-Ala-Ala-Phe-Gln-S8-Phe-BztA-Gln-NH2 | 359 |
| I-543 | Ac-PL3-Asp-Leu-B5-Asp-Glu-Ala-Ala-Phe-Asn-S8-Phe-BztA-His-NH2 | 360 |
| I-544 | Ac-PL3-Asp-Npg-B5-Asp-Glu-Ala-Ala-Phe-Gln-S8-Phe-BztA-Gln-NH2 | 361 |
| I-545 | Ac-PL3-Asp-hLeu-B5-Asp-Glu-Ala-Ala-Phe-Gln-S8-Phe-BztA-Gln-NH2 | 362 |
| I-546 | Ac-PL3-Asp-Npg-B5-Asp-Asp-Ala-Ala-Phe-Asn-S8-Phe-BztA-Gln-NH2 | 363 |
| I-547 | Ac-PL3-Asp-hLeu-B5-Asp-Asp-Ala-Ala-Phe-Asn-S8-Phe-BztA-Gln-NH2 | 364 |
| I-548 | Ac-PL3-Asp-hLeu-B5-Asp-Asp-Ala-Ala-Phe-Asn-S8-Phe-BztA-Gln-NH2 | 364 |
| I-549 | Ac-PL3-Asp-Ile-B5-Asp-Asp-Ala-Ala-Phe-Asn-S8-2OMeF-BztA-Gln-NH2 | 365 |
| I-550 | Ac-PL3-Asp-Ile-B5-Asp-Asp-Ala-Ala-Phe-Asn-S8-2OMeF-BztA-Gln-NH2 | 365 |
| I-551 | Ac-PL3-Asp-Ile-B5-Asp-Asp-Ala-Ala-Phe-Asn-S8-3Thi-BztA-Gln-NH2 | 366 |
| I-552 | Ac-PL3-Asp-Ile-B5-Asp-Asp-Ala-Ala-Phe-Asn-S8-3Thi-BztA-Gln-NH2 | 366 |
| I-553 | Ac-PL3-Asp-Ile-B5-Asp-Asp-Ala-Ala-Phe-Asn-S8-2OMeF-BztA-His-NH2 | 367 |
| I-554 | Ac-PL3-Asp-Ile-B5-Asp-Asp-Ala-Ala-Phe-Asn-S8-2OMeF-BztA-His-NH2 | 367 |
| I-555 | Ac-PL3-Asp-Ile-B5-Asp-Asp-Ala-Ala-Phe-Asn-S8-3Thi-BztA-His-NH2 | 368 |
| I-556 | Ac-PL3-Asp-Ile-B5-Asp-Asp-Ala-Ala-Phe-Asn-S8-3Thi-BztA-His-NH2 | 368 |
| I-557 | Ac-PL3-Asp-Ile-B5-Asp-Glu-Ala-Ala-Phe-Asn-S8-Phe-BztA-Gln-NH2 | 369 |
| I-558 | Ac-PL3-Asp-Leu-B5-Asp-Asp-Ala-Ala-Phe-Gln-S8-Phe-BztA-Gln-NH2 | 370 |
| I-559 | Ac-PL3-Asp-Leu-B5-Asp-Asp-Ala-Ala-Phe-Gln-S8-Phe-BztA-Gln-NH2 | 370 |
| I-560 | Ac-PL3-Asp-Leu-B5-Asp-Asp-Ala-Ala-Phe-Gln-S8-Phe-BztA-His-NH2 | 371 |
| I-561 | Ac-PL3-Asp-Leu-B5-Asp-Asp-Ala-Ala-Phe-Gln-S8-Phe-BztA-His-NH2 | 371 |
| I-562 | Ac-PL3-Asp-Leu-B5-Asp-Asp-Ala-Ala-Phe-Asn-S8-Phe-BztA-Gln-NH2 | 372 |
| I-563 | Ac-PL3-Asp-Leu-B5-Asp-Asp-Ala-Ala-Phe-Asn-S8-Phe-BztA-Gln-NH2 | 372 |
| I-564 | Ac-PL3-Asp-Leu-B5-Asp-Asp-Ala-Ala-Phe-Asn-S8-Phe-BztA-His-NH2 | 373 |
| I-565 | Ac-PL3-Asp-Leu-B5-Asp-Asp-Ala-Ala-Phe-Asn-S8-Phe-BztA-His-NH2 | 373 |
| I-566 | Ac-PL3-Asp-Val-B5-Asp-Asp-Ala-Ala-Phe-Asn-S8-Phe-BztA-Gln-NH2 | 374 |
| I-567 | Ac-PL3-Asp-Val-B5-Asp-Asp-Ala-Ala-Phe-Asn-S8-Phe-BztA-Gln-NH2 | 374 |
| I-568 | Ac-PL3-Asp-Abu-B5-Asp-Glu-Ala-Ala-Phe-Gln-S8-Phe-BztA-Gln-NH2 | 375 |
| I-569 | Ac-PL3-Asp-Abu-B5-Asp-Glu-Ala-Ala-Phe-Gln-S8-Phe-BztA-Gln-NH2 | 375 |
| I-570 | Ac-PL3-Asp-Abu-B5-Asp-Glu-Ala-Ala-Phe-Gln-S8-Phe-BztA-His-NH2 | 376 |
| I-571 | Ac-PL3-Asp-Abu-B5-Asp-Glu-Ala-Ala-Phe-Gln-S8-Phe-BztA-His-NH2 | 376 |
| I-572 | Ac-PL3-Asp-Abu-B5-Asp-Asp-Ala-Ala-Phe-Asn-S8-Phe-BztA-Gln-NH2 | 377 |
| I-573 | Ac-PL3-Asp-Abu-B5-Asp-Asp-Ala-Ala-Phe-Asn-S8-Phe-BztA-Gln-NH2 | 377 |
| I-574 | Ac-PL3-Asp-Abu-B5-Asp-Asp-Ala-Ala-Phe-Asn-S8-Phe-BztA-His-NH2 | 378 |
| I-575 | Ac-PL3-Asp-Abu-B5-Asp-Asp-Ala-Ala-Phe-Asn-S8-Phe-BztA-His-NH2 | 378 |
| I-576 | Ac-PL3-Asp-Leu-B5-Asp-Glu-Ala-Ala-Phe-Asn-S8-Phe-BztA-Gln-NH2 | 379 |
| I-577 | Ac-PL3-Asp-Leu-B5-Asp-Glu-Ala-Ala-Phe-Asn-S8-Phe-BztA-Gln-NH2 | 379 |
| I-578 | Ac-PL3-Asp-Ala-B5-Asp-Asp-Ala-Ala-Phe-Gln-S8-Phe-BztA-Gln-NH2 | 185 |
| I-579 | Ac-Phe-Asp-Ala-R8-Asp-Asp-Ala-Ala-Phe-Gln-PyrS-Phe-Trp-Gln-NH2 | 380 |
| I-580 | Ac-PL3-Asp-Ile-B5-Asp-Asp-Ala-Ala-Phe-Gln-S8-Phe-BztA-His-NH2 | 381 |
| I-581 | Ac-PL3-Asp-Ile-B5-Asp-Asp-Ala-Ala-Phe-Gln-S8-Phe-BztA-His-NH2 | 381 |
| I-582 | Ac-PL3-Asp-Npg-B5-Asp-Asp-Ala-Ala-Phe-Asn-S8-Phe-BztA-His-NH2 | 382 |
| I-583 | Ac-PL3-Asp-hLeu-B5-Asp-Asp-Ala-Ala-Phe-Asn-S8-Phe-BztA-His-NH2 | 383 |
| I-584 | Ac-PL3-Asp-hLeu-B5-Asp-Asp-Ala-Ala-Phe-Asn-S8-Phe-BztA-His-NH2 | 383 |
| I-585 | Ac-PL3-Asp-Ile-B5-Asp-Glu-Ala-Ala-BztA-Gln-S8-Phe-Phe-Gln-NH2 | 384 |
| I-586 | Ac-PL3-Asp-Ile-B5-Asp-Glu-Ala-Ala-BztA-Gln-S8-Phe-Phe-Gln-NH2 | 384 |
| I-587 | Ac-PL3-Asp-Ile-B5-Phe-Glu-Ala-Ala-Phe-Gln-S8-Asp-BztA-Gln-NH2 | 385 |
| I-588 | Ac-PL3-Phe-Ile-B5-Asp-Glu-Ala-Ala-Asp-Gln-S8-Phe-BztA-Gln-NH2 | 386 |
| I-589 | Ac-PL3-Asp-[AzAc]Lys-B5-Asp-Glu-Ala-Ala-Phe-Gln-S8-Phe-BztA-Gln-NH2 | 387 |
| I-590 | Ac-PL3-Asp-[AzAc]Lys-B5-Asp-Glu-Ala-Ala-Phe-Gln-S8-Phe-BztA-Gln-NH2 | 387 |
| I-591 | Ac-PL3-Asp-Ile-B5-Asp-Glu-[AzAc]Lys-Ala-Phe-Gln-S8-Phe-BztA-Gln-NH2 | 388 |
| I-592 | Ac-PL3-Asp-Ile-B5-Asp-Glu-[AzAc]Lys-Ala-Phe-Gln-S8-Phe-BztA-Gln-NH2 | 388 |
| I-593 | Ac-PL3-Asp-Ile-B5-Asp-Glu-Ala-[AzAc]Lys-Phe-Gln-S8-Phe-BztA-Gln-NH2 | 389 |
| I-594 | Ac-PL3-Asp-Ile-B5-Asp-Glu-Ala-Ala-Phe-[AzAc]Lys-S8-Phe-BztA-Gln-NH2 | 390 |
| I-595 | Ac-PL3-Asp-Ile-B5-Asp-Glu-Ala-Ala-Phe-Gln-S8-Phe-BztA-[AzAc]Lys-NH2 | 391 |
| I-596 | Ac-PL3-Asp-Ile-B5-Asp-Glu-Ala-Ala-Phe-Gln-S8-Phe-BztA-Gln-[AzAc]Lys-NH2 | 392 |
| I-597 | Ac-PL3-Asp-Ile-B5-Asp-Glu-Ala-Ala-Phe-Gln-S8-Phe-BztA-Gln-[AzAc]Lys-NH2 | 392 |
| I-598 | Nic-PL3-Asp-Ile-B5-Asp-Glu-Ala-Ala-Phe-Gln-S8-Phe-BztA-Gln-NH2 | 393 |
| I-599 | Pic-PL3-Asp-Ile-B5-Asp-Glu-Ala-Ala-Phe-Gln-S8-Phe-BztA-Gln-NH2 | 394 |
| I-600 | Bnc-PL3-Asp-Ile-B5-Asp-Glu-Ala-Ala-Phe-Gln-S8-Phe-BztA-Gln-NH2 | 395 |
| I-601 | Bnc-PL3-Asp-Ile-B5-Asp-Glu-Ala-Ala-Phe-Gln-S8-Phe-BztA-Gln-NH2 | 395 |
| I-602 | 2PyPrpc-PL3-Asp-Ile-B5-Asp-Glu-Ala-Ala-Phe-Gln-S8-Phe-BztA-Gln-NH2 | 396 |
| I-603 | Ac-PL3-Asp-Ile-B5-Asp-Gln-Ala-Ala-Phe-Gln-S8-Phe-BztA-Gln-NH2 | 397 |
| I-604 | Ac-PL3-Asp-Ile-B5-Asp-Cit-Ala-Ala-Phe-Gln-S8-Phe-BztA-Gln-NH2 | 398 |
| I-605 | Ac-PL3-Asp-Ile-B5-Asp-Cit-Ala-Ala-Phe-Gln-S8-Phe-BztA-Gln-NH2 | 398 |
| I-606 | Ac-PL3-Asp-Ile-B5-Asp-4COOHF-Ala-Ala-Phe-Gln-S8-Phe-BztA-Gln-NH2 | 399 |
| I-607 | Ac-PL3-Asp-Ile-B5-Asp-4COOHF-Ala-Ala-Phe-Gln-S8-Phe-BztA-Gln-NH2 | 399 |
| I-608 | Ac-PL3-Asp-Ile-B5-Asp-EtGa-Ala-Ala-Phe-Gln-S8-Phe-BztA-Gln-NH2 | 400 |
| I-609 | Ac-PL3-Asp-Ile-B5-Asp-EtGa-Ala-Ala-Phe-Gln-S8-Phe-BztA-Gln-NH2 | 400 |
| I-610 | Ac-PL3-Asp-Ile-B5-Asp-TfeGA-Ala-Ala-Phe-Gln-S8-Phe-BztA-Gln-NH2 | 401 |
| I-611 | Ac-PL3-Asp-Ile-B5-Asp-TfeGA-Ala-Ala-Phe-Gln-S8-Phe-BztA-Gln-NH2 | 401 |
| I-612 | Ac-PL3-Asp-Ile-B5-Asp-Aad-Ala-Ala-Phe-Gln-S8-Phe-BztA-Gln-NH2 | 402 |
| I-613 | Ac-PL3-Asp-Ile-B5-Asp-Arg-Ala-Ala-Phe-Gln-S8-Phe-BztA-Gln-NH2 | 403 |
| I-614 | Ac-PL3-Asp-Ile-B5-Asp-Arg-Ala-Ala-Phe-Gln-S8-Phe-BztA-Gln-NH2 | 403 |
| I-615 | Ac-PL3-Asp-Cha-B5-Asp-Glu-Ala-Ala-Phe-Asn-S8-Phe-BztA-His-NH2 | 404 |
| I-616 | Ac-PL3-Asp-Leu-B5-Asp-Glu-Ala-Ala-Phe-Cit-S8-Phe-BztA-His-NH2 | 405 |
| I-617 | Ac-PL3-Asp-Leu-B5-Asp-Glu-Ala-Ala-Phe-Thr-S8-Phe-BztA-His-NH2 | 406 |
| I-618 | Ac-PL3-Asp-Leu-B5-Asp-Glu-Ala-Ala-Phe-Thr-S8-Phe-BztA-His-NH2 | 406 |
| I-619 | Ac-PL3-Asp-Cha-B5-Asp-Glu-Ala-Ala-Phe-Asn-S8-Phe-BztA-Gln-NH2 | 407 |
| I-620 | Ac-PL3-Asp-Cha-B5-Asp-Glu-Ala-Ala-Phe-Asn-S8-Phe-BztA-Gln-NH2 | 407 |
| I-621 | Ac-PL3-Asp-Leu-B5-Asp-Glu-Ala-Ala-Phe-Cit-S8-Phe-BztA-Gln-NH2 | 408 |
| I-622 | Ac-PL3-Asp-Leu-B5-Asp-Glu-Ala-Ala-Phe-Thr-S8-Phe-BztA-Gln-NH2 | 409 |
| I-623 | Ac-PL3-Asp-Leu-B5-Asp-Glu-Ala-Ala-Phe-Thr-S8-Phe-BztA-Gln-NH2 | 409 |
| I-624 | Ac-PL3-Asp-Leu-B5-Asp-Glu-Ala-Ala-Phe-Asn-S8-Phe-BztA-NH2 | 410 |
| I-625 | Ac-PL3-Asp-Leu-B5-Asp-Glu-Ala-Ala-Phe-Asn-S8-Phe-BztA-NH2 | 410 |
| I-626 | Ac-PL3-Asp-Cha-B5-Asp-Glu-Ala-Ala-Phe-Asn-S8-Phe-BztA-NH2 | 411 |
| I-627 | Ac-PL3-Asp-Cha-B5-Asp-Glu-Ala-Ala-Phe-Asn-S8-Phe-BztA-NH2 | 411 |
| I-628 | Ac-PL3-Asp-Leu-B5-Asp-Glu-Ala-Ala-Phe-Cit-S8-Phe-BztA-NH2 | 412 |
| I-629 | Ac-PL3-Asp-Leu-B5-Asp-Glu-Ala-Ala-Phe-Cit-S8-Phe-BztA-NH2 | 412 |
| I-630 | Ac-PL3-Asp-Ile-B5-[Tf]Dap-Glu-Ala-Ala-Phe-Gln-S8-Phe-BztA-Gln-NH2 | 413 |
| I-631 | Ac-PL3-Asp-Ile-B5-[Tf]Dap-Glu-Ala-Ala-Phe-Gln-S8-Phe-BztA-Gln-NH2 | 413 |
| I-632 | Ac-PL3-[Tf]Dap-Ile-B5-Asp-Glu-Ala-Ala-Phe-Gln-S8-Phe-BztA-Gln-NH2 | 414 |
| I-633 | Ac-PL3-Asp-Ile-B5-Asp-Glu-Ala-Ala-Phe-Gln-S8-Phe-BztA-Arg-NH2 | 415 |
| I-634 | Ac-PL3-Asp-Ile-B5-Asp-Glu-Ala-Ala-Phe-Gln-S8-Phe-BztA-Leu-NH2 | 416 |
| I-635 | Ac-PL3-Asp-Ile-B5-Asp-Glu-Ala-Ala-Phe-Gln-S8-Phe-BztA-Phe-NH2 | 417 |
| I-636 | Ac-PL3-Asp-Ile-B5-Asp-Glu-Ala-Ala-Phe-Gln-S8-Phe-BztA-Tyr-NH2 | 418 |
| I-637 | Ac-PL3-Asp-Ile-B5-Asp-Glu-Ala-Ala-Phe-Gln-S8-Phe-BztA-Pro-NH2 | 419 |
| I-638 | Ac-PL3-Asp-Ile-B5-Asp-Glu-Ala-Ala-Phe-Gln-S8-Phe-BztA-Pro-NH2 | 419 |
| I-639 | Ac-PL3-Asp-Ile-B5-Asp-Glu-Ala-Ala-Phe-Gln-S8-Phe-BztA-Ser-NH2 | 420 |
| I-640 | Ac-PL3-Asp-Ile-B5-Asp-Glu-Ala-Ala-Phe-Gln-S8-Phe-BztA-Ser-NH2 | 420 |
| I-641 | Ac-PL3-Asp-Ile-B5-Asp-Glu-Ala-Ala-Phe-Gln-S8-Phe-BztA-Thr-NH2 | 421 |
| I-642 | Ac-PL3-Asp-Ile-B5-Asp-Glu-Ala-Ala-Phe-Gln-S8-Phe-BztA-Thr-NH2 | 421 |
| I-643 | Ac-PL3-Asp-Ile-B5-Asp-Glu-Ala-Ala-Phe-Gln-S8-Phe-BztA-Trp-NH2 | 422 |
| I-644 | Ac-PL3-Asp-Ile-B5-Asp-Glu-Ala-Ala-Phe-Gln-S8-Phe-BztA-Trp-NH2 | 422 |
| I-645 | Ac-PL3-Asp-Ile-B5-Asp-Glu-Ala-Ala-Phe-Gln-S8-Phe-BztA-Val-NH2 | 423 |
| I-646 | Ac-PL3-Asp-Ile-B5-Asp-Glu-Ala-Ala-Phe-Gln-S8-Phe-BztA-Val-NH2 | 423 |
| I-647 | Ac-PL3-Asp-Ile-B5-Asp-Glu-Leu-Ala-Phe-Gln-S8-Phe-BztA-Gln-NH2 | 424 |
| I-648 | Ac-PL3-Asp-Ile-B5-Asp-Glu-Leu-Ala-Phe-Gln-S8-Phe-BztA-Gln-NH2 | 424 |
| I-649 | Ac-PL3-Asp-Ile-B5-Asp-Glu-Phe-Ala-Phe-Gln-S8-Phe-BztA-Gln-NH2 | 425 |
| I-650 | Ac-PL3-Asp-Ile-B5-Asp-Glu-Phe-Ala-Phe-Gln-S8-Phe-BztA-Gln-NH2 | 425 |
| I-651 | Ac-PL3-Asp-Ile-B5-Asp-Glu-Ser-Ala-Phe-Gln-S8-Phe-BztA-Gln-NH2 | 426 |
| I-652 | Ac-PL3-Asp-Ile-B5-Asp-Glu-Ser-Ala-Phe-Gln-S8-Phe-BztA-Gln-NH2 | 426 |
| I-653 | Ac-PL3-Asp-Ile-B5-Asp-Glu-Gln-Ala-Phe-Gln-S8-Phe-BztA-Gln-NH2 | 427 |
| I-654 | Ac-PL3-Asp-Ile-B5-Asp-Glu-Gln-Ala-Phe-Gln-S8-Phe-BztA-Gln-NH2 | 427 |
| I-655 | Ac-PL3-Asp-Ile-B5-Asp-Glu-Trp-Ala-Phe-Gln-S8-Phe-BztA-Gln-NH2 | 428 |
| I-656 | Ac-PL3-Asp-Ile-B5-Asp-Glu-nLeu-Ala-Phe-Gln-S8-Phe-BztA-Gln-NH2 | 429 |
| I-657 | Ac-PL3-Asp-Ile-B5-Asp-Glu-nLeu-Ala-Phe-Gln-S8-Phe-BztA-Gln-NH2 | 429 |
| I-658 | Ac-PL3-Asp-Ile-B5-Asp-Glu-Ala-Leu-Phe-Gln-S8-Phe-BztA-Gln-NH2 | 430 |
| I-659 | Ac-PL3-Asp-Ile-B5-Asp-Glu-Ala-Leu-Phe-Gln-S8-Phe-BztA-Gln-NH2 | 430 |
| I-660 | Ac-PL3-Asp-Ile-B5-Asp-Glu-Ala-Phe-Phe-Gln-S8-Phe-BztA-Gln-NH2 | 431 |
| I-661 | Ac-PL3-Asp-Ile-B5-Asp-Glu-Ala-Ser-Phe-Gln-S8-Phe-BztA-Gln-NH2 | 432 |
| I-662 | Ac-PL3-Asp-Ile-B5-Asp-Glu-Ala-Ser-Phe-Gln-S8-Phe-BztA-Gln-NH2 | 432 |
| I-663 | Ac-PL3-Asp-Ile-B5-Asp-Glu-Ala-Gln-Phe-Gln-S8-Phe-BztA-Gln-NH2 | 433 |
| I-664 | Ac-PL3-Asp-Ile-B5-Asp-Glu-Ala-Trp-Phe-Gln-S8-Phe-BztA-Gln-NH2 | 434 |
| I-665 | Ac-PL3-Asp-Ile-B5-Asp-Glu-Ala-Trp-Phe-Gln-S8-Phe-BztA-Gln-NH2 | 434 |
| I-666 | Ac-PL3-Asp-Ile-B5-Asp-Glu-Ala-nLeu-Phe-Gln-S8-Phe-BztA-Gln-NH2 | 435 |
| I-667 | Ac-PL3-Asp-Ile-B5-Asp-Glu-Ala-nLeu-Phe-Gln-S8-Phe-BztA-Gln-NH2 | 435 |
| I-668 | Ac-PL3-isoDAsp-Ile-B5-Asp-Glu-Ala-Ala-Phe-Gln-S8-Phe-BztA-Gln-NH2 | 436 |
| I-669 | Ac-PL3-isoDAsp-Ile-B5-Asp-Glu-Ala-Ala-Phe-Gln-S8-Phe-BztA-Gln-NH2 | 436 |
| I-670 | Ac-PL3-RbGlu-Ile-B5-Asp-Glu-Ala-Ala-Phe-Gln-S8-Phe-BztA-Gln-NH2 | 437 |
| I-671 | Ac-PL3-RbGlu-Ile-B5-Asp-Glu-Ala-Ala-Phe-Gln-S8-Phe-BztA-Gln-NH2 | 437 |
| I-672 | Ac-PL3-SbGlu-Ile-B5-Asp-Glu-Ala-Ala-Phe-Gln-S8-Phe-BztA-Gln-NH2 | 438 |
| I-673 | Ac-PL3-Asp-Ile-B5-isoAsp-Glu-Ala-Ala-Phe-Gln-S8-Phe-BztA-Gln-NH2 | 439 |
| I-674 | Ac-PL3-Asp-Ile-B5-isoDAsp-Glu-Ala-Ala-Phe-Gln-S8-Phe-BztA-Gln-NH2 | 440 |
| I-675 | Ac-PL3-Asp-Ile-B5-RbGlu-Glu-Ala-Ala-Phe-Gln-S8-Phe-BztA-Gln-NH2 | 441 |
| I-676 | Ac-PL3-Asp-Ile-B5-SbGlu-Glu-Ala-Ala-Phe-Gln-S8-Phe-BztA-Gln-NH2 | 442 |
| I-677 | Ac-PL3-Asp-Npg-B5-Asp-Glu-Ala-Ala-Phe-Asn-S8-Phe-BztA-Gln-NH2 | 443 |
| I-678 | Ac-PL3-Asp-Npg-B5-Asp-Asp-Ala-Ala-Phe-Asn-S8-3Thi-BztA-Gln-NH2 | 444 |
| I-679 | Ac-PL3-Asp-Npg-B5-Asp-Asp-Ala-Ala-Phe-Asn-S8-3Thi-BztA-Gln-NH2 | 444 |
| I-680 | Ac-PL3-Asp-Leu-B5-Asp-Asp-Ala-Ala-Phe-Asn-S8-3Thi-BztA-Gln-NH2 | 445 |
| I-681 | Ac-PL3-Asp-Leu-B5-Asp-Asp-Ala-Ala-Phe-Asn-S8-3Thi-BztA-Gln-NH2 | 445 |
| I-682 | Ac-PL3-Asp-Npg-B5-Asp-Glu-Ala-Ala-Phe-Asn-S8-3Thi-BztA-Gln-NH2 | 446 |
| I-683 | Ac-PL3-Asp-Npg-B5-Asp-Glu-Ala-Ala-Phe-Asn-S8-3Thi-BztA-Gln-NH2 | 446 |
| I-684 | Ac-PL3-Asp-Npg-B5-Asp-Glu-Ala-Ala-Phe-Asn-PyrS2-Phe-BztA-Gln-NH2 | 447 |
| I-685 | Ac-PL3-Asp-Npg-B5-Asp-Glu-Ala-Ala-Phe-Asn-PyrS2-Phe-BztA-Gln-NH2 | 447 |
| I-686 | Ac-PL3-Asp-Npg-B5-Asp-Asp-Ala-Ala-Phe-Asn-PyrS2-3Thi-BztA-Gln-NH2 | 448 |
| I-687 | Ac-PL3-Asp-Npg-B5-Asp-Asp-Ala-Ala-Phe-Asn-PyrS2-3Thi-BztA-Gln-NH2 | 448 |
| I-688 | Ac-PL3-Asp-Leu-B5-Asp-Asp-Ala-Ala-Phe-Asn-PyrS2-3Thi-BztA-Gln-NH2 | 449 |
| I-689 | Ac-PL3-Asp-Leu-B5-Asp-Asp-Ala-Ala-Phe-Asn-PyrS2-3Thi-BztA-Gln-NH2 | 449 |
| I-690 | Ac-PL3-Asp-Npg-B5-Asp-Glu-Ala-Ala-Phe-Asn-PyrS2-3Thi-BztA-Gln-NH2 | 450 |
| I-691 | Ac-PL3-Asp-Npg-B5-Asp-Glu-Ala-Ala-Phe-Asn-PyrS2-3Thi-BztA-Gln-NH2 | 450 |
| I-692 | Ac-PL3-Asp-Npg-B5-Asp-Glu-Ala-Ala-Phe-Asn-PyrS2-Phe-BztA-His-NH2 | 451 |
| I-693 | Ac-PL3-Asp-Npg-B5-Asp-Glu-Ala-Ala-Phe-Asn-PyrS2-Phe-BztA-His-NH2 | 451 |
| I-694 | Ac-PL3-Asp-Npg-B5-Asp-Asp-Ala-Ala-Phe-Asn-PyrS2-3Thi-BztA-His-NH2 | 452 |
| I-695 | Ac-PL3-Asp-Npg-B5-Asp-Asp-Ala-Ala-Phe-Asn-PyrS2-3Thi-BztA-His-NH2 | 452 |
| I-696 | Ac-PL3-Asp-Leu-B5-Asp-Asp-Ala-Ala-Phe-Asn-PyrS2-3Thi-BztA-His-NH2 | 453 |
| I-697 | Ac-PL3-Asp-Npg-B5-Asp-Glu-Ala-Ala-Phe-Asn-PyrS2-3Thi-BztA-His-NH2 | 454 |
| I-698 | Ac-PL3-Asp-Npg-B5-Asp-Glu-Ala-Ala-Phe-Asn-PyrS2-3Thi-BztA-His-NH2 | 454 |
| I-699 | Ac-PL3-Asp-Ile-B5-Asp-TfeGA-Ala-Ala-Phe-Asn-S8-Phe-BztA-Gln-NH2 | 455 |
| I-700 | Ac-PL3-Asp-Ile-B5-Asp-TfeGA-Ala-Ala-Phe-Asn-S8-3Thi-BztA-Gln-NH2 | 456 |
| I-701 | Ac-PL3-Asp-Ile-B5-Asp-TfeGA-Ala-Ala-Phe-Asn-S8-3Thi-BztA-Gln-NH2 | 456 |
| I-702 | Ac-PL3-Asp-Ile-B5-Asp-TfeGA-Ala-Ala-Phe-Asn-S8-3Thi-BztA-His-NH2 | 457 |
| I-703 | Ac-PL3-Asp-Npg-B5-Asp-TfeGA-Ala-Ala-Phe-Asn-S8-Phe-BztA-Gln-NH2 | 458 |
| I-704 | Ac-PL3-Asp-Npg-B5-Asp-TfeGA-Ala-Ala-Phe-Asn-S8-3Thi-BztA-Gln-NH2 | 459 |
| I-705 | Ac-PL3-Asp-Npg-B5-Asp-TfeGA-Ala-Ala-Phe-Asn-S8-3Thi-BztA-His-NH2 | 460 |
| I-706 | Ac-PL3-Asp-Cha-B5-Asp-TfeGA-Ala-Ala-Phe-Asn-S8-Phe-BztA-Gln-NH2 | 461 |
| I-707 | Ac-PL3-Asp-Cha-B5-Asp-TfeGA-Ala-Ala-Phe-Asn-S8-3Thi-BztA-Gln-NH2 | 462 |
| I-708 | Ac-PL3-Asp-Cha-B5-Asp-TfeGA-Ala-Ala-Phe-Asn-S8-3Thi-BztA-Gln-NH2 | 462 |
| I-709 | Ac-PL3-Asp-Cha-B5-Asp-TfeGA-Ala-Ala-Phe-Asn-S8-3Thi-BztA-His-NH2 | 463 |
| I-710 | MeSO2-PL3-Asp-Ile-B5-Asp-Glu-Ala-Ala-Phe-Gln-S8-Phe-BztA-Gln-NH2 | 464 |
| I-711 | MeSO2-PL3-Asp-Ile-B5-Asp-Glu-Ala-Ala-Phe-Gln-S8-Phe-BztA-Gln-NH2 | 464 |
| I-712 | Ac-PL3-Asp-Npg-B5-Asp-Glu-Ala-Ala-Phe-Asn-S8-Phe-BztA-NH2 | 465 |
| I-713 | Ac-PL3-Asp-Npg-B5-Asp-Glu-Ala-Ala-Phe-Asn-S8-Phe-BztA-NH2 | 465 |
| I-714 | Ac-PL3-Asp-Ile-B5-Asp-Glu-Ala-Ala-Phe-Asn-S8-Phe-BztA-NH2 | 466 |
| I-715 | Ac-PL3-Asp-Ile-B5-Asp-Glu-Ala-Ala-Phe-Asn-S8-Phe-BztA-NH2 | 466 |
| I-716 | Ac-PL3-Asp-Leu-B5-Asp-Glu-Ala-Ala-Phe-Asn-S8-3Thi-BztA-NH2 | 467 |
| I-717 | Ac-PL3-Asp-Leu-B5-Asp-Glu-Ala-Ala-Phe-Asn-S8-3Thi-BztA-NH2 | 467 |
| I-718 | Ac-PL3-Asp-Npg-B5-Asp-Glu-Ala-Ala-Phe-Asn-S8-3Thi-BztA-NH2 | 468 |
| I-719 | Ac-PL3-Asp-Npg-B5-Asp-Glu-Ala-Ala-Phe-Asn-S8-3Thi-BztA-NH2 | 468 |
| I-720 | Ac-PL3-Asp-Ile-B5-Asp-Glu-Ala-Ala-Phe-Asn-S8-3Thi-BztA-NH2 | 469 |
| I-721 | Ac-PL3-Asp-Ile-B5-Asp-Glu-Ala-Ala-Phe-Asn-S8-3Thi-BztA-NH2 | 469 |
| I-722 | Ac-PL3-Asp-Ile-B5-Asp-Glu-Ala-Ala-Cha-Gln-S8-Phe-BztA-Gln-NH2 | 470 |
| I-723 | Ac-PL3-Asp-Ile-B5-Asp-Glu-Ala-Ala-Bip-Gln-S8-Phe-BztA-Gln-NH2 | 471 |
| I-724 | Ac-PL3-Asp-Ile-B5-Asp-Glu-Ala-Ala-3OMeF-Gln-S8-Phe-BztA-Gln-NH2 | 472 |
| I-725 | Ac-PL3-Asp-Ile-B5-Asp-Glu-Ala-Ala-DipA-Gln-S8-Phe-BztA-Gln-NH2 | 473 |
| I-726 | Ac-PL3-Asp-Ile-B5-Asp-Glu-Ala-Ala-Phg-Gln-S8-Phe-BztA-Gln-NH2 | 474 |
| I-727 | Ac-PL3-Asp-Ile-B5-Asp-Glu-Ala-Ala-Npg-Gln-S8-Phe-BztA-Gln-NH2 | 475 |
| I-728 | Ac-PL3-Asp-Ile-B5-Asp-Glu-Ala-Ala-nLeu-Gln-S8-Phe-BztA-Gln-NH2 | 476 |
| I-729 | Ac-PL3-Asp-Leu-B5-Asp-Glu-Ala-Ala-Phe-Asn-S8-Phe-BztA-Gln-Ala-NH2 | 477 |
| I-730 | Ac-PL3-Asp-Leu-B5-Asp-Glu-Ala-Ala-Phe-Asn-S8-Phe-BztA-Gln-Ala-NH2 | 477 |
| I-731 | Ac-PL3-Asp-Leu-B5-Asp-Glu-Ala-Ala-Phe-Asn-S8-Phe-BztA-Gln-dAla-NH2 | 478 |
| I-732 | Ac-PL3-Asp-Leu-B5-Asp-Glu-Ala-Ala-Phe-Asn-S8-Phe-BztA-Gln-Ile-NH2 | 479 |
| I-733 | Ac-PL3-Asp-Leu-B5-Asp-Glu-Ala-Ala-Phe-Asn-S8-Phe-BztA-Gln-Ile-NH2 | 479 |
| I-734 | Ac-PL3-Asp-Leu-B5-Asp-Glu-Ala-Ala-Phe-Asn-S8-Phe-BztA-Gln-dIle-NH2 | 480 |
| I-735 | Ac-PL3-Asp-Leu-B5-Asp-Glu-Ala-Ala-Phe-Asn-S8-Phe-BztA-Gln-dIle-NH2 | 480 |
| I-736 | Ac-PL3-Asp-Leu-B5-Asp-Glu-Ala-Ala-Phe-Asn-S8-Phe-BztA-Gln-Aib-NH2 | 481 |
| I-737 | Ac-PL3-Asp-Leu-B5-Asp-Glu-Ala-Ala-Phe-Asn-S8-Phe-BztA-Gln-Aib-NH2 | 481 |
| I-738 | Ac-PL3-Asp-Leu-B5-Asp-Glu-Ala-Ala-Phe-Asn-S8-Phe-BztA-His-Ala-NH2 | 482 |
| I-739 | Ac-PL3-Asp-Leu-B5-Asp-Glu-Ala-Ala-Phe-Asn-S8-Phe-BztA-His-dAla-NH2 | 483 |
| I-740 | Ac-PL3-Asp-Leu-B5-Asp-Glu-Ala-Ala-Phe-Asn-S8-Phe-BztA-His-Ile-NH2 | 484 |
| I-741 | Ac-PL3-Asp-Leu-B5-Asp-Glu-Ala-Ala-Phe-Asn-S8-Phe-BztA-His-Ile-NH2 | 484 |
| I-742 | Ac-PL3-Asp-Leu-B5-Asp-Glu-Ala-Ala-Phe-Asn-S8-Phe-BztA-His-dIle-NH2 | 485 |
| I-743 | Ac-PL3-Asp-Leu-B5-Asp-Glu-Ala-Ala-Phe-Asn-S8-Phe-BztA-His-Aib-NH2 | 486 |
| I-744 | Ac-PL3-Asp-Leu-B5-Asp-Glu-Ala-Ala-Phe-Asn-S8-Phe-BztA-dHis-NH2 | 487 |
| I-745 | Ac-PL3-Asp-Leu-B5-Asp-Glu-Ala-Ala-Phe-Asn-S8-Phe-BztA-dHis-NH2 | 487 |
| I-746 | Ac-PL3-Asp-Leu-B5-Asp-Glu-Ala-Ala-Phe-Asn-S8-Phe-BztA-NMeHis-NH2 | 488 |
| I-747 | Ac-PL3-Asp-Leu-B5-Asp-Glu-Ala-Ala-Phe-Asn-S8-Phe-BztA-NMeHis-NH2 | 488 |
| I-748 | Ac-PL3-Asp-Leu-B5-Asp-Glu-Ala-Ala-Phe-Asn-S8-Phe-BztA-Thr-NH2 | 489 |
| I-749 | Ac-PL3-Asp-Leu-B5-Asp-Glu-Ala-Ala-Phe-Asn-S8-Phe-BztA-Thr-NH2 | 489 |
| I-750 | Ac-PL3-Asp-Leu-B5-Asp-Glu-Ala-Ala-Phe-Asn-S8-Phe-BztA-Ser-NH2 | 490 |
| I-751 | Ac-PL3-Asp-Leu-B5-Asp-Glu-Ala-Ala-Phe-Asn-S8-Phe-BztA-Ser-NH2 | 490 |
| I-752 | Ac-PL3-Asp-Leu-B5-Asp-Glu-Ala-Ala-Phe-Asn-S8-Phe-BztA-Asn-NH2 | 491 |
| I-753 | Ac-PL3-Asp-Leu-B5-Asp-Glu-Ala-Ala-Phe-Asn-S8-Phe-BztA-Asn-NH2 | 491 |
| I-754 | Ac-PL3-Asp-Leu-B5-Asp-Glu-Ala-Ala-Phe-Asn-S8-Phe-BztA-dThr-NH2 | 492 |
| I-755 | Ac-PL3-Asp-Leu-B5-Asp-Glu-Ala-Ala-Phe-Asn-S8-Phe-BztA-dThr-NH2 | 492 |
| I-756 | Ac-PL3-Asp-Leu-B5-Asp-Glu-Ala-Ala-Phe-Asn-S8-Phe-BztA-dSer-NH2 | 493 |
| I-757 | Ac-PL3-Asp-Leu-B5-Asp-Glu-Ala-Ala-Phe-Asn-S8-Phe-BztA-dAsn-NH2 | 494 |
| I-758 | Ac-PL3-Asp-Leu-B5-Asp-Glu-Ala-Ala-Phe-Asn-S8-Phe-BztA-dAsn-NH2 | 494 |
| I-759 | Ac-PL3-Asp-Leu-B5-Asp-Glu-Ala-Ala-Phe-Asn-S8-Phe-BztA-NMeS-NH2 | 495 |
| I-760 | Ac-PL3-Asp-Leu-B5-Asp-Glu-Ala-Ala-Phe-Asn-S8-Phe-BztA-NMeS-NH2 | 495 |
| I-761 | Ac-PL3-Asp-Npg-B5-Asp-TfeGA-Ala-Ala-Phe-Asn-PyrS2-3Thi-BztA-Gln-NH2 | 496 |
| I-762 | Ac-PL3-Asp-Npg-B5-Asp-TfeGA-Ala-Ala-Phe-Asn-PyrS2-3Thi-BztA-Gln-NH2 | 496 |
| I-763 | Ac-PL3-Asp-Npg-B5-Asp-Asp-Ala-Ala-Phe-Gln-PyrS2-3Thi-BztA-Gln-NH2 | 497 |
| I-764 | Ac-PL3-Asp-Npg-B5-Asp-Asp-Ala-Ala-Phe-Gln-PyrS2-3Thi-BztA-Gln-NH2 | 497 |
| I-765 | Ac-PL3-Asp-Npg-B5-Asp-Asp-Ala-Ala-Phe-Asn-SeN-3Thi-BztA-Gln-NH2 | 498 |
| I-766 | Ac-PL3-Asp-Npg-B5-Asp-Asp-Ala-Ala-Phe-Asn-SeN-3Thi-BztA-Gln-NH2 | 498 |
| I-767 | Ac-PL3-Asp-Npg-B5-Asp-Asp-Ala-Ala-Phe-Asn-PyrS3-3Thi-BztA-Gln-NH2 | 499 |
| I-768 | Ac-PL3-Asp-Npg-B5-Asp-Asp-Ala-Ala-Phe-Asn-PyrS3-3Thi-BztA-Gln-NH2 | 499 |
| I-769 | Ac-PL3-Asp-Npg-B5-Asp-TfeGA-Ala-Ala-Phe-Asn-PyrS2-3Thi-BztA-His-NH2 | 500 |
| I-770 | Ac-PL3-Asp-Npg-B5-Asp-TfeGA-Ala-Ala-Phe-Asn-PyrS2-3Thi-BztA-His-NH2 | 500 |
| I-771 | Ac-PL3-Asp-Npg-B5-Asp-Asp-Ala-Ala-Phe-Gln-PyrS2-3Thi-BztA-His-NH2 | 501 |
| I-772 | Ac-PL3-Asp-Npg-B5-Asp-Asp-Ala-Ala-Phe-Gln-PyrS2-3Thi-BztA-His-NH2 | 501 |
| I-773 | Ac-PL3-Asp-Npg-B5-Asp-Asp-Ala-Ala-Phe-Asn-SeN-3Thi-BztA-His-NH2 | 502 |
| I-774 | Ac-PL3-Asp-Npg-B5-Asp-Asp-Ala-Ala-Phe-Asn-SeN-3Thi-BztA-His-NH2 | 502 |
| I-775 | Ac-PL3-Asp-Npg-B5-Asp-Asp-Ala-Ala-Phe-Asn-PyrS3-3Thi-BztA-His-NH2 | 503 |
| I-776 | Ac-PL3-Asp-Npg-B5-Asp-Asp-Ala-Ala-Phe-Asn-PyrS3-3Thi-BztA-His-NH2 | 503 |
| I-777 | Ac-PL3-Asp-Npg-B5-Asp-Asp-Ala-Ala-Phe-Asn-PyrS2-Phe-BztA-His-NH2 | 504 |
| I-778 | Ac-PL3-Asp-Npg-B5-Asp-Asp-Ala-Ala-Phe-Asn-PyrS2-2Thi-BztA-Gln-NH2 | 505 |
| I-779 | Ac-PL3-Asp-Npg-B5-Asp-Asp-Ala-Ala-Phe-Asn-PyrS2-2Thi-BztA-His-NH2 | 506 |
| I-780 | Ac-PL3-Asp-Npg-B5-Asp-Asp-Ala-Ala-Phe-Asn-PyrS2-2OMeF-BztA-Gln-NH2 | 507 |
| I-781 | Ac-PL3-Asp-Npg-B5-Asp-Asp-Ala-Ala-Phe-Asn-PyrS2-2OMeF-BztA-His-NH2 | 508 |
| I-782 | Ac-PL3-Asp-Npg-B5-Asp-Asp-Ala-Ala-Phe-Asn-PyrS2-2MeF-BztA-His-NH2 | 509 |
| I-783 | Ac-PL3-Asp-Npg-B5-Asp-Asp-Ala-Ala-Phe-Asn-PyrS2-2C1F-BztA-Gln-NH2 | 510 |
| I-784 | Ac-PL3-Asp-Npg-B5-Asp-Asp-Ala-Ala-Phe-Asn-PyrS2-2C1F-BztA-His-NH2 | 511 |
| I-785 | Ac-PL3-Asp-Npg-B5-Asp-Asp-Ala-Ala-Phe-Asn-PyrS2-3Thi-BztA-NH2 | 512 |
| I-786 | Ac-PL3-Asp-Npg-B5-Asp-Glu-Ala-Ala-Phe-Asn-PyrS2-3Thi-BztA-NH2 | 513 |
| I-787 | Ac-PL3-Asp-hLeu-B5-Asp-Asp-Ala-Ala-Phe-Asn-PyrS2-3Thi-BztA-Gln-NH2 | 514 |
| I-788 | Ac-PL3-Asp-hLeu-B5-Asp-Asp-Ala-Ala-Phe-Asn-PyrS2-3Thi-BztA-His-NH2 | 515 |
| I-789 | Ac-PL3-Asp-Npg-B5-Asp-Asp-Ala-Ala-Phe-Asn-PyrS2-3Thi-1NapA-His-NH2 | 516 |
| I-790 | Ac-PL3-Asp-Npg-B5-Asp-Asp-Ala-Ala-Phe-Asn-PyrS2-3Thi-2NapA-His-NH2 | 517 |
| I-791 | Ac-PL3-Asp-Npg-B5-Asp-Asp-Ala-Ala-Phe-Asn-PyrS2-3Thi-5C1W-His-NH2 | 518 |
| I-792 | Ac-PL3-Asp-Npg-B5-Asp-Asp-Ala-Ala-Phe-Asn-PyrS2-3Thi-Trp-His-NH2 | 519 |
| I-793 | Ac-PL3-Asp-Npg-B5-Asp-Asp-Ala-Ala-Phe-Asn-PyrS2-BztA-BztA-His-NH2 | 520 |
| I-794 | Ac-PL3-Asp-Npg-B5-Asp-Asp-Ala-Ala-Phe-Asn-PyrS2-1NapA-BztA-His-NH2 | 521 |
| I-795 | Ac-PL3-Asp-Npg-B5-Asp-Asp-Ala-Ala-Phe-Asn-PyrS2-2NapA-BztA-His-NH2 | 522 |
| I-796 | Ac-PL3-Phe-Npg-B5-Asp-Asp-Ala-Ala-Asp-Asn-PyrS2-3Thi-BztA-His-NH2 | 523 |
| I-797 | Ac-PL3-Phe-Npg-B5-Asp-Asp-Ala-Ala-Asp-Asn-PyrS2-3Thi-BztA-Gln-NH2 | 524 |
| I-798 | Ac-PL3-Asp-Npg-B5-Asp-Asp-Ala-Ala-Phe-Asn-PyrS2-3Thi-6C1W-His-NH2 | 525 |
| I-799 | Ac-PL3-Asp-Npg-B5-Asp-Asp-Ala-Ala-Phe-Asn-PyrS2-3Thi-1MeW-His-NH2 | 526 |
| I-800 | Ac-PL3-Asp-Npg-B5-Asp-Asp-Ala-Ala-Phe-Asn-PyrS2-3Thi-5FW-His-NH2 | 527 |
| I-801 | Ac-PL3-Asp-Npg-B5-Asp-Asp-Ala-Ala-Phe-Leu-PyrS2-3Thi-BztA-Gln-NH2 | 528 |
| I-802 | Ac-PL3-Asp-Npg-B5-Asp-Asp-Ala-Ala-Phe-Leu-PyrS2-3Thi-BztA-His-NH2 | 529 |
| I-803 | Ac-PL3-Asp-Npg-B5-Asp-Asp-Ala-Ala-Phe-Hse-PyrS2-3Thi-BztA-Gln-NH2 | 530 |
| I-804 | Ac-PL3-Asp-Npg-B5-Asp-Asp-Ala-Ala-Phe-Hse-PyrS2-3Thi-BztA-His-NH2 | 531 |
| I-805 | Ac-PL3-Asp-Npg-B5-Asp-Asp-Ala-Ala-Phe-Cit-PyrS2-3Thi-BztA-Gln-NH2 | 532 |
| I-806 | Ac-PL3-Asp-Npg-B5-Asp-Asp-Ala-Ala-Phe-Cit-PyrS2-3Thi-BztA-His-NH2 | 533 |
| I-807 | Ac-PL3-Asp-Npg-B5-Asp-Asp-Ala-Ala-Phe-Phe-PyrS2-3Thi-BztA-Gln-NH2 | 534 |
| I-808 | Ac-PL3-Asp-Npg-B5-Asp-Asp-Ala-Ala-Phe-Phe-PyrS2-3Thi-BztA-His-NH2 | 535 |
| I-809 | Ac-PL3-Asp-Npg-B5-Asp-Asp-Ala-Ala-Phe-Thr-PyrS2-3Thi-BztA-Gln-NH2 | 536 |
| I-810 | Ac-PL3-Asp-Npg-B5-Asp-Asp-Ala-Ala-Phe-Thr-PyrS2-3Thi-BztA-His-NH2 | 537 |
| I-811 | Ac-PL3-Asp-Npg-B5-Asp-Asp-Ala-Ala-Phe-Asn-PyrS2-4Thz-BztA-Gln-NH2 | 538 |
| I-812 | Ac-PL3-Asp-Npg-B5-Asp-Asp-Ala-Ala-4Thz-Asn-PyrS2-3Thi-BztA-Gln-NH2 | 539 |
| I-813 | Ac-PL3-Asp-Npg-B5-Asp-Asp-Ala-Ala-Phe-Asn-PyrS2-3Thi-BztA-dHis-NH2 | 540 |
| I-814 | Ac-PL3-Asp-Npg-B5-Asp-Asp-Ala-Ala-Phe-Asn-PyrS2-3Thi-BztA-Thr-NH2 | 541 |
| I-815 | Ac-PL3-Asp-Npg-B5-Asp-Asp-Ala-Ala-Phe-Asn-PyrS2-3Thi-BztA-Ser-NH2 | 542 |
| I-816 | Ac-PL3-Asp-Npg-B5-Asp-Asp-Ala-Ala-Phe-Asn-PyrS2-3Thi-BztA-Asn-NH2 | 543 |
| I-817 | Ac-PL3-Asp-Npg-B5-Asp-Asp-Ala-Ala-Phe-Asn-PyrS2-3Thi-BztA-dThr-NH2 | 544 |
| I-818 | Ac-PL3-Asp-Npg-B5-Asp-Asp-Ala-Ala-Phe-Asn-PyrS2-3Thi-BztA-dAsn-NH2 | 545 |
| I-819 | Ac-PL3-Asp-Npg-B5-Asp-Asp-Ala-Ala-Phe-Asn-PyrS2-3Thi-BztA-Gln-Ala-NH2 | 546 |
| I-820 | Ac-PL3-Asp-Npg-B5-Asp-Asp-Ala-Ala-Phe-Asn-PyrS2-3Thi-BztA-His-Ala-NH2 | 547 |
| I-821 | Ac-PL3-Asp-Npg-B5-Asp-Asp-Ala-Ala-Phe-Asn-PyrS2-3Thi-BztA-Asn-Ala-NH2 | 548 |
| I-822 | Ac-PL3-Asp-Npg-B5-Asp-Asp-Ala-Ala-Phe-Asn-PyrS2-3Thi-BztA-Gln-dAla-NH2 | 549 |
| I-823 | Ac-PL3-Asp-Npg-B5-Asp-Asp-Ala-Ala-Phe-Asn-PyrS2-3Thi-BztA-His-dAla-NH2 | 550 |
| I-824 | Ac-PL3-Asp-Npg-B5-Asp-Asp-Ala-Ala-Phe-Asn-PyrS2-3Thi-BztA-Asn-dAla-NH2 | 551 |
| I-825 | Ac-PL3-Asp-Npg-B5-Asp-Asp-Ala-Ala-Phe-Asn-PyrS3-3Thi-BztA-Gln-Ala-NH2 | 552 |
| I-826 | Ac-PL3-Asp-Npg-B5-Asp-Asp-Ala-Ala-Phe-Asn-PyrS3-3Thi-BztA-Gln-Ala-NH2 | 552 |
| I-827 | Ac-PL3-Asp-Npg-B5-Asp-Asp-Ala-Ala-Phe-Asn-PyrS3-3Thi-BztA-His-Ala-NH2 | 553 |
| I-828 | Ac-PL3-Asp-Npg-B5-Asp-Asp-Ala-Ala-Phe-Asn-PyrS3-3Thi-BztA-His-Ala-NH2 | 553 |
| I-829 | Ac-PL3-Asp-Npg-B5-Asp-Asp-Ala-Ala-Phe-Asn-PyrS3-3Thi-BztA-Asn-Ala-NH2 | 554 |
| I-830 | Ac-PL3-Asp-Npg-B5-Asp-Asp-Ala-Ala-Phe-Asn-PyrS3-3Thi-BztA-Gln-dAla-NH2 | 555 |
| I-831 | Ac-PL3-Asp-Npg-B5-Asp-Asp-Ala-Ala-Phe-Asn-PyrS3-3Thi-BztA-Gln-dAla-NH2 | 555 |
| I-832 | Ac-PL3-Asp-Npg-B5-Asp-Asp-Ala-Ala-Phe-Asn-PyrS3-3Thi-BztA-His-dAla-NH2 | 556 |
| I-833 | Ac-PL3-Asp-Npg-B5-Asp-Asp-Ala-Ala-Phe-Asn-PyrS3-3Thi-BztA-His-dAla-NH2 | 556 |
| I-834 | Ac-PL3-Asp-Npg-B5-Asp-Asp-Ala-Ala-Phe-Asn-PyrS3-3Thi-BztA-Asn-dAla-NH2 | 557 |
| I-835 | Ac-PL3-Asp-Npg-B5-Asp-Asp-Ala-Ala-Phe-Asn-PyrS3-3Thi-BztA-Asn-dAla-NH2 | 557 |
| I-836 | Ac-PL3-Asp-Npg-B5-Asp-Asn-Ala-Ala-Phe-Asn-PyrS2-3Thi-BztA-Gln-NH2 | 558 |
| I-837 | Ac-PL3-Asp-Npg-B5-Asp-Asn-Ala-Ala-Phe-Asn-PyrS2-3Thi-BztA-His-NH2 | 559 |
| I-838 | Ac-PL3-Asp-Npg-B5-Asp-Gln-Ala-Ala-Phe-Asn-PyrS2-3Thi-BztA-Gln-NH2 | 560 |
| I-839 | Ac-PL3-Asp-Npg-B5-Asp-Gln-Ala-Ala-Phe-Asn-PyrS2-3Thi-BztA-His-NH2 | 561 |
| I-840 | Ac-PL3-Asp-Npg-B5-Asp-Cit-Ala-Ala-Phe-Asn-PyrS2-3Thi-BztA-Gln-NH2 | 562 |
| I-841 | Ac-PL3-Asp-Npg-B5-Asp-Cit-Ala-Ala-Phe-Asn-PyrS2-3Thi-BztA-His-NH2 | 563 |
| I-842 | Ac-PL3-Asp-Npg-B5-Asp-Tyr-Ala-Ala-Phe-Asn-PyrS2-3Thi-BztA-Gln-NH2 | 564 |
| I-843 | Ac-PL3-Asp-Npg-B5-Asp-Tyr-Ala-Ala-Phe-Asn-PyrS2-3Thi-BztA-His-NH2 | 565 |
| I-844 | Ac-PL3-Asp-Npg-B5-Asp-Thr-Ala-Ala-Phe-Asn-PyrS2-3Thi-BztA-Gln-NH2 | 566 |
| I-845 | Ac-PL3-Asp-Npg-B5-Asp-Thr-Ala-Ala-Phe-Asn-PyrS2-3Thi-BztA-His-NH2 | 567 |
| I-846 | Ac-PL3-Asp-Npg-B5-Asp-Leu-Ala-Ala-Phe-Asn-PyrS2-3Thi-BztA-Gln-NH2 | 568 |
| I-847 | Ac-PL3-Asp-Npg-B5-Asp-Leu-Ala-Ala-Phe-Asn-PyrS2-3Thi-BztA-His-NH2 | 569 |
| I-848 | Ac-PL3-Asp-Npg-B5-Asp-TfeGA-Ala-Ala-Phe-Phe-PyrS2-3Thi-BztA-Gln-NH2 | 570 |
| I-849 | Ac-PL3-Asp-Npg-B5-Asp-TfeGA-Ala-Ala-Phe-Leu-PyrS2-3Thi-BztA-Gln-NH2 | 571 |
| I-850 | Ac-PL3-Asp-Npg-B5-Asp-4COOHF-Ala-Ala-Phe-Phe-PyrS2-3Thi-BztA-Gln-NH2 | 572 |
| I-851 | Ac-PL3-Asp-Npg-B5-Asp-4COOHF-Ala-Ala-Phe-Leu-PyrS2-3Thi-BztA-Gln-NH2 | 573 |
| I-852 | Ac-PL3-Asp-Npg-B5-Asp-3COOHF-Ala-Ala-Phe-Phe-PyrS2-3Thi-BztA-Gln-NH2 | 574 |
| I-853 | Ac-PL3-Asp-Npg-B5-Asp-2COOHF-Ala-Ala-Phe-Phe-PyrS2-3Thi-BztA-Gln-NH2 | 575 |
| I-854 | Ac-PL3-Asp-Npg-B5-Asp-2COOHF-Ala-Ala-Phe-Leu-PyrS2-3Thi-BztA-Gln-NH2 | 576 |
| I-855 | Ac-PL3-Asp-Npg-B5-Asp-Asn-Ala-Ala-Phe-Phe-PyrS2-3Thi-BztA-Gln-NH2 | 577 |
| I-856 | Ac-PL3-Asp-Npg-B5-Asp-Asn-Ala-Ala-Phe-Leu-PyrS2-3Thi-BztA-Gln-NH2 | 578 |
| I-857 | Ac-PL3-Asp-Npg-B5-Asp-Asn-Ala-Ala-Phe-Asn-PyrS2-3Thi-BztA-NH2 | 579 |
| I-858 | Ac-PL3-Asp-Npg-B5-Asp-Gln-Ala-Ala-Phe-Asn-PyrS2-3Thi-BztA-NH2 | 580 |
| I-859 | Ac-PL3-Asp-Npg-B5-Asp-Thr-Ala-Ala-Phe-Asn-PyrS2-3Thi-BztA-NH2 | 581 |
| I-860 | Ac-PL3-Asp-Npg-B5-Asp-Thr-Ala-Ala-Phe-Asn-PyrS2-3Thi-BztA-NH2 | 581 |
| I-861 | Ac-PL3-Asp-Npg-B5-Asp-Cit-Ala-Ala-Phe-Asn-PyrS2-3Thi-BztA-NH2 | 582 |
| I-862 | Ac-PL3-Asp-Npg-B5-Asp-Leu-Ala-Ala-Phe-Asn-PyrS2-3Thi-BztA-NH2 | 583 |
| I-863 | Ac-PL3-Asp-Npg-B5-Asp-His-Ala-Ala-Phe-Asn-PyrS2-3Thi-BztA-NH2 | 584 |
| I-864 | Ac-PL3-Asp-Npg-B5-Asp-His-Ala-Ala-Phe-Asn-PyrS2-3Thi-BztA-Gln-NH2 | 585 |
| I-865 | Ac-PL3-Asp-Npg-B5-Asp-Asp-[FAM6Ppg][1TriAc]Lys-Ala-Phe-Asn-PyrS2-3Thi- | 586 |
| | BztA-His-NH2 | |
| I-866 | Ac-PL3-Asp-Npg-B5-Asp-Asp-Ala-[FAM6Ppg][1TriAc]Lys-Phe-Asn-PyrS2-3Thi- | 587 |
| | BztA-His-NH2 | |
| I-867 | Ac-PL3-Asp-Npg-B5-Asp-Leu-[FAM6Ppg][p1TB]Lys-Ala-Phe-Asn-PyrS2-3Thi-BztA- | 588 |
| | Gln-NH2 | |
| I-868 | Ac-PL3-Asp-Npg-B5-Asp-Leu-[FAM6Ppg][p1TB]Lys-Ala-Phe-Leu-PyrS2-3Thi-BztA- | 589 |
| | Gln-NH2 | |
| I-869 | Ac-PL3-Asp-Npg-B5-Asp-Leu-[FAM6Ppg][p1TB]Lys-Ala-Phe-Phe-PyrS2-3Thi-BztA- | 590 |
| | Gln-NH2 | |
| I-870 | Ac-PL3-Asp-Npg-B5-Asp-Asp-Ala-Ala-Phe-Asn-PyrS2-3Thi-BztA-Gln-Ala-Ala | 591 |
| I-871 | Ac-PL3-Asp-Npg-B5-Asp-Asp-Ala-Ala-Phe-Asn-PyrS2-3Thi-BztA-Gln-dAla-Ala | 592 |
| I-872 | Ac-PL3-Asp-Npg-B5-Asp-Asp-Ala-Ala-Phe-Asn-PyrS2-3Thi-BztA-Gln-dAla-dAla | 593 |
| I-873 | Ac-PL3-Asp-Npg-B5-Asp-Asp-Ala-Ala-Phe-Asn-PyrS2-3Thi-BztA-Gln-Ala-Ala | 591 |
| I-874 | Ac-PL3-Asp-Npg-B5-Asp-Asp-Ala-Ala-Phe-Asn-S8-3Thi-BztA-Gln-Ala-Ala | 594 |
| I-875 | Ac-PL3-Asp-Npg-B5-Asp-Asp-Ala-Ala-Phe-Asn-S8-3Thi-BztA-Gln-dAla-Ala | 595 |
| I-876 | Ac-PL3-Asp-Npg-B5-Asp-Asp-Ala-Ala-Phe-Asn-S8-3Thi-BztA-Gln-dAla-dAla | 596 |
| I-877 | Ac-PL3-Asp-Npg-B5-Asp-Asp-Ala-Ala-Phe-Asn-S8-3Thi-BztA-Gln-Ala-Ala | 594 |
| I-878 | Ac-PL3-Asp-Npg-B5-Asp-Asp-Ala-Ala-Phe-Asn-PyrS2-3Thi-BztA-[AzAc]Lys-NH2 | 597 |
| I-879 | Ac-PL3-Asp-Npg-B5-Asp-Asp-[AzAc]Lys-Ala-Phe-Asn-PyrS2-3Thi-BztA-Gln-NH2 | 598 |
| I-880 | Ac-PL3-Asp-[AzAc]Lys-B5-Asp-Asp-Ala-Ala-Phe-Asn-PyrS2-3Thi-BztA-Gln-NH2 | 599 |
| I-881 | Ac-PL3-Asp-Npg-B5-Asp-Asp-Ala-Ala-Phe-Asn-PyrS2-3Thi-BztA-Gln-6AmHex | 600 |
| I-882 | Ac-PL3-Asp-Npg-B5-Asp-Asp-Ala-Ala-Phe-Asn-PyrS2-3Thi-BztA-Gln-6AmHex | 600 |
| I-883 | Ac-PL3-Asp-Npg-B5-Asp-Asp-Ala-[35CF3PhPr]Lys-Phe-Asn-PyrS2-3Thi-BztA-Gln- | 601 |
| | NH2 | |
| I-884 | Ac-PL3-Asp-Npg-B5-Asp-Asp-Ala-[1NapPr]Lys-Phe-Asn-PyrS2-3Thi-BztA-Gln-NH2 | 602 |
| I-885 | Ac-PL3-Asp-Npg-B5-Asp-Asp-Ala-[22PhPr]Lys-Phe-Asn-PyrS2-3Thi-BztA-Gln-NH2 | 603 |
| I-886 | Ac-PL3-Asp-Npg-B5-Asp-Asp-Ala-[MorphAc]Lys-Phe-Asn-PyrS2-3Thi-BztA-Gln- | 604 |
| | NH2 | |
| I-887 | Ac-PL3-Asp-Npg-B5-Asp-Asp-Ala-[MePipAc]Lys-Phe-Asn-PyrS2-3Thi-BztA-Gln- | 605 |
| | NH2 | |
| I-888 | Ac-PL3-Asp-Npg-B5-Asp-Asp-Ala-[MeBipipAc]Lys-Phe-Asn-PyrS2-3Thi-BztA-Gln- | 606 |
| | NH2 | |
| I-889 | Ac-PL3-Asp-Npg-B5-Asp-Asp-Ala-[4MePipBz]Lys-Phe-Asn-PyrS2-3Thi-BztA-Gln- | 607 |
| | NH2 | |
| I-890 | Ac-PL3-Asp-Npg-B5-Asp-Asp-Ala-[MeMorphBz]Lys-Phe-Asn-PyrS2-3Thi-BztA-Gln- | 608 |
| | NH2 | |
| I-891 | Ac-PL3-Asp-Npg-B5-Asp-Asp-Ala-[Me2NCBz]Lys-Phe-Asn-PyrS2-3Thi-BztA-Gln- | 609 |
| | NH2 | |
| I-892 | Ac-PL3-Asp-Npg-B5-Asp-Asp-Ala-[mPEG2]Lys-Phe-Asn-PyrS2-3Thi-BztA-Gln-NH2 | 610 |
| I-893 | Ac-PL3-Asp-Npg-B5-Asp-Asp-Ala-[mPEG2]Lys-Phe-Asn-PyrS2-3Thi-BztA-Gln-NH2 | 610 |
| I-894 | Ac-PL3-Asp-Npg-B5-Asp-Asp-Ala-[mPEG4]Lys-Phe-Asn-PyrS2-3Thi-BztA-Gln-NH2 | 611 |
| I-895 | Ac-PL3-Asp-Npg-B5-Asp-Asp-Ala-[mPEG6]Lys-Phe-Asn-PyrS2-3Thi-BztA-Gln-NH2 | 612 |
| I-896 | Ac-PL3-Asp-Npg-B5-Asp-Asp-Ala-[mPEG8]Lys-Phe-Asn-PyrS2-3Thi-BztA-Gln-NH2 | 613 |
| I-897 | Ac-PL3-Asp-Npg-B5-Asp-Asp-Ala-[Bua]Lys-Phe-Asn-PyrS2-3Thi-BztA-Gln-NH2 | 614 |
| I-898 | Ac-PL3-Asp-Npg-B5-Asp-Asp-Ala-[Oct]Lys-Phe-Asn-PyrS2-3Thi-BztA-Gln-NH2 | 615 |
| I-899 | Ac-PL3-Asp-Npg-B5-Asp-Asp-Ala-[AdamantC]Lys-Phe-Asn-PyrS2-3Thi-BztA-Gln- | 616 |
| | NH2 | |
| I-900 | Ac-PL3-Asp-Npg-B5-Asp-Asp-Ala-[Me3AdamantC]Lys-Phe-Asn-PyrS2-3Thi-BztA- | 617 |
| | Gln-NH2 | |
| I-901 | Ac-PL3-Asp-Npg-B5-Asp-Asp-Ala-[AdamantPro]Lys-Phe-Asn-PyrS2-3Thi-BztA-Gln- | 618 |
| | NH2 | |
| I-902 | Ac-PL3-Asp-Npg-B5-Asp-Asp-Ala-Ala-Phe-Asn-PyrS2-3Thi-BztA-Gln- | 619 |
| | [35CF3PhPr]Lys-NH2 | |
| I-903 | Ac-PL3-Asp-Npg-B5-Asp-Asp-Ala-Ala-Phe-Asn-PyrS2-3Thi-BztA-Gln-[1NapPr]Lys- | 620 |
| | NH2 | |
| I-904 | Ac-PL3-Asp-Npg-B5-Asp-Asp-Ala-Ala-Phe-Asn-PyrS2-3Thi-BztA-Gln-[22PhPr]Lys- | 621 |
| | NH2 | |
| I-905 | Ac-PL3-Asp-Npg-B5-Asp-Asp-Ala-Ala-Phe-Asn-PyrS2-3Thi-BztA-Gln- | 622 |
| | [Morph Ac]Lys-NH2 | |
| I-906 | Ac-PL3-Asp-Npg-B5-Asp-Asp-Ala-Ala-Phe-Asn-PyrS2-3Thi-BztA-Gln- | 623 |
| | MePipAc Lys-NH2 | |
| I-907 | Ac-PL3-Asp-Npg-B5-Asp-Asp-Ala-Ala-Phe-Asn-PyrS2-3Thi-BztA-Gln- | 624 |
| | [MeBipipAc]Lys-NH2 | |
| I-908 | Ac-PL3-Asp-Npg-B5-Asp-Asp-Ala-Ala-Phe-Asn-PyrS2-3Thi-BztA-Gln- | 625 |
| | [4MePipBz]Lys-NH2 | |
| I-909 | Ac-PL3-Asp-Npg-B5-Asp-Asp-Ala-Ala-Phe-Asn-PyrS2-3Thi-BztA-Gln- | 626 |
| | [MeMorphBz]Lys-NH2 | |
| I-910 | Ac-PL3-Asp-Npg-B5-Asp-Asp-Ala-Ala-Phe-Asn-PyrS2-3Thi-BztA-Gln- | 627 |
| | [Me2NCBz]Lys-NH2 | |
| I-911 | Ac-PL3-Asp-Npg-B5-Asp-Asp-Ala-Ala-Phe-Asn-PyrS2-3Thi-BztA-Gln-[mPEG2]Lys- | 628 |
| | NH2 | |
| I-912 | Ac-PL3-Asp-Npg-B5-Asp-Asp-Ala-Ala-Phe-Asn-PyrS2-3Thi-BztA-Gln-[mPEG4]Lys- | 629 |
| | NH2 | |
| I-913 | Ac-PL3-Asp-Npg-B5-Asp-Asp-Ala-Ala-Phe-Asn-PyrS2-3Thi-BztA-Gln-[mPEG6]Lys- | 630 |
| | NH2 | |
| I-914 | Ac-PL3-Asp-Npg-B5-Asp-Asp-Ala-Ala-Phe-Asn-PyrS2-3Thi-BztA-Gln-[mPEG8]Lys- | 631 |
| | NH2 | |
| I-915 | Ac-PL3-Asp-Npg-B5-Asp-Asp-Ala-Ala-Phe-Asn-PyrS2-3Thi-BztA-Gln-[2Napc]Lys- | 632 |
| | NH2 | |
| I-916 | Ac-PL3-Asp-Npg-B5-Asp-Asp-Ala-Ala-Phe-Asn-PyrS2-3Thi-BztA-Gln-[1Napc]Lys- | 633 |
| | NH2 | |
| I-917 | Ac-PL3-Asp-Npg-B5-Asp-Asp-Ala-Ala-Phe-Asn-PyrS2-3Thi-BztA-Gln-[Bua]Lys-NH2 | 634 |
| I-918 | Ac-PL3-Asp-Npg-B5-Asp-Asp-Ala-Ala-Phe-Asn-PyrS2-3Thi-BztA-Gln-[Oct]Lys-NH2 | 635 |
| I-919 | Ac-PL3-Asp-Npg-B5-Asp-Asp-Ala-Ala-Phe-Asn-PyrS2-3Thi-BztA-Gln- | 636 |
| | [AdamantC]Lys-NH2 | |
| I-920 | Ac-PL3-Asp-Npg-B5-Asp-Asp-Ala-Ala-Phe-Asn-PyrS2-3Thi-BztA-Gln- | 637 |
| | [Me3AdamantC]Lys-NH2 | |
| I-921 | Ac-PL3-Asp-Npg-B5-Asp-TfeGA-Ala-Ala-Phe-Asn-PyrS2-2C1F-BztA-Gln-NH2 | 638 |
| I-922 | Ac-PL3-Asp-Npg-B5-Asp-TfeGA-Ala-Ala-Phe-Thr-PyrS2-2C1F-BztA-Gln-NH2 | 639 |
| I-923 | Ac-PL3-Asp-Npg-B5-Asp-TfeGA-Ala-Ala-Phe-Phe-PyrS2-2C1F-BztA-Gln-NH2 | 640 |
| I-924 | Ac-PL3-Asp-Npg-B5-Asp-TfeGA-Ala-Ala-Phe-Asn-PyrS2-2BrF-BztA-Gln-NH2 | 641 |
| I-925 | Ac-PL3-Asp-Npg-B5-Asp-TfeGA-Ala-Ala-Phe-Asn-PyrS2-3C1F-BztA-Gln-NH2 | 642 |
| I-926 | Ac-PL3-Asp-Npg-B5-Asp-TfeGA-Ala-Ala-Phe-Asn-PyrS2-3C1F-BztA-Gln-NH2 | 642 |
| I-927 | Ac-PL3-Asp-Npg-B5-Asp-TfeGA-Ala-Ala-Phe-Asn-PyrS2-2F3MeF-BztA-Gln-NH2 | 643 |
| I-928 | Ac-PL3-Asp-Npg-B5-Asp-TfeGA-Ala-Ala-Phe-Asn-PyrS2-2F3MeF-BztA-Gln-NH2 | 643 |
| I-929 | Ac-PL3-Asp-Npg-B5-Asp-TfeGA-Ala-Ala-Phe-Asn-S8-2C1F-BztA-Gln-NH2 | 644 |
| I-930 | Ac-PL3-Asp-Npg-B5-Asp-TfeGA-Ala-Ala-Phe-Thr-S8-2C1F-BztA-Gln-NH2 | 645 |
| I-931 | Ac-PL3-Asp-Npg-B5-Asp-TfeGA-Ala-Ala-Phe-Phe-S8-2C1F-BztA-Gln-NH2 | 646 |
| I-932 | Ac-PL3-Asp-Npg-B5-Asp-TfeGA-Ala-Ala-Phe-Asn-S8-2BrF-BztA-Gln-NH2 | 647 |
| I-933 | Ac-PL3-Asp-Npg-B5-Asp-TfeGA-Ala-Ala-Phe-Asn-S8-3C1F-BztA-Gln-NH2 | 648 |
| I-934 | Ac-PL3-Asp-Npg-B5-Asp-TfeGA-Ala-Ala-Phe-Asn-S8-2F3MeF-BztA-Gln-NH2 | 649 |
| I-935 | Ac-PL3-Asp-Npg-B5-Asp-Asp-Ala-Ala-Phe-Asn-PyrS2-3Thi-BztA-Gln- | 650 |
| | [PropynPEG1]Lys-NH2 | |
| I-936 | Ac-PL3-Asp-Npg-B5-Asp-Asp-Ala-Ala-Phe-Asn-PyrS2-3Thi-BztA-Gln- | 651 |
| | [PropynPEG2]Lys-NH2 | |
| I-937 | Ac-PL3-Asp-Npg-B5-Asp-Asp-Ala-Ala-Phe-Asn-PyrS2-3Thi-BztA-Gln- | 652 |
| | [PropynPEG3]Lys-NH2 | |
| I-938 | Ac-PL3-Asp-Npg-B5-Asp-Asp-Ala-Ala-Phe-Asn-PyrS2-3Thi-BztA-Gln- | 653 |
| | [PropynPEG4]Lys-NH2 | |
| I-939 | Ac-PL3-Asp-Npg-B5-Asp-Asp-Ala-Ala-Phe-Asn-PyrS2-3Thi-BztA-Gln-Lys-NH2 | 654 |
| I-940 | Ac-PL3-Asp-Npg-B5-Asp-TfeGA-Ala-Ala-Phe-Asn-PyrS2-3Thi-BztA-Ser-NH2 | 655 |
| I-941 | Ac-PL3-Asp-Npg-B5-Asp-TfeGA-Ala-Ala-Phe-Asn-PyrS2-3Thi-BztA-Ser-NH2 | 655 |
| I-942 | Ac-PL3-Asp-Npg-B5-Asp-TfeGA-Ala-Ala-Phe-Asn-PyrS2-3Thi-BztA-Thr-NH2 | 656 |
| I-943 | Ac-PL3-Asp-Npg-B5-Asp-TfeGA-Ala-Ala-Phe-Asn-PyrS2-3Thi-BztA-Thr-NH2 | 656 |
| I-944 | Ac-PL3-Asp-Npg-B5-Asp-TfeGA-Ala-Ala-Phe-Asn-PyrS2-3Thi-BztA-Ala-NH2 | 657 |
| I-945 | Ac-PL3-Asp-Npg-B5-Asp-TfeGA-Ala-Ala-Phe-Asn-PyrS2-3Thi-BztA-Ala-NH2 | 657 |
| I-946 | Ac-PL3-Asp-Npg-B5-Asp-TfeGA-Ala-Ala-Phe-Asn-PyrS2-3Thi-BztA-Leu-NH2 | 658 |
| I-947 | Ac-PL3-Asp-Npg-B5-Asp-TfeGA-Ala-Ala-Phe-Asn-PyrS2-3Thi-BztA-Leu-NH2 | 658 |
| I-948 | Ac-PL3-Asp-Npg-B5-Asp-TfeGA-Ala-Ala-Phe-Asn-PyrS2-3Thi-BztA-dThr-NH2 | 659 |
| I-949 | Ac-PL3-Asp-Npg-B5-Asp-TfeGA-Ala-Ala-Phe-Asn-PyrS2-3Thi-BztA-dThr-NH2 | 659 |
| I-950 | Ac-PL3-Asp-Npg-B5-Asp-TfeGA-Ala-Ala-Phe-Asn-PyrS2-3Thi-BztA-Hse-NH2 | 660 |
| I-951 | Ac-PL3-Asp-Npg-B5-Asp-TfeGA-Ala-Ala-Phe-Asn-PyrS2-3Thi-BztA-Hse-NH2 | 660 |
| I-952 | Ac-PL3-Asp-Npg-B5-Asp-TfeGA-Ala-Ala-Phe-Asn-PyrS2-3Thi-BztA-Tyr-NH2 | 661 |
| I-953 | Ac-PL3-Asp-Npg-B5-Asp-TfeGA-Ala-Ala-Phe-Asn-PyrS2-3Thi-BztA-Tyr-NH2 | 661 |
| I-954 | Ac-PL3-Asp-Npg-B5-Asp-TfeGA-Ala-Ala-Phe-Ser-PyrS2-3Thi-BztA-Ser-NH2 | 662 |
| I-955 | Ac-PL3-Asp-Npg-B5-Asp-TfeGA-Ala-Ala-Phe-Ser-PyrS2-3Thi-BztA-Ser-NH2 | 662 |
| I-956 | Ac-PL3-Asp-Npg-B5-Asp-TfeGA-Ala-Ala-Phe-Thr-PyrS2-3Thi-BztA-Ser-NH2 | 663 |
| I-957 | Ac-PL3-Asp-Npg-B5-Asp-TfeGA-Ala-Ala-Phe-Thr-PyrS2-3Thi-BztA-Ser-NH2 | 663 |
| I-958 | Ac-PL3-Asp-Npg-B5-Asp-TfeGA-Ala-Ala-Phe-Hse-PyrS2-3Thi-BztA-Ser-NH2 | 664 |
| I-959 | Ac-PL3-Asp-Npg-B5-Asp-TfeGA-Ala-Ala-Phe-Hse-PyrS2-3Thi-BztA-Ser-NH2 | 664 |
| I-960 | Ac-PL3-Asp-Npg-B5-Asp-TfeGA-Ala-Ala-Phe-Leu-PyrS2-3Thi-BztA-Ser-NH2 | 665 |
| I-961 | Ac-PL3-Asp-Npg-B5-Asp-TfeGA-Ala-Ala-Phe-Leu-PyrS2-3Thi-BztA-Ser-NH2 | 665 |
| I-962 | Ac-PL3-Asp-Npg-B5-Asp-TfeGA-Ala-Ala-Phe-Phe-PyrS2-3Thi-BztA-Ser-NH2 | 666 |
| I-963 | Ac-PL3-Asp-Npg-B5-Asp-TfeGA-Ala-Ala-Phe-Phe-PyrS2-3Thi-BztA-Ser-NH2 | 666 |
| I-964 | Ac-PL3-Asp-Npg-B5-Asp-EtGA-Ala-Ala-Phe-Asn-PyrS2-3Thi-BztA-Gln-NH2 | 667 |
| I-965 | Ac-PL3-Asp-Npg-B5-Asp-EtGA-Ala-Ala-Phe-Asn-PyrS2-3Thi-BztA-Gln-NH2 | 667 |
| I-966 | Ac-PL3-Asp-Npg-B5-Asp-EtGA-Ala-Ala-Phe-Ser-PyrS2-3Thi-BztA-Gln-NH2 | 668 |
| I-967 | Ac-PL3-Asp-Npg-B5-Asp-EtGA-Ala-Ala-Phe-Ser-PyrS2-3Thi-BztA-Gln-NH2 | 668 |
| I-968 | Ac-PL3-Asp-Npg-B5-Asp-EtGA-Ala-Ala-Phe-Thr-PyrS2-3Thi-BztA-Gln-NH2 | 669 |
| I-969 | Ac-PL3-Asp-Npg-B5-Asp-EtGA-Ala-Ala-Phe-Thr-PyrS2-3Thi-BztA-Gln-NH2 | 669 |
| I-970 | Ac-PL3-Asp-Npg-B5-Asp-EtGA-Ala-Ala-Phe-Phe-PyrS2-3Thi-BztA-Gln-NH2 | 670 |
| I-971 | Ac-PL3-Asp-Npg-B5-Asp-EtGA-Ala-Ala-Phe-Phe-PyrS2-3Thi-BztA-Gln-NH2 | 670 |
| I-972 | Ac-PL3-Asp-Npg-B5-Asp-EtGA-Ala-Ala-Phe-Leu-PyrS2-3Thi-BztA-Gln-NH2 | 671 |
| I-973 | Ac-PL3-Asp-Npg-B5-Asp-EtGA-Ala-Ala-Phe-Leu-PyrS2-3Thi-BztA-Gln-NH2 | 671 |
| I-974 | Ac-PL3-Asp-Npg-B5-Asp-EtGA-Ala-Ala-Phe-Ser-PyrS2-3Thi-BztA-Ser-NH2 | 672 |
| I-975 | Ac-PL3-Asp-Npg-B5-Asp-EtGA-Ala-Ala-Phe-Ser-PyrS2-3Thi-BztA-Ser-NH2 | 672 |
| I-976 | Ac-PL3-Asp-Npg-B5-Asp-3COOHF-Ala-Ala-Phe-Asn-PyrS2-3Thi-BztA-Gln-NH2 | 673 |
| I-977 | Ac-PL3-Asp-Npg-B5-Asp-3COOHF-Ala-Ala-Phe-Asn-PyrS2-3Thi-BztA-Gln-NH2 | 673 |
| I-978 | Ac-PL3-Asp-Npg-B5-Asp-3COOHF-Ala-Ala-Phe-Ser-PyrS2-3Thi-BztA-Gln-NH2 | 674 |
| I-979 | Ac-PL3-Asp-Npg-B5-Asp-3COOHF-Ala-Ala-Phe-Ser-PyrS2-3Thi-BztA-Gln-NH2 | 674 |
| I-980 | Ac-PL3-Asp-Npg-B5-Asp-3COOHF-Ala-Ala-Phe-Thr-PyrS2-3Thi-BztA-Gln-NH2 | 675 |
| I-981 | Ac-PL3-Asp-Npg-B5-Asp-3COOHF-Ala-Ala-Phe-Thr-PyrS2-3Thi-BztA-Gln-NH2 | 675 |
| I-982 | Ac-PL3-Asp-Npg-B5-Asp-3COOHF-Ala-Ala-Phe-Phe-PyrS2-3Thi-BztA-Ser-NH2 | 676 |
| I-983 | Ac-PL3-Asp-Npg-B5-Asp-3COOHF-Ala-Ala-Phe-Phe-PyrS2-3Thi-BztA-Ser-NH2 | 676 |
| I-984 | Ac-PL3-Asp-Npg-B5-Asp-3COOHF-Ala-Ala-Phe-Ser-PyrS2-3Thi-BztA-Ser-NH2 | 677 |
| I-985 | Ac-PL3-Asp-Npg-B5-Asp-3COOHF-Ala-Ala-Phe-Ser-PyrS2-3Thi-BztA-Ser-NH2 | 677 |
| I-986 | Ac-PL3-Asp-Npg-B5-Asp-3COOHF-Ala-Ala-Phe-Leu-PyrS2-3Thi-BztA-Ser-NH2 | 678 |
| I-987 | Ac-PL3-Asp-Npg-B5-Asp-3COOHF-Ala-Ala-Phe-Leu-PyrS2-3Thi-BztA-Ser-NH2 | 678 |
| I-988 | Ac-PL3-Asp-Npg-B5-Asp-TfeGA-Ala-Ala-Phe-Phe-PyrS2-Phe-BztA-Gln-NH2 | 679 |
| I-989 | Ac-PL3-Asp-Npg-B5-Asp-TfeGA-Ala-Ala-Phe-Phe-PyrS2-Tyr-BztA-Gln-NH2 | 680 |
| I-990 | Ac-PL3-Asp-Npg-B5-Asp-TfeGA-Ala-Ala-Phe-Phe-PyrS2-2C1F-BztA-Ser-NH2 | 681 |
| I-991 | Ac-PL3-Asp-Npg-B5-Asp-TfeGA-Ala-Ala-Phe-Phe-PyrS2-2C1F-BztA-Thr-NH2 | 682 |
| I-992 | Ac-PL3-Asp-Npg-B5-Asp-TfeGA-Ala-Ala-Phe-Phe-PyrS2-2C1F-BztA-Hse-NH2 | 683 |
| I-993 | Ac-PL3-Asp-Npg-B5-Asp-TfeGA-Ala-Ala-Phe-Hse-PyrS2-2C1F-BztA-Hse-NH2 | 684 |
| I-994 | Ac-PL3-Asp-Npg-B5-Asp-TfeGA-Ala-Ala-Phe-Leu-S8-2C1F-BztA-Gln-NH2 | 685 |
| I-995 | Ac-PL3-Asp-Npg-B5-Asp-TfeGA-Ala-Ala-Phe-Ser-S8-2C1F-BztA-Gln-NH2 | 686 |
| I-996 | Ac-PL3-Asp-Npg-B5-Asp-TfeGA-Ala-Ala-Phe-Hse-S8-2C1F-BztA-Gln-NH2 | 687 |
| I-997 | Ac-PL3-Asp-Npg-B5-Asp-TfeGA-Ala-Ala-Phe-Leu-S8-2C1F-BztA-Hse-NH2 | 688 |
| I-998 | Ac-PL3-Asp-Npg-B5-Asp-TfeGA-Ala-Ala-Phe-Ser-S8-2C1F-BztA-Hse-NH2 | 689 |
| I-999 | Ac-PL3-Asp-Npg-B5-Asp-TfeGA-Ala-Ala-Phe-Ser-S8-2C1F-BztA-Hse-NH2 | 689 |
| I-1000 | Ac-PL3-Asp-Npg-B5-Asp-TfeGA-Ala-Ala-Phe-Hse-S8-2C1F-BztA-Hse-NH2 | 690 |
| I-1001 | Ac-PL3-Asp-Npg-B5-3Thi-TfeGA-Ala-Ala-Phe-Asn-PyrS2-Asp-BztA-Gln-NH2 | 691 |
| I-1002 | Ac-PL3-Asp-Npg-B5-Asp-TfeGA-Ala-Leu-Phe-Asn-PyrS2-2C1F-BztA-Gln-NH2 | 692 |
| I-1003 | Ac-PL3-Asp-Npg-B5-Asp-TfeGA-Ala-Phe-Phe-Asn-PyrS2-2C1F-BztA-Gln-NH2 | 693 |
| I-1004 | Ac-PL3-Asp-Npg-B5-Asp-TfeGA-Ala-Ser-Phe-Asn-PyrS2-2C1F-BztA-Gln-NH2 | 694 |
| I-1005 | Ac-PL3-Asp-Npg-B5-Asp-TfeGA-Ala-Ile-Phe-Asn-PyrS2-2C1F-BztA-Gln-NH2 | 695 |
| I-1006 | Ac-PL3-Asp-Npg-B5-Asp-TfeGA-Ala-Lys-Phe-Asn-PyrS2-2C1F-BztA-Gln-NH2 | 696 |
| I-1007 | Ac-PL3-Asp-Npg-B5-Asp-TfeGA-Leu-Ala-Phe-Asn-PyrS2-2C1F-BztA-Gln-NH2 | 697 |
| I-1008 | Ac-PL3-Asp-Npg-B5-Asp-TfeGA-Phe-Ala-Phe-Asn-PyrS2-2C1F-BztA-Gln-NH2 | 698 |
| I-1009 | Ac-PL3-Asp-Npg-B5-Asp-TfeGA-Ser-Ala-Phe-Asn-PyrS2-2C1F-BztA-Gln-NH2 | 699 |
| I-1010 | Ac-PL3-Asp-Npg-B5-Asp-TfeGA-Ile-Ala-Phe-Asn-PyrS2-2C1F-BztA-Gln-NH2 | 700 |
| I-1011 | Ac-PL3-Asp-Npg-B5-Asp-TfeGA-Lys-Ala-Phe-Asn-PyrS2-2C1F-BztA-Gln-NH2 | 701 |
| I-1012 | Ac-PL3-Asp-Npg-B5-Asp-TfeGA-Ala-Ala-Phe-Phe-PyrS2-2C1F-BztA-Gln-6AmHex | 702 |
| I-1013 | Ac-PL3-Asp-Npg-B5-Asp-Asp-Ala-Ala-Phe-Thr-PyrS2-2C1F-BztA-Gln-NH2 | 703 |
| I-1014 | Ac-PL3-Asp-Npg-B5-Asp-Asn-Ala-Ala-Phe-Thr-PyrS2-2C1F-BztA-Gln-NH2 | 704 |
| I-1015 | Ac-PL3-Asp-Npg-B5-Asp-Asp-Ala-Ala-Phe-Thr-PyrS2-2C1F-BztA-Ser-NH2 | 705 |
| I-1016 | Ac-PL3-Asp-Npg-B5-Asp-Asn-Ala-Ala-Phe-Thr-PyrS2-2ClF-BztA-Ser-NH2 | 706 |
| I-1017 | Ac-PL3-Asp-Npg-B5-Asp-Asp-Ala-Ala-Phe-Ser-PyrS2-2C1F-BztA-Gln-NH2 | 707 |
| I-1018 | Ac-PL3-Asp-Npg-B5-Asp-Asn-Ala-Ala-Phe-Ser-PyrS2-2C1F-BztA-Gln-NH2 | 708 |
| I-1019 | Ac-PL3-Asp-Npg-B5-Asp-Asp-Ala-Ala-Phe-Ser-PyrS2-2C1F-BztA-Ser-NH2 | 709 |
| I-1020 | Ac-PL3-Asp-Npg-B5-Asp-Asn-Ala-Ala-Phe-Ser-PyrS2-2C1F-BztA-Ser-NH2 | 710 |
| I-1021 | Ac-PL3-Asp-Npg-B5-Asp-Asp-Ala-Ala-Phe-Thr-PyrS2-2FF-BztA-Gln-NH2 | 711 |
| I-1022 | Ac-PL3-Asp-Npg-B5-Asp-Asn-Ala-Ala-Phe-Thr-PyrS2-2FF-BztA-Gln-NH2 | 712 |
| I-1023 | Ac-PL3-Asp-Npg-B5-Asp-Asp-Ala-Ala-Phe-Thr-PyrS2-2FF-BztA-Ser-NH2 | 713 |
| I-1024 | Ac-PL3-Asp-Npg-B5-Asp-Asn-Ala-Ala-Phe-Thr-PyrS2-2FF-BztA-Ser-NH2 | 714 |
| I-1025 | Ac-PL3-Asp-Npg-B5-Asp-Asp-Ala-Ala-Phe-Asn-PyrS2-3Thi-BztA-Gln-OH | 715 |
| I-1026 | Ac-PL3-Asp-Npg-B5-Asp-TfeGA-Ala-Ala-Phe-Asn-PyrS2-3Thi-BztA-Gln-OH | 716 |
| I-1027 | Ac-PL3-Phe-Npg-B5-Asp-Asp-Ala-Ala-Asp-Asn-PyrS2-3Thi-BztA-Gln-OH | 717 |
| I-1028 | Ac-PL3-Asp-Npg-B5-Asp-Asp-Ala-Ala-Phe-Asn-PyrS2-2C1F-BztA-dThr-NH2 | 718 |
| I-1029 | Ac-PL3-Glu-Npg-B5-Asp-Asp-Ala-Ala-Phe-Asn-PyrS2-2C1F-BztA-dThr-NH2 | 719 |
| I-1030 | Ac-PL3-Asp-Npg-B5-Glu-Asp-Ala-Ala-Phe-Asn-PyrS2-2C1F-BztA-dThr-NH2 | 720 |
| I-1031 | Ac-PL3-Asp-Npg-B5-Asp-Glu-Ala-Ala-Phe-Asn-PyrS2-2C1F-BztA-dThr-NH2 | 721 |
| I-1032 | Ac-PL3-Asp-Npg-B5-Asp-Asp-Ala-Ala-Phe-Ser-PyrS2-2C1F-BztA-dThr-NH2 | 722 |
| I-1033 | Ac-PL3-Asp-Npg-B5-Asp-Asp-Ala-Ala-Phe-Thr-PyrS2-2ClF-BztA-dThr-NH2 | 723 |
| I-1034 | Ac-PL3-Asp-Npg-B5-Asp-Asp-Ala-Ala-Phe-Phe-PyrS2-2C1F-BztA-dThr-NH2 | 724 |
| I-1035 | Ac-PL3-Asp-Npg-B5-Asp-Asp-Ala-Ala-Phe-Leu-PyrS2-2C1F-BztA-dThr-NH2 | 725 |
| I-1036 | Ac-PL3-Asp-Npg-B5-Asp-Glu-Ala-Ala-Phe-Ser-PyrS2-2C1F-BztA-dThr-NH2 | 726 |
| I-1037 | Ac-PL3-Asp-Npg-B5-Asp-Glu-Ala-Ala-Phe-Thr-PyrS2-2C1F-BztA-dThr-NH2 | 727 |
| I-1038 | Ac-PL3-Asp-Npg-B5-Asp-Glu-Ala-Ala-Phe-Phe-PyrS2-2C1F-BztA-dThr-NH2 | 728 |
| I-1039 | Ac-PL3-Asp-Npg-B5-Asp-Glu-Ala-Ala-Phe-Leu-PyrS2-2C1F-BztA-dThr-NH2 | 729 |
| I-1040 | Ac-PL3-Asp-Cha-B5-Asp-Asp-Ala-Ala-Phe-Asn-PyrS2-2C1F-BztA-Gln-NH2 | 730 |
| I-1041 | Ac-PL3-Asp-Phe-B5-Asp-Asp-Ala-Ala-Phe-Asn-PyrS2-2C1F-BztA-Gln-NH2 | 731 |
| I-1042 | Ac-PL3-Asp-Cha-B5-Asp-Asp-Ala-Ala-Phe-Thr-PyrS2-2C1F-BztA-Gln-NH2 | 732 |
| I-1043 | Ac-PL3-Asp-Phe-B5-Asp-Asp-Ala-Ala-Phe-Thr-PyrS2-2C1F-BztA-Gln-NH2 | 733 |
| I-1044 | Ac-PL3-Asp-Cha-B5-Asp-Asp-Ala-Ala-Phe-Ser-PyrS2-2C1F-BztA-Gln-NH2 | 734 |
| I-1045 | Ac-PL3-Asp-Phe-B5-Asp-Asp-Ala-Ala-Phe-Ser-PyrS2-2C1F-BztA-Gln-NH2 | 735 |
| I-1046 | Ac-PL3-Asp-Cha-B5-Asp-Asp-Ala-Ala-Phe-Asn-PyrS2-2C1F-BztA-Ser-NH2 | 736 |
| I-1047 | Ac-PL3-Asp-Phe-B5-Asp-Asp-Ala-Ala-Phe-Asn-PyrS2-2C1F-BztA-Ser-NH2 | 737 |
| I-1048 | Ac-PL3-Asp-Cha-B5-Asp-Asp-Ala-Ala-Phe-Thr-PyrS2-2C1F-BztA-Ser-NH2 | 738 |
| I-1049 | Ac-PL3-Asp-Phe-B5-Asp-Asp-Ala-Ala-Phe-Thr-PyrS2-2C1F-BztA-Ser-NH2 | 739 |
| I-1050 | Ac-PL3-Asp-Cha-B5-Asp-Asp-Ala-Ala-Phe-Ser-PyrS2-2C1F-BztA-Ser-NH2 | 740 |
| I-1051 | Ac-PL3-Asp-Phe-B5-Asp-Asp-Ala-Ala-Phe-Ser-PyrS2-2C1F-BztA-Ser-NH2 | 741 |
| I-1052 | AzAc-PL3-Asp-Npg-B5-Asp-Asp-Ala-Ala-Phe-Asn-S8-3Thi-BztA-Gln-NH2 | 742 |
| I-1053 | AzAc-PL3-Asp-Npg-B5-Asp-Asp-Ala-Ala-Phe-Asn-S8-3Thi-BztA-Gln-NH2 | 742 |
| I-1054 | AzAc-PL3-Asp-Npg-B5-Asp-TfeGA-Ala-Ala-Phe-Asn-PyrS2-3Thi-BztA-Gln-NH2 | 743 |
| I-1055 | AzAc-PL3-Asp-Npg-B5-Asp-Asp-Ala-Ala-Phe-Asn-PyrS2-Phe-BztA-His-NH2 | 744 |
| I-1056 | AzAc-PL3-Asp-Npg-B5-3Thi-TfeGA-Ala-Ala-Phe-Asn-PyrS2-Asp-BztA-Gln-NH2 | 745 |
| I-1057 | AzAc-PL3-Asp-Npg-B5-3Thi-TfeGA-Ala-Ala-Phe-Asn-PyrS2-Asp-BztA-Gln-NH2 | 745 |
| I-1058 | AzAc-PL3-Asp-Npg-B5-Asp-Asp-Ala-Ala-Phe-Asn-PyrS2-3Thi-BztA-dThr-NH2 | 746 |
| I-1059 | AzAc-PL3-Asp-Npg-B5-Asp-TfeGA-Ala-Ala-Phe-Thr-PyrS2-2C1F-BztA-Gln-NH2 | 747 |
| I-1060 | AzAc-PL3-Asp-Npg-B5-Asp-Asp-Ala-Ala-Phe-Thr-PyrS2-2C1F-BztA-dThr-NH2 | 748 |
| I-1061 | Ac-PL3-Asp-Npg-B5-Asp-TfeGA-Ala-Ala-Phe-Thr-PyrS2-2C1F-BztA-Gln-6AmHex | 749 |
| I-1062 | Ac-PL3-Asp-Npg-B5-Asp-Asp-Ala-Ala-Phe-Leu-PyrS2-2C1F-BztA-Gln-NH2 | 750 |
| I-1063 | Ac-PL3-Asp-Npg-B5-Asp-Glu-Ala-Ala-Phe-Leu-PyrS2-2C1F-BztA-Gln-NH2 | 751 |
| I-1064 | Ac-PL3-Asp-Npg-B5-Asp-Asp-Ala-Ala-Phe-Leu-PyrS2-2C1F-BztA-Ser-NH2 | 752 |
| I-1065 | Ac-PL3-Asp-Npg-B5-Asp-Asp-Ala-Ala-Phe-Leu-PyrS2-2C1F-BztA-Ser-NH2 | 752 |
| I-1066 | Ac-PL3-Asp-Npg-B5-Asp-Glu-Ala-Ala-Phe-Leu-PyrS2-2C1F-BztA-Ser-NH2 | 753 |
| I-1067 | Ac-PL3-Asp-Npg-B5-Asp-Glu-Ala-Ala-Phe-Leu-PyrS2-2C1F-BztA-Ser-NH2 | 753 |
| I-1068 | Ac-PL3-Asp-Npg-B5-Asp-Asp-Ala-Ala-Phe-Leu-PyrS2-2C1F-BztA-Thr-NH2 | 754 |
| I-1069 | Ac-PL3-Asp-Npg-B5-Asp-Asp-Ala-Ala-Phe-Leu-PyrS2-2C1F-BztA-Thr-NH2 | 754 |
| I-1070 | Ac-PL3-Asp-Npg-B5-Asp-Glu-Ala-Ala-Phe-Leu-PyrS2-2C1F-BztA-Thr-NH2 | 755 |
| I-1071 | Ac-PL3-Asp-Npg-B5-Asp-Glu-Ala-Ala-Phe-Leu-PyrS2-2C1F-BztA-Thr-NH2 | 755 |
| I-1072 | Ac-PL3-Asp-Npg-B5-Asp-Asp-Ala-Ala-Phe-Leu-PyrS2-Phe-BztA-Gln-NH2 | 756 |
| I-1073 | Ac-PL3-Asp-Npg-B5-Asp-Glu-Ala-Ala-Phe-Leu-PyrS2-Phe-BztA-Gln-NH2 | 757 |
| I-1074 | Ac-PL3-Asp-Npg-B5-Asp-Glu-Ala-Ala-Phe-Leu-PyrS2-Phe-BztA-Gln-NH2 | 757 |
| I-1075 | Ac-PL3-Asp-Cha-B5-Asp-Asp-Ala-Ala-Phe-Leu-PyrS2-2C1F-BztA-Gln-NH2 | 758 |
| I-1076 | Ac-PL3-Asp-Cha-B5-Asp-Asp-Ala-Ala-Phe-Leu-PyrS2-2C1F-BztA-Gln-NH2 | 758 |
| I-1077 | Ac-PL3-Asp-Cha-B5-Asp-Glu-Ala-Ala-Phe-Leu-PyrS2-2C1F-BztA-Gln-NH2 | 759 |
| I-1078 | Ac-PL3-Asp-Cha-B5-Asp-Glu-Ala-Ala-Phe-Leu-PyrS2-2C1F-BztA-Gln-NH2 | 759 |
| I-1079 | Ac-PL3-Asp-Npg-B5-Asp-Asp-Ala-Ala-Phe-Asn-PyrS2-2C1F-BztA-Gln-NHEt | 760 |
| I-1080 | Ac-PL3-Asp-Npg-B5-Asp-Asp-Ala-Ala-Phe-Asn-PyrS2-2C1F-BztA-Leuo1 | 761 |
| I-1081 | Ac-PL3-Asp-Npg-B5-Asp-Asp-Ala-Ala-Phe-Asn-PyrS2-2C1F-BztA-Alao1 | 762 |
| I-1082 | Ac-PL3-Asp-Npg-B5-Asp-Asp-Ala-Ala-Phe-Asn-PyrS2-2C1F-BztA-Thro1 | 763 |
| I-1083 | Ac-PL3-Asp-Npg-B5-Asp-Asp-Ala-Ala-Phe-Asn-PyrS2-2C1F-BztA-Pheo1 | 764 |
| I-1084 | Ac-PL3-Asp-Npg-B5-Asp-Asp-Ala-Ala-Phe-Asn-PyrS2-2C1F-BztA-Proo1 | 765 |
| I-1085 | Ac-PL3-Asp-Npg-B5-Asp-TfeGA-Ala-Ala-Phe-Phe-PyrS2-2C1F-BztA-Gln-6AzHex | 766 |
| I-1086 | Ac-PL3-Asp-Npg-B5-Asp-Asp-Ala-Ala-Phe-Thr-S8-2C1F-BztA-Gln-NH2 | 767 |
| I-1087 | Ac-PL3-Asp-Npg-B5-Asp-Asp-Ala-Ala-Phe-Thr-S8-2C1F-BztA-Gln-NH2 | 767 |
| I-1088 | Ac-PL3-Asp-Npg-B5-Asp-Glu-Ala-Ala-Phe-Thr-S8-2ClF-BztA-Gln-NH2 | 768 |
| I-1089 | Ac-PL3-Asp-Npg-B5-Asp-Glu-Ala-Ala-Phe-Thr-S8-2C1F-BztA-Gln-NH2 | 768 |
| I-1090 | Ac-PL3-Asp-Npg-B5-Asp-Asp-Ala-Ala-Phe-Thr-S8-2C1F-BztA-Thr-NH2 | 769 |
| I-1091 | Ac-PL3-Asp-Npg-B5-Asp-Glu-Ala-Ala-Phe-Thr-S8-2C1F-BztA-Thr-NH2 | 770 |
| I-1092 | Ac-PL3-Asp-Npg-B5-Asp-Asp-Ala-Ala-Phe-Thr-S8-2C1F-BztA-dThr-NH2 | 771 |
| I-1093 | Ac-PL3-Asp-Npg-B5-Asp-Glu-Ala-Ala-Phe-Thr-S8-2C1F-BztA-dThr-NH2 | 772 |
| I-1094 | Ac-PL3-Asp-Npg-B5-Asp-Glu-Ala-Ala-Phe-Thr-S8-2C1F-BztA-dThr-NH2 | 772 |
| I-1095 | Ac-PL3-Asp-Npg-B5-Asp-Asp-Ala-Ala-Phe-Leu-S8-2C1F-BztA-Gln-NH2 | 773 |
| I-1096 | Ac-PL3-Asp-Npg-B5-Asp-Glu-Ala-Ala-Phe-Leu-S8-2C1F-BztA-Gln-NH2 | 774 |
| I-1097 | Ac-PL3-Asp-Npg-B5-Asp-Asp-Ala-Ala-Phe-Leu-S8-2C1F-BztA-dThr-NH2 | 775 |
| I-1098 | Ac-PL3-Asp-Npg-B5-Asp-Glu-Ala-Ala-Phe-Leu-S8-2ClF-BztA-dThr-NH2 | 776 |
| I-1099 | Ac-PL3-Asp-Npg-B5-Asp-Asp-Ala-Ala-Phe-Ser-PyrS2-Phe-BztA-Ser-NH2 | 777 |
| I-1100 | Ac-PL3-Asp-Cha-B5-Asp-Asp-Ala-Ala-Phe-Ser-PyrS2-Phe-BztA-Ser-NH2 | 778 |
| I-1101 | Ac-PL3-Asp-Npg-B5-Asp-Asp-Ala-Ala-Phe-Ser-PyrS2-2FF-BztA-Ser-NH2 | 779 |
| I-1102 | Ac-PL3-Asp-Cha-B5-Asp-Asp-Ala-Ala-Phe-Ser-PyrS2-2FF-BztA-Ser-NH2 | 780 |
| I-1103 | Ac-PL3-Asp-Npg-B5-Asp-Asp-Ala-Ala-Phe-Ser-PyrS2-2OMeF-BztA-Ser-NH2 | 781 |
| I-1104 | Ac-PL3-Asp-Cha-B5-Asp-Asp-Ala-Ala-Phe-Ser-PyrS2-2OMeF-BztA-Ser-NH2 | 782 |
| I-1105 | Ac-PL3-Asp-Npg-B5-Asp-Asp-Ala-Ala-Cha-Ser-PyrS2-Phe-BztA-Ser-NH2 | 783 |
| I-1106 | Ac-PL3-Asp-Npg-B5-Asp-Asp-Ala-Ala-Phe-Ser-PyrS2-Cha-BztA-Ser-NH2 | 784 |
| I-1107 | Ac-PL3-Asp-Cha-B5-Asp-Asp-Ala-Ala-Phe-Cha-PyrS2-2C1F-BztA-Gln-NH2 | 785 |
| I-1108 | Ac-PL3-Asp-1NapA-B5-Asp-Asp-Ala-Ala-Phe-Ser-PyrS2-2C1F-BztA-Gln-NH2 | 786 |
| I-1109 | Ac-PL3-Asp-2NapA-B5-Asp-Asp-Ala-Ala-Phe-Ser-PyrS2-2C1F-BztA-Gln-NH2 | 787 |
| I-1110 | Ac-PL3-Asp-1NapA-B5-Asp-Asp-Ala-Ala-Phe-Leu-PyrS2-2C1F-BztA-Gln-NH2 | 788 |
| I-1111 | Ac-PL3-Asp-2NapA-B5-Asp-Asp-Ala-Ala-Phe-Leu-PyrS2-2C1F-BztA-Gln-NH2 | 789 |
| I-1112 | Ac-PL3-Asp-Cha-B5-Asp-Asp-Ala-Ala-Phe-Leu-PyrS2-2C1F-BztA-Ser-NH2 | 790 |
| I-1113 | Ac-PL3-Asp-Cha-B5-Asp-Asp-Ala-Ala-Phe-Cha-PyrS2-2C1F-BztA-Ser-NH2 | 791 |
| I-1114 | Ac-PL3-Asp-1NapA-B5-Asp-Asp-Ala-Ala-Phe-Thr-PyrS2-2C1F-BztA-Ser-NH2 | 792 |
| I-1115 | Ac-PL3-Asp-2NapA-B5-Asp-Asp-Ala-Ala-Phe-Thr-PyrS2-2C1F-BztA-Ser-NH2 | 793 |
| I-1116 | Ac-PL3-Asp-1NapA-B5-Asp-Asp-Ala-Ala-Phe-Leu-PyrS2-2C1F-BztA-Ser-NH2 | 794 |
| I-1117 | Ac-PL3-Asp-2NapA-B5-Asp-Asp-Ala-Ala-Phe-Leu-PyrS2-2C1F-BztA-Ser-NH2 | 795 |
| I-1118 | Ac-PL3-Asp-Cha-B5-Asp-Asp-Ala-Ala-3OMeF-Ser-PyrS2-2C1F-BztA-Gln-NH2 | 796 |
| I-1119 | Ac-PL3-Asp-Cha-B5-Asp-Asp-Ala-Ala-3F3MeF-Ser-PyrS2-2C1F-BztA-Gln-NH2 | 797 |
| I-1120 | Ac-PL3-Asp-Cha-B5-Asp-Asp-Ala-Ala-4F3MeF-Ser-PyrS2-2C1F-BztA-Gln-NH2 | 798 |
| I-1121 | Ac-PL3-Asp-Npg-B5-Asp-Asp-Ala-Ala-3OMeF-Leu-PyrS2-2C1F-BztA-Gln-NH2 | 799 |
| I-1122 | Ac-PL3-Asp-Npg-B5-Asp-Asp-Ala-Ala-3F3MeF-Leu-PyrS2-2C1F-BztA-Gln-NH2 | 800 |
| I-1123 | Ac-PL3-Asp-Npg-B5-Asp-Asp-Ala-Ala-4F3MeF-Leu-PyrS2-2C1F-BztA-Gln-NH2 | 801 |
| I-1124 | Ac-PL3-Asp-Cha-B5-Asp-Asp-Ala-Ala-3OMeF-Ser-PyrS2-Phe-BztA-Gln-NH2 | 802 |
| I-1125 | Ac-PL3-Asp-Cha-B5-Asp-Asp-Ala-Ala-3F3MeF-Ser-PyrS2-Phe-BztA-Gln-NH2 | 803 |
| I-1126 | Ac-PL3-Asp-Cha-B5-Asp-Asp-Ala-Ala-4F3MeF-Ser-PyrS2-Phe-BztA-Gln-NH2 | 804 |
| I-1127 | Ac-PL3-Asp-Npg-B5-Asp-Asp-Ala-Ala-3OMeF-Leu-PyrS2-Phe-BztA-Gln-NH2 | 805 |
| I-1128 | Ac-PL3-Asp-Npg-B5-Asp-Asp-Ala-Ala-3F3MeF-Leu-PyrS2-Phe-BztA-Gln-NH2 | 806 |
| I-1129 | Ac-PL3-Asp-Npg-B5-Asp-Asp-Ala-Ala-4F3MeF-Leu-PyrS2-Phe-BztA-Gln-NH2 | 807 |
| I-1130 | Ac-PL3-Asp-Npg-B5-Asp-Asp-Ala-Ala-Phe-Asn-PyrS2-2C1F-BztA-iPrLys-NH2 | 808 |
| I-1131 | Ac-PL3-Asp-Npg-B5-Asp-Asp-iPrLys-Ala-Phe-Asn-PyrS2-2C1F-BztA-Gln-NH2 | 809 |
| I-1132 | Ac-PL3-Asp-Npg-B5-Asp-Asp-Ala-iPrLys-Phe-Asn-PyrS2-2C1F-BztA-Gln-NH2 | 810 |
| I-1133 | Ac-PL3-Asp-Npg-B5-Asp-Asp-Ala-Ala-Phe-Asn-PyrS2-2C1F-BztA-MorphAla-NH2 | 811 |
| I-1134 | Ac-PL3-Asp-Npg-B5-Asp-Asp-Ala-Ala-Phe-Asn-PyrS2-2C1F-BztA-MorphAla-NH2 | 811 |
| I-1135 | Ac-PL3-Asp-Cha-B5-Asp-Asp-Ala-Ala-Phe-Ser-PyrS2-2C1F-BztA-Thr-NH2 | 812 |
| I-1136 | Ac-PL3-Asp-Cha-B5-Asp-Asp-Ala-Ala-Phe-Ser-PyrS2-2C1F-BztA-dThr-NH2 | 813 |
| I-1137 | Ac-PL3-Asp-Cha-B5-Asp-Asp-Ala-Ala-Phe-Ser-PyrS2-2C1F-BztA-Hse-NH2 | 814 |
| I-1138 | Ac-PL3-Asp-Cha-B5-Asp-Asp-Ala-Ala-Phe-Ser-PyrS2-2C1F-BztA-Leu-NH2 | 815 |
| I-1139 | Ac-PL3-Asp-Cha-B5-Asp-Asp-Ala-Ala-Phe-Leu-PyrS2-2C1F-BztA-Thr-NH2 | 816 |
| I-1140 | Ac-PL3-Asp-Cha-B5-Asp-Asp-Ala-Ala-Phe-Leu-PyrS2-2C1F-BztA-dThr-NH2 | 817 |
| I-1141 | Ac-PL3-Asp-Phe-B5-Asp-Asp-Ala-Ala-Phe-Ser-PyrS2-2C1F-BztA-Thr-NH2 | 818 |
| I-1142 | Ac-PL3-Asp-Phe-B5-Asp-Asp-Ala-Ala-Phe-Ser-PyrS2-2C1F-BztA-dThr-NH2 | 819 |
| I-1143 | Ac-PL3-Asp-Phe-B5-Asp-Asp-Ala-Ala-Phe-Ser-PyrS2-2C1F-BztA-Hse-NH2 | 820 |
| I-1144 | Ac-PL3-Asp-Phe-B5-Asp-Asp-Ala-Ala-Phe-Ser-PyrS2-2C1F-BztA-Leu-NH2 | 821 |
| I-1145 | Ac-PL3-Asp-Phe-B5-Asp-Asp-Ala-Ala-Phe-Leu-PyrS2-2C1F-BztA-Thr-NH2 | 822 |
| I-1146 | Ac-PL3-Asp-Phe-B5-Asp-Asp-Ala-Ala-Phe-Leu-PyrS2-2C1F-BztA-dThr-NH2 | 823 |
| I-1147 | Ac-PL3-Asp-Cha-B5-Asp-Asp-Leu-Ala-Phe-Ser-PyrS2-2C1F-BztA-Gln-NH2 | 824 |
| I-1148 | Ac-PL3-Asp-Cha-B5-Asp-Asp-Ala-Leu-Phe-Ser-PyrS2-2C1F-BztA-Gln-NH2 | 825 |
| I-1149 | Ac-PL3-Asp-Cha-B5-Asp-Asp-Leu-Leu-Phe-Ser-PyrS2-2C1F-BztA-Gln-NH2 | 826 |
| I-1150 | Ac-PL3-Asp-Cha-B5-Asp-Asp-Leu-Ala-Phe-Thr-PyrS2-2C1F-BztA-Ser-NH2 | 827 |
| I-1151 | Ac-PL3-Asp-Cha-B5-Asp-Asp-Ala-Leu-Phe-Thr-PyrS2-2C1F-BztA-Ser-NH2 | 828 |
| I-1152 | Ac-PL3-Asp-Cha-B5-Asp-Asp-Leu-Leu-Phe-Thr-PyrS2-2C1F-BztA-Ser-NH2 | 829 |
| I-1153 | Ac-PL3-Asp-Phe-B5-Asp-Asp-Leu-Ala-Phe-Ser-PyrS2-2C1F-BztA-Gln-NH2 | 830 |
| I-1154 | Ac-PL3-Asp-Phe-B5-Asp-Asp-Ala-Leu-Phe-Ser-PyrS2-2C1F-BztA-Gln-NH2 | 831 |
| I-1155 | Ac-PL3-Asp-Phe-B5-Asp-Asp-Leu-Leu-Phe-Ser-PyrS2-2C1F-BztA-Gln-NH2 | 832 |
| I-1156 | Ac-PL3-Asp-Phe-B5-Asp-Asp-Leu-Ala-Phe-Thr-PyrS2-2C1F-BztA-Ser-NH2 | 833 |
| I-1157 | Ac-PL3-Asp-Phe-B5-Asp-Asp-Ala-Leu-Phe-Thr-PyrS2-2C1F-BztA-Ser-NH2 | 834 |
| I-1158 | Ac-PL3-Asp-Phe-B5-Asp-Asp-Leu-Leu-Phe-Thr-PyrS2-2C1F-BztA-Ser-NH2 | 835 |
| I-1159 | Ac-PL3-RbGlu-Npg-B5-Asp-Asp-Ala-Ala-Phe-Asn-PyrS2-3Thi-BztA-Gln-NH2 | 836 |
| I-1160 | Ac-PL3-NMeD-Npg-B5-Asp-Asp-Ala-Ala-Phe-Asn-PyrS2-3Thi-BztA-Gln-NH2 | 837 |
| I-1161 | Ac-PL3-RbGlu-Npg-B5-Asp-TfeGA-Ala-Ala-Phe-Asn-PyrS2-3Thi-BztA-Gln-NH2 | 838 |
| I-1162 | Ac-PL3-NMeD-Npg-B5-Asp-TfeGA-Ala-Ala-Phe-Asn-PyrS2-3Thi-BztA-Gln-NH2 | 839 |
| I-1163 | Ac-PL3-RbGlu-Npg-B5-Asp-Asp-Ala-Ala-Phe-Asn-S8-3Thi-BztA-Gln-NH2 | 840 |
| I-1164 | Ac-PL3-RbGlu-Npg-B5-Asp-Asp-Ala-Ala-Phe-Asn-S8-3Thi-BztA-Gln-NH2 | 840 |
| I-1165 | Ac-PL3-NMeD-Npg-B5-Asp-Asp-Ala-Ala-Phe-Asn-S8-3Thi-BztA-Gln-NH2 | 841 |
| I-1166 | Ac-PL3-Asp-Npg-B5-Asp-Thr-Ala-Ala-Phe-Thr-PyrS2-3Thi-BztA-Gln-NH2 | 842 |
| I-1167 | Ac-PL3-Asp-Npg-B5-Asp-Thr-Ala-Ala-Phe-Leu-PyrS2-3Thi-BztA-Gln-NH2 | 843 |
| I-1168 | Ac-PL3-Asp-Npg-B5-Asp-Thr-Ala-Ala-Phe-Thr-PyrS2-2C1F-BztA-Gln-NH2 | 844 |
| I-1169 | Ac-PL3-Asp-Npg-B5-Asp-Thr-Ala-Ala-Phe-Leu-PyrS2-2C1F-BztA-Gln-NH2 | 845 |
| I-1170 | Ac-PL3-Asp-Npg-B5-Asp-Thr-Ala-Ala-Phe-Thr-PyrS2-3Thi-BztA-Ser-NH2 | 846 |
| I-1171 | Ac-PL3-Asp-Npg-B5-Asp-Thr-Ala-Ala-Phe-Leu-PyrS2-3Thi-BztA-Ser-NH2 | 847 |
| I-1172 | Ac-PL3-Asp-Npg-B5-Asp-Thr-Ala-Ala-Phe-Leu-PyrS2-2C1F-BztA-Ser-NH2 | 848 |
| I-1173 | Ac-PL3-Asp-Cha-B5-Asp-Thr-Ala-Ala-Phe-Thr-PyrS2-3Thi-BztA-Gln-NH2 | 849 |
| I-1174 | Ac-PL3-Asp-Cha-B5-Asp-Thr-Ala-Ala-Phe-Leu-PyrS2-3Thi-BztA-Gln-NH2 | 850 |
| I-1175 | Ac-PL3-Asp-Cha-B5-Asp-Thr-Ala-Ala-Phe-Thr-PyrS2-2C1F-BztA-Gln-NH2 | 851 |
| I-1176 | Ac-PL3-Asp-Cha-B5-Asp-Thr-Ala-Ala-Phe-Leu-PyrS2-2C1F-BztA-Gln-NH2 | 852 |
| I-1177 | Ac-PL3-Asp-Npg-B5-Asp-Asp-Aib-Ala-Phe-Leu-PyrS2-3Thi-BztA-Gln-NH2 | 853 |
| I-1178 | Ac-PL3-Asp-Npg-B5-Asp-Asp-Ala-Aib-Phe-Leu-PyrS2-3Thi-BztA-Gln-NH2 | 854 |
| I-1179 | Ac-PL3-Asp-Npg-B5-Asp-Asp-Aib-Aib-Phe-Leu-PyrS2-3Thi-BztA-Gln-NH2 | 855 |
| I-1180 | Ac-PL3-Asp-Npg-B5-Asp-Asp-Aib-Aib-Phe-Leu-PyrS2-3Thi-BztA-Gln-NH2 | 855 |
| I-1181 | Ac-Pro-Asp-Npg-R5-Asp-Asp-Ala-Ala-Phe-Leu-PyrS2-3Thi-BztA-Gln-NH2 | 856 |
| I-1182 | Ac-Pro-Asp-Npg-R5-Asp-Asp-Ala-Ala-Phe-Asn-PyrS2-3Thi-BztA-Gln-NH2 | 857 |
| I-1183 | Ac-Pro-Asp-Npg-R5-Asp-Asp-Ala-Ala-Phe-Asn-PyrS2-2C1F-BztA-Gln-NH2 | 858 |
| I-1184 | Ac-Pro-Asp-Cha-R5-Asp-Asp-Ala-Ala-Phe-Thr-PyrS2-2C1F-BztA-Ser-NH2 | 859 |
| I-1185 | Ac-PL3-Asp-Npg-B5-Asp-Asp-Ala-Ala-Phe-Leu-PyrS2-Pff-BztA-Gln-NH2 | 860 |
| I-1186 | Ac-PL3-Asp-Npg-B5-Asp-Asp-Ala-Ala-Phe-Asn-PyrS2-Pff-BztA-Gln-NH2 | 861 |
| I-1187 | Ac-PL3-Asp-Cha-B5-Asp-Asp-Ala-Ala-Phe-Thr-PyrS2-Pff-BztA-Ser-NH2 | 862 |
| I-1188 | Ac-PL3-Asp-Npg-B5-Asp-Asp-Ala-Ala-Phe-Leu-S8-3Thi-BztA-Gln-NH2 | 863 |
| I-1189 | Ac-PL3-Asp-Npg-B5-Asp-Asp-Ala-Ala-Phe-Leu-S8-2C1F-BztA-Ser-NH2 | 864 |
| I-1190 | Ac-PL3-Asp-Npg-B5-Asp-Asp-Ala-Ala-Phe-Leu-S8-3Thi-BztA-Ser-NH2 | 865 |
| I-1191 | Ac-PL3-Asp-Npg-B5-Asp-Asp-Ala-Ala-Phe-Leu-S8-3Thi-BztA-Thr-NH2 | 866 |
| I-1192 | Ac-PL3-Asp-Npg-B5-Asp-Asp-Ala-Ala-Phe-Leu-S8-2C1F-BztA-4TriA-NH2 | 867 |
| I-1193 | Ac-PL3-Asp-Npg-B5-Asp-Asp-Ala-Ala-Phe-Leu-S8-3Thi-BztA-4TriA-NH2 | 868 |
| I-1194 | Ac-PL3-Asp-Npg-B5-Asp-Asp-Ala-Ala-Phe-Leu-S8-2C1F-BztA-His-NH2 | 869 |
| I-1195 | Ac-PL3-Asp-Npg-B5-Asp-Asp-Ala-Ala-Phe-Leu-S8-3Thi-BztA-His-NH2 | 870 |
| I-1196 | Ac-PL3-Asp-Npg-B5-Asp-Asp-Ala-Ala-Phe-Leu-S8-2C1F-BztA-Hse-NH2 | 871 |
| I-1197 | Ac-PL3-Asp-Npg-B5-Asp-Asp-Ala-Ala-Phe-Leu-S8-3Thi-BztA-Hse-NH2 | 872 |
| I-1198 | Ac-PL3-Asp-Npg-B5-Asp-Asp-Ala-Ala-Phe-Leu-S8-2C1F-BztA-dGln-NH2 | 873 |
| I-1199 | Ac-PL3-Asp-Npg-B5-Asp-Asp-Ala-Ala-Phe-Leu-S8-3Thi-BztA-dGln-NH2 | 874 |
| I-1200 | Ac-PL3-Asp-Npg-B5-Asp-Asp-Ala-Ala-2CNF-Leu-PyrS2-3Thi-BztA-Gln-NH2 | 875 |
| I-1201 | Ac-PL3-Asp-Npg-B5-Asp-Asp-Ala-Ala-3CNF-Leu-PyrS2-3Thi-BztA-Gln-NH2 | 876 |
| I-1202 | Ac-PL3-Asp-Npg-B5-Asp-Asp-Ala-Ala-4CNF-Leu-PyrS2-3Thi-BztA-Gln-NH2 | 877 |
| I-1203 | Ac-PL3-Asp-Npg-B5-Asp-Asp-Ala-Ala-Phe-Leu-PyrS2-2CNF-BztA-Gln-NH2 | 878 |
| I-1204 | Ac-PL3-Asp-Npg-B5-Asp-Asp-Ala-Ala-Phe-Leu-PyrS2-3CNF-BztA-Gln-NH2 | 879 |
| I-1205 | Ac-PL3-Asp-Npg-B5-Asp-Asp-Ala-Ala-Phe-Leu-PyrS2-4CNF-BztA-Gln-NH2 | 880 |
| I-1206 | Ac-PL3-Asp-Npg-B5-Asn-Asp-Ala-Ala-Phe-Asn-PyrS2-3Thi-BztA-Gln-NH2 | 881 |
| I-1207 | Ac-PL3-Asp-Npg-B5-Asn-Asp-Ala-Ala-Phe-Leu-PyrS2-3Thi-BztA-Gln-NH2 | 882 |
| I-1208 | Ac-PL3-Asp-Npg-B5-Ser-Asp-Ala-Ala-Phe-Asn-PyrS2-3Thi-BztA-Gln-NH2 | 883 |
| I-1209 | Ac-PL3-Asp-Npg-B5-Ser-Asp-Ala-Ala-Phe-Leu-PyrS2-3Thi-BztA-Gln-NH2 | 884 |
| I-1210 | Ac-PL3-Asp-Npg-B5-Gln-Asp-Ala-Ala-Phe-Asn-PyrS2-3Thi-BztA-Gln-NH2 | 885 |
| I-1211 | Ac-PL3-Asp-Npg-B5-Gln-Asp-Ala-Ala-Phe-Leu-PyrS2-3Thi-BztA-Gln-NH2 | 886 |
| I-1212 | Ac-PL3-Asp-Npg-B5-Leu-Asp-Ala-Ala-Phe-Asn-PyrS2-3Thi-BztA-Gln-NH2 | 887 |
| I-1213 | Ac-PL3-Asp-Npg-B5-Leu-Asp-Ala-Ala-Phe-Leu-PyrS2-3Thi-BztA-Gln-NH2 | 888 |
| I-1214 | Ac-PL3-Asp-Npg-B5-Hse-Asp-Ala-Ala-Phe-Asn-PyrS2-3Thi-BztA-Gln-NH2 | 889 |
| I-1215 | Ac-PL3-Asp-Npg-B5-Hse-Asp-Ala-Ala-Phe-Leu-PyrS2-3Thi-BztA-Gln-NH2 | 890 |
| I-1216 | Ac-PL3-Asp-Npg-B5-Dab-Asp-Ala-Ala-Phe-Asn-PyrS2-3Thi-BztA-Gln-NH2 | 891 |
| I-1217 | Ac-PL3-Asp-Npg-B5-Dab-Asp-Ala-Ala-Phe-Leu-PyrS2-3Thi-BztA-Gln-NH2 | 892 |
| I-1218 | MeSO2-PL3-Asp-Npg-B5-Asp-Asp-Ala-Ala-Phe-Asn-PyrS2-3Thi-BztA-Gln-NH2 | 893 |
| I-1219 | MeSO2-PL3-Asp-Npg-B5-Asp-TfeGA-Ala-Ala-Phe-Asn-PyrS2-3Thi-BztA-Gln-NH2 | 894 |
| I-1220 | Ac-PL3-Asn-Npg-B5-Asp-Asp-Ala-Ala-Phe-Asn-PyrS2-3Thi-BztA-Gln-NH2 | 895 |
| I-1221 | Ac-PL3-Asn-Npg-B5-Asp-Asp-Ala-Ala-Phe-Leu-PyrS2-3Thi-BztA-Gln-NH2 | 896 |
| I-1222 | Ac-PL3-Ser-Npg-B5-Asp-Asp-Ala-Ala-Phe-Asn-PyrS2-3Thi-BztA-Gln-NH2 | 897 |
| I-1223 | Ac-PL3-Ser-Npg-B5-Asp-Asp-Ala-Ala-Phe-Leu-PyrS2-3Thi-BztA-Gln-NH2 | 898 |
| I-1224 | Ac-PL3-Gln-Npg-B5-Asp-Asp-Ala-Ala-Phe-Asn-PyrS2-3Thi-BztA-Gln-NH2 | 899 |
| I-1225 | Ac-PL3-Gln-Npg-B5-Asp-Asp-Ala-Ala-Phe-Leu-PyrS2-3Thi-BztA-Gln-NH2 | 900 |
| I-1226 | Ac-PL3-Leu-Npg-B5-Asp-Asp-Ala-Ala-Phe-Asn-PyrS2-3Thi-BztA-Gln-NH2 | 901 |
| I-1227 | Ac-PL3-Leu-Npg-B5-Asp-Asp-Ala-Ala-Phe-Leu-PyrS2-3Thi-BztA-Gln-NH2 | 902 |
| I-1228 | Ac-PL3-Hse-Npg-B5-Asp-Asp-Ala-Ala-Phe-Asn-PyrS2-3Thi-BztA-Gln-NH2 | 903 |
| I-1229 | Ac-PL3-Hse-Npg-B5-Asp-Asp-Ala-Ala-Phe-Leu-PyrS2-3Thi-BztA-Gln-NH2 | 904 |
| I-1230 | Ac-PL3-Dab-Npg-B5-Asp-Asp-Ala-Ala-Phe-Asn-PyrS2-3Thi-BztA-Gln-NH2 | 905 |
| I-1231 | Ac-PL3-Dab-Npg-B5-Asp-Asp-Ala-Ala-Phe-Leu-PyrS2-3Thi-BztA-Gln-NH2 | 906 |
| I-1232 | Ac-PL3-Asp-Npg-B5-Asp-Asp-Ala-Ala-Phe-Thr-PyrS2-3Thi-1NapA-Gln-NH2 | 907 |
| I-1233 | Ac-PL3-Asp-Npg-B5-Asp-Asp-Ala-Ala-Phe-Thr-PyrS2-3Thi-2NapA-Gln-NH2 | 908 |
| I-1234 | Ac-PL3-Asp-Npg-B5-Asp-Asp-Ala-Ala-Phe-Leu-PyrS2-3Thi-1NapA-Gln-NH2 | 909 |
| I-1235 | Ac-PL3-Asp-Npg-B5-Asp-Asp-Ala-Ala-Phe-Leu-PyrS2-3Thi-2NapA-Gln-NH2 | 910 |
| I-1236 | Ac-PL3-Asp-Npg-B5-Asp-Asp-Ala-Ala-Phe-Leu-PyrS2-2C1F-1NapA-Gln-NH2 | 911 |
| I-1237 | Ac-PL3-Asp-Npg-B5-Asp-Asp-Ala-Ala-Phe-Leu-PyrS2-2C1F-2NapA-Gln-NH2 | 912 |
| I-1238 | Ac-PL3-Asp-Npg-B5-Asp-Asp-Ala-Ala-Phe-Leu-PyrS2-3Thi-1NapA-Ser-NH2 | 913 |
| I-1239 | Ac-PL3-Asp-Npg-B5-Asp-Asp-Ala-Ala-Phe-Leu-PyrS2-3Thi-2NapA-Ser-NH2 | 914 |
| I-1240 | Ac-PL3-Asp-Npg-B5-Asp-TfeGA-Ala-Ala-Phe-Thr-PyrS2-2C1F-BztA-Gln-NHEt | 915 |
| I-1241 | Ac-PL3-Asp-Npg-B5-Asp-TfeGA-Ala-Ala-Phe-Thr-PyrS2-2C1F-BztA-Thro1 | 916 |
| I-1242 | Ac-PL3-Asp-Npg-B5-Asp-TfeGA-Ala-Ala-Phe-Thr-PyrS2-2C1F-BztA-Alao1 | 917 |
| I-1243 | Ac-PL3-Asp-Npg-B5-Asp-TfeGA-Ala-Ala-Phe-Thr-PyrS2-2C1F-BztA-Leuo1 | 918 |
| I-1244 | Ac-PL3-Asp-Npg-B5-Asp-Asp-Ala-Ala-Phe-Thr-PyrS2-2C1F-BztA-Ser-NHEt | 919 |
| I-1245 | Ac-PL3-Asp-Npg-B5-Asp-Asp-Ala-Ala-Phe-Thr-PyrS2-2C1F-BztA-Thro1 | 920 |
| I-1246 | Ac-PL3-Asp-Npg-B5-Asp-Asp-Ala-Ala-Phe-Thr-PyrS2-2C1F-BztA-Alao1 | 921 |
| I-1247 | Ac-PL3-Asp-Npg-B5-Asp-Asp-Ala-Ala-Phe-Thr-PyrS2-2C1F-BztA-Leuo1 | 922 |
| I-1248 | Ac-PL3-Asp-Npg-B5-Asp-Asp-Ala-Ala-Phe-Leu-PyrS2-2C1F-BztA-Gln-NHEt | 923 |
| I-1249 | Ac-PL3-Asp-Npg-B5-Asp-Asp-Ala-Ala-Phe-Leu-PyrS2-2C1F-BztA-Thro1 | 924 |
| I-1250 | Ac-PL3-Asp-Npg-B5-Asp-Asp-Ala-Ala-Phe-Leu-PyrS2-2C1F-BztA-Alao1 | 925 |
| I-1251 | Ac-PL3-Asp-Npg-B5-Asp-Asp-Ala-Ala-Phe-Leu-PyrS2-2C1F-BztA-Leuo1 | 926 |
| I-1252 | Oct-PL3-Asp-Npg-B5-Asp-Asp-Ala-Ala-Phe-Asn-PyrS2-2C1F-BztA-dThr-NH2 | 927 |
| I-1253 | mPEG2-PL3-Asp-Npg-B5-Asp-Asp-Ala-Ala-Phe-Asn-PyrS2-2C1F-BztA-dThr-NH2 | 928 |
| I-1254 | Oct-PL3-Asp-Npg-B5-Asp-Asp-Ala-Ala-Phe-Thr-S8-2C1F-BztA-Gln-NH2 | 929 |
| I-1255 | mPEG2-PL3-Asp-Npg-B5-Asp-Asp-Ala-Ala-Phe-Thr-S8-2C1F-BztA-Gln-NH2 | 930 |
| I-1256 | Ac-PL3-Asp-Npg-B5-Asp-Asp-Ala-Ala-Phe-Asn-PyrS1-2C1F-BztA-dThr-NH2 | 931 |
| I-1257 | Ac-PL3-Asp-Npg-B5-Asp-Asp-Ala-Ala-Phe-Asn-PyrS2-3Thi-BztA-Gln-NHBn | 932 |
| I-1258 | MeSO2-PL3-Asp-Npg-B5-Asp-Asp-Ala-Ala-Phe-Asn-PyrS2-2C1F-BztA-dThr-NH2 | 933 |
| I-1259 | MeOPr-PL3-Asp-Npg-B5-Asp-Asp-Ala-Ala-Phe-Asn-PyrS2-2C1F-BztA-dThr-NH2 | 934 |
| I-1260 | MeSO2-PL3-Asp-Npg-B5-Asp-Asp-Ala-Ala-Phe-Thr-S8-2C1F-BztA-Gln-NH2 | 935 |
| I-1261 | MeOPr-PL3-Asp-Npg-B5-Asp-Asp-Ala-Ala-Phe-Thr-S8-2C1F-BztA-Gln-NH2 | 936 |
| I-1262 | Ac-PL3-Asp-Npg-B5-Asp-Asp-Ala-Ala-Phe-Thr-S10-2C1F-BztA-Thr-NH2 | 937 |
| I-1263 | Ac-PL3-Asp-Npg-B5-Asp-Thr-Ala-Ala-Phe-Thr-PyrS2-Phe-BztA-Ser-NH2 | 938 |
| I-1264 | Ac-PL3-Asp-Npg-B5-Asp-Thr-Leu-Ala-Phe-Thr-PyrS2-Phe-BztA-Ser-NH2 | 939 |
| I-1265 | Ac-PL3-Asp-Npg-B5-Asp-Thr-Ala-Leu-Phe-Thr-PyrS2-Phe-BztA-Ser-NH2 | 940 |
| I-1266 | Ac-PL3-Asp-Npg-B5-Asp-Thr-Ala-Ala-1NapA-Thr-PyrS2-Phe-BztA-Ser-NH2 | 941 |
| I-1267 | Ac-PL3-Asp-Npg-B5-Asp-Thr-Ala-Ala-2NapA-Thr-PyrS2-Phe-BztA-Ser-NH2 | 942 |
| I-1268 | Ac-PL3-Asp-Npg-B5-Asp-Thr-Ala-Ala-3F3MeF-Thr-PyrS2-Phe-BztA-Ser-NH2 | 943 |
| I-1269 | Ac-PL3-Asp-Npg-B5-Asp-Thr-Ala-Ala-Phe-Thr-PyrS2-1NapA-BztA-Ser-NH2 | 944 |
| I-1270 | Ac-PL3-Asp-Npg-B5-Asp-Thr-Ala-Ala-Phe-Thr-PyrS2-2NapA-BztA-Ser-NH2 | 945 |
| I-1271 | Ac-PL3-Asp-Npg-B5-Asp-Thr-Ala-Ala-Phe-Thr-PyrS2-3F3MeF-BztA-Ser-NH2 | 946 |
| I-1272 | Ac-PL3-Asp-Npg-B5-Asp-Thr-Ala-Ala-Phe-Thr-PyrS2-Phe-1NapA-Ser-NH2 | 947 |
| I-1273 | Ac-PL3-Asp-Npg-B5-Asp-Asp-iPrLys-Ala-Phe-Thr-PyrS2-2C1F-BztA-Thr-NH2 | 948 |
| I-1274 | Ac-PL3-Asp-Npg-B5-Asp-Asp-iPrLys-Ala-Phe-Thr-PyrS2-2C1F-BztA-Leu-NH2 | 949 |
| I-1275 | Ac-PL3-Asp-Npg-B5-Asp-Asp-iPrLys-Ala-Phe-Leu-PyrS2-2C1F-BztA-Gln-NH2 | 950 |
| I-1276 | Ac-PL3-Asp-Npg-B5-Asp-Asp-iPrLys-Ala-Phe-Leu-PyrS2-2C1F-BztA-Thr-NH2 | 951 |
| I-1277 | Ac-PL3-Asp-Npg-B5-Asp-Asp-iPrLys-Ala-Phe-Leu-PyrS2-2C1F-BztA-Leu-NH2 | 952 |
| I-1278 | Ac-PL3-Asp-Npg-B5-Asp-Asp-Ala-Ala-Phe-iPrLys-PyrS2-2C1F-BztA-Gln-NH2 | 953 |
| I-1279 | Ac-PL3-Asp-Npg-B5-Asp-Asp-Ala-Ala-Phe-iPrLys-PyrS2-2C1F-BztA-Thr-NH2 | 954 |
| I-1280 | Ac-PL3-Asp-Npg-B5-Asp-Asp-Ala-Ala-Phe-iPrLys-PyrS2-2C1F-BztA-Leu-NH2 | 955 |
| I-1281 | Ac-PL3-Asp-Npg-B5-Asp-Glu-Ala-Ala-Phe-iPrLys-PyrS2-2C1F-BztA-Gln-NH2 | 956 |
| I-1282 | Ac-PL3-Asp-Npg-B5-Asp-Glu-Ala-Ala-Phe-iPrLys-PyrS2-2C1F-BztA-Thr-NH2 | 957 |
| I-1283 | Ac-PL3-Asp-Npg-B5-Asp-Glu-Ala-Ala-Phe-iPrLys-PyrS2-2C1F-BztA-Leu-NH2 | 958 |
| I-1284 | Ac-PL3-Asn-Npg-B5-Asp-Thr-Ala-Ala-Phe-Leu-PyrS2-Phe-BztA-Gln-NH2 | 959 |
| I-1285 | Ac-PL3-Asn-Npg-B5-Asp-Thr-Ala-Ala-Phe-Leu-PyrS2-Phe-BztA-Gln-NH2 | 959 |
| I-1286 | Ac-PL3-Asn-Ala-B5-Asp-Thr-Ala-Ala-Phe-Leu-PyrS2-Phe-BztA-Gln-NH2 | 960 |
| I-1287 | Ac-PL3-Asn-Ala-B5-Asp-Thr-Ala-Ala-Phe-Leu-PyrS2-Phe-BztA-Gln-NH2 | 960 |
| I-1288 | Ac-PL3-Asn-Npg-B5-Asp-Thr-iPrLys-Ala-Phe-Leu-PyrS2-Phe-BztA-Gln-NH2 | 961 |
| I-1289 | Ac-PL3-Asn-Npg-B5-Asp-Thr-iPrLys-Ala-Phe-Leu-PyrS2-Phe-BztA-Gln-NH2 | 961 |
| I-1290 | Ac-PL3-Asn-Npg-B5-Asp-Thr-Ala-Ala-Phe-Leu-PyrS2-Phe-1NapA-Gln-NH2 | 962 |
| I-1291 | Ac-PL3-Asn-Npg-B5-Asp-Thr-Ala-Ala-Phe-Leu-PyrS2-Phe-2NapA-Gln-NH2 | 963 |
| I-1292 | Ac-PL3-Asn-Npg-B5-Asp-Thr-Ala-Ala-Phe-Leu-PyrS2-Phe-2NapA-Gln-NH2 | 963 |
| I-1293 | Ac-PL3-Asn-Npg-B5-Asp-Thr-Ala-Ala-Phe-Leu-PyrS2-Phe-BztA-Thr-NH2 | 964 |
| I-1294 | Ac-PL3-Asp-Npg-B5-Asn-Thr-Ala-Ala-Phe-Leu-PyrS2-Phe-BztA-Gln-NH2 | 965 |
| I-1295 | Ac-PL3-Asp-Ala-B5-Asn-Thr-Ala-Ala-Phe-Leu-PyrS2-Phe-BztA-Gln-NH2 | 966 |
| I-1296 | Ac-PL3-Asp-Ala-B5-Asn-Thr-Ala-Ala-Phe-Leu-PyrS2-Phe-BztA-Gln-NH2 | 966 |
| I-1297 | Ac-PL3-Asp-Npg-B5-Asn-Thr-iPrLys-Ala-Phe-Leu-PyrS2-Phe-BztA-Gln-NH2 | 967 |
| I-1298 | Ac-PL3-Asp-Npg-B5-Asn-Thr-Ala-Ala-Phe-Leu-PyrS2-Phe-1NapA-Gln-NH2 | 968 |
| I-1299 | Ac-PL3-Asp-Npg-B5-Asn-Thr-Ala-Ala-Phe-Leu-PyrS2-Phe-BztA-Thr-NH2 | 969 |
| I-1300 | Ac-PL3-Asp-Npg-B5-Asn-Thr-Ala-Ala-Phe-Leu-PyrS2-Phe-BztA-Thr-NH2 | 969 |
| I-1301 | Ac-PL3-Asn-Npg-B5-Asn-Thr-Ala-Ala-Phe-Leu-PyrS2-Phe-BztA-Gln-NH2 | 970 |
| I-1302 | Ac-PL3-Asp-Npg-B5-Asp-Asp-Ala-Ala-Phe-Leu-PyrS2-3Thi-BztA-Thro1 | 971 |
| I-1303 | Ac-PL3-Asp-Npg-B5-Asp-Asp-Ala-Ala-Phe-Phe-PyrS2-3Thi-BztA-Thro1 | 972 |
| I-1304 | Ac-PL3-Asn-Npg-B5-Asp-Asp-Ala-Ala-Phe-Leu-PyrS2-3Thi-BztA-Thro1 | 973 |
| I-1305 | Ac-PL3-Asp-Npg-B5-Asp-Asp-Ala-Ala-Phe-Hse-PyrS2-3Thi-BztA-Thro1 | 974 |
| I-1306 | Ac-PL3-Asp-Npg-B5-Asp-Asp-Ala-Ala-Phe-Thr-PyrS2-3Thi-BztA-Thro1 | 975 |
| I-1307 | Ac-PL3-Asp-Cha-B5-Asp-Asp-Ala-Ala-Phe-Leu-PyrS2-2C1F-BztA-Thro1 | 976 |
| I-1308 | Ac-PL3-Asp-Npg-B5-Asp-Asp-Ala-Ala-Phe-Thr-S8-2C1F-BztA-Thro1 | 977 |
| I-1309 | Ac-PL3-Asp-Npg-B5-Asp-Thr-Ala-Ala-Phe-Thr-PyrS2-2C1F-BztA-Thro1 | 978 |
| I-1310 | Ac-PL3-Asp-Cha-B5-Asp-Thr-Ala-Ala-Phe-Thr-PyrS2-2ClF-BztA-Thro1 | 979 |
| I-1311 | Ac-PL3-Asp-Npg-B5-Asp-Asp-Ala-Ala-Phe-Phe-PyrS2-2C1F-BztA-Thro1 | 980 |
| I-1312 | Ac-PL3-Asp-Cha-B5-Asp-Asp-Ala-Ala-Phe-Thr-PyrS2-2C1F-BztA-Thro1 | 981 |
| I-1313 | Ac-PL3-Asp-Npg-B5-Asp-Asp-iPrLys-Ala-Phe-Leu-PyrS2-2C1F-BztA-Thro1 | 982 |
| I-1314 | Ac-PL3-Asp-Npg-B5-Asp-Asp-Ala-Ala-Phe-Asn-PyrS2-2cbmF-BztA-Gln-NH2 | 983 |
| I-1315 | Ac-PL3-Asp-Npg-B5-Asp-Asp-Ala-Ala-Phe-Leu-PyrS2-2cbmF-BztA-Gln-NH2 | 984 |
| I-1316 | Ac-PL3-Asp-Npg-B5-Asp-Asp-Ala-Ala-Phe-Phe-PyrS2-2cbmF-BztA-Gln-NH2 | 985 |
| I-1317 | Ac-PL3-Asp-Npg-B5-Asp-Asp-Ala-Ala-Phe-Asn-PyrS2-2F3MeF-BztA-Gln-NH2 | 986 |
| I-1318 | Ac-PL3-Asp-Npg-B5-Asp-Asp-Ala-Ala-Phe-Leu-PyrS2-2F3MeF-BztA-Gln-NH2 | 987 |
| I-1319 | Ac-PL3-Asp-Npg-B5-Asp-Asp-Ala-Ala-Phe-Phe-PyrS2-2F3MeF-BztA-Gln-NH2 | 988 |
| I-1320 | Ac-PL3-Asp-Npg-B5-Asp-Asp-Ala-Ala-Phe-Asn-PyrS2-2FF-BztA-Gln-NH2 | 989 |
| I-1321 | Ac-PL3-Asp-Npg-B5-Asp-Asp-Ala-Ala-Phe-Leu-PyrS2-2FF-BztA-Gln-NH2 | 990 |
| I-1322 | Ac-PL3-Asp-Npg-B5-Asp-Asp-Ala-Ala-Phe-Phe-PyrS2-2FF-BztA-Gln-NH2 | 991 |
| I-1323 | Ac-PL3-Asp-Npg-B5-Asp-Asp-Ala-Ala-Phe-Asn-PyrS2-2BrF-BztA-Gln-NH2 | 992 |
| I-1324 | PL3-Asp-Npg-B5-Asp-Asp-Ala-Ala-Phe-Leu-PyrS2-2BrF-BztA-Gln-NH2 | 993 |
| I-1325 | PL3-Asp-Npg-B5-Asp-Asp-Ala-Ala-Phe-Phe-PyrS2-2BrF-BztA-Gln-NH2 | 994 |
| I-1326 | PL3-Asp-Npg-B5-Asp-Asp-Ala-Ala-Phe-Phe-PyrS2-2MeF-BztA-Gln-NH2 | 995 |
| I-1327 | PL3-Asp-Npg-B5-Asp-Asp-Ala-Ala-Phe-Leu-PyrS2-2OMeF-BztA-Gln-NH2 | 996 |
| I-1328 | PL3-Asp-Npg-B5-Asp-Asp-Ala-Ala-Phe-Phe-PyrS2-2OMeF-BztA-Gln-NH2 | 997 |
| I-1329 | PL3-Asp-Npg-B5-Asp-Asp-Ala-Ala-Phe-Asn-PyrS2-Cha-BztA-Gln-NH2 | 998 |
| I-1330 | PL3-Asp-Npg-B5-Asp-Asp-Ala-Ala-Phe-Leu-PyrS2-Cha-BztA-Gln-NH2 | 999 |
| I-1331 | PL3-Asp-Npg-B5-Asp-Asp-Ala-Ala-Phe-Phe-PyrS2-Cha-BztA-Gln-NH2 | 1000 |
| I-1332 | PL3-Asp-Npg-B5-Asp-3COOHF-Ala-Ala-Phe-Leu-PyrS2-2C1F-BztA-Gln-NH2 | 1001 |
| I-1333 | PL3-Asp-Npg-B5-Asp-3COOHF-Ala-Ala-Phe-Leu-PyrS2-2C1F-BztA-Ser-NH2 | 1002 |
| I-1334 | PL3-Asp-Npg-B5-Asp-3COOHF-Ala-Ala-Phe-Leu-PyrS2-2C1F-BztA-Thro1 | 1003 |
| I-1335 | PL3-Asp-Npg-B5-Asp-3COOHF-Ala-Ala-Phe-Leu-PyrS2-2F3MeF-BztA-Gln-NH2 | 1004 |
| I-1336 | PL3-Asp-Npg-B5-Asp-3COOHF-Ala-Ala-Phe-Leu-PyrS2-2F3MeF-BztA-Ser-NH2 | 1005 |
| I-1337 | PL3-Asp-Npg-B5-Asp-3COOHF-Ala-Ala-Phe-Leu-PyrS2-2F3MeF-BztA-Thro1 | 1006 |
| I-1338 | PL3-Asp-Npg-B5-Asp-3COOHF-Ala-Ala-Phe-Phe-PyrS2-2C1F-BztA-Gln-NH2 | 1007 |
| I-1339 | PL3-Asp-Npg-B5-Asp-3COOHF-Ala-Ala-Phe-Phe-PyrS2-2C1F-BztA-Ser-NH2 | 1008 |
| I-1340 | PL3-Asp-Npg-B5-Asp-3COOHF-Ala-Ala-Phe-Phe-PyrS2-2C1F-BztA-Thro1 | 1009 |
| I-1341 | PL3-Asp-Npg-B5-Asp-3COOHF-Ala-Ala-Phe-Phe-PyrS2-2F3MeF-BztA-Gln-NH2 | 1010 |
| I-1342 | PL3-Asp-Npg-B5-Asp-3COOHF-Ala-Ala-Phe-Phe-PyrS2-2F3MeF-BztA-Ser-NH2 | 1011 |
| I-1343 | Ac-PL3-Asp-Npg-B5-Asp-3COOHF-Ala-Ala-Phe-Phe-PyrS2-2F3MeF-BztA-Thro1 | 1012 |
| I-1344 | PL3-Asp-Npg-B5-Asp-Asp-Ala-Ala-Phe-Leu-PyrS2-2Thi-BztA-Gln-NH2 | 1013 |
| I-1345 | PL3-Asp-Npg-B5-Asp-Asp-Ala-Ala-Phe-Phe-PyrS2-2Thi-BztA-Gln-NH2 | 1014 |
| I-1346 | PL3-Asp-Npg-B5-Asp-Asp-Ala-Ala-Phe-Asn-PyrS2-dPhe-BztA-Gln-NH2 | 1015 |
| I-1347 | PL3-Asp-Npg-B5-Asp-Asp-Ala-Ala-Phe-Leu-PyrS2-dPhe-BztA-Gln-NH2 | 1016 |
| I-1348 | PL3-Asp-Npg-B5-Asp-Asp-Ala-Ala-Phe-Phe-PyrS2-dPhe-BztA-Gln-NH2 | 1017 |
| I-1349 | PL3-Asp-Npg-B5-Asp-Asp-Ala-Ala-Phe-Asn-PyrS2-hPhe-BztA-Gln-NH2 | 1018 |
| I-1350 | PL3-Asp-Npg-B5-Asp-Asp-Ala-Ala-Phe-Leu-PyrS2-hPhe-BztA-Gln-NH2 | 1019 |
| I-1351 | PL3-Asp-Npg-B5-Asp-Asp-Ala-Ala-Phe-Phe-PyrS2-hPhe-BztA-Gln-NH2 | 1020 |
| I-1352 | PL3-Asp-Npg-B5-Asp-Asp-Ala-Ala-Phe-Asn-PyrS2-CypA-BztA-Gln-NH2 | 1021 |
| I-1353 | PL3-Asp-Npg-B5-Asp-Asp-Ala-Ala-Phe-Leu-PyrS2-CypA-BztA-Gln-NH2 | 1022 |
| I-1354 | NdiMeButC-PL3-Asp-Npg-B5-Asp-Asp-Ala-Ala-Phe-Ser-PyrS2-Phe-BztA-Ser-NH2 | 1023 |
| I-1355 | Oct-PL3-Asp-Npg-B5-Asp-Asp-Ala-Ala-Phe-Ser-PyrS2-Phe-BztA-Ser-NH2 | 1024 |
| I-1356 | PL3-Asp-Npg-B5-Asp-Asp-Ala-Ala-Phe-Ser-PyrS2-Phe-BztA-Ser-NH2 | 1025 |
| I-1357 | PL3-Asp-Npg-B5-Asp-Asp-Ala-Ala-Phe-Ser-PyrS2-Phe-BztA-Ser-NH2 | 1025 |
| I-1358 | NdiMeButC-PL3-Asp-Npg-B5-Asp-Asp-Ala-Ala-Phe-Leu-PyrS2-Phe-BztA-Gln-NH2 | 1026 |
| I-1359 | NdiMeButC-PL3-Asp-Npg-B5-Asp-Asp-Ala-Ala-Phe-Leu-PyrS2-Phe-BztA-Gln-NH2 | 1026 |
| I-1360 | PL3-Asp-Npg-B5-Asp-Asp-Ala-Ala-Phe-Leu-PyrS2-Phe-BztA-Gln-NH2 | 1027 |
| I-1361 | PL3-Asp-Npg-B5-Asp-Asp-Ala-Ala-Phe-Leu-PyrS2-Phe-BztA-Gln-NH2 | 1027 |
| I-1362 | PL3-Asp-Npg-B5-Asp-Asp-Ala-Ala-Phe-Leu-PyrS2-Phe-BztA-Gln-NH2 | 1027 |
| I-1363 | Ac-PL3-Asp-Npg-B5-Asp-Asp-Ala-Ala-Phe-2F3MeF-PyrS2-3Thi-BztA-Gln-NH2 | 1028 |
| I-1364 | Ac-PL3-Asp-Npg-B5-Asp-Asp-Ala-Ala-Phe-3F3MeF-PyrS2-3Thi-BztA-Gln-NH2 | 1029 |
| I-1365 | Ac-PL3-Asp-Npg-B5-Asp-Asp-Ala-Ala-Phe-4F3MeF-PyrS2-3Thi-BztA-Gln-NH2 | 1030 |
| I-1366 | Ac-PL3-Asp-Npg-B5-Asp-Asp-Ala-Ala-Phe-Phe-PyrS2-2C1F-BztA-Gln-NH2 | 1031 |
| I-1367 | Ac-PL3-Asp-Npg-B5-Asp-Asp-Ala-Ala-Phe-2F3MeF-PyrS2-2C1F-BztA-Gln-NH2 | 1032 |
| I-1368 | Ac-PL3-Asp-Npg-B5-Asp-Asp-Ala-Ala-Phe-3F3MeF-PyrS2-2C1F-BztA-Gln-NH2 | 1033 |
| I-1369 | Ac-PL3-Asp-Npg-B5-Asp-Asp-Ala-Ala-Phe-4F3MeF-PyrS2-2C1F-BztA-Gln-NH2 | 1034 |
| I-1370 | Ac-PL3-Asp-Npg-B5-Asp-Asp-Ala-Ala-Phe-2F3MeF-PyrS2-3Thi-BztA-Thro1 | 1035 |
| I-1371 | Ac-PL3-Asp-Npg-B5-Asp-Asp-Ala-Ala-Phe-3F3MeF-PyrS2-3Thi-BztA-Thro1 | 1036 |
| I-1372 | Ac-PL3-Asp-Npg-B5-Asp-Asp-Ala-Ala-Phe-4F3MeF-PyrS2-3Thi-BztA-Thro1 | 1037 |
| I-1373 | PL3-Asp-Npg-B5-Asp-Asp-Ala-Ala-Phe-Asn-PyrS2-3Thi-BztA-Gln-NEt2 | 1038 |
| I-1374 | PL3-Asp-Npg-B5-Asp-Asp-Ala-Ala-Phe-Asn-PyrS2-3Thi-BztA-Gln-NHCyHe | 1039 |
| I-1375 | PL3-Asp-Npg-B5-Asp-Asp-Ala-Ala-Phe-Asn-PyrS2-3Thi-BztA-Gln-NHCyPr | 1040 |
| I-1376 | Ac-PL3-Asp-Ala-B5-Asn-Thr-iPrLys-Ala-Phe-Leu-PyrS2-Phe-BztA-Gln-NH2 | 1041 |
| I-1377 | Ac-PL3-Asp-Ala-B5-Asn-Thr-iPrLys-Ala-Phe-Leu-PyrS2-Phe-BztA-Lys-NH2 | 1042 |
| I-1378 | Ac-PL3-Asp-Ala-B5-Asn-Thr-iPrLys-Ala-Phe-Leu-PyrS2-Phe-BztA-Thr-NH2 | 1043 |
| I-1379 | Ac-PL3-Asp-Ala-B5-Asn-Gln-iPrLys-Ala-Phe-Leu-PyrS2-Phe-BztA-Gln-NH2 | 1044 |
| I-1380 | Ac-PL3-Asp-Ala-B5-Asn-Lys-iPrLys-Ala-Phe-Leu-PyrS2-Phe-BztA-Gln-NH2 | 1045 |
| I-1381 | Ac-PL3-Asp-Ala-B5-Asn-Thr-iPrLys-Ala-Phe-Leu-PyrS2-Phe-2NapA-Gln-NH2 | 1046 |
| I-1382 | Ac-PL3-Asn-Ala-B5-Asp-Thr-iPrLys-Ala-Phe-Leu-PyrS2-Phe-BztA-Gln-NH2 | 1047 |
| I-1383 | Ac-PL3-Asn-Ala-B5-Asp-Thr-iPrLys-Ala-Phe-Leu-PyrS2-Phe-BztA-Lys-NH2 | 1048 |
| I-1384 | Ac-PL3-Asn-Ala-B5-Asp-Thr-iPrLys-Ala-Phe-Leu-PyrS2-Phe-BztA-Thr-NH2 | 1049 |
| I-1385 | Ac-PL3-Asn-Ala-B5-Asp-Gln-iPrLys-Ala-Phe-Leu-PyrS2-Phe-BztA-Gln-NH2 | 1050 |
| I-1386 | Ac-PL3-Asn-Ala-B5-Asp-Lys-iPrLys-Ala-Phe-Leu-PyrS2-Phe-BztA-Gln-NH2 | 1051 |
| I-1387 | Ac-PL3-Asn-Ala-B5-Asp-Thr-iPrLys-Ala-Phe-Leu-PyrS2-Phe-2NapA-Gln-NH2 | 1052 |
| I-1388 | Ac-PL3-Asp-Npg-B5-Asp-Asp-Ala-Ala-Phe-Hse-S8-Phe-BztA-Gln-NH2 | 1053 |
| I-1389 | Ac-PL3-Asp-Npg-B5-Asp-Asp-Ala-Ala-Phe-Hse-S8-3Thi-BztA-Gln-NH2 | 1054 |
| I-1390 | Ac-PL3-Asp-Npg-B5-Asp-Asp-Ala-Ala-Phe-Hse-S8-3Thi-BztA-Thro1 | 1055 |
| I-1391 | Ac-PL3-Asp-Npg-B5-Asp-Asp-Ala-Ala-Phe-Asn-S8-2Thi-BztA-Gln-NH2 | 1056 |
| I-1392 | Ac-PL3-Asp-Npg-B5-Asp-Asp-Ala-Ala-Phe-Hse-S8-2Thi-BztA-Gln-NH2 | 1057 |
| I-1393 | Ac-PL3-Asp-Npg-B5-Asp-Asp-Ala-Ala-Phe-Hse-S8-2Thi-BztA-Thro1 | 1058 |
| I-1394 | Ac-PL3-Asp-Npg-B5-Asp-Asp-Ala-Ala-Phe-Asn-S8-2C1F-BztA-Gln-NH2 | 1059 |
| I-1395 | Ac-PL3-Asp-Npg-B5-Asp-Asp-Ala-Ala-Phe-Hse-S8-2C1F-BztA-Gln-NH2 | 1060 |
| I-1396 | Ac-PL3-Asp-Npg-B5-Asp-Asp-Ala-Ala-Phe-Asn-S8-2F3MeF-BztA-Gln-NH2 | 1061 |
| I-1397 | Ac-PL3-Asp-Npg-B5-Asp-Asp-Ala-Ala-Phe-Hse-S8-2F3MeF-BztA-Gln-NH2 | 1062 |
| I-1398 | Ac-PL3-Asp-Npg-B5-Asp-Asp-Ala-Ala-Phe-Hse-S8-2F3MeF-BztA-Thro1 | 1063 |
| I-1399 | Ac-PL3-Asp-Leu-B5-Asp-Asp-Ala-Ala-Phe-Leu-PyrS2-3Thi-BztA-Gln-NH2 | 1064 |
| I-1400 | Ac-PL3-Asp-nLeu-B5-Asp-Asp-Ala-Ala-Phe-Leu-PyrS2-3Thi-BztA-Gln-NH2 | 1065 |
| I-1401 | Ac-PL3-Asp-nLeu-B5-Asp-Asp-Ala-Ala-Phe-nLeu-PyrS2-3Thi-BztA-Gln-NH2 | 1066 |
| I-1402 | Ac-PL3-Asp-Leu-B5-Asp-Asp-Ala-Ala-Phe-Leu-PyrS2-Phe-BztA-Gln-NH2 | 1067 |
| I-1403 | Ac-PL3-Asp-nLeu-B5-Asp-Asp-Ala-Ala-Phe-Leu-PyrS2-Phe-BztA-Gln-NH2 | 1068 |
| I-1404 | Ac-PL3-Asp-Leu-B5-Asp-Asp-Ala-Ala-Phe-nLeu-PyrS2-Phe-BztA-Gln-NH2 | 1069 |
| I-1405 | Ac-PL3-Asp-nLeu-B5-Asp-Asp-Ala-Ala-Phe-nLeu-PyrS2-Phe-BztA-Gln-NH2 | 1070 |
| I-1406 | Ac-PL3-Asp-Leu-B5-Asp-3COOHF-Ala-Ala-Phe-Leu-PyrS2-3Thi-BztA-Gln-NH2 | 1071 |
| I-1407 | Ac-PL3-Asp-Leu-B5-Asp-3COOHF-Ala-Ala-Phe-nLeu-PyrS2-3Thi-BztA-Gln-NH2 | 1072 |
| I-1408 | Ac-PL3-Asp-nLeu-B5-Asp-3COOHF-Ala-Ala-Phe-nLeu-PyrS2-3Thi-BztA-Gln-NH2 | 1073 |
| I-1409 | Ac-PL3-Asp-Npg-B5-Asp-Asp-Ala-Ala-Phe-Leu-PyrS2-3Thi-BztA-Leu-NH2 | 1074 |
| I-1410 | Ac-PL3-Asp-Leu-B5-Asp-Asp-Ala-Ala-Phe-Leu-PyrS2-3Thi-BztA-Leu-NH2 | 1075 |
| I-1411 | Ac-PL3-Asp-nLeu-B5-Asp-Asp-Ala-Ala-Phe-Leu-PyrS2-3Thi-BztA-Leu-NH2 | 1076 |
| I-1412 | Ac-PL3-Asp-nLeu-B5-Asp-Asp-Ala-Ala-Phe-Leu-PyrS2-3Thi-BztA-Leu-NH2 | 1076 |
| I-1413 | Ac-PL3-Asp-Asn-B5-Asp-Asp-Ala-Ala-Phe-Leu-PyrS2-3Thi-BztA-Leu-NH2 | 1077 |
| I-1414 | Ac-PL3-Asp-Ser-B5-Asp-Asp-Ala-Ala-Phe-Leu-PyrS2-3Thi-BztA-Leu-NH2 | 1078 |
| I-1415 | Ac-PL3-Asp-Thr-B5-Asp-Asp-Ala-Ala-Phe-Leu-PyrS2-3Thi-BztA-Leu-NH2 | 1079 |
| I-1416 | Ac-PL3-Asp-Npg-B5-Asp-Asp-Ala-Ala-Phe-Leu-PyrS2-2C1F-BztA-Leu-NH2 | 1080 |
| I-1417 | Ac-PL3-Asp-Npg-B5-Asp-Asp-Ala-Ala-Phe-Leu-PyrS2-2C1F-BztA-Leu-NH2 | 1080 |
| I-1418 | Ac-PL3-Asp-Leu-B5-Asp-Asp-Ala-Ala-Phe-Leu-PyrS2-2C1F-BztA-Leu-NH2 | 1081 |
| I-1419 | Ac-PL3-Asp-Leu-B5-Asp-Asp-Ala-Ala-Phe-Leu-PyrS2-2C1F-BztA-Leu-NH2 | 1081 |
| I-1420 | Ac-PL3-Asp-nLeu-B5-Asp-Asp-Ala-Ala-Phe-Leu-PyrS2-2C1F-BztA-Leu-NH2 | 1082 |
| I-1421 | Ac-PL3-Asp-nLeu-B5-Asp-Asp-Ala-Ala-Phe-Leu-PyrS2-2C1F-BztA-Leu-NH2 | 1082 |
| I-1422 | Ac-PL3-Asp-Asn-B5-Asp-Asp-Ala-Ala-Phe-Leu-PyrS2-2C1F-BztA-Leu-NH2 | 1083 |
| I-1423 | Ac-PL3-Asp-Ser-B5-Asp-Asp-Ala-Ala-Phe-Leu-PyrS2-2C1F-BztA-Leu-NH2 | 1084 |
| I-1424 | Ac-PL3-Asp-Thr-B5-Asp-Asp-Ala-Ala-Phe-Leu-PyrS2-2C1F-BztA-Leu-NH2 | 1085 |
| I-1425 | Ac-PL3-Asp-Npg-B5-Asp-Asp-Ala-Ala-Phe-Leu-PyrS2-3Thi-23FF-Gln-NH2 | 1086 |
| I-1426 | Ac-PL3-Asp-Npg-B5-Asp-Asp-Ala-Ala-Phe-Leu-PyrS2-3Thi-23FF-Ser-NH2 | 1087 |
| I-1427 | Ac-PL3-Asp-Npg-B5-Asp-Asp-Ala-Ala-Phe-Leu-PyrS2-3Thi-34FF-Gln-NH2 | 1088 |
| I-1428 | Ac-PL3-Asp-Npg-B5-Asp-Asp-Ala-Ala-Phe-Leu-PyrS2-3Thi-34FF-Ser-NH2 | 1089 |
| I-1429 | Ac-PL3-Asp-Npg-B5-Asp-Asp-Ala-Ala-Phe-Leu-PyrS2-3Thi-34OMeF-Gln-NH2 | 1090 |
| I-1430 | Ac-PL3-Asp-Npg-B5-Asp-Asp-Ala-Ala-Phe-Leu-PyrS2-3Thi-34OMeF-Ser-NH2 | 1091 |
| I-1431 | Ac-PL3-Asp-Npg-B5-Asp-Asp-Ala-Ala-Phe-Leu-PyrS2-3Thi-Trp-Gln-NH2 | 1092 |
| I-1432 | Ac-PL3-Asp-Npg-B5-Asp-Asp-Ala-Ala-Phe-Leu-PyrS2-3Thi-Trp-Ser-NH2 | 1093 |
| I-1433 | Ac-PL3-Asp-Npg-B5-Asp-Asp-Ala-Ala-Phe-Leu-PyrS2-3Thi-1MeW-Gln-NH2 | 1094 |
| I-1434 | Ac-PL3-Asp-Npg-B5-Asp-Asp-Ala-Ala-Phe-Leu-PyrS2-3Thi-1MeW-Ser-NH2 | 1095 |
| I-1435 | Ac-PL3-Asp-Npg-B5-Asp-Asp-Ala-Ala-Phe-Leu-PyrS2-3Thi-6C1W-Gln-NH2 | 1096 |
| I-1436 | Ac-PL3-Asp-Npg-B5-Asp-Asp-Ala-Ala-Phe-Leu-PyrS2-3Thi-6C1W-Ser-NH2 | 1097 |
| I-1437 | Ac-PL3-Asp-Npg-B5-Asp-Glu-Ala-Ala-Phe-Leu-PyrS2-3Thi-BztA-Gln-NH2 | 1098 |
| I-1438 | Ac-PL3-Asp-Npg-B5-Asp-Aad-Ala-Ala-Phe-Leu-PyrS2-3Thi-BztA-Gln-NH2 | 1099 |
| I-1439 | Ac-PL3-Asp-Npg-B5-Asp-Trp-Ala-Ala-Phe-Leu-PyrS2-3Thi-BztA-Gln-NH2 | 1100 |
| I-1440 | Ac-PL3-Asp-Npg-B5-Asp-Gln-Ala-Ala-Phe-Leu-PyrS2-3Thi-BztA-Gln-NH2 | 1101 |
| I-1441 | Ac-PL3-Asp-Npg-B5-Asp-Phe-Ala-Ala-Phe-Leu-PyrS2-3Thi-BztA-Gln-NH2 | 1102 |
| I-1442 | Ac-PL3-Asp-Npg-B5-Asp-Val-Ala-Ala-Phe-Leu-PyrS2-3Thi-BztA-Gln-NH2 | 1103 |
| I-1443 | Ac-PL3-Asp-Npg-B5-Asp-Ser-Ala-Ala-Phe-Leu-PyrS2-3Thi-BztA-Gln-NH2 | 1104 |
| I-1444 | Ac-PL3-Asp-Npg-B5-Asp-Leu-Ala-Ala-Phe-Leu-PyrS2-3Thi-BztA-Gln-NH2 | 1105 |
| I-1445 | Ac-PL3-Asp-Npg-B5-Asp-1AcAW-Ala-Ala-Phe-Leu-PyrS2-3Thi-BztA-Gln-NH2 | 1106 |
| I-1446 | Ac-PL3-Asp-Npg-B5-Asp-[Tfb]GA-Ala-Ala-Phe-Asn-PyrS2-2C1F-BztA-Gln-NH2 | 1107 |
| I-1447 | Ac-PL3-Asp-Npg-B5-Asp-[Bn]GA-Ala-Ala-Phe-Asn-PyrS2-2C1F-BztA-Gln-NH2 | 1108 |
| I-1448 | Ac-PL3-Asp-Npg-B5-Asp-[Pfbn]GA-Ala-Ala-Phe-Asn-PyrS2-2C1F-BztA-Gln-NH2 | 1109 |
| I-1449 | Ac-PL3-Asp-Npg-B5-Asp-[Pfbn]GA-Ala-Ala-Phe-Asn-PyrS2-2C1F-BztA-Gln-NH2 | 1109 |
| I-1450 | Ac-PL3-Asp-Npg-B5-Asp-[Pfbn]GA-Ala-Ala-Phe-Asn-PyrS2-2C1F-BztA-Gln-NH2 | 1109 |
| I-1451 | Ac-PL3-Asp-Npg-B5-Asp-[iPr]GA-Ala-Ala-Phe-Asn-PyrS2-2C1F-BztA-Gln-NH2 | 1110 |
| I-1452 | Ac-PL3-Asp-Npg-B5-Asp-[iPr]GA-Ala-Ala-Phe-Asn-S8-3Thi-BztA-Gln-NH2 | 1111 |
| I-1453 | Ac-PL3-Asp-Npg-B5-Asp-[iPr]GA-Ala-Ala-Phe-Asn-S8-3Thi-BztA-Gln-NH2 | 1111 |
| I-1454 | Ac-PL3-Asp-Npg-B5-Asp-[Tfb]GA-Ala-Ala-Phe-Asn-S8-3Thi-BztA-Gln-NH2 | 1112 |
| I-1455 | Ac-PL3-Asp-Npg-B5-Asp-[Tfb]GA-Ala-Ala-Phe-Asn-S8-3Thi-BztA-Gln-NH2 | 1112 |
| I-1456 | Ac-PL3-Asp-Npg-B5-Asp-[Bn]GA-Ala-Ala-Phe-Asn-S8-3Thi-BztA-Gln-NH2 | 1113 |
| I-1457 | Ac-PL3-Asp-Npg-B5-Asp-[Tfp]Dap-Ala-Ala-Phe-Asn-PyrS2-2C1F-BztA-Gln-NH2 | 1114 |
| I-1458 | Ac-PL3-Asp-Leu-B5-Asp-Glu-Ala-Ala-Phe-Gln-S8-Phe-BztA-Gln-NH2 | 333 |
| I-1459 | Ac-PL3-Asp-Npg-B5-Asp-Asp-Ala-Ala-Phe-Gln-PyrS2-3Thi-BztA-His-NH2 | 501 |
| I-1460 | Ac-PL3-Asp-Npg-B5-Asp-Asp-Ala-Ala-Phe-Gln-PyrS2-3Thi-BztA-His-NH2 | 501 |
| I-1461 | Ac-PL3-Asp-Npg-B5-Asp-TfeGA-Ala-Ala-Phe-Leu-PyrS2-3Thi-BztA-Gln-NH2 | 571 |
| I-1462 | Ac-PL3-Asp-Npg-B5-Asp-3COOHF-Ala-Ala-Phe-Leu-PyrS2-3Thi-BztA-Gln-NH2 | 1115 |
| I-1463 | Ac-PL3-Asp-Npg-B5-Asp-TfeGA-Ala-Ala-Phe-Thr-PyrS2-2C1F-BztA-Gln-NH2 | 639 |
| I-1464 | Ac-PL3-Asp-Npg-B5-Asp-Asp-Ala-Ala-Phe-Asn-PyrS2-2C1F-BztA-Gln-NH2 | 510 |
| I-1465 | Ac-PL3-Asp-Npg-B5-Asn-Thr-iPrLys-Ala-Phe-Leu-PyrS2-Phe-BztA-Gln-NH2 | 967 |
| I-1466 | Ac-PL3-Asp-Npg-B5-Asp-3COOHF-Ala-Ala-Phe-Leu-PyrS2-2F3MeF-BztA-Gln-NH2 | 1116 |
| I-1467 | Ac-PL3-Asp-Npg-B5-Asp-Asp-[CH2NMe2]4SEF-Ala-Phe-Leu-PyrS2-Phe-BztA-Gln | 1117 |
| I-1468 | Ac-PL3-Asp-Npg-B5-Asp-Asp-Ala-Ala-[CH2NMe2]4SEF-Leu-PyrS2-Phe-BztA-Gln | 1118 |
| I-1469 | Ac-PL3-Asp-[lithocholate]-Lys-B5-Asp-Asp-Ala-Ala-Phe-Leu-PyrS2-3Thi-BztA-Gln- | 1119 |
| | NH2 | |
| I-1470 | Ac-PL3-Asp-[lithocholate-PEG2]-Lys-B5-Asp-Asp-Ala-Ala-Phe-Leu-PyrS2-3Thi- | 1120 |
| | BztA-Gln-NH2 | |
| I-1471 | Ac-PL3-Asp-[Me3AdamantC]-Lys-B5-Asp-Asp-Ala-Ala-Phe-Leu-PyrS2-3Thi-BztA- | 1121 |
| | Gln-NH2 | |
| I-1472 | Ac-PL3-Asp-[Me3AdamantC-PEG2]-Lys-B5-Asp-Asp-Ala-Ala-Phe-Leu-PyrS2-3Thi- | 1122 |
| | BztA-Gln-NH2 | |
| I-1473 | Ac-PL3-Asp-Npg-B5-Asp-Asp-Ala-[lithocholate]-Lys-Phe-Leu-PyrS2-3Thi-BztA-Gln- | 1123 |
| | NH2 | |
| I-1474 | Ac-PL3-Asp-Npg-B5-Asp-Asp-Ala-[lithocholate-PEG2]-Lys-Phe-Leu-PyrS2-3Thi- | 1124 |
| | BztA-Gln-NH2 | |
| I-1475 | Ac-PL3-Asp-Npg-B5-Asp-Asp-Ala-[Me3AdamantC]-Lys-Phe-Leu-PyrS2-3Thi-BztA- | 1125 |
| | Gln-NH2 | |
| I-1476 | Ac-PL3-Asp-Npg-B5-Asp-Asp-Ala-[Me3AdamantC-PEG2]-Lys-Phe-Leu-PyrS2-3Thi- | 1126 |
| | BztA-Gln-NH2 | |
| I-1477 | Ac-PL3-Asp-Npg-B5-Asp-Asp-Ala-Ala-Phe-Leu-PyrS2-3Thi-BztA-Gln-[lithocholate]- | 1127 |
| | Lys-NH2 | |
| I-1478 | Ac-PL3-Asp-Npg-B5-Asp-Asp-Ala-Ala-Phe-Leu-PyrS2-3Thi-BztA-Gln-[lithocholate- | 1128 |
| | PEG2]-Lys-NH2 | |
| I-1479 | Ac-PL3-Asp-Npg-B5-Asp-Asp-Ala-Ala-Phe-Leu-PyrS2-3Thi-BztA-Gln- | 1129 |
| | [Me3AdamantC]-Lys-NH2 | |
| I-1480 | Ac-PL3-Asp-Npg-B5-Asp-Asp-Ala-Ala-Phe-Leu-PyrS2-3Thi-BztA-Gln- | 1130 |
| | Me3AdamantC-PEG2]-Lys-NH2 | |
| I-1481 | Ac-PL3-Asp-Npg-B5-Asp-Asp-MorphNva-Ala-Phe-Leu-PyrS2-3Thi-BztA-Gln-NH2 | 1131 |
| I-1482 | Ac-PL3-Asp-Npg-B5-Asp-Asp-F2PipNva-Ala-Phe-Leu-PyrS2-3Thi-BztA-Gln-NH2 | 1132 |
| I-1483 | Ac-PL3-Asp-Npg-B5-Asp-Asp-MorphAsn-Ala-Phe-Leu-PyrS2-3Thi-BztA-Gln-NH2 | 1133 |
| I-1484 | Ac-PL3-Asp-Npg-B5-Asp-Asp-MePpzAsn-Ala-Phe-Leu-PyrS2-3Thi-BztA-Gln-NH2 | 1134 |
| I-1485 | Ac-PL3-Asp-Npg-B5-Asp-Asp-Ala-Ala-Phe-MorphNva-PyrS2-3Thi-BztA-Gln-NH2 | 1135 |
| I-1486 | Ac-PL3-Asp-Npg-B5-Asp-Asp-Ala-Ala-Phe-F2PipNva-PyrS2-3Thi-BztA-Gln-NH2 | 1136 |
| I-1487 | Ac-PL3-Asp-Npg-B5-Asp-Asp-Ala-Ala-Phe-MorphAsn-PyrS2-3Thi-BztA-Gln-NH2 | 1137 |
| I-1488 | Ac-PL3-Asp-Npg-B5-Asp-Asp-Ala-Ala-Phe-MePpzAsn-PyrS2-3Thi-BztA-Gln-NH2 | 1138 |
| I-1489 | Ac-PL3-Asp-Npg-B5-Asp-Asp-[3Py]4SF-Ala-Phe-Leu-PyrS2-Phe-BztA-Gln-NH2 | 1139 |
| I-1490 | Ac-PL3-Asp-Npg-B5-Asp-Asp-Ala-Ala-[3Py]4SF-Leu-PyrS2-Phe-BztA-Gln-NH2 | 1140 |
| I-1491 | Ac-PL3-Asp-Npg-B5-Asp-Asp-Ala-Ala-Phe-Hse-S8-2C1F-BztA-Thro1 | 1141 |
| I-1492 | Ac-PL3-Asp-Npg-B5-Asp-Asp-Ala-Ala-Phe-Leu-PyrS2-3Thi-BztA-Asn-NH2 | 1142 |
| I-1493 | Ac-PL3-Asp-Npg-B5-Asp-Asp-Ala-Ala-Phe-Leu-PyrS2-3Thi-BztA-Ser-NH2 | 1143 |
| I-1494 | Ac-PL3-Asp-Npg-B5-Asp-Asp-Ala-Ala-Phe-Leu-PyrS2-3Thi-BztA-Thr-NH2 | 1144 |
| I-1495 | Ac-PL3-Asp-Npg-B5-Asp-Asp-Ala-Ala-Phe-Leu-PyrS2-3Thi-BztA-Val-NH2 | 1145 |
| I-1496 | Ac-PL3-Asp-Npg-B5-Asp-Asp-Ala-Ala-Phe-Leu-PyrS2-3Thi-BztA-Phe-NH2 | 1146 |
| I-1497 | Ac-PL3-Asp-Npg-B5-Asp-Asp-Ala-Ala-Phe-Leu-PyrS2-3Thi-BztA-Trp-NH2 | 1147 |
| I-1498 | Ac-PL3-Asp-Npg-B5-Asp-Asp-Ala-Ala-Phe-Leu-PyrS2-3Thi-BztA-4TriA-NH2 | 1148 |
| I-1499 | Ac-PL3-Asp-Npg-B5-Asp-Asp-Ala-Ala-Phe-Leu-PyrS2-3Thi-BztA-Hse-NH2 | 1149 |
| I-1500 | Ac-PL3-Asp-Npg-B5-Asp-Asp-Ala-Ala-Phe-Leu-PyrS2-3Thi-BztA-dGln-NH2 | 1150 |
| I-1501 | Ac-PL3-Asp-Npg-B5-Asp-Asp-Ala-MorphNva-Phe-Leu-PyrS2-3Thi-BztA-Gln-NH2 | 1151 |
| I-1502 | Ac-PL3-Asp-Npg-B5-Asp-Asp-Ala-F2PipNva-Phe-Leu-PyrS2-3Thi-BztA-Gln-NH2 | 1152 |
| I-1503 | Ac-PL3-Asp-Npg-B5-Asp-Asp-Ala-MePpzNva-Phe-Leu-PyrS2-3Thi-BztA-Gln-NH2 | 1153 |
| I-1504 | Ac-PL3-Asp-Npg-B5-Asp-Asp-Ala-MorphAbu-Phe-Leu-PyrS2-3Thi-BztA-Gln-NH2 | 1154 |
| I-1505 | Ac-PL3-Asp-Npg-B5-Asp-Asp-Ala-F2PipAbu-Phe-Leu-PyrS2-3Thi-BztA-Gln-NH2 | 1155 |
| I-1506 | Ac-PL3-Asp-Npg-B5-Asp-Asp-Ala-MePpzAbu-Phe-Leu-PyrS2-3Thi-BztA-Gln-NH2 | 1156 |
| I-1507 | Ac-PL3-Asp-Npg-B5-Asp-Asp-Ala-MorphAsn-Phe-Leu-PyrS2-3Thi-BztA-Gln-NH2 | 1157 |
| I-1508 | Ac-PL3-Asp-Npg-B5-Asp-Asp-Ala-MePpzAsn-Phe-Leu-PyrS2-3Thi-BztA-Gln-NH2 | 1158 |
| I-1509 | Ac-PL3-Asp-Phe-B5-Asp-Asp-Ala-Ala-Phe-Leu-PyrS2-3Thi-BztA-Gln-NH2 | 1159 |
| I-1510 | Ac-PL3-Asp-Phe-B5-Asp-Asp-Ala-Ala-Phe-Leu-PyrS2-3Thi-BztA-Hse-NH2 | 1160 |
| I-1511 | Ac-PL3-Asp-Tyr-B5-Asp-Asp-Ala-Ala-Phe-Leu-PyrS2-3Thi-BztA-Gln-NH2 | 1161 |
| I-1512 | Ac-PL3-Asp-Tyr-B5-Asp-Asp-Ala-Ala-Phe-Leu-PyrS2-3Thi-BztA-Hse-NH2 | 1162 |
| I-1513 | Ac-PL3-Asp-Ser-B5-Asp-Asp-Ala-Ala-Phe-Leu-PyrS2-3Thi-BztA-Gln-NH2 | 1163 |
| I-1514 | Ac-PL3-Asp-Ser-B5-Asp-Asp-Ala-Ala-Phe-Leu-PyrS2-3Thi-BztA-Hse-NH2 | 1164 |
| I-1515 | Ac-PL3-Asp-Thr-B5-Asp-Asp-Ala-Ala-Phe-Leu-PyrS2-3Thi-BztA-Gln-NH2 | 1165 |
| I-1516 | Ac-PL3-Asp-Thr-B5-Asp-Asp-Ala-Ala-Phe-Leu-PyrS2-3Thi-BztA-Hse-NH2 | 1166 |
| I-1517 | Ac-PL3-Asp-Asn-B5-Asp-Asp-Ala-Ala-Phe-Leu-PyrS2-3Thi-BztA-Gln-NH2 | 1167 |
| I-1518 | Ac-PL3-Asp-Asn-B5-Asp-Asp-Ala-Ala-Phe-Leu-PyrS2-3Thi-BztA-Hse-NH2 | 1168 |
| I-1519 | Ac-PL3-Asp-Cha-B5-Asp-Asp-Ala-Ala-Phe-Leu-PyrS2-3Thi-BztA-Gln-NH2 | 1169 |
| I-1520 | Ac-PL3-Asp-Cha-B5-Asp-Asp-Ala-Ala-Phe-Leu-PyrS2-3Thi-BztA-Hse-NH2 | 1170 |
| I-1521 | Ac-PL3-Asp-nLeu-B5-Asp-Glu-Ala-Ala-Phe-Gln-S8-Phe-BztA-Gln-NH2 | 1171 |
| I-1522 | Ac-PL3-Asp-nLeu-B5-Asp-Glu-Ala-Ala-Phe-Gln-S8-Phe-BztA-Gln-NH2 | 1171 |
| I-1523 | Ac-PL3-Asp-nLeu-B5-Asp-Glu-Ala-Ala-Phe-Leu-S8-Phe-BztA-Gln-NH2 | 1172 |
| I-1524 | Ac-PL3-Asp-nLeu-B5-Asp-Glu-Ala-Ala-Phe-Hse-S8-Phe-BztA-Gln-NH2 | 1173 |
| I-1525 | Ac-PL3-Asp-nLeu-B5-Asp-Glu-Ala-Ala-Phe-Hse-S8-Phe-BztA-Gln-NH2 | 1173 |
| I-1526 | Ac-PL3-Asp-Ser-B5-Asp-Glu-Ala-Ala-Phe-Gln-S8-Phe-BztA-Gln-NH2 | 1174 |
| I-1527 | Ac-PL3-Asp-Ser-B5-Asp-Glu-Ala-Ala-Phe-Gln-S8-Phe-BztA-Gln-NH2 | 1174 |
| I-1528 | Ac-PL3-Asp-Ser-B5-Asp-Glu-Ala-Ala-Phe-Leu-S8-Phe-BztA-Gln-NH2 | 1175 |
| I-1529 | Ac-PL3-Asp-Ser-B5-Asp-Glu-Ala-Ala-Phe-Hse-S8-Phe-BztA-Gln-NH2 | 1176 |
| I-1530 | Ac-PL3-Asp-Ser-B5-Asp-Glu-Ala-Ala-Phe-Hse-S8-Phe-BztA-Gln-NH2 | 1176 |
| I-1531 | Ac-PL3-Asp-Asn-B5-Asp-Glu-Ala-Ala-Phe-Gln-S8-Phe-BztA-Gln-NH2 | 1177 |
| I-1532 | Ac-PL3-Asp-Asn-B5-Asp-Glu-Ala-Ala-Phe-Gln-S8-Phe-BztA-Gln-NH2 | 1177 |
| I-1533 | Ac-PL3-Asp-Asn-B5-Asp-Glu-Ala-Ala-Phe-Leu-S8-Phe-BztA-Gln-NH2 | 1178 |
| I-1534 | Ac-PL3-Asp-Asn-B5-Asp-Glu-Ala-Ala-Phe-Hse-S8-Phe-BztA-Gln-NH2 | 1179 |
| I-1535 | Ac-PL3-Asp-Asn-B5-Asp-Glu-Ala-Ala-Phe-Hse-S8-Phe-BztA-Gln-NH2 | 1179 |
| I-1536 | Ac-PL3-Asp-Hleu-B5-Asp-Glu-Ala-Ala-Phe-Leu-S8-Phe-BztA-Gln-NH2 | 1180 |
| I-1537 | Ac-PL3-Asp-Hleu-B5-Asp-Glu-Ala-Ala-Phe-Hse-S8-Phe-BztA-Gln-NH2 | 1181 |
| I-1538 | Ac-PL3-Asp-Hleu-B5-Asp-Glu-Ala-Ala-Phe-Hse-S8-Phe-BztA-Gln-NH2 | 1181 |
| I-1539 | Ac-PL3-Asp-Gln-B5-Asp-Glu-Ala-Ala-Phe-Gln-S8-Phe-BztA-Gln-NH2 | 1182 |
| I-1540 | Ac-PL3-Asp-Gln-B5-Asp-Glu-Ala-Ala-Phe-Gln-S8-Phe-BztA-Gln-NH2 | 1182 |
| I-1541 | Ac-PL3-Asp-Gln-B5-Asp-Glu-Ala-Ala-Phe-Leu-S8-Phe-BztA-Gln-NH2 | 1183 |
| I-1542 | Ac-PL3-Asp-Gln-B5-Asp-Glu-Ala-Ala-Phe-Hse-S8-Phe-BztA-Gln-NH2 | 1184 |
| I-1543 | Ac-PL3-Asp-Gln-B5-Asp-Glu-Ala-Ala-Phe-Hse-S8-Phe-BztA-Gln-NH2 | 1184 |
| I-1544 | Ac-PL3-Asp-Hse-B5-Asp-Glu-Ala-Ala-Phe-Gln-S8-Phe-BztA-Gln-NH2 | 1185 |
| I-1545 | Ac-PL3-Asp-Hse-B5-Asp-Glu-Ala-Ala-Phe-Gln-S8-Phe-BztA-Gln-NH2 | 1185 |
| I-1546 | Ac-PL3-Asp-Hse-B5-Asp-Glu-Ala-Ala-Phe-Leu-S8-Phe-BztA-Gln-NH2 | 1186 |
| I-1547 | Ac-PL3-Asp-Hse-B5-Asp-Glu-Ala-Ala-Phe-Hse-S8-Phe-BztA-Gln-NH2 | 1187 |
| I-1548 | Ac-PL3-Asp-Hse-B5-Asp-Glu-Ala-Ala-Phe-Hse-S8-Phe-BztA-Gln-NH2 | 1187 |
| I-1549 | Ac-PL3-Asp-Phe-B5-Asp-Glu-Ala-Ala-Phe-Gln-S8-Phe-BztA-Gln-NH2 | 1188 |
| I-1550 | Ac-PL3-Asp-Phe-B5-Asp-Glu-Ala-Ala-Phe-Leu-S8-Phe-BztA-Gln-NH2 | 1189 |
| I-1551 | Ac-PL3-Asp-Phe-B5-Asp-Glu-Ala-Ala-Phe-Hse-S8-Phe-BztA-Gln-NH2 | 1190 |
| I-1552 | Ac-PL3-Asp-Lys-B5-Asp-Glu-Ala-Ala-Phe-Gln-S8-Phe-BztA-Gln-NH2 | 1191 |
| I-1553 | Ac-PL3-Asp-Lys-B5-Asp-Glu-Ala-Ala-Phe-Leu-S8-Phe-BztA-Gln-NH2 | 1192 |
| I-1554 | Ac-PL3-Asp-Lys-B5-Asp-Glu-Ala-Ala-Phe-Hse-S8-Phe-BztA-Gln-NH2 | 1193 |
| I-1555 | Ac-PL3-Asp-Npg-B5-Asn-Asn-Ala-Ala-Phe-Asn-PyrS2-3Thi-BztA-Gln-NH2 | 1194 |
| I-1556 | Ac-PL3-Asp-Ala-B5-Asn-Asn-Ala-Ala-Phe-Asn-PyrS2-3Thi-BztA-Gln-NH2 | 1195 |
| I-1557 | Ac-PL3-Asp-Npg-B5-Asn-Asn-Ala-Ala-Phe-Leu-PyrS2-3Thi-BztA-Gln-NH2 | 1196 |
| I-1558 | Ac-PL3-Asp-Npg-B5-Gln-Gln-Ala-Ala-Phe-Asn-PyrS2-3Thi-BztA-Gln-NH2 | 1197 |
| I-1559 | Ac-PL3-Asp-Npg-B5-Thr-Asn-Ala-Ala-Phe-Asn-PyrS2-3Thi-BztA-Gln-NH2 | 1198 |
| I-1560 | Ac-PL3-Asp-Npg-B5-Gln-Asn-Ala-Ala-Phe-Asn-PyrS2-3Thi-BztA-Gln-NH2 | 1199 |
| I-1561 | Ac-PL3-Asp-Npg-B5-Ser-Asn-Ala-Ala-Phe-Asn-PyrS2-3Thi-BztA-Gln-NH2 | 1200 |
| I-1562 | Ac-PL3-Asp-Npg-B5-Leu-Asn-Ala-Ala-Phe-Asn-PyrS2-3Thi-BztA-Gln-NH2 | 1201 |
| I-1563 | Ac-PL3-Asp-Npg-B5-Leu-Asn-Ala-Ala-Phe-Asn-PyrS2-3Thi-BztA-Gln-NH2 | 1201 |
| I-1564 | Ac-PL3-Asp-Npg-B5-Asn-Thr-Ala-Ala-Phe-Asn-PyrS2-3Thi-BztA-Gln-NH2 | 1202 |
| I-1565 | Ac-PL3-Asp-Npg-B5-Asn-Thr-Ala-Ala-Phe-Asn-PyrS2-3Thi-BztA-Gln-NH2 | 1202 |
| I-1566 | Ac-PL3-Asp-Npg-B5-Asn-Gln-Ala-Ala-Phe-Asn-PyrS2-3Thi-BztA-Gln-NH2 | 1203 |
| I-1567 | Ac-PL3-Asp-Npg-B5-Asn-Gln-Ala-Ala-Phe-Asn-PyrS2-3Thi-BztA-Gln-NH2 | 1203 |
| I-1568 | Ac-PL3-Asp-Npg-B5-Asn-Tyr-Ala-Ala-Phe-Asn-PyrS2-3Thi-BztA-Gln-NH2 | 1204 |
| I-1569 | Ac-PL3-Asp-Npg-B5-Asn-Tyr-Ala-Ala-Phe-Asn-PyrS2-3Thi-BztA-Gln-NH2 | 1204 |
| I-1570 | Ac-PL3-Asp-Npg-B5-Asn-Leu-Ala-Ala-Phe-Asn-PyrS2-3Thi-BztA-Gln-NH2 | 1205 |
| I-1571 | Ac-PL3-Asp-Npg-B5-Asp-[Pfb]GA-Ala-Ala-Phe-Asn-PyrS2-2C1F-BztA-Gln-NH2 | 1206 |
| I-1572 | Ac-PL3-Asp-Npg-B5-Asp-GA-Ala-Ala-Phe-Asn-PyrS2-2C1F-BztA-Gln-NH2 | 1207 |
| I-1573 | Ac-HypEs5-Asp-Npg-B5-Asp-Asp-Ala-Ala-Phe-Asn-PyrS2-3Thi-BztA-Gln-NH2 | 1208 |
| I-1574 | MeSO2-HypEs5-Asp-Npg-B5-Asp-Asp-Ala-Ala-Phe-Asn-PyrS2-3Thi-BztA-Gln-NH2 | 1209 |
| I-1575 | Ac-HypEs4-Asp-Npg-B5-Asp-Asp-Ala-Ala-Phe-Asn-PyrS2-3Thi-BztA-Gln-NH2 | 1210 |
| I-1576 | ProSAm3-Asp-Npg-B5-Asp-Asp-Ala-Ala-Phe-Asn-PyrS2-3Thi-BztA-Gln-NH2 | 1211 |
| I-1577 | ProSAm3-Asp-Npg-B5-Asp-Asp-Ala-Ala-Phe-Asn-PyrS2-3Thi-BztA-Gln-NH2 | 1211 |
| I-1578 | Ac-PL3-Asp-Npg-B5-Asp-3TzF-Ala-Ala-Phe-Asn-PyrS2-3Thi-BztA-Gln-NH2 | 1212 |
| I-1579 | Ac-PL3-Asp-Npg-B5-Asp-4TzF-Ala-Ala-Phe-Asn-PyrS2-3Thi-BztA-Gln-NH2 | 1213 |
| I-1580 | Ac-PL3-Asn-Phe-B5-Asp-Thr-iPrLys-Ala-Phe-Leu-PyrS2-Phe-BztA-Gln-NH2 | 1214 |
| I-1581 | Ac-PL3-Asn-Trp-B5-Asp-Thr-iPrLys-Ala-Phe-Leu-PyrS2-Phe-BztA-Gln-NH2 | 1215 |
| I-1582 | Ac-PL3-Asn-Leu-B5-Asp-Thr-iPrLys-Ala-Phe-Leu-PyrS2-Phe-BztA-Gln-NH2 | 1216 |
| I-1583 | Ac-PL3-Asn-Ser-B5-Asp-Thr-iPrLys-Ala-Phe-Leu-PyrS2-Phe-BztA-Gln-NH2 | 1217 |
| I-1584 | Ac-PL3-Asn-Gln-B5-Asp-Thr-iPrLys-Ala-Phe-Leu-PyrS2-Phe-BztA-Gln-NH2 | 1218 |
| I-1585 | Ac-PL3-Asn-Npg-B5-Asp-Thr-iPrLys-Ala-Phe-Leu-PyrS2-Phe-Tyr-Gln-NH2 | 1219 |
| I-1586 | Ac-PL3-Asn-Npg-B5-Asp-Thr-iPrLys-Ala-Phe-Leu-PyrS2-Phe-5C1W-Gln-NH2 | 1220 |
| I-1587 | Ac-PL3-Asn-Npg-B5-Asp-Thr-iPrLys-Ala-Phe-Leu-PyrS2-Phe-6C1W-Gln-NH2 | 1221 |
| I-1588 | Ac-PL3-Asn-Npg-B5-Asp-Thr-iPrLys-Ala-Phe-Leu-PyrS2-Phe-7FW-Gln-NH2 | 1222 |
| I-1589 | Ac-PL3-Asn-Npg-B5-Asp-Thr-iPrLys-Ala-Phe-Leu-PyrS2-Phe-1MeW-Gln-NH2 | 1223 |
| I-1590 | Ac-PL3-Asn-Npg-B5-Asp-Thr-iPrLys-Ala-Phe-Leu-PyrS2-Tyr-Trp-Gln-NH2 | 1224 |
| I-1591 | Ac-PL3-Asn-iPrLys-B5-Asp-Thr-iPrLys-Ala-Phe-Leu-PyrS2-Phe-BztA-Gln-NH2 | 1225 |
| I-1592 | Ac-PL3-Asn-Npg-B5-Asp-3cbmf-iPrLys-Ala-Phe-Leu-PyrS2-Phe-BztA-Gln-NH2 | 1226 |
| I-1593 | Ac-PL3-Asn-Npg-B5-Asp-Phe-iPrLys-Ala-Phe-Leu-PyrS2-Phe-BztA-Gln-NH2 | 1227 |
| I-1594 | Ac-PL3-Asn-Npg-B5-Asp-Npg-iPrLys-Ala-Phe-Leu-PyrS2-Phe-BztA-Gln-NH2 | 1228 |
| I-1595 | Ac-PL3-Asn-Npg-B5-Asp-3F3MeF-iPrLys-Ala-Phe-Leu-PyrS2-Phe-BztA-Gln-NH2 | 1229 |
| I-1596 | Ac-PL3-Asn-Npg-B5-Asp-Ala-iPrLys-Ala-Phe-Leu-PyrS2-Phe-BztA-Gln-NH2 | 1230 |
| I-1597 | Ac-PL3-Asn-Npg-B5-Asp-Thr-iPrLys-Ala-Ser-Leu-PyrS2-Phe-BztA-Gln-NH2 | 1231 |
| I-1598 | Ac-PL3-Asn-Npg-B5-Asp-Thr-iPrLys-Ala-Npg-Leu-PyrS2-Phe-BztA-Gln-NH2 | 1232 |
| I-1599 | Ac-PL3-Asn-Npg-B5-Asp-Thr-iPrLys-Ala-Phe-Phe-PyrS2-Phe-BztA-Gln-NH2 | 1233 |
| I-1600 | Ac-PL3-Asn-Npg-B5-Asp-Thr-iPrLys-Ala-Phe-Trp-PyrS2-Phe-BztA-Gln-NH2 | 1234 |
| I-1601 | Ac-PL3-Asn-Npg-B5-Asp-Thr-iPrLys-Ala-Phe-Ser-PyrS2-Phe-BztA-Gln-NH2 | 1235 |
| I-1602 | Ac-PL3-Asn-Npg-B5-Asp-Thr-iPrLys-Ala-Phe-Asn-PyrS2-Phe-BztA-Gln-NH2 | 1236 |
| I-1603 | Ac-PL3-Asn-Npg-B5-Asp-Thr-iPrLys-Ala-Phe-Leu-PyrS2-Npg-BztA-Gln-NH2 | 1237 |
| I-1604 | Ac-PL3-Asp-Npg-B5-Asp-3COOHF-Ala-Ala-Phe-Leu-PyrS2-2F3MeF-BztA-Gln-NH2 | 1116 |
| I-1605 | Ac-PL3-Asp-Npg-B5-Asp-TfeGA-Ala-Ala-Phe-Leu-PyrS2-3Thi-BztA-Gln-NH2 | 571 |
| I-1606 | Ac-PL3-Asp-Npg-B5-Asp-TfeGA-Ala-Ala-Phe-Thr-PyrS2-2C1F-BztA-Gln-NH2 | 639 |
| I-1607 | Ac-PL3-Asp-Npg-B5-Asp-TfeGA-Ala-Ala-Phe-Thr-PyrS2-2C1F-BztA-F2PipNva-NH2 | 1238 |
| I-1608 | Ac-PL3-Asp-Npg-B5-Asp-3COOHF-Ala-Ala-Phe-Leu-PyrS2-2F3MeF-BztA- | 1239 |
| | F2PipNva-NH2 | |
| I-1609 | Ac-PL3-Asp-Npg-B5-Asp-TfeGA-Ala-Ala-Phe-Thr-PyrS2-2C1F-BztA-MorphNva-NH2 | 1240 |
| I-1610 | Ac-PL3-Asp-Npg-B5-Asp-3COOHF-Ala-Ala-Phe-Leu-PyrS2-2F3MeF-BztA- | 1241 |
| | MorphNva-NH2 | |
| I-1611 | Ac-PL3-Asp-Npg-B5-Asp-TfeGA-Ala-Ala-Phe-Thr-PyrS2-2C1F-BztA-MePpzAsn-NH2 | 1242 |
| I-1612 | Ac-PL3-Asp-Npg-B5-Asp-3COOHF-Ala-Ala-Phe-Leu-PyrS2-2F3MeF-BztA- | 1243 |
| | MePpzAsn-NH2 | |
| I-1613 | Ac-PL3-Asp-Npg-B5-Asp-TfeGA-Ala-Ala-Phe-Thr-PyrS2-2C1F-BztA-MorphAsn-NH2 | 1244 |
| I-1614 | Ac-PL3-Asp-Npg-B5-Asp-3COOHF-Ala-Ala-Phe-Leu-PyrS2-2F3MeF-BztA- | 1245 |
| | MorphAsn-NH2 | |
| I-1615 | Ac-PL3-Asp-Npg-B5-Asp-TfeGA-Ala-Ala-Phe-Thr-PyrS2-2C1F-BztA-iPrLys-NH2 | 1246 |
| I-1616 | Ac-PL3-Asp-Npg-B5-Asp-3COOHF-Ala-Ala-Phe-Leu-PyrS2-2F3MeF-BztA-iPrLys- | 1247 |
| | NH2 | |
| I-1617 | Ac-PL3-Asp-Npg-B5-Asn-Thr-Ala-Ala-Phe-Asn-S8-3Thi-BztA-Gln-NH2 | 1248 |
| I-1618 | Ac-PL3-Asp-Npg-B5-Asn-Thr-Ala-Ala-Phe-Leu-PyrS2-3Thi-BztA-Gln-NH2 | 1249 |
| I-1619 | Ac-PL3-Asp-Npg-B5-Asn-Thr-Ala-Ala-Phe-nLeu-PyrS2-3Thi-BztA-Gln-NH2 | 1250 |
| I-1620 | Ac-PL3-Asp-Npg-B5-Asn-Thr-Ala-Ala-Phe-hLeu-PyrS2-3Thi-BztA-Gln-NH2 | 1251 |
| I-1621 | Ac-PL3-Asp-Npg-B5-Asn-Thr-Ala-Ala-Phe-Thr-PyrS2-3Thi-BztA-Gln-NH2 | 1252 |
| I-1622 | Ac-PL3-Asp-Npg-B5-Asp-[Et]GA-Ala-Ala-Phe-Asn-PyrS2-2C1F-BztA-Gln-NH2 | 1253 |
| I-1623 | Ac-PL3-Asp-Npg-B5-Asp-[sBu]GA-Ala-Ala-Phe-Asn-PyrS2-2C1F-BztA-Gln-NH2 | 1254 |
| I-1624 | Ac-PL3-Asp-Npg-B5-Asp-[Pfbn]GA-Ala-Ala-Phe-Asn-S8-3Thi-BztA-Gln-NH2 | 1255 |
| I-1625 | Ac-PL3-Asp-Npg-B5-Asp-[Tfp]GA-Ala-Ala-Phe-Asn-S8-3Thi-BztA-Gln-NH2 | 1256 |
| I-1626 | Ac-PL3-Asp-Npg-B5-Asp-[sBu]GA-Ala-Ala-Phe-Asn-S8-3Thi-BztA-Gln-NH2 | 1257 |
| I-1627 | Ac-PL3-Asp-Npg-B5-Asp-[Et]GA-Ala-Ala-Phe-Asn-S8-3Thi-BztA-Gln-NH2 | 1258 |
| I-1628 | Ac-PL3-Asp-Npg-B5-Asp-[Et]GA-Ala-Ala-Phe-Asn-S8-3Thi-BztA-Gln-NH2 | 1258 |
| I-1629 | Ac-PL3-Asp-Npg-B5-Asp-[Tfb]GA-Ala-Ala-Phe-Leu-PyrS2-3Thi-BztA-Gln-NH2 | 1259 |
| I-1630 | Ac-PL3-Asp-Npg-B5-Asp-[Bn]GA-Ala-Ala-Phe-Leu-PyrS2-3Thi-BztA-Gln-NH2 | 1260 |
| I-1631 | Ac-PL3-Asp-Npg-B5-Asp-[Me]GA-Ala-Ala-Phe-Leu-PyrS2-3Thi-BztA-Gln-NH2 | 1261 |
| I-1632 | Ac-PL3-Asp-Npg-B5-Asp-[Pfbn]GA-Ala-Ala-Phe-Leu-PyrS2-3Thi-BztA-Gln-NH2 | 1262 |
| I-1633 | Ac-PL3-Asp-Npg-B5-Asp-[Tfp]GA-Ala-Ala-Phe-Leu-PyrS2-3Thi-BztA-Gln-NH2 | 1263 |
| I-1634 | Ac-PL3-Asp-Npg-B5-Asp-[Tfb]GA-Ala-Ala-Phe-Asn-PyrS2-3Thi-BztA-Gln-NH2 | 1264 |
| I-1635 | Ac-PL3-Asp-Npg-B5-Asp-[Bn]GA-Ala-Ala-Phe-Asn-PyrS2-3Thi-BztA-Gln-NH2 | 1265 |
| I-1636 | Ac-PL3-Asp-Npg-B5-Asp-[Me]GA-Ala-Ala-Phe-Asn-PyrS2-3Thi-BztA-Gln-NH2 | 1266 |
| I-1637 | Ac-PL3-Asp-Npg-B5-Asp-[Me]GA-Ala-Ala-Phe-Asn-PyrS2-3Thi-BztA-Gln-NH2 | 1266 |
| I-1638 | Ac-PL3-Asp-Npg-B5-Asp-[Pfbn]GA-Ala-Ala-Phe-Asn-PyrS2-3Thi-BztA-Gln-NH2 | 1267 |
| I-1639 | Ac-PL3-Asp-Npg-B5-Asp-[Pfbn]GA-Ala-Ala-Phe-Asn-PyrS2-3Thi-BztA-Gln-NH2 | 1267 |
| I-1640 | Ac-PL3-Asp-Npg-B5-Asp-[Tfp]GA-Ala-Ala-Phe-Asn-PyrS2-3Thi-BztA-Gln-NH2 | 1268 |
| I-1641 | Ac-PL3-Asp-Npg-B5-Asp-TfeGA-Ala-Ala-Phe-Asn-PyrS2-3Thi-BztA-Gln-NEt2 | 1269 |
| I-1642 | Ac-PL3-Asp-Npg-B5-Asp-TfeGA-Ala-Ala-Phe-Asn-PyrS2-3Thi-BztA-Gln-NHCyHe | 1270 |
| I-1643 | Ac-PL3-Asn-Npg-B5-Asp-Gln-iPrLys-Ala-Phe-Leu-PyrS2-Phe-BztA-Lys-NH2 | 1271 |
| I-1644 | Ac-PL3-Asn-Npg-B5-Asp-Gln-iPrLys-Ala-Phe-Leu-PyrS2-Phe-BztA-Npg-NH2 | 1272 |
| I-1645 | Ac-PL3-Asn-Npg-B5-Asp-Gln-iPrLys-Ala-Phe-Leu-PyrS2-Phe-BztA-iPrLys-NH2 | 1273 |
| I-1646 | Ac-PL3-Asn-Npg-B5-Asp-Gln-iPrLys-Ala-Phe-Leu-PyrS2-Phe-BztA-Hse-NH2 | 1274 |
| I-1647 | Ac-PL3-Asn-Tyr-B5-Asp-Gln-iPrLys-Ala-Phe-Leu-PyrS2-Phe-BztA-Gln-NH2 | 1275 |
| I-1648 | Ac-PL3-Asn-Cha-B5-Asp-Gln-iPrLys-Ala-Phe-Leu-PyrS2-Phe-BztA-Gln-NH2 | 1276 |
| I-1649 | Ac-PL3-Asn-Npg-B5-Asp-Gln-iPrLys-Ala-Cha-Leu-PyrS2-Phe-BztA-Gln-NH2 | 1277 |
| I-1650 | Ac-PL3-Asn-Npg-B5-Asp-Gln-iPrLys-Ala-Phe-Cha-PyrS2-Phe-BztA-Gln-NH2 | 1278 |
| I-1651 | Ac-PL3-Asn-Npg-B5-Asp-Gln-iPrLys-Ala-Phe-Leu-PyrS2-Cha-BztA-Gln-NH2 | 1279 |
| I-1652 | Ac-PL3-Asn-Npg-B5-Asp-Gln-iPrLys-Ala-Phe-Leu-PyrS2-Phe-BztA-Cha-NH2 | 1280 |
| I-1653 | Ac-PL3-Asn-Npg-B5-Asp-Gln-iPrLys-Ala-Phe-iPrLys-PyrS2-Phe-BztA-Gln-NH2 | 1281 |
| I-1654 | Ac-PL3-Asn-Npg-B5-Asp-Gln-iPrLys-Ala-Phe-iPrLys-PyrS2-Phe-BztA-iPrLys-NH2 | 1282 |
| I-1655 | Ac-PL3-Asp-Npg-B5-Asp-2COOHF-Ala-Ala-Phe-Phe-PyrS2-2F3MeF-BztA-Gln-NH2 | 1283 |
| I-1656 | Ac-PL3-Asp-Npg-B5-Asp-2COOHF-Ala-Ala-Phe-Leu-PyrS2-2F3MeF-BztA-Gln-NH2 | 1284 |
| I-1657 | Ac-PL3-Asp-Npg-B5-Asp-2COOHF-Ala-Ala-Phe-Phe-PyrS2-2C1F-BztA-Gln-NH2 | 1285 |
| I-1658 | Ac-PL3-Asp-Npg-B5-Asp-2COOHF-Ala-Ala-Phe-Leu-PyrS2-2C1F-BztA-Gln-NH2 | 1286 |
| I-1659 | Ac-PL3-Asp-Npg-B5-Asp-2COOHF-Ala-Ala-Phe-Phe-PyrS2-2F3MeF-BztA-Ser-NH2 | 1287 |
| I-1660 | Ac-PL3-Asp-Npg-B5-Asp-2COOHF-Ala-Ala-Phe-Leu-PyrS2-2F3MeF-BztA-Ser-NH2 | 1288 |
| I-1661 | Ac-PL3-Asp-Npg-B5-Asp-2COOHF-Ala-Ala-Phe-Leu-PyrS2-2F3MeF-BztA-Ser-NH2 | 1288 |
| I-1662 | Ac-PL3-Asp-Npg-B5-Asp-2COOHF-Ala-Ala-Phe-Phe-PyrS2-2C1F-BztA-Ser-NH2 | 1289 |
| I-1663 | Ac-PL3-Asp-Npg-B5-Asp-2COOHF-Ala-Ala-Phe-Leu-PyrS2-2C1F-BztA-Ser-NH2 | 1290 |
| I-1664 | Ac-PL3-Asp-Npg-B5-Asp-2COOHF-Ala-Ala-Phe-Phe-PyrS2-3Thi-BztA-Ser-NH2 | 1291 |
| I-1665 | Ac-PL3-Asp-Npg-B5-Asp-2COOHF-Ala-Ala-Phe-Phe-PyrS2-3Thi-BztA-Ser-NH2 | 1291 |
| I-1666 | Ac-PL3-Asp-Npg-B5-Asp-2COOHF-Ala-Ala-Phe-Leu-PyrS2-3Thi-BztA-Ser-NH2 | 1292 |
| I-1667 | Ac-PL3-Asp-Npg-B5-Asp-2COOHF-Ala-Ala-Phe-Leu-PyrS2-3Thi-BztA-Ser-NH2 | 1292 |
| I-1668 | Ac-PL3-Asp-Npg-B5-Asp-2COOHF-Ala-Ala-Phe-Phe-PyrS2-3Thi-BztA-Thro1 | 1293 |
| I-1669 | Ac-PL3-Asp-Npg-B5-Asp-2COOHF-Ala-Ala-Phe-Phe-PyrS2-3Thi-BztA-Thro1 | 1293 |
| I-1670 | Ac-PL3-Asp-Npg-B5-Asp-2COOHF-Ala-Ala-Phe-Leu-PyrS2-3Thi-BztA-Thro1 | 1294 |
| I-1671 | Ac-PL3-Asp-Npg-B5-Asp-2COOHF-Ala-Ala-Phe-Leu-PyrS2-3Thi-BztA-Thro1 | 1294 |
| I-1672 | Ac-PL3-Asp-Npg-B5-Asp-3COOHF-Ala-Ala-Phe-Leu-PyrS2-2C1F-BztA-Gln-NHEt | 1295 |
| I-1673 | Ac-PL3-Asp-Npg-B5-Asp-3COOHF-Ala-Ala-Phe-Leu-PyrS2-2C1F-BztA-Gln-Alao1 | 1296 |
| I-1674 | Ac-PL3-Asp-Npg-B5-Asp-3COOHF-Ala-Ala-Phe-Leu-PyrS2-2C1F-BztA-Gln- | 1297 |
| I-1675 | Ac-PL3-Asp-Npg-B5-Asp-3COOHF-Ala-Ala-Phe-Leu-PyrS2-2C1F-BztA-Gln-Proo1 | 1298 |
| I-1676 | Ac-PL3-Asp-Npg-B5-Asp-3COOHF-Ala-Ala-Phe-Leu-PyrS2-2C1F-BztA-Gln-Leuo1 | 1299 |
| I-1677 | Ac-PL3-Asp-Npg-B5-Asp-3COOHF-Ala-Ala-Phe-Leu-PyrS2-2C1F-BztA-Gln-Thro1 | 1300 |
| I-1678 | Ac-PL3-Asp-Npg-B5-Asp-TfeGA-Ala-Ala-Phe-Leu-PyrS2-3Thi-BztA-Gln-NHEt | 1301 |
| I-1679 | Ac-PL3-Asp-Npg-B5-Asp-TfeGA-Ala-Ala-Phe-Leu-PyrS2-3Thi-BztA-Gln-Alao1 | 1302 |
| I-1680 | Ac-PL3-Asp-Npg-B5-Asp-TfeGA-Ala-Ala-Phe-Leu-PyrS2-3Thi-BztA-Gln-Pheo1 | 1303 |
| I-1681 | Ac-PL3-Asp-Npg-B5-Asp-TfeGA-Ala-Ala-Phe-Leu-PyrS2-3Thi-BztA-Gln-Proo1 | 1304 |
| I-1682 | Ac-PL3-Asp-Npg-B5-Asp-TfeGA-Ala-Ala-Phe-Leu-PyrS2-3Thi-BztA-Gln-Leuo1 | 1305 |
| I-1683 | Ac-PL3-Asp-Npg-B5-Asp-TfeGA-Ala-Ala-Phe-Leu-PyrS2-3Thi-BztA-Gln-Thro1 | 1306 |
| I-1684 | Ac-PL3-Asp-Npg-B5-Asp-3COOHF-Gly-Ala-Phe-Leu-PyrS2-2C1F-BztA-Gln-NH2 | 1307 |
| I-1685 | Ac-PL3-Asp-Npg-B5-Asp-3COOHF-nLeu-Ala-Phe-Leu-PyrS2-2C1F-BztA-Gln-NH2 | 1308 |
| I-1686 | Ac-PL3-Asp-Npg-B5-Asp-3COOHF-Aib-Ala-Phe-Leu-PyrS2-2C1F-BztA-Gln-NH2 | 1309 |
| I-1687 | Ac-PL3-Asp-Npg-B5-Asp-3COOHF-Ala-Gly-Phe-Leu-PyrS2-2C1F-BztA-Gln-NH2 | 1310 |
| I-1688 | Ac-PL3-Asp-Npg-B5-Asp-3COOHF-nLeu-Gly-Phe-Leu-PyrS2-2C1F-BztA-Gln-NH2 | 1311 |
| I-1689 | Ac-PL3-Asp-Npg-B5-Asp-3COOHF-Aib-Gly-Phe-Leu-PyrS2-2C1F-BztA-Gln-NH2 | 1312 |
| I-1690 | Ac-PL3-Asp-Npg-B5-Asp-TfeGA-Gly-Ala-Phe-Thr-PyrS2-2C1F-BztA-Gln-NH2 | 1313 |
| I-1691 | Ac-PL3-Asp-Npg-B5-Asp-TfeGA-Gly-Ala-Phe-Thr-PyrS2-2C1F-BztA-Gln-NH2 | 1313 |
| I-1692 | Ac-PL3-Asp-Npg-B5-Asp-TfeGA-nLeu-Ala-Phe-Thr-PyrS2-2C1F-BztA-Gln-NH2 | 1314 |
| I-1693 | Ac-PL3-Asp-Npg-B5-Asp-TfeGA-Aib-Ala-Phe-Thr-PyrS2-2C1F-BztA-Gln-NH2 | 1315 |
| I-1694 | Ac-PL3-Asp-Npg-B5-Asp-TfeGA-Ala-Gly-Phe-Thr-PyrS2-2C1F-BztA-Gln-NH2 | 1316 |
| I-1695 | Ac-PL3-Asp-Npg-B5-Asp-TfeGA-nLeu-Gly-Phe-Thr-PyrS2-2C1F-BztA-Gln-NH2 | 1317 |
| I-1696 | Ac-PL3-Asp-Npg-B5-Asp-TfeGA-Aib-Gly-Phe-Thr-PyrS2-2C1F-BztA-Gln-NH2 | 1318 |
| I-1697 | Ac-PL3-Asp-Npg-B5-Asp-TfeGA-Ala-Ala-Phe-Leu-PyrS3-3Thi-BztA-Gln-NH2 | 1319 |
| I-1698 | Ac-PL3-Asp-Npg-B5-Asp-TfeGA-Ala-Ala-Phe-Leu-S8-3Thi-BztA-Gln-NH2 | 1320 |
| I-1699 | Ac-PL3-Asp-Npg-B5-Asp-TfeGA-Ala-Ala-Phe-Hse-PyrS2-2C1F-BztA-Hse-NH2 | 684 |
| I-1700 | Ac-PL3-Asp-Npg-B5-Asp-TfeGA-Ala-Ala-Phe-Leu-PyrS2-Phe-BztA-Gln-NH2 | 1321 |
| I-1701 | Ac-PL3-Asp-Npg-B5-Asp-TfeGA-Ala-Ala-Phe-Leu-PyrS2-3PyrA-BztA-Gln-NH2 | 1322 |
| I-1702 | Ac-PL3-Asp-Npg-B5-Asp-TfeGA-Ala-Ala-Phe-Leu-PyrS2-2CNF-BztA-Gln-NH2 | 1323 |
| I-1703 | Ac-PL3-Asp-Npg-B5-Asp-TfeGA-Ala-Ala-Phe-Leu-PyrS2-CypA-BztA-Gln-NH2 | 1324 |
| I-1704 | Ac-PL3-Asp-Npg-B5-Asp-TfeGA-Ala-Ala-Phe-Leu-PyrS2-2F3MeF-BztA-Gln-NH2 | 1325 |
| I-1705 | Ac-PL3-Asp-Npg-B5-Asp-TfeGA-Ala-Ala-Phe-Leu-PyrS2-3Thi-BztA-Asn-NH2 | 1326 |
| I-1706 | Ac-PL3-Asp-Npg-B5-Asp-TfeGA-Ala-Ala-Phe-Leu-PyrS2-3Thi-BztA-Thr-NH2 | 1327 |
| I-1707 | Ac-PL3-Asp-Npg-B5-Asp-TfeGA-Ala-Ala-Phe-Leu-PyrS2-3Thi-BztA-2cbmf-NH2 | 1328 |
| I-1708 | Ac-PL3-Asp-Npg-B5-Asp-TfeGA-Ala-Ala-Phe-Leu-PyrS2-3Thi-BztA-3cbmf-NH2 | 1329 |
| I-1709 | Ac-PL3-Asp-Npg-B5-Asp-TfeGA-Ala-Ala-Phe-Leu-PyrS2-3Thi-BztA-AcLys-NH2 | 1330 |
| I-1710 | Ac-PL3-Asp-Npg-B5-Asp-TfeGA-Ala-Ala-Phe-Leu-PyrS2-3Thi-BztA-Ala-NH2 | 1331 |
| I-1711 | Ac-PL3-Asp-Npg-B5-Asp-TfeGA-Ala-Ala-Phe-Leu-PyrS2-Phe-BztA-Gln-NH2 | 1321 |
| I-1712 | Ac-PL3-Asp-Npg-B5-Asp-TfeGA-Ala-Ala-Phe-Leu-PyrS2-2CNF-BztA-Gln-NH2 | 1323 |
| I-1713 | Ac-PL3-Asp-Npg-B5-Asp-TfeGA-Ala-Ala-Phe-Leu-PyrS2-CypA-BztA-Gln-NH2 | 1324 |
| I-1714 | Ac-PL3-Asp-Npg-B5-Asp-TfeGA-Ala-Ala-Phe-Leu-PyrS2-2F3MeF-BztA-Gln-NH2 | 1325 |
| I-1715 | Ac-PL3-Asp-Npg-B5-Asp-TfeGA-Ala-Ala-Phe-Leu-PyrS2-3Thi-BztA-Asn-NH2 | 1326 |
| I-1716 | Ac-PL3-Asp-Npg-B5-Asp-TfeGA-Ala-Ala-Phe-Leu-PyrS2-3Thi-BztA-Thr-NH2 | 1327 |
| I-1717 | Ac-PL3-Asp-Npg-B5-Asp-TfeGA-Ala-Ala-Phe-Leu-PyrS2-3Thi-BztA-2cbmf-NH2 | 1328 |
| I-1718 | Ac-PL3-Asp-Npg-B5-Asp-TfeGA-Ala-Ala-Phe-Leu-PyrS2-3Thi-BztA-3cbmf-NH2 | 1329 |
| I-1719 | Ac-PL3-Asp-Npg-B5-Asp-TfeGA-Ala-Ala-Phe-Leu-PyrS2-3Thi-BztA-AcLys-NH2 | 1330 |
| I-1720 | Ac-PL3-Asp-Npg-B5-Asp-TfeGA-Ala-Ala-Phe-Leu-PyrS2-3Thi-BztA-Ala-NH2 | 1331 |
| I-1721 | Ac-PL3-AspSH-Npg-B5-Asp-Asp-Ala-Ala-Phe-Asn-PyrS2-2C1F-BztA-Gln-NH2 | 1332 |
| I-1722 | Ac-PL3-AspSH-Npg-B5-Asp-Asp-Ala-Ala-Phe-Asn-PyrS2-2C1F-BztA-Gln-NH2 | 1332 |
| I-1723 | Ac-PL3-[EtSSpy]AspE-Npg-B5-Asp-Asp-Ala-Ala-Phe-Asn-PyrS2-2C1F-BztA-Gln- | 1333 |
| | NH2 | |
| I-1724 | Ac-PL3-[EtSSpy]AspE-Npg-B5-Asp-Asp-Ala-Ala-Phe-Asn-PyrS2-2C1F-BztA-Gln- | 1333 |
| | NH2 | |
| I-1725 | Ac-PL3-Asp-Npg-B5-Asp-TfeGA-Ala-Ala-Phe-Leu-PyrS2-2MeF-BztA-Gln-NH2 | 1334 |
| I-1726 | Ac-PL3-Asp-Npg-B5-Asp-TfeGA-Ala-Ala-Phe-Leu-PyrS2-2MeF-BztA-Gln-NH2 | 1334 |
| I-1727 | Ac-PL3-Asp-Npg-B5-Asp-Asp-Ala-Ala-2NH2F-Asn-PyrS2-2C1F-BztA-Gln-NH2 | 1335 |
| I-1728 | Ac-PL3-Asp-Npg-B5-Asp-Asp-Ala-Ala-2NH2F-Asn-PyrS2-3Thi-BztA-Gln-NH2 | 1336 |
| I-1729 | Ac-PL3-Asp-Npg-B5-Asp-[Ac]GA-Ala-Ala-Phe-Leu-PyrS2-3Thi-BztA-Gln-NH2 | 1337 |
| I-1730 | Ac-PL3-Asp-Npg-B5-Asp-[CF3CO]GA-Ala-Ala-Phe-Leu-PyrS2-3Thi-BztA-Gln-NH2 | 1338 |
| I-1731 | Ac-PL3-Asp-Npg-B5-Asp-GA-Ala-Ala-Phe-Leu-PyrS2-3Thi-BztA-Gln-NH2 | 1339 |
| I-1732 | Ac-PL3-Asp-Npg-B5-Asp-GA-Ala-Ala-Phe-Asn-PyrS2-3Thi-BztA-Gln-NH2 | 1340 |
| I-1733 | Ac-PL3-Asp-Npg-B5-Asp-GA-Ala-Ala-Phe-Asn-PyrS2-3Thi-BztA-Gln-NH2 | 1340 |
| I-1734 | Ac-PL3-Asp-Npg-B5-Asp-[Pfb]GA-Ala-Ala-Phe-Asn-PyrS2-3Thi-BztA-Gln-NH2 | 1341 |
| I-1735 | Ac-PL3-Asp-Npg-B5-Asn-Thr-Ala-Ala-Phe-Asn-PyrS2-Phe-BztA-Gln-NH2 | 1342 |
| I-1736 | Ac-PL3-Asp-Npg-B5-Asn-Thr-Ala-Ala-Phe-Asn-PyrS2-2Thi-BztA-Gln-NH2 | 1343 |
| I-1737 | Ac-PL3-Asp-Npg-B5-Asn-Thr-Ala-Ala-Phe-Asn-PyrS2-2MeF-BztA-Gln-NH2 | 1344 |
| I-1738 | Ac-PL3-Asp-Npg-B5-Asn-Thr-Ala-Ala-Phe-Asn-PyrS2-2F3MeF-BztA-Gln-NH2 | 1345 |
| I-1739 | Ac-PL3-Asp-Npg-B5-Asn-Thr-Ala-Ala-Phe-Asn-PyrS2-3MeF-BztA-Gln-NH2 | 1346 |
| I-1740 | Ac-PL3-Asp-Npg-B5-Asn-Thr-Ala-Ala-Phe-Phe-PyrS2-3Thi-BztA-Gln-NH2 | 1347 |
| I-1741 | Ac-PL3-Asp-Npg-B5-Asn-Thr-Ala-Ala-Phe-Trp-PyrS2-3Thi-BztA-Gln-NH2 | 1348 |
| I-1742 | Ac-PL3-Asp-Npg-B5-Asn-Thr-Ala-Ala-Phe-Asn-PyrS2-3Thi-BztA-NH2 | 1349 |
| I-1743 | Ac-PL3-Asp-Npg-B5-Asn-Thr-Ala-Ala-Phe-Asn-PyrS2-3Thi-BztA-Arg-NH2 | 1350 |
| I-1744 | Ac-PL3-Asp-Npg-B5-Asn-Thr-Ala-Ala-Phe-Asn-PyrS2-3Thi-BztA-Phe-NH2 | 1351 |
| I-1745 | Ac-PL3-Asp-Npg-B5-Asn-Thr-Ala-Ala-Phe-Asn-PyrS2-3Thi-BztA-Thr-NH2 | 1352 |
| I-1746 | Ac-PL3-Asp-Npg-B5-Asn-Thr-Ala-Ala-Phe-Asn-PyrS2-3Thi-BztA-Asn-NH2 | 1353 |
| I-1747 | Ac-PL3-Asp-Npg-B5-Asp-TfeGA-Ala-Ala-Phe-Leu-PyrS2-3Thi-BztA-Leu-NH2 | 1354 |
| I-1748 | Ac-PL3-Asp-Npg-B5-Asp-TfeGA-Ala-Ala-Phe-Leu-PyrS2-3Thi-BztA-Leu-NH2 | 1354 |
| I-1749 | Ac-PL3-Asp-Npg-B5-Asp-TfeGA-Ala-Ala-Phe-Leu-PyrS2-3Thi-BztA-Phe-NH2 | 1355 |
| I-1750 | Ac-PL3-Asp-Npg-B5-Asp-TfeGA-Ala-Ala-Phe-Leu-PyrS2-3Thi-BztA-Phe-NH2 | 1355 |
| I-1751 | Ac-PL3-Asp-Npg-B5-Asp-TfeGA-Ala-Ala-Phe-Leu-PyrS2-3Thi-BztA-Val-NH2 | 1356 |
| I-1752 | Ac-PL3-Asp-Npg-B5-Asp-TfeGA-Ala-Ala-Phe-Leu-PyrS2-3Thi-BztA-Val-NH2 | 1356 |
| I-1753 | Ac-PL3-Asp-Npg-B5-Asp-TfeGA-Ala-Ala-Phe-Leu-PyrS2-3Thi-BztA-Leu-NH2 | 1354 |
| I-1754 | Ac-PL3-Asp-Npg-B5-Asp-TfeGA-Ala-Ala-Phe-Thr-PyrS2-3Thi-BztA-Leu-NH2 | 1357 |
| I-1755 | Ac-PL3-Asp-Npg-B5-Asp-TfeGA-Ala-Ala-Phe-Leu-PyrS2-3Thi-BztA-Phe-NH2 | 1355 |
| I-1756 | Ac-PL3-Asp-Npg-B5-Asp-TfeGA-Ala-Ala-Phe-Thr-PyrS2-3Thi-BztA-Phe-NH2 | 1358 |
| I-1757 | Ac-PL3-Asp-Npg-B5-Asp-TfeGA-Ala-Ala-Phe-Leu-PyrS2-3Thi-BztA-Val-NH2 | 1356 |
| I-1758 | Ac-PL3-Asp-Npg-B5-Asp-TfeGA-Ala-Ala-Phe-Thr-PyrS2-3Thi-BztA-Val-NH2 | 1359 |
| I-1759 | Ac-PL3-Asp-Npg-B5-Asp-TfeGA-iPrLys-Ala-Phe-Leu-PyrS2-3Thi-BztA-Leu-NH2 | 1360 |
| I-1760 | Ac-PL3-Asp-Npg-B5-Asp-TfeGA-iPrLys-Ala-Phe-Thr-PyrS2-3Thi-BztA-Leu-NH2 | 1361 |
| I-1761 | Ac-PL3-Asp-Npg-B5-Asp-TfeGA-iPrLys-Ala-Phe-Leu-PyrS2-3Thi-BztA-Phe-NH2 | 1362 |
| I-1762 | Ac-PL3-Asp-Npg-B5-Asp-TfeGA-iPrLys-Ala-Phe-Thr-PyrS2-3Thi-BztA-Phe-NH2 | 1363 |
| I-1763 | Ac-PL3-Asp-Npg-B5-Asp-TfeGA-iPrLys-Ala-Phe-Leu-PyrS2-3Thi-BztA-Val-NH2 | 1364 |
| I-1764 | Ac-PL3-Asp-Npg-B5-Asp-TfeGA-iPrLys-Ala-Phe-Thr-PyrS2-3Thi-BztA-Val-NH2 | 1365 |
| I-1765 | Ac-PL3-Asp-Npg-B5-Asp-TfeGA-iPrLys-Ala-Phe-Leu-PyrS2-3Thi-BztA-Leu-NH2 | 1360 |
| I-1766 | Ac-PL3-Asp-Npg-B5-Asp-TfeGA-iPrLys-Ala-Phe-Thr-PyrS2-3Thi-BztA-Leu-NH2 | 1361 |
| I-1767 | Ac-PL3-Asp-Npg-B5-Asp-TfeGA-iPrLys-Ala-Phe-Leu-PyrS2-3Thi-BztA-Phe-NH2 | 1362 |
| I-1768 | Ac-PL3-Asp-Npg-B5-Asp-TfeGA-iPrLys-Ala-Phe-Thr-PyrS2-3Thi-BztA-Phe-NH2 | 1363 |
| I-1769 | Ac-PL3-Asp-Npg-B5-Asp-TfeGA-iPrLys-Ala-Phe-Leu-PyrS2-3Thi-BztA-Val-NH2 | 1364 |
| I-1770 | Ac-PL3-Asp-Npg-B5-Asp-TfeGA-iPrLys-Ala-Phe-Thr-PyrS2-3Thi-BztA-Val-NH2 | 1365 |
| I-1771 | Ac-PL3-Asp-Npg-B5-bMe2Asp-TfeGA-Ala-Ala-Phe-Leu-PyrS2-3Thi-BztA-Gln-NH2 | 1366 |
| I-1772 | Ac-PL3-Asp-Npa-B5-Asp-TfeGA-Ala-Ala-Phe-Leu-PyrS2-3Thi-BztA-Gln-NH2 | 1367 |
| I-1773 | Ac-PL3-Asp-Npg-B5-Asp-TfeGA-Ala-Ala-Phe-Npg-PyrS2-3Thi-BztA-Gln-NH2 | 1368 |
| I-1774 | Ac-PL3-Asp-Npg-B5-Asp-TfeGA-Ala-Ala-Phe-Cha-PyrS2-3Thi-BztA-Gln-NH2 | 1369 |
| I-1775 | Ac-PL3-Asp-Cha-B5-Asp-TfeGA-Ala-Ala-Phe-Leu-PyrS2-3Thi-BztA-Gln-NH2 | 1370 |
| I-1776 | Ac-PL3-Asp-Npg-B5-Asp-TfeGA-Ala-Ala-Phe-nLeu-PyrS2-3Thi-BztA-Gln-NH2 | 1371 |
| I-1777 | Ac-PL3-Asp-nLeu-B5-Asp-TfeGA-Ala-Ala-Phe-Leu-PyrS2-3Thi-BztA-Gln-NH2 | 1372 |
| I-1778 | Ac-PL3-Asp-Npg-S5-Asp-Asp-Ala-Ala-Phe-R5-Ala-3Thi-BztA-S5-Ala-NH2 | 1373 |
| I-1779 | Ac-PL3-Asp-Npg-S5-Asp-Asp-Ala-Ala-Phe-R5-Ala-3Thi-BztA-S5-Ala-NH2 | 1373 |
| I-1780 | Ac-PL3-Asp-Npg-S5-Asp-Asp-Aib-Ala-Phe-R5-Ala-3Thi-BztA-S5-Ala-NH2 | 1374 |
| I-1781 | Ac-PL3-Asp-Npg-S5-Asp-Asp-Aib-Ala-Phe-R5-Ala-3Thi-BztA-S5-Ala-NH2 | 1374 |
| I-1782 | Ac-PL3-Asp-Npg-S5-Asp-Asp-R5-Ala-Phe-Leu-Ala-3Thi-BztA-PyrS2-Ala-NH2 | 1375 |
| I-1783 | Ac-PL3-Asp-Npg-B5-Asp-Tyr-Ala-Ala-Phe-Hse-PyrS2-3Thi-BztA-Gln-NH2 | 1376 |
| I-1784 | Ac-PL3-Asp-Npg-B5-Asp-Tyr-Ala-Ala-Phe-Leu-PyrS2-3Thi-BztA-Gln-NH2 | 1377 |
| I-1785 | Ac-PL3-Asp-Npg-B5-Asp-hTyr-Ala-Ala-Phe-Asn-PyrS2-3Thi-BztA-Gln-NH2 | 1378 |
| I-1786 | Ac-PL3-Asp-Npg-B5-Asp-hTyr-Ala-Ala-Phe-Hse-PyrS2-3Thi-BztA-Gln-NH2 | 1379 |
| I-1787 | Ac-PL3-Asp-Npg-B5-Asp-hTyr-Ala-Ala-Phe-Leu-PyrS2-3Thi-BztA-Gln-NH2 | 1380 |
| I-1788 | Ac-PL3-Asp-Npg-B5-Asp-Trp-Ala-Ala-Phe-Asn-PyrS2-3Thi-BztA-Gln-NH2 | 1381 |
| I-1789 | Ac-PL3-Asp-Npg-B5-Asp-Trp-Ala-Ala-Phe-Hse-PyrS2-3Thi-BztA-Gln-NH2 | 1382 |
| I-1790 | Ac-PL3-Asp-Npg-B5-Asp-Pff-Ala-Ala-Phe-Asn-PyrS2-3Thi-BztA-Gln-NH2 | 1383 |
| I-1791 | Ac-PL3-Asp-Npg-B5-Asp-Pff-Ala-Ala-Phe-Hse-PyrS2-3Thi-BztA-Gln-NH2 | 1384 |
| I-1792 | Ac-PL3-Asp-Npg-B5-Asp-Pff-Ala-Ala-Phe-Leu-PyrS2-3Thi-BztA-Gln-NH2 | 1385 |
| I-1793 | Ac-PL3-Asp-Npg-B5-Asp-His-Ala-Ala-Phe-Hse-PyrS2-3Thi-BztA-Gln-NH2 | 1386 |
| I-1794 | Ac-PL3-Asp-Npg-B5-Asp-His-Ala-Ala-Phe-Hse-PyrS2-3Thi-BztA-Gln-NH2 | 1386 |
| I-1795 | Ac-PL3-Asp-Npg-B5-Asp-His-Ala-Ala-Phe-Leu-PyrS2-3Thi-BztA-Gln-NH2 | 1387 |
| I-1796 | Ac-PL3-Asp-Npg-B5-Asp-His-Ala-Ala-Phe-Leu-PyrS2-3Thi-BztA-Gln-NH2 | 1387 |
| I-1797 | Ac-PL3-Asp-Npg-B5-Asp-TfeGA-Ala-Ala-Phe-Leu-PyrS2-3Thi-BztA-[4Pippip]GlnR- | 1388 |
| | NH2 | |
| I-1798 | Ac-PL3-Asp-Npg-B5-Asp-TfeGA-Ala-Ala-Phe-Leu-PyrS2-3Thi-BztA-[4Pippip]GlnR- | 1388 |
| | NH2 | |
| I-1799 | Ac-PL3-Asp-Npg-B5-Asp-TfeGA-Ala-Ala-Phe-Leu-PyrS2-3Thi-BztA- | 1389 |
| | [Me2NPrPip]GlnR-NH2 | |
| I-1800 | Ac-PL3-Asp-Npg-B5-Asp-TfeGA-Ala-Ala-Phe-Leu-PyrS2-3Thi-BztA- | 1389 |
| | [Me2NPrPip]GlnR-NH2 | |
| I-1801 | Ac-PL3-Asp-Npg-B5-Asp-TfeGA-Ala-Ala-Phe-Leu-PyrS2-3Thi-BztA- | 1390 |
| | [4MePpzPip]GlnR-NH2 | |
| I-1802 | Ac-PL3-Asp-Npg-B5-Asp-TfeGA-Ala-Ala-Phe-Leu-PyrS2-3Thi-BztA- | 1390 |
| | [4MePpzPip]GlnR-NH2 | |
| I-1803 | Ac-PL3-Asp-Npg-B5-Asp-TfeGA-Ala-Ala-Phe-Leu-PyrS2-3Thi-BztA-[TfePpz]GlnR- | 1391 |
| | NH2 | |
| I-1804 | Ac-PL3-Asp-Npg-B5-Asp-TfeGA-Ala-Ala-Phe-Leu-PyrS2-3Thi-BztA- | 1392 |
| | [RDMAPyr]GlnR-NH2 | |
| I-1805 | Ac-PL3-Asp-Npg-B5-Asp-TfeGA-Ala-Ala-Phe-Leu-PyrS2-3Thi-BztA- | 1392 |
| | [RDMAPyr]GlnR-NH2 | |
| I-1806 | Ac-PL3-Asp-Npg-S5-Asp-Asp-Ala-Ala-Phe-Leu-Aib-3Thi-BztA-Gln-NH2 | 1393 |
| I-1807 | Ac-PL3-Asp-Npg-S5-Asp-Asp-Ala-Ala-Phe-Leu-Aib-3Thi-BztA-Gln-NH2 | 1393 |
| I-1808 | Ac-PL3-Asp-Npg-B5-Asp-Asp-Ala-Ala-Phe-Leu-PyrS2-3Thi-BztA-Gln-NH2 | 528 |
| I-1809 | Ac-PL3-Asp-Npg-S5-Asp-Asp-Ala-Ala-Phe-Leu-Aib-3Thi-BztA-Gln-NH2 | 1393 |
| I-1810 | Ac-Pro-Asp-Npg-R5-Asp-Asp-Ala-Ala-Phe-Leu-PyrS2-3Thi-BztA-Gln-NH2 | 856 |
| I-1811 | Ac-PL3-Asp-Leu-B5-Asp-Asp-Ala-Ala-Phe-Leu-PyrS2-Phe-Trp-Gln-NH2 | 1394 |
| I-1812 | Ac-PL3-Asp-Npg-B5-Asp-Asp-Ala-Ala-Phe-Asn-PyrS1-3Thi-BztA-Gln-NH2 | 1395 |
| I-1813 | Ac-PL3-Asp-Npg-B5-Asp-Asp-Ala-Ala-Phe-Asn-PyrS1-3Thi-BztA-Gln-NH2 | 1395 |
| I-1814 | Ac-PL3-Asp-Npg-B5-Asp-Asp-Ala-Ala-Phe-Leu-PyrS1-3Thi-BztA-Gln-NH2 | 1396 |
| I-1815 | Ac-PL3-Asp-Npg-B5-Asp-Asp-Ala-Ala-Phe-Leu-PyrS1-3Thi-BztA-Gln-NH2 | 1396 |
| I-1816 | Ac-PL3-Asp-Npg-B5-Asp-Asp-Ala-Ala-Phe-Asn-PyrS-3Thi-BztA-Gln-NH2 | 1397 |
| I-1817 | Ac-PL3-Asp-Npg-B5-Asp-Asp-Ala-Ala-Phe-Asn-PyrS-3Thi-BztA-Gln-NH2 | 1397 |
| I-1818 | Ac-PL3-Asp-Npg-B5-Asp-Asp-Ala-Ala-Phe-Leu-PyrS-3Thi-BztA-Gln-NH2 | 1398 |
| I-1819 | Ac-PL3-Asp-Npg-B5-Asp-Asp-Ala-Ala-Phe-Asn-S7-3Thi-BztA-Gln-NH2 | 1399 |
| I-1820 | Ac-PL3-Asp-Npg-B5-Asp-Asp-Ala-Ala-Phe-Leu-S7-3Thi-BztA-Gln-NH2 | 1400 |
| I-1821 | Ac-PL3-Asp-Npg-B5-Asp-Asp-Ala-Ala-Phe-Asn-PyrS1-3Thi-BztA-Gln-NH2 | 1395 |
| I-1822 | Ac-PL3-Asp-Npg-B5-Asp-Asp-Ala-Ala-Phe-Leu-PyrS1-3Thi-BztA-Gln-NH2 | 1396 |
| I-1823 | Ac-PL3-Asp-Npg-B5-Asp-Asp-Ala-Ala-Phe-Asn-PyrS-3Thi-BztA-Gln-NH2 | 1397 |
| I-1824 | Ac-PL3-Asp-Npg-B5-Asp-Asp-Ala-Ala-Phe-Leu-PyrS-3Thi-BztA-Gln-NH2 | 1398 |
| I-1825 | Ac-PL3-Asp-Npg-B5-Asp-Asp-Ala-Ala-Phe-Asn-S7-3Thi-BztA-Gln-NH2 | 1399 |
| I-1826 | Ac-PL3-Asp-Npg-B5-Asp-Asp-Ala-Ala-Phe-Asn-Az-3Thi-BztA-Gln-NH2 | 1401 |
| I-1827 | Ac-PL3-Asp-Npg-B5-Asp-Asp-Ala-Ala-Phe-Asn-Az-3Thi-BztA-Gln-NH2 | 1401 |
| I-1828 | Ac-PL3-Asp-Npg-B5-Asp-Asp-Ala-Ala-Phe-Leu-Az-3Thi-BztA-Gln-NH2 | 1402 |
| I-1829 | Ac-PL3-Asp-Npg-B5-Asp-Asp-Ala-Ala-Phe-Asn-SdN-3Thi-BztA-Gln-NH2 | 1403 |
| I-1830 | Ac-PL3-Asp-Npg-B5-Asp-Asp-Ala-Ala-Phe-Leu-SdN-3Thi-BztA-Gln-NH2 | 1404 |
| I-1831 | Ac-PL3-Asp-Npg-B5-Asp-Asp-Ala-Ala-Phe-Asn-Az-3Thi-BztA-Gln-NH2 | 1401 |
| I-1832 | Ac-PL3-Asp-Npg-B5-Asp-Asp-Ala-Ala-Phe-Leu-Az-3Thi-BztA-Gln-NH2 | 1402 |
| I-1833 | Ac-PL3-Asp-Npg-B5-Asp-TfeGA-Ala-Ala-Phe-Leu-PyrS2-2C1F-BztA-Gln-NH2 | 1405 |
| I-1834 | Ac-PL3-Asp-Npg-B5-Asp-[Bn]GAbu-Ala-Ala-Phe-Leu-PyrS2-3Thi-BztA-Gln-NH2 | 1406 |
| I-1835 | Ac-PL3-Asp-Npg-B5-Asp-[Et]GAbu-Ala-Ala-Phe-Leu-PyrS2-3Thi-BztA-Gln-NH2 | 1407 |
| I-1836 | Ac-PL3-Asp-Npg-B5-Asp-[CH2CO2H]GAbu-Ala-Ala-Phe-Leu-PyrS2-3Thi-BztA-Gln- | 1408 |
| | NH2 | |
| I-1837 | Ac-PL3-Asp-Npg-B5-Asp-[Tfb]GAbu-Ala-Ala-Phe-Leu-PyrS2-3Thi-BztA-Gln-NH2 | 1409 |
| I-1838 | Ac-PL3-Asp-Npg-B5-Asp-[Pfbn]GAbu-Ala-Ala-Phe-Leu-PyrS2-3Thi-BztA-Gln-NH2 | 1410 |
| I-1839 | Ac-PL3-Asp-Npg-B5-Asp-[Tfp]GAbu-Ala-Ala-Phe-Leu-PyrS2-3Thi-BztA-Gln-NH2 | 1411 |
| I-1840 | Ac-PL3-Asp-Npg-B5-Asp-[Ac]GAbu-Ala-Ala-Phe-Leu-PyrS2-3Thi-BztA-Gln-NH2 | 1412 |
| I-1841 | Ac-PL3-Asp-Npg-B5-Asp-GAbu-Ala-Ala-Phe-Leu-PyrS2-3Thi-BztA-Gln-NH2 | 1413 |
| I-1842 | Ac-PL3-Asp-Npg-B5-Asp-[MorphEt]GAbu-Ala-Ala-Phe-Leu-PyrS2-3Thi-BztA-Gln- | 1414 |
| | NH2 | |
| I-1843 | Ac-PL3-Asp-Npg-B5-Asp-[2COOH4NO2Ph]Dap-Ala-Ala-Phe-Leu-PyrS2-3Thi-BztA- | 1415 |
| | Gln-NH2 | |
| I-1844 | Ac-PL3-Asp-Npg-B5-Asp-[3COOH4NO2Ph]Dap-Ala-Ala-Phe-Leu-PyrS2-3Thi-BztA- | 1416 |
| | Gln-NH2 | |
| I-1845 | Ac-PL3-Asp-Npg-B5-Asp-[2Nic]Dap-Ala-Ala-Phe-Leu-PyrS2-3Thi-BztA-Gln-NH2 | 1417 |
| I-1846 | Ac-HyPEs5-Asp-Npg-B5-Asp-TfeGA-Ala-Ala-Phe-Thr-PyrS2-2C1F-BztA-Gln-NH2 | 1418 |
| I-1847 | Ac-HypEs5-Asp-Npg-B5-Asp-3COOHF-Ala-Ala-Phe-Leu-PyrS2-2C1F-BztA-Gln-NH2 | 1419 |
| I-1848 | Ac-PL3-Asp-Npg-B5-Asp-[2COOH4NO2Ph]Cys-Ala-Ala-Phe-Leu-PyrS2-3Thi-BztA- | 1420 |
| | Gln-NH2 | |
| I-1849 | Ac-PL3-Asp-Npg-B5-Asp-3COOHF-Ala-Ala-Phe-Ala-PyrS2-2C1F-BztA-Ser-NH2 | 1421 |
| I-1850 | Ac-PL3-Asp-Npg-B5-Asp-3COOHF-Ala-Ala-Phe-Val-PyrS2-2C1F-BztA-Ser-NH2 | 1422 |
| I-1851 | Ac-PL3-Asp-Npg-B5-Asp-3COOHF-Ala-Ala-Phe-Leu-PyrS2-2C1F-BztA-Ala-NH2 | 1423 |
| I-1852 | Ac-PL3-Asp-Npg-B5-Asp-3COOHF-Ala-Ala-Phe-Leu-PyrS2-2C1F-BztA-NH2 | 1424 |
| I-1853 | Ac-PL3-Asn-Npg-B5-Asp-3COOHF-Ala-Ala-Phe-Leu-PyrS2-2C1F-BztA-Ser-NH2 | 1425 |
| I-1854 | Ac-PL3-Asp-Npg-B5-Asp-TfeGA-Ala-Ala-[CH2NMe2]TriAzDap-Leu-PyrS2-3Thi- | 1426 |
| | BztA-Gln-NH2 | |
| I-1855 | Ac-PL3-Asp-Npg-B5-Asp-TfeGA-Ala-Ala-[CH2NMe2]TriAzDap-Leu-PyrS2-3Thi- | 1426 |
| | BztA-Gln-NH2 | |
| I-1856 | Ac-PL3-Asp-Npg-B5-Asp-TfeGA-Ala-Ala-[CH2Ppz]TriAzDap-Leu-PyrS2-3Thi-BztA- | 1427 |
| | Gln-NH2 | |
| I-1857 | Ac-PL3-Asp-Npg-B5-Asp-TfeGA-Ala-Ala-[CH2Ppz]TriAzDap-Leu-PyrS2-3Thi-BztA- | 1427 |
| | Gln-NH2 | |
| I-1858 | Ac-PL3-Asp-Npg-B5-Asp-TfeGA-Ala-Ala-[MorphCH2]TriAzDap-Leu-PyrS2-3Thi- | 1428 |
| | BztA-Gln-NH2 | |
| I-1859 | Ac-PL3-Asp-Npg-B5-Asp-TfeGA-Ala-Ala-[MorphCH2]TriAzDap-Leu-PyrS2-3Thi- | 1428 |
| | BztA-Gln-NH2 | |
| I-1860 | Ac-PL3-Asp-Npg-B5-Asp-TfeGA-Ala-Ala-[SO2MorphCH2]TriAzDap-Leu-PyrS2- | 1429 |
| | 3Thi-BztA-Gln-NH2 | |
| I-1861 | Ac-PL3-Asp-Npg-B5-Asp-TfeGA-Ala-Ala-Phe-[CH2NMe2]TriAzDap-PyrS2-3Thi- | 1430 |
| | BztA-Gln-NH2 | |
| I-1862 | Ac-PL3-Asp-Npg-B5-Asp-TfeGA-Ala-Ala-Phe-[CH2NMe2]TriAzDap-PyrS2-3Thi- | 1430 |
| | BztA-Gln-NH2 | |
| I-1863 | Ac-PL3-Asp-Npg-B5-Asp-TfeGA-Ala-Ala-Phe-[CH2Ppz]TriAzDap-PyrS2-3Thi-BztA- | 1431 |
| | Gln-NH2 | |
| I-1864 | Ac-PL3-Asp-Npg-B5-Asp-TfeGA-Ala-Ala-Phe-[CH2Ppz]TriAzDap-PyrS2-3Thi-BztA- | 1431 |
| | Gln-NH2 | |
| I-1865 | Ac-PL3-Asp-Npg-B5-Asp-TfeGA-Ala-Ala-Phe-[MorphCH2]TriAzDap-PyrS2-3Thi- | 1432 |
| | BztA-Gln-NH2 | |
| I-1866 | Ac-PL3-Asp-Npg-B5-Asp-TfeGA-Ala-Ala-Phe-[MorphCH2]TriAzDap-PyrS2-3Thi- | 1432 |
| | BztA-Gln-NH2 | |
| I-1867 | Ac-PL3-Asp-Npg-B5-Asp-TfeGA-Ala-Ala-Phe-[MorphCH2]TriAzDap-PyrS2-3Thi- | 1432 |
| | BztA-Gln-NH2 | |
| I-1868 | Ac-PL3-Asp-Npg-B5-Asp-TfeGA-Ala-Ala-Phe-[SO2MorphCH2]TriAzDap-PyrS2- | 1433 |
| | 3Thi-BztA-Gln-NH2 | |
| I-1869 | Ac-PL3-Asp-Npg-B5-Asp-TfeGA-Ala-Ala-Phe-[SO2MorphCH2]TriAzDap-PyrS2- | 1433 |
| | 3Thi-BztA-Gln-NH2 | |
| I-1870 | Ac-PL3-Asp-Npg-B5-Asn-Thr-Ala-Ala-Phe-Asn-PyrS2-3Thi-1NapA-Gln-NH2 | 1434 |
| I-1871 | Ac-PL3-Asp-Npg-B5-Asn-Thr-Ala-Ala-Phe-Asn-PyrS2-3Thi-1NapA-Gln-NH2 | 1434 |
| I-1872 | Ac-PL3-Asp-Npg-B5-Asn-Thr-Ala-Ala-Phe-Asn-PyrS2-3Thi-2NapA-Gln-NH2 | 1435 |
| I-1873 | Ac-PL3-Asp-Npg-B5-Asn-Thr-Ala-Ala-Phe-Asn-PyrS2-3Thi-2NapA-Gln-NH2 | 1435 |
| I-1874 | Ac-PL3-Asp-Npg-B5-Asn-Thr-Ala-Ala-Phe-Asn-PyrS2-3Thi-Cha-Gln-NH2 | 1436 |
| I-1875 | Ac-PL3-Asp-Npg-B5-Asn-Thr-Ala-Ala-Phe-Asn-PyrS2-3Thi-Cha-Gln-NH2 | 1436 |
| I-1876 | Ac-PL3-Asp-Npg-B5-Asn-Thr-Ala-Ala-Phe-Asn-PyrS2-3Thi-Trp-Gln-NH2 | 1437 |
| I-1877 | Ac-PL3-Asp-Npg-B5-Asn-Thr-Ala-Ala-Phe-Asn-PyrS2-3Thi-Trp-Gln-NH2 | 1437 |
| I-1878 | Ac-PL3-Asp-Npg-B5-Asn-Thr-Ala-Ala-Phe-Asn-PyrS2-3Thi-BztA-Val-NH2 | 1438 |
| I-1879 | Ac-PL3-Asp-Npg-B5-Asn-Thr-Ala-Ala-Phe-Asn-PyrS2-3Thi-BztA-Val-NH2 | 1438 |
| I-1880 | Ac-PL3-Asp-Npg-B5-Asn-Thr-Ala-Ala-Phe-Asn-PyrS2-3Thi-BztA-Leu-NH2 | 1439 |
| I-1881 | Ac-PL3-Asp-Npg-B5-Asn-Thr-Ala-Ala-Phe-Asn-PyrS2-3Thi-BztA-Leu-NH2 | 1439 |
| I-1882 | Ac-PL3-Asp-Npg-B5-Asn-Thr-Ala-Ala-Phe-Arg-PyrS2-3Thi-BztA-Gln-NH2 | 1440 |
| I-1883 | Ac-PL3-Asp-Npg-B5-Asn-Thr-Ala-Ala-Phe-Arg-PyrS2-3Thi-BztA-Gln-NH2 | 1440 |
| I-1884 | Ac-PL3-Asp-Cha-B5-Asn-Thr-Ala-Ala-Phe-Asn-PyrS2-3Thi-BztA-Gln-NH2 | 1441 |
| I-1885 | Ac-PL3-Asp-Cha-B5-Asn-Thr-Ala-Ala-Phe-Asn-PyrS2-3Thi-BztA-Gln-NH2 | 1441 |
| I-1886 | Ac-PL3-Asp-Npg-B5-Arg-Asp-Ala-Ala-Phe-Asn-PyrS2-3Thi-BztA-Gln-NH2 | 1442 |
| I-1887 | Ac-PL3-Asp-Npg-B5-Arg-Asp-Ala-Ala-Phe-Asn-PyrS2-3Thi-BztA-Gln-NH2 | 1442 |
| I-1888 | Ac-PL3-Asp-Npg-B5-Arg-Thr-Ala-Ala-Phe-Asn-PyrS2-3Thi-BztA-Gln-NH2 | 1443 |
| I-1889 | Ac-PL3-Asp-Npg-B5-Arg-Thr-Ala-Ala-Phe-Asn-PyrS2-3Thi-BztA-Gln-NH2 | 1443 |
| I-1890 | Ac-PL3-Asp-Npg-B5-Asp-Arg-Ala-Ala-Phe-Asn-PyrS2-3Thi-BztA-Gln-NH2 | 1444 |
| I-1891 | Ac-PL3-Asp-Npg-B5-Asp-Arg-Ala-Ala-Phe-Asn-PyrS2-3Thi-BztA-Gln-NH2 | 1444 |
| I-1892 | Ac-PL3-Asp-Npg-B5-Asn-Arg-Ala-Ala-Phe-Asn-PyrS2-3Thi-BztA-Gln-NH2 | 1445 |
| I-1893 | Ac-PL3-Asp-Npg-B5-Asn-Arg-Ala-Ala-Phe-Asn-PyrS2-3Thi-BztA-Gln-NH2 | 1445 |
| I-1894 | Ac-PL3-Asp-Npg-B5-Asp-3COOHF-Ala-Ala-CypA-Leu-PyrS2-2C1F-BztA-Ser-NH2 | 1446 |
| I-1895 | Ac-PL3-Asp-Npg-B5-Asp-3COOHF-Ala-Ala-Cba-Leu-PyrS2-2C1F-BztA-Ser-NH2 | 1447 |
| I-1896 | Ac-PL3-Asp-Npg-B5-Asp-3COOHF-Ala-Ala-Phe-Leu-PyrS2-CypA-BztA-Ser-NH2 | 1448 |
| I-1897 | Ac-PL3-Asp-Npg-B5-Asp-3COOHF-Ala-Ala-Phe-Leu-PyrS2-Cba-BztA-Ser-NH2 | 1449 |
| I-1898 | Ac-PL3-Asp-Npg-B5-Asp-3COOHF-Ala-Ala-Phe-Leu-PyrS2-Cba-BztA-Ser-NH2 | 1449 |
| I-1899 | Ac-PL3-Asp-Npg-B5-Asp-3COOHF-Aib-Ala-Phe-Leu-PyrS2-2C1F-BztA-Ser-NH2 | 1450 |
| I-1900 | Ac-PL3-Asp-Npg-B5-Asp-3COOHF-Aib-Ala-Phe-Leu-PyrS2-2C1F-BztA-Ser-NH2 | 1450 |
| I-1901 | Ac-PL3-Asp-Npg-B5-Asp-3COOHF-Ser-Ala-Phe-Leu-PyrS2-2C1F-BztA-Ser-NH2 | 1451 |
| I-1902 | Ac-PL3-Asp-Npg-B5-Asp-3COOHF-Ser-Ala-Phe-Leu-PyrS2-2C1F-BztA-Ser-NH2 | 1451 |
| I-1903 | Ac-PL3-Asp-Npg-B5-Asp-3COOHF-Ala-Ala-Phe-Ser-PyrS2-2C1F-BztA-Ser-NH2 | 1452 |
| I-1904 | Ac-PL3-Asp-Npg-B5-Asp-3COOHF-Ala-Ala-Phe-Leu-PyrS2-2C1F-BztA-Asn-NH2 | 1453 |
| I-1905 | Ac-PL3-Asp-Npg-B5-Asp-3COOHF-Ala-Ala-Phe-Leu-PyrS2-2C1F-BztA-Ser-Thro1 | 1454 |
| I-1906 | Ac-PL3-Asp-Npg-B5-Asp-3COOHF-Ala-Ala-Phe-Leu-PyrS2-2C1F-BztA-Ser-Prool | 1455 |
| I-1907 | Ac-PL3-Asp-Npg-B5-Asp-3COOHF-Ala-Ala-Phe-Leu-PyrS2-2C1F-BztA-Ser-Alao1 | 1456 |
| I-1908 | Ac-PL3-Asp-Npg-B5-Asp-3COOHF-Ala-Ala-Phe-Leu-PyrS2-2C1F-BztA-Ser-NHEt | 1457 |
| I-1909 | Ac-PL3-Asp-Npg-B5-Asp-3COOHF-iPrLys-Ala-Phe-Leu-PyrS2-3Thi-BztA-Phe-NH2 | 1458 |
| I-1910 | Ac-PL3-Asp-Npg-B5-Asp-3COOHF-His-Ala-Phe-Leu-PyrS2-3Thi-BztA-Phe-NH2 | 1459 |
| I-1911 | Ac-PL3-Asp-Npg-B5-Asp-3COOHF-Lys-Ala-Phe-Leu-PyrS2-3Thi-BztA-Phe-NH2 | 1460 |
| I-1912 | Ac-PL3-Asp-Npg-B5-Asp-3COOHF-Gln-Ala-Phe-Leu-PyrS2-3Thi-BztA-Phe-NH2 | 1461 |
| I-1913 | Ac-PL3-Asp-Npg-B5-Asp-3COOHF-nLeu-Ala-Phe-Leu-PyrS2-3Thi-BztA-Phe-NH2 | 1462 |
| I-1914 | Ac-PL3-Asp-Npg-B5-Asp-3COOHF-Aib-Ala-Phe-Leu-PyrS2-3Thi-BztA-Phe-NH2 | 1463 |
| I-1915 | Ac-PL3-Asp-Npg-B5-Asp-3COOHF-iPrLys-Ala-Phe-Leu-PyrS2-3Thi-BztA-Ala-NH2 | 1464 |
| I-1916 | Ac-PL3-Asp-Npg-B5-Asp-3COOHF-His-Ala-Phe-Leu-PyrS2-3Thi-BztA-Ala-NH2 | 1465 |
| I-1917 | Ac-PL3-Asp-Npg-B5-Asp-3COOHF-Lys-Ala-Phe-Leu-PyrS2-3Thi-BztA-Ala-NH2 | 1466 |
| I-1918 | Ac-PL3-Asp-Npg-B5-Asp-3COOHF-Gln-Ala-Phe-Leu-PyrS2-3Thi-BztA-Ala-NH2 | 1467 |
| I-1919 | Ac-PL3-Asp-Npg-B5-Asp-3COOHF-nLeu-Ala-Phe-Leu-PyrS2-3Thi-BztA-Ala-NH2 | 1468 |
| I-1920 | Ac-PL3-Asp-Npg-B5-Asp-3COOHF-Aib-Ala-Phe-Leu-PyrS2-3Thi-BztA-Ala-NH2 | 1469 |
| I-1921 | Ac-PL3-Asp-Npg-B5-Asp-S3COOPipA-Ala-Ala-Phe-Leu-PyrS2-3Thi-BztA-Gln-NH2 | 1470 |
| I-1922 | Ac-PL3-Asn-Cha-B5-Asp-Gln-iPrLys-Ala-Phe-Hse-PyrS2-Phe-BztA-Hse-NH2 | 1471 |
| I-1923 | Ac-PL3-Asn-Cha-B5-Asp-Gln-iPrLys-Ala-3PyrA-Asn-PyrS2-Phe-BztA-Hse-NH2 | 1472 |
| I-1924 | Ac-PL3-Asn-Cha-B5-Asp-Gln-iPrLys-Ala-3C1F-Asn-PyrS2-Phe-BztA-Hse-NH2 | 1473 |
| I-1925 | Ac-PL3-Asn-Cha-B5-Asp-Gln-iPrLys-Ala-3cbmf-Asn-PyrS2-Phe-BztA-Hse-NH2 | 1474 |
| I-1926 | Ac-PL3-Asn-Cha-B5-Asp-Gln-iPrLys-Ala-3OMeF-Asn-PyrS2-Phe-BztA-Hse-NH2 | 1475 |
| I-1927 | Ac-PL3-Asn-Cha-B5-Asp-Gln-iPrLys-Ala-Phe-Asn-PyrS2-3PyrA-BztA-Hse-NH2 | 1476 |
| I-1928 | Ac-PL3-Asn-Cha-B5-Asp-Gln-iPrLys-Ala-Phe-Asn-PyrS2-3C1F-BztA-Hse-NH2 | 1477 |
| I-1929 | Ac-PL3-Asn-Cha-B5-Asp-Gln-iPrLys-Ala-Phe-Asn-PyrS2-3cbmf-BztA-Hse-NH2 | 1478 |
| I-1930 | Ac-PL3-Asn-Cha-B5-Asp-Gln-iPrLys-Ala-Phe-Asn-PyrS2-3OMeF-BztA-Hse-NH2 | 1479 |
| I-1931 | Ac-PL3-Asn-Cha-B5-Asp-Gln-iPrLys-Ser-Phe-Asn-PyrS2-Phe-BztA-Hse-NH2 | 1480 |
| I-1932 | Ac-PL3-Hse-Cha-B5-Asp-Gln-iPrLys-Ala-Phe-Asn-PyrS2-Phe-BztA-Hse-NH2 | 1481 |
| I-1933 | Ac-PL3-Asn-Cha-B5-Asp-Hse-iPrLys-Ala-Phe-Asn-PyrS2-Phe-BztA-Hse-NH2 | 1482 |
| I-1934 | Ac-PL3-Asp-Npg-B5-Asp-TfeGA-Ala-Ala-Phe-[H4IAP]GlnR-PyrS2-3Thi-BztA-Gln- | 1483 |
| | NH2 | |
| I-1935 | Ac-PL3-Asp-Npg-B5-Asp-TfeGA-Ala-Ala-Phe-[TfePpz]GlnR-PyrS2-3Thi-BztA-Gln- | 1484 |
| | NH2 | |
| I-1936 | Ac-PL3-Asp-Npg-B5-Asp-TfeGA-Ala-Ala-Phe-[Me2NPrPip]GlnR-PyrS2-3Thi-BztA- | 1485 |
| | Gln-NH2 | |
| I-1937 | Ac-PL3-Asp-Npg-B5-Asp-TfeGA-Ala-Ala-Phe-[4MePpzPip]GlnR-PyrS2-3Thi-BztA- | 1486 |
| | Gln-NH2 | |
| I-1938 | Ac-PL3-Asp-Npg-B5-Asp-TfeGA-Ala-Ala-Phe-[4PyPip]GlnR-PyrS2-3Thi-BztA-Gln- | 1487 |
| | NH2 | |
| I-1939 | Ac-PL3-Asp-Npg-B5-Asp-TfeGA-Ala-Ala-Phe-[RDMAPyr]GlnR-PyrS2-3Thi-BztA- | 1488 |
| | Gln-NH2 | |
| I-1940 | Ac-PL3-Asp-Npg-B5-Asp-TfeGA-Ala-Ala-Phe-[4Pippip]GlnR-PyrS2-3Thi-BztA-Gln- | 1489 |
| | NH2 | |
| I-1941 | Ac-PL3-Asp-Npg-B5-Asp-TfeGA-Ala-Ala-Phe-[Ppz]GlnR-PyrS2-3Thi-BztA-Gln-NH2 | 1490 |
| I-1942 | Ac-PL3-Asp-Npg-B5-Asp-TfeGA-Ala-Ala-Phe-[NHBn]GlnR-PyrS2-3Thi-BztA-Gln- | 1491 |
| | NH2 | |
| I-1943 | Ac-PL3-Asp-Npg-B5-Asp-TfeGA-Ala-Ala-Phe-[Morph]GlnR-PyrS2-3Thi-BztA-Gln- | 1492 |
| | NH2 | |
| I-1944 | Ac-PL3-Asp-Npg-B5-AspSH-Asp-Ala-Ala-Phe-Leu-PyrS2-3Thi-BztA-Gln-NH2 | 1493 |
| I-1945 | Ac-PL3-Asp-Npg-B5-AspSH-Asp-Ala-Ala-Phe-Leu-PyrS2-3Thi-BztA-Gln-NH2 | 1493 |
| I-1946 | Ac-PL3-Asp-Npg-B5-Asp-AspSH-Ala-Ala-Phe-Leu-PyrS2-3Thi-BztA-Gln-NH2 | 1494 |
| I-1947 | Ac-PL3-Asp-Npg-B5-Asp-AspSH-Ala-Ala-Phe-Leu-PyrS2-3Thi-BztA-Gln-NH2 | 1494 |
| I-1948 | Ac-PL3-Asp-Npg-B5-Asp-GluSH-Ala-Ala-Phe-Leu-PyrS2-3Thi-BztA-Gln-NH2 | 1495 |
| I-1949 | Ac-PL3-Asp-Npg-B5-Asp-GluSH-Ala-Ala-Phe-Leu-PyrS2-3Thi-BztA-Gln-NH2 | 1495 |
| I-1950 | Ac-PL3-Asp-Npg-B5-[EtSSpy]AspE-Asp-Ala-Ala-Phe-Leu-PyrS2-3Thi-BztA-Gln-NH2 | 1496 |
| I-1951 | Ac-PL3-Asp-Npg-B5-Asp-[EtSSpy]GluE-Ala-Ala-Phe-Leu-PyrS2-3Thi-BztA-Gln-NH2 | 1497 |
| I-1952 | Ac-PL3-[EtSSPh]AspE-Npg-B5-Asp-Asp-Ala-Ala-Phe-Leu-PyrS2-3Thi-BztA-Gln-NH2 | 1498 |
| I-1953 | Ac-PL3-[EtSSPh]AspE-Npg-B5-Asp-Asp-Ala-Ala-Phe-Leu-PyrS2-3Thi-BztA-Gln-NH2 | 1498 |
| I-1954 | Ac-PL3-Asp-Npg-B5-[EtSSPh]AspE-Asp-Ala-Ala-Phe-Leu-PyrS2-3Thi-BztA-Gln-NH2 | 1499 |
| I-1955 | Ac-PL3-Asp-Npg-B5-[EtSSPh]AspE-Asp-Ala-Ala-Phe-Leu-PyrS2-3Thi-BztA-Gln-NH2 | 1499 |
| I-1956 | Ac-PL3-Asp-Npg-B5-Asp-[EtSSPh]AspE-Ala-Ala-Phe-Leu-PyrS2-3Thi-BztA-Gln-NH2 | 1500 |
| I-1957 | Ac-PL3-Asp-Npg-B5-Asp-[EtSSPh]GluE-Ala-Ala-Phe-Leu-PyrS2-3Thi-BztA-Gln-NH2 | 1501 |
| I-1958 | Ac-PL3-Asp-Npg-B5-Asp-[EtSSPh]GluE-Ala-Ala-Phe-Leu-PyrS2-3Thi-BztA-Gln-NH2 | 1501 |
| I-1959 | Ac-PL3-[EtSSHex]AspE-Npg-B5-Asp-Asp-Ala-Ala-Phe-Leu-PyrS2-3Thi-BztA-Gln- | 1502 |
| | NH2 | |
| I-1960 | Ac-PL3-[EtSSHex]AspE-Npg-B5-Asp-Asp-Ala-Ala-Phe-Leu-PyrS2-3Thi-BztA-Gln- | 1502 |
| | NH2 | |
| I-1961 | Ac-PL3-Asp-Npg-B5-[EtSSHex]AspE-Asp-Ala-Ala-Phe-Leu-PyrS2-3Thi-BztA-Gln- | 1503 |
| | NH2 | |
| I-1962 | Ac-PL3-Asp-Npg-B5-Asp-[EtSSHex]AspE-Ala-Ala-Phe-Leu-PyrS2-3Thi-BztA-Gln- | 1504 |
| | NH2 | |
| I-1963 | Ac-PL3-[EtSSEt]AspE-Npg-B5-Asp-Asp-Ala-Ala-Phe-Leu-PyrS2-3Thi-BztA-Gln-NH2 | 1505 |
| I-1964 | Ac-PL3-Asp-Npg-B5-[EtSSEt]AspE-Asp-Ala-Ala-Phe-Leu-PyrS2-3Thi-BztA-Gln-NH2 | 1506 |
| I-1965 | Ac-PL3-Asp-Npg-B5-Asp-[EtSSEt]AspE-Ala-Ala-Phe-Leu-PyrS2-3Thi-BztA-Gln-NH2 | 1507 |
| I-1966 | Ac-PL3-Asp-Npg-B5-Asp-[EtSSEt]GluE-Ala-Ala-Phe-Leu-PyrS2-3Thi-BztA-Gln-NH2 | 1508 |
| I-1967 | Ac-PL3-Asp-Npg-B5-Asp-R3COOPipA-Ala-Ala-Phe-Leu-PyrS2-3Thi-BztA-Gln-NH2 | 1509 |
| I-1968 | Ac-PL3-Asp-Npg-B5-Asp-S2COOPipA-Ala-Ala-Phe-Leu-PyrS2-3Thi-BztA-Gln-NH2 | 1510 |
| I-1969 | Ac-PL3-Asp-Npg-B5-Asp-R2COOPipA-Ala-Ala-Phe-Leu-PyrS2-3Thi-BztA-Gln-NH2 | 1511 |
| I-1970 | Ac-PL3-Asp-Npg-B4-Asp-Asp-Ala-Ala-Phe-Leu-PyrS1-3Thi-BztA-Gln-NH2 | 1512 |
| I-1971 | Ac-PL3-Asp-Npg-B4-Asp-Asp-Ala-Ala-Phe-Leu-PyrS2-3Thi-BztA-Gln-NH2 | 1513 |
| I-1972 | Ac-PL3-Asp-Npg-B4-Asp-Asp-Ala-Ala-Phe-Leu-PyrS2-3Thi-BztA-Gln-NH2 | 1513 |
| I-1973 | Ac-PL3-Asp-Npg-B4-Asp-Asp-Ala-Ala-Phe-Leu-PyrS3-3Thi-BztA-Gln-NH2 | 1514 |
| I-1974 | Ac-PL3-Asp-Npg-B4-Asp-Asp-Ala-Ala-Phe-Leu-PyrS1-3Thi-BztA-Gln-NH2 | 1512 |
| I-1975 | Ac-PL3-Asp-Npg-B4-Asp-Asp-Ala-Ala-Phe-Leu-PyrS2-3Thi-BztA-Gln-NH2 | 1513 |
| I-1976 | Ac-PL3-Asp-Npg-B4-Asp-Asp-Ala-Ala-Phe-Leu-PyrS3-3Thi-BztA-Gln-NH2 | 1514 |
| I-1977 | Ac-PL3-Asp-Npg-S6-Asp-Asp-Ala-Ala-Phe-Leu-Aib-3Thi-BztA-Gln-NH2 | 1515 |
| I-1978 | Ac-PL3-Asp-Npg-S6-Asp-Asp-Ala-Ala-Phe-Leu-Aib-3Thi-BztA-Gln-NH2 | 1515 |
| I-1979 | Ac-PL3-Asp-Npg-S4-Asp-Asp-Ala-Ala-Phe-Leu-Aib-3Thi-BztA-Gln-NH2 | 1516 |
| I-1980 | Ac-PL3-Asp-Npg-S3-Asp-Asp-Ala-Ala-Phe-Leu-Aib-3Thi-BztA-Gln-NH2 | 1517 |
| I-1981 | Ac-PL3-Asp-Npg-S6-Asp-Asp-Ala-Ala-Phe-Leu-Aib-3Thi-BztA-Gln-NH2 | 1515 |
| I-1982 | Ac-PL3-Asp-Npg-S4-Asp-Asp-Ala-Ala-Phe-Leu-Aib-3Thi-BztA-Gln-NH2 | 1516 |
| I-1983 | Ac-PL3-Asp-Npg-S3-Asp-Asp-Ala-Ala-Phe-Leu-Aib-3Thi-BztA-Gln-NH2 | 1517 |
| I-1984 | ProAm5-Asp-Npg-B5-Asp-3COOHF-Ala-Ala-Phe-Leu-PyrS2-2C1F-BztA-Gln-NH2 | 1518 |
| I-1985 | ProAm6-Asp-Npg-B5-Asp-3COOHF-Ala-Ala-Phe-Leu-PyrS2-2C1F-BztA-Gln-NH2 | 1519 |
| I-1986 | ProAm6-Asp-Npg-B5-Asp-TfeGA-Ala-Ala-Phe-Thr-PyrS2-2C1F-BztA-Gln-NH2 | 1520 |
| I-1987 | Ac-PL3-Asp-Npg-B5-Asp-Asp-Ala-Ala-Phe-Asn-PyrS2-3Thi-BztA-Gln-NH2 | 448 |
| I-1988 | Ac-Pro-Asp-Npg-R6-Asp-Asp-Ala-Ala-Phe-Leu-PyrS2-3Thi-BztA-Gln-NH2 | 1521 |
| I-1989 | Ac-Pro-Asp-Npg-R4-Asp-Asp-Ala-Ala-Phe-Leu-PyrS2-3Thi-BztA-Gln-NH2 | 1522 |
| I-1990 | Ac-Pro-Asp-Npg-R3-Asp-Asp-Ala-Ala-Phe-Leu-PyrS2-3Thi-BztA-Gln-NH2 | 1523 |
| I-1991 | Ac-Pro-Asp-Npg-R6-Asp-Asp-Ala-Ala-Phe-Leu-PyrS2-3Thi-BztA-Gln-NH2 | 1521 |
| I-1992 | Ac-Pro-Asp-Npg-R4-Asp-Asp-Ala-Ala-Phe-Leu-PyrS2-3Thi-BztA-Gln-NH2 | 1522 |
| I-1993 | Ac-Pro-Asp-Npg-R3-Asp-Asp-Ala-Ala-Phe-Leu-PyrS2-3Thi-BztA-Gln-NH2 | 1523 |
| I-1994 | Ac-PL3-Asp-Npg-B5-Asn-Thr-Ala-Ala-Phe-Asn-PyrS2-hPhe-BztA-Gln-NH2 | 1524 |
| I-1995 | Ac-PL3-Asp-Npg-B5-Asn-Thr-Ala-Ala-Phe-Asn-PyrS2-hPhe-BztA-Gln-NH2 | 1524 |
| I-1996 | Ac-PL3-Asp-Npg-B5-Asn-Thr-Ala-Ala-Phe-Asn-PyrS2-3F3MeF-BztA-Gln-NH2 | 1525 |
| I-1997 | Ac-PL3-Asp-Npg-B5-Asn-Thr-Ala-Ala-Phe-Asn-PyrS2-3F3MeF-BztA-Gln-NH2 | 1525 |
| I-1998 | Ac-PL3-Asp-Npg-B5-Asn-Thr-Ala-Ala-Phe-Asn-PyrS2-2C1F-BztA-Gln-NH2 | 1526 |
| I-1999 | Ac-PL3-Asp-Npg-B5-Asn-Thr-Ala-Ala-Phe-Asn-PyrS2-2C1F-BztA-Gln-NH2 | 1526 |
| I-2000 | Ac-PL3-Asp-Npg-B5-Asn-Thr-Ala-Ala-Phe-Asn-PyrS2-3C1F-BztA-Gln-NH2 | 1527 |
| I-2001 | Ac-PL3-Asp-Npg-B5-Asn-Thr-Ala-Ala-Phe-Asn-PyrS2-3C1F-BztA-Gln-NH2 | 1527 |
| I-2002 | Ac-PL3-Asp-Ala-B5-Asn-Thr-Ala-Ala-Phe-Asn-PyrS2-3Thi-BztA-Gln-NH2 | 1528 |
| I-2003 | Ac-PL3-Asp-Ala-B5-Asn-Thr-Ala-Ala-Phe-Asn-PyrS2-3Thi-BztA-Gln-NH2 | 1528 |
| I-2004 | Ac-PL3-Asp-Npg-B5-Asn-Thr-iPrLys-Ala-Phe-Asn-PyrS2-3Thi-BztA-Gln-NH2 | 1529 |
| I-2005 | Ac-PL3-Asp-Npg-B5-Asn-Thr-iPrLys-Ala-Phe-Asn-PyrS2-3Thi-BztA-Gln-NH2 | 1529 |
| I-2006 | Ac-PL3-Asp-Npg-B5-Asn-Thr-Ala-iPrLys-Phe-Asn-PyrS2-3Thi-BztA-Gln-NH2 | 1530 |
| I-2007 | Ac-PL3-Asp-Npg-B5-Asn-Thr-Ala-Ala-hPhe-Asn-PyrS2-3Thi-BztA-Gln-NH2 | 1531 |
| I-2008 | Ac-PL3-Asp-Npg-B5-Asn-Thr-Ala-Ala-hPhe-Asn-PyrS2-3Thi-BztA-Gln-NH2 | 1531 |
| I-2009 | Ac-PL3-Asp-Npg-B5-Asn-Thr-Ala-Ala-3F3MeF-Asn-PyrS2-3Thi-BztA-Gln-NH2 | 1532 |
| I-2010 | Ac-PL3-Asp-Npg-B5-Asn-Thr-Ala-Ala-3F3MeF-Asn-PyrS2-3Thi-BztA-Gln-NH2 | 1532 |
| I-2011 | Ac-PL3-Asp-Npg-B5-Asn-Thr-Ala-Ala-2C1F-Asn-PyrS2-3Thi-BztA-Gln-NH2 | 1533 |
| I-2012 | Ac-PL3-Asp-Npg-B5-Asn-Thr-Ala-Ala-2C1F-Asn-PyrS2-3Thi-BztA-Gln-NH2 | 1533 |
| I-2013 | Ac-PL3-Asp-Npg-B5-Asn-Thr-Ala-Ala-3C1F-Asn-PyrS2-3Thi-BztA-Gln-NH2 | 1534 |
| I-2014 | Ac-PL3-Asp-Npg-B5-Asn-Thr-Ala-Ala-3C1F-Asn-PyrS2-3Thi-BztA-Gln-NH2 | 1534 |
| I-2015 | Ac-PL3-Asp-Npg-B5-Asn-Thr-Ala-Ala-Tyr-Asn-PyrS2-3Thi-BztA-Gln-NH2 | 1535 |
| I-2016 | Ac-PL3-Asp-Npg-B5-Asn-Thr-Ala-Ala-Tyr-Asn-PyrS2-3Thi-BztA-Gln-NH2 | 1535 |
| I-2017 | Ac-PL3-Asp-Npg-B5-Asp-Asp-Ala-Ala-Phe-Asn-PyrR2-3Thi-BztA-Gln-NH2 | 1536 |
| I-2018 | Ac-PL3-Asp-Npg-B5-Asp-Asp-Ala-Ala-Phe-Asn-PyrR2-3Thi-BztA-Gln-NH2 | 1536 |
| I-2019 | Ac-PL3-Asp-Npg-B5-Asp-Asp-Ala-Ala-Phe-Leu-PyrR2-3Thi-BztA-Gln-NH2 | 1537 |
| I-2020 | Ac-PL3-Asp-Npg-B5-Asp-Asp-Ala-Ala-Phe-Leu-PyrR2-3Thi-BztA-Gln-NH2 | 1537 |
| I-2021 | Ac-PL3-Asp-Npg-B5-Asp-Asp-Ala-Ala-Phe-Asn-PyrR2-3Thi-BztA-Gln-NH2 | 1536 |
| I-2022 | Ac-PL3-Asp-Npg-B5-Asp-Asp-Ala-Ala-Phe-Leu-PyrR2-3Thi-BztA-Gln-NH2 | 1537 |
| I-2023 | Ac-PL3-Asp-Npg-B5-Asp-3COOHF-Ala-Ala-Phe-Ala-PyrS2-2C1F-BztA-Ala-NH2 | 1538 |
| I-2024 | Ac-PL3-Asp-Npg-B5-Asp-3COOHF-Ala-Ala-Phe-Ala-PyrS2-2C1F-BztA-Val-NH2 | 1539 |
| I-2025 | Ac-PL3-Asp-Npg-B5-Asp-3COOHF-Ala-Ala-Phe-Ala-PyrS2-2C1F-BztA-Phe-NH2 | 1540 |
| I-2026 | Ac-PL3-Asp-Npg-B5-Asp-3COOHF-Ala-Ala-Phe-Ala-PyrS2-2C1F-BztA-dAla-NH2 | 1541 |
| I-2027 | Ac-PL3-Asp-Npg-B5-Asp-3COOHF-Ala-Ala-Phe-Ser-PyrS2-2C1F-BztA-Ala-NH2 | 1542 |
| I-2028 | Ac-PL3-Asp-Npg-B5-Asp-3COOHF-Ala-Ala-Phe-Ser-PyrS2-2C1F-BztA-Val-NH2 | 1543 |
| I-2029 | Ac-PL3-Asp-Npg-B5-Asp-3COOHF-Ala-Ala-Phe-Ser-PyrS2-2C1F-BztA-Phe-NH2 | 1544 |
| I-2030 | Ac-PL3-Asp-Npg-B5-Asp-3COOHF-Ala-Ala-Phe-Ser-PyrS2-2C1F-BztA-dAla-NH2 | 1545 |
| I-2031 | Ac-PL3-Asp-Npg-B5-Asp-3COOHF-Ala-Ala-Phe-Asn-PyrS2-2C1F-BztA-Ala-NH2 | 1546 |
| I-2032 | Ac-PL3-Asp-Npg-B5-Asp-3COOHF-Ala-Ala-Phe-Asn-PyrS2-2C1F-BztA-Val-NH2 | 1547 |
| I-2033 | Ac-PL3-Asp-Npg-B5-Asp-3COOHF-Ala-Ala-Phe-Asn-PyrS2-2C1F-BztA-Phe-NH2 | 1548 |
| I-2034 | Ac-PL3-Asp-Npg-B5-Asp-3COOHF-Ala-Ala-Phe-Asn-PyrS2-2C1F-BztA-dAla-NH2 | 1549 |
| I-2035 | Ac-PL3-Asp-Npg-B5-Asp-3COOHF-Ala-Ala-Phe-Ala-PyrS2-2C1F-BztA-Asn-NH2 | 1550 |
| I-2036 | Ac-PL3-Asp-Npg-B5-Asp-3COOHF-Ala-Ala-Phe-Ala-PyrS2-2C1F-BztA-Asn-NH2 | 1550 |
| I-2037 | Ac-PL3-Asp-Npg-B5-Asp-3COOHF-Ala-Ala-Phe-Phe-PyrS2-2C1F-BztA-Asn-NH2 | 1551 |
| I-2038 | Ac-PL3-Asp-Npg-B5-Asp-3COOHF-Ala-Ala-Phe-Ser-PyrS2-2C1F-BztA-Asn-NH2 | 1552 |
| I-2039 | Ac-PL3-Asp-Npg-B5-Asp-3COOHF-Ala-Ala-Phe-Ser-PyrS2-2C1F-BztA-Asn-NH2 | 1552 |
| I-2040 | Ac-PL3-Asp-Npg-B5-Asp-3COOHF-Ala-Ala-Phe-Leu-PyrS2-2C1F-BztA-Asn-Ala-Ala- | 1553 |
| | Ala-NH2 | |
| I-2041 | Ac-PL3-Asp-Npg-B5-Asp-3COOHF-Ala-Ala-Phe-Leu-PyrS2-2C1F-BztA-Asn-Ala-Ala- | 1554 |
| | dAla-NH2 | |
| I-2042 | Ac-PL3-Asp-Npg-B5-Asp-3COOHF-Ala-Ala-Phe-Ala-PyrS2-3Thi-BztA-Asn-NH2 | 1555 |
| I-2043 | Ac-PL3-Asp-Npg-B5-Asp-3COOHF-Ala-Ala-Phe-Leu-PyrS2-3Thi-BztA-Asn-NH2 | 1556 |
| I-2044 | Ac-PL3-Asp-Npg-B5-Asp-3COOHF-Ala-Ala-Phe-Phe-PyrS2-3Thi-BztA-Asn-NH2 | 1557 |
| I-2045 | Ac-PL3-Asp-Npg-B5-Asp-3COOHF-Ala-Ala-Phe-Ser-PyrS2-3Thi-BztA-Asn-NH2 | 1558 |
| I-2046 | Ac-PL3-Asp-Npg-B5-Asp-3COOHF-Ala-Ala-Phe-Leu-PyrS2-3Thi-BztA-Asn-Ala-Ala- | 1559 |
| | Ala-NH2 | |
| I-2047 | Ac-PL3-Asp-Npg-B5-Asp-3COOHF-Ala-Ala-Phe-Leu-PyrS2-3Thi-BztA-Asn-Ala-Ala- | 1560 |
| | dAla-NH2 | |
| I-2048 | Ac-PL3-Asp-Npg-B5-Asp-TfeGA-Ala-Ala-3CH2NMe2F-Leu-PyrS2-3Thi-BztA-Gln- | 1561 |
| | NH2 | |
| I-2049 | Ac-PL3-Asp-Npg-B5-Asp-3COOHF-Asn-Ala-Phe-Leu-PyrS2-3Thi-BztA-Ala-NH2 | 1562 |
| I-2050 | Ac-PL3-Asp-Npg-B5-Asp-3COOHF-Ser-Ala-Phe-Leu-PyrS2-3Thi-BztA-Ala-NH2 | 1563 |
| I-2051 | Ac-PL3-Asp-Npg-B5-Asp-3COOHF-Thr-Ala-Phe-Leu-PyrS2-3Thi-BztA-Ala-NH2 | 1564 |
| I-2052 | Ac-PL3-Asp-Npg-B5-Asp-3COOHF-Gln-Ala-Phe-Phe-PyrS2-3Thi-BztA-Ala-NH2 | 1565 |
| I-2053 | Ac-PL3-Asp-Npg-B5-Asp-3COOHF-Gln-Ala-Phe-Ala-PyrS2-3Thi-BztA-Ala-NH2 | 1566 |
| I-2054 | Ac-PL3-Asp-Npg-B5-Asp-3COOHF-Gln-Ala-Phe-Val-PyrS2-3Thi-BztA-Ala-NH2 | 1567 |
| I-2055 | Ac-PL3-Asp-Npg-B5-Asp-3COOHF-Gln-Ala-Phe-Leu-PyrS2-3Thi-BztA-Val-NH2 | 1568 |
| I-2056 | Ac-PL3-Asp-Npg-B5-Asp-3COOHF-Gln-Ala-Phe-Leu-PyrS2-3Thi-BztA-Leu-NH2 | 1569 |
| I-2057 | Ac-PL3-Asp-Npg-B5-Asp-3COOHF-Gln-Ala-Phe-Leu-PyrS2-2F3MeF-BztA-Ala-NH2 | 1570 |
| I-2058 | Ac-PL3-Asp-Npg-B5-Asp-3COOHF-Asn-Ala-Phe-Leu-PyrS2-2F3MeF-BztA-Ala-NH2 | 1571 |
| I-2059 | Ac-PL3-Asp-Npg-B5-Asp-3COOHF-Ser-Ala-Phe-Leu-PyrS2-2F3MeF-BztA-Ala-NH2 | 1572 |
| I-2060 | Ac-PL3-Asp-Npg-B5-Asp-3COOHF-Thr-Ala-Phe-Leu-PyrS2-2F3MeF-BztA-Ala-NH2 | 1573 |
| I-2061 | Ac-PL3-Asp-Npg-B5-Asp-3COOHF-Ala-Ala-Phe-Leu-PyrS2-3Thi-BztA-Met2O-NH2 | 1574 |
| I-2062 | Ac-PL3-Asp-Npg-B5-Asp-3COOHF-Ala-Ala-Phe-Leu-PyrS2-2C1F-BztA-Met2O-NH2 | 1575 |
| I-2063 | Ac-PL3-Asp-Npg-B5-Asp-3COOHF-Ala-Ala-Phe-Leu-PyrS2-2F3MeF-BztA-Met2O- | 1576 |
| | NH2 | |
| I-2064 | Ac-PL3-Asp-Npg-B5-Asp-3COOHF-Met2O-Ala-Phe-Leu-PyrS2-3Thi-BztA-Ala-NH2 | 1577 |
| I-2065 | Ac-PL3-Asn-Cha-B5-Asp-Gln-iPrLys-Ala-Phe-Asn-PyrS2-Phe-BztA-Ala-NH2 | 1578 |
| I-2066 | Ac-PL3-Asn-Cha-B5-Asp-Gln-His-Ala-Phe-Asn-PyrS2-Phe-BztA-Hse-NH2 | 1579 |
| I-2067 | Ac-PL3-Asn-Cha-B5-Asp-Gln-Gln-Ala-Phe-Asn-PyrS2-Phe-BztA-Hse-NH2 | 1580 |
| I-2068 | Ac-PL3-Asn-Cha-B5-Asp-Gln-iPrLys-Ala-Phe-Asn-PyrS2-Phe-BztA-Phe-NH2 | 1581 |
| I-2069 | Ac-PL3-Asn-Cha-B5-Asp-Gln-His-Ala-Phe-Asn-PyrS2-Phe-BztA-Ala-NH2 | 1582 |
| I-2070 | Ac-PL3-Asn-Cha-B5-Asp-Gln-Gln-Ala-Phe-Asn-PyrS2-Phe-BztA-Ala-NH2 | 1583 |
| I-2071 | Ac-PL3-Asn-Cha-B5-Asp-Gln-iPrLys-Ala-Phe-Asn-PyrS2-4Thz-BztA-Hse-NH2 | 1584 |
| I-2072 | Ac-PL3-Asn-Cha-B5-Asp-Gln-iPrLys-Ala-4Thz-Asn-PyrS2-Phe-BztA-Hse-NH2 | 1585 |
| I-2073 | Ac-PL3-Asn-Cha-B5-Asp-Gln-iPrLys-Ala-Phe-Asn-PyrS2-Phe-BztA-NH2 | 1586 |
| I-2074 | Ac-PL3-Asn-Cha-B5-Asp-Gln-iPrLys-Ala-Phe-Asn-PyrS2-3Thi-BztA-Hse-NH2 | 1587 |
| I-2075 | Ac-PL3-Asn-Cha-B5-Asp-Gln-iPrLys-Ala-Phe-Asn-PyrS2-2Thi-BztA-Hse-NH2 | 1588 |
| I-2076 | Ac-PL3-Asp-Npg-B5-Asp-[CH2CH2CO2H]TriAzDab-Ala-Ala-Phe-Leu-PyrS2-3Thi- | 1589 |
| | BztA-Gln-NH2 | |
| I-2077 | Ac-PL3-Asp-Npg-B5-Asp-[CH2CMe2CO2H]TriAzDab-Ala-Ala-Phe-Leu-PyrS2-3Thi- | 1590 |
| | BztA-Gln-NH2 | |
| I-2078 | Ac-PL3-Asp-Npg-B5-Asp-[CH2CO2H]TriAzDab-Ala-Ala-Phe-Leu-PyrS2-3Thi-BztA- | 1591 |
| | Gln-NH2 | |
| I-2079 | Ac-PL3-Asp-Npg-B5-Asp-[CMe2CO2H]TriAzDab-Ala-Ala-Phe-Leu-PyrS2-3Thi- | 1592 |
| | BztA-Gln-NH2 | |
| I-2080 | Ac-PL3-Asp-Npg-B5-Asp-[2COOHPh]TriAzDab-Ala-Ala-Phe-Leu-PyrS2-3Thi-BztA- | 1593 |
| | Gln-NH2 | |
| I-2081 | Ac-PL3-Asp-Npg-B5-Asp-[2COOHPh]TriAzDab-Ala-Ala-Phe-Leu-PyrS2-3Thi-BztA- | 1593 |
| | Gln-NH2 | |
| I-2082 | Ac-PL3-Asp-Npg-B5-Asp-[CH2CH2CO2H]TriAzDap-Ala-Ala-Phe-Leu-PyrS2-3Thi- | 1594 |
| | BztA-Gln-NH2 | |
| I-2083 | Ac-PL3-Asp-Npg-B5-Asp-[CH2CMe2CO2H]TriAzDap-Ala-Ala-Phe-Leu-PyrS2-3Thi- | 1595 |
| | BztA-Gln-NH2 | |
| I-2084 | Ac-PL3-Asp-Npg-B5-Asp-[CH2CO2H]TriAzDap-Ala-Ala-Phe-Leu-PyrS2-3Thi-BztA- | 1596 |
| | Gln-NH2 | |
| I-2085 | Ac-PL3-Asp-Npg-B5-Asp-[CMe2CO2H]TriAzDap-Ala-Ala-Phe-Leu-PyrS2-3Thi- | 1597 |
| | BztA-Gln-NH2 | |
| I-2086 | Ac-PL3-Asp-Npg-B5-Asp-[2COOHPh]TriAzDap-Ala-Ala-Phe-Leu-PyrS2-3Thi-BztA- | 1598 |
| | Gln-NH2 | |
| I-2087 | Ac-PL3-Asp-Npg-B5-Asp-[2COOH4NH2Ph]Dap-Ala-Ala-Phe-Leu-PyrS2-3Thi-BztA- | 1599 |
| | Gln-NH2 | |
| I-2088 | Ac-PL3-Asp-Npg-B5-Asp-3COOHF-Ala-Ala-Phe-Leu-PyrS2-3Thi-BztA-dGln-NH2 | 1600 |
| I-2089 | Ac-PL3-Asp-Npg-B5-Asp-3COOHF-Ala-Ala-Phe-Leu-PyrS2-2F3MeF-BztA-dGln-NH2 | 1601 |
| I-2090 | Ac-PL3-Asp-Npg-B5-Asp-3COOHF-Ala-Ala-Phe-Leu-PyrS2-3Thi-BztA-dAla-NH2 | 1602 |
| I-2091 | Ac-PL3-Asp-Npg-B5-Asp-3COOHF-Ala-Ala-Phe-Leu-PyrS2-2F3MeF-BztA-dAla-NH2 | 1603 |
| I-2092 | Ac-PL3-Asp-Npg-B5-Asp-3COOHF-Ala-Ala-Phe-Phe-PyrS2-3Thi-BztA-dGln-NH2 | 1604 |
| I-2093 | Ac-PL3-Asp-Npg-B5-Asp-3COOHF-Ala-Ala-Phe-Phe-PyrS2-2F3MeF-BztA-dGln-NH2 | 1605 |
| I-2094 | Ac-PL3-Asp-Npg-B5-Asp-3COOHF-Ala-Ala-Phe-Phe-PyrS2-3Thi-BztA-dAla-NH2 | 1606 |
| I-2095 | Ac-PL3-Asp-Npg-B5-Asp-3COOHF-Ala-Ala-Phe-Phe-PyrS2-2F3MeF-BztA-dAla-NH2 | 1607 |
| I-2096 | Ac-PL3-Asp-Npg-B5-Asp-3COOHF-Ala-dAla-Phe-Leu-PyrS2-3Thi-BztA-Ala-NH2 | 1608 |
| I-2097 | Ac-PL3-Asp-Npg-B5-Asp-3COOHF-Ala-dAla-Phe-Leu-PyrS2-3Thi-BztA-Ala-NH2 | 1608 |
| I-2098 | Ac-PL3-Asp-Npg-B5-Asp-3COOHF-Ala-dAla-Phe-Leu-PyrS2-2F3MeF-BztA-Ala-NH2 | 1609 |
| I-2099 | Ac-PL3-Asp-Npg-B5-Asp-3COOHF-Ala-dAla-Phe-Leu-PyrS2-2F3MeF-BztA-Ala-NH2 | 1609 |
| I-2100 | Ac-PL3-Asp-Npg-B5-Asp-3COOHF-Ala-dAla-Phe-Leu-PyrS2-3Thi-BztA-dAla-NH2 | 1610 |
| I-2101 | Ac-PL3-Asp-Npg-B5-Asp-3COOHF-Ala-dAla-Phe-Leu-PyrS2-2F3MeF-BztA-dAla- | 1611 |
| | NH2 | |
| I-2102 | Ac-PL3-Asn-Cha-B5-Asp-Gln-iPrLys-Ala-Phe-iPrLys-PyrS2-Phe-BztA-Hse-NH2 | 1612 |
| I-2103 | Ac-PL3-Ser-Cha-B5-Asp-Gln-iPrLys-Ala-Phe-Asn-PyrS2-Phe-BztA-Hse-NH2 | 1613 |
| I-2104 | Ac-PL3-Thr-Cha-B5-Asp-Gln-iPrLys-Ala-Phe-Asn-PyrS2-Phe-BztA-Hse-NH2 | 1614 |
| I-2105 | Ac-PL3-Gln-Cha-B5-Asp-Gln-iPrLys-Ala-Phe-Asn-PyrS2-Phe-BztA-Hse-NH2 | 1615 |
| I-2106 | Ac-PL3-His-Cha-B5-Asp-Gln-iPrLys-Ala-Phe-Asn-PyrS2-Phe-BztA-Hse-NH2 | 1616 |
| I-2107 | Ac-PL3-4Thz-Cha-B5-Asp-Gln-iPrLys-Ala-Phe-Asn-PyrS2-Phe-BztA-Hse-NH2 | 1617 |
| I-2108 | Ac-PL3-4Tria-Cha-B5-Asp-Gln-iPrLys-Ala-Phe-Asn-PyrS2-Phe-BztA-Hse-NH2 | 1618 |
| I-2109 | Ac-PL3-Asn-Cha-B5-Asp-Gln-iPrLys-Ala-Phe-Asn-PyrS2-Phe-BztA-His-NH2 | 1619 |
| I-2110 | Ac-PL3-Asn-Cha-B5-Asp-Gln-iPrLys-Ala-Phe-Asn-PyrS2-Phe-BztA-Lys-NH2 | 1620 |
| I-2111 | Ac-PL3-Asn-Cha-B5-Asp-Gln-iPrLys-Ala-Phe-Asn-PyrS2-Phe-BztA-Asn-NH2 | 1621 |
| I-2112 | Ac-PL3-Asn-Cha-B5-Asp-Gln-iPrLys-Ala-Phe-Asn-PyrS2-Phe-BztA-Thr-NH2 | 1622 |
| I-2113 | Ac-PL3-Asn-Cha-B5-Asp-Gln-iPrLys-Ala-Phe-Asn-PyrS2-Phe-BztA-Gln-NH2 | 1623 |
| I-2114 | Ac-PL3-Asp-Npg-B5-Asp-hTyr-Ala-Ala-3COOHF-Asn-PyrS2-3Thi-BztA-Gln-NH2 | 1624 |
| I-2115 | Ac-PL3-Asp-Npg-B5-Asp-hTyr-Ala-Ala-3COOHF-Asn-PyrS2-3Thi-BztA-Gln-NH2 | 1624 |
| I-2116 | Ac-PL3-Asp-Npg-B5-Asp-hTyr-Ala-Ala-3COOHF-Hse-PyrS2-3Thi-BztA-Gln-NH2 | 1625 |
| I-2117 | Ac-PL3-Asp-Npg-B5-Asp-hTyr-Ala-Ala-3COOHF-Hse-PyrS2-3Thi-BztA-Gln-NH2 | 1625 |
| I-2118 | Ac-PL3-Asp-Npg-B5-Asp-His-Ala-Ala-3COOHF-Asn-PyrS2-3Thi-BztA-Gln-NH2 | 1626 |
| I-2119 | Ac-PL3-Asp-Npg-B5-Asp-His-Ala-Ala-3COOHF-Asn-PyrS2-3Thi-BztA-Gln-NH2 | 1626 |
| I-2120 | Ac-PL3-Asp-Npg-B5-Asp-His-Ala-Ala-3COOHF-Hse-PyrS2-3Thi-BztA-Gln-NH2 | 1627 |
| I-2121 | Ac-PL3-Asp-Npg-B5-Asp-His-Ala-Ala-3COOHF-Hse-PyrS2-3Thi-BztA-Gln-NH2 | 1627 |
| I-2122 | Ac-PL3-Asp-Npg-B5-Asp-His-Ala-Ala-3COOHF-Leu-PyrS2-3Thi-BztA-Gln-NH2 | 1628 |
| I-2123 | Ac-PL3-Asp-Npg-B5-Asp-His-Ala-Ala-3COOHF-Leu-PyrS2-3Thi-BztA-Gln-NH2 | 1628 |
| I-2124 | Ac-PL3-Asp-Npg-B5-Asp-Tyr-Ala-Ala-3COOHF-Hse-PyrS2-3Thi-BztA-Gln-NH2 | 1629 |
| I-2125 | Ac-PL3-Asp-Npg-B5-Asp-Tyr-Ala-Ala-3COOHF-Hse-PyrS2-3Thi-BztA-Gln-NH2 | 1629 |
| I-2126 | Ac-PL3-Asp-Npg-B5-Asp-Tyr-Ala-Ala-3COOHF-Leu-PyrS2-3Thi-BztA-Gln-NH2 | 1630 |
| I-2127 | Ac-PL3-Asp-Npg-B5-Asp-Trp-Ala-Ala-3COOHF-Asn-PyrS2-3Thi-BztA-Gln-NH2 | 1631 |
| I-2128 | Ac-PL3-Asp-Npg-B5-Asp-Trp-Ala-Ala-3COOHF-Hse-PyrS2-3Thi-BztA-Gln-NH2 | 1632 |
| I-2129 | Ac-PL3-Asp-Npg-B5-Asp-Trp-Ala-Ala-3COOHF-Leu-PyrS2-3Thi-BztA-Gln-NH2 | 1633 |
| I-2130 | Ac-PL3-TfeGA-Npg-B5-Asn-Thr-Ala-Ala-Phe-Asn-PyrS2-3Thi-BztA-Gln-NH2 | 1634 |
| I-2131 | Ac-PL3-TfeGA-Npg-B5-Asn-Thr-Ala-Ala-Phe-Asn-PyrS2-3Thi-BztA-Gln-NH2 | 1634 |
| I-2132 | Ac-PL3-Aad-Npg-B5-Asn-Thr-Ala-Ala-Phe-Asn-PyrS2-3Thi-BztA-Gln-NH2 | 1635 |
| I-2133 | Ac-PL3-Aad-Npg-B5-Asn-Thr-Ala-Ala-Phe-Asn-PyrS2-3Thi-BztA-Gln-NH2 | 1635 |
| I-2134 | Ac-PL3-3COOHF-Npg-B5-Asn-Thr-Ala-Ala-Phe-Asn-PyrS2-3Thi-BztA-Gln-NH2 | 1636 |
| I-2135 | Ac-PL3-3COOHF-Npg-B5-Asn-Thr-Ala-Ala-Phe-Asn-PyrS2-3Thi-BztA-Gln-NH2 | 1636 |
| I-2136 | Ac-PL3-2COOHF-Npg-B5-Asn-Thr-Ala-Ala-Phe-Asn-PyrS2-3Thi-BztA-Gln-NH2 | 1637 |
| I-2137 | Ac-PL3-2COOHF-Npg-B5-Asn-Thr-Ala-Ala-Phe-Asn-PyrS2-3Thi-BztA-Gln-NH2 | 1637 |
| I-2138 | Ac-PL3-Asp-Npg-B5-Asn-Thr-Ala-Ala-Phe-Asn-PyrS2-3Thi-BztA-Trp-NH2 | 1638 |
| I-2139 | Ac-PL3-Asp-Npg-B5-Asn-Thr-Ala-Ala-Phe-Asn-PyrS2-3Thi-BztA-Trp-NH2 | 1638 |
| I-2140 | Ac-PL3-Asp-Npg-B5-Asn-Thr-Ala-Ala-Phe-Asn-PyrS2-3Thi-BztA-His-NH2 | 1639 |
| I-2141 | Ac-PL3-Asp-Npg-B5-Asn-Thr-Ala-Ala-Phe-Asn-PyrS2-3Thi-BztA-His-NH2 | 1639 |
| I-2142 | Ac-PL3-Asp-Npg-B5-Asn-Thr-Ala-Ala-Phe-Asn-PyrS2-3Thi-BztA-Ala-NH2 | 1640 |
| I-2143 | Ac-PL3-Asp-Npg-B5-Asn-Thr-Ala-Ala-Phe-Asn-PyrS2-3Thi-BztA-Ala-NH2 | 1640 |
| I-2144 | Ac-PL3-Asp-Npg-B5-Asn-Thr-Ala-Ala-Phe-Thr-PyrS2-3Thi-BztA-Phe-NH2 | 1641 |
| I-2145 | Ac-PL3-Asp-Npg-B5-Asn-Thr-Ala-Ala-Phe-Thr-PyrS2-3Thi-BztA-Phe-NH2 | 1641 |
| I-2146 | Ac-PL3-Asp-Npg-B5-Asn-Thr-Ala-Ala-Phe-His-PyrS2-3Thi-BztA-Gln-NH2 | 1642 |
| I-2147 | Ac-PL3-Asp-Npg-B5-Asn-Thr-Ala-Ala-Phe-His-PyrS2-3Thi-BztA-Gln-NH2 | 1642 |
| I-2148 | Ac-PL3-Asp-Npg-B5-Thr-Thr-Ala-Ala-Phe-Asn-PyrS2-3Thi-BztA-Gln-NH2 | 1643 |
| I-2149 | Ac-PL3-Asp-Npg-B5-Thr-Thr-Ala-Ala-Phe-Asn-PyrS2-3Thi-BztA-Gln-NH2 | 1643 |
| I-2150 | Ac-PL3-Asp-Npg-B5-Asp-TfeGA-Ala-Ala-3CO2PhF-Leu-PyrS2-3Thi-BztA-Gln-NH2 | 1644 |
| I-2151 | Ac-PL3-Asp-Npg-B5-Asp-TfeGA-Ala-Ala-3CO2PhF-Leu-PyrS2-3Thi-BztA-Gln-NH2 | 1644 |
| I-2152 | Ac-PL3-Asp-Npg-B5-Asp-3COOHF-Ala-Ala-Phe-Leu-PyrS2-3BrF-3Thi-Gln-NH2 | 1645 |
| I-2153 | Ac-PL3-Asp-Npg-B5-Asp-3COOHF-Ala-Ala-Phe-Leu-PyrS2-3BrF-Phe-Gln-NH2 | 1646 |
| I-2154 | Ac-PL3-Asp-Npg-B5-Asp-3COOHF-Ala-Ala-Phe-Leu-PyrS2-3BrF-Cba-Gln-NH2 | 1647 |
| I-2155 | Ac-PL3-Asp-Npg-B5-Asp-3COOHF-Ala-Ala-Phe-Leu-PyrS2-3BrF-2F3MeF-Gln-NH2 | 1648 |
| I-2156 | Ac-PL3-Asp-Npg-B3-Asp-Asp-Ala-Ala-Phe-Leu-PyrS2-3Thi-BztA-Gln-NH2 | 1649 |
| I-2157 | Ac-PL3-Asp-Npg-B3-Asp-Asp-Ala-Ala-Phe-Leu-PyrS3-3Thi-BztA-Gln-NH2 | 1650 |
| I-2158 | Ac-PL3-Asp-Npg-B3-Asp-Asp-Ala-Ala-Phe-Leu-PyrS4-3Thi-BztA-Gln-NH2 | 1651 |
| I-2159 | Ac-PL3-Asp-Npg-B3-Asp-Asp-Ala-Ala-Phe-Leu-PyrS4-3Thi-BztA-Gln-NH2 | 1651 |
| I-2160 | Ac-PL3-Asp-Npg-B3-Asp-Asp-Ala-Ala-Phe-Leu-PyrS2-3Thi-BztA-Gln-NH2 | 1649 |
| I-2161 | Ac-PL3-Asp-Npg-B3-Asp-Asp-Ala-Ala-Phe-Leu-PyrS3-3Thi-BztA-Gln-NH2 | 1650 |
| I-2162 | Ac-PL3-Asp-Npg-B3-Asp-Asp-Ala-Ala-Phe-Leu-PyrS4-3Thi-BztA-Gln-NH2 | 1651 |
| I-2163 | Ac-PL3-Asn-Cha-B5-Asp-Gln-iPrLys-Ala-Phe-Leu-PyrS2-3cbmf-BztA-Gln-NH2 | 1652 |
| I-2164 | Ac-PL3-Asn-Cha-B5-Asp-Gln-iPrLys-Ala-Phe-Asn-PyrS2-3cbmf-BztA-Gln-NH2 | 1653 |
| I-2165 | Ac-PL3-Asn-Cha-B5-Asp-Gln-iPrLys-Ala-Phe-Leu-PyrS2-Phe-BztA-Hse-NH2 | 1654 |
| I-2166 | Ac-PL3-Asn-Cha-B5-Asp-Gln-iPrLys-Ala-Phe-Leu-PyrS2-3cbmf-BztA-Hse-NH2 | 1655 |
| I-2167 | Ac-PL3-Asn-Cha-B5-Asp-Gln-iPrLys-Ala-Phe-Asn-PyrS2-Phe-BztA-Hse-NH2 | 1656 |
| I-2168 | Ac-PL3-Asn-Cha-B5-Asp-Gln-iPrLys-Ala-Phe-Asn-PyrS2-2cbmf-BztA-Hse-NH2 | 1657 |
| I-2169 | Ac-PL3-Asn-Cha-B5-Asp-Gln-iPrLys-Ala-Phe-Asn-PyrS2-4cbmf-BztA-Hse-NH2 | 1658 |
| I-2170 | Ac-PL3-Asn-Cha-B5-Asp-Gln-iPrLys-Ala-Phe-Asn-PyrS2-Asn-BztA-Hse-NH2 | 1659 |
| I-2171 | Ac-PL3-Asn-Cha-B5-Asp-Gln-iPrLys-Ala-Phe-Asn-PyrS2-Gln-BztA-Hse-NH2 | 1660 |
| I-2172 | Ac-PL3-Asn-Cha-B5-Asp-Gln-iPrLys-Ala-Phe-Asn-PyrS2-Tyr-BztA-Hse-NH2 | 1661 |
| I-2173 | Ac-PL3-Asn-Cha-B5-Asp-Gln-iPrLys-Ala-2cbmf-Asn-PyrS2-Phe-BztA-Hse-NH2 | 1662 |
| I-2174 | Ac-PL3-Asn-Cha-B5-Asp-Gln-iPrLys-Ala-4cbmf-Asn-PyrS2-Phe-BztA-Hse-NH2 | 1663 |
| I-2175 | Ac-PL3-Asp-Npg-B5-Asp-Asp-Ala-Ala-Phe-Asn-PyrS2-3Thi-Trp-Gln-NH2 | 1664 |
| I-2176 | Ac-PL3-Asp-Npg-B5-Asp-Asp-Ala-Ala-Phe-Asn-PyrS2-3Thi-4C1W-Gln-NH2 | 1665 |
| I-2177 | Ac-PL3-Asp-Npg-B5-Asp-Asp-Ala-Ala-Phe-Leu-PyrS2-3Thi-4C1W-Gln-NH2 | 1666 |
| I-2178 | Ac-PL3-Asp-Npg-B5-Asp-Asp-Ala-Ala-Phe-Asn-PyrS2-3Thi-4FW-Gln-NH2 | 1667 |
| I-2179 | Ac-PL3-Asp-Npg-B5-Asp-Asp-Ala-Ala-Phe-Leu-PyrS2-3Thi-4FW-Gln-NH2 | 1668 |
| I-2180 | Ac-PL3-Asp-Npg-B5-Asp-Asp-Ala-Ala-Phe-Asn-PyrS2-3Thi-7AzaW-Gln-NH2 | 1669 |
| I-2181 | Ac-PL3-Asp-Npg-B5-Asp-Asp-Ala-Ala-Phe-Leu-PyrS2-3Thi-7AzaW-Gln-NH2 | 1670 |
| I-2182 | Ac-PL3-Asp-Npg-B5-Asp-Asp-Ala-Ala-Phe-Asn-PyrS2-3Thi-34C1F-Gln-NH2 | 1671 |
| I-2183 | Ac-PL3-Asp-Npg-B5-Asp-Asp-Ala-Ala-Phe-Leu-PyrS2-3Thi-34C1F-Gln-NH2 | 1672 |
| I-2184 | Ac-PL3-Asp-Npg-B5-Asp-TfeGA-Ala-Ala-3SO2F-Leu-PyrS2-3Thi-BztA-Gln-NH2 | 1673 |
| I-2185 | Ac-PL3-Asp-Npg-B5-Asp-TfeGA-Ala-Ala-3SO2F-Leu-PyrS2-3Thi-BztA-Gln-NH2 | 1673 |
| I-2186 | Ac-PL3-Asp-Npg-B5-Asp-TfeGA-Ala-Ala-Phe-Leu-PyrS2-3Thi-BztA-BnBoroleK-NH2 | 1674 |
| I-2187 | Ac-PL3-Asp-Npg-B5-Asp-TfeGA-Ala-Ala-Phe-Leu-PyrS2-3Thi-BztA-BnBoroleK-NH2 | 1674 |
| I-2188 | Ac-PL3-Asp-Npg-B5-Asp-3COOHF-Me2Asn-Ala-Phe-Leu-PyrS2-3Thi-BztA-Ala-NH2 | 1675 |
| I-2189 | Ac-PL3-Asp-Npg-B5-Asp-3COOHF-nLeu-Ala-Phe-Leu-PyrS2-3Thi-BztA-Val-NH2 | 1676 |
| I-2190 | Ac-PL3-Asp-Npg-B5-Asp-3COOHF-Ser-Ala-Phe-Leu-PyrS2-3Thi-BztA-Val-NH2 | 1677 |
| I-2191 | Ac-PL3-Asp-Npg-B5-Asp-3COOHF-Me2Asn-Ala-Phe-Leu-PyrS2-3Thi-BztA-Val-NH2 | 1678 |
| I-2192 | Ac-PL3-Asp-Npg-B5-Asp-3COOHF-Gln-Ala-Phe-Leu-PyrS2-3Thi-BztA-Npg-NH2 | 1679 |
| I-2193 | Ac-PL3-Asp-Npg-B5-Asp-3COOHF-nLeu-Ala-Phe-Leu-PyrS2-3Thi-BztA-Npg-NH2 | 1680 |
| I-2194 | Ac-PL3-Asp-Npg-B5-Asp-3COOHF-Aib-Ala-Phe-Leu-PyrS2-3Thi-BztA-Npg-NH2 | 1681 |
| I-2195 | Ac-PL3-Asp-Npg-B5-Asp-3COOHF-Ser-Ala-Phe-Leu-PyrS2-3Thi-BztA-Npg-NH2 | 1682 |
| I-2196 | Ac-PL3-Asp-Npg-B5-Asp-3COOHF-Me2Asn-Ala-Phe-Leu-PyrS2-3Thi-BztA-Npg- | 1683 |
| | NH2 | |
| I-2197 | Ac-PL3-Asp-Npg-B5-Asn-Thr-Ala-Ala-Phe-Asn-PyrS1-3Thi-BztA-Gln-NH2 | 1684 |
| I-2198 | Ac-PL3-Asp-Npg-B5-Asn-Thr-Ala-Ala-Phe-Asn-PyrS1-3Thi-BztA-Gln-NH2 | 1684 |
| I-2199 | Ac-PL3-Asp-Npg-B5-Asn-Thr-Ala-Ala-Phe-Asn-PyrS3-3Thi-BztA-Gln-NH2 | 1685 |
| I-2200 | Ac-PL3-Asp-Npg-B5-Asn-Thr-Ala-Ala-Phe-Asn-S7-3Thi-BztA-Gln-NH2 | 1686 |
| I-2201 | Ac-PL3-Asp-Npg-B5-Asn-Thr-Ala-Ala-Phe-Asn-Az-3Thi-BztA-Gln-NH2 | 1687 |
| I-2202 | Ac-PL3-Asp-Npg-B5-Asp-4F3COOHF-Ala-Ala-Phe-Leu-PyrS2-3Thi-BztA-Ser-NH2 | 1688 |
| I-2203 | Ac-PL3-Asp-Npg-B5-Asp-4F3COOHF-Ala-Ala-Phe-Leu-PyrS2-2C1F-BztA-Gln-NH2 | 1689 |
| I-2204 | Ac-PL3-Asp-Npg-B5-Asp-4F3COOHF-Ala-Ala-Phe-Leu-PyrS2-2C1F-BztA-Gln-NH2 | 1689 |
| I-2205 | Ac-PL3-Asp-Npg-B5-Asp-4F3COOHF-Ala-Ala-Phe-Leu-PyrS2-2C1F-BztA-Ser-NH2 | 1690 |
| I-2206 | Ac-PL3-Asp-Npg-B5-Asp-4F3COOHF-Gln-Ala-Phe-Leu-PyrS2-3Thi-BztA-Ala-NH2 | 1691 |
| I-2207 | Ac-PL3-Asp-Npg-B5-Asp-4F3COOHF-Aib-Ala-Phe-Leu-PyrS2-3Thi-BztA-Ala-NH2 | 1692 |
| I-2208 | Ac-PL3-Asp-Npg-B5-Asp-5F3Me3COOHF-Ala-Ala-Phe-Leu-PyrS2-3Thi-BztA-Gln- | 1693 |
| | NH2 | |
| I-2209 | Ac-PL3-Asp-Npg-B5-Asp-5F3Me3COOHF-Ala-Ala-Phe-Leu-PyrS2-3Thi-BztA-Ser- | 1694 |
| | NH2 | |
| I-2210 | Ac-PL3-Asp-Npg-B5-Asp-5F3Me3COOHF-Ala-Ala-Phe-Leu-PyrS2-2C1F-BztA-Ser- | 1695 |
| | NH2 | |
| I-2211 | Ac-PL3-Asp-Npg-B5-Asp-5F3Me3COOHF-Gln-Ala-Phe-Leu-PyrS2-3Thi-BztA-Ala- | 1696 |
| | NH2 | |
| I-2212 | Ac-PL3-Asp-Npg-B5-Asp-5F3Me3COOHF-Aib-Ala-Phe-Leu-PyrS2-3Thi-BztA-Ala- | 1697 |
| | NH2 | |
| I-2213 | Ac-PL3-Asp-Npg-B5-Asn-Thr-Ala-Ala-Phe-Asn-SdN-3Thi-BztA-Gln-NH2 | 1698 |
| I-2214 | Ac-PL3-Asp-Npg-B5-Asp-3COOHF-Ala-Ala-Phe-Leu-PyrS2-[Ph]-3SF-Phe-Gln-NH2 | 1699 |
| I-2215 | Ac-PL3-Asp-Npg-B5-Asp-3COOHF-Ala-Ala-Phe-Leu-PyrS2-[CyPr]-3SF-Phe-Gln- | 1700 |
| | NH2 | |
| I-2216 | Ac-PL3-Asp-Npg-B5-Asp-3COOHF-Ala-Ala-Phe-Leu-PyrS2-3Thi-34C1F-Gln-NH2 | 1701 |
| I-2217 | Ac-PL3-Asp-Npg-B5-Asp-3COOHF-Gln-Ala-Phe-Val-PyrS2-3Thi-34C1F-Ala-NH2 | 1702 |
| I-2218 | Ac-PL3-Asp-Npg-B5-Asp-3COOHF-Ala-Ala-Phe-Leu-PyrS2-2F3MeF-34C1F-Ser-NH2 | 1703 |
| I-2219 | Ac-PL3-Asp-Npg-B5-Asp-3COOHF-Gln-Ala-Phe-Leu-PyrS2-2F3MeF-34C1F-Ala-NH2 | 1704 |
| I-2220 | Ac-PL3-Asp-Npg-B5-Asp-3COOHF-Ala-Ala-Phe-Leu-PyrS2-3Thi-4C1F-Gln-NH2 | 1705 |
| I-2221 | Ac-PL3-Asp-Npg-B5-Asp-3COOHF-Gln-Ala-Phe-Val-PyrS2-3Thi-4C1F-Ala-NH2 | 1706 |
| I-2222 | Ac-PL3-Asp-Npg-B5-Asp-3COOHF-Ala-Ala-Phe-Leu-PyrS2-2F3MeF-4C1F-Ser-NH2 | 1707 |
| I-2223 | Ac-PL3-Asp-Npg-B5-Asp-3COOHF-Gln-Ala-Phe-Leu-PyrS2-2F3MeF-4C1F-Ala-NH2 | 1708 |
| I-2224 | Ac-PL3-Asp-Npg-B5-Asp-3COOHF-Ala-Ala-Phe-Leu-PyrS2-3Thi-3C1F-Gln-NH2 | 1709 |
| I-2225 | Ac-PL3-Asp-Npg-B5-Asp-3COOHF-Gln-Ala-Phe-Val-PyrS2-3Thi-3C1F-Ala-NH2 | 1710 |
| I-2226 | Ac-PL3-Asp-Npg-B5-Asp-3COOHF-Ala-Ala-Phe-Leu-PyrS2-2F3MeF-3C1F-Ser-NH2 | 1711 |
| I-2227 | Ac-PL3-Asp-Npg-B5-Asp-3COOHF-Gln-Ala-Phe-Leu-PyrS2-2F3MeF-3C1F-Ala-NH2 | 1712 |
| I-2228 | Ac-MePro-Asp-Npg-Aib-Asp-Asp-Ala-Ala-Phe-Gln-Aib-3thi-BztA-His-NH2 | 1713 |
| I-2229 | Ac-PL3-Asp-Npg-B5-Asp-Asp-Ala-Ala-Phe-Gln-PyrS2-3thi-BztA-His-Lys-NH2 | 1714 |
| I-2230 | Ac-PL3-Asp-Npg-B5-Asp-Asp-Ala-Ala-Phe-Gln-PyrS2-3thi-BztA-His-Lys-NH2 | 1714 |
| I-2231 | Ac-MePro-Asp-Npg-Aib-Asp-Asp-Ala-Ala-Phe-Gln-Aib-3thi-BztA-His-Lys-NH2 | 1715 |
| I-2232 | Ac-MePro-His-Gln-Aib-BztA-3thi-Ala-Phe-Asp-Npg-Aib-Asp-Ala-Asp-Lys-NH2 | 1716 |
| I-2233 | Ac-PL3-Asp-Npg-B5-Asp-[NdiMeButC]GA-Ala-Ala-Phe-Leu-PyrS2-3Thi-BztA-Gln- | 1717 |
| | NH2 | |
| I-2234 | Ac-PL3-Asp-Npg-B5-Asp-[Me2NPr]GA-Ala-Ala-Phe-Leu-PyrS2-3Thi-BztA-Gln-NH2 | 1718 |
| I-2235 | Ac-PL3-Asp-Npg-B5-Asp-[4AcMePip]GA-Ala-Ala-Phe-Leu-PyrS2-3Thi-BztA-Gln- | 1719 |
| | NH2 | |
| I-2236 | Ac-PL3-Asp-Npg-B5-Asp-[4CF3PhAc]GA-Ala-Ala-Phe-Leu-PyrS2-3Thi-BztA-Gln- | 1720 |
| | NH2 | |
| I-2237 | Ac-PL3-Asp-Npg-B5-Asp-[Pic]GA-Ala-Ala-Phe-Leu-PyrS2-3Thi-BztA-Gln-NH2 | 1721 |
| I-2238 | Ac-PL3-Asp-Npg-B5-Asp-[MeMorphBz]GA-Ala-Ala-Phe-Leu-PyrS2-3Thi-BztA-Gln- | 1722 |
| | NH2 | |
| I-2239 | Ac-PL3-Asp-Npg-B5-Asp-[MorphAc]GA-Ala-Ala-Phe-Leu-PyrS2-3Thi-BztA-Gln- | 1723 |
| | NH2 | |
| I-2240 | Ac-PL3-Asp-Npg-B5-Asp-[MePipAc]GA-Ala-Ala-Phe-Leu-PyrS2-3Thi-BztA-Gln-NH2 | 1724 |
| I-2241 | Ac-PL3-Asp-Npg-B5-Asp-[PfPhAc]GA-Ala-Ala-Phe-Leu-PyrS2-3Thi-BztA-Gln-NH2 | 1725 |
| I-2242 | Ac-PL3-Asp-Npg-B5-Asp-[PfBz]GA-Ala-Ala-Phe-Leu-PyrS2-3Thi-BztA-Gln-NH2 | 1726 |
| I-2243 | Ac-PL3-Asp-Npg-B5-Asp-[NdiMeButC]GAbu-Ala-Ala-Phe-Leu-PyrS2-3Thi-BztA- | 1727 |
| | Gln-NH2 | |
| I-2244 | Ac-PL3-Asp-Npg-B5-Asp-[Me2NPr]GAbu-Ala-Ala-Phe-Leu-PyrS2-3Thi-BztA-Gln- | 1728 |
| | NH2 | |
| I-2245 | Ac-PL3-Asp-Npg-B5-Asp-[4AcMePip]GAbu-Ala-Ala-Phe-Leu-PyrS2-3Thi-BztA-Gln- | 1729 |
| | NH2 | |
| I-2246 | Ac-PL3-Asp-Npg-B5-Asp-[4CF3PhAc]GAbu-Ala-Ala-Phe-Leu-PyrS2-3Thi-BztA-Gln- | 1730 |
| | NH2 | |
| I-2247 | Ac-PL3-Asp-Npg-B5-Asp-[Pic]GAbu-Ala-Ala-Phe-Leu-PyrS2-3Thi-BztA-Gln-NH2 | 1731 |
| I-2248 | Ac-PL3-Asp-Npg-B5-Asp-[MeMorphBz]GAbu-Ala-Ala-Phe-Leu-PyrS2-3Thi-BztA- | 1732 |
| | Gln-NH2 | |
| I-2249 | Ac-PL3-Asp-Npg-B5-Asp-[MorphAc]GAbu-Ala-Ala-Phe-Leu-PyrS2-3Thi-BztA-Gln- | 1733 |
| | NH2 | |
| I-2250 | Ac-PL3-Asp-Npg-B5-Asp-[MePipAc]GAbu-Ala-Ala-Phe-Leu-PyrS2-3Thi-BztA-Gln- | 1734 |
| | NH2 | |
| I-2251 | Ac-PL3-Asp-Npg-B5-Asp-[PfPhAc]GAbu-Ala-Ala-Phe-Leu-PyrS2-3Thi-BztA-Gln- | 1735 |
| | NH2 | |
| I-2252 | Ac-PL3-Asp-Npg-B5-Asp-[PfBz]GAbu-Ala-Ala-Phe-Leu-PyrS2-3Thi-BztA-Gln-NH2 | 1736 |
| I-2253 | ClAc-PL3-Asp-Npg-B5-Asp-3COOHF-Ala-Ala-Phe-Leu-PyrS2-2C1F-BztA-Ala-NH2 | 1737 |
| I-2254 | Ac-PL3-Asp-Npg-B5-Asp-3COOHF-Ala-Ala-Phe-Leu-PyrS2-[Ph]3SF-3Thi-Gln-NH2 | 1738 |
| I-2255 | Ac-PL3-Asp-Npg-B5-Asp-3COOHF-Ala-Ala-Phe-Leu-PyrS2-[Ph]3SF-Cba-Gln-NH2 | 1739 |
| I-2256 | Ac-PL3-Asp-Npg-B5-Asp-3COOHF-Ala-Ala-Phe-Leu-PyrS2-[Ph]3SF-2F3MeF-Gln- | 1740 |
| | NH2 | |
| I-2257 | Ac-PL3-Asp-Npg-B5-Asp-3COOHF-Ala-Ala-Phe-Leu-PyrS2-3Thi-BztA-Ala-NH2 | 1741 |
| I-2258 | Ac-PL3-RbOHAsp-Npg-B5-Asp-3COOHF-Ala-Ala-Phe-Leu-PyrS2-3Thi-BztA-Ala- | 1742 |
| | NH2 | |
| I-2259 | Ac-PL3-Asp-Npg-B5-Asp-3COOHF-Ala-Ala-Phe-Phe-PyrS2-3Thi-BztA-Ala-NH2 | 1743 |
| I-2260 | Ac-PL3-Asp-Npg-B5-Asp-3COOHF-Ala-Ala-3FF-Leu-PyrS2-3Thi-BztA-Ala-NH2 | 1744 |
| I-2261 | Ac-PL3-Asp-Npg-B5-Asp-3COOHF-Ala-Ala-Phe-Leu-PyrS2-3FF-BztA-Ala-NH2 | 1745 |
| I-2262 | Ac-PL3-Asp-Npg-B5-Asp-3COOHF-Ala-Ala-4FF-Leu-PyrS2-3Thi-BztA-Ala-NH2 | 1746 |
| I-2263 | Ac-PL3-Asp-Npg-B5-Asp-3COOHF-Ala-Ala-Phe-Leu-PyrS2-4FF-BztA-Ala-NH2 | 1747 |
| I-2264 | Ac-HypEs5-Asp-Npg-B5-Asp-TfeGA-Ala-Ala-Phe-Leu-PyrS2-3Thi-BztA-Gln-NH2 | 1748 |
| I-2265 | Ac-HypEs5-Asp-Npg-B5-Asp-TfeGA-Ala-Ala-Phe-Leu-PyrS2-3Thi-BztA-Gln-NH2 | 1748 |
| I-2266 | ProSAm3-Asp-Npg-B5-Asp-TfeGA-Ala-Ala-Phe-Leu-PyrS2-3Thi-BztA-Gln-NH2 | 1749 |
| I-2267 | ProSAm3-Asp-Npg-B5-Asp-TfeGA-Ala-Ala-Phe-Leu-PyrS2-3Thi-BztA-Gln-NH2 | 1749 |
| I-2268 | Ac-PL3-Asp-Npg-B5-Asp-3COOHF-Ala-Ala-Phe-Leu-PyrS2-3Thi-BztA-Asn-Ala-Ala- | 1750 |
| | Ala-Ala-Ala-NH2 | |
| I-2269 | Ac-PL3-Asp-Npg-B5-Asp-3COOHF-Ala-Ala-Phe-Leu-PyrS2-3Thi-BztA-Asn-Ala-Ala- | 1751 |
| | Ala-Ala-Ala-Ala-Ala-NH2 | |
| I-2270 | Ac-PL3-Asp-Npg-B5-Asp-3COOHF-Ala-Ala-Phe-Leu-PyrS2-3Thi-BztA-Asn-Ala-Ala- | 1752 |
| | Ala-Ala-Ala-Ala-Ala-Ala-Ala-NH2 | |
| I-2271 | Ac-PL3-Asp-Npg-B5-Asp-3COOHF-Ala-Ala-Phe-Leu-PyrS2-3Thi-BztA-Asn-Ala-Aib- | 1753 |
| | Ala-NH2 | |
| I-2272 | Ac-PL3-Asp-Npg-B5-Asp-3COOHF-Ala-Ala-Phe-Leu-PyrS2-3Thi-BztA-Asn-Ala-Aib- | 1754 |
| | Ala-Aib-Ala-NH2 | |
| I-2273 | Ac-PL3-Asp-Npg-B5-Asp-3COOHF-Ala-Ala-Phe-Leu-PyrS2-3Thi-BztA-Asn-Ala-Aib- | 1755 |
| | Ala-Aib-Ala-Aib-Ala-NH2 | |
| I-2274 | Ac-PL3-Asp-Npg-B5-Asp-3COOHF-Ala-Ala-Phe-Leu-PyrS2-3Thi-BztA-Asn-Leu-NH2 | 1756 |
| I-2275 | Ac-PL3-Asp-Npg-B5-Asp-3COOHF-Ala-Ala-Phe-Leu-PyrS2-3Thi-BztA-Asn-Leu-Leu- | 1757 |
| | Leu-NH2 | |
| I-2276 | Ac-PL3-Asp-Npg-B5-Asp-3COOHF-Ala-Ala-Phe-Leu-PyrS2-3Thi-BztA-Asn-Leu-Leu- | 1757 |
| | Leu-NH2 | |
| I-2277 | Ac-PL3-Asp-Npg-B5-Asp-3COOHF-Ala-Ala-Phe-Leu-PyrS2-3Thi-BztA-Asn-Ala-Leu- | 1758 |
| | Leu-NH2 | |
| I-2278 | Ac-PL3-Asp-Npg-B5-Asp-3COOHF-Ala-Ala-Phe-Leu-PyrS2-3Thi-BztA-Asn-Ala-Leu- | 1758 |
| | Leu-NH2 | |
| I-2279 | Ac-PL3-Asp-Npg-B5-Asp-3COOHF-Ala-Ala-Phe-Leu-PyrS2-3Thi-BztA-Asn-Leu-Leu- | 1759 |
| | Leu-Leu-Leu-NH2 | |
| I-2280 | Ac-PL3-Asp-Npg-B5-Asp-3COOHF-Ala-Ala-Phe-Leu-PyrS2-3Thi-BztA-Asn-Leu-Leu- | 1759 |
| | Leu-Leu-Leu-NH2 | |
| I-2281 | Ac-PL3-Asp-Npg-B5-Asp-3COOHF-Ala-Ala-Phe-Leu-PyrS2-3Thi-BztA-Asn-Ala-Leu- | 1760 |
| | Ala-Leu-Ala-NH2 | |
| I-2282 | Ac-PL3-Asp-Npg-B5-Asp-3COOHF-Ala-Ala-Phe-Leu-PyrS2-3Thi-BztA-Asn-Ala-Leu- | 1760 |
| | Ala-Leu-Ala-NH2 | |
| I-2283 | Ac-PL3-Asp-Npg-B5-Asp-3COOHF-Ala-Ala-Phe-Leu-PyrS2-3Thi-BztA-Asn-Ala-Leu- | 1761 |
| | Ala-Leu-Ala-Leu-Ala-NH2 | |
| I-2284 | Ac-PL3-Asp-Npg-B5-Asp-3COOHF-Ala-Ala-Phe-Leu-PyrS2-3Thi-BztA-Asn-Ala-Leu- | 1761 |
| | Ala-Leu-Ala-Leu-Ala-NH2 | |
| I-2285 | BrAc-PL3-Asp-Npg-B5-Asp-Asp-Ala-Ala-Phe-Gln-PyrS2-3Thi-BztA-His-NH2 | 1762 |
| I-2286 | BrAc-PL3-Asp-Npg-B5-Asp-3COOHF-Ala-Ala-Phe-Leu-PyrS2-2C1F-BztA-Ala-NH2 | 1763 |
| I-2287 | Ac-PL3-Asp-Npg-B5-Asp-3COOHF-Ala-Ala-Phe-Leu-PyrS2-3Thi-3Br4FF-Gln-NH2 | 1764 |
| I-2288 | Ac-PL3-Asp-Npg-B5-Asp-3COOHF-Gln-Ala-Phe-Val-PyrS2-3Thi-3Br4FF-Ala-NH2 | 1765 |
| I-2289 | Ac-PL3-Asp-Npg-B5-Asp-3COOHF-Ala-Ala-Phe-Leu-PyrS2-2F3MeF-3Br4FF-Ser- | 1766 |
| | NH2 | |
| I-2290 | Ac-PL3-Asp-Npg-B5-Asp-3COOHF-Gln-Ala-Phe-Leu-PyrS2-2F3MeF-3Br4FF-Ala- | 1767 |
| | NH2 | |
| I-2291 | Ac-PL3-Asp-Npg-B5-Asp-3COOHF-Ala-Ala-Phe-Leu-PyrS2-3Thi-34MeF-Gln-NH2 | 1768 |
| I-2292 | Ac-PL3-Asp-Npg-B5-Asp-3COOHF-Gln-Ala-Phe-Val-PyrS2-3Thi-34MeF-Ala-NH2 | 1769 |
| I-2293 | Ac-PL3-Asp-Npg-B5-Asp-3COOHF-Ala-Ala-Phe-Leu-PyrS2-2F3MeF-34MeF-Ser- | 1770 |
| | NH2 | |
| I-2294 | Ac-PL3-Asp-Npg-B5-Asp-3COOHF-Gln-Ala-Phe-Leu-PyrS2-2F3MeF-34MeF-Ala- | 1771 |
| | NH2 | |
| I-2295 | Ac-PL3-Asp-Npg-B5-Asp-3COOHF-Ala-Ala-Phe-Leu-PyrS2-3Thi-5IndA-Gln-NH2 | 1772 |
| I-2296 | Ac-PL3-Asp-Npg-B5-Asp-3COOHF-Gln-Ala-Phe-Val-PyrS2-3Thi-5IndA-Ala-NH2 | 1773 |
| I-2297 | Ac-PL3-Asp-Npg-B5-Asp-3COOHF-Ala-Ala-Phe-Leu-PyrS2-2F3MeF-5IndA-Ser-NH2 | 1774 |
| I-2298 | Ac-PL3-Asp-Npg-B5-Asp-3COOHF-Gln-Ala-Phe-Leu-PyrS2-2F3MeF-5IndA-Ala-NH2 | 1775 |
| I-2299 | Ac-PL3-Asp-Npg-B5-Asp-3COOHF-[NHEt]GlnR-Ala-Phe-Leu-PyrS2-2F3MeF-BztA- | 1776 |
| | NHEt]GlnR-NH2 | |
| I-2300 | Ac-PL3-Asp-Npg-B5-Asp-3COOHF-[NMe2]GlnR-Ala-Phe-Leu-PyrS2-2F3MeF-BztA- | 1777 |
| | INMe2]GlnR-NH2 | |
| I-2301 | Ac-PL3-Asp-Npg-B5-Asp-3COOHF-[Me2diaminobutane]GlnR-Ala-Phe-Val-PyrS2- | 1778 |
| | 3Thi-BztA-Ala-NH2 | |
| I-2302 | Ac-PL3-Asp-Npg-B5-Asp-3COOHF-[NHEt]GlnR-Ala-Phe-Val-PyrS2-3Thi-BztA-Ala- | 1779 |
| | NH2 | |
| I-2303 | Ac-PL3-Asp-Npg-B5-Asp-3COOHF-[NMe2]GlnR-Ala-Phe-Val-PyrS2-3Thi-BztA-Ala- | 1780 |
| | NH2 | |
| I-2304 | CO2Bu-PL3-Asp-Npg-B5-Asp-3COOHF-Ala-Ala-Phe-Leu-PyrS2-2C1F-BztA-Ala-NH2 | 1781 |
| I-2305 | CO2iBu-PL3-Asp-Npg-B5-Asp-3COOHF-Ala-Ala-Phe-Leu-PyrS2-2C1F-BztA-Ala- | 1782 |
| | NH2 | |
| I-2306 | BzAm3Oallyl-Asp-Npg-B5-Asp-Asp-Ala-Ala-Phe-Asn-PyrS2-3Thi-BztA-Gln-NH2 | 1783 |
| I-2307 | Ac-PL3-Asp-Npg-B5-Asp-3COOHF-Ala-Ala-Phe-Leu-PyrS2-3Thi-3BrF-Gln-NH2 | 1784 |
| I-2308 | Ac-PL3-Asp-Npg-B5-Asp-3COOHF-Gln-Ala-Phe-Val-PyrS2-3Thi-3BrF-Ala-NH2 | 1785 |
| I-2309 | Ac-PL3-Asp-Npg-B5-Asp-3COOHF-Ala-Ala-Phe-Leu-PyrS2-3Thi-2NapA-Gln-NH2 | 1786 |
| I-2310 | Ac-PL3-Asp-Npg-B5-Asp-3COOHF-Gln-Ala-Phe-Val-PyrS2-3Thi-2NapA-Ala-NH2 | 1787 |
| I-2311 | Ac-PL3-Asp-Npg-B5-Asp-3COOHF-Ala-Ala-Phe-Leu-PyrS2-3Thi-RbMe2NapA-Gln- | 1788 |
| | NH2 | |
| I-2312 | Ac-PL3-Asp-Npg-B5-Asp-3COOHF-Gln-Ala-Phe-Val-PyrS2-3Thi-RbMe2NapA-Ala- | 1789 |
| | NH2 | |
| I-2313 | Ac-PL3-Asp-Npg-B5-Asp-3COOHF-Ala-Ala-Phe-Leu-PyrS2-3Thi-SbMe2NapA-Gln- | 1790 |
| | NH2 | |
| I-2314 | Ac-PL3-Asp-Npg-B5-Asp-3COOHF-Gln-Ala-Phe-Val-PyrS2-3Thi-SbMe2NapA-Ala- | 1791 |
| | NH2 | |
| I-2315 | Ac-PL3-Asp-Npg-B5-Asp-3COOHF-[Morph]GlnR-Ala-Phe-Leu-PyrS2-2F3MeF-BztA- | 1792 |
| | Ala-NH2 | |
| I-2316 | Ac-PL3-Asp-Npg-B5-Asp-3COOHF-[TfePpz]GlnR-Ala-Phe-Leu-PyrS2-2F3MeF-BztA- | 1793 |
| | Ala-NH2 | |
| I-2317 | Ac-PL3-Asp-Npg-B5-Asp-3COOHF-[4Pippip]GlnR-Ala-Phe-Leu-PyrS2-2F3MeF- | 1794 |
| | BztA-Ala-NH2 | |
| I-2318 | Ac-PL3-Asp-Npg-B5-Asp-3COOHF-[2OxoPpz]GlnR-Ala-Phe-Leu-PyrS2-2F3MeF- | 1795 |
| | BztA-Ala-NH2 | |
| I-2319 | Ac-PL3-Asp-Npg-B5-Asp-3COOHF-[AcPpz]GlnR-Ala-Phe-Leu-PyrS2-2F3MeF-BztA- | 1796 |
| | Ala-NH2 | |
| I-2320 | Ac-PL3-Asp-Npg-B5-Asp-3COOHF-[isoindoline]GlnR-Ala-Phe-Leu-PyrS2-2F3MeF- | 1797 |
| | BztA-Ala-NH2 | |
| I-2321 | Ac-PL3-Asp-Npg-B5-Asp-3COOHF-[EtSO2Ppz]GlnR-Ala-Phe-Leu-PyrS2-2F3MeF- | 1798 |
| | BztA-Ala-NH2 | |
| I-2322 | Ac-PL3-Asp-Npg-B5-Asp-3COOHF-[bismethoxyethylamine]GlnR-Ala-Phe-Leu-PyrS2- | 1799 |
| | 2F3MeF-BztA-Ala-NH2 | |
| I-2323 | Ac-PL3-Asp-Npg-B5-Asp-3COOHF-[Me2NPrPip]GlnR-Ala-Phe-Leu-PyrS2-2F3MeF- | 1800 |
| | BztA-Ala-NH2 | |
| I-2324 | CO2Me-PL3-Asp-Npg-B5-Asp-3COOHF-Ala-Ala-Phe-Leu-PyrS2-2C1F-BztA-Ala- | 1801 |
| | NH2 | |
| I-2325 | CO2Hex-PL3-Asp-Npg-B5-Asp-3COOHF-Ala-Ala-Phe-Leu-PyrS2-2C1F-BztA-Ala- | 1802 |
| | NH2 | |
| I-2326 | CO2Ph-PL3-Asp-Npg-B5-Asp-Asp-Ala-Ala-Phe-Gln-PyrS2-3Thi-BztA-His-NH2 | 1803 |
| I-2327 | CO2Ph-PL3-Asp-Npg-B5-Asp-3COOHF-Ala-Ala-Phe-Leu-PyrS2-2C1F-BztA-Ala-NH2 | 1804 |
| I-2328 | Ac-HypBzEs3OAllyl-Asp-Npg-B5-Asp-TfeGA-Ala-Ala-Phe-Leu-PyrS2-3Thi-BztA- | 1805 |
| | Gln-NH2 | |
| I-2329 | Ac-PL3-[CH2CMe2CO2H]TriAzDap-Npg-B5-Asp-Asp-Ala-Ala-Phe-Asn-PyrS2-3Thi- | 1806 |
| | BztA-Gln-NH2 | |
| I-2330 | Ac-PL3-[CMe2CO2H]TriAzDap-Npg-B5-Asp-Asp-Ala-Ala-Phe-Asn-PyrS2-3Thi- | 1807 |
| | BztA-Gln-NH2 | |
| I-2331 | Ac-PL3-Glu-Npg-B5-Asn-Thr-Ala-Ala-Phe-Asn-PyrS2-3Thi-BztA-Gln-NH2 | 1808 |
| I-2332 | Ac-PL3-Glu-Npg-B5-Asn-Thr-Ala-Ala-Phe-Asn-PyrS2-3Thi-BztA-Gln-NH2 | 1808 |
| I-2333 | Ac-PL3-Asn-Npg-B5-Asn-Thr-Ala-Ala-Phe-Asn-PyrS2-3Thi-BztA-Gln-NH2 | 1809 |
| I-2334 | Ac-PL3-Asp-Npg-B5-Asn-Thr-Ala-Ala-Phe-Thr-PyrS2-3Thi-BztA-Ala-NH2 | 1810 |
| I-2335 | Ac-PL3-Asp-Npg-B5-Asn-3COOHF-Ala-Ala-Phe-Asn-PyrS2-3Thi-BztA-Gln-NH2 | 1811 |
| I-2336 | Ac-PL3-Asp-Npg-B5-Asn-3COOHF-Ala-Ala-Phe-Thr-PyrS2-3Thi-BztA-Gln-NH2 | 1812 |
| I-2337 | Ac-PL3-Asp-Npg-B5-Asn-Thr-Ala-Ala-Phe-Thr-PyrS2-3Thi-BztA-His-NH2 | 1813 |
| I-2338 | Ac-PL3-Asp-Npg-B5-Asn-Thr-Aib-Ala-Phe-Asn-PyrS2-3Thi-BztA-Gln-NH2 | 1814 |
| I-2339 | Ac-PL3-Asp-Npg-B5-Asn-Thr-Ala-Aib-Phe-Asn-PyrS2-3Thi-BztA-Gln-NH2 | 1815 |
| I-2340 | Ac-PL3-Asn-Cha-B5-Asp-3COOHF-iPrLys-Ala-Phe-Leu-PyrS2-Phe-BztA-Gln-NH2 | 1816 |
| I-2341 | Ac-PL3-Asn-Cha-B5-Asp-TfeGA-iPrLys-Ala-Phe-Leu-PyrS2-Phe-BztA-Gln-NH2 | 1817 |
| I-2342 | Ac-PL3-Asn-Cha-B5-Asp-3cbmf-iPrLys-Ala-Phe-Leu-PyrS2-Phe-BztA-Gln-NH2 | 1818 |
| I-2343 | Ac-PL3-Asn-Cha-B5-Asp-3cbmf-iPrLys-Ala-Phe-Leu-PyrS2-Phe-BztA-Gln-NH2 | 1818 |
| I-2344 | Ac-PL3-Asn-Cha-B5-Asp-Hse-iPrLys-Ala-Phe-Leu-PyrS2-Phe-BztA-Gln-NH2 | 1819 |
| I-2345 | Ac-PL3-Asn-Cha-B5-Asp-Hse-iPrLys-Ala-Phe-Leu-PyrS2-Phe-BztA-Gln-NH2 | 1819 |
| I-2346 | Ac-PL3-Asn-Cha-B5-Asp-Lys-iPrLys-Ala-Phe-Leu-PyrS2-Phe-BztA-Gln-NH2 | 1820 |
| I-2347 | Ac-PL3-Asn-Cha-B5-Asp-Lys-iPrLys-Ala-Phe-Leu-PyrS2-Phe-BztA-Gln-NH2 | 1820 |
| I-2348 | Ac-PL3-Asn-Cha-B5-Asp-His-iPrLys-Ala-Phe-Leu-PyrS2-Phe-BztA-Gln-NH2 | 1821 |
| I-2349 | Ac-PL3-Asn-Cha-B5-Asp-His-iPrLys-Ala-Phe-Leu-PyrS2-Phe-BztA-Gln-NH2 | 1821 |
| I-2350 | Ac-PL3-Asn-Cha-B5-Asp-Gln-iPrLys-Ala-Phe-Leu-PyrS2-Phe-BztA-Lys-NH2 | 1822 |
| I-2351 | Ac-PL3-Asn-Cha-B5-Asp-Gln-iPrLys-Ala-Phe-Leu-PyrS2-Phe-BztA-His-NH2 | 1823 |
| I-2352 | Ac-PL3-Asn-Cha-B5-Asp-Gln-iPrLys-Ala-Phe-Leu-PyrS2-Phe-BztA-Ala-NH2 | 1824 |
| I-2353 | Ac-PL3-Hse-Cha-B5-Asp-Gln-iPrLys-Ala-Phe-Leu-PyrS2-Phe-BztA-Gln-NH2 | 1825 |
| I-2354 | Ac-PL3-Asn-Cha-B5-Asp-Gln-Hse-Ala-Phe-Leu-PyrS2-Phe-BztA-Gln-NH2 | 1826 |
| I-2355 | Ac-PL3-Asn-Cha-B5-Asp-Gln-Hse-Ala-Phe-Leu-PyrS2-Phe-BztA-Gln-NH2 | 1826 |
| I-2356 | Ac-PL3-[Et]AspE-Npg-B5-Asp-Asp-Ala-Ala-Phe-Leu-PyrS2-3Thi-BztA-Gln-NH2 | 1827 |
| I-2357 | Ac-PL3-Asp-Npg-B5-[Et]AspE-Asp-Ala-Ala-Phe-Leu-PyrS2-3Thi-BztA-Gln-NH2 | 1828 |
| I-2358 | Ac-PL3-Asp-Npg-B5-Asp-[Et]AspE-Ala-Ala-Phe-Leu-PyrS2-3Thi-BztA-Gln-NH2 | 1829 |
| I-2359 | Ac-PL3-[Me]AspE-Npg-B5-Asp-Asp-Ala-Ala-Phe-Leu-PyrS2-3Thi-BztA-Gln-NH2 | 1830 |
| I-2360 | Ac-PL3-Asp-Npg-B5-[Me]AspE-Asp-Ala-Ala-Phe-Leu-PyrS2-3Thi-BztA-Gln-NH2 | 1831 |
| I-2361 | Ac-PL3-Asp-Npg-B5-Asp-[Me]AspE-Ala-Ala-Phe-Leu-PyrS2-3Thi-BztA-Gln-NH2 | 1832 |
| I-2362 | CO2Me-PL3-Asp-Npg-B5-Asp-Asp-Ala-Ala-Phe-Gln-PyrS2-3Thi-BztA-His-NH2 | 1833 |
| I-2363 | CO2Bu-PL3-Asp-Npg-B5-Asp-Asp-Ala-Ala-Phe-Gln-PyrS2-3Thi-BztA-His-NH2 | 1834 |
| I-2364 | CO2iBu-PL3-Asp-Npg-B5-Asp-Asp-Ala-Ala-Phe-Gln-PyrS2-3Thi-BztA-His-NH2 | 1835 |
| I-2365 | Ac-PL3-Asp-Npg-B5-Asp-[Et]GluE-Ala-Ala-Phe-Leu-PyrS2-3Thi-BztA-Gln-NH2 | 1836 |
| I-2366 | Ac-PL3-Asp-Npg-B5-Asp-[Me]GluE-Ala-Ala-Phe-Leu-PyrS2-3Thi-BztA-Gln-NH2 | 1837 |
| I-2367 | Ac-PL3-Asp-Npg-B5-Asp-[CH2CH2CO2H]TriAzDap-Gln-Ala-Phe-Leu-PyrS2- | 1838 |
| | 2F3MeF-BztA-Ala-NH2 | |
| I-2368 | Ac-PL3-Asp-Npg-B5-Asp-[CH2CMe2CO2H]TriAzDap-Gln-Ala-Phe-Leu-PyrS2- | 1839 |
| | 2F3MeF-BztA-Ala-NH2 | |
| I-2369 | Ac-PL3-Asp-Npg-B5-Asp-[CMe2CO2H]TriAzDap-Gln-Ala-Phe-Leu-PyrS2-2F3MeF- | 1840 |
| | BztA-Ala-NH2 | |
| I-2370 | Ac-PL3-Asp-Npg-B5-Asp-[CH2CH2CO2H]TriAzDap-Ala-Ala-Phe-Leu-PyrS2- | 1841 |
| | 2F3MeF-BztA-Ser-NH2 | |
| I-2371 | Ac-PL3-Asp-Npg-B5-Asp-[CH2CH2CO2H]TriAzDap-Ala-Ala-Phe-Leu-PyrS2- | 1841 |
| | 2F3MeF-BztA-Ser-NH2 | |
| I-2372 | Ac-PL3-Asp-Npg-B5-Asp-[CH2CMe2CO2H]TriAzDap-Ala-Ala-Phe-Leu-PyrS2- | 1842 |
| | 2F3MeF-BztA-Ser-NH2 | |
| I-2373 | Ac-PL3-Asp-Npg-B5-Asp-[CH2CO2H]TriAzDap-Ala-Ala-Phe-Leu-PyrS2-2F3MeF- | 1843 |
| | BztA-Ser-NH2 | |
| I-2374 | Ac-PL3-Asp-Npg-B5-Asp-[CMe2CO2H]TriAzDap-Ala-Ala-Phe-Leu-PyrS2-2F3MeF- | 1844 |
| | BztA-Ser-NH2 | |
| I-2375 | Ac-PL3-Asp-Npg-B5-Asp-Asp-Ala-Ala-Phe-Leu-PyrS2-3Thi-BztA-[3C]TriAzLys-NH2 | 1845 |
| I-2376 | BzAm3OAllyl-Asp-Npg-B5-Asp-TfeGA-Ala-Ala-Phe-Leu-PyrS2-3Thi-BztA-Gln-NH2 | 1846 |
| I-2377 | ProBzAm3OAllyl-Asp-Npg-B5-Asp-TfeGA-Ala-Ala-Phe-Leu-PyrS2-3Thi-BztA-Gln- | 1847 |
| | NH2 | |
| I-2378 | Ac-PL3-Asp-Npg-B5-Asp-3COOHF-Ala-Ala-Phe-Leu-PyrS2-2F3MeF-BztA-Gln-Leu- | 1848 |
| | Ala-NH2 | |
| I-2379 | Ac-PL3-Asp-Npg-B5-Asp-3COOHF-Ala-Ala-Phe-Leu-PyrS2-2F3MeF-BztA-Gln-Leu- | 1849 |
| | Val-NH2 | |
| I-2380 | Ac-PL3-Asp-Npg-B5-Asp-3COOHF-Ala-Ala-Phe-Leu-PyrS2-2F3MeF-BztA-Gln-Leu- | 1850 |
| | Leu-NH2 | |
| I-2381 | Ac-PL3-Asp-Npg-B5-Asp-3COOHF-Ala-Ala-Phe-Leu-PyrS2-2F3MeF-BztA-Gln-Leu- | 1851 |
| | Phe-NH2 | |
| I-2382 | Ac-PL3-Asp-Npg-B5-Asp-3COOHF-Ala-Ala-Phe-Leu-PyrS2-2F3MeF-BztA-Gln-Leu- | 1852 |
| | Gln-NH2 | |
| I-2383 | Ac-PL3-Asp-Npg-B5-Asp-3COOHF-Ala-Ala-Phe-Leu-PyrS2-2F3MeF-BztA-Gln-Leu- | 1853 |
| | Thr-NH2 | |
| I-2384 | Ac-PL3-Asp-Npg-B5-Asp-3COOHF-Ala-Ala-Phe-Leu-PyrS2-2F3MeF-BztA-Gln-Leu- | 1854 |
| | Ser-NH2 | |
| I-2385 | Ac-PL3-Asp-Npg-B5-Asp-3COOHF-Ala-Ala-Phe-Leu-PyrS2-2F3MeF-BztA-Gln-Leu- | 1855 |
| | Ile-NH2 | |
| I-2386 | Ac-PL3-Asp-Npg-B5-Asp-3COOHF-Ala-Ala-Phe-Leu-PyrS2-2F3MeF-BztA-Gln-Leu- | 1856 |
| | Aib-NH2 | |
| I-2387 | Ac-PL3-Asp-Npg-B5-Asp-3COOHF-Ala-Ala-Phe-Leu-PyrS2-2F3MeF-BztA-Gln-Leu- | 1857 |
| | CyLeu-NH2 | |
| I-2388 | Ac-PL3-Asp-Npg-B5-Asp-3COOHF-Ala-Ala-Phe-Leu-PyrS2-2F3MeF-BztA-Gln-Leu- | 1858 |
| | Cbg-NH2 | |
| I-2389 | Ac-PL3-Asp-Npg-B5-Asp-3COOHF-Ala-Ala-Phe-Leu-PyrS2-2F3MeF-BztA-Gln-Leu- | 1859 |
| | Cpg-NH2 | |
| I-2390 | Ac-PL3-Asp-Npg-B5-Asp-3COOHF-Ala-Ala-Phe-Leu-PyrS2-2F3MeF-BztA-Gln-Leu- | 1860 |
| | Asn-NH2 | |
| I-2391 | Ac-PL3-Asp-Npg-B5-Asp-3COOHF-Ala-Ala-Phe-Leu-PyrS2-2F3MeF-BztA-Gln-Leu- | 1861 |
| | nLeu-NH2 | |
| I-2392 | Ac-PL3-Asp-Npg-B5-Asp-3COOHF-Ala-Ala-Phe-Leu-PyrS2-2F3MeF-BztA-Gln-Leu- | 1862 |
| | Tyr-NH2 | |
| I-2393 | Ac-PL3-Asp-Npg-B5-Asp-3COOHF-Ala-Ala-Phe-Leu-PyrS2-2F3MeF-BztA-Gln-Leu- | 1863 |
| | Arg-NH2 | |
| I-2394 | Ac-PL3-Asp-Npg-B5-Asp-3COOHF-Ala-Ala-Phe-Leu-PyrS2-2F3MeF-BztA-Gln-Leu- | 1864 |
| | Trp-NH2 | |
| I-2395 | Ac-PL3-Asp-Npg-B5-Asp-3COOHF-Ala-Ala-Phe-Leu-PyrS2-2F3MeF-BztA-Gln-Leu- | 1865 |
| | dAla-NH2 | |
| I-2396 | Ac-PL3-Asp-Npg-B5-Asp-3COOHF-Ala-Ala-Phe-Leu-PyrS2-2F3MeF-BztA-Gln-Leu- | 1866 |
| | dLeu-NH2 | |
| I-2397 | Ac-PL3-Asp-Npg-B5-Asp-3COOHF-Ala-Ala-Phe-Leu-PyrS2-2F3MeF-BztA-Gln-Leu- | 1867 |
| | dThr-NH2 | |
| I-2398 | Ac-PL3-Asp-Npg-B5-Asp-3COOHF-Ala-Ala-Phe-Leu-PyrS2-2F3MeF-BztA-Gln-Leu- | 1868 |
| | dGln-NH2 | |
| I-2399 | Ac-PL3-Asp-Npg-B5-Asp-3COOHF-Ala-Ala-Phe-Leu-PyrS2-2F3MeF-BztA-Gln-Ala- | 1869 |
| | Ala-NH2 | |
| I-2400 | Ac-PL3-Asp-Npg-B5-Asp-3COOHF-Ala-Ala-Phe-Leu-PyrS2-2F3MeF-BztA-Gln-Phe- | 1870 |
| | Ala-NH2 | |
| I-2401 | Ac-PL3-Asp-Npg-B5-Asp-3COOHF-Ala-Ala-Phe-Leu-PyrS2-2F3MeF-BztA-Gln-Val- | 1871 |
| | Ala-NH2 | |
| I-2402 | PropynOH-PL3-Asp-Npg-B5-Asp-Asp-Ala-Ala-Phe-Gln-PyrS2-3Thi-BztA-His-NH2 | 1872 |
| I-2403 | PropynOH-PL3-Asp-Npg-B5-Asp-3COOHF-Ala-Ala-Phe-Leu-PyrS2-2C1F-BztA-Ala- | 1873 |
| | NH2 | |
| I-2404 | PAc3OAllyl-Asp-Npg-B5-Asp-TfeGA-Ala-Ala-Phe-Leu-PyrS2-3Thi-BztA-Gln-NH2 | 1874 |
| I-2405 | ProPAc3OAllyl-Asp-Npg-B5-Asp-TfeGA-Ala-Ala-Phe-Leu-PyrS2-3Thi-BztA-Gln- | 1875 |
| | NH2 | |
| I-2406 | Ac-HypPAc3OAllyl-Asp-Npg-B5-Asp-TfeGA-Ala-Ala-Phe-Leu-PyrS2-3Thi-BztA-Gln- | 1876 |
| | NH2 | |
| I-2407 | Ac-PL3-Asp-Npg-B5-Asn-Thr-Aib-Ala-Phe-Arg-PyrS2-3Thi-BztA-Gln-NH2 | 1877 |
| I-2408 | Ac-PL3-Asp-Npg-B5-Asn-Thr-Aib-Ala-Phe-Leu-PyrS2-3Thi-BztA-Gln-NH2 | 1878 |
| I-2409 | Ac-PL3-Asp-Npg-B5-Asn-Thr-Aib-Ala-Phe-Thr-PyrS2-3Thi-BztA-Gln-NH2 | 1879 |
| I-2410 | Ac-PL3-Asp-Npg-B5-Asn-Thr-Aib-Ala-Phe-His-PyrS2-3Thi-BztA-Gln-NH2 | 1880 |
| I-2411 | Ac-PL3-Asp-Npg-B5-Asn-Thr-Aib-Ala-Phe-Asn-PyrS2-3Thi-BztA-Ala-NH2 | 1881 |
| I-2412 | Ac-PL3-Asp-Npg-B5-Asn-Thr-Aib-Ala-Phe-Asn-PyrS2-3Thi-BztA-Phe-NH2 | 1882 |
| I-2413 | Ac-PL3-Asp-Npg-B5-Asn-Thr-Aib-Ala-Phe-Asn-PyrS2-3Thi-BztA-Arg-NH2 | 1883 |
| I-2414 | Ac-PL3-Asp-Npg-B5-Asn-Thr-Aib-Ala-Phe-Asn-PyrS2-3Thi-BztA-His-NH2 | 1884 |
| I-2415 | Ac-PL3-Asp-Npg-B5-Asn-Thr-Aib-Ala-Phe-Asn-PyrS2-3Thi-BztA-Asn-NH2 | 1885 |
| I-2416 | Ac-PL3-Asp-Npg-B5-Asn-Thr-Aib-Ala-Phe-Asn-PyrS2-3Thi-BztA-Thr-NH2 | 1886 |
| I-2417 | Ac-PL3-Asp-Npg-B5-Asn-Thr-Aib-Ala-Phe-Asn-PyrS2-3Thi-34C1F-Gln-NH2 | 1887 |
| I-2418 | Ac-PL3-Asn-Cha-B5-Asp-Gln-Ala-Ala-Phe-Leu-PyrS2-Phe-BztA-Lys-NH2 | 1888 |
| I-2419 | Ac-PL3-Asn-Cha-B5-Asp-Gln-Hse-Ala-Phe-Leu-PyrS2-Phe-BztA-Lys-NH2 | 1889 |
| I-2420 | Ac-PL3-Asn-Cha-B5-Asp-Gln-Asn-Ala-Phe-Leu-PyrS2-Phe-BztA-Lys-NH2 | 1890 |
| I-2421 | Ac-PL3-Asn-Cha-B5-Asp-Gln-Gln-Ala-Phe-Leu-PyrS2-Phe-BztA-Lys-NH2 | 1891 |
| I-2422 | Ac-PL3-Asn-Cha-B5-Asp-Gln-Ser-Ala-Phe-Leu-PyrS2-Phe-BztA-Lys-NH2 | 1892 |
| I-2423 | Ac-PL3-Asn-Cha-B5-Asp-Gln-Thr-Ala-Phe-Leu-PyrS2-Phe-BztA-Lys-NH2 | 1893 |
| I-2424 | Ac-PL3-Asn-Cha-B5-Asp-Gln-Aib-Ala-Phe-Leu-PyrS2-Phe-BztA-Lys-NH2 | 1894 |
| I-2425 | Ac-PL3-Asn-Cha-B5-Asp-Gln-MorphAsn-Ala-Phe-Leu-PyrS2-Phe-BztA-Lys-NH2 | 1895 |
| I-2426 | Ac-PL3-Asn-Cha-B5-Asp-Gln-iPrLys-Ala-Phe-Leu-PyrS2-Phe-BztA-MorphAsn-NH2 | 1896 |
| I-2427 | Ac-PL3-Asn-Cha-B5-Asp-Gln-MorphNva-Ala-Phe-Leu-PyrS2-Phe-BztA-Lys-NH2 | 1897 |
| I-2428 | Ac-PL3-Asn-Cha-B5-Asp-Gln-iPrLys-Ala-Phe-Leu-PyrS2-Phe-BztA-MorphNva-NH2 | 1898 |
| I-2429 | Ac-PL3-Asp-Cha-B5-Asp-Gln-iPrLys-Ala-Phe-Leu-PyrS2-Phe-BztA-Lys-NH2 | 1899 |
| I-2430 | Ac-PL3-Asp-Npg-B5-Asp-5iPr3COOHF-Ala-Ala-Phe-Leu-PyrS2-2F3MeF-BztA-Gln- | 1900 |
| | NH2 | |
| I-2431 | Ac-PL3-Asp-Npg-B5-Asp-5iPr3COOHF-Gln-Ala-Phe-Leu-PyrS2-2F3MeF-BztA-Ala- | 1901 |
| | NH2 | |
| I-2432 | Ac-PL3-Asp-Npg-B5-Asp-3COOHF-Ala-Ala-Phe-Leu-PyrS2-2F3MeF-BztA-Ser- | 1902 |
| | [3C]TriAzLys-NH2 | |
| I-2433 | Ac-PL3-Hse-Npg-B5-Hse-Glu-Ala-Ala-4BrF-Leu-PyrS2-Phe-BztA-Asn-NH2 | 1903 |
| I-2434 | Ac-PL3-Asn-Npg-B5-Asn-Glu-Gln-Ala-Phe-Leu-PyrS2-Phe-BztA-Gln-NH2 | 1904 |
| I-2435 | Ac-PL3-Asn-Npg-B5-Asn-Glu-Gln-Ala-Phe-Leu-S8-Phe-BztA-Gln-NH2 | 1905 |
| I-2436 | Ac-PL3-Hse-Npg-B5-Asn-Glu-Ala-Ala-Phe-Leu-PyrS2-Phe-BztA-Asn-NH2 | 1906 |
| I-2437 | Ac-PL3-Asn-Npg-B5-Asn-Glu-Ser-Ala-Phe-Leu-PyrS2-Phe-BztA-Gln-NH2 | 1907 |
| I-2438 | Ac-PL3-Hse-Npg-B5-Asp-His-Ala-Ala-Phe-Thr-PyrS2-Phe-BztA-Gln-NH2 | 1908 |
| I-2439 | Ac-PL3-Hse-Npg-B5-Asp-Gln-Ala-Ala-Phe-His-PyrS2-Phe-BztA-Gln-NH2 | 1909 |
| I-2440 | Ac-PL3-Asn-Npg-B5-Asp-Gln-Ala-Ala-Phe-His-PyrS2-Phe-BztA-Gln-NH2 | 1910 |
| I-2441 | Ac-PL3-Hse-Npg-B5-Asp-Arg-Ala-Ala-Phe-Thr-PyrS2-Phe-BztA-Gln-NH2 | 1911 |
| I-2442 | Ac-PL3-Asn-Npg-B5-Asp-His-Ala-Ala-Phe-Leu-PyrS2-Phe-BztA-Gln-NH2 | 1912 |
| I-2443 | Ac-PL3-Hse-Npg-B5-Asp-Gln-Ala-Ala-Phe-Leu-PyrS2-Phe-BztA-Gln-NH2 | 1913 |
| I-2444 | Ac-PL3-Asp-Npg-B5-Asp-3COOHF-Ala-Ala-Phe-Leu-PyrS2-2F3MeF-BztA-Ser- | 1914 |
| | [3C]TriAzdLys-NH2 | |
| I-2445 | Ac-PL3-Asp-Npg-B5-Asp-[CCpCO2H]TriAzDap-Ala-Ala-Phe-Leu-PyrS2-3Thi-BztA- | 1915 |
| | Gln-NH2 | |
| I-2446 | Ac-PL3-Asn-Cha-B5-Asp-Gln-iPrLys-Ala-Phe-Leu-PyrS2-3Thi-BztA-Gln-NH2 | 1916 |
| I-2447 | Ac-PL3-Asn-Cha-B5-Asp-Thr-Hse-Ala-Phe-Leu-PyrS2-Phe-BztA-Gln-NH2 | 1917 |
| I-2448 | Ac-PL3-Asn-Cha-B5-Asp-Thr-iPrLys-Ala-Phe-Leu-PyrS2-Phe-BztA-Gln-NH2 | 1918 |
| I-2449 | Ac-PL3-Asn-Cha-B5-Asp-Thr-Hse-Ala-Phe-Leu-PyrS2-Phe-BztA-Lys-NH2 | 1919 |
| I-2450 | Ac-PL3-Asn-Cha-B5-Asp-Thr-iPrLys-Ala-Phe-Leu-PyrS2-Phe-BztA-Lys-NH2 | 1920 |
| I-2451 | Ac-PL3-Asn-Cha-B5-Asp-Gln-Hse-Ala-Phe-Leu-PyrS2-3Thi-BztA-Gln-NH2 | 1921 |
| I-2452 | Ac-PL3-Asn-Cha-B5-Asp-Gln-iPrLys-Ala-Phe-Leu-PyrS2-3Thi-BztA-Gln-NH2 | 1916 |
| I-2453 | Ac-PL3-Asn-Cha-B5-Asp-Thr-iPrLys-Ala-Phe-Leu-PyrS2-Phe-BztA-Gln-NH2 | 1918 |
| I-2454 | Ac-PL3-Asn-Cha-B5-Asp-Thr-Hse-Ala-Phe-Leu-PyrS2-Phe-BztA-Lys-NH2 | 1919 |
| I-2455 | Ac-PL3-Asn-Cha-B5-Asp-Thr-iPrLys-Ala-Phe-Leu-PyrS2-Phe-BztA-Lys-NH2 | 1920 |
| I-2456 | Ac-PL3-Asp-Npg-B5-Asp-3COOHF-Ala-Ala-Phe-Leu-PyrS2-2F3MeF-SbMeBztA-Gln- | 1922 |
| | NH2 | |
| I-2457 | Ac-PL3-Asp-Npg-B5-Asp-3COOHF-Gln-Ala-Phe-Leu-PyrS2-2F3MeF-SbMeBztA-Ala- | 1923 |
| | NH2 | |
| I-2458 | Ac-PL3-Asp-Npg-B5-Asp-3COOHF-Ala-Ala-Phe-Leu-PyrS2-2F3MeF-RbMeBztA- | 1924 |
| | Gln-NH2 | |
| I-2459 | Ac-PL3-Asp-Npg-B5-Asp-3COOHF-Gln-Ala-Phe-Leu-PyrS2-2F3MeF-RbMeBztA- | 1925 |
| | Ala-NH2 | |
| I-2460 | Ac-PL3-Asp-Npg-B5-Asp-3COOHF-Ala-Ala-Phe-Leu-PyrS2-2F3MeF-2NapA-Gln- | 1926 |
| | NH2 | |
| I-2461 | Ac-PL3-Asp-Npg-B5-Asp-3COOHF-Gln-Ala-Phe-Leu-PyrS2-2F3MeF-2NapA-Ala- | 1927 |
| | NH2 | |
| I-2462 | Ac-PL3-Asp-Npg-B5-Asp-3COOHF-Ala-Ala-Phe-Leu-PyrS2-2F3MeF-RbMe2NapA- | 1928 |
| | Gln-NH2 | |
| I-2463 | Ac-PL3-Asp-Npg-B5-Asp-3COOHF-Gln-Ala-Phe-Leu-PyrS2-2F3MeF-RbMe2NapA- | 1929 |
| | Ala-NH2 | |
| I-2464 | Ac-PL3-Asp-Npg-B5-Asp-3COOHF-Ala-Ala-Phe-Leu-PyrS2-2F3MeF-SbMe2NapA- | 1930 |
| | Gln-NH2 | |
| I-2465 | Ac-PL3-Asp-Npg-B5-Asp-3COOHF-Gln-Ala-Phe-Leu-PyrS2-2F3MeF-SbMe2NapA- | 1931 |
| | Ala-NH2 | |
| I-2466 | Ac-PL3-Asp-Npg-B5-Asp-3COOHF-Ala-Ala-Phe-Leu-PyrS2-2F3MeF-BztA-Me2Gln- | 1932 |
| | Ala-NH2 | |
| I-2467 | Ac-PL3-Asp-Npg-B5-Asp-TfeGA-Ala-Ala-Phe-Leu-PyrS2-3Thi-BztA-Me2Gln-Ala- | 1933 |
| | NH2 | |
| I-2468 | Ac-PL3-Asp-Npg-B5-Asp-3COOHF-Aib-Ala-Phe-Abu-PyrS2-2F3MeF-BztA-Me2Gln- | 1934 |
| | Ala-NH2 | |
| I-2469 | Ac-PL3-Asp-Npg-B5-Asp-3COOHF-Aib-Ala-Phe-Abu-PyrS2-3Thi-BztA-Me2Gln-Ala- | 1935 |
| | NH2 | |
| I-2470 | Ac-PL3-Asp-Npg-B5-Asp-3COOHF-Ala-Ala-Phe-Leu-PyrS2-2F3MeF-BztA- | 1936 |
| | NHEt]GlnR-Ala-NH2 | |
| I-2471 | Ac-PL3-Asp-Npg-B5-Asp-TfeGA-Ala-Ala-Phe-Leu-PyrS2-3Thi-BztA-[NHEt]GlnR- | 1937 |
| | Ala-NH2 | |
| I-2472 | Ac-PL3-Asp-Npg-B5-Asp-3COOHF-Ala-Ala-Phe-Abu-PyrS2-2F3MeF-BztA- | 1938 |
| | INHEt]GlnR-Ala-NH2 | |
| I-2473 | Ac-PL3-Asp-Npg-B5-Asp-3COOHF-Aib-Ala-Phe-Abu-PyrS2-3Thi-BztA-[NHEt]GlnR- | 1939 |
| | Ala-NH2 | |
| I-2474 | Ac-PL3-Asp-Npg-B5-Asp-TfeGA-Ala-Ala-Phe-Leu-PyrS2-3Thi-BztA-[Pip]GlnR-Ala- | 1940 |
| | NH2 | |
| I-2475 | Ac-PL3-Asp-Npg-B5-Asp-3COOHF-Ala-Ala-Phe-Abu-PyrS2-2F3MeF-BztA- | 1941 |
| | [Pip ]GlnR-Ala-NH2 | |
| I-2476 | Ac-PL3-Asp-Npg-B5-Asp-3COOHF-Aib-Ala-Phe-Abu-PyrS2-3Thi-BztA-[Pip]GlnR- | 1942 |
| | Ala-NH2 | |
| I-2477 | Ac-PL3-Asp-Npg-B5-Asn-Thr-Aib-Ala-Phe-Asn-PyrS2-3Thi-BztA-Lys-NH2 | 1943 |
| I-2478 | Ac-PL3-Asp-Npg-B5-Asn-Thr-Aib-Ala-Phe-Asn-PyrS2-3Thi-BztA-Tyr-NH2 | 1944 |
| I-2479 | Ac-PL3-Asp-Npg-B5-Asn-Thr-Aib-Ala-Phe-Asn-PyrS2-3Thi-BztA-Leu-NH2 | 1945 |
| I-2480 | Ac-PL3-Asp-Npg-B5-Asn-Thr-Aib-Ala-Phe-Asn-PyrS2-3Thi-BztA-Val-NH2 | 1946 |
| I-2481 | Ac-PL3-Asp-Npg-B5-Asn-Thr-Aib-Ala-Phe-Asn-PyrS2-3Thi-BztA-Trp-NH2 | 1947 |
| I-2482 | Ac-PL3-Asp-Npg-B5-Asp-[CH2CCpCO2H]TriAzDap-Ala-Ala-Phe-Leu-PyrS2-3Thi- | 1948 |
| | BztA-Gln-NH2 | |
| I-2483 | Ac-PL3-Asp-Npg-B5-Asp-[CH2CChCO2H]TriAzDap-Ala-Ala-Phe-Leu-PyrS2-3Thi- | 1949 |
| | BztA-Gln-NH2 | |
| I-2484 | Ac-PL3-Asp-Npg-B5-Asp-3COOHF-Ala-Ala-Phe-Leu-PyrS2-2F3MeF-2F3MeW-Gln- | 1950 |
| | NH2 | |
| I-2485 | Ac-PL3-Asp-Npg-B5-Asp-3COOHF-Gln-Ala-Phe-Leu-PyrS2-2F3MeF-2F3MeW-Ala- | 1951 |
| | NH2 | |
| I-2486 | Ac-PL3-Asp-Npg-B5-Asp-3COOHF-Ala-Ala-Phe-Leu-PyrS2-2F3MeF-7C1BztA-Gln- | 1952 |
| | NH2 | |
| I-2487 | Ac-PL3-Asp-Npg-B5-Asp-3COOHF-Gln-Ala-Phe-Leu-PyrS2-2F3MeF-7C1BztA-Ala- | 1953 |
| | NH2 | |
| I-2488 | Ac-PL3-Asp-Npg-B5-Asp-3COOHF-Ala-Ala-Phe-Leu-PyrS2-2F3MeF-7FBztA-Gln- | 1954 |
| | NH2 | |
| I-2489 | Ac-PL3-Asp-Npg-B5-Asp-3COOHF-Gln-Ala-Phe-Leu-PyrS2-2F3MeF-7FBztA-Ala- | 1955 |
| | NH2 | |
| I-2490 | Ac-PL3-Asp-Npg-B5-Asp-3COOHF-Ala-Ala-Phe-Leu-PyrS2-2F3MeF-4C1BztA-Gln- | 1956 |
| | NH2 | |
| I-2491 | Ac-PL3-Asp-Npg-B5-Asp-3COOHF-Gln-Ala-Phe-Leu-PyrS2-2F3MeF-4C1BztA-Ala- | 1957 |
| | NH2 | |
| I-2492 | Ac-PL3-Asp-Npg-B5-Asn-Thr-Cpg-Ala-Phe-Asn-PyrS2-3Thi-BztA-Gln-NH2 | 1958 |
| I-2493 | Ac-PL3-Asp-Npg-B5-Asn-Thr-CyLeu-Ala-Phe-Asn-PyrS2-3Thi-BztA-Gln-NH2 | 1959 |
| I-2494 | Ac-PL3-Asp-Npg-B5-Asn-Thr-Aib-Ala-Phe-Asn-PyrS2-3Thi-BztA-Me2Asn-NH2 | 1960 |
| I-2495 | Ac-PL3-Asp-Npg-B5-Asn-Thr-Aib-Ala-Phe-Asn-PyrS2-3Thi-BztA-MeAsn-NH2 | 1961 |
| I-2496 | Ac-PL3-Asp-Npg-B5-Asn-Thr-Aib-Ala-Phe-Me2Asn-PyrS2-3Thi-BztA-Asn-NH2 | 1962 |
| I-2497 | Ac-PL3-Asp-Npg-B5-Asn-Thr-Aib-Ala-Phe-MeAsn-PyrS2-3Thi-BztA-Asn-NH2 | 1963 |
| I-2498 | Ac-PL3-Asn-Cha-B5-Asp-Gln-Hse-Ala-Phe-Leu-PyrS2-Phe-BztA-MorphAsn-NH2 | 1964 |
| I-2499 | Ac-PL3-Asn-Cha-B5-Asp-Gln-nLeu-Ala-Phe-Leu-PyrS2-Phe-BztA-MorphAsn-NH2 | 1965 |
| I-2500 | Ac-PL3-Asn-Cha-B5-Asp-Gln-hhLeu-Ala-Phe-Leu-PyrS2-Phe-BztA-MorphAsn-NH2 | 1966 |
| I-2501 | Ac-PL3-Asn-Cha-B5-Asp-Gln-Me2Gln-Ala-Phe-Leu-PyrS2-Phe-BztA-MorphAsn-NH2 | 1967 |
| I-2502 | Ac-PL3-Asn-Cha-B5-Asp-Gln-MeGln-Ala-Phe-Leu-PyrS2-Phe-BztA-MorphAsn-NH2 | 1968 |
| I-2503 | Ac-PL3-Asn-Cha-B5-Asp-Gln-Thr-Ala-Phe-Leu-PyrS2-Phe-BztA-MorphAsn-NH2 | 1969 |
| I-2504 | Ac-PL3-Asn-Cha-B5-Asp-Gln-Gln-Ala-Phe-Leu-PyrS2-Phe-BztA-MorphAsn-NH2 | 1970 |
| I-2505 | Ac-PL3-Asp-Npg-B6-Asp-3COOHF-Ala-Ala-Phe-Leu-PyrS-2F3MeF-BztA-Gln-NH2 | 1971 |
| I-2506 | Ac-PL3-Asp-Npg-B6-Asp-3COOHF-Gln-Ala-Phe-Leu-PyrS-2F3MeF-BztA-Ala-NH2 | 1972 |
| I-2507 | Ac-PL3-Asp-Npg-B6-Asp-3COOHF-Gln-Ala-Phe-Leu-PyrS-2F3MeF-BztA-Ala-NH2 | 1972 |
| I-2508 | Ac-PL3-Asp-Npg-B6-Asp-3COOHF-Ala-Ala-Phe-Leu-PyrS1-2F3MeF-BztA-Gln-NH2 | 1973 |
| I-2509 | Ac-PL3-Asp-Npg-B6-Asp-3COOHF-Ala-Ala-Phe-Leu-PyrS1-2F3MeF-BztA-Gln-NH2 | 1973 |
| I-2510 | Ac-PL3-Asp-Npg-B6-Asp-3COOHF-Gln-Ala-Phe-Leu-PyrS1-2F3MeF-BztA-Ala-NH2 | 1974 |
| I-2511 | Ac-PL3-Asp-Npg-B6-Asp-3COOHF-Ala-Ala-Phe-Leu-PyrS2-2F3MeF-BztA-Gln-NH2 | 1975 |
| I-2512 | Ac-PL3-Asp-Npg-B6-Asp-3COOHF-Ala-Ala-Phe-Leu-PyrS2-2F3MeF-BztA-Gln-NH2 | 1975 |
| I-2513 | Ac-PL3-Asp-Npg-B6-Asp-3COOHF-Ala-Ala-Phe-Leu-PyrS2-2F3MeF-BztA-Gln-NH2 | 1975 |
| I-2514 | Ac-PL3-Asp-Npg-B6-Asp-3COOHF-Ala-Ala-Phe-Leu-PyrS2-2F3MeF-BztA-Gln-NH2 | 1975 |
| I-2515 | Ac-PL3-Asp-Npg-B6-Asp-3COOHF-Gln-Ala-Phe-Leu-PyrS2-2F3MeF-BztA-Ala-NH2 | 1976 |
| I-2516 | Ac-PL3-Asp-Npg-B6-Asp-3COOHF-Gln-Ala-Phe-Leu-PyrS2-2F3MeF-BztA-Ala-NH2 | 1976 |
| I-2517 | Ac-PL3-Asp-Npg-B6-Asp-3COOHF-Gln-Ala-Phe-Leu-PyrS2-2F3MeF-BztA-Ala-NH2 | 1976 |
| I-2518 | Ac-PL3-Asp-Npg-B6-Asp-3COOHF-Ala-Ala-Phe-Leu-PyrS-2F3MeF-BztA-Gln-NH2 | 1971 |
| I-2519 | Ac-PL3-Asp-Npg-B6-Asp-3COOHF-Gln-Ala-Phe-Leu-PyrS1-2F3MeF-BztA-Ala-NH2 | 1974 |
| I-2520 | Ac-PL3-Asp-Npg-B5-Asp-[CH2CChCO2H]TriAzDap-Gln-Ala-Phe-Leu-PyrS2- | 1977 |
| | 2F3MeF-BztA-Ala-NH2 | |
| I-2521 | Ac-PL3-Asp-Npg-B5-Asp-[CH2CChCO2H]TriAzDap-Ala-Ala-Phe-Leu-PyrS2- | 1978 |
| | 2F3MeF-BztA-Ser-NH2 | |
| I-2522 | Ac-PL3-Asn-Cha-B5-Asp-Gln-[Ac]Lys-Ala-Phe-Leu-PyrS2-Phe-BztA-MorphAsn-NH2 | 1979 |
| I-2523 | Ac-PL3-Asn-Cha-B5-Asp-Gln-[Phc]Lys-Ala-Phe-Leu-PyrS2-Phe-BztA-MorphAsn- | 1980 |
| | NH2 | |
| I-2524 | Ac-PL3-Asn-Cha-B5-Asp-Gln-[MeSO2]Lys-Ala-Phe-Leu-PyrS2-Phe-BztA-MorphAsn- | 1981 |
| | NH2 | |
| I-2525 | Ac-PL3-Asn-Cha-B5-Asp-Gln-[CF3CO]Lys-Ala-Phe-Leu-PyrS2-Phe-BztA-MorphAsn- | 1982 |
| | NH2 | |
| I-2526 | Ac-PL3-Asn-Cha-B5-Asp-Gln-[mPEG4]Lys-Ala-Phe-Leu-PyrS2-Phe-BztA-MorphAsn- | 1983 |
| | NH2 | |
| I-2527 | Ac-PL3-Asn-Cha-B5-Asp-Gln-[MorphAc]Lys-Ala-Phe-Leu-PyrS2-Phe-BztA- | 1984 |
| | MorphAsn-NH2 | |
| I-2528 | Ac-PL3-Asn-Cha-B5-Asp-Gln-[Me2Npr]Lys-Ala-Phe-Leu-PyrS2-Phe-BztA- | 1985 |
| | MorphAsn-NH2 | |
| I-2529 | 5hexenyl-PL3-Asp-AllylGly-B5-Asp-3COOHF-Ala-Ala-Phe-Leu-PyrS2-2F3MeF- | 1986 |
| | BztA-Gln-NH2 | |
| I-2530 | 4pentenyl-PL3-Asp-AllylGly-B5-Asp-3COOHF-Ala-Ala-Phe-Leu-PyrS2-2F3MeF- | 1987 |
| | BztA-Gln-NH2 | |
| I-2531 | Ac-PL3-Asp-nLeu-B5-Asp-3COOHF-Ala-Ala-Phe-Leu-PyrS2-2F3MeF-BztA-Gln-NH2 | 1988 |
| I-2532 | Ac-PL3-Asp-Npg-B5-Asp-3COOHF-Ala-Ala-Phe-Leu-PyrS2-2F3MeF-BztA-Me2Gln- | 1989 |
| | NH2 | |
| I-2533 | Ac-PL3-Asp-Npg-B5-Asp-3COOHF-Ala-Ala-Phe-nLeu-PyrS2-2F3MeF-BztA-Me2Gln- | 1990 |
| | NH2 | |
| I-2534 | Ac-PL3-Asp-Npg-B5-Asp-3COOHF-Ala-Ala-Phe-Phe-PyrS2-2F3MeF-BztA-Me2Gln- | 1991 |
| | NH2 | |
| I-2535 | Ac-PL3-Asp-Npg-B5-Asp-3COOHF-Ala-Ala-Phe-Me2Gln-PyrS2-2F3MeF-BztA- | 1992 |
| | Me2Gln-NH2 | |
| I-2536 | Ac-PL3-Asp-Npg-B5-Asp-3COOHF-Ala-Ala-Phe-Me2Gln-PyrS2-2F3MeF-BztA- | 1992 |
| | Me2Gln-NH2 | |
| I-2537 | Ac-PL3-Asp-Npg-B5-Asp-3COOHF-Ala-Ala-Phe-MorphGln-PyrS2-2F3MeF-BztA- | 1993 |
| | Me2Gln-NH2 | |
| I-2538 | Ac-PL3-Asp-Npg-B5-Asp-3COOHF-Ala-Ala-Phe-MorphGln-PyrS2-2F3MeF-BztA- | 1993 |
| | Me2Gln-NH2 | |
| I-2539 | Ac-PL3-Asp-Npg-B5-Asp-3COOHF-Ala-Ala-Phe-AcLys-PyrS2-2F3MeF-BztA- | 1994 |
| | Me2Gln-NH2 | |
| I-2540 | Ac-PL3-Asp-Npg-B5-Asp-3COOHF-Ala-Ala-Phe-AcLys-PyrS2-2F3MeF-BztA- | 1994 |
| | Me2Gln-NH2 | |
| I-2541 | Ac-PL3-Asp-Npg-B5-Asp-3COOHF-Ala-Ala-Phe-Leu-PyrS2-2F3MeF-BztA- | 1995 |
| | MorphGln-NH2 | |
| I-2542 | Ac-PL3-Asp-Npg-B5-Asp-3COOHF-Ala-Ala-Phe-nLeu-PyrS2-2F3MeF-BztA- | 1996 |
| | MorphGln-NH2 | |
| I-2543 | Ac-PL3-Asp-Npg-B5-Asp-3COOHF-Ala-Ala-Phe-Phe-PyrS2-2F3MeF-BztA- | 1997 |
| | MorphGln-NH2 | |
| I-2544 | Ac-PL3-Asp-Npg-B5-Asp-3COOHF-Ala-Ala-Phe-Me2Gln-PyrS2-2F3MeF-BztA- | 1998 |
| | MorphGln-NH2 | |
| I-2545 | Ac-PL3-Asp-Npg-B5-Asp-3COOHF-Ala-Ala-Phe-Me2Gln-PyrS2-2F3MeF-BztA- | 1998 |
| | MorphGln-NH2 | |
| I-2546 | Ac-PL3-Asp-Npg-B5-Asp-3COOHF-Ala-Ala-Phe-MorphGln-PyrS2-2F3MeF-BztA- | 1999 |
| | MorphGln-NH2 | |
| I-2547 | Ac-PL3-Asp-Npg-B5-Asp-3COOHF-Ala-Ala-Phe-MorphGln-PyrS2-2F3MeF-BztA- | 1999 |
| | MorphGln-NH2 | |
| I-2548 | Ac-PL3-Asp-Npg-B5-Asp-3COOHF-Ala-Ala-Phe-AcLys-PyrS2-2F3MeF-BztA- | 2000 |
| | MorphGln-NH2 | |
| I-2549 | Ac-PL3-Asp-Npg-B5-Asp-3COOHF-Ala-Ala-Phe-AcLys-PyrS2-2F3MeF-BztA- | 2000 |
| | MorphGln-NH2 | |
| I-2550 | Bn3OAllyl-AcAsp-Npg-B5-Asp-TfeGA-Ala-Ala-Phe-Leu-PyrS2-3Thi-BztA-Gln-NH2 | 2001 |
| I-2551 | Ac-PL3-Asp-Npg-B5-Asp-3COOHF-Aib-Ala-Phe-nLeu-PyrS2-3Thi-BztA- | 2002 |
| | [NMe2]GlnR-NH2 | |
| I-2552 | Ac-PL3-Asp-Npg-B5-Asp-3COOHF-Aib-Ala-Phe-nLeu-PyrS2-3Thi-BztA- | 2003 |
| | NHEt]GlnR-NH2 | |
| I-2553 | Ac-PL3-Asp-Npg-B5-Asp-3COOHF-Aib-Ala-Phe-nLeu-PyrS2-3Thi-BztA- | 2004 |
| | [Morph]GlnR-NH2 | |
| I-2554 | Ac-PL3-Asp-Npg-B5-Asp-3COOHF-Aib-Ala-Phe-nLeu-PyrS2-3Thi-BztA- | 2005 |
| | [bismethoxyethylamine]GlnR-NH2 | |
| I-2555 | Ac-PL3-Asp-Npg-B5-Asp-3COOHF-Aib-Ala-Phe-nLeu-PyrS2-3Thi-BztA- | 2006 |
| | [4Pippip]GlnR-NH2 | |
| I-2556 | Ac-PL3-Asp-Npg-B5-Asp-3COOHF-Aib-Ala-Phe-nLeu-PyrS2-3Thi-BztA- | 2007 |
| | [AcPpz]GlnR-NH2 | |
| I-2557 | Ac-PL3-Asp-Npg-B5-Asp-3COOHF-Aib-Ala-Phe-nLeu-PyrS2-3Thi-BztA- | 2008 |
| | 4F3CPip]GlnR-NH2 | |
| I-2558 | Ac-PL3-Asp-Npg-B5-Asp-3COOHF-Aib-Ala-Phe-Abu-PyrS2-3Thi-BztA- | 2009 |
| | [Morph]GlnR-NH2 | |
| I-2559 | Ac-PL3-Asp-Npg-B5-Asp-3COOHF-Aib-Ala-Phe-Abu-PyrS2-3Thi-BztA- | 2010 |
| | [bismethoxyethylamine]GlnR-NH2 | |
| I-2560 | Ac-PL3-Asp-Npg-B5-Asp-3COOHF-Aib-Ala-Phe-Abu-PyrS2-3Thi-BztA- | 2011 |
| | [4Pippip]GlnR-NH2 | |
| I-2561 | Ac-PL3-Asp-Npg-B5-Asp-3COOHF-Aib-Ala-Phe-Abu-PyrS2-3Thi-BztA- | 2012 |
| | [AcPpz]GlnR-NH2 | |
| I-2562 | Ac-PL3-Tyr-TfeGA-B5-Ala-Asp-Asp-Ala-Ala-4BrF-S8-Ala-Phe-BztA-Gln-NH2 | 2013 |
| I-2563 | Ac-R3-Asp-Npg-B5-Asp-3COOHF-Ala-Ala-Phe-Leu-PyrS2-2F3MeF-BztA-Gln-NH2 | 2014 |
| I-2564 | Ac-R5-Asp-Npg-B5-Asp-3COOHF-Ala-Ala-Phe-Leu-PyrS2-2F3MeF-BztA-Gln-NH2 | 2015 |
| I-2565 | Ac-R5-Asp-Npg-B5-Asp-3COOHF-Ala-Ala-Phe-Leu-PyrS2-2F3MeF-BztA-Gln-NH2 | 2015 |
| I-2566 | Ac-Pro-R3-Asp-Npg-B5-Asp-3COOHF-Ala-Ala-Phe-Leu-PyrS2-2F3MeF-BztA-Gln- | 2016 |
| | NH2 | |
| I-2567 | Ac-Pro-R5-Asp-Npg-B5-Asp-3COOHF-Ala-Ala-Phe-Leu-PyrS2-2F3MeF-BztA-Gln- | 2017 |
| | NH2 | |
| I-2568 | Ac-PyrR-Ala-Asp-Npg-B5-Asp-3COOHF-Ala-Ala-Phe-Leu-PyrS2-2F3MeF-BztA-Gln- | 2018 |
| | NH2 | |
| I-2569 | Ac-PyrR-Glu-Asp-Npg-B5-Asp-3COOHF-Ala-Ala-Phe-Leu-PyrS2-2F3MeF-BztA-Gln- | 2019 |
| | NH2 | |
| I-2570 | Ac-R3-Asp-Npg-B5-Asp-3COOHF-Ala-Ala-Phe-Leu-PyrS2-2F3MeF-BztA-Gln-NH2 | 2014 |
| I-2571 | Ac-R5-Asp-Npg-B5-Asp-3COOHF-Ala-Ala-Phe-Leu-PyrS2-2F3MeF-BztA-Gln-NH2 | 2015 |
| I-2572 | Ac-Pro-R3-Asp-Npg-B5-Asp-3COOHF-Ala-Ala-Phe-Leu-PyrS2-2F3MeF-BztA-Gln- | 2016 |
| | NH2 | |
| I-2573 | Ac-Pro-R5-Asp-Npg-B5-Asp-3COOHF-Ala-Ala-Phe-Leu-PyrS2-2F3MeF-BztA-Gln- | 2017 |
| | NH2 | |
| I-2574 | Ac-R5-Ala-Asp-Npg-B5-Asp-3COOHF-Ala-Ala-Phe-Leu-PyrS2-2F3MeF-BztA-Gln- | 2020 |
| | NH2 | |
| I-2575 | Ac-Pro-R5-Glu-Asp-Npg-B5-Asp-3COOHF-Ala-Ala-Phe-Leu-PyrS2-2F3MeF-BztA- | 2021 |
| | Gln-NH2 | |
| I-2576 | Ac-PL3-Asp-Npg-B5-Asp-3COOHF-Aib-Ala-Phe-dSer-PyrS2-3Thi-BztA-dSer-Ala- | 2022 |
| | NH2 | |
| I-2577 | Ac-PL3-Asp-Npg-B5-Asp-3COOHF-Aib-Ala-Phe-Ser-PyrS2-3Thi-BztA-Ser-Ala-NH2 | 2023 |
| I-2578 | Ac-PL3-Asp-Npg-B5-Asp-3COOHF-Aib-Ala-Phe-AcLys-PyrS2-3Thi-BztA-Ala-Ala- | 2024 |
| | NH2 | |
| I-2579 | Ac-PL3-Asp-Npg-B5-Asp-3COOHF-Aib-Ala-Phe-AcLys-PyrS2-2F3MeF-BztA-Ala- | 2025 |
| | Ala-NH2 | |
| I-2580 | Ac-PL3-Asp-Npg-B5-Asp-3COOHF-Ala-Ala-Phe-Leu-SPip1-2F3MeF-BztA-Gln-NH2 | 2026 |
| I-2581 | Ac-PL3-Asp-Npg-B5-Asp-3COOHF-Ala-Ala-Phe-Leu-SPip2-2F3MeF-BztA-Gln-NH2 | 2027 |
| I-2582 | Ac-PL3-Asp-Npg-B5-Asp-3COOHF-Ala-Ala-Phe-Leu-SPip2-2F3MeF-BztA-Gln-NH2 | 2027 |
| I-2583 | Ac-PL3-Asp-Npg-B5-Asp-3COOHF-Ala-Ala-Phe-Leu-SPip3-2F3MeF-BztA-Gln-NH2 | 2028 |
| I-2584 | Ac-PL3-Asp-Npg-B5-Asp-3COOHF-Gln-Ala-Phe-Leu-SPip1-2F3MeF-BztA-Ala-NH2 | 2029 |
| I-2585 | Ac-PL3-Asp-Npg-B5-Asp-3COOHF-Gln-Ala-Phe-Leu-SPip2-2F3MeF-BztA-Ala-NH2 | 2030 |
| I-2586 | Ac-PL3-Asp-Npg-B5-Asp-3COOHF-Gln-Ala-Phe-Leu-SPip2-2F3MeF-BztA-Ala-NH2 | 2030 |
| I-2587 | Ac-PL3-Asp-Npg-B5-Asp-3COOHF-Gln-Ala-Phe-Leu-SPip3-2F3MeF-BztA-Ala-NH2 | 2031 |
| I-2588 | Ac-PL3-Asp-Npg-B5-Asp-3COOHF-Ala-Ala-Phe-Leu-SPip2-3Thi-BztA-Gln-NH2 | 2032 |
| I-2589 | Ac-PL3-Asp-Npg-B5-Asp-3COOHF-Ala-Ala-Phe-Leu-SPip2-3Thi-BztA-Gln-NH2 | 2032 |
| I-2590 | Ac-PL3-Asp-Npg-B5-Asp-3COOHF-Ala-Ala-Phe-Leu-SPip3-3Thi-BztA-Gln-NH2 | 2033 |
| I-2591 | Ac-PL3-Asp-Npg-B5-Asp-3COOHF-Gln-Ala-Phe-Leu-SPip1-3Thi-BztA-Ala-NH2 | 2034 |
| I-2592 | Ac-PL3-Asp-Npg-B5-Asp-3COOHF-Gln-Ala-Phe-Leu-SPip2-3Thi-BztA-Ala-NH2 | 2035 |
| I-2593 | Ac-PL3-Asp-Npg-B5-Asp-3COOHF-Gln-Ala-Phe-Leu-SPip2-3Thi-BztA-Ala-NH2 | 2035 |
| I-2594 | Ac-PL3-Asp-Npg-B5-Asp-TfeGA-Ala-Ala-Phe-Leu-PyrS2-3Thi-BztA-Gln-NH2 | 571 |
| I-2595 | Ac-PL3-Asp-Npg-B5-Asp-TfeGA-Ala-Ala-Phe-Leu-PyrS2-3Thi-BztA-Gln-NH2 | 571 |
| I-2596 | Ac-PL3-Asp-Npg-B5-Asp-3COOHF-Ala-Ala-Phe-Leu-PyrS2-2F3MeF-BztA-Gln-NH2 | 1116 |
| I-2597 | Ac-PL3-Asp-Npg-B5-Asp-3COOHF-Ala-Ala-Phe-Leu-PyrS2-2F3MeF-BztA-Gln-NH2 | 1116 |
| I-2598 | 5hexenyl-MePro-Asp-B5-Ala-Asp-Asp-Ala-Ala-Phe-PyrS2-Leu-3Thi-BztA-Gln-NH2 | 2036 |
| I-2599 | 4pentenyl-MePro-Asp-B5-Ala-Asp-Asp-Ala-Ala-Phe-PyrS2-Leu-3Thi-BztA-Gln-NH2 | 2037 |
| I-2600 | 5hexenyl-MePro-Asp-B5-Ala-Asp-Asp-Ala-Ala-Phe-PyrS2-Leu-3Thi-BztA-Gln-NH2 | 2036 |
| I-2601 | 4pentenyl-MePro-Asp-B5-Ala-Asp-Asp-Ala-Ala-Phe-PyrS2-Leu-3Thi-BztA-Gln-NH2 | 2037 |
| I-2602 | 5hexenyl-MePro-Asp-B5-Ala-Asp-3COOHF-Ala-Ala-Phe-PyrS2-Leu-3Thi-BztA-Gln- | 2038 |
| | NH2 | |
| I-2603 | 4pentenyl-MePro-Asp-B5-Ala-Asp-3COOHF-Ala-Ala-Phe-PyrS2-Leu-3Thi-BztA-Gln- | 2039 |
| | NH2 | |
| I-2604 | 5hexenyl-MePro-Asp-B5-Ala-Asp-3COOHF-Ala-Ala-Phe-PyrS2-Leu-3Thi-BztA-Gln- | 2038 |
| | NH2 | |
| I-2605 | 4pentenyl-MePro-Asp-B5-Ala-Asp-3COOHF-Ala-Ala-Phe-PyrS2-Leu-3Thi-BztA-Gln- | 2039 |
| | NH2 | |
| I-2606 | BzAm2Allyl-MePro-Asp-B5-Ala-Asp-Asp-Ala-Ala-Phe-PyrS2-Leu-3Thi-BztA-Gln- | 2040 |
| | NH2 | |
| I-2607 | BzAm2Allyl-MePro-Asp-B5-Ala-Asp-3COOHF-Ala-Ala-Phe-PyrS2-Leu-3Thi-BztA- | 2041 |
| | Gln-NH2 | |
| I-2608 | 3butenyl-MePro-Asp-B5-Ala-Asp-Asp-Ala-Ala-Phe-PyrS2-Leu-3Thi-BztA-Gln-NH2 | 2042 |
| I-2609 | 5hexenyl-MePro-Asp-B5-Ala-Asp-Asp-Ala-Ala-Phe-PyrS2-Leu-3Thi-BztA-Gln-NH2 | 2036 |
| I-2610 | Ac-PL3-Asp-Npg-B5-Asp-3COOHF-Ala-Ala-Phe-Leu-Az2-2F3MeF-BztA-Gln-NH2 | 2043 |
| I-2611 | Ac-PL3-Asp-Npg-B5-Asp-3COOHF-Ala-Ala-Phe-Leu-Az3-2F3MeF-BztA-Gln-NH2 | 2044 |
| I-2612 | Ac-PL3-Asp-Npg-B5-Asp-3COOHF-Ala-Ala-Phe-Leu-Az3-3Thi-BztA-Gln-NH2 | 2045 |
| I-2613 | Ac-PL3-Asp-Npg-B5-Asp-3COOHF-Aib-Ala-Phe-Leu-Az2-2F3MeF-BztA-Gln-NH2 | 2046 |
| I-2614 | Ac-PL3-Asp-Npg-B5-Asp-3COOHF-Aib-Ala-Phe-Leu-Az2-2F3MeF-BztA-Gln-NH2 | 2046 |
| I-2615 | Ac-PL3-Asp-Npg-B5-Asp-3COOHF-Aib-Ala-Phe-Leu-Az3-2F3MeF-BztA-Gln-NH2 | 2047 |
| I-2616 | Ac-PL3-Asp-Npg-B5-Asp-3COOHF-Aib-Ala-Phe-Leu-Az3-2F3MeF-BztA-Gln-NH2 | 2047 |
| I-2617 | Ac-PL3-Asp-Npg-B5-Asp-3COOHF-Aib-Ala-Phe-Leu-Az2-3Thi-BztA-Gln-NH2 | 2048 |
| I-2618 | Ac-PL3-Asp-Npg-B5-Asp-3COOHF-Aib-Ala-Phe-Leu-Az3-3Thi-BztA-Gln-NH2 | 2049 |
| I-2619 | Ac-PL3-Asp-Npg-B5-Asp-3COOHF-Aib-Ala-Phe-Leu-Az3-3Thi-BztA-Gln-NH2 | 2049 |

### Example 6. Synthesis of Compound 2-2

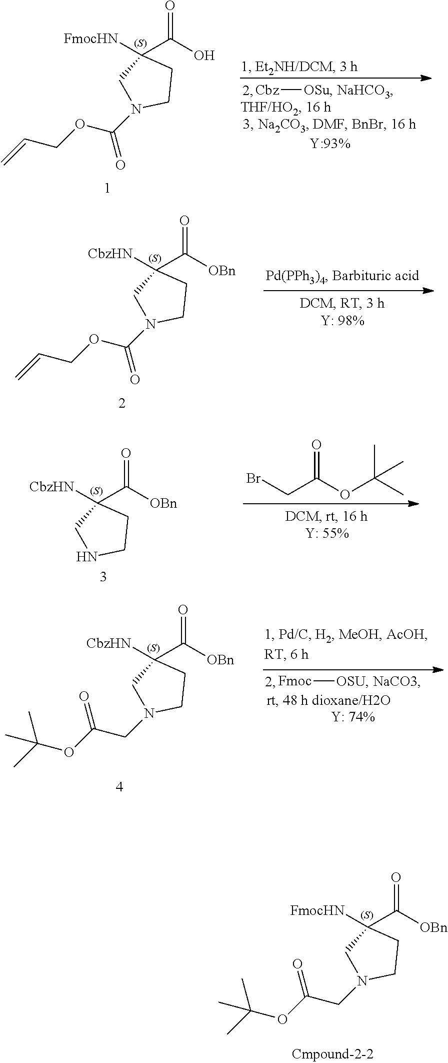

Step 1: 1-Allyl 3-benzyl (S)-3-(((benzyloxy)carbonyl)amino)pyrrolidine-1,3-dicarboxylate (2). A mixture of (S)-3-((((9H-fluoren-9-yl)methoxy)carbonyl)amino)-1-((allyloxy) carbonyl)pyrrolidine-3-carboxylic acid (20 g, 45.9 mmol) in DCM (300 mL) and Et₂N (300 mL) was stirred at room temperature for 3 h. The mixture was concentrated and dissolved into THF (400 mL) and water (400 mL). Cbz-OSU (17.1 g, 68.9 mmol) and NaHCO₃ (7.71 g, 91.7 mmol) was add. The reaction mixture was stirred at room temperature for 16 h. The mixture was adjusted pH to 3-4 with 1N HCl. The aqueous phase was extracted with EtOAc (3×800 mL). The desired EtOAc layer was then dried, concentrated to afford the crude product and dissolved into DMF (500 mL). BnBr (15.69 g, 91.7 mmol) and Na₂CO₃ (9.72 g, 91.7 mmol) was added and stirred at room temperature for 16 h. The reaction mixture was diluted with ethyl acetate (2 L), washed with brine (5×500 mL), dried over Na₂SO₄, concentrated and purified by silica gel column chromatography (eluted with hexane/ethyl acetate=2:1, V/V) to afford the product (18.7 g, 93% yield) as a brown oil. MS (ESI): m z=439.1 [M+H]⁺.

Step 2: Benzyl (S)-3-(((benzyloxy)carbonyl)amino)pyrrolidine-3-carboxylate (3). A mixture of 1-allyl 3-benzyl (S)-3-(((benzyloxy)carbonyl)amino)pyrrolidine -1,3-dicarboxylate (9.35 g, 21.3 mmol), Pd(PPh₃)₄ (4.93 g, 4.3 mmol) and Barbituric acid (5.46 g, 42.7 mmol) in DCM (300 mL) under Ar was stirred at room temperature for 3 h. The mixture was concentrated and purified by silica gel column chromatography (eluted with DCM/MeOH=10:1, V/V) to afford the product (7.5 g, 98% yield) as a brown oil. MS (ESI): m/z=355.1 [M+H]⁺.

Step 3: Benzyl (S)-3-(((benzyloxy)carbonyl)amino)-1-(2-(tert-butoxy)-2-oxoethyl) pyrrolidine-3-carboxylate (4). A mixture of benzyl (S)-3-(((benzyloxy)carbonyl)amino)pyrrolidine-3-carboxylate (15 g, 42.4 mmol) and tert-butyl 2-bromoacetate (16.5 g, 84.7 mmol) in DCM (400 mL) was stirred at room temperature for 16 h. Et₂NH (12.4 g, 169.5 mmol) was added and stirred at room temperature for 3 h. The mixture was adjusted PH to 8-9 with sat. NaHCO₃. The aqueous phase was extracted with DCM (3×500 mL). The desired DCM layers was then dried, concentrated purified by silica gel column chromatography (eluted with hexane/ethyl acetate=2:1, V/V) to afford the product (10.9 g, 55% yield) as a yellow oil. MS (ESI): m z=469.2 [M+H]⁺.

Step 4: (S)-3-((((9H-fluoren-9-yl)methoxy)carbonyl)amino)-1-(2-(tert-butoxy)-2-oxoethyl)pyrrolidine-3-carboxylic acid (compound-2-2). A mixture of benzyl (S)-3-(((benzyloxy)carbonyl)amino)-1-(2-(tert-butoxy)-2-oxoethyl)pyrrolidine-3-carboxylate (14.5 g, 31 mmol) and Palladium on carbon (6 g, 10%) in MeOH (600 mL) and AcOH (20 mL) was attached to a hydrogenation apparatus. The system was evacuated and then refilled with hydrogen. The mixture was stirred at room temperature for 6 h. The reaction mixture was filtered out and the filtrate was concentrated and dissolved into dioxane (300 mL) and water (300 mL). FmocOSu (20.88 g, 62 mmol) and NaHCO₃ (13 g, 155 mmol) was added. The mixture was stirred at room temperature for 48 h. The mixture was adjusted PH to 3-4 with 0.5 N HCl. The aqueous phase was extracted with DCM (3×500 mL). The desired DCM layers was then dried and concentrated. The resulting solid was recrystallized from methanol: EtOAc: PE=1:1:1 to give the product (10.62 g, 74% yield) as a white solid. MS (ESI): m/z=467.0 [M+H]⁺. 400 MHz, DMSO-d6, δ 7.90-7.89 (m, 3H); 7.73 (d, J=7.6 Hz, 2H); 7.42 (t, J=7.2 Hz, 2H); 7.34 (t, J=7.4 Hz, 2H); 4.30-4.20 (m, 3H); 3.27-3.18 (m, 2H); 3.13 (d, J=10 Hz, 1H); 2.93 (d, J=10 Hz, 1H); 2.84-2.79 (m, 1H); 2.66-2.60 (m, 1H); 2.24-2.17 (m, 1H); 2.06-2.00 (m, 1H); 1.41 (s, 9H). Purity by HPLC: 99.78% (214 nm), RT=16.29 min; Mobile Phase: A: Water (0.05% TFA) B: ACN (0.05% TFA); Gradient: 20% B for 1 min, increase to 80% B within 20 min, increase to 95% B within 1 min, hold for 5 min, back to 20% B within 0.1 min. Flow Rate: 1 mL/min; Column: XBridge Peptide BEH C18, 4.6*150 mm, 3.5 m. Column Temperature: 40° C. Purity by SFC: 99.83%, Column AD-H: RT 1.71 min; 100%, Column AS-H: RT 3.53 min; 100%, Column OD-H: RT 1.48 min; 99.70%, Column OJ-H: RT 2.42 min.

### Example 7. Synthesis of a Compound

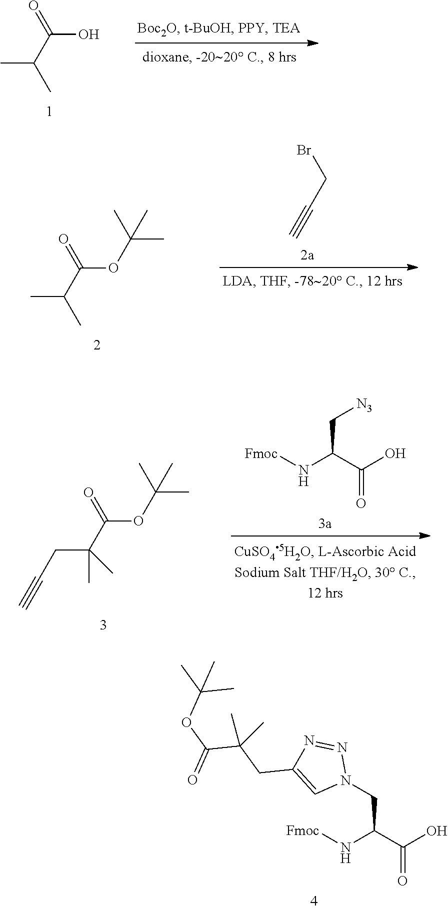

Preparation of compound 2. A mixture of compound 1 (30.0 g, 340 mmol, 31.5 mL, 1 eq), t-BuOH (27.7 g, 374 mmol, 35.8 mL, 1.1 eq), TEA (68.9 g, 681 mmol, 94.7 mL, 2 eq), and 4-pyrrolidin-1-ylpyridine (2.52 g, 17.0 mmol, 0.05 eq) in dioxane (20 mL) was stirred at −20° C. for 0.5 hr and then Boc₂O (96.6 g, 442 mmol, 101 mL, 1.3 eq) was added. The resulting mixture was stirred at 20° C. for 7.5 hrs. TLC (petroleum ether/ethyl acetate=10/1) showed starting material (R_{f}=0.1) was consumed completely. The mixture was diluted with DCM (100 mL), washed with 2N HCl (100 mL*2) and sat.aq.NaHCO₃ (100 mL), dried over anhydrous sodium sulfate and filtered. The filtrate was purified through distillation (62° C.) under reduced pressure (vacuum degree:-0.95 MPa) to give compound 2 (27.0 g, 187 mmol, 55.0% yield) as a colorless oil. ¹H NMR: 400 MHz CDCl3: δ=3.71 (s, 1H), 2.39-2.46 (m, 1H), 1.45 (s, 9H), 1.12 (d, J=8.0 Hz, 6H).

Preparation of compound 3. To a solution of i-Pr₂NH (28.2 g, 279 mmol, 39.4 mL, 1.15 eq) in THF (80.0 mL) was added drop-wise n-BuLi (2.5 M, 106 mL, 1.1 eq) at −78° C. and stirred for 1 hr. The fresh prepared LDA was added drop-wise to a solution of compound 2 (35.0 g, 242 mmol, 1 eq) in THF (80.0 mL) at 0° C. After addition, the reaction was stirred at 20° C. for 1 hr before cooling back to 0° C. A solution of compound 2a (39.7 g, 266 mmol, 28.7 mL, 80% purity, 1.1 eq) in THF (20.0 mL) was added drop-wise. The resulting mixture was stirred at 20° C. for 10 hrs. TLC (petroleum ether/ethyl acetate=5/1) showed new spot (R_{f}=0.65) formed. The mixture was quenched with water (150 mL), the organic phase was separated and the aqueous layer extracted with MTBE (3×60.0 mL). The combined organic layers were washed with a saturated aqueous NaCl solution, dried over sodium sulphate, filtered and concentrated in vacuo. The obtained crude oil was distilled (94° C., —0.95Mpa) to give compound 3 (27.0 g, 148 mmol, 61.0% yield) as a colorless oil. ¹H NMR: 400 MHz CDCl₃: δ=2.39 (d, J=8.0 Hz, 2H), 1.99 (t, J=4.0 Hz, 1H), 1.45 (s, 9H), 1.24 (s, 6H).

Preparation of compound 4. To a solution of compound 3 (16.7 g, 119 mmol, 1.2 eq) in THF (200 mL) was added a solution of CuSO₄·5H₂O (868 mg, 3.48 mmol, 0.035 eq) and L-Ascorbic Acid Sodium Salt (5.12 g, 25.8 mmol, 0.26 eq) in H₂O (100 mL) followed by addition of a solution of compound 3a (35 g, 99.33 mmol, 1 eq) in THF (200 mL) and H₂O (200 mL). The resulting mixture was stirred at 30° C. for 12 hrs. LCMS showed desired MS (Rt=0.977 min) was detected. The mixture was concentrated under vacuum to remove THF and white solids were precipitated out, filtered. The solid was triturated with MeOH/H₂O (1/1, 2 L) to give compound 4 (22.0 g, 39.7 mmol, 40.0% yield, 96.5% purity) as a white solid. In one LCMS run: Rt=0.977 min, m/z: [M+H]⁺=535.4. In another LCMS run: Rt=0.956 min, m/z: [M+H]⁺=535.3. In a HPLC run: Rt=2.601 min. ¹H NMR: 400 MHz DMSO-d6: δ=7.88 (d, J=8.0 Hz, 2H), 7.63-7.71 (m, 4H), 7.41 (t, J=8.0 Hz, 2H), 7.32 (t, J=8.0 Hz, 2H), 4.75 (s, 1H), 4.33-4.68 (m, 2H), 4.10-4.29 (m, 3H), 2.75 (s, 2H), 1.36 (s, 9H), 1.02 (d, J=2.4 Hz, 1H).

### Example 8. Synthesis of a Compound

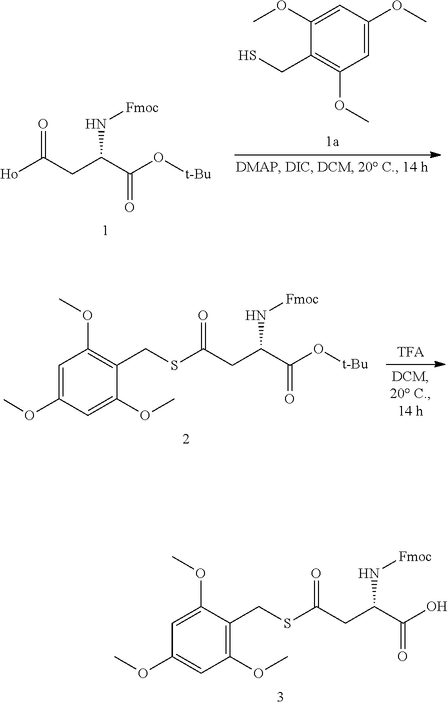

To a solution of compound 1a (5.00 g, 23.3 mmol, 1.00 eq), compound 1 (14.4 g, 35.0 mmol, 1.50 eq) and DMAP (142 mg, 1.17 mmol, 0.050 eq) in dichloromethane (80.0 mL) was added DIC (3.59 g, 28.4 mmol, 4.41 mL, 1.22 eq) under a nitrogen atmosphere. The reaction was stirred at 20° C. for 14 hrs. LCMS showed the starting material was consumed completely with desired mass (in one run, Rt=1.080 min) was detected. TLC (Petroleum ether:Ethyl acetate=3:1) also showed the starting material (Rf=0.89) was consumed completely with six new spots (R_{f}=0.37) formed. The mixture was filtered and the precipitate was washed with dichloromethane (10.0 mL*3). The filtrate was concentrated under reduced pressure. The residue was purified by column chromatography (SiO₂, Petroleum ether:Ethyl acetate=50:1 to 2:1, R_{f}=0.37). The compound 2 (14.9 g, crude) was obtained as yellow oil. LCMS: product: Rt=1.080 min, m/z=630 (M+H)+. ¹HNMR: CDCl₃, 400 MHz: δ: 7.71 (d, J=7.6 Hz, 2H), 7.62 (d, J=7.6 Hz, 2H), 7.41 (t, J=7.6 Hz, 2H), 7.32 (td, J=7.6 Hz, J₂=0.8 Hz, 2H), 6.10 (s, 2H), 5.80 (d, J=8.4 Hz, 1H), 4.55-4.51 (m, 1H), 4.42-4.36 (m, 2H), 4.30-4.22 (m, 3H), 3.80-3.79 (m, 9H), 3.24-3.04 (m, 2H), 1.49 (s, 9H).

To a solution of compound 2 (12.9 g, 21.2 mmol, 1.00 eq) in dichloromethane (45.0 mL) was added TFA (23.1 g, 202 mmol, 15.0 mL, 9.54 eq), the reaction was stirred at 20° C. for 14 hrs. LCMS showed the starting material was consumed completely with desired mass (Rt=0.828 min, m/z=550) was detected. The volatiles (Dichloromethane, TFA) was removed under reduced pressure. The crude product was purified by reversed-phase HPLC (FA). Compound 3 (5.52 g, 9.64 mmol, 45.4% yield, 96.3% purity) was obtained as light yellow solid. In a LCMS run: product: Rt=0.828 min, m/z=550.2 (M−H)⁻. In another LCMS run: product: Rₜ=0.835 min, m/z=550.2 (M−H)⁻. HPLC: product: Rₜ=3.566 min, purity: 96.3%. ¹HNMR: CDCl₃, 400 MHz: δ: 7.77 (d, J=7.6 Hz, 2H), 7.61 (d, J=7.2 Hz, 2H), 7.41 (t, J=7.6 Hz, 2H), 7.32 (td, J₁=7.6 Hz, J₂=0.8 Hz, 2H), 6.10 (s, 2H), 5.82 (d, J=8.0 Hz, 1H), 4.70-4.67 (m, 1H), 4.44-4.39 (m, 2H), 4.28-4.26 (m, 2H), 4.25-4.23 (m, 1H), 3.80 (s, 9H), 3.35-3.31 (m, 2H).

### Example 9. Synthesis of a Compound

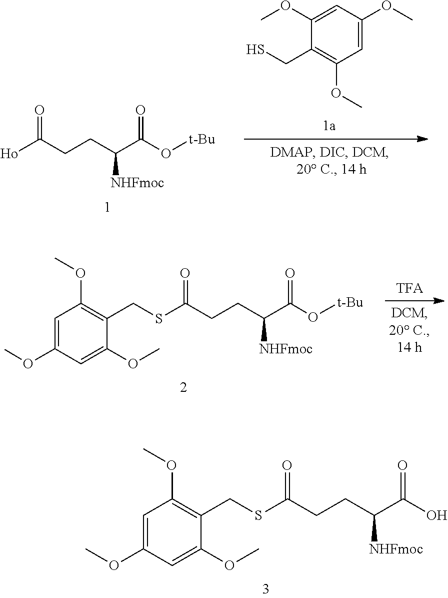

To a solution of compound 1a (5.00 g, 23.3 mmol, 1.00 eq), compound 1 (14.8 g, 35.0 mmol, 1.50 eq) and DMAP (142 mg, 1.17 mmol, 0.050 eq) in Dichloromethane (80.0 mL) was added DIC (3.59 g, 28.4 mmol, 4.41 mL, 1.22 eq) under a nitrogen atmosphere. The reaction was stirred at 20° C. for 14 hrs. LCMS showed the starting material was consumed completely with desired mass (Rt=1.081 min) was detected. TLC (Petroleum ether:Ethyl acetate=3:1) also showed the starting material (Rf=0.88) was consumed completely with six new spots (Rf=0.30) formed. The mixture was filtered and the precipitate was washed with Dichloromethane (10.0 mL*3). The filtrate was concentrated under reduced pressure. The residue was purified by column chromatography (SiO₂, Petroleum ether:Ethyl acetate=50:1 to 2:1, Rf=0.30). The compound 2 (15.8 g, crude) was obtained as colorless oil. LCMS: product: Rt=1.081 min, m/z=644 (M+H)+. ¹HNMR: CDCl₃, 400 MHz δ: 7.77 (d, J=7.2 Hz, 2H), 7.61 (d, J=7.6 Hz, 2H), 7.40 (t, J=7.6 Hz, 2H), 7.32 (tt, J₁=7.6 Hz, J₂=1.2 Hz, 2H), 6.10 (s, 2H), 5.39 (d, J=8.0 Hz, 1H), 4.47-4.34 (m, 2H), 4.31-4.25 (m, 1H), 4.24-4.20 (m, 3H), 3.80 (s, 9H), 2.67-2.53 (m, 2H), 2.28-0.20 (m, 1H), 2.03-1.99 (m, 1H), 1.48 (s, 9H).

To a solution of compound 2 (15.8 g, 25.4 mmol, 1.00 eq) in dichloromethane (45.0 mL) was added TFA (23.1 g, 202 mmol, 15.0 mL, 7.97 eq), the reaction was stirred at 20° C. for 14 hrs. LCMS showed the starting material was consumed completely with desired mass (Rt=0.829 min, m/z=564) was detected. The volatiles (Dichloromethane, TFA) was removed under reduced pressure. The crude product was purified by reversed-phase HPLC (FA). Compound 3 (6.57 g, 11.4 mmol, 45.1% yield, 98.7% purity) was obtained as white solid. In a LCMS run: product: Rt=0.829 min, m/z=564.3 (M−H)⁻. In another LCMS run: product: Rt=0.838 min, m/z=564.3 (M−H)⁻. In a HPLC run: product: Rt=3.593 min, purity: 98.7%. ¹HNMR: CDCl₃, 400 MHz: δ: 7.76 (d, J=7.6 Hz, 2H), 7.60 (d, J=7.2 Hz, 2H), 7.40 (t, J=7.6 Hz, 2H), 7.32 (t, J=7.6 Hz, 2H), 6.10 (s, 2H), 5.53 (d, J=8.0 Hz, 1H), 4.53-4.46 (m, 3H), 4.27-4.21 (m, 3H), 3.79 (s, 9H), 2.77-2.53 (m, 2H), 2.33-2.28, m, 1H), 2.16-2.04, m, 1H).

In some embodiments, it was confirmed that various peptides, e.g., stapled peptides, comprising residues of amino acids described herein can provide higher affinity than reference peptides that comprise a reference amino acid, e.g., a natural amino acid such as Asp or Glu, but are otherwise identical.

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described in the present disclosure. It is, therefore, to be understood that the foregoing embodiments are presented by way of example only and that, provided technologies, including those to be claimed, may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. An agent, wherein the agent is or comprise a peptide comprising:
X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,
wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic group;
X⁵ comprises a side chain comprising an acidic group;
X⁶ comprises a side chain comprising an acidic group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴ are stapled and X⁴ and X¹¹ are stapled.

2. An agent, wherein the agent is or comprise a peptide comprising:
X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,
wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group; and
X¹³ comprises a side chain comprising an optionally substituted aromatic group.

3. An agent, wherein the agent is or comprises:
X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]p¹⁸[X¹⁹]ₚ₁₉[X²⁰]ₚ₂₀[X²¹]ₚ₂₁[X²²]ₚ₂₂[X²³]ₚ₂₃,
wherein: each of p14, p15, p16, p17, p18, p19, p20, p21, p22, and p23 is independently 0 or 1, and each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue.

4. An agent, wherein the agent is or comprises:
[X]pX¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X]p′;
wherein:
each of p14, p15, p¹⁶ and p17 is independently 0 or 1;
each of p and p′ is independently 0-10;
each of X, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

5. An agent, wherein the agent is or comprise a peptide comprising:
X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇,
wherein:
each of p14, p15, p16 and p17 is independently 0 or 1;
each of X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or polar group;
X⁵ comprises a side chain comprising an acidic or polar group;
X⁹ comprises a side chain comprising an optionally substituted aromatic group;
X² comprises a side chain comprising an optionally substituted aromatic group;
X¹³ comprises a side chain comprising an optionally substituted aromatic group; and wherein:
X¹ and X⁴, and/or X⁴ and X¹¹ are independently amino acid residues suitable for stapling, or are stapled, or X³ and X¹⁰ are independently amino acid residues suitable for stapling, or are stapled.

6. A agent having the structure of formula I:
R^{N}-L^{P1}-L^{AA1}-L^{P2}L^{AA2}-L^{P3}-L^{AA3}-L^{P4}-L^{AA4}-L^{P5}-L^{AA5}-L^{P6}-L^{AA6}-L^{P7}-R^{C} I
or a salt thereof, wherein:
R^{N} is a peptide, an amino protecting group or R′-L^{RN}-;
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
a first R′ group and a second R′ group which are taken together to form -L^{s}- which is bonded to the atom to which a first R′ group is attached and the atom to which a second R′ group is attached; and
a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L^{s3}-, wherein each L^{s1}, L^{s2} and L^{s3} is independently L;
L^{AA1} is an amino acid residue that comprises a side chain comprising an acidic or polar group;
L^{AA2} is an amino acid residue that comprises a side chain comprising an acidic or polar group;
L^{AA3} is an amino acid residue;
L^{AA4} is an amino acid residue that comprises a side chain comprising an optionally substituted aromatic group;
L^{AA5} is an amino acid residue that comprises a side chain comprising an optionally substituted aromatic group;
L^{AA6} is an amino acid residue that comprises a side chain comprising an optionally substituted aromatic group;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁-30 aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

7. A agent having the structure of formula I:
R^{N}-L^{P1}-L^{AA1}-L^{P2}L^{AA2}-L^{P3}-L^{AA3}-L^{P4}-L^{AA4}-L^{P5}-L^{AA5}-L^{P6}-L^{AA6}-L^{P7}-R^{C} I
or a salt thereof, wherein:
R^{N} is a peptide, an amino protecting group or R′-L^{RN}-;
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
a first R′ group and a second R′ group which are taken together to form -L^{s}- which is bonded to the atom to which a first R′ group is attached and the atom to which a second R′ group is attached; and
a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L^{s3}-, wherein each L^{s1}, L^{s2} and L^{s3} is independently L;
L^{AA1} is L^{AR}, wherein a methylene unit is replaced with —C(R′)(R^{AS})—, wherein R^{AS} is -L^{AS1}-R^{AA1}, wherein R^{AA1} is CO₂R or —SO₂R;
L^{AA2} is L^{AR}, wherein a methylene unit is replaced with —C(R′)(R^{AS})—, wherein R^{AS} is -L^{AS2}-R^{AA2} wherein R^{AA2} is —CO₂R, or —SO₂R;
L^{AA3} is L^{AR}, wherein a methylene unit is replaced with —C(R′)(R^{AS})—, wherein R^{AS} is -L^{AS3}-R^{AA3} wherein R^{AA3} is R′;
L^{AA4} is L^{AR}, wherein a methylene unit is replaced with —C(R′)(R^{AS})—, wherein R^{AS} is -L^{AS4}-R^{AA4} wherein R^{AA4} is an optionally substituted group selected from 6-14 membered aryl or 5-14 membered heteroaryl having 1−6 heteroatoms;
L^{AA5} is L^{AR}, wherein a methylene unit is replaced with —C(R′)(R^{AS})—, wherein R^{AS} is -L^{AS5}-R^{AA5}, wherein R^{AA5} is an optionally substituted group selected from 6-14 membered aryl or 5-14 membered heteroaryl having 1−6 heteroatoms;
L^{AA6} is L^{AR}, wherein a methylene unit is replaced with —C(R′)(R^{AS})—, wherein R^{AS} is -L^{AS6}-R^{AA6} wherein R^{AA6} is an optionally substituted group selected from 6-14 membered aryl or 5-14 membered heteroaryl having 1−6 heteroatoms;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L^{A}R is independently an optionally substituted, bivalent C₁-C₆ aliphatic group, wherein one or more methylene units of the group are optionally and independently replaced with —C(R′)₂—, —C(R′)(R^{PS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each of L^{AS1}, L^{AS2}, L^{AS3}, L^{AS4}, L^{AS5}, and L^{AS6} is independently L^{AS};
each R^{AA}s is independently -L^{AS}-R′;
each L^{AS} is independently a covalent bond or an optionally substituted, bivalent C₁-C₁₀ aliphatic or heteroaliphatic group having 1-5 heteroatoms, wherein one or more methylene units of the group optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

8. The agent of any one of the preceding claims, wherein a second R′ group and a third R′ group are attached to the same atom, and wherein each of the first, second and fourth R′ groups is independently attached to a different atom, or:
wherein X¹ and X⁴, and X⁴ and X¹¹ are independently stapled.

9. The agent of claim 8, wherein the agent comprises a staple having the structure of L^{s} which is -L^{s1}-L^{s2}-L^{s3-}.

10. The agent of claim 9, wherein L^{s1} is a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

11. The agent of claim 10, wherein L^{s1} is bond to a backbone carbon atom or nitrogen atom, or an alpha carbon atom of an amino acid residue.

12. The agent of claim 11, wherein L^{s2} is a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

13. The agent of claim 12, wherein L^{s3} is a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

14. The agent of claim 13, wherein L^{s3} is a bivalent C₁₋₆ aliphatic wherein one or more methylene units are independently replaced with —N(R′)—.

15. The agent of claim 14, wherein L^{s3} is bond to a carbon atom of the peptide backbone, or wherein L^{s3} is bond to an alpha carbon atom of an amino acid residue, or wherein L^{s3} is bond to a nitrogen atom of the peptide backbone.

16. The agent of claim 8, wherein the agent comprises a staple having the structure of —(CH₂)m-N(R′)—C(O)—O—(CH₂)ₙ—CH═CH—(CH₂)n′-, wherein each of m, n and n′ is independently 1, 2, 3, 4, 5, 6, 7, 8, 9, or 10, and each —CH₂— is independently optionally substituted.

17. The agent of any one of the preceding claims, wherein X¹ is —N(R^{a1})-L^{a1}-C(-L^{a}-R^{SP1})(R^{a3})-L^{a2}-C(O)—.

18. The agent of any one of the preceding claims, wherein X⁴ is a residue of an amino acid that comprises two olefins each independently suitable for stapling, or wherein X⁴ has the structure of —N(R^{a1})-L^{a1}-C(-L^{a}-R^{SP1})X-L^{a}-R^{SP2})-L^{a2}-C(O)—.

19. The agent of any one of the preceding claims, wherein X⁴ is B5.

20. The agent of any one of the preceding claims, wherein X″ is an amino acid residue suitable for stapling, or wherein X″ is —N(R^{a1})-L^{a1}-C(-L^{a}-R^{SP1})(R^{a3})-L^{a2}-C(O)—.

21. The agent of any one of claims 1-5, wherein X² or L^{AA1} comprises a side chain comprising —COOH or a salt form thereof.

22. The agent of claim 21, wherein X² or L^{AA1} is Asp or Glu.

23. The agent of claim 21, wherein X⁵ or L^{AA2} comprises a side chain comprising —COOH or a salt form thereof.

24. The agent of claim 22, wherein X⁵ or L^{AA2} is Asp or Glu.

25. The agent of claim 23, wherein X⁶ or L^{AA3} comprises a side chain comprising —COOH or a salt form thereof.

26. The agent of claim 21, wherein X⁶ or L^{AA3} is Asp or Glu.

27. The agent of claim 21, wherein X⁶ or L^{AA3}

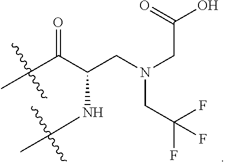 [SMILES: *N[C@@H](CN(CC(=O)O)CC(F)(F)F)C(*)=O]

28. The agent of claim 21, wherein X⁶ or L^{AA3} is

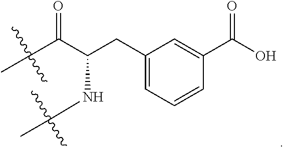 [SMILES: *N[C@@H](Cc1cccc(C(=O)O)c1)C(*)=O]

29. The agent of claim 25, wherein X⁹ or L^{AA4} comprises a side chain which is or comprises an optionally substituted aromatic group.

30. The agent of claim 29, wherein X⁹ or L^{AA4} is Phe.

31. The agent of claim 29, wherein X² or L^{A52} comprises a side chain which is or comprises an optionally substituted aromatic group.

32. The agent of claim 31, wherein X² or L^{AA5} is Phe.

33. The agent of claim 31, wherein X² or L^{AA5} is

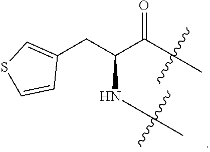 [SMILES: *N[C@@H](Cc1ccsc1)C(*)=O]

34. The agent of claim 31, wherein X² or L^{AA5} is

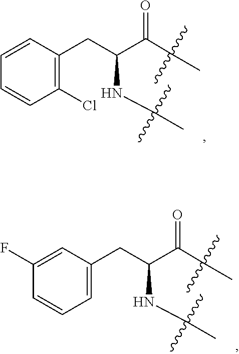 [SMILES: *N[C@@H](Cc1ccccc1Cl)C(*)=O ; *N[C@@H](Cc1cccc(F)c1)C(*)=O]

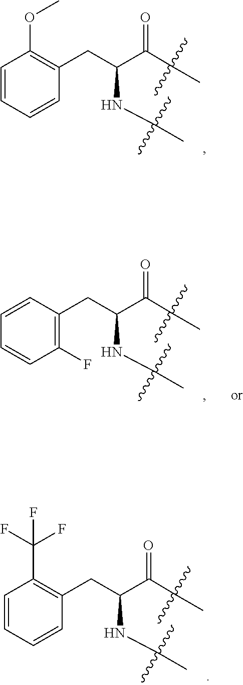 [SMILES: *N[C@@H](Cc1ccccc1OC)C(*)=O ; *N[C@@H](Cc1ccccc1F)C(*)=O ; *N[C@@H](Cc1ccccc1C(F)(F)F)C(*)=O]

35. The agent of claim 31, wherein the side chain of X¹³ or L^{AA6} comprises an optionally substituted aromatic group.

36. The agent of claim 35, wherein the side chain of X¹³ or L^{AA6} comprises an optionally substituted 9-membered bicyclic heteroaryl group having 1-3 heteroatoms.

37. The agent of claim 35, wherein X¹³ or L^{AA6} is

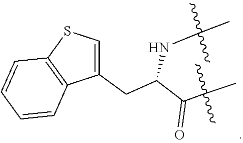 [SMILES: *N[C@@H](Cc1csc2ccccc12)C(*)=O]

38. The agent of claim 35, wherein X¹³ or L^{AA6} is Trp.

39. The agent of any one of the preceding claims, wherein p14 is 1.

40. The agent of any one of the preceding claims, wherein X¹⁴ comprises a side chain comprising a polar group.

41. The agent of any one of the preceding claims, wherein the peptide has the structure of:
R^{N}—[X]ₚ-X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇—[X]_{p′}-R^{C},
or a salt thereof, wherein:
each X is independently an amino acid residue;
each p and p′ is independently 0-10;
R^{N} is independently a peptide, an amino protecting group or R′-L^{RN}-;
R^{C} is independently a peptide, a carboxyl protecting group, -L^{RC}-R′, —O-L^{RC}-R′ or —N(R′)-L^{RC}-R′;
each of L^{RN} and L^{RC} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

42. The agent of any one of the preceding claims, wherein R^{N} is —C(O)R.

43. The agent of any one of the preceding claims, wherein R^{N} is Ac, AzAc (N₃—CH₂—C(O)—), 2PyPrpc 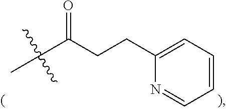 [SMILES: *C(=O)CCc1ccccn1]
MeOPr (CH₃OCH₂CH₂C(O)—), MeSO₂ (—SO₂CH₃), mPEG2 (CH₃OCH₂CH₂OCH₂CH₂C(O)—), Nic 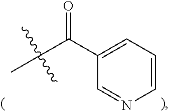 [SMILES: *C(=O)c1cccnc1]
Oct (CH₃(CH₂)₆C(O)—), or Pic

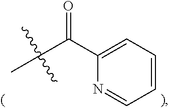 [SMILES: *C(=O)c1ccccn1]

44. The agent of any one of the preceding claims, wherein R^{C} is —N(R′)₂, or wherein R^{C} is —NH₂, —NHEt, —NHBn, 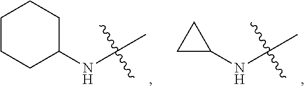 [SMILES: *NC1CCCCC1 ; *NC1CC1]
—NH—(CH₂)₆—NH₂, —NH—(CH₂)₆—N₃, 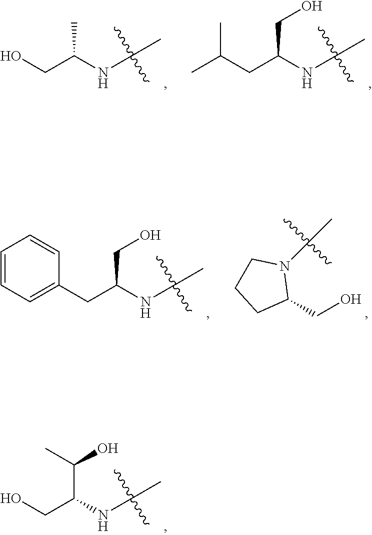
or —OH.

45. The agent of any one of the preceding claims, wherein the peptide forms a structure that comprises a helix.

46. The agent of any one of the preceding claims, wherein the peptide binds to beta-catenin with a EC50 of no more than about 2000 nM, or no more than about 1500 nM, or no more than about 1000 nM, or no more than about 500 nM, or no more than about 300 nM, or no more than about 200 nM, or no more than about 100 nM, or no more than about 75 nM, or no more than about 50 nM, or no more than about 25 nM, or no more than about 10 nM as measured by fluorescence polarization.

47. The agent of any one of the preceding claims, wherein the peptide binds to a polypeptide whose sequence is or comprising SEQ ID NO: 2, or a fragment thereof:
(SEQ ID NO: 2)
SVLFYAITTLHNLLLHQEGAKMAVRLAGGLQKMVALLNKTNVKFLAITT
DCLQILAYGNQESKLIILASGGPQALVNIMRTYTYEKLLWTTSRVLKVL
SVCSSNKPAIVEAGGMQALGLHLTDPSQRLVQNCLWTLRNLSDAATKQE
GMEGLLGTLVQLLGSDDINVVTCAAGILSNLTCNNYKNKMMVCQVGGIE
ALVRT.

48. The agent of any one of the preceding claims, wherein the agent interacts with G307 of beta-catenin or an amino acid residue corresponding thereto, K312 of beta-catenin or an amino acid residue corresponding thereto, K345 of beta-catenin or an amino acid residue corresponding thereto, W383 of beta-catenin or an amino acid residue corresponding thereto, N387 of beta-catenin or an amino acid residue corresponding thereto, D413 of beta-catenin or an amino acid residue corresponding thereto, and/or N415 of beta-catenin or an amino acid residue corresponding thereto.

49. An agent having a structure selected from Table E3 or a salt thereof.

50. An agent has the structure of 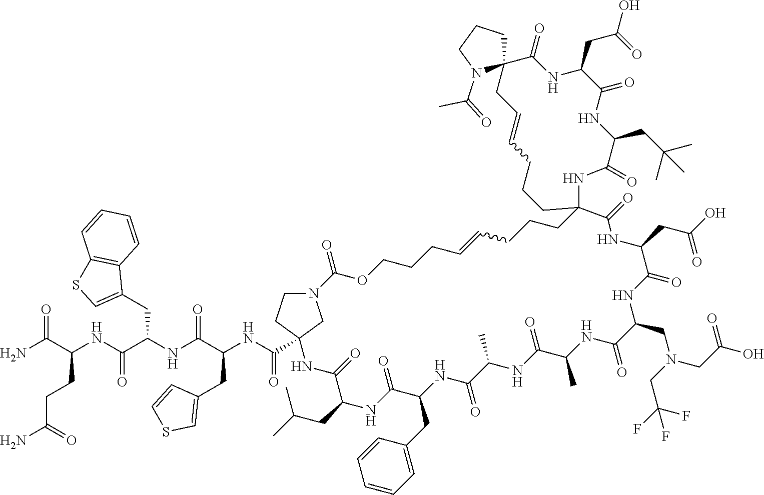 [SMILES: CC(=O)N1CCC[C@]12CC=CCCCC1(CCCC=CCCCOC(=O)N3CC[C@@](C(=O)N[C@@H](Cc4ccsc4)C(=O)N[C@@H](Cc4csc5ccccc45)C(=O)N[C@@H](CCC(N)=O)C(N)=O)(C3)NC(=O)[C@H](CC(C)C)NC(=O)[C@H](Cc3ccccc3)NC(=O)[C@H](C)NC(=O)[C@H](C)NC(=O)[C@H](CN(CC(=O)O)CC(F)(F)F)NC(=O)[C@H](CC(=O)O)NC1=O)NC(=O)[C@H](CC(C)(C)C)NC(=O)[C@H](CC(=O)O)NC2=O]
or a salt thereof; or
an agent has the structure of 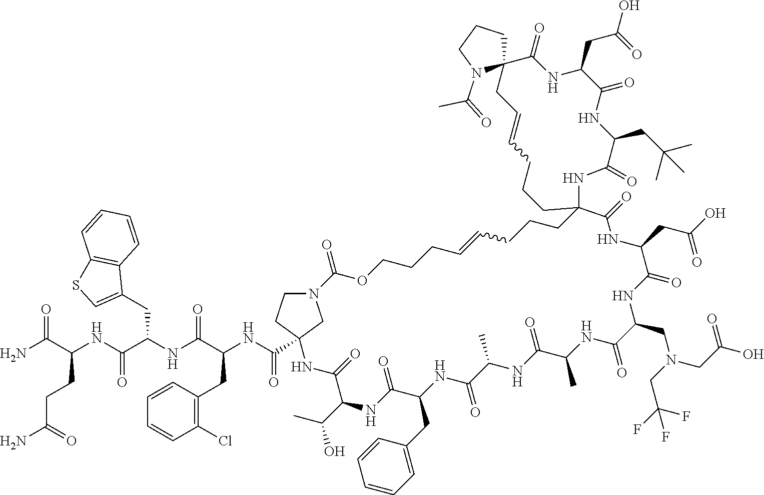 [SMILES: CC(=O)N1CCC[C@]12CC=CCCCC1(CCCC=CCCCOC(=O)N3CC[C@@](C(=O)N[C@@H](Cc4ccccc4Cl)C(=O)N[C@@H](Cc4csc5ccccc45)C(=O)N[C@@H](CCC(N)=O)C(N)=O)(C3)NC(=O)[C@H]([C@@H](C)O)NC(=O)[C@H](Cc3ccccc3)NC(=O)[C@H](C)NC(=O)[C@H](C)NC(=O)[C@H](CN(CC(=O)O)CC(F)(F)F)NC(=O)[C@H](CC(=O)O)NC1=O)NC(=O)[C@H](CC(C)(C)C)NC(=O)[C@H](CC(=O)O)NC2=O]
or a salt thereof; or
an agent has the structure of 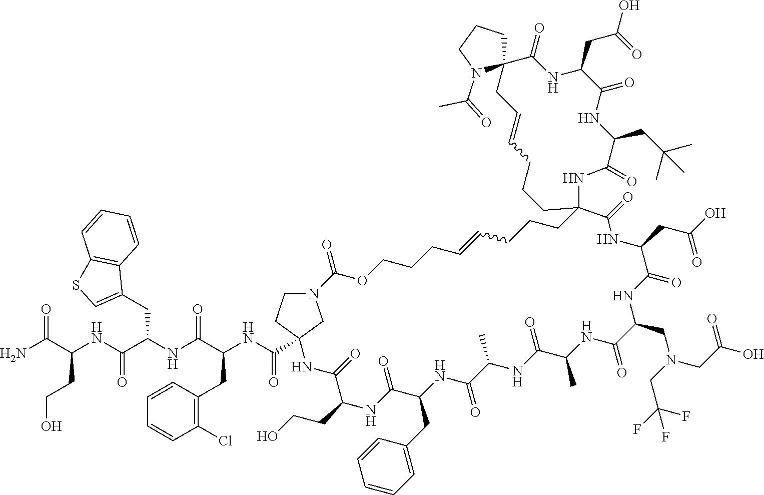 [SMILES: CC(=O)N1CCC[C@]12CC=CCCCC1(CCCC=CCCCOC(=O)N3CC[C@@](C(=O)N[C@@H](Cc4ccccc4Cl)C(=O)N[C@@H](Cc4csc5ccccc45)C(=O)N[C@@H](CCO)C(N)=O)(C3)NC(=O)[C@H](CCO)NC(=O)[C@H](Cc3ccccc3)NC(=O)[C@H](C)NC(=O)[C@H](C)NC(=O)[C@H](CN(CC(=O)O)CC(F)(F)F)NC(=O)[C@H](CC(=O)O)NC1=O)NC(=O)[C@H](CC(C)(C)C)NC(=O)[C@H](CC(=O)O)NC2=O]
or a salt thereof.

51. The agent of claim 50, wherein a double bond of a staple bonded to the first amino acid from the N-terminus is E.

52. The agent of claim 50, wherein a double bond of a staple bonded to the first amino acid from the N-terminus is Z.

53. The agent of any one of claims 50-52, wherein a double bond of a staple bonded to the 11th amino acid from the N-terminus is E.

54. The agent of any one of claims 50-52, wherein a double bond of a staple bonded to the 11^{th} amino acid from the N-terminus is Z.

55. A compound having the structure of formula PA:
N(R^{PA})(R^{a1})-L^{a1}-C(R^{a2})(R^{a3})-L^{a2}-C(O)R^{PC} PA
or a salt thereof, wherein:
R^{PA} is —H or an amino protecting group;
each of R^{a1} and R^{a3} is independently -L^{a}-R′;
R^{a2} is -L^{aa}-C(O)R^{PS};
each of L^{a}, L^{a1} and L^{a2} is independently L;
—C(O)R^{PS} is optionally protected or activated —COOH;
—C(O)R^{C} is optionally protected or activated —COOH;
each L is independently a covalent bond, or an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently —R, —C(O)R, —CO₂R, or —SO₂R; and
each R is independently —H, or an optionally substituted group selected from C₁-30 aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

56. The compound of claim 55, wherein L^{aa} is L and L^{aa} comprises —N(R′)— or -Cy-.

57. The compound of claim 55, having the structure of: 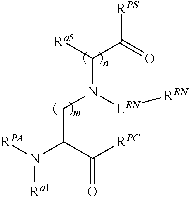
or a salt thereof, wherein:
each of m and n is independently 1, 2, 3, or 4;
L^{RN} is L;
R^{RN} is R; and
R^{a5} is R′.

58. The compound of claim 55, wherein the compound has the structure of 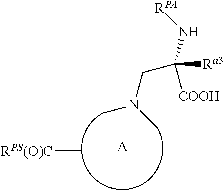
or a salt thereof, wherein Ring A is an optionally substituted 3-7 membered saturated, partially unsaturated or aromatic ring; or
wherein the compound has the structure of 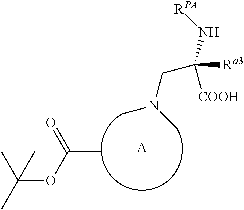
or a salt thereof, wherein Ring A is an optionally substituted 3-7 membered saturated, partially unsaturated or aromatic ring; or
wherein the compound has the structure of 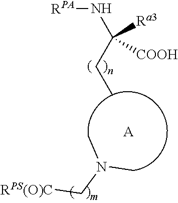
or a salt thereof, wherein:
Ring A is an optionally substituted 3-10 membered ring;
n is 0, 1, or 2; and
m is 0, 1, 2, or 3; or
wherein the compound has the structure of 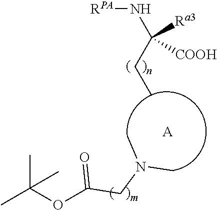
or a salt thereof, wherein:
Ring A is an optionally substituted 3-10 membered ring;
n is 0, 1, or 2; and
m is 0, 1, 2, or 3; or
wherein the compound has the structure of 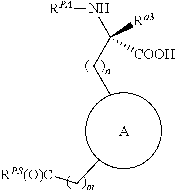
or a salt thereof, wherein:
Ring A is an optionally substituted 3-10 membered ring;
n is 0, 1, or 2; and
m is 0, 1, 2, or 3; or
wherein the compound has the structure of 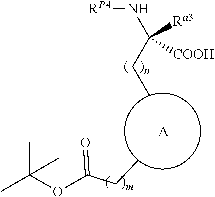
or a salt thereof, wherein:
Ring A is an optionally substituted 3-10 membered ring;
n is 0, 1, or 2; and
m is 0, 1, 2, or 3; or
wherein the compound has the structure of 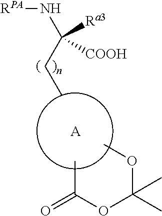
or a salt thereof, wherein:
Ring A is an optionally substituted 3-10 membered ring; and
n is 0, 1, or 2.

59. A compound having the structure of: 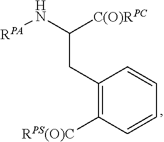
or a salt thereof, wherein:
R^{PA} is —H or an amino protecting group;
—C(O)R^{PS} is optionally protected or activated —COOH; and
—C(O)R^{C} is optionally protected or activated —COOH.

60. A compound having the structure of: 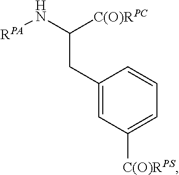
or a salt thereof, wherein:
R^{PA} is —H or an amino protecting group;
—C(O)R^{PS} is optionally protected or activated —COOH; and
—C(O)R^{C} is optionally protected or activated —COOH.

61. The compound of any one of claims 55-61, wherein RA is an amino protecting group suitable for peptide synthesis.

62. The compound of any one of claims 55-61, wherein R^{PA} is —C(O)—O—R; or wherein R^{PA} is -Fmoc.

63. The compound of any one of claims 55-62, wherein —C(O)R^{PS} is —C(O)OR′.

64. The compound of claim 63, wherein R′ is —H.

65. The compound of claim 63, wherein R′ is optionally substituted C₁₋₆ aliphatic.

66. The compound of any one of claims 55-62, —C(O)R^{PS} is —C(O)S-L-R′.

67. A compound, wherein the compound is 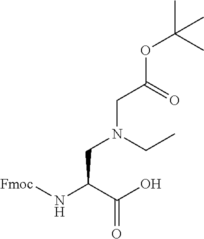
or a salt thereof, or wherein the compound is 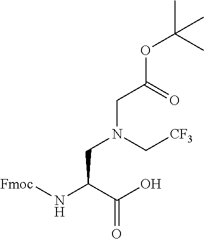
or a salt thereof.

68. A compound, wherein the compound is 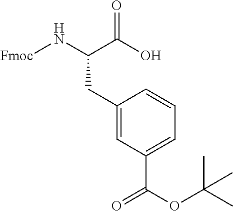
or a salt thereof.

69. A compound, wherein the compound is 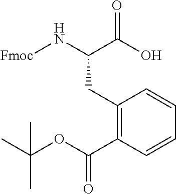
or a salt thereof.

70. A compound, wherein the compound is 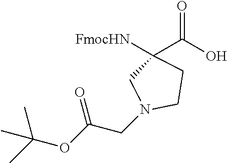
or a salt thereof.

71. A compound, wherein the compound is 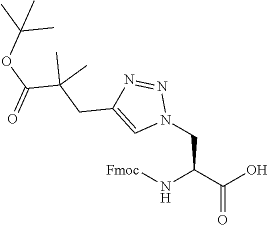
or a salt thereof.

72. A compound, wherein the compound is 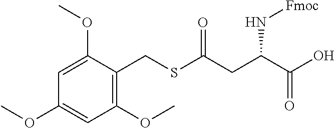
or a salt thereof, or wherein the compound is 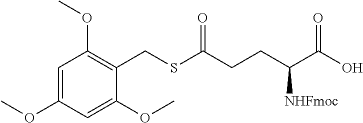
or a salt thereof.

73. A compound, wherein the compound is 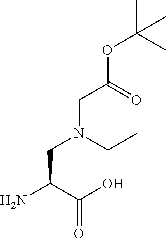 [SMILES: CCN(CC(=O)OC(C)(C)C)C[C@H](N)C(=O)O]
or a salt thereof, or wherein the compound is 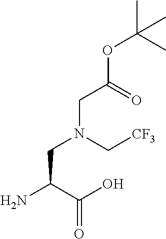 [SMILES: CC(C)(C)OC(=O)CN(C[C@H](N)C(=O)O)CC(F)(F)F]
or a salt thereof, or wherein the compound is 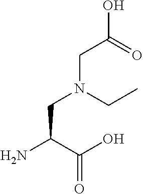
or a salt thereof, or wherein the compound is 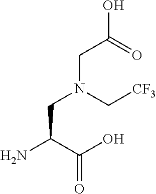 [SMILES: N[C@@H](CN(CC(=O)O)CC(F)(F)F)C(=O)O]
or a salt thereof.

74. A compound, wherein the compound is 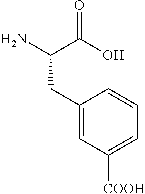 [SMILES: N[C@@H](Cc1cccc(C(=O)O)c1)C(=O)O]
or a salt thereof.

75. A compound, wherein the compound is 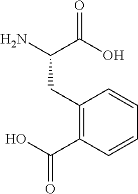 [SMILES: N[C@@H](Cc1ccccc1C(=O)O)C(=O)O]
or a salt thereof.

76. A compound, wherein the compound is 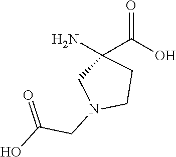 [SMILES: N[C@@]1(C(=O)O)CCN(CC(=O)O)C1]
or a salt thereof.

77. A compound, wherein the compound is 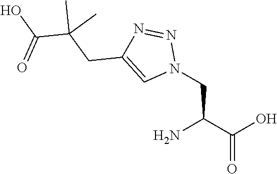 [SMILES: CC(C)(Cc1cn(C[C@H](N)C(=O)O)nn1)C(=O)O]
or a salt thereof.

78. A compound, wherein the compound is 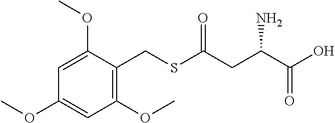 [SMILES: COc1cc(OC)c(CSC(=O)C[C@H](N)C(=O)O)c(OC)c1]
or a salt thereof, or wherein the compound is 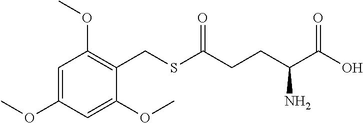 [SMILES: COc1cc(OC)c(CSC(=O)CC[C@H](N)C(=O)O)c(OC)c1]
or a salt thereof, wherein the compound is 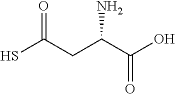
or a salt thereof, or wherein the compound is 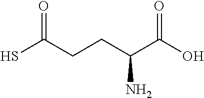
or a salt thereof.

79. The compound of any one of the preceding claims, wherein the compound has a purity of at least about 90%, 91%, 92%, 93%, 94%, 95%, 96%, 97%, 98%, or 99%.

80. A compound, comprising a residue of Table A-IV.

81. A compound, comprising a residue having the structure of 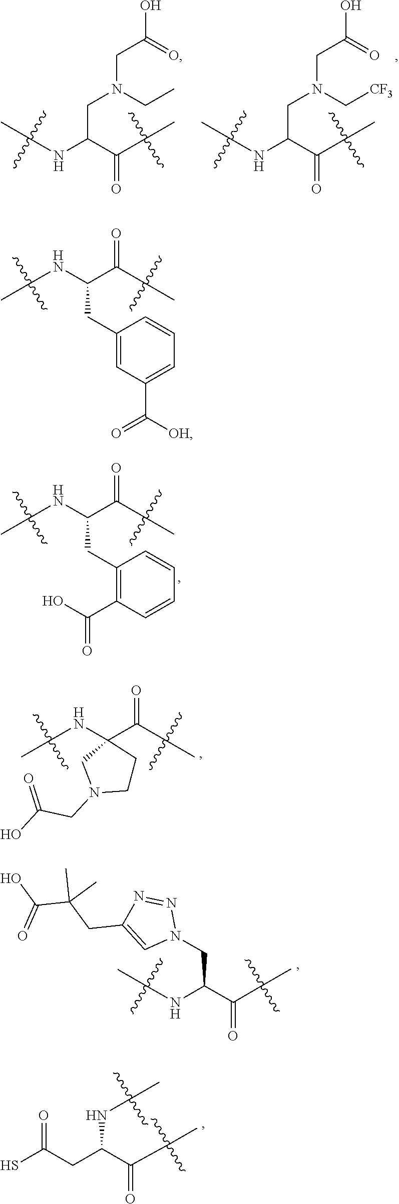 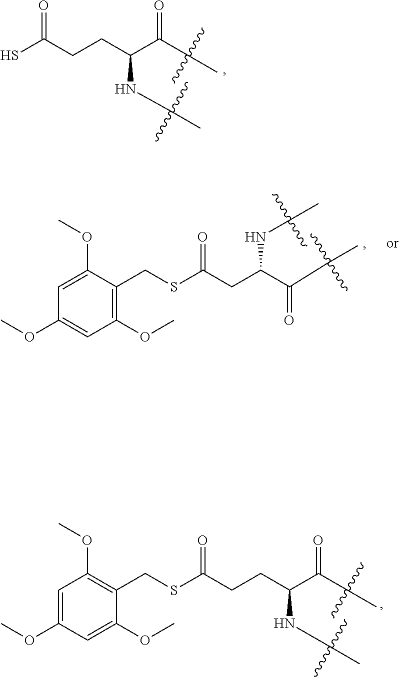 [SMILES: *N[C@@H](CCC(=O)S)C(*)=O ; *N[C@@H](CC(=O)SCc1c(OC)cc(OC)cc1OC)C(*)=O ; *N[C@@H](CCC(=O)SCc1c(OC)cc(OC)cc1OC)C(*)=O]
salt form thereof.

82. The compound of claim 81, wherein the compound is or comprise a peptide, or wherein the compound is or comprise a stapled peptide.

83. A method for preparing a compound of claim 81 or 82, comprising utilization of a compound of any one of the claims 55-79.

84. The agent of any one of the preceding claims, wherein each olefin double bond in a staple is independently and optionally converted into a single bond, or wherein each olefin double bond in a staple is converted into a single bond, or wherein each olefin double bond is converted into a single bond, or wherein each olefin double bond is independently and optionally converted into —CHR′—CHR′—, wherein each R is independently —H, —R, —OR, —OH, —N(R)₂, or —SR, or wherein each olefin double bond is converted into —CHR″—CHR″—, wherein each R is independently —H, —R, —OR, —OH, —N(R)₂, or —SR, or wherein each olefin double bond is independently and optionally converted into optionally substituted —CH₂—CH₂—, or wherein each olefin double bond is converted into —CH₂—CH₂—.

85. A pharmaceutical composition, comprising or delivering an agent or amino acid of any one of the preceding claims, and a pharmaceutically acceptable carrier; or
a composition selected from Table E3; or
a pharmaceutical composition, comprising or delivering one or more or all peptide agents in a composition selected from Table E3 and a pharmaceutically acceptable carrier.

86. A method for preparing an agent of any one of the preceding claims, comprising incorporating a residue of an amino acid of any one of the preceding claims.

87. A method for modulating beta-catenin interaction with a partner in a system, comprising contacting beta-catenin with an agent or composition of any one of the preceding claims; or
a method for modulating beta-catenin interaction with a partner in a system, comprising administering or delivering to the system an agent or composition of any one of the preceding claims; or
a method for modulating a TCF-beta-catenin interaction in a system, comprising contacting beta-catenin with an agent or composition of any one of the preceding claims; or
a method for modulating a TCF-beta-catenin interaction in a system, comprising administering or delivering to the system an agent or composition of any one of the preceding claims; or
a method for inhibiting beta-catenin dependent cell proliferation, comprising administering or delivering to the system an agent or composition of any one of the preceding claims; or
a method for modulating a TCF-beta-catenin interaction in a system, comprising administering or delivering to the system an agent or composition of any one of the preceding claims.

88. The method of claim 87, wherein a system is an in vitro system, or wherein a system is or comprises a cell, tissue or organ, or wherein a system is a subject.

89. A method for treating or preventing a condition, disorder or disease associated with beta-catenin in a subject, comprising administering or delivering to the subject an effective amount of an agent or composition of any one of the preceding claims; or
a method for treating cancer in a subject, comprising administering or delivering to the subject an effective amount of an agent or composition of any one of the preceding claims; or
a method for treating or preventing a condition, disorder or disease associated with beta-catenin interaction with a partner in a subject, comprising administering or delivering to the subject an effective amount of an agent or composition of any one of the preceding claims.

90. The method of claim 89, wherein the partner is TCF7, LEF1, TCF7L1, TCF7L2, Axin1, Axin2, or APC.

91. A method for treating or preventing a condition, disorder or disease associated with TCF-beta-catenin interaction in a subject, comprising administering or delivering to the subject an effective amount of an agent or composition of any one of the preceding claims.

92. The method of any one of the preceding claims, wherein the condition, disorder or disease is melanoma.

93. The method of any one of the preceding claims, comprising administering or deliver to a subject a second therapeutic agent or a second therapy.

94. The method of claim 93, wherein a second therapeutic agent or therapy is administered prior to an agent of any one of the preceding claims, or wherein a second therapeutic agent or therapy is administered about or no more than about 1, 2, 3, 4, 5, 6, or 7 days, or 1, 2, 3, or weeks, or 1, 2, 3, 4, 5, or 6 months, prior to an agent of any one of the preceding claims, or wherein a second therapeutic agent or therapy is administered concurrently with an agent of any one of the preceding claims, or wherein a second therapeutic agent or therapy is administered subsequently to an agent of any one of the preceding claims, or wherein a second therapeutic agent or therapy is administered about or no more than about 1, 2, 3, 4, 5, 6, or 7 days, or 1, 2, 3, or weeks, or 1, 2, 3, 4, 5, or 6 months, subsequently to an agent of any one of the preceding claims; and/or:
wherein a subject is exposed to a second therapeutic agent or therapy and an agent of any one of the preceding claims, or wherein a subject is exposed to a therapeutic effect of a second therapeutic agent or therapy and a therapeutic effect of an agent of any one of the preceding claims; and/or:
wherein a second therapeutic agent is or comprises a chemotherapy agent, or wherein a second therapeutic agent is or comprises a hormone therapy agent, or wherein a second therapeutic agent is or comprises an immunotherapy agent, or wherein a second therapeutic agent is or comprises a checkpoint inhibitor, or wherein a second therapeutic agent is or comprises an antibody, or wherein a second therapeutic agent is or comprises a CTLA-4, PD-1 or PD-L1 inhibitor, or wherein a second therapeutic agent is or comprises a cell; and/or:
wherein the second therapeutic agent reduces one or more side effects of an agent or composition of any one of the preceding claims, or wherein the agent or composition reduces one or more side effects of a second therapeutic agent; and/or:
wherein a second therapy is or comprises surgery, or wherein a second therapy is or comprises chemotherapy, or wherein a second therapy is or comprises radiotherapy, or wherein a second therapy is or comprises hormone therapy, or wherein a second therapy is or comprises stem cell or bone marrow transplant, or wherein a second therapy is or comprises immunotherapy, or wherein a second therapy is or comprises T-cell therapy, or wherein a second therapy is or comprises CAR T-cell therapy, or wherein a second therapy is or comprises administering to the subject a population of immune cells, or wherein the agent or composition reduces one or more side effects of a second therapy; and/or:
wherein unit dose of a second therapy or therapeutic agent is reduced compared to when it is administered alone, or wherein total dose of a second therapy or therapeutic agent is reduced compared to when it is administered alone, or wherein unit dose of an agent or composition of any one of the preceding claims is reduced compared to when it is administered alone, or wherein total dose of an agent or composition of any one of the preceding claims is reduced compared to when it is administered alone; and/or:
wherein the combination therapy provides higher efficacy than when an agent or composition is administered or delivered alone, or wherein the combination therapy provides higher efficacy than when a second therapeutic agent or therapy is administered or delivered alone.

95. An agent, compound, composition or method described in the specification or Embodiments 1-1241.
